SEQUENCE LISTING (466 sequences)

SEQ ID NO: 1 - protein, 408 aa, Deinococcus radiodurans
MIRNKAFVVRLYPNAAQTELINRTLGSARFVYNHFLARRIAAYKESGKGLTYGQTSSELTLLKQAEETSWLSEVDKFALQNSLKNLETAYKNFFRTVKQSGKKVGFPRFRKKRTGESYRTQFTNNNIQIGEGRLKLPKLGWVKTKGQQDIQGKILNVTVRRIHEGHYEASVLCEVEIPYLPAAPKFAAGVDVGIKDFAIVTDGVRFKHEQNPKYYRSTLKRLRKAQQTLSRRKKGSARYGKAKTKLARIHKRIVNKRQDFLHKLTTSLVREYEIIGTEHLKPDNMRKNRRLALSISDAGWGEFIRQLEYKAAWYGRLVSKVSPYFPSSQLCHDCGFKNPEVKNLAVRTWTCPNCGETHDRDENAALNIRREALVAAGISDTLNAHGGYVRPASAGNGLRSENHATLVV

SEQ ID NO: 2 - protein, 630 aa, Staphylococcus aureus
MNASSKRKIISQSEISKKIAVMNEEMQGFWANNSWDIRKCPHPSAIELSKNPALRNRWVRFERVKNLWLRTELKYFYFYHLNNGIWNAKTVWIRKGTVINKMLDFLDLKYPSITSITEVPIDKAMTEYRTYLTKQGVRITTTNYKITANQEKTPVKANSYYVTNLKQFIEFIEDFYFDGEEWDKDVWDRRNLPLPDDKVNPTQYEYTINFKGFRNTYFKQLVKRYCKLRLNMDSFSYVSDIAQKLKEFFNFLDIKFKHVQRVHQLTRVEIEAYLSELNMMGIKPSTITGRISILEGLFSTLHRLEWDDVPSKLLIYPEDYPKIPRAKPRFIDEFVLEQLNSHLDKLPEYIATMTMIVQECGMRISELCTLKKGCLLEDKDGYYFLKYYQWKMKKEHIVPISKEVVLLIKVREDKVSEEFPDSEYLFPRKDGSPLKQETFRGELNKLAYEQNIVDKLGEIYRFHAHAFRHSVGTRTINNGVPQHIVQKFLGHESPEMTSRYAHIFDETLKNEFTKFQEKLVTNNGDVLNLDDDSEVDDVELQWFKKNINAQVLPNGYCRLPVIAGGCPHANACLDCTHFCTSKQFLPQHEEQLERTEELLTIAKDKQWQRQIETNSRVKERLEQIIGSLTG

SEQ ID NO: 3 - protein, 87 aa, Staphylococcus aureus
TYFKQLVKRYCKLRLNMDSFSYVSDIAQKLKEFFNFLDIKFKHVQRVHQLTRVEIEAYLSELNMMGIKPSTITGRISILEGLFSTLH

SEQ ID NO: 4 - protein, 188 aa, Staphylococcus aureus
AKPRFIDEFVLEQLNSHLDKLPEYIATMTMIVQECGMRISELCTLKKGCLLEDKDGYYFLKYYQWKMKKEHIVPISKEVVLLIKVREDKVSEEFPDSEYLFPRKDGSPLKQETFRGELNKLAYEQNIVDKLGEIYRFHAHAFRHSVGTRTINNGVPQHIVQKFLGHESPEMTSRYAHIFDETLKNEFT

SEQ ID NO: 5 - protein, 815 aa, synthetic construct
MPSTSSCKEPKSTSKPSTTSNAKKQNTKTNDKPQNDKIKKARVVKDNTFHHYTLVRGLNNMIRPEKSEQLMFAINELVPLVSKSMVLGSWHTHVRLMEIELEANRIEQETGIRVENQLGIRAEFTPPSVINARGQVRKIGYVVEKDFNPTKQTRGINLIHTEPINANFKRHLDVYDVDIPHLPYGANCHNYATNQYTTNFHNNIRYRAYNRVRAFFNVHSNDRKSKRETLKYLFYVEPKKRKAIPDPLPIPNDELLKLYRKVLRPLLPNDAPDRVPLGWLGASLRDKIDWYRHVHEFIQLQLWIYSDNLRRDGTDEIQLRSFTAIPQNKVKRHHITITTRILGGIVNSFECRAEKISSKDYESSDVAIRTRIWNKYFRLEWLNSEWRYFDLTMTTDGVSAHFVMRNKQKQKSKREVVDVNGDVLTPEIVGFIDDRIVREISDEMRQKYNNNEYKTIIGYDPGYKLPVAIVRKDIATGDETNFTMNWYEFRKLCGDVERKQKRYRVAGKHEKKMRQLRENRTIYPEQPSNKSHDIWTYIDYQCRVFHRSCAVYMRRSYARLEFDRYICTQRALHKIARIIMPRVDFKNPTRDCILAAGDFKFSANSPIRGSVRCPMRSLFNLLAQQYCDVVEIDEHLTSKLCSICGSRMIKKGTRKDRYFICKNKMPATSILPQAPAWTTKRHIDRIRDEFNELPLAGLFIHQDNGGRRIKRTIDLPTERRFHSNELDDHHYQLQQHEIINGNIVAKHKTIVWNRDINAPRNMIYKLLCMLNGVDISPVFKRRPRLAEQQQQQQQQANDNIDDTDSGSSSSDEDMR

SEQ ID NO: 6 - protein, 698 aa, synthetic construct
MAQKAVVCAYKKKFRQVNEFNINPNQIIAEHRRHAEQLTRLKRKASEHIFFRLTSLVREGTGHQILLEFQTPNRVNFRRDINSVRITERNARSEPNAEFVEGSRVYQIHPEGNIVAQSIEPIITEFEVNFANSFIGECYGRVKRLLKCFARIHLENMEDDQIKIEIRNTMRYLFNDINDITEPNRTLIRLYRQELRPICRGQVPRGWWNNKSTWYSRVPDLINGLYYLKAYNDGQRIGRDCFKLFTVIPRFKSHFVHKTKIDTRLLCLTMNKFLPEGQKLDSQNIDYTVWNRVFDMTPLERTHTDRKLVFNYSITTDAVSMSATMIQGTDREIEAKRELRGRGAKLAERKRLADERKMEESTKATKRKAGKDADEPPAKRRNAPVPTRERDLLQKLKSGEVFGTVIQIDPGVKNTLGCVAIDMQNGHERNLLITSKYYHHLLQTHLRKERKRPIIAHLHAMIRGVHASYANPPTRKGGDIREYLDSVFRLFDMFTEVYQRRVFSRMRFDAHICKQKAVDKISKQIASLNPHQESATLIMFGNSQMSRKSPIKGYVKSPHKNYWVKQFDELERVEVYEVDEYRTTKTCSFCGQILHVTRNIGHRYVSCKDCYPAGNASMVNEPKEVQTKSKQYWRRHHYRVNGIRTFSYSWEERMSHRKCRTTNRDINAPRNIGLKGFATITGRENLLPVALIRANDND

SEQ ID NO: 7 - protein, 620 aa, synthetic construct
MLRTMYQHSLLWPLITFFLNFANWFDRTIIMIWRKNFIAKQTANMFWDYFYEIKFWIHDKSSNLDRDYAKMLTKSVKNKPNLKNLNQPIKFVINQFKMNFENSTKMHAEKHVKKFIKCVNKQFFRDKNDEQRTEIIENAISFLFDKKSDCKINKLVLREINNHVRVCLGQITRGYFATMKSSGWYKFTPENIKMQWYINRYNLAYPDDQLPSFKAVPTHNLGLKCVRIDTVSLKDLFPIPVKDYKNPKSELWSRYFNVSPIIGDRKNLYFDNSIITNGPYTNFQFIISTTPINTDLPKKIIGQNASCATGKDFKIATINDSIRQSVRNNEFDVITAIDIGFNNMAGTTSLFSFDISELSLNYTSKWFRSIAGFMWHKLERERITQVFQEYEKDLRESHQYQPSVNAVDFMLYIRHAYHRNDLARLRFSQQQMKQKAVATLGYSIIGTRPDGTKNNALIGIGAMHFSGSSPIKGHGRVPHKALCAYLKDRSTFIRVSEFRSTQACCFCGKMSVESSGKDRYVVCKDCAPNQNFLLPPAKLFTVKTHNNFRRLYGFKLPFRSYERTNSNSMINTRNTNRDLCGSRNILQIMLYSVHKQPLPGHLNNSNDPKKVKKVSPKIKK

SEQ ID NO: 8 - protein, 731 aa, synthetic construct
MHFSTPYINRFWRFKNHSAAGHHKKQKYIQVNALTIVHCIHFINEIFSKRTVVFSVFFKWFCCKMRQFTIKTTLRSICYDEDLIAAIDRLSRDVSIIQFLASHHLHKRSMEIIQSENPFRIRREFDREVTDLMFKNDFNAVKANTRNPVADAQFLRWMANCNPPVQLPSGLHSGNVLQEAMKMYKTNYSNNIKMRAKSMFRKYCKAVLYNTGRVWPQRVLNKLINLAVDWFFFFTNYQPRRGSPVLTIIEEFTRNIRINNEAVDRGFLSRSLDGRDFYEFSKYFIKMQLFVYEYNDMHPDRKVKSFSVIPLHNYHRRCVLIDTETLRKLTHMHMDKKQPNHLGNLNPKKSDVWTHFDMSQVDYKRDNQRFDFKITSDGFQANILMMKLRNDELHVDLDTPSKMKGQSMRPKDLPMLENFDQIPAEVRARVDEYHVKIGIDPGAKTMASWTKQYRSLAEDCDSIESKTFREMFGFREREYNRRKITRWFDSRERRAREESDIPITRKGPHYTEFVQHKLEFCNAGFAVYHDPELRKMKFDAHQLRLKAMYAVAKRIIGYSGNKKLNEALIFFGHWRWTANSPIRGIRTPLKLLRFCLQQIGSTVIVIHEHRTSKLCNFCGQTMIHDDRNSLCIHCEPIERTLAPATVWQMRAHKSFLKRHPFAPRMRLYTRDPNQPMRRNNQGHNPMNWNRDFNASRAILSKGLDLVAGRPLPRNYLQTTQRVVQRVRFPRR

SEQ ID NO: 9 - protein, 656 aa, synthetic construct
MRRFTIKTTLRSICESDRLIADIKTMAEDISKIQTLASLHIHKRSMDIILSNDPVRIRAEFDQPVEGKMFRKDFHAVKANTRNRVADAEFMEWVRDAGIQLPRARNTDQALISSYEMYKTNYSNHIKMRAKQMFRKFCKSVLRRFYQQRERWYNEQLNRAVEYFFYFTNYRPTRFIQKIIQEFNQFIRIENENVGRGFMNHSLDGDYFYEFAKYFLKMKLYIHRYNDAHPNRKVQTIKVIPIHNFHRRCITIDTSTLRKLTHTYGNSPLAALNPRKSDVWSDFDMSSLDIKDQYFDYQITTDGFAANICMIKSRVYVDTELPSKIKGVNMRVFEPPAQEPPFDQIPDEVIERIDQFDLQIGIDPGGKTMAAVTQKFPNDDENCFSIEAKKYREMTGFRTREYNRRGITGYFDEWEKQIRQQSYIPITSKGRHYREFVQHKLDMCDWGWEAYHHPKVIQMKFDAHQQKLKAMYAVAKSIIGYSAENQRWNEALIFFGNWTWKANSPISGTRTPIRLLRFCLQQLGSTVIVIDEYRTSMLCNFCSLRMIEEGRNELCVDCVPIDRSLAPAAVWQIKAHKSYLKRNPGAYKIRTFTRNPNQPLRRNHQNHSPMNWNRDYNASRAILSKGLDLVAGRQVPHNYLRTTPNVPRRQRFRRVR

SEQ ID NO: 10 - protein, 661 aa, synthetic construct
MRQFVIKRKLRSILNDADLINRIDQIAENVSNIQFLASHYLHKRSMQVILSEDAALIRREFDRPVRDLMFKSDFFAVKANTRNAERDADFMQWLADVNPPILLPDALHCDNALQHAMKMYKTNYANNIKMRAKQMFRKFCKAVLYSTGRIWPRIVLNKLINRAVDWFFYFTRYEPNDRSPVRTIIDEFIWDILIDDEDVGRGFLSGSLDARNFYQFAKYFIKMQLYVHHYNDTHPERMVKSFKVIPLHNYHKRMVMIDTETMKKMRHMHFDKKIHNINPPKSDVWAPFRLEGLEKINQRFDFKIMSDGVTAAILMMKLNPHEFHIDLNLPSKINGVSTRPKDLPLKERFDHIPRGAIENINFDAKIGIDPGAKTMAACTKQHQNRDLTEECFSIESKKYRELAGTRKNEYNRRSITGRFDKREREAREASEILITSNGPDYAEFVRHKLEFFNEGRRVYHHPYVRELKFDAHQEKLAAMYAIAKEIIGRSANRWNKALISFGHWRWTANSPIRGIRTPLKLLRFCLQQLGSTVIVIDEHRTSKLCNFCAQTMINEDRNSLCVDCRPIQRTLAPAAVWRIKAHKSYLKRNPGAPKIREYTRDPNQQLERNNQNHSPMNWNRDYNGSRAILSKGLDLIAGHPLPRNYLRTTQRVVQRRRFARR

SEQ ID NO: 11 - protein, 772 aa, synthetic construct
MAVGSKENVSVHLSNDPKEEITVVKRTLNSILSDVNPNKERIQRAIDDRVDRVTRMSYLASTLFLYECYKAVENRNFAYFDTVDNIKACHYKVTNSTDRKYVGRLQQCRDIWNLFQTFGIDQIDTSYFGNIMNYSYGEYTTNCQNNITLHFKQRIKRFFKVIANTPNNNLPNFTQETIEDTVKYMNDTRATVQPNLELIHQIPYWEELHINDDHINAHPGFFRDFINHGENWFKAIPVFIQIQWAINDYQILCQTNEELPNFKNFVVIPQNTHKLRHIRIDNQAFFNILAELDIVPPSKKNKKRKNDGSLPRMDVVEFIKRKEENWYSLLDEDKIRQSERQKGEKPEIRKPKKGRPARRKGRGKPIVDRTKAKAFNQQIVTNGVSASVFYINNQRDRAVKPDDYLEEAIPQKLQNDQYRRVIGVDPGYKVWTSAVIRYPHKKKEFNYEVPSKKFHGQCGKWKREKKQKELTGEFEASRNRDALDRDLYAETPSANSRQHWFNYIVWRLKFYEEGMAYYATKEYARTDLDKYFQTQKTMDKVTNELVGNDPSLICIGSADFKPNAPIKKYIRCPGTKMLQKYLRKKREVDVLRVDEYMTSQTCGYCHDFFDWGLHDELDKKKQRFRICQHANCQPPQEDPIGKVDKIIAKRNRRILQKENRAYPHIYFPQEHPNQVEPDRVIANGVEFSNQRLNITNRMIAKTNHVNLTIQNGNQKLIWNRDISAARNILYKGLCAALKKPMAPNLDRRNQRQHIAQKREYYAQRRRERELLE

SEQ ID NO: 12 - protein, 745 aa, synthetic construct
MSQKRTCPSLQQVILGTRTNILCISFSQATYRRGPHKEAILAAINKRVITMTRIAYHGSTVALFKCKKALEEGNFAFFNDLKNIRDCFLHVSVAKDVHGNIEDYTRNLHDDVRNCFTEFNIEPPSTEALGNVEPYAVNQYITNFADNIRMHFKKRLKTFFFARTRRNPLLFPDITPQVIHDTVSYMTNALARGVEPNIDLIHEIPNWNQFNISDEYVQQNQGFFEKKLLGTGKDERWFRTVIMFLGMQMEIDDYQEWCRAHPELELGMLKNFVVVPLHKLKRQHIRIDTEAFYWLLRELKLIPETPGKIHKEKAKFMEDRERVKHWYNWFDKEKIRKMEKGGNFFHFQIVTNGLGASVLFTRVNQQSSNKPDNYMESIRQKLESGHYKRVIGMDPGYKVWISAVIRYLRTGQEFNYDVTSRQFHKFTKKKKREKKAKRLTGDFENRRKADAENRELYPAGIPSPNRRENWFDYIRWRFKWCNEGFDLYTDIQYARLDLDQHFRTQKVMDRMVNKLVGNDPALVCIGSTNFNPSAPIKKHIRCPGTKMILQYLKKKTKVDILLIDEFNTSQVCGHCHSFFDREWQDRHENRGRRFKQCTVANCQIRFPNPIGPTNVIVAKKNHRKIQQERRAFPIVAEHPEGQPEQDPKKSTHQFLKTKYNKIRPYPEGPKPKYVCNVQNNDWGLVTQRGNSKITWNRDTSAARNILYKGVCAVLKIPIHPNFVRPPRMNRALTQAEKQARKFKRK

SEQ ID NO: 13 - protein, 764 aa, synthetic construct
MDEWAFWEHQDVGNGQLRLRNEALHRLLYETEFEYPKQKEDEIFTLVRGLAGMLQDEFFFPMMDKINDTVPLITKAIKLGSLHLHFCLFDLVKRGTIREIKEEFLGKFNTIKYFGPTRKTSHQCRYEMNKGFEYYMLAHDVHPPELEYSTQVYAFAVQEYERNFQTNVKTHAYNRIKRLFDYHSGVGKDKYNALKYLFDEDSDIEPNDYVLSLLRVYDEVLQPLTQITPEEKRRRENADEPVIPRGWLATLSDKKKWYKHVYEFINLLQWFYHHNIEEPDFQKKLFIAIPQSKIQNHLITIDTSAIVDMTNSFLEPGKKTNKTTLEGGLDAPGFKDSFWRTYFKIDQLNNGRQYFDFSLQTNGVAVHLKMKKRKLWKPIMKFRSDALPPTSSNIWDTHEFARMTDRMRMRWANKRYRYLIGFDPGYKLPAAIVRQHCATKNEENFTMHWKEFRNRCGDPRRKRKLRKLTGWYEDLARSIRENRELYPEQPSPKSPDYDTYIDYQMRSFNIGCAVYQKESISRLRFDKYICTQRTLHRIAKEITKNDKVPKNPEDKNCIIGIGDFSFKPNSKIRGSVRCPMRPLYEILKLYADVVEIDEFRTSKTCSICGTQMEKTGGRKDRFFVCHNSDPVGSILPRAPEWTSKLHHSKITQIMRRLPEHERYYVHEVTERLPEWRIANLLPDQEEFQVISEIRRQQNAVPKEWTHRNWDFLNWNYDLDQVQHPTIVWNRDINAPRNMIHILKSMLNGIEKHPIFNRGDNNNPD

SEQ ID NO: 14 - protein, 779 aa, synthetic construct
MMVSTPNSTPNPKPKKARQMKDNTFKHYTLVRGLNKMIRPEKFEKLKGAIDTLVPLISKSVILGSRHAHVRLMELVQNAMHDEERTGIRAEDHAEIRAEFTPPAVRNNKGVLQKHGYVVERDFNPTKRTSKQSLIHNDPIHPNFQLYLDVYGVEIPYLPFGVRCHEYAMNQFCTNFHNNIRFHAYNRVRKFFNVHSNNTELKRKTLKYLFYVKPKKKNARQVAIPVPDERLLKLYHEVLRPLLPPNINTVPLGWLGESLRDRYDWYRHVHEFMRLQLWIHANNLQCDEEKDQLPSFTTIPQYNFKRKFLTLDTKALATIANSFERRAEKTPSLDYGSSDIEIRNRIWQRYFKLELLNKEMRHFDYTMTTDGVSAHFVMRNKRKQRTKREILDVEGNVIAPEVVGFIDDRIKREPDDEIRTKYANNEYQTIIGYDPGYKLPVAIVRMDIDTGNETSFTMHWKEFRKLCGDPERQQRRYRIAGKHELKMRKLRENRIIYPEQPSNKSPDYWTYMDYQMRTFNQCCAVYMRRSMARMNFDKYICTQRALQKIAKIITRNERCILAVGDFKFSANSPIRGSVRCPMRALFNLLAQQYGDVIEIDEYLTTQICSICGSRMQKKGGQKDRIFVCKNRKPADLILPRANTWTAKLHKDRIRAQFRRLPEAERYNVQLDNNIIEHRERITLPTERNYQITDNPQRAVIEAGDGDNNVVVVAKHQSIVWNRDLNAPRNMIYKLLCMLNGNAISPVFNRPPPRNNQQADNEQPPLDEVHPQADNEDDEI

SEQ ID NO: 15 - protein, 713 aa, synthetic construct
MAKRKGKFINTSKPKRKKKVFETKETSIKVGLHKILRRTKNTKKIIKEIQERSVKAAKLSVLSSLLILFEVNNAVYEQESAENIEFERTFFNQDISKYIQTRFRSMTQKDSLENNLVYDNFSNLLYNCHIDKPETTAWANSFVYNYEQYETNWSTHINIHAKKRFIKFFKTLDDEHTIKEMKDTATFMINSKSRVSPNENLLNHLITVGIGPDIHIFSGFKRGFFKRRLNKNQWFQTIPAFVRMQRHIDRYNMTKEQERMIRNFAVVPICSYKRRHLRYDGQTIYNLSRRLGILQKGRNRKNIPVNRFTDNATRRSCWDREFNMGYLASLGDRIQRNRRKKQFQYQFTTDRVACSPLYEKSAQVCEPDILNIKINTCIFTLLYISYENGEFKYGVKVDPGMKTYLAAVRQDWTDDNHNFKVGSTEFHRRAKQPDRDEKAAEIVGDLEIRRRLDRENNYPRMPSANGLEWMSYITHNLKFFNETIHTYTKPIYTHLAFDNYSNTQATMDNIVNELTQRQPTMFFIGGAKFSPASPIGKYVRCPGVRKLTMAVKKLPNCGMILVPERNTSQVCPLDLKFFPRQTKKDRFETCYNCQRLPTTKPATRIITKVSKRELQRQRRNARLAEQEANLPQEMNDPQINVHQQPQFERRERRLVSKFTRYVKINQPQPNADHSDNESNDEMDVDMEANDNPLTTVWHRDIAASECIGYVGKC

SEQ ID NO: 16 - protein, 677 aa, synthetic construct
MRQFVIKKTLRSICPDEELIAKIDQLSKNVSEIQTLASHHLYKRSMEIILSEDPERIRREFDRPVIDLMFKKDFFAVKANTRNPEADHEFLQWMANCNPPVRPRDALHCDNALNYAMKLYKTNYGNNIIMRAKQMFRKFCKALIYNTGRVLPQIVLNRFINRVIEWFFYFGNYEPRGFMRSIIDEIIWQCTIDEEDIGRGFFNVLVSERPLNKHPFFYRFAKYFIKMQLYVHHHNDTHPNRRAPSFDVIPLHNYHRRFAMIDTETLRKMTGLHMNKKQPNDIGNFNPRKSDVWSCFYSEDVDYVKDNQRFDFKIMSDGVTANIFMMKLNPHESLNPHIDLDTPSRDEYGQSTRVMDLPATEQFDRIPAEVIERLEEYDVKIGIDPGAKTMAAWTKQYPNLHEECSSIESKTFREMAGFRKHGYNRRSITGRFDRREREAREASDILITSRGPHYAQFVQHKLNFCNDGWQAYHHPYVRELKFDAHQEKLAAMYAVAKKIIGRSANRWNEALIFFGHWTWKANSPIRGIRTPLKLLRFCLQQLGSTVIVIDEFRTSKLCNFCTQTMVNDDYRNSFCVDCQPVERTLAPAPVWRIKAHKSYLKRNPGACKIRTFTRDPNQPFRQNNQNHSPMSWNRDYNGSRAILAKGLDLVAGRPLPRNYLRTTQRVVQRRRFPQQRR

SEQ ID NO: 17 - protein, 681 aa, synthetic construct
  VARIANT 2: note = X can be any amino acid
MXEEVPVEDKTTRGIKCSLYRIIQNRALREKIQHTAKQVSRITILAALYVHFRLMEITLSNNPIDLRNEFDEKECHKNRLFENDFYACYENRRNATHIDLTFQRFMNAKQIRKITVGQGAAQCVKAAYQDFYRNFKFNITTHAYKRTKKIFRCLLKQFPDVLPTQIQAIASDCTHYLFNADSDKVPRRFIVLFFNRFVLPSILNIENHQIYARTKGFMNDSQKTKTWYRFVPNFIRMNHFIIAYNRTLRRGQRGMKSFNVIPQYSWKTKHIKIDTEAMFSFKYPLLHYGHGLDLTNKRVVWDDIVSRLPKRTNYSFDGSITTNGVSTTVTVRHNSAAKTNKYMKKEQKEAIAEANKVAKAAYDRRPKRPNRFFAGKTTNEVPQEIKDRFTHNYYDVLVGIDPGYRNPAAGVRLHMNQDIERNFKFSIGKYRSLQKEWERDNKRKKITKHFDRAEKGSRKRCEEISGVQPSSKDPNYFRINHKLQFFKQVCDAYQHSKISRLALDAYIMKWKGITHLAKTISGSHVNTLNKALIFFGDAKVFAANSPIKGHVRLSQALLWKFLERYANVVYVNENTTKVCNKCFESFENLKRNPSSEYSFCKDCVDRNGVLPSPSRIKTRTHRSYRVKFEYRQEYRIFENFSRAPGHSNGKMYNRDAIAGRSMMYKGICLTADLPIEQHFGQ

SEQ ID NO: 18 - protein, 261 aa, synthetic construct
  VARIANT 234: note = X can be any amino acid
KMKVVCADPEMSDLLYFGSHNYKNELETFRYTQNQRRLETKNKKYNKIIDKVNKETFIDSKTIKELETSLSNLNSKSCNYEKFKLYCIEKNKINNQLYTHYEKSFFRKFKLNAFTNTQKSESKMIKNFENKYGKPNKTILVVGDYDKGDNNMKGKEPTICKKFRRIFRNAGYKTYLVNEFRTSKLCNCCNEEIEKFLEKPSKKPKRKGKIDLCHGILRCKSVKHNCEIYHNRDXNAVQNMLKIVKSIFNTGKRPEVFCRSS

SEQ ID NO: 19 - protein, 400 aa, synthetic construct
MCVDKLRVAEPLPAAKDFRGQARFTLDASGRYHICVASNFTRGAPDDVGTLGQSLLQVERPVLAGCDDGLQEVEEEEEARAKCEEHGGGDMAMQALEEKARVRERKMGEVARRLQAEAREARRRWVRLRRAKAIDKQTQEAEAEFQAARERHAAHLDWRRRLRKRRRLLRMKERNALLRERRGGVVSIDPGVRVPFTLWTASGTVSIAHDAIEELRMLLRNANDIRADADLLEVAVQEGEARGEGDVAAARRRHRQQLRVVRDRVDHLHRVVANYLVMSFRVVLLPRFGTSRMIRRSGRPFGRDTARSMGVWAHYRFRQLLLDRAEREPGCTVVICDEAWTSKTCGRCGRLHHSLGADDVFACPHDSCRYVAGRDANAARNILLLWLYRNDIQLAPVPAP

SEQ ID NO: 20 - protein, 485 aa, synthetic construct
MLSILRQHGVGIPDPPKWMTATMAYKLLNLASKEFTPLLQRDLKNKARAAQRQLKKVAEHHDEGARFACEHTAELALYIGAAQHLARTLAPPDTDLTSFKIFPTFTNKHPFIQLDAGLLRYLTCCKCGRLGHSQRQCRQPAAVRPQPRQAPSLLDFLGADVARQFRKRRVTSIRTDGYTAYLLVEPSARRDTVCDTSPITPVVGVDPGTRIPLACVRIQDGKKMLVSQKRIANQLYTGRGRRHHKPTATVPARSGGSDEGGEQGGAMGKGETGSRRKKRERGKRTKRERSRRRKEKARIEKEEQLRRQQQEEQGGRDKGEGQEGRDKGEEEREQREREEQGGGAEPGSVDDVVASARECLREGVSRRELKWRLHEKLKHCRFRSRVAGELATRFGRDCRVFFGDCVHKGRGGGTKAPILELRRAIGCRVDTKIVEEFRTTKYSLCCAWPNRIDEQTRIATCTHCGASADRDVAAAKTIAARYGSM

SEQ ID NO: 21 - protein, 633 aa, synthetic construct
MPPKKKQKLEKLDKPTCLHTCNKTSFAKAFLPNETYRQRLLDYIAIIHQLADHASHALKFYILSAPTFPTVNEDTIEAILYLLNKGEAWHPRKEAKKAWRDCLLPYVQRYCQIVGFVHPNLRGEQQSVNYLTASMMTNLKVNVQEHFMQMLLRYINLRLDVKGQKQRLPPKSDARKAFFARLRYLKSVFLFDIVPESLDDLTAEESELLEEMWTLNLPFPDQDQPLAYLVAIDPLAFFPAYCRLSRLYEQHGFRRFSAIPLRRSLIQSHVRIDTVILYPHILCITRREAEAVEKVDLWLRVCNLRTKAFRSRRGMQFEGSITTDGTSVSVYLKHPEADKYGKRGARKSAKTLEAEVKALYVEKNLPACRAAENVVVIDPNKRDILYCQDSNGTTFRYTANQRAMETGSRRFAKERQQMKKEAGIDLIESRIPSHKTMNLMDFTRYLLVRRADWDRRKEFYSHPAHTRWKWHAFINRQKSESDLISNMRNKYGENFTIVMGDWSDAGRTARFQTSSKTKGWRTLFKRNRINCFLLDEYKTSSVCPRCLASSDDFLEKGFKTRPHSRPWRRREGKIEKVHGLLGCTNPNCLQQAWTMRYWNRDTLSTCNMLMIVQSMLDGHGRPEVFSRSVPAVA

SEQ ID NO: 22 - protein, 663 aa, synthetic construct
MLSNIYKPISSLMNNRSIQYLKDELPIFPQSSSSTMPPKKKQKVNQPAILHSCHKTSFEKAFAANDTYRDRVQNYIIVHELAAHATHALKFYILSSPHFLIINETVVEAMLYLLNKGAAWQPGNMAKKDCRDILVPHIDRYCNVVGFTRPNLKYDQQSINYLTTSVITNIKVNVQEHFVKMLVRFINYRLDVKNQQKRLLGAERMSFFARLRYLKQLFLLDDIPQTDDLDLSNLESALLEDIWNILLDRVIGKEGKLAYLVACDPLSFFPSHCALSRLFERCGLPLFNAIPLRRSFIQSHVRIDTMILYQHVLKVSWREYDSVDKMDLWLRVCNLGSKAFTTRSGMAFDGSITTDGTSMSVCLKHPDAAKYGRHKHRNSEQAIEADIKSQYIENNLTACRSASNVVTIDPNKRDILYCRDLQKRRTFCYTSMQRTKEIGSRRFKKSREAMKRENGIDVIESAAPSHKTMNLDSYIHYLSWRHDNDAPLKDFYSHPAHTRWRWHTYMNTQRTEAKLISNMRKCFGSSFAVVMGDWSDGGHTQKYQISSKTTGWRNCFKCNRIDCFLLDEYRTSSICPRCLSGDHLQKGFKIRPHSRPWQRQKGRSEKVHGLLGCTNPNCIEQGCTYRYWNRDKLSTSNMVTIMKSYLDDGVRPAIFSRHVPLVA

SEQ ID NO: 23 - protein, 628 aa, synthetic construct
  VARIANT 569: note = X can be any amino acid
  VARIANT 571: note = X can be any amino acid
MVAAFSPLTHPDMGCPTCNGTDHHRASSKLCSFYKPRKKNEVRTTLKRKRDTDLPNDEIFILSTVVKSGQRSPLQVVIAHAARTLTCIAAEATRLFNMFVLDLLERKQQIPECSYNKFLHQPFQIIALAKASSPQLQTIHRTLYRQTSRYDVTYLSQLISNMAKDYETNCMNHVSTNIRKWAFKWYRMKMQKTYPDLKRDKIRILFNKEDDLNEHSLYLKACLMTYDVDITKEVSFDHWWTYFPLMWKFQRYFAKHGERNFSMVPITTYGIRHIRLDTNALYKLCLRVDLFTRKDVPAAEFRTNALVYWKTLFPDIAETSNKSFAYGVSTDGVACSYYYQKRYVRRRQSHNYYGFDDDGKYGAMNLEDGVSIVGVDPGRIDIFTAVDGNENVVRMSRKEFYSLTGMVASQKTRNRWLKNNPAVQSIVTQCPTPRTPDIENFRQHCQYVQQHLLTLVDFYGARRFTRQKLHTYIGKQKVWATIVRRITKNDRRTVVALGDAKFRHNSKGGPSTPLARMAKELKRRHRVRMIDEFRTSITCSHCDGELPKSTRHWQVKVRWAVMNSVSQKXSXICVFCQVCNDLCLTHWNRDVNAARNIRNIFLHMNENNGERPCCFARTVPQFNPGWNL

SEQ ID NO: 24 - protein, 928 aa, synthetic construct
MDRNVTDHKIDIIIDSFELYVEDNSDDDFQPVVTARKRKSRENIPQSGSQKRLKGKGKATDSEVTTTSSTGISTTDEPTVFNKITLEDYDLEYSSDDFDAPVIKRPFLGESTKSASRITKSTSAAVESSTTASKTSINSPKPSKPVPTAFKSAATSVKSSFCPVPVDKASDIKEGPKKISKRVMTIKTTVKNIWKPAYLQPLYDLVHTTNLLVTHTFAFTKYIYLQELAANENFALNNFVTKDFFVEVFLSLVLSKGGNSTRLKDTTKNYRRLISKYKEAYFEDAGYTPPNLPYAQQIALYECTKIQTAYYNNIKAHFGNRLRSLINKLFKKKEKAESLRGEMQANKSSSKAIKEAIRKKIYRPCNQVKLAIAKKEMPEVGLLDDQSRTQLNDFLSSYPEDYTFQENSIYYDVMASPKNHFKAFFRLAELSEAEQTKQFACFPLRSTFIPCYMTLDSKIIHYHILKSKKNPKTGSKFETWGAVVDLNKKAFKHQGFQKSLRFQGTLETDGVGVSIIKQNTDTSRKSPKPNTEKKVDGNQTEHIEGLGQADLKSTEGKCVLIDPGRRDLMYCMKETSTVEEKQTLIFTKNNRSKCSRHFRYLRKRTQPFVVQKAEAILSRSESNSVNLKKFVQYIKTRASVKNTLYEYYGNETTKSKETYFPESEFDFRVDQKCNLYYGNLFIARIRGFFPQPENYSTDSSVKSQLYATYLQIMLQQKHISERLDDSEKSKVLGLAKEMYRRPQKSNYKKTISTALEKLQLLPFRKLKFSSKLFFDQNDQKLVRSLRAKFGQDAVLVFGDWSAPNVKYQEPTRSKGLIRMLKKNGFVVYLINEYKTSSHCPTCENELEKFKTVPNPRPYQRKKKPDVLCNGLLSTRCKSLSCLKQQTTGIKRRLWNRDQAAVLNFLKILNSLRKTGERPSLFTRKYPLD

SEQ ID NO: 25 - protein, 690 aa, synthetic construct
MNEDSLPSTSAGTMSTTGTLASGDRILSVKSSIDGIGWNEQYRERLEEFVNIIHATTTHAYSLSKFIFLCALQDDANFDIASYINKEFFSEVWLSLVRYRRGRVGEATARRRTFIGQYIQRYLDYTTYERPELKYAQQSSSVEGLKIYTAYTNNIGAHFGNHFRRAINTLLQIRQRKADLIRQRQEEGVDNEAISNEVYEQITLPARQFKEILASRIVDPSSIELPFQDPIYNQALQRLTPILTSYPVRYTFEQNNIYYDAKANPCEHMMAFYQLARLFEALRLPIFNCFPLRRTWIPGYATIDSKILCQNILGRRWTNGINKMNLWAEIIDMNSAALKPQELGVLRFRGTIQTDGVGVTVLKKRQDRKHRYIGLQSTFVEPEPYITDLNAQQHGEITGRCVTIDPEDETFCTVYMKTLLLMHHAHIDTPSLVKIRWKKTKKYRRICEAVKPQEVQNAEDTLVNSRSLDLQNFEEYLRNRALVTELLQRHYTETTTNHLTTHPLHRKLKLSKYIRRQKASEDMVAKLKSKFGNDAVFVMGNYSAPNTRYQEPVRGVGFRRLLKKHGFLVYLIDEFRTSQCCPSCEHRSLTTFKRIPNPRPYQRRNNPEVICHGLLRCTNQNCKVTVQNISGAEELRERLWNRDLAACLNMIHIVRNLRLNGEIPERFQRAGAERRRSEENEERRVLPRTL

SEQ ID NO: 26 - protein, 720 aa, synthetic construct
  VARIANT 650: note = X can be any amino acid
  VARIANT 720: note = X can be any amino acid
MLSKDNDSTNDNSNKKCCQFIFFFFFMEPPLKIITVKSSIAGIGWKPQYIPLLKTLVANVHTLVSHTFSFLKYIFIHELENNPAFPLENFINVDFFKEVFLSLLNNYKPDKQKASTKSRTYKGLVLNYRHQYFQCCSYEPVNMKYANQIASYEVVKIVTAYLNGVKVQFGNKVRMFLNLLLKKNERIKALKSEMKKNGGTEKEIAATIKTITEQISKVKLAISSRNIEDMPKEFFSSNDLDKIRNLFDSYSTDYRFAKGSIYYDCKANPLKHIKAYYRLSSMCEALQNKSFNCFPLRKGFIPSYMTIDTYILNTQILKNSIISHLDKEVVWGAVLDVTSKAMKPQGERKFMKFRGTIYTDGVGVSVLKQNYDTKKKGGSSGGKPKSIEADEFQYIEKLGKEDLLAGVGKCVLIDPGRRDLLCCMHEKSTVENKMIYRYTSNQKAIETKSRKFRKLRNNLKRDEVIAAELSLSHFKSSTVNKDKFVEYLQERAKVTPVMKAYYLNEDRPAAEDQGAGGFLPFRKMKLSSFINQQQADKRLAKKLRERFGNDAILILGNWSAGNVKYHEPIRGVGMRRMLAKEGFQVYLLDEFRTSSLCPSCQNGELETFKKVQNPRPYQREKYPIADRHGLLRCKNQQCLKAVTSTIEATXKVPLRRLWNRDMAATLNFRHILFSLRANGERPERFCRSKKPLSTGSKRKNMSSSSASTSRPTKRPNNPLX

SEQ ID NO: 27 - protein, 1165 aa, synthetic construct
MNRTKAKQSSRFVGTSTLPPRRKHHSNSKRPRSPSPGEGTSDNKRKRIQFSDTELDAFFFLSMENKLKESIRTVVRKNNKKMNEQLAALKQKLSTAKKLPSGIITGAAKLAESTYNEPSQADLILRQMRSFMDNRVGKKNEIEWQEFVVKKFDTFKNEETNLAKSAQPESNDTDRDSVPDADCPAVDENSYRTCTASINKLIREDLDPSIKDLIMKKLQRAMIGSTDYILYFSSLIQMMMICLKTNQFFLDGDVINTRKVNGFDMTTIVPSEFHTDSPKYTITPLAMDVSDSPAFDADFSQLFQEQHFSLISTKYFGNRGSQDQTLNAHPIQTALFKLMDAEDIKKETFPVENVPSSLMSLALQIYVTNFKNMWADKTMINKLLDKLLVTLLRLHLAPKRELQRRQNILNYISRKPEEKRPSKNYGRYILRAEKKKLARYKEKKQDASSSSREKSVESTTTTKKETTPCDDLSRGRISQLKSGAKHLIFSKDLVTEKDVNDEFPDATRKEVSVCLLIINTLNKFVPVRSKYHILPYQLLFCIFANDVLHYAEYSKFTRKLCPAMSSAHVNALNIDAPSLFQIMKSKTDDNQGQVEGMSTELTIYGYNEEPLSSVEIARRNKDAVFNSLFDMEKVRHTCESNGLKFGQRMSVLPKLKVVRILGVKKTTVPPVKPQKMSYLKRILRDPNILREGQKPLEDLKAETAKLEKEVKSLDQEREKALKCQKEQDSIKKIKALRKEWHNKVDKQTLYQRIEEERHLRDKTYLKINDIEDQLMEKKQVLYYQRMAIRHNSRIHTVDTASAPPTTKKELKKDSRGLVHSEACVLRDFEDFTFCGTDNGLVNMTAAVPFSLKRFSFHLKLHNYYSVLDDDNKAAPVVQDIELQKYLKTPKATTISASEIDFGCGAFKQRVKRQTGKKLTRKGKEVQLIENSLSQKVTSTSNVSVEQFKHNFDAHVEHRKKLRDFYNTGNRMNERRHREIQKHRFTHRICTKERKALSQTSGNNPSTKKKLVMFIGDRGTGVGSRIKGFKRYGGRWKEVIHSEAANVCVTNENLTSKTCIFCFRRLTHPRISVTKNGEVFSRKVKGSFYCTNPLCVSVLSKCAAKSRDSLSALAIGIVGLSTILFGAPLPAFKSHKISNIEAEKYIILTSPSRFEEETGSYAVVLE

SEQ ID NO: 28 - protein, 871 aa, synthetic construct
MADTTLPVDAVPSDAAPVAEPILPAPVEDVAPGLAPSVPALSAVAPSDQARAPIVSVVPQTLHHALVDAPEYLAVRAVIEARSERLVNAHGFAATEATLYLRYRARRAYEDFRDALLYAEEDYESSNDMVSLLKAVTVADQQADYLVFGIPNCDQLDQYATEYLHHVHYPNTPFVNRSFFTRSFEYLFQEIVKNGSEHFHRTFRYAAGRCINPFAVLVESWPPPAPPRDQRAVEPPANRRMRRDFYAAIDKWVTTICRRGSKLQLDKFLYKCACIIVDYNLFILQNPEYARATSGLVPDLGVLHVPVPFVPPPPAAADDEAQGQVEQQDSVHLPRFIPEGVRIAAETLPRNRHARRKQGKAWIAPAVADHFAVARRWFCPDLTIFPHMALKRRHVRLDSMDITRGLLAEFPGNSPDLLHVVFGLIMKHAHLDSLVPGASDLNVPDEQYELALEQYAWQKLRDISPQLARLRTKFCSDPSISPVLSGYWQKPRVDGAPRVGSSFTTNGYAAHILISRVPTDDDVRRVGGEGEIKQKSTRRVVTRGPGRAVAAKSLHSRLGTGEFAAIKSRSLIVSDYPKGLIAVDPGQSTLMTKTTLTPFRVGAFANGWINIKWEITRRRNPGYKSWSIPGPRWSAATGQRNQTLQILRLRQHQSDLEQIESRLPAVHDPTLAGIIVLAKAVTTNMERILGFKMDRRYAKYDLQATIAKQRALAYLRNKFTGYGVLFGNKGAVQINRVTRMPVKGVIRYLLVYGIDVLLVDEHRTSKFCAVCGSEFEATPAMKVNRKFAYRYLHCPSCKYVVNRDVNASWNMFRVALRDVFDIYVADSMLKHPLALLPSRGAPRPLLPQPGDDDFDDSAHDRYSGNHGGPGG

SEQ ID NO: 29 - protein, 645 aa, synthetic construct
MGLVRHFTSAVTHGDVRIMFDVAYPLKDRGGDMRIIYRIVIVASTHDCQVAVAGRTSCVFDLATRSQASVVLAVPSSWIVSVLVRLPGKAMAQEMPWQALVSLDIIFGLLSEIRIAKFGHGVTSAWIKRILTVSGIAKGLIEVKSGNIEDDLLHLSDLSLPAARMFTVQAMSNDYWEIELPDLPSCHYLTISNSALHFVTDMMLKGFLDLTSHLDSMAIEQPIALPDPVLDQVALTDFPHRFRSIKRLHIGVTAYGWFVSLDGDHEAVHMTDLCLGNDYGHGVCCQRPGCTEWLHKCKAAAQIQCHTHLEHNACLDPVGQTVTPNWSRVLTSSIHWPLSYLMKPGEFELNHCQLQAMLNNQLHTVPEVGANAAAIAASRRDAAQHNQLLETYVKKSQHTQAPPNQTGKWRSMSKGMMQEKRGRVERSQFMDKFRSARPALRRFNANMPMFKTLDLAALHNACQYVTAWLPVVKPMALSNSLASYEFGAANAERAFFDRLAAPLMLSPDPTRPALVYFGNVGHLSPHPHGVSTPVRKFLCMLVHAARVVLLDEWKTRKTCSLCGSEMPNDACRWGAKRCRTCKRIRARDRNAYLNMVRVAVMDVLAVSFPQHWRDRGHPLCKRAPLVISLGGAQQVGAGGEGGGAA

SEQ ID NO: 30 - protein, 789 aa, synthetic construct
MHVCMQLMLKLMHLPPKFHNQDAKKMQDVMLVTTLELAEALATGRVMAEQAVVAFGHQLLATHMQTNCLTDVFLKEVLARVQHLDATYQVTGKPVGKLHSLLISIKDMNCMAGTDMALSYSAFINQPSQSNVLMVDIVLCEGANIIAKTNFLQMMLLFECSNPVFGHTANPYNLNFMLGCTPDAMEPSTVHTPDESSTVHWHVYTQWVQCPHPHVIHTACKTAATIKVTGHTLVPFTVLALLKLMQITYNLLLQDNAHATMVLVEAAKEPIELSAIKLVQLLQLPQWVWNVLVRVMSWLGEMCTVMFLNAMCIKLNHIVPKRLLLQYLDAAANQLLVMWHIMPVPTPQAATMSWQWLGSTTCLRVRTCTACSHMSCTTMRLSTMGWARSVLKSCMPWSLSCKMCSYNLLVEVVLDLNMVHWHADMIDAVLGEAPEHVDLANLLMPKDWFDWAMAENVAQGNNYLARQLVNNARKLRDEVRACGVAAMLKDKYQKTAPNWSEHGPIVGVESRRQGYEQRNLDPDDFGMFRFVDPGHKSVVTVLELNHNELQAVIDNPLRAMPEDGADAEAIAASESDAAQHNRLLEAYCPHSQRDNSHPRYEQKFWSISGQQLREKQGKTQCSRHQFRAANADRAFYDRLAARFMDSPDPTRPALVLMGNSGHFSLRGVSAPIRHMVRYLIHAGARVVLLDESWSSKKCSLCESDMPNDRRRWSSKRCRTCKRIRTRDRNARVNLIRIAVRDALDVPFRQHWRDRGHPLCERRPHANGVPIGDDGGLDQGGGGGGGGGAV

SEQ ID NO: 31 - protein, 683 aa, synthetic construct
MNEATPFRALTVKASIKATGWSPEFLRPLQVLVAKFHTIVTHTFALSKHIFLAELALDPFFDLNGQINQPFFVEVFLSLIDRNTDSSRITEITRLNCELISRHINTYIHNSSYTRISLLNAHQIAMYEATKIHTAYIVNIKLRFGQHLRRVINILLDIKNRQRELTQQLRSQRATEERIKRMLTEHIFEPARMFKEGVSTRRLEDVFIEEGYRTAFMEISPILTAYPKRYKFQKDSIYYDSKASPHNHFLAFFRMAQLLEHLGRRSFNCFPLRRSWSPGYMQIDTRILDTIQQLLRRTWSEGADKLELWGIVVDLDEKAFRDQQNGVMKFRGTIQTDGVGISIIKQNKDTTQGPNPRRATTINPENIPYIHDLSPEQHERIRGRCVLIDPGRRDLLYCMHEESTVETPRCYRYTSNQQNIRLKRTKFRRILQDLKAQAPNVIAAEHEISQTSGRPNNTTDFAFHTSVKAANSSILSSFYTNTVSRTSQKPLFRKLRLSSYINQRKANERLAKYIRAKFGQDCVLVMGNWSAPHNRYHEPIKGVGMRKLLRKHGFDVCLIDEYATSRHCPRCYQPSLQTFKQVDNPRPYRRTQMPTVTRHGLLRCTNLNCLENTDGSEVMHRLWNRDVAAVLNFRHILNNLRYDGTIPVRFTRVIQIGRIRRQAEEDLQEGRRLRQRLTRIQRR

SEQ ID NO: 32 - protein, 677 aa, synthetic construct
  VARIANT 19: note = X can be any amino acid
MELPLKLTVVKGSITSIGXKLQYVPRHMTLVANFHPLLSHTFTFLKYIFLHKLEDEPNFPLENFVNEEFFKKVFLSLLKNYKPTKQKASAKSIAYKDLIFSYRDQYFWCCSYEPIDMKYAHQIASYETAKILTAYLNGIKLQFGNKIRMFLNLLLKKNERIKAMESGMKNNGENKEDIATAVKTIIDSINKIKQVISSRNIEHIPGEFISSATLDMIRNLFDSYPPDYQYSKGPIYYDSKANPLKHMKAYYRLSIMCEVLQDKTFSCFPLQRGLIPCYMTIDTRILNTLILNYSVISHLNKEVVWGSVMDINSKAIRPQGEAKDVKFRGMIYTDGIDVSVLKQNSETKNKGVGGKKSISLVENEFRYVEALEEEELQAGIGKCVFDDPGHRNLLYLMHKGMTSKRKSTYRYTRNQKYVGTKAKKYRKLRNDMKLGHIIAAELFLSQHNSSTTSKEKFINYLQEKAKATPAMKAYYANEDNPAEEDQRISGLLPFRKMKLSNFINQLQADKRLAKNIRKKFGNDAVLILGNWSAGQSKYHEPIRGIGMRRILAKKDFQVYLIDEFKTSSLCPACQNGELETFKKVQNPRPFQRRKYPTVDWHDLLRCKNQQCLKPVTESTEDNTQSSLYRLWNRDMAATLNFRHILFDLRKNRRRPERFCRLSARSFTTLKRKRSSSQ

SEQ ID NO: 33 - protein, 1186 aa, synthetic construct
MRRKKTPQETFYQGVNTLPPSFLQSNTKRTLSESFSEESFNKRNRVEGPVKDLEECFFTSMRDRVEELVKQTKTQDKTNELKNKLLNVKTLPAAIITNAAQLAKCDRKVMRSHMEHRPGLKNVEEWKKFVSQNFDKIAEAAVGSVVKDTAKVYSSSTPTNKQPYEEMRTCTVQINSLFRSDLPPIVKTFVCTRLQDSMVTSTDYTLCFSALVNMMISELRTSEFFFDNNDIKIKKVPGFNLAELLPFVTTNEPKQTIQPLDKDLIASKRFDTDFKCLFTSQHLQVVHSYFFGARGAKEENLNAHPVQNSLFCSFKESGLDKQSFCLEKASSSAMSMALETYLVNFENMWDGKKIINKLLDKVVLVLLRHHLARNRESKRISTTTTRNPPGKKDMRNHARYVCRAEDKKLKKLVQRKEKAGGLEGEKWATKINSAKQRLANLRHTFKKKISQMLNDRKEASVEHKLVVQDLNIAEEQQDFLEEEGTLDDDVPERRLNQLKSVIKHLVFSNDTPVYLEDVKHQSPEATTTEQSVCLLICNTLMKFLPPKKQYHVIAYQMYFCIFANDVLKYARYTKFTRALCPSTSFSSSSALHLDSVALYQLLTQNIDQEKSEEPPSHTNQQEKKGYSRMILYGYNRDELIGSQDKARQNKDATFNAVFDMGEIQKACESYGLSFAHRMTCLPGMKTVRLLGSKIKTHGTVKEGTKQSYEARILRNPSIMQEGRKTKDVLFSELQSLTEEVKTLESVRKRELDLLKDSNFQRKIKECKSNWGTTDDKDQLYRTIEKYKESRYKSYLTVNKIRNELAEKRQQLYFRQMAIRFKSKLHNVKDVPQDSRSPIEKCGKGVTKAEDRTVVNPGDFNYAGTDNGLVNMTASIPMSLQRMKFHLKLFNYYTALSKVSNEDSIGLNLSKEEESFLHLPSVTNTKASDVDVGCGYFRQRKYLERRKKYTDEGKKVQLIEDSMREMESAPVTSIDMAIRNFRAKYDQRRSLRDFYNSPKIINQKRHVEIQQHRYRHHLCRRERLELKHSENKSSSKKPLILFIGDRGTGAGSRIKGFRKYGGKWKQKIHGEAVNVCITNECKTSQTCIFCFSPLTNPRIPGKKEGSYKVNKGTFLCINPRCITVKNRCASKPRDALSALAIGLVGLSSVMFGAAPPPFYNVSQITAEHYTKITSDFCTRRDDLAATL

SEQ ID NO: 34 - protein, 686 aa, synthetic construct
  VARIANT 139: note = X can be any amino acid
MDDADFPGGNGDQQGAIDAMRGERITVVKGSIASINIRRPVYRDRLEHYVNLHHKTTMHTYRLLKYIILHRINNHHFDAMYYLNHRFIHEVYMKLITKARERAPRTQDTIERRAIIDQYLPAYLRLVGINDPRELPLMXNSQQSAMYEAAKISTAYLNNVRNNFGKRLRQVINVLLNVKARQRALRQLLRGQAMDQRAINQAIRRQITNPARRFKIALSNRTTIEALHARFDDGPEGFYTTAIDQLAPFLETYPNNMQFAQDNIYYDCKANPHLHFKAFFRLAELLHQRQVRSFCVFPLRQSLIPGYVIVDTKILMTQIFQRSVRPGEPLRHRHEWGQFIDFRMPIFRAQAGREFGNMIETDGVGVSVLKREQHDLQFQQPRQQGAPQQQEFPYITDPEVQIPPNCVVIDPGRRDMLYCMEENSTPQAPRMFRFTKPMQDKIRKNKRYRRILQQMKPRRIADMERELTNSNTLNLQVYQQYLQNFGRVYEALLLYYSITRGASQTGQFPIHRKLRLSAVINKNRCDQFLIRFLNTKFPNTTTYIMGNWSAPHTRFQEPIRGLGFRRLLQKHGKQVFLVDEFKTSKVCPQCQQPTLETFKQGINPRPYRRATQLYTTVHGLLRCTNRNHAAHGLPQRFPFRYFNRDTAACLNFIRCLQAVRQGLPRPLDLQRRPLQDNDDDDEEDDD

SEQ ID NO: 35 - protein, 1375 aa, synthetic construct
MHSRAIGMVHSNPKRLSLSKKRPSTQQASFPKPVLPTSSLSAQAPSAEVISTKSGGINVPSSKWSPKRVSGGEARASSESAGSSTGVTASSSRSTSKVADLAKVSQTGGAHVSDDGSSSSRVPPDQSTVSGGGGEIASSSTSGNQESPSPDAVALRVPFTVECDLLIDTNDLLPQAAVSGIQKAFDDAQSIGFNESINNCTKFLASKHVVIDGGHRTLCNVLNGVSRTECKIHDNQGGSQKPKDMLLLYCLASHLQCAVYVFSTRHRPVLVSPSAQEQVQGYMALLVHKDSYMQKTHWYPLRLDLKSKVGSARAKTTLETPIVATRKQSIAQASTVSSSKPIAKGYEKDIEDILRASCFSIFRKKWDDFRSSGNTSIQDFKATLSVKSFPENVVQRCSDAIENKMRAAGTPLSRNPQWLSRKLHSWKTRKKNPFGVVQLVEEFIAIITKEQNKELADQGDDDPIEVEDGTSQDQDDPIEVENDISQDLDDPIEIEDDTAQVQGQLVPIKEYHTISRSLKSVIKPQADYQTLLLRMTNLQNTCHHVVYGLSQCVSTLVNMIIEGECSAGAHPGYLDFSRIDLHQRGIGAGAEQAISLVAPIKQDVLDLFNFAGFYKIACAVVGQKNLTAHSADFPLPANDRVTFVCGPWVQDACRRFLTNFINMWSPASCVADLRVVMEMLLRIHLCSQRFFNPKVRPKKTKKQNKVKIKAPSKDYAMSKLFWALVHKDPKQHVVDFFTTKVFKLFPTPKDITTAATVSASASVATAMNPYEKALQDGTDASEDEEEEDGILLAQTPDLPAKDIRALTSVACFLCQSPEHPTVITMQHVKEKLYHDKQDCIPSSALETTASLVSAVRSVTPKEDQAFSLANWIPMRALRNTLVEFAGSKTKRRVSKLCVDKKKDTNSADAVRIDHAALYHLFSNDHNLHLPNGIDKVTSVHVLKTTEQKVDLLNNFLKINKIKGLFQERKLTFAGYFLFSNQYDLHFAGYKIVQPVKQADIATRGRQERSGNQNHEAEKQLVKELTKEIKDMRKTYTANAKELRKLELARMQAGNKSRTFERRNKRRDPNLYRDLKDARDTFNKLHAKQAKLLQEIQERQSRMYILNKKIHNQRHPPQGSHLDPKLAPIVAASSTVVQGIDPGIVTAASGVGTYSRSLFEAVNRFQAISNEEEVPHPKDQAYPFSLTANMVNTAVLSHQHRKQREKKDIGIKKQQRNTVIRRCRLKKYHQRKVASDNAVYHQPKGAHIVTFTGNWSGSGAYIKGHARRSTKLYYSQLGAGTNHSVVSVDEYKSTVTCSSCFHRTSKQVHLRDGHIKRIPGAVVCLNVKCPRRLTSGATTVNRDLNGAKNIALIGFSSLAAQDGLALPPFRRSHNTN

SEQ ID NO: 36 - protein, 1304 aa, synthetic construct
MSMEDTSAIFDITCASPATLQGNTDLAFYDTMIAALTNWGPLGCKESFKHTAKFLRSWGVILNENGSDLCSIMNGTVKHNMRIKSEQHPESVSPKGFLLLYCLAEHFNCTVFLFSTRKKPTIIKPMQGDRATRNFAALLCHQDSFMQKTTWFSLKRQDGFIRNSSALKAPSSLANANNDIVATKFVKDSKKARHETIYSNEKSDQQVMKHLQEEVFSVYRKRWCEFLQKKQSSKSVDDFRKTLSTKSLPNKVVSKVQVQMEQQLGNIQPGWVQAVIQRLRTAKTMPTAAQYVALFIKDLKKELETAQNGEPSTSGTSASGTSASGAITAGASKAGASASSGTSASGASTAGSSTAGSSTAGSSTAGASTAGASASSGASTGPEDGAGGAGTGGSGSDQVIRSVSRSLKAVLHPSFDSKVFFEVIKNFQASCHLCIRGLSKAIGVLIDFVLRGQLGGKTKATFFNVEDVDLFNCGLGKKAKPISFINDTDAGALQGLFDYSGFWKTLSFCIGRKNEDKTKIPKCRLLVRIQEKLDRTDYVDFTTRSALQEACRMYYTNFSNIYSQKNCQAVLRTVLTLLLKINLCRERLFGDKKTRTYKRKQKKGPKPQSQQATKKLAMDKLFWLLGSDCQVDQCIDKQMRIIFSVNKKKPNSKQLAAPSTPITPSSVSSKGKSPMQATSSSCSLSTANDDPNIELVDNDDSDLESVIDDDIQDEEEYRDLTAKEINALTSVSMMLCQSTTIQGNVSAQYVKQQLYTDKRDSIPAIAIDHCCAIINAIKSLVPKEDYPPSYKWFGLFELHNTLVTFHQGITRKNPRALFPTVDNPHNSQDRMAIALDAATLYSLFCKDYRIYQKDKSEFTSTGMIKTTQAKEDLFSNFFNMPKIHKILREQKLTFCHYIKFKDLYNVHLVGVKTLGSSSSSGIKSTIDKPDPKPHVPKSSRSSSKADVIKANTLSSTLKETKKKLRPLKLQLSKLQRSRMDAGHASRSWEKTNKALDWDLYSKLKEARKKHDTLWATVSSMKAQIKQMSSDLYILNKRIHGQQEPQAADAQTPQLTIDENTVIQGTDPGVVTTASSVWCSSRTLFASINRYQVLWDDHDTKPPPADQKQIVKSELTAKKIDTAVLSPRRQKRTRSSGKTLQKARATRRIRLKKCHQHFVAAEAKSADIFSTGQKPRRMVPFIGNWHRNATHLRGHCTRSMKPYLSNLSTTSTVAFVDEFNSTKICWACFHETQKQLMRNQEGHMTRNLGAVTCYNTKCPMRASKQTTSNRDAQGAINIALIGFSKLVSKDGSVLPPFARKTNHDK

SEQ ID NO: 37 - protein, 1245 aa, synthetic construct
MSRRSKPVQQQTTFTGVNNELPQLRSRESLKRQISDVPEGPSSKRTRVHSLRDYFNQSMVNHLNSMVQNAWKKSKGDKAKFTKEVNLLKAKSETPRLPSGTLKDAANLAHCEHHEMITYMDNNGEKLNKFLWKDFFDANVDNVAQRFQAGHAATSSSTPAGPSAAPLQPMAAGPSSAPFQPMAAGPSTPSMPFTSPFQSTAAGPSTATASSDDDDTSSETTISEIGSVFDEDPTKDYRTCSVTMNKILRADLPEAIKNIIFGKLNDATHRSTDYALNYYLVLHLLLLKFRDGTFALENQTPVFRDAPGFAIARLLPAGYVKSEHCNVTHSALPIPTAFFASTNLAQDFDLLFQKSHLALMQSYFFGVRGALESSLTAHPVQKALFQSLIPCNVTPQKYSAHGLSSAECQNVANKIQTNIGNMWKGNKIFKHLLNKLLHALLSIHLAPDREAQRQEATTKRDKGKSKDKGKGKERATPLPPSAGGPSTTQSASIKPFTRNAFRNIRRGETKKINSYQAKKEQDPSNPKWDSKIQHCQSRIDRCYHLYKNSSDLSRRRLRVLTAVCRDLLLDDGIAACTEDHIKKLLEDPQQPEVDCCLNLVTRLKPYIPRKDAQNSLARVLPFCLLANDTLGFTGYNKFQRKLLPDPQFTHQYPLSLSAQTIYYALDRGPDRLIMYSYDGSVLISATKANQNKDACYNSIFDLNAVQQVCKSYGLTFAQHMTLMPGLKTVRILGCRPKKATYNEDTKGDKQQRWTKEVLQNEIVVREAQKDDDAINADINEAEERVKVLKEELKSALKEFDYASFGQKIKDKKKTWIPGVTNNALYGEIQQLKIDRNEAFMKTQHIRQEMRSVRQSLFYMRTAKKNKSKIRELNAAKSTATTTAEDIPALRRGGHGVHIGPRRLAQDIDIDNDVVFSGTDNGFVTMTETVAFDAKRFAFHLDMFCHQGLDKEEQAALVAKHDPAFLSLPPSYKIKADDVDFRCGYRKARLKLERRKRENPHIRQLEQASRESSLKGADTAQSYLSRFSVQQSNSGALQAFYDSPARRAENRHLELHNKTQRNKICASERQFVAQHKKEESSGPAGEGSSRRKPKPKPIMMIGDSGLGVGSTIKGYRRYGGTWKQNLQGGNTHVCCTSEYMTSQTCMYCFSKLTHPTSKQVVKGKVVTKKVPGAFVCLNPDCVLQQFGRNVFARDKLSALAIGLSGVNQLLFQATFPQLSRKFSDTSTDFINKTASFRSGREARASV

SEQ ID NO: 38 - protein, 1212 aa, synthetic construct
MFSRRLIESSLMAIKRKRRYQETTFTGVVNNPPKFRPAGQRLAERAAVGRTKRRSGDLTEDEAWRFFDDGLKDVIRSQILGARLDERSIHDVRQKLVSSTRILRNVYSNAAKIHDLNAKAVTDKLRYKNTVKHQQEYIEDHFEEIANKPDSFPKVRPQPKKDIPGAPAGKQQRTATAPSSDLRTVTTTFSSIIRSQGITNNMAQHFREIFNAATEDVSNHIIDFSVQVLRYITMFKTHTFVIRNDQVQSVPTVAPFTLAEILPETIFDEATLERPLPPPLDQGFLNNAPLLEQARNFLNDQHLDRIFTAYFGRTPALQVLQRYPLHLAVLQTLPPPAEAPPIAFSHAKIVARNKFKTNFENLWNLKGRFTKVFNKLITTLLRFHLAPEREAKYQEYLTSRTQDQGSSTPTPAQAPHGEITFAGKTTNEIRRIFHHERRNQQNYTDKAKTEGARWVQKAQRSAHRLATFTAVRLAENGDLSARAIRVLVAAMRNVIFTLHGQAGQVTRAQFRRIAGNTINGNTLEVCLNIVNRLMPYIPSRQNWFSKLHQIPFILLANEVFRATGHAGFAVKLTNLPSTSSLLALELDGPSLYNTFSVQQGDRRVLNLFTYDGRIITEDVTARSNDDAIFHSFFDLEKINNILAPYQLQFRHQITLLPGLKTVRLLGSKRDNATVAHAALPFVEKKATAPSKPPPAREQPTDAEKREIERLIALRTAEVEALARRLKQTMRERAAFLTNEYPYVKKLKATFPNQHPQQQQQAQQQQTQQQQQQAQQTQDFPLTFRATRNEMYQRIRQLKLRRDEFNQQAAAIKHNLAIQRSTLHRLRKGLSIDPPETREAPPTPNIQITQDRYQCYEKAENVRLHPALHNSQNMRFSGTDNGIVTMTETVRFDMPRFRFHLQLANTFEALKDLDGKQVIDGCNIARLTFIHAETEAATRYTEANPDLLRVPKAHKMYATDIDFRTGAKKQRARLYREKHSPQGLPVVQAEAILSQNSLHDAQNLDEINARYQAQLGERNTIRHFYYTPAKARQQRQYEYCNRKQIDRLCSEERKYVKSDDGNYNRERDRHHQTIHFIGDRGTGVGSRIKGHLRYGGHWKSRKHSLNTSVCITNEHNSSQACAFCFDKVTHPVKMVKGELKTINGTAVCSNPRCLLVINKRSHQPRDSQSSLLIGLAGLCTVLSARTTFPALDPKNQDISQSNTDFQNFAGSFL

SEQ ID NO: 39 - protein, 1231 aa, synthetic construct
MSSLRTKVEQKTPFTGTETSRPPQRKAVGKGKKREHEAASTTDTSSSAQPLFKRPKSLGISSQRLMDSFFQSMRDHIIRKARPVIGSKIKIDALKTQLLAVKRLPPGIIGDAEKICQCAKNELKNHLEYHGTMQNFQYFQKYVKEHFDAIALSLQPQASTSASAGESASTPSTPVMAPADSSVALLRRDLSAVDMASSSSSASGAPSQAALSSMSHAPSDISMVDDLPEDEDYRTCTVSFKSLLRTDVPPNLVESFLQKLNAAMYDVSDYIMGMQLVVFTLMVSMKSLQLVSRGGDGGVQMKKARGFQIHDILPADYEMKIDAATSAPPPLTSSALKSATFCNGIDDLFQGKHIQYLHSSLFAERGAKTSHSTFDALSSANVTVLETISRDTPRHSSELMKVALSLYKTNLKNMWAKNKLFNKALNKLLEILLRIHLAPKRESHYQKLKEKRAKKQQAKNTFDAVAASTTGSDVDIDDMPIEELLPHYEVLDKEDEEGEQGEQAEEERGKQTGSRDLPRRRILVLKAVLRKLLVKQAGKAISHAQLKKERRDITDDEARVCLLLSEILLPYIPAKGHYSYIGFQLPFVLLANDIFRAVGYGKFAVDLCPTSSCGSLHAMRLDAPSVYTLCTKGDDPVDVFDFEGFHIPSVAEARAHKHAMFNTFFDIRAIFKACDTFGLTFANNLTILPGLSSVRILGTKKNSSKPAYSMKKPLDIDWKFVNDAQSKTDEELRKDIDTLEADMITLSEELKSQSKDHNHEELSSKIKQLNRQRGAERTLHDRDPCSEIADLKTQRYQSFLAVKRTKQQLSRLRSRAFLTRKAIEYRNKEIPAAAVTDPPPSDQELNAMVFSGTDNGLTVMTETVAMDINKYKYHLSLHNRFSPLANFDINEGDGSEGANAATSDIDKDGDQASSGDSSGVDALRGDAVTVDTSAVPQTHDDDAASDGASNHSATFQPLPTSYKMRAEDVDFGSGQFTKRQIILKAKRTTEQGKQVQQMEEQLKTSNVFRAKHVEDVMSNLAAYRNNGPALRSFYHSKKMTILSNKVEHTDVRFRSRFASRERSFCAGGRPQTHLTMFIGDRGLCVGSRLKGHMKYGGKWKPRLHSSVAKVHTTNEHKTSQTCMYCFGPTSHLVQTIKAKDGKTRTKLTNGAFLCLDPECVSVVNHRAIQGRDRTSAMAIAVSGLSSLLFNQPLPVFNPKPSQSNTGIISKTTSFCNRNETRDGSGAASSLP

SEQ ID NO: 40 - protein, 1153 aa, synthetic construct
MAKKRRYQETQFTSNPNKPPEFRRVGQRSAPVTATTTPSAKDISIFQAWKAFEDGLREVLIQRIKGARLDKKPLNKLKKELQNETRILRYTYSNADRIHKLKANTTRAKVQNSKPLDRQKKYIQENFDTIAQSSESLPTPHRTPGAPEGRQQTTATTPKQEFKAVTTTFSSIIRRQDISDERWRHFQQRLSEATEDVSNHIIDFGVQVLRLITMFKTHTFQLQGDDFQFVPSQASFTMQDILPPRYHNEETPHPPLPPPLAQQVLNNKRFQDLARGFLSEEHLSRINTTYLGIGISDNTLAKYPLDAAITARLPRPQDVPPIRFSSARAVARNLYSTNFDNLWSLKGRFTKVFNKLLTILVKIHLAPDREAKYQDYLKKQKQGQEKKTTSAQEANTGNFSFAGKKRNQVRCIFGQERKNQQRYNQKAKTMGGKWIKRARRSAECLQTYAFTRYQEVQDSQQAKSTDLTAREIRQLVATIRNVIFQHSGQAQITAKQFRDVEASVNGHQLDVCLNIINRLLPYIPATKRTWFSKMHQIPFILLANEVFRAAGYDRFAVKLANIPSTSTLAALHLDVTSIYSIFSVDEVKGRTLDLFSYTGEAITSRSTALSNADAVFHSIFDLAKINSMMDLYKLQFYHRIVLLPGFKTVRILGTKRTAATVHSARATSIAPQDLPPKPVEKKNLTVAEKKDIENKITTQQAEVNALARQMKKNLQERGEFLRQYYQDIKSYKSLFPTKTNPPTSQGDTAEERILQGTRDEYHQYINQLKQTRDDFNRTIMTTRKELAEARYQLYLRRRTTSTTPTSESVPSTTMIKQITSSNYECYEKAENVRLPENLAKAPNMCFAGTDNGIVTTTETSRFNMQRFQFHLKLANKFDVLQDLEDNPELIQVPKTFKTYAKDIDFKTGAKKQRQRLLRDKSSTKGQAVQASENICSQNSLHDAQNINDLNERYQAQLEHREAIRQFYYTQTRAKQKRKYEYIKRKQIDLLCSQERQYVNNYNSKSQTVLFIGNRGTGVGSRIKGHLRYGGHWKSKTHARYTSVCVTNEYHSSQTCPYCFQKTTHPVRLVNGKLITINGTSVCCNPNCLLVRSGRSHQPRDSLSALVIGLSGLCTVLSGTTFPALDPKRISHFNTDFQQFAGSFLTRNLPRPAV

SEQ ID NO: 41 - protein, 1067 aa, synthetic construct
MGALYATFKAKLKEAIRQCPDRMNYPELYITLFERTKLPRYTVPDAARRLDIDLRLVESFLRQQVQTTGKQTIAYWKEYVEQHFDECINEVLYVDPQETEANSSDVDDQDDDDGYDETEEFRTCTTSFQTIIKKDLEEEVRHRIEETILNCIRDTTNYTSDFSIQVLKMALVFNTHTFTLRNNTVILEEAQGANLGHVIPIEYLDDAMRVPRPFDAACFRNQAFKKDFDLLFQTGHFNIINSSLYGTKGPTDANLNKHSVHRLISDAIGLAPTEAYHHLPSHVTKMARLQYTTNVKNMWSDKTIINKLLKRLIKCLLCIHLRPTDEKQLQERRLQPRQAAPRSSNSNQGMSFSHKTRNGRRRVINNEKKNLERCKRLNKVDKAARCEERLTMYADVMERERRQKALRRQASSSLEEGVSEDEEDALVADEPESEKTNDTSKRRITQMLNQIRSNLFQNTPLSRSTVGSKIENLSDSEFDAMSIIAGFIKPYIPAQVDYAKLQYQLPFLLMANEILRYMGRSSQCLQILPLVKPTTLHTFLIDAKTLYALFCASTLQQPMILFNFQNEPIQEGMVDSYKDAVLSAFFDIAHLKRLCERHGNQFARRLHIFPALTTVRIYDTVPRLVQHQQPITATDGDPIDEDHHPDLQQQNDAVVRFQHELKDATARLETMLLMSPSVRQLKNDFRGGQASFKTVEDAKTTKRRLMLDVRDAKTNLQIARREVQRLKSMHKFPPTLPPNYQRYMIRQQPCLKKAEECFIDEDFDLQGAIFAGTDNGIVKTTETVYFDIERFLYHLRLFLHFHPSDQNGQPVGAPAMFMDLPATLTIGSKEIKQGSGMFDYMRSMNKKKTPGEQVLAIESTLSANSLFNCSTPNELEETAHLHQRHQQAIRSFYYSNIQCKERRQFEWRKERYYDRLASNERNSSGVKHPIMFIGDRGHGYGSCIKRHLRFGGHWREERHSRYTPTIITNEYNSSQTCLYCFNKLSHPTFAHNNAIKTTKGTFVCLNEDCPNAFVSVCRDQVSALAIGLAGLSQLLFGAAFPCFDEDTNPMNKTRFYEMATLFLREHA

SEQ ID NO: 42 - protein, 1149 aa, synthetic construct
MSSKRQQKRKQRESFDPNVTKINPAAAFASTKRRGSNVEITTSRKKQKITAATMDNALKSQFHDYIFDQIAASTNTTDTFSSLISLKKMPRVVIQAEDSIKADRGTISNYLRGLPQSNISLFVNYAKKNFDDIVEEVEATPSASSAGPSTAPPEPRPSTSTSTSTSTSTSTSTSTSKLIFTEDEDDGDLRGCTASTLSILRQDMLPEAQAHINHTLEQTLARVSDYIVDFSAQVRKLLLLFRDHTFELDQNSVTFPLVAPLSSNALLPTDFLPNDVQLPPHWPSKLLDSKKYEHAFGQLFTLQHFSRIHATYYGQIGTKTASPYHDAMVTALPKTDGNEAASRQLSPHAMRKAYDKFSTNFANMWSGGNRFAGLLDRLLLHLLNAHLVHRAQRERKDKDTAQTPSLQQQTDTQYTFGSKSKGTVKKIIEKEEKNVQRHLNKGNHDKAEECRRRISNYKATQIREIKEGIAGVLNITLQIYKSTSRKGKQKVPSTEIVTLDDAEEPPADESVTKTRIQDLKTLVKKLLQDDKKVSSKSLQSVFPGIKANEMEAVTKIVNTLRPYIPRKGKSGFIYKLPLILLANCLLEAIGKRSATVSISPVSSCVKLQALFIDTETIFSLFVASKGATKAERVMQITTFDDQPIKSASQALDNKSAVFSAFFDITAIHKICQSYHLSFANIIYLLPGCKTVRIMGTRSRLVHDLPPPSLPPEASSTTAAAERRAAVLKAEKEACQRELGEQKQALADFHRQTSIRKLKNEFNNTKEARQAIQTAKEKSHKLEFQVAESRRAIQMKKQEIQALTWVPPKKQPTLVDTDAPCYKRAEDSLLALDDIRNATFSGTDNGLITVTETVAFSLLDFKYHINLHNFTRKLDSAKGTLNSLQLHTAIHLLTSMAADVFKPPPPSFKVNPRNCSHWRGLSEFQQFIKRHKSKQLQQVEKRMLEHPLNVSSPVELGDNYTVHHSAAPFLRKFYYSTPIVKRRRRLEFRSKKFKQKLCHRERAFCNKRRPPIMFIGDRGFGIGSTIKKHQRVGGTWKGKLHGSSTKVFVTNEFNSSQTCLYCFHKLSHTYKKKDGKKTINRGSFICHNPECVGRFRVLPRDRVSAMAIGLASLSQVLFGVTIPEFDPHPTATKSKEFNKKAEEFCRTFIV

SEQ ID NO: 43 - protein, 1135 aa, synthetic construct
MRSRKKQKAPSINASPVNPLPTWVNSSKSAKRKADNISGDTAEASVNHTATKIEQALETEMRSRLTKKIKACLTKGDKLADLKKECLAMARIERGTVSNAEISAGFQLNTITNYLKNHSKANIDFWRDFVKSHFDTIAKEAQASIESSAERAAASSSTSAQPKPSTSASSQASASSTTSTQATISAPKAIVHQDTIRTCSANFNSIIRADLPLDIRDMLNATLSRTLEHASEYATSLGLQVLKLLLIFNNSTFNIDKNDSITLVNAQGFHINDLVPEQFHVEEDKQVAPPLPKQCADSKAFSKEAKKLLSFQHMGLIHSTYFGARGIAAQSLKANPIHNALITILPRENVQPDLENFVMKTVVQTYSTNFDNMWNNNKVFTKLFNKLLLVLLRHYLAPNREKKRRKYIEEMKEKRTKSEDGVVMRLELMADESSKVEEEVLQAEDDECKPVDDSDKSRRHINVLTSMLRNVIYQNYGNTVTEKIIKSTCKDIAQDEIDAFLKILDRIMPYVPSKQNWFSTAHKIPFVLMANQVLSSLQYSKFAVQLCPLTPVGDLNALKMDSRSLFAILCSTHSKNRRALEVYDFDGRPIPSGDAALEQKDAMFGSFFDLNKVKQICQSYGLRFHQHLVFCPGLKVVRITGAMIKHPVPCNCDGSLKKLLKADLAATDRERDQLLKDRREFRKLNQIKSYKSQWNKGDKDGNKAGSKTSGETISNNELYWLIEKEKEKRDKINMALGNNKEKLSSLRRELYLVGRYLREAKATKASKKPPAPPPELKTCIKKGDSCFLKAESTTLDESLLDQDCCYGGTDNGVVTFTETSKFDLAQFQRHLKLYNKFQCLETLGIVIEHSCLSFNRTNMPLEGAKSKHVLLPRTFKITSKHIDRGSGQQKRRLQLEKKKKSTDLGRQIATFEATKSQNPIFGATTPAEVDQLRSVHESHKQQHRDFYYSNSRCRGTQRYEYQRRRFIDRLCADERRHISNNSPVLFVGDRGYGINSRIKGHMRCGGAWKARIHGRYCPVLITNEHNTSQTCVFCFHKTSHPLKLVNKKNKQYLRCVNGTTVCTNPQCLLRAIGETHKARDSLSALAIGLAGLSTATIGQPIPQFDPNISASKTEAFKAAASDFLMRNSAGHAPRV

SEQ ID NO: 44 - protein, 457 aa, synthetic construct
MPIFAAKKRQATLKKKLVLQQGDELKKESLNIPSWAPFKLIDHGQAANDQVLIPQKPEVPGDALKDHWTPKTVRIYPTSKQKEILHLWLTATRHMYNLAVAERIRLGKEAKENGTKVKMKLKHLREVMPLKKNFDYERDPKLTYLKNVPFLIKDNGLRDFDKDYRTNLAKSKNTLRPFKIHFKKIKDKQSMTVEKKVWGLSRGIYHDVFRKGAFRSTEPLPEQLPHDSRLIREHGKWFLVIPYETSGLMPKKEAINAVAVDPGVRTFLTTYDSSQRVLEIGEDGYLKLQHLDRRLRSLQSEIHKLRSKGIVLSKEDRKRKIRNRRTVISRLYQRKKNLANDVHRKASHLLASKYNYILIPVLNFHNFKKTNRRVKANLANMKHCLFVQRLKNKASHFENTTVIEVDEEFTTKTCSLCGVRSNIGASKVFKCDNCDATFDRDHNAAKNILLKYLGEID

SEQ ID NO: 45 - protein, 455 aa, synthetic construct
MPIFPAKRQATLKNKLVLQQGNDLKKQLDIPSWTSFKMIDHGMDANDQVLIRTQKIEDPGVSKEFWKPKTIRIYPTREQKQTLQLWLTATRHMYNLAVAERIRMGKEAKAKGTKVKMKLKHLREVTPLKKNYNYDEDPKITYLKQVPFLIKDNGLRDFDKDYRTNLAKSKKSLKPFKLHFKRIRDKQSMAVEKKVWGLSRGIFYDLFNAKTLRSTEPLPEKLPHDSRLIREYRKWFLVIPFESKVQIPKPDQSVNAVAIDPGVRTFLTTYDSSQKVLEIGEDGYLKLQHLDRRLRSLQSDIHKLRSKCALPKQYRKLKIRNRRKVIRRLYQKKKNLANDVHRKASSLLASKYNYILIPVLNFHNFKKANRRVKANLANMKHCLFVQRLKNKARHYENTTVIEVDEEFTTKTCSSCGTRSNIGASKVFKCGNCESIFDRDHNAAKNILLKYLGEIA

SEQ ID NO: 46 - protein, 350 aa, Saccharomyces cerevisiae
  source 1..350: note = Saccharomyces cerevisiae Lalvin QA23
MNIKTLRQMLINKDINILEPHEQWLKEYNYDLKSEALRDLVKNYSSNMARYKSTGTPFKLKFKSEKAKTQSLSVLKKYWNNKKSFYSDVFSSTKMRSAEKLPEQLLMDSRLQRTRHNHFYLIVPMVGEEIARDEGREKFIFIDPGVRTFLTGYDSGQNVVEIGKNAVVRTEKLKHRRRKLQSKLAKLKTHKKRQNHRKALHRLDEKISRILSDMHKKSALCLCKSYETVFIPKLNFHQCTKLNRKSRSSMATMAHCSFNDRVVMKAEQLRGTSVIVVEEDYTSKTCSKCGNIKKDLGSNKIYSCVSCFSVFDRDFNAAKNIMLKYCCEHLMTSDASSVPLPLVERVRGSP

SEQ ID NO: 47 - protein, 486 aa, unidentified
  source 1..486: note = Torulaspora delbrueckii
MTTCTQYQAKKRKRSNSKTTKPYWNEDAAHISQQWGLNTILPTAAKNLSQKITSDSWFSVVKHPQVPVINDSLNLPLSSGVKEENTRARKIKVYPTNSQRETLKKWFRCQRYIYNRALKLHRDGMAMTIKGLRQTLLNRDTIILEPQEQWLNDYSYDLKDEALCDLVKNYSYNMAKYKQTGMPLKLRFKSKKTPIQTLSVLEKHWNKRTKSFWSEVFSLSNFRGTEPLPEELPGDSRVQRTRNNKFYLVVPLAGEEIARKSPAEKFIFIDPRVRTFLTGYDSKQTVVEMGKDAVVRIEKLKRRRRQFQSKLAKLRKHKKRQNHRKALHRLDEKIQHIVRDMHKKTASYLCKNYENIFLPKLNYHQCTRLTRKTRSSMATIAHCCFHDRLTMKAEQFHSTSVHEVEEDYTSKTCSSCGNLKDDLGSNKIYSCSSCSSVFDRDFNAAKNIMLKYICEHLMASGGSSITSASSGGNGGFAMMGPGPFVR

SEQ ID NO: 48 - protein, 1241 aa, synthetic construct
MNGPLPSATETRTTQYPLDRLMGKKWNNEQNLFVTHLGQYARYYHSVKRLVGQFFLIFVYDFNNMFSLSRQELEKVIPKYYDMSVLSKNKTNELYEKMENRLTLPIRWDSLIVEAIISKVMDIDTRKSSNSPRDYVDEMVAKYQILRYALLDPDLNPIGCCHSYKTAFANSICNSYSAEVAETCKRMVESALDRQDDRNIFHHESKLVSDLLEDILAKIDDGDIGEGYQALKLIFSSQPLKGMSLILTPSQVFFKYLLAFNSTFIPFSIELFVSIIVNSGIDMSLGLTDKHKILRYISKILPNLEKEIGHPLESGYPLDRFKFFDKEIEILKASHLASFPSSSDSLASTKKSFLFRYYTIGFVGKSVLCDYLQSPYVKDNPFDFNGSTKTIHPNFNDYERDNIGYIYNQKMSVLINQRAMMVTTNVPSHIWFQTKKEMEKNHSYSYNHPHYACFVAKQVGLKPNIPKVKDRDIICANCLSSHSLKDLPMVCEQCDATIPILSEHEKRLDLCGDLMDLYSVDIQLKRNAFEKMPPFDSKDDLFPPPVPLIAEINHRLVKFDQPESPHVEIFLHEALNTTSIALNSNRSGIAPSMRNLFNLIKTTSDPSMQFRIRDAYPFVHDSSTGGFENEIKHEIMVKELMNHFSMTTSKLIIVCGQFPCKAAKEAFNANPLDYEMVDYSQESYHTILDKAKGTTEDKEVDCVYKDEEKHIRAKRDFYHSGMCRRRRSKWKVMRENNRGNYLYIGIDPGRSKCTIVDSMNRARVVSVASYYNLMGTDRLLARQQNYKKNPVLSKFESGLPPYNTFDTLFNVESVLRRIEYERITHRDQCRLYSHVVTSYTFQTMDKNRNIDKKRAQYAYAKSFVPPTTIPKNRIVIYIGGSDFFRGSRGGGRTGCTSKTLLRLFLNMGYKARLVNEFNTSKIHNACASKIHSTNNNKFLEKAKHQKVKNVLPESFEKTHVVGAPISSTFSDNGVRWQGKDSKLCDGCKLTDYNCQCSKAQLADKENDSTLKVSQLADDMNETLKVFISLENTSPSPTMLLESLFSLSKKTVKELIEFCNLNSIKYNQQEEPTKGKNKRGSHKDSKGGATNSKKDQLINAIIKFNFEKQKRDASKNKKKRSKSSSTTNDRCKDQDQSNSQKDGNKSTSNSVSTHDYGFGKSLCDSQIKDLWKSMNPNATEMPFKTKWQWLYCPVCDHFFNRDTSAAKNILLLGILHQTADYRPWIYRPLPADIVHIGRVDTT

SEQ ID NO: 49 - protein, 1010 aa, synthetic construct
MIRYFALGGDAIFSAMGKHPVPRIDTVEDLSNDSIYSLCFFMKHVAPFLEKEMQKRPKMLQMLQKLRQFRCNNSKNWVNVIVINIKNNIMVSTYNHLKQLLNTYKDRKIKDLGKNQEEIKKVKKLVRDTIYNFSNTRLKRAEKDQQPVQEEGEEDEEGEEDEEGEEEEDEEKKADRVVTYDLSDGVYKTIEILGGYNQQVIDEYNNLSDDKKKKKEAKYDNRVSRRKNIIKKKKEDAHKRREAIIKERREASIKLQKSPNTSLTVDDLTNSPSSSPNTSSPRNPKGKFNQRMKHDSIRIRELDELKKITIEAEKRNADLQQISDTRTPTVFIPGLINISSIIPSLGFDIQFMFTREHAKNLLKCLGLKKANVEKDHEGNPSYAIATLFPGLYKYDPRLFTNTKRVYLPATPPTDVEKQKDEERRKTNNDNRFASALKKYKGNEDVARVAATLQTGEKAKNAIEQHNKLHGFKKGSHDKNRALLAKGTITFSKEEMFQEIVNKGVKYENTSTTTTPSTSSSSSSLPYVTVTFDKKVSVTTNGASATVTFRTIKYKPGNKPNKLYNKQVAMEKEFAGKSLKDVIAIGIDPNTCSITVVNNHDDKVLDMRMDTLMVKRNTKFLLEKISCHMGKAIKSFESCLPSLKFNVLNPQTFQSRLDYYEKYCDYFDIYFEAINDPIVLSWKRNFAFKKQKLNKEFYQQFDNYILDLFKDHFPTKYSSMNDSERNQYIQDNTIIFFGNAKFGHNTRGKVTTFAAKTVANKLKSKQYSVVYINEYNTSRQHFDCFFHQEEENLIHHDLNEPHEDPKDGFHLEKCHLKLGKEKDGGQPTNVQQCSPISEACQSAAKGNSFLRCSDHRHRLALVKQDTHSKEYKESLGFLKSIDDLDKKLQSHNLLNVAGQCRTRKVTPSDVAGSETESRQDSQKVASTQSSSSSTKKFDYRHGQNKYGPEIRKASKSLHNKHRMAVSWKRVLCTGCGKSFNRDVNAARNMLLLGIFKVWKNGHRPSRYGKTN

SEQ ID NO: 50 - protein, 888 aa, synthetic construct
MSSAAQTITASPAAVATTTTITTPPTGAVFQPFIGNRVLRSFSISKHGRGIKMLLDPKGEKIYNDGTVFMATRDYSCLNIIRKMHKEAKDGKKNHFTKTQILSSVSPFHTASQLRDFGFKFANGAFTNANKHAKQYGPGRNIRAEEYTQFCQALGRSDINKTDANKKKIMEDIKEFYAQTTITEPSSKCSTKNQKDANNKTVRVSTPIRKLLCPSIAQAYAYYVEFIAKKYKDCSDNDEKTPIHRSTFYKFVPSDVKIFNNRTDTCPYCHLYDVLKAKAEKTTLTEKEDTDFKTCKIHKEFVEKRKKDFEKKKESLTKDQVIVLMDFKENIALGMGPEELSQNFYGKPQRSILGMVIYHRDPATNEVVKEHFDVFSDVLNHCSSYVIHSIKLFFSTSELFKARHFSQIFFWSDGAKHFKSAEILSFCIDLKKSPSQSLNRVEWQFSAQYHGKSDVDRHFADLSNITAQQSKCYDIYTGKDFIKGGDRCYSFSDYNEFVFTNKSMYMGTHEVNYELDPIRFTYKFLSQKVKVGQEIDIKRGFKEVGVEEAQVVKLKQRMVNATKIKLNIQSSSSSSSTSSTEQYDEKTRISELKSLNVKQLKVINKEWTFRSKALKGEIIDTIIKYEKEAIAEKMALARAEKINQEKRDNLIYFQQQHEEKKDNKRKNTEEQSTNNNIIIQEEEEEEEEERVFKKRKTHNNAIVDDDEEEKEEEAAEANSIKQAIEEKRRKFNAMFVDQEEAIQTRHHPKKDNEQDVIMGDIGGDDDDDDMEDIPCTEFKDVCGTTPILESIQEETEERKKLKLEKKILSIVKTFEQQIREGKNNITIDKPASKPQEQGQWNLKIFSMDALFSIENFKNTIDKIIQLLANYFKGYSLDQGFNISLVPIL

SEQ ID NO: 51 - protein, 1084 aa, synthetic construct
MKDIDKCLSGPLSIGKALIDAVLCKVTQVEERSSSSLPKKYRLTENQKIRNALKDSDLAPIGTCHSYLTEFSNSIETQFLAETVETFKRHLTSLKDYIHDRRIYLLLSYEVCELLQGILDKLNGGGNDIGEAIEAIKLICDSQQLKDHNLRCIAKGKKNLEEREKEQEREKKQARGKKSKKNYDLENEMADFSKINIDGKIITIKELAETALRLDAGIIPTNDGLPQVNERMLSETSKRLKELIDSSVSPKSSWSSLDKDQLNRLKLFNLENVIWPKPLPQSGLFQYYTSGHVDKSLLVDYLVSPLPKPDPVFDKKNPPTVHINFNDYVKDKIGYIYLQNVSIRINQRVMMITKNTPGPVSFREKVETDKAKMSPNHPHFPCYIAQQVGLKPSVTKEIRKITCAHCLVSHTLNSECLPIQCPTPLCNIQIPILSEHEKRKETCGNFSNLYNVDPSLKKAASEKLVHSSDSCPPVKPAISQVDRILREYISTDSPHVEIFLHEPLNTTSIALNSDRSGIAPTMRNLANVINATSDPSMSFRIRDAFTSVRDSSAGGFEDEPKEKEMAEELKKQFESTTSKVIIICGHVACKAAKAAYEVNKKDYEIVDYSQNSYDNIYDDGVPLYENRRAKRNFYHPGMCKKRRYQWRSIRQAISSKYLFVGIDPGRSKNTIVDSLNRARVVSVATLYNLMKTSRLCAMIEKYKMPLKSFERGLPEYNTFETLFSLESVMKRIEYERVTCQHQCRLYVQVVTSYTFQTMDKNKHIDNQRARYAYAKRFVPPTTIPKNKTVICIGGSDFYRSSRGGGPTGLGPKSFYRLWKNMGYKALMVNEFNTSKIHNACAASIHLTNNEMFKDKIKHQKIENKQLPESLEKTLVVATPPISDNGVHWECNFKKDEKCGKCACKKYDCQCANGDLAKDINSSLETFITLEKGSPSHTLPLESLYSLNQKTKKELIDKIINLIIKFNFEKQKKTNSNNIQENIGHSQEDGNKFTNNSAPTHDYEFGKSLCYTQIKALWKSKYPGLDMPFQTQWKWLFCPVCNRFFNRDTSAAKNILLLGILHQTKAGYRPWIYRSLPADVLHLGR

SEQ ID NO: 52 - protein, 1020 aa, synthetic construct
MINTINHAVVMVDQLWKSELAIHQLPTKDLDDVDEIDHQISKLTPDQRKAREDIMKKYGDLHIKLEAKQKVDARVANIADEVASLIKFGDSFQEFCKRKESDRLEAERLVQEKETVDEKKKREEKEEEAAKKAKEAASRLQKKRKRNKSKKLASEKRERIRAGIEKNINDIIDFINKKPKPSAIKRSKNQSKTKASRKMIQAMNNSFIHQRISNLQKENNKAYKEQMREYKKAKEIERIISLHPSERTDPEEEEKKKKIPGMSNLSPIVPIAKLDAQKPFTSNLLEALLIYYHKDLGIKKDFNPSEALAPSLFDGMPTVTCQSVPPPSLVYSPDSMVTLDHKFASISVPTLTPPSLPPVPSLPPEPSLPPVPSPPPVPSPPSKKKVSKPSAGMKKKGGPPPVPSPPSTQKVSKTNKFMKFAGKKKLKVPAWSSFHMDTPSARRPHTLVSVSLAWNSRHPNNPIDTRKNRIYIINQLNERGVVPPPQKPIIAPADRFKDIEIGKIRVFFDKQAFFTTDGASITFTYNTIKTPNDIIDYGKKILIDEKDRVPFSPKTIYENDIINKMRDENMEDVMAFGVDPGRRGNYWTCNQLPYTFDQRGDVRSSKLGSKFLNEREKALFGDKLYHFQSDLPSLKHDVFDPQTFTDRLEYYKNPLYLEYIETKTDRLQRLNWYKNHSFRKKRLNQSYFKEAEVYMEECWKAKYPEMIQNRSAEGKKKFIKENIFGAAKFSHNSRGSPTTFKASTVAGHYKSVDYNIIMINEFNTSRQHFDCWARSEKNHLDNFNKHVPHVKGEPFQFKMGDLRKRELNMDYLDQQIMCNDKDCQSAHDNGHTHCCQLKKGSLFSLDHLQSQAGLDEFLQSNGLLDENNKGRTIDPSYRSANKKRKKKDVKNNNDTQPLNNDGMEIHGTHSQTVQCTSTSPKSDDYLKDQVLYNRDISKATIVDMPGCHVYVPWKTAYCTCCKRSFNRDKNASLNILLLGILLIWKDLYRPTRYKGITNIDEATIVICTQLSKKNKEDFIL

SEQ ID NO: 53 - protein, 962 aa, synthetic construct
MVNQIVRDHMKNQSNLNLLNIERISKHIMELNGFGPIVTSIKKYLLAKPEETVDSALREPSILIWLRQHDATITKESFDRFKSFKNDFETYLVPVTKSSETNCTSALEFIQSILNHPTIIFNWNQHTINQFQNIEKEFGKYHNEDNYKTESQAWINSIKQPTSPIPLKNNNSRWIKANDNYNKLIIINEFIPKFKLAPRNFILSTKFLQQFLQQFYNGSDFNREGSYLDHLSLLINIPEQFKQNFYTFKHPLTVCQFNLDRVSHCGHVPPVDSVDHTNAHTICMFAASNNLTLTVPKQKKVGAMLCPEHSTFSKKCKLSKCGSRCISKFKESEWRIQSIRCGSLDSEPHIEKKNQWIRDLSKPNSIIEVTSQKQTSINWTSTNASTASVKPNQEHFNSPFFVGCDINYQGMESIPPNCDIGVIFSDPTSKWHVGLTADDTDFNPSFKKLGKIINTVFKDKKVLPKLFITNSYSSTNYSPFGIPEQSLPHILAWLDQIIEGNHKVIILADWESQSFILHLFSGKPIQNSLYNISNQNSGNIESYILYHSGRPTTLFIFANHPTPGVMKPELELASRLLAKYDGDSKHFILDPAKEKKLNFKTKPSDATNTVSYDYDGPTVSLNGITFSITYKKPKTREDDESIHPSKQPYHSSLNVDLKEFQYIQDMVKEYKSNKYIFGIDCGRSSHVSIDEHGKTHSFSVHEHYLNLRSRFNSEAQKKMLGPAMLKFESLIPPSKSKSLISNRISYLRLHPTTTKNYVNIRLDEYNLIMKKNNYYNKRKSYNSMVDKLVPIKNSIVFIGSAKFSHNSRGQSSFISSATLYKWVKKKKHKALWINECYTSKVIAECHKQLSTLHQTVTGYIDGTNKVNSSTNKSNERFDYMKHHSLYSNQMSGNNVNVDGLAKDHPILKVFQEKKWKRPLCRCCGHFYHRDKNAARNILLLGLLSVRYQFRPVVFQYIKFINK

SEQ ID NO: 54 - protein, 1159 aa, synthetic construct
  VARIANT 63: note = X can be any amino acid
  VARIANT 370: note = X can be any amino acid
  VARIANT 975: note = X can be any amino acid
MYLKFNNKEKIKQSIDLIINRNIVKEVHQTNTYYHVAMLFCVLLFLKKKGKFKVSDSMVVLLXQKNSERDIRRYMKRYLEQPHMLLRCHMLFHHFRVANIQIEMDMKKQGKCFVMLAILYIIDTNIPNLDCYQYSQSRLLSSLVPFYTSKEIREFGITIDNNAWNRAIQHVHKYGAGRLINAEEISNWRSKFTHNGQMTRRQRTTMLRTAVEGYFNMKDITEESSKNMRLYSKELDADGKRAFELVPIKNLALGNINTCFTDYLKKSEVDQVKYPLMGRSAFYSFVPEYVREMKNRTDMCPMCVQYQALESKRIKFGQLSDQDHKLFEILTIHKMFVQQRKNDYLTCVIIILLIHLVNDLLMKFFFFFIXKNELKAGECIILCDFKENISLGKSKVQVSHEFYQQPARTFFGMVLFYKQGDKLKIHYYDCLSQVLNHCSTFAISCLDKLINDPLFTSLNINKITFWCDNARHFKSNEILSYMHTLSKLQVADPNTPNKTKRKYLVELNTFAPYHGKSECDQHFSMVSRAIADKSSFKDSIYTDQDVINAIKSTTDNNNYQRTYKLTSESEKEAVTIKVTVEIHNPKELKQKEIKIFPGFTEYHRFQFLDNRSVSIFFYKKKMRANKHYIKFMGGNEVDKMTIYKYSAMKSQVGKETTISRGYESTTKEEAQSWKPARDKAHTTYLNTMIEMNEIAKNVDESNKEKAKEMSDIINTYANVTSKLAPQPTPSRTQQIVSSPQSPSQPTLSNSQAASTSSQSSPKPATTTTPDQTLSGTNKRRRGYRGTKKTKQTNSQNNTQNNTQDNIQNSTQNNKQINTQNNTQNNTHNTHNNTHNNTQNTQSKPLFDIESIDFGDDDHDIGSSLIYSGKRKEREDDCDNNSQHKKINNQINNQINNNNNNNNNNSTKKRIREEDNNQESMDNQKHHKSTHVNSQTPIQSPSQTPIQTPSQTPIQTPIPTQSQTPIQTPSQSPIQXPTQTPLHTPIQHNTTVQSTEQSKPAKEIVVIFEIDNNYNSQIQDYRQQQQINYLQQLSQTLQQQSPPQSEAAASNDVEMTSHEDPIMEQVEETDDAMEIDRFCIPYEKIKDQLNTAEEDENMNNAGDFLSNWPSLRELIIRIPCLTAIADYGQLFSTLLIKNPLSNLVIRPIQNMIGNVLKSFL

SEQ ID NO: 55 - protein, 945 aa, synthetic construct
MVNPTDLIRKDIIMDSGRQFFTNTLNNLENIYKRFSNNSSSYLKIKDWEEENNNNIDTLSKYNISISEYQQHNRIKSTIEKLKKTKSTDIRITEHLSTISDLYNDSETIITQNSIPVHETLETTPDPHQYLWMVGLPLVIIHPLTEDRHLEDFFTLENYIKYLSSNVISEDYFYTIQKHVFTFHLFLKIYNILSKSKVDKAKKSLNSDAFILSLFDISKKPLDVLQSINAPDSKFFFKPSVKFQRFPDPFVAASLQPHFILIDDLLSKHLNKLTGVYSTQDRYVDRLRKIFPDLDYRNLFHNHSLKPSVPTPDISIKEAVSIETLRSIISKEELELKKPTADEINQFKKQRLCPRHLKPMEECSQTCKDEFQGYQWAVDLFSEGFFSKSKDLAKLWPKYRSTYIDDIKIQFKEPQVHDIKFSTKQPTPEPTTSNLENQDIPTQFANTPAVVSCDSNLLPIKVTDHKDIMVVMSDPISSMHLAFNREDNDLCKTFANLFKAFGIKKPREIFKLNDIKARISKRPQRKKIYSPSWLISKEGFTPVSIYEKSAPVIRTDGISLSITRGVARRFEPKLKEQHLPYQKAGTAKERRSIETLKMAVATDGTNPICYSIDPGRSSHVSYSTDSKERRASVKQYYDWKSNRYSKKVKKLEEKFKLFQSKMPPHSNAYGRADYFIKNWEVMKLYIDFRLNNIHRQRRRLEFKLNQQSKSKLVKQLVPTTASEMKQGVIFLGASSFNHASKGHRTFISSKGLKKIFEAHGYNVVSVNEFCTSRVHHGSYVEQEMKLLSAIKRVDQHVSGPKAQWPPESKPKVNSSRGSVYEKVRNDYENLSKFESDLNSGGSMQEQQSTYDYYGHHSIYSTELSKKTTKSDQGHHSTHSTKLSRKTSKSVAKREWKKVLSKDGFLYNRDSNASRNILVLGLFMALHSYRPNVFKSVRKYTDSSTN

SEQ ID NO: 56 - protein, 970 aa, synthetic construct
MSDSDFYSETDSEYVSESDSETNSESDSETNSESNPPPPTTCGTRHHSLFVGQNEEPSNRPTRYLLRQRNTNQLNLFENKFSNILKNSSISHVLRDLNEQSSIITNHQLTLFGISPHEFQNHQLIENLIKDLSTTKSAQLNTTQYLSILSDLYNSPDIIISNNILLSYKVNKSKIKTRELNLRLMLIGLPIIRLHPLTNDKHIIDYYKKKINKSINVELFYEIHKKVSTFYLFLIIYNILIESKIIKIKSKSNYNDFILSFFSIDSTPLQVLESINKANNVAYFKPKRSHQEFGHPIPGSHLSSKFVLLSEEVVKQINKHGNKYCLEDRFIDRIRKIYPGLSSNHLWNNFHHKRQDSDPNPPESSVVTIDSLLQIANKHSISLPKPTTREITSFKANRLCPHHLSSSDKCTKDCQSVYGDYQWNFNMFINGITDNFDEFKKVLPSYRSTFTDTLNMGTLPESHLSTKVNFSNRSPTLPTNTFDLDKQQHVPHQLTNVPAVHSCDSNFMPINVKDHFDILVMTSDPISQRHLAFNKTENDICQTFKSLFINFGIPMNGSPIDIDSIHARMSLRPSVKGIYRPSWMTRKIGDTSIYLADYAGPEFRSNGDTLSITRAVARPFNPAVSKDKLYLPYQMPGSKREKQHVEDFRSISDTDPTCYSVDPGRSSHVSCSSHGLVRRASVNQYYSWRSNRASKKMRRLQKRFQLFERQNPSSELDYFNLNWSIFVEYIAFKMDPRHKYLRRINYRFGQIAKSKLVKQLIPTPASHMSGGLVFLGASEFRHNSKGHRSFISTKGLKKLLEQHQYRVVMVNEYCTSRVYHGTYLREEKNLMVAINDQCEHESLPKVEPTKWPPDNIPPVPPTNQPSTVFEKVRADYAELVEFGSDLNSQGSKQEKLLNYDYYSHYSIYSTELNLKKTQVPTRDWKRVLAKDGFLFNRDVNAARNILYLGLFMSLYSFRPKVFMSVRKYTV

SEQ ID NO: 57 - protein, 1795 aa, synthetic construct
MSTYKTISDLKLEDFINNETFSSKSKQESQDATNLLFHLHNIIHSFYLKNINNLNNKKNLNNINSKDIYNQSDSITINYQTLQYIVNRLCNKKDRDETKGINKILGKGEISVLEVYKKISLENTKKQLKVFIKEYRESKVFQLLPVDQNSNYDTTQVAKQLTTNIVNHLSVQLSNHIRDIMKLYNAAIKELLPDPEIDRTETLRFLIKYNIASILPNKEINKLLADNKLENTDPTENIIRIINNNLDYSTLNLSKLYSFFNGYDFEKLQQMEKGVFTNSWKFFSIGLGSVFFKFQKNIEEIGHQVEFAQLVNSLNQHIEHSNLLQVVKDGKQLLDVLGKQEEPLLVNTSAPRDIKFTNSESHLVIWVRSIFKNISKYGDQNLDKIQKRLSFFIPAAPTSSTTTTATTPTTTTTKVSFFNCNSKNHEALGLAKPTPVVDRYRDSNICSTCQYHTNSRRKENRKDILATDIIKSLGDLKQEDNSKVNQLLVIVKDIIVEVFKSEINDNYNIDTLIEILKIEKENNQQPSLRSIFDDNDDFEFKSDDPIAATTTTDSTNDSNNNNNNNNNLEGSDQSYHPEETTANGFFDILNYFSRMPLPSGSSKDTSASRADQLQDLQMTEKPISPYSLSEGPPDPDVIFVSDHLDSMVHQKRRLNENTIYYDIKSCSWLFVDPSNSKDLSDEQLLSIKVNRIKQILQLGKGTFFLCGIALWKLLRVLITNEEVNITNRDHWYTSMKLTCSFAGRSITLFLLSDPCHSSTITKQGFQHYSNWKKDTAINIIDSEAKPTTTTTISKSPHINKSNRNRRLIFINNKMIHEDKETLGTDQCMEDDVSNDTSNESIISANDPPTPTDYYKFFNIRNRSMSFTLFPSVDLFNPFSLMYSAKDIRRIIKAEDGMTDIQVIRSILKVDFLRRIFEANPIDVHIRTNGYSGGVVFKSKSKKVQKQYELIGLKDIKDKDVLTEEQFEQLGVVVCDPAPNHNVWLNNKHPFANGDYSFRKNVIDVKSSTHHFQSESGLNKHKKNKLLGHDLMKWQSDIPSVNGNIFFSQDYQIFTNDITDLAIYEKRLEFIFKSVFLPPEQRSSDIKPYGILRAVGEEKKKEQRHKSVFHQYRQCHGDAWQKVDLDRSRSKKHSESLNSKKLFNFALQSIPIKQLTHDPMQIDSTDPNDPKQIDSTDSNTPKRVPKLVFKGNNVPIQSDRESSQEKSGANKMKLRRVGSPIKVRKVYIHLLREHLGKLDDGSLERFMDRFKFVLDITYLQDDFTNLQKEALLKFLKDHKLEFLEPIDESNVRLNDIQTAIHYYGVELIQQAMDIWKFFKFTEPLPGNQGVEKEVHFKDGINRVEQITTTFRNQAFQKFWFRRIHFERCKWLKDHGIKIMPDSVGGHWIRPNQSKVDANEDSIYQLLPYTRDVNGTFNFMVTDKEDWDSTPNKKAREICKVKNIPVVSNATYITECLRQHVYLPLLSYDLSSAANTTSSAAKVSPSKKSGSKTATNTTSSAAKVTPSKKSGSKTATNTTSGSTVAPSKKSGSKTATNTTSSDAKVTPTNKRIDSPAPSIPDLQTKKKSKKTDNTDKKSIDSDEDKDNHPKSRKKSIIGAGCGNLNHIQRMKGVKYSPATERITESLQRYSFSVSKTNMKISISNEFNTSQVGFNCLISIVFGPNIDKLENDFVSAINKPVSNEWTQAKKDEELHSLYNLAKVKPPGESKSYKPKDTKRVYNKSFSVRLYTGKDDQGQPKDIFINRDILACTNMMAIAKAYFCGKQRPQLLAPFTSQPIINTPGKECPRQNLISAFTQ

SEQ ID NO: 58 - protein, 1388 aa, synthetic construct
MSHISIGPFKVKDVLKDEFFTTISSNLSEDQRRSLVQINDFLHKFIINNQDNFTKNFPIVNNIIVKNEDVLTNLEKTTTHLTISYNIIAYIYRRIIGSEVLESNHMGEVWKLLQFQPINIHEITNIISEEDTKYILELFLSFKDEFDIKSKYFDEVPKQVATNLYNFLYSQLQSHLRNLTKVINTSIQKNLINLEINDKLKKLFYFTHYDHQNIKELGSKDFNGYKYYRLGLGGLFNKIKYTLKYLNPLKAVEADLTDALSLHIFFIKFFNVEFLLISFFIGLDDPSDVFIRLSNWGFDFPKLHPKMNPIEVTFNEASIVKIAQSILGETYMDYSNVKQFTDLVEFVERLKMQEETPHTVGEYLLCKNDNHKNVGLPDNIFKDYMRKMEIIIIFFSETVERSDLNLSSNVIDLIHQLIGLISEFLHHLNSSLPRPISSPSKAINDLKSTLKSLINQYGQDQKSIININNKPYKETTTSSTTETTTNSTTETTNEESTDNNQFYSLVDQFRNIQTSTASPISKTYTDPNYVIDTEKYPRFVGSPLNTFSMTPQVSSPTSSIITHHPLDPNDMSLNKDRKQCATANRIQTLSMFNGIRNTNWLDLHPFVANNMSEFPKEHKLIMLDSLQSILSDGPGLSLLCGSVNLQLLHTLIKDGVVVVVSKQSFQSSFSLKLKILDKSVTKTITVHLTRHPCFYSKEFISAFYYFANDIRLSIKENIDISFFYHIKRSISVLLIPQVNSFNQFSIHYNKTALGKLLNNEKINVSDIVNPKFNLTTYEMVGIRSNGDSCYIVYAKKKKKPTANKPTATSTITTTTTTTTNETTTATTNNTTTETNEPTTTKTKNEIVVTKEIKLYDKVKKDPQWKELSIIAVDPGRNHNTYLFYPGGLSDDDNGLIPNTPQQLAQMEKKFSLDMISGTHYYNSQSGRRKSIKNKKRGKQVDEWESKIPSLYVGLPSSKTAPTSKKPSTSRAGKSSKDTDVVMTDLPPVKNLPLKPLQYIYKPLDPKVLNLPEVVPENYTKPEVTITHKLRFKGSDTKRHRDLLYLTKMAIGSEITKENVESFKKRFNSLVELGMDFDFNVVKTFSEDFNRIQLQSTEIDQLRGKLAKHNLVFKVPQGLNDPPTKRSVISATQRYGTNLIQQALNILEYVKLSNSLLLKCKEELKVALMPSVELSGLCSEILVILNSKDFQTNWLHRIGYIRYCAIRRFKFDERQKTLSVSTVIPDFEFWIKNNQQSKPINLYGKGAGPNNYVQNVKGVQYSPTLLKEIDFLERRSHSRYGARVKTYLFDEYNTSQIFAPKLKEMEKSYVKEKSDAKRKEVYKAAKVKAPPDDPNSYVIKYYLDGETKYYINRDFNACLNMVTIAEAYLKGKARPTLLCPFKKDNPEDD

SEQ ID NO: 59 - protein, 1218 aa, synthetic construct
MFNHLIGRDSIDDFKFYIDSSPKDSIYLKDNYSPKKYLILDFSTIFYCLNRFLRKKEKPIDKLKFTLDNSSKYEKSYYFVLQHELNRKKSHLSEIATSTMNHFNTHIQLLLKNIMKALNIIFKQILINNNINNISNYEIFSFLIEKRLIGFFSVKQYRQIVELYNKHRKLSIPIQLGIDHDILKRICLDIDSELVVALNDLDWDFTSIQYFREYDFGQLSDLKITSKHYECRIDFLYRCCLDIVKRTTNSKMNDLTEPYFMYRFLLEIDSVINKFFFLELKNLHRLEGQIDHIGILQKLVDFNIYQTFPKMYNNGFYGVDSFFKISQMLESNQTPLEYFLLKDPTLMNSNFLSGWAGVNKDHSDGSVSDGTDSEMGTLPISQDTDAGGTVSVADDSVMDTLPISQDTDTGGTELNNAIKNIIKKSSIYKDLTNDLLFLFKQLVEFKCDAEYKIQFIILAVQELIKHLESFTTAYIPLFYDHFTNTTNLFIHQIIQLITSKFIDNHYWFIDPTNGNPVNPTGNDVLIVTHNPLSSVDRQWKPTQESSSSTIPPKVVPPKNYSNAALNLSALAWRTKTRFTLVWTELCPIVLSSINCISFVLDPILENHLIWLLETGPSKIILTGKRDQIIFGNIVKKFNIPVHLVFQTLGFFKVLIFIGGKVKKVYFIEHPYRMSQFLVHTFVELGIQEPLEYPSPIVETTTTTTTTAEVSSASAASATTTTTTTTTTTTTTTTTTTTTPASTNKRMRNLFLQRDKQWMEKEPPTNSEQECKRSYIEKPFRKKLEDQCNSYNYSLLPIFKGLEPANLLYKPDEIHQYEPDIEKKIKKIFGKKSIYLNHYMTDSLTLYVSFKVPTESAAKGKKNKLNCDISLKCPMGIDPSPFKNLYLNRPLDFEFNDKDENDEPVKHCKDLLDSVMVLNSGSHKYQIRSSFYEYKRKQVLGKDFIEYETKSPSLKCKVFNTSQTNHILTLAPYDNLIAFRYTDHYETFLEYRNQRGVQELQILNLRSLQGKKRSKNWRAAKIFTHAIREVDRRIGEETSTPNNNSSNRFIFGNGPGHQGYVSGMKGVKHAPYVARDMQALKQYIFTVTGVMVDQFVVDEYNTSQMFSPKLIENEEIYLKNSSHYEKSKVEEYIKDPSWDPHSRQSESKLRYTRKMYQVNDQPVYINRDVNGANNILLAGMRQFERPSNKNGRRFTLLMKKRGESTTAEECDDSNLVADE

SEQ ID NO: 60 - protein, 1756 aa, synthetic construct
MGISLQNHFNQWLKYIECQTHHVSQLKGILFDFIQCVNRFGSFLGRPKKFKQNNIFIRDVESIFALFLNIFLLKNHHLIGREPIDKFKSFIDGSLRDRKDIKVIFNEYSPTKFLVLNHNIVVYCIYRLLQRKEYVTEKIIQDKLYREKNRSDYFNIQRDFNLKKSKQLIKKDIEEFMKRNNINNELNNNNNLFKSTTDHMTSQFTTAIMNHFNSHIMLMLNHIMKQLNIFLKEIFNIKNNNHNNSDILRFLSEYHLIGFLSKKQYEELVKTYNRCKTDPAFQHKINQLDWTFESIQYFREYDFQQVSKFQTTSKHRELGLDHLFNRCLKVVMKNKDILDCDLTKSFYLYKFLLEIDKDFKNISNDILNDCSRILERLQRFDIYKDFPTTSHGFYGSDYFTKISKFLHEVDRPFGYFLAMDPQLEEYSYWNIRASENCNSHQPTDEIMLDAPAQSIFDNDTPLITQLKQGFEKRHQFFKSSKAETDFSILYQQLEKLDMPENQYKTQFIEEAIEEFIKHLDNFYDRSTTRNQYMERTELFIQQLNQLINLPVLKHLFINPIDGQPINSNTTPDVLIVTLSPYEVDHEPQTTKPRASKGSATVFLSKEQLMKQDFISFLKEELERGNLDIDQLLARGVLGPSIIPPIQPSEDYLCHGGEAYCVNLLPMLSPLIEPNCPAEERLKSETALNLSALARYMNGHCSFSWTELWPMMVKSIYDIGYVFDPLLERHLLWLLQYQSPPTIILSGKRDLIILSNLIKNHKLPVHVVEQQLWNFKLLVSIGGKVKRIFATNHASSLSPSLTKFFHEAVDGLYPDSMYLSDSDLEIVPYQVVISDKVSDIVAVSDQLPVSGQLIASVSDQISGSNQLSELSNQLSVSDSMIIPDQLSVVSDNESDDTLLSYSDLESSDDSSDSDLVPVQYREYPVYRVRVPDQVANDQAPTVDKPETEKEFLGRKRNLFLQKEKPWIPRKPRSPNVPKIPRHYWKSFEKKLLDYSASYHYSLLPIFKGYELKLLLYKPKDVDQWLNLQKLNKLCGKKLSEVNHYMTDAVTFNYSFKSESTDNTKSSSKLTVNCPVKLKCPVAVDPSPFKNLYLNKQLEYNFDGEKHCKDLKDSVMVLKSKSHRYHTGSSFTNYKRKRNLGPKLLEFENNVPSLKSSKENDILSIQTYEIYIDFRYSHENYSTFAEYKLVRGGPEFTEQHLRSLQRRKIAMNWRNRRIFNHAIMAVDRKIGEEKAKKTGDINDIININNNKNNNISTSKGSSSSIGSSSINSNNRFSILEYEDNNNNDDDDDDDDNDDKKKMKLRGKSYYKYKQQEINYYNFQLALYKLYKLYPNEFLPYPPPQSLPNRKRTHTNKINDQLFPIVEYGRSSGNTDIHELLVDIEEWFRDKFQKANGWEKILGPFLTTKSSKLFSTPSTTTTSTTTTSSATTIPTTITPPTTTSSTTTPTTPTDKSKCANRRHDSVYKDQPLGTVPVFFKSGKVTKTCKLCLDFIDKERKEKKKKEEEEERRKIDEDEEEEEEEEEKQKDRKKGRKWKCKNCGIYNNYYVDEAFQDRDRVWLAHCNNCNEIKSVFYDKEVAERYALRQIFIYGNGPGHLGYVKGMKGVTSAPYISKETESLQKYIFSVAGVKVDLLIVDEYNTSKMFAPKLIENEQIYLMNKSHYEKSKVQNYIKHTSWSPNSQQTESKLRYTRLMYILEDGEAVYINRDVNGAGNILLVGMRQIQDSNNESRFTLLMKKKSESTTGKESVDNYLFADK

SEQ ID NO: 61 - protein, 603 aa, synthetic construct
MRLLCCTKTNSLTLPWSCWSTCLSATTSGLWWLARAIQLWPPLSTPSIHSASWPMTSLPSPPSLWRNKMASTLQRIEEHTTIEQPWRKAEEAQQASLLRKKARNKAAARKANKMRQMAAAAAAATTVVGGEKGKGQVGGGSRGKWKWKGPPSAEGGGDGEGGMVAFNGKMRKICVFPMAQQKTLLKCWIGTVRWTYNQCVAVVCRWQCAPTKKALRAHFVTEFGLREVEQIKRAKSGVGSGGNNNNVGISWVFKTPHDLCNQAVGQFVTAYKNAVQAHGWGKFDIAFWLAKRLQQEMVVIRSRDWNRTRGEYAPIFGNTMLRSSESLPAKMDHEFQVTRTKLGWYYISIPVPLDVKHPIQQQQPDAVGGGDNQAPAVAPHAAAAIDPGVRTPFMVYSPDEARVYELGANDFGRIRRLCHHLDDLVSRMTDRDVRKKRRRRMRRAAARMRRRIRDLVDDLHWRAAKWLCETFETIIYPHYETSNMVVGKSKRRGLHSKTVRAMLTWSHFWFKQHLLHKIREYPSGCRVVLVDESYTSKTCGGCGRINHGLGKSKLFWCEQCGFRTDRDWNGARNIWLKFLTEWCNGSSRGNDDDDKEQQQQQQQ

SEQ ID NO: 62 - protein, 653 aa, synthetic construct
MSRGNGRGGCRASPVSIDSYWARCRFLANNTPPPPQPNKGTDWRALESSSWSTSLQRTMSGSWWSVRALQPTPRPTPCSKLSLTRRRSSPTTSSPLPPFSWRDITDCALRRIDERALLPEQQPWRKQQAANLKTAQKKANRKKASAAKTKVMEASGDGDTNQPGGKRKRKGKERAPISEEGEEEEEPAPYDGKCRKIRVFPNRWQKDLLKSWMGTVRWTFNACNAAVRAGLSGHSEAELRSRFVDNEAFGKPSRRKKKKAKVAAPFSERGKELLAATRHLPGPSTLWVLDTPRDIRDQAAKELSAAYKNGTKAHGKGKFEVKFKSPKKMAQQCITSNARDWGRGRTSVFHGLFDSGRALRASEPLPREMKHEFKIVRTRLGRYYLCVPMDLETRGESQAPSSSDDVGAECVFIDPGVRTFVTTFDLSGRIHEFGTGSIGRIEKLCRRLDDLISRTYAKKPDDRQRFLRGKKKRWRMRRAALRMRRRIRDLIDEAHRKIALWLCENHRVILWPLSGVSNMVVAKEDLKQRKRRIGAKSVRAMLTWSWYRFQEWLKHKVREFPWCRLVLTSEAHTTKTCTHCGTPNHDVGRSEVFRCADAACPNRSAHRDHHGARNNGLRFLTEWANLPTTTTTTTMTQRLPTRVIDLTTSGLND

SEQ ID NO: 63 - protein, 768 aa, synthetic construct
MLDLYKQGDTFWKDLLKNHKPLLCSEDTSYVSVKLSVLSGSVRCTTTVTNMMPNRTKESVKRVTNYQPGARARITVTRDKTTKEEKVTLKVTGYEAKVTEKEVSDFNQPDGLDQVIQYAGIGFKTVVLNNVWYPLFARLGRLTKAYMHQMCKKHHHKKTPWSKAHAVVQLIRSGKPIPAWPAYVVAYIKNARAKLLDAAGSTMYGSLNGHALQARQDELLSAHENKYLWDDHAKKKMTFQAAMQFNFWMQKQFESLHQKKMRLMPVFAVKRSHVRLDTKSLLHIFCSLFPNDHNVKLMKSFWNQDAFLEKCGLPGSAHPDTYMLPPKPPDGTTDKALLAQYQSACSRIRNTDAYRSQQARHRALSETEDRVVKSFFRCLPVKNGYKFDCSISTDGVAVSLQYSKPSKAVTAKRKRSAGMRSSNVATDAYDQNLDMYDPEEGVLTLGLDPGRCSLATVSVVTQYTLDSTGKLKLGEKLNRHSWSLLRGEYQHKSGIRTSTIKKAQRDAPLQPAFQALAVPGCALRTAQSGTVLRYIADSQHCMNEWWERALSRQEAMASFQGYIGKRKALDSFFSRVLKEAKKLVPKRTKIEVAYGESGITMSPAGKGEVAVPTTGTFKACQRCFGKSAVVPTNEFRSTMMRWESGSKTHAVYKEFRAAEGQDCVKEVLHGTDQKHGSACQHPTQGFRSSWPGKQGGRARTSNAEGPSLELQRRSSSQQLAATLHSQWFRVTRMYYNRDTHAASTIGRLRIMERKGLGRPAPFCREAKA

SEQ ID NO: 64 - protein, 413 aa, synthetic construct
MVLAEPFICLSAFKVGRITGRWAAFTRTMDTDGTAACFHFRVRRAAVGPAVARAVAARGVAARSTQRCSEHCWREHPDVEVVGVDPGCTNIIYTVKRVAALLQPPLRLQRPLRLQRHKVYKLTRKQYYMESGVVKANTQSAAWQSLPGHAAGAGDTGCGGRSKCVDAEGFGAVRRCGRGAPEAAVGGQRLRLYGSKECVFARFFQRLQDGTQAGSKKKVIVAYSAVKFALVGPGELAVPTSRASKECCQRLVTFPVDEFRMVAVWNGDKATVLQDFWSQSLMWQKQAPYKDGPSMLCRISSTRLTNLINKEALSQLLGQVTEAAEHSLHIRLHRVVIGILFVNEVLQEHAGDRLLVALLHAHHFWVRDGRADVLQRLQALLKARVLAHELLEHRLVDTLVATIGQVIECHVCC

SEQ ID NO: 65 - protein, 830 aa, synthetic construct
MAPKRRRDEAEKAEEEKDHTTSTKCGLAGLLSEKIEADGVAVTREESLAAVDFLVAALTRLRFEALCLLGLVAVRMCEDARREGQGLQPHCATCRRLRKTELVEDDMYAAICAVSVCDLTEQGRKRGRPSKRDQHPEDDLFRHVCEEHFPRDEEAAGARVNRSGLTPFLPPLSKGVFTNVKNHYAANFAAWLARSFRCRIDDELRELRTPATKKLDKLAWSMAHAVLYDGELEQPRWWVGWAQGAAGAAAAAAAQGAGPAGGAAAAQAWTALVDYVNAQRASKRAAELLLREVKGAQATYKKASTRHMEWAAEILAGLEARRDQLGAQVQQLTQAQPLTREDTQRLASLRRELHRARPFTLTPSPSFAPIYVPLDNTSMARLPGLLPTLARRHGEVFAGAGAGAVAPSSFVQAAFGGGGMQSSATLNAVGWGLFQLGGVTSRNAPFANYITTDGVACSVAREAHNKPLANLKPATAPADAEELCTLEEMKATQIIGVDPCGGGNWFMAARSPLYQPGPWAWEGVGPAQRYLLELHDKQLDEELFPGQLPPEPRRRRKGVHRRKQSKHWQPRARTARRRRQKRGRFHMSMGHWRHMSGLERLQPNRPQLAPALQAYVGGIPTAATASAARFEERLRYLFASGAAGQAAGGPAEAGPRGAVHVLWHYHFSAFRRKRWAAFIQRDRALHRVAKQLTGGRPKEEVVVGWGSWAFQGGKGGSPISVRGGRAPTGRLIKLLRERYAKHVFIIDEYKTSKTCYNCGCQEMAIKRLGGLKEGQRPWSVKVCNDCLTTWNRDVSAANVIRVLLLLKLMGFERPTKLQRPPWPPAAAGPG

SEQ ID NO: 66 - protein, 906 aa, synthetic construct
  VARIANT 34: note = X can be any amino acid
MVQKNSTRKVSLRSVCKDEATLAAIRFKVDFVAXPLRYRLLDFLRHFVVCRTDPAFHCAVCDTRWREQCGLAARRQGLRERADVSPQQHVFHRRVPTIDINLLQESAALITTSTHRRQRQSLLSHFYDAHFAPLLSEDTGGPAIDITAHHQLVHHLLQDVLTNLEQHYRANFPKWVRKYCRAKLEVNDDEVPLGGLAGMALGQPQAGNEGGAETDEEDEEDQVHVTAKARKALAGLMTAALMGWRQLPLTEADLGGLAVPDALLARANATVAPLVAAWTARGIWPIQRGSRGLLQREPLCLVSAAADLLCGLQPIRRWLATVYERLKTANPPAPPGEVRSANFAMKRAQTYALIIEASSMPLHIPLTNTIIQELLVNHKQLAAAEYAAAGAELLGSNATAVWQAATAAAKHAGGWKAFANVQQAAGAPAHHDAWRDCGFNFDWLKRLERGGGTFAYFMRTDGAAACVTLTDAAGSSSYLDGPGRAPGNNPVPGVVAAGQALAQYTPDALKAMRVVAVDPGCVNYLVACVDYLADFSPHSHQHPAHAVRLWAGPRDIPEAERQQLAQGPTRPLRERRRERAKRRRRQRRLRGVGRANVWRKRQRGQSRGRGRRGCARGRWRRKPRGWRGRRSRKQRHAATKRRREQRKCYARVSGAGYRKRTGQSERQRWQEARLQEQPALQRWQAGIPSARVAYAADHTRRIQYLYGGSGGIGLWQLLRHYRQWGQRRWRLTVYIRSQKVLEQTAQQLAGGRPKEEVIVGWGNANTGHGGCVSRSGRGPNRALLRLLVDKYAHLVVYVDEYYTSQVCAKCGRRLLGNGQRCLEVVIPFGGSRARDVQVCQHCGTVWGRDANSATNMRHALMEMLLGKPRPAALRPAGAHVRSRGGGQGHVEEDSAAPPSKRRRRAG

SEQ ID NO: 67 - protein, 967 aa, synthetic construct
MTNRISTRKVSLRSVCDNDDTLAAIRFVVDHVATPLRVRLLDFLRHFLVAMCDPHWHCEVCRQRWRRESGLAPEGHQLRDRSGVQMLPRKQRHNRGHPDISLDMLQEACALVSTSTRPQQRESVMAHFYAMHFAPLLVEAACGPPVDITAHHQLAHHLLQDVLTNLEQHYCANFMRWVRKWSRAVLEVPDEQLPPAPAADDDDEEEEDDDDEEEEADAGARVFKALASLMTAALMGWRELPLTEGDMGGLAVPAGLLDRANVKATALVQTWKARGLHPVQAGQQRLLDRDPLLLVSAAADLLRDILPRHRWLVSEYRRLRTAAPPPPRGELVKAKFAARRTKPFSLVVEASLMPLHVPITNTVLQEVLINREVLVVAWEAAAAAERDNGGAAEVHKAVQSAVESGWRAFPNSAASFEPWLQRQGLEPGHDPWLVAGFQTRRNAWDGWEFAGFLRTDGAAASERAGVAAAAHSSPSRGRAGSRARGRGGTRAHNSPNEDGDQRGPRGGPGRAPGDNPVPGVVAAGDPLAVFEQATLSPMRIVGVDPGVGNYLVACVGSMPGFSPHSYSGRPRAAHVWRGPDGQAPGPAQPPLQRRRRRQQRRWQRQQRRQRRQQRRRRRGQKRRWRRKGGGRWKGKRRGWRGTRQHTHQRAATERRRQQRACFTRLSGAGYRHCTGQTGRRLWQQARLEEQPALKRCQAGIPSARVASAAEHELRIRYLYSGGGVDAAAAAGSAPAPVQQYGLWHLLRFYRQWGQRRWRLTVHVRTQKVLEHTAQQLAGGRPKEEVIVGWGNANTGYGGCISRLGRGPNRALLRLLVDKYAHLVVYVDEFYTSQVCAKCGRRLLGDGQRCLQAVVPWGGSRAHQVQVCNHCGTVWGRDVNSATNIRHALVEMLLGHKRPASLQTGGGGGGGGGAAAAAAAAAAAAAAAAGARARILVAEGAGKATWRRRAQRRPRSGASAQAEEPAGV

SEQ ID NO: 68 - protein, 944 aa, synthetic construct
MTSPNIHFQFLLIPSSDTHGRPAIGEKFHFEQARAAQERFDWGRGPRLPPRRVLEACGEAPLSRDCSQGTIKTTFKSIADPPGLEQLLHAQHPTAAAADVQRLASQHRDSAVLLQKALQVLVWVMTAVRQEAFDLLLLVVALCAEDTRLQAAGQPPNPERFRGQSSIAEGFRRQRQRACGSDQPHHVTNDAAQRALRGFPDLNQRVMEDCITAVTTSEHHQEVQHSDQRAGRDPHSHNLAQYVYGHVFQVHRSEDRVGPPRSVTGLTSVVHQLARQMETNVGVHLATNLFRWLKTRCRVQLHRTLHPEAAAAAPDAGAGGGAGVELPRQQQTIVLALAAAIARDEPLCAYPDAAAALQSQPDVLQELELWVQQEQDRWRPRGLLVPGCSFKRHSLPHGERYLAWAAEVLHEIEQHRSQLRARGVPKAKVARHFPVFGLLPEPSYQQVHIGLGQEAVKELLVHRDHFASEHHLIPPAAVPTGKFRNIPACFKEWVADHGLDPGDVWAAAGLRLGISQHEHRGSKFDGFVTTDAFDMCLTMRRRYHSSWDEAKEAGKQVRVSEEELLRLSLRGVDPGCSPIYWTCAMRWPWLPAWVVALLGVDLVLRLPEVRNGSCPVRWPDDLTKDQQAAAEAQLAAHLTASKAAAARDTIVTRTQAADRATAAASTAAARWAKEVQAAPQQGRPELVPPRPLALMVAQSAEHSKTHCSGAEHRTNCGYAQHQRWQQWRVKADPALCAWQSSLPTARTVDVQNVLGRLDYKYGGSTGAGGQQGEAVVRELGFVDLLARYAAPAWRQKRLQVYCQKQRVLCREAVRLMGGEPEVTQQNGVVGWGGGSAGFHGTVSRADQPPRKGFVRLLRERFARHVIMINEYRTSMVCANCGRLSLRRKEPPQGGADFRVLVCNDCHTTWDRDENAALNMRLLLVLQLLGRDRPAVFCRPEGGVD

SEQ ID NO: 69 - protein, 676 aa, synthetic construct
LPVSVLPDLLAICHSFSRKSVAANPAHISGMEWRHAPILRGLRRLLDEVAAQLAQNLDQHLNGNLFRWLCRRCRIKPASEDLVQLVRDERDKAWSGRGRGRQHLERGTDFAIWAADVLADLAAALPQRQAAVEAALQQVQQLPLPQQQHGVAAQQLRAARERVKDAELYALSPQPSFMPVHIPLTTSILRDDILPRAADLARLAARIPQAQLPQGAHIHPQHRWHSRASVAAGGVVGQPGHAAGGGGGGGADPWAWLGISTGVKKLQTGSWRFANYATTDGVDFCVGMHRPERPSDDDVKEEEKGLRLTVQQLLELCLFGVDPGGLLQSGAITILERDMDGDLVEFVRVEGPADRLQRQLQQYRADRKQQKQRKRRQWRQHGSRWKHAPGGRRRPNGRQQAATRARRKQRSRFYSCSGAGHRQQCGYNDRRRWQEWQLNANPALATWQSNIPSARVLTVQEHDLRLRWLFTGPGAAPPGPGPGGGGGGGGGGGGGLPLPPPLPQGPRLGVVDILVHYFNPQQRKKRLEVYIRKQRVMHREARNLTGGRPKQQVLVGWGNGRAGWNSPISRKGQPPRKEFVRLLRDHYAAHVVIVNEFRTSRVCFECGHMALQARLKPGMRRAHGVQVCNHCGTVWNRDVNSACNMLLLLVLQLLHGDDGRPEVFGGRPHADADADG

SEQ ID NO: 70 - protein, 678 aa, synthetic construct
AAQAQAAAETSAKLQRFTIVTGLLSVLQNRALRHPINEAVRDTTFVKYETLTLLGLYVAHYCKEVRRRAAEGLEALPAMPYLGATFISHCIRLVTHSDEAPRWNQLPPDELLLSVYHHLYLPSRSNNLRRVNRDRRSEQLHYLAIQLAVNVQLHVRLNFQRFLRRWCSMRLYQDFGEHQPSPRGKVLDRMVKSICAEQCLNTDLPEGLSAAQRAWVQALVTSQRAAHTTLLPADARELRRQGQKWQQRQRQQKQKQREEREAAVKQQAEELRNNPGIPAPPPRAQELRRKRKKKKKKRQQQARVEAAAAPTAWRQPRVAPFSLMPVAAFTPCHITLNSDGLRVGAANALKGDTSRVLSLQAMVNTLGKKTKAAGGRALGEEHKQQMLLRDVELWDAHFDMWPLCTVNHKFALFLTTDGTQASVHMKRPARKPPAPPSFPMSLVGKQLLGLDPGRRHFYTAVSDSDSKEHRDVRRCSTGHYYERAGFTARQRWMERRLAAAGLQGWQSRIPRHATADLAAQQQRVAYVTERLDELVMHYCSAPWRKKKWSVRVQKQRVLANEVRRLTGGRPKEQVVVAFGDGTGGPGGCIKGSRMPPIKQFRQLLQRFATVLVIDEFLTSQRCPRCEAQYTSRVSYAVPVCNHCGTHWDRDVAAAKNMLRIARAEAAGLPRPADLRRGG

SEQ ID NO: 71 - protein, 734 aa, synthetic construct
  VARIANT 214: note = X can be any amino acid
MEKEVEDPGGPVSPAAGKKRKTVPSPEEQEKTKKQKREARRQRLQERRQYDEMVVKACLLGHIKDPYKEKLREAIRNRVESYSMSIVKASSGLMHLVREMYRDVTDIETVEVPEEFFDKTFIRHLMLGTGETSRRNERVHALHENYAEYRFNGTRYRGDSDIYAYGAMKYLTNLKNHLTMNLERFMIRAVFALYPGLSRKGIWAIINGITNDRXHEQEIEFVDKKTSRRSTNEASVIRAAIQEHRAVLGLANPAEKVSELKKDKERCYRLILRYFVFLDRELERKAEMELSEEKNEESERRKAALMGKRFNVVPLCNIKSHFVTIDSRVLHGIMKEISPEFDVSREEFTGENRETYWKNIFDFKRLKASKQKVFTGMIETDGVAMCVHYRRLKADRPVPPSASPVAKHEEKKEADPATQEVQENDFVVGADPGNTNIITIAAPKRAEDGTDGNLRQKDMRLLRFSRARYYRESGIMNARKKIETWNAGMKEHLEAMSEVTSRGADFQAFREFMEVRVAHWEALWEEYTKPRWARLRMNLYCGKQRAFANFFNQLSALKEDESQRLVVAYGAGRWTTQKGTTPAPTTRTYKECARRFVTIPVDEFRTSYTHHELGCTLQRVEMEKCQRSPEDIEKYGALTEEQMERRAKVRGLLALVSTTSDGKKRMEFVNRDFNAAINIRRCAVLEKRPPELTRENFVGQPLKVELYEKKLEAVVGGRSKKTGRRLHVSWRRFV

SEQ ID NO: 72 - protein, 802 aa, synthetic construct
MGRKRKKDDYPEEWDVVDETQDPDAIPPLFEIVAETQDSDAEVDVVTNALSQLPPPSQKSLAPPTYRIKETTKEINSSVICKLTRICKFPLLVGEIKKVCVAMKQVQLEGWHLANLHVLRCLKEGDEVPKIEQMFFYRCCTATLGNIEKRDRGSTESKYPAFHKTCLRYWAGRERESTYEPEFIQTAGDMVNEMAKLMEINALNMIALHFRQRLHQYIQFRYAKKGDTKLKYRETRRLVDSCYRVKSEPEKDDEGNPTGKTAKVWTEWDDTDDPVEQELREWLGIAPWQWQIRANPAHFLHKLYDMLVWMEKFVDKHPKTKGARVYSLLPVATTFQAAYVKLNASTLHGLLARLIDLPEVEDFLKKELNIVPTRKRTGSQLPFDKKTFQKNRSEVIRKVFDVEQFETLDRKFADEIKTNGYGASITMIRPVTTVSVALVEKKASKKKRMKNDGTAELVSKEKTARKKKVKTPEEIAEEDTFAKELFKLGPDYSPNVLIGIDPGMRSLATAVSVGRLQRRRRRQKSRRGLHRRHRTRRARRKERVIEISTPKYRHLARMNDFRFYNENLKMREPWYAGVIRAMPSFKTSSYDLYFQRLQFFWKHLRFLLVFSAEQAFLRWRFTQDRAKMKALDTLAKRIVPKANKQVCIAYGDWSRRDGIKGHASGPVKGFVEALKRRATVIPMDEYRTSITCSCCHQRLKQARLFTKMKRKEDEVDIRQKERPSKKEVKEIVEMAKFKNPKLADKKVVLKCTRNVLRCTNSKCKANFWNRDVNAARNMLELLKSGLKEKHGARRLRAFRRGQ

SEQ ID NO: 73 - protein, 643 aa, synthetic construct
MTGYKEGEFFGGFSSASSAPNNPLSNPPRAMKSAGKRKRRRKPEKQKRRRPDVIARRVANADTAVSVVKTTLNSFCKEEGKALSWESVLRDMNKAVLEAYVLANIHVVRLRQASLPIEPLNQNFFHRCMTAVSSGIRESQDCEQLKQSVALYNEWRASDVSRANIEYVARGWQQNAAKMMATNATNAINLNFYRRFHKFLKRKYGFNGRQAYATLKSILDPKYKEEDPIVIEWRARIPRKHDGKLDCKPHVLLPLTPLTLTYMFLSDIEERNQVNKTTPDYVEIRSFSLLPYKRGFECSHFKVCKTGLRALLKRARVDVPYEGAGWNEVADNWWRVFFHIDKFETANRKFDGEIVTDGKSVSIVMRKPKRESVTSELNAKDYDVVWGLDPGRTDLFVATNQYGEKIKCSSREFYDHARYKKRNQKIKIWQENRPAVLEVIRNMPTKKTAQLERLKEYVKFMIPRMDLLLKFAMVKPFRKLKLRSYIFAQKKLRELCLKLTAQGGRRTIVGFGDWSNQDIGGLIKKCPAGPVKPLERMLRKYCRVESIDEFRTSKLHNDCHRSLDHQYSKRKCRDGKERTLKVHGVLHCKNNGCHGMTVNRDVNASKNMLLLLQLKMRGEDRPPAFCRASTQEEEQQVSTSAVS

SEQ ID NO: 74 - protein, 799 aa, synthetic construct
MSNKRKKKKEDPDNPDSWDIVPDSQELEDVYSTDFDIDDGGEESLSQDLFAMTVTEAPKRKKTRTKKVTAKSKKTQTRTGKPQRKKSPGTIKEKEFKKGKAYISVVCRLETICLDSRLCDEIRRSAHAMKQIQLEAWHLVNLHTLRCLENGLPLPDYSNKTFFDHCCAGVSTTNKTHHIAQKNPDLWESIKIYQSQRERAGLSEVTHLIGYGDLKYELREQMVVNAGVMIREHFRTRLRLYVDITFGGLGDTLTKKQKKEKADLVKAIMHACYSVDETDVVEALQMRDILTPDGVGWSEQWIPWPDRIKENGMAFYVRLLWEFQDVVEKRMEAVPNEKGVQAFSLFPVSTNYSRAHIKINGTTLAGFYSRIQQRVPEYRWDLPSVSLSVRSFKENRWTVMRRAFDIAQFETRLSECPLSKAEFAQLSVEEKYEHASHLFANQVTTDGYSASVLMYRPMTEEEKADKESEKKYEEKADKGPVVPPGYVPNVMIGLDPGMRAICTAVREDFRPESQGDRPPRRRGRRQPRRSHRRRRSTRKKKSDRPHWIKRPKAPRHRNGRDIVSVTTREYRHLAGFNRCRVWNEGQKKRHPEYQAIITKMPSFKTASYETYLERLEYFWRHVRFLMQFCVDRPFLKWKFFRKRMARVAVDEIARRIVPTVSRQTCVAYGNWSRHKGIKGHAPSPVKGLKEALRKRATVVSMDEFRTSKLCSQCHQPLSSVSYSVNTMLPKRKKRKGMVLARNRAEIEFEEKECYAVLRCDHKDCEARYWDRDVNAAINMLELLKSEVLGLGRMEAFQRP

SEQ ID NO: 75 - protein, 809 aa, synthetic construct
MKSAKRKRPPEDSDSDFEEGVTPRIASLRLRVAPTDEIVQESDRGSSCDESAPAVREVIILDSSDEDDLLEDIQTMTVRPKSVKRATTRKNASAKKPKKVSAKTSQQQVRVGKEPAARKKSSGTVLENAFKKGKQFCSITCRLETVCPDPDLGQQIRDCVRRMKQIQMESWHLVNLHTLRCLEEGIQLPLYDETFFDQCCSGVSSTSRSHNTANKNPALWETIKIYRAERSRTNLKEVENQVGLPKMKYELRQQMVANAGVMLREHFRRRLCAYVKVVYPNAKEKDVLHGCYNSEPTKLEEVQEMRALLTPDGVSWCEEWIPWPNRITDDSKIEFYIRLLWKFQRVIEDHIEANPGRSTKGVRAFSLFPVATSYTASHIKINATTLAGLCSRVKKKDPETGGDCSITKWWGVDHIKIDHNAFAAEKWTVMRKAFDIDAFETLRPDTDVSQADFRHMTVEEKYAHASHLFANQVTTDGYGASVLLFRTKNELDEAEKQQSERLVPEGYRPEVVIGLDPGMRSVCTAVRERFVPGQIKRVRRRSVRKKRRRKRSWIRRYDGPKRKNGRDIVEVHAKEYRHLAGFRKRRTWEEVLKNSNPEYGTAIRGMPSFKTSRYKVYLERLHYFWKHAGFLLEICWRKPFLKWKFFNKRSSRIAVDALAARIVPDPSVQTCIAYGNWSRRDGIKGHASSPVKSLQQALAKRAKVVILDEFKTSKLCSTCHRELSPVSYLVNPDLDKRRKRAGKIISRYHSEERKLVECHAVLRCDQKKCEASYWNRDVNAAINIVNLLKYEIEHGGRMAAFARVQDRRD

SEQ ID NO: 76 - protein, 617 aa, synthetic construct
MKARQSNKRKRPPDEDEIVPDSQEYEESALEEVVSDLSHLSTAPVNAEIKPKRLRPDVTARRAMNTGTTFSVVKTTLNSFCKEKAKALPWESVIKDMNKAVLEAYVLANIHVVRLCQANLPIEPLNNIFFYRCLSATASGVQESRDCEQLKQSVALYNEWRGTGVDRANKKYITDGWQQNASMQMATNTTNAVNLNFYRRFHKHFKRKYDFDGRQAYATLKHILDLKYEGDDPIVLEWRARIPRREVDGKLNSQSHLLLPLTYTFLSDIEERNRVNQTSPDYVEIRSFSLLPLKRGFECSHFKMCKIGLRGLLKRAGINIPSKGPKWNAVSDEWWRELFNIDKFETANRKFAGEIVTDGKAVSILMRKPKQKIGTRELKEEDYDVMWGLNPGRRDLFVATNQNGEKISCSSREFYEDARYKKRNQKIKVWQENRPDILEAARNMPTKKTAQLERLKEYVVFMIPRMDLLLQFSIVKPFRKLKLRSFIFAQKKLRELCLKLTAQAGRRTLVGFGDWSNQDIGGLIKKCPSGPVKPLERMLRQYCRVESVDEFRSSKLHERCHTPLSHQYSKRMCRDGIERILKVHGVLHCKNNGCHGMTVNRDNNASKNMLLLLQLQI

SEQ ID NO: 77 - protein, 642 aa, synthetic construct
MSSHAMQAQVSIALGGLIPQLAAMSRKLKRKRSDEQGETDASEEAKVAKSQRPDVTARHAANANTEVTVVKTSLNSFCKDKGKALPWEGVLLDLSKAVLEAYALANIHVLRLCKAQVTLQPLDQGFFYRRTSAVAVGHSGVVRTSRDCETFQRSVAMYNEWRAAHVERACNFYICAGWQQNASREMATNASNAVHLNFYRRFHKFLKIRYDLDGKQAYLMLNDIVESEYEGEDPRVLEWRRHIPRKHNSKVDNQPHLLLPLMYTFLAYIEEHNRLNQRTPGFVEVRTFSLLPFKKGFVGSHFKMCKTGLRALLQRAGQKVPGAGSEWNAVANATWRELFNIEKFETASRKFAGEILTDGKSVSVVLRKPKRKSTPCNINPADYDVVWGLDPGRRDLFVATNQLGDKVSCSRREYYEYAHINESNQTIRHWQHSRKDVLEAIRNMPTKKTASLERLKEYVDFMIPRLGLLLTFSVVKPFRKLKLRRFISMQKKLRKMCLQLTEGAGRRTIVGFGDWSNRDVAGLIKKCPSGPVKRFERKLREFCTVVPIDEYRTSKVHADCHTPLVYQYCQRLCRDGVERRLKTYSVLHCPHNGCFGMTVNRDANASRNILQLLQHQMQGTPRPQVLCRGGTTKSTSVHGCPV

SEQ ID NO: 78 - protein, 618 aa, synthetic construct
MLHWYLSRPVEEQVALPKLDQSFVTACFTPGLEVRPGRAWNEGYRRVIEEVFQDKFTNYPFIHRSPGDYQSITVVAQLYITNITASTWTPFVGRQLDYVRFWLREHGLESPDVSAKRVQCLINGWITPRTDNQAGAGGGGTGGGGGGGALKVTATARAGASATAASTAATAAAARAAARVAAARAREEARARASAGGRCGKGGGGGGRPVGQIPDEVQDFINEERRLLRLEGSTRLDVNEKTPVEGLLLPYLYHILYQYHQMHPAVLAVQEGMGDKPEMQAEHGGNPGYRAFTIAPICSIKRHFMTLDTRALGDLLKNVMRRRPTAMTKEAWSEACPDWLRRLGGMGPKRFCRDAVSVKEMWDETFQVGNLRNQRVFHNRVSTDGVSVSVFFHVAKERLKREKAERVRVEKEDEEKEMLERQQGQENLQPVRERRIVAIDPGRVNLVTALDSQTGKIKTLTRRAYYEKGRINECKRKFSSWEDSLDDINTAFSRTTMKSCCPDLRSAHRELIVRHYHRLWDARCEKKRARAHMAVYAGKQKVLDGFFAGLTGPGQAKPIIAYGAGIFSHTGRGEVSVPVKRVLRVCEKHYRTVYVPEHLTTKVHLDYGQRLNPIARRA

SEQ ID NO: 79 - protein, 660 aa, synthetic construct
  VARIANT 260: note = X can be any amino acid
  VARIANT 413: note = X can be any amino acid
  VARIANT 561: note = X can be any amino acid
MIVPATVLAAAVGKRKRGTGSSRNQQRQAKRVARRERNQAAAKKRKTSSGAVTTKRTPKTTPRKRKTPVKPKPTWTFTTVKMKLRTFCKDEGVALAWDSVLRDMNKMVAEAYVLAAVHVVRCCEQGFDIPILARSFYQQCLSAVSGGNLHDRRTTMTEEFRDSVAIYSAWQGESLIPNRDAINRGWAQVAAERMAHETQNHLQLNFYRRFKQYITLRYGLAGRDRYLVLKGILDPEYDGDNEIVLEYRELIPRNDEGASXERASELLPLVHMFLTFVKDHNREHEGEPGFEMARTYSLLPTKRGFVCSHFTMSSRGLRAMLVRSGYDVPEEGSDWNAVADSMWRRLFNTNLVETSKPDGSPSRFFAGEITTDGKSVSITVKKPVRGRQELQMSPETAEFFSTRWGLDPGRRDXLFVATNNHGETIRCSSKEFFHAATYTQSNKTIRHWYDYADEEVSEANRLVPTKKTCSLERLEAYVRYVVPRFDMLLAFTMRKKFRDLRFRRYCQSRKKLHELCTRFVAGRDPARTIVGFGDWSCSDFGGVIKKSPGGPTKRFMRELSXYCRVELESEYRTSKVHHGCRQAHPGDLVTQYQRQECRDGVVRNRKIHSVLHCLRNNGGCGITVNRDVNASRNILRAFELRLSGQPRPPELSRSSQQRST

SEQ ID NO: 80 - protein, 726 aa, synthetic construct
MERRSCYSRSSLTTSKSRRMPSKKKRARPPGSEVPNRSKARRKASKKRAPPPDAHDTGGPGPPKRRRPDVAKRKADAKTVDIVARKVTLKSFCTPEARALHWNDVIMTMNHAAAEGWLLANFVVMHTIENPTGGGVLPPMDDKFFRRCMATVSKKSRPKKTFKAVFQKSKERKVQPDLALEEWKWVDFERAADEYLKHRGGARPLDCKYICLGWFEQAATQMAKNTETSTWKNLARRLRQYVRVKYPEHRADAFLITSWVLDEACNRRHCKSCRTKKRQKKPVPAECQSCDAVNGIVGALREDIPRNQNQKVDWKSTDVLRLFHKYLKAVEEFNETNKDNPKIRQERVYSIIPTKQGFRSSYFSMTSTGLWDLLRRSRDRLDLDTIVYTEGGGSISLGDSIKKENWSGVADIWWRKLFRVADLEKGTRLVFHGQICTDGIGASVLLHKPKREPKAEPPLRKIENYDEIKGADPGFTDMFVACTYNVRSQTFSDDFLSYPAKKFYSDSGYSSSNRKIRRKLGHNAEIKKVFKDMPTKKTASLTKLMEYLDYTVPKMERLFSFFMTKTFRDMRFRRQMLAEKNLRAICRELAGEPGTRTLIAFGNWELNNSSGIIKKKTPGPVMKLRRMLTDYRGVDVMIVDEYLTSKMHHGCYSLAPMENQQVKHRCRDKVVRTVGVHKVYHCLKRNGGCGATVERDVNASKNMVEVALTQMREGEGVRPERLQRPS

SEQ ID NO: 81 - protein, 716 aa, synthetic construct
  VARIANT 204: note = X can be any amino acid
MGDTAFGGVWYRSRLAQVGLIPQFARYRVSARSPPPPADSTPVYTYQSVKMKFNQLCDLDFLTAEIRTMASELKLVGLLGWKIAGKHVLRCLQEGASLPALDSNFFYQDETEKRTRNSPEYPDLHPTFVRYWELCERLPEYLPPSSVNSGPMRRAMADTMETNTINMLGLHFRRRLHQYIRFRYATTGKLQLGYKATRKLVAGXYRVKEVPHLDVDGVDTGNVVYVWDEPRSTAERELHDWLGFVPWEWRIRQNPTHFFVKLYDMLQWMEAFVEKHPKTKGARLYSLPPYSSSFIPAHVTLNITSLHQVFCRLFTDPERQAEVKTYVKDELGVECGSTEQVRRQFSENKDAVFRKMFRLNGLETSTREFAYQITTDGYAASVLLRRQTLCREDDDNADQPRKRQKNNKGEVVKAPEAVEEDALEAAMKKLPPGYAPDVMIGIDPGVRTVCTAACEGSLPTRQSRRRRNRQGKRKHKIRRAKHKRKNRHRQRTVAINTREYYHMSKAGKHRFWNNGLREKEASYAKILDGMPTFKTARYQVYLDELRYFVKHAKYLVLFSARHFFRKWRFLLYCMKMKAIDTLAKRLVPTPSPQVCVAFGGWSCQDGLRGNPRDPVKAFRKALERRATVLPMDEFRNSKLCSCCHQSLRRARLLTKVKDKDTKEDKIVLKGNRNVLHCKNSRCRAHFWNRDVNAAINILELLKCRLLDLARLAAFQR

SEQ ID NO: 82 - protein, 785 aa, synthetic construct
MEAGWHFQISLQSSPPRTSELAPSAMERAACRPPGRLRDWKRRYACSTVRCKLGRVCPHPELINEIQICARLILKSTTEAWALANFHVLRQCEERGDIPDLTSSTFSPGAVESSAGAFAEDDVRLGDLYDSAVAFLGARPSTYTYEPDDVTKIDVLHGEAGGLMSVNATNMVVTQFRKHFTLFVRLWYGISAKETRKVVRACYSSDASERREVLEMKAWLGSAAWGEDGELEQVFPRKTVIKKHVGYFVRKLYEIMVFVEQNQIEQRRQRAQSMFSLLPLSQSFALYNIKIDPSTLAHLCSRIYKRTGENVLGVELTPNNRIPTRPYFTQRGSELMRRAFNITQFETRRTKIGKARFNGLSVDEKARHAARFFGDSIRSDGYAASVLMVQLTRLARSVGRREENEEDEELGDADEFDDDPDSESLVTETNGEEITEDEPELEPEEDTTHEQDPSIASEVSDSAPYPAPEPALRGIRARGFVGVDPGKKAPCTAAIIGKLCSKKKKPPPPPPPSPKPPRWHRNKRCPVEDRVSSTGPPRTPVRRRREPKLPKVQHEQRILQIKAGEYQHMSGSKKFNKWHEKLRLRFPEYQIAISSLPCLKTADARVFLVRMEQVWLSLDYFLDFGIAHNFRKWRFFSHVKHRIAVDDVAKWLIPEAGAHVCVGLGNWSQSDNIKGGPKPPLRPLEKAMRKRATVVKVHEFRTSKLCSACYQPMKMAVFLDPDDPGPDELDADERPLYYDRSVLRCANKNCKKNFLNRDVNAAVNMQTLLMARLQGLPRPAEFVRQ

SEQ ID NO: 83 - protein, 324 aa, synthetic construct
MTAVLASRTQFFRMKNEGKKTTFPVLGFRSERARSNSIGIRTKTEGFELVDDGGKHYVQFHSKFFGASLGKGIRLSHAVPTPEHMARLIRVRGGKYYFAFSTHKEFEQSAGTRVGGMDPGVRKYLKRRYEVIDARKSRLAKLDNAAKAAAKDRESTKLHTDQKVANAKTVKPPKSREHRRRARLRREIQQTNTKITNAVIDFHHKFASWIAATFKTFLLPSFQTSLMVRKYEVEMAADGTPEKRRICSPTARRMLGLRHYSFSQILKHKMERAGGRLISCDCGKIKENLGGKEVYKCDFCGAVMDLDLNGAKNIIAHEPASYAE

SEQ ID NO: 84 - protein, 788 aa, synthetic construct
MKRKRREDNDCPDGSDEYYPSLEFVAETPDLDYEEEKSEDVDVEAVTEAPAQMFPCSQRPMISSTYHLKEITKNIHSSVVCKLNRVCRHPWLVEEIKKVCVDMKQVQLEGWHLANLHVLRCLKEGDDVPELEQMFFYRCCAATLSDVEKRDRPKKKTGYETFHKTCQLYWSGREREPSYEPEFINYASTMINEMAKLMQINALNMVALHFRRRLHQYIRFKYAGEGKLQLKYNETKRLVDSCFRVKSVPEKDDSGNLTGNTIKDWTSWDDTKNPIEKELRPWLEVVPWQWQIRANSAHFIRKLSDMLAYMEKFVNKHPDTKGARVYSLLPVSTSYQAAYVKLNASTLYGILSRRILDPKVKGFLDTELNHIPFDRNTFQKNRNEVLRLIFDVEQFETRNRTFAFEVNTNGYGASVTMIRPVTTSSVVVVEKQVATKKRKKNDETADTKKKPTKKKLKAPKELAEADTFAKELFKLGPEYSPKVLIGIDPGMRSLVTAVSTGHLPRRRRQKSRRGKHRHHQNRRGKRKERVVQISTREYRHLARFNDFAFYNENLKRREPWYASVIRAMPSFKTASYDVYFGHLQFLWKHLRFLLAFSAEQSFLRWRFTRDRAKMKAMDTLAKRIVPKADKHVCIAYGDWSRRDGLKGHATGPVKGFVKALKRRATVIPMDEYRTSITCSCCHKRLRQSRLFNNLNRKKDEADVRLKEHPSLKEAKEIAEMAKFQNPKLANKKIVLKCTRNVLRCTNSRCKANFWNRDVNAARNMLELLRSGLKGKHGARRLRAFRRGL

SEQ ID NO: 85 - protein, 611 aa, synthetic construct
MDEKPRVKRPDRAQRIAENKTTDYVSVKTTLNSFCKRGGRFLPGRALPWEEVLADMNKGVLEAYLLANVHVLRLLKEGKEIPVLDHNFFRSCLSAVMKLIVNHKIKGELGESLKVYNASRCVRSLQANGGYINQGWCHNAADQMATMTKNALSMNFYRRFHKFLKRTYMIDGRKAYTLLQGILSHEDYVPQGTRFDSIVQEWREKIPRKGNGRLANDAHRLIPLTYTFLKDIEERNLYGDQVLDEGEQYHELRVFSILPTKRGFECSHMKMCQLGLWGLLKRAGYTIPNRGSGWEEVRDQYWRMLFNIKKFETVNRKFAGEIVTDGKAVSIIMRKPKREAEPTKKYSEDDFDVFWGLDPGRRDMFVATNQWDETVSCSSKEFYEEARYTKAKQKISGWRDRNPQVLEAIRNMPTKKTASLEKLKVYIAFMTQRMDFLLGFSQLKPFRRLRLRSFIFMKKKMRKLCEQLVPRGNRVVVGFGDWSNQDVGGIIKKCPAGPVKVFERELARHCTVIPIAEYRTSKVHFDCQHELKNQYSQRLCRDGEIRTRKVYSVLHCLNNGCRGMTVNRDVNASRNMLRLLQCKLRDEDRPVAYTRQWTKKNSAIECLRVAQ

SEQ ID NO: 86 - protein, 483 aa, synthetic construct
MRADSSTEETEATKATRKKTQETSGGFRSRSRSRVIETVEPLLTSWFEIDNLAQATPTSSWRTGSRSVAHRYHEIDEVLQVQGIEDEESVNSAIKIQLHSTKEQKKKLDQMFAAQRAIYNKMVARSLPRFMKATKSSIAGFFAALKRDEKTSFPVLKFKSKFASTNSIKIPSRAVKVVEQDSHTAFPFHTRFFGFKEADGIAVFEGIPTLTTSVRLHRLREGKYYLCVPRLRSFQKTESVRVCAIDPGVVNFATVYDPDGRTFSVTDAHKVLKKKFEAVDAMKSILALKDNACKDRHKLKVRSKKKTGRKSPKSKEHRLRYRLRRRMRFTYRKATRCVNDLHQKLSCWLSENYNIVLLPSFQTQEMVMRHLREVASNATPETASTEDNAAINTRKMRSPTARALMAQAHFKFKQLLKYKMKRTGGRVVDCEEEYTSKTCSSCGVIKTNLGGDRVFQCANCHAVFDRDVSAAKNILHKNFSLLT

SEQ ID NO: 87 - protein, 335 aa, synthetic construct
QRLGYPLQCLRNPKACRDLVQMILRCAKNHNSTPTLVLLCYVVHLRSVVAIAGTNNVFSSGNVFPTFNSPATQPTPQAQQQGQHRRHWKRKRKRENRHGQRIVEIEYYHSAKLDKQRFWCRNLRKRETRYRELLAAIPSFKTADYDVYVDRLQCFLQSSGFLLSFTAQHSFRKWRFLTYGMKMKALRVLAKRLVPLRSAQVCVAFGDWSCQDGLRGNLHGPVKAFRKALERRATAVVMDEFRTSKPCSGCHERLHQARLLTKVKDEDTKEVKFVLKGNRNVLHCKNSHCKAHFWNRDVNAARNILELLKRKLLGLGRLAAFKRWRLGRKHDWDGD

SEQ ID NO: 88 - protein, 369 aa, synthetic construct
MQAVKVSRDQFFKLIKQKKRTTFPVLKFRSVNGSPNSIYLHTHNTYLKLVAEGEDHYVVFHPRFFRAALGKGIRISHAVQSPGHMAVSPGSAETGTTSHSPRTWSSRRAAPRREFGTVISTDGTTFSATDQGCSKRRFDAIDALKSRLSKLDNEAKAEVRKRHPAKIHTAQNLATNKVTMEASIKIAKSVKQRERARLRREIHAINTKITNATTDFHQKFTSVLAVKFKTLLLPSFQTSEMIRRYEEEMEADGTTLAPAQHYIDRRGRKRRIRNSTARAALSQRYFSFRMLLECKMRRAGGRLITCEEEYISKTCSSCGKIKANLGGSEVYKCGGHGPPSLSRTSRCSFDEVEPQRCCLHRGYLPLWTL

SEQ ID NO: 89 - protein, 328 aa, synthetic construct
VSGKYKGLASVYFVAAKKIRPINVSINNTVLNNIHKGIGFVEIVDEDLWQYYFNTKLVTRGQSHSVEQFMVIDGMSASLIVSRPDKATVEAIKYKERDIILQMHETAQRIVAIDPRRSPVFTAVVKLQMNHSRNTILTISSMMLYNGLVGSSIMNAATHTGTNMIRKVWCNSSAVITSFNAAKPSNTTGSLKLYKVYARYILHDLDDGLKFYSATSFKRLRWKTYIQLYMEMQTSVRQEEAIGNEPVPTRKMRQKIGSRAKTIDFDEYGTSISSCCCHTQMEGGSHKIKQMVSDFMVCGVVLYTSCSRKVWDRNTSAAINVLKIFLRK

SEQ ID NO: 90 - protein, 330 aa, synthetic construct
HGLIARIRRTRSTKREREATIECSQATQGHQDGFAECGWRGIQFSLCFPQLDSISERSCYHRKPDTKKDFLYYDLQCQTNDNYPAILRMTEFIEQAGSRPRRFSPFRTSVISKHICIDTTIVVHLLYDKELRMGKILEVLTKGGIKRNESLTESSSDGQDFRGPDKGWNQTQRVTDRIFVGIDPNMSALLFCTSEDGRNISETLKISDGRRYIARVRKDHTEGKKTDSNRADGLTIEQWETSLCGHNHQTVKCDEFKAYIQAELLFNSQIETFYLSQVDLSRFWNTRKSEQWMLQRFKEMLGEPNEVDIRIGDWKQKKHRKFRACQSKGC

SEQ ID NO: 91 - protein, 890 aa, synthetic construct
MESEENDSSKKPISGIKRKRAKSGSSDKAPGDDDENSHIHAKKELATWAVARGIKNPQTRVCSVTSSMDACVREDVPGKDVLLRQIDKDAKSLGILRHLVSVCLNVLAEKDPSNTLIVNRTFIQQLFTRISGNTLHKSTRPLHHPDLQSYLDESKLDDETLRQVEKFPLRCRDALCGEMMTSIKTHISGNFESRLVDHLSCRLERRLWECRDDEHFWASIPRAAKNIYEAAGQKTPEQALSIIRKFVDQKRGSEKELLRLEWGEAMEGLLSEFRGLFEPLRVKRPPPKDPPDHPKTEEELTGREDFLYNVKARLNDVLQIEAGCRKVDFHLRKRRGEIWSEVNERQQSIKSSKTKEGFLKQRHLLQEIPVPPWFDEGIEMSKKDASNLVRDVRNTRRDMWKSMKEPGTEHPPKAFSLMPHFSLTRAFVRYDSQSIGTLAKSLDFPGVSENQVTLKTTGVKCAGRAERLWWTGIFDFYSERIVNRRPSTEKNQTQRTRKVLRLNNRYRKGIGVCNKDPWFTDEVLYSAKRDDPSCETPWIVNSISTDGLQVKVCLATLAKTNPLPKGVKELVKKGYASIQDKLRVSKSSKGVFKEAFPLYDTEKSRLSSPDNHIEVVGLDPGQVSVYATVRANITSPETFKASDFKGSQSSISSREYKHQSLTRFSARAEVGRRLDNVEYGQAVQAFDSVSLKQPDSSVAYSSVIYCTLRVRVVELLSEERRLQKFARLRARQRTVDGMARDIAFGNTYRQEVRKEQRSSAEPLSPERRKELLKKIRDKRRVVFFGNGQFGHGARGACPRKALIRALGVLCPVVLVDEFRTSKCCCSCGVPLKQVDGSRVFRCSSQMDEGRSCPVRYIDRDANGSANIGVCGVFQLLGFKRPSFLCRKSSE

SEQ ID NO: 92 - protein, 693 aa, synthetic construct
MEVDTENPDPRAKKRCTRKSGVEQSLHAEGDEESSSTRQSCICLDLERTLVKSVMPKYIRDRLERMSRYTGSFRYIVGLFSNYVVIRRLEQGLQPPEVKKVFYDRCWAAINTRISGKNTRNDFSDLLDEFIDQSGFDTSLFPPRVPCRLQETVTREMEVSAKNSIVVHLEAKIKGFLRFRLMNDSQLAFQHLPAKDRSRIIVSLSNDCLEREMRGDLDPGYVPHVLQVRDLLAQVYSTEPDVSVLKVLKKKPHLFLELISMISHVAEEASDNNRDLREETKTRSDEEKVRKAFYMSERIKRGLVDRPKMCTVLPVWKLAPCFPYYSSSVVGSIFHKEGHDFSSVSRFISEHFDLGRVNRKGYKPSGFRSDGYQVQVTFMALVSKKPHVPGTKDLAKSGYQIPKKVVSLETQERGLFVLSQARKDSRKIVADRLHAYNLTVVDPGCASVVSVRSCPLEFCRCVGSVRERSTAWEMKGTEYSAKCGRTMLEGREKKRRSNDEYGRCFPRFSAVKKKTARKSSFLAYCRVAAETFKVMFAEKMKRARKRSRFHSSRLVQKTVDKLASNIAACPSDRKSIVLFGNGSFRAKKGHASAPRKKLVRAICSRVNVGMLDEFRTSKMCPGGCGGEMTDVQGGQRVRQCTTVSVGVENPCPLFENGVAFRCDRDASATLNFCLAGYCGLVRKSWPTHLLRRQ

SEQ ID NO: 93 - protein, 757 aa, synthetic construct
MVRPSSDGNSHKKKKHKKIPPRAPPKNIPGFLQTVASLYPNQYLTASGNLPKSCKGTYCCQLFSRVRFGKEVSQQLRTRTVSVGFRAATTDEKRDTFQSKVNDAVDHLSRERVLASLFANFVFMERLQNGSALPEPDRAFFKSCLSSCILSRGGGSLNADFDRFSEITGLTRLVPPKSMNLDQQREHEAQRMATATSTRATHHAEKRRISITRWFLRGRISRGDLDCKKYATKLYKLAEKIITNFDGTPEDTQKLRIMSRAIDPGTDTNIIIDFAWQEKEFSDRDRGDGKSAETIKHLNWMFNTYVAESRRMYEEAVSRSHLFAPGKDKACRKKRDEFVKHELGEEFTYAPPKDTAVLPICSTGAVFIYIDMKTLGSWGLLHSNDAWWYKDVLRPFSKEANVKCLVNKENARYASSEHGYLSSLLESGPDKCPWMIGESFLTDGVQIKLLLKTLEHGRKAFSGSSELNEEGYNKLPRADVQLESLLGEGRGVYNISSLLKSTSIREDVVVMSADPGQAKVINVSSATARTWAKQDPGVILKTSTCVFGDDYRRDTLASRSDEYETFRRKGQPYGVAIDRLGNEKKRTSCLATFIQYCRRWCVNGPVLFRETLQTGRKFMRFDRFRATQKTLALIADKYMGHVEDTCRVMLFGKASFRAQKGRASAPRKALIREMASRGVVLMVNEYNTSKMCPGCGEDMVEDKERRIRSCKNFKIGSPEESCRLHRLTPEFEMDRDDVGSTNIGMRGFGKLLGQVWF

SEQ ID NO: 94 - protein, 779 aa, synthetic construct
MSASLLTETSLEEEQRREVNNNTSKENDTMEKKTETAATTEKDPEPSVSKRSRNKEPKTTSTVYTSVKCYLSSIIKSESSRSNVTSTKERFEERCKSVSKMMVKGSLFLRLVVDECLRRYNHLEDEIDKWPDMTKDNFYVQLLRKGLDKKKLKEGSTHPVVEDVWNSPIVQETFLSQQGEGNNPIKRHLMDFNTITYAAKQLKTCFETNLRTHFRTRQQRAISGWLAENGFDKKYTKLVQHWIIGCTYKSDWVDSGDLERVKEGTKNFVTLHRKHLCVISDKKNGTISYSPEEKYPIPSILNYYKFLQTEYPQNKKIQKMIVVPKHKLKIHYCTFDQTTIQGICKDLGVWKDMEERHKQSEDILYKQGWYLLFDVKKIKKLRPNWNFHSIQTDGEGVSVLFSREVEEVETVSKKSKKNKKPRGDEDRRNYPPTNAKYVVGVDPGRTNVVSCSVFDTRQKRVVRKHRMTAKQYYQESWMTDRRKANETYKKNNKEYKEALEEITRYDNGEEIINDGNGDTSTPTKKFEAYLKVVNEHYRLLWNEKGKKKYRKNAMKVYSRKQKCISNFIDELIPKRDKIEDYHIAFGDAKFACTGRGEQYASPARIFAKKIKERVGGDKRFTFVDEKYTSKVCHRCNQPLNMLEKDCFSPNKKRKPPTIVTTTTTTTTEEDEENGKWKKATPLRENRDTRRCSSEKTQFGYSSNRKVSTGDISMETPVPSSTSSSFCTPTSITCVLGGKFVDRDFNASTNIVHKFLGFWDKKLMEKKDKMPLKYHFIRVA

SEQ ID NO: 95 - protein, 572 aa, synthetic construct
MPVCKSCGGLDHQRRSSTKCANFLPQVRKRKLEPQQVNLDLQVYVVKRGLQSALCPLLSDDDRNRLLYEIRRDVRDLSRVYIELGILFNYYMKRCDTLPDKPDVLNFFYAMKGKGPYAACYKHLAGDQVYDGKLRSFVVQELAKQYETLIHTNIETHAYSRLARYFGVKKNDHGLFLAYYKKDFSQATEDMIRVCQIINVDSCKKNWWSTVPLWLKIQQDLFDRDNVESFRLFPQPSYGLKHLTYTSRGWHELLRRVKPDSVPSHWPNIQDHIKELWAPWLKSTKNFGCCLQTDGVSVSLSMNRWIKKVTKNGITKEVVIKKWKPSYDKLIAVDPGSRVPVAAFNSHGFYKRVTSKFVKSHSLDWKRERVRTKLTKSSETAEAKDRERVQTTCNIVLSSKNTSHVVEYTVFRLKWFDVRQKPYETHSKLTRLMFDKYINTAKLQEKIVKEVFDGGGVEKTLVLYGAGSKFMNAAQFSGRKFKHDALLKKLRQKRNIHVEIVDESYTSQACSTCNQQTGEFSKIVMDHKKRVGVCPNCASRFDRDQNAAKNILINFMRSTVCRPRCSYTKRVS

SEQ ID NO: 96 - protein, 592 aa, unidentified
  source 1..592: note = Helicoverpa armigera multiple nucleopolyhedrovirus
MPQSCKSCGGEGHSRRTSKSCANYLPPIRQRINWDGISTEEFDVGVVKRGLNGVLCPLLDEEVRRRLTAEIRSDVIELSRLYVLLGVFVNHCWNAHPADESNSVRTFQDMMNYVYALKGKGPHAQKFDEMVRYHNGITYYNGRLRNYAVQEMAKTYWTVLKTNITTHAYSRLARYFRVKRNDPTLFAAYYHKEFGDDDISIVCRFINVSSDWHSTIPMWIEIQREMYARGEQSFVIFPQPSHGLKHVTYTSKGWHELLRRVTPLTITSNWPSITDHKKELWAPYLDTNVVDMKKFGCCIQTDGVAVSLSMNRPKRKPTSTLPKSYVTNTFNEDRIVAVDPGSRVPVAACDSRTGFKRITKRWVRSHTLEWKRERYRSQKLRHVELNEVADRHRVELDRGVQITCRNGHQVKLYTDFRLKWFDARQRPFERTRKLTRLSFDKYMMTARTNERIVRETFLNDTPSDKVLVLYGAGVNFVNVACYSGRKFKHTDLLRRLRSRRNIRVRLVDESYTSKACSDCNELRNGTYTRLRMNHRLRRGVCPDCNADVERDYNAAKNILVNFQRSAVCRPSPSCDSGVTTDKDSHSIRPIIQ

SEQ ID NO: 97 - protein, 571 aa, synthetic construct
MPLCKSCGGLDHQRRTSTKCANYLPQVRKRKLEPHQENLELQIYVVKRGLQSALCPLLSDEDRNKLLYEIRRDVRDLSRVYIELGIVMNYYMKRCDVLPDKPDVLNFFYAMKGKGPYAACYKHLAGDRVYDGKLRSFVVQELAKQYETLIHTNIETHAYSRLAKYFGVKKNNDGLFQAYYKKDFSHATEEMKQICQVINAESCKKDWWNTIPLWLKIQQELFDRDKVESFRLFPQPSYGLKHLTYTSRGWHELLRRVKPDTTPSSWMSIDDHIKELWEPWLKSTKNFGCCLQTDGISVSLSMNRWVKKVTKNGETKEVVIKKWKPSYDKLIAVDPGSRIPVAAYNSNGVYQRITSKYVKSHSLEWKRDRVRNKLTKSSEMAEAKDRERVQAACSIVLSSKNTSHVVEYTVFRLKWFNVRQQPYEIHSKLTRLKFDKYINTGKVQEKIVKQVFDGGGVEKTLVLYGAGSNFMNTAQFSGRKFKHNALLKKLRQKRNIHVEIVDESYTSQACSACNHKSGVFSKISMDHKRRRGVCPNCSSSFDRDQNAAKNILINYLRSAVCRPRCSIRRVS

SEQ ID NO: 98 - protein, 606 aa, synthetic construct
MPLSCKSCGGEGHTRRSSKSCANYLPPIRQRRNSGDVDHYTQDFDVGVVKQGLDGTICPLLREDVRQRLKVEIRSDVVELSKLYILLGVFVNHCWNAYPDDESNSVRTFQDMMNYVYAIRGKGPHAKKFEDMVRYHGNIRRYDGRLRSFIVQEVAKTYWTVLKTNITTHAYSRLARYLGVKRNDPTLFAAYYHKEFGDDDSISRVCRFINVSSDWHSTIPMWIEIQRDMYARGEQSFVIFPQPSHGLKHVTYTSKGWHELLRRVDPSNVKSNWPSITDHKKELWAPYLDTCVVDMKKFGCCIQTDGVAVSISMNRPKRTSVRRKAALPKSYTIDAFFEDRIVAVDPGSRVPVAACDSRTGFKRITKRWVRSHTLEWKRDRYRSRKLRRVELNEAADRQRVELNRGVQITCRNGEQVKLYTDFRLKWFDARQLPFERTRKLTRLSFNKYMMTARMNERIVRETFLSDTPSDRVLVLYGVGVNFVNVACYSGRKFKHTDLLRRLRAKRNIRVQLVDESYTSKACSECNELRNGTYTRLRMNHRLRRGVCPDCNADVERDYNAAKNILVNYQRSAVCRPSPSCDSGVATDTDPHSIRSIFQCAVSEPGS

SEQ ID NO: 99 - protein, 608 aa, synthetic construct
MPPSCKSCGGEGHSRRSSKSCLNYLPSIRQRSNCDDVSTEDFDVGVVKRGLNGVLCPLLDEDVRRRLTAEIRSDVVELSRLYVLLGVFVNHCWNAYPNNESNGVRTFQDMMNYVYALKGKGPHAHKFDDMVRYHDGITYYNGRLRNYSVQEMAKTYWTVLKTNITTHAYSRLARYFGVKRNDPTLFAAYYHKEFGDDDISRVCRFINVSSDWHSTIPMWIEIQREMYARGEQSFVIFPQPSHGLKHVTYTSRGWHELLRRVTPSNITSNWPSITDHKKELWAPYLDTSVVDMKKFGCCIQTDGVAVSLSMNRPKRKPALRKKPISTLPKSYVTDTFNEDRIVAVDPGSRVPVAACDSHTGFKRITKRWVRSHTLEWKRERYRSRKLRRVELDEAEDRHRVELDMGVQITCRNGHRVKLYTDFRLKWFDARQRPFERTRKLTRLSFDKYVMTARTNERIVRETFLGDTPSDRVLVLYGAGVNFVNVACYSGRKFKHTDLLRRLRSRRNIRVRLVDESYTSKACSDCNELRNGTYTRLRMNHRLRRGVCPDCNADVERDYNAAKNILVNFQRSAVCRPSPSCDSGVTTERDSHSMTIRIIFQCAVSELGS

SEQ ID NO: 100 - protein, 358 aa, synthetic construct
VIFPQPSHGLKHVTYTSKGWHELLRRVTPLTITSSWPSITDHKKELWAPYLDTSVVDMKKFGCCIQTDGVAVSLSMNRPKRKQTSTLPKSYVTNTFNEDRIVAVDPRSRVPVAACDSRTGFKRITKRWVRSHTLEWKRERYRSQKLRHVELNEVADRHRVELDRGVQITCRNGHQVKLYTDFRLKWFDARQRPFERTRKLTRLSFNKYMMTARTNERIVRETFLNDTPSDKVLVLYGAGVNFVNVACYSGRKFKHTDLLRRLRSRRNIRVRLVDESYTSKACSDCNELRNGTYTRLRMNHRLRRGVCPDCNADVERDYNAAKNILVNFQRSAVCRPSPSCDSGVTTDKDSHSIRPIIQ

SEQ ID NO: 101 - protein, 625 aa, synthetic construct
MKKKKKGEFQEFRNNEKSAYKTFKIPIKSILYNYDTTQPVINNLVFEINDLMIHAYQFIRLYVLSCYTNKQTLPIIDDIFILYCIKTLGTRDNRGKKCKDTALLDTLEKFYLEEYQPLLNHEKTNLKNTTFLLPYLATQIHTSLSNNAQEHFIQHFLRFINTTTTAITEDKSILFKLKHQLMSLDNETNEMFNEWKITHLHNIFPQNIKKSIHYDVKVRPFEYLKGMLYMNEVLEKEEHKLFQPLPLRTNIIPKHIILDTASLVSLFCPETDKDGNKNKKGNLLSNIKDNQYDIWNSFLNLNHKIFRNQHYQFHHQIQTDGISCCLLFIRKDLKDKKWGSRVPSIPEQDFYNIEDLSKEQLDTLKDRNIVGCDPGKRSLVYMMDKNGNKLQYTAPQRKRESKAKTNQRILLEEKKRHKIIEKETELSLQNSKSVDYIKFKTYLVEKHKLNKETTEFYKKETWRKMNFRQYSYGKKSIDTFLNKIQETFGSNILIGYGNWSRSTQMKHFMPTMNKGLRKQIHKKYDTITINECNTSKKCCDCNKDLEYYKDKENKKVFRLLVCSNCVSCENKKIVFRTRDANSSINIMKLTSCWIDSQTRPTAFHSHISSFTCSTIKEEQEKVRPS

SEQ ID NO: 102 - protein, 650 aa, unidentified
  source 1..650: note = Emiliania huxleyi virus 88
MPRNKKDDARVIKCPLDKILKPEIVSIIRDIVPVYNRIAISATLFLNLRIRQLVESRASSTEFAQFFQANKLVKYFMAVTSSTLGAGDLHAAFGTNFPSVIREDRPKAFSQVCMYICRNLEATATTNVWYHMKKRVCTVVKKTFGIPRDEWLHMSNEEKKNHKIHLKQVNVDVLSLEGDAFVSPEQYHDWISNLRSEMKVTHVTRQLLADTRGVDVDDPNVPGMVYYMKVKPYEFIYAIATLSSHLNSITGGGYALYPIRSSMVPKYLHLDELILADISPGISKGATNTAAGKKRTADGSELSNKDKLVARRMIVFKHVMTPKAFNKWENHLGWSVDTDGYGCSLHVKKKVNELPPSTHPSIHNGLIKIDAFKDKFSERYSINDLSIIGVDPGKREIVSAVDSMDYTSKYTFRKTHQDHLTGTVRHRKAMECTKPQSVFETEQDLATTSKKVVPVERFMDYATKLATSISSSSGFYEQGVYRKRKWKRHLRLQKAYSGIVNNLKMVSCHPNKPIVLAYGSWGMTTSGLPFKGLPPTIGIGMVRYLARFFPVVITPEYYTSTICPCCDSNVTRCTHVEQQRASPFFENGRLKEIRGLKHCPGCNTYFNRDNMGAWNIARNFTNMYQGMAPIRVRNAAQAAEQELTNRTEVD

SEQ ID NO: 103 - protein, 640 aa, unidentified
  source 1..640: note = Emiliania huxleyi virus M1
MDDSHPCLIDMLNQQQSNNIMESTAEPLINGQEMEQLNTCDQHQTDNGYMGSSMETQVMDMFPSSMDESLPESNSITSDDSSMSSNRSTQDALPSPIVLPDLTTTAPVLNRFYDSRLKDVYNMFIYPTETDSVGSATISSSTYSTTMEQQLKLTNKMNSLPQTNRPSLQTTSFLSSQFSVLGCTEREVEAAVDAQVGEKRKKIPFQRQTELQGNLRTYKIKMFPTPEQKDELKRTFGMARYAYNWAIAQMKEGKKPNNIQLRNEFRKLPLPQWASEKKKQVATRITAGAIKQAVDAVLTNLKKKKKDPSHHFEMTFRSKFANSEVVNIEKAANGGSLAKMQAYTSEHLPYRAGRKNALLYFGNNLKQAGPILIEDNPNVIDMFVAEGKPKEDGKILYDRRRNTYYYIYLYEQPALRDPDPGFSDKDIVCTDPGEVVFQTFYSPSGSHGELLLDFRQVLLERCKKLDILHSRVTKKEKMRRQGLLQGRKNYLIIRRLKQAWARERARLSAFVERAHYFAANYILKNYDIIIQPQMAVSKMVSDLNSKVARSMLTMSHYMYNRRLEWASTRYAGRYIVHANEPGTSKTCTNCGYWHADLGTSRTFKCPSCGIAVSRDLAGARNNCLHAVGTVIRNRQLSEKR

SEQ ID NO: 104 - protein, 520 aa, Acanthamoeba polyphaga mimivirus
MKEAVKNVKPKVPAKKRIITGSKTKKKVFVKKKPPDKKPLKKPVKKTVKTYKLKSIYVSNKDLKMSKWIPTPKKEFTEIETNSWYEHRKFENPNGSPIQSYNKIVPVVPPESIKQQNLANKRKKTNRPIVFISSEKIRIYPTKEQQKILQTWFRLFACMYNSSIDYINSKKVVLESGRINVAATRKVCNKISVRKALKTIRDNLIKSTNPSIMTHIMDEAIGLACSNYKTCLTNYIEGQIKKFDIKPWSISKRRKIIVIEPGYFKGNSFCPTVFPKMKSSKPLIMIDKTVTLQYDSDTRKYILFVPRVTPKYSVNKEKNSCGIDPGLRDFLTVYSENETQSICPIEIVVNTTKNEYKKIDKINEIIKTKPNLNSKRKKKLNRGLRKYHRRVTNKMKDMHYKVSHELVNTFDKICIGKLNVKSILSKANTVLKSALKRKLATLSFYRFTQRLTHMGYKYGTEVVNVNEYLTTKTCSNCGKIKDLGASKIYECESCGMYADRDENAAKNILKVGLKPWYKQK

SEQ ID NO: 105 - protein, 545 aa, Acanthamoeba polyphaga mimivirus
MAKTTNGKFWNDECEKVSDLLWKPNLIVNHKPQERINFKCDNKYITFNYYSNNVPINDQLKFKDIPQSNNTDKKNKLMDKLLKIEDSRYKKDIESNKTRKIKLTKEQLTAKHNTQLLKIQNKISQLDGFIRSRRIQLLPNDFQKNIFNIWFNETTVIYNRLLTEFTQYYIKFKEIASDNKELVKLLRTNEEFPLSLQKLRNLKIHDIINIYLEDQLIHKLETPFCVIANTIKEFVANVKGNLTKLAENKIDKFTFKHKNFNGKIRTISIDSKYASENSFYPDKLGKLLQTYEDLKGFSWKNVKHDFKIVYDKYNNKYYAHIPKYVYRNLNILNKKPITVMDPGERTFQTLYGLDHVISIGDNLRETISKRLLRIDNLKSKLDTPGSKKHNLKLNRKTRVRKCRYKKAIDRHHKKIEHLQRELHYKTAIYLCENYQRIMVTDFSSKKVSSKDGDLDAMSKRILGKISHYRFRQRLQQKCEEFNCQYLEVNEAWTSKTCCNCGYIHAKLGTSKIYDCPKCKQKRDRDVNGAINILLKNRKLVLMEDQ

SEQ ID NO: 106 - protein, 482 aa, synthetic construct
MVYNQDLSISNKTCGISDWLPPVRNKKISDIKSNSWFDIRRCLNPNIKTKFVPVNNKQIVLKEPNCIYLKYDLITLNPSSDHIRILREWFSLYAKIYNYAINLIRKMFYVNNKLIKSRLQCEIYDEIIIRKLSNDKTKLIESLKLSDRPPEFLLDYAISHACTYCNSQVKKYLKDSKNPYNKKTLRFRLWSLHKSRRVMHILPPKLCDKNVCNGLFLKLNPSKNIHDIYMFCVMSWDRLTGKFFVYKPVLKRLINIPKKQFTRCGIDPGVRTFLTLYSSERTYDVCDSTFYNNKIKRMWRKIDRINFLLHNRKKRKKRKNDRRLRKSLAKYYRKIRQRIKDLHTKTAKYLVSRYKEIYIGIYDTSKNLSDPRISFYEKRRIATLNHELFLTKLQQVADRFGSSVFAINEHMTTKTCSKCGRINEIGSSKVHKCACGMYAGRDENAAKNILKKGIKEHYLSAQSDKNVTEYNNKKSNNTNKET

SEQ ID NO: 107 - protein, 526 aa, synthetic construct
MFWNNQCAKLSKSLWIPSNIINLKSNKKKYFKCDNPNISFNYFTDGIKINKKIKFNDIKVTDNHDKKEKLRNKLIKTENTRYIKEQNNQKCKLNKQELNDKHKKFLDKIEIKVNQLDGFIRSRRIQILPNKKQQNIIQKWIYDTTSIYNKLVSSFNQIYDKCQKIVDNMNVENKFYHLGKIIKQNTEFPINFHKLRALKCSDFIQDYDRIPYCVIADIIKEFVSNIKSCITKISKGLITEFKFKPRKYNRIFSYIPLESHYTTVRGFYPSIFGKIKTNEKIFSWLDIKHDYKLIYDKYSNKYYIHVPKYVYRNDLTIKKPIAIMDPGARTFQTIYGLDHVVTIGDDLGSTFKKRLLKIDNLKSKLSKPGKYKHNKKLDRKTKVKKWKYKRAIDRHHKKMDHIQQELHYKTANYLCSHYDRVVVTNFSSKKVGSKKNDLDAITKRILGKLSHYRFRQRLLTKCEEYGSQYYEVDESFTSKTCCKCGNIHKTLGSSKIYDCIKCKNKIDRDVNASICILIKNKDILLK

SEQ ID NO: 108 - protein, 493 aa, synthetic construct
MVYIKNRIPIHYPNKNCGLPNWVPSINNYYDCLNTNSWFDMNICKNPNKSSVKRHLIDAPIVPNESYKQQMKSIKRCKSNKEPVFIYTRKIRIYPTDDQKCILEEWFWAATRMYNITVKYIRDFLFKDHKLTCPFASKDILINFPFRTVLKEDRDKLMKSMKNPIVGHLLDEIIAQVVSNYKAAISNILVGNIKKFKISTWKYIREKYILKIEKNLFSNGTFCSRTFKELKSSEPLINIQHTCTLQYNRDIGKYILLVPTHKETKSYVTNDLDCGIDLGVRTFATVYSRNNVVTIGNKFKKIDSCHKKIDKINELLDSDNNKCKLRKAKRKYYRRIKDIITDMHYKSAYYLVTNYDNIHIGKLSTKGILSKNNKKISRHTKRMVGVLSPYLFRQRLNHMAHKYGATVFEVNEYLTTKTCSNCGKINELEAKKVHKCKCGMKTDRDVNAAKNILKVGYQYNSTLHQVNDIDIIETDNHIVTFNKVIKEYEVFEV

SEQ ID NO: 109 - protein, 351 aa, synthetic construct
MVLKRWCGLYRRMYNLAIEKMLDTGRWHWKGHKVLTTYDNIPKNEKVRLLPTNIRKEAYREASTAMAKAPHKNTVKFKTLKRNGRSLRVFGAVSIVDTPDGKRIKLTDSQKHKQLSKELIGFDQKNLADIRIGSSSQIVYDGKWWLLSPFEVERSNVPKKHAVALDPGVRTFQTTYSPDGTSKKLGDQASCRIFRLMKYIDKLAVFKDKRTRGKRLRLERRVGNLVDEMHWKIARELATDYDTIIIPPFETQKMAKKWKNGRYRKINKTAVRMMLRLAHYRFRERLIHVANKLGTRVVICTEEYTSKTCGNCGVVNADLGSAKKFKCPRCHIHVDRDGNGARNILIKSVCC

SEQ ID NO: 110 - protein, 395 aa, synthetic construct
MRCHEISWPSGKLDQHLRNKFSIGKHNDIEQYISIHGNNIEPEQIIETTKMNTETRKKEPSRRYINPFFLGKLWLSECPKDFRARATFKAADAYKTSLTLWRKGLIRSFEMKFSTKRRELDRGYCFGIEKKVKFEDTGLKKRDGKLIITGIDGDIRFFEKPPINKEPVAGCELSKDSCGDYYLHVPIFRKKKERKGDNFVAIDPGGVVPWAFYASKGESGCLGTEMNERMSNVSKKISTIDKKLSGQPKGDKNSNDVIERRKLFRKKKRIRDHEHYRVINFLTDRYDGIILPKLKTKNISKALKSKANHDMFDISHYTFLRRVGERCIESDTILEHPGEQWTSKTCGRCGKSNLPGRERRGTFREYKCLYCGLHAHRDVHAARNIFMKWYIELKK

SEQ ID NO: 111 - protein, 432 aa, synthetic construct
MTLKPWWSNKSQQLTKIISWNPTNTNIFQNDTTIKKVLRTRKVKLNPTKQQKMMLMKFADAARYSYNIAIAAVNDKTHKANKIELQNAFSSLKRRDDDSYNQFFQKRRWLLATPQPIRKQAIFEAAKNFKAAFTKLKNKNIDHFKMKFKTKKHQRQHGFSLGIGSHLKHDNGVLTILPRSLGQMRYFGFLPFEGKPDAECRIQRDPYGDHWLLVPVYKTTKTQISKPIVAIDPGVRTPFACFRSDGDSKCIGEDMNAKLIDIKTRVTIVDRRISKCQDGSLKRKLREHRRVLFRKHQRVRDAFHWKIINDITDECSGVLLPPFETQRVSRMLKAKTNCSMLGISHYTFRMRMKGKCEEKGLLYDEPTEEYTSKTCGVCGLINYLLGSKKTFECFCGNVCDRDVHAARNILLKWLSSEAGARVAETFLASRSS

SEQ ID NO: 112 - protein, 416 aa, synthetic construct
MNIKNYHWFKNKKRKNINTDGWFNLKSINNKGFNDYDNIEKPINKFLCCKTIEIYPNLEQKNIILKWMDLFINMYNHTNYFINNNIYDFTNRKIKSNVKEILNFRKLRDNYIKNKKNETCKDEINKHMLDEAINHCVSKHKSCITNYFSKNIKFFRIRNMKKNRRKKILIIEKGLFSKKKNGFCLTKLQEIKSSESIGVNKTSILQYDKHLNKFTLFIPIEKNTTNIENRTKCGIDPGIRTFLTCYSKKEILEIGENCYEKYKNNYKKIDKTRELFDKKEINKKQFTKSINHNFDKIRNLTKDLHFKVSSYLCKKFNIITIGKISTSSIVLNTNNLNKISKRCMLSLSHFKFREILEYQCNKYNTQLNIVDESYTTKTCSNCKELNNIGSSKKYQCKSCNFKCDRDINASINIYLK

SEQ ID NO: 113 - protein, 408 aa, Feldmannia species virus
MKDKGVVLRCRKVRILPSEEQREILKSCFGIHRHIYNECVKAEKDGDIDGAGVRECYKWRKAFTTREDYYGQGKNWKDDCPSHTKQQAVEEFFKNKKTGLKLVSEGKIRRFDIGFKSRFKCRQETIPFEKFLIKKKDSTNPLSKSLVSTTYKGKSMDLRIMGKVPKVFKDRDDQGFTRTEMKITRTKTGKYYAIVSFEVPQTSRFPEIHGDMISFDPGCKTFQTYHSPDGTWGEIGTFEKQELLLRKADRLKSRLELEGKDKDSRWRRRTRRRMLNLFEKVRNRTADLHNKVCSWVVNTYRLVLVPEFKTSQMVSSKKLYSKTCRKMMTWSHYRFRSKLIAMAQKFTDVKVRLCNEAYTTKQCGSCGVINRSMTLNDRSFNCVSCGLSSSRDGHAARNVGLRSLRFLV

SEQ ID NO: 114 - protein, 291 aa, synthetic construct
MRWIYILKCEDNYFYVGETARLYRRFWEHTAGIGGLNTQIFKPEEIVAIYKVDTIGKFLEYNNNIIDKQANNLIDYNRWLITHFNDCAKYKDYFDKLEAENNIAECMMIHNKDNWEKIRGGKYTKFNCDYKFPINNNIKELPMCKCGVPCDIKKNDDKNYLFFRCAKKNMWENFKDDFDIEDEPCNFFMEYITDIEFRIDEKKRVANYKQNFVDLIKKSFWLNNVECEIDDECPECVSCNKYVWCDKNGEFKNNGIEYGNERRLLCRDCFVNKNEELSQKYSLTGKCLIKL

SEQ ID NO: 115 - protein, 198 aa, unidentified
  source 1..198: note = Organic Lake phycodnavirus 1
MTTEIKFNNQYVLSRMEENKLIKIKLIIQELKQIAPLLSDVELLQLYNKSISIHQGKIQGNGNFLENDILVGMLELNNIPYKKQVTINKSGIIVGFNEKKGKCYHIIDFVIGENIEVGKSITEYKVISCKTTCRERWTQDDWSYTFIPKLYILLTISDDYPPSTRFREDIQRKIITCIPKRKDDRIYKLKFENLIEQL

SEQ ID NO: 116 - protein, 344 aa, unidentified
  source 1..344: note = Organic Lake phycodnavirus 1
MVKYSCEKCGKEFTQKGHYTKHTTKKIPCVFESKIEEMIEKVLAKKINEIKTTEVILNEEIPENIIENIKFIDLFCGIGSFHYSFKKLGWDCVMSCDIDKAVKETYKSNYGILPLGDITEIEPKNITNYDILCAGFPCQPFSQCGQHKGFDDKRGTLFFNIMKFVDYHKPKVIILENVMGLLNHDGGKTFEKIKCDIETSNYSITYKVIKCSDYGLPQMRKRLIIVGVRNDTALINHIDKLLDLDEYKKETTLTELLGKNFEKKIAYTIRCGGKNSPIDDKHNWDGYMVDGKEYRLTKEDCLKIQGFSSDFKLCGNNKDQWKQLGNTIPTIFTEIIGLNLKKYL

SEQ ID NO: 117 - protein, 256 aa, synthetic construct
MNNSKMDLQIEAITTLLSSLAINETKIIKIQKWFRGCILRLKQLPLIMYKIKKYLKLQAFQFSTQNEDGRINSCIDEDEVIKLLIEKFGGKIKKPKIRMWYDILAFDYMYGWVPINIKTTTTITSDNTGNLAMCVYAYTNEILDINRDKSYENGKMSDILFNKLKNKKYNTNNKKDYYFIVLNKTDASDIIVNSVKGLTILTPNINNLPFQVCWDKNRKFKYENINKKIKLFIDCLQKPKPSWKETFMSNIRTLDL

SEQ ID NO: 118 - protein, 459 aa, synthetic construct
MVFYSCEKCGKQFSQKGHYNKHINKKLPCVNETKLKEIIGVVVNEKLNEINNNKEKIDLFISKDLEETINTEIPTMTTKHNLGQYFTTHNELKEKVFEFILNTPSNILEPSMGQGDLITFITDKIPSITFDMYEIDKKIKLLDKIQKDKINYGDFMTQTITKTYKTIVGNPPYVRTKKGNLYIDFTEKCYNLLEDNGELIFIVPSDFLKLTSASKLLNVMMTNGTFTHIFHNHNEKMFDNASIDVIVFRYCKNSLIDKKVLYNDKLLYITNSNGLITFGEEENNNSVLFQEYFDIYVGLVSGKEEVYKNEELGNIEVLNGEDKIDKYIYIENYPCDNEKINKHLSHHKKELIERGIRKFNENNWFEWGAPRNITTINTNLGKDCIYIYNLTRKPNVSFLGKVNYFGGGLIMLKPKKKCNLNNIVSYINSNTFKDNFMFSGRFKIGHRQICNSYIPSEYL

SEQ ID NO: 119 - protein, 402 aa, Deinococcus radiodurans
MKRLQAFKFQLRPGGQQECEMRRFAGACRFVFNRALARQNENHEAGNKYIPYGKMASWLVEWKNATETQWLKDSPSQPLQQSLKDLERAYKNFFRKRAAFPRFKKRGQNDAFRYPQGVKLDQENSRIFLPKLGWMRYRNSRQVTGVVKNVTVSQSCGKWYISIQTESEVSTPVHPSASMVGLDAGVAKLATLSDGTVFEPVNSFQKNQKKLARLQRQLSRKVKFSNNWQKQKRKIQRLHSCIANIRRDYLHKVTTAVSKNHAMIVIEDLKVSNMSKSAAGTVSQPGRNVRAKSGLNRSILDQGWYEMRRQLAYKQLWRGGQVLAVPPAYTSQRCAYCGHTAKENRLSQSKFRCQVCGYTANADVNGARNILAAGHAVLACGEMVQSGRPLKQEPTEMIQATA

SEQ ID NO: 120 - protein, 630 aa, Staphylococcus aureus
MNASSKRKIISQSEISKKIAVMNEEMQGFWANNSWDIRKCPHPSAIELSKNPALRNRWVRFERVKNLWLRTELKYFYFYHLNNGIWNAKTVWIRKGTVINKMLDFLDLKYPSITSITEVPIEKAMTEYRTYLTKRGVRITTTNYKITANQEKTPVKANSYYVTNLKQFMEFYENFYFDGEEWDKDVWDRRNLPLPDDKVNPTQYEYTINFKGFRNTYFKQLVKRYCKLRLNVDSFSYVSDIAQRLKEFFNFLDMKFKQVQRVHQLTRVEIEAYLSELNMMGIKPSTITGRISILEGLFSTLLRLEWDDVPSKILIYSEDYPKIPRAKPRFIDEFVLEQLNSHLDKLPEYIATMTMIVQECGMRISELCTLKKGCLLEDKDGDFFLKYYQWKMKKEHIVPISKEVALLIKVREDKVSEEFPDSEYLFPRKDGSPLKQETFRGELNKLAYEQNIVDKSGEIYRFHAHAFRHTVGTRMINNGMPQHIVQKFLGHESPEMTSRYAHIFDETLKNEFTKFQEKLVTNNGDVLDLDEDNEVDDVELQWFKKNINAQVLPNGYCRLPVVAGGCPHANACLDCTHFCTSKQFLPQHEEQLERTEELLAIAKDKQWQRQVETNSRVKERLEQIIGSLTG

SEQ ID NO: 121 - protein, 402 aa, Escherichia coli; identical to SEQ ID NO: 119

SEQ ID NO: 122 - protein, 520 aa, Acanthamoeba polyphaga mimivirus
MKEAVKNVKPKVPAKKRIITGSKTKKKVFVKKKPPDKKPLKKPVKKTVKTDKPKSIYVPNKDLKISKWIPTPKKEFTEIETNSWYEHRKFENPNKSPVQTYNKIVPVVPPESIKQQNLANKRKKTNRPIVFISSEKIRIYPTKDQQKILQTWFRLFAYMYNCTIDYINSKKVVLESGRINVAATRKVCNKISVRKAQKTIRDNLIQSTNPSIMTHIIDEAIGLACSNYKTCLTNYIERHIKKFDIKPWNMSKRKKIIIIEANFFKKGTFCPTVFPKMESSKPLTMIDKTVTLQYDSDTRKYILFVPRVTPKYSVNKEKNSCGIDPGLRDFLTVYSENETQSICPIEIVVNTTKNEYKKIDKINEIIKTKPNLNSKRKKKLNRGLRKYHRRVTNKMKDMHYKVSHELVNTFDKICIGKLNVKSILSKANTVLKSALKRKLATLSFYRFTQRLTHMGYKYGTEVVNVNEYLTTKTCSNCGKIKDLGASKIYECESCGMYADRDENAAKNILKVGLKPWYKQK

SEQ ID NO: 123 - protein, 278 aa, Methanocaldococcus jannaschii
  source 1..278: note = Methanocaldococcus jannaschii DSM 2661
MLFKFEGDKIKIITAPRKFITINLVVSDYQKKFIEEWKNGNFKIGEVIIKKDSIIIPFKKVVNPKNFEHIMTIDINEKNITYSIFDKDGNVIKTTRLDVYKLKRIHENFSKKREKIQKKLSNKPMKLKTLMEKYSGREKRKVEDYLHKISKFLISEALKYNVKILMEDLTNIREAVNKKSKNFRRRLNRWNFSKLQFFIEYKAKWDGLDVEYVNPSRTSKLCPICGCKLDPNGQRLLKCNNCNLVFDRDVVATFNLFKKSQDVGSFRSPERSLMKSSY

SEQ ID NO: 124 - protein, 306 aa, Aquifex sp.
  source 1..306: note = Aquifex aeolicus
MKVYKRLYELLGRGYKPLKSARLIKRGKDYYIGITLQKAVKEKKIKKPRYVINVDLNVQRNLACIGIFEVDWEKRESKLYGIKFVNGKLLRLVYKRDYLFEEIRKKQRQTGRSPQVGDNSRLWKKVNNLNRDIALKVAKEISDIAREFSEKGEVIVVFEKLKGLRGRKGRSKKLNRKINFWMRRKIQERVKELGLEEGFGLDFVYPHYTSKKCSKCGYEGERFSPSGSKALFLCKKCGYVVNADVNAVFNQHFLYLSHLLNGGGKARPVVRVGTSLKSPSREGHNLSGVPKATTTFFYISSLLSTF

SEQ ID NO: 125 - protein, 527 aa, Acanthamoeba polyphaga mimivirus
MKEAVKTVKPKVPAKKRIVTGSKTKKKVFVKKKPPAKKPPDKKPLKKTTKKTVKKVINRPKSIHIPNKDLKIFKWIPTPKKEFTEIETNSWYEHRKFENPNKSPIQTYNKIVPVVPPESIKQQNLANKRKKTNRPIVFISSEKIRIYPTKEQQKILQTWFRLFACMYNSSIDYINSKKVVLESGRINVAATRKVCNKISVRKALKTIRDNLIKSTNPSIMTHIMDEAIGLACSNYKTCLTNYIEGQIKKFDIKPWSISKRRKIIVIEPGYFKGNSFCPTVFPKMKSSKPLIMIDKTVTLQYDSDTRKYILFVPRVTPKYSVNKEKNSCGIDPGLRDFLTVYSENETQSICPIEIVVNTTKNEYKKIDKINEIIKTKPNLNSKRKKKLNRGLRKYHRRVTNKMKDMHYKVSHELVNTFDKICIGKLNVKSILSKANTVLKSALKRKLATLSFYRFTQRLTHMGYKYGTEVVNVNEYLTTKTCSNCGKIKDLGASKIYECESCGMYADRDENAAKNILKVGLKPWYKQK

SEQ ID NO: 126 - protein, 443 aa, Lactobacillus crispatus
MKRMSSLAYHFGLKVRFYPSSKQKKIIKLNYDAQRFVYNSYVGRNRNNYRARGYLAVRQYQVMPFAFSVLNNYETKLAEEVVANNELLAKPKNIRDAYSFLRVKEIDSLAIANAIQNYQKAWNNYRKLGHGIPTFHKKRSDWSYQTNCQYPKQTEAFLDNGTARFIDAKHVKLPKLGVVRIASFRKLIKERFLNHIPTRIGTVTIKKTADDQFYLSMQLGSDIAFVKELPKTQSQIGIDLNLDNFLTASNGAMVANPRFYRKTKKKLAHAQRVLSRRQRRAKKEGRNLRLAKNYQKQRLAVAKLHDKIRRQRNDFLQVLSTALIKNHDLVVAEELRSKNLLKNHALSQAISDVGWRSFLNMLAYKADLYGKEFKTIDPKYTTQRCHQCGSIMGQNGYKKLTLKDREWTCPICQTHHIRDWNAAVNILEKGLGNWQNSKIKKAA

SEQ ID NO: 127 - protein, 283 aa, Yersinia ruckeri
MSTFYAVRRLCLVLGLHYSGYYQWLKRPQSLRARQDERQTGLIKQLWLESGTVYGYRKIWLDMKDLGERAGRNRIARLMRLAELASQTGYRKRRYYGGGKTSVANPNYLERQFIVPTPNTHWVSDMTYIRTHEGWLYLAVVLDLFSRRVIGWSMGGRMTAELVMDALLMAVWRRKPTAKVLIHSDQGSQYTSERCQHFMTAHNLKSSMSRRGNCHDNAVAESFFQLLKRERIKKRIYKNREEAKADIFDYVEMFYNAKRRHSACEDQPPAVYESAWFMRHATV

SEQ ID NO: 128 - protein, 263 aa, Bacteroides fragilis
MKSSMAMIKHKKELVEYARHLKLPNLAEHVGVILHEAQEKQLTYSEFLANCLAREIQGRERKSYLTRLKLSGLPAKYDLDLYDYDRAEGMDNRRLRELRELVWVNQAYNLLLVGPSGTGKTFIAAGLVYEAVKAGYEAYLMTLEELLTCLKTKEVSAHAMKTYKRIMKARLLAIDDATLFPLKREEAVLLFKLVNDFQERTSLIITANKALTRWLETLEDEAVTAALLDRLLYCCEIIRLGGTSYRMQNRKTIFSNQNTDIGT

SEQ ID NO: 129 - protein, 362 aa, Cohnella sp.
  source 1..362: note = Cohnella zeiphila
MQITFQFRLYPTPEQEQQMNRTLDLCRKLYNRAKEQRDTANNQEGKTVTNTMQQNELPAFKKEFPAYKEVHSQVLQDCLRRLDHAYQRFFRGEAGFPHYKSADRYLSFTYPQPGAVTKTFAKGGYVYLPKIGLVRMRAHRPFDRANVTQINVKRYADGWMAGIGVKVETAEASQTMETAVGIDVGLRTFPALSDETKVNSPRHLRKAEKRLRRAQRRLSRKQRGSRNRQKAKRKVARIHQKVARQRKDFLHKQSHRIVRSHDLIAVEQLKVKNMIRNRRLSKSISDAGWRKFITYLSYKAERQGKRFVQVPAHGTSQTCVCGANVPKTLATRLHECKRCGLVQDRDIVSAKVILQRALEIAA

SEQ ID NO: 130 - protein, 403 aa, unidentified
  source 1..403: note = Moorena sp. SIO4A3
MKARYKYRFYPTDQQKQDLARLFGCVRVVWNDALAICKKYKKVLKSGDLQKLVITQAKKTEERKWLSEVSVVPLQQSVADLGVAFKNFFESHNGKRKGRKVRSPRFKKRSNNQSARLTRRGFSIQGDGVYLAKIGVVKPIWSRELPSDPSSVTIILDCANNYFLSFVVDIKPVQVDAKNQSIGIDLGIKTFAVMSDGGHVKSPDYSKLDRKLRKQTRKFARQAKGSNRREQTRIKIARLNKKIANKRKDFLHKFSTRIVSENQVIVLEDLNVSGMVKNRKLARAISQQGWSEFRSLAQAKSDKYGRDFRVISRWEPTSQICSDCGFKWGKLDLSVRSILCVNCGTEQDRDINAARNIEKVGMGHRHDSKWARRGDKTVETVGHATRTAAHPDDLSRIAVLLGR

SEQ ID NO: 131 - protein, 424 aa, Streptomyces sp.
  source 1..424: note = Streptomyces boncukensis
MQLRYNYRAYPVASQRRALARAFGCARVVWNDCLRDRKEAHAAGLPYMKSAELSRLRITQAKHTEERAWLADVSAVVLQQSLRDLDTAYKNFFDSVKGKRQGRKTGPPRYKSKKDTRQSIRLNTNAFSLQGDGTVYVAKVGNLNVTWSRRLPAAPTSLTVTKDSSGRYVLSFVVDTEPDILPEVEAESGIDLGLSAFAVLSDGRKIDSPRFLRRAEKKLERLQREHSRKAKGSKNRAKARIKVARQHAKVADRRRDWHHKASTQIIRDNQAVYVEDLAVSGLGRTRLAKSVHDAGWSAFTAMLEYKAVKHGRTFAKVDRTFPSSQVCSVCGFRDGPKPLHVRAWTCGECATVHDRDHNAARNVLFEGRRIVATGRVETLNACGVPVRRAHVPAQRGETGSPRKGQTTQAGITGLQAREHVKVAV

SEQ ID NO: 132 - protein, 404 aa, Symploca sp.
  source 1..404: note = Symploca sp. SIO2C1
MKARYRYRIYPTVGQQMALARLFGCVRVVWNDALAFCIAQYQGGKKKPKNAELQKQFITQAKRIIERTFLSQVSCVPLQQSLNDLEQAYQNFFKSCKGQRKGKPVKPPRFKKRHAKQSARFTQRGFSLKGDCLKLAKIGKLKIAWSRPLPAEPTSVTIIKDAAGRYFVSFVVEIVSEVLPNTEKSVGIDLGISTFATLDNGQKIDAPKPLKKRINRLRRLSKNLSRKTRGSKRYERARKRKAKLQAKLKDTRTDFLHKLSTQIIRENQTIVLEDLNVAGMVKNRKLSKAISDLGWRQFRTYLEGKVQKYGREFRVISRWEPTSQTCSECGFRGGKLDLSIREWTCLNCGITHDRDVNAAKNIKLAGGHSESLNGRGGRRKTSAKGAAAREASTHREYVQLSLFG

SEQ ID NO: 133 - protein, 630 aa, Staphylococcus aureus; identical to SEQ ID NO: 120
  source 1..630: note = Staphylococcus aureus strain Mu50

SEQ ID NO: 134 - protein, 442 aa, Lactobacillus kefiranofaciens
MKRMSSLAYHFGIKLRFYPSSKQKKLIKLNYDAQRFVYNSYVGRNRSSYHAKRYFLVKQSKTMPFAFSILNRYETKLAETVVANNELLAKPKNIRDAYSFLRIKEIDSLALANAIQNYHKAWNNYRKIGHGIPTFHKKRSDWTYQTNCQYIKQEEAYLDNGTAKFIDAKHVKLPKLGVVRIAGLRKLIKERLFKHIPTRIGTVTIKKTAEDQFYLSMQSGSDLAFVKELPKTQNQIGIDLNLDNFLTNSNGAMVANPHFYRQAEKKLAHAQRVLSRRQRRAKKARRNLRTAKNYQKQRLIVAKLHDRIRRQREDFLQVLSTALIKNHDLVVAEELRSKNLLKNHALSQAISDVGWRKFLNMLAYKGRLYGKEFITIDPKYTTQRCHACGTIMGHNGYKKLKLKDREWTCPICHTYHIRDWNAAVNILQKGLGSWQNPKKQAT

SEQ ID NO: 135 - protein, 421 aa, Helicobacter suis
MLKAIKFRIYPTIEQKTLIHKHFGCARVVYNYFLAYRQKQYAQGIRENYFSMQKALTTLKKQEAYAYLSECNSQSLQMALRQLTTAFDRFFSKLADYPRFKSKKHSKQSFCVPQHLEMDLGNNQVKLPKFKEAIKAKFHRHLPTNSIVKQGFISCVADKYYLSISYEDNEPEPKPTTIRKAVGLDMGLESLVIASNGVFYPYKKFFQNLQTKLTKAQRRLSKKLKGSNNRKKQAKKVAQIHASIRNSREDYLHKISNEITNQYDLIAVETLKVRNLVKNHKLAKSIANASWSRLISLLEYKAGWKGKTLIKIDQYFPSSQICSTCGSNTGKKPLPIRNFIYPCCQTYHHRDLNASINIRNYALGMLDERHAIKVDKTRVGITRSYACGDSANGAVTKYGYILDTASYGSLKQEAHPSLAGG

SEQ ID NO: 136 - skipped

SEQ ID NO: 137 - protein, 6 aa, synthetic construct
GGSGGS

SEQ ID NO: 138 - protein, 9 aa, synthetic construct
GGSGGSGGS

SEQ ID NO: 139 - protein, 12 aa, synthetic construct
GGSGGSGGSGGS

SEQ ID NO: 140 - protein, 15 aa, synthetic construct
GGSGGSGGSGGSGGS

SEQ ID NO: 141 - protein, 18 aa, synthetic construct
GGSGGSGGSGGSGGSGGS

SEQ ID NO: 142 - protein, 21 aa, synthetic construct
GGSGGSGGSGGSGGSGGSGGS

SEQ ID NO: 143 - protein, 24 aa, synthetic construct
GGSGGSGGSGGSGGSGGSGGSGGS

SEQ ID NO: 144 - protein, 27 aa, synthetic construct
GGSGGSGGSGGSGGSGGSGGSGGSGGS

SEQ ID NO: 145 - protein, 30 aa, synthetic construct
GGSGGSGGSGGSGGSGGSGGSGGSGGSGGS

SEQ ID NO: 146 - protein, 33 aa, synthetic construct
GGSGGSGGSGGSGGSGGSGGSGGSGGSGGSGGS

SEQ ID NO: 147 - protein, 36 aa, synthetic construct
GGSGGSGGSGGSGGSGGSGGSGGSGGSGGSGGSGGS

SEQ ID NO: 148 - protein, 39 aa, synthetic construct
GGSGGSGGSGGSGGSGGSGGSGGSGGSGGSGGSGGSGGS

SEQ ID NO: 149 - protein, 42 aa, synthetic construct
GGSGGSGGSGGSGGSGGSGGSGGSGGSGGSGGSGGSGGSGGS

SEQ ID NO: 150 - protein, 45 aa, synthetic construct
GGSGGSGGSGGSGGSGGSGGSGGSGGSGGSGGSGGSGGSGGSGGS

SEQ ID NO: 151 - skipped

SEQ ID NO: 152 - protein, 6 aa, synthetic construct
GSGGSG

SEQ ID NO: 153 - protein, 9 aa, synthetic construct
GSGGSGGSG

SEQ ID NO: 154 - protein, 12 aa, synthetic construct
GSGGSGGSGGSG

SEQ ID NO: 155 - protein, 15 aa, synthetic construct
GSGGSGGSGGSGGSG

SEQ ID NO: 156 - protein, 18 aa, synthetic construct
GSGGSGGSGGSGGSGGSG

SEQ ID NO: 157 - protein, 24 aa, synthetic construct
GSGGSGGSGGSGGSGGSGGSGGSG

SEQ ID NO: 158 - protein, 27 aa, synthetic construct
GSGGSGGSGGSGGSGGSGGSGGSGGSG

SEQ ID NO: 159 - protein, 30 aa, synthetic construct
GSGGSGGSGGSGGSGGSGGSGGSGGSGGSG

SEQ ID NO: 160 - protein, 33 aa, synthetic construct
GSGGSGGSGGSGGSGGSGGSGGSGGSGGSGGSG

SEQ ID NO: 161 - protein, 36 aa, synthetic construct
GSGGSGGSGGSGGSGGSGGSGGSGGSGGSGGSGGSG

SEQ ID NO: 162 - protein, 39 aa, synthetic construct
GSGGSGGSGGSGGSGGSGGSGGSGGSGGSGGSGGSGGSG

SEQ ID NO: 163 - protein, 42 aa, synthetic construct
GSGGSGGSGGSGGSGGSGGSGGSGGSGGSGGSGGSGGSGGSG

SEQ ID NO: 164 - protein, 45 aa, synthetic construct
GSGGSGGSGGSGGSGGSGGSGGSGGSGGSGGSGGSGGSGGSGGSG

SEQ ID NO: 165 - protein, 4 aa, synthetic construct
GGGS

SEQ ID NO: 166 - protein, 8 aa, synthetic construct
GGGSGGGS

SEQ ID NO: 167 - protein, 12 aa, synthetic construct
GGGSGGGSGGGS

SEQ ID NO: 168 - protein, 16 aa, synthetic construct
GGGSGGGSGGGSGGGS

SEQ ID NO: 169 - protein, 20 aa, synthetic construct
GGGSGGGSGGGSGGGSGGGS

SEQ ID NO: 170 - protein, 24 aa, synthetic construct
GGGSGGGSGGGSGGGSGGGSGGGS

SEQ ID NO: 171 - protein, 28 aa, synthetic construct
GGGSGGGSGGGSGGGSGGGSGGGSGGGS

SEQ ID NO: 172 - protein, 32 aa, synthetic construct
GGGSGGGSGGGSGGGSGGGSGGGSGGGSGGGS

SEQ ID NO: 173 - protein, 36 aa, synthetic construct
GGGSGGGSGGGSGGGSGGGSGGGSGGGSGGGSGGGS

SEQ ID NO: 174 - protein, 40 aa, synthetic construct
GGGSGGGSGGGSGGGSGGGSGGGSGGGSGGGSGGGSGGGS

SEQ ID NO: 175 - protein, 44 aa, synthetic construct
GGGSGGGSGGGSGGGSGGGSGGGSGGGSGGGSGGGSGGGSGGGS

SEQ ID NO: 176 - protein, 48 aa, synthetic construct
GGGSGGGSGGGSGGGSGGGSGGGSGGGSGGGSGGGSGGGSGGGSGGGS

SEQ ID NO: 177 - protein, 52 aa, synthetic construct
GGGSGGGSGGGSGGGSGGGSGGGSGGGSGGGSGGGSGGGSGGGSGGGSGGGS

SEQ ID NO: 178 - protein, 56 aa, synthetic construct
GGGSGGGSGGGSGGGSGGGSGGGSGGGSGGGSGGGSGGGSGGGSGGGSGGGSGGGS

SEQ ID NO: 179 - protein, 60 aa, synthetic construct
GGGSGGGSGGGSGGGSGGGSGGGSGGGSGGGSGGGSGGGSGGGSGGGSGGGSGGGSGGGS

SEQ ID NO: 180 - protein, 5 aa, synthetic construct
GGGGS

SEQ ID NO: 181 - protein, 10 aa, synthetic construct
GGGGSGGGGS

SEQ ID NO: 182 - protein, 15 aa, synthetic construct
GGGGSGGGGSGGGGS

SEQ ID NO: 183 - protein, 20 aa, synthetic construct
GGGGSGGGGSGGGGSGGGGS

SEQ ID NO: 184 - protein, 25 aa, synthetic construct
GGGGSGGGGSGGGGSGGGGSGGGGS

SEQ ID NO: 185 - protein, 30 aa, synthetic construct
GGGGSGGGGSGGGGSGGGGSGGGGSGGGGS

SEQ ID NO: 186 - protein, 35 aa, synthetic construct
GGGGSGGGGSGGGGSGGGGSGGGGSGGGGSGGGGS

SEQ ID NO: 187 - protein, 40 aa, synthetic construct
GGGGSGGGGSGGGGSGGGGSGGGGSGGGGSGGGGSGGGGS

SEQ ID NO: 188 - protein, 45 aa, synthetic construct
GGGGSGGGGSGGGGSGGGGSGGGGSGGGGSGGGGSGGGGSGGGGS

SEQ ID NO: 189 - protein, 50 aa, synthetic construct
GGGGSGGGGSGGGGSGGGGSGGGGSGGGGSGGGGSGGGGSGGGGSGGGGS

SEQ ID NO: 190 - protein, 55 aa, synthetic construct
GGGGSGGGGSGGGGSGGGGSGGGGSGGGGSGGGGSGGGGSGGGGSGGGGSGGGGS

SEQ ID NO: 191 - protein, 60 aa, synthetic construct
GGGGSGGGGSGGGGSGGGGSGGGGSGGGGSGGGGSGGGGSGGGGSGGGGSGGGGSGGGGS

SEQ ID NO: 192 - protein, 65 aa, synthetic construct
GGGGSGGGGSGGGGSGGGGSGGGGSGGGGSGGGGSGGGGSGGGGSGGGGSGGGGSGGGGSGGGGS

SEQ ID NO: 193 - protein, 70 aa, synthetic construct
GGGGSGGGGSGGGGSGGGGSGGGGSGGGGSGGGGSGGGGSGGGGSGGGGSGGGGSGGGGSGGGGSGGGGS

SEQ ID NO: 194 - protein, 75 aa, synthetic construct
GGGGSGGGGSGGGGSGGGGSGGGGSGGGGSGGGGSGGGGSGGGGSGGGGSGGGGSGGGGSGGGGSGGGGSGGGGS

SEQ ID NO: 195 - protein, 32 aa, synthetic construct
LEPGEKPYKCPECGKSFSQSGALTRHQRTHTR

SEQ ID NO: 196 - protein, 48 aa, synthetic construct
TCGGGATCTGAGACGCCTGGGACCTCGGAATCGGCTACGCCCGAAAGT

SEQ ID NO: 197 - protein, 54 aa, synthetic construct
LEPGEKPYKCPECGKSFSQSGALTRHQRTHTRPECGKSFSQSGALTRHQRTHTR

SEQ ID NO: 198 - protein, 86 aa, synthetic construct
LEPGEKPYKCPECGKSFSQSGALTRHQRTHTRLEPGEKPYKCPECGKSFSQSGALTRHQRTHTRPECGKSFSQSGALTRHQRTHTR

SEQ ID NO: 199 - protein, 20 aa, synthetic construct
PKKKRKVEASSPKKRKVEAS

SEQ ID NO: 200 - protein, 363 aa, Homo sapiens
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 201 - protein, 427 aa, Homo sapiens
MSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGEALLEEALRLGADVVGAIPHFEFTREYGVESLHKTFALAQKYDRLIDVHCDEIDDEQSRFVETVAALAHHEGMGARVTASHTTAMHSYNGAYTSRLFRLLKMSGINFVANPLVNIHLQGRFDTYPKRRGITRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR

SEQ ID NO: 202 - protein, 161 aa, unidentified
  source 1..161: note = Bacilus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 203 - protein, 178 aa, Escherichia coli
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 204 - protein, 182 aa, Saccharomyces cerevisiae
MQHIKHMRTAVRLARYALDHDETPVACIFVHTPTGQVMAYGMNDTNKSLTGVAHAEFMGIDQIKAMLGSRGVVDVFKDITLYVTVEPCIMCASALKQLDIGKVVFGCGNERFGGNGTVLSVNHDTCTLVPKNNSAAGYESIPGILRKEAIMLLRYFYVRQNERAPKPRSKSDRVLDKNTFPP

SEQ ID NO: 205 - protein, 168 aa, Staphylococcus epidermidis
MKKNHDYMRLAINEAHKAKALGEVPIGAVIVKEGQVIARAHNLRETLQQPTAHAEHIAIERASEVVGSWRLEECTLYVTLEPCVMCAGTIIMSRIPKVVYGATDPKGGCSGSLMNLLEQPQFNHRTTVEKGILEEECAELLRSFFKEIREKKKAEKQGKIQKDINLLK

SEQ ID NO: 206 - protein, 1733 aa, unidentified
  source 1..1733: note = Moloney murine leukemia virus
  VARIANT 537: note = X can be any amino acid
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTLSIKSKPPKPQVLPDSGGPLIDLLTEDPPPYGVQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEAGKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIENKYRLHETSKEPDVPLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHPSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVTKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 207 - protein, 641 aa, unidentified
  source 1..641: note = Moloney murine leukemia virus
QAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLL

SEQ ID NO: 208 - protein, 142 aa, Mus sp.
HLATGKVTHSFLHVPDCPYPLLGRDLLTKLKAQIHFEGSGAQVVGPKGQPLQVLTLNLEDEYRLHETSAEPEASPGSTWLSDFPQAWAETGGMGLAVRQAPLIIPLKATSTPVSIKQYPMSQEARLGIKPHIQRLLDQGILV

SEQ ID NO: 209 - RNA, 1527 nt, synthetic construct
  source 1..1527: mol_type = other RNA
atgataaggaataaggctttcgtggtcaggctgtacccaaatgcggctcagactgaactgattaaccgcacgctgggtagcgcaaggttcgtctacaaccacttccttgcccgtcgcattgcggcctacaaggaaagcgggaagggactgacctacgggcaaacgagtagcgaactgacccttctgaagcaggctgaagaaacctcctggctctcggaagtagataagtttgctttgcagaactcgctgaaaaaccttgagaccgcgtacaagaacttctttcggactgtgaagcagtccggtaaaaaggtaggattcccacgtttcagaaagaagcgcacgggagagtcctaccggactcaattcaccaacaacaacatccaaattggggaaggtaggctcaaacttcctaagctgggatgggtgaaaaccaagggccagcaggatattcaagggaagattctgaatgtcactgtgcgccgtattcacgaaggccattacgaagcgtccgttctctgtgaagtcgagattccctacctgcctgcggctcccaagtttgcagcgggtgtggatgtcggcatcaaggattttgccatcgtgaccgatggcgtgaggtttaagcatgaacagaatccgaaatattaccgctccaccctgaaaagacttcgtaaagctcagcaaaccctgtccagacggaagaagggcagcgcacgttacgggaaagcgaaaaccaagctggctcggattcacaagcgcattgtcaataagcgtcaggatttccttcacaagctcaccacctccctggtgcgtgagtacgaaatcatcggaaccgaacaccttaaacccgacaacatgcggaaaaatcgccgccttgcactgagcatcagtgatgcgggctggggtgagttcatccggcagttggaatacaaggcagcgtggtacgggcgactggtatctaaagtcagcccctactttccatctagccagttgtgtcatgactgcggattcaagaatcccgaagtgaagaatcttgccgtccgtacatggacttgcccgaactgtggggaaacccatgaccgagacgagaacgctgcgctgaacattcggcgtgaagcgttggtggctgcgggaatctcagacaccttaaacgctcatggaggctatgtcagacctgcttcggcgggcaatggtctgcgaagtgagaatcacgcgactttagtcgtgtgaggttcaagccggcaatcacgcccgagttgctgcccgagccgtcgcacgccagcaccttgacggcgatcagcgacacgcccttggcgacgccgtaggtgctgctgcccaccgtgccggcgacgtgggtgccgtggccgttgcagtcgctatcgttgccgtcgcccgactggttggtgccccacacggcgcggccaccgaacgccgtatgactagtgttgatgccggtgtcgatgatgtacgccttcacgccgctgccggtggtgccataggtgtagctgctgtcgagcggcaggctgcgct

SEQ ID NO: 210 - RNA, 1674 nt, synthetic construct
  source 1..1674: mol_type = other RNA
atgccaatttttgccgccaagaaaagacaagctacactgaagaagaagcttgttctccagcagggtgatgaattgaagaaggagagtctcaacatcccgagctgggcgccatttaagctgattgatcacggacaggccgcgaatgaccaggtgcttattcctcagaagccggaggttccgggtgacgcgcttaaggatcattggactccaaagaccgtaaggatatatcccactagcaaacagaaagagatactacacttatggcttacggctacaagacatatgtacaaccttgctgtggccgagagaattcgtttgggaaaggaggccaaggaaaacgggacaaaagtcaagatgaagctcaagcacctacgcgaagttatgcccttaaagaagaactttgattacgagcgcgacccgaagctcacctatttgaagaacgtaccgtttctaatcaaggacaatggcctacgtgatttcgacaaggactatcggaccaacctggccaaatctaaaaacacgctgagacccttcaagatccactttaagaaaataaaggataagcagagcatgacagtggagaaaaaagtatggggcctcagccgtggcatataccacgacgtattccggaaaggtgcatttaggagtaccgagccactgccagaacaactcccacacgattcaagactaatacgcgagcatggaaaatggtttttagtcatcccatatgagacaagcggcctcatgccgaagaaagaggctatcaatgcagttgccgtggatcctggcgtcagaacattcctaactacctacgacagcagccaaagggtgctagagatcggtgaggatgggtatctgaagcttcagcacctggaccgcagattgcgatcccttcagtcagagatccacaagcttcgtagcaagggaattgtgctatccaaggaagaccgaaaaaggaaaatacgtaaccgcaggactgtaatcagcaggctttatcagcggaagaaaaacctggctaacgatgttcacagaaaggcatcccatttattagcgagcaaatacaattacattctgattccggtactcaatttccataacttcaaaaaaacaaaccgcagagttaaggcgaatctcgcaaatatgaagcactgtctcttcgtgcagcgcctgaagaacaaagcttctcactttgaaaataccacagtaatcgaagtggatgaggaatttacgacaaagacgtgctccttgtgtggtgtccgcagtaatatcggtgcgtccaaagtgtttaagtgcgataattgtgacgcgactttcgatagagaccacaatgcggccaaaaatatccttttgaagtaccttggcgagatagattagtatgctatatgtagtgcttcctgtgtatgctgcaatgaatcgatgtttcatgagttaattttgtttttaccttttcgggaaatgctggcaatctaattaatcactgatctggcatactaagttatgcagccatctcttctgagaacggttcgtccgtcaaatgtataaatacagacgccatcgctctgttgtttgcactttcatatcttcctttagacctcgagcctggcttcttttcgaagttggtcctcgggtccccgccagctgcttagcagaggtcgtgaacatcatgaggcgatg

SEQ ID NO: 211 - RNA, 1760 nt, synthetic construct
  source 1..1760: mol_type = other RNA
atgaccacctgtactcaatatcaggctaagaagagaaaaagatcaaatagtaagacaaccaaaccatattggaatgaggatgcggctcatatttcccagcaatggggcttgaatacgattctaccaacggcggctaaaaatcttagtcagaaaatcacatctgactcgtggtttagcgtggttaaacatccacaggtgccggttatcaatgactcattgaacttgccgcttagcagtggtgtcaaagaagagaataccagggctcgcaaaattaaggtctatccgaccaattcccagagagaaacgctcaaaaagtggtttaggtgccagcgttacatatacaatagagcgctgaagctgcaccgcgatggcatggcaatgaccataaaaggtctacgccagactttgcttaacagagatactattattctagaacctcaggagcagtggcttaacgactacagttacgatttgaaagacgaagccctttgtgatttggtcaagaactacagttataatatggcaaagtataagcagactgggatgcctttgaagttgaggttcaagtcaaagaagacaccgattcaaactctttcagtgttggaaaagcactggaacaagagaacgaagtcgttttggagcgaagtattctcgctctcgaattttcgtggtacagagccgctcccagaggaattgccaggagattcgagggttcaaaggacaaggaacaacaaattctacttggtagttccacttgctggtgaggaaatcgctagaaaaagtcccgcagaaaagtttatatttattgatcccagggtgagaactttcctaacaggatatgactctaagcagactgtggtcgagatggggaaggacgcggttgtacgcatcgagaagcttaagcgccgacgacggcaattccagtcaaaacttgccaagctcaggaagcacaagaagaggcagaaccatcggaaagccttacataggctcgacgaaaagattcagcatattgttcgtgatatgcataagaaaacagcctcttatttgtgtaaaaactacgaaaacatctttctccccaaattgaattaccatcaatgcactagactgacccgaaagacaaggtccagcatggcaaccattgctcattgttgttttcacgatcgcttgacgatgaaggcagagcagttccatagtactagtgttcatgaagttgaggaggattacacgtcgaagacttgttctagttgtggcaatttaaaagatgatctaggatctaataaaatctacagctgttcaagttgctcctcggttttcgatagagacttcaacgcagcaaagaacatcatgctcaagtatatctgcgagcatttgatggcaagtggtggatcttccattacctctgcttcaagtggaggaaatggtggattcgccatgatgggtcctggccccttcgtacgttagtacgttgtaagtcccttgtggattctcccaggcgctacccgcggaacgtattactaagggtatatatataataaggataatatataaagtgctgcaaaagatgaggctcgaatttttcacaagttatcggtcccaacgacctaccagttaagaaagaccaattaaggaatatggttacttccagagctgtcacttttatcaataattctacacctgctactttggtgaaaaaagagattgaccttgataaatgtcatgatgaaaatcaaattgttatacaggtttatgctgccagtttg

SEQ ID NO: 212 - RNA, 1080 nt, synthetic construct
  source 1..1080: mol_type = other RNA
atgatcgtcccggcaacggtgctcgccgccgcagttgggaagaggaaacgaggcaccgggtcgtctcgtaaccagcagcggcaggcgaagcgtgtggcccgtcgagaacggaaccaggctgccgcgaagaagcgcaagacgtcgtcgggggctgtcaccacgaagcgcacgccgaagacaacaccgcgcaagcgcaagaccccggtgaagcccaagccgacgtggacgttcacgactgtcaagatgaagctgcggacgttctgcaaggacgagggtgtagcgctcgcttgggactcggtgctgagggacatgaacaagatggtggcggaggcttacgtgctggccgccgtccacgtggttcgctgctgtgagcaaggtttcgacatccccatcttggcgcgttccttctaccagcagtgcttgtcggccgtgagtggggggaacctgcacgatcgacggacgaccatgactgaagagttccgcgactcggtggcgatctacagcgcgtggcaaggcgagagcctgatccccaacagggacgccatcaatcgtggttgggcacaggtggcagccgagcgcatggcccatgaaacccagaaccatctccagctcaacttttaccggcgcttcaaacaatacattacgctccgctacgggctggctggccgcgatcgctaccttgtgctcaagggtatcctcgaccccgagtacgacggagacaacgagatcgtgctggagtacagagaattgatcccccgcaatgatgaaggagcgagctagcgagctactcccgcttgtgcacatgttcctcactttcgtgaaggaccacaaccgcgagcacgaaggagaaccgggcttcgagatggcaaggacgtactcgctactaccaacgaaacggggcttcgtctgctctcacttcaccatgtcctctcgcggcttgcgggcgatgctggttcgaagtggatatgatgttccggaagagggtagcgactggaacgctgtcgcggacagcatgtggcggcggctcttcaacacgaaccttgtcgagacgtccaagccggacgggtctccaagtcgctt

SEQ ID NO: 213 - RNA, 2381 nt, synthetic construct
  source 1..2381: mol_type = other RNA
atggaagtcgacaccgagaatccagacccacgcgcgaagaaaagatgcactcggaagagcggtgtggaacaatcgctacacgcagaaggcgatgaagagtcaagctcaacaagacagtcgtgtatatgtctggatctcgagaggactctggtgaaatctgtgatgccaaagtacattcgagaccgcctcgagagaatgtccaggtacaccgggtccttcaggtacatcgttggtctattctcaaactacgtcgtcatcaggagactcgagcagggcctgcaacctcctgaggtcaagaaagtcttctacgataggtgttgggccgccatcaacacccgcatatccggaaagaacacgagaaacgacttctccgacctactcgacgagttcatcgaccagtccggtttcgacacatctctcttccctcccagggtcccctgtcgactccaagaaaccgtcacgagagaaatggaggtatctgccaagaactctattgttgtgcacctggaggccaagatcaaggggttcctgaggtttaggctcatgaacgattcccagttggcattccaacacctccccgcgaaggatcgatcgagaattatcgtctctctgtcaaacgactgtctggaacgagagatgagaggagacctcgatcccgggtacgtaccccacgtgctacaagtgagggaccttctcgcacaagtttattcgaccgaaccggacgtatcagtgctcaaggtcttgaagaagaagccccatctgttcctcgaactgatcagcatgatctcacacgttgccgaggaggcgtccgacaacaacagggacctccgagaggaaaccaagacgagatccgacgaggaaaaagtgaggaaggccttctatatgtcagagaggatcaagcgaggactggtggatcgacccaagatgtgcacggtcctccctgtctggaagctagccccgtgctttccatactactcttcttccgtggtcgggagcatttttcacaaggagggtcacgacttctcgtccgtctcaaggtttatttccgagcacttcgaccttgggagggttaataggaaagggtacaagccgtcgggatttaggtcagacggctaccaggttcaggtgacctttatggccctggtatccaaaaagccccacgttcctggtacgaaagatctggccaagtccgggtatcagattcccaagaaggtggtctccctggaaacgcaagaaaggggcttgttcgtgctctcccaggcgagaaaggacagtcggaagattgtcgctgaccgacttcatgcatacaacctgaccgtcgtcgatccagggtgcgcttcagtggtttctgtgaggtcgtgtcccctggaattctgcaggtgtgtcgggagcgttcgcgagagaagcacggcctgggagatgaagggaaccgagtactccgcgaagtgtgggaggaccatgttggaaggaagagagaagaagcggagatcgaacgacgagtacgggcggtgtttccccagattttccgcggtcaagaagaagacagccagaaagtcgtcttttctcgcgtactgtcgtgtggcggccgaaacgttcaaggttatgttcgcggaaaagatgaagagggcgaggaagaggtcgaggttccactcatcgaggcttgtacagaagactgttgacaagctggcttcgaacatagctgcgtgcccttccgaccgcaagagcattgttttgttcgggaacggcagcttcagggcaaagaaagggcacgcctccgcgcctcgaaagaagctcgtgagggctatctgttcgagggtgaacgtggggatgttggacgagttcaggacttccaagatgtgccccggggggtgtggcggggagatgactgatgttcagggtggtcagcgtgtgcgacagtgtacaactgtttccgtgggcgttgaaaatccctgtcctcttttcgaaaacggggtggcatttaggtgtgatagggatgcgagtgccaccctcaacttttgtctcgcgggatactgtgggttggttcgcaagtcgtggccaactcatctcctgagacgtcagtaagaggacagaaagtagtccctctgggacatggccggtagattgacgcagatgtaaggccaggcctctcattcacacgagaggtctattgttggacgagtacgttggttggatcattttggtcttgttaactgtattcctccgtaatagggcggatgtagccacctgtacatattttcttgtgcgctcctctacaggagacacgtgcttgtcttgattgaagtctgttacaaggtattctgcgccgaggtaataggttattagaccagagatgacaaaggcggtggcggcgtaagcgtagc

SEQ ID NO: 214 - RNA, 2573 nt, synthetic construct
  source 1..2573: mol_type = other RNA
atggtgagaccatcttcggatggaaactcgcacaagaagaagaaacacaagaagatacctccgagggcacctccaaagaacatcccgggctttctgcagacggtggcgagcttgtacccgaatcaatacctcacggcctcaggaaatctacccaaaagttgcaagggtacttactgttgccagcttttctcgagagtcaggttcggaaaggaagtgtctcagcaacttcggacgaggaccgtgagcgtcggatttcgggcggcaacgaccgatgagaaacgcgacacatttcagtccaaggtcaatgatgcggtcgatcatctctcccgtgaacgagttctggcgtctctcttcgcaaactttgtgttcatggaacgtcttcaaaacgggtcagcccttccggagcccgacagggcttttttcaagtcgtgcttgtcatcatgcatcttaagcagaggagggggaagtctcaacgccgacttcgaccgtttttccgagataacaggtttgacaagactcgtgcctcccaaaagcatgaaccttgaccaacaacgagagcacgaggcacagcgtatggcgacggcaacgtcgacgcgtgcaacacaccacgcggaaaagaggaggatttcgatcacgaggtggtttctgcgagggcggatatctcgaggcgaccttgattgcaagaagtacgcgacgaagctctacaaattggcggagaagatcatcacaaacttcgacggcacacccgaagacacacaaaagcttcgaataatgtcacgagcgatagaccctggtacagacaccaacatcatcatcgacttcgcatggcaggagaaggagttctctgatcgagaccgtggagacggcaagagcgcagagacgatcaaacacctaaactggatgttcaacacatatgtcgccgagtccagaagaatgtacgaggaggccgtgtcccgatcccacttgttcgctcccggtaaagacaaggcgtgcagaaagaaaagggacgagttcgtgaagcacgagctgggcgaggagttcacctacgctccccccaaagacactgcggtgctacccatttgctcaacgggggcggtgttcatctacatcgacatgaagacgctcgggtcatgggggctcctgcactccaacgatgcttggtggtacaaggacgtcctccgacctttttccaaagaggcaaatgtcaagtgtctcgtgaacaaggagaacgccaggtacgcgtcaagcgagcatggctacctctcgagtctgctagaaagtggtccggacaagtgcccctggatgattggcgagagtttcttgacggacggtgtacagatcaagcttttgcttaagacactagaacacggaaggaaagctttttccgggtccagcgagctgaacgaggaggggtacaacaagcttccccgggctgacgttcagctagaaagcctcttgggagagggaagaggggtgtacaacatatccagccttttgaagtcgacaagcatcagggaagacgtcgtcgtgatgtccgctgacccgggccaggccaaggttatcaacgtttcttcggctacggctagaacttgggcgaagcaagaccctggcgtcatcctcaaaacttcgacgtgtgtgttcggggacgattatcgtcgtgatactcttgcatcacgttcagacgagtacgagactttcaggcgcaagggccagccgtatggagtagccatcgatcgtcttggcaacgagaagaaaagaacttcctgtctcgcaacgttcatccagtactgcagacggtggtgtgtaaatggtccggtgttattccgagagactttgcagacaggaaggaagttcatgaggtttgaccgtttcagggctacgcagaagacactcgccttgatagcagacaagtacatgggacacgtagaagacacttgcagggtcatgttgttcggcaaagcgtcttttcgtgcgcagaaagggagggcatctgcaccaaggaaagctttgatcagagagatggcaagtagaggcgtggtgttgatggtgaacgagtacaacacttccaagatgtgcccggggtgtggagaggacatggtcgaggacaaagaacggagaatccggagttgcaaaaactttaagatcggatcaccggaagagtcctgtagacttcaccgcctgacaccagagtttgaaatggatcgcgacgacgtagggtcgactaatatcggtatgcgtggtttcggcaaattgctcggtcaggtgtggttttagggattttttccggattttcacttgcggatgatactttatagttggttttcctaaagttgacaatggtgagcgcctccacgacatccttttgctatgcgccttccgtaacgcgctgacctccgtggccaagatgcttgctgtggaagcatcttctgtggcaggaccgttgtacgtgactcgctctttggacttgacctcgagaactgaaagcagtgcctgccagaagcaagggtcaaactgctcgattgctgggagacctcccacatctccgtccacaacaactcggtcgaacagctttt

SEQ ID NO: 215 - RNA, 2640 nt, synthetic construct
  source 1..2640: mol_type = other RNA
atgtccgcatcacttctaactgagacatcgttggaagaggaacagagaagagaggttaacaacaacaccagtaaggaaaatgatacaatggaaaaaaagactgagacggctgcaacaacagaaaaagacccagaaccgtctgtcagtaaaaggtccagaaataaagaacccaaaacaacttctactgtttacacttctgtaaagtgttacctttcttccataatcaagagtgaaagtagtagaagtaatgtcacctcaaccaaagaaaggtttgaggagaggtgtaaatccgtaagcaagatgatggtcaaaggttcactgtttttgaggttagtagtggacgagtgtctgagacgttacaaccatctagaagacgaaatcgataaatggccagatatgacgaaggataacttttacgtccaattgttgaggaagggtttagacaagaagaaattgaaagaaggatctacacatcctgttgtagaagatgtttggaattcccccatcgtccaagaaacattcctatcccagcaaggagaaggaaataatcccataaagagacatctcatggatttcaataccatcacctacgccgccaaacaactaaaaacttgcttcgaaacaaacctacgcacccatttccggacacgacaacagagggccatatctggatggttagctgaaaacgggttcgataaaaagtatacgaaactcgtacaacactggataattggatgtacctacaagagtgattgggtggacagtggtgatttggaaagggtaaaagaaggaacgaaaaatttcgtgactcttcataggaaacatttatgtgttattagtgataagaagaatggtacaatttcctattcacctgaagagaaatatccgataccctcaatactaaattattacaagtttctacaaacagagtatccacaaaacaagaaaatacagaaaatgatagttgtcccaaaacacaaactaaagatacactattgtacgtttgaccaaacgaccattcaaggaatttgtaaagatttgggagtgtggaaggatatggaagaacgacacaaacaatcagaagatatactttacaagcaaggatggtacctattattcgacgttaaaaagattaagaaattgcgtccaaactggaactttcactctatccagacggacggcgaaggcgtctctgtactattttccagagaagtggaagaagtagagactgtttccaagaaaagtaagaaaaataaaaaacctagaggagatgaggataggagaaattacccgcccactaatgccaagtacgtagtgggtgtagatccgggaagaactaatgtcgtttcctgttcggtatttgatacccgtcaaaaaagggtagtgagaaaacacagaatgactgcgaaacaatactatcaagaatcttggatgacagatagaagaaaggcaaacgaaacgtacaagaagaacaataaagagtacaaagaggcgttagaggaaataactaggtacgataatggcgaagaaattataaatgatggtaacggtgatacttctacacccactaaaaaattcgaagcttacttgaaggtagtgaacgagcactacaggttactgtggaacgaaaagggaaagaaaaagtacaggaaaaatgccatgaaagtatactctagaaaacaaaagtgcatatctaactttatagatgaattaatccctaaaagggataaaattgaagattaccacattgcttttggggatgcgaaatttgcctgcacgggaagaggtgagcaatacgcatcacctgccaggattttcgccaagaagataaaggaaagagtcggaggtgataagaggtttactttcgtggacgagaaatatacgtcaaaagtatgccatcgctgcaatcaacctttaaatatgctggagaaggattgtttttcaccgaataagaaaagaaaaccgccgacaatagtaacaaccacaacaacaacaacaacagaagaagacgaagaaaatggaaaatggaagaaggctacacctctcagagaaaatagagataccagaagatgctcgtccgaaaagacgcaattcggttacagttcaaaccgaaaagtatcgacaggagatatctctatggaaacgccagtaccttcttccacttcctcttccttttgtactcctacttccattacatgtgtcttgggaggaaaattcgtcgacagggacttcaatgcaagcaccaatattgttcataaatttctagggttttgggataaaaagttaatggaaaagaaagacaagatgccgttgaagtatcactttattcgagttgcctgagaataggatgtaaaaaactcgtgtcactagtgaaatagtggcgtagccctgaaggatgagaagtatgctattttcgaccttacggtcattgtggtatatcaactcttgcccgataaagggttattcaacaacataaaaatggtacaatattctaggtgtgttttactattctttgcttgaaatcccaaaaaggtgacccttcaagttcacttgatacactaacttcaacaaatggggatatttctgccttcaccttttgcacaacagactctatgaatttattacacatcaaattcaatt

SEQ ID NO: 216 - RNA, 2018 nt, synthetic construct
  source 1..2018: mol_type = other RNA
atgcctgtgtgcaaatcttgtggtggactagaccatcagcgtagatcgtccacgaaatgcgccaactttttgcctcaagtgagaaaacgaaaactggaaccgcaacaagtgaacttggatttgcaagtgtacgtcgtgaaacgaggtttacagagcgctttgtgtccgttgttgagcgacgacgaccgcaatagactactttacgagatcaggcgagacgtacgagatttgtctagggtctacatcgaattgggcattctgttcaactattacatgaaacggtgcgacacgttgcccgacaaaccggacgttttaaattttttctacgccatgaaaggcaaaggaccgtacgctgcgtgctacaaacatttggcgggagaccaagtgtacgacggtaaattgagaagttttgtggtgcaagaactggccaaacagtacgagacactaatccataccaatatcgagactcatgcttattcgagactagctcgatattttggagtgaaaaagaacgaccatggcttattcttagcctactacaagaaagattttagtcaagccaccgaggatatgattagagtgtgccaaattattaacgtcgacagctgcaagaaaaattggtggtctactgtcccactatggctgaagatccaacaggatttgttcgacagagataatgtagaatctttcaggctatttcctcaaccttcgtatggattaaaacacttgacatacacatctcggggatggcacgaattattgagaagagtcaaacccgactctgttcctagtcactggcctaatattcaagatcacatcaaagaactgtgggctccttggctcaaatctacaaaaaactttggctgttgtctacagacagacggtgtgagtgtcagtctttctatgaatcgttggattaagaaggtgacaaagaatggtattactaaagaggtggtaattaagaaatggaagcctagctacgacaagctaatagcggtggatcctggatcaagagttcccgttgcagcttttaactctcatggattttacaaacgtgtcaccagtaaatttgttaaatcacacagtctagattggaaaagggagagggttagaactaaattgactaaatctagtgaaacggctgaggctaaagatagagaacgagtacaaacgacttgcaatattgtgttgtcttccaagaatacaagtcacgtggtagagtatacagtgtttcgacttaaatggttcgacgtcagacagaaaccctacgaaacacactctaaactcaccagactcatgtttgataaatacataaacactgccaagttgcaagaaaagattgtaaaagaagtgtttgacggaggaggtgtggaaaaaactttagtgttgtatggtgccggatcaaagtttatgaacgcggctcaattctctggtcgaaaattcaagcacgatgctctattgaaaaagcttaggcagaaaagaaatattcatgtagaaattgtagacgaatcgtacacttctcaagcctgttccacgtgtaaccagcagactggagagtttagtaaaattgtaatggatcacaagaagcgagtgggagtttgcccaaactgcgccagccgcttcgatcgtgaccagaacgctgccaaaaacattttaattaactttatgaggtctaccgtctgtaggccaagatgctcttacactaaaagggtatcgtagcttcctgaaacgtaaatgttgtaaaatcaaacgcaataaggttcaatttcaaattggcatttatttgcgtttggttctcaggaggttatctacacgtcgacaattttgccatgacatacttggtttcatgtcggaactctcgctcacacccaaacaatgcacagcacaacagattgttacgctgctggtgtgtacgagcgccctcaaaatcataatctttgtcgtcaatcaatttacacacaaagcgcagatactgcgactcgtaaggctcgggtagattgcgagatttcataaagtcc

SEQ ID NO: 217 - RNA, 2016 nt, synthetic construct
  source 1..2016: mol_type = other RNA
atgcctctgtgtaaatcttgcggtggactagaccatcagcgtagaacttccacaaagtgcgccaattatttgcctcaagtgagaaaacgaaaactggaaccgcatcaagagaatctagagttacaaatctatgttgtaaagagaggactacaaagtgctctatgtcctctgttaagtgacgaggataggaacaagttgttgtacgagatcagacgggatgtcagggatttgtctagagtctacattgaattgggtattgtgatgaattactacatgaagcggtgtgatgtgttaccagacaaaccagacgtactcaactttttttacgccatgaaaggcaagggaccttacgctgcgtgctacaaacacttggcgggagacagagtgtacgatggtaaactcagatcgtttgtagtgcaagaactagccaaacagtatgaaacgttgattcacactaatatagagacacatgcttattcaagactggcaaaatattttggagtaaaaaagaacaacgatggcttgttccaagcatattacaagaaagattttagccatgccacggaagaaatgaaacaaatttgtcaagttattaacgcagagagctgcaagaaggattggtggaacaccatacctctatggctaaaaatccaacaggaattgtttgacagagacaaggtagagtcttttaggctattccctcaacctagctatggcttgaaacatttgacctatacttctagaggatggcacgagctactcagaagggttaaacctgacacaacacccagtagttggatgtctattgatgatcatataaaggaattatgggaaccttggctaaagtctaccaaaaactttggatgctgtcttcaaacggatgggattagtgtgagtctatccatgaatcgttgggtcaagaaagtaacaaagaacggtgaaacaaaagaagtagtgattaaaaagtggaagccgagctacgacaaactgatagcggtggatcctggatcaagaatacctgtagctgcctacaattctaacggtgtatatcaaagaattactagtaaatacgtcaagtcacacagtctagaatggaaaagagatagagtgagaaataaactgactaaatctagcgaaatggctgaagctaaagatagagagcgagtacaagcggcttgcagtattgtattgtcttcgaaaaatacaagccacgtggtagagtatacagtgtttcgtctaaaatggttcaacgtcagacaacaaccctatgaaatacactctaagctcaccagattgaaattcgacaagtatataaacactggaaaagtacaagaaaagattgttaaacaagtgtttgacggaggaggtgtggagaaaactctggtgttatatggtgcaggatcgaactttatgaacactgctcagtttagcggtcgtaaatttaaacacaacgctctgttaaagaaactaaggcagaagagaaacatacatgttgaaattgtagacgaatcgtatacttcacaagcctgctcagcgtgcaaccataaaagtggagtgtttagcaaaataagcatggatcacaagaggagaaggggagtttgcccaaactgctccagctcgttcgatcgtgaccagaatgctgccaagaatattttaatcaactatttaaggtctgccgtctgcaggccaagatgctctataagaagggtatcgtagcttcctaaaacaaaattgttgtgaaatcaaacgcaataaggttcaaattcaatttggcattatttgtgtttggttcttaggaggttatatttaccacataaaatatacaacttaaccaattggttttgattaaaatctgcactcgactcgctcatttttgcaccataacggtacaatagaaataaaattttattgacaaataagtttgctgtaaatttgttcatgtacttaaaggtggtctctatataacatggtaaataagctgaactgtatttttttaagagatatctttcaattgaa

SEQ ID NO: 218 - RNA, 2121 nt, synthetic construct
  source 1..2121: mol_type = other RNA
atgccgctgtcgtgcaagtcgtgcggcggcgaaggtcacactcgccgcagctcgaaatcgtgtgctaactatctgccaccgattcggcaacgaagaaacagcggcgacgtagaccactacacacaggactttgacgtgggtgttgtgaagcagggtctcgatggtacaatctgtccactcctacgagaagacgtgcgccaacgtctgaaggtggagatcagatccgatgttgtggagctgtctaaactttacatattgctcggtgtgttcgtaaatcactgttggaacgcataccccgacgacgaatcgaacagcgttcgcacgtttcaggacatgatgaactacgtgtatgcgatcagaggcaagggaccacacgcgaagaaattcgaagatatggtgcgctatcacggcaatatacgtcgctacgacggacgacttcgttcattcatcgttcaagaagtggctaaaacctattggactgtgctgaagacgaacatcacgacgcacgcctactcgcgcctcgcccgttacctcggtgtgaagagaaacgatccgacccttttcgcagcctactatcacaaggagttcggtgacgacgattcgatatcgagggtgtgtagattcatcaacgtatcgagcgactggcattcgaccataccgatgtggatcgaaatacagcgagacatgtatgcgcgcggcgaacaatcattcgtcatcttcccgcagccgagccatggtttgaaacacgtcacgtacaccagcaaaggatggcacgaactgttgagacgcgtcgatccatcgaacgtcaaatcgaattggccttccataacggatcacaagaaagagttgtgggcgccatacctagacacgtgtgtagttgacatgaagaagtttggatgttgcattcaaacggacggtgtagccgtgtcgatatcgatgaatagaccgaagaggacgtccgtccgtcgaaaggcagctctacccaaatcgtatacaatcgacgctttcttcgaagaccggatcgtagcggtggatcccggcagtcgagtaccggtggcggcgtgcgactctcgcaccggattcaaacgcatcaccaaacgatgggtgcgttcgcacactttggagtggaagcgcgatcgatatagatcgcgcaaattgagacgcgtcgaactgaacgaagccgctgatagacaacgcgtcgagttaaatagaggagtgcaaataacgtgtcgcaacggcgagcaagttaagctgtacaccgatttccgactaaagtggttcgatgcgcgccaacttccgttcgaacgaacgcgcaaactgactagattgtcgttcaacaagtacatgatgacggctcgcatgaacgagcgtatcgttagagaaacgtttctcagtgatacaccgtcggatcgagtgttggtgctgtacggagtgggcgtcaactttgtcaacgtggcctgctacagcggtcgcaagttcaaacatacagatttgctgcgtcgattgagagcaaaacgcaacatcagagtgcaactagtagacgaatcgtacaccagcaaagcgtgctccgagtgtaacgagctgagaaacggaacgtacactcgactgcggatgaaccatcgacttcgtcgcggcgtctgtccagactgcaacgccgacgtcgagagggactacaacgccgccaaaaatatactagttaattatcaaaggtccgccgtctgcaggccgagcccgagctgtgactcgggcgtagccaccgacacggacccgcactccatacggtccattttccaatgcgccgtatcggagcccgggtcttaggaggttaccgtgcacccttctcactcggacgtggggtgattgcgagacgtgcggccaacgcacgaatcgtgctacgacgtgtcgacgacaagtgtgccaccgttgccatcgttatcacgaacattgtcagtactgtgaacgcagtagtgagacgtcagtcgactcgtaaacatgatactgttcgttacgtgtttgttcgtcaccaccgtttacggcgattgctcgactacgacgctgtcgtttgtccgaaaaaccggaaactgcttcgattacgaacagggatcggtgagccatcccacg

SEQ ID NO: 219 - RNA, 2126 nt, synthetic construct
  source 1..2126: mol_type = other RNA
atgccaccgtcgtgtaaatcgtgcggtggtgagggtcactcacgtcgcagctcaaaatcgtgtttaaactatcttccatcgattcgacaaaggagcaactgcgatgatgtatctacggaagatttcgatgttggcgtcgtcaagcggggtctcaatggagtgctatgtccactgctggacgaagatgtgcgccgacgtctgaccgctgagatcagatcggatgtggtcgagctctctagactttacgttctactcggcgtgttcgtcaaccactgttggaacgcataccctaacaacgaatcgaacggcgttcgtacgtttcaagacatgatgaactacgtttacgcgctcaaaggcaagggaccgcacgcgcataaattcgacgatatggtgagatatcacgacgggattacgtactacaacggacgcctgcgcaattattcggttcaagaaatggctaaaacgtattggaccgtgttgaagacgaacatcacgacgcacgcctattcgcgcctcgcccgttacttcggagtcaaacgaaacgatccgaccctcttcgcagcctattatcacaaggagtttggtgacgacgatatatcgagagtttgtcgattcataaacgtatcgagcgactggcattcgacaataccgatgtggatcgaaatacaacgagagatgtatgcgcgcggcgaacagtccttcgtcatcttcccgcagccgagtcacggtttgaagcacgtcacatacaccagcagaggttggcacgagttattgagacgcgtcacaccctcgaacatcacatctaactggccctccataacggatcacaagaaagagttgtgggcgccgtacctcgacaccagcgtagtggacatgaaaaagtttggctgctgcattcagacggacggagtcgccgtttcgttgtcgatgaaccgaccgaagagaaagcctgcattacgaaagaagcctatatcgacattacctaaatcgtatgtaacggatacattcaacgaagaccgcatcgtggcggtggatcccggtagtcgtgtaccggtggcggcgtgcgactctcacaccggattcaaacgcatcaccaaacgatgggtgcgctcacacacgttggagtggaaacgggagcgatacagatcgcgcaaattgagacgtgtcgaactggacgaagccgaagatagacatcgcgtcgagttagatatgggagtgcagatcacgtgtcgtaacggccatcgagtcaagctgtacaccgatttccggctaaagtggttcgatgcgcgccaacgtccgttcgaacgaacgcgcaaactaactcgattgtcgttcgacaagtacgttatgacggctcgcaccaacgaacgcatcgtgagagagacgtttctcggcgatacaccatcggatcgggtgttggtgctgtatggagcgggagtcaactttgtcaacgtggcctgctacagcggtcgcaagttcaaacacacagatttgttacgtcgattgagatcgagacgcaacatcagagttcgactcgtcgacgaatcgtacaccagcaaagcgtgctccgactgcaacgagcttcgaaacggaacgtacacccgactgcggatgaaccatcgacttcgtcgcggcgtctgtccagactgcaacgccgacgtcgagcgggactacaacgccgcgaagaatatattagttaattttcaaaggtccgccgtctgcaggccgagcccgagctgcgactcgggcgtaaccaccgaacgtgactcgcactcgatgacgatacggatcatttttcaatgcgccgtatccgagctcgggtcttaggaggttatgtatgtgtcattaggttacaagtcattaagacaaaatgagtaacggggaaaacaaaatcgaatgggatgtaaggcgtggcgtggccctcagtcaccttcgaaagcgtaatgaaaattcgatacgtcaaccagcgttacaaaaccctaaaactcttgatatgattagtacgcttgcgttgaataacgcaagcgtactacatggtgaaatgcttcgaacacttcgacgttcaccaccattgttgcgagaaaaacttttgtcttctatatcgacagtgccgttacgagtaacgc

SEQ ID NO: 220 - RNA, 2177 nt, synthetic construct
  source 1..2177: mol_type = other RNA
atgaaaaagaagaaaaaaggtgagttccaagaatttaggaataatgaaaagtcggcatacaaaactttcaaaataccaataaaatctattttatataattacgacactacacaaccagttatcaataatttagtttttgaaattaatgatttaatgatacacgcttaccaatttattcggttatatgtgttgagttgctatactaataaacagacattacctataatagatgatatatttattttatactgcataaaaacattaggcactcgtgataatagaggaaagaaatgtaaagatacagcacttttagacacattagaaaagttttatttggaagaatatcaacctttacttaaccacgaaaaaactaatttgaaaaacactacttttttattaccatatttagcaacgcaaattcatacttccttatccaacaatgcacaagagcattttattcaacattttttgagatttattaacacaacaacaactgctataactgaagataaatccatcttattcaaattgaaacaccaactaatgagtttagataatgaaacaaatgaaatgtttaatgaatggaaaataactcatttacataatatttttccacaaaacattaaaaaatcaatacactatgatgtgaaagttagaccatttgaatatttgaaaggaatgttgtatatgaatgaagtattagaaaaggaagaacataaattatttcaacccttaccgctacgcacaaatattattccaaaacatattatattggatactgcaagtttggttagtttgttttgtcctgaaacagataaggatggaaacaaaaataaaaaggggaatttattaagtaatataaaagataatcaatatgatatttggaatagttttttgaacttgaaccataaaatatttagaaaccaacattatcaattccatcatcaaatacaaacggatggtatttcgtgttgcttgttgtttattagaaaagatttgaaagataagaaatggggttcaagagttccaagcattccagaacaagatttttacaacattgaagatttatcaaaagaacaattggatacactgaaagatagaaatatagttggttgcgacccaggaaaacgcagtcttgtttatatgatggataaaaatggtaataaactacaatatactgcaccacaaagaaaacgagaaagcaaagcaaaaacaaaccaacgaatattattagaagaaaagaaacgacataaaattattgaaaaagaaaccgagttatcattacaaaatagcaaatcagttgattatatcaagttcaaaacatatttagttgaaaagcataaacttaacaaggaaacaaccgaattttacaagaaggaaacttggagaaaaatgaattttaggcaatatagttatggtaagaaatcaatagatacatttcttaataaaatccaagaaacctttggatccaatatattaattggttatggaaattggagtagaagcacacaaatgaaacattttatgcctactatgaataaaggattaagaaaacaaatccataaaaagtatgatacaataacaatcaatgaatgtaatacaagcaaaaagtgttgcgattgtaataaagaccttgaatattacaaggataaagaaaacaagaaggtatttcgtcttttagtgtgttctaactgcgtgagttgcgaaaacaaaaaaatcgtatttagaaccagagatgctaattcctcaataaatataatgaaattaacaagttgttggatagatagccaaactcgtccaactgcatttcattctcatatttcgtctttcacctgttccactatcaaagaagaacaggaaaaagtaagaccatcgtaggtgaaattcctactattgattttacattttttcttatttttttgccctgtaaaatgggcgtttgaaatgagaaaaggtgtaaagcctctaattcaaactgtggttgagtctggaaccgactttttagacaaaaacgttcattattttgtatataacaagcagcgtgatacacagaaaattttcaatatgctgtctgcgtgccagtgttgcgagcgtcatcaagtggataaaccaagagcaatggagccgtgggttgaaaccaagtttaatatgacgcaaacaactccttgtgaatgtat

SEQ ID NO: 221 - RNA, 1862 nt, synthetic construct
  source 1..1862: mol_type = other RNA
atgaaagaggcagttaaaaatgtcaaaccaaaagttcccgctaaaaagaggattattactggaagtaaaactaagaaaaaggtttttgtcaagaagaaaccacctgataagaaacctcttaagaaacccgtcaaaaaaactgttaaaacatataaactaaaatctatttatgtttctaataaagatcttaaaatgtccaaatggattcccactcctaaaaaagaatttactgaaattgaaactaacagttggtatgaacacaggaaatttgagaatcccaatggatctcctatacaatcctataacaagattgtgcctgtagttcctccggaatctattaaacaacagaatttagcaaacaaacgtaagaaaacaaatagacctattgtttttatttcttctgagaaaatcagaatttatcccaccaaagagcaacaaaaaattttacaaacttggtttcgtttatttgcatgtatgtataatagttctattgattacattaacagtaagaaagttgttcttgaatctggtagaattaatgtcgctgcaaccaggaaagtttgtaataaaattagcgttagaaaagctctaaaaactattagagacaatttaatcaaatccactaatccatcaataatgacccatataatggacgaagcgattggtttagcttgttcaaattataagacatgtttaaccaattatattgaaggtcaaattaaaaaatttgatattaaaccttggtctatctctaaaagacgaaagattattgtcattgaacctggatactttaaaggaaattctttttgtcccacagtttttcccaaaatgaaatcttctaaaccacttattatgattgataaaactgtgactttgcaatacgattctgatactagaaaatatatcttatttgtacctagagtcactcctaagtattctgtcaataaggaaaaaaatagttgtggtattgatcctggtcttagagatttccttacggtttattctgaaaatgaaactcaatcaatttgtcctattgaaatcgtagttaacaccactaaaaatgaatacaagaaaattgataagatcaacgaaatcatcaaaactaaaccaaatcttaatagtaaacgtaaaaagaaacttaatagaggcttacgtaaatatcatagaagagttactaataagatgaaagacatgcactataaagtttctcatgaattagtaaatacttttgataaaatttgtataggtaaactaaatgtaaagtctatcttatcgaaagccaatactgttctaaaaagtgccttgaaaagaaaacttgcaactctgagtttttataggtttactcagagattaactcatatgggttataagtatggcactgaagtggttaatgtcaatgaatatttgaccacaaaaacctgctcaaactgcggcaagattaaagatcttggagctagcaagatttacgaatgtgagtcttgtggaatgtacgcagatcgtgatgagaatgctgccaagaatatcctaaaagtaggattaaagccttggtacaaacaaaaatagtctaataaaatcaggggtacattccgctagtactccaccctacgggttaagcaaatgagaatatcgaaacggtatgcacaggattcttcgagtgataatcttaggatgactcactaaggagatgactaaagtgtatcattcaatattgtattgaacggtattcttccatagagagttgatttttggagtatccagaaatatcaactttttatgagcggcgtttgtgttatcggtagtaaatatacctatgaataaatatttggttgaacatctttccattaactataaacataaaagag

SEQ ID NO: 222 - RNA, 1749 nt, synthetic construct
  source 1..1749: mol_type = other RNA
atggtttataatcaggatctctctatttctaataagacttgtggtatttccgattggttgcctcctgttagaaacaaaaagatttcagatatcaagtccaatagttggtttgacattcgtcgatgtctcaatccaaatatcaagacaaaatttgttcctgttaacaataaacagattgttttaaaagaacctaattgtatatatctaaaatatgatctcattactctgaatcctagttctgatcatattcgaattttacgtgaatggtttagtctgtatgctaaaatatacaattatgccataaatttaattaggaaaatgttttatgtcaacaacaagttaattaaatccagacttcaatgtgaaatttatgatgaaattatcattcggaaattatcaaatgataaaaccaaacttattgaatcattaaaattatctgatagacctccagaatttttgttggactatgcgatttcacatgcttgtacatactgtaattcgcaagtcaaaaaatatttaaaagactctaaaaatccatacaacaagaaaactcttagatttagattatggtctttacataagagtcgtagagtcatgcatattttacctccgaaattatgtgataaaaatgtttgtaatgggttatttttgaagctcaatcccagcaaaaacatacatgatatttatatgttttgcgtaatgagttgggatagattaactggtaaatttttcgtctataaacctgtgttaaaacgactaataaatattcctaaaaaacagtttacaagatgtggtattgatccaggtgtcagaacttttttgacactttattcatcagaaagaacttatgatgtatgcgattctacattttacaataacaagattaaaagaatgtggagaaagattgatcgtattaattttcttttacacaatcgtaaaaaacgtaagaaacgtaaaaatgatagacgtttaagaaaatcattggcaaaatattatcgtaaaatcagacaaagaatcaaagatcttcacacaaaaactgcgaaatatttggtttctaggtacaaagagatttatattgggatttatgataccagcaagaatctctcagatcctagaatttcattttatgaaaaacgcagaatagccacactcaatcatgaacttttcttgacaaaacttcaacaagtagctgatagatttgggtcttcagtatttgctattaacgaacatatgaccacaaaaacatgttcaaaatgtggtcgtattaacgaaataggttcaagtaaagtccataaatgtgcgtgtggaatgtatgcaggtcgagatgaaaatgcagctaagaatattctgaaaaaaggaattaaagaacattatttatcagctcaaagtgataaaaatgttacagaatataacaacaaaaagtctaacaatacaaacaaagaaacttagtagccttcatctcatcgtattaggattacgctctcatcctaacagtatttcggattaaccccccgatttgatagagcgaacacataattaataaacttattggtttatcaaagtcaactttaataaaccaatagcaattgatttgattgactattacgatacaaaaaaaattgaaaaaaaaacattctgatataaaaaataaatagtagtaatagattacggggggttaatccgaaatactgtacagataagactaattacaagagagattaatgagacttttttaaacaacaatcacaa

SEQ ID NO: 223 - RNA, 1881 nt, synthetic construct
  source 1..1881: mol_type = other RNA
atgttttggaacaaccaatgtgccaaattatccaaatctttatggattccttcaaacatcatcaatttaaaatcaaacaaaaaaaaatattttaaatgtgataatccaaatatttcttttaattattttactgatggtattaaaatcaataaaaaaattaaatttaacgatattaaagtaactgataatcatgacaaaaaagagaaactcagaaacaaacttattaaaacagaaaatactagatatatcaaagaacaaaataatcaaaaatgcaaattaaataaacaagagctaaatgacaaacataaaaagtttcttgataaaattgagattaaagttaatcaattggacggatttattagatcgcgaagaatacaaatactccctaataaaaaacaacaaaatattattcagaaatggatttatgatacaacatctatttataataaattagttagtagttttaatcaaatatacgacaagtgtcaaaaaattgtagacaatatgaatgttgaaaataaattttaccatttgggaaaaattatcaaacaaaatacagaatttcccattaattttcacaaattgagagcattaaaatgtagtgattttattcaggattatgacaggattccctattgtgttattgctgatattataaaagaatttgtatcaaacattaaatcctgtattacaaaaatttctaaaggactaataacagaatttaaatttaaaccaagaaaatataatagaattttttcatatattcctttagaatcacattatacaactgtcagaggattttatccatctatatttgggaaaattaaaaccaatgaaaaaatattttcatggttagatattaaacatgattataagctaatttatgataagtattcaaacaagtattatattcatgttcctaaatatgtttatcgaaatgatttgacaataaaaaagcctatagcaataatggatccaggagcacgaacttttcaaactatttatggtttagatcatgttgtcaccattggagatgatttaggatccactttcaaaaaaagattattaaaaatagataatttaaaatccaaattatccaaaccaggaaaatataagcataataaaaaactagatagaaaaaccaaagtgaaaaaatggaaatataagagagccattgatagacatcataaaaagatggatcatatacaacaagaattacattataaaacagctaattacttatgtagccattatgatcgcgtggtagttactaattttagtagtaaaaaagtcggatcaaaaaagaatgatttagatgcaataacaaaaagaatccttggaaaattatctcattatagatttagacaacgccttctaacgaaatgtgaggagtatggtagtcaatactatgaggtagatgaatcgtttacgagtaaaacatgctgtaaatgtggaaatatacacaaaacattaggatcatcaaaaatatatgattgcataaaatgtaaaaacaaaatagacagagatgtgaatgcatcaatatgtattctgataaaaaataaagatatactattaaaataaaaaaaacaaggagagcggtctgctccttaatgggaaatgtgacaaatgtataacaaatgtctataattaacatagattcatacatttacatattttttaagtgatcggcaatatctaatgtatcagttaataatatatccgttgatgtatttaaaataatattttgctgatcacaataattttgtatctttttatctctttttattgaataaggtgtataatcatcattaatgtatattgcagatatattaatgtttttatgtatttcatcgattacatctatttcatcaccatatgttg

SEQ ID NO: 224 - RNA, 1782 nt, synthetic construct
  source 1..1782: mol_type = other RNA
atggtatacattaaaaaccgtataccaatccattatccaaacaaaaattgtggactacccaattgggttccttctattaacaattattatgattgtcttaatacaaatagttggtttgatatgaatatttgtaaaaatcccaataaatcttctgttaaacgtcatcttatagatgctcccattgttcctaatgaatcttataaacaacaaatgaaatccattaaacgttgcaaatctaacaaagaacctgttttcatttataccagaaagattagaatttatcctacggatgatcaaaaatgcattttagaagaatggttttgggctgctactagaatgtataatattacagttaaatatattcgagattttttgtttaaagaccataaactcacttgtccttttgcatctaaagatatattaattaattttccttttagaactgttttaaaggaagacagggacaaacttatgaagtcaatgaaaaatcccattgtgggtcatcttttggatgaaatcatagcacaagttgtttctaactataaagcagccatttccaatatacttgttggtaatattaaaaaatttaaaatttcgacttggaaatatatcagagaaaaatatattcttaaaatagaaaaaaatttgttttcgaatggaacattttgttcaagaacgttcaaagaattaaaatcatcagaacccttaatcaatatccaacacacttgtactttacaatataacagagatattggaaaatatatcttactagttcctacccacaaagaaaccaaatcatacgtcaccaatgatttagattgcggaattgacctaggagtgaggacatttgccactgtttattctagaaacaatgtcgttactattggaaacaaattcaagaaaattgattcatgtcataagaaaatagataaaataaatgagttacttgattcagacaataataagtgtaaattaagaaaagccaaaagaaaatattatcgtcgaataaaagatattattaccgatatgcactataaatcggcttactatttggttactaattacgataatatacacattggtaaattaagtaccaaaggaattctttctaagaataacaagaaaatatcccgacatacaaaacgtatggttggtgtattaagtccatatttatttaggcaaagattaaaccatatggcacataaatatggagctaccgtttttgaagtgaatgaatatctaaccacaaaaacatgttctaattgtggaaaaataaatgaactggaagccaaaaaagttcataaatgcaaatgtggaatgaaaacagatcgagatgttaatgcagcaaaaaatattctcaaagtaggatatcagtataacagtactctgcaccaagttaacgatatagatattattgaaacagacaatcatatagttacttttaataaagtgattaaagaatatgaagttttcgaagtataaaaaatatcataggcaaattccgctagagccttaaatgaaattattaggatattgaattaaattctatgggattcaatttaaatattcttgaccaataattctattggaataattttaatggatcactttaaaaaaaattgatattttcttttaaatatcaataaatataaagttttctaaagcggatcatgtgaatttatactcattataaaataaaatgataatatattagatttttatcaatctaatatatcatacaaaaattatggattgattcgggatttaatatatcaatttttt

SEQ ID NO: 225 - RNA, 1487 nt, synthetic construct
  source 1..1487: mol_type = other RNA
atgcgttgtcatgaaatttcatggccatccgggaaactcgatcaacatctgagaaacaaattttcgatcgggaaacacaatgatatcgaacaatacatttcgattcatggaaataacatcgaacccgaacagatcatcgagacaacgaagatgaacacggaaacaaggaaaaaggaaccttcgagacgttatatcaatccgtttttcctcggaaaactctggttgtctgagtgtcccaaggattttagagcgagagcaaccttcaaagctgcagatgcctataaaacttctctgacgttgtggagaaaaggactcattcgatcattcgagatgaagttctctacgaagagacgagaactagaccgtggttattgtttcggaatcgagaagaaagtgaagttcgaagatacaggtctcaaaaaacgtgatggtaagttaataataaccggaatagatggagacatcagatttttcgaaaaacctccgatcaataaagaacccgtagcaggatgcgaactatcgaaagatagttgtggagattattatcttcatgttcctatctttcgaaagaagaaagaacggaaaggagacaacttcgtagctattgaccccggaggcgttgttccgtgggccttctatgcttcgaagggagaatcgggatgtttgggaactgagatgaatgagagaatgtcgaatgtgtcgaagaagatatcaacgattgataagaaactttctggacaacctaaaggcgataaaaattcgaacgatgttatcgagcgccgaaaactttttaggaagaagaaacgcattcgagatcatgaacattacagagtcatcaactttcttacggatcgttatgacggaatcattttaccaaaactaaaaacgaagaacatttcgaaggcgctgaaatccaaggcgaaccacgacatgttcgatatcagtcattatacttttctgagacgtgtcggagagcgttgtatcgaaagtgatacgattctcgaacatcccggagagcaatggacttcgaaaacatgtggaagatgtggaaagtcgaatcttccagggagagaacgtcgaggaactttcagagagtataaatgtctttattgcggattgcatgcgcatcgagatgtccatgcggctcgcaatattttcatgaaatggtatatcgaactgaaaaaataatataaatcatcgggttagtcaagtgctccgagaggagacaatcccttacattgtggaaaatgaaagtatttcgatacttttaattaccatttgatgtataaaatgaacggctgcagcacgatttttgaaagtaaaaaatggttcacatatttcaaaatgtataaataatcccacaaaatataaatcggcatataaatacttctggcgccggggatccagctgatattctgacagctgtgttaccgagtaaaacagggccaaagctcgttgttgaaaaaggtgattatcgcaggttcaac

SEQ ID NO: 226 - RNA, 1599 nt, synthetic construct
  source 1..1599: mol_type = other RNA
atgacattgaaaccatggtggtcgaacaaatcacaacaacttacgaaaattatatcttggaacccgacgaacacaaatatatttcaaaatgatactaccatcaaaaaagtattacgaacccgaaaagtaaaactgaaccctacgaaacaacagaaaatgatgctgatgaaatttgcggacgctgccagatactcttataacatcgcgatagctgcggtcaatgataaaacacacaaagcgaataaaatagaacttcaaaatgcgttttcttctctcaaaagaagagatgatgattcatataatcaattctttcaaaaacgcagatggttgttggcaacacctcaaccaataagaaaacaagcaatctttgaagcagcaaagaatttcaaggcagcatttacgaaattgaaaaacaaaaacatcgatcacttcaaaatgaagttcaagacgaagaaacatcaacgacaacatgggttctctctcggtattggttcacatctaaaacatgacaacggtgttctcacaattttaccaagaagtctgggacaaatgcgatatttcggtttccttccgttcgaaggcaaacccgacgctgaatgtagaattcagagagatccatatggtgatcattggttgttggtgccggtgtataagaccacgaagacgcaaatatcaaaacctatcgtcgccatcgatccaggagttcgcactccttttgcgtgttttcgatccgacggcgattcgaagtgcatcggtgaagatatgaacgccaaactcatagacataaagactcgagtgaccatagtcgatcgacggatttcgaagtgccaagatggttccctaaaacgcaaattacgggagcatcgacgagtattatttcgaaagcatcaacgcgtgagagatgcgttccattggaaaatcatcaacgatatcaccgacgaatgctctggcgtgctgttgccaccattcgaaactcaacgcgtcagcaggatgttgaaagccaaaacaaactgctcgatgcttggtatcagccactacactttcaggatgcgaatgaagggaaaatgtgaggaaaagggattgctatacgatgaaccgacagaggaatacacttcgaagacatgtggtgtttgcggcctgattaattatctgttgggcagcaagaagacattcgagtgtttctgcggcaatgtatgcgatcgcgatgttcacgcagcccgcaacatcctcctgaaatggctgagctccgaggcaggtgcccgggtcgcagagaccttccttgcctctcggtcgagttagacctcttggtgacgatacttatatcgacactgagtgcttatattattcggcgtatacggatgatatccatagatattatctgctagaataatatttgattaacttatatgttagtctttagtaaatttcttatgaaaacagaaccattcatgattcatctcgtggttctcgcgatgttcgtcacgatctatcgtttcttccctggcggattcgaaaataacttttctgttgcaaatccggacaaaaaggcatcatggatagattgtttatatttcggagtaacgacacactcgaccgtcg

SEQ ID NO: 227 - RNA, 1550 nt, synthetic construct
  source 1..1550: mol_type = other RNA
atgaatataaaaaattatcattggtttaaaaataaaaaacgaaaaaatattaatactgatggttggtttaatttaaaatctattaataataaaggatttaatgattatgacaatatagaaaaaccaataaataaatttttatgttgtaaaacaattgaaatttatcctaatttagaacaaaaaaatattattttaaagtggatggatttatttattaatatgtataatcacactaattactttataaataataatatttatgattttacaaatagaaaaattaaaagtaatgttaaagaaattttaaattttagaaaacttagagataattacattaaaaataaaaaaaatgaaacatgtaaagatgaaattaataaacatatgttagatgaagcgataaatcattgtgtttcaaaacataaaagctgtataacaaattattttagtaaaaatattaaattttttagaattagaaatatgaaaaaaaatcgtagaaaaaaaatattaattattgaaaaaggtttattttcaaaaaagaaaaatgggttttgtttaacaaaattacaagaaattaaaagttctgaatctattggtgtaaataaaacaagtattttacaatatgataaacatttaaacaaatttacattatttattccaattgagaaaaatactacaaatatagaaaatagaacaaaatgtggaatagatccaggcattagaacatttttaacttgttattcaaaaaaggaaatattagaaataggtgaaaattgttatgaaaaatataaaaataattataaaaaaatagataaaacgagagaactttttgataaaaaagaaattaataaaaaacaatttacaaaatcaataaatcataattttgataaaattagaaatttaacaaaagatttacattttaaagtttctagttatctatgtaaaaaatttaatataattacaataggaaaaatcagtaccagtagtattgtattaaatacaaataatttaaataaaatatcaaaaagatgtatgttatcattatcacattttaaatttagagaaatattagaatatcaatgtaataaatataatactcaattaaatatagtagatgaatcatatacaacaaaaacttgtagcaattgtaaggagttaaataatataggttcatcaaaaaaatatcaatgtaaaagttgtaattttaaatgtgatagagatataaatgcatcaataaatatttatttaaaataactaataattataggcatgctacttgtcttgaataaaagttaggtaattttctacgaattattactgtttttataaataaatataaacttttaagtagcgggaatttgatagaaacatgtattaatacaaataatagggacctagaaaataatattgatattatagattataataaagattttattgtgtctaattatgcaacagaattatttttagaaaaacttgataatttaaaagaatttagtttaacactagattcttttaagttattggaaagtgatcttattcatattaaaaat

SEQ ID NO: 228 - RNA, 1526 nt, synthetic construct
  source 1..1526: mol_type = other RNA
atgaaggacaagggggtggttctgagatgcaggaaggtaaggatacttccgagtgaggaacagcgagaaatactcaagtcttgtttcggtatccatcgacacatctataacgagtgtgtcaaggccgagaaggacggggacatcgatggagctggggttagggagtgctacaagtggcgaaaggctttcactacaagagaggactactatggacaggggaagaattggaaagacgattgccccagccataccaaacaacaagccgtcgaggaatttttcaaaaataaaaagacgggacttaaactagtctcggagggaaagataaggaggtttgacatcgggttcaagagccggttcaagtgtcgccaagaaaccattccgtttgaaaaattcttgatcaagaagaaagattcgacgaatccactgagcaagtccctggtttccactacgtacaaggggaaatcgatggatctaaggattatgggtaaggtgccaaaggtgttcaaggatcgagacgaccaagggttcacaaggaccgagatgaagattaccagaaccaagacggggaaatactacgcgattgtttcgttcgaggttccccagacttctcgtttccccgagatccacggtgacatgatctcgtttgatccgggatgcaagacctttcaaacgtaccattctccggacggaacatggggagagataggaaccttcgaaaaacaagaattgttgctgaggaaggcggataggttgaagtctaggctggagctcgagggaaaagataaggactcgaggtggagaagacgtacgcgaaggaggatgctaaatttgttcgagaaagtgagaaaccgaacggcagatctgcacaataaggtctgttcgtgggtggtcaacacctaccgattggtcctcgtcccggagttcaagacgagtcagatggtctcctctaagaaactctactctaagacgtgtagaaagatgatgacgtggtctcactaccgattcaggagcaagttgatcgcgatggcccagaaattcacggatgtcaaggttcgcctatgtaacgaggcatatacgacgaagcaatgtgggtcatgcggggttatcaatcgctccatgaccttgaacgaccgttcattcaactgtgtgtcgtgtggtctctcgtcttcaagggatggtcatgccgcgaggaacgtcggtcttcgctcgttacgattccttgtctaaatgatctaggcaaatgtagatacgggggctcctattgctcccttgacggatacctggaaagtcataaagaaatacattggttagtctacccggagtgaatgtagagggttttctatgggtttccaccctttaccgtattgaccataggagctcgctagcttcgtccaggacgaattgagtcgttcccgggttaaactccggaaaagagagagagagagagagagaaatggaaggggggaggacgagaaagaaggaataataataaacatacgtggagcttacggtgtgataaaacatac

SEQ ID NO: 229 - RNA, 1070 nt, synthetic construct
  source 1..1070: mol_type = other RNA
atgaataattcaaaaatggatttacaaattgaagcaataactacattactttcatcattagcaattaatgaaactaaaataataaaaatacaaaaatggtttagggggtgcattttgagattaaaacaattacctttaattatgtataaaattaaaaaatatttgaaattacaagcgtttcagttttcaacccaaaatgaagatggtagaataaatagttgtattgatgaagatgaagtaattaaattacttattgaaaagtttggtggaaaaataaaaaaacctaaaattagaatgtggtatgatattttagcatttgattatatgtatgggtgggttccaatcaatataaaaacaacaactacaataacaagcgataatactggtaatttggcaatgtgtgtatatgcttacacaaatgaaatattagatattaatagggataaatcttatgaaaatggtaagatgagtgatatacttttcaataaattaaaaaataaaaaatataacactaataacaaaaaagattactatttcatagtgttaaataaaaccgatgcgagtgatataattgttaatagtgtaaaaggattgacaatattaacaccaaatataaataatttaccatttcaagtttgctgggataaaaatagaaaatttaaatatgaaaacataaacaaaaagataaaactatttattgattgcttacaaaaaccaaaaccaagttggaaagaaacatttatgtcaaatatacgaacattagatttatagatattcacttggaatataagaattacatatttgtctatgtcctattttaaaccttccagaaaacataaaattatctttgaatgtattgctatttatgtatgatactatattatttaaattgcactttttttttggtttaagcattattaatccaccaccaaaataattaactttacctaaaaacgatacatttggtttccgtgttaaattataaatgtaaatacaatctttaccgagattagtgtttatggtagtaatatttcttggtgctccccattcaaaccaattattttcattaa

SEQ ID NO: 230 - RNA, 1680 nt, synthetic construct
  source 1..1680: mol_type = other RNA
atggttttttatagttgcgaaaaatgtggtaaacaatttagtcaaaagggacattacaataaacatatcaataaaaaacttccttgtgttaatgaaacaaaacttaaagaaattataggtgtagttgttaatgaaaaattgaatgaaattaataataataaagaaaaaattgatttatttatttcaaaagatttagaagaaactataaacacagaaataccaactatgacaacaaaacataatttagggcaatattttacaacccataatgaactcaaagaaaaggtatttgagtttatcttaaatactccatctaatattttagaaccatctatgggacaaggtgatttaattacatttattacagataaaataccaagtataacatttgatatgtatgaaattgataaaaaaattaaattattggataaaatacaaaaagataagattaattatggagattttatgacacaaacaattacaaaaacatacaaaacaatagtaggaaatccaccttatgttagaactaaaaaagggaacttatatattgattttaccgaaaaatgttataatttacttgaagataatggcgaattgatatttattgtgccgtctgattttcttaaattaacaagtgcatcaaaattattaaatgttatgatgacaaatggaacatttactcatatattccataatcataacgaaaaaatgtttgataatgcatctattgatgttattgtttttagatattgtaaaaatagtttaattgataaaaaagtattatataacgacaaattactctatattacaaacagcaacggattaattacttttggagaagaagaaaataataatagtgtcttgtttcaagaatattttgacatttatgttggtcttgttagtggaaaagaagaagtttataaaaatgaggagcttggtaatattgaagttttaaatggtgaagataaaattgataaatatatttatattgaaaactacccttgtgataatgagaaaattaataaacatttatcacatcacaaaaaagaacttattgaaagaggaatacgaaagtttaatgaaaataattggtttgaatggggagcaccaagaaatattactaccataaacactaatctcggtaaagattgtatttacatttataatttaacacggaaaccaaatgtatcgtttttaggtaaagttaattattttggtggtggattaataatgcttaaaccaaaaaaaaagtgcaatttaaataatatagtatcatacataaatagcaatacattcaaagataattttatgttttctggaaggtttaaaataggacatagacaaatatgtaattcttatattccaagtgaatatctataaatctaatgttcgtatatttgacataaatgtttctttccaacttggttttggtttttgtaagcaatcaataaatagttttatctttttgtttatgttttcatatttaaattttctatttttatcccagcaaacttgaaatggtaaattatttatatttggtgttaatattgtcaatccttttacactattaacaattatatcactcgcatcggttttatttaacactatgaaatagtaatcttttttgttattagtgttatattttttattttttaatttattgaaaagtatatcactcat

SEQ ID NO: 231 - RNA, 1515 nt, synthetic construct
  source 1..1515: mol_type = other RNA
gtgaaacgattacaggcatttaaattccagttaagaccaggtggtcaacaggagtgtgaaatgaggcgcttcgcaggcgcatgtcgtttcgttttcaatcgtgctctggcacgtcagaatgagaatcatgaggccggtaataaatacatcccttacgggaaaatggcttcctggctggttgagtggaaaaatgccactgaaacgcaatggcttaaagattctccctcacagccattgcaacagtcactgaaagaccttgagcgggcttacaaaaacttcttccggaagcgggctgcttttccccgattcaaaaagcggggacagaatgatgcattccgctacccgcagggcgttaagctcgatcaggaaaacagccgtatttttctgccgaaactgggctggatgcgctaccggaacagccgtcaggtcacgggtgttgtgaaaaatgtcactgtcagccagtcctgcggtaagtggtacatcagtattcagacagaaagtgaagtatcaactccggttcacccttcagcatcaatggtcgggctggatgctggcgtggctaaactcgccacgctgtcagatggcacagtctttgagcctgtaaacagttttcagaaaaaccagaagaagctggcgagacttcagcgacagttaagccgcaaggtcaaattcagcaacaactggcaaaagcagaaacgcaaaatacagcgactgcattcctgtatcgcaaatatccgcagggactaccttcacaaagtcacaacggccgtcagcaaaaaccacgcaatgatagtcattgaggatttgaaggtcagcaacatgtcaaagtcagcagcgggtacggtcagccagccggggcgcaatgtccgggcaaaatcaggtttaaaccgttcgatactggatcagggctggtatgaaatgcgccgccagcttgcgtacaagcagctctggcgtggcggtcaggtgcttgctgttccgccagcgtatacaagccagcgttgcgcgtactgtggtcatacagcgaaagagaaccgcctgtcacaaagtaaattcagatgccaggtatgtggatatacagcgaacgccgatgtaaatggcgctcgcaacattttagcggcggggcacgccgttcttgcctgtggagagatggtgcagtcaggccgcccgttgaagcaggaacccaccgaaatgattcaggcgacagccagaacgtagcagggatccacgtccttcagggcgtggaggatgtcaatatcggtttctgtcagcgattaaattcaacaccacgtaacaaacgcctccggcaattaatccccaaaacgcagaaccaatcccgaccagcgtcaatccacttgccgttaccagaaatgccaccaccgccgcgtctcgctcacgctcattatgcagcgcctgatacaaactgccgccgatggtacttaacagcgccagacctgccagcatctggatccaacttacgggcagggcagccatcatcccggtaatggcactaccaaa

SEQ ID NO: 232 - RNA, 1862 nt, synthetic construct
  source 1..1862: mol_type = other RNA
atgaaagaggcagttaaaaatgtcaaaccaaaagttcccgctaaaaagaggattattactggaagtaaaactaagaaaaaggtttttgtcaagaagaaaccacctgataagaaacctcttaagaaacccgtcaaaaaaactgttaaaacagataaaccaaaatctatttatgttcctaataaagatctcaaaatatccaaatggattcccactcccaagaaagagtttactgagattgaaactaatagttggtatgaacacaggaagtttgagaatcctaataaatctcctgtacaaacatacaataagattgtacctgtagtacctcctgaatctattaaacaacagaatttagcaaacaaacgtaagaaaacaaatagacctattgtttttatttcttctgagaaaatcagaatttatcccaccaaagaccagcaaaaaattttgcaaacttggtttcgtttatttgcatatatgtataactgtaccattgattacattaatagtaagaaagttgttcttgaatctggtagaattaatgtcgctgcaaccaggaaagtttgtaataaaattagcgttagaaaagctcaaaaaactattagagacaacttaatccaatccactaatccatcaataatgacccatataatagatgaagcgattggtttagcttgttcaaattataaaacatgcttaaccaattacattgaaagacatattaagaaatttgatattaaaccttggaatatgtctaaaaggaagaaaattatcattatagaggctaatttctttaaaaaaggaacgttttgtcccacagttttccctaaaatggaatcttcaaaaccactcactatgattgacaagactgtcactttgcaatatgattctgatactagaaaatatatcttatttgtacctagagtcactcctaagtattctgtcaataaggaaaaaaatagttgtggtattgatcctggtcttagagatttccttacggtttattctgaaaatgaaactcaatcaatttgtcctattgaaatcgtagttaacaccactaaaaatgaatacaagaaaattgataagatcaacgaaatcatcaaaactaaaccaaatcttaatagtaaacgtaaaaagaaacttaatagaggcttacgtaaatatcatagaagagttactaataagatgaaagacatgcactataaagtttctcatgaattagtaaatacttttgataaaatttgtataggtaaactaaatgtaaagtctatcttatcgaaagccaatactgttctaaaaagtgccttgaaaagaaaacttgcaactctgagtttttataggtttactcagagattaactcatatgggttataagtatggcactgaagtggttaatgtcaatgaatatttgaccacaaaaacctgctcaaactgcggcaagattaaagatcttggagctagcaagatttacgaatgtgagtcttgtggaatgtacgcagatcgtgatgagaatgctgccaagaatatcctaaaagtaggattaaagccttggtacaaacaaaaatagtctaataaaatcaggggtacattccgctagtactccaccctacgggttaagcaaatgagaatatcgaaacggtatgcacaggattcttcgagtgataatcttaggatgactcactaaggagatgactaaagtgtatcattcaatattgtattgaacggtattcttccatagagagttgatttttggagtatccaaaaatatcaactttttatgagcggatacattagaaacagaagagaaatactgaaatatttgcgccagaaaggatacgttatctagattttcaactaaaaggattt

SEQ ID NO: 233 - RNA, 1221 nt, synthetic construct
  source 1..1221: mol_type = other RNA
gtgaaggtatacaaaaggttgtatgaactcctgggcaggggatacaaaccgttaaagagtgcaaggttaataaaacgtggaaaagattactacatagggataaccttgcaaaaagctgtcaaggaaaagaagataaaaaaaccgaggtatgtaataaacgtggatttaaacgttcagaggaatttagcttgcatagggatatttgaagtagattgggaaaaaagagaaagcaagctatacgggataaagtttgtaaacggaaagcttttgagacttgtatacaaaagggattacttgtttgaggagataaggaagaaacagaggcagacaggaaggagtccacaggtaggagataattcaagactgtggaaaaaagtaaacaatttaaacagagacatagctttaaaagtagcgaaagaaataagcgatatagcaagggagttttcagagaaaggagaagttatagtagtatttgaaaaactaaaaggactcagaggtaggaaaggaagaagcaagaagctaaacagaaagataaacttctggatgagaaggaaaatacaggagagagtaaaggaactcggattagaagaaggatttggattagattttgtgtatccgcattatacgtcaaagaagtgtagcaaatgcggttatgagggagagaggttctctccctcgggttcaaaagcgttgtttttgtgtaagaagtgtggatatgtggtaaatgcggacgtgaatgcggtattcaatcaacactttctttacctgtcccatctcctaaatggaggtgggaaagcccgccctgtggtgcgggttgggacttcgctgaaaagtccctcacgtgagggacacaatctaagcggagtccctaaagcaacaactacctttttctatatttcttcacttttgtctacattttaaggtagctgttgcaaccactcatcaataactcttctagttttctaagcattcctttacaaccgagcggtttatccaaaataaggcaaacacccttctatcaagtttccctttataggttattcccttttcagagaaatagctttagagggaagttttcttccctaaacatacgcttccatcttcagttgtccattatcatggacaacgaaatagataaatagcttagaagataaatatttgacaaagtaagcagaaaagtagacatttagaattatgacggttaaacaagaagacaagctt

SEQ ID NO: 234 - RNA, 1884 nt, synthetic construct
  source 1..1884: mol_type = other RNA
atgaaagaggcagttaaaactgtcaaaccaaaagttcccgctaaaaagcggattgttactggtagtaaaactaagaaaaaggtttttgtcaagaagaaaccaccggcaaaaaagccacctgataagaaacctcttaagaaaactaccaaaaaaacagttaaaaaagtgataaatagacccaaatctatacatattcctaacaaagatctcaaaatattcaaatggattcccactcccaagaaagagtttaccgagattgaaactaacagttggtatgaacacaggaaatttgagaatcctaataaatctcctatacaaacatacaataagattgtacctgtagttcctccggaatccattaaacaacagaatttagcaaacaaacgtaagaaaacaaatagacctattgtttttatttcttctgagaaaatcagaatttatcccaccaaagagcaacaaaaaattttacaaacttggtttcgtttatttgcatgtatgtataatagttctattgattacattaacagtaagaaagttgttcttgaatctggtagaattaatgtcgctgcaaccaggaaagtttgtaataaaattagcgttagaaaagctctaaaaactattagagacaatttaatcaaatccactaatccatcaataatgacccatataatggacgaagcgattggtttagcttgttcaaattataagacatgtttaaccaattatattgaaggtcaaattaaaaaatttgatattaaaccttggtctatctctaaaagacgaaagattattgtcattgaacctggatactttaaaggaaattctttttgtcccacagtttttcccaaaatgaaatcttctaaaccacttattatgattgataaaactgtgactttgcaatacgattctgatactagaaagtacatcttatttgtacctagagtcactcctaagtattctgtcaataaggaaaaaaatagttgtggtattgatcctggtcttagagatttccttacggtttattctgaaaatgaaactcaatcaatttgtcctattgaaatcgtagttaacaccactaaaaatgaatacaagaaaattgataagatcaacgaaatcatcaaaactaaaccaaatcttaatagtaaacgtaaaaagaaacttaatagaggcttacgtaaatatcatagaagagttactaataagatgaaagacatgcactataaagtttctcatgaattagtaaatacttttgataaaatttgtataggtaaactaaatgtaaagtctatcttatcgaaagccaatactgttctaaaaagtgccttgaaaagaaaacttgcaactctgagtttttataggtttactcagagattaactcatatgggttataagtatggcactgaagtggttaatgtcaatgaatatttgaccacaaaaacctgctcaaactgcggcaagattaaagatcttggagctagcaagatttacgaatgtgagtcttgtggaatgtacgcagatcgtgatgagaatgctgccaagaatatcctaaaagtaggattaaagccttggtacaaacaaaaatagtctaataaaatcaggggtacattccgctagtactccaccctacgggttaagcaaatgagaatatcgaaacggtatgtataggattcttcaagtagtaatcccaggatgactcactaaggagatgactaaagtgtatcattcaatacttcattgaacggtattctttcatagagaattgatttctggagtatccagaaatatcaactttttatgagcggtatatacatcacattattagactaatgattatttgtaatttcatgaatatttgtatgatagatccgaatgatgtttataaat

SEQ ID NO: 235 - RNA, 1632 nt, synthetic construct
  source 1..1632: mol_type = other RNA
atgaagagaatgagcagtttagcctaccattttggcttgaaagtacgcttttatcctagttctaaacaaaagaaaatcatcaagctaaattatgatgctcagcgttttgtctacaactcttatgtaggacgtaatcggaacaattatcgggctaggggctacttagctgttagacaatatcaagtaatgccttttgccttttctgttcttaataattatgagactaagctagcagaagaagtagtcgccaataatgagttattggctaagccaaagaacattcgcgatgcttatagctttttacgcgttaaagaaattgacagtctcgctattgccaatgcgattcaaaactatcagaaagcttggaacaactatcgcaagcttggtcatggaattccgacttttcacaaaaaacgtagtgattggtcatatcagaccaactgtcagtatcctaaacagacagaagcctttcttgataacgggacagctagatttattgatgctaagcacgttaaattgcctaagctaggcgttgtccgcattgccagctttagaaaattgattaaagagcgttttcttaatcatattccaactagaattggcacagtcacgattaaaaagaccgctgacgaccagttttacttatcaatgcaactaggcagcgatattgcctttgttaaggaattgcctaagacgcagagtcaaattggcatcgacctcaatcttgataatttcttaacagcatctaacggagcaatggtggctaatccacgattttatcgcaaaaccaaaaagaagcttgcccatgcccaacgtgtcttgtctcgcaggcagcgcagagctaagaaagagggacgcaatttgcgcttagccaagaattatcaaaaacagcgtctagcagtcgctaaactgcacgataagattcgcagacagcgaaatgacttcttgcaagtactctcaactgcactaatcaaaaaccacgatttagtagttgccgaggaattaagaagcaagaacctgttaaagaatcatgccttgtctcaagctatttctgatgttggctggcgcagttttctgaacatgctggcatataaggcagacctatatggcaaagagttcaaaacaattgatcctaagtacaccacccaaagatgccatcaatgtggcagtattatgggacaaaacggttataagaaattaacgcttaaggatcgagaatggacttgtccaatctgtcaaacgcatcatatccgtgattggaatgcggcagtaaacatcttagaaaaaggcttaggcaactggcagaattctaaaataaaaaaagcagcctaagctaggctgcagtccgctagcaactcgcggactcaaaaggctttggtaattagacgacctctatcggcaatagtcaaaagattattgttggtaagtcaagtccgtatctaagcaagttggtgttctggttagcgcaagctaacgagccagccaccaagccccagaatttattctggggtagttgacttatcttgagtctatttacgggaagctattcaattcggcagattaatccgcttgaaaaagtgaagttcaagtatgtgcaatggctatctttggctattgggctggtctgtttaa

SEQ ID NO: 236 - RNA, 1092 nt, synthetic construct
  source 1..1092: mol_type = other RNA
atgaaatcttccatggctatgataaaacataaaaaggagctggttgaatatgcacggcacctgaaactaccgaacttggcagaacatgtgggtgtgatacttcatgaagcacaggagaagcaactgacctattcggagttcctggcgaattgtcttgccagggaaatccagggcagagaaaggaaaagctacctgacacggttgaaactctccggactccccgccaaatacgatctggacctgtacgactatgacagggcggaaggaatggataacaggaggctgcgcgaactgcgtgagctggtatgggtgaaccaggcatacaatcttctcctggtaggcccttccggaaccggcaagacctttatagccgccggactcgtgtatgaagccgtaaaagcgggatacgaagcatatctgatgacactggaggagctgctcacatgcctgaaaacgaaagaggtctccgcacatgcgatgaaaacatacaagcggatcatgaaggcccggctgctggccatagacgatgccacactgttccctttgaaaagagaagaagccgtgttgctgttcaaactggtcaatgacttccaggaaaggacatcactcatcatcacggcaaacaaggcactcacccgttggctggaaacattggaggatgaagcggtcacagccgccttgcttgacaggctgctctactgctgcgagattatcaggctcggaggaacaagctatcgcatgcaaaacaggaaaacaatttttagcaaccaaaacacggatataggcacgtaaaaagagttaaggaaagtgaagcatcttcgatgctggagtgggatatactaaattacctaaaaaagtggcgcacaaatttgcgcgccacaataatggcaatttattaatcaatagatggactgaatcacctatgacagtatctcattttaatgatctactaataacttgtaaaggtactattggtgaaatggcttttaatagaatcggtagtattcacatagctcgccaaataatggcattaactggcattgttggtgacttggattatattagatggttcctttatgccaatgtatcatc

SEQ ID NO: 237 - RNA, 2192 nt, synthetic construct
  source 1..2192: mol_type = other RNA
atgaatgcttctagtaaaaggaaaattattagtcagagtgagattagcaaaaaaatagctgtaatgaatgaagaaatgcaggggttttgggctaataatagttgggatataagaaaatgtccacatccttctgccatagaattaagtaagaatcctgctttaaggaatcgttgggttcgttttgaacgtgttaaaaatctgtggttaagaacagaattgaaatatttttatttttaccatttaaacaatggaatatggaatgcaaaaactgtctggattagaaaaggaacagtaattaataaaatgttagatttcttagatttaaagtatcctagcattacttcaattactgaagttcctattgaaaaagcaatgacggagtatagaacttatttgacaaaacggggtgttagaattaccactactaattataagattactgctaatcaagaaaaaacacctgtaaaagctaattcctactatgttactaatctaaaacaatttatggagttttatgagaacttttattttgatggagaggagtgggataaagacgtttgggatagacgtaacttacctttgccagatgataaggttaacccaacacaatatgaatatacaattaactttaaagggtttcggaatacatattttaaacaacttgtaaaaagatattgtaagttgagattgaacgtggatagcttttcctatgtaagtgatattgcccaaagacttaaagagttctttaattttctggacatgaaatttaaacaagttcagagagtacaccaattaacgagagtggaaattgaagcatatttaagtgaactaaacatgatgggaataaaacctagtacaataactgggaggatctctatattggaaggactatttagtacccttcttaggctagaatgggatgatgttccttccaaaatattaatttattctgaggactatccgaaaataccaagagcaaaaccacgctttatagatgaattcgtcctagagcaattgaacagtcatcttgataaattacccgaatatatagctacgatgactatgattgttcaagaatgtggaatgaggataagtgaattgtgcaccttgaaaaaaggctgtctattagaggacaaagatggagatttctttttaaagtattatcaatggaaaatgaaaaaggagcatatagttccaatatctaaagaggtagctttacttattaaagttcgggaagataaagtttcagaggaatttccagatagtgaatacctctttccaagaaaagatggatcgccattaaaacaagaaacatttagaggtgagttaaataaattagcttatgagcaaaatatagtggataaatcaggtgagatttatagattccatgcccatgcctttcgccatacagtaggaacaagaatgattaacaacgggatgccccagcatattgtgcagaaatttttggggcatgaaagcccagaaatgacaagcagatacgctcatatctttgatgaaactctaaaaaatgaatttactaaatttcaggaaaaactggttaccaataatggagatgtgcttgatctagatgaagataatgaagtcgatgatgtagagcttcaatggttcaagaaaaatataaatgcacaagtgcttccaaatggttattgtagattgccagtagtagcaggtggttgtccacatgcgaatgcatgcttagattgcactcacttctgtaccagtaagcaattcttaccacagcacgaagaacagttagagcgtacagaagagttattagccatagctaaggataaacaatggcaaagacaagtagagactaatagccgtgttaaagagcgtttagaacaaatcattggaagtttgacggggtaattatcaatggataaacaagttagaaatacaacagaaattgtacgtttggcgaagcagaaatcaaaaaagacaagggaaaaagtagacaaagcgatttctaaattttcgattgaaggtaaagttattaattttaattcaatagcaaaggaagctaatgtttctaaatcatggctttataaggaacacgatattaggcaaagaatcgaatcccttcgtgagcgtcaaataacagcaaatgtagtctcaaaacccaagaaaagttctcgttcggaggaaatccttattaaaaccttaaaaag

SEQ ID NO: 238 - RNA, 1629 nt, synthetic construct
  source 1..1629: mol_type = other RNA
atgaaaaggatgagtagtttagcctaccactttggcattaagctgcgcttttatcctagctctaagcaaaagaagctaatcaagctgaactacgatgctcagcgttttgtttataattcttatgtaggacgcaatcggagcagctatcatgccaaacgctattttctcgtaaaacaaagcaagacaatgccgtttgctttttctattctcaatcgctatgaaacaaagctggcagaaacagtagtcgctaataatgagctattggctaagcctaagaatattcgagatgcttatagtttcttacgcatcaaagaaattgacagtctggcgctggccaatgccattcagaactatcataaggcttggaataattatcgcaaaatcgggcatggaatcccgactttccacaagaaacgcagcgattggacttatcaaaccaactgccaatacattaagcaggaagaagcttatttggacaacgggacagctaaatttattgacgctaagcacgttaaattgcctaagctgggtgttgtccgcattgctggcttgagaaagctaattaaagagcgtttgtttaagcacattccaactagaatcggaacagtgacgattaaaaagaccgctgaagatcaattctatttgtcaatgcagtcaggcagcgatttagcttttgttaaagagctgcccaaaacacagaatcaaatcggcattgatcttaatttagacaacttcttgaccaattctaatggtgccatggtcgctaatccgcacttctaccgccaagctgaaaagaagctcgcccacgcccaacgagtactctctcgcagacaaagacgtgctaagaaagcaaggcgcaatttgcggacggcaaagaactatcaaaagcagcgcctaatagtcgctaaactgcacgacaggattcgcagacagcgtgaagacttcttgcaggtcctctcaactgcattaatcaaaaaccacgatttagtagttgccgaggagctaagaagcaagaacctgttaaagaatcatgccttgtcccaagcgatctctgatgttggctggcgcaagtttcttaacatgcttgcctataaaggcaggctatacggtaaagagttcattactattgaccctaagtacacgactcaacgctgtcatgcttgtggcacaatcatgggacataatggctataagaaattaaaactaaaagaccgtgaatggacatgtccaatttgtcatacttaccatattcgtgattggaatgcggcggtaaacatccttcaaaaaggattaggcagttggcagaatcctaaaaaacaagccacttaaaaaggttggaagtccgttagcaactcgtggactaaaaaggctttggtaattaggcgacctctatcactaatagtcataagaatattagtgataagcccagcccgtatctaagcaagttggtgttctggttagcgcaagctaacgagccaccaagccccagaatttattctggggtagttgacttgaacaatttggtacaggaattcaaagaattcgtgcagcgtatgctgaaagtaaaattcagccccagtttttaatttttgaaaattcaatcaagattgttttaccggtaattaaaaa

SEQ ID NO: 239 - RNA, 1566 nt, synthetic construct
  source 1..1566: mol_type = other RNA
ttgcttaaagcaataaagtttagaatttatcccaccatagagcaaaaaaccttgatacacaagcactttggctgtgctagggtggtctataactactttttagcataccgccaaaagcaatacgcacaaggcattagagaaaattactttagcatgcaaaaggcgctcactactctcaaaaaacaagaggcttacgcttacctaagtgaatgcaactctcaaagcttgcaaatggcactaagacagctgacaacagcctttgataggtttttctctaagctggcagattatcctagattcaaatctaaaaagcattctaagcagtctttctgtgtcccgcaacacttagagatggatttaggcaacaaccaagtcaagctacccaaattcaaagaagctattaaagccaagtttcataggcatttgcctacaaactctatcgtcaaacaggggtttatctcttgtgtagcagataaatactatctctctataagctatgaggataatgagcctgaacctaaacccacaactatcagaaaagctgtgggtttagatatgggtttagagtctttagtcatcgccagcaatggcgtgttctacccatataaaaagtttttccagaatctacaaaccaaactcactaaagcgcaaaggagattgtctaagaaactaaaaggttctaataatagaaaaaaacaagctaagaaagtggcacaaatccacgcttctatcaggaatagcagagaggactacctacataaaatcagtaatgagataaccaatcaatacgatttgatagcagtagaaaccttgaaagttaggaatttggtcaaaaaccacaaactcgctaagagcattgccaatgccagctggtctaggctcattagtctattagaatataaggctggttggaagggtaaaacccttatcaaaattgaccaatacttccctagctctcaaatctgctcgacctgtgggagcaacacaggtaaaaagccactgcctattagaaattttatctacccttgttgtcaaacataccaccacagagacctaaacgctagcatcaatatcagaaactatgctttgggaatgctagatgagcgacacgctatcaaggtagataaaactagggtagggattacccgaagttacgcttgtggagattccgctaacggggctgtaaccaagtatggctacatactggatactgctagttatggatcgttgaagcaagaagcccacccgtctttagcgggtgggtgattcactaccctagttaatgatgtaagaaatatttggcaagattatgccaatagcattggcaaagatataaataccgccattcaagaggcttttacaaatctaagcaaaaagaaacaagacttcactacatggctttttgacttcaagggagagagtgataagaagtttcaatacattatggatttggctaaccgcgagaaagaaagaatcttaagaaatctcaattatggggaggaggtgaacatggataattactctgtggtgttttctttggtgtgatctttgtcttactcctcttaa

SEQ ID NO: 240 - RNA, 1227 nt, synthetic construct
  source 1..1227: mol_type = other RNA
atgataaggaataaggctttcgtggtcaggctgtacccaaatgcggctcagactgaactgattaaccgcacgctgggtagcgcaaggttcgtctacaaccacttccttgcccgtcgcattgcggcctacaaggaaagcgggaagggactgacctacgggcaaacgagtagcgaactgacccttctgaagcaggctgaagaaacctcctggctctcggaagtagataagtttgctttgcagaactcgctgaaaaaccttgagaccgcgtacaagaacttctttcggactgtgaagcagtccggtaaaaaggtaggattcccacgtttcagaaagaagcgcacgggagagtcctaccggactcaattcaccaacaacaacatccaaattggggaaggtaggctcaaacttcctaagctgggatgggtgaaaaccaagggccagcaggatattcaagggaagattctgaatgtcactgtgcgccgtattcacgaaggccattacgaagcgtccgttctctgtgaagtcgagattccctacctgcctgcggctcccaagtttgcagcgggtgtggatgtcggcatcaaggattttgccatcgtgaccgatggcgtgaggtttaagcatgaacagaatccgaaatattaccgctccaccctgaaaagacttcgtaaagctcagcaaaccctgtccagacggaagaagggcagcgcacgttacgggaaagcgaaaaccaagctggctcggattcacaagcgcattgtcaataagcgtcaggatttccttcacaagctcaccacctccctggtgcgtgagtacgaaatcatcggaaccgaacaccttaaacccgacaacatgcggaaaaatcgccgccttgcactgagcatcagtgatgcgggctggggtgagttcatccggcagttggaatacaaggcagcgtggtacgggcgactggtatctaaagtcagcccctactttccatctagccagttgtgtcatgactgcggattcaagaatcccgaagtgaagaatcttgccgtccgtacatggacttgcccgaactgtggggaaacccatgaccgagacgagaacgctgcgctgaacattcggcgtgaagcgttggtggctgcgggaatctcagacaccttaaacgctcatggaggctatgtcagacctgcttcggcgggcaatggtctgcgaagtgagaatcacgcgactttagtcgtgtga

SEQ ID NO: 241 - RNA, 1374 nt, synthetic construct
  source 1..1374: mol_type = other RNA
atgccaatttttgccgccaagaaaagacaagctacactgaagaagaagcttgttctccagcagggtgatgaattgaagaaggagagtctcaacatcccgagctgggcgccatttaagctgattgatcacggacaggccgcgaatgaccaggtgcttattcctcagaagccggaggttccgggtgacgcgcttaaggatcattggactccaaagaccgtaaggatatatcccactagcaaacagaaagagatactacacttatggcttacggctacaagacatatgtacaaccttgctgtggccgagagaattcgtttgggaaaggaggccaaggaaaacgggacaaaagtcaagatgaagctcaagcacctacgcgaagttatgcccttaaagaagaactttgattacgagcgcgacccgaagctcacctatttgaagaacgtaccgtttctaatcaaggacaatggcctacgtgatttcgacaaggactatcggaccaacctggccaaatctaaaaacacgctgagacccttcaagatccactttaagaaaataaaggataagcagagcatgacagtggagaaaaaagtatggggcctcagccgtggcatataccacgacgtattccggaaaggtgcatttaggagtaccgagccactgccagaacaactcccacacgattcaagactaatacgcgagcatggaaaatggtttttagtcatcccatatgagacaagcggcctcatgccgaagaaagaggctatcaatgcagttgccgtggatcctggcgtcagaacattcctaactacctacgacagcagccaaagggtgctagagatcggtgaggatgggtatctgaagcttcagcacctggaccgcagattgcgatcccttcagtcagagatccacaagcttcgtagcaagggaattgtgctatccaaggaagaccgaaaaaggaaaatacgtaaccgcaggactgtaatcagcaggctttatcagcggaagaaaaacctggctaacgatgttcacagaaaggcatcccatttattagcgagcaaatacaattacattctgattccggtactcaatttccataacttcaaaaaaacaaaccgcagagttaaggcgaatctcgcaaatatgaagcactgtctcttcgtgcagcgcctgaagaacaaagcttctcactttgaaaataccacagtaatcgaagtggatgaggaatttacgacaaagacgtgctccttgtgtggtgtccgcagtaatatcggtgcgtccaaagtgtttaagtgcgataattgtgacgcgactttcgatagagaccacaatgcggccaaaaatatccttttgaagtaccttggcgagatagattag

SEQ ID NO: 242 - RNA, 1461 nt, synthetic construct
  source 1..1461: mol_type = other RNA
atgaccacctgtactcaatatcaggctaagaagagaaaaagatcaaatagtaagacaaccaaaccatattggaatgaggatgcggctcatatttcccagcaatggggcttgaatacgattctaccaacggcggctaaaaatcttagtcagaaaatcacatctgactcgtggtttagcgtggttaaacatccacaggtgccggttatcaatgactcattgaacttgccgcttagcagtggtgtcaaagaagagaataccagggctcgcaaaattaaggtctatccgaccaattcccagagagaaacgctcaaaaagtggtttaggtgccagcgttacatatacaatagagcgctgaagctgcaccgcgatggcatggcaatgaccataaaaggtctacgccagactttgcttaacagagatactattattctagaacctcaggagcagtggcttaacgactacagttacgatttgaaagacgaagccctttgtgatttggtcaagaactacagttataatatggcaaagtataagcagactgggatgcctttgaagttgaggttcaagtcaaagaagacaccgattcaaactctttcagtgttggaaaagcactggaacaagagaacgaagtcgttttggagcgaagtattctcgctctcgaattttcgtggtacagagccgctcccagaggaattgccaggagattcgagggttcaaaggacaaggaacaacaaattctacttggtagttccacttgctggtgaggaaatcgctagaaaaagtcccgcagaaaagtttatatttattgatcccagggtgagaactttcctaacaggatatgactctaagcagactgtggtcgagatggggaaggacgcggttgtacgcatcgagaagcttaagcgccgacgacggcaattccagtcaaaacttgccaagctcaggaagcacaagaagaggcagaaccatcggaaagccttacataggctcgacgaaaagattcagcatattgttcgtgatatgcataagaaaacagcctcttatttgtgtaaaaactacgaaaacatctttctccccaaattgaattaccatcaatgcactagactgacccgaaagacaaggtccagcatggcaaccattgctcattgttgttttcacgatcgcttgacgatgaaggcagagcagttccatagtactagtgttcatgaagttgaggaggattacacgtcgaagacttgttctagttgtggcaatttaaaagatgatctaggatctaataaaatctacagctgttcaagttgctcctcggttttcgatagagacttcaacgcagcaaagaacatcatgctcaagtatatctgcgagcatttgatggcaagtggtggatcttccattacctctgcttcaagtggaggaaatggtggattcgccatgatgggtcctggccccttcgtacgttag

SEQ ID NO: 243 - RNA, 780 nt, synthetic construct
  source 1..780: mol_type = other RNA
atgatcgtcccggcaacggtgctcgccgccgcagttgggaagaggaaacgaggcaccgggtcgtctcgtaaccagcagcggcaggcgaagcgtgtggcccgtcgagaacggaaccaggctgccgcgaagaagcgcaagacgtcgtcgggggctgtcaccacgaagcgcacgccgaagacaacaccgcgcaagcgcaagaccccggtgaagcccaagccgacgtggacgttcacgactgtcaagatgaagctgcggacgttctgcaaggacgagggtgtagcgctcgcttgggactcggtgctgagggacatgaacaagatggtggcggaggcttacgtgctggccgccgtccacgtggttcgctgctgtgagcaaggtttcgacatccccatcttggcgcgttccttctaccagcagtgcttgtcggccgtgagtggggggaacctgcacgatcgacggacgaccatgactgaagagttccgcgactcggtggcgatctacagcgcgtggcaaggcgagagcctgatccccaacagggacgccatcaatcgtggttgggcacaggtggcagccgagcgcatggcccatgaaacccagaaccatctccagctcaacttttaccggcgcttcaaacaatacattacgctccgctacgggctggctggccgcgatcgctaccttgtgctcaagggtatcctcgaccccgagtacgacggagacaacgagatcgtgctggagtacagagaattgatcccccgcaatgatgaaggagcgagctag

SEQ ID NO: 244 - RNA, 2082 nt, synthetic construct
  source 1..2082: mol_type = other RNA
atggaagtcgacaccgagaatccagacccacgcgcgaagaaaagatgcactcggaagagcggtgtggaacaatcgctacacgcagaaggcgatgaagagtcaagctcaacaagacagtcgtgtatatgtctggatctcgagaggactctggtgaaatctgtgatgccaaagtacattcgagaccgcctcgagagaatgtccaggtacaccgggtccttcaggtacatcgttggtctattctcaaactacgtcgtcatcaggagactcgagcagggcctgcaacctcctgaggtcaagaaagtcttctacgataggtgttgggccgccatcaacacccgcatatccggaaagaacacgagaaacgacttctccgacctactcgacgagttcatcgaccagtccggtttcgacacatctctcttccctcccagggtcccctgtcgactccaagaaaccgtcacgagagaaatggaggtatctgccaagaactctattgttgtgcacctggaggccaagatcaaggggttcctgaggtttaggctcatgaacgattcccagttggcattccaacacctccccgcgaaggatcgatcgagaattatcgtctctctgtcaaacgactgtctggaacgagagatgagaggagacctcgatcccgggtacgtaccccacgtgctacaagtgagggaccttctcgcacaagtttattcgaccgaaccggacgtatcagtgctcaaggtcttgaagaagaagccccatctgttcctcgaactgatcagcatgatctcacacgttgccgaggaggcgtccgacaacaacagggacctccgagaggaaaccaagacgagatccgacgaggaaaaagtgaggaaggccttctatatgtcagagaggatcaagcgaggactggtggatcgacccaagatgtgcacggtcctccctgtctggaagctagccccgtgctttccatactactcttcttccgtggtcgggagcatttttcacaaggagggtcacgacttctcgtccgtctcaaggtttatttccgagcacttcgaccttgggagggttaataggaaagggtacaagccgtcgggatttaggtcagacggctaccaggttcaggtgacctttatggccctggtatccaaaaagccccacgttcctggtacgaaagatctggccaagtccgggtatcagattcccaagaaggtggtctccctggaaacgcaagaaaggggcttgttcgtgctctcccaggcgagaaaggacagtcggaagattgtcgctgaccgacttcatgcatacaacctgaccgtcgtcgatccagggtgcgcttcagtggtttctgtgaggtcgtgtcccctggaattctgcaggtgtgtcgggagcgttcgcgagagaagcacggcctgggagatgaagggaaccgagtactccgcgaagtgtgggaggaccatgttggaaggaagagagaagaagcggagatcgaacgacgagtacgggcggtgtttccccagattttccgcggtcaagaagaagacagccagaaagtcgtcttttctcgcgtactgtcgtgtggcggccgaaacgttcaaggttatgttcgcggaaaagatgaagagggcgaggaagaggtcgaggttccactcatcgaggcttgtacagaagactgttgacaagctggcttcgaacatagctgcgtgcccttccgaccgcaagagcattgttttgttcgggaacggcagcttcagggcaaagaaagggcacgcctccgcgcctcgaaagaagctcgtgagggctatctgttcgagggtgaacgtggggatgttggacgagttcaggacttccaagatgtgccccggggggtgtggcggggagatgactgatgttcagggtggtcagcgtgtgcgacagtgtacaactgtttccgtgggcgttgaaaatccctgtcctcttttcgaaaacggggtggcatttaggtgtgatagggatgcgagtgccaccctcaacttttgtctcgcgggatactgtgggttggttcgcaagtcgtggccaactcatctcctgagacgtcagtaa

SEQ ID NO: 245 - RNA, 2274 nt, synthetic construct
  source 1..2274: mol_type = other RNA
atggtgagaccatcttcggatggaaactcgcacaagaagaagaaacacaagaagatacctccgagggcacctccaaagaacatcccgggctttctgcagacggtggcgagcttgtacccgaatcaatacctcacggcctcaggaaatctacccaaaagttgcaagggtacttactgttgccagcttttctcgagagtcaggttcggaaaggaagtgtctcagcaacttcggacgaggaccgtgagcgtcggatttcgggcggcaacgaccgatgagaaacgcgacacatttcagtccaaggtcaatgatgcggtcgatcatctctcccgtgaacgagttctggcgtctctcttcgcaaactttgtgttcatggaacgtcttcaaaacgggtcagcccttccggagcccgacagggcttttttcaagtcgtgcttgtcatcatgcatcttaagcagaggagggggaagtctcaacgccgacttcgaccgtttttccgagataacaggtttgacaagactcgtgcctcccaaaagcatgaaccttgaccaacaacgagagcacgaggcacagcgtatggcgacggcaacgtcgacgcgtgcaacacaccacgcggaaaagaggaggatttcgatcacgaggtggtttctgcgagggcggatatctcgaggcgaccttgattgcaagaagtacgcgacgaagctctacaaattggcggagaagatcatcacaaacttcgacggcacacccgaagacacacaaaagcttcgaataatgtcacgagcgatagaccctggtacagacaccaacatcatcatcgacttcgcatggcaggagaaggagttctctgatcgagaccgtggagacggcaagagcgcagagacgatcaaacacctaaactggatgttcaacacatatgtcgccgagtccagaagaatgtacgaggaggccgtgtcccgatcccacttgttcgctcccggtaaagacaaggcgtgcagaaagaaaagggacgagttcgtgaagcacgagctgggcgaggagttcacctacgctccccccaaagacactgcggtgctacccatttgctcaacgggggcggtgttcatctacatcgacatgaagacgctcgggtcatgggggctcctgcactccaacgatgcttggtggtacaaggacgtcctccgacctttttccaaagaggcaaatgtcaagtgtctcgtgaacaaggagaacgccaggtacgcgtcaagcgagcatggctacctctcgagtctgctagaaagtggtccggacaagtgcccctggatgattggcgagagtttcttgacggacggtgtacagatcaagcttttgcttaagacactagaacacggaaggaaagctttttccgggtccagcgagctgaacgaggaggggtacaacaagcttccccgggctgacgttcagctagaaagcctcttgggagagggaagaggggtgtacaacatatccagccttttgaagtcgacaagcatcagggaagacgtcgtcgtgatgtccgctgacccgggccaggccaaggttatcaacgtttcttcggctacggctagaacttgggcgaagcaagaccctggcgtcatcctcaaaacttcgacgtgtgtgttcggggacgattatcgtcgtgatactcttgcatcacgttcagacgagtacgagactttcaggcgcaagggccagccgtatggagtagccatcgatcgtcttggcaacgagaagaaaagaacttcctgtctcgcaacgttcatccagtactgcagacggtggtgtgtaaatggtccggtgttattccgagagactttgcagacaggaaggaagttcatgaggtttgaccgtttcagggctacgcagaagacactcgccttgatagcagacaagtacatgggacacgtagaagacacttgcagggtcatgttgttcggcaaagcgtcttttcgtgcgcagaaagggagggcatctgcaccaaggaaagctttgatcagagagatggcaagtagaggcgtggtgttgatggtgaacgagtacaacacttccaagatgtgcccggggtgtggagaggacatggtcgaggacaaagaacggagaatccggagttgcaaaaactttaagatcggatcaccggaagagtcctgtagacttcaccgcctgacaccagagtttgaaatggatcgcgacgacgtagggtcgactaatatcggtatgcgtggtttcggcaaattgctcggtcaggtgtggttttag

SEQ ID NO: 246 - RNA, 2340 nt, synthetic construct
  source 1..2340: mol_type = other RNA
atgtccgcatcacttctaactgagacatcgttggaagaggaacagagaagagaggttaacaacaacaccagtaaggaaaatgatacaatggaaaaaaagactgagacggctgcaacaacagaaaaagacccagaaccgtctgtcagtaaaaggtccagaaataaagaacccaaaacaacttctactgtttacacttctgtaaagtgttacctttcttccataatcaagagtgaaagtagtagaagtaatgtcacctcaaccaaagaaaggtttgaggagaggtgtaaatccgtaagcaagatgatggtcaaaggttcactgtttttgaggttagtagtggacgagtgtctgagacgttacaaccatctagaagacgaaatcgataaatggccagatatgacgaaggataacttttacgtccaattgttgaggaagggtttagacaagaagaaattgaaagaaggatctacacatcctgttgtagaagatgtttggaattcccccatcgtccaagaaacattcctatcccagcaaggagaaggaaataatcccataaagagacatctcatggatttcaataccatcacctacgccgccaaacaactaaaaacttgcttcgaaacaaacctacgcacccatttccggacacgacaacagagggccatatctggatggttagctgaaaacgggttcgataaaaagtatacgaaactcgtacaacactggataattggatgtacctacaagagtgattgggtggacagtggtgatttggaaagggtaaaagaaggaacgaaaaatttcgtgactcttcataggaaacatttatgtgttattagtgataagaagaatggtacaatttcctattcacctgaagagaaatatccgataccctcaatactaaattattacaagtttctacaaacagagtatccacaaaacaagaaaatacagaaaatgatagttgtcccaaaacacaaactaaagatacactattgtacgtttgaccaaacgaccattcaaggaatttgtaaagatttgggagtgtggaaggatatggaagaacgacacaaacaatcagaagatatactttacaagcaaggatggtacctattattcgacgttaaaaagattaagaaattgcgtccaaactggaactttcactctatccagacggacggcgaaggcgtctctgtactattttccagagaagtggaagaagtagagactgtttccaagaaaagtaagaaaaataaaaaacctagaggagatgaggataggagaaattacccgcccactaatgccaagtacgtagtgggtgtagatccgggaagaactaatgtcgtttcctgttcggtatttgatacccgtcaaaaaagggtagtgagaaaacacagaatgactgcgaaacaatactatcaagaatcttggatgacagatagaagaaaggcaaacgaaacgtacaagaagaacaataaagagtacaaagaggcgttagaggaaataactaggtacgataatggcgaagaaattataaatgatggtaacggtgatacttctacacccactaaaaaattcgaagcttacttgaaggtagtgaacgagcactacaggttactgtggaacgaaaagggaaagaaaaagtacaggaaaaatgccatgaaagtatactctagaaaacaaaagtgcatatctaactttatagatgaattaatccctaaaagggataaaattgaagattaccacattgcttttggggatgcgaaatttgcctgcacgggaagaggtgagcaatacgcatcacctgccaggattttcgccaagaagataaaggaaagagtcggaggtgataagaggtttactttcgtggacgagaaatatacgtcaaaagtatgccatcgctgcaatcaacctttaaatatgctggagaaggattgtttttcaccgaataagaaaagaaaaccgccgacaatagtaacaaccacaacaacaacaacaacagaagaagacgaagaaaatggaaaatggaagaaggctacacctctcagagaaaatagagataccagaagatgctcgtccgaaaagacgcaattcggttacagttcaaaccgaaaagtatcgacaggagatatctctatggaaacgccagtaccttcttccacttcctcttccttttgtactcctacttccattacatgtgtcttgggaggaaaattcgtcgacagggacttcaatgcaagcaccaatattgttcataaatttctagggttttgggataaaaagttaatggaaaagaaagacaagatgccgttgaagtatcactttattcgagttgcctga

SEQ ID NO: 247 - RNA, 1719 nt, synthetic construct
  source 1..1719: mol_type = other RNA
atgcctgtgtgcaaatcttgtggtggactagaccatcagcgtagatcgtccacgaaatgcgccaactttttgcctcaagtgagaaaacgaaaactggaaccgcaacaagtgaacttggatttgcaagtgtacgtcgtgaaacgaggtttacagagcgctttgtgtccgttgttgagcgacgacgaccgcaatagactactttacgagatcaggcgagacgtacgagatttgtctagggtctacatcgaattgggcattctgttcaactattacatgaaacggtgcgacacgttgcccgacaaaccggacgttttaaattttttctacgccatgaaaggcaaaggaccgtacgctgcgtgctacaaacatttggcgggagaccaagtgtacgacggtaaattgagaagttttgtggtgcaagaactggccaaacagtacgagacactaatccataccaatatcgagactcatgcttattcgagactagctcgatattttggagtgaaaaagaacgaccatggcttattcttagcctactacaagaaagattttagtcaagccaccgaggatatgattagagtgtgccaaattattaacgtcgacagctgcaagaaaaattggtggtctactgtcccactatggctgaagatccaacaggatttgttcgacagagataatgtagaatctttcaggctatttcctcaaccttcgtatggattaaaacacttgacatacacatctcggggatggcacgaattattgagaagagtcaaacccgactctgttcctagtcactggcctaatattcaagatcacatcaaagaactgtgggctccttggctcaaatctacaaaaaactttggctgttgtctacagacagacggtgtgagtgtcagtctttctatgaatcgttggattaagaaggtgacaaagaatggtattactaaagaggtggtaattaagaaatggaagcctagctacgacaagctaatagcggtggatcctggatcaagagttcccgttgcagcttttaactctcatggattttacaaacgtgtcaccagtaaatttgttaaatcacacagtctagattggaaaagggagagggttagaactaaattgactaaatctagtgaaacggctgaggctaaagatagagaacgagtacaaacgacttgcaatattgtgttgtcttccaagaatacaagtcacgtggtagagtatacagtgtttcgacttaaatggttcgacgtcagacagaaaccctacgaaacacactctaaactcaccagactcatgtttgataaatacataaacactgccaagttgcaagaaaagattgtaaaagaagtgtttgacggaggaggtgtggaaaaaactttagtgttgtatggtgccggatcaaagtttatgaacgcggctcaattctctggtcgaaaattcaagcacgatgctctattgaaaaagcttaggcagaaaagaaatattcatgtagaaattgtagacgaatcgtacacttctcaagcctgttccacgtgtaaccagcagactggagagtttagtaaaattgtaatggatcacaagaagcgagtgggagtttgcccaaactgcgccagccgcttcgatcgtgaccagaacgctgccaaaaacattttaattaactttatgaggtctaccgtctgtaggccaagatgctcttacactaaaagggtatcgtag

SEQ ID NO: 248 - RNA, 1716 nt, synthetic construct
  source 1..1716: mol_type = other RNA
atgcctctgtgtaaatcttgcggtggactagaccatcagcgtagaacttccacaaagtgcgccaattatttgcctcaagtgagaaaacgaaaactggaaccgcatcaagagaatctagagttacaaatctatgttgtaaagagaggactacaaagtgctctatgtcctctgttaagtgacgaggataggaacaagttgttgtacgagatcagacgggatgtcagggatttgtctagagtctacattgaattgggtattgtgatgaattactacatgaagcggtgtgatgtgttaccagacaaaccagacgtactcaactttttttacgccatgaaaggcaagggaccttacgctgcgtgctacaaacacttggcgggagacagagtgtacgatggtaaactcagatcgtttgtagtgcaagaactagccaaacagtatgaaacgttgattcacactaatatagagacacatgcttattcaagactggcaaaatattttggagtaaaaaagaacaacgatggcttgttccaagcatattacaagaaagattttagccatgccacggaagaaatgaaacaaatttgtcaagttattaacgcagagagctgcaagaaggattggtggaacaccatacctctatggctaaaaatccaacaggaattgtttgacagagacaaggtagagtcttttaggctattccctcaacctagctatggcttgaaacatttgacctatacttctagaggatggcacgagctactcagaagggttaaacctgacacaacacccagtagttggatgtctattgatgatcatataaaggaattatgggaaccttggctaaagtctaccaaaaactttggatgctgtcttcaaacggatgggattagtgtgagtctatccatgaatcgttgggtcaagaaagtaacaaagaacggtgaaacaaaagaagtagtgattaaaaagtggaagccgagctacgacaaactgatagcggtggatcctggatcaagaatacctgtagctgcctacaattctaacggtgtatatcaaagaattactagtaaatacgtcaagtcacacagtctagaatggaaaagagatagagtgagaaataaactgactaaatctagcgaaatggctgaagctaaagatagagagcgagtacaagcggcttgcagtattgtattgtcttcgaaaaatacaagccacgtggtagagtatacagtgtttcgtctaaaatggttcaacgtcagacaacaaccctatgaaatacactctaagctcaccagattgaaattcgacaagtatataaacactggaaaagtacaagaaaagattgttaaacaagtgtttgacggaggaggtgtggagaaaactctggtgttatatggtgcaggatcgaactttatgaacactgctcagtttagcggtcgtaaatttaaacacaacgctctgttaaagaaactaaggcagaagagaaacatacatgttgaaattgtagacgaatcgtatacttcacaagcctgctcagcgtgcaaccataaaagtggagtgtttagcaaaataagcatggatcacaagaggagaaggggagtttgcccaaactgctccagctcgttcgatcgtgaccagaatgctgccaagaatattttaatcaactatttaaggtctgccgtctgcaggccaagatgctctataagaagggtatcgtag

SEQ ID NO: 249 - RNA, 1821 nt, synthetic construct
  source 1..1821: mol_type = other RNA
atgccgctgtcgtgcaagtcgtgcggcggcgaaggtcacactcgccgcagctcgaaatcgtgtgctaactatctgccaccgattcggcaacgaagaaacagcggcgacgtagaccactacacacaggactttgacgtgggtgttgtgaagcagggtctcgatggtacaatctgtccactcctacgagaagacgtgcgccaacgtctgaaggtggagatcagatccgatgttgtggagctgtctaaactttacatattgctcggtgtgttcgtaaatcactgttggaacgcataccccgacgacgaatcgaacagcgttcgcacgtttcaggacatgatgaactacgtgtatgcgatcagaggcaagggaccacacgcgaagaaattcgaagatatggtgcgctatcacggcaatatacgtcgctacgacggacgacttcgttcattcatcgttcaagaagtggctaaaacctattggactgtgctgaagacgaacatcacgacgcacgcctactcgcgcctcgcccgttacctcggtgtgaagagaaacgatccgacccttttcgcagcctactatcacaaggagttcggtgacgacgattcgatatcgagggtgtgtagattcatcaacgtatcgagcgactggcattcgaccataccgatgtggatcgaaatacagcgagacatgtatgcgcgcggcgaacaatcattcgtcatcttcccgcagccgagccatggtttgaaacacgtcacgtacaccagcaaaggatggcacgaactgttgagacgcgtcgatccatcgaacgtcaaatcgaattggccttccataacggatcacaagaaagagttgtgggcgccatacctagacacgtgtgtagttgacatgaagaagtttggatgttgcattcaaacggacggtgtagccgtgtcgatatcgatgaatagaccgaagaggacgtccgtccgtcgaaaggcagctctacccaaatcgtatacaatcgacgctttcttcgaagaccggatcgtagcggtggatcccggcagtcgagtaccggtggcggcgtgcgactctcgcaccggattcaaacgcatcaccaaacgatgggtgcgttcgcacactttggagtggaagcgcgatcgatatagatcgcgcaaattgagacgcgtcgaactgaacgaagccgctgatagacaacgcgtcgagttaaatagaggagtgcaaataacgtgtcgcaacggcgagcaagttaagctgtacaccgatttccgactaaagtggttcgatgcgcgccaacttccgttcgaacgaacgcgcaaactgactagattgtcgttcaacaagtacatgatgacggctcgcatgaacgagcgtatcgttagagaaacgtttctcagtgatacaccgtcggatcgagtgttggtgctgtacggagtgggcgtcaactttgtcaacgtggcctgctacagcggtcgcaagttcaaacatacagatttgctgcgtcgattgagagcaaaacgcaacatcagagtgcaactagtagacgaatcgtacaccagcaaagcgtgctccgagtgtaacgagctgagaaacggaacgtacactcgactgcggatgaaccatcgacttcgtcgcggcgtctgtccagactgcaacgccgacgtcgagagggactacaacgccgccaaaaatatactagttaattatcaaaggtccgccgtctgcaggccgagcccgagctgtgactcgggcgtagccaccgacacggacccgcactccatacggtccattttccaatgcgccgtatcggagcccgggtcttag

SEQ ID NO: 250 - RNA, 1827 nt, synthetic construct
  source 1..1827: mol_type = other RNA
atgccaccgtcgtgtaaatcgtgcggtggtgagggtcactcacgtcgcagctcaaaatcgtgtttaaactatcttccatcgattcgacaaaggagcaactgcgatgatgtatctacggaagatttcgatgttggcgtcgtcaagcggggtctcaatggagtgctatgtccactgctggacgaagatgtgcgccgacgtctgaccgctgagatcagatcggatgtggtcgagctctctagactttacgttctactcggcgtgttcgtcaaccactgttggaacgcataccctaacaacgaatcgaacggcgttcgtacgtttcaagacatgatgaactacgtttacgcgctcaaaggcaagggaccgcacgcgcataaattcgacgatatggtgagatatcacgacgggattacgtactacaacggacgcctgcgcaattattcggttcaagaaatggctaaaacgtattggaccgtgttgaagacgaacatcacgacgcacgcctattcgcgcctcgcccgttacttcggagtcaaacgaaacgatccgaccctcttcgcagcctattatcacaaggagtttggtgacgacgatatatcgagagtttgtcgattcataaacgtatcgagcgactggcattcgacaataccgatgtggatcgaaatacaacgagagatgtatgcgcgcggcgaacagtccttcgtcatcttcccgcagccgagtcacggtttgaagcacgtcacatacaccagcagaggttggcacgagttattgagacgcgtcacaccctcgaacatcacatctaactggccctccataacggatcacaagaaagagttgtgggcgccgtacctcgacaccagcgtagtggacatgaaaaagtttggctgctgcattcagacggacggagtcgccgtttcgttgtcgatgaaccgaccgaagagaaagcctgcattacgaaagaagcctatatcgacattacctaaatcgtatgtaacggatacattcaacgaagaccgcatcgtggcggtggatcccggtagtcgtgtaccggtggcggcgtgcgactctcacaccggattcaaacgcatcaccaaacgatgggtgcgctcacacacgttggagtggaaacgggagcgatacagatcgcgcaaattgagacgtgtcgaactggacgaagccgaagatagacatcgcgtcgagttagatatgggagtgcagatcacgtgtcgtaacggccatcgagtcaagctgtacaccgatttccggctaaagtggttcgatgcgcgccaacgtccgttcgaacgaacgcgcaaactaactcgattgtcgttcgacaagtacgttatgacggctcgcaccaacgaacgcatcgtgagagagacgtttctcggcgatacaccatcggatcgggtgttggtgctgtatggagcgggagtcaactttgtcaacgtggcctgctacagcggtcgcaagttcaaacacacagatttgttacgtcgattgagatcgagacgcaacatcagagttcgactcgtcgacgaatcgtacaccagcaaagcgtgctccgactgcaacgagcttcgaaacggaacgtacacccgactgcggatgaaccatcgacttcgtcgcggcgtctgtccagactgcaacgccgacgtcgagcgggactacaacgccgcgaagaatatattagttaattttcaaaggtccgccgtctgcaggccgagcccgagctgcgactcgggcgtaaccaccgaacgtgactcgcactcgatgacgatacggatcatttttcaatgcgccgtatccgagctcgggtcttag

SEQ ID NO: 251 - RNA, 1878 nt, synthetic construct
  source 1..1878: mol_type = other RNA
atgaaaaagaagaaaaaaggtgagttccaagaatttaggaataatgaaaagtcggcatacaaaactttcaaaataccaataaaatctattttatataattacgacactacacaaccagttatcaataatttagtttttgaaattaatgatttaatgatacacgcttaccaatttattcggttatatgtgttgagttgctatactaataaacagacattacctataatagatgatatatttattttatactgcataaaaacattaggcactcgtgataatagaggaaagaaatgtaaagatacagcacttttagacacattagaaaagttttatttggaagaatatcaacctttacttaaccacgaaaaaactaatttgaaaaacactacttttttattaccatatttagcaacgcaaattcatacttccttatccaacaatgcacaagagcattttattcaacattttttgagatttattaacacaacaacaactgctataactgaagataaatccatcttattcaaattgaaacaccaactaatgagtttagataatgaaacaaatgaaatgtttaatgaatggaaaataactcatttacataatatttttccacaaaacattaaaaaatcaatacactatgatgtgaaagttagaccatttgaatatttgaaaggaatgttgtatatgaatgaagtattagaaaaggaagaacataaattatttcaacccttaccgctacgcacaaatattattccaaaacatattatattggatactgcaagtttggttagtttgttttgtcctgaaacagataaggatggaaacaaaaataaaaaggggaatttattaagtaatataaaagataatcaatatgatatttggaatagttttttgaacttgaaccataaaatatttagaaaccaacattatcaattccatcatcaaatacaaacggatggtatttcgtgttgcttgttgtttattagaaaagatttgaaagataagaaatggggttcaagagttccaagcattccagaacaagatttttacaacattgaagatttatcaaaagaacaattggatacactgaaagatagaaatatagttggttgcgacccaggaaaacgcagtcttgtttatatgatggataaaaatggtaataaactacaatatactgcaccacaaagaaaacgagaaagcaaagcaaaaacaaaccaacgaatattattagaagaaaagaaacgacataaaattattgaaaaagaaaccgagttatcattacaaaatagcaaatcagttgattatatcaagttcaaaacatatttagttgaaaagcataaacttaacaaggaaacaaccgaattttacaagaaggaaacttggagaaaaatgaattttaggcaatatagttatggtaagaaatcaatagatacatttcttaataaaatccaagaaacctttggatccaatatattaattggttatggaaattggagtagaagcacacaaatgaaacattttatgcctactatgaataaaggattaagaaaacaaatccataaaaagtatgatacaataacaatcaatgaatgtaatacaagcaaaaagtgttgcgattgtaataaagaccttgaatattacaaggataaagaaaacaagaaggtatttcgtcttttagtgtgttctaactgcgtgagttgcgaaaacaaaaaaatcgtatttagaaccagagatgctaattcctcaataaatataatgaaattaacaagttgttggatagatagccaaactcgtccaactgcatttcattctcatatttcgtctttcacctgttccactatcaaagaagaacaggaaaaagtaagaccatcgtag

SEQ ID NO: 252 - RNA, 1563 nt, synthetic construct
  source 1..1563: mol_type = other RNA
atgaaagaggcagttaaaaatgtcaaaccaaaagttcccgctaaaaagaggattattactggaagtaaaactaagaaaaaggtttttgtcaagaagaaaccacctgataagaaacctcttaagaaacccgtcaaaaaaactgttaaaacatataaactaaaatctatttatgtttctaataaagatcttaaaatgtccaaatggattcccactcctaaaaaagaatttactgaaattgaaactaacagttggtatgaacacaggaaatttgagaatcccaatggatctcctatacaatcctataacaagattgtgcctgtagttcctccggaatctattaaacaacagaatttagcaaacaaacgtaagaaaacaaatagacctattgtttttatttcttctgagaaaatcagaatttatcccaccaaagagcaacaaaaaattttacaaacttggtttcgtttatttgcatgtatgtataatagttctattgattacattaacagtaagaaagttgttcttgaatctggtagaattaatgtcgctgcaaccaggaaagtttgtaataaaattagcgttagaaaagctctaaaaactattagagacaatttaatcaaatccactaatccatcaataatgacccatataatggacgaagcgattggtttagcttgttcaaattataagacatgtttaaccaattatattgaaggtcaaattaaaaaatttgatattaaaccttggtctatctctaaaagacgaaagattattgtcattgaacctggatactttaaaggaaattctttttgtcccacagtttttcccaaaatgaaatcttctaaaccacttattatgattgataaaactgtgactttgcaatacgattctgatactagaaaatatatcttatttgtacctagagtcactcctaagtattctgtcaataaggaaaaaaatagttgtggtattgatcctggtcttagagatttccttacggtttattctgaaaatgaaactcaatcaatttgtcctattgaaatcgtagttaacaccactaaaaatgaatacaagaaaattgataagatcaacgaaatcatcaaaactaaaccaaatcttaatagtaaacgtaaaaagaaacttaatagaggcttacgtaaatatcatagaagagttactaataagatgaaagacatgcactataaagtttctcatgaattagtaaatacttttgataaaatttgtataggtaaactaaatgtaaagtctatcttatcgaaagccaatactgttctaaaaagtgccttgaaaagaaaacttgcaactctgagtttttataggtttactcagagattaactcatatgggttataagtatggcactgaagtggttaatgtcaatgaatatttgaccacaaaaacctgctcaaactgcggcaagattaaagatcttggagctagcaagatttacgaatgtgagtcttgtggaatgtacgcagatcgtgatgagaatgctgccaagaatatcctaaaagtaggattaaagccttggtacaaacaaaaatag

SEQ ID NO: 253 - RNA, 1449 nt, synthetic construct
  source 1..1449: mol_type = other RNA
atggtttataatcaggatctctctatttctaataagacttgtggtatttccgattggttgcctcctgttagaaacaaaaagatttcagatatcaagtccaatagttggtttgacattcgtcgatgtctcaatccaaatatcaagacaaaatttgttcctgttaacaataaacagattgttttaaaagaacctaattgtatatatctaaaatatgatctcattactctgaatcctagttctgatcatattcgaattttacgtgaatggtttagtctgtatgctaaaatatacaattatgccataaatttaattaggaaaatgttttatgtcaacaacaagttaattaaatccagacttcaatgtgaaatttatgatgaaattatcattcggaaattatcaaatgataaaaccaaacttattgaatcattaaaattatctgatagacctccagaatttttgttggactatgcgatttcacatgcttgtacatactgtaattcgcaagtcaaaaaatatttaaaagactctaaaaatccatacaacaagaaaactcttagatttagattatggtctttacataagagtcgtagagtcatgcatattttacctccgaaattatgtgataaaaatgtttgtaatgggttatttttgaagctcaatcccagcaaaaacatacatgatatttatatgttttgcgtaatgagttgggatagattaactggtaaatttttcgtctataaacctgtgttaaaacgactaataaatattcctaaaaaacagtttacaagatgtggtattgatccaggtgtcagaacttttttgacactttattcatcagaaagaacttatgatgtatgcgattctacattttacaataacaagattaaaagaatgtggagaaagattgatcgtattaattttcttttacacaatcgtaaaaaacgtaagaaacgtaaaaatgatagacgtttaagaaaatcattggcaaaatattatcgtaaaatcagacaaagaatcaaagatcttcacacaaaaactgcgaaatatttggtttctaggtacaaagagatttatattgggatttatgataccagcaagaatctctcagatcctagaatttcattttatgaaaaacgcagaatagccacactcaatcatgaacttttcttgacaaaacttcaacaagtagctgatagatttgggtcttcagtatttgctattaacgaacatatgaccacaaaaacatgttcaaaatgtggtcgtattaacgaaataggttcaagtaaagtccataaatgtgcgtgtggaatgtatgcaggtcgagatgaaaatgcagctaagaatattctgaaaaaaggaattaaagaacattatttatcagctcaaagtgataaaaatgttacagaatataacaacaaaaagtctaacaatacaaacaaagaaacttag

SEQ ID NO: 254 - RNA, 1581 nt, synthetic construct
  source 1..1581: mol_type = other RNA
atgttttggaacaaccaatgtgccaaattatccaaatctttatggattccttcaaacatcatcaatttaaaatcaaacaaaaaaaaatattttaaatgtgataatccaaatatttcttttaattattttactgatggtattaaaatcaataaaaaaattaaatttaacgatattaaagtaactgataatcatgacaaaaaagagaaactcagaaacaaacttattaaaacagaaaatactagatatatcaaagaacaaaataatcaaaaatgcaaattaaataaacaagagctaaatgacaaacataaaaagtttcttgataaaattgagattaaagttaatcaattggacggatttattagatcgcgaagaatacaaatactccctaataaaaaacaacaaaatattattcagaaatggatttatgatacaacatctatttataataaattagttagtagttttaatcaaatatacgacaagtgtcaaaaaattgtagacaatatgaatgttgaaaataaattttaccatttgggaaaaattatcaaacaaaatacagaatttcccattaattttcacaaattgagagcattaaaatgtagtgattttattcaggattatgacaggattccctattgtgttattgctgatattataaaagaatttgtatcaaacattaaatcctgtattacaaaaatttctaaaggactaataacagaatttaaatttaaaccaagaaaatataatagaattttttcatatattcctttagaatcacattatacaactgtcagaggattttatccatctatatttgggaaaattaaaaccaatgaaaaaatattttcatggttagatattaaacatgattataagctaatttatgataagtattcaaacaagtattatattcatgttcctaaatatgtttatcgaaatgatttgacaataaaaaagcctatagcaataatggatccaggagcacgaacttttcaaactatttatggtttagatcatgttgtcaccattggagatgatttaggatccactttcaaaaaaagattattaaaaatagataatttaaaatccaaattatccaaaccaggaaaatataagcataataaaaaactagatagaaaaaccaaagtgaaaaaatggaaatataagagagccattgatagacatcataaaaagatggatcatatacaacaagaattacattataaaacagctaattacttatgtagccattatgatcgcgtggtagttactaattttagtagtaaaaaagtcggatcaaaaaagaatgatttagatgcaataacaaaaagaatccttggaaaattatctcattatagatttagacaacgccttctaacgaaatgtgaggagtatggtagtcaatactatgaggtagatgaatcgtttacgagtaaaacatgctgtaaatgtggaaatatacacaaaacattaggatcatcaaaaatatatgattgcataaaatgtaaaaacaaaatagacagagatgtgaatgcatcaatatgtattctgataaaaaataaagatatactattaaaataa

SEQ ID NO: 255 - RNA, 1482 nt, synthetic construct
  source 1..1482: mol_type = other RNA
atggtatacattaaaaaccgtataccaatccattatccaaacaaaaattgtggactacccaattgggttccttctattaacaattattatgattgtcttaatacaaatagttggtttgatatgaatatttgtaaaaatcccaataaatcttctgttaaacgtcatcttatagatgctcccattgttcctaatgaatcttataaacaacaaatgaaatccattaaacgttgcaaatctaacaaagaacctgttttcatttataccagaaagattagaatttatcctacggatgatcaaaaatgcattttagaagaatggttttgggctgctactagaatgtataatattacagttaaatatattcgagattttttgtttaaagaccataaactcacttgtccttttgcatctaaagatatattaattaattttccttttagaactgttttaaaggaagacagggacaaacttatgaagtcaatgaaaaatcccattgtgggtcatcttttggatgaaatcatagcacaagttgtttctaactataaagcagccatttccaatatacttgttggtaatattaaaaaatttaaaatttcgacttggaaatatatcagagaaaaatatattcttaaaatagaaaaaaatttgttttcgaatggaacattttgttcaagaacgttcaaagaattaaaatcatcagaacccttaatcaatatccaacacacttgtactttacaatataacagagatattggaaaatatatcttactagttcctacccacaaagaaaccaaatcatacgtcaccaatgatttagattgcggaattgacctaggagtgaggacatttgccactgtttattctagaaacaatgtcgttactattggaaacaaattcaagaaaattgattcatgtcataagaaaatagataaaataaatgagttacttgattcagacaataataagtgtaaattaagaaaagccaaaagaaaatattatcgtcgaataaaagatattattaccgatatgcactataaatcggcttactatttggttactaattacgataatatacacattggtaaattaagtaccaaaggaattctttctaagaataacaagaaaatatcccgacatacaaaacgtatggttggtgtattaagtccatatttatttaggcaaagattaaaccatatggcacataaatatggagctaccgtttttgaagtgaatgaatatctaaccacaaaaacatgttctaattgtggaaaaataaatgaactggaagccaaaaaagttcataaatgcaaatgtggaatgaaaacagatcgagatgttaatgcagcaaaaaatattctcaaagtaggatatcagtataacagtactctgcaccaagttaacgatatagatattattgaaacagacaatcatatagttacttttaataaagtgattaaagaatatgaagttttcgaagtataa

SEQ ID NO: 256 - RNA, 1188 nt, synthetic construct
  source 1..1188: mol_type = other RNA
atgcgttgtcatgaaatttcatggccatccgggaaactcgatcaacatctgagaaacaaattttcgatcgggaaacacaatgatatcgaacaatacatttcgattcatggaaataacatcgaacccgaacagatcatcgagacaacgaagatgaacacggaaacaaggaaaaaggaaccttcgagacgttatatcaatccgtttttcctcggaaaactctggttgtctgagtgtcccaaggattttagagcgagagcaaccttcaaagctgcagatgcctataaaacttctctgacgttgtggagaaaaggactcattcgatcattcgagatgaagttctctacgaagagacgagaactagaccgtggttattgtttcggaatcgagaagaaagtgaagttcgaagatacaggtctcaaaaaacgtgatggtaagttaataataaccggaatagatggagacatcagatttttcgaaaaacctccgatcaataaagaacccgtagcaggatgcgaactatcgaaagatagttgtggagattattatcttcatgttcctatctttcgaaagaagaaagaacggaaaggagacaacttcgtagctattgaccccggaggcgttgttccgtgggccttctatgcttcgaagggagaatcgggatgtttgggaactgagatgaatgagagaatgtcgaatgtgtcgaagaagatatcaacgattgataagaaactttctggacaacctaaaggcgataaaaattcgaacgatgttatcgagcgccgaaaactttttaggaagaagaaacgcattcgagatcatgaacattacagagtcatcaactttcttacggatcgttatgacggaatcattttaccaaaactaaaaacgaagaacatttcgaaggcgctgaaatccaaggcgaaccacgacatgttcgatatcagtcattatacttttctgagacgtgtcggagagcgttgtatcgaaagtgatacgattctcgaacatcccggagagcaatggacttcgaaaacatgtggaagatgtggaaagtcgaatcttccagggagagaacgtcgaggaactttcagagagtataaatgtctttattgcggattgcatgcgcatcgagatgtccatgcggctcgcaatattttcatgaaatggtatatcgaactgaaaaaataa

SEQ ID NO: 257 - RNA, 1299 nt, synthetic construct
  source 1..1299: mol_type = other RNA
atgacattgaaaccatggtggtcgaacaaatcacaacaacttacgaaaattatatcttggaacccgacgaacacaaatatatttcaaaatgatactaccatcaaaaaagtattacgaacccgaaaagtaaaactgaaccctacgaaacaacagaaaatgatgctgatgaaatttgcggacgctgccagatactcttataacatcgcgatagctgcggtcaatgataaaacacacaaagcgaataaaatagaacttcaaaatgcgttttcttctctcaaaagaagagatgatgattcatataatcaattctttcaaaaacgcagatggttgttggcaacacctcaaccaataagaaaacaagcaatctttgaagcagcaaagaatttcaaggcagcatttacgaaattgaaaaacaaaaacatcgatcacttcaaaatgaagttcaagacgaagaaacatcaacgacaacatgggttctctctcggtattggttcacatctaaaacatgacaacggtgttctcacaattttaccaagaagtctgggacaaatgcgatatttcggtttccttccgttcgaaggcaaacccgacgctgaatgtagaattcagagagatccatatggtgatcattggttgttggtgccggtgtataagaccacgaagacgcaaatatcaaaacctatcgtcgccatcgatccaggagttcgcactccttttgcgtgttttcgatccgacggcgattcgaagtgcatcggtgaagatatgaacgccaaactcatagacataaagactcgagtgaccatagtcgatcgacggatttcgaagtgccaagatggttccctaaaacgcaaattacgggagcatcgacgagtattatttcgaaagcatcaacgcgtgagagatgcgttccattggaaaatcatcaacgatatcaccgacgaatgctctggcgtgctgttgccaccattcgaaactcaacgcgtcagcaggatgttgaaagccaaaacaaactgctcgatgcttggtatcagccactacactttcaggatgcgaatgaagggaaaatgtgaggaaaagggattgctatacgatgaaccgacagaggaatacacttcgaagacatgtggtgtttgcggcctgattaattatctgttgggcagcaagaagacattcgagtgtttctgcggcaatgtatgcgatcgcgatgttcacgcagcccgcaacatcctcctgaaatggctgagctccgaggcaggtgcccgggtcgcagagaccttccttgcctctcggtcgagttag

SEQ ID NO: 258 - RNA, 1251 nt, synthetic construct
  source 1..1251: mol_type = other RNA
atgaatataaaaaattatcattggtttaaaaataaaaaacgaaaaaatattaatactgatggttggtttaatttaaaatctattaataataaaggatttaatgattatgacaatatagaaaaaccaataaataaatttttatgttgtaaaacaattgaaatttatcctaatttagaacaaaaaaatattattttaaagtggatggatttatttattaatatgtataatcacactaattactttataaataataatatttatgattttacaaatagaaaaattaaaagtaatgttaaagaaattttaaattttagaaaacttagagataattacattaaaaataaaaaaaatgaaacatgtaaagatgaaattaataaacatatgttagatgaagcgataaatcattgtgtttcaaaacataaaagctgtataacaaattattttagtaaaaatattaaattttttagaattagaaatatgaaaaaaaatcgtagaaaaaaaatattaattattgaaaaaggtttattttcaaaaaagaaaaatgggttttgtttaacaaaattacaagaaattaaaagttctgaatctattggtgtaaataaaacaagtattttacaatatgataaacatttaaacaaatttacattatttattccaattgagaaaaatactacaaatatagaaaatagaacaaaatgtggaatagatccaggcattagaacatttttaacttgttattcaaaaaaggaaatattagaaataggtgaaaattgttatgaaaaatataaaaataattataaaaaaatagataaaacgagagaactttttgataaaaaagaaattaataaaaaacaatttacaaaatcaataaatcataattttgataaaattagaaatttaacaaaagatttacattttaaagtttctagttatctatgtaaaaaatttaatataattacaataggaaaaatcagtaccagtagtattgtattaaatacaaataatttaaataaaatatcaaaaagatgtatgttatcattatcacattttaaatttagagaaatattagaatatcaatgtaataaatataatactcaattaaatatagtagatgaatcatatacaacaaaaacttgtagcaattgtaaggagttaaataatataggttcatcaaaaaaatatcaatgtaaaagttgtaattttaaatgtgatagagatataaatgcatcaataaatatttatttaaaataa

SEQ ID NO: 259 - RNA, 1227 nt, synthetic construct
  source 1..1227: mol_type = other RNA
atgaaggacaagggggtggttctgagatgcaggaaggtaaggatacttccgagtgaggaacagcgagaaatactcaagtcttgtttcggtatccatcgacacatctataacgagtgtgtcaaggccgagaaggacggggacatcgatggagctggggttagggagtgctacaagtggcgaaaggctttcactacaagagaggactactatggacaggggaagaattggaaagacgattgccccagccataccaaacaacaagccgtcgaggaatttttcaaaaataaaaagacgggacttaaactagtctcggagggaaagataaggaggtttgacatcgggttcaagagccggttcaagtgtcgccaagaaaccattccgtttgaaaaattcttgatcaagaagaaagattcgacgaatccactgagcaagtccctggtttccactacgtacaaggggaaatcgatggatctaaggattatgggtaaggtgccaaaggtgttcaaggatcgagacgaccaagggttcacaaggaccgagatgaagattaccagaaccaagacggggaaatactacgcgattgtttcgttcgaggttccccagacttctcgtttccccgagatccacggtgacatgatctcgtttgatccgggatgcaagacctttcaaacgtaccattctccggacggaacatggggagagataggaaccttcgaaaaacaagaattgttgctgaggaaggcggataggttgaagtctaggctggagctcgagggaaaagataaggactcgaggtggagaagacgtacgcgaaggaggatgctaaatttgttcgagaaagtgagaaaccgaacggcagatctgcacaataaggtctgttcgtgggtggtcaacacctaccgattggtcctcgtcccggagttcaagacgagtcagatggtctcctctaagaaactctactctaagacgtgtagaaagatgatgacgtggtctcactaccgattcaggagcaagttgatcgcgatggcccagaaattcacggatgtcaaggttcgcctatgtaacgaggcatatacgacgaagcaatgtgggtcatgcggggttatcaatcgctccatgaccttgaacgaccgttcattcaactgtgtgtcgtgtggtctctcgtcttcaagggatggtcatgccgcgaggaacgtcggtcttcgctcgttacgattccttgtctaa

SEQ ID NO: 260 - RNA, 771 nt, synthetic construct
  source 1..771: mol_type = other RNA
atgaataattcaaaaatggatttacaaattgaagcaataactacattactttcatcattagcaattaatgaaactaaaataataaaaatacaaaaatggtttagggggtgcattttgagattaaaacaattacctttaattatgtataaaattaaaaaatatttgaaattacaagcgtttcagttttcaacccaaaatgaagatggtagaataaatagttgtattgatgaagatgaagtaattaaattacttattgaaaagtttggtggaaaaataaaaaaacctaaaattagaatgtggtatgatattttagcatttgattatatgtatgggtgggttccaatcaatataaaaacaacaactacaataacaagcgataatactggtaatttggcaatgtgtgtatatgcttacacaaatgaaatattagatattaatagggataaatcttatgaaaatggtaagatgagtgatatacttttcaataaattaaaaaataaaaaatataacactaataacaaaaaagattactatttcatagtgttaaataaaaccgatgcgagtgatataattgttaatagtgtaaaaggattgacaatattaacaccaaatataaataatttaccatttcaagtttgctgggataaaaatagaaaatttaaatatgaaaacataaacaaaaagataaaactatttattgattgcttacaaaaaccaaaaccaagttggaaagaaacatttatgtcaaatatacgaacattagatttatag

SEQ ID NO: 261 - RNA, 1380 nt, synthetic construct
  source 1..1380: mol_type = other RNA
atggttttttatagttgcgaaaaatgtggtaaacaatttagtcaaaagggacattacaataaacatatcaataaaaaacttccttgtgttaatgaaacaaaacttaaagaaattataggtgtagttgttaatgaaaaattgaatgaaattaataataataaagaaaaaattgatttatttatttcaaaagatttagaagaaactataaacacagaaataccaactatgacaacaaaacataatttagggcaatattttacaacccataatgaactcaaagaaaaggtatttgagtttatcttaaatactccatctaatattttagaaccatctatgggacaaggtgatttaattacatttattacagataaaataccaagtataacatttgatatgtatgaaattgataaaaaaattaaattattggataaaatacaaaaagataagattaattatggagattttatgacacaaacaattacaaaaacatacaaaacaatagtaggaaatccaccttatgttagaactaaaaaagggaacttatatattgattttaccgaaaaatgttataatttacttgaagataatggcgaattgatatttattgtgccgtctgattttcttaaattaacaagtgcatcaaaattattaaatgttatgatgacaaatggaacatttactcatatattccataatcataacgaaaaaatgtttgataatgcatctattgatgttattgtttttagatattgtaaaaatagtttaattgataaaaaagtattatataacgacaaattactctatattacaaacagcaacggattaattacttttggagaagaagaaaataataatagtgtcttgtttcaagaatattttgacatttatgttggtcttgttagtggaaaagaagaagtttataaaaatgaggagcttggtaatattgaagttttaaatggtgaagataaaattgataaatatatttatattgaaaactacccttgtgataatgagaaaattaataaacatttatcacatcacaaaaaagaacttattgaaagaggaatacgaaagtttaatgaaaataattggtttgaatggggagcaccaagaaatattactaccataaacactaatctcggtaaagattgtatttacatttataatttaacacggaaaccaaatgtatcgtttttaggtaaagttaattattttggtggtggattaataatgcttaaaccaaaaaaaaagtgcaatttaaataatatagtatcatacataaatagcaatacattcaaagataattttatgttttctggaaggtttaaaataggacatagacaaatatgtaattcttatattccaagtgaatatctataa

SEQ ID NO: 262 - RNA, 1215 nt, synthetic construct
  source 1..1215: mol_type = other RNA
gtgaaacgattacaggcatttaaattccagttaagaccaggtggtcaacaggagtgtgaaatgaggcgcttcgcaggcgcatgtcgtttcgttttcaatcgtgctctggcacgtcagaatgagaatcatgaggccggtaataaatacatcccttacgggaaaatggcttcctggctggttgagtggaaaaatgccactgaaacgcaatggcttaaagattctccctcacagccattgcaacagtcactgaaagaccttgagcgggcttacaaaaacttcttccggaagcgggctgcttttccccgattcaaaaagcggggacagaatgatgcattccgctacccgcagggcgttaagctcgatcaggaaaacagccgtatttttctgccgaaactgggctggatgcgctaccggaacagccgtcaggtcacgggtgttgtgaaaaatgtcactgtcagccagtcctgcggtaagtggtacatcagtattcagacagaaagtgaagtatcaactccggttcacccttcagcatcaatggtcgggctggatgctggcgtggctaaactcgccacgctgtcagatggcacagtctttgagcctgtaaacagttttcagaaaaaccagaagaagctggcgagacttcagcgacagttaagccgcaaggtcaaattcagcaacaactggcaaaagcagaaacgcaaaatacagcgactgcattcctgtatcgcaaatatccgcagggactaccttcacaaagtcacaacggccgtcagcaaaaaccacgcaatgatagtcattgaggatttgaaggtcagcaacatgtcaaagtcagcagcgggtacggtcagccagccggggcgcaatgtccgggcaaaatcaggtttaaaccgttcgatactggatcagggctggtatgaaatgcgccgccagcttgcgtacaagcagctctggcgtggcggtcaggtgcttgctgttccgccagcgtatacaagccagcgttgcgcgtactgtggtcatacagcgaaagagaaccgcctgtcacaaagtaaattcagatgccaggtatgtggatatacagcgaacgccgatgtaaatggcgctcgcaacattttagcggcggggcacgccgttcttgcctgtggagagatggtgcagtcaggccgcccgttgaagcaggaacccaccgaaatgattcaggcgacagccagaacgtag

SEQ ID NO: 263 - RNA, 1563 nt, synthetic construct
  source 1..1563: mol_type = other RNA
atgaaagaggcagttaaaaatgtcaaaccaaaagttcccgctaaaaagaggattattactggaagtaaaactaagaaaaaggtttttgtcaagaagaaaccacctgataagaaacctcttaagaaacccgtcaaaaaaactgttaaaacagataaaccaaaatctatttatgttcctaataaagatctcaaaatatccaaatggattcccactcccaagaaagagtttactgagattgaaactaatagttggtatgaacacaggaagtttgagaatcctaataaatctcctgtacaaacatacaataagattgtacctgtagtacctcctgaatctattaaacaacagaatttagcaaacaaacgtaagaaaacaaatagacctattgtttttatttcttctgagaaaatcagaatttatcccaccaaagaccagcaaaaaattttgcaaacttggtttcgtttatttgcatatatgtataactgtaccattgattacattaatagtaagaaagttgttcttgaatctggtagaattaatgtcgctgcaaccaggaaagtttgtaataaaattagcgttagaaaagctcaaaaaactattagagacaacttaatccaatccactaatccatcaataatgacccatataatagatgaagcgattggtttagcttgttcaaattataaaacatgcttaaccaattacattgaaagacatattaagaaatttgatattaaaccttggaatatgtctaaaaggaagaaaattatcattatagaggctaatttctttaaaaaaggaacgttttgtcccacagttttccctaaaatggaatcttcaaaaccactcactatgattgacaagactgtcactttgcaatatgattctgatactagaaaatatatcttatttgtacctagagtcactcctaagtattctgtcaataaggaaaaaaatagttgtggtattgatcctggtcttagagatttccttacggtttattctgaaaatgaaactcaatcaatttgtcctattgaaatcgtagttaacaccactaaaaatgaatacaagaaaattgataagatcaacgaaatcatcaaaactaaaccaaatcttaatagtaaacgtaaaaagaaacttaatagaggcttacgtaaatatcatagaagagttactaataagatgaaagacatgcactataaagtttctcatgaattagtaaatacttttgataaaatttgtataggtaaactaaatgtaaagtctatcttatcgaaagccaatactgttctaaaaagtgccttgaaaagaaaacttgcaactctgagtttttataggtttactcagagattaactcatatgggttataagtatggcactgaagtggttaatgtcaatgaatatttgaccacaaaaacctgctcaaactgcggcaagattaaagatcttggagctagcaagatttacgaatgtgagtcttgtggaatgtacgcagatcgtgatgagaatgctgccaagaatatcctaaaagtaggattaaagccttggtacaaacaaaaatag

SEQ ID NO: 264 - RNA, 921 nt, synthetic construct
  source 1..921: mol_type = other RNA
gtgaaggtatacaaaaggttgtatgaactcctgggcaggggatacaaaccgttaaagagtgcaaggttaataaaacgtggaaaagattactacatagggataaccttgcaaaaagctgtcaaggaaaagaagataaaaaaaccgaggtatgtaataaacgtggatttaaacgttcagaggaatttagcttgcatagggatatttgaagtagattgggaaaaaagagaaagcaagctatacgggataaagtttgtaaacggaaagcttttgagacttgtatacaaaagggattacttgtttgaggagataaggaagaaacagaggcagacaggaaggagtccacaggtaggagataattcaagactgtggaaaaaagtaaacaatttaaacagagacatagctttaaaagtagcgaaagaaataagcgatatagcaagggagttttcagagaaaggagaagttatagtagtatttgaaaaactaaaaggactcagaggtaggaaaggaagaagcaagaagctaaacagaaagataaacttctggatgagaaggaaaatacaggagagagtaaaggaactcggattagaagaaggatttggattagattttgtgtatccgcattatacgtcaaagaagtgtagcaaatgcggttatgagggagagaggttctctccctcgggttcaaaagcgttgtttttgtgtaagaagtgtggatatgtggtaaatgcggacgtgaatgcggtattcaatcaacactttctttacctgtcccatctcctaaatggaggtgggaaagcccgccctgtggtgcgggttgggacttcgctgaaaagtccctcacgtgagggacacaatctaagcggagtccctaaagcaacaactacctttttctatatttcttcacttttgtctacattttaa

SEQ ID NO: 265 - RNA, 1584 nt, synthetic construct
  source 1..1584: mol_type = other RNA
atgaaagaggcagttaaaactgtcaaaccaaaagttcccgctaaaaagcggattgttactggtagtaaaactaagaaaaaggtttttgtcaagaagaaaccaccggcaaaaaagccacctgataagaaacctcttaagaaaactaccaaaaaaacagttaaaaaagtgataaatagacccaaatctatacatattcctaacaaagatctcaaaatattcaaatggattcccactcccaagaaagagtttaccgagattgaaactaacagttggtatgaacacaggaaatttgagaatcctaataaatctcctatacaaacatacaataagattgtacctgtagttcctccggaatccattaaacaacagaatttagcaaacaaacgtaagaaaacaaatagacctattgtttttatttcttctgagaaaatcagaatttatcccaccaaagagcaacaaaaaattttacaaacttggtttcgtttatttgcatgtatgtataatagttctattgattacattaacagtaagaaagttgttcttgaatctggtagaattaatgtcgctgcaaccaggaaagtttgtaataaaattagcgttagaaaagctctaaaaactattagagacaatttaatcaaatccactaatccatcaataatgacccatataatggacgaagcgattggtttagcttgttcaaattataagacatgtttaaccaattatattgaaggtcaaattaaaaaatttgatattaaaccttggtctatctctaaaagacgaaagattattgtcattgaacctggatactttaaaggaaattctttttgtcccacagtttttcccaaaatgaaatcttctaaaccacttattatgattgataaaactgtgactttgcaatacgattctgatactagaaagtacatcttatttgtacctagagtcactcctaagtattctgtcaataaggaaaaaaatagttgtggtattgatcctggtcttagagatttccttacggtttattctgaaaatgaaactcaatcaatttgtcctattgaaatcgtagttaacaccactaaaaatgaatacaagaaaattgataagatcaacgaaatcatcaaaactaaaccaaatcttaatagtaaacgtaaaaagaaacttaatagaggcttacgtaaatatcatagaagagttactaataagatgaaagacatgcactataaagtttctcatgaattagtaaatacttttgataaaatttgtataggtaaactaaatgtaaagtctatcttatcgaaagccaatactgttctaaaaagtgccttgaaaagaaaacttgcaactctgagtttttataggtttactcagagattaactcatatgggttataagtatggcactgaagtggttaatgtcaatgaatatttgaccacaaaaacctgctcaaactgcggcaagattaaagatcttggagctagcaagatttacgaatgtgagtcttgtggaatgtacgcagatcgtgatgagaatgctgccaagaatatcctaaaagtaggattaaagccttggtacaaacaaaaatag

SEQ ID NO: 266 - RNA, 1332 nt, synthetic construct
  source 1..1332: mol_type = other RNA
atgaagagaatgagcagtttagcctaccattttggcttgaaagtacgcttttatcctagttctaaacaaaagaaaatcatcaagctaaattatgatgctcagcgttttgtctacaactcttatgtaggacgtaatcggaacaattatcgggctaggggctacttagctgttagacaatatcaagtaatgccttttgccttttctgttcttaataattatgagactaagctagcagaagaagtagtcgccaataatgagttattggctaagccaaagaacattcgcgatgcttatagctttttacgcgttaaagaaattgacagtctcgctattgccaatgcgattcaaaactatcagaaagcttggaacaactatcgcaagcttggtcatggaattccgacttttcacaaaaaacgtagtgattggtcatatcagaccaactgtcagtatcctaaacagacagaagcctttcttgataacgggacagctagatttattgatgctaagcacgttaaattgcctaagctaggcgttgtccgcattgccagctttagaaaattgattaaagagcgttttcttaatcatattccaactagaattggcacagtcacgattaaaaagaccgctgacgaccagttttacttatcaatgcaactaggcagcgatattgcctttgttaaggaattgcctaagacgcagagtcaaattggcatcgacctcaatcttgataatttcttaacagcatctaacggagcaatggtggctaatccacgattttatcgcaaaaccaaaaagaagcttgcccatgcccaacgtgtcttgtctcgcaggcagcgcagagctaagaaagagggacgcaatttgcgcttagccaagaattatcaaaaacagcgtctagcagtcgctaaactgcacgataagattcgcagacagcgaaatgacttcttgcaagtactctcaactgcactaatcaaaaaccacgatttagtagttgccgaggaattaagaagcaagaacctgttaaagaatcatgccttgtctcaagctatttctgatgttggctggcgcagttttctgaacatgctggcatataaggcagacctatatggcaaagagttcaaaacaattgatcctaagtacaccacccaaagatgccatcaatgtggcagtattatgggacaaaacggttataagaaattaacgcttaaggatcgagaatggacttgtccaatctgtcaaacgcatcatatccgtgattggaatgcggcagtaaacatcttagaaaaaggcttaggcaactggcagaattctaaaataaaaaaagcagcctaa

SEQ ID NO: 267 - RNA, 792 nt, synthetic construct
  source 1..792: mol_type = other RNA
atgaaatcttccatggctatgataaaacataaaaaggagctggttgaatatgcacggcacctgaaactaccgaacttggcagaacatgtgggtgtgatacttcatgaagcacaggagaagcaactgacctattcggagttcctggcgaattgtcttgccagggaaatccagggcagagaaaggaaaagctacctgacacggttgaaactctccggactccccgccaaatacgatctggacctgtacgactatgacagggcggaaggaatggataacaggaggctgcgcgaactgcgtgagctggtatgggtgaaccaggcatacaatcttctcctggtaggcccttccggaaccggcaagacctttatagccgccggactcgtgtatgaagccgtaaaagcgggatacgaagcatatctgatgacactggaggagctgctcacatgcctgaaaacgaaagaggtctccgcacatgcgatgaaaacatacaagcggatcatgaaggcccggctgctggccatagacgatgccacactgttccctttgaaaagagaagaagccgtgttgctgttcaaactggtcaatgacttccaggaaaggacatcactcatcatcacggcaaacaaggcactcacccgttggctggaaacattggaggatgaagcggtcacagccgccttgcttgacaggctgctctactgctgcgagattatcaggctcggaggaacaagctatcgcatgcaaaacaggaaaacaatttttagcaaccaaaacacggatataggcacgtaa

SEQ ID NO: 268 - RNA, 1893 nt, synthetic construct
  source 1..1893: mol_type = other RNA
atgaatgcttctagtaaaaggaaaattattagtcagagtgagattagcaaaaaaatagctgtaatgaatgaagaaatgcaggggttttgggctaataatagttgggatataagaaaatgtccacatccttctgccatagaattaagtaagaatcctgctttaaggaatcgttgggttcgttttgaacgtgttaaaaatctgtggttaagaacagaattgaaatatttttatttttaccatttaaacaatggaatatggaatgcaaaaactgtctggattagaaaaggaacagtaattaataaaatgttagatttcttagatttaaagtatcctagcattacttcaattactgaagttcctattgaaaaagcaatgacggagtatagaacttatttgacaaaacggggtgttagaattaccactactaattataagattactgctaatcaagaaaaaacacctgtaaaagctaattcctactatgttactaatctaaaacaatttatggagttttatgagaacttttattttgatggagaggagtgggataaagacgtttgggatagacgtaacttacctttgccagatgataaggttaacccaacacaatatgaatatacaattaactttaaagggtttcggaatacatattttaaacaacttgtaaaaagatattgtaagttgagattgaacgtggatagcttttcctatgtaagtgatattgcccaaagacttaaagagttctttaattttctggacatgaaatttaaacaagttcagagagtacaccaattaacgagagtggaaattgaagcatatttaagtgaactaaacatgatgggaataaaacctagtacaataactgggaggatctctatattggaaggactatttagtacccttcttaggctagaatgggatgatgttccttccaaaatattaatttattctgaggactatccgaaaataccaagagcaaaaccacgctttatagatgaattcgtcctagagcaattgaacagtcatcttgataaattacccgaatatatagctacgatgactatgattgttcaagaatgtggaatgaggataagtgaattgtgcaccttgaaaaaaggctgtctattagaggacaaagatggagatttctttttaaagtattatcaatggaaaatgaaaaaggagcatatagttccaatatctaaagaggtagctttacttattaaagttcgggaagataaagtttcagaggaatttccagatagtgaatacctctttccaagaaaagatggatcgccattaaaacaagaaacatttagaggtgagttaaataaattagcttatgagcaaaatatagtggataaatcaggtgagatttatagattccatgcccatgcctttcgccatacagtaggaacaagaatgattaacaacgggatgccccagcatattgtgcagaaatttttggggcatgaaagcccagaaatgacaagcagatacgctcatatctttgatgaaactctaaaaaatgaatttactaaatttcaggaaaaactggttaccaataatggagatgtgcttgatctagatgaagataatgaagtcgatgatgtagagcttcaatggttcaagaaaaatataaatgcacaagtgcttccaaatggttattgtagattgccagtagtagcaggtggttgtccacatgcgaatgcatgcttagattgcactcacttctgtaccagtaagcaattcttaccacagcacgaagaacagttagagcgtacagaagagttattagccatagctaaggataaacaatggcaaagacaagtagagactaatagccgtgttaaagagcgtttagaacaaatcattggaagtttgacggggtaa

SEQ ID NO: 269 - RNA, 1329 nt, synthetic construct
  source 1..1329: mol_type = other RNA
atgaaaaggatgagtagtttagcctaccactttggcattaagctgcgcttttatcctagctctaagcaaaagaagctaatcaagctgaactacgatgctcagcgttttgtttataattcttatgtaggacgcaatcggagcagctatcatgccaaacgctattttctcgtaaaacaaagcaagacaatgccgtttgctttttctattctcaatcgctatgaaacaaagctggcagaaacagtagtcgctaataatgagctattggctaagcctaagaatattcgagatgcttatagtttcttacgcatcaaagaaattgacagtctggcgctggccaatgccattcagaactatcataaggcttggaataattatcgcaaaatcgggcatggaatcccgactttccacaagaaacgcagcgattggacttatcaaaccaactgccaatacattaagcaggaagaagcttatttggacaacgggacagctaaatttattgacgctaagcacgttaaattgcctaagctgggtgttgtccgcattgctggcttgagaaagctaattaaagagcgtttgtttaagcacattccaactagaatcggaacagtgacgattaaaaagaccgctgaagatcaattctatttgtcaatgcagtcaggcagcgatttagcttttgttaaagagctgcccaaaacacagaatcaaatcggcattgatcttaatttagacaacttcttgaccaattctaatggtgccatggtcgctaatccgcacttctaccgccaagctgaaaagaagctcgcccacgcccaacgagtactctctcgcagacaaagacgtgctaagaaagcaaggcgcaatttgcggacggcaaagaactatcaaaagcagcgcctaatagtcgctaaactgcacgacaggattcgcagacagcgtgaagacttcttgcaggtcctctcaactgcattaatcaaaaaccacgatttagtagttgccgaggagctaagaagcaagaacctgttaaagaatcatgccttgtcccaagcgatctctgatgttggctggcgcaagtttcttaacatgcttgcctataaaggcaggctatacggtaaagagttcattactattgaccctaagtacacgactcaacgctgtcatgcttgtggcacaatcatgggacataatggctataagaaattaaaactaaaagaccgtgaatggacatgtccaatttgtcatacttaccatattcgtgattggaatgcggcggtaaacatccttcaaaaaggattaggcagttggcagaatcctaaaaaacaagccacttaa

SEQ ID NO: 270 - RNA, 1266 nt, synthetic construct
  source 1..1266: mol_type = other RNA
ttgcttaaagcaataaagtttagaatttatcccaccatagagcaaaaaaccttgatacacaagcactttggctgtgctagggtggtctataactactttttagcataccgccaaaagcaatacgcacaaggcattagagaaaattactttagcatgcaaaaggcgctcactactctcaaaaaacaagaggcttacgcttacctaagtgaatgcaactctcaaagcttgcaaatggcactaagacagctgacaacagcctttgataggtttttctctaagctggcagattatcctagattcaaatctaaaaagcattctaagcagtctttctgtgtcccgcaacacttagagatggatttaggcaacaaccaagtcaagctacccaaattcaaagaagctattaaagccaagtttcataggcatttgcctacaaactctatcgtcaaacaggggtttatctcttgtgtagcagataaatactatctctctataagctatgaggataatgagcctgaacctaaacccacaactatcagaaaagctgtgggtttagatatgggtttagagtctttagtcatcgccagcaatggcgtgttctacccatataaaaagtttttccagaatctacaaaccaaactcactaaagcgcaaaggagattgtctaagaaactaaaaggttctaataatagaaaaaaacaagctaagaaagtggcacaaatccacgcttctatcaggaatagcagagaggactacctacataaaatcagtaatgagataaccaatcaatacgatttgatagcagtagaaaccttgaaagttaggaatttggtcaaaaaccacaaactcgctaagagcattgccaatgccagctggtctaggctcattagtctattagaatataaggctggttggaagggtaaaacccttatcaaaattgaccaatacttccctagctctcaaatctgctcgacctgtgggagcaacacaggtaaaaagccactgcctattagaaattttatctacccttgttgtcaaacataccaccacagagacctaaacgctagcatcaatatcagaaactatgctttgggaatgctagatgagcgacacgctatcaaggtagataaaactagggtagggattacccgaagttacgcttgtggagattccgctaacggggctgtaaccaagtatggctacatactggatactgctagttatggatcgttgaagcaagaagcccacccgtctttagcgggtgggtga

SEQ ID NO: 271 - RNA, 500 nt, synthetic construct
  source 1..500: mol_type = other RNA
atcttgccgtccgtacatggacttgcccgaactgtggggaaacccatgaccgagacgagaacgctgcgctgaacattcggcgtgaagcgttggtggctgcgggaatctcagacaccttaaacgctcatggaggctatgtcagacctgcttcggcgggcaatggtctgcgaagtgagaatcacgcgactttagtcgtgtgaggttcaagccggcaatcacgcccgagttgctgcccgagccgtcgcacgccagcaccttgacggcgatcagcgacacgcccttggcgacgccgtaggtgctgctgcccaccgtgccggcgacgtgggtgccgtggccgttgcagtcgctatcgttgccgtcgcccgactggttggtgccccacacggcgcggccaccgaacgccgtatgactagtgttgatgccggtgtcgatgatgtacgccttcacgccgctgccggtggtgccataggtgtagctgctgtcgagcggcaggctgcgct

SEQ ID NO: 272 - RNA, 500 nt, synthetic construct
  source 1..500: mol_type = other RNA
acaaagcttctcactttgaaaataccacagtaatcgaagtggatgaggaatttacgacaaagacgtgctccttgtgtggtgtccgcagtaatatcggtgcgtccaaagtgtttaagtgcgataattgtgacgcgactttcgatagagaccacaatgcggccaaaaatatccttttgaagtaccttggcgagatagattagtatgctatatgtagtgcttcctgtgtatgctgcaatgaatcgatgtttcatgagttaattttgtttttaccttttcgggaaatgctggcaatctaattaatcactgatctggcatactaagttatgcagccatctcttctgagaacggttcgtccgtcaaatgtataaatacagacgccatcgctctgttgtttgcactttcatatcttcctttagacctcgagcctggcttcttttcgaagttggtcctcgggtccccgccagctgcttagcagaggtcgtgaacatcatgaggcgatg

SEQ ID NO: 273 - RNA, 499 nt, synthetic construct
  source 1..499: mol_type = other RNA
atgatctaggatctaataaaatctacagctgttcaagttgctcctcggttttcgatagagacttcaacgcagcaaagaacatcatgctcaagtatatctgcgagcatttgatggcaagtggtggatcttccattacctctgcttcaagtggaggaaatggtggattcgccatgatgggtcctggccccttcgtacgttagtacgttgtaagtcccttgtggattctcccaggcgctacccgcggaacgtattactaagggtatatatataataaggataatatataaagtgctgcaaaagatgaggctcgaatttttcacaagttatcggtcccaacgacctaccagttaagaaagaccaattaaggaatatggttacttccagagctgtcacttttatcaataattctacacctgctactttggtgaaaaaagagattgaccttgataaatgtcatgatgaaaatcaaattgttatacaggtttatgctgccagtttg

SEQ ID NO: 274 - RNA, 500 nt, synthetic construct
  source 1..500: mol_type = other RNA
tggcccatgaaacccagaaccatctccagctcaacttttaccggcgcttcaaacaatacattacgctccgctacgggctggctggccgcgatcgctaccttgtgctcaagggtatcctcgaccccgagtacgacggagacaacgagatcgtgctggagtacagagaattgatcccccgcaatgatgaaggagcgagctagcgagctactcccgcttgtgcacatgttcctcactttcgtgaaggaccacaaccgcgagcacgaaggagaaccgggcttcgagatggcaaggacgtactcgctactaccaacgaaacggggcttcgtctgctctcacttcaccatgtcctctcgcggcttgcgggcgatgctggttcgaagtggatatgatgttccggaagagggtagcgactggaacgctgtcgcggacagcatgtggcggcggctcttcaacacgaaccttgtcgagacgtccaagccggacgggtctccaagtcgctt

SEQ ID NO: 275 - RNA, 499 nt, synthetic construct
  source 1..499: mol_type = other RNA
tgactgatgttcagggtggtcagcgtgtgcgacagtgtacaactgtttccgtgggcgttgaaaatccctgtcctcttttcgaaaacggggtggcatttaggtgtgatagggatgcgagtgccaccctcaacttttgtctcgcgggatactgtgggttggttcgcaagtcgtggccaactcatctcctgagacgtcagtaagaggacagaaagtagtccctctgggacatggccggtagattgacgcagatgtaaggccaggcctctcattcacacgagaggtctattgttggacgagtacgttggttggatcattttggtcttgttaactgtattcctccgtaatagggcggatgtagccacctgtacatattttcttgtgcgctcctctacaggagacacgtgcttgtcttgattgaagtctgttacaaggtattctgcgccgaggtaataggttattagaccagagatgacaaaggcggtggcggcgtaagcgtagc

SEQ ID NO: 276 - RNA, 499 nt, synthetic construct
  source 1..499: mol_type = other RNA
gcccggggtgtggagaggacatggtcgaggacaaagaacggagaatccggagttgcaaaaactttaagatcggatcaccggaagagtcctgtagacttcaccgcctgacaccagagtttgaaatggatcgcgacgacgtagggtcgactaatatcggtatgcgtggtttcggcaaattgctcggtcaggtgtggttttagggattttttccggattttcacttgcggatgatactttatagttggttttcctaaagttgacaatggtgagcgcctccacgacatccttttgctatgcgccttccgtaacgcgctgacctccgtggccaagatgcttgctgtggaagcatcttctgtggcaggaccgttgtacgtgactcgctctttggacttgacctcgagaactgaaagcagtgcctgccagaagcaagggtcaaactgctcgattgctgggagacctcccacatctccgtccacaacaactcggtcgaacagctttt

SEQ ID NO: 277 - RNA, 500 nt, synthetic construct
  source 1..500: mol_type = other RNA
aaacgccagtaccttcttccacttcctcttccttttgtactcctacttccattacatgtgtcttgggaggaaaattcgtcgacagggacttcaatgcaagcaccaatattgttcataaatttctagggttttgggataaaaagttaatggaaaagaaagacaagatgccgttgaagtatcactttattcgagttgcctgagaataggatgtaaaaaactcgtgtcactagtgaaatagtggcgtagccctgaaggatgagaagtatgctattttcgaccttacggtcattgtggtatatcaactcttgcccgataaagggttattcaacaacataaaaatggtacaatattctaggtgtgttttactattctttgcttgaaatcccaaaaaggtgacccttcaagttcacttgatacactaacttcaacaaatggggatatttctgccttcaccttttgcacaacagactctatgaatttattacacatcaaattcaatt

SEQ ID NO: 278 - RNA, 499 nt, synthetic construct
  source 1..499: mol_type = other RNA
ctcaagcctgttccacgtgtaaccagcagactggagagtttagtaaaattgtaatggatcacaagaagcgagtgggagtttgcccaaactgcgccagccgcttcgatcgtgaccagaacgctgccaaaaacattttaattaactttatgaggtctaccgtctgtaggccaagatgctcttacactaaaagggtatcgtagcttcctgaaacgtaaatgttgtaaaatcaaacgcaataaggttcaatttcaaattggcatttatttgcgtttggttctcaggaggttatctacacgtcgacaattttgccatgacatacttggtttcatgtcggaactctcgctcacacccaaacaatgcacagcacaacagattgttacgctgctggtgtgtacgagcgccctcaaaatcataatctttgtcgtcaatcaatttacacacaaagcgcagatactgcgactcgtaaggctcgggtagattgcgagatttcataaagtcc

SEQ ID NO: 279 - RNA, 500 nt, synthetic construct
  source 1..500: mol_type = other RNA
cttcacaagcctgctcagcgtgcaaccataaaagtggagtgtttagcaaaataagcatggatcacaagaggagaaggggagtttgcccaaactgctccagctcgttcgatcgtgaccagaatgctgccaagaatattttaatcaactatttaaggtctgccgtctgcaggccaagatgctctataagaagggtatcgtagcttcctaaaacaaaattgttgtgaaatcaaacgcaataaggttcaaattcaatttggcattatttgtgtttggttcttaggaggttatatttaccacataaaatatacaacttaaccaattggttttgattaaaatctgcactcgactcgctcatttttgcaccataacggtacaatagaaataaaattttattgacaaataagtttgctgtaaatttgttcatgtacttaaaggtggtctctatataacatggtaaataagctgaactgtatttttttaagagatatctttcaattgaa

SEQ ID NO: 280 - RNA, 500 nt, synthetic construct
  source 1..500: mol_type = other RNA
gacttcgtcgcggcgtctgtccagactgcaacgccgacgtcgagagggactacaacgccgccaaaaatatactagttaattatcaaaggtccgccgtctgcaggccgagcccgagctgtgactcgggcgtagccaccgacacggacccgcactccatacggtccattttccaatgcgccgtatcggagcccgggtcttaggaggttaccgtgcacccttctcactcggacgtggggtgattgcgagacgtgcggccaacgcacgaatcgtgctacgacgtgtcgacgacaagtgtgccaccgttgccatcgttatcacgaacattgtcagtactgtgaacgcagtagtgagacgtcagtcgactcgtaaacatgatactgttcgttacgtgtttgttcgtcaccaccgtttacggcgattgctcgactacgacgctgtcgtttgtccgaaaaaccggaaactgcttcgattacgaacagggatcggtgagccatcccacg

SEQ ID NO: 281 - RNA, 499 nt, synthetic construct
  source 1..499: mol_type = other RNA
gtcgcggcgtctgtccagactgcaacgccgacgtcgagcgggactacaacgccgcgaagaatatattagttaattttcaaaggtccgccgtctgcaggccgagcccgagctgcgactcgggcgtaaccaccgaacgtgactcgcactcgatgacgatacggatcatttttcaatgcgccgtatccgagctcgggtcttaggaggttatgtatgtgtcattaggttacaagtcattaagacaaaatgagtaacggggaaaacaaaatcgaatgggatgtaaggcgtggcgtggccctcagtcaccttcgaaagcgtaatgaaaattcgatacgtcaaccagcgttacaaaaccctaaaactcttgatatgattagtacgcttgcgttgaataacgcaagcgtactacatggtgaaatgcttcgaacacttcgacgttcaccaccattgttgcgagaaaaacttttgtcttctatatcgacagtgccgttacgagtaacgc

SEQ ID NO: 282 - RNA, 499 nt, synthetic construct
  source 1..499: mol_type = other RNA
tagtgtgttctaactgcgtgagttgcgaaaacaaaaaaatcgtatttagaaccagagatgctaattcctcaataaatataatgaaattaacaagttgttggatagatagccaaactcgtccaactgcatttcattctcatatttcgtctttcacctgttccactatcaaagaagaacaggaaaaagtaagaccatcgtaggtgaaattcctactattgattttacattttttcttatttttttgccctgtaaaatgggcgtttgaaatgagaaaaggtgtaaagcctctaattcaaactgtggttgagtctggaaccgactttttagacaaaaacgttcattattttgtatataacaagcagcgtgatacacagaaaattttcaatatgctgtctgcgtgccagtgttgcgagcgtcatcaagtggataaaccaagagcaatggagccgtgggttgaaaccaagtttaatatgacgcaaacaactccttgtgaatgtat

SEQ ID NO: 283 - RNA, 499 nt, synthetic construct
  source 1..499: mol_type = other RNA
gttataagtatggcactgaagtggttaatgtcaatgaatatttgaccacaaaaacctgctcaaactgcggcaagattaaagatcttggagctagcaagatttacgaatgtgagtcttgtggaatgtacgcagatcgtgatgagaatgctgccaagaatatcctaaaagtaggattaaagccttggtacaaacaaaaatagtctaataaaatcaggggtacattccgctagtactccaccctacgggttaagcaaatgagaatatcgaaacggtatgcacaggattcttcgagtgataatcttaggatgactcactaaggagatgactaaagtgtatcattcaatattgtattgaacggtattcttccatagagagttgatttttggagtatccagaaatatcaactttttatgagcggcgtttgtgttatcggtagtaaatatacctatgaataaatatttggttgaacatctttccattaactataaacataaaagag

SEQ ID NO: 284 - RNA, 500 nt, synthetic construct
  source 1..500: mol_type = other RNA
caaaatgtggtcgtattaacgaaataggttcaagtaaagtccataaatgtgcgtgtggaatgtatgcaggtcgagatgaaaatgcagctaagaatattctgaaaaaaggaattaaagaacattatttatcagctcaaagtgataaaaatgttacagaatataacaacaaaaagtctaacaatacaaacaaagaaacttagtagccttcatctcatcgtattaggattacgctctcatcctaacagtatttcggattaaccccccgatttgatagagcgaacacataattaataaacttattggtttatcaaagtcaactttaataaaccaatagcaattgatttgattgactattacgatacaaaaaaaattgaaaaaaaaacattctgatataaaaaataaatagtagtaatagattacggggggttaatccgaaatactgtacagataagactaattacaagagagattaatgagacttttttaaacaacaatcacaa

SEQ ID NO: 285 - RNA, 500 nt, synthetic construct
  source 1..500: mol_type = other RNA
aatgtgaggagtatggtagtcaatactatgaggtagatgaatcgtttacgagtaaaacatgctgtaaatgtggaaatatacacaaaacattaggatcatcaaaaatatatgattgcataaaatgtaaaaacaaaatagacagagatgtgaatgcatcaatatgtattctgataaaaaataaagatatactattaaaataaaaaaaacaaggagagcggtctgctccttaatgggaaatgtgacaaatgtataacaaatgtctataattaacatagattcatacatttacatattttttaagtgatcggcaatatctaatgtatcagttaataatatatccgttgatgtatttaaaataatattttgctgatcacaataattttgtatctttttatctctttttattgaataaggtgtataatcatcattaatgtatattgcagatatattaatgtttttatgtatttcatcgattacatctatttcatcaccatatgttg

SEQ ID NO: 286 - RNA, 500 nt, synthetic construct
  source 1..500: mol_type = other RNA
tggaagccaaaaaagttcataaatgcaaatgtggaatgaaaacagatcgagatgttaatgcagcaaaaaatattctcaaagtaggatatcagtataacagtactctgcaccaagttaacgatatagatattattgaaacagacaatcatatagttacttttaataaagtgattaaagaatatgaagttttcgaagtataaaaaatatcataggcaaattccgctagagccttaaatgaaattattaggatattgaattaaattctatgggattcaatttaaatattcttgaccaataattctattggaataattttaatggatcactttaaaaaaaattgatattttcttttaaatatcaataaatataaagttttctaaagcggatcatgtgaatttatactcattataaaataaaatgataatatattagatttttatcaatctaatatatcatacaaaaattatggattgattcgggatttaatatatcaatttttt

SEQ ID NO: 287 - RNA, 499 nt, synthetic construct
  source 1..499: mol_type = other RNA
gtgatacgattctcgaacatcccggagagcaatggacttcgaaaacatgtggaagatgtggaaagtcgaatcttccagggagagaacgtcgaggaactttcagagagtataaatgtctttattgcggattgcatgcgcatcgagatgtccatgcggctcgcaatattttcatgaaatggtatatcgaactgaaaaaataatataaatcatcgggttagtcaagtgctccgagaggagacaatcccttacattgtggaaaatgaaagtatttcgatacttttaattaccatttgatgtataaaatgaacggctgcagcacgatttttgaaagtaaaaaatggttcacatatttcaaaatgtataaataatcccacaaaatataaatcggcatataaatacttctggcgccggggatccagctgatattctgacagctgtgttaccgagtaaaacagggccaaagctcgttgttgaaaaaggtgattatcgcaggttcaac

SEQ ID NO: 288 - RNA, 500 nt, synthetic construct
  source 1..500: mol_type = other RNA
cagaggaatacacttcgaagacatgtggtgtttgcggcctgattaattatctgttgggcagcaagaagacattcgagtgtttctgcggcaatgtatgcgatcgcgatgttcacgcagcccgcaacatcctcctgaaatggctgagctccgaggcaggtgcccgggtcgcagagaccttccttgcctctcggtcgagttagacctcttggtgacgatacttatatcgacactgagtgcttatattattcggcgtatacggatgatatccatagatattatctgctagaataatatttgattaacttatatgttagtctttagtaaatttcttatgaaaacagaaccattcatgattcatctcgtggttctcgcgatgttcgtcacgatctatcgtttcttccctggcggattcgaaaataacttttctgttgcaaatccggacaaaaaggcatcatggatagattgtttatatttcggagtaacgacacactcgaccgtcg

SEQ ID NO: 289 - RNA, 499 nt, synthetic construct
  source 1..499: mol_type = other RNA
aatttagagaaatattagaatatcaatgtaataaatataatactcaattaaatatagtagatgaatcatatacaacaaaaacttgtagcaattgtaaggagttaaataatataggttcatcaaaaaaatatcaatgtaaaagttgtaattttaaatgtgatagagatataaatgcatcaataaatatttatttaaaataactaataattataggcatgctacttgtcttgaataaaagttaggtaattttctacgaattattactgtttttataaataaatataaacttttaagtagcgggaatttgatagaaacatgtattaatacaaataatagggacctagaaaataatattgatattatagattataataaagattttattgtgtctaattatgcaacagaattatttttagaaaaacttgataatttaaaagaatttagtttaacactagattcttttaagttattggaaagtgatcttattcatattaaaaat

SEQ ID NO: 290 - RNA, 499 nt, synthetic construct
  source 1..499: mol_type = other RNA
cccagaaattcacggatgtcaaggttcgcctatgtaacgaggcatatacgacgaagcaatgtgggtcatgcggggttatcaatcgctccatgaccttgaacgaccgttcattcaactgtgtgtcgtgtggtctctcgtcttcaagggatggtcatgccgcgaggaacgtcggtcttcgctcgttacgattccttgtctaaatgatctaggcaaatgtagatacgggggctcctattgctcccttgacggatacctggaaagtcataaagaaatacattggttagtctacccggagtgaatgtagagggttttctatgggtttccaccctttaccgtattgaccataggagctcgctagcttcgtccaggacgaattgagtcgttcccgggttaaactccggaaaagagagagagagagagagagaaatggaaggggggaggacgagaaagaaggaataataataaacatacgtggagcttacggtgtgataaaacatac

SEQ ID NO: 291 - RNA, 499 nt, synthetic construct
  source 1..499: mol_type = other RNA
ttgttaatagtgtaaaaggattgacaatattaacaccaaatataaataatttaccatttcaagtttgctgggataaaaatagaaaatttaaatatgaaaacataaacaaaaagataaaactatttattgattgcttacaaaaaccaaaaccaagttggaaagaaacatttatgtcaaatatacgaacattagatttatagatattcacttggaatataagaattacatatttgtctatgtcctattttaaaccttccagaaaacataaaattatctttgaatgtattgctatttatgtatgatactatattatttaaattgcactttttttttggtttaagcattattaatccaccaccaaaataattaactttacctaaaaacgatacatttggtttccgtgttaaattataaatgtaaatacaatctttaccgagattagtgtttatggtagtaatatttcttggtgctccccattcaaaccaattattttcattaa

SEQ ID NO: 292 - RNA, 500 nt, synthetic construct
  source 1..500: mol_type = other RNA
caaatgtatcgtttttaggtaaagttaattattttggtggtggattaataatgcttaaaccaaaaaaaaagtgcaatttaaataatatagtatcatacataaatagcaatacattcaaagataattttatgttttctggaaggtttaaaataggacatagacaaatatgtaattcttatattccaagtgaatatctataaatctaatgttcgtatatttgacataaatgtttctttccaacttggttttggtttttgtaagcaatcaataaatagttttatctttttgtttatgttttcatatttaaattttctatttttatcccagcaaacttgaaatggtaaattatttatatttggtgttaatattgtcaatccttttacactattaacaattatatcactcgcatcggttttatttaacactatgaaatagtaatcttttttgttattagtgttatattttttattttttaatttattgaaaagtatatcactcat

SEQ ID NO: 293 - RNA, 500 nt, synthetic construct
  source 1..500: mol_type = other RNA
atacagcgaaagagaaccgcctgtcacaaagtaaattcagatgccaggtatgtggatatacagcgaacgccgatgtaaatggcgctcgcaacattttagcggcggggcacgccgttcttgcctgtggagagatggtgcagtcaggccgcccgttgaagcaggaacccaccgaaatgattcaggcgacagccagaacgtagcagggatccacgtccttcagggcgtggaggatgtcaatatcggtttctgtcagcgattaaattcaacaccacgtaacaaacgcctccggcaattaatccccaaaacgcagaaccaatcccgaccagcgtcaatccacttgccgttaccagaaatgccaccaccgccgcgtctcgctcacgctcattatgcagcgcctgatacaaactgccgccgatggtacttaacagcgccagacctgccagcatctggatccaacttacgggcagggcagccatcatcccggtaatggcactaccaaa

SEQ ID NO: 294 - RNA, 499 nt, synthetic construct
  source 1..499: mol_type = other RNA
gttataagtatggcactgaagtggttaatgtcaatgaatatttgaccacaaaaacctgctcaaactgcggcaagattaaagatcttggagctagcaagatttacgaatgtgagtcttgtggaatgtacgcagatcgtgatgagaatgctgccaagaatatcctaaaagtaggattaaagccttggtacaaacaaaaatagtctaataaaatcaggggtacattccgctagtactccaccctacgggttaagcaaatgagaatatcgaaacggtatgcacaggattcttcgagtgataatcttaggatgactcactaaggagatgactaaagtgtatcattcaatattgtattgaacggtattcttccatagagagttgatttttggagtatccaaaaatatcaactttttatgagcggatacattagaaacagaagagaaatactgaaatatttgcgccagaaaggatacgttatctagattttcaactaaaaggattt

SEQ ID NO: 295 - RNA, 500 nt, synthetic construct
  source 1..500: mol_type = other RNA
taaatgcggacgtgaatgcggtattcaatcaacactttctttacctgtcccatctcctaaatggaggtgggaaagcccgccctgtggtgcgggttgggacttcgctgaaaagtccctcacgtgagggacacaatctaagcggagtccctaaagcaacaactacctttttctatatttcttcacttttgtctacattttaaggtagctgttgcaaccactcatcaataactcttctagttttctaagcattcctttacaaccgagcggtttatccaaaataaggcaaacacccttctatcaagtttccctttataggttattcccttttcagagaaatagctttagagggaagttttcttccctaaacatacgcttccatcttcagttgtccattatcatggacaacgaaatagataaatagcttagaagataaatatttgacaaagtaagcagaaaagtagacatttagaattatgacggttaaacaagaagacaagctt

SEQ ID NO: 296 - RNA, 500 nt, synthetic construct
  source 1..500: mol_type = other RNA
gttataagtatggcactgaagtggttaatgtcaatgaatatttgaccacaaaaacctgctcaaactgcggcaagattaaagatcttggagctagcaagatttacgaatgtgagtcttgtggaatgtacgcagatcgtgatgagaatgctgccaagaatatcctaaaagtaggattaaagccttggtacaaacaaaaatagtctaataaaatcaggggtacattccgctagtactccaccctacgggttaagcaaatgagaatatcgaaacggtatgtataggattcttcaagtagtaatcccaggatgactcactaaggagatgactaaagtgtatcattcaatacttcattgaacggtattctttcatagagaattgatttctggagtatccagaaatatcaactttttatgagcggtatatacatcacattattagactaatgattatttgtaatttcatgaatatttgtatgatagatccgaatgatgtttataaat

SEQ ID NO: 297 - RNA, 500 nt, synthetic construct
  source 1..500: mol_type = other RNA
ctaagtacaccacccaaagatgccatcaatgtggcagtattatgggacaaaacggttataagaaattaacgcttaaggatcgagaatggacttgtccaatctgtcaaacgcatcatatccgtgattggaatgcggcagtaaacatcttagaaaaaggcttaggcaactggcagaattctaaaataaaaaaagcagcctaagctaggctgcagtccgctagcaactcgcggactcaaaaggctttggtaattagacgacctctatcggcaatagtcaaaagattattgttggtaagtcaagtccgtatctaagcaagttggtgttctggttagcgcaagctaacgagccagccaccaagccccagaatttattctggggtagttgacttatcttgagtctatttacgggaagctattcaattcggcagattaatccgcttgaaaaagtgaagttcaagtatgtgcaatggctatctttggctattgggctggtctgtttaa

SEQ ID NO: 298 - RNA, 500 nt, synthetic construct
  source 1..500: mol_type = other RNA
aggaaaggacatcactcatcatcacggcaaacaaggcactcacccgttggctggaaacattggaggatgaagcggtcacagccgccttgcttgacaggctgctctactgctgcgagattatcaggctcggaggaacaagctatcgcatgcaaaacaggaaaacaatttttagcaaccaaaacacggatataggcacgtaaaaagagttaaggaaagtgaagcatcttcgatgctggagtgggatatactaaattacctaaaaaagtggcgcacaaatttgcgcgccacaataatggcaatttattaatcaatagatggactgaatcacctatgacagtatctcattttaatgatctactaataacttgtaaaggtactattggtgaaatggcttttaatagaatcggtagtattcacatagctcgccaaataatggcattaactggcattgttggtgacttggattatattagatggttcctttatgccaatgtatcatc

SEQ ID NO: 299 - RNA, 499 nt, synthetic construct
  source 1..499: mol_type = other RNA
gttgtccacatgcgaatgcatgcttagattgcactcacttctgtaccagtaagcaattcttaccacagcacgaagaacagttagagcgtacagaagagttattagccatagctaaggataaacaatggcaaagacaagtagagactaatagccgtgttaaagagcgtttagaacaaatcattggaagtttgacggggtaattatcaatggataaacaagttagaaatacaacagaaattgtacgtttggcgaagcagaaatcaaaaaagacaagggaaaaagtagacaaagcgatttctaaattttcgattgaaggtaaagttattaattttaattcaatagcaaaggaagctaatgtttctaaatcatggctttataaggaacacgatattaggcaaagaatcgaatcccttcgtgagcgtcaaataacagcaaatgtagtctcaaaacccaagaaaagttctcgttcggaggaaatccttattaaaaccttaaaaag

SEQ ID NO: 300 - RNA, 500 nt, synthetic construct
  source 1..500: mol_type = other RNA
accctaagtacacgactcaacgctgtcatgcttgtggcacaatcatgggacataatggctataagaaattaaaactaaaagaccgtgaatggacatgtccaatttgtcatacttaccatattcgtgattggaatgcggcggtaaacatccttcaaaaaggattaggcagttggcagaatcctaaaaaacaagccacttaaaaaggttggaagtccgttagcaactcgtggactaaaaaggctttggtaattaggcgacctctatcactaatagtcataagaatattagtgataagcccagcccgtatctaagcaagttggtgttctggttagcgcaagctaacgagccaccaagccccagaatttattctggggtagttgacttgaacaatttggtacaggaattcaaagaattcgtgcagcgtatgctgaaagtaaaattcagccccagtttttaatttttgaaaattcaatcaagattgttttaccggtaattaaaaa

SEQ ID NO: 301 - RNA, 500 nt, synthetic construct
  source 1..500: mol_type = other RNA
tcaatatcagaaactatgctttgggaatgctagatgagcgacacgctatcaaggtagataaaactagggtagggattacccgaagttacgcttgtggagattccgctaacggggctgtaaccaagtatggctacatactggatactgctagttatggatcgttgaagcaagaagcccacccgtctttagcgggtgggtgattcactaccctagttaatgatgtaagaaatatttggcaagattatgccaatagcattggcaaagatataaataccgccattcaagaggcttttacaaatctaagcaaaaagaaacaagacttcactacatggctttttgacttcaagggagagagtgataagaagtttcaatacattatggatttggctaaccgcgagaaagaaagaatcttaagaaatctcaattatggggaggaggtgaacatggataattactctgtggtgttttctttggtgtgatctttgtcttactcctcttaa

SEQ ID NO: 302 - protein, 7 aa, unidentified
  source 1..7: note = Simian vacuolating virus 40
PKKKRKV

SEQ ID NO: 303 - protein, 16 aa, synthetic construct
KRPAATKKAGQAKKKK

SEQ ID NO: 304 - protein, 9 aa, synthetic construct
PAAKRVKLD

SEQ ID NO: 305 - protein, 11 aa, synthetic construct
RQRRNELKRSP

SEQ ID NO: 306 - protein, 38 aa, synthetic construct
NQSSNFGPMKGGNFGGRSSGPYGGGGQYFAKPRNQGGY

SEQ ID NO: 307 - protein, 42 aa, synthetic construct
RMRIZFKNKGKDTAELRRRRVEVSVELRKAKKDEQILKRRNV

SEQ ID NO: 308 - protein, 8 aa, synthetic construct
VSRKRPRP

SEQ ID NO: 309 - protein, 8 aa, synthetic construct
PPKKARED

SEQ ID NO: 310 - protein, 8 aa, Homo sapiens
PQPKKKPL

SEQ ID NO: 311 - protein, 12 aa, Mus musculus
SALIKKKKKMAP

SEQ ID NO: 312 - protein, 5 aa, unidentified
  source 1..5: note = Influenza virus NS 1
DRLRR

SEQ ID NO: 313 - protein, 7 aa, unidentified
  source 1..7: note = Influenza virus NS 1
PKQKKRK

SEQ ID NO: 314 - protein, 10 aa, unidentified
  source 1..10: note = Hepatitis virus
RKLKKKIKKL

SEQ ID NO: 315 - protein, 10 aa, Mus musculus
REKKKFLKRR

SEQ ID NO: 316 - protein, 20 aa, Homo sapiens
KRKGDEVDGVDEVAKKKSKK

SEQ ID NO: 317 - protein, 17 aa, Homo sapiens
RICLQAGMNLEARITII

SEQ ID NO: 318 - skipped

SEQ ID NO: 319 - protein, 6 aa, synthetic construct
GGSGGS

SEQ ID NO: 320 - protein, 9 aa, synthetic construct
GGSGGSGGS

SEQ ID NO: 321 - protein, 12 aa, synthetic construct
GGSGGSGGSGGS

SEQ ID NO: 322 - protein, 15 aa, synthetic construct
GGSGGSGGSGGSGGS

SEQ ID NO: 323 - protein, 18 aa, synthetic construct
GGSGGSGGSGGSGGSGGS

SEQ ID NO: 324 - protein, 21 aa, synthetic construct
GGSGGSGGSGGSGGSGGSGGS

SEQ ID NO: 325 - protein, 24 aa, synthetic construct
GGSGGSGGSGGSGGSGGSGGSGGS

SEQ ID NO: 326 - protein, 27 aa, synthetic construct
GGSGGSGGSGGSGGSGGSGGSGGSGGS

SEQ ID NO: 327 - protein, 30 aa, synthetic construct; identical to SEQ ID NO: 145

SEQ ID NO: 328 - protein, 33 aa, synthetic construct; identical to SEQ ID NO: 146

SEQ ID NO: 329 - protein, 36 aa, synthetic construct; identical to SEQ ID NO: 147

SEQ ID NO: 330 - protein, 39 aa, synthetic construct; identical to SEQ ID NO: 148

SEQ ID NO: 331 - protein, 42 aa, synthetic construct; identical to SEQ ID NO: 149

SEQ ID NO: 332 - protein, 45 aa, synthetic construct; identical to SEQ ID NO: 150

SEQ ID NO: 333 - skipped

SEQ ID NO: 334 - protein, 6 aa, synthetic construct
GSGGSG

SEQ ID NO: 335 - protein, 9 aa, synthetic construct
GSGGSGGSG

SEQ ID NO: 336 - protein, 12 aa, synthetic construct
GSGGSGGSGGSG

SEQ ID NO: 337 - protein, 15 aa, synthetic construct
GSGGSGGSGGSGGSG

SEQ ID NO: 338 - protein, 18 aa, synthetic construct
GSGGSGGSGGSGGSGGSG

SEQ ID NO: 339 - protein, 24 aa, synthetic construct
GSGGSGGSGGSGGSGGSGGSGGSG

SEQ ID NO: 340 - protein, 27 aa, synthetic construct
GSGGSGGSGGSGGSGGSGGSGGSGGSG

SEQ ID NO: 341 - protein, 30 aa, synthetic construct; identical to SEQ ID NO: 159

SEQ ID NO: 342 - protein, 33 aa, synthetic construct; identical to SEQ ID NO: 160

SEQ ID NO: 343 - protein, 36 aa, synthetic construct; identical to SEQ ID NO: 161

SEQ ID NO: 344 - protein, 39 aa, synthetic construct; identical to SEQ ID NO: 162

SEQ ID NO: 345 - protein, 42 aa, synthetic construct; identical to SEQ ID NO: 163

SEQ ID NO: 346 - protein, 45 aa, synthetic construct; identical to SEQ ID NO: 164

SEQ ID NO: 347 - protein, 4 aa, synthetic construct
GGGS

SEQ ID NO: 348 - protein, 8 aa, synthetic construct
GGGSGGGS

SEQ ID NO: 349 - protein, 12 aa, synthetic construct
GGGSGGGSGGGS

SEQ ID NO: 350 - protein, 16 aa, synthetic construct
GGGSGGGSGGGSGGGS

SEQ ID NO: 351 - protein, 20 aa, synthetic construct
GGGSGGGSGGGSGGGSGGGS

SEQ ID NO: 352 - protein, 24 aa, synthetic construct
GGGSGGGSGGGSGGGSGGGSGGGS

SEQ ID NO: 353 - protein, 28 aa, synthetic construct
GGGSGGGSGGGSGGGSGGGSGGGSGGGS

SEQ ID NO: 354 - protein, 32 aa, synthetic construct; identical to SEQ ID NO: 172

SEQ ID NO: 355 - protein, 36 aa, synthetic construct; identical to SEQ ID NO: 173

SEQ ID NO: 356 - protein, 40 aa, synthetic construct; identical to SEQ ID NO: 174

SEQ ID NO: 357 - protein, 44 aa, synthetic construct; identical to SEQ ID NO: 175

SEQ ID NO: 358 - protein, 48 aa, synthetic construct; identical to SEQ ID NO: 176

SEQ ID NO: 359 - protein, 52 aa, synthetic construct; identical to SEQ ID NO: 177

SEQ ID NO: 360 - protein, 56 aa, synthetic construct; identical to SEQ ID NO: 178

SEQ ID NO: 361 - protein, 60 aa, synthetic construct; identical to SEQ ID NO: 179

SEQ ID NO: 362 - protein, 5 aa, synthetic construct
GGGGS

SEQ ID NO: 363 - protein, 10 aa, synthetic construct
GGGGSGGGGS

SEQ ID NO: 364 - protein, 15 aa, synthetic construct
GGGGSGGGGSGGGGS

SEQ ID NO: 365 - protein, 20 aa, synthetic construct
GGGGSGGGGSGGGGSGGGGS

SEQ ID NO: 366 - protein, 25 aa, synthetic construct
GGGGSGGGGSGGGGSGGGGSGGGGS

SEQ ID NO: 367 - protein, 30 aa, synthetic construct; identical to SEQ ID NO: 185

SEQ ID NO: 368 - protein, 35 aa, synthetic construct; identical to SEQ ID NO: 186

SEQ ID NO: 369 - protein, 40 aa, synthetic construct; identical to SEQ ID NO: 187

SEQ ID NO: 370 - protein, 45 aa, synthetic construct; identical to SEQ ID NO: 188

SEQ ID NO: 371 - protein, 50 aa, synthetic construct; identical to SEQ ID NO: 189

SEQ ID NO: 372 - protein, 55 aa, synthetic construct; identical to SEQ ID NO: 190

SEQ ID NO: 373 - protein, 60 aa, synthetic construct; identical to SEQ ID NO: 191

SEQ ID NO: 374 - protein, 65 aa, synthetic construct; identical to SEQ ID NO: 192

SEQ ID NO: 375 - protein, 70 aa, synthetic construct; identical to SEQ ID NO: 193

SEQ ID NO: 376 - protein, 75 aa, synthetic construct; identical to SEQ ID NO: 194

SEQ ID NO: 377 - protein, 32 aa, synthetic construct; identical to SEQ ID NO: 195

SEQ ID NO: 378 - protein, 48 aa, synthetic construct; identical to SEQ ID NO: 196

SEQ ID NO: 379 - protein, 32 aa, synthetic construct; identical to SEQ ID NO: 195

SEQ ID NO: 380 - protein, 96 aa, synthetic construct
LEPGEKPYKCPECGKSFSQSGALTRHQRTHTRLEPGEKPYKCPECGKSFSQSGALTRHQRTHTRLEPGEKPYKCPECGKSFSQSGALTRHQRTHTR

SEQ ID NO: 381 - protein, 20 aa, synthetic construct
PKKKRKVEASSPKKRKVEAS

SEQ ID NO: 382 - DNA, 20 bp, Mus musculus
  source 1..20: mol_type = other DNA
aggtgaaggtgtggttccag

SEQ ID NO: 383 - DNA, 21 bp, Mus musculus
  source 1..21: mol_type = other DNA
tagtcattggaggtgacatca

SEQ ID NO: 384 - DNA, 20 bp, Homo sapiens
  source 1..20: mol_type = other DNA
aggtgaaggtgtggttccag

SEQ ID NO: 385 - DNA, 21 bp, Homo sapiens
  source 1..21: mol_type = other DNA
tagtcattggaggtgacatcg

SEQ ID NO: 386 - RNA, 20 nt, synthetic construct
  source 1..20: mol_type = other RNA
atggtgggaacccttcttct

SEQ ID NO: 387 - RNA, 20 nt, synthetic construct
  source 1..20: mol_type = other RNA
gtgatgggagcccttcttct

SEQ ID NO: 388 - DNA, 254 bp, synthetic construct
  source 1..254: mol_type = other DNA
gtaaaacgacggccagttaatacgactcactataaggataatggtggctgcgggaatctcagacaccttaaacgctcatggaggctatgaaaatggtctgcgaagtgagaatcacgcgactttagtcgtgtgaggttcaaatggtgggaacccttcttctggccggcatggtcccagcctcctcgctggcgccggctgggcaacatgcttcggcatggcgaatgggactgaagagcggtcatagctgtttcctg

SEQ ID NO: 389 - protein, 393 aa, Helicobacter pylori
MLITYKQKLYKNDKNRRIDTLLRRYGVFYNHCIALHKRYYRLFKKYLKLNDLQKHTTKLKKTHRYAFLKTLGSQTLQDLTERIDKAFKKFFKKQAKLPRFKKVANYKSFTFKSQADKKTGLNKGVGFEIKDNVVSFNGYSYKFIKTYALIGKVKTLTIKRDNMGDYFLCLVCELENNPNKQTACDKSVGFDFGLRTFLTGSDNTKIESPLFFSQYLPLIKRLSKNLSKKVKGSNNFKKAKKKLAQLHQKIKHLRTDFFYKLALKLSKKYQSIFIEDLNMKAMQKLWGRKVSDIAFSEFVKILENKAHVVKIDRFYPSSKTCSNCLSVDENFNKDIKKLGKTDKEREYHCKYCGLELDRDLNASINIHRVGASILGVEFVRPTSRADPKKKRKV

SEQ ID NO: 390 - DNA, 1182 bp, synthetic construct
  source 1..1182: mol_type = other DNA
atgctgatcacctacaagcagaagctgtacaagaacgacaagaacaggcggatcgacaccctgctgcggcggtacggcgtgttctacaaccactgtatcgccctgcacaagcggtactaccggctgttcaagaagtacttgaagctgaacgatctgcagaagcacaccaccaagctgaagaagacccacaggtacgccttcctgaagaccctgggcagccagaccctgcaggatctgacagagcggatcgacaaggccttcaagaagttcttcaagaagcaggccaagctgcccaggttcaagaaggtggccaactacaagagcttcaccttcaagtctcaggccgacaagaagaccggcctgaacaagggcgtgggcttcgagatcaaggacaatgtggtgagcttcaacggctacagctacaagttcatcaagacctacgccctgatcggcaaggtgaagaccttgaccatcaagcgggacaacatgggagactacttcctgtgtctggtgtgcgagctggagaacaatccaaacaagcagaccgcctgcgataagagcgtgggcttcgacttcggcctgagaaccttcctgaccggctccgacaacaccaagatcgagagccccctgttcttcagccagtatctgcctctgatcaagaggctgagcaagaacctgagcaagaaggtgaagggcagcaacaacttcaagaaggccaagaagaagctggcccagctgcaccagaagatcaagcacctgaggaccgacttcttctacaagctggccctgaagctgagcaagaagtaccagagcatcttcatcgaggacctgaacatgaaggccatgcagaagctgtggggcaggaaggtgagcgacatcgccttctccgagttcgtgaagatcctggagaacaaggcccacgtggtgaagatcgaccggttctaccccagcagcaagacctgcagcaactgcctgtccgtggacgagaacttcaacaaggacatcaagaagctggggaagaccgacaaggagcgggagtaccactgcaagtactgcggcctggagctggacagagacctgaacgccagcatcaacatccaccgcgtgggcgcctctatcctgggcgtggagttcgtgaggcccacctccagagccgaccccaagaagaagaggaaggtgtga

SEQ ID NO: 391 - RNA, 133 nt, synthetic construct
  source 1..133: mol_type = other RNA
attcatagggttggggcatcaatccttggggtagaatttgtaagacctacctagtaggctgagtttgcttgatcccaatttttctcatgctttagctagaatcccctagctttagctatggggagtatgtcaa

SEQ ID NO: 392 - protein, 416 aa, Deinococcus murrayi
MESTTAPKRNKAFVIRLYPNKAQAERINRTLGCARFVYNHFLARRIETYRQDGKGMTYAATDRALTLLKREEGTAWLAEVDKFALQQSLRDLERAYQNFFRTVKKSGKKVGFPKFKKKRTGEAYRTQFTNNNIEIGKGCIKLPKLGWVKTRGQRDMQGKILNVTVRRVHEGHYEASVLCEVEIPYLPAAPRFAAGVDVGIKDFAIVTDGEGEFTHHANPKYYRNGLKKLRKAQKTLSRRKKGSARYGKAKTKLARIHKRVANKRQDFIHKLTTALVREYEITCTEHLKPDNMRKNRRLALSISDAGWGEFIRQLEYKATWYGRLVSKISPHFPSSQMCHDCGSLNPAVKNLAVREWTCPNCGETHDRDENAALNIRREGLVAAGISDTQNARRECVSPAPAGNVRSRADPKKKRKV

SEQ ID NO: 393 - DNA, 1251 bp, synthetic construct
  source 1..1251: mol_type = other DNA
atggagagcaccaccgcccccaagaggaacaaggccttcgtgatccgcctgtaccccaacaaggcccaggccgagagaatcaatcggaccctgggctgcgccaggttcgtgtacaaccacttcctggccaggcggatcgagacctacaggcaggacggcaaagggatgacctatgccgccaccgatagggccctgaccctgctgaagagggaagagggcacagcttggctggccgaagtggacaagttcgccctgcagcagtccctgagggacctggagcgggcctaccagaacttcttccggactgtgaagaagtccggcaagaaggtgggcttccccaagttcaagaagaagcggactggcgaggcctacaggacccagttcaccaacaacaacatcgagatcggcaagggctgcatcaagctgcccaagctgggctgggtgaagaccaggggccagagggacatgcagggcaagatcctgaacgtgaccgtgcgcagagtgcacgagggccactatgaggccagcgtgctgtgtgaggtggagatcccttatctgcctgccgcccctagattcgccgccggcgtggatgtgggaatcaaggacttcgccatcgtgaccgatggcgagggcgagttcacccaccacgccaatcctaagtactacaggaacggcctgaagaagctgaggaaggctcagaagaccctgagccggcggaagaagggctccgccagatacggcaaggccaagaccaagctggcccgcattcacaagcgggtggccaacaagcggcaggacttcatccacaagctgaccacagccctggtgagggagtacgagatcacctgcaccgagcacctgaagcccgacaacatgcggaagaacaggagactggccctgagcatcagcgatgccggctggggcgagtttatcaggcagctggagtacaaggctacctggtacggcaggctggtgagcaagatcagcccccacttccccagcagccagatgtgtcacgactgcggcagcctgaaccccgccgtgaagaatctggccgtgagggagtggacctgccctaattgtggggagacccacgacagggacgagaacgccgccctgaacatcagaagagagggcctggtggccgccggcatctctgacacccagaatgccagaagagagtgtgtgagccctgcccctgccggcaatgtgagaagcagagccgatcctaagaagaagcggaaggtgtga

SEQ ID NO: 394 - RNA, 214 nt, synthetic construct
  source 1..214: mol_type = other RNA
gtgaagaatcttgccgtccgcgaatggacttgcccgaactgcggggaaacccatgaccgagacgagaacgccgcgctgaacatccggcgtgaaggactggtggccgcagggatttcggacacccaaaacgcccgtcgagaatgtgtaagcccagctccggctggcaacgttcgctgaagcgggaatcccgcgacttcagtcgtgggaggttcaa

SEQ ID NO: 395 - protein, 384 aa, Alicyclobacillus macrosporangiidus
MGRKRALIVLNDLCYNNGMNLTLMVKLLPTTEQHQALLETMERFNAACNAIAEVAFEHRTANKIRLQKLVYDSIRKEFGLSAQMTVRAIAKACEAYKRDKSIKPTFKPHGAIVYDQRLLSWKGLDRVSILTLGGRILVPILFGEYQAARLQRIRGQADLIYRDGTFYLAVVVDVPDPPQGAPNGFLGVDLGIKNIATDSDGEVFSGGHVNGLRHRHARLRQRLQSKGTKSAKRLLKKRRRKEARFATNVNHRIAKVLVAKAKDTGRGIALEDLKGIRDRITVRKAQRRTQHSWAFHQLRFFIEYKARLAGVPVVFVDPRNTSRTCPSCGHADKRNRPTRDNFECVECGFAGPADTIAAVNIRRRAEVMQPYAVSRADPKKKRKV

SEQ ID NO: 396 - DNA, 1155 bp, synthetic construct
  source 1..1155: mol_type = other DNA
atgggccggaagagggccctgattgtgctgaacgatctgtgctacaacaacggcatgaacctgaccctgatggtcaagctgctgcccaccacagagcagcatcaggccctgctggagaccatggagcggttcaatgccgcttgcaatgccattgccgaggtggccttcgagcacaggaccgccaacaagatccggctgcagaagctggtgtacgacagcatccgcaaggagttcggcctgtccgcccagatgactgtgagagccatcgccaaggcctgtgaggcctacaagagggacaagagcatcaagcctaccttcaagcctcacggcgccatcgtgtacgaccagagactgctgtcctggaagggcctggacagggtgagcatcctgaccctgggcggcagaatcctggtgcctatcctgtttggcgagtaccaggccgccagactgcagaggatcaggggccaggccgatctgatctaccgggatggcaccttctacctggccgtggtggtggatgtgccagatccacctcagggcgcccctaatggcttcctgggcgtggatctgggcatcaagaacatcgccaccgacagcgacggcgaggtgttctctggcggccacgtgaatggcctgaggcacagacatgccaggctgagacagagactgcagtctaagggcaccaagagcgccaagagactgctgaagaagagaagaaggaaggaggccaggttcgccaccaacgtgaaccaccggatcgccaaggtcctggtggccaaagccaaggataccggcagaggcattgccctggaggacctgaagggcatcagggacaggatcaccgtgagaaaggcccagcgcagaacccagcacagctgggcctttcaccagctgcggttcttcatcgagtacaaggccagactggccggcgtgcctgtggtgttcgtggaccccaggaacaccagcagaacctgcccttcttgcggccacgccgacaagaggaacagacccaccagggacaacttcgagtgcgtggagtgcggcttcgccggccctgccgatacaatcgccgccgtgaatatcaggagaagggccgaggtgatgcagccctacgccgtgagcagggccgaccctaagaagaagcgcaaggtgtga

SEQ ID NO: 397 - RNA, 146 nt, synthetic construct
  source 1..146: mol_type = other RNA
ttgagtgtgtcgaatgtgggttcgctggcccagccgacaccatcgctgcggtgaacattcgccgtagggctgaagtcatgcagccgtacgcggtatagcgcaggctacaactgcaagccccgccctttagggctggggttcatgac

SEQ ID NO: 398 - protein, 7 aa, synthetic construct
PKKKRKV

SEQ ID NO: 399 - protein, 8 aa, synthetic construct
VSRKRPRP

SEQ ID NO: 400 - protein, 6 aa, synthetic construct
QRKRKQ

SEQ ID NO: 401 - skipped

SEQ ID NO: 402 - RNA, 81 nt, synthetic construct
  source 1..81: mol_type = other RNA
ggaaatcgcaaaatttgctcttcgcgttagatttcttttagttttctcgcaactagcaagctttttgttctcgccgccgcc

SEQ ID NO: 403 - RNA, 81 nt, synthetic construct; identical to SEQ ID NO: 402
  source 1..81: mol_type = other RNA

SEQ ID NO: 404 - RNA, 81 nt, synthetic construct
  source 1..81: mol_type = other RNA
ggaaatcgcaaaattttcttttcgcgttagatttcttttagttttctttcaactagcaagctttttgttctcgccgccgcc

SEQ ID NO: 405 - RNA, 83 nt, synthetic construct
  source 1..83: mol_type = other RNA
ggaaatcgcaaaatttttgctctttttcgcgttagatttcttttagttttctykcaactagcaagctttttgttctcgccrcc

SEQ ID NO: 406 - RNA, 203 nt, synthetic construct
  source 1..203: mol_type = other RNA
ggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 407 - RNA, 76 nt, synthetic construct
  source 1..76: mol_type = other RNA
ggaaataagagagaaaagaagagtaagaagaaatataagatctccctgagcttcagggagccccggcgccgccacc

SEQ ID NO: 408 - RNA, 89 nt, synthetic construct
  source 1..89: mol_type = other RNA
ggaaaccccccacccccgtaagagagaaaagaagagtaagaagaaatataagatctccctgagcttcagggagccccggcgccgccacc

SEQ ID NO: 409 - RNA, 59 nt, synthetic construct
  source 1..59: mol_type = other RNA
ggagaacttccgcttccgttggcgcaagcgctttcattttttctgctaccgtgactaag

SEQ ID NO: 410 - RNA, 46 nt, synthetic construct
  source 1..46: mol_type = other RNA
ggaaataagagagaaaagaagagtaagaagaaatataagagccacc

SEQ ID NO: 411 - RNA, 56 nt, synthetic construct
  source 1..56: mol_type = other RNA
ggaaataagagagaaaagaagagtaagaagaaatataagaccccggcgccgccacc

SEQ ID NO: 412 - RNA, 79 nt, synthetic construct
  source 1..79: mol_type = other RNA
ggaaactttatttagtgttactttattttctgtttatttgtgtttcttcagtgggtttgttctaatttccttggccgcc

SEQ ID NO: 413 - RNA, 79 nt, synthetic construct
  source 1..79: mol_type = other RNA
ggaaaatctgtattaggttggcgtgttctttggtcggttgttagtattgttgttgattcgtttgtggtcggttgccgcc

SEQ ID NO: 414 - RNA, 79 nt, synthetic construct
  source 1..79: mol_type = other RNA
ggaaaattattaacatcttggtattctcgataaccattcgttggattttattgtattcgtagtttgggttcctgccgcc

SEQ ID NO: 415 - RNA, 79 nt, synthetic construct
  source 1..79: mol_type = other RNA
ggaaattattattatttctagctacaatttatcattgtattattttagctattcatcattatttacttggtgatcaaca

SEQ ID NO: 416 - RNA, 79 nt, synthetic construct
  source 1..79: mol_type = other RNA
ggaaataggttgttaaccaagttcaagcctaataagcttggattctggtgacttgcttcaccgttggcgggcaccgatc

SEQ ID NO: 417 - RNA, 74 nt, synthetic construct
  source 1..74: mol_type = other RNA
ggaaatcgtagagagtcgtacttagtacatatcgactatcggtggacaccatcaagattataaaccaggccaga

SEQ ID NO: 418 - RNA, 79 nt, synthetic construct
  source 1..79: mol_type = other RNA
ggaaacccgcccaagcgaccccaacatatcagcagttgcccaatcccaactcccaacacaatccccaagcaacgccgcc

SEQ ID NO: 419 - RNA, 79 nt, synthetic construct
  source 1..79: mol_type = other RNA
ggaaagcgattgaaggcgtcttttcaactactcgattaaggttgggtatcgtcgtgggacttggaaatttgttgtttcc

SEQ ID NO: 420 - RNA, 79 nt, synthetic construct
  source 1..79: mol_type = other RNA
ggaaactaatcgaaataaaagagccccgtactcttttatttctattaggttaggagccttagcatttgtatcttaggta

SEQ ID NO: 421 - RNA, 79 nt, synthetic construct
  source 1..79: mol_type = other RNA
ggaaatgtgatttccagcaacttcttttgaatatattgaattcctaattcaaagcgaacaaatctacaagccatatacc

SEQ ID NO: 422 - RNA, 74 nt, synthetic construct
  source 1..74: mol_type = other RNA
ggaaatcgtagagagtcgtacttacgtggtcgccattgcatagcgcgcgaaagcaacaggaacaagaacgcgcc

SEQ ID NO: 423 - RNA, 74 nt, synthetic construct
  source 1..74: mol_type = other RNA
ggaaatcgtagagagtcgtacttagaataaacagagtcgggtcgacttgtctctgatactacgacgtcacaatc

SEQ ID NO: 424 - RNA, 79 nt, synthetic construct
  source 1..79: mol_type = other RNA
ggaaaatttgccttcggagttgcgtatcctgaactgcccagcctcctgatatacaactgttccgcttattcgggccgcc

SEQ ID NO: 425 - RNA, 79 nt, synthetic construct
  source 1..79: mol_type = other RNA
ggaaatctgagcaggaatcctttgtgcattgaagactttagattcctctctgcggtagacgtgcacttataagtatttg

SEQ ID NO: 426 - RNA, 79 nt, synthetic construct
  source 1..79: mol_type = other RNA
ggaaagcgattgaaggcgtcttttcaactactcgattaaggttgggtatcgtcgtgggacttggaaatttgttgccacc

SEQ ID NO: 427 - RNA, 79 nt, synthetic construct
  source 1..79: mol_type = other RNA
ggaaaattttagcctggaacgttagataactgtcctgttgtctttatatacttggtccccaagtagtttgtcttccaaa

SEQ ID NO: 428 - RNA, 104 nt, synthetic construct
  source 1..104: mol_type = other RNA
ggaaatttttttttgatattataagagtttttttttgatattaagaaaatttttttttgatattagaagagtaagaagaaatataagaccccggcgccgccacc

SEQ ID NO: 429 - RNA, 57 nt, synthetic construct
  source 1..57: mol_type = other RNA
ggaaataagagagaaaagaagagtaagaagaaatataagagccaaaaaaaaaaaacc

SEQ ID NO: 430 - RNA, 76 nt, synthetic construct
  source 1..76: mol_type = other RNA
ggaaatctccctgagcttcagggagtaagagagaaaagaagagtaagaagaaatataagaccccggcgccgccacc

SEQ ID NO: 431 - RNA, 47 nt, synthetic construct
  source 1..47: mol_type = other RNA
gggaaataagagagaaaagaagagtaagaagaaatataagagccacc

SEQ ID NO: 432 - RNA, 47 nt, synthetic construct; identical to SEQ ID NO: 431
  source 1..47: mol_type = other RNA

SEQ ID NO: 433 - RNA, 47 nt, synthetic construct; identical to SEQ ID NO: 431
  source 1..47: mol_type = other RNA

SEQ ID NO: 434 - RNA, 47 nt, synthetic construct; identical to SEQ ID NO: 431
  source 1..47: mol_type = other RNA

SEQ ID NO: 435-440 - skipped (6 entries)

SEQ ID NO: 441 - RNA, 15 nt, synthetic construct
  source 1..15: mol_type = other RNA
taagcccctccgggg

SEQ ID NO: 442 - RNA, 15 nt, synthetic construct
  source 1..15: mol_type = other RNA
taaagctccccgggg

SEQ ID NO: 443-449 - skipped (7 entries)

SEQ ID NO: 450 - RNA, 1490 nt, synthetic construct
  source 1..1490: mol_type = other RNA
gggaaataagagagaaaagaagagtaagaagaaatataagaccccggcgccgccaccatgatccggaacaaggcttttgttgtccggctgtaccccaacgccgctcagaccgagctgatcaacagaacacttgggtctgccaggttcgtgtacaaccacttcctggcccgaagaatcgccgcatataaggaaagcggcaaaggcctgacctacggccagacaagcagcgagctgaccctgctgaaacaggccgaggaaaccagctggctgagcgaggtggataagttcgccctgcagaacagcctgaagaacctggaaaccgcctacaagaatttcttcagaacagtgaagcagagcggcaagaaagtgggatttcccagatttagaaagaagcggacaggcgagagctacaggacacagttcaccaacaacaacatccaaatcggcgagggcagactcaaactgcctaagctgggctgggtcaagacaaagggacagcaagacatccagggcaagatcctgaacgtgaccgtgcggagaatccatgaaggccactacgaggccagcgtgctgtgcgaggtggaaatcccctacctgcctgccgcccctaagttcgctgccggcgtggacgtgggcatcaaggacttcgccatcgtgaccgacggcgtgagattcaagcacgagcagaatcctaaatactacagaagcaccctgaagagactgagaaaagcccagcagaccctgtctagacggaaaaagggcagtgctagatacggcaaggccaagaccaagctggccagaatccacaagagaattgtgaacaagcgccaagatttcctgcacaagctgacaacaagcctggtgcgggaatacgagatcatcggcaccgagcacctgaagcctgacaacatgagaaaaaatcggagactcgccctgagcatcagcgacgccggctggggagaattcatcagacagctggagtacaaggccgcttggtatggcagactggtcagcaaggtgtccccttacttcccatccagccagctgtgtcacgattgcggctttaagaacccagaagtgaagaacctggccgtgcggacttggacctgtcctaactgcggcgagacccacgacagagatgagaacgccgctctgaacatcagacgggaagccctcgtggccgctggaatttctgacaccctgaatgcccacggcggctacgtgcgccccgcctctgccggaaatggactgcggagcgaaaaccacgccaccctggtggtgtccagagccgaccctaagaaaaaacggaaggttgagttcggctccggctaactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacat

SEQ ID NO: 451 - RNA, 251 nt, synthetic construct
  source 1..251: mol_type = other RNA
gattcaagaatcccgaagtgaagaatcttgccgtccgtacatggacttgcccgaactgtggggaaacccatgaccgagacgagaacgctgcgctgaacattcggcgtgaagcgttggtggctgcgggaatctcagacaccttaaacgctcatggaggctatgtcagacctgcttcggcgggcaatggtctgcgaagtgagaatcacgcgactttagtcgtgtgaggttcaagtgatgggagcccttcttct

SEQ ID NO: 452 - DNA, 2043 bp, synthetic construct
  source 1..2043: mol_type = other DNA
atgataaggaataaggctttcgtggtcaggctgtacccaaatgcggctcagactgaactgattaaccgcacgctgggtagcgcaaggttcgtctacaaccacttccttgcccgtcgcattgcggcctacaaggaaagcgggaagggactgacctacgggcaaacgagtagcgaactgacccttctgaagcaggctgaagaaacctcctggctctcggaagtagataagtttgctttgcagaactcgctgaaaaaccttgagaccgcgtacaagaacttctttcggactgtgaagcagtccggtaaaaaggtaggattcccacgtttcagaaagaagcgcacgggagagtcctaccggactcaattcaccaacaacaacatccaaattggggaaggtaggctcaaacttcctaagctgggatgggtgaaaaccaagggccagcaggatattcaagggaagattctgaatgtcactgtgcgccgtattcacgaaggccattacgaagcgtccgttctctgtgaagtcgagattccctacctgcctgcggctcccaagtttgcagcgggtgtggatgtcggcatcaaggattttgccatcgtgaccgatggcgtgaggtttaagcatgaacagaatccgaaatattaccgctccaccctgaaaagacttcgtaaagctcagcaaaccctgtccagacggaagaagggcagcgcacgttacgggaaagcgaaaaccaagctggctcggattcacaagcgcattgtcaataagcgtcaggatttccttcacaagctcaccacctccctggtgcgtgagtacgaaatcatcggaaccgaacaccttaaacccgacaacatgcggaaaaatcgccgccttgcactgagcatcagtgatgcgggctggggtgagttcatccggcagttggaatacaaggcagcgtggtacgggcgactggtatctaaagtcagcccctactttccatctagccagttgtgtcatgactgcggattcaagaatcccgaagtgaagaatcttgccgtccgtacatggacttgcccgaactgtggggaaacccatgaccgagacgagaacgctgcgctgaacattcggcgtgaagcgttggtggctgcgggaatctcagacaccttaaacgctcatggaggctatgtcagacctgcttcggcgggcaatggtctgcgaagtgagaatcacgcgactttagtcgtgagcagggccgacccaaagaagaagcggaaggtcgaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 453 - protein, 426 aa, Geobacillus stearothermophilus
MYFCIKQQLNGLTKEEYLTLRELCHIAKNMYNVGLYNVRQYYFEHKEFLNYEKNYHLAKTNENYKLLNSNMAQQILKKVNEAFKSFFGLISLAKQGKYDHKAISIPKYLKKDGFHSLIIGQIRIDGNKFTIPYSRLFKKTHKPITITIPPVLLDKKIKQIEIIPKHHARFFEIQYKYEMPEDQRELNDQKALAIDLGLNNLATCVTSDGRSFIIDGRRLKSINQWFNKENARLQSIKDKQKIKGTTRKQALLAMNRNNKVNDYINKTCRYIINYCIENQIGKLVIGYAETWQRNMNLGKKTNQNFVNIPLGNIKEKLEYLCEFYGIEFLKQEESYTSQASFFDGDEIPEYNADNPKEYKFSGKRIKRGLYRTKSGKLINADVNGALNILKKSKAVDLSVLCSSGEVDTPQRIRIASRADPKKKRKV

SEQ ID NO: 454 - DNA, 1281 bp, synthetic construct
  source 1..1281: mol_type = other DNA
atgtacttctgcatcaagcagcagctgaacggcctgaccaaggaggagtatctgaccctgagggagctgtgtcacatcgccaagaacatgtacaacgtgggcctgtacaatgtgaggcagtactactttgagcacaaggagttcctgaactacgagaagaactaccacctcgccaagaccaacgagaactacaagctgctgaacagcaacatggcccagcagatcctgaagaaggtgaacgaggccttcaagtccttcttcggcctgatctccctggccaagcagggcaagtacgatcacaaggccatcagcatccccaagtacctgaagaaggacggcttccacagcctgatcatcggccagatcaggatcgacggcaacaagttcaccatcccctacagccgcctgttcaagaagacccacaagcccatcaccatcaccatcccccccgtgctgcttgataagaagatcaagcagatcgagatcatccccaagcaccacgccagattcttcgagatccagtacaagtacgagatgccggaggatcagcgggagctgaatgatcagaaggccctggccatcgacctgggcctgaacaatctggccacatgcgtgaccagcgatggcagaagcttcatcatcgacggcaggcggctgaagtccatcaatcagtggttcaacaaggagaacgcccggctgcagtccatcaaggacaagcagaagatcaagggcaccacccggaagcaggccctgctggccatgaacaggaacaacaaggtgaacgactacatcaacaagacctgcaggtacatcatcaactactgcatcgagaaccagatcggcaagctggtgatcggctatgccgagacatggcagagaaacatgaatctcggcaagaagaccaaccagaacttcgtgaacatccccctggggaacatcaaggagaagctggagtacctgtgcgagttctacggcatcgagttcctgaagcaggaggagagctacacaagccaggcctccttcttcgacggggatgagatccccgagtacaacgccgacaaccccaaggagtacaagttcagcggcaagcgcattaagcgcgggctgtaccggaccaagtccggcaagctgatcaacgccgacgtgaacggcgccctgaacatcctgaagaagtccaaggccgtggacctgagcgtgctgtgtagcagcggcgaggtggatacccctcagaggatcagaatcgcctccagagccgaccccaagaagaagcggaaggtctga

SEQ ID NO: 455 - RNA, 263 nt, synthetic construct
  source 1..263: mol_type = other RNA
ttaagcgcggcttgtatcgaacaaagtctggcaaactaattaatgctgatgtcaatggcgcattaaacatcttaaagaaaagtaaagctgtagacctgagtgtcttatgctctagcggcgaagtggacacgcctcaaagaataaggattgcttgaagcagtcaaacttctttggaagcccccacttcaaattttcgctagaaaattaagtgggggtagttcacggacaagtggttcaccatgcgttgctttatggtatgatag

SEQ ID NO: 456 - DNA, 35 bp, synthetic construct
  source 1..35: mol_type = other DNA
ttgatgtgatgggagcccttcttcttctgctcgga

SEQ ID NO: 457 - DNA, 35 bp, synthetic construct
  source 1..35: mol_type = other DNA
tccgagcagaagaagaagggctcccatcacatcaa

SEQ ID NO: 458 - DNA, 32 bp, synthetic construct
  source 1..32: mol_type = other DNA
ttgatgtgatgggagcccttcttctgctcgga

SEQ ID NO: 459 - DNA, 29 bp, synthetic construct
  source 1..29: mol_type = other DNA
ttgatgtgatgggagcccttctgctcgga

SEQ ID NO: 460 - DNA, 35 bp, synthetic construct
  source 1..35: mol_type = other DNA
ttgatatggtgggaacccttcttcttctgctcaga

SEQ ID NO: 461 - DNA, 35 bp, synthetic construct
  source 1..35: mol_type = other DNA
tctgagcagaagaagaagggttcccaccatatcaa

SEQ ID NO: 462 - DNA, 32 bp, synthetic construct
  source 1..32: mol_type = other DNA
ttgatatggtgggaacccttcttctgctcaga

SEQ ID NO: 463 - DNA, 34 bp, synthetic construct
  source 1..34: mol_type = other DNA
ttgatatggtgggaaccctcttcttctgctcaga

SEQ ID NO: 464 - DNA, 11 bp, synthetic construct
  source 1..11: mol_type = other DNA
gtcaatggacc

SEQ ID NO: 465 - DNA, 11 bp, synthetic construct
  source 1..11: mol_type = other DNA
ggtccattgac

SEQ ID NO: 466 - protein, 6 aa, synthetic construct
HHHHHH

(19) United States
(12) Patent Application Publication
(10) Pub. No.: US 2025/0367322 A1
(43) Pub. Date: Dec. 4, 2025

(54) NUCLEOBASE EDITING SYSTEM AND METHOD OF USING SAME FOR MODIFYING NUCLEIC ACID SEQUENCES

(71) Applicant: ReNAgade Therapeutics Management, Inc., Cambridge, MA (US)

(72) Inventors: Alim LADHA, Cambridge, MA (US); Muthusamy JAYARAMAN, Cambridge, MA (US); Ganapathy Subramanian SANKARAN, Cambridge, MA (US)

(21) Appl. No.: 18/873,106

(22) PCT Filed: Jun. 9, 2023
(86) PCT No.: PCT/US2023/068233

§ 371 Date: Dec. 9, 2024

Related U.S. Application Data
(60) Provisional application No. 63/351,326, filed on Jun. 10, 2022.
(60) Provisional application No. 63/452,316, filed on Mar. 15, 2023.

(51) Int. Cl. A61K 48/00; A61K 9/51; A61K 31/7105; A61K 38/46; C12N 9/22; C12N 15/11; C12N 15/88; C12N 15/90

(52) U.S. Cl. CPC A61K 48/0033; A61K 9/5123; A61K 31/7105; A61K 38/465; C12N 9/22; C12N 15/11; C12N 15/88; C12N 15/907; C12N 2310/20

(57) ABSTRACT

The disclosure provides nucleic acid-containing lipid nanoparticle (LNP) compositions and methods relating to the delivery of TnpB nucleobase editing systems comprising TnpB polypeptides, engineered TnpB ncRNAs, and optionally one or more additional accessory functionalities (e.g., a deaminase, reverse transcriptase, recombinase, nuclease, a donor template, or combinations thereof) for use in applications such as precision gene editing.

### RELATED APPLICATIONS

This application claims priority to U.S. Provisional Application Ser. No. U.S. Provisional Application Ser. No. 63/351,326, filed Jun. 10, 2022 (Attorney Docket No. RNG018-P1) and U.S. Provisional Application Ser. No. 63/452,316, filed Mar. 15, 2023 (Attorney Docket No. RNG012-P2), each of which are incorporated herein by reference in their entireties. The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### TECHNICAL FIELD

The present disclosure generally relates to the field of nucleic acid-containing lipid nanoparticle (LNP) compositions and uses thereof in the delivery of TnpB nucleobase editing systems comprising TnpB polypeptides, engineered TnpB ncRNAs, and optionally one or more additional accessory functionalities (e.g., a deaminase, reverse transcriptase, recombinase, nuclease, a donor template, or combinations thereof) for use in applications such as precision gene editing. The disclosure further relates to methods of precise editing comprising administering an effective amount of an LNP-based TnpB nucleobase editing system comprising one or more nucleic acid and/or protein components for applications including precision gene editing under in vitro, ex vivo, and in vivo conditions. In various aspects, the LNPs may include coding RNA (e.g., linear and/or circular mRNAs) that encoding one or more polypeptide or nucleic acid components of the TnpB nucleobase editing system (e.g., TnpB polypeptide and/or one or more accessory proteins, such as a deaminase or reverse transcriptase and/or a donor template), and/or non-coding RNA (e.g., TnpB ncRNAs).

### BACKGROUND

The emergence of highly versatile genome-editing technologies—accelerated in the last decade largely by CRISPR/Cas9—has provided investigators with the ability to rapidly and economically introduce sequence-specific modifications into the genomes of a broad spectrum of cell types and organisms, paving the way for the appearance of a multitude of gene editing companies with clinical pipelines for gene editing medicines to treat a myriad of genetic disorders and complex diseases. The core gene editing technologies most commonly used to facilitate genome editing are clustered regularly interspaced short palindromic repeats (CRISPR)-CRISPR-associated nucleases (e.g., Class 2, Type II enzymes (e.g., Cas9) or Class 2, Type V enzymes (e.g., Cas12a)), transcription activator-like effector nucleases (TALENs), zinc-finger nucleases (ZFNs), and homing endonucleases or meganucleases.

While such genome-editing applications—such as targeted gene inactivation and precision editing—have been developed based on these technologies, there remains a need for new genome engineering technologies that employ novel strategies and molecular systems which have higher efficiency, better deliverability (particularly in vivo), improved precision editing, and which remain affordable, easy to scale and manufacture, and which have improved targeting ability within the genome.

In a recent publication, Karvelis et al., “Transposon-associated TnpB is a programmable RNA-guided DNA endonuclease,” Nature, Nov. 25, 2021, Vol. 599, pp. 692-700 (which is incorporated herein by reference), the authors elucidated the function of the TnpB protein demonstrating that TnpB of Deinococcus radiodurans ISDra2 is an RNA-directed nuclease that is guided by right-end (RE) derived RNA (“reRNA”) to cleave DNA next to 5′ TTGAT transposon associated motif (TAM). Karvelis et al. also reported on the use of TnpB as a genome editor to cleave DNA target sites in a human cell line HEK293T.

However, there remains much room for improvement and design to achieve an effective TnpB-based gene editing system having sufficient editing efficiency, improved precision, better deliverability, and which remains affordable, easy to scale, and has improved ability to treat various genetic disorders and complex diseases. An improved TnpB-based gene editing system would be a significant advance in the art.

### SUMMARY

The present disclosure provides TnpB-based genome editing systems for use in various applications, including precision gene editing in cells, tissues, organs, or organisms. In addition, the disclosure provides LNP compositions comprising said TnpB-based genome editing systems for use in various applications, including precision gene editing in cells, tissues, organs, or organisms. In various embodiments, the TnpB-based genome editing systems comprise (a) a TnpB polypeptide (or a nucleic acid molecule encoding same) and (b) a recombinant TnpB ncRNA (comprising a guide RNA)(or a nucleic acid molecule encoding same) which is capable of associating with the TnpB polypeptide to form a complex such that the complex localizes to a target nucleic acid sequence (e.g., a genomic or plasmid target sequence) and binds thereto. In various embodiments, the TnpB protein has a nuclease activity which results in the cutting of one or both strands of DNA. In various embodiments, the TnpB polypeptide is a polypeptide selected from Table A, or a polypeptide having at least 70%, 75%, 80%, 85%, 90%, 95%, 99%, or 100% sequence identity with a polypeptide from Table A. In various other embodiments, exemplary TnpB ncRNAs are provided in Table B, or a nucleic acid molecule having at least 70%, 75%, 80%, 85%, 90%, 95%, 99%, or 100% sequence identity with a TnpB ncRNA sequence of Table B. In addition, the disclosure contemplates any suitable TnpB ncRNA that may be obtained and/or engineered by known methods as referenced in the herein disclosure and in the Examples.

In various embodiments, the TnpB ncRNA may comprise (a) a region that binds or associates with a TnpB protein and (b) a region that comprises a targeting or “guide” sequence, i.e., a sequence which is complementary to a target nucleic acid sequence.

In another aspect, the compositions comprising the TnpB-based genome editing systems may comprise one or more additional accessory proteins (or nucleic acid molecules encoding same) having genome modifying functions, including recombinases, invertases, nucleases, polymerases, ligases, deaminases, or reverse transcriptases. In various embodiments, the accessory proteins may be encoded separate from the TnpB protein. In other embodiments, the accessory proteins may be fused to TnpB, optionally with a linker.

In still another aspect, the disclosure provides delivery systems (e.g., LNP delivery systems) for introducing the TnpB-based genome editing systems and/or components thereof into cells, tissues, organs, or organisms. Depending on the chosen format, the TnpB genome editing systems and/or the individual or combined components thereof may be delivered as DNA molecules (e.g., encoded on one or more plasmids), non-coding RNA molecules (e.g., reRNAs for targeting the TnpB protein), coding RNA molecules (e.g., linear or circular mRNAs coding for the TnpB protein and/or accessory protein components of the TnpB systems), proteins (e.g., TnpB polypeptides, accessory proteins having other functions (e.g., recombinases, nucleases, polymerases, ligases, deaminases, or reverse transcriptases), or protein-nucleic acid complexes (e.g., complexes between an reRNA and a TnpB protein or fusion protein comprising a TnpB protein).

In another aspect, the present disclosure provides nucleic acid molecules encoding the TnpB-based genome editing systems or components thereof. In yet another aspect, the disclosure provides vectors for transferring and/or expressing said TnpB-based genome editing systems, e.g., under in vitro, ex vivo, and in vivo conditions. In still another aspect, the disclosure provides cell-delivery compositions and methods, including compositions for passive and/or active transport to cells (e.g., plasmids), delivery by virus-based recombinant vectors (e.g., AAV and/or lentivirus vectors), delivery by non-virus-based systems (e.g., liposomes and LNPs), and delivery by virus-like particles. Depending on the delivery system employed, the TnpB-based genome editing systems described herein may be delivered in the form of DNA (e.g., plasmids or DNA-based virus vectors), RNA (e.g., reRNA and mRNA delivered by LNPs), a mixture of DNA and RNA, protein (e.g., virus-like particles), and ribonucleoprotein (RNP) complexes. Any suitable combinations of approaches for delivering the components of the herein disclosed TnpB-based genome editing systems may be employed. In a preferred embodiment, the TnpB nucleobase editing systems are delivered by way of LNP compositions.

In other embodiments, the TnpB-based genome editing systems may comprise a template DNA comprising an edit, e.g., a single strand or double strand donor molecule (linear or circular) which may be used by the cell to repair a single or double cut lesion introduced by a TnpB-reRNA complex.

In one embodiment, each of the components of the TnpB-based genome editing systems is delivered by an all-RNA system, e.g., the delivery of one or more RNA molecules (e.g., mRNA and/or reRNA) by one or more LNPs, wherein the one or more RNA molecules form the reRNA and guide RNA (as needed) and/or are translated into the polypeptide components (e.g., the TnpB and an accessory protein), and a DNA or RNA-encoded template DNA molecule (e.g., donor template).

In yet another aspect, the disclosure provides methods for genome editing by introducing a TnpB-based genome editing system described herein into a cell (e.g., under in vitro, in vivo, or ex vivo conditions) comprising a target edit site, thereby resulting in an edit at the target edit. In other aspects, the disclosure provides formulations comprising any of the aforementioned components for delivery to cells and/or tissues, including in vitro, in vivo, and ex vivo delivery, recombinant cells and/or tissues modified by the recombinant TnpB-based genome modification systems and methods described herein, and methods of modifying cells by conducting genome editing using the herein disclosed TnpB-based genome editing systems.

The disclosure also provides methods of making the TnpB-based genome editing system, their protein and nucleic acid molecule components, vectors, compositions and formulations described herein (e.g., LNP compositions), as well as to pharmaceutical compositions and kits for modifying cells under in vitro, in vivo, and ex vivo conditions that comprise the herein disclosed genome editing and/or modification systems.

In various embodiments, the disclosure relates to the following numbered paragraphs:

1 A pharmaceutical composition comprising:
a) at least one lipid nanoparticle (LNP) comprising at least one ionizable lipid selected from those listed in Tables (I), (II), (III), (IV) or (V); and
b) at least one TnpB gene editing system.

2. The pharmaceutical composition of paragraph 1, wherein the ionizable lipid is from Table (I).

3. The pharmaceutical composition of paragraph 1, wherein the ionizable lipid is from Table (II).

4. The pharmaceutical composition of paragraph 1, wherein the ionizable lipid is from Table (III).

5. The pharmaceutical composition of paragraph 1, wherein the ionizable lipid is from Table (IV).

6. The pharmaceutical composition of paragraph 1, wherein the ionizable lipid is from Table (V).

7. The pharmaceutical composition of paragraph 1, wherein the at least one TnpB gene editing system is capable of editing, modifying or altering a polynucleotide sequence.

8. The pharmaceutical composition of paragraph 1, wherein the at least one TnpB gene editing system comprises:
a) a nucleic acid sequence encoding a TnpB protein or functional variant thereof;
b) a TnpB ncRNA or a nucleic acid sequence encoding same, wherein the ncRNA comprises an engineered guide.

9. The pharmaceutical composition of paragraph 8, wherein the TnpB protein is selected from any TnpB protein of Table A or functional fragment thereof, or an amino acid sequence having at least 85%, 90%, 95%, 99%, or up to 100% sequence identity with any of the TnpB proteins of Table A.

10. The pharmaceutical composition of paragraph 8, wherein the TnpB ncRNA is selected from any nucleic acid sequence from Table B or functional fragment thereof, or a nucleic acid sequence having at least 85%, 90%, 95%, 99%, or up to 100% sequence identity with any nucleic acid sequence from Table B.

11. The pharmaceutical composition of paragraph 8, wherein component a) is a coding RNA and b) is a TnpB ncRNA.

12. The pharmaceutical composition of paragraph 8, wherein the coding RNA is a linear mRNA or a circular mRNA.

13. The pharmaceutical composition of paragraph 8, wherein the TnpB gene editing system further comprises a donor DNA template capable of modifying a target sequence.

14. The pharmaceutical composition of paragraph 13, wherein the donor DNA template is double-stranded DNA.

15. The pharmaceutical composition of paragraph 13, wherein the donor DNA template is single-stranded DNA.

16. The pharmaceutical composition of paragraph 13, wherein the donor DNA template is circular single-stranded DNA.

17. The pharmaceutical composition of paragraph 13, wherein the donor DNA template comprises an edit flanked by regions of homology to the regions upstream and downstream of a TnpB cut site.

18. The pharmaceutical composition of paragraph 1, wherein the TnpB editing system is capable of installing an edit at a target site.

19. The pharmaceutical composition of paragraph 18, wherein the edit comprises a double-strand cut.

20. The pharmaceutical composition of paragraph 18, wherein the edit comprises an insertion of 1 or more nucleobases, a deletion of 1 or more nucleobases, or a combination thereof.

21. The pharmaceutical composition of paragraph 18, wherein the edit is a transversion edit.

22. The pharmaceutical composition of paragraph 18, wherein the edit is a transition edit.

23. The pharmaceutical composition of paragraph 18, wherein the edit converts a T←→Cor A←␣G.

24. The pharmaceutical composition of paragraph 18, wherein the edit converts a T→A or G, C→G or A, A→T or C, or G→C or T.

25. The pharmaceutical composition of paragraph 20, wherein the insertion or deletion is of a whole exon or intron of a gene.

26. The pharmaceutical composition of paragraph 20, wherein the insertion or deletion is of a whole or partial gene.

27. The pharmaceutical composition of paragraph 1, wherein the TnpB gene editing system further comprises an accessory protein or a nucleotide sequence encoding the accessory protein.

28. The pharmaceutical composition of paragraph 27, wherein the accessory protein is selected from the group consisting of a nuclease, a deaminase, a recombinase, a reverse transcriptase, and an integrase.

29. The pharmaceutical composition of paragraph 27, wherein the accessory protein is fused to a TnpB protein to form a fusion protein.

30. The pharmaceutical composition of paragraph 29, wherein the fusion protein comprises a TnpB protein and a deaminase.

31. The pharmaceutical composition of paragraph 29, wherein the fusion protein comprises a TnpB protein and a reverse transcriptase.

32. The pharmaceutical composition of paragraph 29, wherein the fusion protein comprises a TnpB protein and a recombinase.

33. The pharmaceutical composition of paragraph 29, wherein the fusion protein comprises a TnpB protein and a nuclease.

34. The pharmaceutical composition of paragraph 29, wherein the fusion protein comprises a TnpB protein and an integrase.

35. The pharmaceutical composition of any of the above paragraphs for ex vivo delivery.

36. The pharmaceutical composition of any of the above paragraphs for in vivo delivery.

37. The pharmaceutical composition of any of the above paragraphs wherein the TnpB gene editing system recognizes a transposon-associated motif (TAM).

38. The pharmaceutical composition of any of the above paragraphs wherein the TnpB gene editing system treats one or more monogenic disorders or diseases.

39. The pharmaceutical composition of paragraph 8, wherein the TnpB ncRNA comprises one or more chemical modifications selected from 2′-O-Me, 2′-F, and 2′F-ANA at 2′OH; 2′F-4′-Cα-OMe and 2′,4′-di-Cα-OMe at 2′ and 4′ carbons; phosphodiester modifications comprising sulfide-based Phosphorothioate (PS) or acetate-based phosphonoacetate alterations; combinations of the ribose and phosphodiester modifications; locked nucleic acid (LNA), bridged nucleic acids (BNA), S-constrained ethyl (cEt), and unlocked nucleic acid (UNA); modifications to produce a phosphodiester bond between the 2′ and 5′ carbons (2′,5′-RNA) of adjacent RNAs; and a butane 4-carbon chain link between adjacent RNAs.

40. A method for editing the DNA of a host cell comprising delivering an effective amount of a pharmaceutical composition of any of the above paragraphs.

41. A method for editing a target sequence in the DNA of a host cell comprising delivering an effective amount of a pharmaceutical composition comprising at least one lipid nanoparticle (LNP) comprising at least one ionizable lipid selected from those listed in Tables (I), (II), (III), (IV) or (V); and at least one TnpB gene editing system, wherein the TnpB gene editing system comprises a nucleic acid sequence encoding a TnpB protein or functional variant thereof; and a TnpB ncRNA or a nucleic acid sequence encoding same, thereby installing an edit to the target sequence.

42. The method for editing of paragraph 41, wherein the ionizable lipid is from Table (I).

43. The method for editing of paragraph 41, wherein the ionizable lipid is from Table (II).

44. The method for editing of paragraph 41, wherein the ionizable lipid is from Table (III).

45. The method for editing of paragraph 41, wherein the ionizable lipid is from Table (IV).

46. The method for editing of paragraph 41, wherein the ionizable lipid is from Table (V).

47. The method for editing of paragraph 41, wherein the TnpB gene editing system is capable of editing, modifying or altering the target sequence.

48. The method for editing of paragraph 41, wherein the TnpB protein is selected from any TnpB protein of Table A or functional fragment thereof, or an amino acid sequence having at least 85%, 90%, 95%, 99%, or up to 100% sequence identity with any of Table A TnpB proteins or functional fragment thereof.

49. The method for editing of paragraph 41, wherein the nucleic acid sequence encoding a TnpB protein is selected from any nucleic acid sequence from Table B or functional fragment thereof, or a nucleic acid sequence having at least 85%, 90%, 95%, 99%, or up to 100% sequence identity with any TnpB protein of Table A.

50. The method for editing of paragraph 41, wherein the nucleic acid sequence encoding the TnpB protein is a linear or circular mRNA.

51. The method for editing of paragraph 41, wherein the TnpB gene editing system further comprises a donor DNA template.

52. The method for editing of paragraph 51, wherein the donor DNA template is single-stranded or double-stranded DNA.

53. The method for editing of paragraph 51, wherein the donor DNA template is circular single-stranded DNA.

54. The method for editing of paragraph 51, wherein the donor DNA template comprises an edit flanked by regions of homology to the regions upstream and downstream of a TnpB cut site.

55. The method for editing of paragraph 41, wherein the edit comprises a double-strand cut 56 The method for editing of paragraph 41, wherein the edit comprises an insertion of 1 or more nucleobases, a deletion of 1 or more nucleobases, or a combination thereof.

57. The method for editing of paragraph 41, wherein the edit is a transversion edit.

58. The method for editing of paragraph 41, wherein the edit is a transition edit.

59. The method for editing of paragraph 41, wherein the edit converts a T←→C or A←→G.

60. The method for editing of paragraph 41, wherein the edit converts a T→A or G, C →G or A, A→T or C, or G→C or T.

61. The method for editing of paragraph 56, wherein the insertion or deletion is of a whole exon or intron of a gene.

62. The method for editing of paragraph 56, wherein the insertion or deletion is of a whole or partial gene.

63. The method for editing of paragraph 41, wherein the TnpB gene editing system further comprises an accessory protein or a nucleotide sequence encoding the accessory protein.

64. The method for editing of paragraph 63, wherein the accessory protein is selected from the group consisting of a nuclease, a deaminase, a recombinase, a reverse transcriptase, and an integrase.

65 The method for editing of paragraph 63, wherein the accessory protein is fused to a TnpB protein to form a fusion protein.

66. The method for editing of paragraph 65, wherein the fusion protein comprises a TnpB protein and a deaminase.

67. The method for editing of paragraph 65, wherein the fusion protein comprises a TnpB protein and a reverse transcriptase.

68. The method for editing of paragraph 65, wherein the fusion protein comprises a TnpB protein and a recombinase.

69. The method for editing of paragraph 65, wherein the fusion protein comprises a TnpB protein and a nuclease.

70. The method for editing of paragraph 65, wherein the fusion protein comprises a TnpB protein and an integrase.

71. The method for editing of paragraph 41 for ex vivo or in vivo delivery.

72. The method for editing of paragraph 41, wherein the TnpB gene editing system recognizes a transposon-associated motif (TAM).

73 The method for editing of paragraph 41, wherein the TnpB gene editing system treats one or more monogenic disorders or diseases.

In various other embodiments, the disclosure relates to the following numbered paragraphs:

1. A genome editing system comprising:
a. a nucleic acid sequence encoding an engineered TnpB protein;
b. a second nucleic acid sequence encoding a recombinant reRNA comprising a truncated reRNA selected from any one of the truncated reRNA sequences of Table D (SEQ ID NOs: 38838-77066), Table E (SEQ ID NOs: 77067-115495), or Table F (SEQ ID Nos: 115496-153924) and a guide RNA;
wherein the TnpB protein and the recombinant reRNA form a RNA-protein complex;
wherein the genome editing system optionally further comprises a donor nucleic acid sequence capable of modifying a target sequence; and
wherein the TnpB sequence is optionally a corresponding polypeptide from Table C(SEQ ID Nos: 209-38637).

2. The genome editing system of paragraph 1 wherein the nucleic acid sequence encoding the engineered TnpB protein is operably fused to one or more nucleic acid sequences encoding an endonuclease.

3. The genome editing system of paragraphs 1 or 2 wherein the nucleic acid sequence encoding the engineered TnpB protein is operably fused to one or more nucleic acid sequences encoding a deaminase.

4. The genome editing system of any one of the above paragraphs wherein the nucleic acid sequence encoding the engineered TnpB protein is operably fused to one or more nucleic acid sequences encoding a reverse transcriptase.

5. The genome editing system of any one of the above paragraphs wherein the nucleic acid sequence encoding the engineered TnpB protein is operably fused to one or more nucleic acid sequences encoding transcriptional modulating a polypeptide.

6. The genome editing system of any one of the above paragraphs wherein the nucleic acid sequence encoding the engineered TnpB protein comprises enhanced genome editing efficiency.

7. The genome editing system of any one of the above paragraphs wherein the enhanced genome editing efficiency comprises at least two to fivefold increase in editing efficiency relative to SpCas9.

8. The genome editing system of any one of the above paragraphs wherein TnpB sequence is a corresponding polypeptide from Table C(SEQ ID Nos: 209-38637).

9. The genome editing system of any one of the above paragraphs wherein the donor nucleic acid sequence repairs the target region of the genome editing system genome cleaved by the RNA-protein complex.

10. The genome editing system of any one of the above paragraphs wherein the nucleic acid sequence encoding the TnpB and the recombinant reRNA are transiently expressed in the host cell genome.

11. The genome editing system of any one of the above paragraphs wherein the nucleic acid sequence encoding the TnpB and the recombinant reRNA are integrated into the host cell genome.

12. The genome editing system of any one of the above paragraphs wherein the nucleic acid sequences encoding TnpB and the recombinant reRNA are integrated into a chromosome or a plasmid of the host cell genome.

13. The genome editing system of any one of the above paragraphs wherein the genome editing system comprises a second donor nucleic acid sequence paired with a one or more guide RNAs to modify a second target region of the host cell genome.

14. The genome editing system of any one of the above paragraphs wherein the host cell comprises an insertion or a stable integration of the one or more desired modification sequence into the host cell genome.

15. The genome editing system of any one of the above paragraphs wherein the donor nucleic acid sequence provides a modification to the target region of the host cell genome.

16. The genome editing system of any one of the above paragraphs wherein the modification of the target region comprises an insertion, deletion or alteration of one or more base pairs at the target region in the host cell genome.

17. The genome editing system of any one of the above paragraphs wherein the TnpB protein recognizes a transposon-associated motif (TAM).

18. The genome editing system of any one of the above paragraphs wherein the one or more desired modification sequence is selected from one or more sequences associated with one or more monogenic disorders or diseases.

19. The genome editing system of any one of the above paragraphs wherein the nucleic acid sequences encoding TnpB encode a protein selected from SEQ ID NO: 1-135.

20. The genome editing system of any one of the above paragraphs wherein the TnpB sequence comprises an amino acid sequence of any of SEQ ID Nos: 209-38637.

21. The genome editing system of any one of the above paragraphs wherein the nucleic acid sequence encoding TnpB is characterized as type-V CRISPR nuclease.

22. The genome editing system of any one of the above paragraphs wherein the TnpB comprises about 400-700 AA residues.

23. The genome editing system of any one of the above paragraphs wherein the TnpB comprises modifications in one or more domains selected from REC, WED, RuvC, HH and ZnF.

24. The genome editing system of any one of the above paragraphs wherein the TnpB comprises comprises at least 30%, 40%, 50%, 60%, 70%, 80%, 90%, 95%, 96%, 97%, 98%, 99%, 99.5% or higher sequence identity to a protein selected from SEQ ID NO: 1-135.

25. The genome editing system of any one of the above paragraphs further comprising a delivery vector.

26. The genome editing system of paragraph 25 wherein the delivery vector is selected from viral vector is selected from a retroviral vector, a lentiviral vector, an adenoviral, an adeno-associated viral vector, vaccinia viral vector, poxviral vector, and herpes simplex viral vector.

27. The genome editing system of paragraph 25 wherein the delivery vector comprises a non-viral vectors selected from cationic liposomes, lipid nanoparticles (LNPs), cationic polymers, vesicles, and gold nanoparticles.

28. The genome editing system of paragraph 25 wherein the genome editing system comprises enhanced transduction efficiency and/or low cytotoxicity.

29. The genome editing system of any one of the above paragraphs wherein the modification of the target sequence of the host cell genome comprises binding activity, cleavage activity, nickase activity, transcriptional activation activity, transcriptional inhibitory activity, or transcriptional epigenetic activity.

30. The genome editing system of any one of the above paragraphs wherein the recombinant reRNA comprises one or more chemical modifications selected from 2′-O-Me, 2′-F, and 2′F-ANA at 2′OH; 2′F-4′-Cα-OMe and 2′,4′-di-Cα-OMe at 2′ and 4′ carbons; phosphodiester modifications comprising sulfide-based Phosphorothioate (PS) or acetate-based phosphonoacetate alterations; combinations of the ribose and phosphodiester modifications; locked nucleic acid (LNA), bridged nucleic acids (BNA), S-constrained ethyl (cEt), and unlocked nucleic acid (UNA); modifications to produce a phosphodiester bond between the 2′ and 5′ carbons (2′,5′-RNA) of adjacent RNAs; and a butane 4-carbon chain link between adjacent RNAs.

31. A method for editing the DNA of a host cell,
a) producing one or more compositions comprising:
1. a nucleic acid sequence encoding an engineered TnpB protein;
2. a second nucleic acid sequence encoding a second nucleic acid sequence encoding a recombinant reRNA comprising a truncated reRNA selected from any one of the truncated reRNA sequences of Table D (SEQ ID NOs: 38838-77066), Table E (SEQ ID NOs: 77067-115495), or Table F (SEQ ID Nos: 115496-153924) and a guide RNA wherein the TnpB protein and the second nucleic acid sequence form a RNA-protein complex;
wherein the TnpB protein and the recombinant reRNA form a RNA-protein complex;
wherein the genome editing system optionally further comprises a donor nucleic acid sequence capable of modifying a target sequence; and
wherein the TnpB sequence is optionally a corresponding polypeptide from Table C(SEQ ID Nos: 209-38637);
b) introducing the composition into the host cell
c) optionally selecting for the host cell comprising the modification or the donor nucleic acid sequence into the host cell genome; and
d) optionally culturing the host cells under conditions sufficient for growth.

32. The method of paragraph 31, wherein the nucleic acid sequence encoding the engineered TnpB protein is
a. operably fused to one or more nucleic acid encoding an endonuclease;
b. operably fused to one or more nucleic acid encoding a deaminase;
c. operably fused to one or more nucleic acid encoding a reverse transcriptase; or
d. operably fused to one or more nucleic acid encoding a transcriptional modulating polypeptide;
e. operably fused to any combination of a, b, c and/or d.

33. The method of paragraph 32 wherein the modification of the target region of the host cell genome comprises binding activity, cleavage activity, nickase activity, transcriptional activation activity, transcriptional inhibitory activity, or transcriptional epigenetic activity.

34. The method of paragraph 31 further comprising quantifying editing of the target region.

35. The method of paragraphs 31 wherein the method provides editing efficiency of greater than 10%, 20%, 30%, 40%, 50%, 60%, 70%, 80%, 90%, 95%, 99% relative to SpCas9.

36. The method of paragraph 31 further comprising introducing into the host cell a second donor nucleic acid sequence paired with a second recombinant reRNA to modify the second target region of the host cell genome.

37. The method of paragraph 31 further comprising introducing into the host cell at least two desired modification sequences for multiplexing.

38. The method of paragraph 31 wherein the method comprises insertion or stable integration of the one or more desired modification sequence into the host cell genome.

39. The method of paragraph 31 wherein the host cell genome comprises a chromosome or chromosome and plasmid.

40. The method of paragraph 31 wherein the target region is modified by an insertion, deletion or alteration of one or more base pairs at the target region in the host cell genome.

41. The method of paragraph 31 wherein the one or more desired modification sequence is selected from one or more sequences associated with one or more monogenic disorders or diseases.

42. The method of paragraph 31 wherein the host cell is a primary human cell.

43. The method of paragraph 31 wherein the step of introducing into the host cell comprises a delivery vector operably linked to the genome editing system.

44. The method of paragraph 43 wherein the delivery vector is selected from viral vector is selected from a retroviral vector, a lentiviral vector, an adenoviral, an adeno-associated viral vector, vaccinia viral vector, poxviral vector, and herpes simplex viral vector.

45. The method of paragraph 43 wherein the delivery vector comprises a non-viral vectors selected from cationic liposomes, lipid nanoparticles (LNPs), cationic polymers, vesicles, and gold nanoparticles.

46. The method of paragraph 31 wherein the editing method results in enhanced editing efficiency and/or low cytotoxicity.

47. The method of paragraph 31 wherein the method comprises a high-throughput editing of the target region of the host cell genome.

48. The method of paragraph 31 wherein the method comprises plating and/or culturing or subculturing in liquid.

49. The method of paragraph 31 wherein the recombinant reRNA comprises one or more chemical modifications selected from 2′-O-Me, 2′-F, and 2′F-ANA at 2′OH; 2′F-4′-Cα-OMe and 2′,4′-di-Cα-OMe at 2′ and 4′ carbons; phosphodiester modifications comprising sulfide-based Phosphorothioate (PS) or acetate-based phosphonoacetate alterations; combinations of the ribose and phosphodiester modifications; locked nucleic acid (LNA), bridged nucleic acids (BNA), S-constrained ethyl (cEt), and unlocked nucleic acid (UNA); modifications to produce a phosphodiester bond between the 2′ and 5′ carbons (2′,5′-RNA) of adjacent RNAs; and a butane 4-carbon chain link between adjacent RNAs.

50. A construct comprising:
a) an TnpB endonuclease; b) a deaminase; c) a reverse transcriptase; d) a transcriptional modulating polypeptide; or e) any combination of a, b, c and/or d.

51. The construct of paragraph 50 further comprising:
a nucleic acid sequence encoding a recombinant reRNA comprising a truncated reRNA of of Table D (SEQ ID NOs: 38838-77066), Table E (SEQ ID NOs: 77067-115495), or Table F (SEQ ID Nos: 115496-153924) and a guide RNA;
wherein the TnpB endonuclease and the second nucleic acid sequence form a RNA-protein complex; and
wherein the genome editing system optionally further comprises a donor nucleic acid sequence capable of modifying a target sequence; and
wherein the TnpB sequence is optionally a corresponding polypeptide from Table C(SEQ ID Nos: 209-38637).

52. The construct of paragraph 50 further comprising one or more additional nucleic acid sequence encoding one or more donor nucleic acid sequence paired with one or more nucleic acid sequence encoding a recombinant reRNA.

53. The construct of paragraph 52 wherein the donor nucleic acid sequence provides a modification to the target sequence of the host cell genome.

54. The construct of paragraph 53 wherein the target sequence is modified by an insertion, deletion or alteration of one or more base pairs at the target region in the host cell genome.

55. The construct of paragraph 53 wherein the modification is selected from one or more sequences associated with one or more monogenic disorders or diseases.

56. A recombinant host cell comprising the nucleic acid construct of any one of paragraphs 50-55.

### BRIEF DESCRIPTION OF THE DRAWINGS

FIG. 1A provides a schematic of a canonical genomic TnpA/TnpB transposable element comprising from the 5′ end to the 3′ end: a (i) left end (LE) region demarking the left-most boundary of the transposable element; (ii) a TnpA gene; (iii) a TnpB gene; and (iv) a right end (RE) region demarking the right-most boundary of the transposable element. The TnpA gene product is a transposase. The TnpB gene product is an RNA-guided nuclease which complexes with the TnpB ncRNA (or reRNA), whose transcript overlaps with 3′ end of the TnpB gene and extends into 3′ flanking (F) genomic region. The ncRNA includes a scaffold region and a guide region. The scaffold region (˜100-200 bp) complexes with the TnpB protein. The guide region is formed from continued transcription beyond the RE boundary terminating at a point that is about 16-23 nucleotides past the RE 3′-end boundary. The guide sequence facilitates the localization of the TnpB to a target sequence (also comprising a TAM site) that is complementary to the guide sequence. Once complexed at target site, the TnpB protein catalyzes a nuclease cut of both strands of the target DNA.

FIG. 1B provides a schematic of a TnpB complexed with an engineered TnpB ncRNA comprising an engineered guide that comprises a sequence that is complementary to a target DNA sequence.

FIG. 1C provides a schematic of a localized TnpB RNP complex having a TnpB ncRNA annealed at its guide RNA to a target DNA. The black arrows depict the general position of strand cutting by the TnpB nuclease.

FIG. 2 provides a schematic of an embodiment of an LNP composition comprising a ncRNA component (or a nucleic acid encoding same) and one or more coding RNAs (e.g., circular or linear RNA) which encode the TnpB nuclease and optionally one or more accessory proteins (e.g., a deaminase, reverse transcriptase, recombinase, nuclease, or integrase). Although not depicted, the LNP composition may also include a template DNA molecule (single or double stranded HDR donor molecule). As shown, the LNP composition comprising the TnpB editing system may be delivered to a cell. Once the components are delivered and/or expressed accordingly, they undergo translocation to the nucleus where they act on the target DNA to under editing (e.g., a precise nuclease cut of a target sequence). The delivery may be in vivo delivery in certain embodiments, as well as in vitro or ex vivo.

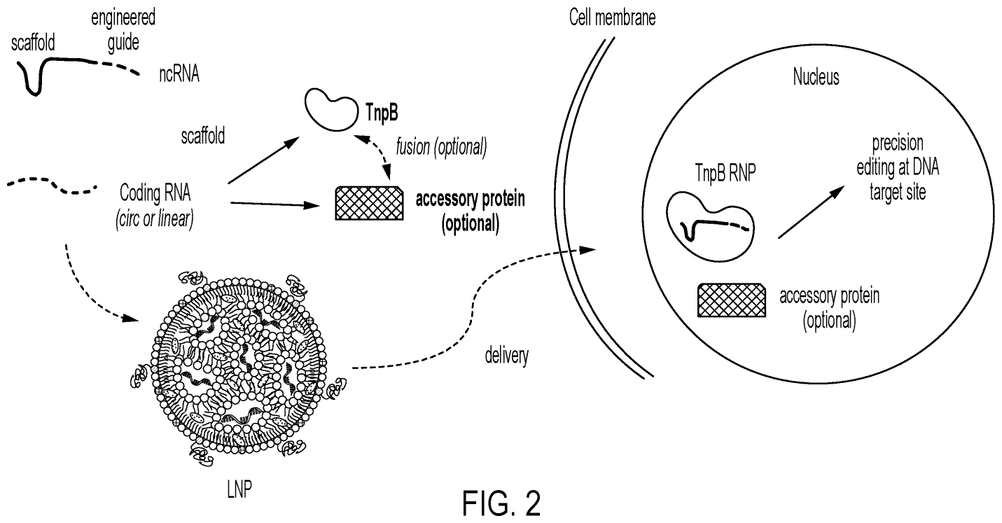

FIG. 3 illustrates various embodiments of modified TnpB proteins that are fused to one or more other accessory functions (e.g., those exemplary functions listed in Table C, including deaminases, reverse transcriptases, recombinases, nucleases, or integrases).

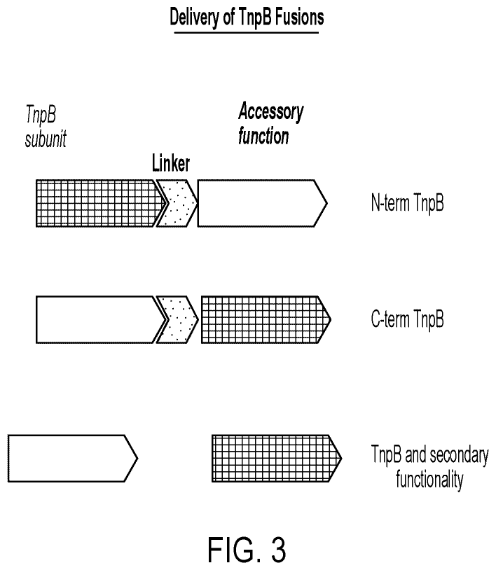

FIG. 4 illustrates the modification of a protein disclosed herein (e.g., a TnpB protein) with one or more nuclear localization sequences (NLS) to facilitate nuclear localization of the protein was in the cell (e.g., after it is translated in the cell from a delivered coding mRNA).

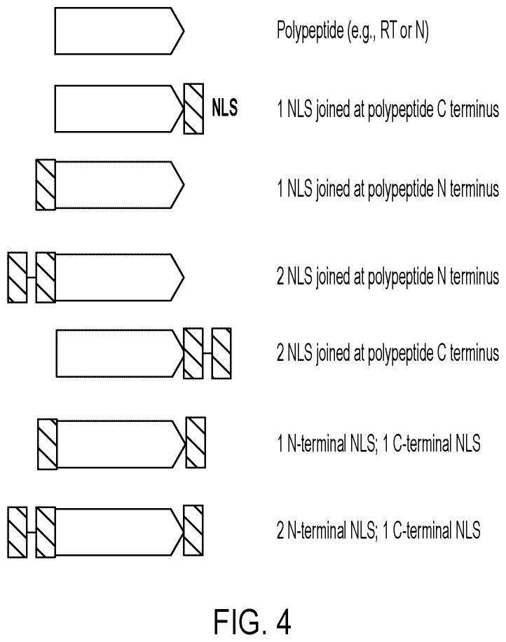

FIG. 5 demonstrates TnpB (SEQ ID NO: 1) endonuclease edits human EMX1 locus (hEMX1) in HEK293T cells.

FIG. 6 shows the most common indels created at the human EMX1 locus as detected by NGS. non-targeted strand (NTS), targeted strand (TS), transposon-associated motif (TAM) in underlined, spacer in box.

FIG. 7 demonstrates TnpB endonuclease edits Mus musculus EMX1 locus (mEMX1) in liver in vivo when delivered with an LNP (Table (III) Compound C59).

FIG. 8 shows two of the most common indels created at the mouse EMX1 locus as detected by NGS. non-targeted strand (NTS), targeted strand (TS), transposon-associated motif (TAM) in underlined, spacer in box.

### DETAILED DESCRIPTION

This specification describes novel TnpB-based genome editing systems (e.g., genome editing systems) for use in various applications, including precision gene editing in cells, tissues, organs, or organisms. In various embodiments, the TnpB-based genome editing systems comprise (a) a TnpB polypetide and (b) a TnpB guide RNA (or reRNA) which is capable of associating with the TnpB polypeptide to form a complex such that the complex localizes to a target nucleic acid sequence (e.g., a genomic or plasmid target sequence) and binds thereto. In various embodiments, the reRNA may comprise one or more targeting sequences that have complementarity with a target nucleic acid sequence (e.g., a specific genomic locus). Without being bound by theory, the inventors have surprising discovered a large set of novel predicted reRNAs associated with known TnpB polypeptides. The novel reRNA, and engineered or modified versions thereof, may be combined with the herein described TnpB polypeptides, and optionally one or more additional accessory functional proteins (e.g., deaminase, nuclease, reverse transcriptase, invertase, or polymerase) to form various formats envisioned for the herein disclosed TnpB-based genome editing systems (e.g., genome editing systems) for use in various applications, including precision gene editing in cells, tissues, organs, or organisms. The present disclosure further relates to nucleic acid molecules encoding the novel TnpB-based genome editing systems (e.g., genome editing systems), isolated protein components of the TnpB-based genome editing systems (e.g., genome editing systems) described herein, guide RNAs suitable for programming the herein disclosed TnpB proteins to target and bind to a specific target nucleotide sequence, including the novel reRNA molecules identified in Tables D (SEQ ID Nos.: 8258-16306), E (SEQ ID Nos: 16307-24355), and F (SEQ ID Nos: 24356-32404), delivery systems to delivery the TnpB-based genome editings systems (in the form of RNA, DNA, protein, or complexes thereof) to cells, tissues, organs, or organisms, and methods of using the TnpB-based genome editing systems in their various envisioned formats to conduct genome editing, including introducing nucleic acid insertions, deletions, substitutions, inversion into target nucleic acid molecules (e.g., a genome).

a. Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

### A

The articles “a” and “an” are used herein to refer to one or to more than one (i.e., to at least one) of the grammatical object of the article. By way of example, “an element” means one element or more than one element.

### About

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

### Antibody

As used herein, the term “antibody” is referred to in the broadest sense and specifically covers various embodiments including, but not limited to monoclonal antibodies, polyclonal antibodies, multispecific antibodies (e.g., bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.). Non-limiting examples of antibodies or fragments thereof include VH and VL domains, scFvs, Fab, Fab′, F(ab′)₂, Fv fragment, diabodies, linear antibodies, single chain antibody molecules, multispecific antibodies, bispecific antibodies, intrabodies, monoclonal antibodies, polyclonal antibodies, humanized antibodies, codon-optimized antibodies, tandem scFv antibodies, bispecific T-cell engagers, mAb2 antibodies, chimeric antigen receptors (CAR), tetravalent bispecific antibodies, biosynthetic antibodies, native antibodies, miniaturized antibodies, unibodies, maxibodies, antibodies to senescent cells, antibodies to conformers, antibodies to disease specific epitopes, or antibodies to innate defense molecules.

### Biologically Active

As used herein, the term “biologically active” refers to a characteristic of an agent (e.g., DNA, RNA, or protein) that has activity in a biological system (including in vitro and in vivo biological system), and particularly in a living organism, such as in a mammal, including human and non-human mammals. For instance, an agent when administered to an organism has a biological effect on that organism, is considered to be biologically active.

### Bulge

As used herein, the term “bulge” refers to a small region of unpaired base(s) that interrupts a “stem” of base-paired nucleotides. The bulge may comprise one or two single-stranded or unbase-paired nucleotides joined at both ends by base-paired nucleotides of the stem. The bulge can be symmetrical (viz., the two unbase-paired single-stranded regions have the same number of nucleotides), or asymmetrical (viz., the unbase-paired single stranded region(s) have different or unequal numbers of nucleotides), or there is only one unbase-paired nucleotide on one strand. A bulge can be described as A/B (such as a “2/2 bulge,” or a “I/O bulge”) wherein A represents the number of unpaired nucleotides on the upstream strand of the stem, and B represents the number of unpaired nucleotides on the downstream strand of the stem. An upstream strand of a bulge is more 5′ to a downstream strand of the bulge in the primary nucleotide sequence.

### CDNA

As used hereing, the term “cDNA” refers to a strand of DNA copied from an RNA template, e.g., by a reverse transcriptase.

### Complementary

As used herein, the terms “complementary” or “substantially complementary” are meant to refer to a nucleic acid (e.g., RNA, DNA) that comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

### DNA

The term “DNA” is a well-known term of art that refers to deoxyribonucleic acid.

### DNA-Guided Nuclease

As used herein, an “DNA-guided nuclease” is a type of “programmable nuclease,” and a specific type of “nucleic acid-guided nuclease.” An example of a DNA-guided nuclease is reported in Varshney et al., DNA-guided genome editing using structure-guided endonucleases, Genome Biology, 2016, 17 (1), 187, which may be used in the context of the present disclosure and is incorporated herein by reference. As used herein, the term “DNA-guided nuclease” or “DNA-guided endonuclease” refers to a nuclease that associates covalently or non-covalently with a guide RNA thereby forming a complex between the guide RNA and the DNA-guided nuclease. The guide RNA comprises a spacer sequence which comprises a nucleotide sequence having complementarity with a strand of a target DNA sequence. Thus, the DNA-guided nuclease is indirectly guided or programmed to localize to a specific site in a DNA molecule through its association with the guide RNA, which directly binds or anneals to a strand of the target DNA through its complementarity region via Watson-Crick base-pairing.

### DNA Regulatory Sequences

As used herein, the terms “DNA regulatory sequences,” “control elements,” and “regulatory elements,” can be used interchangeably herein to refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence and/or regulate translation of a mRNA into an encoded polypeptide.

### Donor Nucleic Acid

By a “donor nucleic acid” or “donor polynucleotide” or “donor DNA” or “HDR donor DNA” it is meant a single-stranded DNA to be inserted at a site cleaved by a programmable nuclease (e.g., a CRISPR/Cas effector protein; a TALEN; a ZFN; a meganuclease)(e.g., after dsDNA cleavage, after nicking a target DNA, after dual nicking a target DNA, and the like). The donor polynucleotide can contain sufficient homology to a genomic sequence at the target site, e.g. 70%, 80%, 85%, 90%, 95%, or 100% homology with the nucleotide sequences flanking the target site, e.g., within about 200 bases or less of the target site, e.g., within about 190 bases or less of the target site, e.g., within about 180 bases or less of the target site, e.g., within about 170 bases or less of the target site, e.g., within about 160 bases or less of the target site, e.g., within about 150 bases or less of the target site, e.g., within about 140 bases or less of the target site, e.g., within about 130 bases or less of the target site, e.g., within about 120 bases or less of the target site, e.g., within about 110 bases or less of the target site, e.g., within about 100 bases or less of the target site, e.g., within about 90 bases or less of the target site, e.g., within about 80 bases or less of the target site, e.g., within about 70 bases or less of the target site, e.g., within about 60 bases or less of the target site, e.g., 50 bases or less of the target site, e.g., within about 30 bases, within about 15 bases, within about 10 bases, within about 5 bases, or immediately flanking the target site, to support homology-directed repair between it and the genomic sequence to which it bears homology.

### Effective Amount

An “effective amount” as used herein, means an amount which provides a therapeutic or prophylactic benefit under the conditions of administration.

### Encapsulation Efficiency

As used herein, “encapsulation efficiency” refers to the amount of a therapeutic and/or prophylactic that becomes part of a nanoparticle composition, relative to theinitial total amount of therapeutic and/or prophylactic used in the preparation of a nanoparticle composition. For example, if 97 mg of a polynucleotide are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Throughout the disclosure, chemical substituents described in Markush structures are represented by variables. Where a variable is given multiple definitions as applied to different Markush formulas in different sections of the disclosure, it is to be understood that each definition should only apply to the applicable formula in the appropriate section of the disclosure.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

As used herein, the following abbreviations and initialisms have the indicated meanings:

| | |
|---|---|
| MC3 | 4-(dimethylamino)-butanoic acid, (10Z,13Z)-1-(9Z,12Z)-9,12- |
| | octadecadien-1-yl-10,13-nonadecadien-1-yl ester |
| DSPC | 1,2-distearoyl-sn-glycero-3-phosphocholine |
| DMG | 1,2-Dimyristoyl-rac-glycero-3-methanol |
| DOMG- | R-3-[(ω-methoxy-poly(ethyleneglycol))carbamoyl)]-1,2- |
| PEG | dimyristyloxypropyl-3-amine |
| DLPE | 1,2-Dilauroyl-sn-Glycero-3-Phosphoethanolamine |
| DMPE | 1,2-Dimyristoyl-sn-Glycero-3-Phosphoethanolamine |
| DPPC | 1,2-dipalmitoyl-sn-glycero-3-phosphocholine |
| DSPE | 1,2-distearoyl-sn-glycero-3-phosphoethanolamine |
| DDAB | Didodecyldimethylammonium bromide |
| EPC | 1,2-dioleoyl-sn-glycero-3-ethylphosphocholine |
| 14PA | 1,2-dimyristoyl-sn-glycero-3-phosphate |
| 18BMP | bis(monooleoylglycero)phosphate |
| DODAP | 1,2-dioleoyl-3-dimethylammonium-propane |
| DOTAP | 1,2-dioleoyl-3-trimethylammonium-propane |
| C12- | 1,1′-((2-(4-(2-((2-(bis(2-hydroxydodecyl)amino)ethyl)(2- |
| 200 | hydroxydodecyl)amino)ethyl)piperazin-1- |
| | yl)ethyl)azanediyl)bis(dodecan-2-ol) |

### Encoding

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

### Exosome

As used herein, the term “exosomes” refer to small membrane bound vesicles with an endocytic origin. Without wishing to be bound by theory, exosomes are generally released into an extracellular environment from host/progenitor cells post fusion of multivesicular bodies the cellular plasma membrane. As such, exosomes can include components of the progenitor membrane in addition to designed components (e.g. engineered TnpB editing system). Exosome membranes are generally lamellar, composed of a bilayer of lipids, with an aqueous inter-nanoparticle space.

### Expression Vector

As used herein, the term “expression vector” or “expression construct” refers to a vector that includes one or more expression control sequences, and an “expression control sequence” is a DNA sequence that controls and regulates the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available, such as from Novagen (Madison, WI), Clontech (Palo Alto, CA), Stratagene (La Jolla, CA), and Invitrogen/Life Technologies (Carlsbad, CA). The present invention comprehends recombinant vectors that may include viral vectors, bacterial vectors, protozoan vectors, DNA vectors, or recombinants thereof.

### Heterologous Nucleic Acid

As used herein, the term “heterologous nucleic acid” refers to a genotypically distinct entity from that of the rest of the entity to which it is compared or into which it is introduced or incorporated. For example, a polynucleotide introduced by genetic engineering techniques into a different cell type is a heterologous polynucleotide (e.g., DNA or RNA) and, if expressed, can encode a heterologous polypeptide. Similarly, a cellular sequence (e.g., a gene or portion thereof) that is incorporated into a viral vector is a heterologous nucleotide sequence with respect to the vector. In certain embodiments, the heterologous sequence is a mammalian sequence (e.g., a human sequence), or a reverse complement thereof. Heterologous nucleic acid sequences can be introduced into reRNA (i.e., TnpB guide RNAs) and can include without limitation guide RNA sequences, targeting sequences, donor templates, protein-encoding genes, or non-coding functional RNA elements (e.g., stem-loops, hairpins, and bulges).

### Homologous

As used herein, the term “homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

### Identical

As used herein, the term “identical” refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using a comparison algorithm or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence.

### Isolated

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

### Isolated Nucleic Acid

An “isolated nucleic acid” refers to a nucleic acid segment or fragment, which has been separated from sequences which flank it in a naturally occurring state, i.e., a DNA fragment, which has been removed from the sequences which are normally adjacent to the fragment, i.e., the sequences adjacent to the fragment in a genome in which it naturally occurs. The term also applies to nucleic acids which have been substantially purified from other components, which naturally accompany the nucleic acid, i.e., RNA or DNA or proteins, which naturally accompany it in the cell. The term therefore includes, for example, a recombinant DNA or RNA, which is incorporated into a vector, into an autonomously replicating plasmid or virus, or into the genomic DNA or RNA of a prokaryote or eukaryote, or which exists as a separate molecule (i.e., as a cDNA or a genomic or cDNA fragment produced by PCR or restriction enzyme digestion) independent of other sequences. It also includes a recombinant DNA or RNA, which is part of a hybrid gene encoding additional polypeptide sequence.

### Lipid Nanoparticle or LNP

As used herein, the term “lipid nanoparticle” or LNP refers to a type of lipid particle delivery system formed of small solid or semi-solid particles possessing an exterior lipid layer with a hydrophilic exterior surface that is exposed to the non-LNP environment, an interior space which may aqueous (vesicle like) or non-aqueous (micelle like), and at least one hydrophobic inter-membrane space. LNP membranes may be lamellar or non-lamellar and may be comprised of 1, 2, 3, 4, 5 or more layers. In some embodiments, LNPs may comprise a nucleic acid (e.g. engineered TnpB editing system) into their interior space, into the inter membrane space, onto their exterior surface, or any combination thereof. In some embodiments, an LNP of the present disclosure comprises an ionizable lipid, a structural lipid, a PEGylated lipid (aka PEG lipid), and a phospholipid. In alternative embodiments, an LNP comprises an ionizable lipid, a structural lipid, a PEGylated lipid (aka PEG lipid), and a zwitterionic amino acid lipid.

### Linker

As used herein, the term “linker” refers to a molecule linking or joining two other molecules or moieties. The linker can be an amino acid sequence in the case of a linker joining two fusion proteins. For example, a TnpB protein can be fused to an accessory protein (e.g., a deaminase, nuclease, ligase, reverse transcriptase, recombinase, etc.) by an amino acid linker sequence.

The linker can also be a nucleotide sequence in the case of joining two nucleotide sequences together. For example, in the instant case, a reRNA at its 5′ and/or 3′ ends may be linked by a nucleotide sequence linker to one or more other functional nucleic acid molecules, such as guide RNAs or HDR donor molecules.

In other embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

### Liposomes

As used herein, the term “liposomes” refer to small vesicles that contain at least one lipid bilayer membrane surrounding an aqueous inner-nanoparticle space that is generally not derived from a progenitor/host cell. Further discuss of liposomes can be found, for example, in Tenchov et al., “Lipid Nanoparticles—From Liposomes to mRNA Vaccine Delivery, a Landscape of Diversity and Advancement,” ACS Nano, 2021, 15, pp. 16982-17015 (the contents of which are incorporated by reference).

### Micelle

As used herein, the term “micelles” refer to small particles which do not have an aqueous intra-particle space.

### Modulating

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

### Nanoparticle

As used herein, the term “nanoparticle” refers to any particle ranging in size from 10-1,000 nm.

### Nuclear Localization Sequence (NLS)

As used herein, the term “nuclear localization sequence” or “NLS” refers to an amino acid sequence that promotes import of a protein (e.g., a RNA-guided nuclease) into the cell nucleus, for example, by nuclear transport. Nuclear localization sequences are known in the art. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences.

### Nucleic Acid

As used herein, the term “nucleic acid” or “nucleic acid molecule” or “nucleic acid sequence” or “polynucleotide” generally refer to deoxyribonucleic or ribonucleic oligonucleotides in either single- or double-stranded form. The term may (or may not) encompass oligonucleotides containing known analogues of natural nucleotides. The term also may (or may not) encompass nucleic acid-like structures with synthetic backbones, see, e.g., Eckstein, 1991; Baserga et ah, 1992; Milligan, 1993; WO 97/03211; WO 96/39154; Mata, 1997; Strauss-Soukup, 1997; and Samstag, 1996. The term encompasses both ribonucleic acid (RNA) and DNA, including cDNA, genomic DNA, synthetic, synthesized (e.g., chemically synthesized) DNA, and/or DNA (or RNA) containing nucleic acid analogs. The nucleotides Adenine (A), Thymine (T), Guanine (G) and Cytosine (C) also may (or may not) encompass nucleotide modifications, e.g., methylated and/or hydroxylated nucleotides, e.g., Cytosine (C) encompasses 5-methylcytosine and 5-hydroxymethylcytosine.

### Nucleic Acid Loop

As used herein, the term “loop” in the polynucleotide refers to a single stranded stretch of one or more nucleotides, such as 2, 3, 4, 5, 6, 7, 8, 9, or 10 nucleotides, wherein the most 5′ nucleotide and the most 3′ nucleotide of the loop are each linked to a base-paired nucleotide in a stem.

### Nucleic Acid Stem

As used herein, the term “stem” refers to two or more base pairs, such as 3, 4, 5, 6, 7, 8, 9, 10, 11, 12, 13, 14, 15, 16, 17, 18, 19, 20 or more base pairs, formed by inverted repeat sequences connected at a “tip,” where the more 5′ or “upstream” strand of the stem bends to allows the more 3′ or “downstream” strand to base-pair with the upstream strand. The number of base pairs in a stem is the “length” of the stem. The tip of the stem is typically at least 3 nucleotides, but can be 4, 5, 6, 7, 8, 9, 10, 11, 12, 13, 14, 15 or more nucleotides. Larger tips with more than 5 nucleotides are also referred to as a “loop.” An otherwise continuous stem may be interrupted by one or more bulges as defined herein. The number of unpaired nucleotides in the bulge(s) are not included in the length of the stem. The position of a bulge closest to the tip can be described by the number of base pairs between the bulge and the tip (e.g., the bulge is 4 bps from the tip). The position of the other bulges (if any) further away from the tip can be described by the number of base pairs in the stem between the bulge in question and the tip, excluding any unpaired bases of other bulges in between.

### Operably Linked

As used herein, the term “operably linked” or “under transcriptional control,” when used in conjunction with the description of a promoter, refers to the correct location and orientation in relation to a polynucleotide (e.g., a coding sequence) to control the initiation of transcription by RNA polymerase and expression of the coding sequence, such as one for the msr gene, msd gene, and/or the ret gene.

### PEG Lipid

As used herein, a “PEG lipid” or “PEGylated lipid” refers to a lipid comprising a polyethylene glycol component.

### Programmable Nuclease

As used herein, the term “programmable nuclease” is meant to refer to a polypeptide that has the property of selective localization to a specific desired nucleotide sequence target in a nucleic acid molecule (e.g., to a specific gene target) due to one or more targeting functions. Such targeting functions can include one or more DNA-binding domains, such as zinc finger domains characteristic of many different types of DNA binding proteins or TALE domains characteristic of TALEN proteins. Such targeting function may also include the ability to associate and/or form a complex with a guide RNA, which then localizes to a specific site on the DNA which bears a sequence that is complementary to a portion of the guide RNA (i.e., the spacer of the guide RNA). In some embodiments, the programmable nuclease may be a single protein which comprises both a domain that binds directly (e.g., a ZF protein) or indirectly (e.g., an RNA-guided protein) to a target DNA site, as well as a nuclease domain. In other embodiments, the programmable nuclease may be a composite of two or more separate proteins or domains (from different proteins) which together provide the necessary functions of selective DNA binding and nuclease activity. For example, the programmable nuclease may comprise a (a) nuclease-inactive RNA-guided nuclease (which still is capable of binding a guide RNA, localizing to a target DNA, and binding to the target DNA, but not capable of cutting or nicking the strands) fused to a (b) nuclease protein or domain, such as a FokI nuclease.

### Polypeptide

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

### Recombinant Nucleic Acid

A “recombinant nucleic acid” or “recombinant nucleotide” refers to a molecule that is constructed by joining nucleic acid molecules, which optionally may self-replicate in a live cell.

### RNA

The term “RNA” is a well-known term of art that refers to ribonucleic acid.

### RNA-Guided Nuclease

As used herein, an “RNA-guided nuclease” is a type of “programmable nuclease,” and a specific type of “nucleic acid-guided nuclease.” As used herein, the term “RNA-guided nuclease” or “RNA-guided endonuclease” refers to a nuclease that associates covalently or non-covalently with a guide RNA thereby forming a complex between the guide RNA and the RNA-guided nuclease. The guide RNA comprises a spacer sequence which comprises a nucleotide sequence having complementarity with a strand of a target DNA sequence. Thus, the RNA-guided nuclease is indirectly guided or programmed to localize to a specific site in a DNA molecule through its association with the guide RNA, which directly binds or anneals to a strand of the target DNA through its complementarity region via Watson-Crick base-pairing.

### Sequence Identity

As used herein, the term “sequence identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). For example, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna. CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12 (1), 387 (1984)), BLASTP, BLASTN, and FASTA Altschul, S. F. et al., J. Molec. Biol., 215, 403 (1990).

### Subject

As used herein, the term “subject” refers to an individual organism, for example, an individual mammal or plant. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. The terms “individual,” “subject,” “host,” and “patient,” used interchangeably herein.

### Synthetic or Artificial Nucleic Acid

A “synthetic or artificial nucleic acid” refers nucleic acids that are non-naturally occurring sequences. Such sequences do not originate from, or are not known to be present in any living organism (e.g., based on sequence search in existing sequence databases).

Recombinant nucleic acids and synthetic nucleic acids also include those molecules that result from the replication of either of the foregoing.

Engineered nucleic acid constructs of the present disclosure, such as the engineered TnpB systems described herein, may be encoded by a single molecule (e.g., encoded by or present on the same plasmid or other suitable vector) or by multiple different molecules (e.g., multiple independently-replicating vectors).

### Target Site

As used herein, a “target site” as used herein is a polynucleotide (e.g., DNA such as genomic DNA) that includes a site or specific locus (“target site” or “target sequence”) targeted by a TnpB editing system disclosed herein. In the context of TnpB-based genome modification systems disclosed herein that comprise an RNA-guided nuclease, a target sequence is the sequence to which the guide sequence of a guide nucleic acid (e.g., guide RNA or reRNA) will hybridize. For example, the target site (or target sequence) 5′-GTCAATGGACC-3′ within a target nucleic acid is targeted by (or is bound by, or hybridizes with, or is complementary to) the sequence 5′-GGTCCATTGAC-3′. Suitable hybridization conditions include physiological conditions normally present in a cell. For a double stranded target nucleic acid, the strand of the target nucleic acid that is complementary to and hybridizes with the guide RNA is referred to as the “complementary strand” or “target strand”; while the strand of the target nucleic acid that is complementary to the “target strand” (and is therefore not complementary to the guide RNA) is referred to as the “non-target strand” or “non-complementary strand.” For purposes of this application, the reRNA described herein may be referred to as guide RNA that are compatible with TnpBs.

### Therapeutic

The term “therapeutic” as used herein means a treatment and/or prophylaxis. A therapeutic effect is obtained by suppression, diminution, remission, or eradication of at least one sign or symptom of a disease or disorder state.

### Therapeutically Effective Amount

The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

### Treat

To “treat” a disease as the term is used herein, means to reduce the frequency or severity of at least one sign or symptom of a disease or disorder experienced by a subject.

### Upstream and Downstream

As used herein, the terms “upstream” and “downstream” are terms of relativity that define the linear position of at least two elements located in a nucleic acid molecule (whether single or double-stranded) that is orientated in a 5′- to -3′ direction. A first element is said to be upstream of a second element in a nucleic acid molecule where the first element is positioned somewhere that is 5′ to the second element. Conversely, a first element is downstream of a second element in a nucleic acid molecule where the first element is positioned somewhere that is 3′ to the second element.

### Variant

As used herein the term “variant” should be taken to mean the exhibition of qualities that have a pattern that deviates from what occurs in nature, e.g., a variant TnpB is TnpB comprising one or more changes in amino acid residues as compared to a TnpB amino acid sequence. The term “variant” encompasses homologous proteins having at least 75%, or at least 80%, or at least 85%, or at least 90%, or at least 95%, or at least 99% percent identity with a reference sequence and having the same or substantially the same functional activity or activities as the reference sequence. The term also encompasses mutants, truncations, or domains of a reference sequence, and which display the same or substantially the same functional activity or activities as the reference sequence.

### Vector

As used herein, the term “vector” permits or facilitates the transfer of a polynucleotide from one environment to another. It is a replicon such as a plasmid, phage, or cosmid into which another DNA segment may be inserted so as to bring about the replication of the inserted segment (e.g., the subject engineered TnpB systems). Generally, a vector is capable of replication when associated with the proper control elements. The term “vector” may include cloning and expression vectors, as well as viral vectors and integrating vectors.

### Chemical Terms

“Alkyl” refers to a straight or branched hydrocarbon chain radical consisting solely of carbon and hydrogen atoms, which is saturated or unsaturated (i.e., contains one or more double and/or triple bonds), having from one to thirty or more carbon atoms (e.g., C₁-C₂₄ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond, e.g., methyl, ethyl, n propyl, 1-methylethyl (iso propyl), n butyl, n pentyl, 1,1 dimethylethyl (t butyl), 3 methylhexyl, 2 methylhexyl, ethenyl, propyl enyl, but-1-enyl, pent-1-enyl, penta-1,4-dienyl, ethynyl, propynyl, butynyl, pentynyl, hexynyl, and the like. Alkyl groups that include one or more units of unsaturation (one or more double and/or triple bond) can be C₂-C₂₄, C₂-C₁₂, C₂-C₈ or C₂-C₆ groups, for example. Unless specifically stated otherwise, an alkyl group is optionally substituted. The term “alkyl,” by itself or as part of another substituent means, unless otherwise stated, a straight or branched chain hydrocarbon having the number of carbon atoms designated (i.e., C₁₋₆ means one to six carbon atoms) and includes straight, branched chain, or cyclic substituent groups.

“Alkylene” or “alkylene chain” refers to a straight or branched divalent hydrocarbon chain consisting solely of carbon and hydrogen, which is saturated or unsaturated (i.e., contains one or more double (alkenylene) and/or triple bonds (alkynylene)), and having, for example, from one to thirty or more carbon atoms (e.g., C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene), e.g., methylene, ethylene, propylene, n-butylene, ethenylene, propenylene, n-butenylene, propynylene, n-butynylene, and the like. Alkylene groups that include one or more units of unsaturation (one or more double and/or triple bond) can be C₂-C₂₄, C₂-C₁₂, C₂-C₈ or C₂-C₆ groups, for example. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Cycloalkyl” or “carbocyclic ring” refers to a stable non aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbomyl, decalinyl, 7,7 dimethyl bicyclo[2.2.1]heptanyl, and the like. Unless specifically stated otherwise, a cycloalkyl group is optionally substituted.

“Cycloalkylene” is a divalent cycloalkyl group. Unless otherwise stated specifically in the specification, a cycloalkylene group may be optionally substituted.

As used herein, the term “heteroalkyl” by itself or in combination with another term means, unless otherwise stated, a stable straight or branched chain alkyl group consisting of the stated number of carbon atoms and one or two or more heteroatoms typically selected from the group consisting of O, N, Si, P, and S, and wherein the nitrogen and sulfur atoms may be optionally oxidized and the nitrogen heteroatom may be a primary, secondary, tertiary or quaternary nitrogen. The heteroatom(s) may be placed at any position of the heteroalkyl group, including between the rest of the heteroalkyl group and the fragment to which it is attached, as well as attached to the most distal carbon atom in the heteroalkyl group. Examples of heteroalkyl groups include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heterocyclyl” or “heterocyclic ring” refers to a stable 3- to 18-membered non-aromatic ring radical which consists of two to twelve carbon atoms and from one to six heteroatoms typically selected from the group consisting of N, O, Si, P, and S. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless specifically stated otherwise, a heterocyclyl group may be optionally substituted.

As used herein, the term “aromatic” refers to a carbocycle or heterocycle with one or more polyunsaturated rings and having aromatic character, i.e. having (4n+2) delocalized p (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “heteroaryl” or “heteroaromatic” refers to aryl groups which contain at least one heteroatom typically selected from N, O, Si, P, and S; wherein the nitrogen and sulfur atoms may be optionally oxidized, and the nitrogen atom(s) may be optionally teriatry or quaternized. Heteroaryl groups may be substituted or unsubstituted. A heteroaryl group may be attached to the remainder of the molecule through a heteroatom. A polycyclic heteroaryl may include one or more rings that are partially saturated. Examples include tetrahydroquinoline, 2,3-dihydrobenzofuryl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide. Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl. Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl. The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, quaternary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the terms “alkoxy,” “alkylamino” and “alkylthio” are used in their conventional sense, and refer to alkyl groups linked to molecules via an oxygen atom, an amino group, a sulfur atom, respectively.

For example, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As described herein, compounds of the present disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R°; —(CH₂)₀₋₄OR°; —O(CH₂)₀₋₄R°, —O—(CH₂)₀₋₄C(O)OR°; —(CH₂)₀₋₄CH(OR°)₂; —(CH₂)₀₋₄SR°; —(CH₂)₀₋₄Ph, which may be substituted with R°; —(CH₂)₀₋₄O(CH₂)₀₋₁Ph which may be substituted with R°; —CH═CHPh, which may be substituted with R°; —(CH₂)₀₋₄O(CH₂)₀₋₁-pyridyl which may be substituted with R°; —NO₂; —CN; —N₃; —(CH₂)₀₋₄N(R°)₂; —(CH₂)₀₋₄N(R°)C(O)R°; —N(R°)C(S)R°; —(CH₂)₀₋₄N(R°)C(O)NR°₂; —N(R°)C(S)NR°₂; —(CH₂)₀₋₄N(R°)C(O)OR°; —N(R°)N(R°)C(O)R°; —N(R°)N(R°)C(O)NR°₂; —N(R°)N(R°)C(O)OR°; —(CH₂)₀₋₄C(O)R°; —C(S)R°; —(CH₂)₀₋₄C(O)OR°; —(CH₂)₀₋₄C(O)SR°; —(CH₂)₀₋₄C(O)OSiR°₃; —(CH₂)₀₋₄ OC(O)R°; —OC(O)(CH₂)₀₋₄SR°, SC(S)SR°; —(CH₂)₀₋₄SC(O)R°; —(CH₂)₀₋₄C(O)NR°₂; —C(S)NR°₂; —C(S)SR°; —SC(S)SR°, —(CH₂)₀₋₄OC(O)NR°₂; —C(O)N(OR°)R°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)₂OR°; —(CH₂)₀₋₄OS(O)₂R°; —S(O)₂NR°₂; —(CH₂)₀₋₄S(O)R°; —N(R°)S(O)₂NR°₂; —N(R°)S(O)₂R°; —N(OR°)R°; —C(NH)NR°₂; —P(O)₂R°; —P(O)R°₂; —OP(O)R°₂; —OP(O)(OR°)₂; SiR°₃; —(C₁-4 straight or branched alkylene)O—N(R°)₂; or —(C₁₋₄ straight or branched) alkylene)C(O)O—N(R°)₂, wherein each R° may be substituted as defined below and is independently hydrogen, C₁₋₆ aliphatic, —CH₂Ph, —O (CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R°, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R° (or the ring formed by taking two independent occurrences of R° together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R° is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R° include ═O and ═S.

Suitable divalent substituents on a saturated carbon atom of an “optionally substituted” group include the following: ═O, ═S, ═NNR*₂, ═NNHC(O)R*, ═NNHC(O)OR*,═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁ Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each Rt is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation, for example, by rearrangement, cyclization, or elimination.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

### B. Tnpb Editing Systems

Embodiments disclosed herein provide engineered TnpB-based genome editing systems for use in various applications, including precision gene editing in cells, tissues, organs, or organisms. The TnpB-based genome editing systems comprise a TnpB polypeptide and a nucleic acid component capable of forming a complex with the TnpB polypeptide and directing the complex to a target nucleotide sequence (e.g., a genomic target sequence such as a disease-associated gene). The TnpB systems contemplated herein may also be modified with one or more additional accessory functions, such as a nuclease, recombinase, ligase, reverse transcriptase, polymerase, deaminase, etc. to provide additional genome editing functionality. In addition, the TnpB systems contemplated herein can utilize a nuclease-limited or nuclease-deficienty TnpB variant. Normal TnpB nuclease activity cuts both strands of a target DNA, however, TnpB nickases (having only the ability to cut one of the two strands but not both strands) and nuclease-inactive or “dead” TnpB (which does not cut either strand) may also be used into the TnpB systems described herein, particularly when combined with at least another genome editing functionality, such as a deaminase (for base editing functionality) or a reverse transcriptase (for prime editing functionality). Thus, disclosed herein are TnpB systems that may function as nuclease, nickases, or catalytically inactive polynucleotide binding proteins that can be coupled with other functional domains, such as deaminases, recombinase, ligases, polymerases, nucleases, or reverse transcriptases. In one embodiment, the TnpB systems and related compositions may

specifically target single-strand or double-strand DNA. In one embodiment, the TnpB system may bind and cleave double-strand DNA. In one embodiment, the TnpB system may bind to double-stranded DNA without introducing a break to either of the strands. In one embodiment, the TnpB polypeptides or nuclease/nucleic acid component complexes may open, disrupting the continuity of one of the two DNA strands, thereby introducing a nick of the double stranded DNA. In an embodiment, and without being bound by theory, the size and configuration of the TnpB systems allows exposure to the non-targeting strand, which may be in single-stranded form, to allow for for the ability to modify, edit, delet or insert polynucleotides on the non-target strand. In an embodiment, this accessibility further allows for enhanced editing outcomes on the target and/or non-target strand, e.g., increased specificity, enhanced editing efficiency.

In another aspect, embodiments disclosed herein include applications of the compositions herein, including therapeutic and diagnostic compositions and uses. Delivery of the proteins and systems disclosed is also provided, including to a variety of cells and via a variety of delivery vehicles.

### TnpB Proteins

In one aspect, embodiments disclosed herein are directed to compositions comprising a TnpB and a reRNA capable of forming a complex with the TnpB and directing site-specific binding of the TnpB to a target sequence on a target polynucleotide.

Any TnpB polypeptide may be utilized with the compositions described herein. Examples of TnpB proteins are provided as follows; however, these specific examples are not meant to be limiting. The TnpB editing systems of the present disclosure may use any suitable TnpB protein.

The TnpB editing systems disclosed herein may comprise a canonical or naturally-occurring TnpBs, or any ortholog TnpB protein, or any variant TnpB protein-including any naturally occurring variant, mutant, or otherwise engineered version of TnpB that is known or which can be made or evolved through a directed evolutionary or otherwise mutagenic process. In various embodiments, the TnpB or TnpB variant can have a nickase activity, i.e., only cleave one strand of the target DNA sequence. In other embodiments, the TnpB or TnpB variants have inactive nucleases, i.e., are “dead” TnpB proteins. Other variant TnpB proteins that may be used are those having a smaller molecular weight than the canonical TnpB (e.g., for easier delivery) or having modified amino acid sequences or substitutions.

The TnpBs contemplated herein for use in the delivery systems (e.g., LNPs) and methods described herein include TnpB proteins described in the published literature and/or which are otherwise available in the art. For example, the following references may be used in the delivery compositions and methods of the present disclosure, each of which are incorporated herein by reference in their entireties.
IS Dra 2 transposition in D einococcus radiodurans is downregulated by TnpB; Molecular Microbiology; Pasternak, Cécile; Dulermo, Rémi; Ton-Hoang, Bao; Debuchy, Robert; Siguier, Patricia; Coste, Geneviève; Chandler, Michael; Sommer, Suzanne Vol. 88 Issue 2, pp. 443-455, 2013.
An updated evolutionary classification of CRISPR-Cas systems; Nature Reviews Microbiology; Makarova, Kira S.; Wolf, Yuri I.; Alkhnbashi, Omer S.; Costa, Fabrizio; Shah, Shiraz A.; Saunders, Sita J.; Barrangou, Rodolphe; Brouns, Stan J. J.; Charpentier, Emmanuelle; Haft, Daniel H.; Horvath, Philippe; Moineau, Sylvain; Mojica, Francisco J. M.; Terns, Rebecca M.; Terns, Michael P.; White, Malcolm F.; Yakunin, Alexander F.; Garrett, Roger A.; van der Oost, John; Backofen, Rolf; Koonin, Eugene V.; Vol. 13 Issue 11, pp. 722-736, 2015.
Diversity and evolution of class 2 CRISPR-Cas systems; Nature Reviews Microbiology; Shmakov, Sergey; Smargon, Aaron; Scott, David; Cox, David; Pyzocha, Neena; Yan, Winston; Abudayyeh, Omar O.; Gootenberg, Jonathan S.; Makarova, Kira S.; Wolf, Yuri I.; Severinov, Konstantin; Zhang, Feng; Koonin, Eugene V.; Vol. 15 Issue 3, pp. 169-182, 2017.
The evolution of CRISPR/Cas9 and their cousins: hope or hype?; Biotechnology Letters; Bhushan, Kul; Chattopadhyay, Anirudha; Pratap, Dharmendra Vol. 40 Issue 3, pp. 465-477, 2018.
The Expanding Class 2 CRISPR Toolbox: Diversity, Applicability, and Targeting Drawbacks; BioDrugs; Hajizadeh Dastjerdi, Arash; Newman, Anthony; Burgio, Gaetan; Vol. 33 Issue 5, pp. 503-513, 2019.
Evolutionary classification of CRISPR-Cas systems: a burst of class 2 and derived variants; Nature Reviews Microbiology; Makarova, Kira S.; Wolf, Yuri I.; Iranzo, Jaime; Shmakov, Sergey A.; Alkhnbashi, Omer S.; Brouns, Stan J. J.; Charpentier, Emmanuelle; Cheng, David; Haft, Daniel H.; Horvath, Philippe; Moineau, Sylvain; Mojica, Francisco J. M.; Scott, David; Shah, Shiraz A.; Siksnys, Virginijus; Terns, Michael P.; Venclovas, Česlovas; White, Malcolm F.; Yakunin, Alexander F.; Yan, Winston; Zhang, Feng; Garrett, Roger A.; Backofen, Rolf; van der Oost, John; Barrangou, Rodolphe; Koonin, Eugene V.; Vol. 18 Issue 2, pp. 67-83, 2020.
CRISPR technologies are going to need a bigger toolbox; Nature Reviews Drug Discovery; Mullard, Asher; Vol. 20 Issue 11, pp. 808-809, 2021.
A vast potential genome editor toolbox; Nature Reviews Genetics; Otto, Grant; Vol. 22 Issue 12, p. 747, 2021.
Seeking more nucleases; Nature Methods; Tang, Lei; Vol. 19 Issue 1, p. 27, 2022.
Hypercompact adenine base editors based on a Cas12f variant guided by engineered RNA; Nature Chemical Biology, Kim, Do Yon; Chung, Yuhee; Lee, Yujin; Jeong, Dongmin; Park, Kwang-Hyun; Chin, Hyun Jung; Lee, Jeong Mi; Park, Seyeon; Ko, Sumin; Ko, Jeong-Heon; Kim, Yong-Sam; Vol. 18 Issue 9, pp. 1005-1013, 2022.
Voyage to minimal base editors; Nature Chemical Biology; Song, Beomjong; Bae, Sangsu; Vol. 18 Issue 9, pp. 920-921, 2022.
Mammalian genome innovation through transposon domestication; Nature Cell Biology; Modzelewski, Andrew J.; Gan Chong, Johnny; Wang, Ting; He, Lin; Vol. 24 Issue 9, pp. 1332-1340, 2022.
Recent Advances in CRISPR-Cas Technologies for Synthetic Biology; Journal of Microbiology; Jeong, Song Hee; Lee, Ho Joung; Lee, Sang Jun; Vol. 61 Issue 1, pp. 13-36, 2023.

In addition, the TnpBs contemplated herein for use in the delivery systems (e.g., LNPs) and methods described herein include TnpB proteins described in the patent literature and/or which are otherwise available in the art. For example, any of the TnpB proteins disclosed in the following references may be used in the delivery compositions (e.g., LNP compositions) and methods of the present disclosure: WO 2016/205711 A1; WO 2016/205749 A1; WO 2016/205749 A9; WO 2016/205764 A1; WO 2016/205764 A9; WO 2017/117395 A1; WO 2018/035250 A1; WO 2019/068011 A2; WO 2019/089808 A1; WO 2019/089820 A1; WO 2019/090173 A1; WO 2019/090174 A1WO 2019/090175 A1WO 2019/178428 A1WO 2020/131862 A1WO 2020/181101 A1; WO 2020/207560 A1; WO 2020/247882 A1; WO 2021/050593 A1; WO 2021/050601 A1; WO 2021/102042 A1; WO 2021/113763 A1; WO 2021/113769 A1; WO 2021/119006 A1; WO 2021/159020 A2; WO 2021/183807 A1; WO 2021/188286 A2; WO 2021/188729 A1; WO 2021/202568 A1; WO 2021/247924 A1; WO 2021/257997 A2; WO 2022/076425 A1; WO 2022/076890 A1; WO 2022/086846 A2; WO 2022/087494 A1; WO 2022/098923 A1; WO 2022/140572 A1; WO 2022/150651 A1; WO 2022/159892 A1; WO 2022/173830 A1; WO 2022/174144 A1; WO 2022/248607 A2; WO 2022/253903 A1; WO 2023/004430 A1; WO 2023/015259 A2; WO 2023/028444 A1; WO 2023/039436 A1; WO 2023/039491 A2; WO 2023/069790 A1; WO 2023/069972 A1; WO 2023/091696 A1; WO 2023/091884 A1; WO 2023/091888 A2; WO 2023/097224 A1; WO 2023/097228 A1; WO 2023/097282 A1; WO 2023/275601 A1; WO 2023/282597 A1.

The TnpB editing systems of the present disclosure may also include one or more TnpB polypeptides from the Table A, or a polypeptide having at least 70%, 75%, 80%, 85%, 90%, 95%, 99%, or 100% sequence identity with one or more of the TnpB polypeptides of Table A.

In certain example embodiments, the TnpB polypeptides are between 175 and 800 amino acids in size, between 200 and 790 amino acids in size, between 200 and 780 amino acids in size, between 200 and 770 amino acids in size, between 200 and 760 amino acids in size, between 200 and 750 amino acids in size, between 200 and 740 amino acids in size, between 200 and 730 amino acids in size, between 200 and 720 amino acids in size, between 200 and 720 amino acids in size, between 200 and 710 amino acids in size, between 200 and 700 amino acids in size, between 200 and 690 amino acids in size, between 200 and 680 amino acids in size, between 200 and 670 amino acids in size, between 200 and 660 amino acids in size, between 200 and 650 amino acids in size, between 200 and 640 amino acids in size, between 200 and 630 amino acids in size, between 200 and 620 amino acids in size, between 200 and 610 amino acids in size, between 200 and 600 amino acids in size, between 200 and 590 amino acids in size, between 200 and 580 amino acids in size, between 200 and 570 amino acids in size, between 200 and 560 amino acid, between 200 between 550 amino acids, between 200 and 540 amino acids, between 200 and 530 amino acids, between 200 and 520 amino acids, between 200 and 510 amino acids, between 200 and 500 amino acids, between 200 and 490 amino acids, between 200 and 480 amino acids, between 200 and 470 amino acids, between 200 and 460 amino acids, between 200 and 450 amino acids, between 200 and 440 amino acids, between 200 and 430 amino acids, between 200 and 420 amino acids, between 200 and 410 amino acids, between 210 and 500 amino acids, between 220 and 500 amino acids. Between 230 and 500 amino acids, between 240 and 500 amino acids, between 250 and 500 amino acids, between 260 and 500 amino acids, between 270 and 500 amino acids, between 280 and 500 amino acids, between 290 and 500 amino acids, between 300 and 500 amino acids, between 250 and 490 amino acids, between 250 and 480 amino acids, between 250 and 490 amino acids, or between 250 and 600 amino acids. In one embodiment, the TnpB polypeptide is between 300 and 500 amino acids, or between 350 and 450 amino acids.

In one embodiment, the TnpB polypeptides may comprise a modified naturally occurring protein, functional fragment or truncated version thereof, or a non-naturally occurring protein. In one embodiment, the TnpB polypeptide comprises one or more domains originating from other TnpB polypeptides, more particularly originating from different organisms. In one embodiment, the TnpB polypeptides may be designed by in silico approaches. Examples of in silico protein design have been described in the art and are therefore known to a skilled person.

The TnpB polypeptides also encompasses homologs or orthologs of TnpB polypeptides whose sequences are specifically described herein (such as the sequences of Table A). The terms “ortholog” and “homolog” are well known in the art. By means of further guidance, a “homolog” of a protein as used herein is a protein of the same species which performs the same or a similar function as the protein it is a homolog of. Homologous proteins may but need not be structurally related, or are only partially structurally related. An “ortholog” of a protein as used herein is a protein of a different species which performs the same or a similar function as the protein it is an orthologue of. Orthologous proteins may be, but may not always be, structurally related or are only partially structurally related. In particular embodiments, the homolog or ortholog of a TnpB polypeptide such as referred to herein has a sequence homology or identity of at least 80%, at least 81%, at least 82%, at least 83%, at least 84% at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% with a TnpB polypeptide, more specifically with a TnpB sequence identified in Table A. In particular embodiments, a homolog or ortholog is identified according to its domain structure and/or function. Sequence alignments as well as folding studies and domain predictions can aid in the identification of a homolog or ortholog with the structural and functional characteristics identifying TnpB polypeptides, particularly those with conserved residues, including catalytic residues, and domains of TnpB polypeptides.

In one embodiment, the TnpB polypeptide comprises at least at least one RuvC-like nuclease domain. The RuvC domain may comprise conserved catalytic amino acids indicative of the RuvC catalytic residue. In an example embodiment, the RuvC catalytic residue may be referenced relative to D191, E278, and D361 of the TnpB of D. radiodurans or a corresponding amino acid in an aligned sequence. In an aspect, the RuvC domain may comprise multiple subdomains, e.g., RuvC-I, RuvC-II and RuvC-III. The subdomains may be separated by intervening amino acid sequence of the protein.

In one embodiment, examples of the RuvC domain include any polypeptides a structural similarity and/or sequence similarity to a RuvC domain described in the art. In some examples, the RuvC domain may have an amino acid sequence that share at least 50%, at least 55%, at least 60%, at least 65%, at least 70%, at least 75%, at least 80%, at least 85%, at least 90%, at least 95%, at least 99%, or 100% sequence identity with RuvC domains known in the art. One of ordinary skill in the art can modify, substitute, or otherwise alter the activity of the RuvC domain to alter the nuclease activity, such as whether and/or where the nuclease cuts the DNA.

In embodiments, the TnpB polypeptide has a nuclease activity. In one embodiment, the TnpB and the targeting RNA (e.g., the reRNA) can direct sequence-specific nuclease activity. The cleavage may result in a 5′ overhang. The cleavage may occur distal to a target-adjacent motif (TAM), and may occur at the site of the spacer (i.e., the spacer of the reRNA which is complementary to the target sequences) annealing site or 3′ of the target sequence. In an aspect, the TnpB cleaves at multiple positions within and beyond the nucleic acid component annealing site. In an aspect, DNA cleavage occurs 15, 16, 17, 18, 19, 20, 21, 22, 23, 24, 25 or more base pairs distal to the TAM and results in a 5′ overhang. In various embodiments, the TnpB has a nuclease activity against single-stranded DNA. In other embodiments, the TnpB has a nuclease activity against double-stranded DNA.

### TnpB Modifications

In various aspects, the present invention provides one or more modifications of TnpB comprising TnpB fusions, TnpB mutations to increase sufficiency and/or efficiency and modification of TnpB reRNA. In some embodiments, one or more domains of the TnpB are modified, e.g., wedge domain, corresponding to the β-barrel, REC—helical bundle, RuvC —RuvC domain with the inserted helical hairpin (HH) and the zinc-finger domain (ZnF).

Without intending to be limited to any particular theory, TnpB operates as a homodimer with one DNA molecule and for some orthologs, its ability to form this conformation may be efficacy limiting. Takeda, Satoru N et al. “Structure of the miniature type V-F CRISPR-Cas effector enzyme.” Molecular cell vol. 81,3 (2021): 558-570.e3. doi: 10.1016/j.molcel.2020.11.035

Karvelis et al. demonstrated Deinococcus radiodurans ISDra2 TnpB to be an RNA-directed nuclease guided by RE-derived RNA (reRNA) to cleave DNA next to the 5′ TTGAT transposon associated motif (TAM). Karvelis, T., Druteika, G., Bigelyte, G. et al. Transposon-associated TnpB is a programmable RNA-guided DNA endonuclease. Nature 599, 692-696 (2021)(the contents of which are incorporated herein by reference in their entirety)

Without being bound by theory, it is contemplated that TnpB likely operates as a homodimer. Recent studies show that Cas9-Cas9 fusions displayed higher levels of genome modification and a higher proportion of these editing events were precise deletions than are observed for two independent Cas9 nucleases. Bolukbasi, M. F., Liu, P., Luk, K. et al. Orthogonal Cas9-Cas9 chimeras provide a versatile platform for genome editing. Nat Commun 9, 4856 (2018).

Accordingly, in one embodiment, a TnpB is fused to a second TnpB or the like, for example TnpB-TnpB or TnpB-Cas9. Such dual-nuclease formats comprise one TnpB component displaying expanded targeting and/or enhanced specificity and the second TnpB component having nuclease activity. In other preferred embodiments, a TnpB is fused to two or more nuclease proteins.

The TnpB polypeptide may comprise one or more modifications. As used herein, the term “modified” with regard to a TnpB polypeptide generally refers to a TnpB polypeptide having one or more modifications or mutations (including point mutations, truncations, insertions, deletions, chimeras, fusion proteins, etc.) compared to the wild type counterpart from which it is derived (e.g., from a TnpB sequence from Tables B or C). By derived is meant that the derived enzyme is largely based, in the sense of having a high degree of sequence or structural homology with, a wildtype enzyme, but that it has been mutated (modified) in some way as known in the art or as described herein.

The modified proteins, e.g., modified TnpB polypeptide may be catalytically inactive (dead). As used herein, a catalytically inactive or dead nuclease may have reduced, or no nuclease activity compared to a wildtype counterpart nuclease. In some cases, a catalytically inactive or dead nuclease may have nickase activity. In some cases, a catalytically inactive or dead nuclease may not have nickase activity. Such a catalytically inactive or dead nuclease may not make either double-strand or single-strand break on a target polynucleotide but may still bind or otherwise form complex with the target polynucleotide.

It will be appreciated that TnpB nickase can be prepared by engineering TnpB variants having corresponding mutations/substitutions to those in Cas12a nickase enzymes, such as those described in Murugan K, Seetharam A S, Severin A J, Sashital D G. CRISPR-Cas12a has widespread off-target and dsDNA-nicking effects. J Biol Chem. 2020 Apr. 24;295 (17): 5538-5553; Bijoya Paul and others, Mechanics of CRISPR-Cas12a and engineered variants on A-DNA, Nucleic Acids Research, Volume 50, Issue 9, 20 May 2022, Pages 5208-5225, each of which are incorporated herein by reference.

In an embodiment, eukaryotic homologues of bacterial TnpB may be utilized in the present invention. These TnpB-like proteins, Fanzor 1 and Fanzor 2, while having a shared amino acid motif in their C-terminal half regions, are variable in their N terminal regions.

In one embodiment, the modifications of the TnpB polypeptide may or may not cause an altered functionality. By means of example, modifications which do not result in an altered functionality include for instance codon optimization for expression into a particular host, or providing the nuclease with a particular marker (e.g. for visualization). Modifications with may result in altered functionality may also include mutations, including point mutations, insertions, deletions, truncations (including split nucleases), etc., as well as chimeric nucleases (e.g., comprising domains from different orthologues or homologues) or fusion proteins. Fusion proteins may without limitation include, for instance, fusions with heterologous domains or functional accessory domains (e.g., localization signals, catalytic domains, etc.). In one embodiment, various different modifications may be combined (e.g., a mutated nuclease which is catalytically inactive and which further is fused to a functional domain, such as for instance to induce DNA methylation or another nucleic acid modification, such as including without limitation, a break (e.g. by a different nuclease (domain)), a mutation, a deletion, an insertion, a replacement, a ligation, a digestion, a break or a recombination). As used herein, “altered functionality” includes without limitation an altered specificity (e.g., altered target recognition, increased (e.g., “enhanced” TnpB polypeptide) or decreased specificity, or altered TAM recognition), altered activity (e.g., increased or decreased catalytic activity, including catalytically inactive nucleases or nickases), and/or altered stability (e.g., fusions with destabilization domains).

Examples of all these modifications are known in the art. It will be understood that a “modified” nuclease as referred to herein, and in particular a “modified” TnpB polypeptide or system or complex preferably still has the capacity to interact with or bind to the polynucleic acid (e.g., in complex with the nucleic acid component molecule). Such modified TnpB polypeptide can be combined with the deaminase protein or active domain thereof as described herein.

In one embodiment, an unmodified TnpB polypeptide may have cleavage activity. In one embodiment, the TnpB polypeptides may direct cleavage of one or both nucleic acid (DNA or RNA) strands at the location of or near a target sequence, such as within the target sequence and/or within the complement of the target sequence or at sequences associated with the target sequence. In one embodiment, the TnpB polypeptides may direct cleavage of one or both DNA or RNA strands within about 1, 2, 3, 4, 5, 6, 7, 8, 9, 10, 15, 20, 25, 50, 100, 200, 500, or more base pairs or nucleotides from the first or last nucleotide of a target sequence. In one embodiment, the cleavage may be staggered, i.e., generating sticky ends. In one embodiment, the cleavage is a staggered cut with a 5′ overhang. In one embodiment, the cleavage is a staggered cut with a 5′ overhang of 1 to 5 or up to 10 nucleotides. In particular embodiments, the TnpB polypeptides cleave DNA strands.

In one embodiment, a TnpB polypeptide may be mutated with respect to a corresponding wild-type enzyme (e.g., the TnpB polypeptides of Table A) such that the mutated TnpB lacks the ability to cleave one or both strands of a target polynucleotide containing a target sequence. As a further example, two or more catalytic domains of a TnpB polypeptide (e.g., RuvC) may be mutated to produce a mutated TnpB polypeptide substantially lacking all DNA cleavage activity. In one embodiment, a TnpB polypeptide may be considered to substantially lack all polynucleotide cleavage activity when the polynucleotide cleavage activity of the mutated enzyme is no more than 25%, no more than 10%, no more than 5%, no more than 1%, no more than 0.1%, no more than 0.01% of the nucleic acid cleavage activity of the non-mutated form of the enzyme; an example can be when the nucleic acid cleavage activity of the mutated form is nil or negligible as compared with the non-mutated form.

In one embodiment, the TnpB polypeptide may comprise one or more modifications resulting in enhanced activity and/or specificity, such as including mutating residues that stabilize the targeted or non-targeted strand. In one embodiment, the altered or modified activity of the engineered TnpB polypeptide comprises increased targeting efficiency or decreased off-target binding. In one embodiment, the altered activity of the engineered TnpB polypeptide comprises modified cleavage activity. In one embodiment, the altered activity comprises increased cleavage activity as to the target polynucleotide loci. In one embodiment, the altered activity comprises decreased cleavage activity as to the target polynucleotide loci. In one embodiment, the altered activity comprises decreased cleavage activity as to off-target polynucleotide loci. In one embodiment, the modified nuclease comprises a modification that alters association of the protein with the nucleic acid molecule comprising RNA, or a strand of the target polynucleotide loci, or a strand of off-target polynucleotide loci.

In an aspect of the invention, the engineered TnpB polypeptide comprises a modification that alters formation of the TnpB polypeptide and related complex. In one embodiment, the altered activity comprises increased cleavage activity as to off-target polynucleotide loci. Accordingly, in one embodiment, there is increased specificity for target polynucleotide loci as compared to off-target polynucleotide loci. In other embodiments, there is reduced specificity for target polynucleotide loci as compared to off-target polynucleotide loci. In one embodiment, the mutations result in decreased off-target effects (e.g. cleavage or binding properties, activity, or kinetics), such as in case for TnpB polypeptide for instance resulting in a lower tolerance for mismatches between target and the reRNA. Other mutations may lead to increased off-target effects (e.g., cleavage or binding properties, activity, or kinetics). Other mutations may lead to increased or decreased on-target effects (e.g., cleavage or binding properties, activity, or kinetics). In one embodiment, the mutations result in altered (e.g., increased or decreased) activity, association or formation of the functional nuclease complex. Examples mutations include mutation of negative or neutral residues to positively charged residues, or positively charged residues to neutral or neutral residues to negative residues and/or (evolutionary) conserved residues, such as conserved positively charged residues, in order to enhance specificity. In one embodiment, such residues may be mutated to uncharged residues, such as alanine. Because the TnpB polypeptide interacts with guide or bound DNA over the length of the TnpB polypeptide, mutation of residues across the TnpB polypeptide may be utilized for altered activity. In an aspect, the TnpB polypeptide residues for mutation are altered based on amino acid sequence positions of Deinococcus radiodurans ISDra2, see, e.g. Karvelis et al., Nature 599, 692-696 (2021).

Preferably, one or more TnpB comprises one or more mutated residues in the Rec domain and optionally these mutated residues are hydrophobic. Alternatively, one or more TnpB comprises mutated residues in the RuvC domain. Preferably, one or more of the mutated residues typically form a hydrogen bond with another TnpB monomer. More preferably, a combination of the two sets of mutations as described above.

In yet other embodiments, the TnpB-nuclease fusions are linked using a polypeptide comprising glycine and serine residues or unstructured XTEN protein polymer.

In other exemplary embodiments, the TnpB-nuclease fusions are linked using an RNA wherein the RNA comprises a guide RNA or a reRNA.

In further embodiments, the TnpB-nuclease fusions comprise one or more nuclear localization signals selected from but not limited to SV40, c-Myc, NLP-1.

Also described herein are methods and compositions for increasing the TnpB-mediated editing efficiency. In some aspects, the editing effiency is greater than 70%, at least 70.5%, at least 71%, at least 72%, at least 73%, at least 74%, at least 75%, at least 76%, at least 77%, at least 78%, at least 79%, at least 80%, at least 85%, at least 90%, at least 95%, or at least 99%.

Additionally described herein are methods and compositions for increasing the TnpB-mediated editing specificity. In some aspects, the editing specificity is greater than 70%, at least 70.5%, at least 71%, at least 72%, at least 73%, at least 74%, at least 75%, at least 76%, at least 77%, at least 78%, at least 79%, at least 80%, at least 85%, at least 90%, at least 95%, or at least 99%.

### TnpB Editor Formats

In other aspect, the TnpB-based genome editing systems may comprise one or more accessory proteins having genome modifying functions, including recombinases, invertases, nucleases, polymerases, ligases, deaminases, or reverse transcriptases. In various embodiments, the accessory proteins may be provided separately. In other embodiments, the accessory proteins may be fused to TnpB, optionally with a linker. In various embodiments, TnpB and depending on the accessory function involved, a TnpB protein may be combined with one or more accessory functions to produce a multi-functional editing system. For example, as described further herein, TnpB may be coupled with a deaminase to form a base editing system. In another example, a TnpB may be coupled with a reverse transcriptase to form a prime editing system.

In one embodiment, the accessory function that is added or otherwise coupled or attached to a TnpB polypeptide (e.g., deaminase or reverse transcriptase) provides for a TnpB-based system that is capable of performing a specialized function or activity (e.g., base editing or prime editing). For example, the TnpB protein may be fused, operably coupled to, or otherwise associated with one or more heterologous functionals domains. In certain example embodiments, the TnpB protein may be a catalytically dead TnpB protein and/or have nickase activity. A nickase is an TnpB protein that cuts only one strand of a double stranded target. In such embodiments, the catalytically inactive TnpB or nickase provide a sequence specific targeting functionality via the coRNA that delivers the functional domain to or proximate a target sequence.

It is also contemplated that the TnpB complex as a whole may be associated with two or more functional domains. For example, there may be two or more functional domains associated with the TnpB polypeptide, or there may be two or more functional domains associated with the reRNA component (via one or more adaptor proteins or aptamers), or there may be one or more functional domains associated with the TnpB polypeptide and one or more functional domains associated with the reRNA component.

In one embodiment, one or more functional domains are associated with a TnpB polypeptide via an adaptor protein, for example as used with the modified guides of Konnerman et al. (Nature 517, 583-588, 29 Jan. 2015). In one embodiment, the one or more functional domains is attached to the adaptor protein so that upon binding of the TnpB polypeptide to reRNA and target, the functional domain is in a spatial orientation allowing for the functional domain to function in its attributed function.

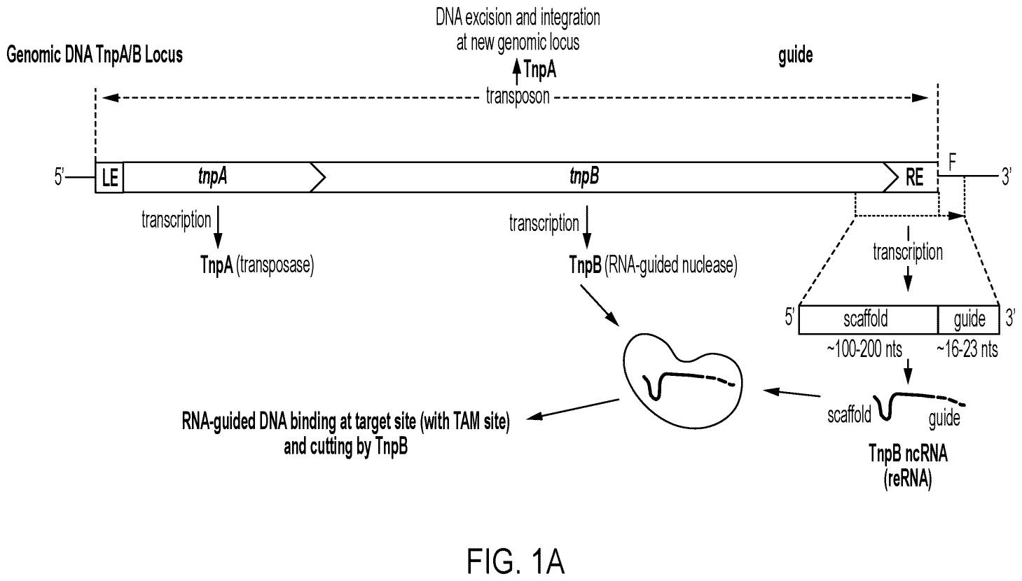

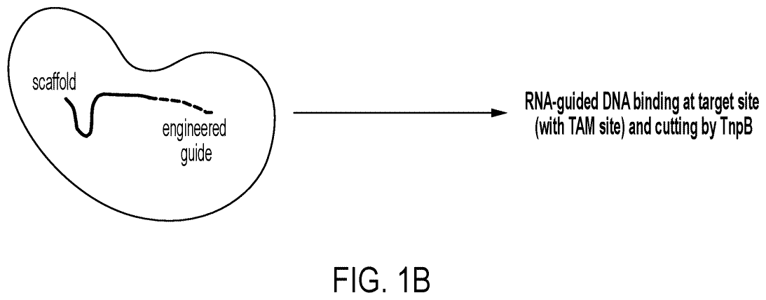

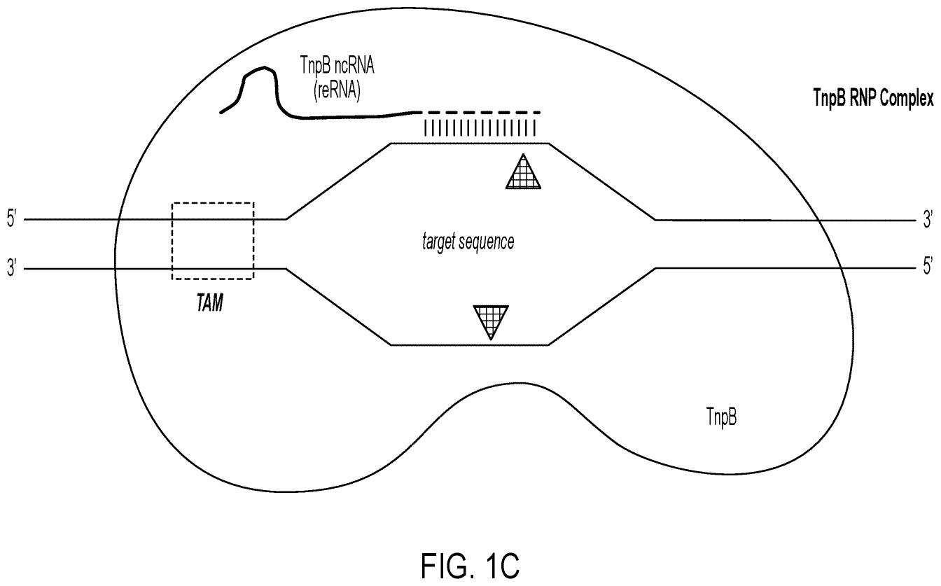

Exemplary functional accessory domains that may be fused to, operably coupled to, or otherwise associated with an TnpB protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, a ligase domain, a topoisomerase domain, a deaminase domain, a polymerase domain (e.g., reverse transcriptase), an integrase domain, and combinations thereof. In an embodiment, the functional domain is an HNH domain, and may be used with a naturally catalytically inactive TnpB protein to engineer a nickase. Methods for generating catalytically dead TnpB or a nickase TnpB can be adapted from approaches in Cas9 proteins, see, for example, WO 2014/204725, Ran et al. Cell. 2013 September 12; 154 (6): 1380-1389, known in the art and incorporated herein by reference. Briefly, one or more mutations in the catalytic domain of the RuvC domain and/or the HNH domain of the TnpB protein can be introduced that may reduce or abolish NHEJ activity. In an aspect, at least one mutation in the RuvC domain and at least one mutation in the HNH domain is provided. In an embodiment, the TnpB polypeptide comprises a mutation at D191 and/or E278 based on amino acid sequence positions of Deinococcus radiodurans ISDra2 (see FIG. 1). In an aspect, the amino acid mutations comprise D191A and/or E278A based on amino acid sequence positions of Deinococcus radiodurans ISDra2.

In one embodiment, the functional domains can have one or more of the following activities: nucleobase deaminse activity, reverse transcriptase activity, retrotransposase activity, transposase activity, integrase activity, recombinase activity, topoisomerase activity, ligase activity, polymerase activity, helicase activity, methylase activity, demethylase activity, translation activation activity, translation initiation activity, translation repression activity, transcription activation activity, transcription repression activity, transcription release factor activity, histone modification activity, nuclease activity (e.g. VirD2), single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In one embodiment, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) betagalactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the TnpB protein. In embodiments having two or more functional domains, each of the two can be positioned at or near or in proximity to a terminus of the TnpB protein. In one embodiment, such as those where the functional domain is operably coupled to the effector protein, the one or more functional domains can be tethered or linked via a suitable linker (including, but not limited to, GlySer linkers) to the TnpB protein. When there is more than one functional domain, the functional domains can be same or different. In one embodiment, all the functional domains are the same. In one embodiment, all of the functional domains are different from each other. In one embodiment, at least two of the functional domains are different from each other. In one embodiment, at least two of the functional domains are the same as each other.

### TnpB Base Editors

In other embodiments, the TnpB-based genome editing systems contemplated herein may be in the format of a base editor wherein a TnpB nuclease is substituted in place of a Cas9 nuclease. Any of the delivery systems described herein—including LNPs—may be used to deliver a TnpB base editing system. Base editors are generally composed of an engineered deaminase and a catalytically impaired CRISPR-Cas9 variant and enzymatically convert one base to another base at a specific target site with the assistance of endogenous DNA repair systems in the cell.

Base editing was first described in Komor et al., “Programmable editing of a target base in genomic DNA without double-stranded DNA cleavage,” Nature, May 19, 2016, 533 (7603); pp. 420-424 in the form of cytosine base editors or CBEs followed by the disclosure of Gaudelli et al., “Programmable base editing of A-T to G-C in genomic DNA without DNA cleavage,” Nature, Vol. 551, pp. 464-471 describing adenine base editors or ABEs. Subsequently, base editing has been described in numerous scientific publications, including, but not limited to (i) Kim J S. Precision genome engineering through adenine and cytosine base editing. Nat Plants. 2018 March;4 (3): 148-151. doi: 10.1038/s41477-018-0115-z. Epub 2018 Feb. 26. PMID: 29483683.; (ii) Wei Y, Zhang X H, Li D L. The “new favorite” of gene editing technology-single base editors. Yi Chuan. 2017 Dec. 20;39 (12): 1115-1121. doi: 10.16288/j.yczz.17-389. PMID: 29258982; (iii) Tang J, Lee T, Sun T. Single-nucleotide editing: From principle, optimization to application. Hum Mutat. 2019 December;40 (12): 2171-2183. doi: 10.1002/humu.23819. Epub 2019 Sep. 15. PMID: 31131955; PMCID: PMC6874907; (iv) Grünewald J, Zhou R, Lareau C A, Garcia S P, Iyer S, Miller B R, Langner L M, Hsu J Y, Aryee M J, Joung J K. A dual-deaminase CRISPR base editor enables concurrent adenine and cytosine editing. Nat Biotechnol. 2020 July;38 (7): 861-864. doi: 10.1038/s41587-020-0535-y. Epub 2020 Jun. 1. PMID: 32483364; PMCID: PMC7723518; (v) Sakata R C, Ishiguro S, Mori H, Tanaka M, Tatsuno K, Ueda H, Yamamoto S, Seki M, Masuyama N, Nishida K, Nishimasu H, Arakawa K, Kondo A, Nureki O, Tomita M, Aburatani H, Yachie N. Base editors for simultaneous introduction of C-to-T and A-to-G mutations. Nat Biotechnol. 2020 July;38 (7): 865-869. doi: 10.1038/s41587-020-0509-0. Epub 2020 Jun. 2. Erratum in: Nat Biotechnol. 2020 Jun. 5;: PMID: 32483365; (vi) Fan J, Ding Y, Ren C, Song Z, Yuan J, Chen Q, Du C, Li C, Wang X, Shu W. Cytosine and adenine deaminase base-editors induce broad and nonspecific changes in gene expression and splicing. Commun Biol. 2021 Jul. 16;4 (1): 882. doi: 10.1038/s42003-021-02406-5. PMID: 34272468; PMCID: PMC8285404; (vii) Zhang S, Yuan B, Cao J, Song L, Chen J, Qiu J, Qiu Z, Zhao X M, Chen J, Cheng T L. TadA orthologs enable both cytosine and adenine editing of base editors. Nat Commun. 2023 Jan. 26;14 (1): 414. doi: 10.1038/s41467-023-36003-3. PMID: 36702837; PMCID: PMC988000; and (viii) Zhang S, Song L, Yuan B, Zhang C, Cao J, Chen J, Qiu J, Tai Y, Chen J, Qiu Z, Zhao X M, Cheng T L. TadA reprogramming to generate potent miniature base editors with high precision. Nat Commun. 2023 Jan. 26;14 (1): 413. doi: 10.1038/s41467-023-36004-2. PMID: 36702845; PMCID: PMC987999, each of which are incorporated herein by reference in their entireties.

Amino acid and nucleotide sequences of base editor deaminases-including adenosine and cytidine deaminases, are readily available in the art. For example, exemplary deaminases can be found in the following published patent applications, each of their contents (including any and all biological sequences) are incorporated herein by reference:

| | |
|---|---|
| US 2023/0021641 A1 | CAS9 VARIANTS HAVING NON-CANONICAL PAM |
| | SPECIFICITIES AND USES THEREOF |
| U.S. Pat. No. 11,542,496 B2 | Cytosine to guanine base editor |
| U.S. Pat. No. 11,542,509 B2 | Incorporation of unnatural amino acids into proteins using base editing |
| US 2022/0315906 A1 | BASE EDITORS WITH DIVERSIFIED TARGETING SCOPE |
| US 2022/0282275 A1 | G-TO-T BASE EDITORS AND USES THEREOF |
| US 2022/0249697 A1 | AAV DELIVERY OF NUCLEOBASE EDITORS |

In some embodiments, the disclosure provides a TnpB base editing system or a polynucleotide encoding a TnpB base editing system that may be delivered by any of the delivery systems disclosed herein, include LNPs. In some embodiments, the delivery system may comprise a component of a TnpB base editing system or a polynucleotide (DNA or RNA) encoding a component of a base editing system. Such components may include a TnpB protein, a deaminase (optionally fused to the TnpB protein), and a TnpB ncRNA sequence.

Base editing does not require double-stranded DNA breaks or a DNA donor template. In some embodiments, base editing comprises creating an SSB in a target double-stranded DNA sequence and then converting a nucleobase. In some embodiments, the nucleobase conversion is an adenosine to a guanine. In some embodiments, the nucleobase conversion is a thymine to a cytosine. In some embodiments, the nucleobase conversion is a cytosine to a thymine. In some embodiments, the nucleobase conversion is a guanine to an adenosine. In some embodiments, the nucleobase conversion is an adenosine to inosine. In some embodiments, the nucleobase conversion is a cytosine to uracil.

A base editing system comprises a base editor which can convert a nucleobase. The base editor (“BE”) comprises a partially inactive TnpB protein which is connected to a deaminase that precisely and permanently edits a target nucleobase in a polynucleotide sequence. A base editor comprises a polynucleotide programmable nucleotide binding domain and a nucleobase editing domain (e.g., adenosine deaminase or cytosine deaminase). In some embodiments, the partially inactive TnpB protein is a TnpB nickase (i.e., cuts only a single strand).

A variety of nucleobase modifying enzymes are suitable for use in the nucleobase systems disclosed herein. In some embodiments, the nucleobase modifying enzyme is a RNA base editor. In some embodiments, the RNA base editor can be a cytidine deaminase, which converts cytidine into uridine. Non-limiting examples of cytidine deaminases include cytidine deaminase 1 (CDA1), cytidine deaminase 2 (CDA2), activation-induced cytidine deaminase (AICDA), apolipoprotein B mRNA-editing complex (APOBEC) family cytidine deaminase (e.g., APOBEC1, APOBEC2, APOBEC3A, APOBEC3B, APOBEC3C, APOBEC3D/E, APOBEC3F, APOBEC3G, APOBEC3H, APOBEC4), APOBEC1 complementation factor/APOBEC1 stimulating factor (ACF1/ASF) cytidine deaminase, cytosine deaminase acting on RNA (CDAR), bacterial long isoform cytidine deaminase (CDDL), and cytosine deaminase acting on tRNA (CDAT). In other embodiments, the RNA base editor can be an adenosine deaminase, which converts adenosine into inosine, which is read by polymerase enzymes as guanosine. In certain embodiments, adenosine deaminases include tRNA adenine deaminase, adenosine deaminase, adenosine deaminase acting on RNA (ADAR), and adenosine deaminase acting on tRNA (ADAT).

In some embodiments, in the nucleobase editing systems disclosed herein, the Cas effector may associate with one or more functional domains (e.g., via fusion protein or suitable linkers). In some embodiments, the effector domain comprises one or more cytidine or nucleotide deaminases that mediate editing of via hydrolytic deamination. In certain embodiments, the effector domain comprises the adenosine deaminase acting on RNA (ADAR) family of enzymes. In certain embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu) ADAR1 or (hu) ADAR2, preferably huADAR2 or catalytic domain thereof.

In some embodiments, the cytidine deaminase is a human, rat or lamprey cytidine deaminase. In some embodiments, the cytidine deaminase is an apolipoprotein B mRNA-editing complex (APOBEC) family deaminase, an activation-induced deaminase (AID), or a cytidine deaminase 1 (CDA1).

In certain embodiments, the adenosine deaminase is adenosine deaminase acting on RNA (ADAR). In certain embodiments, the ADAR is ADAR (ADAR1), ADARB1 (ADAR2) or ADARB2 (ADAR3)(see, e.g., Savva et al. Genon. Biol. 2012, 13 (12): 252).

In some embodiments, the gene editing system comprises AID/APOBEC (apolipoprotein B editing complex) family of enzymes deaminates cytidine to uridine, leading to mutations in RNA and DNA.

In some embodiments, the nucleobase editing system comprises ADAR and an antisense oligonucleotide. In certain embodiments, the antisense oligonucleotide is chemically optimized antisense oligonucleotide. In certain embodiments, the antisense oligonucleotide is administered for the nucleobase editing, wherein the antisense oligonucleotide activates human endogenous ADAR for nucleobase editing. Such ADAR and antisense oligonucleotide editing system provides a safer site-directed RNA editing with low off-target effect. See, e.g., Merkle et al., Nature Biotechnology, 2019, 37, 133-138.

Accordingly, in various aspects of the invention, the TnpB is fused to a deaminase suitable for base editing. In some embodiments, the deaminase is selected from an adenosine deaminase, E. coli tRNA adenosine, or TadA deaminase wherein TadA is engineered for higher efficiency in human cells in comparison to pWT TadA base editor. In certain embodiments, TadA is engineered through directed evolution.

In certain other embodiments, the deaminase comprises a cytidine deaminase. Preferably, the cytidine deaminase is engineered for higher efficiency in human cells in comparison to wild type cytidine deaminase base editor. In further embodiments, the TnpB genome editing system contains one or more uracil glycosylase inhibitor.

In yet other embodiments, the TnpB-deaminase fusions are linked using a polypeptide comprising glycine and serine residues or unstructured XTEN protein polymer.

In further embodiments, the TnpB RuvC domain is mutated wherein the mutation slows cleavage of the target strand or slows the cleavage of the non-target strand. In other embodiments, the TnpB is mutated to be catalytically inactive.

In certain preferred embodiments one or more deaminase is fused to a TnpB dimer. In certain embodiments, the deaminase is fused to the N-terminus of TnpB. In other embodiments, the deaminase is fused to the C-terminus of TnpB. In further embodiments, the deaminase is placed in various locations of the TnpB including without limitations: inside the Rec-domain of the TnpB, after the Rec-domain of the TnpB, in the Wedge domain of TnpB, after the Wedge domain of TnpB, in the RuvC domain of TnpB, after the RuvC domain of TnpB, in the Helical hairpin domain of TnpB, after the Helical hairpin domain of TnpB, in the ZnF domain of TnpB, after the Znf domain of TnpB. The present invention contemplates placement of the deaminase in and around or near or adjacent to the aforementioned domains.

In certain alternative embodiments, the TnpB fusion protein is co-expressed with one or more TnpB not fused to a deaminase. In other embodiments, the unfused TnpB is mutated to be catalytically inactive. In other examples, the TnpB fusion contains one or more nuclear localization signals selected or derived from SV40, c-Myc or NLP-1.

In other exemplary embodiments, the TnpB-deaminase fusions bind to a guide RNA or a reRNA. In instances where the TnpB system is fused to a polypeptide that modulates host-repair. In some examples, the polypeptide is a uracil glycosylase inhibitor. In other examples, the polypeptide inhibits mismatch repair wherein the MMR inhibiting polypeptide is a dominant negative MLH1.

### TnpB CBE

In some embodiments, the deliverable TnpB base editors may comprise a deaminase domain that is a cytidine deaminase domain. A cytidine deaminase domain may also be referred to interchangeably as a cytosine deaminase domain. In some embodiments, the cytidine deaminase catalyzes the hydrolytic deamination of cytidine (C) or deoxycytidine (dC) to uridine (U) or deoxyuridine (dU), respectively. In some embodiments, the cytidine deaminase domain catalyzes the hydrolytic deamination of cytosine (C) to uracil (U). In some embodiments, the cytidine deaminase catalyzes the hydrolytic deamination of cytidine or cytosine in deoxyribonucleic acid (DNA). Without wishing to be bound by any particular theory, fusion proteins comprising a cytidine deaminase are useful inter alia for targeted editing, referred to herein as “base editing,” of nucleic acid sequences in vitro and in vivo.

One exemplary suitable type of cytidine deaminase is a cytidine deaminase, for example, of the APOBEC family. The apolipoprotein B mRNA-editing complex (APOBEC) family of cytidine deaminase enzymes encompasses eleven proteins that serve to initiate mutagenesis in a controlled and beneficial manner (see, e.g., Conticello S G. The AID/APOBEC family of nucleic acid mutators. Genome Biol. 2008; 9 (6): 229). One family member, activation-induced cytidine deaminase (AID), is responsible for the maturation of antibodies by converting cytosines in ssDNA to uracils in a transcription-dependent, strand-biased fashion (see, e.g., Reynaud C A, et al. What role for AID: mutator, or assembler of the immunoglobulin mutasome, Nat Immunol. 2003; 4 (7): 631-638). The apolipoprotein B editing complex 3 (APOBEC3) enzyme provides protection to human cells against a certain HIV-1 strain via the deamination of cytosines in reverse-transcribed viral ssDNA (see, e.g., Bhagwat A S. DNA-cytosine deaminases: from antibody maturation to antiviral defense. DNA Repair (Amst). 2004; 3 (1): 85-89).

Some aspects of this disclosure relate to the recognition that the activity of cytidine deaminase enzymes such as APOBEC enzymes can be directed to a specific site in genomic DNA. Without wishing to be bound by any particular theory, advantages of using a nucleic acid programmable binding protein (e.g., a TnpB nuclease) as a recognition agent include (1) the sequence specificity of nucleic acid programmable binding protein (e.g., a TnpB nuclease) can be easily altered by simply changing the sgRNA sequence; and (2) the nucleic acid programmable binding protein (e.g., a TnpB nuclease) may bind to its target sequence by denaturing the dsDNA, resulting in a stretch of DNA that is single-stranded and therefore a viable substrate for the deaminase. It should be understood that other catalytic domains of napDNAbps, or catalytic domains from other nucleic acid editing proteins, can also be used to generate fusion proteins with TnpB, and that the disclosure is not limited in this regard.

In some embodiments, the cytidine deaminase is an apolipoprotein B mRNA-editing complex (APOBEC) family deaminase. In some embodiments, the cytidine deaminase is an APOBEC1 deaminase. In some embodiments, the cytidine deaminase is an APOBEC2 deaminase. In some embodiments, the cytidine deaminase is an APOBEC3 deaminase. In some embodiments, the cytidine deaminase is an APOBEC3A deaminase. In some embodiments, the cytidine deaminase is an APOBEC3B deaminase. In some embodiments, the cytidine deaminase is an APOBEC3C deaminase. In some embodiments, the cytidine deaminase is an APOBEC3D deaminase. In some embodiments, the cytidine deaminase is an APOBEC3E deaminase. In some embodiments, the cytidine deaminase is an APOBEC3F deaminase. In some embodiments, the cytidine deaminase is an APOBEC3G deaminase. In some embodiments, the cytidine deaminase is an APOBEC3H deaminase. In some embodiments, the cytidine deaminase is an APOBEC4 deaminase. In some embodiments, the cytidine deaminase is an activation-induced deaminase (AID). In some embodiments, the cytidine deaminase is a vertebrate cytidine deaminase. In some embodiments, the cytidine deaminase is an invertebrate cytidine deaminase. In some embodiments, the cytidine deaminase is a human, chimpanzee, gorilla, monkey, cow, dog, rat, or mouse deaminase. In some embodiments, the cytidine deaminase is a human cytidine deaminase. In some embodiments, the cytidine deaminase is a rat cytidine deaminase, e.g., rAPOBEC1.

In some embodiments, the nucleic acid editing domain is at least 80%, at least 85%, at least 90%, at least 92%, at least 95%, at least 96%, at least 97%, at least 98%, at least 99%, or at least 99.5% identical to any of the cytidine deaminase domain examples above.

### TnpB ABE

In other embodiments, the deliverable base editors may comprise a deaminase domain that is an adenosine deaminase domain.

The disclosure provides fusion proteins that comprise one or more adenosine deaminases fused to a TnpB nuclease. In some aspects, such fusion proteins are capable of deaminating adenosine in a nucleic acid sequence (e.g., DNA or RNA). As one example, any of the fusion proteins provided herein may be base editors, (e.g., adenine base editors). Without wishing to be bound by any particular theory, dimerization of adenosine deaminases (e.g., in cis or in trans) may improve the ability (e.g., efficiency) of the fusion protein to modify a nucleic acid base, for example to deaminate adenine. In some embodiments, any of the fusion proteins may comprise 2, 3, 4 or 5 adenosine deaminases. In some embodiments, any of the fusion proteins provided herein comprise two adenosine deaminases. Exemplary, non-limiting, embodiments of adenosine deaminases are provided herein. It should be appreciated that the mutations provided herein (e.g., mutations in ecTadA) may be applied to adenosine deaminases in other adenosine base editors, for example those provided in U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, all of which are incorporated herein by reference in their entireties.

In some embodiments, any of the adenosine deaminases provided herein is capable of deaminating adenine. In some embodiments, the adenosine deaminases provided herein are capable of deaminating adenine in a deoxyadenosine residue of DNA. The adenosine deaminase may be derived from any suitable organism (e.g., E. coli). In some embodiments, the adenosine deaminase is a naturally-occurring adenosine deaminase that includes one or more mutations corresponding to any of the mutations provided herein (e.g., mutations in ecTadA). One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

Any two or more of the adenosine deaminases described herein may be connected to one another (e.g. by a linker) within an adenosine deaminase domain of the fusion proteins provided herein. For instance, the fusion proteins provided herein may contain only two adenosine deaminases. In some embodiments, the adenosine deaminases are the same. In some embodiments, the adenosine deaminases are any of the adenosine deaminases provided herein. In some embodiments, the adenosine deaminases are different. In some embodiments, the first adenosine deaminase is any of the adenosine deaminases provided herein, and the second adenosine is any of the adenosine deaminases provided herein, but is not identical to the first adenosine deaminase. In some embodiments, the fusion protein comprises two adenosine deaminases (e.g., a first adenosine deaminase and a second adenosine deaminase). In some embodiments, the fusion protein comprises a first adenosine deaminase and a second adenosine deaminase. In some embodiments, the first adenosine deaminase is N-terminal to the second adenosine deaminase in the fusion protein. In some embodiments, the first adenosine deaminase is C-terminal to the second adenosine deaminase in the fusion protein. In some embodiments, the first adenosine deaminase and the second deaminase are fused directly or via a linker.

In some embodiments, the base editor comprises a deaminase enzyme. In some embodiments, the base editor comprises a cytidine deaminase. In some embodiments, the base editor comprises a TnpB protein fused to a cytidine deaminase enzyme. In some embodiments, the base editor comprises an adenosine deaminase. In some embodiments, the base editor comprises a TnpB protein fused to an adenosine deaminase enzyme.

In some embodiments, the base editing system comprises an uracil glycosylase inhibitor. In some embodiments, the base editing system comprises a TnpB protein fused to an uracil glycosylase inhibitor. In some embodiments, the cargo comprises an uracil glycosylase inhibitor or a polynucleotide encoding an uracil glycosylase inhibitor. In some embodiments, the cargo comprises a TnpB protein fused to an uracil glycosylase inhibitor or a polynucleotide encoding a TnpB protein fused to an uracil glycosylase inhibitor.

### TnpB Prime Editors

In various embodiments, the TnpBs may configured as a prime editing system which may be used to conduct prime editing of target nucleic acid sequences in cells, tissues, and organs in an ex vivo or in vivo manner. Such TnpB prime editing systems are deliverable by the delivery systems disclosed herein, including LNP delivery systems.

Prime editing technology is a gene editing technology that can make targeted insertions, deletions, and all transversion and transition point mutations in a target genome. Without wishing to be bound by any particular theory, the prime editing process may search and replace endogenous sequences in a target polynucleotide. The spacer sequence of a prime editing guide RNA (“PEgRNA” or “pegRNA”) recognizes and anneals with a search target sequence in a target strand of a double stranded target polynucleotide, e.g., a double stranded target DNA. A prime editing complex may generate a nick in the target DNA on the edit strand which is the complementary strand of the target strand. The prime editing complex may then use a free 3′ end formed at the nick site of the edit strand to initiate DNA synthesis, where a “primer binding site sequence” (PBS) of the PEgRNA complexes with the free 3′ end, and a single stranded DNA is synthesized (by reverse transcriptase) using an editing template of the PEgRNA as a template. As used herein, a “primer binding site” is a single-stranded portion of the PEgRNA that comprises a region of complementarity to the PAM strand (i.e., the non-target strand or the edit strand). The PBS is complementary or substantially complementary to a sequence on the PAM strand of the double stranded target DNA that is immediately upstream of the nick site.

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity (e.g., a TnpB) and a polypeptide domain having DNA polymerase activity (e.g., a reverse transcriptase). In some embodiments, the prime editor comprises a TnpB nuclease. In some embodiments, the TnpB is a fully active TnpB nuclease. In other embodiments, the TnpB is a nickase. As used herein, the term “nickase” refers to a TnpB nuclease capable of cleaving only one strand of a double-stranded DNA target. In some embodiments, the prime editor comprises a polypeptide domain that is an inactive TnpB nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide (DNA binding domain) and a reverse transcriptase polypeptide (DNA polymerase) that are derived from different species. For example, a prime editor may comprise a TnpB of Table A and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

The editing template may comprise one or more intended nucleotide edits compared to the endogenous double stranded target DNA sequence. Accordingly, the newly synthesized single stranded DNA also comprises the nucleotide edit(s) encoded by the editing template. Through removal of the editing target sequence on the edit strand of the double stranded target DNA and DNA repair mechanism, the newly synthesized single stranded DNA replaces the editing target sequence, and the desired nucleotide edit(s) are incorporated into the double stranded target DNA.

Prime editing was first described in Anzalone et al., “Search- and -replace genome editing without double-strand breaks or donor DNA,” Nature, December 2019, 576 (7789): pp. 149-157, which is incorporated herein in its entirety. Prime editing has subsequently been described and detailed in numerous follow-on publications, including, for example, (i) Liu et al., “Prime editing: a search and replace tool with versatile base changes,” Yi Chuan, Nov. 20, 2022, 44 (11): 993-1008; (ii) Lu C et al., “Prime Editing: An All-Rounder for Genome Editing. Int J Mol Sci. 2022 Aug. 30;23 (17): 9862; (iii) Velimirovic M, Zanetti L C, Shen M W, Fife J D, Lin L, Cha M, Akinci E, Barnum D, Yu T, Sherwood R I. Peptide fusion improves prime editing efficiency. Nat Commun. 2022 Jun. 18;13 (1): 3512. doi: 10.1038/s41467-022-31270-y. PMID: 35717416; PMCID: PMC9206660; (iv) Velimirovic M, Zanetti L C, Shen M W, Fife J D, Lin L, Cha M, Akinci E, Barnum D, Yu T, Sherwood R1. Peptide fusion improves prime editing efficiency. Nat Commun. 2022 Jun. 18;13 (1): 3512. doi: 10.1038/s41467-022-31270-y. PMID: 35717416; PMCID: PMC9206660; (v) Habib O, Habib G, Hwang G H, Bae S. Comprehensive analysis of prime editing outcomes in human embryonic stem cells. Nucleic Acids Res. 2022 Jan. 25;50 (2): 1187-1197. doi: 10.1093/nar/gkab1295. PMID: 35018468; PMCID: PMC8789035; (vi) Marzec M, Brąszewska-Zalewska A, Hensel G. Prime Editing: A New Way for Genome Editing. Trends Cell Biol. 2020 April;30 (4): 257-259. doi: 10.1016/j.tcb.2020.01.004. Epub 2020 Jan. 27. PMID: 32001098; (vii) Tao R, Wang Y, Jiao Y, Hu Y, Li L, Jiang L, Zhou L, Qu J, Chen Q, Yao S. Bi-PE: bi-directional priming improves CRISPR/Cas9 prime editing in mammalian cells. Nucleic Acids Res. 2022 Jun. 24;50 (11): 6423-6434. doi: 10.1093/nar/gkac506. PMID: 35687127; PMCID: PMC9226529; (viii)Nelson J W, Randolph P B, Shen S P, Everette K A, Chen P J, Anzalone A V, An M, Newby G A, Chen J C, Hsu A, Liu D R. Engineered pegRNAs improve prime editing efficiency. Nat Biotechnol. 2022 March;40 (3): 402-410. doi: 10.1038/s41587-021-01039-7. Epub 2021 Oct. 4. Erratum in: Nat Biotechnol. 2021 Dec. 8; PMID: 34608327; PMCID: PMC8930418; (ix) Doman J L, Sousa A A, Randolph P B, Chen P J, Liu D R. Designing and executing prime editing experiments in mammalian cells. Nat Protoc. 2022 November; 17 (11): 2431-2468. doi: 10.1038/s41596-022-00724-4. Epub 2022 Aug. 8. PMID: 35941224; PMCID: PMC9799714; (x) Jiao Y, Zhou L, Tao R, Wang Y, Hu Y, Jiang L, Li L, Yao S. Random-PE: an efficient integration of random sequences into mammalian genome by prime editing. Mol Biomed. 2021 Nov. 18;2 (1): 36. doi: 10.1186/s43556-021-00057-w. PMID: 35006470; PMCID: PMC8607425; and (xi) Awan MJA, Ali Z, Amin I, Mansoor S. Twin prime editor: seamless repair without damage. Trends Biotechnol. 2022 Apr;40 (4): 374-376. doi: 10.1016/j.tibtech.2022.01.013. Epub 2022 Feb. 10. PMID: 35153078, all of which are incorporated herein by reference.

In addition, prime editing has been described and disclosed in numerous published patent applications, each of which their entire contents, amino acid sequences, nucleotide sequences, and all disclosures therein are incorporated herein by reference in their entireties:

| Publication No. | Publication Date | Title |
|---|---|---|
| WO 2023/015309 A2 | Feb. 9, 2023 | IMPROVED PRIME EDITORS AND |
| | | METHODS OF USE |
| WO 2023/004439 A2 | Jan. 26, 2023 | GENOME EDITING COMPOSITIONS AND |
| | | METHODS FOR TREATMENT OF CHRONIC |
| | | GRANULOMATOUS DISEASE |
| WO 2023/288332 A2 | Jan. 19, 2023 | GENOME EDITING COMPOSITIONS AND |
| | | METHODS FOR TREATMENT OF WILSON'S |
| | | DISEASE |
| WO 2023/283092 A1 | Jan. 12, 2023 | COMPOSITIONS AND METHODS FOR |
| | | EFFICIENT GENOME EDITING |
| WO 2023/283246 A1 | Jan. 12, 2023 | MODULAR PRIME EDITOR SYSTEMS FOR |
| | | GENOME ENGINEERING |
| WO 2022/256714 A3 | Jan. 12, 2023 | GENOME EDITING COMPOSITIONS AND |
| | | METHODS FOR TREATMENT OF WILSON'S |
| | | DISEASE |
| EP 4107273 A1 | Dec. 28, 2022 | PRIME EDITING TECHNOLOGY FOR |
| | | PLANT GENOME ENGINEERING |
| WO 2022/256714 A2 | Dec. 8, 2022 | GENOME EDITING COMPOSITIONS AND |
| | | METHODS FOR TREATMENT OF WILSON'S |
| | | DISEASE |
| WO 2022/234051 A1 | Nov. 10, 2022 | SPLIT PRIME EDITING ENZYME |
| US 2022/0356469 A1 | Nov. 10, 2022 | METHODS AND COMPOSITIONS FOR |
| | | EDITING NUCLEOTIDE SEQUENCES |
| | | METHODS AND COMPOSITIONS FOR |
| | | EDITING NUCLEOTIDE SEQUENCES |
| WO 2022/206352 A1 | Oct. 6, 2022 | PRIME EDITING TOOL, FUSION RNA, AND |
| | | USE THEREOF |
| WO 2022/212926 A1 | Oct. 6, 2022 | METHODS AND COMPOSITIONS FOR |
| | | EDITING NUCLEOTIDE SEQUENCES |
| WO 2022/204476 A1 | Sep. 29, 2022 | NUCLEOTIDE EDITING TO REFRAME DMD |
| | | TRANSCRIPTS BY BASE EDITING AND |
| | | PRIME EDITING |
| WO 2022/203905 A1 | Sep. 29, 2022 | PRIME EDITING-BASED SIMULTANEOUS |
| | | GENOMIC DELETION AND INSERTION |
| U.S. Pat. No. 11,447,770 B1 | Sep. 20, 2022 | Methods and compositions for prime editing |
| | | nucleotide sequences |
| WO 2022/174829 A1 | Aug. 25, 2022 | EDITING OF DOUBLE-STRANDED DNA |
| | | WITH RELAXED PAM REQUIREMENT |
| | | FIELD OF THE DISCLOSURE |
| WO 2022/170058 A1 | Aug. 11, 2022 | PRIME EDITOR SYSTEM FOR IN VIVO |
| | | GENOME EDITING |
| WO 2022/169235 A1 | Aug. 11, 2022 | PRIME EDITING COMPOSITION WITH |
| | | IMPROVED EDITING EFFICIENCY |
| WO 2022/150790 A3 | Aug. 11, 2022 | PRIME EDITOR VARIANTS, CONSTRUCTS, |
| | | AND METHODS FOR ENHANCING PRIME |
| | | EDITING EFFICIENCY AND PRECISION |
| WO 2022/149166 A1 | Jul. 14, 2022 | A COCKTAIL FORMULATION FOR |
| | | SELECTIVE ENRICHMENT OF GENE- |
| | | MODIFIED CELLS |
| WO 2022/150790 A2 | Jul. 14, 2022 | PRIME EDITOR VARIANTS, CONSTRUCTS, |
| | | AND METHODS FOR ENHANCING PRIME |
| | | EDITING EFFICIENCY AND PRECISION |
| U.S. Pat. No. 11,384,353 B2 | Jul. 12, 2022 | Inhibition of unintended mutations in gene |
| | | editing |
| WO 2022/067130 A3 | Jun. 23, 2022 | PRIME EDITING GUIDE RNAS, |
| | | COMPOSITIONS THEREOF, AND |
| | | METHODS OF USING THE SAME |
| WO 2022/114815 A1 | Jun. 2, 2022 | COMPOSITION FOR PRIME EDITING |
| | | COMPRISING TRANS-SPLICING ADENO- |
| | | ASSOCIATED VIRUS VECTOR |
| WO 2022/100662 A1 | May 19, 2022 | GENOMIC EDITING OF IMPROVED |
| | | EFFICIENCY AND ACCURACY |
| WO 2022/098765 A1 | May 12, 2022 | SPLIT PRIME EDITING PLATFORMS |
| WO 2022/098885 A1 | May 12, 2022 | PRECISE GENOME DELETION AND |
| | | REPLACEMENT METHOD BASED ON |
| | | PRIME EDITING |
| WO 2022/071745 A1 | Apr. 7, 2022 | PRIME EDITING USING HIV REVERSE |
| | | TRANSCRIPTASE AND CAS9 OR VARIANT |
| | | THEREOF |
| WO 2022/067130 A2 | Mar. 31, 2022 | PRIME EDITING GUIDE RNAS, |
| | | COMPOSITIONS THEREOF, AND |
| | | METHODS OF USING THE SAME |
| WO 2022/065689 A1 | Mar. 31, 2022 | PRIME EDITING-BASED GENE EDITING |
| | | COMPOSITION WITH ENHANCED EDITING |
| | | EFFICIENCY AND USE THEREOF |
| US 2022/0064626 A1 | Mar. 3, 2022 | INHIBITION OF UNINTENDED |
| | | MUTATIONS IN GENE EDITING |
| WO 2022/032085 A1 | Feb. 10, 2022 | TARGETED SEQUENCE INSERTION |
| | | COMPOSITIONS AND METHODS |
| WO 2022/025623 A1 | Feb. 3, 2022 | SYSTEM AND METHOD FOR PRIME |
| | | EDITING EFFICIENCY PREDICTION USING |
| | | DEEP LEARNING |
| WO 2021/226558 A8 | Jan. 13, 2022 | METHODS AND COMPOSITIONS FOR |
| | | SIMULTANEOUS EDITING OF BOTH |
| | | STRANDS OF A TARGET DOUBLE- |
| | | STRANDED NUCLEOTIDE SEQUENCE |
| WO 2021/243289 A1 | Dec. 2, 2021 | SYSTEMS AND METHODS FOR STABLE |
| | | AND HERITABLE ALTERATION BY |
| | | PRECISION EDITING (SHAPE) |
| WO 2021/226558 A1 | Nov. 11, 2021 | METHODS AND COMPOSITIONS FOR |
| | | SIMULTANEOUS EDITING OF BOTH |
| | | STRANDS OF A TARGET DOUBLE- |
| | | STRANDED NUCLEOTIDE SEQUENCE |
| WO 2021/215897 A1 | Oct. 28, 2021 | GENOME EDITION USING CAS9 OR CAS9 |
| | | VARIANT |
| WO 2021/215827 A1 | Oct. 28, 2021 | GENOME EDITING USING CAS9 OR CAS9 |
| | | VARIANT |
| WO 2020/191248 A8 | Oct. 21, 2021 | METHODS AND COMPOSITIONS FOR |
| | | EDITING NUCLEOTIDE SEQUENCES |
| WO 2020/191234 A8 | Oct. 21, 2021 | METHODS AND COMPOSITIONS FOR |
| | | EDITING NUCLEOTIDE SEQUENCES |
| WO 2021/165508 A1 | Aug. 26, 2021 | PRIME EDITING TECHNOLOGY FOR |
| | | PLANT GENOME ENGINEERING |
| WO 2021/138469 A1 | Jul. 8, 2021 | GENOME EDITING USING REVERSE |
| | | TRANSCRIPTASE ENABLED AND FULLY |
| | | ACTIVE CRISPR COMPLEXES |
| WO 2021/092204 A1 | May 14, 2021 | METHODS AND COMPOSITIONS FOR |
| | | NUCLEIC ACID-GUIDED NUCLEASE CELL |
| | | TARGETING SCREEN |
| WO 2021/076876 A1 | Apr. 22, 2021 | GENOTYPING EDITED MICROBIAL |
| | | STRAINS |
| WO 2021/072328 A1 | Apr. 15, 2021 | METHODS AND COMPOSITIONS FOR |
| | | PRIME EDITING RNA |
| WO 2020/191153 A8 | Dec. 30, 2020 | METHODS AND COMPOSITIONS FOR |
| | | EDITING NUCLEOTIDE SEQUENCES |
| WO 2020/191153 A3 | Dec. 10, 2020 | METHODS AND COMPOSITIONS FOR |
| | | EDITING NUCLEOTIDE SEQUENCES |
| WO 2020/191153 A9 | Nov. 12, 2020 | METHODS AND COMPOSITIONS FOR |
| | | EDITING NUCLEOTIDE SEQUENCES |
| WO 2020/191171 A9 | Oct. 29, 2020 | METHODS AND COMPOSITIONS FOR |
| | | EDITING NUCLEOTIDE SEQUENCES |
| WO 2020/191248 A1 | Sep. 24, 2020 | METHOD AND COMPOSITIONS FOR |
| | | EDITING NUCLEOTIDE SEQUENCES |
| WO 2020/191239 A1 | Sep. 24, 2020 | METHODS AND COMPOSITIONS FOR |
| | | EDITING NUCLEOTIDE SEQUENCES |
| WO 2020/191153 A2 | Sep. 24, 2020 | METHODS AND COMPOSITIONS FOR |
| | | EDITING NUCLEOTIDE SEQUENCES |
| WO 2020/191246 A1 | Sep. 24, 2020 | METHODS AND COMPOSITIONS FOR |
| | | EDITING NUCLEOTIDE SEQUENCES |
| WO 2020/191249 A1 | Sep. 24, 2020 | METHODS AND COMPOSITIONS FOR |
| | | EDITING NUCLEOTIDE SEQUENCES |
| WO 2020/191233 A1 | Sep. 24, 2020 | METHODS AND COMPOSITIONS FOR |
| | | EDITING NUCLEOTIDE SEQUENCES |
| WO 2020/191243 A1 | Sep. 24, 2020 | METHODS AND COMPOSITIONS FOR |
| | | EDITING NUCLEOTIDE SEQUENCES |
| WO 2020/191234 A1 | Sep. 24, 2020 | METHODS AND COMPOSITIONS FOR |
| | | EDITING NUCLEOTIDE SEQUENCES |
| WO 2020/191245 A1 | Sep. 24, 2020 | METHODS AND COMPOSITIONS FOR |
| | | EDITING NUCLEOTIDE SEQUENCES |
| WO 2020/191242 A1 | Sep. 24, 2020 | METHODS AND COMPOSITIONS FOR |
| | | EDITING NUCLEOTIDE SEQUENCES |
| WO 2020/191171 A1 | Sep. 24, 2020 | METHODS AND COMPOSITIONS FOR |
| | | EDITING NUCLEOTIDE SEQUENCES |
| WO 2020/191241 A1 | Sep. 24, 2020 | METHODS AND COMPOSITIONS FOR |
| | | EDITING NUCLEOTIDE SEQUENCES |
| WO 2020/156575 A1 | Aug. 6, 2020 | INHIBITION OF UNINTENDED |
| | | MUTATIONS IN GENE EDITING |
| U.S. Pat. No. 10,189,831 B2 | Jan. 29, 2019 | Non-nucleoside reverse transcriptase inhibitors |
| WO 2019/014564 A1 | Jan. 17, 2019 | SYSTEMS AND METHODS FOR TARGETED |
| | | INTEGRATION AND GENOME EDITING |
| | | AND DETECTION THEREOF USING |
| | | INTEGRATED PRIMING SITES |
| U.S. Pat. No. 10,150,955 B2 | Dec. 11, 2018 | Stabilized reverse transcriptase fusion proteins |
| WO 2018/049168 A1 | Mar. 15, 2018 | HIGH-THROUGHPUT PRECISION GENOME |
| | | EDITING |
| U.S. Pat. No. 9,783,791 B2 | Oct. 10, 2017 | Mutant reverse transcriptase and methods of use |
| U.S. Pat. No. 9,458,484 B2 | Oct. 4, 2016 | Reverse transcriptase mixtures with improved |
| | | storage stability |

In some embodiments, the gene editing system comprises a TnpB prime editing system or a polynucleotide encoding a prime editing system. In some embodiments, the cargo comprises a component of a prime editing system or a polynucleotide encoding a component of a prime editing system.

Prime editing is a versatile and precise genome editing method that directly writes new genetic information into a specified DNA site using a catalytically impaired Cas fused to an engineered reverse transcriptase, also referred to as a prime editor, which is programmable using a prime editing guide RNA (“pegRNA”) that both specifies the target site and encodes the desired edit (see, e.g., Anzalone et al., Nature 2019). Prime editing bypasses the need for DNA donor templates by using a prime editor having nickase or catalytically impaired enzymatic activity.

A prime editing system comprises a prime editor. The prime editor (“PE”) may comprise a catalytically impaired Cas protein (in the case of the present disclosure, a catalytically TnpB protein) fused to an engineered reverse transcriptase which can precisely and permanently edit one or more target nucleobases in a target polynucleotide.

In some embodiments, the prime editor comprises an engineered Moloney murine leukemia virus (“M-MLV”) reverse transcriptase (“RT”) fused to a Cas-H840A nickase (called “PE2”). In some embodiments, the prime editor comprises an engineered M-MLV RT fused to a Cas9-H840A nickase. In some embodiments, the prime editor comprises an engineered M-MLV RT fused to a TnpB of Table A. PE modifications include increased PAM flexibility to increase the utility of PE editing, expanding the coverage of targetable pathogenic variants in the Clin Var database that can now be prime edited to 94.4%.

In some embodiments, the prime editing system further comprises a prime editing guide RNA (“pegRNA”). In some embodiments, the cargo comprises a pegRNA or a polynucleotide encoding a pegRNA. In the case of TnpB, a TnpB guide RNA can be modified to include an equivalent “extension arm” at 3′ or 5′ of the reRNA to provide a primer binding site (PBS) for binding to the 3′ end to the nicked strand and which initiates reverse transcription, and the RT template, which encodes a sequence that includes a desired edit and which becomes integrated in place of the endogenous strand downstream of the nick site.

In some embodiments, the prime editing system further comprises a second guide RNA targeting the complementary strand, allowing the Cas9 nickase to also nick the non-edited strand (called “PE3”), which biases mismatch DNA repair in favor of the edited sequence. In some embodiments, the second guide RNA is designed to recognize the complementary strand of DNA only after the PE3 edit has occurred (called “PE3b”), which reduces indel formation.

In some embodiments, the prime editing system comprises an uracil glycosylase inhibitor. In some embodiments, the prime editing system comprises a Cas9 protein fused to an uracil glycosylase inhibitor. In some embodiments, the cargo comprises an uracil glycosylase inhibitor or a polynucleotide encoding an uracil glycosylase inhibitor. In some embodiments, the cargo comprises a Cas9 protein fused to an uracil glycosylase inhibitor or a polynucleotide encoding a Cas9 protein fused to an uracil glycosylase inhibitor.

Any of the above prime editor embodiments or variants, modifications, or derivatives thereof are contemplated herein to be delivered by the LNP systems disclosed in this specification for gene editing in cells, tissues, and/or organs under in vitro, ex vivo, or in vivo conditions. The various components described herein may be configured and delivered in any suitable manner. Any of the descriptions presented in this section are not intended to be strictly limiting.

In additional embodiments, the TnpB-deaminase fusion protein is co-expressed with a TnpB not fused to a reverse transcriptase. Preferably, the unfused TnpB is mutated to be catalytically inactive, however, fused TnpB may also be mutated to be catalytically inactive, either or both. Various TnpB-RT fusion protein binds to a truncated reRNA or to a truncated guide RNA. In some embodiments, this maintains DNA binding activity but slows cleavage kinetics or deactivates DNA cleavage partially or entirely. Additional embodiments, include the reverse transcriptase fused to the N-terminus of TnpB or to the C-terminus of TnpB. In further embodiments, the reverse transcriptase is placed inside the Rec-domain of the TnpB, after the Rec-domain of the TnpB, in the Wedge domain of TnpB, after the Wedge domain of TnpB, in the RuvC domain of TnpB, after the RuvC domain of TnpB, in the Helical hairpin domain of TnpBafter the Helical hairpin domain of TnpB, in the ZnF domain of TnpB, after the Znf domain of TnpB.

Preferably, the TnpB-RT fusion protein is bound to an engineered reRNA wherein the engineered reRNA contains a 5′ extension, the engineered reRNA contains a 3′ extension, the extensions contain a template for a desired edit, the extension contains homology to the target site, the extension contains homology to the human genome, the extension contains sequence encoding a landing-pad for a homing integrase and/or recombinase. In preferred embodiments, the TnpB-RT fusion protein is fused or cleaved. In certain embodiments, the TnpB-RT system is fused to a polypeptide that modulates host-repair, wherein the polypeptide is a uracil glycosylase inhibitor, wherein the polypeptide inhibits mismatch repair, wherein the MMR inhibiting polypeptide is a dominant negative MLH1.

### TnpB Transcription Modulating Systems

In various aspects of the invention, the TnpB may be fused to a transcriptional modulating polypeptide suitable for transcriptional interference, activation or epigenetic editing.

In some embodiments, the TnpB-transcriptional modulating polypeptide fusions comprise one or more nuclear localization signals selected or derived from SV40, c-Myc or NLP-1.

In other embodiments, the TnpB-transcriptional modulating polypeptide fusion proteins bind to a truncated guide RNA. In further embodiments, the TnpB-transcriptional modulating polypeptide comprises glycine and serine residues. In yet other embodiments, the TnpB-transcriptional modulating polypeptide are linked to one or more unstructured XTEN protein polymers.

In various embodiments, the transcriptional modulating polypeptide of the TnpB-transcriptional modulating polypeptide fusion performs histone acetylation or comprises histone acetyltransferase (HAT) p300 activity.

In other embodiments, the transcriptional modulating polypeptide of the TnpB-transcriptional modulating polypeptide fusion performs histone demethylation or comprises lysine-specific demethylase (LSD1) activity.

In further embodiments, the transcriptional modulating polypeptide of the TnpB-transcriptional modulating polypeptide fusion performs cystine methylation or comprises one or more activities selected from DNA (cytosine-5)-methyltransferase (DNMT3A), DNA-methyltransferase 3-like (DNMT3L) and MQ1.

In other embodiments, the transcriptional modulating polypeptide of the TnpB-transcriptional modulating polypeptide fusion performs cystine demethylation or comprises TET1 activity.

In additional embodiments, the transcriptional modulating peptide of the TnpB-transcriptional modulating polypeptide fusion is a transcriptional repressor or comprises a KRAB domain. Alternatively, the transcriptional modulating peptide of the TnpB-transcriptional modulating polypeptide fusion is a transcriptional activator or comprises one or more activators including without limitation, for example, HS1, VP64 and p65.

In other embodiments, where the transcriptional modulating peptide of the TnpB-transcriptional modulating polypeptide fusion is a repressor or comprises multiple transcriptional modulating peptides. In yet other embodiments, the TnpB of the TnpB-transcriptional modulating polypeptide fusion is mutated to be catalytically inactive.

In further embodiments, the transcriptional modulating peptides of the TnpB-transcriptional modulating polypeptide fusion are physically coupled through an engineered reRNA wherein the reRNA comprises one or more aptamers.

In additional embodiments, the transcriptional modulating peptides of the TnpB-transcriptional modulating polypeptide fusion are physically coupled through an engineered guide RNA, wherein the guide RNA contains one or more aptamers.

### Other TnpB Modifications

### Nuclear Localization Sequences

In one embodiment, the TnpB polypeptide is fused to one or more nuclear localization sequences (NLSs), such as about or more than about 1, 2, 3, 4, 5, 6, 7, 8, 9, 10, or more NLSs. In one embodiment, the TnpB polypeptide comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In an embodiment of the invention, the TnpB polypeptide comprises at most 6 NLSs. In one embodiment, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Nonlimiting examples of NLSs include an NLS sequence derived from: the NLS of the SV40 virus large T-antigen, having the amino acid sequence PKKKRKV (SEQ ID NO: 302); the NLS from nucleoplasmin (e.g. the nucleoplasmin bipartite NLS with the sequence KRPAATKKAGQAKKKK (SEQ ID NO: 303); the c-myc NLS having the amino acid sequence PAAKRVKLD (SEQ ID NO: 304) or RQRRNELKRSP (SEQ ID NO: 305); the hRNPAI M9 NLS having the sequence NQSSNFGPMKGGNFGGRSSGPYGGGGQYFAKPRNQGGY (SEQ ID NO: 306); the sequence RMRIZFKNKGKDTAELRRRRVEVSVELRKAKKDEQILKRRNV (SEQ ID NO: 307) of the IBB domain from importin-alpha; the sequences VSRKRPRP (SEQ ID NO: 308) and PPKKARED (SEQ ID NO: 309) of the myoma T protein; the sequence PQPKKKPL (SEQ ID NO: 310) of human p53; the sequence SALIKKKKKMAP (SEQ ID NO: 311) of mouse c-abl IV; the sequences DRLRR (SEQ ID NO: 312) and PKQKKRK (SEQ ID NO: 313) of the influenza virus NS 1; the sequence RKLKKKIKKL (SEQ ID NO: 314) of the Hepatitis virus delta antigen; the sequence REKKKFLKRR (SEQ ID NO: 315) of the mouse Mxl protein; the sequence KRKGDEVDGVDEVAKKKSKK (SEQ ID NO: 316) of the human poly (ADP-ribose) polymerase; and the sequence RI<CLQAGMNLEARI<TI<I< (SEQ ID NO: 317) of the steroid hormone receptors (human) glucocorticoid.

In general, the one or more NLSs are of sufficient strength to drive accumulation of the TnpB polypeptide (or an NLS-modified accessory protein, or an NLS-modified chimera comprising a TnpB protein and an accessory protein) in a detectable amount in the nucleus of a eukaryotic cell. In general, strength of nuclear localization activity may derive from the number of NLSs in the TnpB polypeptide, the particular NLS(s) used, or a combination of these factors. Detection of accumulation in the nucleus may be performed by any suitable technique.

For example, a detectable marker may be fused to the TnpB polypeptide, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of complex formation (e.g., assay for DNA cleavage or mutation at the target sequence, or assay for altered gene expression activity affected by complex formation and/or TnpB polypeptide activity), as compared to a control no exposed to the TnpB polypeptide or complex, or exposed to a TnpB polypeptide lacking the one or more NLSs. In one embodiment of the herein described TnpB polypeptide protein complexes and systems the codon optimized TnpB polypeptide proteins comprise an NLS attached to the C-terminal of the protein. In one embodiment, other localization tags may be fused to the TnpB polypeptide, such as without limitation for localizing the TnpB polypeptide to particular sites in a cell, such as organelles, such as mitochondria, plastids, chloroplast, vesicles, golgi, (nuclear or cellular) membranes, ribosomes, nucleolus, ER, cytoskeleton, vacuoles, centrosome, nucleosome, granules, centrioles, etc.

In one embodiment of the invention, at least one nuclear localization signal (NLS) is attached to the nucleic acid sequences encoding the TnpB polypeptide. In preferred embodiments at least one or more C-terminal or N-terminal NLSs are attached (and hence nucleic acid molecule(s) coding for the TnpB polypeptide can include coding for NLS(s) so that the expressed product has the NLS(s) attached or connected). In a preferred embodiment a C-terminal NLS is attached for optimal expression and nuclear targeting in eukaryotic cells, preferably human cells. The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

In other examples, the fusion proteins comprising TnpB and another accessory protein (e.g., RT) contains one or more nuclear localization signals is selected or derived from SV40, c-Myc or NLP-1.

The NLS examples above are non-limiting. The TnpB fusion proteins contemplated herein may comprise any known NLS sequence, including any of those described in Cokol et al., “Finding nuclear localization signals,” EMBO Rep., 2000, 1 (5): 411-415 and Freitas et al., “Mechanisms and Signals for the Nuclear Import of Proteins,” Current Genomics, 2009, 10 (8): 550-7, each of which are incorporated herein by reference. Linkers

In some embodiments, the TnpB polypeptides are coupled to one or more accessory functions by a linker. One or more coRNAs directed to such promoters or enhancers may also be provided to direct the binding of the TnpB polypeptide to such promoters or enhancers. The term linker as used in reference to a fusion protein refers to a molecule which joins the proteins to form a fusion protein. Generally, such molecules have no specific biological activity other than to join or to preserve some minimum distance or other spatial relationship between the proteins. However, in one embodiment, the linker may be selected to influence some property of the linker and/or the fusion protein such as the folding, net charge, or hydrophobicity of the linker.

Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the TnpB polypeptide and an accessory protein (e.g., a nucleotide deaminase) by a distance sufficient to ensure that each protein retains its required functional property. Preferred peptide linker sequences adopt a flexible extended conformation and do not exhibit a propensity for developing an ordered secondary structure. In one embodiment, the linker can be a chemical moiety which can be monomeric, dimeric, multimeric or polymeric. Preferably, the linker comprises amino acids. Typical amino acids in flexible linkers include Gly, Asn and Ser.

Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40:39-46; Murphy et al. (1986) Proc. Nat'l. Acad. Sci. USA 83:8258-62; U.S. Pat. Nos. 4,935,233; and 4,751,180. For example, GlySer linkers may be based on repeating units of GGS, i.e., up to 2, 3, 4, 5, 6, 7, 8, 9, 10, 11 or even 12 or more repeating units, including but not limited to:

| SEQ | | |
|---|---|---|
| ID | Description | Sequence |
| 318 | GlySer linker | GGS |
| | based on GGS | |
| | repeating unit | |
| 319 | GlySer linker | GGS GGS |
| | based on GGS | |
| | repeating unit | |
| 320 | GlySer linker | GGS GGS GGS |
| | based on GGS | |
| | repeating unit | |
| 321 | GlySer linker | GGS GGS GGS GGS |
| | based on GGS | |
| | repeating unit | |
| 322 | GlySer linker | GGS GGS GGS GGS GGS |
| | based on GGS | |
| | repeating unit | |
| 323 | GlySer linker | GGS GGS GGS GGS GGS GGS |
| | based on GGS | |
| | repeating unit | |
| 324 | GlySer linker | GGS GGS GGS GGS GGS GGS GGS |
| | based on GGS | |
| | repeating unit | |
| 325 | GlySer linker | GGS GGS GGS GGS GGS GGS GGS GGS |
| | based on GGS | |
| | repeating unit | |
| 326 | GlySer linker | GGS GGS GGS GGS GGS GGS GGS GGS GGS |
| | based on GGS | |
| | repeating unit | |
| 327 | GlySer linker | GGS GGS GGS GGS GGS GGS GGS GGS GGS GGS |
| | based on GGS | |
| | repeating unit | |
| 328 | GlySer linker | GGS GGS GGS GGS GGS GGS GGS GGS GGS GGS GGS |
| | based on GGS | |
| | repeating unit | |
| 329 | GlySer linker | GGS GGS GGS GGS GGS GGS GGS GGS GGS GGS GGS |
| | based on GGS | GGS |
| | repeating unit | |
| 330 | GlySer linker | GGS GGS GGS GGS GGS GGS GGS GGS GGS GGS GGS |
| | based on GGS | GGS GGS |
| | repeating unit | |
| 331 | GlySer linker | GGS GGS GGS GGS GGS GGS GGS GGS GGS GGS GGS |
| | based on GGS | GGS GGS GGS |
| | repeating unit | |
| 332 | GlySer linker | GGS GGS GGS GGS GGS GGS GGS GGS GGS GGS GGS |
| | based on GGS | GGS GGS GGS GGS |
| | repeating unit | |

In another example, GlySer linkers may be based on repeating units of GSG, i.e., up to 2, 3, 4, 5, 6, 7, 8, 9, 10, 11 or even 12 or more repeating units, including but not limited to:

| SEQ | | |
|---|---|---|
| ID | Description | Sequence |
| 333 | GlySer linker | GSG |
| | based on GSG | |
| | repeating unit | |
| 334 | GlySer linker | GSG GSG |
| | based on GSG | |
| | repeating unit | |
| 335 | GlySer linker | GSG GSG GSG |
| | based on GSG | |
| | repeating unit | |
| 336 | GlySer linker | GSG GSG GSG GSG |
| | based on GSG | |
| | repeating unit | |
| 337 | GlySer linker | GSG GSG GSG GSG GSG |
| | based on GSG | |
| | repeating unit | |
| 338 | GlySer linker | GSG GSG GSG GSG GSG GSG |
| | based on GSG | |
| | repeating unit | |
| 339 | GlySer linker | GSG GSG GSG GSG GSG GSG GSG GSG |
| | based on GSG | |
| | repeating unit | |
| 340 | GlySer linker | GSG GSG GSG GSG GSG GSG GSG GSG GSG |
| | based on GSG | |
| | repeating unit | |
| 341 | GlySer linker | GSG GSG GSG GSG GSG GSG GSG GSG GSG GSG |
| | based on GSG | |
| | repeating unit | |
| 342 | GlySer linker | GSG GSG GSG GSG GSG GSG GSG GSG GSG GSG GSG |
| | based on GSG | |
| | repeating unit | |
| 343 | GlySer linker | GSG GSG GSG GSG GSG GSG GSG GSG GSG GSG GSG |
| | based on GSG | GSG |
| | repeating unit | |
| 344 | GlySer linker | GSG GSG GSG GSG GSG GSG GSG GSG GSG GSG GSG |
| | based on GSG | GSG GSG |
| | repeating unit | |
| 345 | GlySer linker | GSG GSG GSG GSG GSG GSG GSG GSG GSG GSG GSG |
| | based on GSG | GSG GSG GSG |
| | repeating unit | |
| 346 | GlySer linker | GSG GSG GSG GSG GSG GSG GSG GSG GSG GSG GSG |
| | based on GSG | GSG GSG GSG GSG |
| | repeating unit | |

In yet another example, GlySer linkers may be based on repeating units of GGGS, i.e., up to 2, 3, 4, 5, 6, 7, 8, 9, 10, 11 or even 12 or more repeating units, including but not limited to:

| SEQ | | |
|---|---|---|
| ID | Description | Sequence |
| 347 | GlySer linker | GGGS |
| | based on | |
| | GGGS | |
| | repeating unit | |
| 348 | GlySer linker | GGGS GGGS |
| | based on | |
| | GGGS | |
| | repeating unit | |
| 349 | GlySer linker | GGGS GGGS GGGS |
| | based on | |
| | GGGS | |
| | repeating unit | |
| 350 | GlySer linker | GGGS GGGS GGGS GGGS |
| | based on | |
| | GGGS | |
| | repeating unit | |
| 351 | GlySer linker | GGGS GGGS GGGS GGGS GGGS |
| | based on | |
| | GGGS | |
| | repeating unit | |
| 352 | GlySer linker | GGGS GGGS GGGS GGGS GGGS GGGS |
| | based on | |
| | GGGS | |
| | repeating unit | |
| 353 | GlySer linker | GGGS GGGS GGGS GGGS GGGS GGGS GGGS |
| | based on | |
| | GGGS | |
| | repeating unit | |
| 354 | GlySer linker | GGGS GGGS GGGS GGGS GGGS GGGS GGGS GGGS |
| | based on | |
| | GGGS | |
| | repeating unit | |
| 355 | GlySer linker | GGGS GGGS GGGS GGGS GGGS GGGS GGGS GGGS GGGS |
| | based on | |
| | GGGS | |
| | repeating unit | |
| 356 | GlySer linker | GGGS GGGS GGGS GGGS GGGS GGGS GGGS GGGS GGGS |
| | based on | GGGS |
| | GGGS | |
| | repeating unit | |
| 357 | GlySer linker | GGGS GGGS GGGS GGGS GGGS GGGS GGGS GGGS GGGS |
| | based on | GGGS GGGS |
| | GGGS | |
| | repeating unit | |
| 358 | GlySer linker | GGGS GGGS GGGS GGGS GGGS GGGS GGGS GGGS GGGS |
| | based on | GGGS GGGS GGGS |
| | GGGS | |
| | repeating unit | |
| 359 | GlySer linker | GGGS GGGS GGGS GGGS GGGS GGGS GGGS GGGS GGGS |
| | based on | GGGS GGGS GGGS GGGS |
| | GGGS | |
| | repeating unit | |
| 360 | GlySer linker | GGGS GGGS GGGS GGGS GGGS GGGS GGGS GGGS GGGS |
| | based on | GGGS GGGS GGGS GGGS GGGS |
| | GGGS | |
| | repeating unit | |
| 361 | GlySer linker | GGGS GGGS GGGS GGGS GGGS GGGS GGGS GGGS GGGS |
| | based on | GGGS GGGS GGGS GGGS GGGS GGGS |
| | GGGS | |
| | repeating unit | |

In still another example, GlySer linkers may be based on repeating units of GGGGS, i.e., up to 2, 3, 4, 5, 6, 7, 8, 9, 10, 11 or even 12 or more repeating units, including but not limited to:

| SEQ | | |
|---|---|---|
| ID | Description | Sequence |
| 362 | GlySer linker | GGGGS |
| | based on | |
| | GGGGS | |
| | repeating unit | |
| 363 | GlySer linker | GGGGS GGGGS |
| | based on | |
| | GGGGS | |
| | repeating unit | |
| 364 | GlySer linker | GGGGS GGGGS GGGGS |
| | based on | |
| | GGGGS | |
| | repeating unit | |
| 365 | GlySer linker | GGGGS GGGGS GGGGS GGGGS |
| | based on | |
| | GGGGS | |
| | repeating unit | |
| 366 | GlySer linker | GGGGS GGGGS GGGGS GGGGS GGGGS |
| | based on | |
| | GGGGS | |
| | repeating unit | |
| 367 | GlySer linker | GGGGS GGGGS GGGGS GGGGS GGGGS GGGGS |
| | based on | |
| | GGGGS | |
| | repeating unit | |
| 368 | GlySer linker | GGGGS GGGGS GGGGS GGGGS GGGGS GGGGS GGGGS |
| | based on | |
| | GGGGS | |
| | repeating unit | |
| 369 | GlySer linker | GGGGS GGGGS GGGGS GGGGS GGGGS GGGGS GGGGS |
| | based on | GGGGS |
| | GGGGS | |
| | repeating unit | |
| 370 | GlySer linker | GGGGS GGGGS GGGGS GGGGS GGGGS GGGGS GGGGS |
| | based on | GGGGS GGGGS |
| | GGGGS | |
| | repeating unit | |
| 371 | GlySer linker | GGGGS GGGGS GGGGS GGGGS GGGGS GGGGS GGGGS |
| | based on | GGGGS GGGGS GGGGS |
| | GGGGS | |
| | repeating unit | |
| 372 | GlySer linker | GGGGS GGGGS GGGGS GGGGS GGGGS GGGGS GGGGS |
| | based on | GGGGS GGGGS GGGGS GGGGS |
| | GGGGS | |
| | repeating unit | |
| 373 | GlySer linker | GGGGS GGGGS GGGGS GGGGS GGGGS GGGGS GGGGS |
| | based on | GGGGS GGGGS GGGGS GGGGS GGGGS |
| | GGGGS | |
| | repeating unit | |
| 374 | GlySer linker | GGGGS GGGGS GGGGS GGGGS GGGGS GGGGS GGGGS |
| | based on | GGGGS GGGGS GGGGS GGGGS GGGGS GGGGS |
| | GGGGS | |
| | repeating unit | |
| 375 | GlySer linker | GGGGS GGGGS GGGGS GGGGS GGGGS GGGGS GGGGS |
| | based on | GGGGS GGGGS GGGGS GGGGS GGGGS GGGGS GGGGS |
| | GGGGS | |
| | repeating unit | |
| 376 | GlySer linker | GGGGS GGGGS GGGGS GGGGS GGGGS GGGGS GGGGS |
| | based on | GGGGS GGGGS GGGGS GGGGS GGGGS GGGGS GGGGS |
| | GGGGS | GGGGS |
| | repeating unit | |

In yet a further embodiment, LEPGEKPYKCPECGKSFSQSGALTRHQRTHTR (SEQ ID NO: 377) is used as a linker.

In yet an additional embodiment, the linker is an XTEN linker, which is TCGGGATCTGAGACGCCTGGGACCTCGGAATCGGCTACGCCCGAAAGT (SEQ ID NO. 378). In particular embodiments, the TnpB polypeptide is linked to the deaminase protein or its catalytic domain by means of an LEPGEKPYKCPECGKSFSQSGALTRHQRTHTR LEPGEKPYKCPECGKSFSQSGALTRHQRTHTR (SEQ ID NO: 379) linker. In further particular embodiments, TnpB polypeptide is linked C-terminally to the N-terminus of a deaminase protein or its catalytic domain by means of an LEPGEKPYKCPECGKSFSQSGALTRHQRTHTRLEPGEKPYKCPECGKSFSQSGALTRH QRTHTRLEPGEKPYKCPECGKSFSQSGALTRHQRTHTR (SEQ ID NO: 380) linker. In addition, N- and C-terminal NLSs can also function as linker (e.g., PKKKRKVEASSPKKRKVEAS (SEQ ID NO: 381)).

The above description of linkers is intended to be non-limiting and includes any combinations of the above linkers or heterologous combinations of repeating GlySer linkers.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminoHEXAnoic acid (Ahx). In certain embodiments, the linker is based on a carbocyclic moiety (e.g., cyclopentane, cycloHEXAne). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included funtionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

### Inteins

It will be understood that in some embodiments (e.g., delivery of a TnpB proteins and editing systems in vivo using AAV particles), it may be advantageous to split a polypeptide (e.g., a TnpB protein or a fusion protein comprising TnpB) into an N-terminal half and a C-terminal half, delivery them separately, and then allow their colocalization to reform the complete protein (or fusion protein as the case may be) within the cell. Separate halves of a protein or a fusion protein may each comprise a split-intein tag to facilitate the reformation of the complete protein or fusion protein by the mechanism of protein trans splicing.

Protein trans-splicing, catalyzed by split inteins, provides an entirely enzymatic method for protein ligation. A split-intein is essentially a contiguous intein (e.g. a mini-intein) split into two pieces named N-intein and C-intein, respectively. The N-intein and C-intein of a split intein can associate non-covalently to form an active intein and catalyze the splicing reaction essentially in same way as a contiguous intein does. Split inteins have been found in nature and also engineered in laboratories. As used herein, the term “split intein” refers to any intein in which one or more peptide bond breaks exists between the N-terminal and C-terminal amino acid sequences such that the N-terminal and C-terminal sequences become separate molecules that can non-covalently reassociate, or reconstitute, into an intein that is functional for trans-splicing reactions. Any catalytically active intein, or fragment thereof, may be used to derive a split intein for use in the methods of the invention. For example, in one aspect the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing trans-splicing reactions.

### Rerna (Tnpb Ncrna)

The TnpB systems herein may further comprise one or more nucleic acid components, which are also referred to herein as reRNA or TnpB ncRNAs. As reported in Karvelis et al., “Transposon-associated TnpB is a programmable RNA-guided DNA endonuclease,” Nature, Nov. 25, 2021, Vol. 599, pp. 692-700 (incorporated herein by reference), TnpB is an RNA-guided dsDNA nuclease that forms a complex with a non-coding RNA called “reRNA.” The reRNA is a transcript that is generated from the transcription of the IS DNA sequence beginning at a transcription initiation site located within 3′ end of the TnpB coding region and ending at a transcription termination site located in the flanking genomic DNA region that is immediately downstream of the RE of the Insertion Sequence. See FIG. 1. Thus, the reRNA comprises three regions: (a) a region corresponding to 3′ end of the TnpB coding region, (b) a region corresponding to the RE, and (c) a region corresponding to the flanking genomic DNA immediately downstream of 3′ end of the RE. Regions (a) and (b) generally form a folded “scaffold” that appears to bind to the TnpB protein and may be regarded as a single region (as depicted in FIG. 1). Region (c) functions as a spacer/guide or targeting sequence which allows for the targeting of a TnpB-reRNA complex to a target site to which the region (c) has complementarity to and anneals. Region (c), in various embodiments, can be engineered to be any desired target sequence such that the TnpB-reRNA complex is targeted to a desired target sequence.

Thus, the reRNA sequence may be predicted from the sequence of the region spanning 3′ end of the TnpB coding region through a flanking region downstream of the RE. Example 9 describes a method for predicting the reRNA sequence as spanning a region from the last functional domain (e.g., ZF domain) in TnpB through a position in the downstream adjacent flanking DNA that marks the beginning of the loss of conserved sequence alignment among loci comprising TnpA-TnpB IS operons with flanking regions (see FIG. 1). Exemplary predicted reRNA are shown in Table B. In certain embodiments, the TnpB editing system comprises a TnpB and a predicted reRNA of the same TnpB accession number. In certain embodiments, the TnpB editing system comprises a TnpB and a predicted reRNA from different TnpB accession numbers. That is, one may use any particular TnpB protein from Table A with its cognate reRNA in Table B. However, one may also combine a TnpB protein from Table A with any reRNA from Table B which is not sourced from the same TnpB accession number. The predicted reRNA of Table B are referred to as “reRNA containing regions” which can be further processed/shortened in accordance with known methods described herein and in the literature, for example, in Meers et al., “Transposon-encoded nuclease use guide RNAs to selfishly bias their inheritance,” BioRxiv, Mar. 14, 2023 and Sasnauskas et al., “TnpB structure reveals minimal functional core of Cas12 nuclease family,” Nature, Vol. 616, Apr. 13, 2023, each of which are incorporated herein by reference.

Computational methods were used to predict the reRNA sequences for the identified TnpB-like proteins of Table B. As reported in Karvelis et al., “Transposon-associated TnpB is a programmable RNA-guided DNA endonuclease,” Nature, Nov. 25, 2021, Vol. 599, pp. 692-700, the TnpB protein co-purified with an RNA molecule of about 150 nucleotides long which had a sequence that was derived from the IS and a sequence downstream of the IS.

In various embodiments, reRNA may be engineered to include RNA, DNA, or combinations of both and include modified and non-canonical nucleotides as described further below. The reRNA can comprise a reprogrammable spacer sequence and a scaffold that interacts with the TnpB polypeptide. reRNA may form a complex with a TnpB polypeptide, and direct sequence-specific binding of the complex to a target sequence of a target polynucleotide. In one example embodiment, the reRNA is a single molecule comprising a scaffold sequence and a spacer sequence. In certain example embodiments, the spacer is 5′ of the scaffold sequence. In one example embodiment, the reRNA may further comprise a conserved nucleic acid sequence between the scaffold and spacer portions.

In embodiments, the reRNA comprises a spacer sequence and a scaffold sequence, e.g. a conserved nucleotide sequence. In embodiments, the reRNA comprises about 45 to about 350 nucleotides, or about 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 17, 138, 19, 140, 141, 142, 143, 144, 145, 146, 147, 148, 149, 150, 151, 152, 153, 154, 155, 156, 157, 158, 159, 160, 11, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 2340, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 272, 273, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 333, 334, 335, 336, 337, 338, 339, 340, 341, 342, 343, 344, 345, 346, 347, 348, 349, or 350 nucleotides.

In embodiments, the reRNA comprises a scaffold sequence, e.g. a conserved nucleotide sequence that binds to the TnpB protein. The scaffold sequence therefore typically comprises conserved regions, with the scaffold comprising about 30 to 200 nucleotides, about 50 to 180, about 80 to 175 nucleotides, or about 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180 or more nucleotides.

The reRNA may further comprise a spacer, which can be re-programmed to direct site specific binding to a target sequence of a target polynucleotide. The spacer may also be referred to herein as part of the reRNA scaffold or reRNA, and may comprise an engineered heterologous sequence.

In one embodiment, the spacer length or targeting sequence length of the reRNA is from 10 to 50 nt. In one embodiment, the spacer length of the oRNA is at least 10, 11, 12, 13, 14, or 15 nucleotides. In one embodiment, the spacer length is from 10 to 40 nuecleotides, from 15 to 30 nt, 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer. In example embodiments, the spacer sequence is 14, 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, or 50 nt.

As used herein, the term “spacer” may also be referred to as a “guide sequence” or “targeting sequence” which has complementarity to a target sequence (e.g., a desired target gene in a genome which is desired to be edited). In one embodiment, the degree of complementarity of the spacer sequence to a given target sequence, when optimally aligned using a suitable alignment algorithm, is about or more than 50%, 60%, 75%, 80%, 85%, 90%, 95%, 97.5%, 99%, or more. In certain example embodiments, the reRNA molecule comprises a spacer sequence that may be designed to have at least one mismatch with the target sequence, such that a RNA duplex formed between the sequence and the target sequence. Accordingly, the degree of complementarity is less than 99%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies), ELAND (Illumina, San Diego, CA), SOAP (for example, as described by Li, et al. Bioinformatics. 24 (5): 713-714; and Liu, et al. Bioinformatics 28 (6): 878-879.), and Maq (for example, as described by Li, et al. Genome Res. 2008 November;18 (11): 1851-8.).

The ability of a sequence (within a nucleic acid-targeting reRNA molecule) to direct sequence-specific binding of a nucleic acid-targeting complex to a target nucleic acid sequence may be assessed by any suitable assay. For example, the components of a reRNA system sufficient to form a TnpB-targeting complex, including the reRNA molecule sequence to be tested, may be provided to a host cell having the corresponding target nucleic acid sequence, such as by transfection with vectors encoding the components of the TnpB-targeting complex, followed by an assessment of preferential targeting (e.g., cleavage) within the target nucleic acid sequence. Similarly, cleavage of a target nucleic acid sequence (or a sequence in the vicinity thereof) may be evaluated in a test tube by providing the target nucleic acid sequence, components of a TnpB-targeting complex, including the sequence to be tested and a control sequence different from the test coRNA, and comparing binding or rate of cleavage at or in the vicinity of the target sequence between the test and control reRNA molecule sequence reactions. Other assays are possible, and will occur to those skilled in the art. A spacer sequence, and hence a nucleic acid targeting reRNA may be selected to target any target nucleic acid sequence.

reRNA Modifications

In one embodiment, the reRNA comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the reRNA sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a reRNA component nucleic acid comprises ribonucleotides and non-ribonucleotides. In one such embodiment, a reRNA component comprises one or more ribonucleotides and one or more deoxyribonucleotides. In an embodiment of the invention, the reRNA component comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA).

Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of coRNA chemical modifications include, without limitation, incorporation of 2′-O-methyl (M), 2′-O-methyl 3′phosphorothioate (MS), S-constrained ethyl (cEt), or 2′-O-methyl 3 ‘thioPACE (MSP) at one or more terminal nucleotides. Such chemically modified ORNA components can comprise increased stability and increased activity as compared to unmodified oRNA components, though on-target vs. off-target specificity is not predictable. (See, Hendel, 2015, Nat Biotechnol. 33 (9): 985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33 (9): 985-989; Li et al., Nature Biomedical Engineering, 2017, 1, 0066 D01: 10.1038/s41551-017-0066). In one embodiment, the 5′ and/or 3′ end of a reRNA component is modified by a variety of functional moieties including fluorescent dyes, polyethylene glycol, cholesterol, proteins, or detection tags. (See Kelly et al., 2016, J. Biotech. 233:74-83). In one embodiment, a reRNA component comprises ribonucleotides in a region that binds to a target sequence and one or more deoxyribonucletides and/or nucleotide analogs in a region that binds to the TnpB polypeptide.

In an embodiment, deoxyribonucleotides and/or nucleotide analogs are incorporated in engineered reRNA component structures. In one embodiment, 3-5 nucleotides at either 3′ or 5′ end of a reRNA component is chemically modified. In one embodiment, only minor modifications are introduced in the seed region, such as 2′-F modifications. In one embodiment, 2′-F modification is introduced at the 3′ end of a reRNA component. In one embodiment, three to five nucleotides at 5′ and/or 3′ end of the reRNA component are chemically modified with 2′-O-methyl (M), 2′-O-methyl 3′ phosphorothioate (MS), S-constrained ethyl (cEt), or 2′-O-methyl 3′ thioPACE (MSP). Such modification can enhance genome editing efficiency (see Hendel et al., Nat. Biotechnol. (2015) 33 (9): 985-989). In one embodiment, all of the phosphodiester bonds of a reRNA component are substituted with phosphorothioates (PS) for enhancing levels of gene disruption. In one embodiment, more than five nucleotides at 5′ and/or 3′ end of the reRNA component are chemically modified with 2′-O-Me, 2′-F or S-constrained ethyl (cEt). Such chemically modified reRNA component can mediate enhanced levels of gene disruption (see Ragdarm et al., 0215, PNAS, E7110-E7111). In an embodiment of the invention, a reRNA component is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the reRNA component by a linker, such as an alkyl chain. In one embodiment, the chemical moiety of the modified nucleic acid component can be used to attach the reRNA component to another molecule, such as DNA, RNA, protein, or nanoparticles. Such chemically modified reRNA component can be used to identify or enrich cells generically edited by a TnpB polypeptide and related systems (see Lee et al., eLife, 2017, 6: e25312, DOI: 10.7554).

Other reRNA modifications are described in Kim, D. Y., Lee, J. M., Moon, S. B. et al. Efficient CRISPR editing with a hypercompact Cas12fl and engineered guide RNAs delivered by adeno-associated virus. Nat Biotechnol 40, 94-102 (2022).

Accordingly, in various aspects of the invention, the reRNA are modified in one or more TnpB reRNA. MS1, an internal penta (uridinylate)(UUUUU) sequence in the tracrRNA; MS2, the 3′ terminus of the crRNA; MS3, the ‘stem 1’ region of the tracrRNA; MS4, the tracrRNA-crRNA complementary region; and MS5, the ‘stem 2’ region of the tracrRNA.

Various aspects of the invention provide methods and compositions for improved reRNA stability via chemical modifications. Braasch, D. A., Jensen, S., Liu, Y., Kaur, K., Arar, K., White, M. A., et al. (2003). RNA interference in mammalian cells by chemically-modified RNA. Biochemistry 42, 7967-7975. doi: 10.1021/bi0343774. Chiu, Y. L., and Rana, T. M. (2003). siRNA function in RNAi: a chemical modification analysis. RNA 9, 1034-1048. doi: 10.1261/rna.5103703. Behlke, M. A. (2008). Chemical modification of siRNAs for in vivo use. Oligonucleotides18, 305-319. doi: 10.1089/oli.2008.0164. Bennett, C. F., and Swayze, E. E. (2010). RNA targeting therapeutics: molecular mechanisms of antisense oligonucleotides as a therapeutic platform. Annu. Rev. Pharmacol. Toxicol. 50, 259-293. doi: 10.1146/annurev.pharmtox.010909.105654. Deleavey, G. F., and Damha, M. J. (2012). Designing chemically modified oligonucleotides for targeted gene silencing. Chem. Biol. 19, 937-954. doi: 10.1016/j.chembiol.2012.07.011. Lennox, K. A., and Behlke, M. A. (2020). Chemical modifications in RNA interference and CRISPR/Cas genome editing reagents. Methods Mol. Biol. 2115, 23-55. doi: 10.1007/978-1-0716-0290-4_2.

For instance, Hendel et al. improved guideRNA stability by chemically modifying gRNA ends to reduce degradation by exonucleases, RNA nuclease. Hendel, A., Bak, R. O., Clark, J. T., Kennedy, A. B., Ryan, D. E., Roy, S., et al. (2015a). Chemically modified guide RNAs enhance CRISPR-Cas genome editing in human primary cells. Nat. Biotechnol. 33, 985-989. doi: 10.1038/nbt.3290. Chemical modifications of gRNAs may enable more efficient and safer gene-editing in primary cells suitable for clinical applications.

A review of types of chemical modifications are provided in Table AA below. Allen, Daniel et al. “Using Synthetically Engineered Guide RNAs to Enhance CRISPR Genome Editing Systems in Mammalian Cells.” Frontiers in genome editing vol. 2 617910. 28 Jan. 2021, doi: 10.3389/fgeed.2020.617910

TABLE AA

| | | Effect on | |
|---|---|---|---|
| | Modification | genome editing | |
| Modification(s) | location | efficiency | References |
| M | Terminal [symbol] | [symbol] | [symbol] |
| | | | [symbol] |
| MS | Terminal [symbol] | [symbol] | [symbol] |
| | | | [symbol] |
| | [symbol] | [symbol] | [symbol] |
| | | | [symbol] |
| | [symbol] | [symbol] | [symbol] |
| | [symbol] | | [symbol] |
| | [symbol] | [symbol] | [symbol] |
| MSP | [symbol] | [symbol] | [symbol] |
| [symbol] | [symbol] | [symbol] | [symbol] |
| | [symbol] | [symbol] | [symbol] |
| | [symbol] | | |
| 2′-F | [symbol] | [symbol] | [symbol] |
| | [symbol] | [symbol] | [symbol] |
| | [symbol] | [symbol] | [symbol] |
| | [symbol] | | |
| | [symbol] | [symbol] | [symbol] |
| | | | [symbol] |
| 2′-F + PS | [symbol] | [symbol] | [symbol] |
| | [symbol] | [symbol] | [symbol] |
| | [symbol] | | |
| | [symbol] | [symbol] | [symbol] |
| | [symbol] | [symbol] | [symbol] |
| | [symbol] | | |
| | [symbol] | | |
| PS | [symbol] | [symbol] | [symbol] |

[symbol]

[symbol]

indicates data missing or illegible when filed

Accordingly, in various embodiments of the present invention, the genome editing system comprising TnpB and further comprises one or more chemical modifications selected from, but not limited to the modifications in Table A.

In exemplary embodiments, chemical modifications to the reRNA include modifications on the ribose rings and phosphate backbone of reRNAs and modifications at the 2′OH include 2′-O-Me, 2′-F, and 2′F-ANA. More extensive ribose modifications include 2′F-4′-Cα-OMe and 2′, 4′-di-Cα-OMe combine modification at both the 2′ and 4′ carbons. Phosphodiester modifications include sulfide-based Phosphorothioate (PS) or acetate-based phosphonoacetate alterations. Combinations of the ribose and phosphodiester modifications have given way to formulations such as 2′-O-methyl 3′phosphorothioate (MS), or 2′-O-methyl-3′-thioPACE (MSP), and 2′-O-methyl-3′-phosphonoacetate (MP)RNAs. Locked and unlocked nucleotides such as locked nucleic acid (LNA), bridged nucleic acids (BNA), S-constrained ethyl (cEt), and unlocked nucleic acid (UNA) are examples of sterically hindered nucleotide modifications. Modifications to make a phosphodiester bond between the 2′ and 5′ carbons (2′, 5′-RNA) of adjacent RNAs as well as a butane 4-carbon chain link between adjacent RNAs have been described.

In embodiments involving configuring TnpB as a prime editor (e.g., by fusing TnpB to a reverse transcriptase), a reRNA can be modified by including a PE extension arm on the terminal end of the guide portion of the reRNA. Extension arms for generating pegRNAs for using with prime editors can be found described in the following references, each of which are incorporated by reference:

Prime editing was first described in Anzalone et al., “Search- and -replace genome editing without double-strand breaks or donor DNA,” Nature, December 2019, 576 (7789): pp. 149-157, which is incorporated herein in its entirety. Prime editing has subsequently been described and detailed in numerous follow-on publications, including, for example, (i) Liu et al., “Prime editing: a search and replace tool with versatile base changes,” Yi Chuan, Nov. 20, 2022, 44 (11): 993-1008; (ii) Lu C et al., “Prime Editing: An All-Rounder for Genome Editing. Int J Mol Sci. 2022 Aug. 30;23 (17): 9862; (iii) Velimirovic M, Zanetti L C, Shen M W, Fife J D, Lin L, Cha M, Akinci E, Barnum D, Yu T, Sherwood R I. Peptide fusion improves prime editing efficiency. Nat Commun. 2022 Jun. 18;13 (1): 3512. doi: 10.1038/s41467-022-31270-y. PMID: 35717416; PMCID: PMC9206660; (iv) Velimirovic M, Zanetti L C, Shen M W, Fife J D, Lin L, Cha M, Akinci E, Barnum D, Yu T, Sherwood R I. Peptide fusion improves prime editing efficiency. Nat Commun. 2022 Jun. 18;13 (1): 3512. doi: 10.1038/s41467-022-31270-y. PMID: 35717416; PMCID: PMC9206660; (v) Habib O, Habib G, Hwang G H, Bae S. Comprehensive analysis of prime editing outcomes in human embryonic stem cells. Nucleic Acids Res. 2022 Jan. 25;50 (2): 1187-1197. doi: 10.1093/nar/gkab1295. PMID: 35018468; PMCID: PMC8789035; (vi) Marzec M, Brąszewska-Zalewska A, Hensel G. Prime Editing: A New Way for Genome Editing. Trends Cell Biol. 2020 April;30 (4): 257-259. doi: 10.1016/j.tcb.2020.01.004. Epub 2020 Jan. 27. PMID: 32001098; (vii) Tao R, Wang Y, Jiao Y, Hu Y, Li L, Jiang L, Zhou L, Qu J, Chen Q, Yao S. Bi-PE: bi-directional priming improves CRISPR/Cas9 prime editing in mammalian cells. Nucleic Acids Res. 2022 Jun. 24;50 (11): 6423-6434. doi: 10.1093/nar/gkac506. PMID: 35687127; PMCID: PMC9226529; (viii)Nelson J W, Randolph P B, Shen S P, Everette K A, Chen P J, Anzalone A V, An M, Newby G A, Chen J C, Hsu A, Liu D R. Engineered pegRNAs improve prime editing efficiency. Nat Biotechnol. 2022 March;40 (3): 402-410. doi: 10.1038/s41587-021-01039-7. Epub 2021 Oct. 4. Erratum in: Nat Biotechnol. 2021 Dec. 8; PMID: 34608327; PMCID: PMC8930418; (ix) Doman J L, Sousa A A, Randolph P B, Chen P J, Liu D R. Designing and executing prime editing experiments in mammalian cells. Nat Protoc. 2022 November; 17 (11): 2431-2468. doi: 10.1038/s41596-022-00724-4. Epub 2022 Aug. 8. PMID: 35941224; PMCID: PMC9799714; (x) Jiao Y, Zhou L, Tao R, Wang Y, Hu Y, Jiang L, Li L, Yao S. Random-PE: an efficient integration of random sequences into mammalian genome by prime editing. Mol Biomed. 2021 Nov. 18;2 (1): 36. doi: 10.1186/s43556-021-00057-w. PMID: 35006470; PMCID: PMC8607425; and (xi) Awan MJA, Ali Z, Amin I, Mansoor S. Twin prime editor: seamless repair without damage. Trends Biotechnol. 2022 April;40 (4): 374-376. doi: 10.1016/j.tibtech.2022.01.013. Epub 2022 Feb. 10. PMID: 35153078, all of which are incorporated herein by reference.

In addition, the following references may be referred to when making and designing reRNAs that are modified with a PE extension arm for conducting prime editing.

| Publication No. | Publication Date | Title |
|---|---|---|
| WO 2023/015309 A2 | Feb. 9, 2023 | IMPROVED PRIME EDITORS AND |
| | | METHODS OF USE |
| WO 2023/004439 A2 | Jan. 26, 2023 | GENOME EDITING COMPOSITIONS AND |
| | | METHODS FOR TREATMENT OF CHRONIC |
| | | GRANULOMATOUS DISEASE |
| WO 2023/288332 A2 | Jan. 19, 2023 | GENOME EDITING COMPOSITIONS AND |
| | | METHODS FOR TREATMENT OF WILSON'S |
| | | DISEASE |
| WO 2023/283092 A1 | Jan. 12, 2023 | COMPOSITIONS AND METHODS FOR |
| | | EFFICIENT GENOME EDITING |
| WO 2023/283246 A1 | Jan. 12, 2023 | MODULAR PRIME EDITOR SYSTEMS FOR |
| | | GENOME ENGINEERING |
| WO 2022/256714 A3 | Jan. 12, 2023 | GENOME EDITING COMPOSITIONS AND |
| | | METHODS FOR TREATMENT OF WILSON'S |
| | | DISEASE |
| EP 4107273 A1 | Dec. 28, 2022 | PRIME EDITING TECHNOLOGY FOR |
| | | PLANT GENOME ENGINEERING |
| WO 2022/256714 A2 | Dec. 8, 2022 | GENOME EDITING COMPOSITIONS AND |
| | | METHODS FOR TREATMENT OF WILSON'S |
| | | DISEASE |
| WO 2022/234051 A1 | Nov. 10, 2022 | SPLIT PRIME EDITING ENZYME |
| US 2022/0356469 A1 | Nov. 10, 2022 | METHODS AND COMPOSITIONS FOR |
| | | EDITING NUCLEOTIDE SEQUENCES |
| | | METHODS AND COMPOSITIONS FOR |
| | | EDITING NUCLEOTIDE SEQUENCES |
| WO 2022/206352 A1 | Oct. 6, 2022 | PRIME EDITING TOOL, FUSION RNA, AND |
| | | USE THEREOF |
| WO 2022/212926 A1 | Oct. 6, 2022 | METHODS AND COMPOSITIONS FOR |
| | | EDITING NUCLEOTIDE SEQUENCES |
| WO 2022/204476 A1 | Sep. 29, 2022 | NUCLEOTIDE EDITING TO REFRAME DMD |
| | | TRANSCRIPTS BY BASE EDITING AND |
| | | PRIME EDITING |
| WO 2022/203905 A1 | Sep. 29, 2022 | PRIME EDITING-BASED SIMULTANEOUS |
| | | GENOMIC DELETION AND INSERTION |
| U.S. Pat. No. 11,447,770 B1 | Sep. 20, 2022 | Methods and compositions for prime editing |
| | | nucleotide sequences |
| WO 2022/174829 A1 | Aug. 25, 2022 | EDITING OF DOUBLE-STRANDED DNA |
| | | WITH RELAXED PAM REQUIREMENT |
| | | FIELD OF THE DISCLOSURE |
| WO 2022/170058 A1 | Aug. 11, 2022 | PRIME EDITOR SYSTEM FOR IN VIVO |
| | | GENOME EDITING |
| WO 2022/169235 A1 | Aug. 11, 2022 | PRIME EDITING COMPOSITION WITH |
| | | IMPROVED EDITING EFFICIENCY |
| WO 2022/150790 A3 | Aug. 11, 2022 | PRIME EDITOR VARIANTS, CONSTRUCTS, |
| | | AND METHODS FOR ENHANCING PRIME |
| | | EDITING EFFICIENCY AND PRECISION |
| WO 2022/149166 A1 | Jul. 14, 2022 | A COCKTAIL FORMULATION FOR |
| | | SELECTIVE ENRICHMENT OF GENE- |
| | | MODIFIED CELLS |
| WO 2022/150790 A2 | Jul. 14, 2022 | PRIME EDITOR VARIANTS, CONSTRUCTS, |
| | | AND METHODS FOR ENHANCING PRIME |
| | | EDITING EFFICIENCY AND PRECISION |
| U.S. Pat. No. 11,384,353 B2 | Jul. 12, 2022 | Inhibition of unintended mutations in gene |
| | | editing |
| WO 2022/067130 A3 | Jun. 23, 2022 | PRIME EDITING GUIDE RNAS, |
| | | COMPOSITIONS THEREOF, AND |
| | | METHODS OF USING THE SAME |
| WO 2022/114815 A1 | Jun. 2, 2022 | COMPOSITION FOR PRIME EDITING |
| | | COMPRISING TRANS-SPLICING ADENO- |
| | | ASSOCIATED VIRUS VECTOR |
| WO 2022/100662 A1 | May 19, 2022 | GENOMIC EDITING OF IMPROVED |
| | | EFFICIENCY AND ACCURACY |
| WO 2022/098765 A1 | May 12, 2022 | SPLIT PRIME EDITING PLATFORMS |
| WO 2022/098885 A1 | May 12, 2022 | PRECISE GENOME DELETION AND |
| | | REPLACEMENT METHOD BASED ON |
| | | PRIME EDITING |
| WO 2022/071745 A1 | Apr. 7, 2022 | PRIME EDITING USING HIV REVERSE |
| | | TRANSCRIPTASE AND CAS9 OR VARIANT |
| | | THEREOF |
| WO 2022/067130 A2 | Mar. 31, 2022 | PRIME EDITING GUIDE RNAS, |
| | | COMPOSITIONS THEREOF, AND |
| | | METHODS OF USING THE SAME |
| WO 2022/065689 A1 | Mar. 31, 2022 | PRIME EDITING-BASED GENE EDITING |
| | | COMPOSITION WITH ENHANCED EDITING |
| | | EFFICIENCY AND USE THEREOF |
| US 2022/0064626 A1 | Mar. 3, 2022 | INHIBITION OF UNINTENDED |
| | | MUTATIONS IN GENE EDITING |
| WO 2022/032085 A1 | Feb. 10, 2022 | TARGETED SEQUENCE INSERTION |
| | | COMPOSITIONS AND METHODS |
| WO 2022/025623 A1 | Feb. 3, 2022 | SYSTEM AND METHOD FOR PRIME |
| | | EDITING EFFICIENCY PREDICTION USING |
| | | DEEP LEARNING |
| WO 2021/226558 A8 | Jan. 13, 2022 | METHODS AND COMPOSITIONS FOR |
| | | SIMULTANEOUS EDITING OF BOTH |
| | | STRANDS OF A TARGET DOUBLE- |
| | | STRANDED NUCLEOTIDE SEQUENCE |
| WO 2021/243289 A1 | Dec. 2, 2021 | SYSTEMS AND METHODS FOR STABLE |
| | | AND HERITABLE ALTERATION BY |
| | | PRECISION EDITING (SHAPE) |
| WO 2021/226558 A1 | Nov. 11, 2021 | METHODS AND COMPOSITIONS FOR |
| | | SIMULTANEOUS EDITING OF BOTH |
| | | STRANDS OF A TARGET DOUBLE- |
| | | STRANDED NUCLEOTIDE SEQUENCE |
| WO 2021/215897 A1 | Oct. 28, 2021 | GENOME EDITION USING CAS9 OR CAS9 |
| | | VARIANT |
| WO 2021/215827 A1 | Oct. 28, 2021 | GENOME EDITING USING CAS9 OR CAS9 |
| | | VARIANT |
| WO 2020/191248 A8 | Oct. 21, 2021 | METHODS AND COMPOSITIONS FOR |
| | | EDITING NUCLEOTIDE SEQUENCES |
| WO 2020/191234 A8 | Oct. 21, 2021 | METHODS AND COMPOSITIONS FOR |
| | | EDITING NUCLEOTIDE SEQUENCES |
| WO 2021/165508 A1 | Aug. 26, 2021 | PRIME EDITING TECHNOLOGY FOR |
| | | PLANT GENOME ENGINEERING |
| WO 2021/138469 A1 | Jul. 8, 2021 | GENOME EDITING USING REVERSE |
| | | TRANSCRIPTASE ENABLED AND FULLY |
| | | ACTIVE CRISPR COMPLEXES |
| WO 2021/092204 A1 | May 14, 2021 | METHODS AND COMPOSITIONS FOR |
| | | NUCLEIC ACID-GUIDED NUCLEASE CELL |
| | | TARGETING SCREEN |
| WO 2021/076876 A1 | Apr. 22, 2021 | GENOTYPING EDITED MICROBIAL |
| | | STRAINS |
| WO 2021/072328 A1 | Apr. 15, 2021 | METHODS AND COMPOSITIONS FOR |
| | | PRIME EDITING RNA |
| WO 2020/191153 A8 | Dec. 30, 2020 | METHODS AND COMPOSITIONS FOR |
| | | EDITING NUCLEOTIDE SEQUENCES |
| WO 2020/191153 A3 | Dec. 10, 2020 | METHODS AND COMPOSITIONS FOR |
| | | EDITING NUCLEOTIDE SEQUENCES |
| WO 2020/191153 A9 | Nov. 12, 2020 | METHODS AND COMPOSITIONS FOR |
| | | EDITING NUCLEOTIDE SEQUENCES |
| WO 2020/191171 A9 | Oct. 29, 2020 | METHODS AND COMPOSITIONS FOR |
| | | EDITING NUCLEOTIDE SEQUENCES |
| WO 2020/191248 A1 | Sep. 24, 2020 | METHOD AND COMPOSITIONS FOR |
| | | EDITING NUCLEOTIDE SEQUENCES |
| WO 2020/191239 A1 | Sep. 24, 2020 | METHODS AND COMPOSITIONS FOR |
| | | EDITING NUCLEOTIDE SEQUENCES |
| WO 2020/191153 A2 | Sep. 24, 2020 | METHODS AND COMPOSITIONS FOR |
| | | EDITING NUCLEOTIDE SEQUENCES |
| WO 2020/191246 A1 | Sep. 24, 2020 | METHODS AND COMPOSITIONS FOR |
| | | EDITING NUCLEOTIDE SEQUENCES |
| WO 2020/191249 A1 | Sep. 24, 2020 | METHODS AND COMPOSITIONS FOR |
| | | EDITING NUCLEOTIDE SEQUENCES |
| WO 2020/191233 A1 | Sep. 24, 2020 | METHODS AND COMPOSITIONS FOR |
| | | EDITING NUCLEOTIDE SEQUENCES |
| WO 2020/191243 A1 | Sep. 24, 2020 | METHODS AND COMPOSITIONS FOR |
| | | EDITING NUCLEOTIDE SEQUENCES |
| WO 2020/191234 A1 | Sep. 24, 2020 | METHODS AND COMPOSITIONS FOR |
| | | EDITING NUCLEOTIDE SEQUENCES |
| WO 2020/191245 A1 | Sep. 24, 2020 | METHODS AND COMPOSITIONS FOR |
| | | EDITING NUCLEOTIDE SEQUENCES |
| WO 2020/191242 A1 | Sep. 24, 2020 | METHODS AND COMPOSITIONS FOR |
| | | EDITING NUCLEOTIDE SEQUENCES |
| WO 2020/191171 A1 | Sep. 24, 2020 | METHODS AND COMPOSITIONS FOR |
| | | EDITING NUCLEOTIDE SEQUENCES |
| WO 2020/191241 A1 | Sep. 24, 2020 | METHODS AND COMPOSITIONS FOR |
| | | EDITING NUCLEOTIDE SEQUENCES |
| WO 2020/156575 A1 | Aug. 6, 2020 | INHIBITION OF UNINTENDED |
| | | MUTATIONS IN GENE EDITING |
| U.S. Pat. No. 10,189,831 B2 | Jan. 29, 2019 | Non-nucleoside reverse transcriptase inhibitors |
| WO 2019/014564 A1 | Jan. 17, 2019 | SYSTEMS AND METHODS FOR TARGETED |
| | | INTEGRATION AND GENOME EDITING |
| | | AND DETECTION THEREOF USING |
| | | INTEGRATED PRIMING SITES |
| U.S. Pat. No. 10,150,955 B2 | Dec. 11, 2018 | Stabilized reverse transcriptase fusion proteins |
| WO 2018/049168 A1 | Mar. 15, 2018 | HIGH-THROUGHPUT PRECISION GENOME |
| | | EDITING |
| U.S. Pat. No. 9,783,791 B2 | Oct. 10, 2017 | Mutant reverse transcriptase and methods of use |
| U.S. Pat. No. 9,458,484 B2 | Oct. 4, 2016 | Reverse transcriptase mixtures with improved |
| | | storage stability |

### Aptamers

In particular embodiments, the compositions or complexes have a reRNA component molecule with a functional structure designed to improve reRNA component molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Sarnie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the reRNA component molecule is modified, e.g., by one or more aptamer(s) designed to improve reRNA component molecule delivery, including delivery across the cellular membrane, to intracellular compartments, or into the nucleus. Such a structure can include, either in addition to the one or more aptamer(s) or without such one or more aptamer(s), moiety(ies) so as to render the nucleic acid component molecule deliverable, inducible or responsive to a selected effector. The invention accordingly comprehends a reRNA component molecule that responds to normal or pathological physiological conditions, including without limitation pH, hypoxia, oxygen concentration, temperature, protein concentration, enzymatic concentration, lipid structure, light exposure, mechanical disruption (e.g. ultrasound waves), magnetic fields, electric fields, or electromagnetic radiation.

### Target Adjacent Motifs (TAMs)

The TnpB systems disclosed herein may recognize a target adjacent motif (TAM) in order to recognize and bind a target sequence on a target sequence. In one embodiment, the nucleic acid-guided nucleases and related compositions do not contain a TAM requirement. The precise sequence and length requirements for the TAM will differ depending on the nucleic acid-guided nucleases used. In some examples, TAMs are typically 2-5 base pair sequences adjacent the protospacer. In one example embodiment, the TAM is 3′ adjacent to the target polynucleotide. In another example embodiment, the TAM is 5′ adjacent to the target sequence of the target polynucleotide.

In one embodiment, the cleavage site is distant from the TAM, e.g., the cleavage occurs after the nth nucleotide on the non-target strand and after the nucleotide on the targeted strand. In one embodiment, the cleavage site occurs after an identified nucleotide (counted from the TAM) on the non-target strand and after the further identified nucleotide (counted from the TAM) on the targeted strand. In one embodiment, a vector encodes a nucleic acid-targeting effector protein that may be mutated with respect to a corresponding wild-type enzyme such that the mutated nucleic acid-targeting effector protein lacks the ability to cleave one or both DNA and RNA strands of a target polynucleotide containing a target sequence.

### Donor Templates

In one embodiment, the compositions and systems herein may further comprise one or more donor templates for use in homology-directed repair mediated editing. In some cases, the donor template may comprise one or more polynucleotides. In certain cases, the donor template may comprise coding sequences for one or more polynucleotides. The donor template may be a DNA template. It may be single stranded or double stranded. It may also be circular single or double stranded. It may also be linear single stranded or double stranded.

In one embodiment, FIG. 1C shows an LNP that comprises a TnpB gene editing system described herein. The LNP comprises a TnpB ncRNA (which includes a guide RNA) and a coding RNA that encodes a TnpB and optionally one or more accessory proteins. The LNP in certain embodiments may also comprise a donor template.

The donor template may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor template alters a stop codon in the target polynucleotide. For example, the donor template may correct a premature stop codon. The correction may be achieved by deleting the stop codon or introduces one or more mutations to the stop codon. In other example embodiments, the donor template addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g. sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor templates that encode gene or gene fragments that compensate for or override defective gene expression such that cell phenotypes associated with defective gene expression are eliminated or changed to a different or desired cellular phenotype.

In an embodiment of the invention, the donor template may include, but not be limited to, genes or gene fragments, encoding proteins or RNA transcripts to be expressed, regulatory elements, repair templates, and the like. According to the invention, the donor templates may comprise left end and right end sequence elements that function with transposition components that mediate insertion.

In certain cases, the donor template manipulates a splicing site on the target polynucleotide. In some examples, the donor template disrupts a splicing site. The disruption may be achieved by inserting the polynucleotide to a splicing site and/or introducing one or more mutations to the splicing site. In certain examples, the donor template may restore a splicing site. For example, the polynucleotide may comprise a splicing site sequence.

The donor template to be inserted may has a size from 10 base pair or nucleotides to 50 kb in length, e.g., from 50 to 40 k, from 100 and 30 k, from 100 to 10000, from 100 to 300, from 200 to 400, from 300 to 500, from 400 to 600, from 500 to 700, from 600 to 800, from 700 to 900, from 800 to 1000, from 900 to from 1100, from 1000 to 1200, from 1100 to 1300, from 1200 to 1400, from 1300 to 1500, from 1400 to 1600, from 1500 to 1700, from 600 to 1800, from 1700 to 1900, from 1800 to 2000 base pairs (bp) or nucleotides in length.

In some embodiments, the heterologous nucleic acid sequence is a donor DNA template that can be integrated into a host genome via HDR.

In certain embodiments, the heterologous nucleic acid comprises or encodes a donor/template sequence, wherein the donor/template corrects/repairs/removes a mutation at the target genome site. For example, the mutation may be a mutated exon in a disease gene.

In certain embodiments, the donor/template may encode or comprises a functional DNA element, such as a promoter, an enhancer, a protein binding sequence, a methylation site, or a homology region for assisting gene editing, etc.

By “donor DNA” or “donor DNA template” it is meant a single-stranded DNA to be inserted at a site cleaved by a gene-editing nuclease (e.g., a TnpB nuclease)(e.g., after dsDNA cleavage, after nicking a target DNA, after dual nicking a target DNA, and the like). The donor DNA template can contain sufficient homology to a genomic sequence at the target site, e.g., 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology.

Approximately 25, 50, 100, or 200 nucleotides, or more than 200 nucleotides, of sequence homology between a donor DNA template and a genomic sequence (or any integral value between 10 and 200 nucleotides, or more) can support homology-directed repair. Donor DNA template can be of any length, e.g., 50 nucleotides or more, 100 nucleotides or more, 250 nucleotides or more, 500 nucleotides or more, 1000 nucleotides or more, 5000 nucleotides or more, etc. A suitable donor DNA template can be from 50 nucleotides to 100 nucleotides, from 100 nucleotides to 500 nucleotides, from 500 nucleotides to 1000 nucleotides, from 1000 nucleotides to 5000 nucleotides, or from 5000 nucleotides to 10,000 nucleotides, or more than 10,000 nucleotides, in length.

As noted above, the donor DNA template comprises a first homology arm and a second homology arm. The first homology arm is at or near 5′ end of the donor DNA; and comprises a nucleotide sequence that is at least partially complementary to a first nucleotide sequence in a target nucleic acid. The second homology arm is at or near 3′ end of the donor DNA; and comprises a nucleotide sequence that is at least partially complementary to a second nucleotide sequence in the target nucleic acid. The first and second homology arms can each independently have a length of from about 10 nucleotides to 400 nucleotides; e.g., from 10 nucleotides (nt) to 15 nt, from 15 nt to 20 nt, from 20 nt to 25 nt, from 25 nt to 30 nt, from 30 nt to 35 nt, from 35 nt to 40 nt, from 40 nt to 45 nt, from 45 nt to 50 nt, from 50 nt to 75 nt, from 75 nt to 100 nt, from 100 nt to 125 nt, from 125 nt to 150 nt, from 150 nt to 175 nt, from 175 nt to 200 nt, from 200 nt to 225 nt, from 225 nt to 250 nt, from 250 nt to 275 nt, from 275 nt to 300 nt, from 325 nt to 350 nt, from 350 nt to 375 nt, or from 375 nt to 400 nt.

In certain embodiments, the donor DNA template is used for editing the target nucleotide sequence. In certain embodiments, the donor DNA template comprises one or more mutations to be introduced into the target polynucleotide. Examples of such mutations include substitutions, deletions, insertions, or a combination thereof. In certain embodiments, the mutation causes a shift in an open reading frame on the target polynucleotide. In certain embodiments, the donor polynucleotide alters a stop codon in the target polynucleotide. In certain embodiments, the donor polynucleotide corrects a premature stop codon. The correction can be achieved by deleting the stop codon, or by introducing one or more sequence changes to alter the stop codon to a codon. In certain embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment includes a fragment less than the entire copy of a gene but otherwise provides sufficient nucleotide sequence to restore the functionality of a wild type gene or non-coding regulatory sequence (e.g., sequences encoding long non-coding RNA).

In certain embodiments, the donor DNA template may be used to replace a single allele of a defective gene or defective fragment thereof. In another embodiment, the donor DNA template is used to replace both alleles of a defective gene or defective gene fragment. A “defective gene” or “defective gene fragment” is a gene or portion of a gene that when expressed, fails to generate a functioning protein or non-coding RNA with functionality of the corresponding wild-type gene.

In certain example embodiments, these defective genes may be associated with one or more disease phenotypes. In certain example embodiments, the defective gene or gene fragment is not replaced but the heterologous nucleic acid is used to insert donor polynucleotides that encode gene or gene fragments that compensate for or override defective gene expression such that cell phenotypes associated with defective gene expression are eliminated or changed to a different or desired cellular phenotype. This can be achieved by including the coding sequence of a therapeutic protein, such as a therapeutic antibody or functional fragment thereof, or a wild-type version of a defective protein associated with one or more disease phenotypes.

In certain embodiments, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain embodiments, the donor DNA template manipulates a splicing site on the target polynucleotide. In certain embodiments, the donor DNA template disrupts a splicing site. The disruption may be achieved by inserting the polynucleotide to a splicing site and/or introducing one or more mutations to the splicing site. In certain embodiments, the donor polynucleotide may restore a splicing site. For example, the polynucleotide may comprise a splicing site sequence.

In certain embodiments, the donor DNA template to be inserted has a size from 10 bp to 50 kb in length, e.g., from 50 bp to ˜40 kb, from 100 bp to ˜30 kb, from 100 bp to ˜10 kb, from 100 bp to 300 bp, from 200 bp to 400 bp, from 300 bp to 500 bp, from 400 bp to 600 bp, from 500 bp to 700 bp, from 600 bp to 800 bp, from 700 bp to 900 bp, from 800 bp to 1000 bp, from 900 bp to 1100 bp, from 1000 bp to 1200 bp, from 1100 bp to 1300 bp, from 1200 bp to 1400 bp, from 1300 bp to 1500 bp, from 1400 bp to 1600 bp, from 1500 bp to 1700 bp, from 1600 bp to 1800 bp, from 1700 bp to 1900 bp, from 1800 bp to 2000 bp nucleotides in length.

In certain embodiments, the homologous arm on one or both ends of the sequence to be inserted is independently about 20 bp, 40 bp, 60 bp, 80 bp, 100 bp, 120 bp, or 150 bp.

The first homology arm and the second homology arm of the donor DNA flank a nucleotide sequence (“a nucleotide sequence of interest” or “an intervening nucleotide sequence”) that is to be introduced into a target nucleic acid. The nucleotide sequence of interest can comprise: i) a nucleotide sequence encoding a polypeptide of interest; ii) a nucleotide sequence encoding an exon of a gene; iii) a promoter sequence; iv) an enhancer sequence; v) a nucleotide sequence encoding a non-coding RNA; or vi) any combination of the foregoing.

The donor DNA can provide for gene correction, gene replacement, gene tagging, transgene insertion, nucleotide deletion, gene disruption, gene mutation, etc. For example, the donor DNA can be used to add, e.g., insert or replace, nucleic acid material to a target DNA (e.g. to “knock in” a nucleic acid that encodes a protein, an siRNA, an miRNA, etc.), to add a tag (e.g., 6xHis, a fluorescent protein (e.g., a green fluorescent protein; a yellow fluorescent protein, etc.), hemagglutinin (HA), FLAG, etc.), to add a regulatory sequence to a gene (e.g. promoter, polyadenylation signal, internal ribosome entry sequence (IRES), 2A peptide, start codon, stop codon, splice signal, localization signal, enhancer, etc.), to modify a nucleic acid sequence (e.g., introduce a mutation), and the like. For example, the donor DNA can be used to modify DNA in a site-specific, i.e. “targeted”, way; for example gene knock-out, gene knock-in, gene editing, gene tagging, etc., as used in, for example, gene therapy, e.g. to treat a disease; or as an antiviral, antipathogenic, or anticancer therapeutic, the production of genetically modified organisms in agriculture, the large scale production of proteins by cells for therapeutic, diagnostic, or research purposes, the induction of pluripotent stem cells, biological research, the targeting of genes of pathogens for deletion or replacement, etc.

In some cases, the donor DNA comprises a nucleotide sequence encoding a polypeptide of interest. Polypeptides of interest include, e.g., a) functional versions of a polypeptide that comprises one or more amino acid substitutions, insertions, and/or deletions and that exhibits reduced function, e.g., where the reduced function is associated with or causes a pathological condition; b) fluorescent polypeptides; c) hormones; d) receptors for ligands; e) ion channels; f) neurotransmitters; g) and the like.

In some cases, the donor DNA comprises a nucleotide sequence that encodes a wild-type protein that is lacking in the recipient cell. In some cases, the donor DNA encodes a wild type factor (e.g. Factor VII, Factor VIII, Factor IX and the like) involved in coagulation. In some cases, the donor DNA comprises a nucleotide sequence that encodes a therapeutic antibody. In some cases, the donor DNA comprises a nucleotide sequence that encodes an engineered protein or receptor. In some cases, the engineered receptor is a T cell receptor (TCR), a natural killer (NK) receptor (NKR), or a B cell receptor (BCR). In some cases, the engineered TCR or NKR targets a cancer marker (e.g., a polypeptide that is expressed (e.g., over-expressed) on the surface of a cancer cell). In some cases, the donor DNA comprises a nucleotide sequence that encodes a chimeric antigen receptor (CAR). In some cases, the CAR targets a cancer marker. Donor DNAs encoding CAR, TCR, and/or NCR proteins may be folded into DNA origami structures (DNA nanostructures) and delivered into T cells or NK cells in vitro or in vivo.

Non-limiting examples of polypeptides that can be encoded by a donor DNA include, e.g., IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJ5 (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCG5 (ATP-binding cassette, sub-family G (WHITE), member 5), PRDX2 (peroxiredoxin 2), CAPN5 (calpain 5), PARP14 (poly (ADP-ribose) polymerase family, member 14), MEX3C(mex-3 homolog C(C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN(statin), SERPINE1 (serpin peptidase inhibitor, clade E (nexin, plasminogen activator inhibitor type 1), member 1), ALB (albumin), ADIPOQ (adiponectin, C₁Q and collagen domain containing), APOB (apolipoprotein B (including Ag (x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN(renin), PPARA (peroxisome proliferator-activated receptor alpha), PON1 (paraoxonase 1), KNG1 (kininogen 1), CCL2 (chemokine (C-C motif) ligand 2), LPL (lipoprotein lipase), vWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAM1 (vascular cell adhesion molecule 1), IFNG (interferon, gamma), LPA (lipoprotein, Lp (a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric Golgi complex 2), MMP9 (matrix metallopeptidase 9 (gelatinase B, 92 kDa gelatinase, 92 kDa type IV collagenase)), SERPINC1 (serpin peptidase inhibitor, clade C (antithrombin), member 1), F8 (coagulation factor VIII, procoagulant component), HMOX1 (heme oxygenase (decycling) 1), APOC3 (apolipoprotein C-III), IL8 (interleukin 8), PROK1 (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), ILIA (interleukin 1, alpha), RETN(resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein 111a, antigen CD61)), CAT (catalase), UTS2 (urotensin 2), THBD (thrombomodulin), F10 (coagulation factor X), CP (ceruloplasmin (ferroxidase)), TNFRSF11B (tumor necrosis factor receptor superfamily, member lib), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRB1 (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDE5A (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCR5 (chemokine (C-C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIFIA (hypoxia inducible factor 1, alpha subunit (basic helix-loop-helix transcription factor)), CXCR4 (chemokine (C-X-C motif) receptor 4), PROC (protein C(inactivator of coagulation factors Va and Villa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid Al), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomer ase G (cyclophilin G)), ILIR1 (interleukin 1 receptor, type I), AR (androgen receptor), CYP1A1 (cytochrome P450, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, pl6, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CAB INI (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAVI (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp 1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member Al), CX3CR1 (chemokine (C-X3-C motif) receptor 1), TH (tyrosine hydroxylase), F9 (coagulation factor IX), GHI (growth hormone 1), TF (transferrin), HFE (hemochromatosis), IE17A (interleukin 17A), PTEN (phosphatase and tensin homolog), GSTM1 (glutathione S-transferase mu 1), DMD (dystrophin), GATA4 (GATA binding protein 4), F13A1 (coagulation factor XIII, Al polypeptide), TTR (transthyretin), FABP4 (fatty acid binding protein 4, adipocyte), PON3 (paraoxonase 3), APOC1 (apolipoprotein C-I), INSR (insulin receptor), TNFRSF1B (tumor necrosis factor receptor superfamily, member IB), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THBS1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCH1 (Notch homolog 1, translocation-associated (Drosophila)), UGT1A1 (UDP glucuronosyltransferase 1 family, polypeptide Al), IFNA1 (interferon, alpha 1), PPARD (peroxisome proliferator-activated receptor delta), SIRT1 (sirtuin (silent mating type information regulation 2 homolog) 1(S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione S-transferase theta 1), IL6ST (interleukin 6 signal transducer (gp130, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member Al (aldehyde reductase)), ALDH9A1 (aldehyde dehydrogenase 9 family, member Al), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREB1 (cAMP responsive element binding protein 1), POMC (proopiomelanocortin), RAC1 (ras-related C3 botulinum toxin substrate 1 (rho family, small GTP binding protein Rac1)), LMNA (lamin NC), CD59 (CD59 molecule, complement regulatory protein), SCN5A (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin LI), PCNA (proliferating cell nuclear antigen), IGF2 (insulin like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C-X-C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COL1A2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C-C motif) ligand 5), IRF1 (interferon regulatory factor 1), CFLAR (CASP8 and FADD-like apoptosis regulator), CALC A (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C-C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCHI (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PEC AMI (platelet/endothelial cell adhesion molecule), CCL4 (chemokine (C-C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PROS1 (protein S (alpha)), CTF1 (cardiotrophin 1), SGCB (sarcoglycan, beta (43 kDa dystrophin-associated glycoprotein)), YMEIL1 (YMEI-like 1(S. cerevisiae)), CAMP (cathelicidin antimicrobial peptide), ZC3H12A (zinc finger CCCH-type containing 12A), AKRIB1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-b-methyltransf erase), S100B (S100 calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C-C motif) ligand 11), PGF (placental growth factor), THPO (thrombopoietin), GP6 (glycoprotein VI (platelet)), TACRI (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADHIC (alcohol dehydrogenase 1C(class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylate synthetase), SHC1 (SHC(Src homology 2 domain containing) transforming protein 1), LRP1 (low density lipoprotein receptor-related protein 1), SOCS3 (suppressor of cytokine signaling 3), ADH1B (alcohol dehydrogenase IB (class I), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity 111a, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor HI), NR112 (nuclear receptor subfamily 1, group I, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLPIR (glucagon-like peptide 1 receptor), GHR (growth hormone receptor), GSR (glutathione reductase), NQO1 (NAD (P) H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity Ila, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C-X-C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPA1A (heat shock 70 kDa protein 1A), FASN (fatty acid synthase), FGF1 (fibroblast growth factor 1 (acidic)), Fll (coagulation factor XI), ATP7A (ATPase, Cu++transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCF1E (butyrylcholinesterase), LIPE (lipase, hormone-sensitive), PRDX5 (peroxiredoxin 5), ADORA1 (adenosine Al receptor), WRN (Werner syndrome, RecQ helicase-like), CXCR3 (chemokine (C-X-C motif) receptor 3), CD81 (CD81 molecule), SMAD7 (SMAD family member 7), LAMC2 (laminin, gamma 2), MAP3K5 (mitogen-activated protein kinase kinase kinase 5), CF1GA (chromogranin A (parathyroid secretory protein 1)), IAPP (islet amyloid polypeptide), RFIO (rhodopsin), ENPP1 (ectonucleotide pyrophosphatase/phosphodiesterase 1), PTF1LF1 (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha), F2RL3 (coagulation factor II (thrombin) receptor-like 3), CX3CL1 (chemokine (C-X3-C motif) ligand 1), BDKRB1 (bradykinin receptor B1), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISFI (cytokine inducible SF12-containing protein), GAST (gastrin), MYOC (myocilin, trabecular mesh work inducible glucocorticoid response), ATP1A2 (ATPase, Na+/K+transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), F1SF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), delta 1), CTF1 (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOF1 (apolipoprotein FI (beta-2-glycoprotein I)), S100A8 (S100 calcium binding protein A8), IL11 (interleukin 11), ALOX15 (arachidonate 15-lipoxygenase), FBLN1 (fibulin 1), NR1F13 (nuclear receptor subfamily 1, group FI, member 3), SCD (stearoyl-CoA desaturase (delta-9-desaturase)), GIP (gastric inhibitory polypeptide), CF1 GB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), F1SD11B2 (hydroxy steroid (11-beta) dehydrogenase 2), CALCRL (calcitonin receptor-like), GALNT2 (UDP-N-acetyl-alpha-D-galactosamine: polypeptide N-acetylgalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adeno virus E1B 19 kDa interacting protein 3), GCKR (glucokinase (hexokinase 4) regulator), S100A12 (S100 calcium binding protein A 12), PADI4 (peptidyl arginine deaminase, type IV), HSPA14 (heat shock 70 kDa protein 14), CXCR1 (chemokine (C-X-C motif) receptor 1), H19 (H19, imprinted maternally expressed transcript (non-protein coding)), KRTAP19-3 (keratin associated protein 19-3), insulin, RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNAIC (calcium channel, voltage-dependent, L type, alpha 1C subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C(myocyte enhancer factor 2C), MAPKAPK2 (mitogen-activated protein kinase-activated protein kinase 2), TNFRSF11 A (tumor necrosis factor receptor superfamily, member 11a, NFKB activator), HSPA9 (heat shock 70 kDa protein 9 (mortalin)), CYSLTR1 (cysteinyl leukotriene receptor 1), MATIA (methionine adenosyltransferase I, alpha), OPRLI (opiate receptor-like 1), IMPA1 (inositol (myo)-1 (or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDHIB1 (aldehyde dehydrogenase 1 family, member B1), PARP2 (poly (ADP-ribose) polymerase 2), STAR (steroidogenic acute regulatory protein), LBP (lipopolysaccharide binding protein), ABCC6 (ATP-binding cassette, sub-family C(CFTR/MRP), member 6), RGS2 (regulator of G-protein signaling 2, 24 kDa), EFNB2 (ephrin-B2), cystic fibrosis transmembrane conductance regulator (CFTR), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C-C motif) receptor 6), GJB3 (gap junction protein, beta 3, 31 kDa), ILIRL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F11R (Fll receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), K′HK (ketohexokinase (fructokinase)), SAT1 (spermidine/spermine Nl-acetyltransferase 1), GGFI (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2 A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LIM and senescent cell antigen-like domains 1), RFIOB (ras homolog gene family, member B), LY96 (lymphocyte antigen 96), FOXO1 (forkhead box 01), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1(Drosophila)), PBX4 (pre-B-cell leukemia homeobox 4), NUPR1 (nuclear protein, transcriptional regulator, 1), 15-Sep (15 kDa selenoprotein), CILP2 (cartilage intermediate layer protein 2), TERC (telomerase RNA component), GGT2 (gamma-glutamyltransf erase 2), MT-CO1 (mitochondrially encoded cytochrome c oxidase I), UOX (urate oxidase, pseudogene), a CRISPR/Cas effector polypeptide, an enzymatically active CRISPR/Cas effector polypeptide (e.g., is capable of cleaving a target nucleic acid) and a CRISPR/Cas effector polypeptide that is not enzymatically active (e.g., does not cleave a target nucleic acid, but retains binding to the target nucleic acid). In some cases, the donor DNA encodes a wild-type version of any of the foregoing polypeptides; i.e., the donor DNA can encode a “normal” version that does not include a mutation(s) that results in reduced function, lack of function, or pathogenesis.

In some cases, the donor DNA comprises a nucleotide sequence encoding a fluorescent polypeptide. Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2 (12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, m PI urn (Shaner et al. (2005)Nat. Methods 2:905-909), and the like. Any of a variety of fluorescent and colored proteins from Anthozoan species, as described in, e.g., Matz et al. (1999)Nature Biotechnol. 17:969-973, can be encoded.

In some cases, the donor DNA encodes an RNA, e.g., an siRNA, a microRNA, a short hairpin RNA (shRNA), an anti-sense RNA, a riboswitch, a ribozyme, an aptamer, a ribosomal RNA, a transfer RNA, and the like.

A donor DNA can include, in addition to a nucleotide sequence encoding one or more gene products (e.g., an RNA and/or a polypeptide), one or more transcriptional control elements, e.g., a promoter, an enhancer, and the like. In some cases, the transcriptional control element is inducible. In some cases, the promoter is reversible. In some cases, the transcriptional control element is constitutive. In some cases, the promoter is functional in a eukaryotic cell. In some cases, the promoter is a cell type-specific promoter. In some cases, the promoter is a tissue-specific promoter.

The nucleotide sequence of the donor DNA is typically not identical to the target nucleic acid (e.g., genomic sequence) that it replaces. Rather, the donor DNA may contain at least one or more single base changes, insertions, deletions, inversions or rearrangements with respect to the target nucleic acid (e.g., genomic sequence), so long as sufficient homology is present to support homology-directed repair (e.g., for gene correction, e.g., to convert a disease-causing base pair or a non-disease-causing base pair). In some cases, the donor DNA comprises a non-homologous sequence flanked by two regions of homology, such that homology-directed repair between the target DNA region and the two flanking sequences results in insertion of the non-homologous sequence at the target region. Donor DNA may also comprise a vector backbone containing sequences that are not homologous to the DNA region of interest (the target nucleic acid) and that are not intended for insertion into the DNA region of interest (the target nucleic acid). Generally, the homologous region(s) of a donor sequence will have at least 50% sequence identity to a target nucleic acid (e.g., a genomic sequence) with which recombination is desired. In certain cases, 60%, 70%, 80%, 90%, 95%, 98%, 99%, or 99.9% sequence identity is present. Any value between 1% and 100% sequence identity can be present, depending upon the length of the donor polynucleotide.

The donor DNA may comprise certain nucleotide sequence differences as compared to the target nucleic acid (e.g., genomic sequence), where such difference include, e.g. restriction sites, nucleotide polymorphisms, selectable markers (e.g., drug resistance genes, fluorescent proteins, enzymes etc.), etc., which may be used to assess for successful insertion of the donor DNA at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence. In some cases, the donor DNA will include one or more nucleotide sequences to aid in localization of the donor to the nucleus of the recipient cell or to aid in the integration of the donor DNA into the target nucleic acid. For example, in some case, the donor DNA may comprise one or more nucleotide sequences encoding one or more nuclear localization signals (e.g. PKKKRKV (SEQ ID NO: 398), VSRKRPRP (SEQ ID NO: 399), QRKRKQ (SEQ ID NO: 400), and the like (Frietas et al (2009)Cun-Genomics 10:550-7). In some cases, the donor DNA will include nucleotide sequences to recruit DNA repair enzymes to increase insertion efficiency. Fiuman enzymes involved in homology directed repair include MRN-CtIP, BLM-DNA2, Exol, ERCC1, Rad51, Rad52, Ligase 1, RoIQ, PARP1, Ligase 3, BRCA2, RecQ/BLM-ToroIlla, RTEL, R°ïd, and Roth (Verma and Greenburg (2016) Genes Dev. 30 (10): 1138-1154). In some cases, the donor DNA is delivered as reconstituted chromatin (Cruz-Becerra and Kadonaga (2020) eLife 2020; 9: e55780 DOI: 10.7554/eLife.55780).

In some cases, the ends of the donor DNA are protected (e.g., from exonucleolytic degradation) by any convenient method and such methods are known to those of skill in the art. For example, one or more dideoxynucleotide residues can be added to the 3′ terminus of a linear molecule and/or self complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor DNA, additional lengths of sequence may be included outside of the regions of homology that can be degraded without impacting recombination.

### DNA-Repair Modulating Agents

In certain embodiments, the engineered TnpB systems described herein (e.g., an engineered nucleic acid construct or engineered nucleic acid-enzyme construct described herein) further comprises or encodes a DNA-repair modulating biomolecule, which may further enhance the efficiency of integration of a transgene on the heterologous nucleic acid by homology dependent repair (HDR).

In certain embodiments, the DNA-repair modulating biomolecule comprises a Nonhomologous end joining (NHEJ) inhibitor.

In certain embodiments, the DNA-repair modulating biomolecule comprises a homologous directed repair (HDR) promoter.

In certain embodiments, the DNA-repair modulating biomolecule comprises a NHEJ inhibitor and an HDR promoter.

In certain embodiments, the DNA-repair modulating biomolecule enhances or improves more precise genome editing and/or the efficiency of homologous recombination, compared to the otherwise identical embodiment without the DNA-repair modulating biomolecule.

HDR promoters and/or NHEJ inhibitors can, in some embodiments, comprise one or more small molecules. Systems bearing recombination enhancers such as small molecules that activate HDR and suppress NHEJ locally at the genomic site of the DNA damage can be tailored in their placement on the engineered systems to further enhance their efficiency. In general, the small molecule recombination enhancers can be synthesized to bear linkers and a functional group, such as maleimide for reacting with a thiol group on a Cys residue of a protein, for chemical conjugation to the engineered systems. Use of commercially available functionalized PEG linkers (alkyne, azide, cyclooctyne etc.) can also be employed for conjugation, and orthogonal conjugation chemistries can be utilized for the multivalent display.

Conjugation sites can be readily identified where modifications do not affect the potency of the recombination enhancers selected.

In certain embodiments, multivalent display of one or more DNA-repair modulating biomolecule can be affected, including multiple moieties of NHEJ inhibitors, HDR promoters, or a combination thereof. See, for example, “Genomic targeting of epigenetic probes using a chemically tailored Cas9 system” by Liszczak et al., Proc Natl Acad Sci U.S.A. 114:681-686, 2017 (incorporated herein by reference). In certain embodiments, multivalent display of small molecule compounds can be achieved through sortase loop proteins used as a scaffold for their display.

In some embodiments, the DNA-repair modulating biomolecule may comprise an HDR promoter. The HDR promoter may comprise small molecules, such as RSI or analogs thereof. In certain embodiments, the HDR promoter stimulates RAD51 activity or RAD52 motif protein 1 (RDM1) activity. In certain embodiments, the HDR promoter comprises Nocodazole, which can result in higher HDR selection.

In certain embodiments, the HDR promoter may be administered prior to the delivery of the engineered TnpB systems described herein.

In certain embodiments, the HDR promoter locally enhances HDR without NHEJ inhibition. For example, RAD51 is a protein involved in strand exchange and the search for homology regions during HDR repair. In certain embodiments, the HDR promoter is phenylbenzamide RSI, identified as a small-molecule RAD51-stimulator (see WO2019/135816 at [0200]-[0204], specifically incorporated herein by reference).

In certain embodiments, the DNA-repair modulating biomolecule comprises C-terminal binding protein interacting protein (CtIP) or a functional fragment or homolog thereof. CtIP is a key protein in early steps of homologous recombination. According to this embodiment, the CtIP or the functional fragment or homolog thereof can be linked (e.g., fused) to the RT or the sequence-specific nuclease (e.g., a CRISPR/Cas effector enzyme, a ZFN, a TALEN, a meganuclease, TnpB, IscB, or a restriction endonuclease (RE)), and stimulates transgene integration by HDR.

In certain embodiments, the CtIP fragment is a minimal N-terminal fragment of the wild-type CtIP, such as the N-terminal fragment comprising residues 1-296 of the full-length CtIP (the HE for HDR enhancer), as described in Charpentier et al. (Nature Comm., DOI: 10.1038/s41467-018-03475-7, incorporated herein by reference), shown to be sufficient to stimulate HDR. The activity of the fragment depends on CDK phosphorylation sites (e.g., S233, T245, and S276) and the multimerization domain essential for CtIP activity in homologous recombination. Thus alternative fragments comprising the CDK phosphorylation sites and the multimerization domain essential for CtIP activity are also within the scope of the invention.

In certain embodiments, the DNA-repair modulating biomolecule comprises a dominant negative 53BP1.

In certain embodiments, the DNA-repair modulating biomolecule comprises a cell cycle-specific degradation tag, such as the degradation domain of the (human) Geminin, and the (murine)CyclinB2.

In certain embodiments, the DNA-repair modulating biomolecule comprises CyclinB2, a member of the B-type cyclins that associate with p34cdc2, and an essential component of the cell cycle regulatory machinery. CRISPR-mediated knock-in efficiency may be increased by promoting the relative increase in Cas9 activity in G2 phase of the cell cycle, when HDR is more active. In certain embodiments, the degradation domains of the (human) Geminin and (murine)CyclinB2 can be used as either N- or C-terminal fusion to serve as the DNA-repair modulating biomolecule. These domains are known to determine a cell-cycle specific profile of chimeric proteins, namely an increase in their relative concentration in S and G2 compared to G1, high-jacking the conventional CyclinB2 and Geminin degradation pathways. This produces active Geminin-Cas9 and CyclinB2-Cas9 chimeric proteins, which are degraded in a cell-cycle-dependent manner. Such chimeras shift the repair of the DSBs to the HDR repair pathway compared to the commonly used Cas9.

While not wishing to be bound by particular theory, it is believed that the application of such cell cycle-specific degradation tags permits/promotes more efficient/secure gene editing.

In certain embodiments, the DNA-repair modulating biomolecule comprises a Rad family member protein, such as Rad50, Rad51, Rad52, etc., which functions to promote foreign DNA integration into a host chromosome. Specifically, Rad52 is an important homologous recombinant protein, and its complex with Rad51 plays a key role in HDR, mainly involved in the regulation of foreign DNA in eukaryotes. Key steps in the process of HR include repair mediated by Rad51 and strand exchange. Co-expression of Rad52 as a DNA-repair modulating biomolecule significantly enhances the likelihood of HDR by, e.g., three-fold.

In certain embodiments, the DNA-repair modulating biomolecule comprises a RAD52 protein as, e.g., either an N- or a C-terminal fusion.

In certain embodiments, the DNA-repair modulating biomolecule comprises a RAD52 motif protein 1 (RDM1) that functions similarly as RAD52. RDM1 has been shown to be able to repair DSBs caused by DNA replication, prevent G2 or M cell cycle arrest, and improve HDR selection.

In certain embodiments, the DNA-repair modulating biomolecule comprises a dominant negative version of the tumor suppressor p53-binding protein 1 (53BP1). The wild-type protein 53BP1 is a key regulator of the choice between NHEJ and HDR—it is a pro-NHEJ factor which limits HDR by blocking DNA end resection, and also by inhibiting BRCA1 recruitment to DSB sites. It has been shown that global inhibition of 53BP1 by a ubiquitin variant significantly improves Cas9-mediated HDR frequency in non-hematopoietic and hematopoietic cells with single-strand oligonucleotide delivery or double-strand donor in AAV.

In certain embodiments, the dominant negative (DN) version of the 53BP1 comprises the minimal focus forming region, but lacks domains outside this region, e.g., towards the N-terminus and tandem C-terminal BRCT repeats that recruit key effectors involved in NHEJ, such as RIFI-PTIP and EXPAND, respectively. The 53BP1 adapter protein is recruited to specific histone marks at sites of DSBs via this minimal focus forming region, which comprises several conserved domains including an oligomerization domain (OD), a glycine-arginine rich (GAR) motif, a Tudor domain, and an adjacent ubiquitin-dependent recruitment (UDR) motif. The Tudor domain mediates interactions with histone H4 dimethylated at K2023.

In certain embodiments, a dominant negative version of 53BP1 (DNIS) suppresses the accumulation of endogenous 53BP1 and downstream NHEJ proteins at sites of DNA damage, while upregulating the recruitment of the BRCA1 HDR protein. Such a DN version of the 53BP1 can be used as the DNA-repair modulating biomolecule, either as an N- or a C-terminal fusion (such as a Cas9 fusion, to locally inhibit NHEJ at the Cas9-target site defined by its gRNA, while promoting an increase in HDR, and does not globally affect NHEJ, thereby improving cell viability).

In certain embodiments, the DNA-repair modulating biomolecule comprises an NHEJ inhibitor, such as an inhibitor of DNA ligase IV, a KU inhibitor (e.g., KU70 or KU80), a DNA-PKc inhibitor, or an artemis inhibitor.

In certain embodiments, the NHEJ inhibitor inhibits the NHEJ pathway, enhances HDR, or modulates both. In certain embodiments, the NHEJ inhibitor is a small molecule inhibitor.

In certain embodiments, the small molecule inhibitor of the NHEJ pathway comprises an SCR7 analog, for example, PK66, PK76, PK409.

In certain embodiments, the NHEJ inhibitor comprises a KU inhibitor, for example, KU5788, and KU0060648.

In certain embodiments, a small molecule NHEJ inhibitor is linked to a polyglycine tripeptide through PEG for sortase-mediated ligation, as described in WO2019/135816, Guimaraes et al., Nat Protoc 8:1787-99, 2013; Theile et al., Nat Protoc 8:1800-7, 2013; and Schmohl et al., Curr Opin Chem Biol 22:122-8, 2014 (all incorporated herein by reference). The same means can also be used for attaching small molecule HDR enhancers to protein.

An exemplary method for conjugating a small molecule DNA-repair modulating biomolecule without loss of activity is described in WO2019135816, where SCR-7 conjugation of a poly-glycine peptide with the para-carboxylic moiety at ring 4 retained activity of the inhibitor, with rings 1, 2 and 3 of the molecule having involvement in the target-engagement, providing a simple and effective strategy to ligate a small molecule NHEJ inhibitor to the system described herein (e.g., to the sequence-specific nuclease including Cas enzymes, or to the RT) to precisely enhance HDR pathway near a nucleic acid target site.

In certain embodiments, a nucleic acid targeting moiety conjugates based on small molecule inhibitor of DNA-dependent protein kinase (DNA-PK) or heterodimeric Ku (KU70/KU80) can be utilized. KU-0060648 is one potent KU-inhibitors, which can also be functionalized with poly-glycine and used for recombination enhancement.

In certain embodiments, the DNA-repair modulating biomolecule comprises the Tumor Suppressor p53. p53 plays a direct role in DNA repair, including HR regulation, where it affects the extension of new DNA, thereby affecting HDR selection. In vivo, p53 binds to the nuclear matrix and is a rate-limiting factor in repairing DNA structure. p53 regulates DNA repair processes in almost all eukaryotes via transactivation-dependent and -independent pathways, but only the transactivation-independent function of p53 is involved in HR regulation. Wild-type p53 protein can link double stranded breaks to form intact DNA, as well as also playing a role in inhibiting NHEJ. p53 interacts with HR-related proteins, including Rad51, where it controls HR through direct interaction with Rad51.

### Inducibility and Self-Inactivation Modifications

In one embodiment, a TnpB polypeptide may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the TnpB polypeptide may be a part of a Light Inducible Transcriptional Effector (LITE) to direct changes in transcriptional activity in a sequence-specific manner. The components of a light may include a TnpB polypeptide, a light-responsive cytochrome heterodimer (e.g. from Arabidopsis thaliana), and a transcriptional activation/repression domain. Further examples of inducible DNA binding proteins and methods for their use are provided in U.S. Provisional Application Nos. 61/736,465 and U.S. 61/721,283, and International Patent Publication No. WO 2014/018423 A2 which is hereby incorporated by reference in its entirety.

Once all copies of a gene in the genome of a cell have been edited, continued expression of the system in that cell is no longer necessary. Indeed, sustained expression would be undesirable in case of off-target effects at unintended genomic sites, etc. Thus time-limited expression would be useful. Inducible expression offers one approach, but in addition Applicants have engineered a self-inactivating system that relies on the use of a non-coding nucleic acid component molecule target sequence within the vector itself. Thus, after expression begins, the system will lead to its own destruction, but before destruction is complete it will have time to edit the genomic copies of the target gene (which, with a normal point mutation in a diploid cell, requires at most two edits). Simply, the self-inactivating system includes additional RNA (e.g., nucleic acid component molecule) that targets the coding sequence for the TnpB polypeptide itself or that targets one or more non-coding nucleic acid component molecule target sequences complementary to unique sequences present in one or more of the following: (a) within the promoter driving expression of the non-coding RNA elements, (b) within the promoter driving expression of the TnpB polypeptide gene, (c) within 100 bp of the ATG translational start codon in the TnpB polypeptide coding sequence, (d) within the inverted terminal repeat (iTR) of a viral delivery vector, e.g., in the AAV genome.

In some aspects, a single nucleic acid component molecule is provided that is capable of hybridization to a sequence downstream of a TnpB polypeptide start codon, whereby after a period of time there is a loss of the TnpB polypeptide expression. In some aspects, one or more nucleic acid component molecule(s) are provided that are capable of hybridization to one or more coding or non-coding regions of the polynucleotide encoding the system, whereby after a period of time there is a inactivation of one or more, or in some cases all, of the system. In some aspects of the system, and not to be limited by theory, the cell may comprise a plurality of complexes, wherein a first subset of complexes comprise a first nucleic acid component molecule capable of targeting a genomic locus or loci to be edited, and a second subset of complexes comprise at least one second nucleic acid component molecule capable of targeting the polynucleotide encoding the system, wherein the first subset of complexes mediate editing of the targeted genomic locus or loci and the second subset of complexes eventually inactivate the system, thereby inactivating further expression in the cell.

The various coding sequences (TnpB polypeptide and nucleic acid component molecule) can be included on a single vector or on multiple vectors. For instance, it is possible to encode the enzyme on one vector and the various RNA sequences on another vector, or to encode the enzyme and one nucleic acid component molecule on one vector, and the remaining nucleic acid component molecule on another vector, or any other permutation. In general, a system using a total of one or two different vectors is preferred.

### C. Delivery Systems

The instant specification provides delivery systems for introducing components of the TnpB gene editing systems and compositions herein to cells, tissues, organs, or organisms. Depending on the chosen format, the TnpB gene editing systems and/or the individual or combined components thereof may be delivered as DNA molecules (e.g., encoded on one or more plasmids), RNA molecules (e.g., reRNAs for targeting the TnpB protein or linear or circular mRNAs coding for the TnpB protein or other protein components of the TnpB systems), proteins (e.g., TnpB polypeptides, accessory proteins having other functions (e.g., recombinases, nucleases, polymerases, ligases, deaminases, or reverse transcriptases), or protein-nucleic acid complexes (e.g., complexes between an reRNA and a TnpB protein or fusion protein comprising a TnpB protein).

The present disclosure contemplates any known method and/or technique for delivering the TnpB systems and compositions to cells, tissue, organs, or organisms. Delivery may involve in vitro, in vivo, or ex vivo methodologies.

A delivery system may comprise one or more delivery vehicles and/or cargos. Exemplary delivery systems and methods include those described in paragraphs to of Feng Zhang et al., (WO2016106236A1), and pages 1241-1251 and Table 1 of Lino C A et al., Delivering CRISPR: a review of the challenges and approaches, DRUGDELIVERY, 2018, VOL. 25, NO. 1, 1234-1257, which are incorporated by reference herein in their entireties and can be adapted for use with the TnpB proteins disclosed herein.

The example delivery compositions, systems, and methods described herein related to composition or TnpB polypeptide also apply to functional domains and other components (e.g., other proteins and polynucleotides related to the TnpB polypeptide, such as reverse transcriptase, nucleotide deaminase, retrotransposon, donor polynucleotide, etc.). In a preferred embodiment, the composition comprises delivery of the polypeptides via mRNA.

### 1. Vector Delivery Systems

Delivery of an engineered TnpB editing system to a cell can generally be accomplished with or without vectors.

The engineered TnpB editing system may be introduced into any type of cell, including any cell from a prokaryotic, eukaryotic, or archaeon organism, including bacteria, archaea, fungi, protists, plants (e.g., monocotyledonous and dicotyledonous plants); and animals (e.g., vertebrates and invertebrates). Examples of animals that may be transfected with an engineered TnpB editing system include, without limitation, vertebrates such as fish, birds, mammals (e.g., human and non-human primates, farm animals, pets, and laboratory animals), reptiles, and amphibians.

The engineered TnpB editing systems can be introduced into a single cell or a population of cells. Cells from tissues, organs, and biopsies, as well as recombinant cells, genetically modified cells, cells from cell lines cultured in vitro, and artificial cells (e.g., nanoparticles, liposomes, polymersomes, or microcapsules encapsulating nucleic acids) may all be used.

The engineered TnpB editing systems can be introduced into cellular fragments, cell components, or organelles (e.g., mitochondria in animal and plant cells, plastids (e.g., chloroplasts) in plant cells and algae).

Cells may be cultured or expanded after transfection with the engineered TnpB editing systems.

Methods of introducing nucleic acids into a host cell are well known in the art. Commonly used methods include chemically induced transformation, typically using divalent cations (e.g., CaCl₂)), dextran-mediated transfection, polybrene mediated transfection, lipofectamine and LT-1 mediated transfection, electroporation, protoplast fusion, encapsulation of nucleic acids in liposomes, and direct microinjection of the nucleic acids comprising engineered TnpB editing systems into nuclei. See, e.g., Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197; herein incorporated by reference in their entireties.

The engineered TnpB editing systems may also be used in plants. Methods for genetic transformation of plant cells are known in the art and include those set forth in US2022/0145296, and U.S. Pat. Nos. 8,575,425; 7,692,068; 8,802,934; 7,541,517; each of which is herein incorporated by reference in its entirety. See, also, Rakoczy-Trojanowska, M. (2002)Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G. W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; and Jones et al. (2005) Plant Methods 1:5.

The plant cells that have been transformed may be grown into a transgenic organism, such as a plant, in accordance with conventional methods. See, for example, McCormick et al. (1986) Plant Cell Reports 5:81-84.

Plant material that may be transformed with the engineered TnpB editing systems described herein includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Progeny, variants, and mutants of the regenerated plants are also included within the scope of the disclosure, provided that these parts comprise the genetic modification introduced by the engineered TnpB editing systems. Further provided is a processed plant product or byproduct that retains the genetic modification introduced by the engineered TnpB editing systems.

The engineered TnpB editing systems described herein may be used to produce transgenic plants with desired phenotypes, including but not limited to, increased disease resistance (e.g., increased viral, bacterial of fungal resistance), increased insect resistance, increased drought resistance, increased yield, and altered fruit ripening characteristics, sugar and oil composition, and color.

Vectors and/or nucleic acid molecules encoding the engineered TnpB editing systems or components thereof can include control elements.

Numerous vectors are available for use in the vector or vector system, including but not limited to, linear polynucleotides, polynucleotides associated with ionic or amphiphilic compounds, plasmids, and viruses.

Examples of viral vectors include, but are not limited to, adenoviral vectors, adeno-associated virus (AAV) vectors, retroviral vectors, lentiviral vectors, and the like. An expression construct can be replicated in a living cell, or it can be made synthetically.

In some embodiments, the nucleic acid comprising an engineered TnpB editing system is under transcriptional control of a promoter. In some embodiments, the promoter is competent for initiating transcription of an operably linked coding sequence by a RNA polymerase I, II, or III.

Exemplary promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression.

Exemplary promoters for plant cell expression include the CaMV 35S promoter (Odell et al., 1985, Nature 313:810-812); the rice actin promoter (McElroy et al., 1990, Plant Cell 2:163-171); the ubiquitin promoter (Christensen et al., 1989, Plant Mol. Biol. 12:619-632; and Christensen et al., 1992, Plant Mol. Biol. 18:675-689); the pEMU promoter (Last et al., 1991, Theor. Appl. Genet. 81:581-588); and the MAS promoter (Velten et al., 1984, EMBO J. 3:2723-2730).

In additional embodiments, the vectors for expressing and delivering the engineered TnpB editing systems may also comprise tissue-specific promoters to start expression only after it is delivered into a specific tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-b promoter, Mb promoter, Nphsl promoter, OG-2 promoter, SP-B promoter, SYN1 promoter, and WASP promoter.

These and other promoters can be obtained from or incorporated into commercially available plasmids, using techniques well known in the art. See, e.g., Sambrook et al., supra.

In some embodiments, one or more enhancer elements is/are used in association with the promoter to increase expression levels of the constructs. Examples include the SV40 early gene enhancer, as described in Dijkema et al., EMBOJ (1985) 4:761, the enhancer/promoter derived from the long terminal repeat (LTR) of the Rous Sarcoma Virus, as described in Gorman et al., Proc. Natl. Acad. Sci. USA (1982b) 79:6777, and elements derived from human CMV, as described in Boshart et al., Cell (1985) 41:521, such as elements included in the CMV intron A sequence. All such sequences are incorporated herein by reference.

In one embodiment, an expression vector comprises a promoter operably linked to a polynucleotide encoding the engineered TnpB editing system or component thereof.

In some embodiments, the vector or vector system also comprises a transcription terminator/polyadenylation signal. Examples of such sequences include, but are not limited to, those derived from SV40, as described in Sambrook et al., supra, as well as a bovine growth hormone terminator sequence (see, e.g., U.S. Pat. No. 5,122,458).

Additionally, 5′-UTR sequences can be placed adjacent to the coding sequence to further enhance the expression. Such sequences may include UTRs comprising an internal ribosome entry site (IRES). Inclusion of an IRES permits the translation of one or more open reading frames from a vector. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298: Rees et al., BioTechniques (1996) 20:102-110; Kobayashi et al., BioTechniques (1996) 21:399-402; and Mosser et al., BioTechniques (199722 ISO-161) c. A multitude of IRES sequences are known and include sequences derived from a wide variety of viruses, such as from leader sequences of picomaviruses such as the encephalomyocarditis virus (EMCV) UTR (Jang et al., Virol. (1989) 63:1651-1660). the polio leader sequence, the hepatitis A virus leader, the hepatitis C virus IRES, human rhinovirus type 2 IRES (Dobrikova et al., Proc. Natl. Acad. Sci. (2003) 100 (251:15125-151301)). an IRES element from the foot and mouth disease virus (Ramesh et al., Nucl. Acid Res. (1996) 24:2697-2700), a giardiavirus IRES (Garlapati et al., J Biol. Chem. (2004) 279 (51): 3389-33971) and the like. A variety of nonviral IRES sequences will also find use herein, including, but not limited to IRES sequences from yeast, as well as the human angiotensin II type 1 receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24 (17): 7622-7635), vascular endothelial growth factor IRES (Baranick et al. (2008) Proc. Natl. Acad Sci. U.S.A. 105 (12): 4733-4738, Stein et al. (1998) Mol. Cell. Biol. 18 (6): 3112-3119, Bert et al. (2006) RNA 12 (6): 1074-1083), and insulin-like growth factor 2 IRES (Pedersen et al. (2002) Biochem. J. 363 (Pt 1): 37-44).

These elements are commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org).

In some embodiments, a polynucleotide encoding a viral self-cleaving 2A peptide, such as a T2A peptide, can be used to allow production of multiple protein products (e.g., Cas9, bacteriophage recombination proteins, TnpBs) from a single vector or a single transcription unit under one promoter. One or more 2A linker peptides can be inserted between the coding sequences in the multicistronic construct. The 2A peptide, which is self-cleaving, allows co-expressed proteins from the multicistronic construct to be produced at equimolar levels. 2A peptides from various viruses may be used, including, but not limited to 2A peptides derived from the foot- and -mouth disease virus, equine rhinitis A virus, Jhosea asigna virus and porcine teschovirus-1. See, e.g., Kim et al. (2011) PLOS One 6 (4): el8556, Trichas et al. (2008) BMC Biol. 6:40, Provost et al. (2007) Genesis 45 (10): 625-629, Furler et al. (2001) Gene Ther. 8 (11): 864-873; herein incorporated by reference in their entireties.

In some embodiments, the expression construct comprises a plasmid suitable for transforming a bacterial host. Numerous bacterial expression vectors are known to those of skill in the art, and the selection of an appropriate vector is a matter of choice. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31 Bacterial plasmids may contain antibiotic selection markers (e.g., ampicillin, kanamycin, erythromycin, carbenicillin, streptomycin, or tetracycline resistance), a lacZ gene (b-galactosidase produces blue pigment from x-gal substrate), fluorescent markers (e.g., GFP. mCherry), or other markers for selection of transformed bacteria. See, e.g., Sambrook et al., supra.

In other embodiments, the expression construct comprises a plasmid suitable for transforming a yeast cell. Yeast expression plasmids typically contain a yeast-specific origin of replication (ORI) and nutritional selection markers (e.g., HIS3, URA3, LYS2, LEU2, TRP1, METIS, ura4+, leul+, ade6+), antibiotic selection markers (e.g., kanamycin resistance), fluorescent markers (e.g., mCherry), or other markers for selection of transformed yeast cells. The yeast plasmid may further contain components to allow shuttling between a bacterial host (e.g., E coif) and yeast cells. A number of different types of yeast plasmids are available including yeast integrating plasmids (Yip), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

In other embodiments, the expression construct comprises a virus or engineered construct derived from a viral genome. A number of viral based systems have been developed for gene transfer into mammalian cells. These include adenoviruses, retroviruses (g-retroviruses and lentiviruses), poxviruses, adeno-associated viruses, baculoviruses, and herpes simplex viruses (see e.g., Wamock et al. (2011) Methods Mol. Biol. 737:1-25; Walther et al. (2000) Drugs 60 (2): 249-271; and Lundstrom (2003) Trends Biotechnol. 21 (3): 117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Bums et al. (1993) Proc. Natl. Acad. Sci. USA 90:8033-8037; Boris-Lawrie and Temin (1993)Cur. Opin. Genet. Develop. 3:102-109; and Ferry et al. (2011)Curr. Pharm. Des. 17 (24): 2516-2527). Lentiviruses are a class of retroviruses that are particularly useful for delivering polynucleotides to mammalian cells because they are able to infect both dividing and nondividing cells (see e.g., Lois et al. (2002) Science 295:868-872; Durand et al. (2011) Viruses 3 (2): 132-159; herein incorporated by reference).

A number of adenoviral vectors have also been described. Unlike retroviruses which integrate into the host genome, adenoviruses persist extrachromosomally thus minimizing the risks associated with insertional mutagenesis.

Additionally, various adeno-associated vims (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990)(Cold Spring Harbor LaboratoryPress); Carter, B. J. Current Opinion in Biotechnology (1992) 3:533-539; Muzyczka, N. Current Topics in Microbiol and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering nucleic acids encoding the engineered TnpB editing systems is the enterically administered recombinant poxvirus vaccines described by Small, Jr., P. A., et al. (U.S. Pat. No. 5,676,950, issued Oct. 14, 1997, herein incorporated by reference).

Other viral vectors include those derived from the pox family of viruses, including vaccinia virus and avian poxvirus. By way of example, vaccinia virus recombinants expressing a nucleic acid molecule of interest (e.g., engineered TnpBs or recombinant TnpB ncRNAs) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

In some embodiments, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77:10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the nucleic acids of interest (e.g., engineered TnpB editing system) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the nucleic acid of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

In other approaches to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

Insect cell expression systems, such as baculovirus systems, can also be used and are known to those of skill in the art and described in, e.g., Baculovirus and Insect Cell Expression Protocols (Methods in Molecular Biology, D. W. Murhammer ed., Humana Press, 2nd edition, 2007) and L. King The Baculovirus Expression System: A laboratory guide (Springer, 1992). Materials and methods for baculovirus/insect cell expression systems are commercially available in kit form from, inter alia, Thermo Fisher Scientific (Waltham, MA) and Clontech (Mountain View, CA).

Plant expression systems can also be used for transforming plant cells. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

Several non-viral methods for the transfer of expression constructs into cultured cells also are contemplated. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau & Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

Once the expression construct has been delivered into the cell the nucleic acid comprising the engineered TnpB editing system may be positioned and expressed at different sites. In some embodiments, the nucleic acid comprising the engineered TnpB editing system may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or episomes encode sequences sufficient to permit maintenance and replication independent of or in synchronization with the host cell cycle. How the expression construct is delivered to a cell and where in the cell the nucleic acid remains is dependent on the type of expression construct employed.

In some embodiments, the expression construct may simply consist of naked recombinant DNA or plasmids comprising the engineered TnpB editing system. Transfer of the construct may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty & Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding an engineered TnpB editing system of interest may also be transferred in a similar manner in vivo and express the TnpB editing systems.

In still another embodiment, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987)Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

Other expression constructs which can be employed to deliver a nucleic acid into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167). Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87 (9): 3410-3414). A synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91 (9): 4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosy 1-ceramide, a galactose-terminal asialoganglioside, incorporated into liposomes and observed an increase in the uptake of the insulin gene by hepatocytes. Thus, it is feasible that a nucleic acid encoding a particular gene also may be specifically delivered into a cell by any number of receptor-ligand systems with or without liposomes. Also, antibodies to surface antigens on cells can similarly be used as targeting moieties.

In some embodiments, the promoters that may be used in the TnpB editing systems described herein may be constitutive, inducible, or tissue-specific. In some embodiments, the promoters may be a constitutive promoters. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EFla) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EFla promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-b promoter, Mb promoter, Nphsl promoter, OG-2 promoter, SP-B promoter, SYN1 promoter, and WASP promoter.

Also provided are vectors, including expression vectors, which comprise the above nucleic acid molecules of the present invention, as described further herein. In a first embodiment, the vectors include the isolated nucleic acid molecules described above. In an alternative embodiment, the vectors of the present invention include the above-described nucleic acid molecules operably linked to one or more expression control sequences. The vectors of the instant invention may thus be used to express one or more polypeptides.

Vectors useful for expression of nucleic acids are well known in the art. Exemplary vectors include one or more plasmids, a PCR amplicon or a viral vector suitable for delivery of TnpB genome editing system. In various embodiments, the viral vector is selected from a retroviral (retrovirus) vector, a lentiviral (lentivirus) vector, an adenoviral (adenovirus vector), an adeno-associated viral vector (adeno-associated viral (adeno) vector), associated virus (AAV) vector), vaccinia viral (vaccinia virus) vector, poxviral (poxvirus) vector, and herpes simplex viral (herpes simplex virus) vector).

### 2. Non-Viral Delivery Systems

The engineered TnpB editing systems can be delivered by any known non-viral delivery system. Non-limiting examples of delivery vehicles include lipid particles (e.g. Lipid nanoparticles (LNPs)), non-lipid nanoparticles, exosomes, liposomes, micelles, viral particles, Stable nucleic-acid-lipid particles (SNALPs), lipoplexes/polyplexes, Gold nanoparticles, iTOP, Streptolysin O (SLO), multifunctional envelope-type nanodevice (MEND), lipid-coated mesoporous silica particles, inorganic nanoparticles, and polymeric delivery technology (e.g., polymer-based particles).

### Liposomes

In a further embodiment, expression construct encoding the engineered TnpB editing systems may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In some embodiments, the liposome may be complexed with a hemagglutinating virus (HVJ). This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I)(Kato et al. (1991) J. Biol. Chem. 266 (6): 3361-3364).

In yet further embodiments, the liposome may be complexed or employed in conjunction with both HVJ and HMG-I. Where a bacterial promoter is employed in the DNA construct, it also will be desirable to include within the liposome an appropriate bacterial polymerase.

In one embodiment, a lipid particle may be liposome. Liposomes are spherical vesicle structures composed of a uni- or multilamellar lipid bilayer surrounding internal aqueous compartments and a relatively impermeable outer lipophilic phospholipid bilayer. In one embodiment, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In one embodiment, the liposome comprises a transport polymer, which may optionally be branched, comprising at least 10 amino acids and a ratio of histidine to non-histidine amino acids greater than 1.5 and less than 10. The branched transport polymer can comprise one or more backbones, one or more terminal branches, and optionally, one or more non-terminal branches. See, U.S. Pat. No. 7,070,807, incorporated herein by reference in its entirety. In one embodiment, the transposrt polymer is a Histidine-Lysine co-polymer (HKP) used to package and deliver mRNA and other cargos. See, U.S. Pat. Nos. 7,163,695, and 7,772,201, incorporated herein by reference in their entireties,

In one embodiment, the lipid particles may be stable nucleic acid lipid particles (SNALPs). SNALPs may comprise an ionizable lipid (DLinDMA)(e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol) 2000) carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-eDMA, and 1,2-dilinoleyloxy-3-(N;N-dimethyl) aminopropane (DLinDMA)

### Polymer Based Vehicles

In one embodiment, the delivery vehicles may comprise polymer-based particles (e.g., nanoparticles). In one embodiment, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or snucleic acid component, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In one embodiment, the polymer-based particles may comprise alkylated and carboxyalkylated branched polyethylenimine. In some examples, the polymer-based particles are VIROMER, e.g., VIROMER RNAi, VIROMER RED, VIROMER mRNA. Example methods of delivering the systems and compositions herein include those described in Bawage S S et al., Synthetic mRNA expressed Cast 3a mitigates RNA virus infections, biorxiv.org/content/10.1101/370460vl. full doi: doi.org/10.1101/370460, Viromer® RED, a powerful tool for transfection of keratinocytes. doi: 10.13140/RG.2.2.16993.61281, Viromer® Transfection-Factbook 2018: technology, product overview, users' data., doi: 10.13140/RG.2.2.23912.16642.

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January;267 (1): 9-21; El-Andaloussi S, et al., Nat Protoc. 2012 December;7 (12): 2112-26; Uno Y, et al., Hum

Gene Ther. 2011 June;22 (6): 711-9; Zou W, et al., Hum Gene Then 2011 April;22 (4): 465-75. Exemplary exosomes can be generated from 293F cells, with mRNA-loaded exosomes driving higher mRNA expression than mRNA loaded LNPs in some instances. See, e.g. J. Biol. Chem. (2021) 297 (5) 101266

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

### Lipid Nanoparticles (LNP)

The payloads (e.g., linear and circular mRNAs; nucleobase editing systems and/or components thereof) described herein may be encapsulated and delivered by lipid nanoparticles (LNPs) and compositions and/or formulations comprising RNA-encapsulated LNPs.

Below describes LNPs that may be used as the payload delivery vehicles contemplated herein, as well as the various ionizable lipids, structural lipids, PEGylated lipids, and phospholipids that may be used to make the herein LNPs for delivery payloads to cells. In addition, below describes additional LNP components that are contemplated, such as targeting moieties and other lipid components.

### Lipid Nanoparticle Compositions

In one aspect, the present disclosure further provides delivery systems for delivery of a therapeutic payload (e.g., the RNA payloads described herein which may encode a polypeptide of interest, e.g., a nucleobase editing system or a therapeutic protein) disclosed herein. In some embodiments, a delivery system suitable for delivery of the therapeutic payload disclosed herein comprises a lipid nanoparticle (LNP) formulation.

In some embodiments, an LNP of the present disclosure comprises an ionizable lipid, a structural lipid, a PEGylated lipid (aka PEG lipid), and a phospholipid. In alternative embodiments, an LNP comprises an ionizable lipid, a structural lipid, a PEGylated lipid (aka PEG lipid), and a zwitterionic amino acid lipid. In some embodiments, an LNP further comprises a 5th lipid, besides any of the aforementioned lipid components. In some embodiments, the LNP encapsulates one or more elements of the active agent of the present disclosure. In some embodiments, an LNP further comprises a targeting moiety covalently or non-covalently bound to the outer surface of the LNP. In some embodiments, the targeting moiety is a targeting moiety that binds to, or otherwise facilitates uptake by, cells of a particular organ system.

In some embodiments, an LNP has a diameter of at least about 20 nm, 30 nm, 40 nm, 50 nm, 60 nm, 70 nm, 80 nm, or 90 nm. In some embodiments, an LNP has a diameter of less than about 100 nm, 110 nm, 120 nm, 130 nm, 140 nm, 150 nm, or 160 nm. In some embodiments, an LNP has a diameter of less than about 100 nm. In some embodiments, an LNP has a diameter of less than about 90 nm. In some embodiments, an LNP has a diameter of less than about 80 nm. In some embodiments, an LNP has a diameter of about 60-100 nm. In some embodiments, an LNP has a diameter of about 75-80 nm.

In some embodiments, the lipid nanoparticle compositions of the present disclosure are described according to the respective molar ratios of the component lipids in the formulation. As a non-limiting example, the mol-% of the ionizable lipid may be from about 10 mol-% to about 80 mol-%. As a non-limiting example, the mol-% of the ionizable lipid may be from about 20 mol-% to about 70 mol-%. As a non-limiting example, the mol-% of the ionizable lipid may be from about 30 mol-% to about 60 mol-%. As a non-limiting example, the mol-% of the ionizable lipid may be from about 35 mol-% to about 55 mol-%. As a non-limiting example, the mol-% of the ionizable lipid may be from about 40 mol-% to about 50 mol-%.

In some embodiments, the mol-% of the phospholipid may be from about 1 mol-% to about 50 mol-%. In some embodiments, the mol-% of the phospholipid may be from about 2 mol-% to about 45 mol-%. In some embodiments, the mol-% of the phospholipid may be from about 3 mol-% to about 40 mol-%. In some embodiments, the mol-% of the phospholipid may be from about 4 mol-% to about 35 mol-%. In some embodiments, the mol-% of the phospholipid may be from about 5 mol-% to about 30 mol-%. In some embodiments, the mol-% of the phospholipid may be from about 10 mol-% to about 20 mol-%. In some embodiments, the mol-% of the phospholipid may be from about 5 mol-% to about 20 mol-%.

In some embodiments, the mol-% of the structural lipid may be from about 10 mol-% to about 80 mol-%. In some embodiments, the mol-% of the structural lipid may be from about 20 mol-% to about 70 mol-%. In some embodiments, the mol-% of the structural lipid may be from about 30 mol-% to about 60 mol-%. In some embodiments, the mol-% of the structural lipid may be from about 35 mol-% to about 55 mol-%. In some embodiments, the mol-% of the structural lipid may be from about 40 mol-% to about 50 mol-%.

In some embodiments, the mol-% of the PEG lipid may be from about 0.1 mol-% to about 10 mol-%. In some embodiments, the mol-% of the PEG lipid may be from about 0.2 mol-% to about 5 mol-%. In some embodiments, the mol-% of the PEG lipid may be from about 0.5 mol-% to about 3 mol-%. In some embodiments, the mol-% of the PEG lipid may be from about 1 mol-% to about 2 mol-%. In some embodiments, the mol-% of the PEG lipid may be about 1.5 mol-%. In some embodiments, the mol-% of the PEG lipid may be about 2.5 mol-%.

i. Ionizable Lipids

In some embodiments, an LNP disclosed herein comprises an ionizable lipid. In some embodiments, an LNP comprises two or more ionizable lipids.

Described below are a number of exemplary ionizable lipids of the present disclosure.

In some embodiments, an LNP of the present disclosure comprises an ionizable lipid disclosed in one of US 2023/0053437; US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, an LNP described herein comprises a lipid, e.g., an ionizable lipid, disclosed in US Application publication US2017/0119904, which is incorporated by reference herein, in its entirety.

In some embodiments, an LNP described herein comprises a lipid, e.g., an ionizable lipid, disclosed in PCT Application publication WO2021/204179, which is incorporated by reference herein, in its entirety.

In some embodiments, an LNP described herein comprises a lipid, e.g., an ionizable lipid, disclosed in PCT Application WO2022/251665A1, which is incorporated by reference herein, in its entirety.

In some embodiments, an LNP described herein comprises an ionizable lipid of Table Z:

TABLE Z
Exemplary Ionizable Lipids

| Compound # | Structure |
|---|---|
| L-1 | 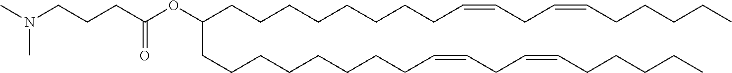 [SMILES: CCCCC/C=C\C/C=C\CCCCCCCCC(CCCCCCCC/C=C\C/C=C\CCCCC)OC(=O)CCCN(C)C] |
| L-2 | 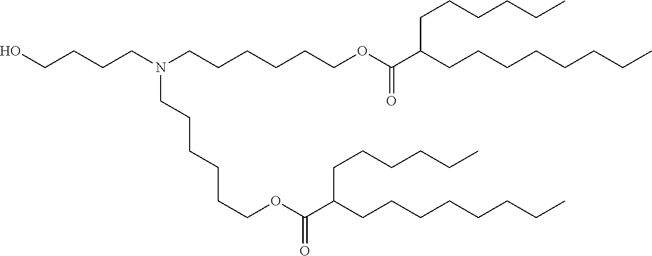 [SMILES: CCCCCCCCC(CCCCCC)C(=O)OCCCCCCN(CCCCO)CCCCCCOC(=O)C(CCCCCC)CCCCCCCC] |
| L-3 | 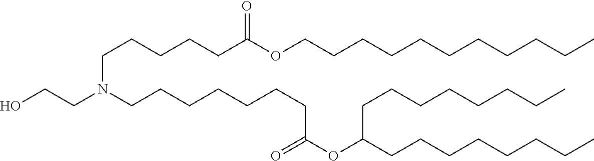 [SMILES: CCCCCCCCCCCOC(=O)CCCCCN(CCO)CCCCCCCC(=O)OC(CCCCCCCC)CCCCCCCC] |
| L-4 | 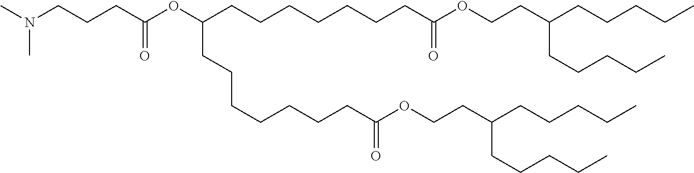 [SMILES: CCCCCC(CCCCC)CCOC(=O)CCCCCCCC(CCCCCCCC(=O)OCCC(CCCCC)CCCCC)OC(=O)CCCN(C)C] |
| L-5 | 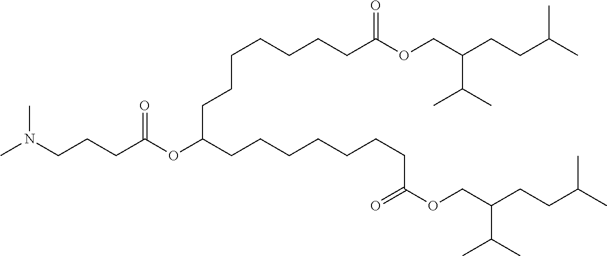 [SMILES: CC(C)CCC(COC(=O)CCCCCCCC(CCCCCCCC(=O)OCC(CCC(C)C)C(C)C)OC(=O)CCCN(C)C)C(C)C] |
| L-6 | 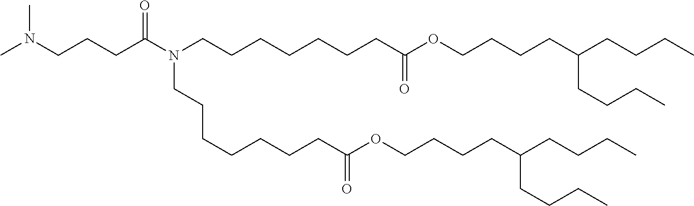 [SMILES: CCCCC(CCCC)CCCCOC(=O)CCCCCCCN(CCCCCCCC(=O)OCCCCC(CCCC)CCCC)C(=O)CCCN(C)C] |
| L-7 | 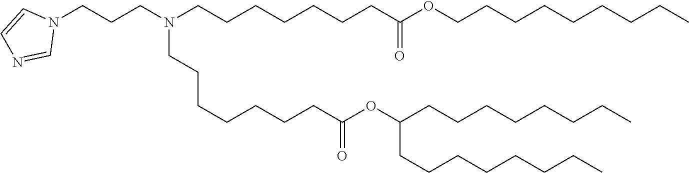 [SMILES: CCCCCCCCCOC(=O)CCCCCCCN(CCCCCCCC(=O)OC(CCCCCCCC)CCCCCCCC)CCCn1ccnc1] |
| L-8 | 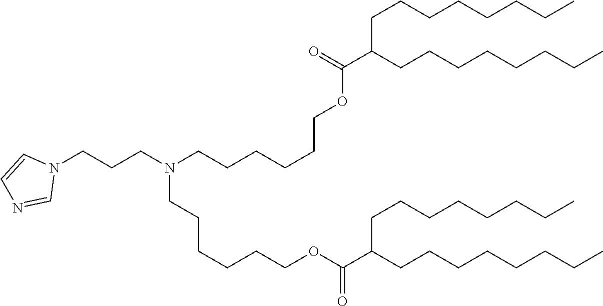 [SMILES: CCCCCCCCC(CCCCCCCC)C(=O)OCCCCCCN(CCCCCCOC(=O)C(CCCCCCCC)CCCCCCCC)CCCn1ccnc1] |
| L-9 | 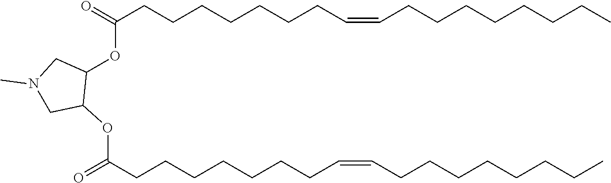 [SMILES: CCCCCCCC/C=C\CCCCCCCC(=O)OC1CN(C)CC1OC(=O)CCCCCCC/C=C\CCCCCCCC] |
| L-10 | 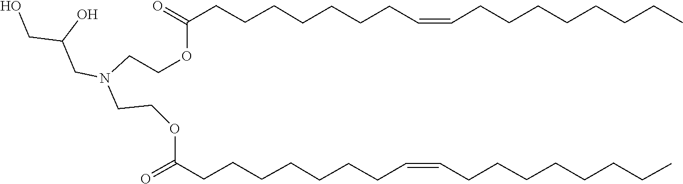 [SMILES: CCCCCCCC/C=C\CCCCCCCC(=O)OCCN(CCOC(=O)CCCCCCC/C=C\CCCCCCCC)CC(O)CO] |

In some embodiments, the ionizable lipid is MC3.

In some embodiments, an LNP of the present disclosure comprises an ionizable lipid disclosed in PCT Application Publication WO2023044343A1, which is incorporated by reference herein, in its entirety.

### Formula (VII-A)

In some embodiments, Lipids of the Disclosure have a structure of Formula (VII-A):

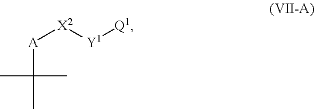

or a pharmaceutically acceptable salt thereof, wherein:
A is —N(—X¹R¹)—, —C(R′)(-L¹-N(R″)R⁶)—, —C(R′)(—OR^{7a})—, —C(R′)(—N(R″)R^{8a})—, —C(R′)(—C(═O)OR^{9a})—, —C(R′)(—C(═O)N(R″)R^{10a})—, or —C(═N—R^{11a})—;
T is —X^{2a}—Y^{1a}-Q^{1a} or —X³—C(═O)OR⁴;
X¹ is optionally substituted C₂-C₆ alkylenyl;
R¹ is —OH, —R^{1a},

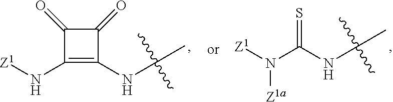

Z¹ is optionally substituted C₁-C₆ alkyl;
Z^{1a} is hydrogen or optionally substituted C₁-C₆ alkyl;
X² and X^{2a} are independently optionally substituted C₂-C₁₄ alkylenyl or optionally substituted C₂-C₁₄ alkenylenyl;
X³ is optionally substituted C₂-C₁₄ alkylenyl or optionally substituted C₂-C₁₄ alkenylenyl;
(i) Y1 is

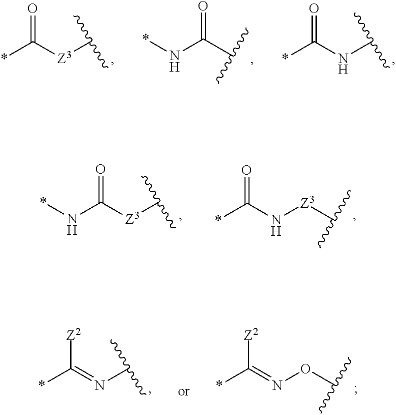

wherein the bond marked with an “*” is attached to X²;
Y^{1a} is

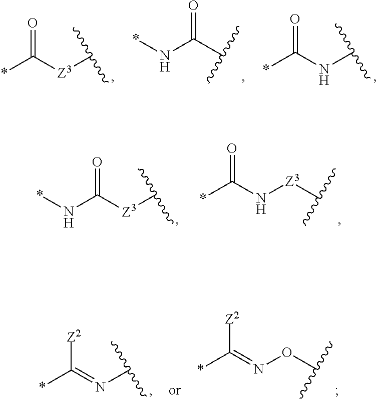

wherein the bond marked with an “*” is attached to X^{2a}.
each Z² is independently H or optionally substituted C₁-C₈ alkyl;
each Z³ is independently optionally substituted C₁-C₆ alkylenyl;
Q¹ is —NR²R³, —CH(OR²)(OR³), —CR²═C(R³)(R¹²), or —C(R²)(R³)(R¹²);
Q^{1a} is —NR²′R³′, —CH(OR²′)(OR³′), —CR²═C(R³)(R¹²), or —C(R²′)(R³′)(R¹²′); or

### (ii) Y¹ is

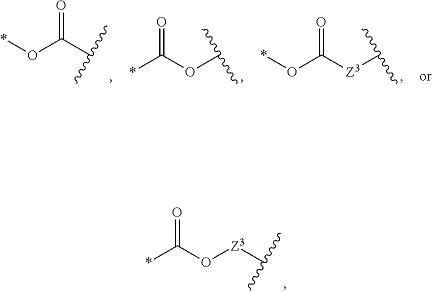

wherein the bond marked with an “*” is attached to X²;
Y^{1a} is

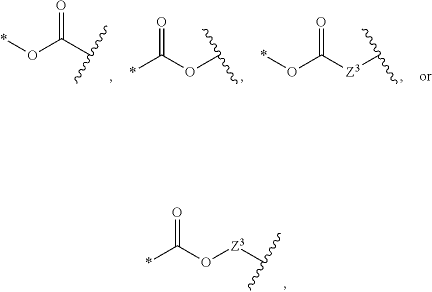

wherein the bond marked with an “*” is attached to X²a.
each Z² is independently H or optionally substituted C₁-C₈ alkyl;
each Z³ is independently optionally substituted C₁-C₆ alkylenyl;
Q¹ is —NR²R³;
Q^{1a} is —NR²′R³′;
R², R³, and R¹² are independently hydrogen, optionally substituted C₁-C₁₄ alkyl, optionally substituted C₂-C₁₄ alkenylenyl, or —(CH₂)ₘ-G-(CH₂)ₙH;
R²′, R³′, and R¹²′ are independently hydrogen, optionally substituted C₁-C₁₄ alkyl, optionally substituted C₂-C₁₄ alkenylenyl, or —(CH₂)ₘ-G-(CH₂)ₙH;
G is a C₃-C₈ cycloalkylenyl;
each m is independently 0, 1, 2, 3, 4, 5, 6, 7, 8, 9, 10, 11, or 12;
each n is independently 0, 1, 2, 3, 4, 5, 6, 7, 8, 9, 10, 11, or 12;
X³ is optionally substituted C₂-C₁₄ alkylenyl;
R⁴ is optionally substituted C₄-C₁₄ alkyl;
L¹ is C₁-C₈ alkylenyl;
R⁶ is C₁-C₆ alkyl, (hydroxy)C₁-C₆ alkyl, or (amino)C₁-C₆ alkyl
R^{7a} is —C(═O)N(R″)R^{7b}, —C(═S)N(R″)R^{7b}, —N═C(R^{7b})(R^{7c}), or

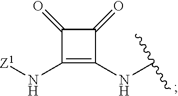

R^{7b} is C₁-C₆ alkyl, (hydroxy)C₁-C₆ alkyl, or (amino)C₁-C₆ alkyl;
R^{7c} is hydrogen or C₁-C₆ alkyl;
R^{8a} is —C(═O)N(R′″)R^{8b}, —C(═S)N(R′″)R^{8b}, —N═C(R^{8b})(R^{8c}), or

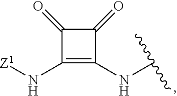

R^{8b} is C₁-C₆ alkyl, (hydroxy)C₁-C₆ alkyl, or (amino)C₁-C₆ alkyl;
R^{8c} is hydrogen or C₁-C₆ alkyl;
R^{9a} is —N═C(R^{9b})(R^{9c});
R^{9b} is C₁-C₆ alkyl, (hydroxy)C₁-C₆ alkyl, or (amino)C₁-C₆ alkyl;
R^{9c} is hydrogen or C₁-C₆ alkyl;
R^{10a} is —N═C(R^{10b})(R^{10c});
R^{10b} is C₁-C₆ alkyl, (hydroxy)C₁-C₆ alkyl, or (amino)C₁-C₆ alkyl;
R^{10c} is hydrogen or C₁-C₆ alkyl;
R^{11a} is —OR^{11b}, —N(R″)R^{11b}, —OC(═O)R^{11b}, or —N(R″)C(═O)R^{11b},
R^{11b} is C₁-C₆ alkyl, (hydroxy)C₁-C₆ alkyl, or (amino)C₁-C₆ alkyl;
R′ is hydrogen or C₁-C₆ alkyl;
R″ is hydrogen or C₁-C₆ alkyl; and
R′″ is hydrogen or C₁-C₆ alkyl.

### Formula (VIII-A)

In some embodiments, Lipids of the Disclosure have a structure of Formula (VII-A), wherein the Lipids of the Disclosure have a structure of Formula (VIII-A):

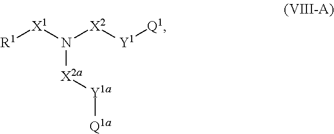

or a pharmaceutically acceptable salt thereof.

### Formula (VII-B)

In some embodiments, Lipids of the Disclosure have a structure of Formula (VII-B):

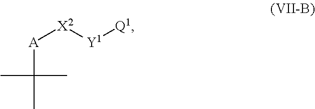

or a pharmaceutically acceptable salt thereof, wherein:
A is —C(R′)(-L¹-N(R″)R⁶)—, —C(R′)(—OR^{7a})—, —C(R′)(—N(R″)R^{8a})—, —C(R′)(—C(═O)OR^{9a})—, —C(R′)(—C(═O)N(R″)R^{10a})—, or —C(═N—R^{11a})—;
T is —X^{2a}—Y^{1a}-Q^{1a} or —X³—C(═O)OR⁴;
X² and X^{2a} are independently optionally substituted C₂-C₁₄ alkylenyl or optionally substituted C₂-C₁₄ alkenylenyl;
X³ is optionally substituted C₁-C₁₄ alkylenyl or optionally substituted C₂-C₁₄ alkenylenyl;
Y¹ is

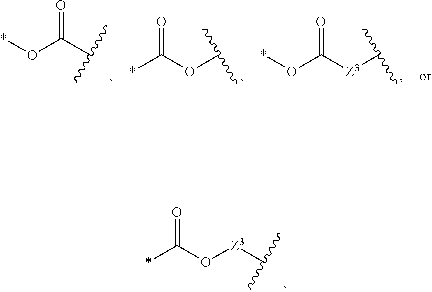

wherein the bond marked with an “*” is attached to X²;
Y^{1a} is

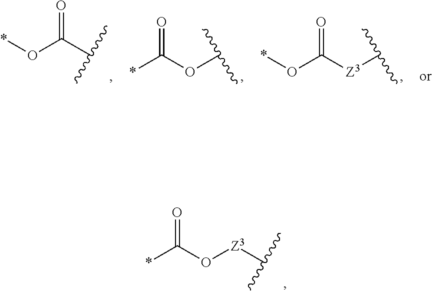

wherein the bond marked with an “*” is attached to X²a,
each Z³ is independently optionally substituted C₁-C₆ alkylenyl or optionally substituted C₂-C₁₄ alkenylenyl;
Q¹ is —NR²R³, —CH(OR²)(OR³), —CR²═C(R³)(R¹²), or —C(R²)(R³)(R¹²);
Q^{1a} is —NR²′R³′, —CH(OR²′)(OR³′), —CR²═C(R³)(R¹²), or —C(R²′)(R³′)(R¹²′);
R², R³, and R¹² are independently hydrogen, optionally substituted C₁-C₁₄ alkyl, optionally substituted C₂-C₁₄ alkenylenyl, or —(CH₂)ₘ-G-(CH₂)ₙH;
R²′, R³′, and R¹²′ are independently hydrogen, optionally substituted C₁-C₁₄ alkyl, optionally substituted C₂-C₁₄ alkenylenyl, or —(CH₂)ₘ-G-(CH₂)ₙH; G is a C₃-C₈ cycloalkylenyl;
each m is independently 0, 1, 2, 3, 4, 5, 6, 7, 8, 9, 10, 11, or 12;
each n is independently 0, 1, 2, 3, 4, 5, 6, 7, 8, 9, 10, 11, or 12;
X³ is optionally substituted C₂-C₁₄ alkylenyl;
R⁴ is optionally substituted C₄-C₁₄ alkyl;
L¹ is C₁-C₈ alkylenyl;
R⁶ is (hydroxy)C₁-C₆ alkyl, or (amino)C₁-C₆ alkyl.
R^{7a} is —C(═O)N(R′″)R^{7b}, —C(═S)N(R′″)R^{7b}, —N═C(R^{7b})(R^{7c}),

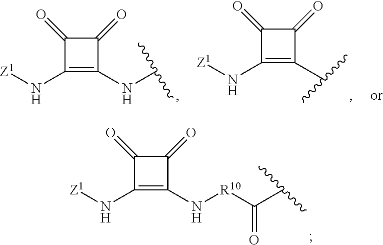

Z¹ is optionally substituted C₁-C₆ alkyl;
R¹⁰ is C₁-C₆ alkylenyl;
R^{7b} is C₁-C₆ alkyl, (hydroxy)C₁-C₆ alkyl, or (amino)C₁-C₆ alkyl;
R^{7c} is hydrogen or C₁-C₆ alkyl;
R^{8a} is —C(═O)N(R′″)R^{8b}, —C(═S)N(R′″)R^{8b}, —N═C(R^{8b})(R^{8C}),

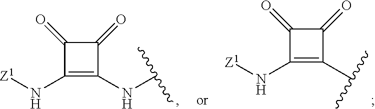

R^{8b} is C₁-C₆ alkyl, (hydroxy)C₁-C₆ alkyl, or (amino)C₁-C₆ alkyl;
R^{8c} is hydrogen or C₁-C₆ alkyl;
R^{9a} is —N═C(R⁹%)(R^{8c});
R^{9b} is C₁-C₆ alkyl, (hydroxy)C₁-C₆ alkyl, or (amino)C₁-C₆ alkyl;
R^{9c} is hydrogen or C₁-C₆ alkyl;
R^{10a} is —N═C(R^{10b})(R^{10c});
R^{10b} is C₁-C₆ alkyl, (hydroxy)C₁-C₆ alkyl, or (amino)C₁-C₆ alkyl;
R^{10c} is hydrogen or C₁-C₆ alkyl;
R^{11a} is —OR^{11b}, —N(R″)R^{11b}, —OC(═O)R^{11b}, or —N(R″)C(═O)R^{11b};
R^{11b} is C₁-C₆ alkyl, (hydroxy)C₁-C₆ alkyl, or (amino)C₁-C₆ alkyl;
R′ is hydrogen or C₁-C₆ alkyl;
R″ is hydrogen or C₁-C₆ alkyl; and
R′″ is hydrogen or C₁-C₆ alkyl.

In some embodiments, Lipids of the Disclosure have a structure of Formula (VII-B), wherein A is —C(R′)(-L¹-N(R″)R⁶)—.

In some embodiments, Lipids of the Disclosure have a structure of Formula (VII-B), wherein A is —C(R′)(—OR^{7a})—.

In some embodiments, Lipids of the Disclosure have a structure of Formula (VII-B), wherein A is —C(R′)(—N(R″)R^{8a}).

In some embodiments, Lipids of the Disclosure have a structure of Formula (VII-B), wherein A is —C(R′)(—C(═O)OR^{9a}).

In some embodiments, Lipids of the Disclosure have a structure of Formula (VII-B), wherein A is —C(R′)(—C(═O)N(R″)R^{10a})—.

In some embodiments, Lipids of the Disclosure have a structure of Formula (VII-B), wherein A is —C(═N—R^{11a})—.

In some embodiments, Lipids of the Disclosure have a structure of Formula (VII-B), wherein T is —X^{2a}—Y^{1a}-Q^{1a}

In some embodiments, Lipids of the Disclosure have a structure of Formula (VII-B), wherein T is —X³—C(═O)OR⁴.

In some embodiments, Lipids of the Disclosure have a structure of Formula (VII-B), wherein X² and/or X^{2a} are/is optionally substituted C₂-C₁₄ alkylenyl (e.g., C₂-C₁₀ alkylenyl, C₂-C₈ alkylenyl, C₂, C₃, C₄, C₅, C₆, C₇, or C₈ alkylenyl). In some embodiments, Lipids of the Disclosure have a structure of Formula (VII-B), wherein X² is C₂-C₁₄ alkylenyl. In some embodiments, Lipids of the Disclosure have a structure of Formula (VII-B), wherein X^{2a} is C₂-C₁₄ alkylenyl

In some embodiments, Lipids of the Disclosure have a structure of Formula (VII-B), wherein Y¹ and/or Y^{1a} are/is

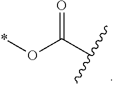 [SMILES: *OC(*)=O]

In some embodiments, Lipids of the Disclosure have a structure of Formula (VII-B), wherein Y¹ is

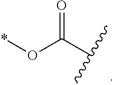 [SMILES: *OC(*)=O]

In some embodiments, Lipids of the Disclosure have a structure of Formula (VII-B), wherein Y^{1a} is

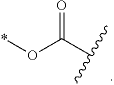 [SMILES: *OC(*)=O]

In some embodiments, Lipids of the Disclosure have a structure of Formula (VII-B), wherein Y¹ and/or Y¹a are/is

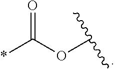 [SMILES: *OC(*)=O]

In some embodiments, Lipids of the Disclosure have a structure of Formula (VII-B), wherein Y¹ is

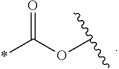 [SMILES: *OC(*)=O]

In some embodiments, Lipids of the Disclosure have a structure of Formula (VII-B), wherein Y^{1a} is

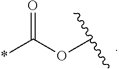 [SMILES: *OC(*)=O]

In some embodiments, Lipids of the Disclosure have a structure of Formula (VII-B), wherein Y¹ and/or Y^{1a} are/is

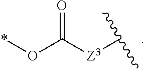

In some embodiments, Lipids of the Disclosure have a structure of Formula (VII-B), wherein Y¹ is

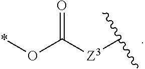

In some embodiments, Lipids of the Disclosure have a structure of Formula (VII-B), wherein Y^{1a} is

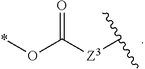

In some embodiments, Lipids of the Disclosure have a structure of Formula (VII-B), wherein Y¹ and/or Y^{1a} are/is

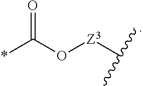

In some embodiments, Lipids of the Disclosure have a structure of Formula (VII-B), wherein Y¹ is

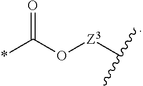

In some embodiments, Lipids of the Disclosure have a structure of Formula (VII-B), wherein Y^{1a} is

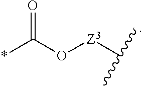

In some embodiments, Lipids of the Disclosure have a structure of Formula (VII-B), wherein Q¹ and/or Q^{1a}are/is —C(R²′)(R³′)(R¹²′). In some embodiments, Lipids of the Disclosure have a structure of Formula (VII-B), wherein Q¹ is —C(R²′)(R³′)(R¹²′). In some embodiments, Lipids of the Disclosure have a structure of Formula (VII-B), wherein Q^{1a} is —C(R²′)(R³′)(R¹²′).

In some embodiments, Lipids of the Disclosure have a structure of Formula (VII-B), wherein X³ is optionally substituted C₁-C₁₄ alkylenyl (e.g., C₁-C₆, C₁-C₄ alkylenyl). In some embodiments, Lipids of the Disclosure have a structure of Formula (VII-B), wherein X³ is C₁-C₁₄ alkylenyl.

In some embodiments, Lipids of the Disclosure have a structure of Formula (VII-B), wherein R², R³, R¹², R²′, R³′, and/or R¹²′ are hydrogen. In some embodiments, Lipids of the Disclosure have a structure of Formula (VII-B), wherein R² is hydrogen. In some embodiments, Lipids of the Disclosure have a structure of Formula (VII-B), wherein R³ is hydrogen. In some embodiments, Lipids of the Disclosure have a structure of Formula (VII-B), wherein R¹² is hydrogen. In some embodiments, Lipids of the Disclosure have a structure of Formula (VII-B), wherein R²′ is hydrogen. In some embodiments, Lipids of the Disclosure have a structure of Formula (VII-B), wherein R³′ is hydrogen. In some embodiments, Lipids of the Disclosure have a structure of Formula (VII-B), wherein R¹²′ is hydrogen.

In some embodiments, Lipids of the Disclosure have a structure of Formula (VII-B), wherein R², R³, R¹², R²′, R³′, and/or R¹²′ are optionally substituted C₁-C₁₄ alkyl (e.g., C₄-C₁₀ alkyl, C₅, C₆. C₇. C₈, C₉ alkyl). In some embodiments, Lipids of the Disclosure have a structure of Formula (VII-B), wherein R² is C₄-C₁₀ alkyl. In some embodiments, Lipids of the Disclosure have a structure of Formula (VII-B), wherein R³ is C₄-C₁₀ alkyl. In some embodiments, Lipids of the Disclosure have a structure of Formula (VII-B), wherein R¹² is C₄-C₁₀ alkyl. In some embodiments, Lipids of the Disclosure have a structure of Formula (VII-B), wherein R²′ is C₄-C₁₀ alkyl. In some embodiments, Lipids of the Disclosure have a structure of Formula (VII-B), wherein R³′ is C₄-C₁₀ alkyl. In some embodiments, Lipids of the Disclosure have a structure of Formula (VII-B), wherein R¹²′ is C₄-C₁₀ alkyl.

In some embodiments, Lipids of the Disclosure have a structure of Formula (VII-B), wherein R⁴ is optionally substituted C₄-C₁₄ alkyl (e.g., C₈-C₁₄ alkyl, linear C₈-C₁₄ alkyl, C₈, C₉, C₁₀, C₁₁, C₁₂, C₁₃, or C₁₄ alkyl). In some embodiments, Lipids of the Disclosure have a structure of Formula (VII-B), wherein R⁴ is linear C₈-C₁₄ alkyl. In some embodiments, Lipids of the Disclosure have a structure of Formula (VII-B), wherein R⁴ is linear C₁₁ alkyl.

In some embodiments, Lipids of the Disclosure have a structure of Formula (VII-B), wherein L¹ is C₁-C₃ alkylenyl.

In some embodiments, Lipids of the Disclosure have a structure of Formula (VII-B), wherein R⁶ is (hydroxy)C₁-C₆ alkyl.

In some embodiments, Lipids of the Disclosure have a structure of Formula (VII-B), wherein R^{7a} is

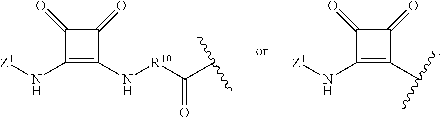

In some embodiments, Lipids of the Disclosure have a structure of Formula (VII-B), wherein R^{7a} is

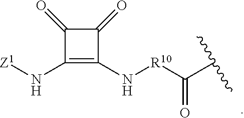

In some embodiments, Lipids of the Disclosure have a structure of Formula (VII-B), wherein R^{7a} is

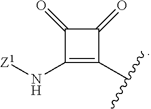

In some embodiments, Lipids of the Disclosure have a structure of Formula (VII-B), wherein R^{7a} is selected from the group consisting of —C(═O)N(R′″)R^{7b}, —C(═S)N(R′″)R^{7b}, and —N═C(R^{7b})(R^{7c}). In some embodiments, Lipids of the Disclosure have a structure of Formula (VII-B), wherein R^{7a} is —C(═O)N(R′″)R^{7b}. In some embodiments, Lipids of the Disclosure have a structure of Formula (VII-B), wherein R^{7a} is —C(═S)N(R′″)R^{7b}. In some embodiments, Lipids of the Disclosure have a structure of Formula (VII-B), wherein R^{7a} is —N═C(R^{7b})(R^{7c}).

In some embodiments, Lipids of the Disclosure have a structure of Formula (VII-B), wherein R^{8a} is selected from the group consisting of —C(═O)N(R″)R^{8b}, —C(═S)N(R′″)R^{8b}, and —N═C(R^{8b})(R^{8c}). In some embodiments, Lipids of the Disclosure have a structure of Formula (VII-B), wherein R^{8a} is —C(═O)N(R″)R^{8b}. In some embodiments, Lipids of the Disclosure have a structure of Formula (VII-B), wherein R^{8a} is —C(═S)N(R′″)R^{8b}. In some embodiments, Lipids of the Disclosure have a structure of Formula (VII-B), wherein R^{8a} is —N═C(R^{8b})(R^{8c}).

In some embodiments, Lipids of the Disclosure have a structure of Formula (VII-B), wherein R^{8a} is

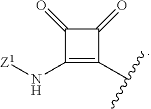

In some embodiments, Lipids of the Disclosure have a structure of Formula (VII-B), wherein R^{9b} is (hydroxy)C₁-C₆ alkyl.

In some embodiments, Lipids of the Disclosure have a structure of Formula (VII-B), wherein R^{10b} is (amino)C₁-C₆ alkyl.

In some embodiments, Lipids of the Disclosure have a structure of Formula (VII-B), wherein R^{11a} is —OR^{11b} or —OC(═O)R^{11b}. In some embodiments, Lipids of the Disclosure have a structure of Formula (VII-B), wherein R^{11a} is —OR^{11b}. In some embodiments, Lipids of the Disclosure have a structure of Formula (VII-B), wherein R^{11a} is —OC(═O)R^{11b}

In some embodiments, Lipids of the Disclosure have a structure of Formula (VII-B), wherein R^{11a} is —N(R″)R^{11b} or —N(R″)C(═O)R^{11b}. In some embodiments, Lipids of the Disclosure have a structure of Formula (VII-B), wherein R^{11a} is —N(R″)R^{11b}. In some embodiments, Lipids of the Disclosure have a structure of Formula (VII-B), wherein R^{11a} is —N(R″)C(═O)R^{11b}.

In some embodiments, Lipids of the Disclosure have a structure of Formula (VII-B), wherein R^{11b} is (amino)C₁-C₆ alkyl.

### Formula (III-C)

In some embodiments, Lipids of the Disclosure have a structure of Formula (III-C):

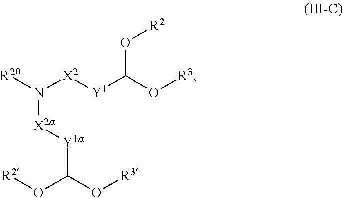

or a pharmaceutically acceptable salt thereof, wherein
R²⁰ is C₁-C₆ alkylenyl-NR²⁰′ C. (O)OR²⁰″;
R²⁰′ is hydrogen or optionally substituted C₁-C₆ alkyl;
R²⁰″ is optionally substituted C₁-C₆ alkyl, phenyl, or benzyl;
Z¹ is optionally substituted C₁-C₆ alkyl;

X² and X^{2a} are independently optionally substituted C₂-C₁₄ alkylenyl;
Y¹ and Y^{1a} are independently

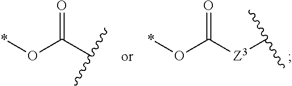

wherein the bond marked with an “*” is attached to X² or X^{2a}.

Z³ is independently optionally substituted C₂-C₆ alkylenyl;
R² and R³ are independently optionally substituted C₄-C₁₄ alkyl; and
R²′ and R³′ are independently optionally substituted C₄-C₁₄ alkyl.

In some embodiments, Lipids of the Disclosure have a structure of Formula (III-C), wherein R²⁰ is —CH₂CH₂CH₂NHC(O)O-t-butyl or —CH₂CH₂CH₂NHC(O)O-benzyl. In some embodiments, Lipids of the Disclosure have a structure of Formula (III-C), wherein R²⁰ is —CH₂CH₂CH₂NHC(O)O-t-butyl. In some embodiments, Lipids of the Disclosure have a structure of Formula (III-C), wherein R²⁰ is —CH₂CH₂CH₂NHC(O)O-benzyl.

In some embodiments, Lipids of the Disclosure have a structure of Formula (III-C), wherein X² and X^{2a} are independently C₄-C₈ alkylenyl (e.g., C₅, C₆, C₇ alkylenyl). In some embodiments, Lipids of the Disclosure have a structure of Formula (III-C), wherein X² is C₆ alkyl. In some embodiments, Lipids of the Disclosure have a structure of Formula (III-C), wherein X^{2a} is C₆ alkyl

In some embodiments, Lipids of the Disclosure have a structure of Formula (III-C), wherein Y¹ and Y^{1a} are

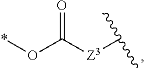

wherein Z³ is C₂-C₄alkylenyl (e.g., C₂ alkylenyl). In some embodiments, Lipids of the Disclosure have a structure of Formula (III-C), wherein Y¹ is

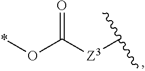

wherein Z³ is C₂-C₄alkylenyl (e.g., C₂ alkylenyl). In some embodiments, Lipids of the Disclosure have a structure of Formula (III-C), wherein Y^{1a} is

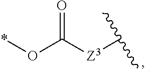

wherein Z³ is C₂-C₄alkylenyl (e.g., C₂ alkylenyl).

In some embodiments, Lipids of the Disclosure have a structure of Formula (III-C), wherein R², R³, R²′ and R³′ are independently optionally substituted C₄-C₁₀ alkyl (e.g., C₆-C₉alkyl, C₆, C₇, C₈, C₉ alkyl). In some embodiments, Lipids of the Disclosure have a structure of Formula (III-C), wherein R² is C₆-C₉alkyl. In some embodiments, Lipids of the Disclosure have a structure of Formula (III-C), wherein R³ is C₆-C₉alkyl. In some embodiments, Lipids of the Disclosure have a structure of Formula (III-C), wherein R²′ is C₆-C₆alkyl. In some embodiments, Lipids of the Disclosure have a structure of Formula (III-C), wherein R³′ is C₆-C₆alkyl.

### Formula (III-D)

In some embodiments, Lipids of the Disclosure have a structure of Formula (III-D):

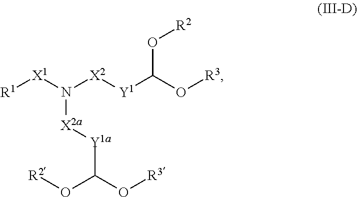

or a pharmaceutically acceptable salt thereof, wherein
R¹ is —OH;
X¹ is optionally substituted C₄ alkylenyl;
X² and X^{2a} are independently optionally substituted C₂-C₁₄ alkylenyl;
Y¹ and Y^{1a} are independently

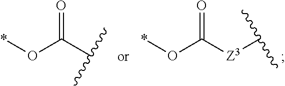

Z³ is independently optionally substituted C₂-C₆ alkylenyl;
R² and R³ are independently optionally substituted C₄-C₁₄ alkyl or C₁-C₂ alkyl substituted with optionally substituted cyclopropyl; or
R²′ and R³′ are independently optionally substituted C₄-C₁₄ alkyl or C₁-C₂ alkyl substituted with optionally substituted cyclopropyl.

In some embodiments, Lipids of the Disclosure have a structure of Formula (III-D), wherein X¹ is C₄ alkylenyl.

In some embodiments, Lipids of the Disclosure have a structure of Formula (III-D), wherein X² and X^{2a} are independently optionally substituted C₄-C₁₀ alkylenyl (e.g., C₅, C₆, C₇, C₈, C₉, or C₁₀ alkylenyl). In some embodiments, Lipids of the Disclosure have a structure of Formula (III-D), wherein X² is C₄-C₁₀ alkylenyl. In some embodiments, Lipids of the Disclosure have a structure of Formula (III-D), wherein X^{2a} is C₄-C₁₀ alkylenyl. In some embodiments, Lipids of the Disclosure have a structure of Formula (III-D), wherein Y¹ and Y^{1a} are independently

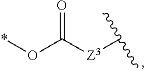

wherein Z³ is independently C₂-C₄ alkylenyl (e.g., C₂, C₄ alkylenyl). In some embodiments, Lipids of the Disclosure have a structure of Formula (III-D), wherein R², R³, R²′ and R³′ are independently C₆-C₁₄ alkyl (e.g., C₆, C₇, C₈, C₉, C₁₀, C₁₁, C₁₂, C₁₃, or C₁₄ alkyl) or C₁-C₂ alkyl substituted with optionally substituted cyclopropyl. In some embodiments, Lipids of the Disclosure have a structure of Formula (III-D), wherein R², R³, R²′ and R³′ are independently C₆-C₁₄ alkyl (e.g., C₆, C₇, C₈, C₉, C₁₀, C₁₁, C₁₂, C₁₃, or C₁₄ alkyl). In some embodiments, Lipids of the Disclosure have a structure of Formula (III-D), wherein R² is C₆-C₁₄ alkyl. In some embodiments, Lipids of the Disclosure have a structure of Formula (III-D), wherein R³ is C₆-C₁₄ alkyl. In some embodiments, Lipids of the Disclosure have a structure of Formula (III-D), wherein R²′ is C₆-C₁₄ alkyl. In some embodiments, Lipids of the Disclosure have a structure of Formula (III-D), wherein R³′ is C₆-C₁₄ alkyl. In some embodiments, Lipids of the Disclosure have a structure of Formula (III-D), wherein R² is C₁-C₂ alkyl substituted with substituted cyclopropyl. In some embodiments, Lipids of the Disclosure have a structure of Formula (III-D), wherein R³ is C₁-C₂ alkyl substituted with substituted cyclopropyl. In some embodiments, Lipids of the Disclosure have a structure of Formula (III-D), wherein R²′ is C₁-C₂ alkyl substituted with substituted cyclopropyl. In some embodiments, Lipids of the Disclosure have a structure of Formula (III-D), wherein R³′ is C₁-C₂ alkyl substituted with substituted cyclopropyl

In some embodiments, Lipids of the Disclosure have a structure of Formula (III-D), wherein R², R³, R²′ and R³′ are independently C₁-C₂ alkyl substituted with cyclopropylene-(C₁-C⁶alkylenyl optionally substituted with cyclopropylene substituted with C₁-C₆alkyl). In some embodiments, Lipids of the Disclosure have a structure of Formula (III-D), wherein R² is C₁-C₂ alkyl substituted with cyclopropylene-(C₁-C₆alkylenyl optionally substituted with cyclopropylene substituted with C₁-C₆alkyl). In some embodiments, Lipids of the Disclosure have a structure of Formula (III-D), wherein R³ is C₁-C₂ alkyl substituted with cyclopropylene-(C₁-C₆alkylenyl optionally substituted with cyclopropylene substituted with C₁-C₆alkyl). In some embodiments, Lipids of the Disclosure have a structure of Formula (III-D), wherein R²′ is C₁-C₂ alkyl substituted with cyclopropylene-(C₁-C₆alkylenyl optionally substituted with cyclopropylene substituted with C₁-C₆alkyl). In some embodiments, Lipids of the Disclosure have a structure of Formula (III-D), wherein R³′ is C₁-C₂ alkyl substituted with cyclopropylene-(C₁-C₆alkylenyl optionally substituted with cyclopropylene substituted with C₁-C₆alkyl).

### Formula (III-E)

In some embodiments, Lipids of the Disclosure have a structure of Formula (III-E):

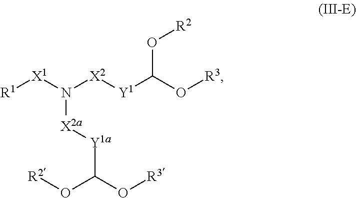

or a pharmaceutically acceptable salt thereof, wherein
R¹ is —OH;
X¹ is branched C₂-C₈ alkylenyl
X² and X^{2a} are independently optionally substituted C₂-C₁₄ alkylenyl;
Y¹ and Y^{1a} are independently

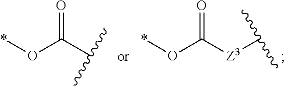

Z³ is independently optionally substituted C₂-C₆ alkylenyl;
R² and R³ are independently optionally substituted C₄-C₁₄ alkyl;
R²′ and R³′ are independently optionally substituted C₄-C₁₄ alkyl.

In some embodiments, Lipids of the Disclosure have a structure of Formula (III-E), wherein X¹ is branched C₆ alkylenyl.

In some embodiments, Lipids of the Disclosure have a structure of Formula (III-E), wherein X² and X^{2a} are independently C₄-C₁₀ alkylenyl (e.g., C₆, C₇, C₈ alkylenyl). In some embodiments, Lipids of the Disclosure have a structure of Formula (III-E), wherein X² is C₄-C₁₀ alkylenyl. In some embodiments, Lipids of the Disclosure have a structure of Formula (III-E), wherein X^{2a} is C₄-C₁₀ alkylenyl

In some embodiments, Lipids of the Disclosure have a structure of Formula (III-E), wherein Y¹ and Y^{1a} are

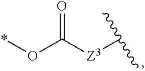

wherein Z³ is independently optionally substituted C₂ alkylenyl. In some embodiments, Lipids of the Disclosure have a structure of Formula (III-E), wherein Y¹ is

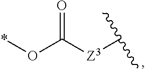

wherein Z³ is independently optionally substituted C₂ alkylenyl. In some embodiments, Lipids of the Disclosure have a structure of Formula (III-E), wherein Y¹ is

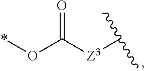

wherein Z³ is independently optionally substituted C₂ wherein Y^{1a} is alkylenyl.

In some embodiments, Lipids of the Disclosure have a structure of Formula (III-E), wherein R², R³, R²′ and R³′ are independently C₆-C₁₂ alkyl (e.g., C₉ alkyl) or C₄-C₁₀ alkyl (e.g., C₄, C₆ alkyl) optionally substituted with C₂-C₈alkenylene (e.g., C₄, C₆ alkenylene). In some embodiments, Lipids of the Disclosure have a structure of Formula (III-E), wherein R² is C₆-C₁₂ alkyl. In some embodiments, Lipids of the Disclosure have a structure of Formula (III-E), wherein R³ is C₆-C₁₂ alkyl. In some embodiments, Lipids of the Disclosure have a structure of Formula (III-E), wherein R²′ is C₆-C₁₂ alkyl. In some embodiments, Lipids of the Disclosure have a structure of Formula (III-E), wherein R³′ is C₆-C₁₂ alkyl. In some embodiments, Lipids of the Disclosure have a structure of Formula (III-E), wherein R² is C₄-C₁₀ alkyl optionally substituted with C₂-C₈alkenylene. In some embodiments, Lipids of the Disclosure have a structure of Formula (III-E), wherein R³ is C₄-C₁₀ alkyl optionally substituted with C₂-C₈alkenylene. In some embodiments, Lipids of the Disclosure have a structure of Formula (III-E), wherein R²′ is C₄-C₁₀ alkyl optionally substituted with C₂-C₈alkenylene. In some embodiments, Lipids of the Disclosure have a structure of Formula (III-E), wherein R³′ is C₄-C₁₀ alkyl optionally substituted with C₂-C₈alkenylene.

### Formula (III-F)

In some embodiments, Lipids of the Disclosure have a structure of Formula (III-F):

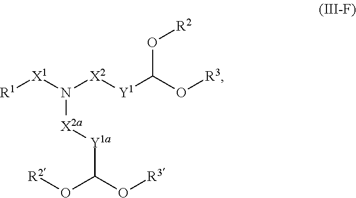

or a pharmaceutically acceptable salt thereof, wherein
R¹ is —OH;
X¹ is optionally substituted C₂-C₆ alkylenyl;
X² and X^{2a} are independently optionally substituted C₂-C₁₄ alkylenyl;
each of Y¹ and Y^{1a} is a bond;
R² and R³ are independently optionally substituted C₄-C₁₄ alkyl; and
R²′ and R³′ are independently optionally substituted C₄-C₁₄ alkyl.

In some embodiments, Lipids of the Disclosure have a structure of Formula (III-E), wherein X¹ is C₄ alkylenyl.

In some embodiments, Lipids of the Disclosure have a structure of Formula (III-E), wherein X² and X^{2a} are independently C₄-C₁₀ alkylenyl (e.g., C₆-C₈ alkylenyl, C₆, C₇, C₈ alkylenyl). In some embodiments, Lipids of the Disclosure have a structure of Formula (III-E), wherein X² is C₄-C₁₀ alkylenyl. In some embodiments, Lipids of the Disclosure have a structure of Formula (III-E), wherein X^{2a} is C₄-C₁₀ alkylenyl.

In some embodiments, Lipids of the Disclosure have a structure of Formula (III-E), wherein R², R³, R²′ and R³′ are independently C₆-C₁₀ alkyl (e.g., C₇. C₈ alkyl). In some embodiments, Lipids of the Disclosure have a structure of Formula (III-E), wherein R² is C₆-C₁₀ alkyl. In some embodiments, Lipids of the Disclosure have a structure of Formula (III-E), wherein R³ is C₆-C₁₀ alkyl. In some embodiments, Lipids of the Disclosure have a structure of Formula (III-E), wherein R²′ is C₆-C₁₀ alkyl. In some embodiments, Lipids of the Disclosure have a structure of Formula (III-E), wherein R³′ is C₆-C₁₀ alkyl.

### Formula (VIII-B)

In some embodiments, Lipids of the Disclosure have a structure of Formula (VIII-B):

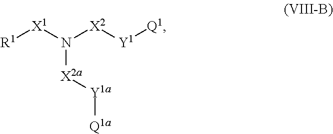

or a pharmaceutically acceptable salt thereof, wherein:
X¹ is a bond,
R¹ is C₁-C₆ alkyl,
X² is is C₂-C₆ alkylenyl,
X^{2a} is C₂-C₁₄ alkylenyl,
wherein X² or X^{2a} is substituted with OH or C₁₋₄alkylenyl-OH,
Y¹ is

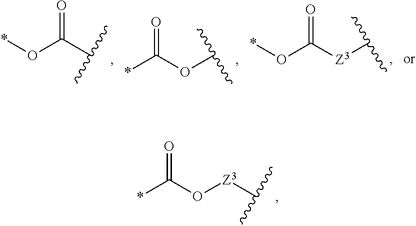

wherein the bond marked with an “*” is attached to X²;
Y^{1a} is

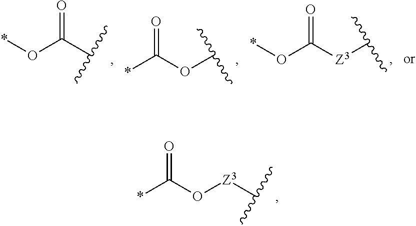

wherein the bond marked with an “*” is attached to X²a,
each Z³ is independently optionally substituted C₁-C₆ alkylenyl or optionally substituted C₂-C₁₄ alkenylenyl;
Q¹ is —C(R²)(R³)(R¹²);
Q^{1a} is —C(R²′)(R³′)(R¹²′);
R², R³, and R¹² are independently hydrogen, optionally substituted C₁-C₁₄ alkyl, or optionally substituted C₂-C₁₄ alkenylenyl, and
R²′, R³′, and R¹²′ are independently hydrogen, optionally substituted C₁-C₁₄ alkyl, or optionally substituted C₂-C₁₄ alkenylenyl.

In some embodiments, Lipids of the Disclosure have a structure of Formula (VIII-B), wherein R¹ is methyl.

In some embodiments, Lipids of the Disclosure have a structure of Formula (VIII-B), wherein X² is C₄, C₅, or C₆ alkylenyl.

In some embodiments, Lipids of the Disclosure have a structure of Formula (VIII-B), wherein X^{2a} is C₄-C₈ alkylenyl (e.g., C₅, C₆, or C₇ alkylenyl).

In some embodiments, Lipids of the Disclosure have a structure of Formula (VIII-B), wherein Y¹ is

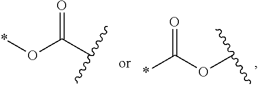 [SMILES: *OC(*)=O ; *OC(*)=O]

### and Y^{1a} is

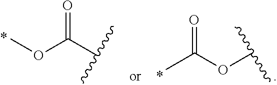

In some embodiments, Lipids of the Disclosure have a structure of Formula (VIII-B), wherein Y¹ is

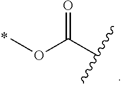 [SMILES: *C(=O)OC]

In some embodiments, Lipids of the Disclosure have a structure of Formula (VIII-B), wherein Y¹ is

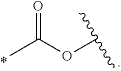 [SMILES: *OC(C)=O]

In some embodiments, Lipids of the Disclosure have a structure of Formula (VIII-B), wherein Y^{1a} is

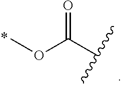 [SMILES: *C(=O)OC]

In some embodiments, Lipids of the Disclosure have a structure of Formula (VIII-B), wherein Y^{1a} is

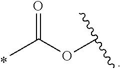 [SMILES: *OC(C)=O]

In some embodiments, Lipids of the Disclosure have a structure of Formula (VIII-B), wherein R², R³, R¹², R²′, R³′, and R¹²′ are independently hydrogen or C₅-C₁₂ alkyl (e.g., C₆, C₇, C₈, C₉, C₁₀, C₁₁ alkyl). In some embodiments, Lipids of the Disclosure have a structure of Formula (VIII-B), wherein R² is hydrogen. In some embodiments, Lipids of the Disclosure have a structure of Formula (VIII-B), wherein R³ is hydrogen. In some embodiments, Lipids of the Disclosure have a structure of Formula (VIII-B), wherein R²′ is hydrogen. In some embodiments, Lipids of the Disclosure have a structure of Formula (VIII-B), wherein R³′ is hydrogen. In some embodiments, Lipids of the Disclosure have a structure of Formula (VIII-B), wherein R² is C₅-C₁₂ alkyl. In some embodiments, Lipids of the Disclosure have a structure of Formula (VIII-B), wherein R³ is C₅-C₁₂ alkyl. In some embodiments, Lipids of the Disclosure have a structure of Formula (VIII-B), wherein R²′ is C₅-C₁₂ alkyl. In some embodiments, Lipids of the Disclosure have a structure of Formula (VIII-B), wherein R³′ is C₅-C₁₂ alkyl.

### Formula (X)

In some embodiments, Lipids of the Disclosure have a structure of Formula (X):

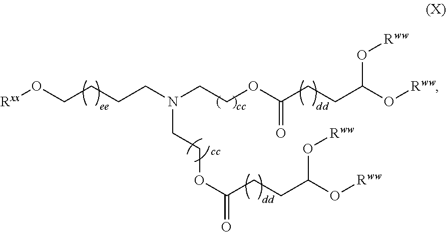

or a pharmaceutically acceptable salt thereof, wherein
each cc is independently selected from 3 to 9;
R^{XX} is selected from hydrogen and optionally substituted C₁-C₆ alkyl; and
(i) ee is 1,
   each dd is independently selected from 1 to 4; and
each R^{WW} is independently selected from the group consisting of C₄-C₁₄ alkyl, branched C₄-C₁₂ alkenyl, C₄-C₁₂ alkenyl comprising at least two double bonds, and C₉-C₁₂ alkenyl, wherein any —(CH₂)₂— of the C₄-C₁₄ alkyl can be optionally replaced with C₂-C₆ cycloalkylenyl;
(ii) ee is 0,
   each dd is 1; and
   each R^{WW} is linear C₄-C₁₂ alkyl.

In some embodiments, Lipids of the Disclosure have a structure of Formula (X), wherein R^{XX} is H. In some embodiments, Lipids of the Disclosure have a structure of Formula (X), wherein R^{XX} is optionally substituted C₁-C₆ alkyl. In some embodiments, Lipids of the Disclosure have a structure of Formula (X), wherein R^{XX} is C₁ alkyl. In some embodiments, Lipids of the Disclosure have a structure of Formula (X), wherein R^{XX} is C₂ alkyl. In some embodiments, Lipids of the Disclosure have a structure of Formula (X), wherein R^{XX} is C₃ alkyl. In some embodiments, Lipids of the Disclosure have a structure of Formula (X), wherein R^{XX} is C₄ alkyl. In some embodiments, Lipids of the Disclosure have a structure of Formula (X), wherein R^{XX} is C₅ alkyl. In some embodiments, Lipids of the Disclosure have a structure of Formula (X), wherein R^{XX} is C₆ alkyl.

In some embodiments, Lipids of the Disclosure have a structure of Formula (X), wherein each R^{WW} is independently selected from the group consisting of C₄-C₁₄ alkyl, branched C₄-C₁₂ alkenyl, C₄-C₁₂ alkenyl comprising at least two double bonds, and C₉-C₁₂ alkenyl, wherein any —(CH₂)₂— of the C₄-C₁₄ alkyl can be optionally replaced with C₂-C₆ cycloalkylenyl. In some embodiments, Lipids of the Disclosure have a structure of Formula (X), wherein each R^{WW} is C₄-C₁₄ alkyl, wherein any —(CH₂)₂— of the C₄-C₁₄ alkyl can be optionally replaced with C₂-C₆ cycloalkylenyl. In some embodiments, Lipids of the Disclosure have a structure of Formula (X), wherein each R^{WW} is C₄-C₁₄ alkyl, wherein any —(CH₂)₂— of the C₄-C₁₄ alkyl can be optionally replaced with cyclopropylene. In some embodiments, Lipids of the Disclosure have a structure of Formula (X), wherein each R^{WW} is branched C₄-C₁₂ alkenyl. In some embodiments, Lipids of the Disclosure have a structure of Formula (X), wherein each R^{WW} is C₄-C₁₂ alkenyl comprising at least two double bonds. In some embodiments, Lipids of the Disclosure have a structure of Formula (X), wherein each R^{WW} is C₉-C₁₂ alkenyl. In some embodiments, Lipids of the Disclosure have a structure of Formula (X), wherein each R^{WW} is linear C₄-C₁₂ alkyl. In some embodiments, Lipids of the Disclosure have a structure of Formula (X), wherein each R^{WW} is independently selected from the group consisting of C₆-C₁₄ alkyl, branched C₈-C₁₂ alkenyl, C₈-C₁₂ alkenyl comprising at least two double bonds, and C₉-C₁₂ alkenyl, wherein any —(CH₂)₂— of the C₆-C₁₄ alkyl can be optionally replaced with cyclopropylene. In some embodiments, Lipids of the Disclosure have a structure of Formula (X), wherein each R^{WW} is C₆-C₁₄ alkyl, wherein any —(CH₂)₂— of the C₆-C₁₄ alkyl can be optionally replaced with cyclopropylene. In some embodiments, Lipids of the Disclosure have a structure of Formula (X), wherein each R^{WW} is branched C₈-C₁₂ alkenyl, e.g., (linear or branched C₃-C₅ alkylenyl)-(branched C₅-C₇alkenyl), e.g., (branched C₅ alkylenyl)-(branched C₅alkenyl), e.g.,

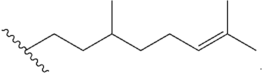 [SMILES: *CCC(C)CCC=C(C)C]

In some embodiments, Lipids of the Disclosure have a structure of Formula (X), wherein each R^{WW} is C₈-C₁₂ alkenyl comprising at least two double bonds. In some embodiments, Lipids of the Disclosure have a structure of Formula (X), wherein each R^{WW} is C₉-C₁₂ alkenyl.

In some embodiments, Lipids of the Disclosure have a structure of Formula (X), wherein each R^{WW} is independently selected from the group consisting of C₆-C₁₄ alkyl (e.g., C₆, C₈, C₉, C₁₀, C₁₁, C₁₃ alkyl), wherein any —(CH₂)₂— of the C₆-C₁₄ alkyl can be optionally replaced with cyclopropylene.

In some embodiments, Lipids of the Disclosure have a structure of Formula (X), wherein each R^{WW} is independently branched C₈-C₁₂ alkenyl (e.g., branched C₁₀ alkenyl).

In some embodiments, Lipids of the Disclosure have a structure of Formula (X), wherein each R^{WW} is independently C₈-C₁₂ alkenyl comprising at least two double bonds (e.g., C₉ or C₁₀ alkenyl comprising two double bonds).

In some embodiments, Lipids of the Disclosure have a structure of Formula (X), wherein each R^{WW} is independently (C₁ alkylenyl)-(cyclopropylene-C₆ alkyl) or (C₂ alkylenyl)-(cyclopropylene-C₂ alkyl). In some embodiments, Lipids of the Disclosure have a structure of Formula (X), wherein each R^{WW} is independently (C₁ alkylenyl)-(cyclopropylene-C₆ alkyl). In some embodiments, Lipids of the Disclosure have a structure of Formula (X), wherein each R^{WW} is independently (C₂ alkylenyl)-(cyclopropylene-C₂ alkyl).

In some embodiments, Lipids of the Disclosure have a structure of Formula (X), wherein each R^{WW} is C₄ alkyl. In some embodiments, Lipids of the Disclosure have a structure of Formula (X), wherein each R^{WW} is C₅ alkyl. In some embodiments, Lipids of the Disclosure have a structure of Formula (X), wherein each R^{WW} is C₆ alkyl. In some embodiments, Lipids of the Disclosure have a structure of Formula (X), wherein each R^{WW} is C₇ alkyl. In some embodiments, Lipids of the Disclosure have a structure of Formula (X), wherein each R^{WW} is C₅ alkyl. In some embodiments, Lipids of the Disclosure have a structure of Formula (X), wherein each R^{WW} is C₉ alkyl. In some embodiments, Lipids of the Disclosure have a structure of Formula (X), wherein each R^{WW} is C₁₀ alkyl. In some embodiments, Lipids of the Disclosure have a structure of Formula (X), wherein each R^{WW} is C₁₁ alkyl. In some embodiments, Lipids of the Disclosure have a structure of Formula (X), wherein each R^{WW} is C₁₂ alkyl. In some embodiments, Lipids of the Disclosure have a structure of Formula (X), wherein each R^{WW} is C₁₃ alkyl. In some embodiments, Lipids of the Disclosure have a structure of Formula (X), wherein each R^{WW} is C₁₄ alkyl.

In some embodiments, Lipids of the Disclosure have a structure of Formula (X), wherein each R^{WW} is C₉ alkenyl. In some embodiments, Lipids of the Disclosure have a structure of Formula (X), wherein each R^{WW} is C₁₀ alkenyl. In some embodiments, Lipids of the Disclosure have a structure of Formula (X), wherein each R^{WW} is C₁₁ alkenyl. In some embodiments, Lipids of the Disclosure have a structure of Formula (X), wherein each R^{WW} is C₁₂ alkenyl.

In some embodiments, Lipids of the Disclosure have a structure of Formula (X), wherein each R^{WW} is C₈ alkenyl comprising at least two double bonds. In some embodiments, Lipids of the Disclosure have a structure of Formula (X), wherein each R^{WW} is C₉ alkenyl comprising at least two double bonds. In some embodiments, Lipids of the Disclosure have a structure of Formula (X), wherein each R^{WW} is C₁₀ alkenyl comprising at least two double bonds. In some embodiments, Lipids of the Disclosure have a structure of Formula (X), wherein each R^{WW} is C₁₁ alkenyl comprising at least two double bonds. In some embodiments, Lipids of the Disclosure have a structure of Formula (X), wherein each R^{WW} is C₁₂ alkenyl comprising at least two double bonds. In some embodiments, Lipids of the Disclosure have a structure of Formula (X), wherein each R^{WW} is C₁₃ alkenyl comprising at least two double bonds. In some embodiments, Lipids of the Disclosure have a structure of Formula (X), wherein each R^{WW} is C₁₄ alkenyl comprising at least two double bonds.

In some embodiments, Lipids of the Disclosure have a structure of Formula (X), wherein each R^{WW} is C₉ alkyl, wherein one —(CH₂)₂— of the C₉ alkyl is replaced with C₂-C₆ cycloalkylenyl. In some embodiments, Lipids of the Disclosure have a structure of Formula (X), wherein each R^{WW} is C₉ alkyl, wherein one —(CH₂)₂— of the C₉ alkyl is replaced with cyclopropylene. In some embodiments, Lipids of the Disclosure have a structure of Formula (X), wherein each R^{WW} is C₉ alkyl, wherein two —(CH₂)₂— of the C₉ alkyl are replaced with C₂-C₆ cycloalkylenyl. In some embodiments, Lipids of the Disclosure have a structure of Formula (X), wherein each R^{WW} is C₉ alkyl, wherein two —(CH₂)₂— of the C₉ alkyl are replaced with cyclopropylene.

In some embodiments, Lipids of the Disclosure have a structure of Formula (X), wherein each R^{WW} is linear C₄ alkyl. In some embodiments, Lipids of the Disclosure have a structure of Formula (X), wherein each R^{WW} is linear C₅ alkyl. In some embodiments, Lipids of the Disclosure have a structure of Formula (X), wherein each R^{WW} is linear C₆ alkyl. In some embodiments, Lipids of the Disclosure have a structure of Formula (X), wherein each R^{WW} is linear C₇ alkyl. In some embodiments, Lipids of the Disclosure have a structure of Formula (X), wherein each R^{WW} is linear C₈ alkyl. In some embodiments, Lipids of the Disclosure have a structure of Formula (X), wherein each R^{WW} is linear C₉ alkyl. In some embodiments, Lipids of the Disclosure have a structure of Formula (X), wherein each R^{WW} is linear C₁₀ alkyl. In some embodiments, Lipids of the Disclosure have a structure of Formula (X), wherein each R^{WW} is linear C₁₁ alkyl. In some embodiments, Lipids of the Disclosure have a structure of Formula (X), wherein each R^{WW} is linear C₁₂ alkyl. In some embodiments, Lipids of the Disclosure have a structure of Formula (X), wherein each R^{WW} is linear C₁₃ alkyl. In some embodiments, Lipids of the Disclosure have a structure of Formula (X), wherein each R^{WW} is linear C₁₄ alkyl.

In some embodiments, Lipids of the Disclosure have a structure of Formula (X), wherein each R^{WW} is branched C₈ alkenyl. In some embodiments, Lipids of the Disclosure have a structure of Formula (X), wherein each R^{WW} is branched C₉ alkenyl. In some embodiments, Lipids of the Disclosure have a structure of Formula (X), wherein each R^{WW} is branched C₁₀ alkenyl. In some embodiments, Lipids of the Disclosure have a structure of Formula (X), wherein each R^{WW} is branched C₁₁ alkenyl. In some embodiments, Lipids of the Disclosure have a structure of Formula (X), wherein each R^{WW} is branched C₁₂ alkenyl.

In some embodiments, Lipids of the Disclosure have a structure of Formula (X), wherein each cc is independently selected from 3 to 7. In some embodiments, Lipids of the Disclosure have a structure of Formula (X), wherein each cc is 3. In some embodiments, Lipids of the Disclosure have a structure of Formula (X), wherein each cc is 4. In some embodiments, Lipids of the Disclosure have a structure of Formula (X), wherein each cc is 5. In some embodiments, Lipids of the Disclosure have a structure of Formula (X), wherein each cc is 6. In some embodiments, Lipids of the Disclosure have a structure of Formula (X), wherein each cc is 7. In some embodiments, Lipids of the Disclosure have a structure of Formula (X), wherein each cc is 8. In some embodiments, Lipids of the Disclosure have a structure of Formula (X), wherein each cc is 9.

In some embodiments, Lipids of the Disclosure have a structure of Formula (X), wherein each dd is independently selected from 1 to 4. In some embodiments, Lipids of the Disclosure have a structure of Formula (X), wherein each dd is 1. In some embodiments, Lipids of the Disclosure have a structure of Formula (X), wherein each dd is 2. In some embodiments, Lipids of the Disclosure have a structure of Formula (X), wherein each dd is 3. In some embodiments, Lipids of the Disclosure have a structure of Formula (X), wherein each dd is 4.

In some embodiments, Lipids of the Disclosure have a structure of Formula (X), wherein ee is 1.

In some embodiments, Lipids of the Disclosure have a structure of Formula (X), wherein ee is 0.

### Formula (X-A)

In some embodiments, Lipids of the Disclosure have a structure of Formula (X), wherein the Lipids of the Disclosure have a structure of Formula (X-A):

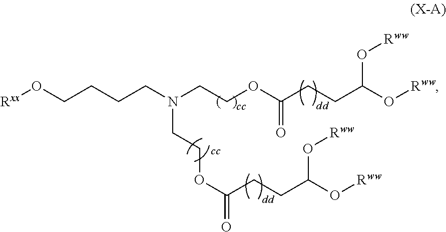

or a pharmaceutically acceptable salt thereof, wherein
each cc is independently selected from 3 to 7;
each dd is independently selected from 1 to 4;
R^{XX} is selected from hydrogen and optionally substituted C₁-C₆ alkyl; and
each R^{WW} is independently selected from the group consisting of C₄-C₁₄ alkyl or (linear or branched C₃-C₅ alkylenyl)-(branched C₅-C₇alkenyl).

In some embodiments, Lipids of the Disclosure have a structure of Formula (X-A), wherein R^{XX} is hydrogen. In some embodiments, Lipids of the Disclosure have a structure of Formula (X-A), wherein RX is C₁ alkyl. In some embodiments, Lipids of the Disclosure have a structure of Formula (X-A), wherein R^{xX} is C₂ alkyl. In some embodiments, Lipids of the Disclosure have a structure of Formula (X-A), wherein RX is C₃ alkyl. In some embodiments, Lipids of the Disclosure have a structure of Formula (X-A), wherein R^{XX} is C₄ alkyl. In some embodiments, Lipids of the Disclosure have a structure of Formula (X-A), wherein R^{XX} is C₅ alkyl. In some embodiments, Lipids of the Disclosure have a structure of Formula (X-A), wherein R^{XX} is C₆ alkyl.

In some embodiments, Lipids of the Disclosure have a structure of Formula (X-A), wherein each cc is 4, 5, 6, or 7. In some embodiments, Lipids of the Disclosure have a structure of Formula (X-A), wherein each cc is 3. In some embodiments, Lipids of the Disclosure have a structure of Formula (X-A), wherein each cc is 4. In some embodiments, Lipids of the Disclosure have a structure of Formula (X-A), wherein each cc is 5. In some embodiments, Lipids of the Disclosure have a structure of Formula (X-A), wherein each cc is 6. In some embodiments, Lipids of the Disclosure have a structure of Formula (X-A), wherein each cc is 7.

In some embodiments, Lipids of the Disclosure have a structure of Formula (X-A), wherein each dd is 1 or 3. In some embodiments, Lipids of the Disclosure have a structure of Formula (X-A), wherein each dd is 1. In some embodiments, Lipids of the Disclosure have a structure of Formula (X-A), wherein each dd is 2. In some embodiments, Lipids of the Disclosure have a structure of Formula (X-A), wherein each dd is 3. In some embodiments, Lipids of the Disclosure have a structure of Formula (X-A), wherein each dd is 4.

In some embodiments, Lipids of the Disclosure have a structure of Formula (X-A), wherein each R^{WW} is C₄-C₁₄ alkyl. In some embodiments, Lipids of the Disclosure have a structure of Formula (X-A), wherein each R^{WW} is C₄ alkyl. In some embodiments, Lipids of the Disclosure have a structure of Formula (X-A), wherein each R^{WW} is C₅ alkyl. In some embodiments, Lipids of the Disclosure have a structure of Formula (X-A), wherein each R^{WW} is C₆ alkyl. In some embodiments, Lipids of the Disclosure have a structure of Formula (X-A), wherein each R^{WW} is C₇ alkyl. In some embodiments, Lipids of the Disclosure have a structure of Formula (X-A), wherein each R^{WW} is C₈ alkyl. In some embodiments, Lipids of the Disclosure have a structure of Formula (X-A), wherein each R^{WW} is C₉ alkyl. In some embodiments, Lipids of the Disclosure have a structure of Formula (X-A), wherein each R^{WW} is C₁₀ alkyl. In some embodiments, Lipids of the Disclosure have a structure of Formula (X-A), wherein each R^{WW} is C₁₁ alkyl. In some embodiments, Lipids of the Disclosure have a structure of Formula (X-A), wherein each R^{WW} is C₁₂ alkyl. In some embodiments, Lipids of the Disclosure have a structure of Formula (X-A), wherein each R^{WW} is C₁₃ alkyl. In some embodiments, Lipids of the Disclosure have a structure of Formula (X-A), wherein each R^{WW} is C₁₄ alkyl.

In some embodiments, Lipids of the Disclosure have a structure of Formula (X-A), wherein each R^{WW} is (linear or branched C₃-C₅ alkylenyl)-(branched C₅-C₇alkenyl), e.g., (branched C₅ alkylenyl)-(branched C₅alkenyl), e.g.,

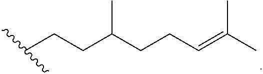 [SMILES: *CCC(C)CCC=C(C)C]

In some embodiments, Lipids of the Disclosure comprise an acyclic core. In some embodiments, Lipids of the Disclosure are selected from any lipid in Table (I) below or a pharmaceutically acceptable salt thereof:

Table (I)
Non-Limiting Examples of Ionizable Lipids with an Acyclic Core

| | Compound |
|---|---|
| Structure | No. |
| 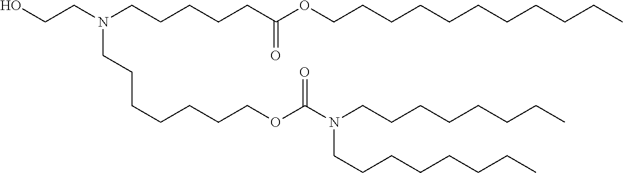 [SMILES: CCCCCCCCCCCOC(=O)CCCCCN(CCO)CCCCCCCOC(=O)N(CCCCCCCC)CCCCCCCC] | 1 |
| 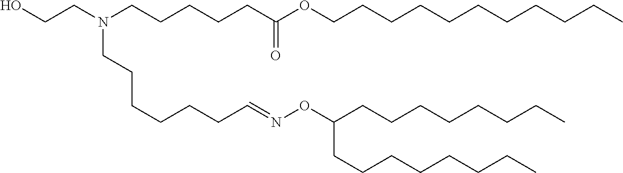 [SMILES: CCCCCCCCCCCOC(=O)CCCCCN(CCO)CCCCCC/C=N/OC(CCCCCCCC)CCCCCCCC] | 2 |
| 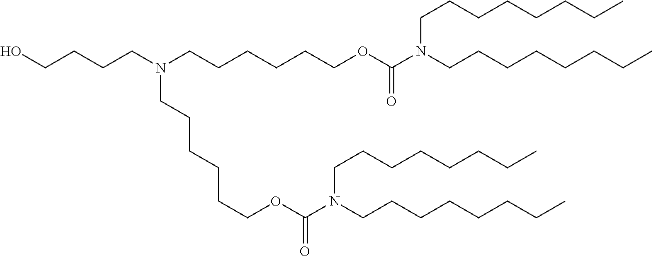 [SMILES: CCCCCCCCN(CCCCCCCC)C(=O)OCCCCCCN(CCCCO)CCCCCCOC(=O)N(CCCCCCCC)CCCCCCCC] | 3 |
| 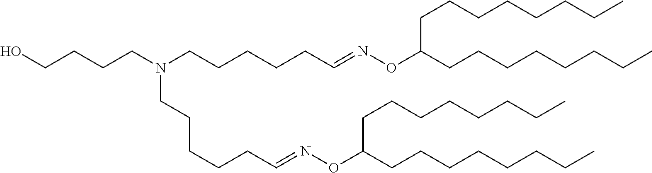 [SMILES: CCCCCCCCC(CCCCCCCC)O/N=C/CCCCCN(CCCCO)CCCCC/C=N/OC(CCCCCCCC)CCCCCCCC] | 4 |
| 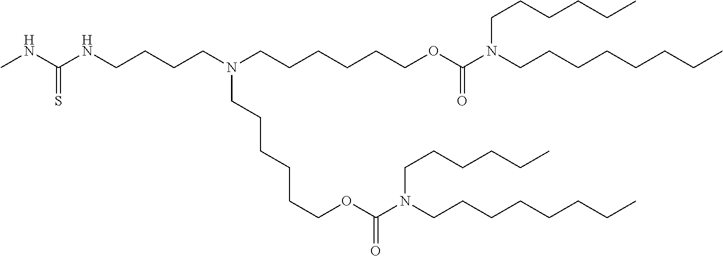 [SMILES: CCCCCCCCN(CCCCCC)C(=O)OCCCCCCN(CCCCCCOC(=O)N(CCCCCC)CCCCCCCC)CCCCNC(=S)NC] | 5 |
| 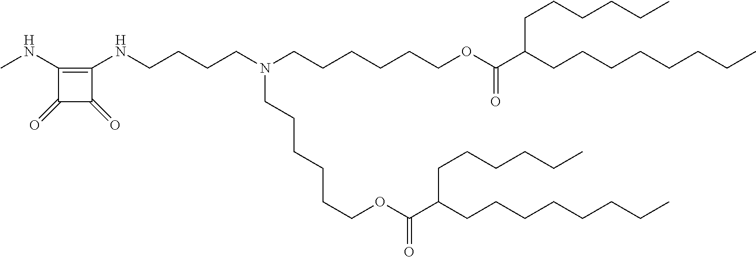 [SMILES: CCCCCCCCC(CCCCCC)C(=O)OCCCCCCN(CCCCCCOC(=O)C(CCCCCC)CCCCCCCC)CCCCNc1c(NC)c(=O)c1=O] | 6 |
| 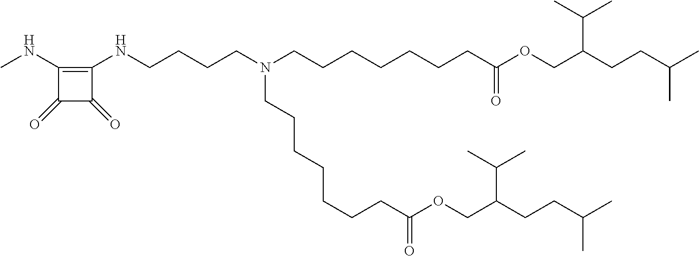 [SMILES: CNc1c(NCCCCN(CCCCCCCC(=O)OCC(CCC(C)C)C(C)C)CCCCCCCC(=O)OCC(CCC(C)C)C(C)C)c(=O)c1=O] | 7 |
| 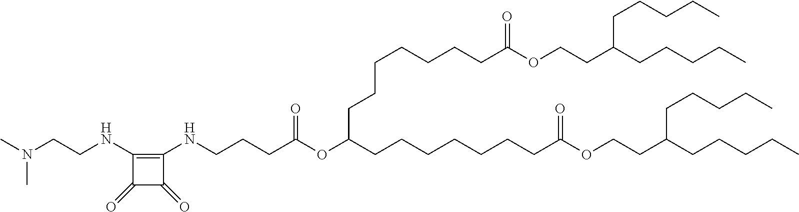 [SMILES: CCCCCC(CCCCC)CCOC(=O)CCCCCCCC(CCCCCCCC(=O)OCCC(CCCCC)CCCCC)OC(=O)CCCNc1c(NCCN(C)C)c(=O)c1=O] | 8 |
| 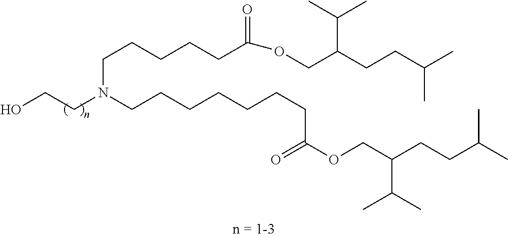 | 9 |
| 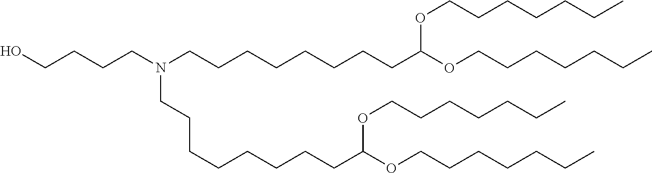 [SMILES: CCCCCCCOC(CCCCCCCCN(CCCCO)CCCCCCCCC(OCCCCCCC)OCCCCCCC)OCCCCCCC] | 10 |
| 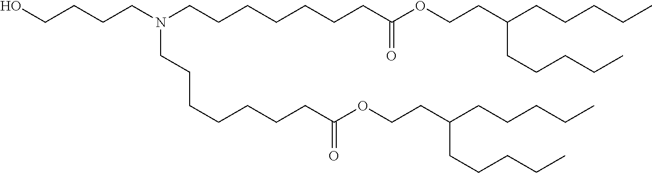 [SMILES: CCCCCC(CCCCC)CCOC(=O)CCCCCCCN(CCCCO)CCCCCCCC(=O)OCCC(CCCCC)CCCCC] | 11 |
| 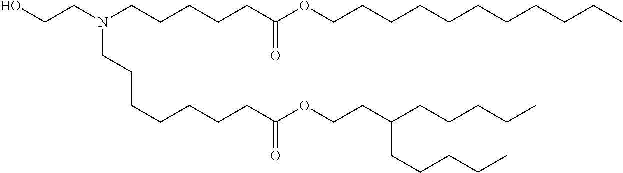 [SMILES: CCCCCCCCCCCOC(=O)CCCCCN(CCO)CCCCCCCC(=O)OCCC(CCCCC)CCCCC] | 13 |
| 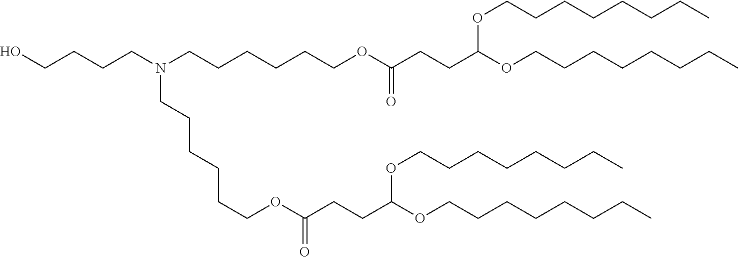 [SMILES: CCCCCCCCOC(CCC(=O)OCCCCCCN(CCCCO)CCCCCCOC(=O)CCC(OCCCCCCCC)OCCCCCCCC)OCCCCCCCC] | 14 |
| 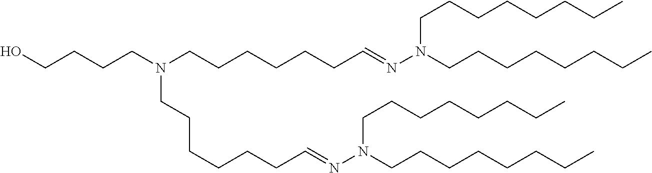 [SMILES: CCCCCCCCN(CCCCCCCC)/N=C/CCCCCCN(CCCCO)CCCCCC/C=N/N(CCCCCCCC)CCCCCCCC] | 15 |
| 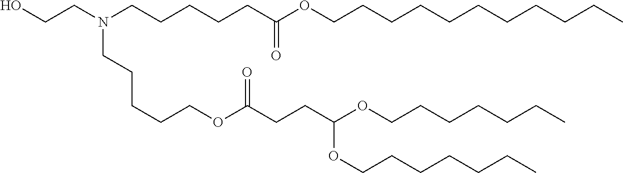 [SMILES: CCCCCCCCCCCOC(=O)CCCCCN(CCO)CCCCCOC(=O)CCC(OCCCCCCC)OCCCCCCC] | 16 |
| 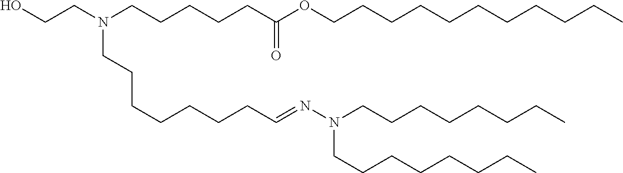 [SMILES: CCCCCCCCCCCOC(=O)CCCCCN(CCO)CCCCCCC/C=N/N(CCCCCCCC)CCCCCCCC] | 17 |
| 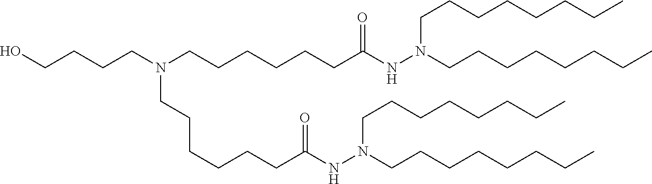 [SMILES: CCCCCCCCN(CCCCCCCC)NC(=O)CCCCCCN(CCCCO)CCCCCCC(=O)NN(CCCCCCCC)CCCCCCCC] | 18 |
| 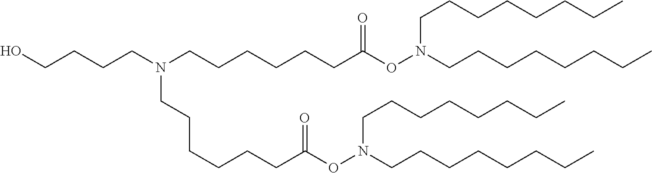 [SMILES: CCCCCCCCN(CCCCCCCC)OC(=O)CCCCCCN(CCCCO)CCCCCCC(=O)ON(CCCCCCCC)CCCCCCCC] | 19 |
| 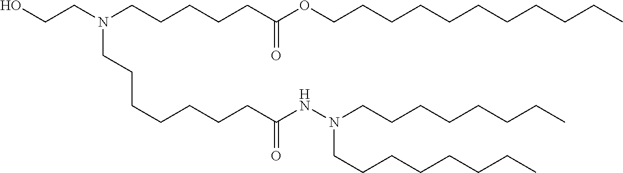 [SMILES: CCCCCCCCCCCOC(=O)CCCCCN(CCO)CCCCCCCC(=O)NN(CCCCCCCC)CCCCCCCC] | 20 |
| 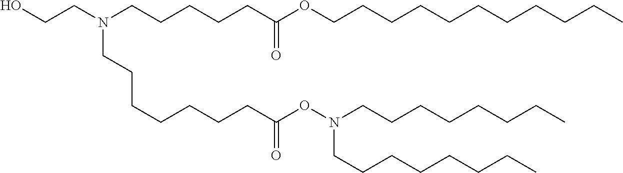 [SMILES: CCCCCCCCCCCOC(=O)CCCCCN(CCO)CCCCCCCC(=O)ON(CCCCCCCC)CCCCCCCC] | 21 |
| 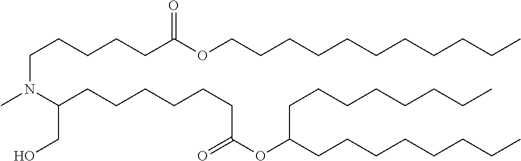 [SMILES: CCCCCCCCCCCOC(=O)CCCCCN(C)C(CO)CCCCCCC(=O)OC(CCCCCCCC)CCCCCCCC] | 22 |
| 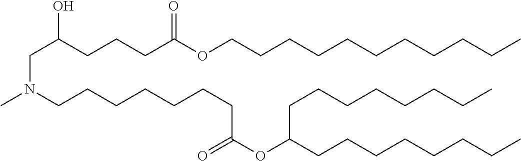 [SMILES: CCCCCCCCCCCOC(=O)CCCC(O)CN(C)CCCCCCCC(=O)OC(CCCCCCCC)CCCCCCCC] | 23 |
| 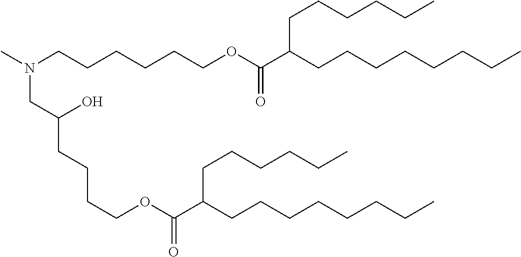 [SMILES: CCCCCCCCC(CCCCCC)C(=O)OCCCCCCN(C)CC(O)CCCCOC(=O)C(CCCCCC)CCCCCCCC] | 24 |
| 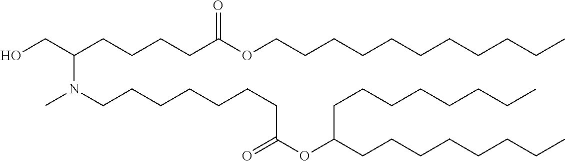 [SMILES: CCCCCCCCCCCOC(=O)CCCCC(CO)N(C)CCCCCCCC(=O)OC(CCCCCCCC)CCCCCCCC] | 25 |
| 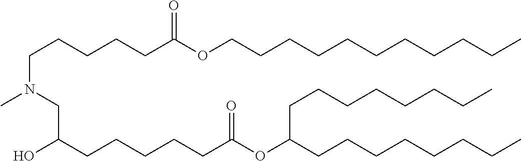 [SMILES: CCCCCCCCCCCOC(=O)CCCCCN(C)CC(O)CCCCCC(=O)OC(CCCCCCCC)CCCCCCCC] | 26 |
| 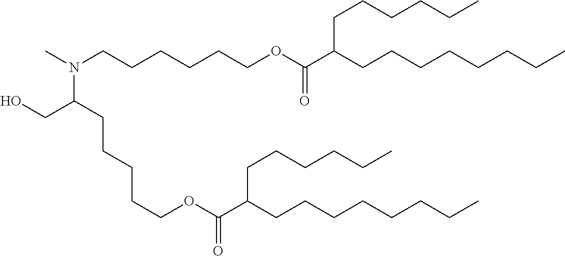 [SMILES: CCCCCCCCC(CCCCCC)C(=O)OCCCCCCN(C)C(CO)CCCCCOC(=O)C(CCCCCC)CCCCCCCC] | 27 |
| 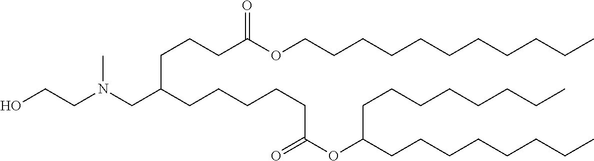 [SMILES: CCCCCCCCCCCOC(=O)CCCC(CCCCCC(=O)OC(CCCCCCCC)CCCCCCCC)CN(C)CCO] | 28 |
| 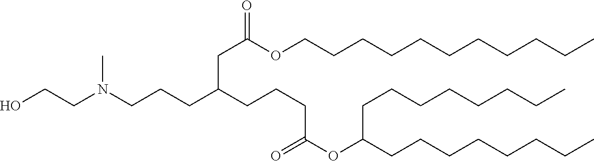 [SMILES: CCCCCCCCCCCOC(=O)CC(CCCC(=O)OC(CCCCCCCC)CCCCCCCC)CCCN(C)CCO] | 29 |
| 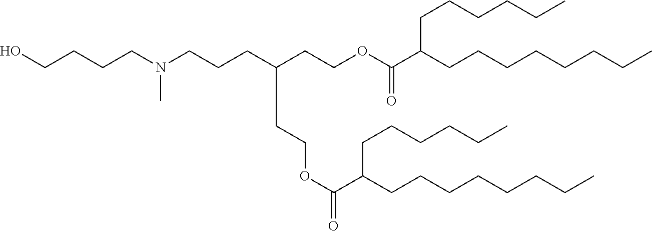 [SMILES: CCCCCCCCC(CCCCCC)C(=O)OCCC(CCCN(C)CCCCO)CCOC(=O)C(CCCCCC)CCCCCCCC] | 30 |
| 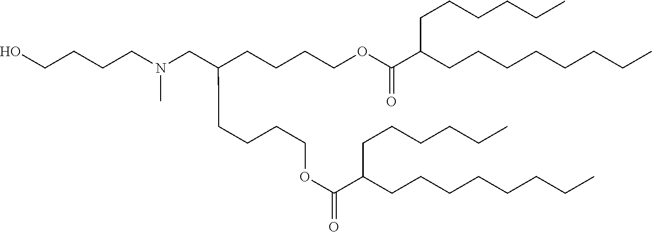 [SMILES: CCCCCCCCC(CCCCCC)C(=O)OCCCCC(CCCCOC(=O)C(CCCCCC)CCCCCCCC)CN(C)CCCCO] | 31 |
| 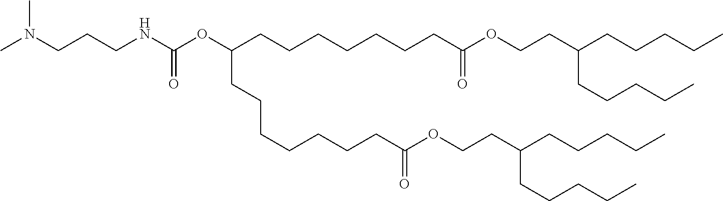 [SMILES: CCCCCC(CCCCC)CCOC(=O)CCCCCCCC(CCCCCCCC(=O)OCCC(CCCCC)CCCCC)OC(=O)NCCCN(C)C] | 32 |
| 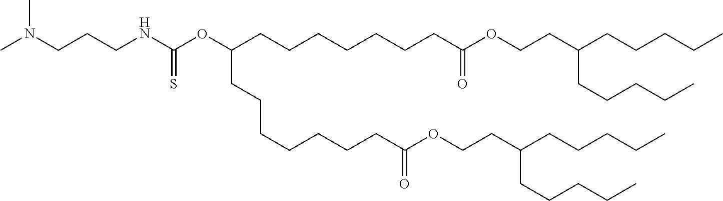 [SMILES: CCCCCC(CCCCC)CCOC(=O)CCCCCCCC(CCCCCCCC(=O)OCCC(CCCCC)CCCCC)OC(=S)NCCCN(C)C] | 33 |
| 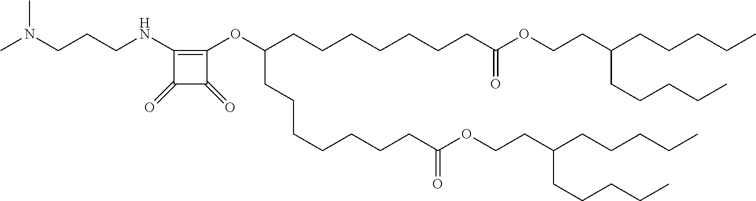 [SMILES: CCCCCC(CCCCC)CCOC(=O)CCCCCCCC(CCCCCCCC(=O)OCCC(CCCCC)CCCCC)Oc1c(NCCCN(C)C)c(=O)c1=O] | 34 |
| 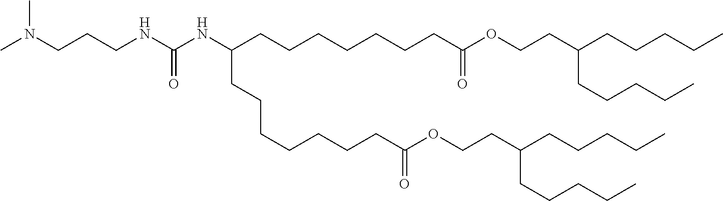 [SMILES: CCCCCC(CCCCC)CCOC(=O)CCCCCCCC(CCCCCCCC(=O)OCCC(CCCCC)CCCCC)NC(=O)NCCCN(C)C] | 35 |
| 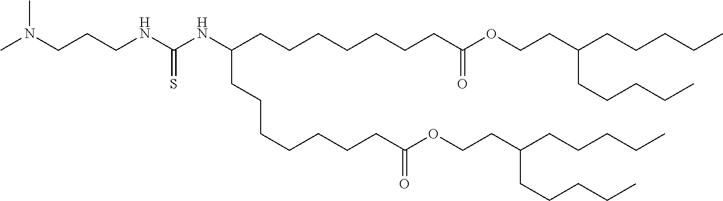 [SMILES: CCCCCC(CCCCC)CCOC(=O)CCCCCCCC(CCCCCCCC(=O)OCCC(CCCCC)CCCCC)NC(=S)NCCCN(C)C] | 36 |
| 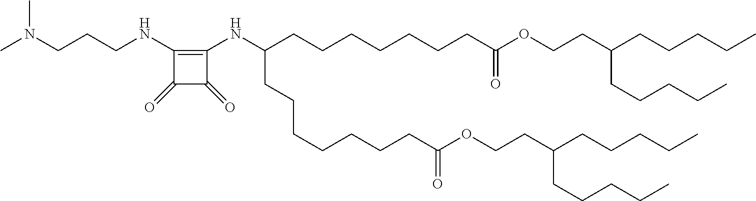 [SMILES: CCCCCC(CCCCC)CCOC(=O)CCCCCCCC(CCCCCCCC(=O)OCCC(CCCCC)CCCCC)Nc1c(NCCCN(C)C)c(=O)c1=O] | 37 |
| 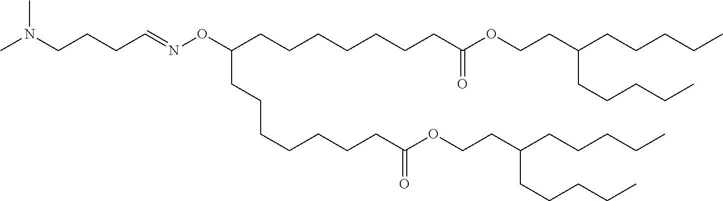 [SMILES: CCCCCC(CCCCC)CCOC(=O)CCCCCCCC(CCCCCCCC(=O)OCCC(CCCCC)CCCCC)O/N=C/CCCN(C)C] | 38 |
| 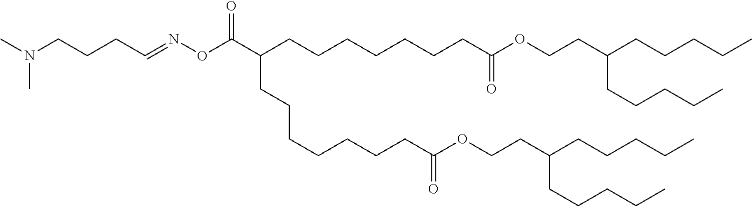 [SMILES: CCCCCC(CCCCC)CCOC(=O)CCCCCCCC(CCCCCCCC(=O)OCCC(CCCCC)CCCCC)C(=O)O/N=C/CCCN(C)C] | 39 |
| 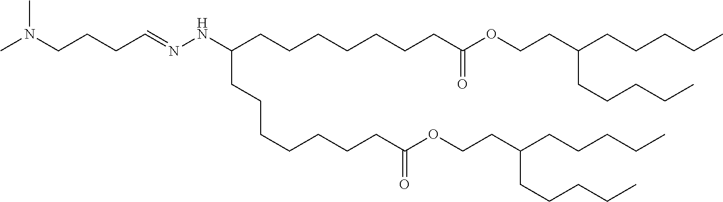 [SMILES: CCCCCC(CCCCC)CCOC(=O)CCCCCCCC(CCCCCCCC(=O)OCCC(CCCCC)CCCCC)N/N=C/CCCN(C)C] | 40 |
| 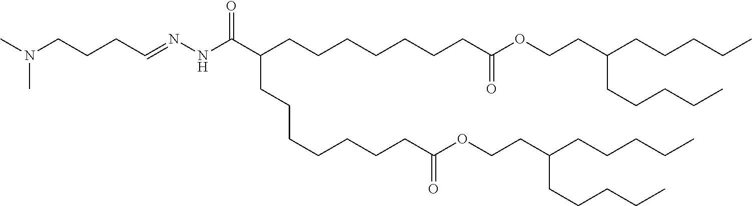 [SMILES: CCCCCC(CCCCC)CCOC(=O)CCCCCCCC(CCCCCCCC(=O)OCCC(CCCCC)CCCCC)C(=O)N/N=C/CCCN(C)C] | 41 |
| 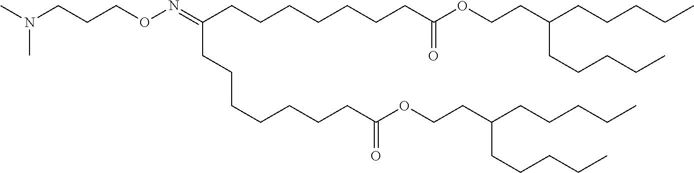 [SMILES: CCCCCC(CCCCC)CCOC(=O)CCCCCCCC(CCCCCCCC(=O)OCCC(CCCCC)CCCCC)=NOCCCN(C)C] | 42 |
| 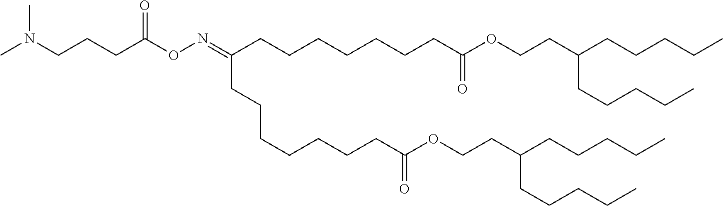 [SMILES: CCCCCC(CCCCC)CCOC(=O)CCCCCCCC(CCCCCCCC(=O)OCCC(CCCCC)CCCCC)=NOC(=O)CCCN(C)C] | 43 |
| 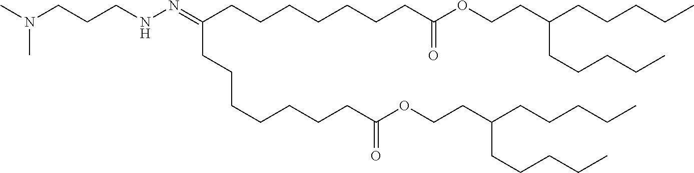 [SMILES: CCCCCC(CCCCC)CCOC(=O)CCCCCCCC(CCCCCCCC(=O)OCCC(CCCCC)CCCCC)=NNCCCN(C)C] | 44 |
| 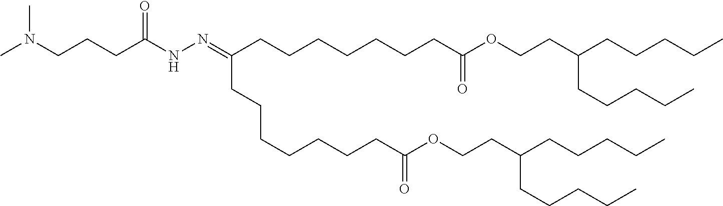 [SMILES: CCCCCC(CCCCC)CCOC(=O)CCCCCCCC(CCCCCCCC(=O)OCCC(CCCCC)CCCCC)=NNC(=O)CCCN(C)C] | 45 |
| 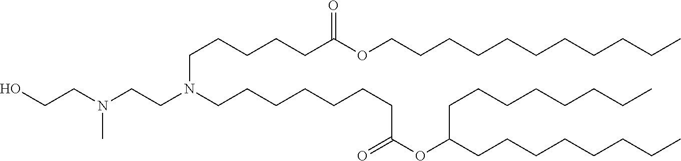 [SMILES: CCCCCCCCCCCOC(=O)CCCCCN(CCCCCCCC(=O)OC(CCCCCCCC)CCCCCCCC)CCN(C)CCO] | 46 |
| 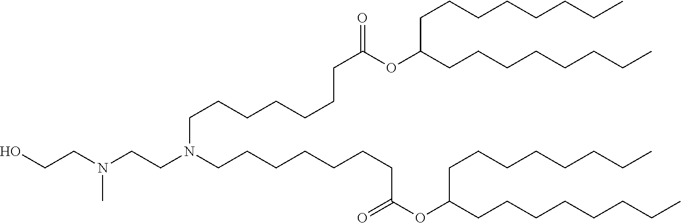 [SMILES: CCCCCCCCC(CCCCCCCC)OC(=O)CCCCCCCN(CCCCCCCC(=O)OC(CCCCCCCC)CCCCCCCC)CCN(C)CCO] | 47 |
| 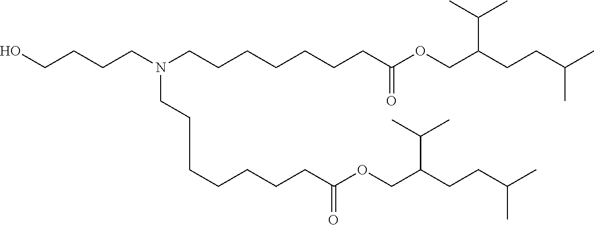 [SMILES: CC(C)CCC(COC(=O)CCCCCCCN(CCCCO)CCCCCCCC(=O)OCC(CCC(C)C)C(C)C)C(C)C] | 48 |
| 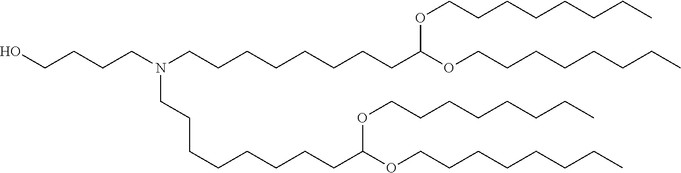 [SMILES: CCCCCCCCOC(CCCCCCCCN(CCCCO)CCCCCCCCC(OCCCCCCCC)OCCCCCCCC)OCCCCCCCC] | 49 |
| 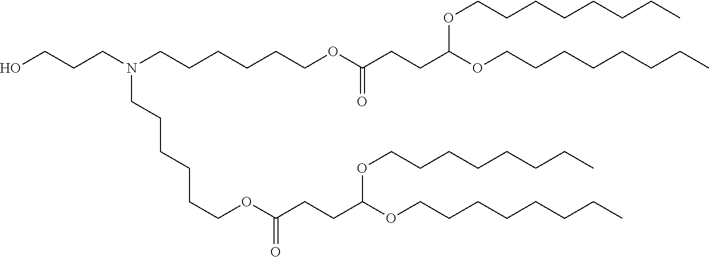 [SMILES: CCCCCCCCOC(CCC(=O)OCCCCCCN(CCCO)CCCCCCOC(=O)CCC(OCCCCCCCC)OCCCCCCCC)OCCCCCCCC] | 50 |
| 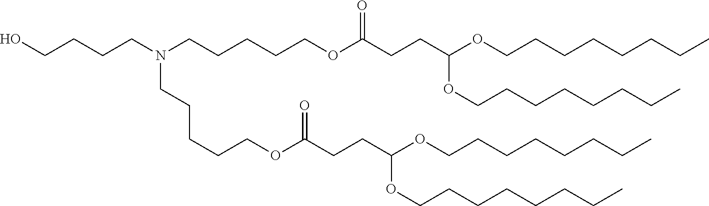 [SMILES: CCCCCCCCOC(CCC(=O)OCCCCCN(CCCCO)CCCCCOC(=O)CCC(OCCCCCCCC)OCCCCCCCC)OCCCCCCCC] | 51 |
| 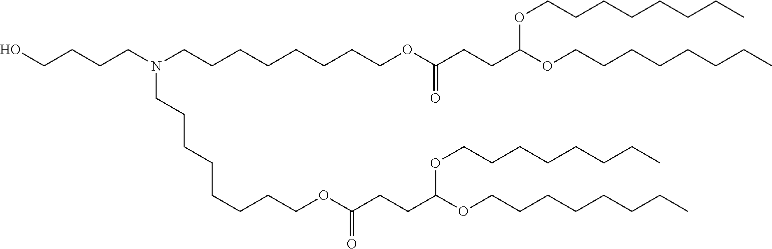 [SMILES: CCCCCCCCOC(CCC(=O)OCCCCCCCCN(CCCCO)CCCCCCCCOC(=O)CCC(OCCCCCCCC)OCCCCCCCC)OCCCCCCCC] | 52 |
| 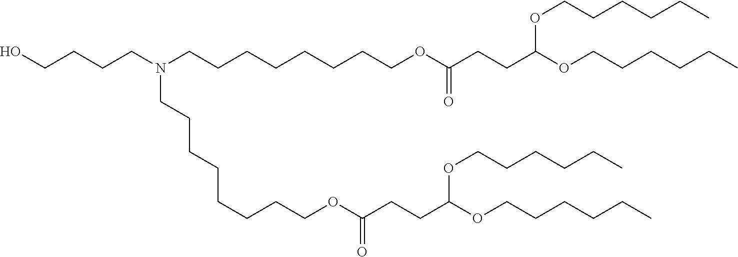 [SMILES: CCCCCCOC(CCC(=O)OCCCCCCCCN(CCCCO)CCCCCCCCOC(=O)CCC(OCCCCCC)OCCCCCC)OCCCCCC] | 53 |
| 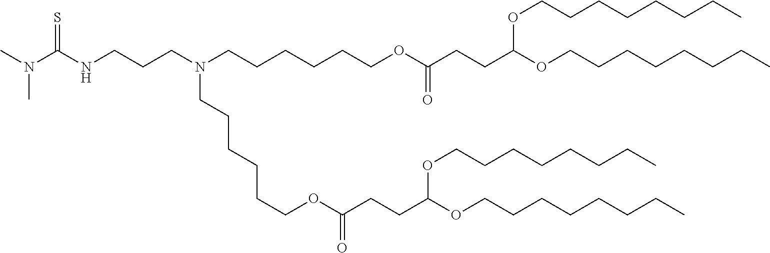 [SMILES: CCCCCCCCOC(CCC(=O)OCCCCCCN(CCCCCCOC(=O)CCC(OCCCCCCCC)OCCCCCCCC)CCCNC(=S)N(C)C)OCCCCCCCC] | 54 |
| 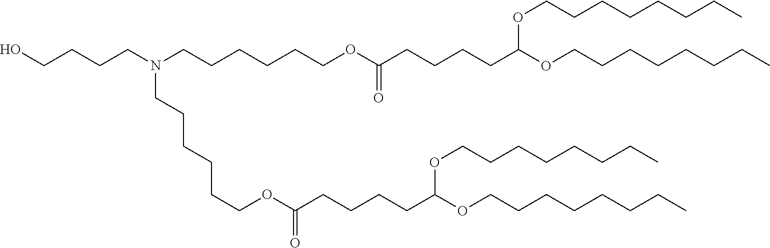 [SMILES: CCCCCCCCOC(CCCCC(=O)OCCCCCCN(CCCCO)CCCCCCOC(=O)CCCCC(OCCCCCCCC)OCCCCCCCC)OCCCCCCCC] | 55 |
| 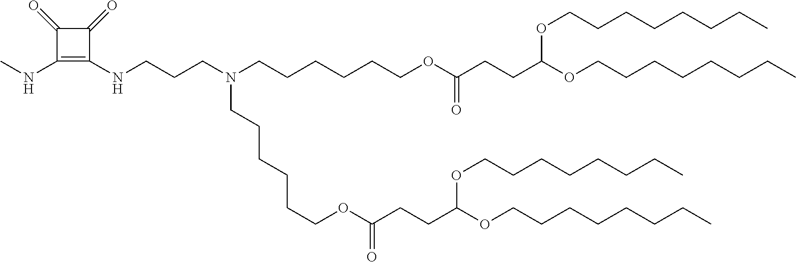 [SMILES: CCCCCCCCOC(CCC(=O)OCCCCCCN(CCCCCCOC(=O)CCC(OCCCCCCCC)OCCCCCCCC)CCCNc1c(NC)c(=O)c1=O)OCCCCCCCC] | 56 |
| 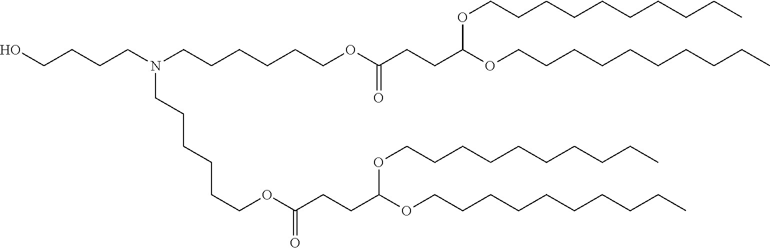 [SMILES: CCCCCCCCCCOC(CCC(=O)OCCCCCCN(CCCCO)CCCCCCOC(=O)CCC(OCCCCCCCCCC)OCCCCCCCCCC)OCCCCCCCCCC] | 57 |
| 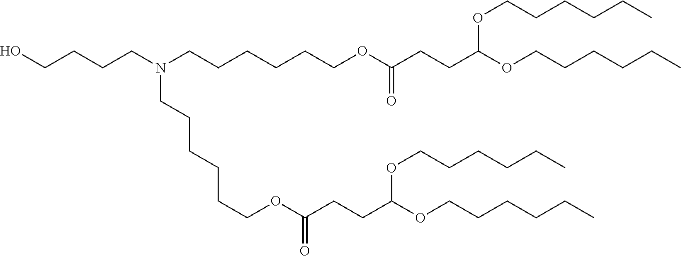 [SMILES: CCCCCCOC(CCC(=O)OCCCCCCN(CCCCO)CCCCCCOC(=O)CCC(OCCCCCC)OCCCCCC)OCCCCCC] | 58 |
| 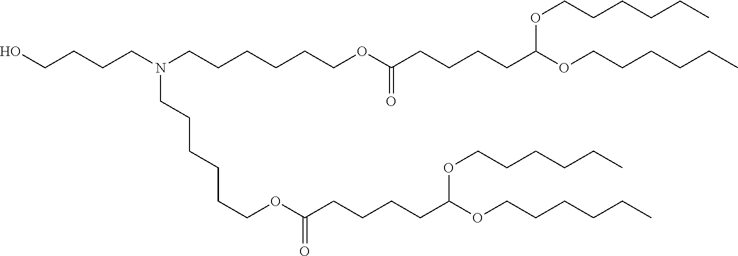 [SMILES: CCCCCCOC(CCCCC(=O)OCCCCCCN(CCCCO)CCCCCCOC(=O)CCCCC(OCCCCCC)OCCCCCC)OCCCCCC] | 59 |
| 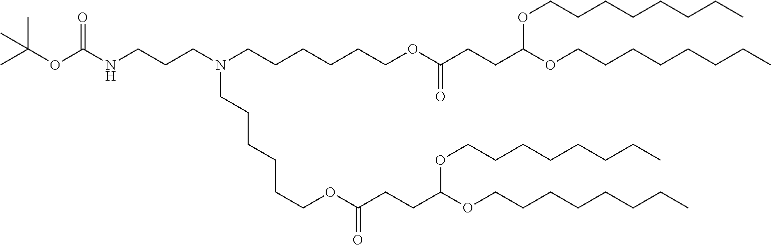 [SMILES: CCCCCCCCOC(CCC(=O)OCCCCCCN(CCCCCCOC(=O)CCC(OCCCCCCCC)OCCCCCCCC)CCCNC(=O)OC(C)(C)C)OCCCCCCCC] | 60 |
| 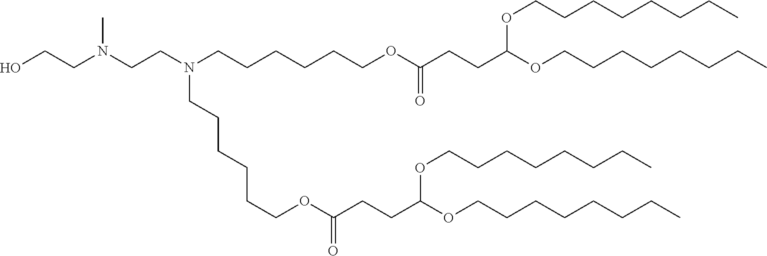 [SMILES: CCCCCCCCOC(CCC(=O)OCCCCCCN(CCCCCCOC(=O)CCC(OCCCCCCCC)OCCCCCCCC)CCN(C)CCO)OCCCCCCCC] | 61 |
| 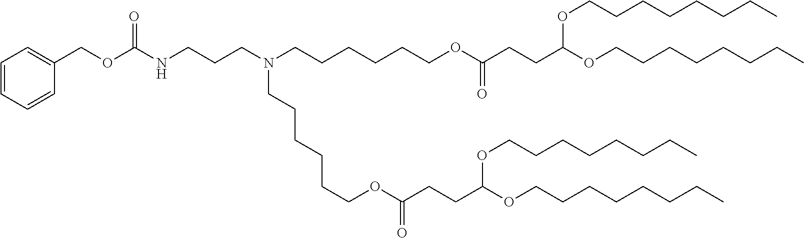 [SMILES: CCCCCCCCOC(CCC(=O)OCCCCCCN(CCCCCCOC(=O)CCC(OCCCCCCCC)OCCCCCCCC)CCCNC(=O)OCc1ccccc1)OCCCCCCCC] | 62 |
| 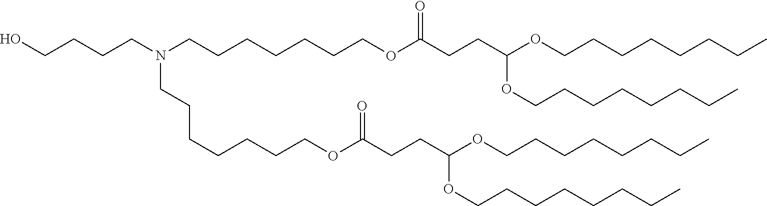 [SMILES: CCCCCCCCOC(CCC(=O)OCCCCCCCN(CCCCO)CCCCCCCOC(=O)CCC(OCCCCCCCC)OCCCCCCCC)OCCCCCCCC] | 64 |
| 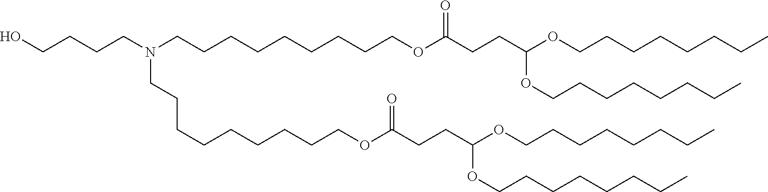 [SMILES: CCCCCCCCOC(CCC(=O)OCCCCCCCCCN(CCCCO)CCCCCCCCCOC(=O)CCC(OCCCCCCCC)OCCCCCCCC)OCCCCCCCC] | 65 |
| 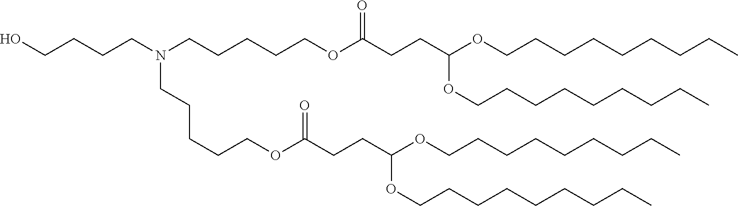 [SMILES: CCCCCCCCCOC(CCC(=O)OCCCCCN(CCCCO)CCCCCOC(=O)CCC(OCCCCCCCCC)OCCCCCCCCC)OCCCCCCCCC] | 66 |
| 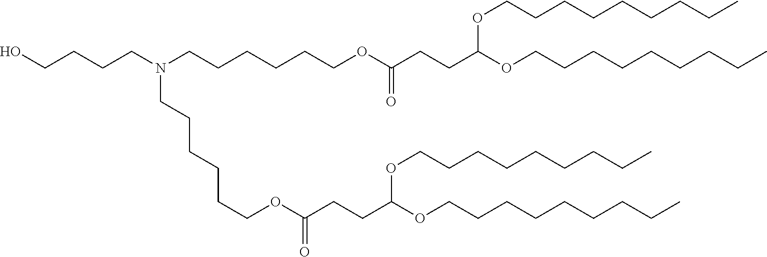 [SMILES: CCCCCCCCCOC(CCC(=O)OCCCCCCN(CCCCO)CCCCCCOC(=O)CCC(OCCCCCCCCC)OCCCCCCCCC)OCCCCCCCCC] | 67 |
| 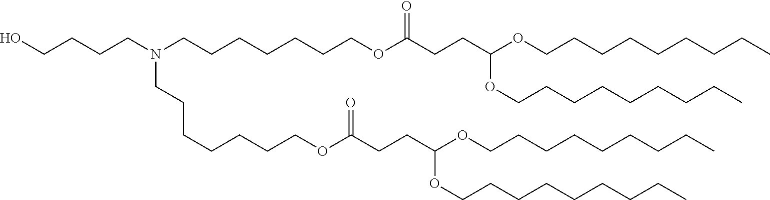 [SMILES: CCCCCCCCCOC(CCC(=O)OCCCCCCCN(CCCCO)CCCCCCCOC(=O)CCC(OCCCCCCCCC)OCCCCCCCCC)OCCCCCCCCC] | 68 |
| 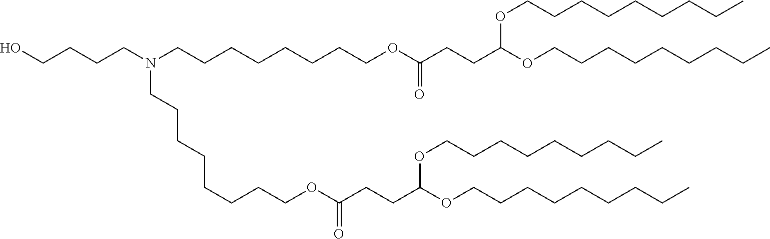 [SMILES: CCCCCCCCCOC(CCC(=O)OCCCCCCCCN(CCCCO)CCCCCCCCOC(=O)CCC(OCCCCCCCCC)OCCCCCCCCC)OCCCCCCCCC] | 69 |
| 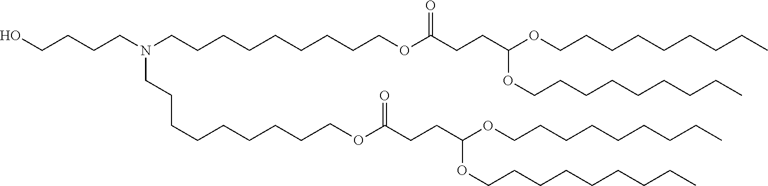 [SMILES: CCCCCCCCCOC(CCC(=O)OCCCCCCCCCN(CCCCO)CCCCCCCCCOC(=O)CCC(OCCCCCCCCC)OCCCCCCCCC)OCCCCCCCCC] | 70 |
| 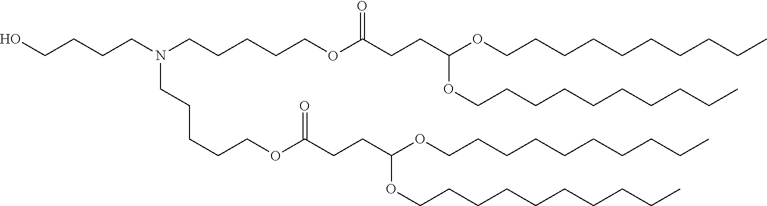 [SMILES: CCCCCCCCCCOC(CCC(=O)OCCCCCN(CCCCO)CCCCCOC(=O)CCC(OCCCCCCCCCC)OCCCCCCCCCC)OCCCCCCCCCC] | 71 |
| 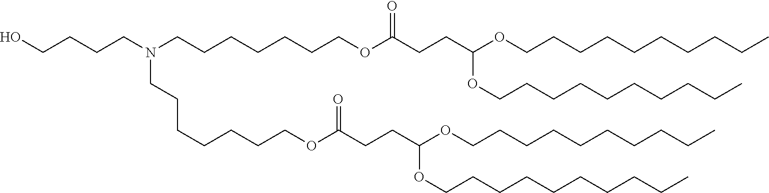 [SMILES: CCCCCCCCCCOC(CCC(=O)OCCCCCCCN(CCCCO)CCCCCCCOC(=O)CCC(OCCCCCCCCCC)OCCCCCCCCCC)OCCCCCCCCCC] | 72 |
| 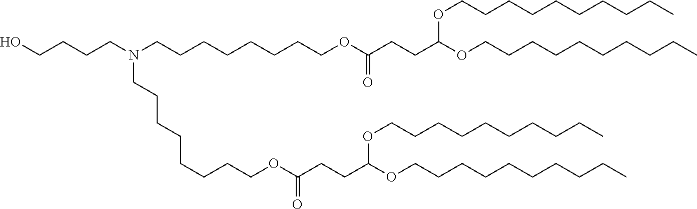 [SMILES: CCCCCCCCCCOC(CCC(=O)OCCCCCCCCN(CCCCO)CCCCCCCCOC(=O)CCC(OCCCCCCCCCC)OCCCCCCCCCC)OCCCCCCCCCC] | 73 |
| 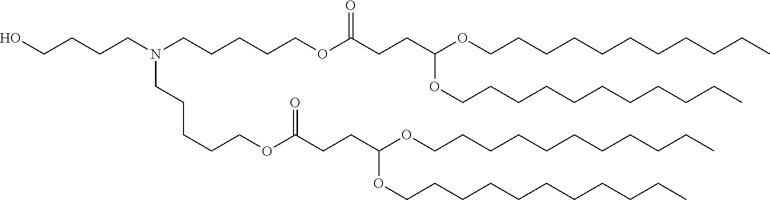 [SMILES: CCCCCCCCCCCOC(CCC(=O)OCCCCCN(CCCCO)CCCCCOC(=O)CCC(OCCCCCCCCCCC)OCCCCCCCCCCC)OCCCCCCCCCCC] | 74 |
| 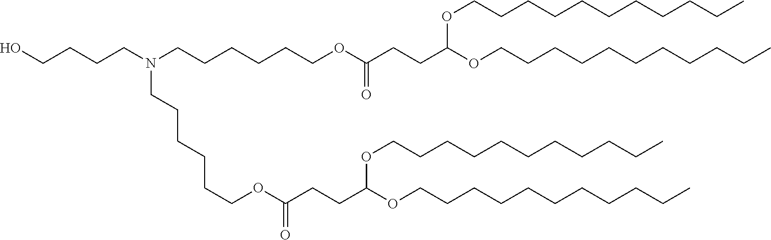 [SMILES: CCCCCCCCCCCOC(CCC(=O)OCCCCCCN(CCCCO)CCCCCCOC(=O)CCC(OCCCCCCCCCCC)OCCCCCCCCCCC)OCCCCCCCCCCC] | 75 |
| 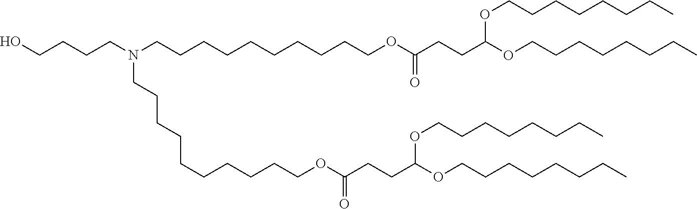 [SMILES: CCCCCCCCOC(CCC(=O)OCCCCCCCCCCN(CCCCO)CCCCCCCCCCOC(=O)CCC(OCCCCCCCC)OCCCCCCCC)OCCCCCCCC] | 76 |
| 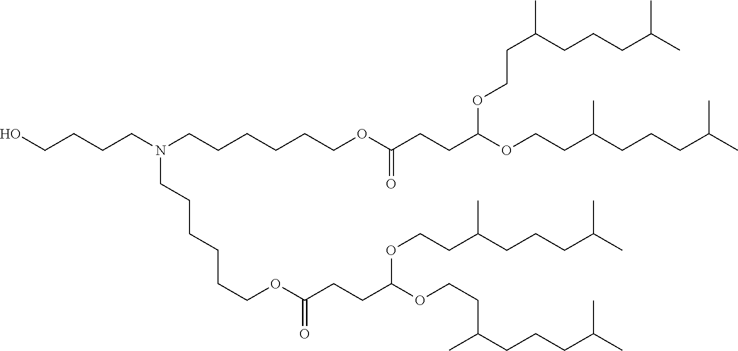 [SMILES: CC(C)CCCC(C)CCOC(CCC(=O)OCCCCCCN(CCCCO)CCCCCCOC(=O)CCC(OCCC(C)CCCC(C)C)OCCC(C)CCCC(C)C)OCCC(C)CCCC(C)C] | 77 |
| 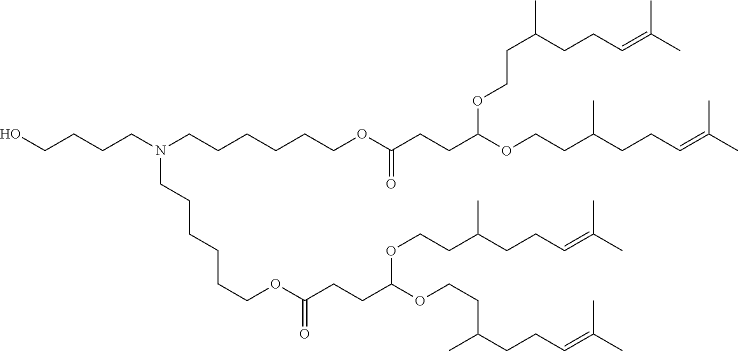 [SMILES: CC(C)=CCCC(C)CCOC(CCC(=O)OCCCCCCN(CCCCO)CCCCCCOC(=O)CCC(OCCC(C)CCC=C(C)C)OCCC(C)CCC=C(C)C)OCCC(C)CCC=C(C)C] | 78 |
| 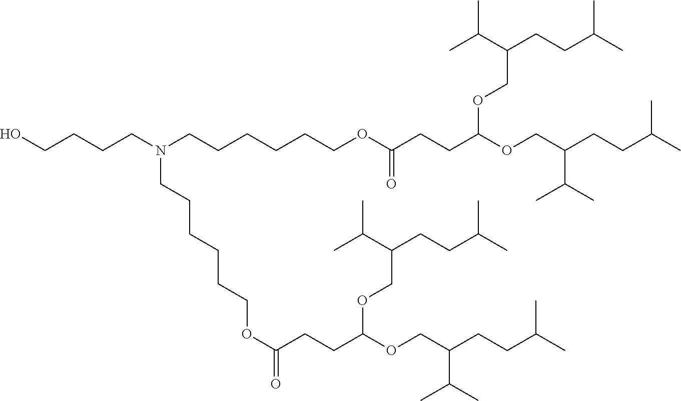 [SMILES: CC(C)CCC(COC(CCC(=O)OCCCCCCN(CCCCO)CCCCCCOC(=O)CCC(OCC(CCC(C)C)C(C)C)OCC(CCC(C)C)C(C)C)OCC(CCC(C)C)C(C)C)C(C)C] | 79 |
| 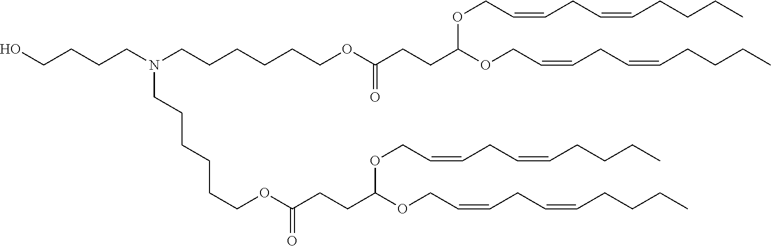 [SMILES: CCCC/C=C\C/C=C\COC(CCC(=O)OCCCCCCN(CCCCO)CCCCCCOC(=O)CCC(OC/C=C\C/C=C\CCCC)OC/C=C\C/C=C\CCCC)OC/C=C\C/C=C\CCCC] | 80 |
| 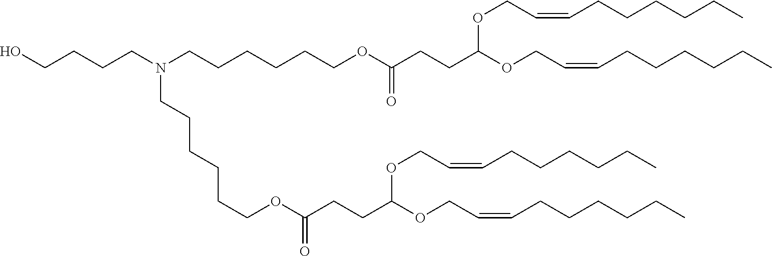 [SMILES: CCCCCC/C=C\COC(CCC(=O)OCCCCCCN(CCCCO)CCCCCCOC(=O)CCC(OC/C=C\CCCCCC)OC/C=C\CCCCCC)OC/C=C\CCCCCC] | 81 |
| 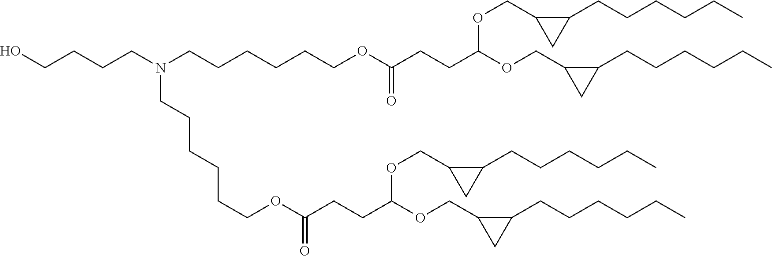 [SMILES: CCCCCCC1CC1COC(CCC(=O)OCCCCCCN(CCCCO)CCCCCCOC(=O)CCC(OCC1CC1CCCCCC)OCC1CC1CCCCCC)OCC1CC1CCCCCC] | 82 |
| 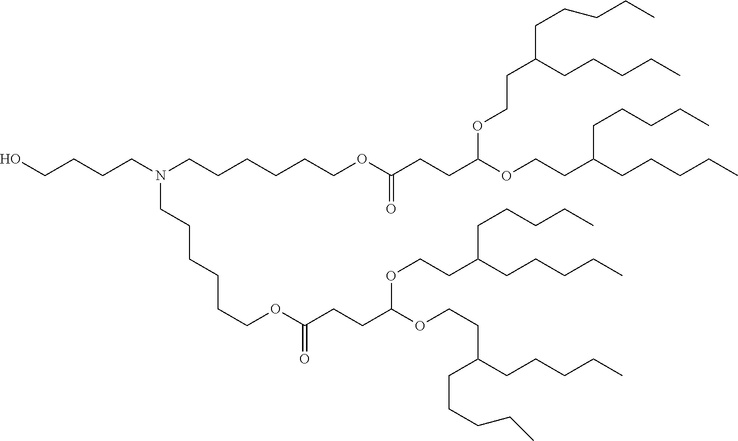 [SMILES: CCCCCC(CCCCC)CCOC(CCC(=O)OCCCCCCN(CCCCO)CCCCCCOC(=O)CCC(OCCC(CCCCC)CCCCC)OCCC(CCCCC)CCCCC)OCCC(CCCCC)CCCCC] | 83 |
| 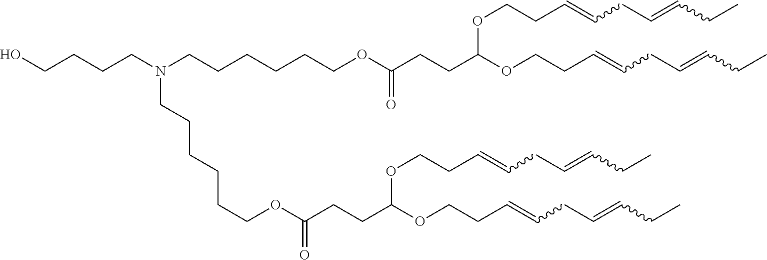 [SMILES: CCC=CCC=CCCOC(CCC(=O)OCCCCCCN(CCCCO)CCCCCCOC(=O)CCC(OCCC=CCC=CCC)OCCC=CCC=CCC)OCCC=CCC=CCC] | 84 |
| 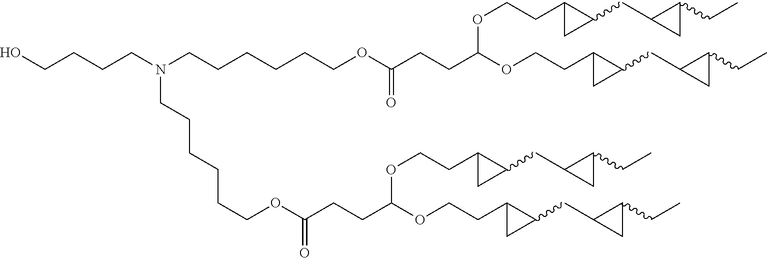 [SMILES: CCC1CC1CC1CC1CCOC(CCC(=O)OCCCCCCN(CCCCO)CCCCCCOC(=O)CCC(OCCC1CC1CC1CC1CC)OCCC1CC1CC1CC1CC)OCCC1CC1CC1CC1CC] | 85 |
| 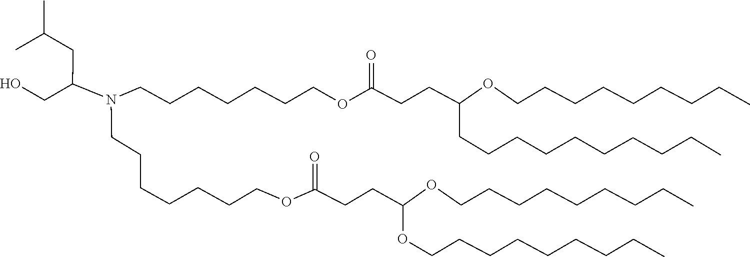 [SMILES: CCCCCCCCCCC(CCC(=O)OCCCCCCCN(CCCCCCCOC(=O)CCC(OCCCCCCCCC)OCCCCCCCCC)C(CO)CC(C)C)OCCCCCCCCC] | 86 |
| 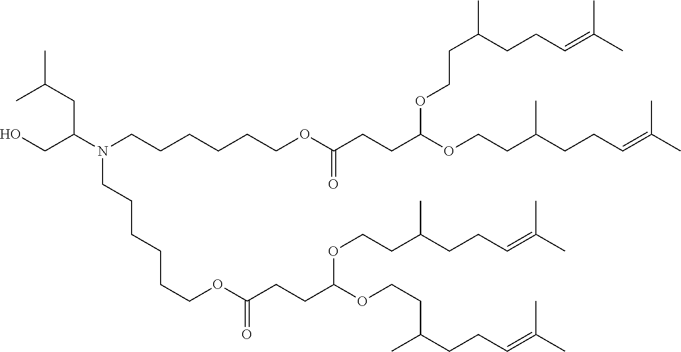 [SMILES: CC(C)=CCCC(C)CCOC(CCC(=O)OCCCCCCN(CCCCCCOC(=O)CCC(OCCC(C)CCC=C(C)C)OCCC(C)CCC=C(C)C)C(CO)CC(C)C)OCCC(C)CCC=C(C)C] | 87 |
| 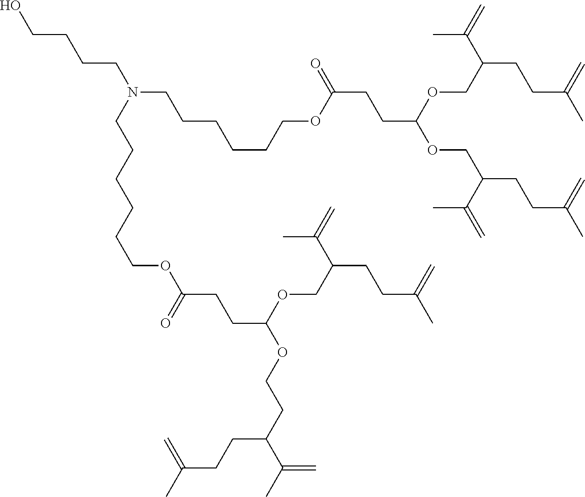 [SMILES: C=C(C)CCC(CCOC(CCC(=O)OCCCCCCN(CCCCO)CCCCCCOC(=O)CCC(OCC(CCC(=C)C)C(=C)C)OCC(CCC(=C)C)C(=C)C)OCC(CCC(=C)C)C(=C)C)C(=C)C] | 88 |
| 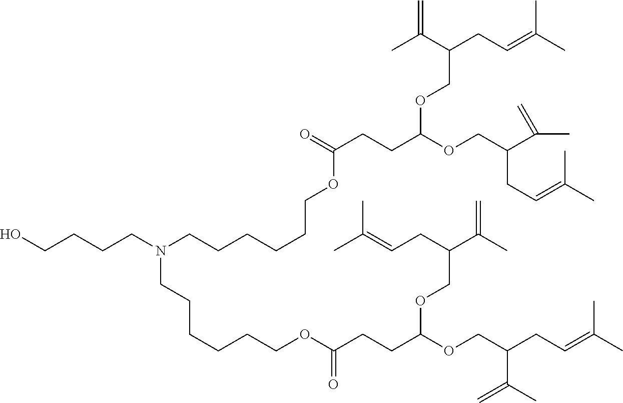 [SMILES: C=C(C)C(CC=C(C)C)COC(CCC(=O)OCCCCCCN(CCCCO)CCCCCCOC(=O)CCC(OCC(CC=C(C)C)C(=C)C)OCC(CC=C(C)C)C(=C)C)OCC(CC=C(C)C)C(=C)C] | 89 |

In some embodiments, an LNP of the present disclosure comprises an ionizable lipid disclosed in PCT Application Publication WO2023044333A1, which is incorporated by reference herein, in its entirety.

### Formula (CY)

In some embodiments, an LNP disclosed herein comprises an ionizable lipid of Formula (CY)

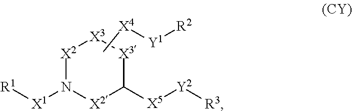

or a pharmaceutically acceptable salt thereof,
wherein:
R¹ is selected from the group consisting of —OH, —OAc, R^{1a}

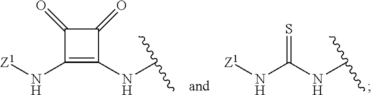

Z¹ is optionally substituted C₁-C₆ alkyl;
X¹ is optionally substituted C₂-C₆ alkylenyl;
X² is selected from the group consisting of a bond, —CH₂— and —CH₂CH₂—;
X²′ is selected from the group consisting of a bond, —CH₂— and —CH₂CH₂—;
X³ is selected from the group consisting of a bond, —CH₂— and —CH₂CH₂—;
X³′ is selected from the group consisting of a bond, —CH₂— and —CH₂CH₂—;
X⁴ and X³ are independently optionally substituted C₂-C₁₄ alkylenyl or optionally substituted C₂-C₁₄ alkenylenyl;
Y¹ and Y² are independently selected from the group consisting of

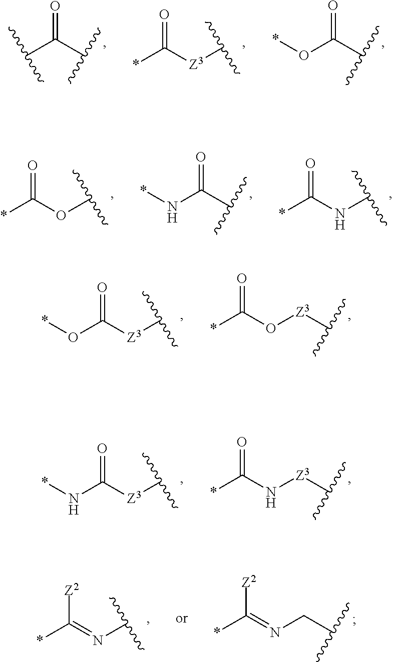

wherein the bond marked with an “*” is attached to X⁴ or X⁵;
each Z² is independently H or optionally substituted C₁-C₅ alkyl;
each Z³ is independently optionally substituted C₁-C₆ alkylenyl;
R² is selected from the group consisting of optionally substituted C₄-C₂₀ alkyl, optionally substituted C₂-C₁₄ alkenyl, and —(CH₂)ₚCH(OR⁶)(OR⁷);
R³ is selected from the group consisting of optionally substituted C₄-C₂₀ alkyl, optionally substituted C₂-C₁₄ alkenyl, or —(CH₂)ₚCH(OR⁸)(OR³);
R^{1a} is:

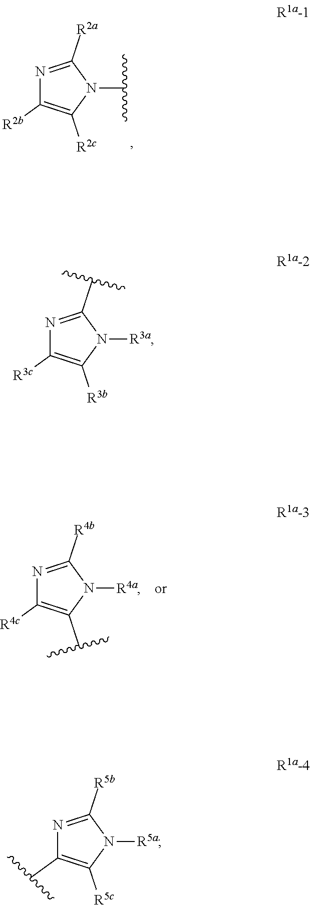

R^{2a}, R²⁰, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl;
R^{5a}, R^{5b}, and R^{5c} are independently hydrogen and C₁-C₆ alkyl;
R⁶, R⁷, R⁸, and R⁹ are independently optionally substituted C₁-C₁₄ alkyl, optionally substituted C₂-C₁₄ alkenyl, or —(CH₂)ₘ-A-(CH₂)ₙH;
each A is independently a C₃-C₈ cycloalkylenyl;
each m is independently 0, 1, 2, 3, 4, 5, 6, 7, 8, 9, 10, 11, or 12;
each n is independently 0, 1, 2, 3, 4, 5, 6, 7, 8, 9, 10, 11, or 12;
p is selected from the group consisting of 0, 1, 2, 3, 4, 5, 6, and 7; and
q is selected from the group consisting of 0, 1, 2, 3, 4, 5, 6, and 7.

### Formulas (CY-I), (CY-II), (CY-III), (CY-IV), and (CY-V)

In some embodiments, the present disclosure includes a compound of Formula (CY-I), (CY-II), (CY-III), (CY-IV), or (CY-V):

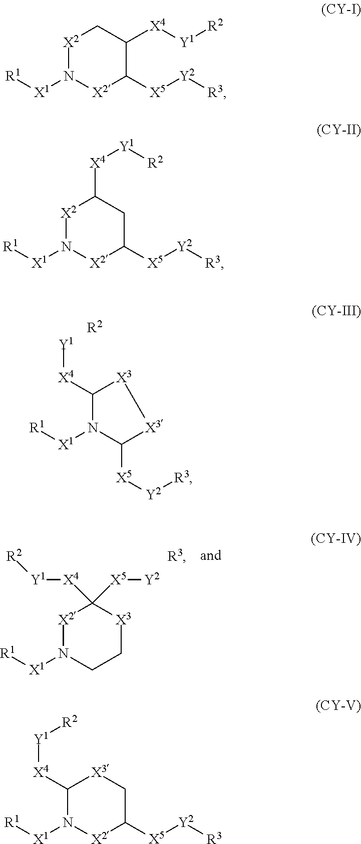

or a pharmaceutically acceptable salt thereof,
wherein X¹, X², X²′, X³, X³′, X⁴, X⁵, Y¹, Y², R¹, R², and R³ are defined herein.

### Formulas (CY-VI) and (CY-VII)

In some embodiments, the present disclosure includes a compound of Formula (CY-VI) or (CY-VII):

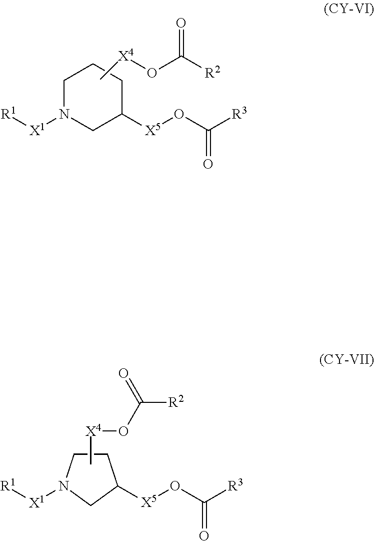

or a pharmaceutically acceptable salt thereof,
wherein X¹, X⁴, X⁵, R¹, R², and R³ are defined herein.

### Formulas (CY-VIII) and (CY-IX)

In some embodiments, the present disclosure includes a compound of Formula (CY-VIII) or (CY-IX):

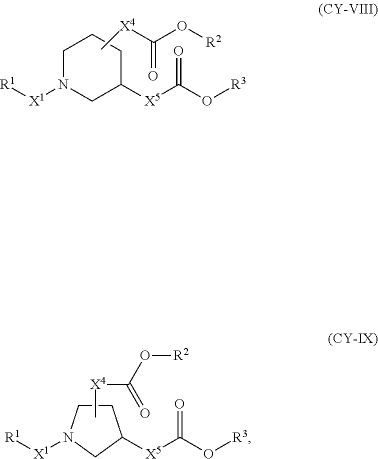

or pharmaceutically acceptable salt thereof,
wherein X¹, X⁴, X⁵, R¹, R², and R³ are defined herein.

### Formulas (CY-IV-a), (CY-IV-b), and (CY-IV-c)

In some embodiments, the present disclosure includes a compound of Formula (CY-IV-a), (CY-IV-b), or (CY-IV-c)

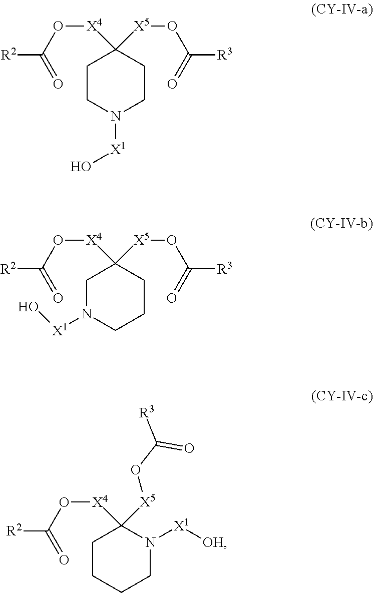

or pharmaceutically acceptable salt thereof.
wherein X¹, X⁴, X⁵, R², and R³ are defined herein.

### Formulas (CY-IV-d), (CY-IV-e), and (CY-IV-f)

In some embodiments, the present disclosure includes a compound of Formula (CY-IV-d), (CY-IV-e), or (CY-IV-f)

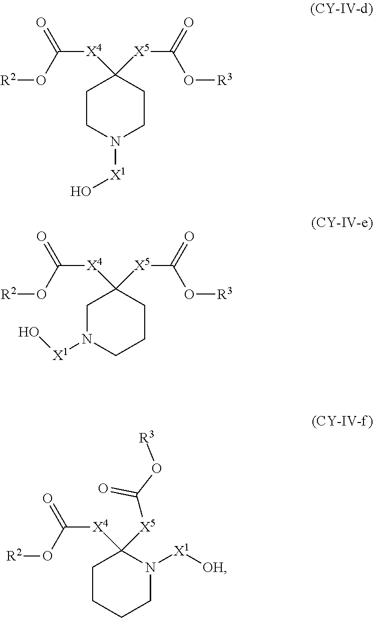

or pharmaceutically acceptable salt thereof.
wherein X¹, X⁴, X⁵, R², and R³ are defined herein.

### Formula (CY-IV)

In some embodiments, Lipids of the Disclosure have a structure of Formula (CY-IV′):

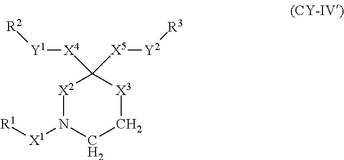

or a pharmaceutically acceptable salt thereof, wherein R¹, R², R³, X¹, X², X³, X⁴, X⁵, Y¹, and
Y² are as defined in connection with Formula (CY-I′).

In some embodiments, Lipids of the Disclosure have a structure of Formula (CY-IV′), wherein:
R¹ is —OH, R^{1a},

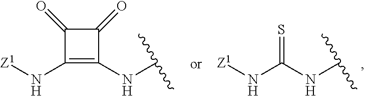

wherein Z¹ is optionally substituted C₁-C₆ alkyl;
X¹ is optionally substituted C₂-C₆ alkylenyl;
X² and X³ are independently a bond, —CH₂—, or —CH₂CH₂—;
X⁴ and X⁵ are independently optionally substituted C₂-C₁₄ alkylenyl;
Y¹ and Y² are independently

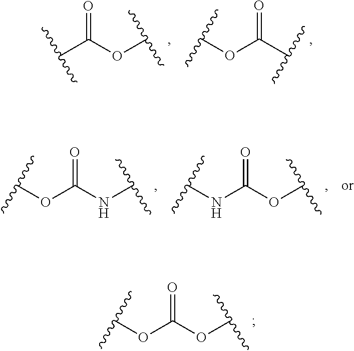 [SMILES: *OC(*)=O ; *OC(*)=O ; *NC(=O)O* ; *NC(=O)O* ; *OC(=O)O*]

R² and R³ are independently optionally substituted C₄-C₂₀ alkyl;
R^{1a} is:

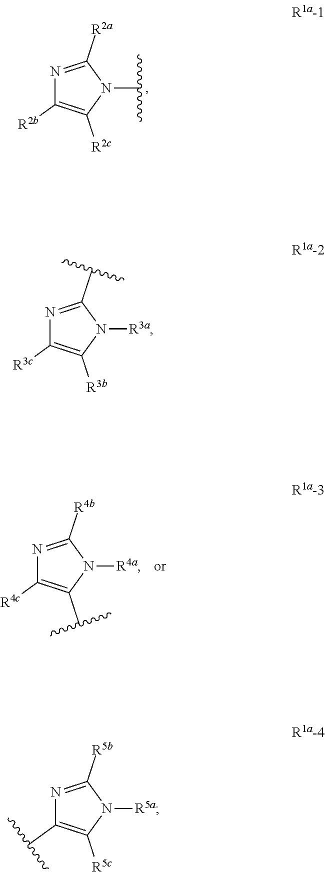

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl; and
R^{5a}, R^{5b}, and R^{5c} are independently hydrogen and C₁-C₆ alkyl

In some embodiments, Lipids of the Disclosure have a structure of Formula (CY-IV′), wherein R¹ is —OH,

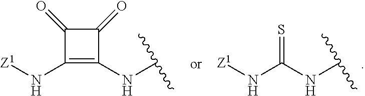

In some embodiments, Lipids of the Disclosure have a structure of Formula (CY-IV′), wherein Y¹ and Y² are independently:

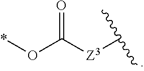

In some embodiments, Lipids of the Disclosure have a structure of Formula (CY-IV′), wherein R² is —CH(OR⁶)(OR⁷).

In some embodiments, Lipids of the Disclosure have a structure of Formula (CY-IV′), wherein R³ is —CH(OR⁸)(OR⁹).

Non-limiting examples of lipids having a structure of Formula (CY-IV′) include compounds CY7, CY8, CY19, CY20, CY21, CY28, CY29, CY40, CY41, CY42, CY48, CY49, CY58, CY59, and CY60.

### Formula (CY-VI′)

In some embodiments, Lipids of the Disclosure have a structure of Formula (CY-VI′):

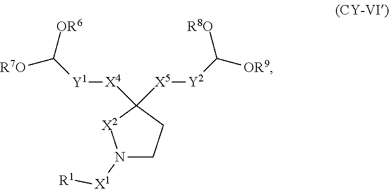

or a pharmaceutically acceptable salt thereof, wherein R¹, R⁶, R⁷, R³, R³, X¹, X², X³, X⁴, X⁵, Y¹, and Y² are as defined in connection with Formula (CY-I′).

In some embodiments, Lipids of the Disclosure have a structure of Formula (CY-VI′), or a pharmaceutically acceptable salt thereof, wherein R¹ is —OH.

In some embodiments, Lipids of the Disclosure have a structure of Formula (CY-VI′), or a pharmaceutically acceptable salt thereof, wherein X¹ is C₂-C₆ alkylenyl.

In some embodiments, Lipids of the Disclosure have a structure of Formula (CY-VI′), or a pharmaceutically acceptable salt thereof, wherein X² is —CH₂CH₂—.

In some embodiments, Lipids of the Disclosure have a structure of Formula (CY-VI′), or a pharmaceutically acceptable salt thereof, wherein X⁴ is C₂-C₆ alkylenyl.

In some embodiments, Lipids of the Disclosure have a structure of Formula (CY-VI′), or a pharmaceutically acceptable salt thereof, wherein X⁵ is C₂-C₆ alkylenyl.

In some embodiments, Lipids of the Disclosure have a structure of Formula (CY-VI′), or a pharmaceutically acceptable salt thereof, wherein Y¹ is:

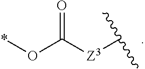

In some embodiments, Lipids of the Disclosure have a structure of Formula (CY-VI′), or a pharmaceutically acceptable salt thereof, wherein Y² is:

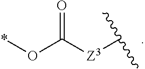

In some embodiments, Lipids of the Disclosure have a structure of Formula (CY-VI′), or a pharmaceutically acceptable salt thereof, wherein each Z³ is independently optionally substituted C₁-C₆ alkylenyl.

In some embodiments, Lipids of the Disclosure have a structure of Formula (CY-VI′), or a pharmaceutically acceptable salt thereof, wherein each Z³ is —CH₂CH₂—.

In some embodiments, Lipids of the Disclosure have a structure of Formula (CY-VI′), or a pharmaceutically acceptable salt thereof, wherein R⁶ is C₅-C₁₄ alkyl.

In some embodiments, Lipids of the Disclosure have a structure of Formula (CY-VI′), or a pharmaceutically acceptable salt thereof, wherein R⁷ is C₅-C₁₄ alkyl.

In some embodiments, Lipids of the Disclosure have a structure of Formula (CY-VI′), or a pharmaceutically acceptable salt thereof, wherein R⁶ is C₆-C₁₄ alkenyl.

In some embodiments, Lipids of the Disclosure have a structure of Formula (CY-VI′), or a pharmaceutically acceptable salt thereof, wherein R⁷ is C₆-C₁₄ alkenyl.

In some embodiments, Lipids of the Disclosure have a structure of Formula (CY-VI′), or a pharmaceutically acceptable salt thereof, wherein R⁸ is C₅-C₁₆ alkyl.

In some embodiments, Lipids of the Disclosure have a structure of Formula (CY-VI′), or a pharmaceutically acceptable salt thereof, wherein R⁹ is C₅-C₁₄ alkyl.

In some embodiments, Lipids of the Disclosure have a structure of Formula (CY-VI′), or a pharmaceutically acceptable salt thereof, wherein R⁸ is C₆-C₁₄ alkenyl.

In some embodiments, Lipids of the Disclosure have a structure of Formula (CY-VI′), or a pharmaceutically acceptable salt thereof, wherein R⁹ is C₆-C₁₄ alkenyl.

In some embodiments, Lipids of the Disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof. In some embodiments, Lipids of the Disclosure are selected from any lipid in Table (II) below or a pharmaceutically acceptable salt thereof: R¹

In some embodiments, R¹ is selected from the group consisting of —OH, —OAc, R^{1a}

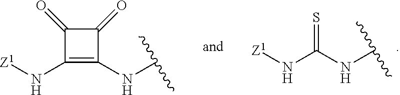

In some embodiments, R¹ is —OH or —OAc. In some embodiments, R¹ is OH. In some embodiments, R¹ is —OAc. In some embodiments, R¹ is R^{1a} In some embodiments, R¹ is imidazolyl. In some embodiments, R¹ is

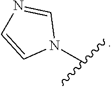 [SMILES: *n1ccnc1]

### R²

In some embodiments, R² is selected from the group consisting of optionally substituted C₄-C₂₀ alkyl, optionally substituted C₂-C₁₄ alkenyl, and —(CH₂)pCH(OR⁶)(OR⁷).

In some embodiments, R² is optionally substituted C₄-C₂₀ alkyl. In some embodiments, R² is optionally substituted C₈-C₁₇ alkyl. In some embodiments, R² is optionally substituted C₉-C₁₆ alkyl. In some embodiments, R² is optionally substituted C₈-C₁₀ alkyl. In some embodiments, R² is optionally substituted C₁₁-C₁₃ alkyl. In some embodiments, R² is optionally substituted C₁₄-C₁₆ alkyl. In some embodiments, R² is optionally substituted C₉ alkyl. In some embodiments, R² is optionally substituted C₁₀ alkyl. In some embodiments, R² is optionally substituted C₁₁ alkyl. In some embodiments, R² is optionally substituted C₁₂ alkyl. In some embodiments, R² is optionally substituted C₁₃ alkyl. In some embodiments, R² is optionally substituted C₁₄ alkyl. In some embodiments, R² is optionally substituted C₁₅ alkyl. In some embodiments, R² is optionally substituted C₁₆ alkyl.

In some embodiments, R² is optionally substituted C₂-C₁₄ alkenyl. In some embodiments, R² is optionally substituted C₅-C₁₄ alkenyl. In some embodiments, R² is optionally substituted C₇-C₁₄ alkenyl. In some embodiments, R² is optionally substituted C₉-C₁₄ alkenyl. In some embodiments, R² is optionally substituted C₁₀-C₁₄ alkenyl. In some embodiments, R² is optionally substituted C₁₂-C₁₄ alkenyl.

In some embodiments, R² is —(CH₂)ₚCH(OR⁶)(OR⁷). In some embodiments, R² is —CH(OR⁶)(OR⁷). In some embodiments, R² is —CH₂CH(OR⁶)(OR⁷). In some embodiments, R² is —(CH₂)₂CH(OR⁶)(OR⁷). In some embodiments, R² is —(CH₂)₃CH(OR⁶)(OR⁷). In some embodiments, R² is —(CH₂)₄CH(OR⁶)(OR⁷).

In some embodiments, R² is selected from the group consisting of

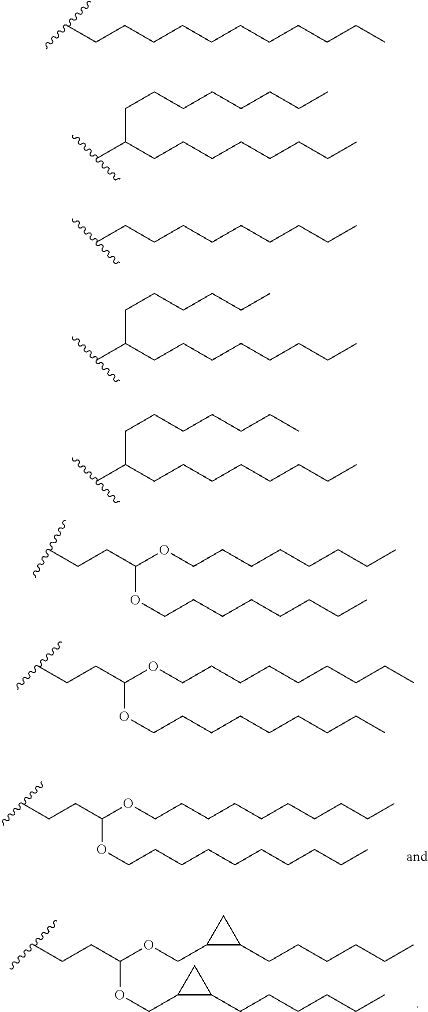

### R³

In some embodiments, R³ is selected from the group consisting of optionally substituted C₄-C₂₀ alkyl, optionally substituted C₂-C₁₄ alkenyl, and —(CH₂)_{q}CH(OR⁶)(OR⁷).

In some embodiments, R³ is optionally substituted C₄-C₂₀ alkyl. In some embodiments, R³ is optionally substituted C₈-C₁₇ alkyl. In some embodiments, R³ is optionally substituted C₉-C₁₆ alkyl. In some embodiments, R³ is optionally substituted C₈-C₁₀ alkyl. In some embodiments, R³ is optionally substituted C₁₁-C₁₃ alkyl. In some embodiments, R³ is optionally substituted C₁₄-C₁₆ alkyl. In some embodiments, R³ is optionally substituted C₉ alkyl. In some embodiments, R³ is optionally substituted C₁₀ alkyl. In some embodiments, R³ is optionally substituted C₁₁ alkyl. In some embodiments, R³ is optionally substituted C₁₂ alkyl. In some embodiments, R³ is optionally substituted C₁₃ alkyl. In some embodiments, R³ is optionally substituted C₁₄ alkyl. In some embodiments, R³ is optionally substituted C₁₅ alkyl. In some embodiments, R³ is optionally substituted C₁₆ alkyl.

In some embodiments, R³ is optionally substituted C₂-C₁₄ alkenyl. In some embodiments, R³ is optionally substituted C₅-C₁₄ alkenyl. In some embodiments, R³ is optionally substituted C₇-C₁₄ alkenyl. In some embodiments, R³ is optionally substituted C₉-C₁₄ alkenyl. In some embodiments, R³ is optionally substituted C₁₀-C₁₄ alkenyl. In some embodiments, R³ is optionally substituted C₁₂-C₁₄ alkenyl.

In some embodiments, R³ is —(CH₂)_{q}CH(OR⁸)(OR⁹). In some embodiments, R³ is —CH(OR⁸)(OR⁹). In some embodiments, R³ is —CH₂CH(OR⁸)(OR⁹). In some embodiments, R³ is —(CH₂)₂CH(OR⁸)(OR⁹). In some embodiments, R³ is —(CH₂)₃CH(OR⁸)(OR⁹). In some embodiments, R³ is —(CH₂)₄CH(OR⁸)(OR⁹).

In some embodiments, R³ is selected from the group consisting of

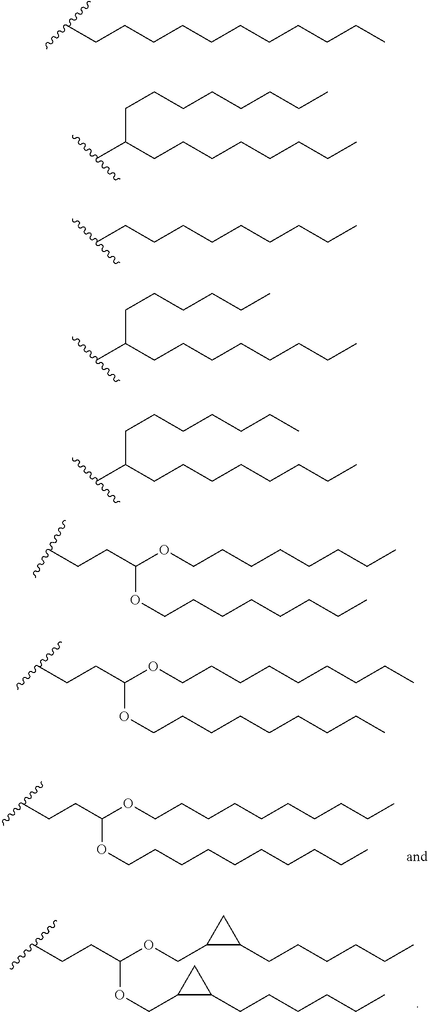

R⁶, R⁷, R⁸, R⁹

In some embodiments, R⁶, R⁷, R⁸, and R⁹ are independently optionally substituted C₁-C₁₄ alkyl, optionally substituted C₂-C₁₄ alkenyl, or —(CH₂)ₘ-A-(CH₂)ₙH. In some embodiments, R⁶, R⁷, R⁸, and R⁹ are independently optionally substituted C₁-C₁₄ alkyl. In some embodiments, R⁶, R⁷, R⁸, and R⁹ are independently optionally substituted C₂-C₁₄ alkenyl. In some embodiments, R⁶, R⁷, R⁸, and R⁹ are independently-(CH₂)ₘ-A-(CH₂)ₙH.

In some embodiments, R⁶ is optionally substituted C₁-C₁₄ alkyl, optionally substituted C₂-C₁₄ alkenyl, or —(CH₂)ₘ-A-(CH₂)ₙH. In some embodiments, R⁶ is optionally substituted C₃-C₁₀ alkyl. In some embodiments, R⁶ is optionally substituted C₄-C₁₀ alkyl. In some embodiments, R⁶ is independently optionally substituted C₅-C₁₀ alkyl. In some embodiments, R⁶ is optionally substituted C₉-C₁₀ alkyl. In some embodiments, R⁶ is optionally substituted C₁-C₁₄ alkyl. In some embodiments, R⁶ is optionally substituted C₂-C₁₄ alkenyl. In some embodiments, R⁶ is —(CH₂)ₘ-A-(CH₂)ₙH.

In some embodiments, R⁷ is optionally substituted C₁-C₁₄ alkyl, optionally substituted C₂-C₁₄ alkenyl, or —(CH₂)ₘ-A-(CH₂)ₙH. In some embodiments, R⁷ is optionally substituted C₃-C₁₀ alkyl. In some embodiments, R⁷ is optionally substituted C₄-C₁₀ alkyl. In some embodiments, R⁷ is optionally substituted C₅-C₁₀ alkyl. In some embodiments, R⁷ is optionally substituted C₉-C₁₀ alkyl. In some embodiments, R⁷ is optionally substituted C₁-C₁₄ alkyl. In some embodiments, R⁷ is optionally substituted optionally substituted C₂-C₁₄ alkenyl. In some embodiments, R⁷ is —(CH₂)ₘ-A-(CH₂)ₙH.

In some embodiments, R⁸ is optionally substituted C₁-C₁₄ alkyl, optionally substituted C₂-C₁₄ alkenyl, or —(CH₂)ₘ-A-(CH₂)ₙH. In some embodiments, R⁸ is optionally substituted C₃-C₁₀ alkyl. In some embodiments, R⁸ is optionally substituted C₄-C₁₀ alkyl. In some embodiments, R⁸ is optionally substituted C₅-C₁₀ alkyl. In some embodiments, R⁸ is optionally substituted C₉-C₁₀ alkyl. In some embodiments, R⁸ is optionally substituted C₁-C₁₄ alkyl. In some embodiments, R⁸ is optionally substituted C₂-C₁₄ alkenyl. In some embodiments, R⁸ is —(CH₂)ₘ-A-(CH₂)ₙH.

In some embodiments, R⁹ is optionally substituted C₁-C₁₄ alkyl, optionally substituted C₂-C₁₄ alkenyl, or —(CH₂)ₘ-A-(CH₂)ₙH. In some embodiments, R⁹ is optionally substituted C₃-C₁₀ alkyl. In some embodiments, R⁹ is optionally substituted C₄-C₁₀ alkyl. In some embodiments, R⁹ is optionally substituted C₅-C₁₀ alkyl. In some embodiments, R⁹ is optionally substituted C₉-C₁₀ alkyl. In some embodiments, R⁹ is optionally substituted C₁-C₁₄ alkyl. In some embodiments, R⁹ is optionally substituted C₂-C₁₄ alkenyl. In some embodiments, R⁹ is —(CH₂)ₘ-A-(CH₂)ₙH.

In some embodiments, each m is independently 0, 1, 2, 3, 4, 5, 6, 7, 8, 9, 10, 11, or 12. In some embodiments, each m is 0. In some embodiments, each m is 1. In some embodiments, each m is 2. In some embodiments, each m is 3. In some embodiments, each m is 4. In some embodiments, each m is 5. In some embodiments, each m is 6. In some embodiments, each m is 7. In some embodiments, each m is 8. In some embodiments, each m is 9. In some embodiments, each m is 10. In some embodiments, each m is 11. In some embodiments, each m is 12.

In some embodiments, each n is independently 0, 1, 2, 3, 4, 5, 6, 7, 8, 9, 10, 11, or 12. In some embodiments, each n is 0. In some embodiments, each n is 1. In some embodiments, each n is 2. In some embodiments, each n is 3. In some embodiments, each n is 4. In some embodiments, each n is 5. In some embodiments, each n is 6. In some embodiments, each n is 7. In some embodiments, each n is 8. In some embodiments, each n is 9. In some embodiments, each n is 10. In some embodiments, each n is 11. In some embodiments, each n is 12.

In some embodiments, each A is independently a C₃-C₈ cycloalkylenyl. In some embodiments, each A is cyclopropylenyl.

### X¹

In some embodiments, X¹ is optionally substituted C₂-C₆ alkylenyl. In some embodiments, X¹ is optionally substituted C₂-C₅ alkylenyl. In some embodiments, X¹ is optionally substituted C₂-C₄ alkylenyl. In some embodiments, X¹ is optionally substituted C₂-C₃ alkylenyl. In some embodiments, X¹ is optionally substituted C₂ alkylenyl. In some embodiments, X¹ is optionally substituted C₃ alkylenyl. In some embodiments, X¹ is optionally substituted C₄ alkylenyl. In some embodiments, X¹ is optionally substituted C₅ alkylenyl. In some embodiments, X¹ is optionally substituted C₆ alkylenyl. In some embodiments, X¹ is optionally substituted —(CH₂)₂—. In some embodiments, X¹ is optionally substituted —(CH₂)₃—. In some embodiments, X¹ is optionally substituted —(CH₂)₄—. In some embodiments, X¹ is optionally substituted —(CH₂)₅—. In some embodiments, X¹ is optionally substituted —(CH₂)₆—.

### X²

In some embodiments, X² is selected from the group consisting of a bond, —CH₂— and —CH₂CH₂—. In some embodiments, X² is a bond. In some embodiments, X² is —CH₂—. In some embodiments, X² is —CH₂CH₂—.

### X²′

In some embodiments, X²′ is selected from the group consisting of a bond, —CH₂— and —CH₂CH₂—. In some embodiments, X²′ is a bond. In some embodiments, X²′ is —CH₂—. In some embodiments, X²′ is —CH₂CH₂—.

### X³

In some embodiments, X³ is selected from the group consisting of a bond, —CH₂— and —CH₂CH₂—. In some embodiments, X³ is a bond. In some embodiments, X³ is —CH₂—. In some embodiments, X³ is —CH₂CH₂—.

### X³′

In some embodiments, X³ is selected from the group consisting of a bond, —CH₂— and —CH₂CH₂—. In some embodiments, X³′ is a bond. In some embodiments, X³′ is —CH₂—. In some embodiments, X³′ is —CH₂CH₂—.

### X⁴

In some embodiments, X⁴ is selected from the group consisting of optionally substituted C₂-C₁₄ alkylenyl and optionally substituted C₂-C₁₄ alkenylenyl. In some embodiments, X⁴ is optionally substituted C₂-C₁₄ alkylenyl. In some embodiments, X⁴ is optionally substituted C₂-C₁₀ alkylenyl. In some embodiments, X⁴ is optionally substituted C₂-C₈ alkylenyl. In some embodiments, X⁴ is optionally substituted C₂-C₆ alkylenyl. In some embodiments, X⁴ is optionally substituted C₃-C₆ alkylenyl. In some embodiments, X⁴ is optionally substituted C₃ alkylenyl. In some embodiments, X⁴ is optionally substituted C₄ alkylenyl. In some embodiments, X⁴ is optionally substituted C₅ alkylenyl. In some embodiments, X⁴ is optionally substituted C₆ alkylenyl. In some embodiments, X⁴ is optionally substituted —(CH₂)₂—. In some embodiments, X⁴ is optionally substituted —(CH₂)₃—. In some embodiments, X⁴ is optionally substituted —(CH₂)₄—. In some embodiments, X⁴ is optionally substituted —(CH₂)₅—. In some embodiments, X⁴ is optionally substituted —(CH₂)₆—.

### X⁵

In some embodiments, X⁵ is selected from the group consisting of optionally substituted C₂-C₁₄ alkylenyl and optionally substituted C₂-C₁₄ alkenylenyl. In some embodiments, X⁵ is optionally substituted C₂-C₁₄ alkylenyl. In some embodiments, X⁵ is optionally substituted C₂-C₁₀ alkylenyl. In some embodiments, X⁵ is optionally substituted C₂-C₈ alkylenyl. In some embodiments, X⁵ is optionally substituted C₂-C₆ alkylenyl. In some embodiments, X⁵ is optionally substituted C₃-C₆ alkylenyl. In some embodiments, X⁵ is optionally substituted C₃ alkylenyl. In some embodiments, X⁵ is optionally substituted C₄ alkylenyl. In some embodiments, X⁵ is optionally substituted C₅ alkylenyl. In some embodiments, X⁵ is optionally substituted C₆ alkylenyl. In some embodiments, X⁵ is optionally substituted —(CH₂)₂—. In some embodiments, X⁵ is optionally substituted —(CH₂)₃—. In some embodiments, X⁵ is optionally substituted —(CH₂)₄—. In some embodiments, X⁵ is optionally substituted —(CH₂)₅—. In some embodiments, X⁵ is optionally substituted —(CH₂)₆—.

### Y¹

In some embodiments, Y¹ is selected from the group consisting of

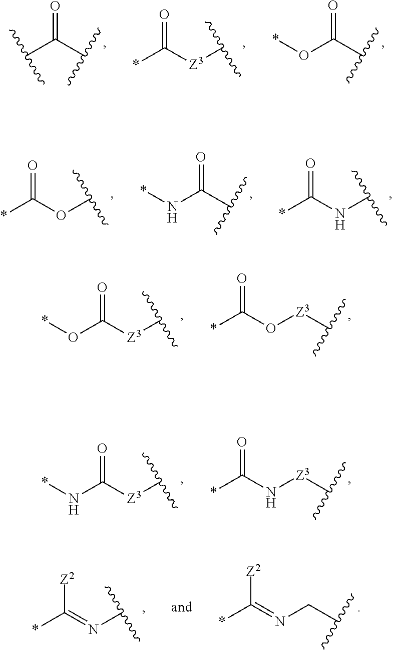

In some embodiments, Y¹ is

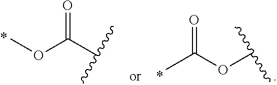

### Y²

In some embodiments, Y² is selected from the group consisting of

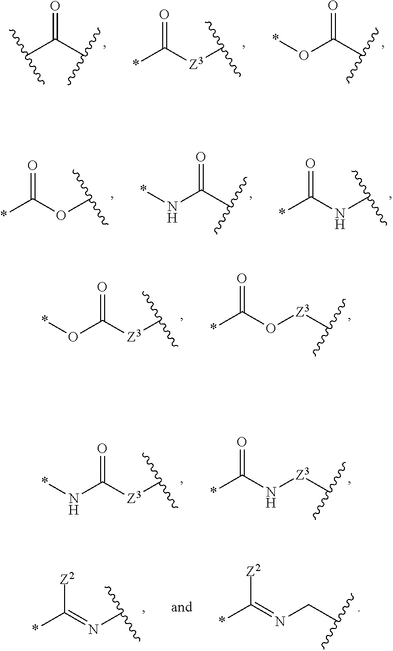

In some embodiments, Y² is

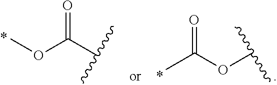

TABLE (II)
Non-Limiting Examples of Ionizable Lipids with a Cyclic Core

| | Compound |
|---|---|
| Structure | No. |
| 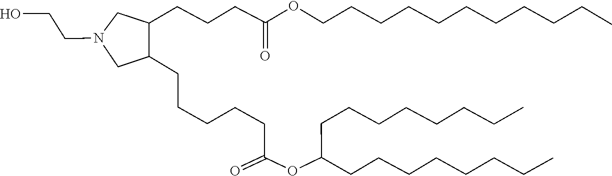 [SMILES: CCCCCCCCCCCOC(=O)CCCC1CN(CCO)CC1CCCCCC(=O)OC(CCCCCCCC)CCCCCCCC] | CY1 |
| 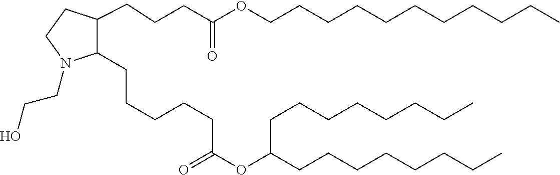 [SMILES: CCCCCCCCCCCOC(=O)CCCC1CCN(CCO)C1CCCCCC(=O)OC(CCCCCCCC)CCCCCCCC] | CY2 |
| 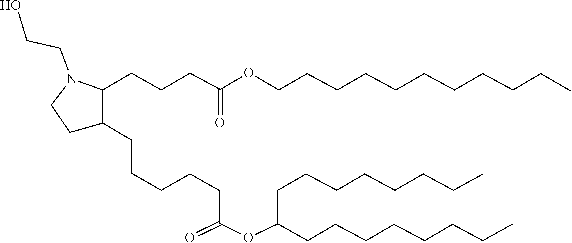 [SMILES: CCCCCCCCCCCOC(=O)CCCC1C(CCCCCC(=O)OC(CCCCCCCC)CCCCCCCC)CCN1CCO] | CY3 |
| 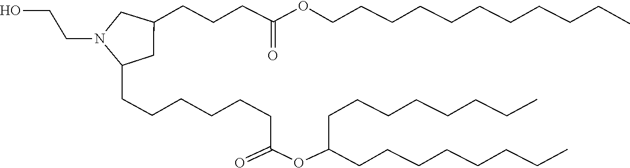 [SMILES: CCCCCCCCCCCOC(=O)CCCC1CC(CCCCCCC(=O)OC(CCCCCCCC)CCCCCCCC)N(CCO)C1] | CY4 |
| 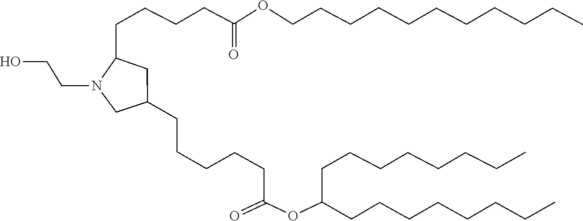 [SMILES: CCCCCCCCCCCOC(=O)CCCCC1CC(CCCCCC(=O)OC(CCCCCCCC)CCCCCCCC)CN1CCO] | CY5 |
| 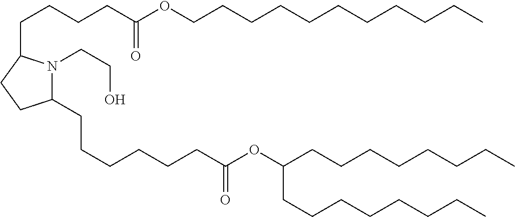 [SMILES: CCCCCCCCCCCOC(=O)CCCCC1CCC(CCCCCCC(=O)OC(CCCCCCCC)CCCCCCCC)N1CCO] | CY6 |
| 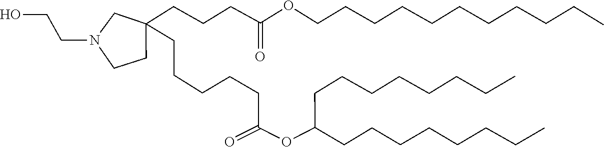 [SMILES: CCCCCCCCCCCOC(=O)CCCC1(CCCCCC(=O)OC(CCCCCCCC)CCCCCCCC)CCN(CCO)C1] | CY7 |
| 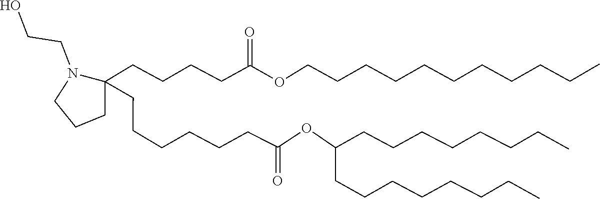 [SMILES: CCCCCCCCCCCOC(=O)CCCCC1(CCCCCCC(=O)OC(CCCCCCCC)CCCCCCCC)CCCN1CCO] | CY8 |
| 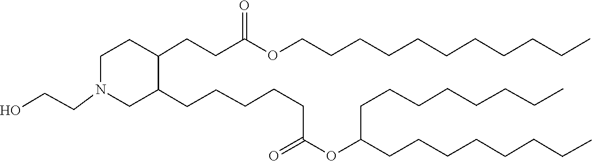 [SMILES: CCCCCCCCCCCOC(=O)CCC1CCN(CCO)CC1CCCCCC(=O)OC(CCCCCCCC)CCCCCCCC] | CY9 |
| 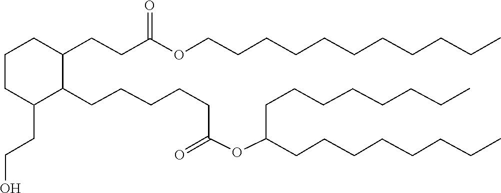 [SMILES: CCCCCCCCCCCOC(=O)CCC1CCCC(CCO)C1CCCCCC(=O)OC(CCCCCCCC)CCCCCCCC] | CY10 |
| 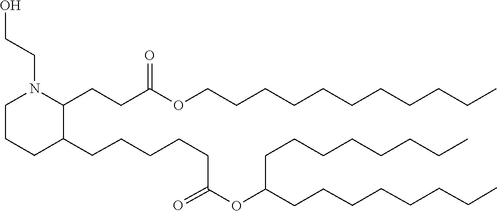 [SMILES: CCCCCCCCCCCOC(=O)CCC1C(CCCCCC(=O)OC(CCCCCCCC)CCCCCCCC)CCCN1CCO] | CY11 |
| 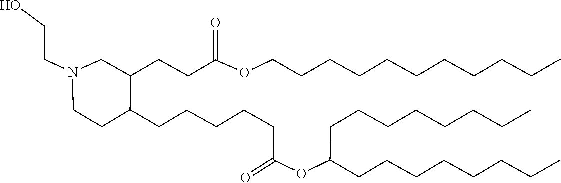 [SMILES: CCCCCCCCCCCOC(=O)CCC1CN(CCO)CCC1CCCCCC(=O)OC(CCCCCCCC)CCCCCCCC] | CY12 |
| 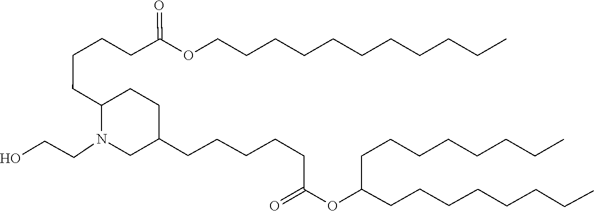 [SMILES: CCCCCCCCCCCOC(=O)CCCCC1CCC(CCCCCC(=O)OC(CCCCCCCC)CCCCCCCC)CN1CCO] | CY13 |
| 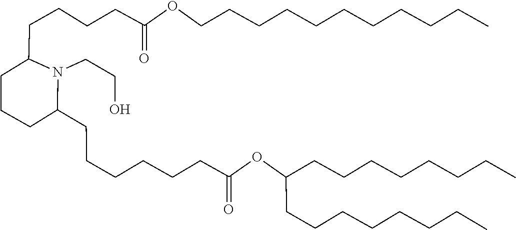 [SMILES: CCCCCCCCCCCOC(=O)CCCCC1CCCC(CCCCCCC(=O)OC(CCCCCCCC)CCCCCCCC)N1CCO] | CY14 |
| 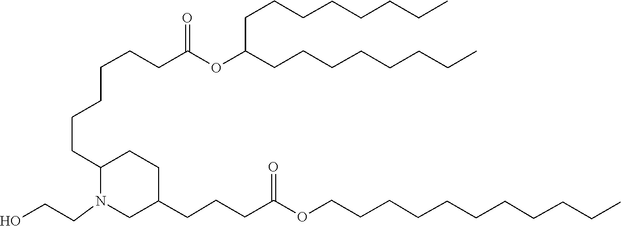 [SMILES: CCCCCCCCCCCOC(=O)CCCC1CCC(CCCCCCC(=O)OC(CCCCCCCC)CCCCCCCC)N(CCO)C1] | CY15 |
| 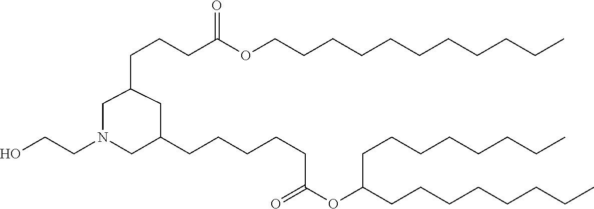 [SMILES: CCCCCCCCCCCOC(=O)CCCC1CC(CCCCCC(=O)OC(CCCCCCCC)CCCCCCCC)CN(CCO)C1] | CY16 |
| 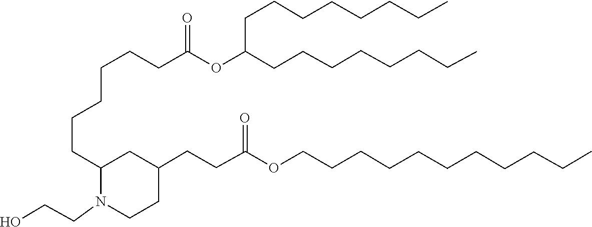 [SMILES: CCCCCCCCCCCOC(=O)CCC1CCN(CCO)C(CCCCCCC(=O)OC(CCCCCCCC)CCCCCCCC)C1] | CY17 |
| 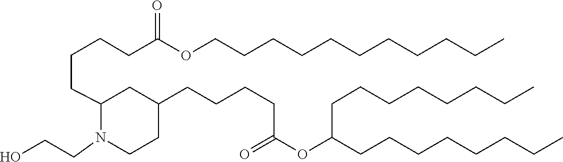 [SMILES: CCCCCCCCCCCOC(=O)CCCCC1CC(CCCCC(=O)OC(CCCCCCCC)CCCCCCCC)CCN1CCO] | CY18 |
| 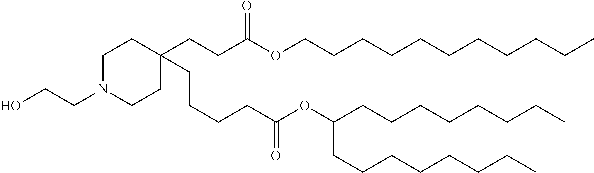 [SMILES: CCCCCCCCCCCOC(=O)CCC1(CCCCC(=O)OC(CCCCCCCC)CCCCCCCC)CCN(CCO)CC1] | CY19 |
| 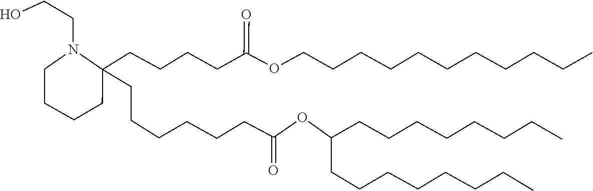 [SMILES: CCCCCCCCCCCOC(=O)CCCCC1(CCCCCCC(=O)OC(CCCCCCCC)CCCCCCCC)CCCCN1CCO] | CY20 |
| 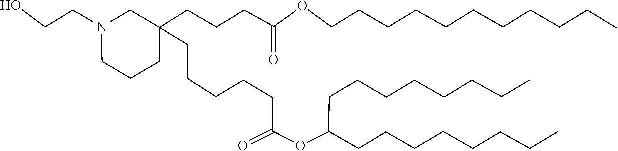 [SMILES: CCCCCCCCCCCOC(=O)CCCC1(CCCCCC(=O)OC(CCCCCCCC)CCCCCCCC)CCCN(CCO)C1] | CY21 |
| 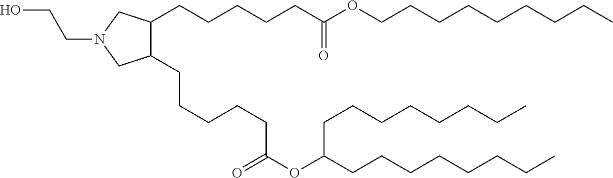 [SMILES: CCCCCCCCCOC(=O)CCCCCC1CN(CCO)CC1CCCCCC(=O)OC(CCCCCCCC)CCCCCCCC] | CY22 |
| 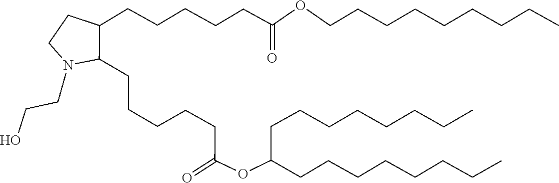 [SMILES: CCCCCCCCCOC(=O)CCCCCC1CCN(CCO)C1CCCCCC(=O)OC(CCCCCCCC)CCCCCCCC] | CY23 |
| 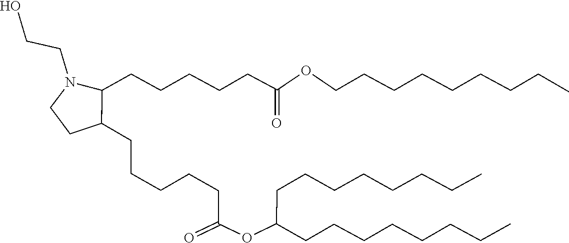 [SMILES: CCCCCCCCCOC(=O)CCCCCC1C(CCCCCC(=O)OC(CCCCCCCC)CCCCCCCC)CCN1CCO] | CY24 |
| 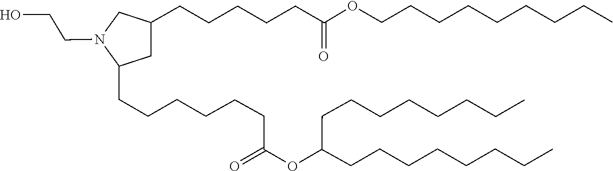 [SMILES: CCCCCCCCCOC(=O)CCCCCC1CC(CCCCCCC(=O)OC(CCCCCCCC)CCCCCCCC)N(CCO)C1] | CY25 |
| 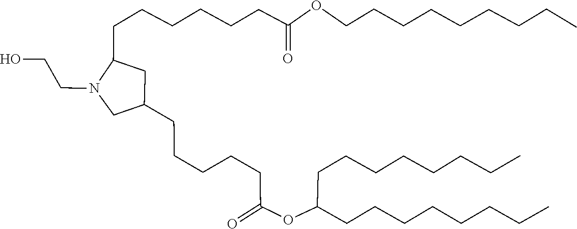 [SMILES: CCCCCCCCCOC(=O)CCCCCCC1CC(CCCCCC(=O)OC(CCCCCCCC)CCCCCCCC)CN1CCO] | CY26 |
| 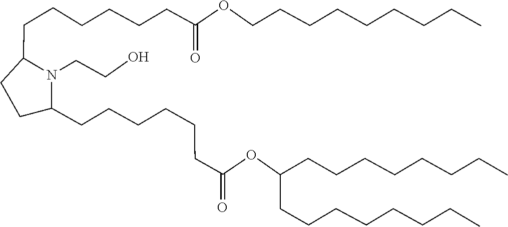 [SMILES: CCCCCCCCCOC(=O)CCCCCCC1CCC(CCCCCCC(=O)OC(CCCCCCCC)CCCCCCCC)N1CCO] | CY27 |
| 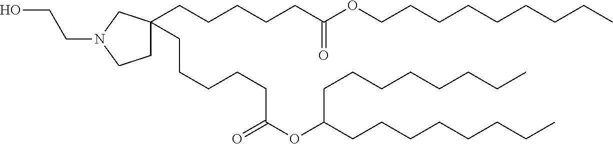 [SMILES: CCCCCCCCCOC(=O)CCCCCC1(CCCCCC(=O)OC(CCCCCCCC)CCCCCCCC)CCN(CCO)C1] | CY28 |
| 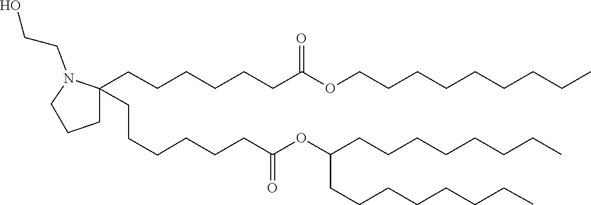 [SMILES: CCCCCCCCCOC(=O)CCCCCCC1(CCCCCCC(=O)OC(CCCCCCCC)CCCCCCCC)CCCN1CCO] | CY29 |
| 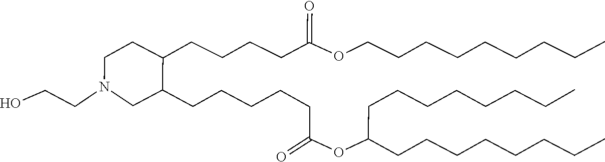 [SMILES: CCCCCCCCCOC(=O)CCCCC1CCN(CCO)CC1CCCCCC(=O)OC(CCCCCCCC)CCCCCCCC] | CY30 |
| 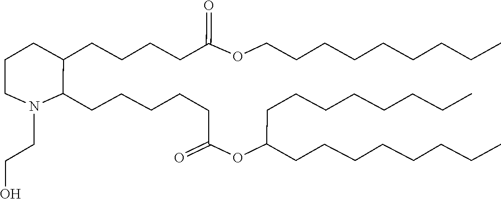 [SMILES: CCCCCCCCCOC(=O)CCCCC1CCCN(CCO)C1CCCCCC(=O)OC(CCCCCCCC)CCCCCCCC] | CY31 |
| 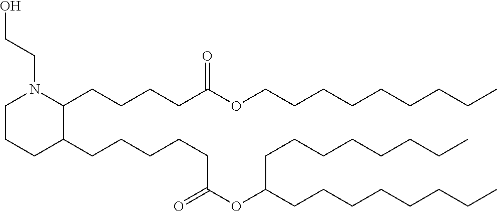 [SMILES: CCCCCCCCCOC(=O)CCCCC1C(CCCCCC(=O)OC(CCCCCCCC)CCCCCCCC)CCCN1CCO] | CY32 |
| 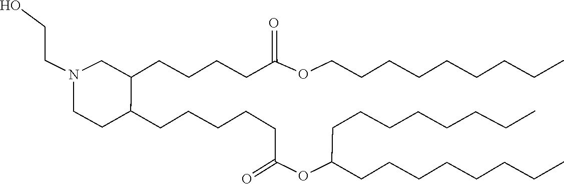 [SMILES: CCCCCCCCCOC(=O)CCCCC1CN(CCO)CCC1CCCCCC(=O)OC(CCCCCCCC)CCCCCCCC] | CY33 |
| 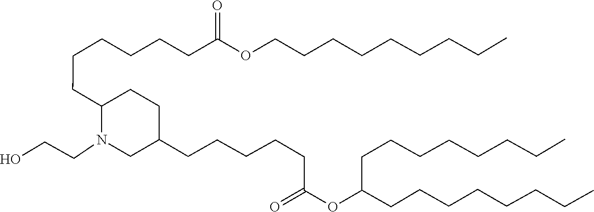 [SMILES: CCCCCCCCCOC(=O)CCCCCCC1CCC(CCCCCC(=O)OC(CCCCCCCC)CCCCCCCC)CN1CCO] | CY34 |
| 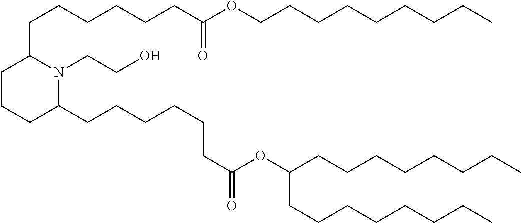 [SMILES: CCCCCCCCCOC(=O)CCCCCCC1CCCC(CCCCCCC(=O)OC(CCCCCCCC)CCCCCCCC)N1CCO] | CY35 |
| 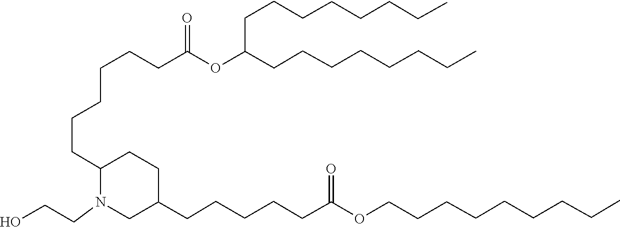 [SMILES: CCCCCCCCCOC(=O)CCCCCC1CCC(CCCCCCC(=O)OC(CCCCCCCC)CCCCCCCC)N(CCO)C1] | CY36 |
| 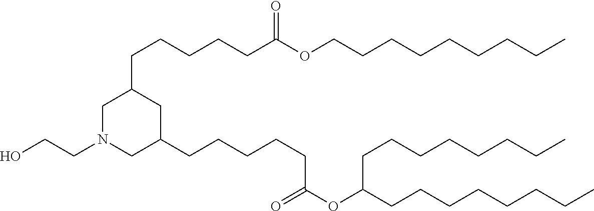 [SMILES: CCCCCCCCCOC(=O)CCCCCC1CC(CCCCCC(=O)OC(CCCCCCCC)CCCCCCCC)CN(CCO)C1] | CY37 |
| 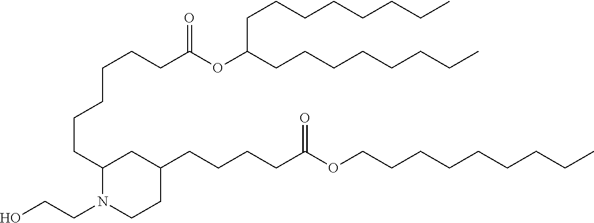 [SMILES: CCCCCCCCCOC(=O)CCCCC1CCN(CCO)C(CCCCCCC(=O)OC(CCCCCCCC)CCCCCCCC)C1] | CY38 |
| 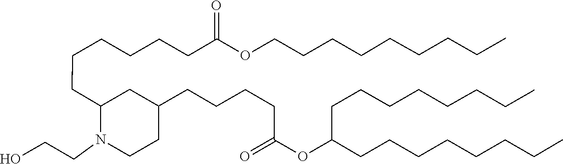 [SMILES: CCCCCCCCCOC(=O)CCCCCCC1CC(CCCCC(=O)OC(CCCCCCCC)CCCCCCCC)CCN1CCO] | CY39 |
| 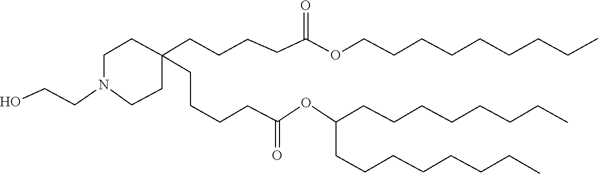 [SMILES: CCCCCCCCCOC(=O)CCCCC1(CCCCC(=O)OC(CCCCCCCC)CCCCCCCC)CCN(CCO)CC1] | CY40 |
| 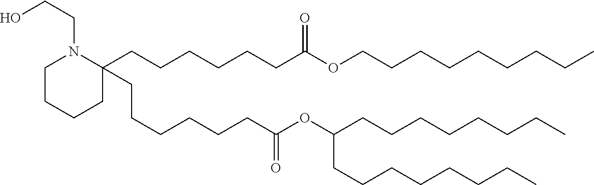 [SMILES: CCCCCCCCCOC(=O)CCCCCCC1(CCCCCCC(=O)OC(CCCCCCCC)CCCCCCCC)CCCCN1CCO] | CY41 |
| 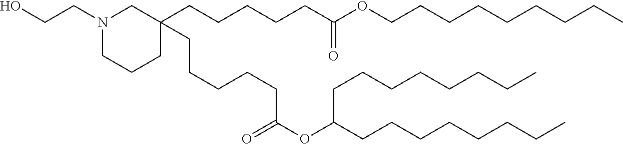 [SMILES: CCCCCCCCCOC(=O)CCCCCC1(CCCCCC(=O)OC(CCCCCCCC)CCCCCCCC)CCCN(CCO)C1] | CY42 |
| 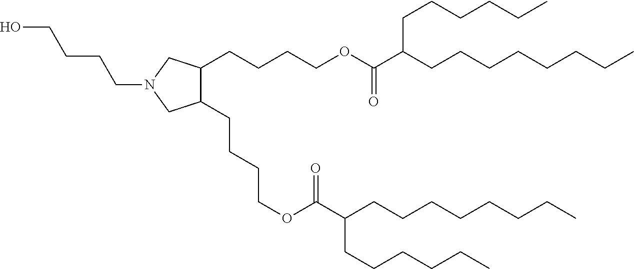 [SMILES: CCCCCCCCC(CCCCCC)C(=O)OCCCCC1CN(CCCCO)CC1CCCCOC(=O)C(CCCCCC)CCCCCCCC] | CY43 |
| 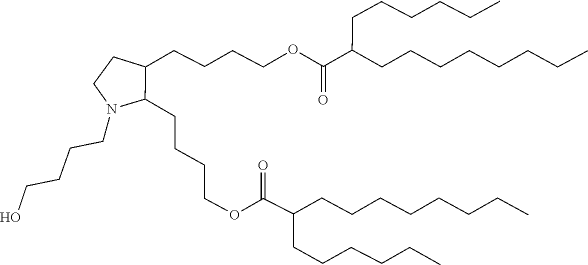 [SMILES: CCCCCCCCC(CCCCCC)C(=O)OCCCCC1CCN(CCCCO)C1CCCCOC(=O)C(CCCCCC)CCCCCCCC] | CY44 |
| 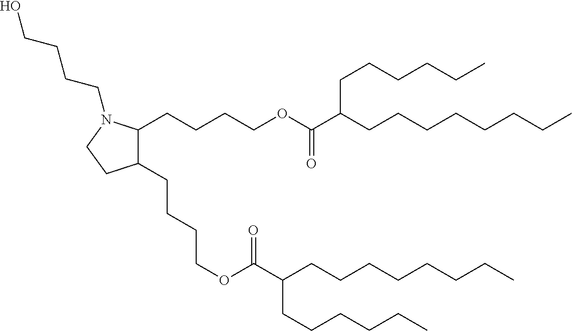 [SMILES: CCCCCCCCC(CCCCCC)C(=O)OCCCCC1CCN(CCCCO)C1CCCCOC(=O)C(CCCCCC)CCCCCCCC] | CY45 |
| 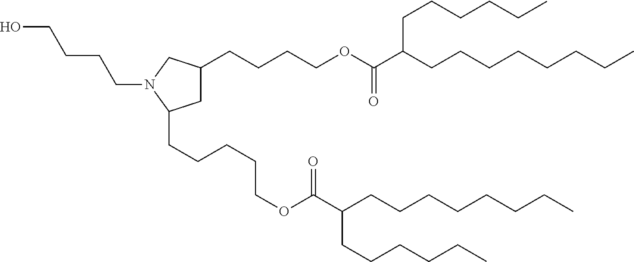 [SMILES: CCCCCCCCC(CCCCCC)C(=O)OCCCCCC1CC(CCCCOC(=O)C(CCCCCC)CCCCCCCC)CN1CCCCO] | CY46 |
| 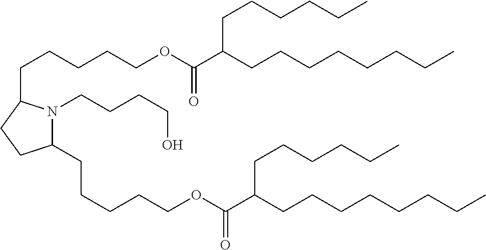 [SMILES: CCCCCCCCC(CCCCCC)C(=O)OCCCCCC1CCC(CCCCCOC(=O)C(CCCCCC)CCCCCCCC)N1CCCCO] | CY47 |
| 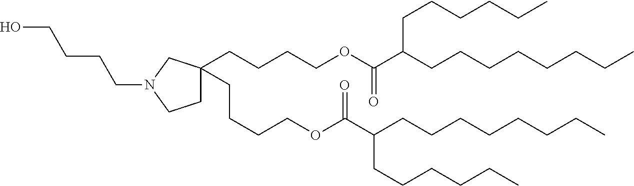 [SMILES: CCCCCCCCC(CCCCCC)C(=O)OCCCCC1(CCCCOC(=O)C(CCCCCC)CCCCCCCC)CCN(CCCCO)C1] | CY48 |
| 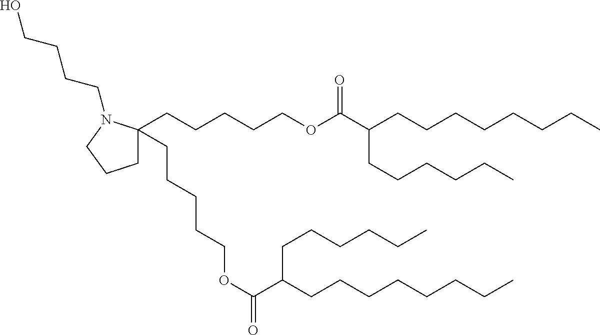 [SMILES: CCCCCCCCC(CCCCCC)C(=O)OCCCCCC1(CCCCCOC(=O)C(CCCCCC)CCCCCCCC)CCCN1CCCCO] | CY49 |
| 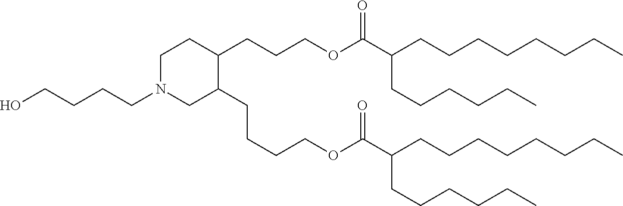 [SMILES: CCCCCCCCC(CCCCCC)C(=O)OCCCCC1CN(CCCCO)CCC1CCCOC(=O)C(CCCCCC)CCCCCCCC] | CY50 |
| 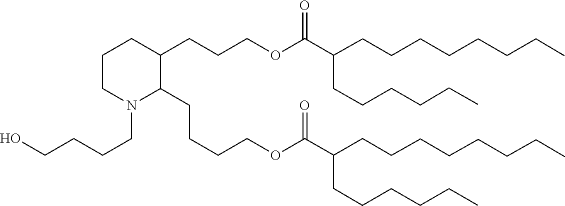 [SMILES: CCCCCCCCC(CCCCCC)C(=O)OCCCCC1C(CCCOC(=O)C(CCCCCC)CCCCCCCC)CCCN1CCCCO] | CY51 |
| 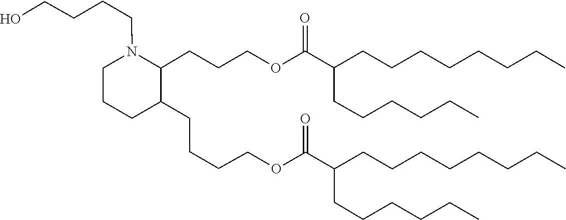 [SMILES: CCCCCCCCC(CCCCCC)C(=O)OCCCCC1CCCN(CCCCO)C1CCCOC(=O)C(CCCCCC)CCCCCCCC] | CY52 |
| 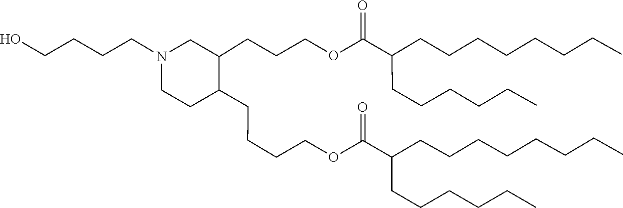 [SMILES: CCCCCCCCC(CCCCCC)C(=O)OCCCCC1CCN(CCCCO)CC1CCCOC(=O)C(CCCCCC)CCCCCCCC] | CY53 |
| 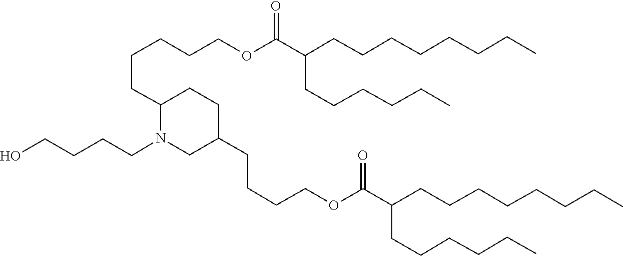 [SMILES: CCCCCCCCC(CCCCCC)C(=O)OCCCCCC1CCC(CCCCOC(=O)C(CCCCCC)CCCCCCCC)CN1CCCCO] | CY54 |
| 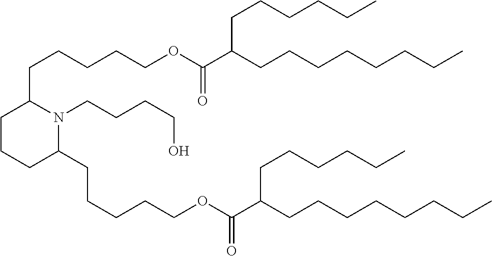 [SMILES: CCCCCCCCC(CCCCCC)C(=O)OCCCCCC1CCCC(CCCCCOC(=O)C(CCCCCC)CCCCCCCC)N1CCCCO] | CY55 |
| 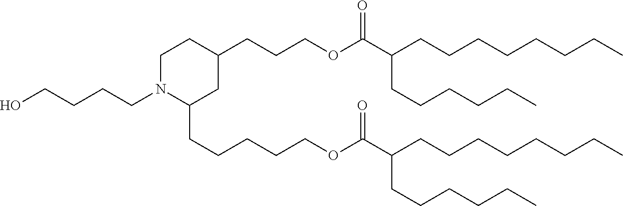 [SMILES: CCCCCCCCC(CCCCCC)C(=O)OCCCCCC1CC(CCCOC(=O)C(CCCCCC)CCCCCCCC)CCN1CCCCO] | CY56 |
| 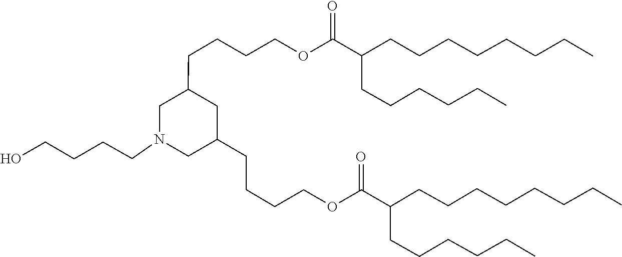 [SMILES: CCCCCCCCC(CCCCCC)C(=O)OCCCCC1CC(CCCCOC(=O)C(CCCCCC)CCCCCCCC)CN(CCCCO)C1] | CY57 |
| 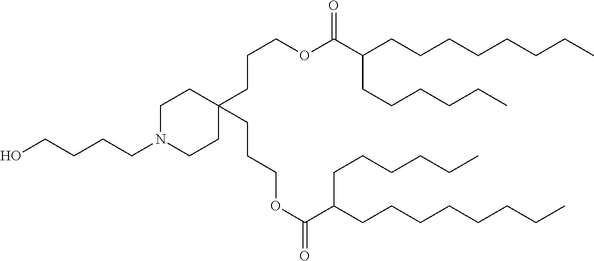 [SMILES: CCCCCCCCC(CCCCCC)C(=O)OCCCC1(CCCOC(=O)C(CCCCCC)CCCCCCCC)CCN(CCCCO)CC1] | CY58 |
| 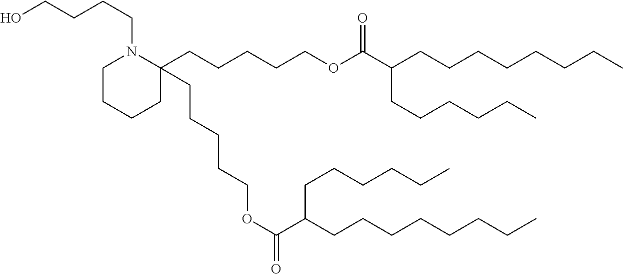 [SMILES: CCCCCCCCC(CCCCCC)C(=O)OCCCCCC1(CCCCCOC(=O)C(CCCCCC)CCCCCCCC)CCCCN1CCCCO] | CY59 |
| 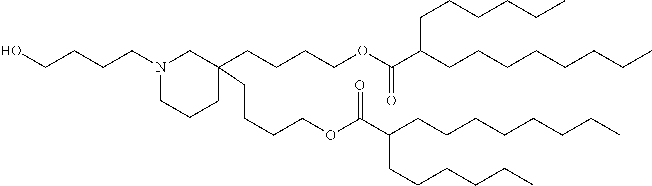 [SMILES: CCCCCCCCC(CCCCCC)C(=O)OCCCCC1(CCCCOC(=O)C(CCCCCC)CCCCCCCC)CCCN(CCCCO)C1] | CY60 |
| 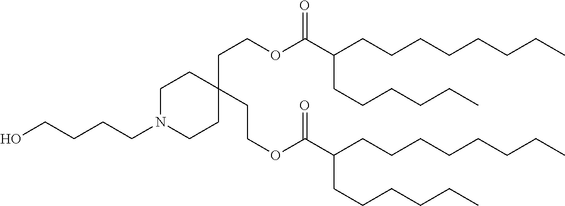 [SMILES: CCCCCCCCC(CCCCCC)C(=O)OCCC1(CCOC(=O)C(CCCCCC)CCCCCCCC)CCN(CCCCO)CC1] | CY61 |
| 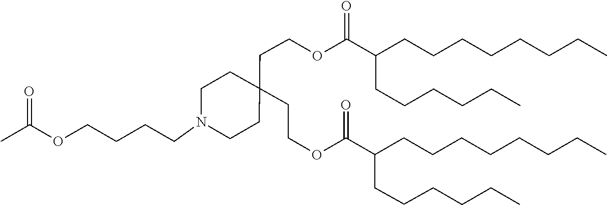 [SMILES: CCCCCCCCC(CCCCCC)C(=O)OCCC1(CCOC(=O)C(CCCCCC)CCCCCCCC)CCN(CCCCOC(C)=O)CC1] | CY62 |
| 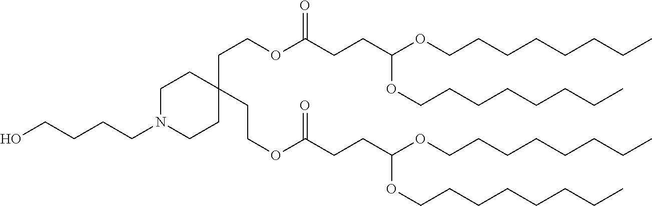 [SMILES: CCCCCCCCOC(CCC(=O)OCCC1(CCOC(=O)CCC(OCCCCCCCC)OCCCCCCCC)CCN(CCCCO)CC1)OCCCCCCCC] | CY63 |
| 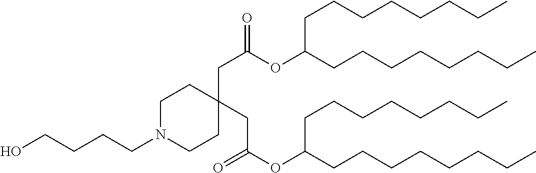 [SMILES: CCCCCCCCC(CCCCCCCC)OC(=O)CC1(CC(=O)OC(CCCCCCCC)CCCCCCCC)CCN(CCCCO)CC1] | CY64 |
| 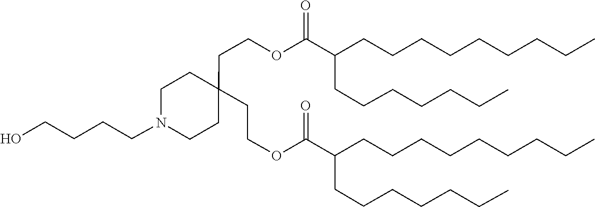 [SMILES: CCCCCCCCCC(CCCCCCC)C(=O)OCCC1(CCOC(=O)C(CCCCCCC)CCCCCCCCC)CCN(CCCCO)CC1] | CY65 |
| 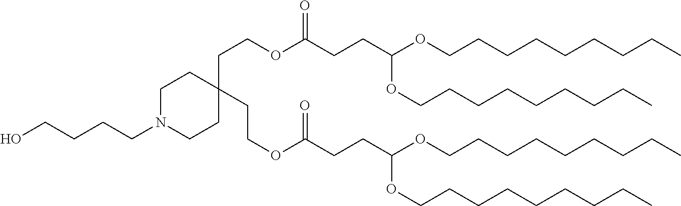 [SMILES: CCCCCCCCCOC(CCC(=O)OCCC1(CCOC(=O)CCC(OCCCCCCCCC)OCCCCCCCCC)CCN(CCCCO)CC1)OCCCCCCCCC] | CY66 |
| 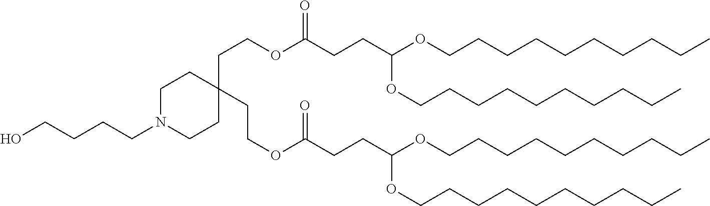 [SMILES: CCCCCCCCCCOC(CCC(=O)OCCC1(CCOC(=O)CCC(OCCCCCCCCCC)OCCCCCCCCCC)CCN(CCCCO)CC1)OCCCCCCCCCC] | CY67 |
| 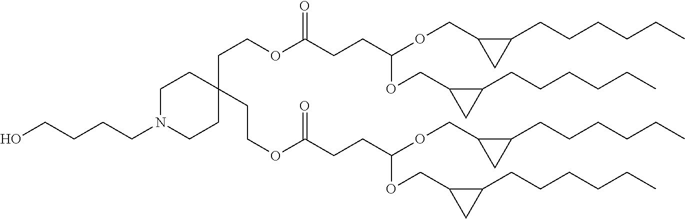 [SMILES: CCCCCCC1CC1COC(CCC(=O)OCCC1(CCOC(=O)CCC(OCC2CC2CCCCCC)OCC2CC2CCCCCC)CCN(CCCCO)CC1)OCC1CC1CCCCCC] | CY68 |
| 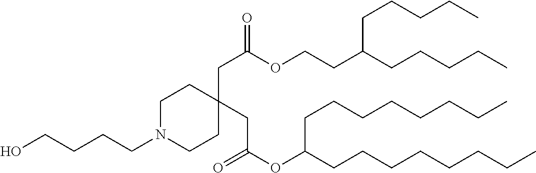 [SMILES: CCCCCCCCC(CCCCCCCC)OC(=O)CC1(CC(=O)OCCC(CCCCC)CCCCC)CCN(CCCCO)CC1] | CY69 |
| 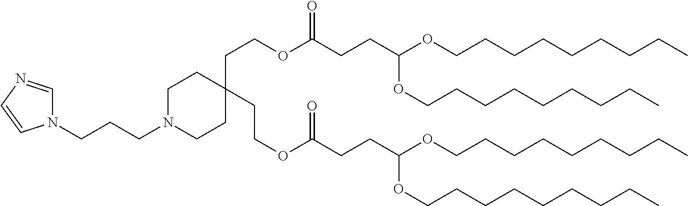 [SMILES: CCCCCCCCCOC(CCC(=O)OCCC1(CCOC(=O)CCC(OCCCCCCCCC)OCCCCCCCCC)CCN(CCCn2ccnc2)CC1)OCCCCCCCCC] | CY70 |
| 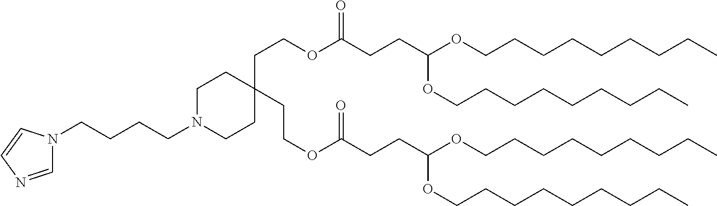 [SMILES: CCCCCCCCCOC(CCC(=O)OCCC1(CCOC(=O)CCC(OCCCCCCCCC)OCCCCCCCCC)CCN(CCCCn2ccnc2)CC1)OCCCCCCCCC] | CY71 |

In some embodiments, an LNP of the present disclosure comprises an ionizable lipid disclosed in PCT Application PCT/US2022/082276, which is incorporated by reference herein, in its entirety.

In one embodiment, the disclosure provides a compound of Formula IA:

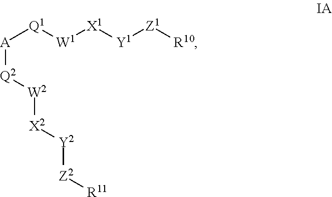

or a pharmaceutically acceptable salt or solvate thereof, wherein:
A is selected from the group consisting of —N(R^{1a})— and —C(R¹)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

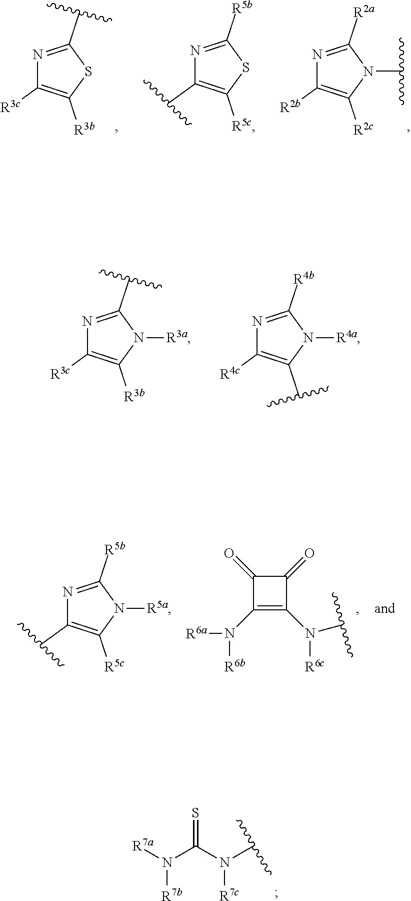

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and Rec is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;

R⁸ is selected from the group consisting of —NR^{9a}R^{9b}

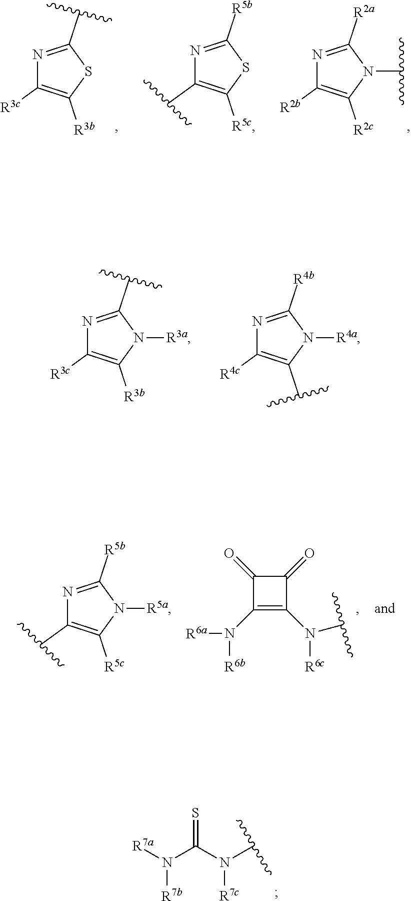

R^{3a} and R^{9b} are independently selected from the group consisting of hydrogen and C₁-C₆ alkyl; or
R^{9a} and Rob taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₄-C₁₂ cycloalkylenyl,

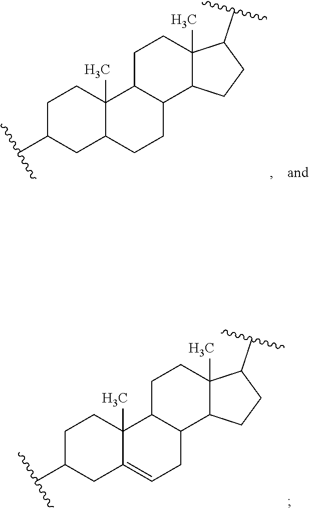 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R¹²)—, and —OC(═O)O—;
R^{12b} is selected from the group consisting of hydrogen and C₁-C₆ alkyl;
X² is optionally substituted C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, optionally substituted C₄-C₁₂ cycloalkylenyl,

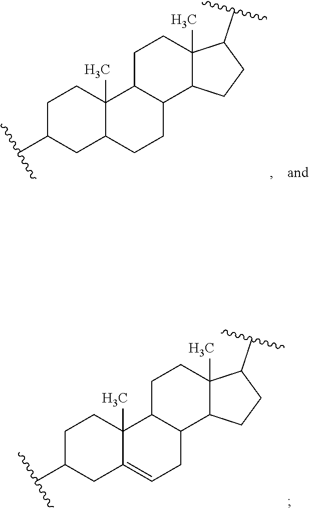 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In one embodiment, the disclosure provides a compound of Formula IB:

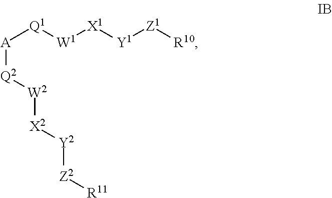

or a pharmaceutically acceptable salt or solvate thereof, wherein:
A is selected from the group consisting of —N(R^{1a})— and —C(R′)—OC(═O)(R^{8a})—;
R^{1a} is -L¹-R¹,
L¹ is C₂-C₆ alkylenyl or —(CH₂)₂₋₆—OC(═O)—;
R¹ is selected from the group consisting of —OH,

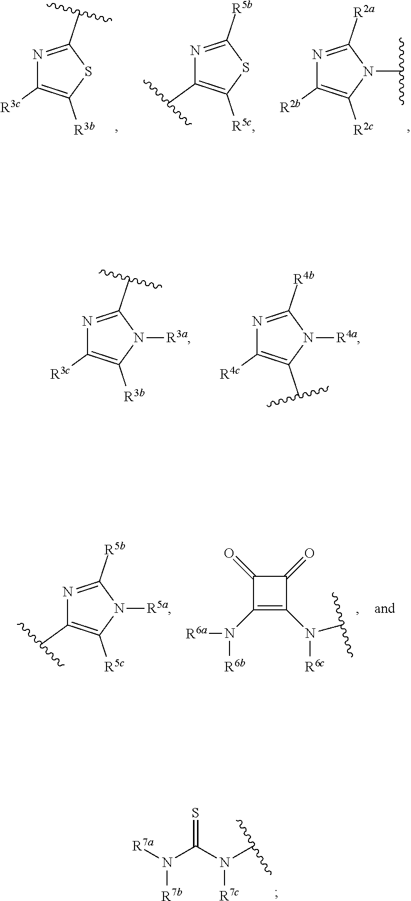

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl; R^{8a} is -L²-R³;
L² is C₂-C₆ alkylenyl;
R⁸ is selected from the group consisting of —NR^{9a}R^{9b},

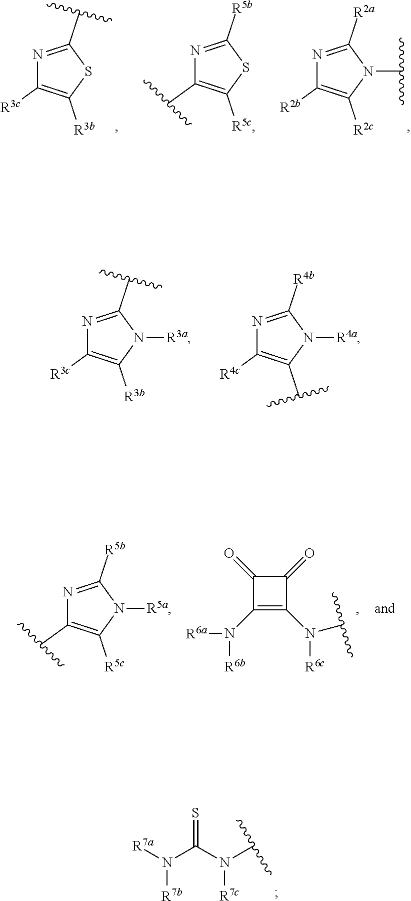

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, — N(R^{12a})C(═O)O—, and —OC(═O)O—;
R^{12a} is selected from the group consisting of hydrogen and C₁-C₆ alkyl;
X¹ is optionally substituted C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₅-C₁₂ bridged cycloalkylenyl,

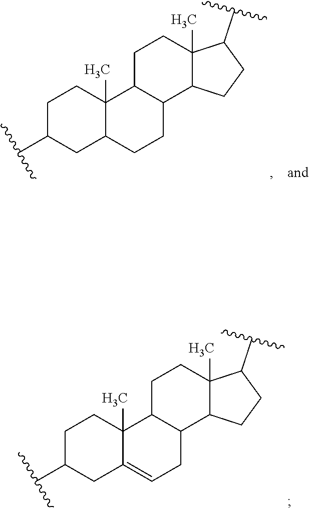 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})— and —OC(═O)O—;
R^{12b} is selected from the group consisting of hydrogen and C₁-C₆ alkyl;
X² is optionally substituted C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, optionally substituted C₄-C₁₂ cycloalkylenyl,

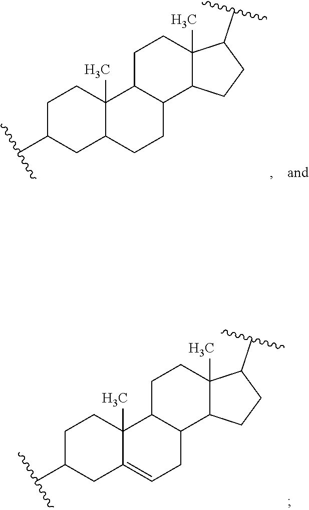 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹l is selected from the group consisting of hydrogen, C₁-C₁₀ alkyl, and C₂-C₁₀ alkenyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In one embodiment, the disclosure provides a compound of Formula IC:

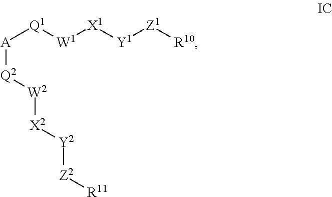

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

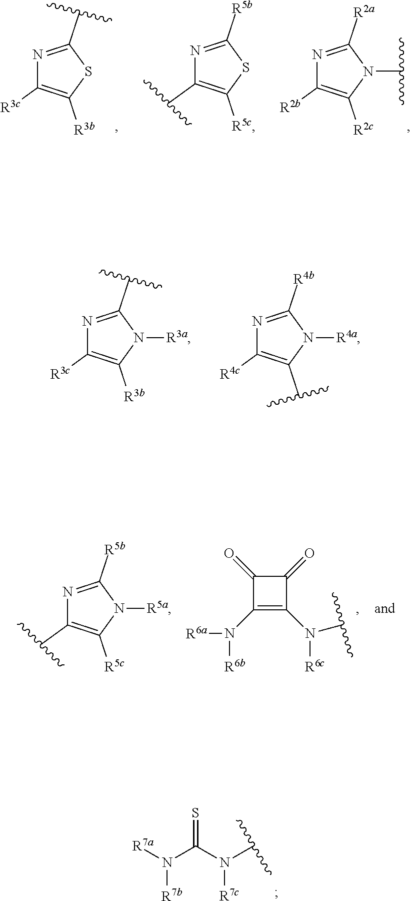

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl; R^{8a} is -L²-R³;
L² is C₂-C₆ alkylenyl;
R⁸ is selected from the group consisting of —NR^{9a}R^{9b}

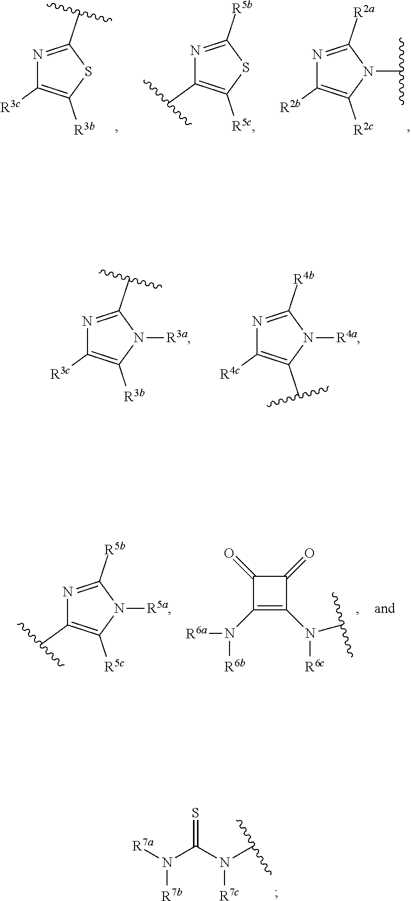

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted branched C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₄-C₁₂ cycloalkylenyl,

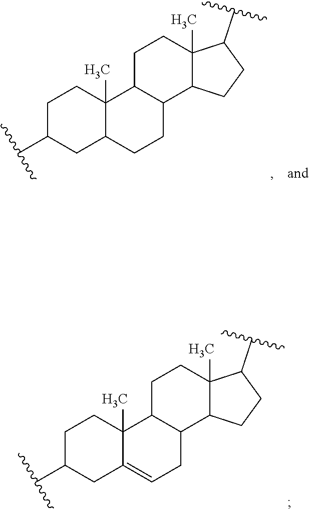 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
Y² is selected from the group consisting of —(CH₂)ₙ—, —O—, —S—, and —S—S—;
n is 0, 1, 2, 3, 4, 5, or 6;
Z² is of —(CH₂)ₚ—;
p is 0 or 1; and
R¹¹ is C₁-C₂₀ branched alkyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In some embodiments, the disclosure provides a compound of any one of Formulae IA, IB, IC, or a pharmaceutically acceptable salt or solvate thereof, wherein Z¹ is optionally substituted C₅-C₁₂ bridged cycloalkylenyl.

In some embodiments, the disclosure provides a compound of any one of Formulae IA, IB, IC, or a pharmaceutically acceptable salt or solvate thereof, wherein Z¹ is not adamantyl.

In one embodiment, the disclosure provides a compound of Formula ID:

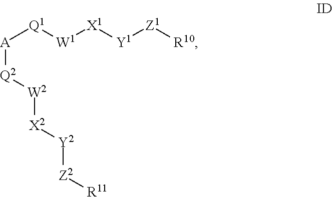

or a pharmaceutically acceptable salt or solvate thereof, wherein:
A is selected from the group consisting of —N(R^{1a})— and —C(R′)—OC(═O)(R^{8a})—; R^{1a} is -L¹-R¹;
L′ is C₂-C₆ alkylenyl or —(CH₂)₂₋₆—OC(═O)—;
R¹ is selected from the group consisting of —OH,

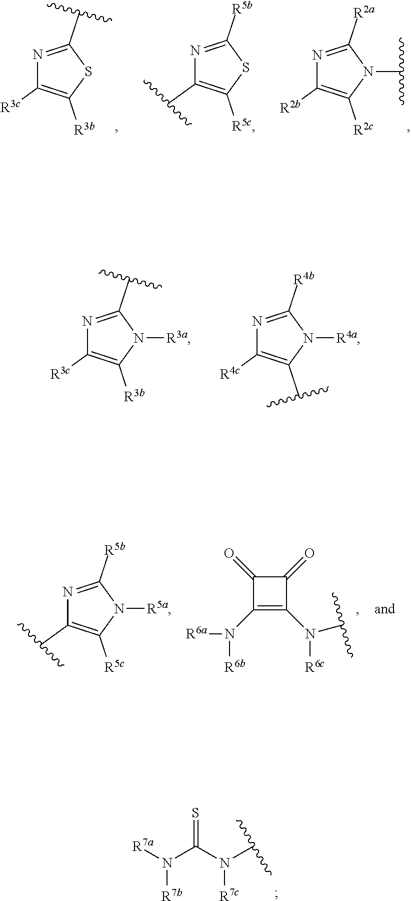

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸,
L² is C₂-C₆ alkylenyl;
R⁸ is selected from the group consisting of —NR^{9a}R^{9b},

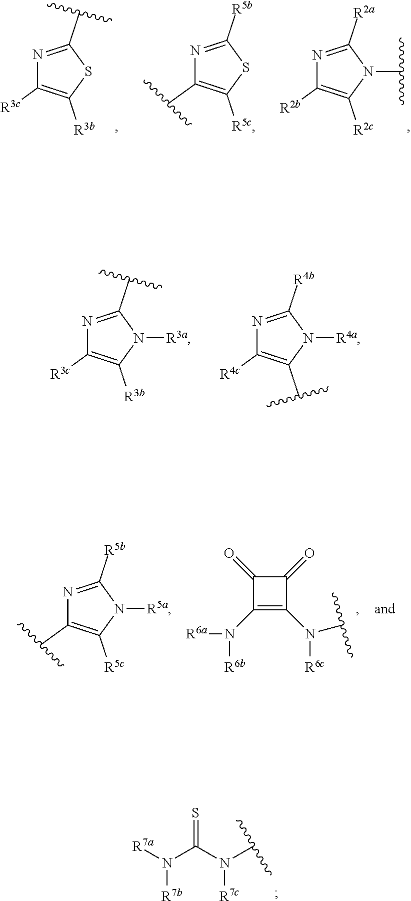

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted branched C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is optionally substituted C₅-C₁₂ bridged cycloalkylenyl;
R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
Y² is —(CH₂)ₙ—;
n is 0, 1, 2, 3, 4, 5, or 6;
Z² is of —(CH₂)ₚ—;
p is 0 or 1; and
R¹¹ is C₁-C₂₀ branched alkyl.

In some embodiments, the disclosure provides a compound of Formula ID or a pharmaceutically acceptable salt or solvate thereof, wherein Z¹ is not adamantyl.

In one embodiment, the disclosure provides a compound of Formula I:

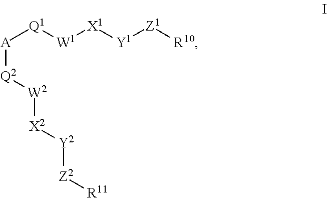

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl;
R¹ is selected from the group consisting of —OH,

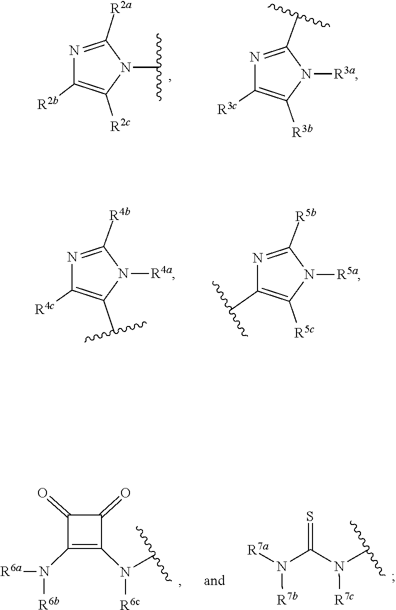

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R³;
L² is C₂-C₆ alkylenyl;
R⁸ is —NR^{9a}R^{9b};
R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of C₄-C₁₂ cycloalkylenyl,

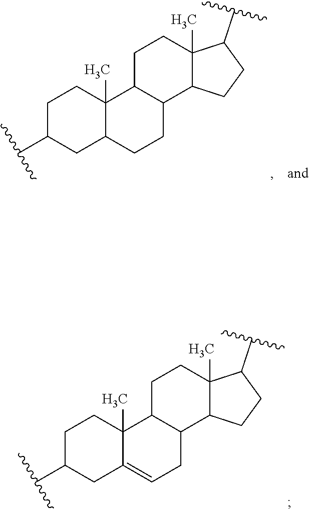 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, C₄-C₁₂ cycloalkylenyl,

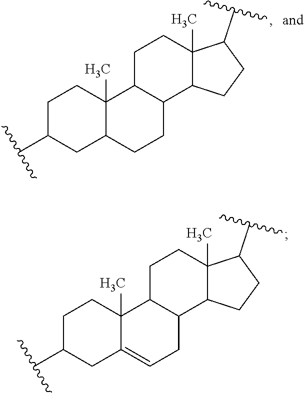 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl.

In another embodiment, the disclosure provides a compound of Formula II:

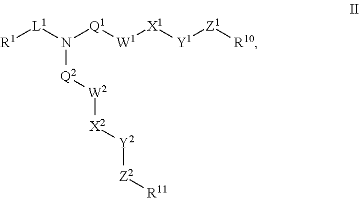

or a pharmaceutically acceptable salt or solvate thereof, wherein R¹, R¹⁰, R¹¹, Q¹, Q², W¹, W² X¹, X², Y¹, Y², Z¹, and Z² are as defined herein in Formula IA, Formula IB, Formula IC, Formula ID, Formula I, or below.

In another embodiment, the disclosure provides a compound of Formula III:

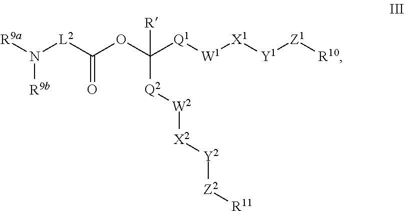

or a pharmaceutically acceptable salt or solvate thereof, wherein R′, R^{9a}, R⁹%, R¹⁰, R¹¹, L², Q¹, Q′, W¹, W², X¹, X², Y¹, Y², Z¹, and Z² are as defined herein in Formula IA, Formula IB, Formula IC, Formula ID, Formula I, or below.

In another embodiment, the disclosure provides a compound of Formula IV:

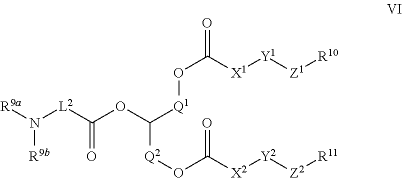

or a pharmaceutically acceptable salt or solvate thereof, wherein R⁹, R⁹, L², Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VI′:

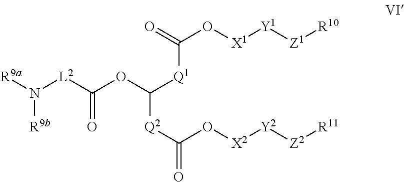

or a pharmaceutically acceptable salt or solvate thereof, wherein R^{9a}, R^{9b}, L², Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VI″:

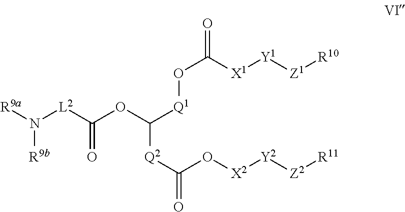

or a pharmaceutically acceptable salt or solvate thereof, wherein R^{9a}, R^{9b}, L², Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VI″″:

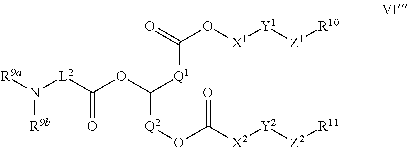

or a pharmaceutically acceptable salt or solvate thereof, wherein R^{9a}, R^{9b}, L², Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VII:

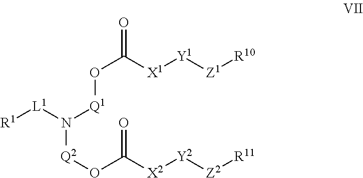

or a pharmaceutically acceptable salt or solvate thereof, wherein R¹, L¹, Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VII′:

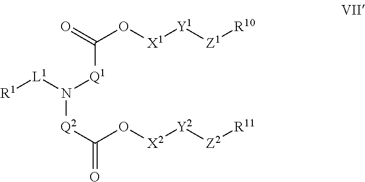

or a pharmaceutically acceptable salt or solvate thereof, wherein R¹, L¹, Q¹, Q², X¹, X², Y¹, Y², Z¹, Z², R¹⁰, an R¹¹ are as defined herein in Formula IA, Formula IB, Formula IC, Formula ID, Formula I, or below,

In another embodiment, the disclosure provides a compound of Formula VII″:

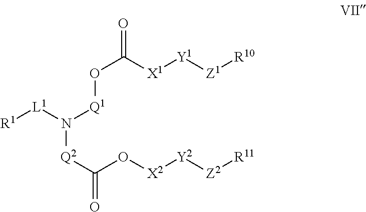

or a pharmaceutically acceptable salt or solvate thereof, wherein R¹, L¹, Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VII″′:

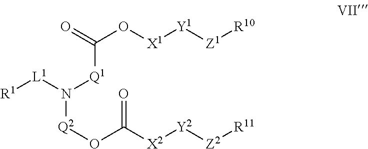

or a pharmaceutically acceptable salt or solvate thereof, wherein R¹, L¹, Q¹, Q², X¹, X², Y¹, Y², Z¹, Z², R¹⁰, an R¹¹ are as defined herein in Formula IA, Formula IB, Formula IC, Formula ID, Formula I, or below.

Formula IA, Formula IB, Formula IC, Formula I, In another embodiment, the disclosure provides a compound of Formula VIII:

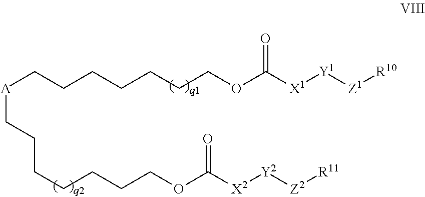

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII, wherein Z¹ is an optionally substituted C₅-C₁₂ bridged cycloalkylenyl. In another embodiment, the disclosure provides a compound of Formula VIII′:

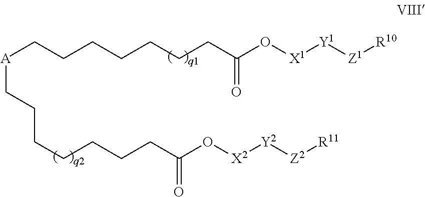

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII′, wherein Z¹ is an optionally substituted C₅-C₁₂ bridged cycloalkylenyl.

In another embodiment, the disclosure provides a compound of Formula VIII″:

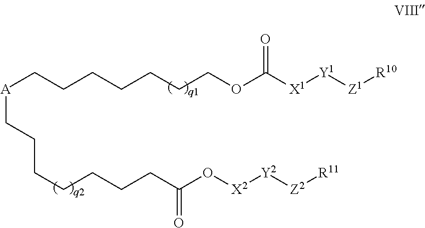

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII″, wherein Z¹ is an optionally substituted C₅-C₁₂ bridged cycloalkylenyl.

In another embodiment, the disclosure provides a compound of Formula VIII′″:

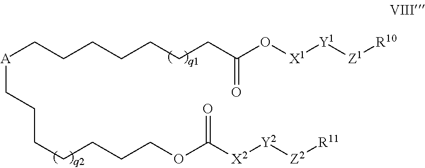

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII″, wherein Z¹ is an optionally substituted C₅-C₁₂ bridged cycloalkylenyl.

In another embodiment, the disclosure provides a compound of Formula IX:

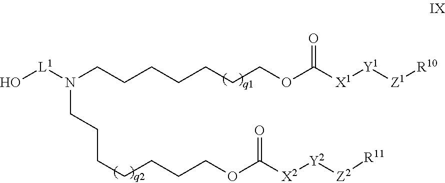

or a pharmaceutically acceptable salt or solvate thereof, wherein
q¹ is 0, 1, 2, or 3;
q² is 0, 1, 2, or 3;
L¹, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula IX, wherein Z¹ is an optionally substituted C₅-C₁₂ bridged cycloalkylenyl.

In another embodiment, the disclosure provides a compound of Formula IX′:

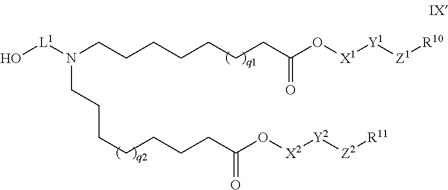

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula IX′, wherein Z¹ is an optionally substituted C₅-C₁₂ bridged cycloalkylenyl.

In another embodiment, the disclosure provides a compound of Formula IX″:

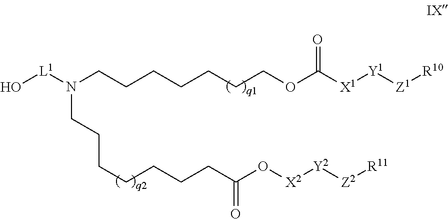

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula IX″, wherein Z¹ is an optionally substituted C₅-C₁₂ bridged cycloalkylenyl.

In another embodiment, the disclosure provides a compound of Formula IX″:

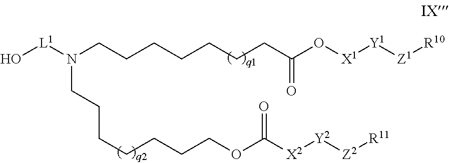

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula IX″′, wherein Z¹ is an optionally substituted C₅-C₁₂ bridged cycloalkylenyl.

In another embodiment, the disclosure provides a compound of Formula X:

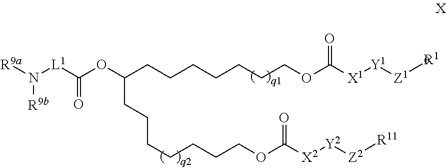

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R^{9a}, R⁹%, R¹⁰, an R¹¹ are as defined herein in Formula IA, Formula IB, Formula IC, Formula ID, Formula I or below.

In certain embodiments, the compound is a compound of Formula X, wherein Z¹ is an optionally substituted C₅-C₁₂ bridged cycloalkylenyl.

In another embodiment, the disclosure provides a compound of Formula X′:

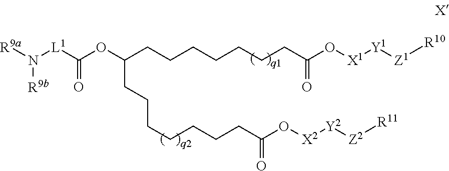

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R^{9a}, R⁹%, R¹⁰, an R¹¹ are as defined herein in Formula IA, Formula IB, Formula IC, Formula ID, Formula I or below.

In certain embodiments, the compound is a compound of Formula X′, wherein Z¹ is an optionally substituted C₅-C₁₂ bridged cycloalkylenyl.

In another embodiment, the disclosure provides a compound of Formula X′

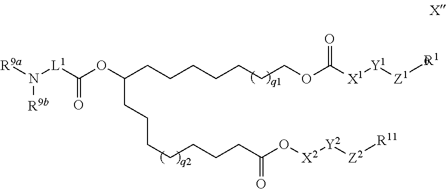

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R^{9a}, R^{9b}, R¹⁰, an R¹¹ are as defined herein in Formula IA, Formula IB, Formula IC, Formula ID, Formula I or below.

In certain embodiments, the compound is a compound of Formula X″, wherein Z¹ is an optionally substituted C₅-C₁₂ bridged cycloalkylenyl.

In another embodiment, the disclosure provides a compound of Formula X″′:

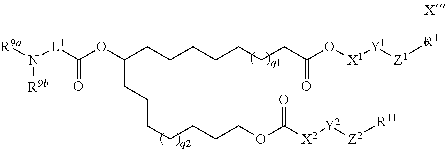

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R^{9a}, R^{9b}, R¹⁰, an R¹¹ are as defined herein in Formula IA, Formula IB, Formula IC, Formula ID, Formula I or below.

In certain embodiments, the compound is a compound of Formula X″″, wherein Z¹ is an optionally substituted C₅-C₁₂ bridged cycloalkylenyl.

In another embodiment, the disclosure provides a compound of Formula XI:

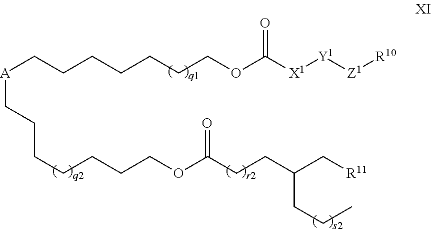

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XI, wherein Z¹ is an optionally substituted C₅-C₁₂ bridged cycloalkylenyl.

In another embodiment, the disclosure provides a compound of Formula XI′:

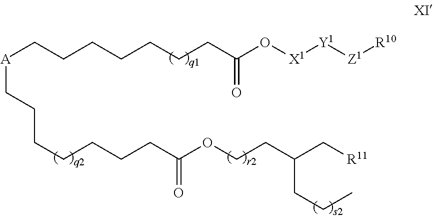

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XI′, wherein Z¹ is an optionally substituted C₅-C₁₂ bridged cycloalkylenyl.

In another embodiment, the disclosure provides a compound of Formula XI″:

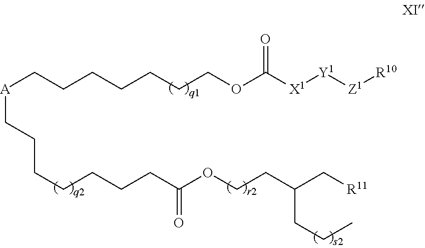

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC,
Formula ID, Formula I, or below.

In certain embodiments, the compound is a compound of Formula XI″, wherein Z¹ is an optionally substituted C₅-C₁₂ bridged cycloalkylenyl.

In another embodiment, the disclosure provides a compound of Formula XI″″:

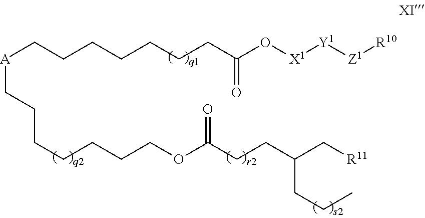

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XI″″ wherein Z¹ is an optionally substituted C₅-C₁₂ bridged cycloalkylenyl.

In another embodiment, the disclosure provides a compound of Formula XII:

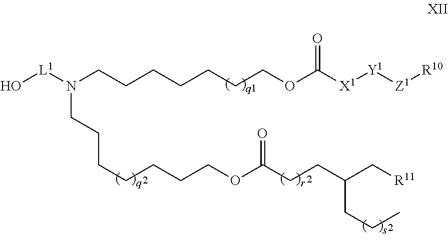

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula IC, Formula ID, Formula I, or below.

In certain embodiments, the compound is a compound of Formula XII, wherein Z¹ is an optionally substituted C₅-C₁₂ bridged cycloalkylenyl.

In another embodiment, the disclosure provides a compound of Formula XII′:

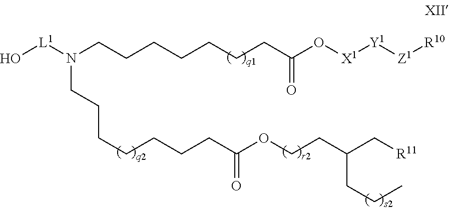

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula IC, Formula ID, Formula I, or below.

In certain embodiments, the compound is a compound of Formula XII′, wherein Z¹ is an optionally substituted C₅-C₁₂ bridged cycloalkylenyl.

In another embodiment, the disclosure provides a compound of Formula XII″:

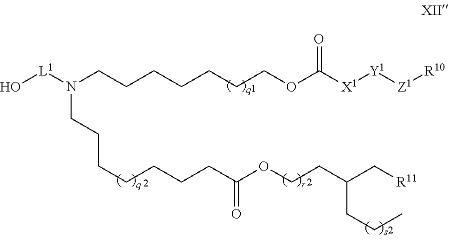

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula IC, Formula ID, Formula I or below.

In certain embodiments, the compound is a compound of Formula XII″, wherein Z¹ is an optionally substituted C₅-C₁₂ bridged cycloalkylenyl.

In another embodiment, the disclosure provides a compound of Formula XII″′:

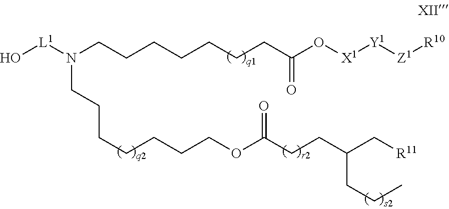

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula IC, Formula ID, Formula I or below.

In certain embodiments, the compound is a compound of Formula XII″″, wherein Z¹ is an optionally substituted C₅-C₁₂ bridged cycloalkylenyl.

In another embodiment, the disclosure provides a compound of Formula XIII:

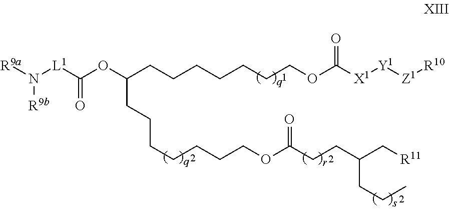

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula I or below.

In certain embodiments, the compound is a compound of Formula XIII, wherein Z¹ is an optionally substituted C₅-C₁₂ bridged cycloalkylenyl.

In another embodiment, the disclosure provides a compound of Formula XIII′:

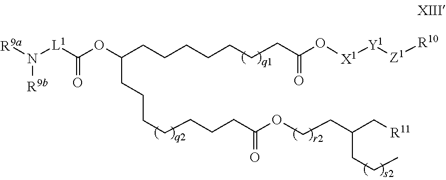

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R⁹%, R¹⁰ and R¹¹ are as defined herein in Formula IA, Formula IB, Formula IC, Formula ID, Formula I, or below.

In certain embodiments, the compound is a compound of Formula XIII′, wherein Z¹ is an optionally substituted C₅-C₁₂ bridged cycloalkylenyl.

In another embodiment, the disclosure provides a compound of Formula XIII″:

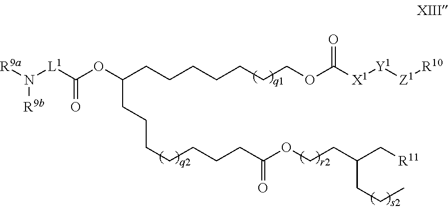

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In certain embodiments, the compound is a compound of Formula XIII″, wherein Z¹ is an optionally substituted C₅-C₁₂ bridged cycloalkylenyl.

In another embodiment, the disclosure provides a compound of Formula XIII″′:

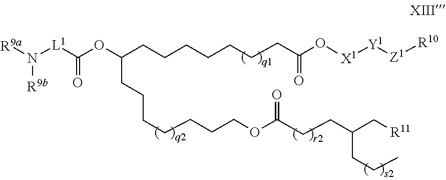

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R⁹, R¹⁰ and R¹¹ are as defined herein in Formula IA, Formula IB, Formula IC, Formula ID, Formula I or below.

In certain embodiments, the compound is a compound of Formula XIII″″, wherein Z¹ is an optionally substituted C₅-C₁₂ bridged cycloalkylenyl.

In another embodiment, the disclosure provides a compound of Formula XIV:

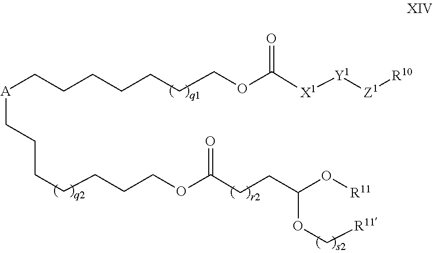

or a pharmaceutically acceptable salt or solvate thereof,
wherein
R¹¹′ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XIV, wherein Z¹ is an optionally substituted C₅-C₁₂ bridged cycloalkylenyl. In certain embodiments, Z¹ is not adamantyl.

In another embodiment, the disclosure provides a compound of Formula XIV′:

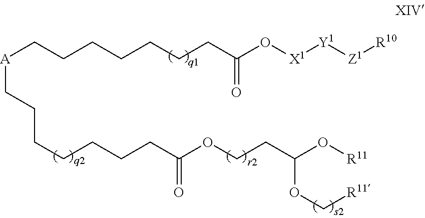

or a pharmaceutically acceptable salt or solvate thereof,
wherein
R¹¹′ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XIV′, wherein Z¹ is an optionally substituted C₅-C₁₂ bridged cycloalkylenyl. In certain embodiments, Z¹ is not adamantyl.

In another embodiment, the disclosure provides a compound of Formula XIV″:

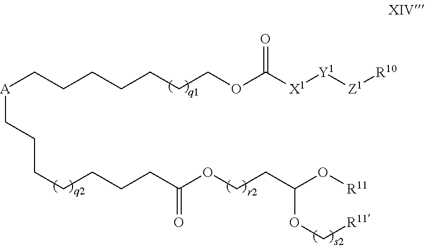

or a pharmaceutically acceptable salt or solvate thereof,
wherein
R¹¹′ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I or below.

In certain embodiments, the compound is a compound of Formula XIV″, wherein Z¹ is an optionally substituted C₅-C₁₂ bridged cycloalkylenyl. In certain embodiments, Z¹ is not adamantyl.

In another embodiment, the disclosure provides a compound of Formula XIV″″.

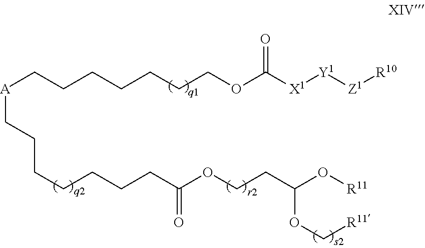

or a pharmaceutically acceptable salt or solvate thereof,
wherein
R¹¹′ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6, and
A, X¹, Y¹, Z¹, R¹⁰, and R¹¹ are as defined herein in Formula IA, Formula IB, Formula IC, Formula ID, Formula I or below.

In certain embodiments, the compound is a compound of Formula XIV″″ wherein Z¹ is an optionally substituted C₅-C₁₂ bridged cycloalkylenyl. In certain embodiments, Z¹ is not adamantyl.

In another embodiment, the disclosure provides a compound of Formula XV:

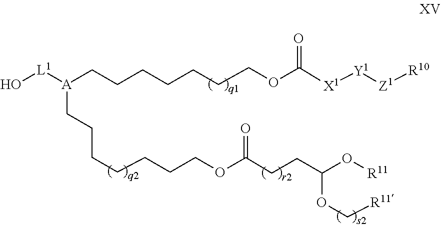

or a pharmaceutically acceptable salt or solvate thereof,
wherein
R¹¹′ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XV′:

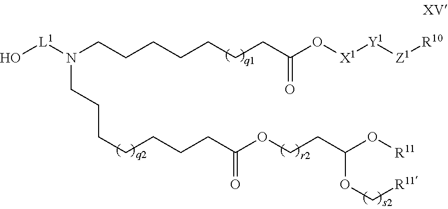

or a pharmaceutically acceptable salt or solvate thereof,
wherein
R¹¹′ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XV″:

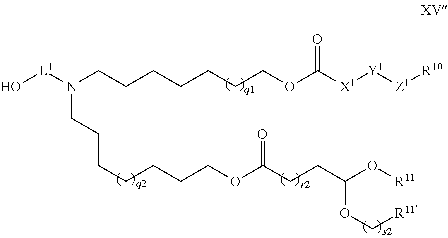

or a pharmaceutically acceptable salt or solvate thereof,
wherein
R′″ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XV″′:

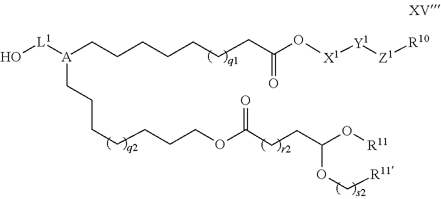

or a pharmaceutically acceptable salt or solvate thereof,
wherein
R¹¹′ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XVI:

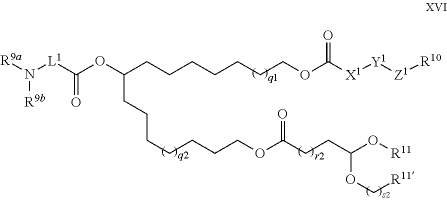

or a pharmaceutically acceptable salt or solvate thereof,
wherein
R¹¹′ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVI′:

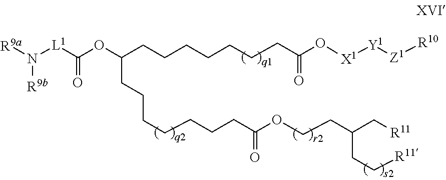

or a pharmaceutically acceptable salt or solvate thereof,
wherein
R¹¹′ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVI″:

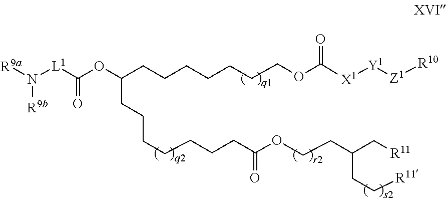

or a pharmaceutically acceptable salt or solvate thereof,
wherein
R¹¹′ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVI″″:

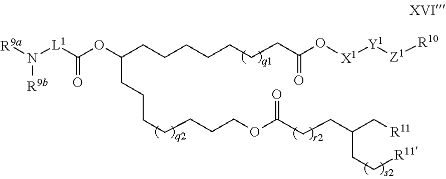

or a pharmaceutically acceptable salt or solvate thereof,
wherein
R¹¹′ is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and L¹, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVII:

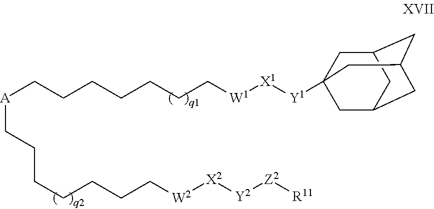

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula XVII, wherein one or more methylene linkages of X², Y², Z², and R¹¹, are not replaced with a group selected from —O—, —CH═CH—, —S— and C₃-C₆ cycloalkylenyl.

In another embodiment, the disclosure provides a compound of Formula XVIII:

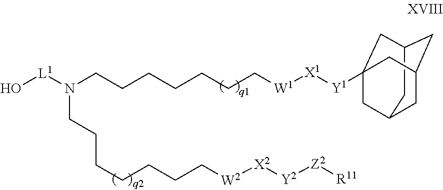

or a pharmaceutically acceptable salt or solvate thereof,
wherein
q¹ is 0, 1, 2, or 3;
q² is 0, 1, 2, or 3;
L¹, X¹, X², Y¹, Y², Z², an R¹¹ are as defined herein in Formula IA, Formula IB, Formula IC, Formula ID, Formula I, or below.

In certain embodiments, the compound is a compound of Formula XVIII, wherein one or more methylene linkages of X², Y², Z², and R¹¹, are not replaced with a group selected from —O—, —CH═CH—, —S— and C₃-C₆ cycloalkylenyl.

In another embodiment, the disclosure provides a compound of Formula XVIII′:

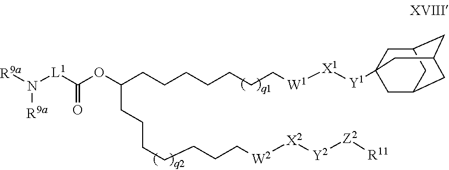

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula XIX:

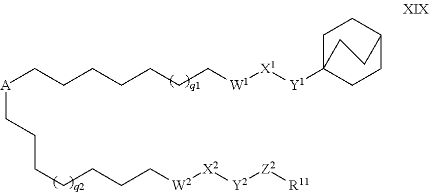

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula XX:

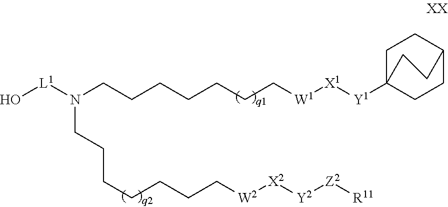

or a pharmaceutically acceptable salt or solvate thereof,
wherein
q¹ is 0, 1, 2, or 3;
q² is 0, 1, 2, or 3;
L¹, X¹, X², Y¹, Y², Z², an R¹¹ are as defined herein in Formula IA, Formula IB, Formula IC, Formula ID, Formula I, or below.

In another embodiment, the disclosure provides a compound of Formula XXI:

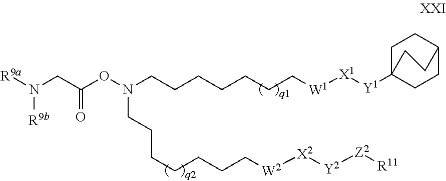

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

### L¹

In another embodiment, L¹ is selected from the group consisting of —CH₂CH₂—, —CH₂CH₂CH₂—, and —CH₂CH₂CH₂CH₂—. In another embodiment, L¹ is —CH₂CH₂—. In another embodiment, L¹ is —CH₂CH₂CH₂—. In another embodiment, L¹ is —CH₂CH₂CH₂CH₂—. In certain embodiments, L¹ is —(CH₂)₂₋₆—OC(═O)—. In some embodiments, L¹ is —(CH₂)₂—OC(═O)—

### R¹

In some embodiments, R¹ is

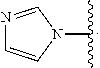 [SMILES: *n1ccnc1]

In another embodiment, R¹ is —OH. In some embodiments, R¹ is —N(R^{9a})(R^{9b}). In some embodiments, R¹ is —NMe₂. In some embodiments, R¹ is —NEt₂. In another embodiment, R¹ is

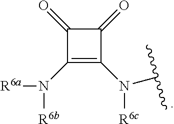

In another embodiment, R¹ is

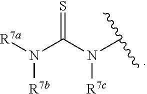

### L²

In another embodiment, L¹ is selected from the group consisting of —CH₂CH₂—, —CH₂CH₂CH₂—, and —CH₂CH₂CH₂CH₂—. In another embodiment, L² is —CH₂CH₂—. In another embodiment, L² is —CH₂CH₂CH₂—. In another embodiment, L² is —CH₂CH₂CH₂CH₂—.

### R⁸

In some embodiments, R⁸ is

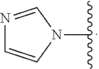 [SMILES: *n1ccnc1]

In another embodiment, R⁸ is —NR^{9a}R^{9b}. In some embodiments, R⁸ is —NMe₂. In some embodiments, R⁸ is —NEt₂. In another embodiment, R⁸ is —OH.
R^{9a}, R^{9b}

In another embodiment, R^{9a} and Rob are independently selected from the group consisting of hydrogen and C₁-C₄ alkyl. In another embodiment, R^{9a} and R^{9b} are each methyl. In another embodiment, R^{9a} and R^{9b} are each ethyl.

### R′

In another embodiment, R′ is hydrogen. In some embodiments, R′ is C₁-C₆ alkyl.

### Q¹

In another embodiment, Q¹ is straight chain C₁-C₂₀ alkylenyl. In another embodiment, Q¹ is straight chain C₁-C₁₀ alkylenyl. In another embodiment, Q¹ is C₁-C₁₀ alkylenyl. In another embodiment, Q¹ is C₂-C₅ alkylenyl. Q¹ is C₆-C₉ alkylenyl. In another embodiment, Q¹ is selected from the group consisting of —CH₂CH₂—, —CH₂CH₂CH₂—, —CH₂ (CH₂)₂CH₂—, —CH₂ (CH₂)₃CH₂—, —CH₂ (CH₂)₄CH₂—, —CH₂ (CH₂)₅CH₂—, —CH₂ (CH₂)₆CH₂—, —CH₂ (CH₂)—CH₂—, and —CH₂ (CH₂)₈CH₂—. In another embodiment, Q¹ is —CH₂CH₂—. In another embodiment, Q¹ is —CH₂CH₂CH₂—. In another embodiment, Q¹ is —CH₂ (CH₂)₂CH₂—. In another embodiment, Q¹ is —CH₂ (CH₂)₃CH₂—. In another embodiment, Q¹ is —CH₂CH₂—. In another embodiment, Q¹ is —CH₂ (CH₂)₄CH₂—. In another embodiment, Q¹ is —CH₂ (CH₂)₅CH₂—. In another embodiment, Q¹ is —CH₂ (CH₂)₆CH₂—. In another embodiment, Q¹ is —CH₂ (CH₂)₇CH₂—. In another embodiment, Q¹ is —CH₂ (CH₂)₈.CH₂—.

### W¹

In another embodiment, W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—. In another embodiment, W¹ is —C(═O)O—. In another embodiment, W¹ is —OC(═O)—. In another embodiment, W¹ is —C(═O)N(R^{12a})—. In another embodiment, W¹ is —N(R^{12a})C(═O)—. In another embodiment, W¹ is —OC(═O)N(R^{12a})—. In another embodiment, W¹ is —N(R¹²a)C(═O)O—. In another embodiment, W¹ is —OC(═O)O—.

### X¹

In another embodiment, X² is optionally substituted C₁-C₁₅ alkylenyl. In another embodiment, X² is branched C₁-C₁₅ alkylenyl. In another embodiment, X¹ is a bond or C₁-C₁₅ alkylenyl. In another embodiment, X¹ is a bond. In another embodiment, X¹ is C₂-C₅ alkylenyl. In another embodiment, X¹ is C₆-C₉ alkylenyl. In another embodiment, X¹ is —CH₂—. In another embodiment, X² is —CH₂CH₂—. In another embodiment, X² is —CH₂CH₂CH₂—. In another embodiment, X² is —CH₂CH₂CH₂CH₂—. In another embodiment, X² is —CH₂CH₂CH₂CH₂CH₂—.

### Y¹

In another embodiment, Y¹ is selected from the group consisting of —(CH₂)ₘ—, —O—, —S—, and —S—S—. In another embodiment, Y¹ is —(CH₂)ₘ—. In some embodiments, Y¹ is —O—. In some embodiments, Y¹ is —S—. In another embodiment, Y¹ is —CH₂—. In another embodiment, Y² is —CH₂CH₂—.

m

In another embodiment, m is 0. In another embodiment, m is 1. In another embodiment, m is 2. In another embodiment, m is 3. In another embodiment, m is 4. In another embodiment, m is 5. In another embodiment, m is 6.

n

In another embodiment, n is 0. In another embodiment, n is 1. In another embodiment, n is 2. In another embodiment, n is 3. In another embodiment, n is 4. In another embodiment, n is 5. In another embodiment, n is 6.

p

In another embodiment, p is 0. In another embodiment, p is 1.

### Z¹

In another embodiment, Z¹ is selected from the group consisting of C₄-C₁₂ cycloalkylenyl,

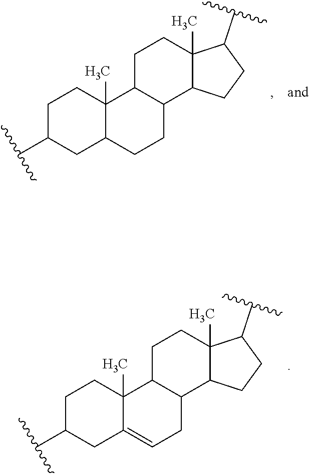 [SMILES: *C1CCC2(C)C(CCC3C4CCC(*)C4(C)CCC32)C1 ; *C1CCC2(C)C(=CCC3C2CCC2(C)C(*)CCC32)C1]

In certain embodiments, Z¹ is optionally substituted.

In another embodiment, Z¹ is

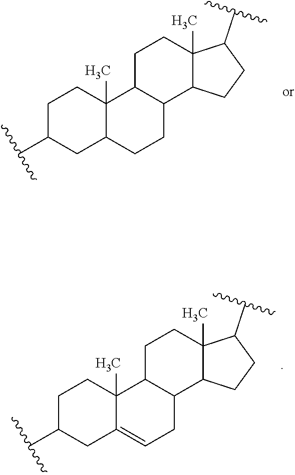 [SMILES: *C1CCC2(C)C(CCC3C4CCC(*)C4(C)CCC32)C1 ; *C1CCC2(C)C(=CCC3C2CCC2(C)C(*)CCC32)C1]

In another embodiment, Z¹ is C₄-C₁₂ cycloalkylenyl. In another embodiment, Z¹ is a monocyclic C₄-C₈ cycloalkylenyl. In another embodiment, Z¹ is a monocyclic C₄-C₆ cycloalkylenyl. In another embodiment, Z¹ is a monocyclic C₄ cycloalkylenyl. In another embodiment, Z¹ is a monocyclic C₅ cycloalkylenyl. In another embodiment, Z¹ is a monocyclic C₆ cycloalkylenyl.

In another embodiment, Z¹ is an optionally substituted bridged bicyclic or multicyclic cycloalkylenyl. In some embodiments, Z¹ is optionally substituted C₅-C₁₂ bridged cycloalkylenyl. In some embodiments, Z¹ is optionally substituted C₆-C₁₀ bridged cycloalkylenyl. In some embodiments, Z¹ is a optionally substituted C₅-C₁₀ bridged cycloalkylenyl. selected from the group consisting of adamantyl, cubanyl, bicyclo[2.2.1]heptyl, bicyclo[2.2.2]octyl, bicyclo[1.1.1]pentyl, bicyclo[3.2.1]octyl, and bicyclo[3.1.1]heptyl.

In another embodiment, Z¹ is selected from the group consisting of:

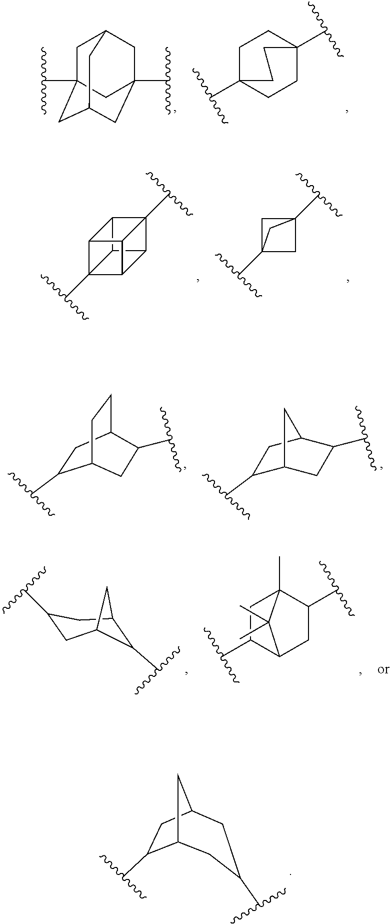

In another embodiment, Z¹ is selected from the group consisting of:

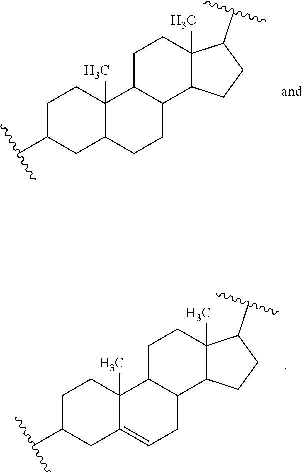 [SMILES: *C1CCC2(C)C(CCC3C4CCC(*)C4(C)CCC32)C1 ; *C1CCC2(C)C(=CCC3C2CCC2(C)C(*)CCC32)C1]

### R¹⁰

In another embodiment, R¹⁰ is hydrogen.

In another embodiment, R¹⁰ is C₁-C₁₀ alkyl. In another embodiment, R¹⁰ is C₃-C₇ alkyl. In another embodiment, R¹⁰ is C₄-C₆ alkyl. In another embodiment, R¹⁰ is C₄. In another embodiment, R¹⁰ is C₅. In another embodiment, R¹⁰ is C₆.

In another embodiment, R¹⁰ is C₂-C₁₂ alkenyl. In another embodiment, R¹⁰ is C₆-C₁₂ alkenyl. In another embodiment, R¹⁰ is C₂-C₈ alkenyl.

### R¹¹

In another embodiment, R¹¹ is C₁-C₁₀ alkyl. In another embodiment, R¹¹ is optionally substituted C₁-C₂₀ alkyl. In another embodiment, R¹¹ is optionally substituted branched C₁-C₂₀ alkyl. In another embodiment, R¹¹ is optionally substituted C₁-C₁₅ alkyl. In another embodiment, R¹¹ is optionally substituted C₁-C₁₅ branched alkyl. In another embodiment, R¹¹ is optionally substituted C₁₀-C₁₅ alkyl. In another embodiment, R¹¹ is optionally substituted C₁₀-C₁₅ branched alkyl. In another embodiment, R¹¹ is selected from the group consisting of —CH₃, —CH₂CH₃, and —CH₂CH₂CH₃. In another embodiment, R¹¹ is selected from the group consisting of —CH₂ (CH₂)₂CH₃, —CH₂ (CH₂)₃CH₃, —CH₂ (CH₂)₄CH₃, —CH₂ (CH₂)₅CH₃, —CH₂ (CH₂)₆CH₃, —CH₂ (CH₂)₇CH₃, and —CH₂ (CH₂)₈CH₃. In another embodiment, R¹¹ is —CH₃. In another embodiment, R¹¹ is —CH₂CH₃. In another embodiment, R¹¹ is —CH₂CH₂CH₃. In another embodiment, R¹¹ is —CH₂ (CH₂)₂CH₃. In another embodiment, R¹¹ is —CH₂ (CH₂)₃CH₃. In another embodiment, R¹¹ is —CH₂ (CH₂)₄CH₃. In another embodiment, R¹¹ is —CH₂ (CH₂)₅CH₃. In another embodiment, R¹¹ is CH₂ (CH₂)₆CH₃. In another embodiment, R¹¹ is —CH₂ (CH₂)₇CH₃. In another embodiment, R¹¹ is —CH₂ (CH₂)₈CH₃.

In another embodiment, R¹¹ is C₂-C₁₀ alkenyl. In another embodiment, R¹¹ is C₂-C₁₂ alkenyl. In another embodiment, R¹¹ is C₆-C₁₂ alkenyl. In another embodiment, R¹¹ is C₂-C₈ alkenyl.

In another embodiment, the disclosure provides a compound of any one of Formulae IA, IB, IC, or I-XXI or a pharmaceutically acceptable salt or solvate thereof, wherein R¹¹ is hydrogen.

### Q²

In another embodiment, Q² is straight chain C₁-C₂₀ alkylenyl. In another embodiment, Q² is straight chain C₁-C₁₀ alkylenyl. In another embodiment, Q² is C₂-C₁₀ alkylenyl. In another embodiment, Q² is selected from the group consisting of —CH₂CH₂—, —CH₂CH₂CH₂—, —CH₂ (CH₂)₂CH₂—, —CH₂ (CH₂)₃CH₂—, —CH₂ (CH₂)₄CH₂—, —CH₂ (CH₂)₅CH₂—, —CH₂ (CH₂)₆CH₂—, —CH₂ (CH₂)₇CH₂—, and —CH₂ (CH₂)₈. CH₂—. In another embodiment, Q² is —CH₂CH₂—. In another embodiment, Q² is —CH₂CH₂CH₂—. In another embodiment, Q² is —CH₂ (CH₂)₃CH₂—. In another embodiment, Q² is —CH₂ (CH₂)₄CH₂—. In another embodiment, Q² is —CH₂ (CH₂)₅CH₂—. In another embodiment, Q² is —CH₂ (CH₂)₆CH₂—. In another embodiment, Q² is —CH₂ (CH₂)₇CH₂—. In another embodiment, Q² is —CH₂ (CH₂)₈.CH₂—.

### W²

In another embodiment, W² is selected from the group consisting of —C(═O)O- and —OC(═O)—. In another embodiment, W² is —C(═O)O—. In another embodiment, W² is —OC(═O)—.

### X²

In another embodiment, X² is optionally substituted C₁-C₁₅ alkylenyl. In another embodiment, X² is C₁-C₁₅ branched alkylenyl. In another embodiment, X² is C₁-C₆ alkylenyl or a bond. In another embodiment, X² is C₂-C₄ alkylenyl. In another embodiment, X² is C₃-C₅ alkylenyl. In another embodiment, X² is selected from the group consisting of —CH₂CH₂—, —CH₂CH₂CH₂—, —CH₂ (CH₂)₂CH₂—, —CH₂ (CH₂)₃CH₂—, and —CH₂ (CH₂)₄CH₂—. In another embodiment, X² is —CH₂—. In another embodiment, X² is a bond. In another embodiment, X² is branched C₁-C₁₅ alkylenyl, wherein one or more methylene linkages of X² are optionally and independently replaced with a group selected from —O—, —CH═CH—, —S— and C₃-C₆ cycloalkylenyl.

### Y²

In another embodiment, Y² is selected from the group consisting of —(CH₂)ₘ- and —S—. In another embodiment, Y² is —(CH₂)ₘ—. In another embodiment, Y² is —S—.

### Z²

In another embodiment, Z² is —(CH₂)ₚ—. In another embodiment, Z² is —CH₂—. In another embodiment, Z² is —CH₂CH₂—. In another embodiment, Z² is C₄-C₁₂ cycloalkylenyl. In another embodiment, Z² is a monocyclic C₄-C₈ cycloalkylenyl. In certain embodiments, Z² is optionally substituted.

In another embodiment, Z² is an optionally substituted bridged bicyclic or multicyclic cycloalkylenyl. In some embodiments, Z² is optionally substituted C₅-C₁₂ bridged cycloalkylenyl. In some embodiments, Z² is optionally substituted C₆-C₁₀ bridged cycloalkylenyl. In some embodiments, Z² is a optionally substituted C₅-C₁₀ bridged cycloalkylenyl. selected from the group consisting of adamantyl, cubanyl, bicyclo[2.2.1]heptyl, bicyclo[2.2.2]octyl, bicyclo[1.1.1]pentyl, bicyclo[3.2.1]octyl, and bicyclo[3.1.1]heptyl.

In another embodiment, Z² is selected from the group consisting of:

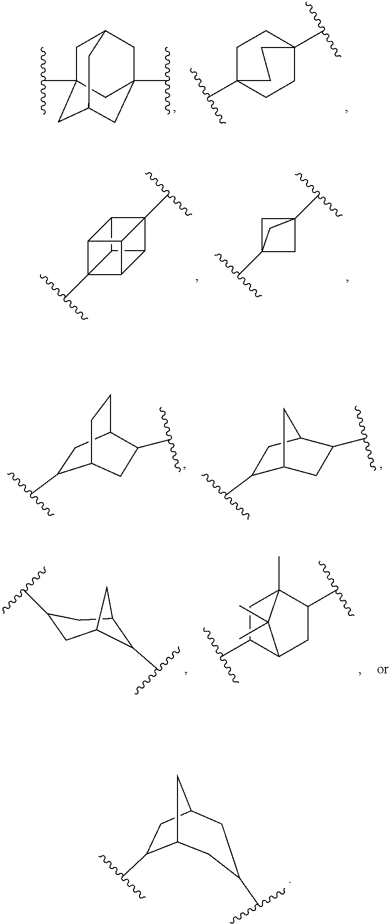

In another embodiment, Z² is selected from the group consisting of:

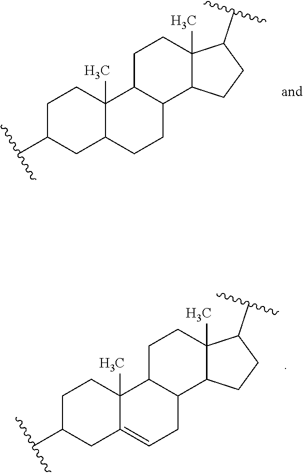 [SMILES: *C1CCC2(C)C(CCC3C4CCC(*)C4(C)CCC32)C1 ; *C1CCC2(C)C(=CCC3C2CCC2(C)C(*)CCC32)C1]

In another embodiment, the disclosure provides a compound selected from any one of more of the compounds of Table (III), or a pharmaceutically acceptable salt or solvate thereof.

TABLE (III)
Non-Limiting Examples of Ionizable Lipids with a Constrained Arm

| Compound No. | Structure |
|---|---|
| C1 | 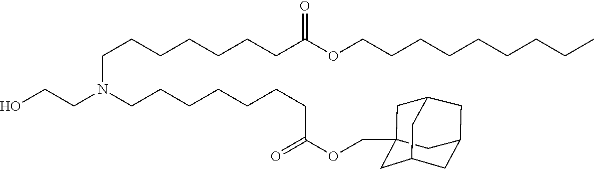 [SMILES: CCCCCCCCCOC(=O)CCCCCCCN(CCO)CCCCCCCC(=O)OCC12CC3CC(CC(C3)C1)C2] |
| C2 | 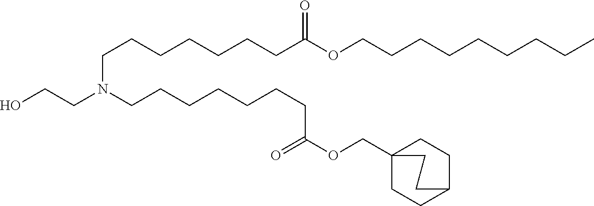 [SMILES: CCCCCCCCCOC(=O)CCCCCCCN(CCO)CCCCCCCC(=O)OCC12CCC(CC1)CC2] |
| C3 | 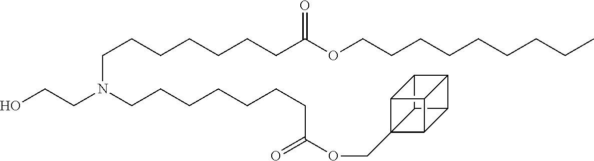 [SMILES: CCCCCCCCCOC(=O)CCCCCCCN(CCO)CCCCCCCC(=O)OCC12C3C4C5C3C1C5C42] |
| C4 | 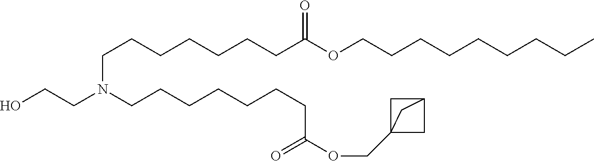 [SMILES: CCCCCCCCCOC(=O)CCCCCCCN(CCO)CCCCCCCC(=O)OCC12CC(C1)C2] |
| C5 | 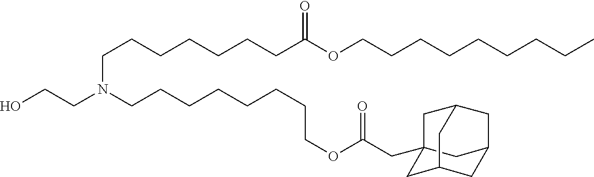 [SMILES: CCCCCCCCCOC(=O)CCCCCCCN(CCO)CCCCCCCCOC(=O)CC12CC3CC(CC(C3)C1)C2] |
| C6 | 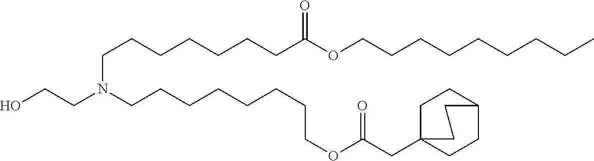 [SMILES: CCCCCCCCCOC(=O)CCCCCCCN(CCO)CCCCCCCCOC(=O)CC12CCC(CC1)CC2] |
| C7 | 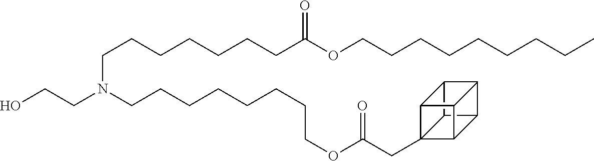 [SMILES: CCCCCCCCCOC(=O)CCCCCCCN(CCO)CCCCCCCCOC(=O)CC12C3C4C5C3C1C5C42] |
| C8 | 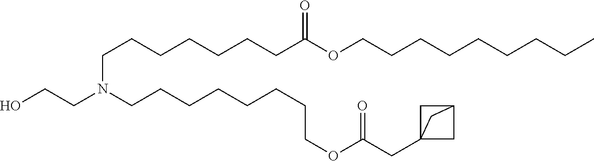 [SMILES: CCCCCCCCCOC(=O)CCCCCCCN(CCO)CCCCCCCCOC(=O)CC12CC(C1)C2] |
| C9 | 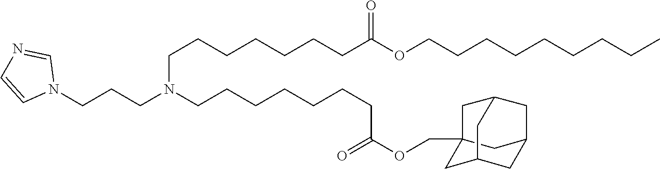 [SMILES: CCCCCCCCCOC(=O)CCCCCCCN(CCCCCCCC(=O)OCC12CC3CC(CC(C3)C1)C2)CCCn1ccnc1] |
| C10 | 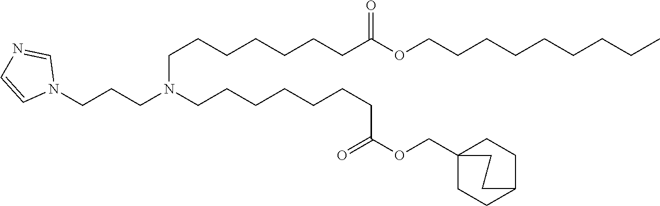 [SMILES: CCCCCCCCCOC(=O)CCCCCCCN(CCCCCCCC(=O)OCC12CCC(CC1)CC2)CCCn1ccnc1] |
| C11 | 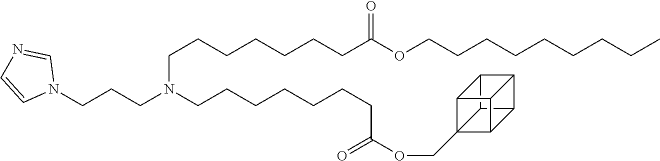 [SMILES: CCCCCCCCCOC(=O)CCCCCCCN(CCCCCCCC(=O)OCC12C3C4C5C3C1C5C42)CCCn1ccnc1] |
| C12 | 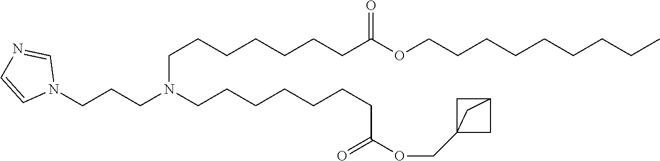 [SMILES: CCCCCCCCCOC(=O)CCCCCCCN(CCCCCCCC(=O)OCC12CC(C1)C2)CCCn1ccnc1] |
| C13 | 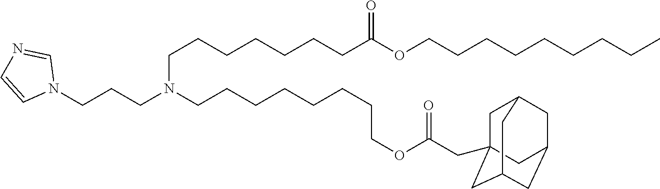 [SMILES: CCCCCCCCCOC(=O)CCCCCCCN(CCCCCCCCOC(=O)CC12CC3CC(CC(C3)C1)C2)CCCn1ccnc1] |
| C14 | 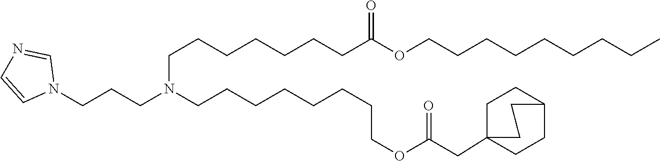 [SMILES: CCCCCCCCCOC(=O)CCCCCCCN(CCCCCCCCOC(=O)CC12CCC(CC1)CC2)CCCn1ccnc1] |
| C15 | 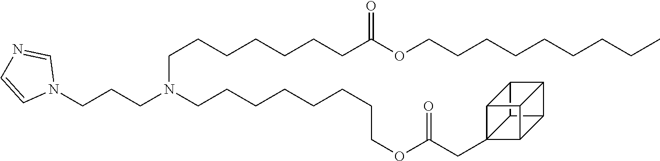 [SMILES: CCCCCCCCCOC(=O)CCCCCCCN(CCCCCCCCOC(=O)CC12C3C4C5C3C1C5C42)CCCn1ccnc1] |
| C16 | 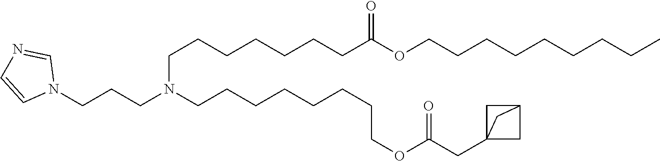 [SMILES: CCCCCCCCCOC(=O)CCCCCCCN(CCCCCCCCOC(=O)CC12CC(C1)C2)CCCn1ccnc1] |
| C17 | 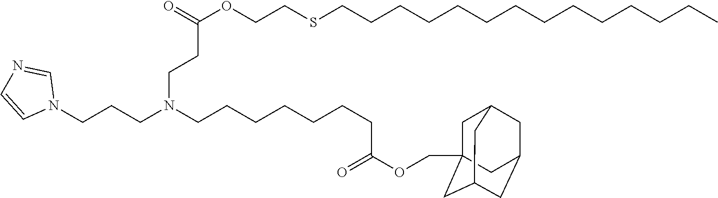 [SMILES: CCCCCCCCCCCCCCSCCOC(=O)CCN(CCCCCCCC(=O)OCC12CC3CC(CC(C3)C1)C2)CCCn1ccnc1] |
| C18 | 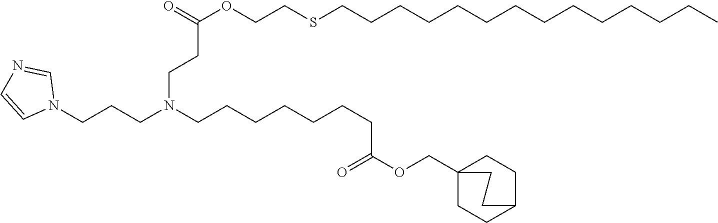 [SMILES: CCCCCCCCCCCCCCSCCOC(=O)CCN(CCCCCCCC(=O)OCC12CCC(CC1)CC2)CCCn1ccnc1] |
| C19 | 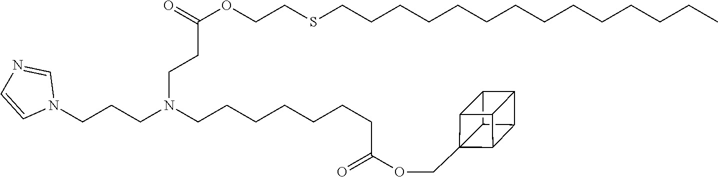 [SMILES: CCCCCCCCCCCCCCSCCOC(=O)CCN(CCCCCCCC(=O)OCC12C3C4C5C3C1C5C42)CCCn1ccnc1] |
| C20 | 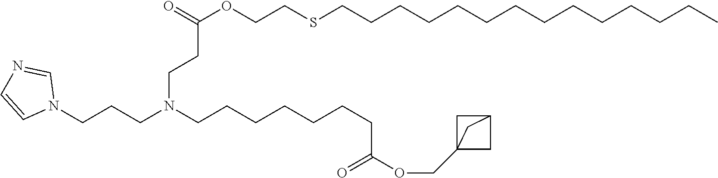 [SMILES: CCCCCCCCCCCCCCSCCOC(=O)CCN(CCCCCCCC(=O)OCC12CC(C1)C2)CCCn1ccnc1] |
| C21 | 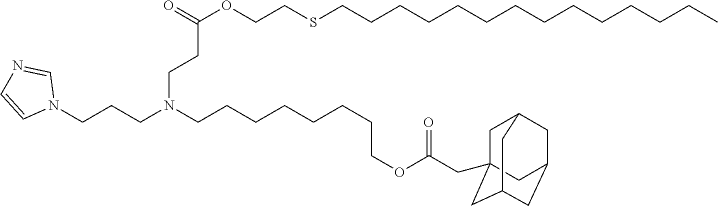 [SMILES: CCCCCCCCCCCCCCSCCOC(=O)CCN(CCCCCCCCOC(=O)CC12CC3CC(CC(C3)C1)C2)CCCn1ccnc1] |
| C22 | 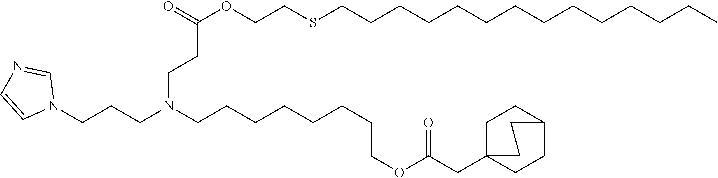 [SMILES: CCCCCCCCCCCCCCSCCOC(=O)CCN(CCCCCCCCOC(=O)CC12CCC(CC1)CC2)CCCn1ccnc1] |
| C23 | 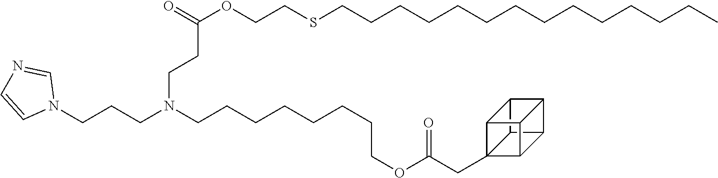 [SMILES: CCCCCCCCCCCCCCSCCOC(=O)CCN(CCCCCCCCOC(=O)CC12C3C4C5C3C1C5C42)CCCn1ccnc1] |
| C24 | 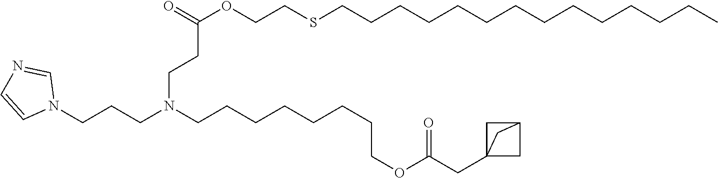 [SMILES: CCCCCCCCCCCCCCSCCOC(=O)CCN(CCCCCCCCOC(=O)CC12CC(C1)C2)CCCn1ccnc1] |
| C25 | 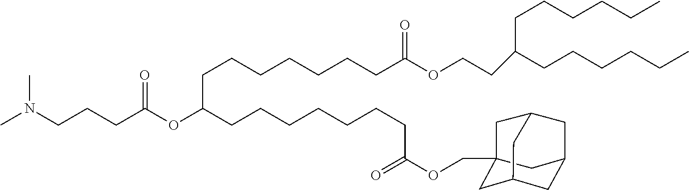 [SMILES: CCCCCCC(CCCCCC)CCOC(=O)CCCCCCCC(CCCCCCCC(=O)OCC12CC3CC(CC(C3)C1)C2)OC(=O)CCCN(C)C] |
| C26 | 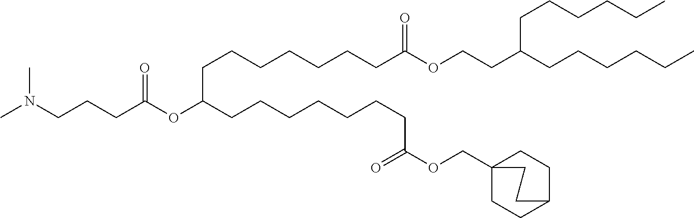 [SMILES: CCCCCCC(CCCCCC)CCOC(=O)CCCCCCCC(CCCCCCCC(=O)OCC12CCC(CC1)CC2)OC(=O)CCCN(C)C] |
| C27 | 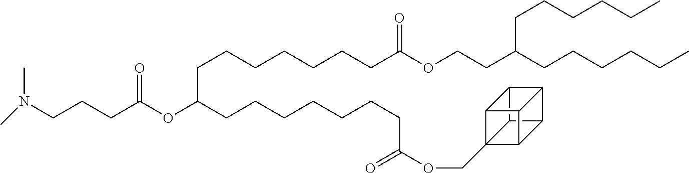 [SMILES: CCCCCCC(CCCCCC)CCOC(=O)CCCCCCCC(CCCCCCCC(=O)OCC12C3C4C5C3C1C5C42)OC(=O)CCCN(C)C] |
| C28 | 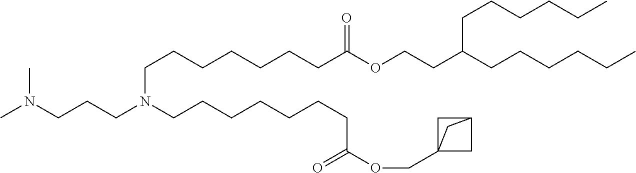 [SMILES: CCCCCCC(CCCCCC)CCOC(=O)CCCCCCCN(CCCCCCCC(=O)OCC12CC(C1)C2)CCCN(C)C] |
| C29 | 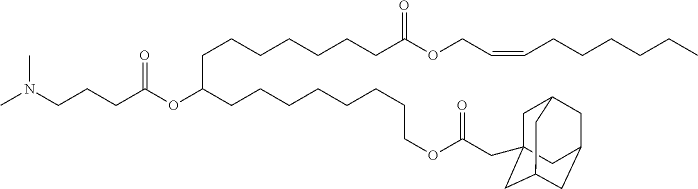 [SMILES: CCCCCC/C=C\COC(=O)CCCCCCCC(CCCCCCCCOC(=O)CC12CC3CC(CC(C3)C1)C2)OC(=O)CCCN(C)C] |
| C30 | 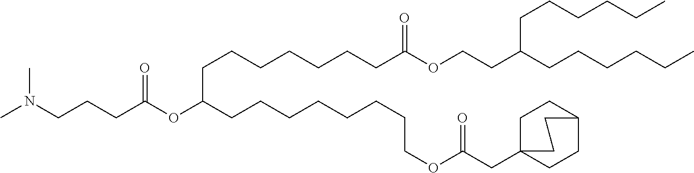 [SMILES: CCCCCCC(CCCCCC)CCOC(=O)CCCCCCCC(CCCCCCCCOC(=O)CC12CCC(CC1)CC2)OC(=O)CCCN(C)C] |
| C31 | 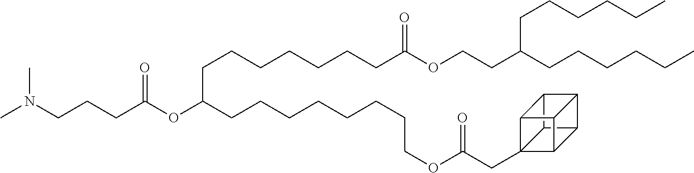 [SMILES: CCCCCCC(CCCCCC)CCOC(=O)CCCCCCCC(CCCCCCCCOC(=O)CC12C3C4C5C3C1C5C42)OC(=O)CCCN(C)C] |
| C32 | 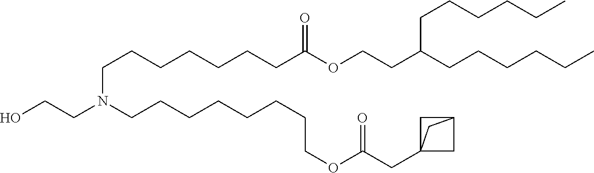 [SMILES: CCCCCCC(CCCCCC)CCOC(=O)CCCCCCCN(CCO)CCCCCCCCOC(=O)CC12CC(C1)C2] |
| C33 | 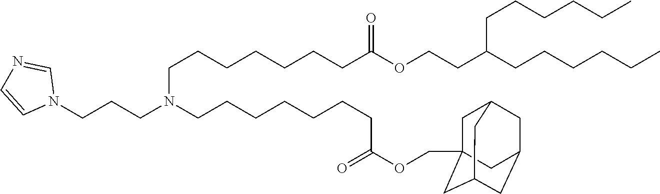 [SMILES: CCCCCCC(CCCCCC)CCOC(=O)CCCCCCCN(CCCCCCCC(=O)OCC12CC3CC(CC(C3)C1)C2)CCCn1ccnc1] |
| C34 | 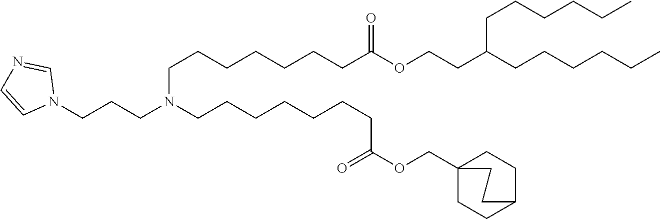 [SMILES: CCCCCCC(CCCCCC)CCOC(=O)CCCCCCCN(CCCCCCCC(=O)OCC12CCC(CC1)CC2)CCCn1ccnc1] |
| C35 | 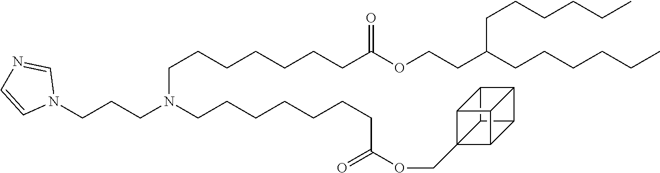 [SMILES: CCCCCCC(CCCCCC)CCOC(=O)CCCCCCCN(CCCCCCCC(=O)OCC12C3C4C5C3C1C5C42)CCCn1ccnc1] |
| C36 | 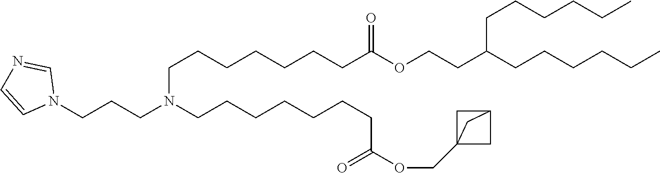 [SMILES: CCCCCCC(CCCCCC)CCOC(=O)CCCCCCCN(CCCCCCCC(=O)OCC12CC(C1)C2)CCCn1ccnc1] |
| C37 | 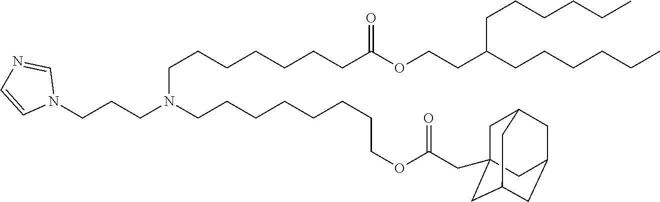 [SMILES: CCCCCCC(CCCCCC)CCOC(=O)CCCCCCCN(CCCCCCCCOC(=O)CC12CC3CC(CC(C3)C1)C2)CCCn1ccnc1] |
| C38 | 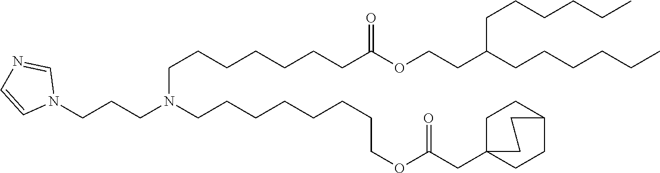 [SMILES: CCCCCCC(CCCCCC)CCOC(=O)CCCCCCCN(CCCCCCCCOC(=O)CC12CCC(CC1)CC2)CCCn1ccnc1] |
| C39 | 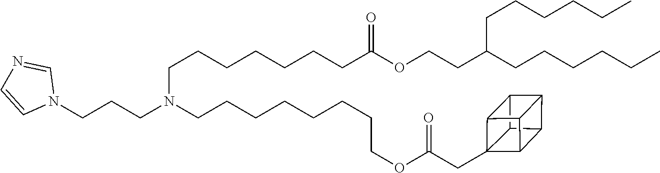 [SMILES: CCCCCCC(CCCCCC)CCOC(=O)CCCCCCCN(CCCCCCCCOC(=O)CC12C3C4C5C3C1C5C42)CCCn1ccnc1] |
| C40 | 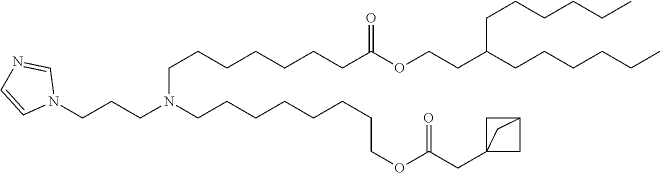 [SMILES: CCCCCCC(CCCCCC)CCOC(=O)CCCCCCCN(CCCCCCCCOC(=O)CC12CC(C1)C2)CCCn1ccnc1] |
| C41 | 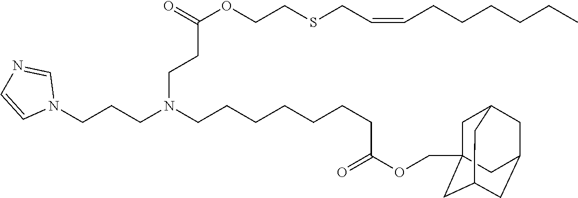 [SMILES: CCCCCC/C=C\CSCCOC(=O)CCN(CCCCCCCC(=O)OCC12CC3CC(CC(C3)C1)C2)CCCn1ccnc1] |
| C42 | 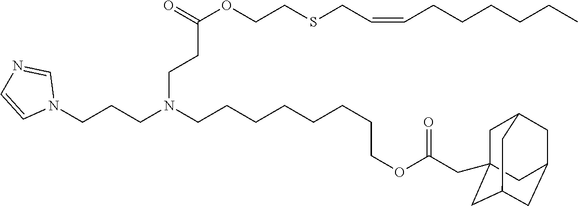 [SMILES: CCCCCC/C=C\CSCCOC(=O)CCN(CCCCCCCCOC(=O)CC12CC3CC(CC(C3)C1)C2)CCCn1ccnc1] |
| C43 | 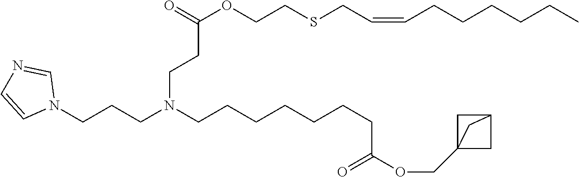 [SMILES: CCCCCC/C=C\CSCCOC(=O)CCN(CCCCCCCC(=O)OCC12CC(C1)C2)CCCn1ccnc1] |
| C44 | 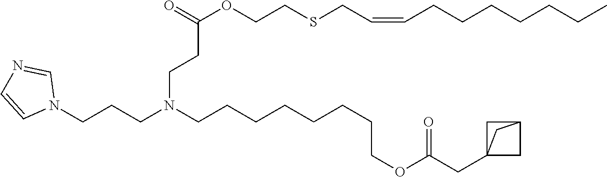 [SMILES: CCCCCCC/C=C\CSCCOC(=O)CCN(CCCCCCCCOC(=O)CC12CC(C1)C2)CCCn1ccnc1] |
| C45 | 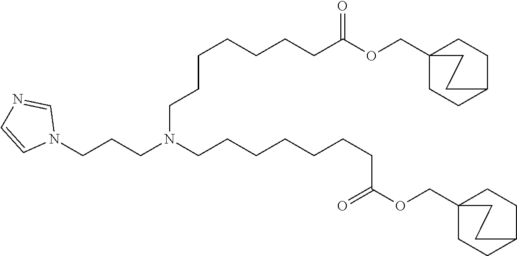 [SMILES: O=C(CCCCCCCN(CCCCCCCC(=O)OCC12CCC(CC1)CC2)CCCn1ccnc1)OCC12CCC(CC1)CC2] |
| C46 | 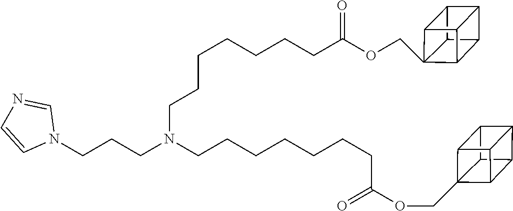 [SMILES: O=C(CCCCCCCN(CCCCCCCC(=O)OCC12C3C4C5C3C1C5C42)CCCn1ccnc1)OCC12C3C4C5C3C1C5C42] |
| C47 | 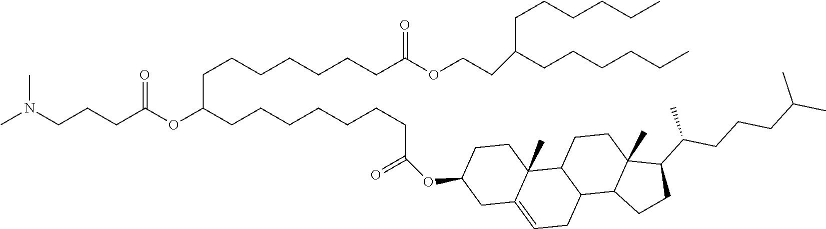 |
| C48 | 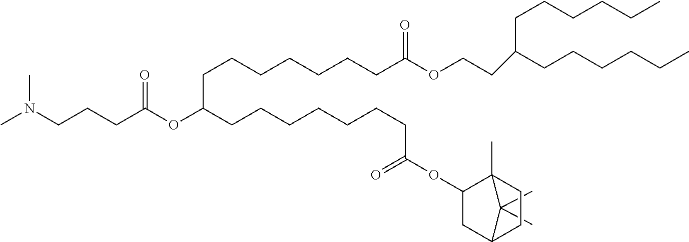 [SMILES: CCCCCCC(CCCCCC)CCOC(=O)CCCCCCCC(CCCCCCCC(=O)OC1CC2CCC1(C)C2(C)C)OC(=O)CCCN(C)C] |
| C49 | 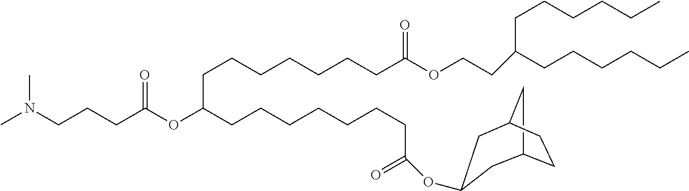 [SMILES: CCCCCCC(CCCCCC)CCOC(=O)CCCCCCCC(CCCCCCCC(=O)OC1CC2CCC(C2)C1)OC(=O)CCCN(C)C] |
| C50 | 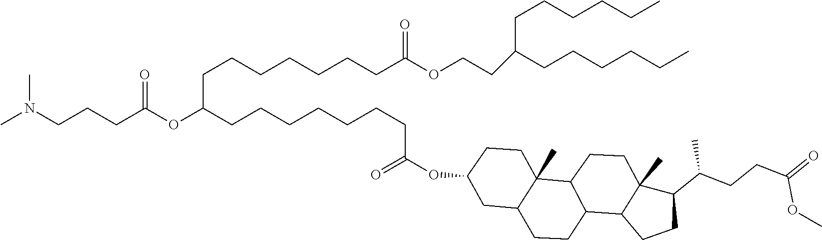 |
| C51 | 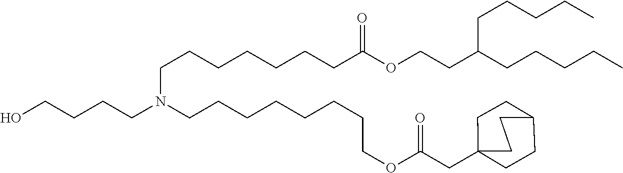 [SMILES: CCCCCC(CCCCC)CCOC(=O)CCCCCCCN(CCCCO)CCCCCCCCOC(=O)CC12CCC(CC1)CC2] |
| C52 | 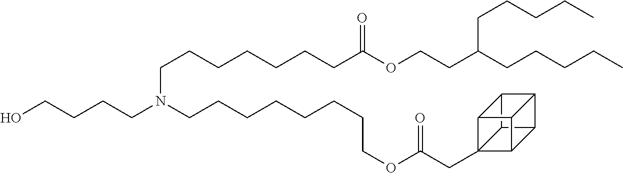 [SMILES: CCCCCC(CCCCC)CCOC(=O)CCCCCCCN(CCCCO)CCCCCCCCOC(=O)CC12C3C4C5C3C1C5C42] |
| C53 | 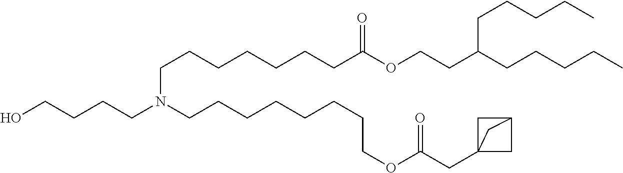 [SMILES: CCCCCC(CCCCC)CCOC(=O)CCCCCCCN(CCCCO)CCCCCCCCOC(=O)CC12CC(C1)C2] |
| C54 | 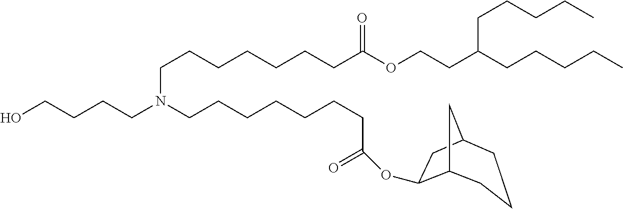 [SMILES: CCCCCC(CCCCC)CCOC(=O)CCCCCCCN(CCCCO)CCCCCCCC(=O)OC1CC2CCCC1C2] |
| C55 | 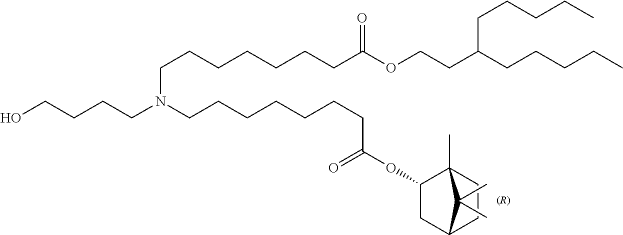 |
| C56 | 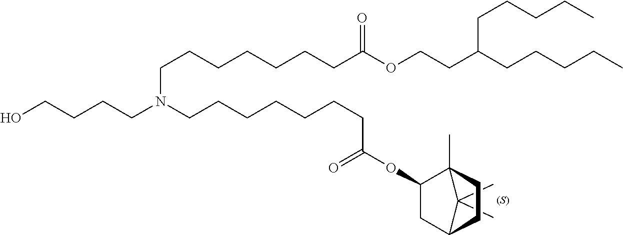 |
| C57 | 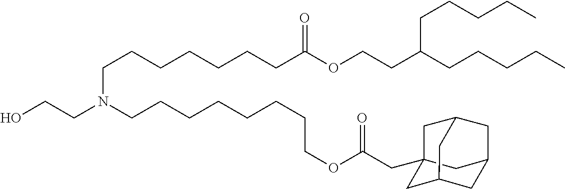 [SMILES: CCCCCC(CCCCC)CCOC(=O)CCCCCCCN(CCO)CCCCCCCCOC(=O)CC12CC3CC(CC(C3)C1)C2] |
| C58 | 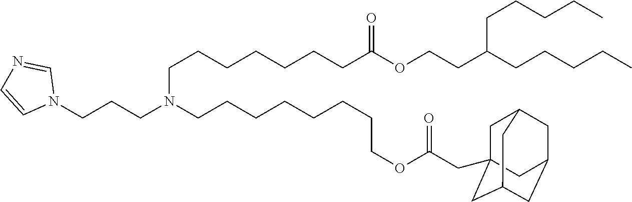 [SMILES: CCCCCC(CCCCC)CCOC(=O)CCCCCCCN(CCCCCCCCOC(=O)CC12CC3CC(CC(C3)C1)C2)CCCn1ccnc1] |
| C59 | 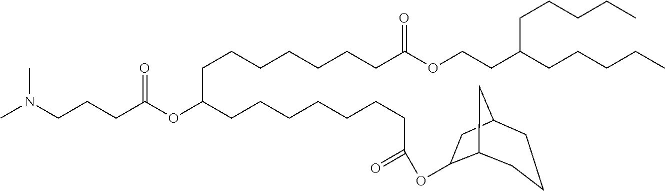 [SMILES: CCCCCC(CCCCC)CCOC(=O)CCCCCCCC(CCCCCCCC(=O)OC1CC2CCCC1C2)OC(=O)CCCN(C)C] |
| C60 | 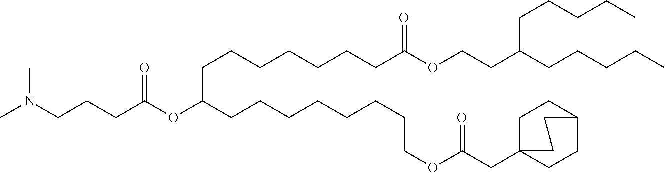 [SMILES: CCCCCC(CCCCC)CCOC(=O)CCCCCCCC(CCCCCCCCOC(=O)CC12CCC(CC1)CC2)OC(=O)CCCN(C)C] |
| C61 | 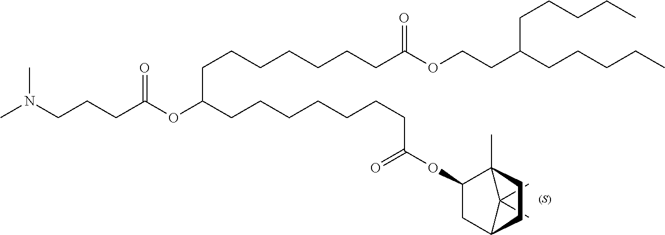 |
| C62 | 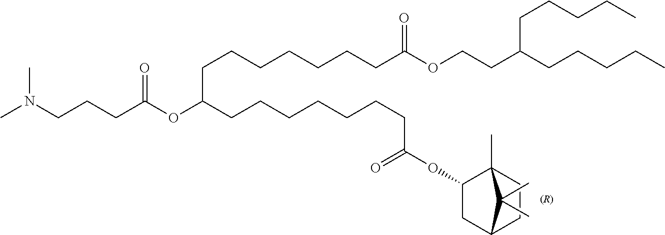 |
| C63 | 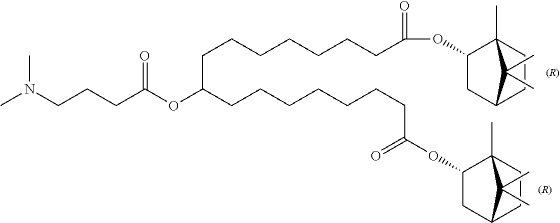 |
| C64 | 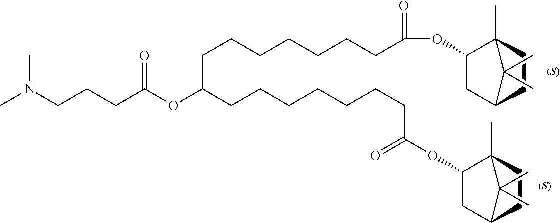 |
| C65 | 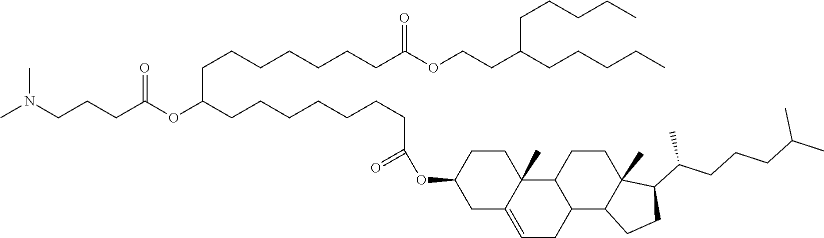 |
| C66 | 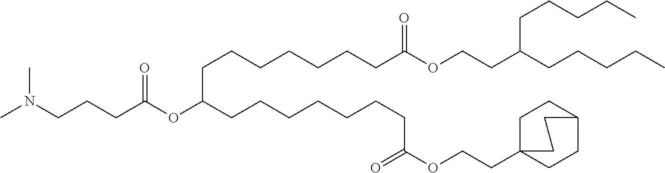 [SMILES: CCCCCC(CCCCC)CCOC(=O)CCCCCCCC(CCCCCCCC(=O)OCCC12CCC(CC1)CC2)OC(=O)CCCN(C)C] |
| C67 | 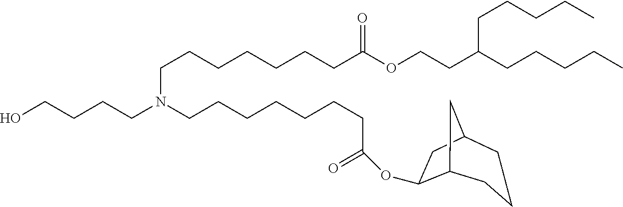 [SMILES: CCCCCC(CCCCC)CCOC(=O)CCCCCCCN(CCCCO)CCCCCCCC(=O)OC1CC2CCCC1C2] |
| C68 | 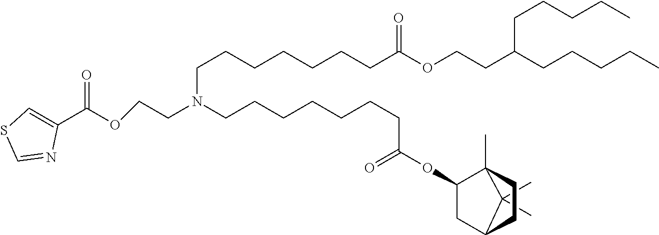 |
| C69 | 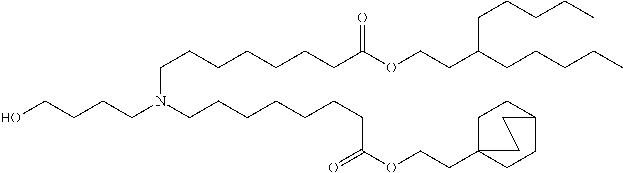 [SMILES: CCCCCC(CCCCC)CCOC(=O)CCCCCCCN(CCCCO)CCCCCCCC(=O)OCCC12CCC(CC1)CC2] |
| C70 | 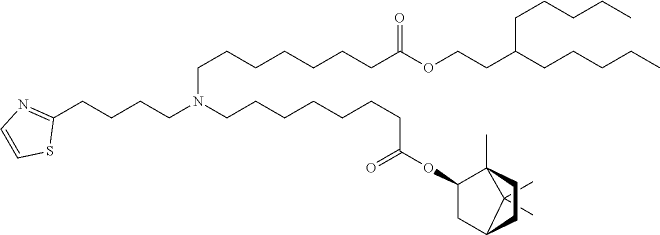 |
| C71 | 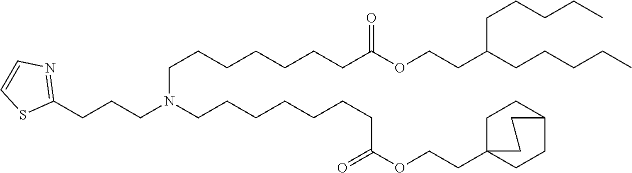 [SMILES: CCCCCC(CCCCC)CCOC(=O)CCCCCCCN(CCCCCCCC(=O)OCCC12CCC(CC1)CC2)CCCc1nccs1] |
| C72 | 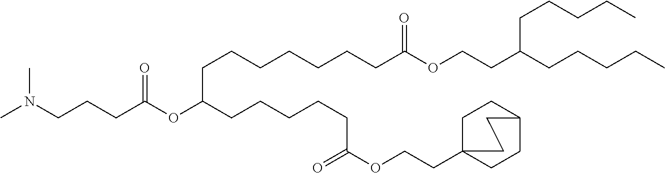 [SMILES: CCCCCC(CCCCC)CCOC(=O)CCCCCCCC(CCCCCC(=O)OCCC12CCC(CC1)CC2)OC(=O)CCCN(C)C] |
| C73 | 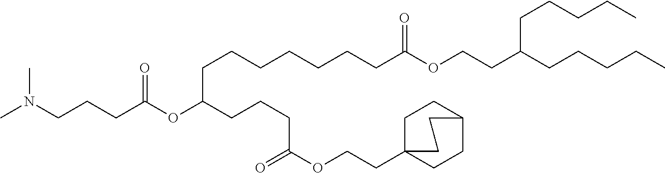 [SMILES: CCCCCC(CCCCC)CCOC(=O)CCCCCCCC(CCCC(=O)OCCC12CCC(CC1)CC2)OC(=O)CCCN(C)C] |
| C74 | 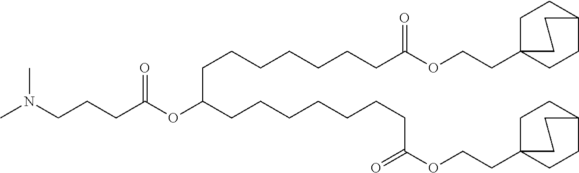 [SMILES: CN(C)CCCC(=O)OC(CCCCCCCC(=O)OCCC12CCC(CC1)CC2)CCCCCCCC(=O)OCCC12CCC(CC1)CC2] |
| C75 | 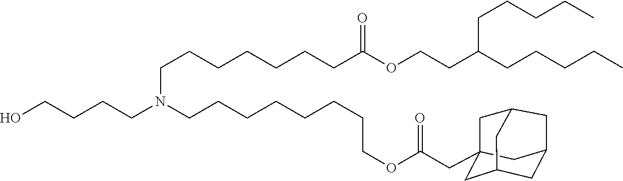 [SMILES: CCCCCC(CCCCC)CCOC(=O)CCCCCCCN(CCCCO)CCCCCCCCOC(=O)CC12CC3CC(CC(C3)C1)C2] |
| C76 | 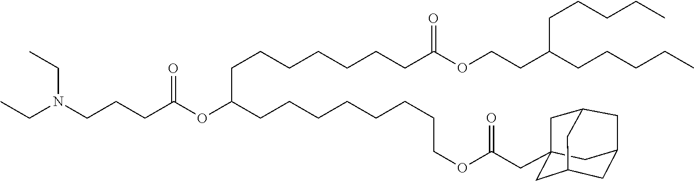 [SMILES: CCCCCC(CCCCC)CCOC(=O)CCCCCCCC(CCCCCCCCOC(=O)CC12CC3CC(CC(C3)C1)C2)OC(=O)CCCN(CC)CC] |
| C77 | 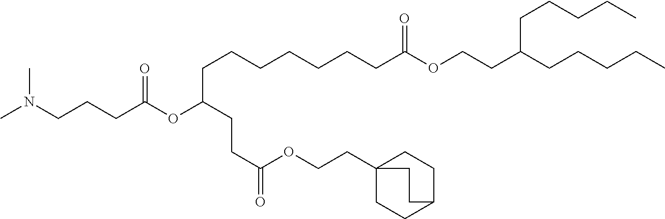 [SMILES: CCCCCC(CCCCC)CCOC(=O)CCCCCCCC(CCC(=O)OCCC12CCC(CC1)CC2)OC(=O)CCCN(C)C] |
| C78 | 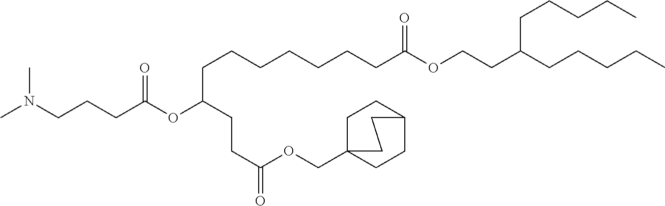 [SMILES: CCCCCC(CCCCC)CCOC(=O)CCCCCCCC(CCC(=O)OCC12CCC(CC1)CC2)OC(=O)CCCN(C)C] |
| C79 | 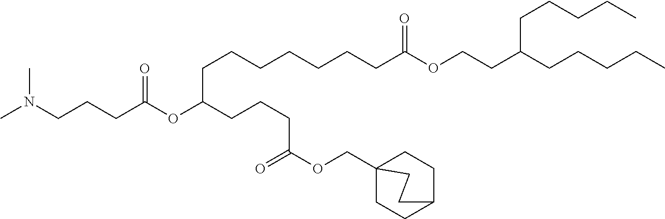 [SMILES: CCCCCC(CCCCC)CCOC(=O)CCCCCCCC(CCCC(=O)OCC12CCC(CC1)CC2)OC(=O)CCCN(C)C] |
| C80 | 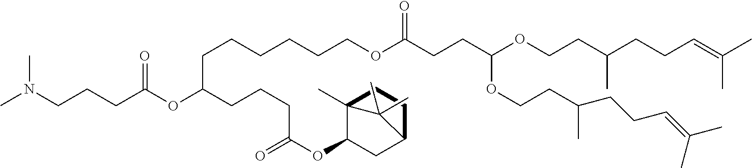 |
| C81 | 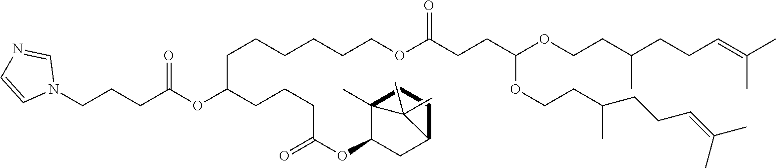 |

In some embodiments, an LNP of the present disclosure comprises an ionizable lipid disclosed in PCT Application PCT/US2023/065477, which is incorporated by reference herein, in its entirety.

In some embodiments, lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

In some embodiments, a compound of the present disclosure is represented by Formula (CX-I):

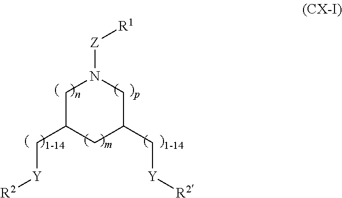

or a pharmaceutically acceptable salt thereof,
wherein

Z is selected from the group consisting of a bond,

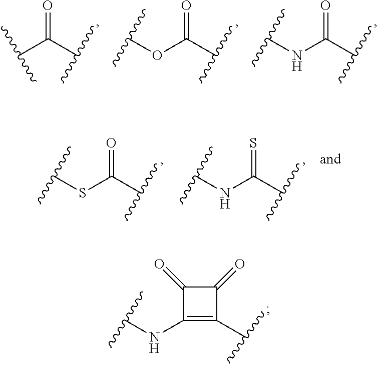

each Y is independently selected from the group consisting of

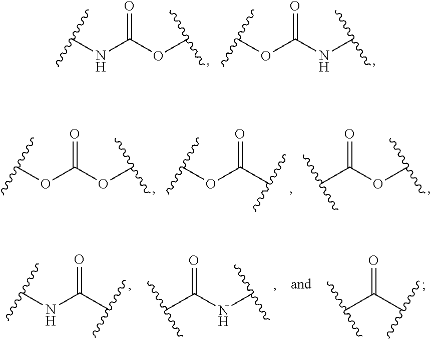

R¹ is —(CH₂)₁₋₆N(R^{a})₂ or —(CH₂)₁₋₆OH;
R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
R²′ is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—; each R^{a} is independently optionally substituted C₁-C₆ alkyl; or
two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by Formula (CX-i):

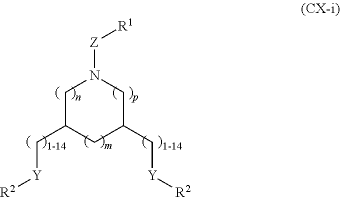

or a pharmaceutically acceptable salt thereof,
wherein
Z is selected from the group consisting of a bond,

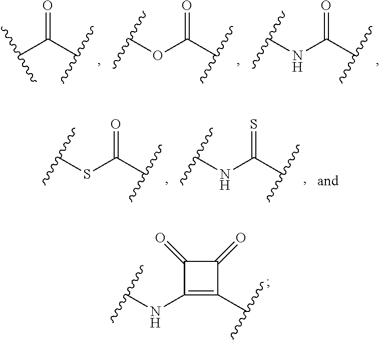

each Y is independently selected from the group consisting of

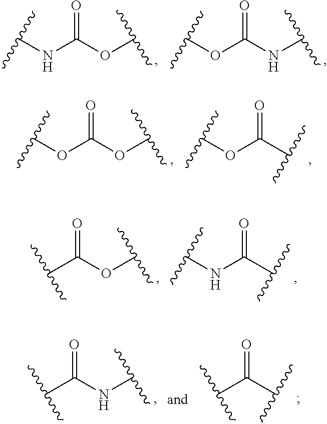

R¹ is —(CH₂)₁₋₆N(R^{a})₂;
R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by Formula (CX-I′), (CX-I″), (CX-I′″),

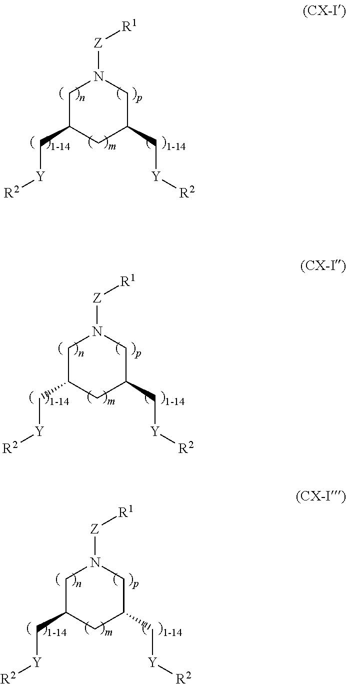

or a pharmaceutically acceptable salt thereof.

In some embodiments, a compound of the present disclosure is represented by Formula (CX-I-a), (CX-I-b), (CX-I-c), or (CX-I-d):

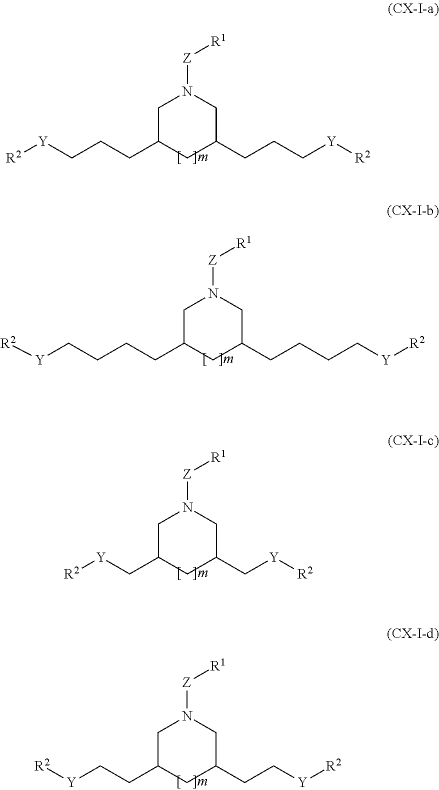

or a pharmaceutically acceptable salt thereof.

In some embodiments, a compound of the present disclosure is represented by Formula (CX-I-a′), (CX-I-b′), (CX-I-c′), or (CX-I-d′):

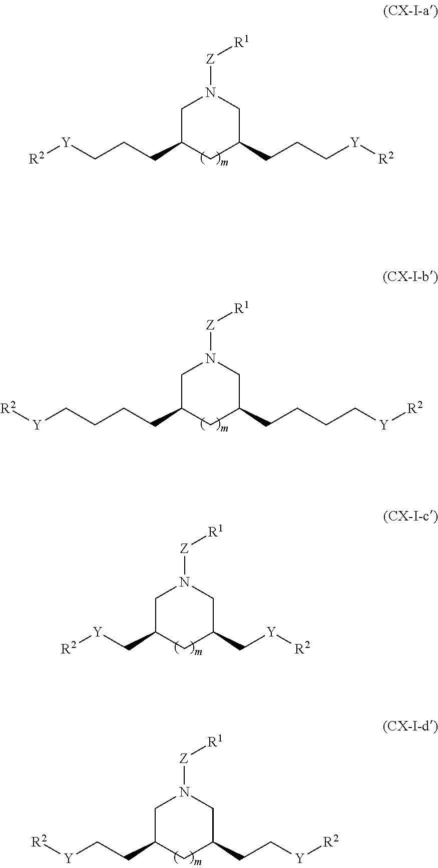

or a pharmaceutically acceptable salt thereof.

In some embodiments, a compound of the present disclosure is represented by Formula (CX-I-a″), (CX-I-b″), (CX-I-c″), or (CX-I-d″):

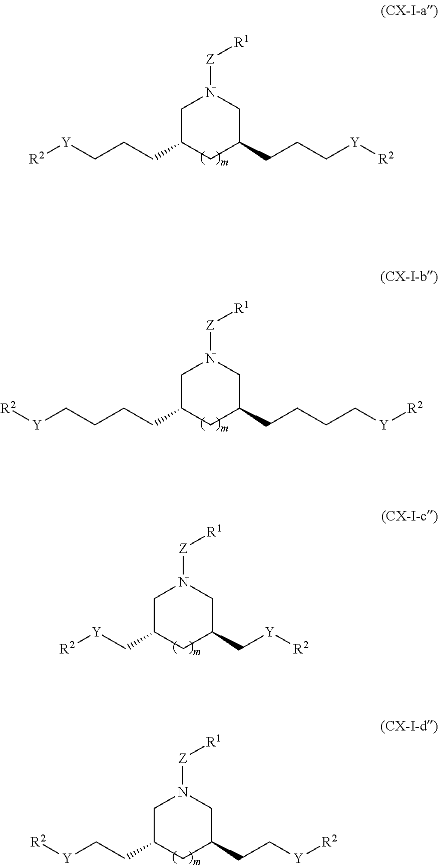

or a pharmaceutically acceptable salt thereof.

In some embodiments, a compound of the present disclosure is represented by Formula (CX-I-a″″), (CX-I-b″″), (CX-I-c′″), or (CX-I-d″″):

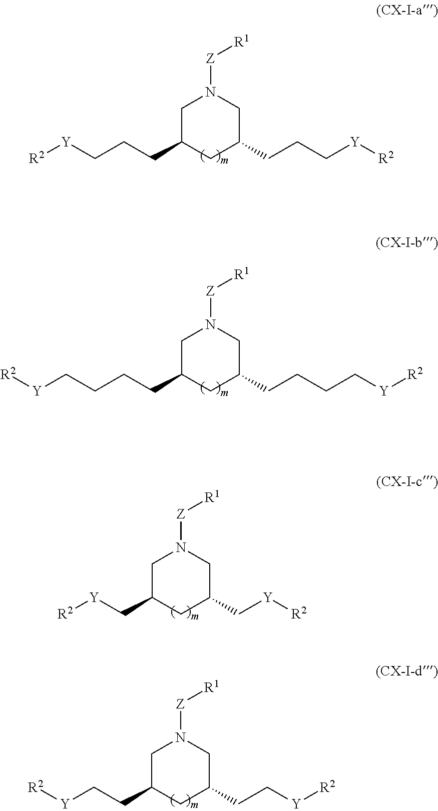

or a pharmaceutically acceptable salt thereof.

In some embodiments, a compound of the present disclosure is represented by Formula (CX-I-e) or (CX-I-f):

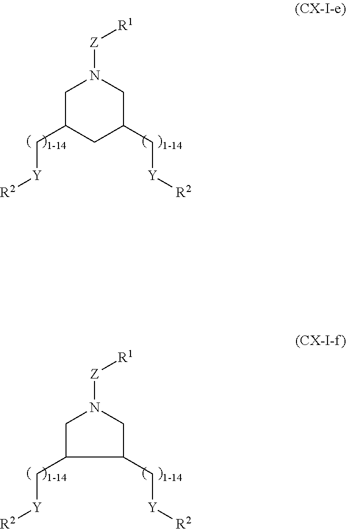

or a pharmaceutically acceptable salt thereof.

In some embodiments, a compound of the present disclosure is represented by Formula (CX-I-e′) or (CX-I-f′):

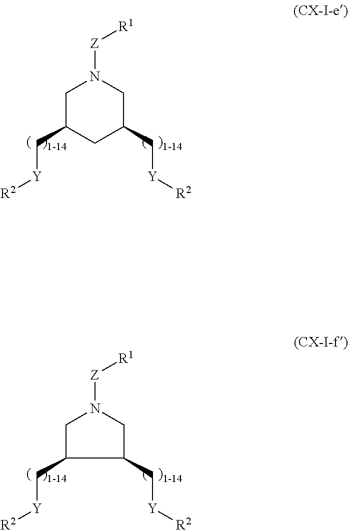

or a pharmaceutically acceptable salt thereof.

In some embodiments, a compound of the present disclosure is represented by Formula (CX-I-e″) or (CX-I-f″):

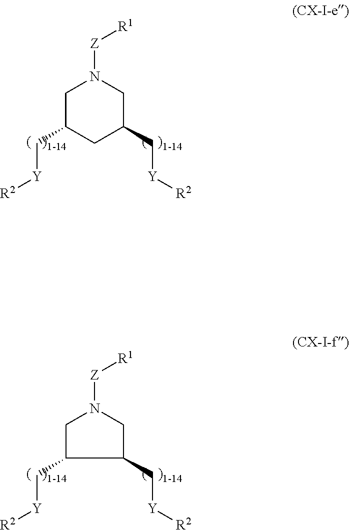

or a pharmaceutically acceptable salt thereof.

In some embodiments, a compound of the present disclosure is represented by Formula (CX-I-e′″) or (CX-I-f″):

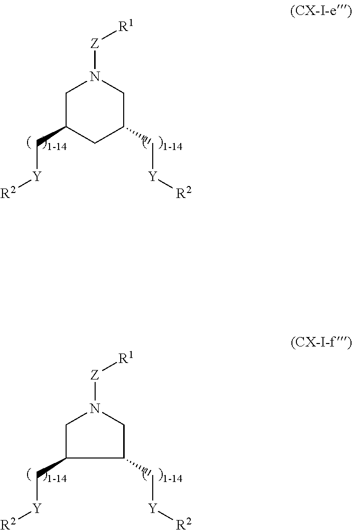

or a pharmaceutically acceptable salt thereof.

In some embodiments, a compound of the present disclosure is represented by Formula (CX-II):

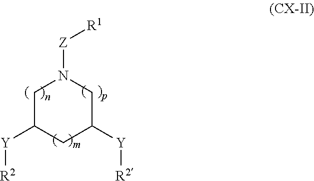

or a pharmaceutically acceptable salt thereof,
wherein
Z is selected from the group consisting of a bond,

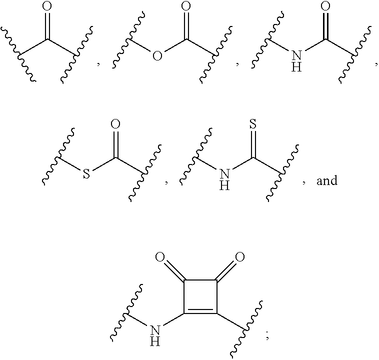

each Y is independently selected from the group consisting of

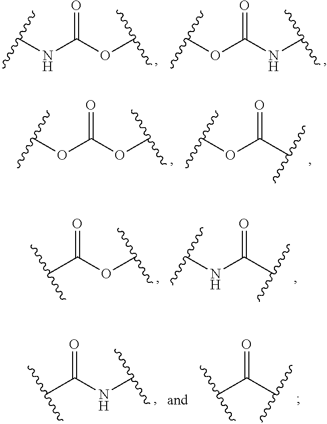

R¹ is —(CH₂)₁₋₆N(R^{a})₂ or —(CH₂)₁₋₆OH;
R² is optionally substituted C₅-C₃₆ alkyl or optionally substituted C₅-C₃₆ alkenyl, wherein 2 methylene units of R² are replaced with —O— to form an acetal within R², and wherein 1-6 methylene units of R² are optionally replaced with a group each independently selected from cyclopropylene, —O—, —OC(O)—, and —C(O)O—;
R²′ is optionally substituted C₁-C₃₆ alkyl or optionally substituted C₅-C₃₆ alkenyl, wherein 1-6 methylene units of R²′ are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by Formula (CX-ii):

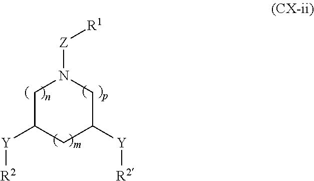

or a pharmaceutically acceptable salt thereof,
wherein
Z is selected from the group consisting of a bond,

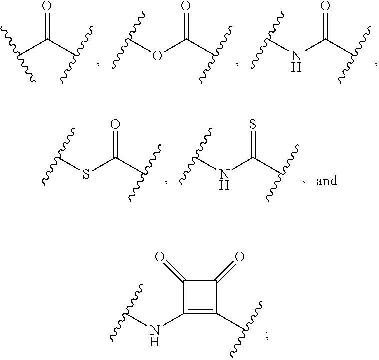

each Y is independently selected from the group consisting of

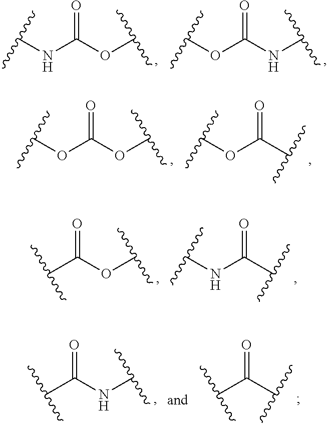

R¹ is —(CH₂)₁₋₆N(R^{a})₂;
R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
R²′ is optionally substituted C₁-C₃₆ alkyl, wherein 1-6 methylene units of R²′ are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by Formula (CX-II′), (CX-II″), (CX-II′″),

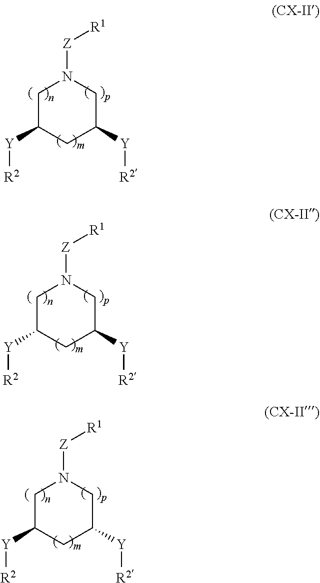

or a pharmaceutically acceptable salt thereof.

In some embodiments, a compound of the present disclosure is represented by Formula (CX-II-a)

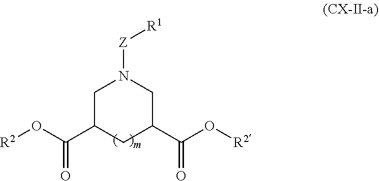

or a pharmaceutically acceptable salt thereof.

In some embodiments, a compound of the present disclosure is represented by Formula (CX-II-a′), (CX-II-a″), or (CX-II-a′″),

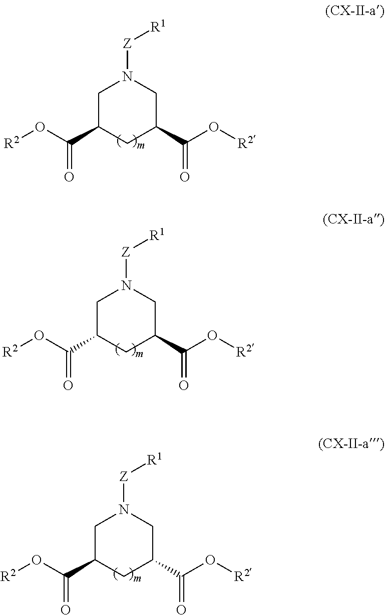

or a pharmaceutically acceptable salt thereof.

In some embodiments, a compound of the present disclosure is represented by Formula (CX-II-b), (CX-II-c), or (CX-II-d)

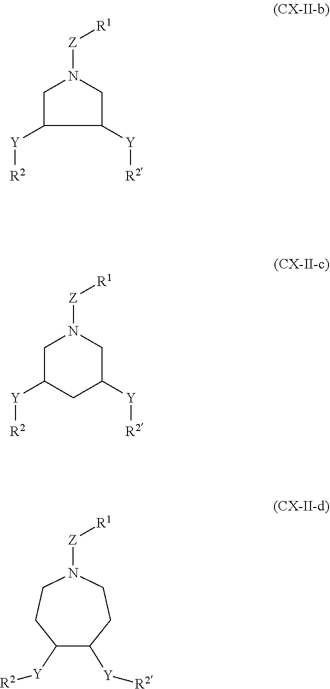

or a pharmaceutically acceptable salt thereof.

In some embodiments, a compound of the present disclosure is represented by Formula (CX-II-b′), (CX-II-c′), or (CX-II-d′)

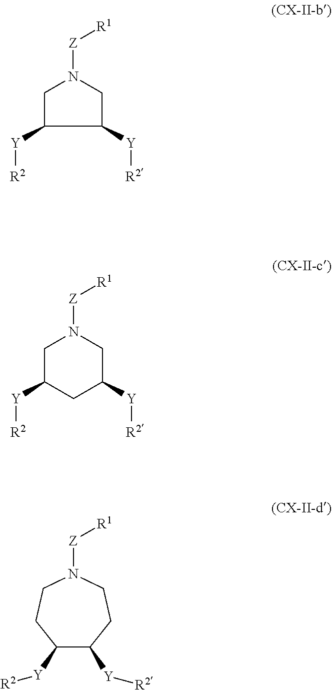

or a pharmaceutically acceptable salt thereof.

In some embodiments, a compound of the present disclosure is represented by Formula (CX-II-b″), (CX-II-c″), or (CX-II-d″)

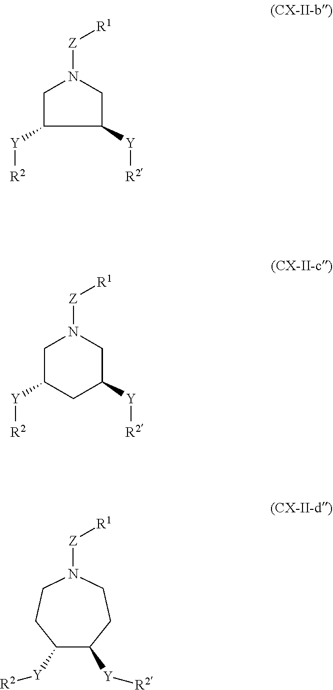

or a pharmaceutically acceptable salt thereof.

In some embodiments, a compound of the present disclosure is represented by Formula (CX-II-b′″), (CX-II-c′″), or (CX-II-d′″)

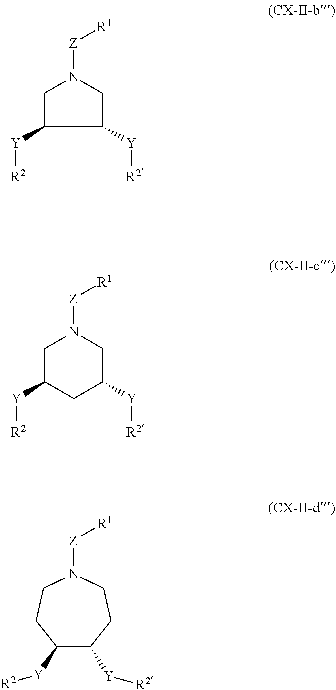

or a pharmaceutically acceptable salt thereof.

In some embodiments, a compound of the present disclosure is represented by is represented by formula (CX-II-e):

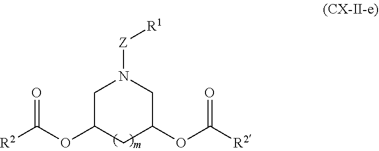

or a pharmaceutically acceptable salt thereof.

In some embodiments, a compound of the present disclosure is represented by Formula (CX-III)

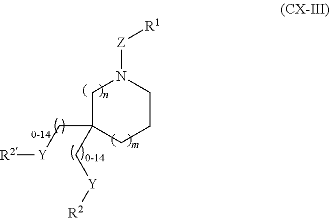

or a pharmaceutically acceptable salt thereof,
wherein
Z is selected from the group consisting of a bond,

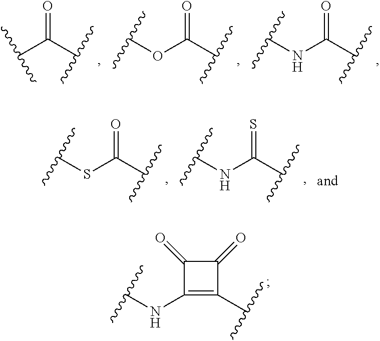

each Y is independently selected from the group consisting of

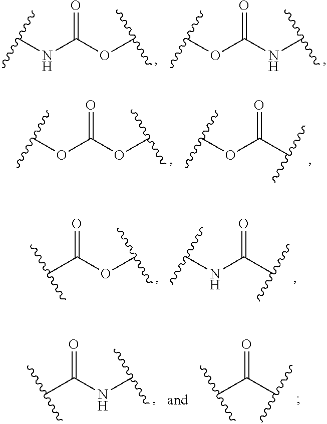

R¹ is —(CH₂)₁₋₆N(R^{a})₂ or —(CH₂)₁₋₆OH;
R² is optionally substituted C₅-C₃₆ alkyl or optionally substituted C₅-C₃₆ alkenyl, wherein 2 methylene units of R² are replaced with —O- to form an acetal within R², and wherein 1-6 methylene units of R² are optionally replaced with a group each independently selected from cyclopropylene, —O—, —OC(O)—, and —C(O)O—;
R²′ is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2; and
n is 1 or 2.

In some embodiments, a compound of the present disclosure is represented by Formula (CX-iii)

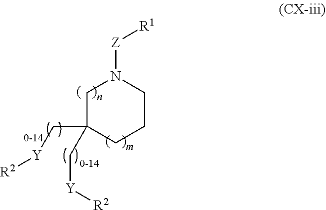

or a pharmaceutically acceptable salt thereof,
wherein
Z is selected from the group consisting of a bond,

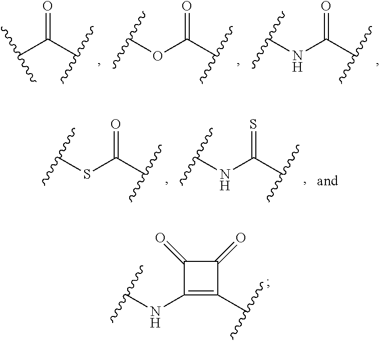

each Y is independently selected from the group consisting of

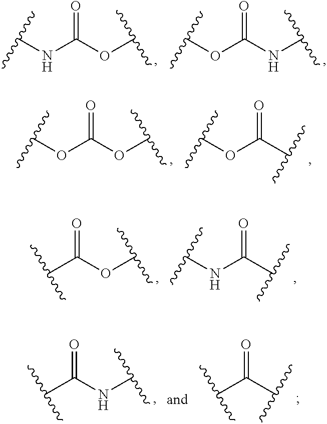

R¹ is —(CH₂)₁₋₆N(R^{a})₂;
each R² is independently optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2; and
n is 1 or 2.

In some embodiments, a compound of the present disclosure is represented by Formula (CX-III-a), (CX-III-b), or (CX-III-c):

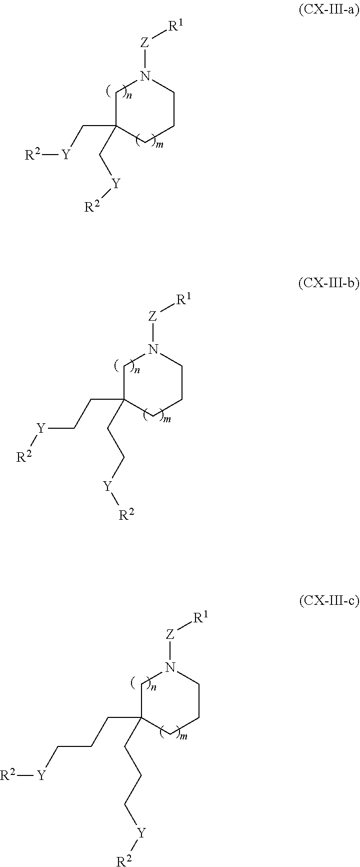

or a pharmaceutically acceptable salt thereof.

In some embodiments, a compound of the present disclosure is represented by Formula (CX-III-d) or (CX-III-e)

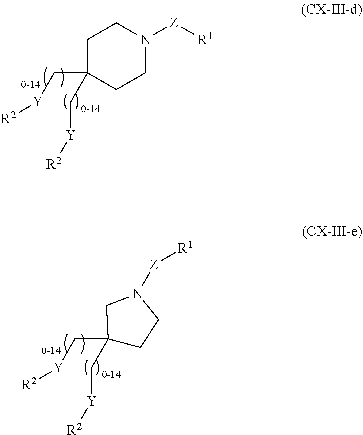

or a pharmaceutically acceptable salt thereof.

In some embodiments, a compound of the present disclosure is represented by Formula (CX-IV)

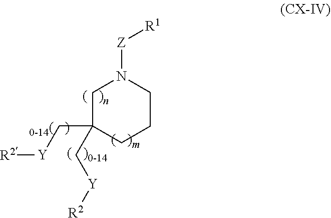

or a pharmaceutically acceptable salt thereof,
wherein
Z is selected from the group consisting of a bond,

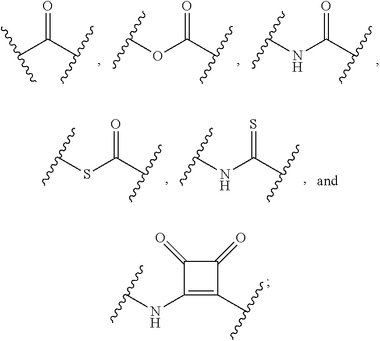

each Y is independently selected from the group consisting of

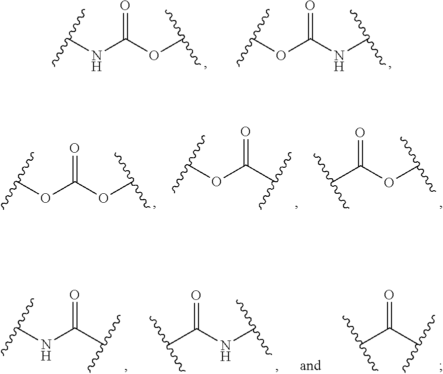

R¹ is —(CH₂)₁₋₆N(R^{a})₂ or —(CH₂)₁₋₆OH; R² is C₃-C₃₆ branched alkyl or optionally substituted C₃-C₃₆ branched alkenyl, wherein 1-6 methylene units of R² are optionally replaced with a group each independently selected from cyclopropylene and —O—;
R²′ is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2; and
n is 1 or 2.

In some embodiments, a compound is represented by formula (CX-IV-a), (CX-IV-b), or (CX-IV-c):

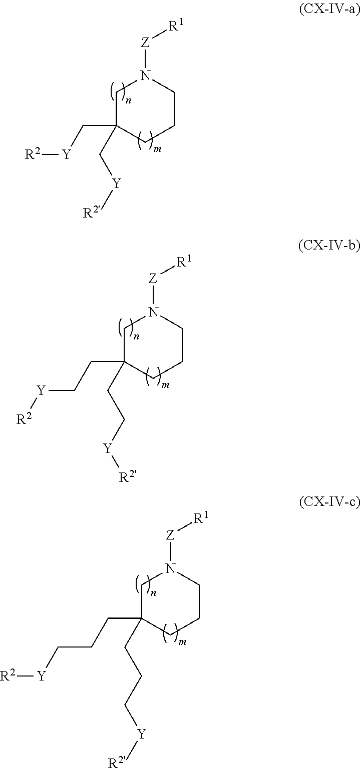

or a pharmaceutically acceptable salt thereof.

In some embodiments, a compound of the present disclosure compound is represented by formula (CX-IV-d) or (CX-IV-e):

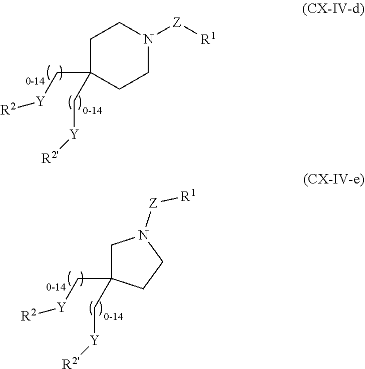

or a pharmaceutically acceptable salt thereof.

### Z

In some embodiments, Z is selected from the group consisting of a bond,

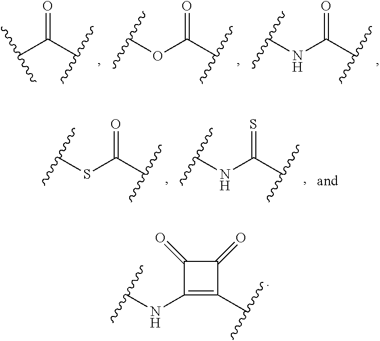

In some embodiments, Z is selected from the group consisting of

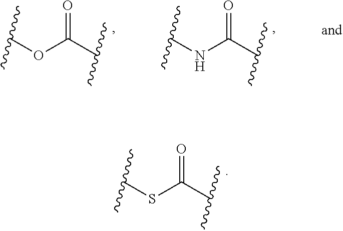 [SMILES: *OC(*)=O ; *NC(*)=O ; *SC(*)=O]

In some embodiments, Z is

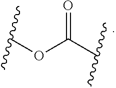 [SMILES: *OC(*)=O]

In some embodiments, Z is selected from the group consisting of a bond,

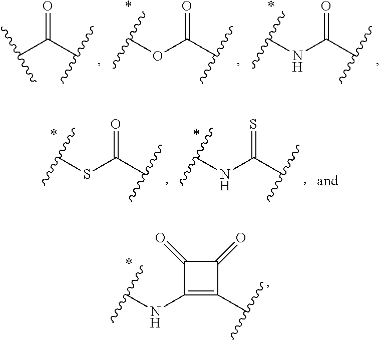

wherein R¹ is attached at the position denoted by *

In some embodiments, Z is selected from the group consisting of

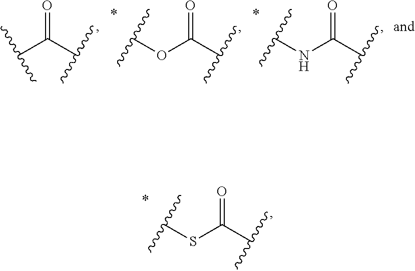

wherein R¹ is attached at the position denoted by*

In some embodiments, Z is

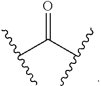 [SMILES: *C(*)=O]

In some embodiments, Z is

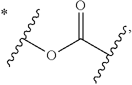

wherein R¹ is attached at the position denoted by *. In some embodiments, Z is

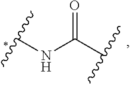

wherein R¹ is attached at the position denoted by *. In some embodiments, Z is

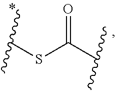

wherein R¹ is attached at the position denoted by *.

### Y

In some embodiments, each Y is independently selected from the group consisting of

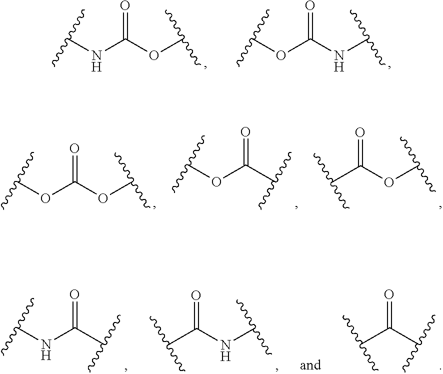

In some embodiments, Y is selected from the group consisting of

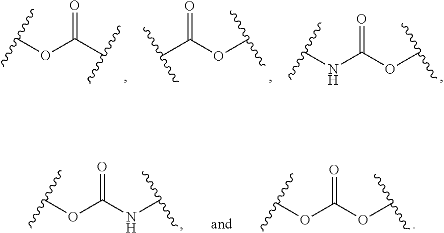 [SMILES: *OC(*)=O ; *OC(*)=O ; *NC(=O)O* ; *NC(=O)O* ; *OC(=O)O*]

In some embodiments, Y is selected from the group consisting of

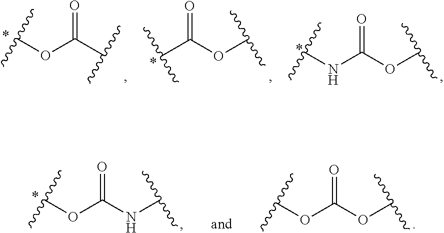

wherein R² is attached at the position denoted by *. In some embodiments, Y is

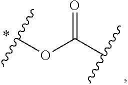

wherein R² is attached at the position denoted by * In some embodiments, Y is

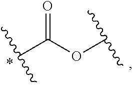

wherein R² is attached at the position denoted by * In some embodiments, Y is

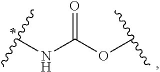

wherein R² is attached at the position denoted by * In some embodiments, Y is

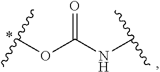

wherein R² is attached at the position denoted by *. In some embodiments, Y is

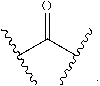 [SMILES: *C(*)=O]

In some embodiments, Y is

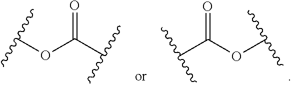 [SMILES: *OC(*)=O ; *OC(*)=O]

In some embodiments, Y is

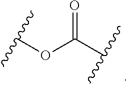 [SMILES: *OC(*)=O]

In some embodiments, Y is

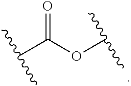 [SMILES: *OC(*)=O]

### R¹

In some embodiments, R¹ is —(CH₂)₁₋₆N(R^{a})₂ or —(CH₂)₁₋₆OH. In some embodiments, R¹ is —(CH₂)₁₋₆OH. In some embodiments, R¹ is —(CH₂)₁₋₆N(R^{a})₂. In some embodiments, R¹ is —(CH₂)₂N(R^{a})₂. In some embodiments, R¹ is —(CH₂)₃N(R^{a})₂. In some embodiments, R¹ is —(CH₂)₄N(R^{a})₂. In some embodiments, R¹ is —(CH₂)₁₋₆N(Me)₂. In some embodiments, R¹ is —(CH₂)₁₋₆N(Et)₂. In some embodiments, R¹ is —(CH₂)₁₋₆N(n-Pr)₂. In some embodiments, R¹ is (CH₂)₁₋₆N(i-Pr)₂. In some embodiments, R¹ is —(CH₂)₂N(Me)₂. In some embodiments, R¹ is (CH₂)₃N(Me)₂. In some embodiments, R¹ is —(CH₂)₄N(Me)₂. In some embodiments, R¹ is —(CH₂)₂N(Et)₂. In some embodiments, R¹ is —(CH₂)₃N(Et)₂. In some embodiments, R¹ is —(CH₂)₄N(Et)₂.

In some embodiments, R¹ is selected from the group consisting of

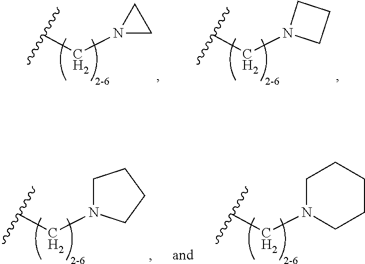

In some embodiments, R¹ is selected from the group consisting of

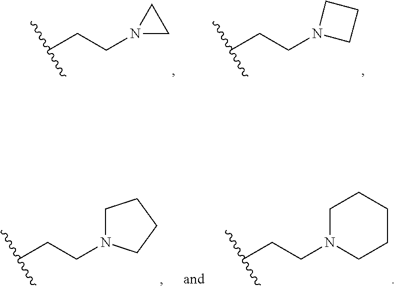 [SMILES: *CCN1CC1 ; *CCN1CCC1 ; *CCN1CCCC1 ; *CCN1CCCCC1]

In some embodiments, R¹ is selected from the group consisting of

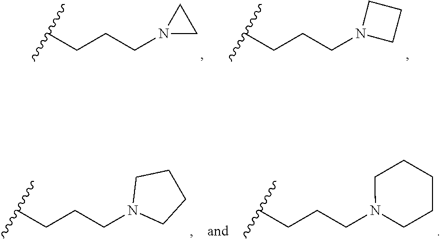 [SMILES: *CCCN1CC1 ; *CCCN1CCC1 ; *CCCN1CCCC1 ; *CCCN1CCCCC1]

R² and R²′

In some embodiments, R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—. In some embodiments, R² is optionally substituted C₁-C₃₂ alkyl or optionally substituted C₂-C₃₂ alkenyl, wherein 1-6 methylene units of R² are optionally replaced with a group each independently selected from cyclopropylene, —O—, —OC(O)—, and —C(O)O—. In some embodiments, R² is optionally substituted C₁-C₃₀ alkyl or optionally substituted C₂-C₃₀ alkenyl, wherein 1-6 methylene units of R² are optionally replaced with a group each independently selected from cyclopropylene, —O—, —OC(O)—, and —C(O)O—. In some embodiments, R² is optionally substituted C₁-C₂₄ alkyl or optionally substituted C₂-C₂₄ alkenyl, wherein 1-6 methylene units of R² are optionally replaced with a group each independently selected from cyclopropylene, —O—, —OC(O)—, and —C(O)O—. In some embodiments, R² is optionally substituted C₁-C₂₄ alkyl or optionally substituted C₂-C₂₄ alkenyl, wherein 1-6 methylene units of R² are replaced with a group each independently selected from cyclopropylene, —O—, —OC(O)—, and —C(O)O—. In some embodiments, R² is optionally substituted C₁-C₂₄ alkyl or optionally substituted C₂-C₂₄ alkenyl. In some embodiments, R² is optionally substituted C₁₀-C₂₄ alkyl or optionally substituted C₁₀-C₂₄ alkenyl, wherein 1-6 methylene units of R² are replaced with —O—.

In some embodiments, R² is optionally substituted C₅-C₃₆ alkyl or optionally substituted C₅-C₃₆ alkenyl, wherein 2 methylene units of R² are replaced with —O- to form an acetal within R², and wherein 1-6 methylene units of R² are optionally replaced with a group each independently selected from cyclopropylene, —O—, —OC(O)—, and —C(O)O—; and R²′ is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—.

In some embodiments, R² is optionally substituted C₁₀-C₂₄ alkyl or optionally substituted C₁₀-C₂₄ alkenyl, wherein 2 methylene units of R² are replaced with —O- to form an acetal within R² and wherein 1-6 methylene units of R² are optionally replaced with a group each independently selected from cyclopropylene, —O—, —OC(O)—, and —C(O)O—; and R²′ is optionally substituted C₁₀-C₃₆ branched alkyl or optionally substituted C₁₀-C₃₆ alkenyl, wherein 1-6 methylene units of R² are optionally replaced with a group each independently selected from cyclopropylene, —O—, —OC(O)—, and —C(O)O—.

In some embodiments, R² is C₃-C₃₆ branched alkyl or optionally substituted C₃-C₃₆ branched alkenyl, wherein 1-6 methylene units of R² are optionally replaced with a group each independently selected from cyclopropylene and —O—; and R²′ is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—. In some embodiments, R² is optionally substituted C₁₀-C₂₄ branched alkyl or optionally substituted C₁₀-C₂₄ branched alkenyl, wherein 1-3 methylene units of R² are optionally replaced with —O—; and R²′ is optionally substituted C₁₀-C₃₆ alkyl or optionally substituted C₁₀-C₃₆ alkenyl, wherein 1-6 methylene units of R² are optionally replaced with a group each independently selected from cyclopropylene, —O—, —OC(O)—, and —C(O)O—.

In some embodiments, R² and/or R²′ is

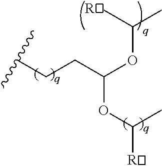

wherein each q is independently selected from 0-12 and each R° is independently selected, and is as described and defined herein.

In some embodiments, R² and/or R²′ is

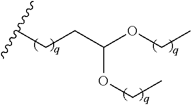

wherein each q is independently selected from 0-12.

In some embodiments, R² is optionally substituted C₅-C₃₆ alkyl or optionally substituted C₅-C₃₆ alkenyl, wherein 2 methylene units of R² are replaced with —O- to form an acetal within R², and wherein 1-6 methylene units of R² are optionally replaced with a group each independently selected from cyclopropylene, —O—, —OC(O)—, and —C(O)O—; R²′ is optionally substituted C₁-C₃₆ alkyl or optionally substituted C₅-C₃₆ alkenyl, wherein 1-6 methylene units of R²′ are optionally replaced with a group each independently selected from cyclopropylene, —O—, —OC(O)—, and —C(O)O—.

In some embodiments, R² is optionally substituted C₁₀-C₂₄ alkyl, wherein 2 methylene units of R² are replaced with —O- to form an acetal within R²; and R²′ is optionally substituted C₁₀-C₂₄ alkyl, wherein 2 methylene units of R²′ are replaced with —O- to form an acetal within R²′.

In some embodiments, each q is independently selected from 0-6. In some embodiments, each q is independently selected from 0-8. In some embodiments, each q is independently selected from 0-10. In some embodiments, each q is independently selected from 0-12.

In some embodiments, R² is optionally substituted C₁₀-C₂₄ alkyl or optionally substituted C₁₀-C₂₄ alkenyl, wherein 2 methylene units of R² are replaced with —O- to form an acetal within R² and wherein 1-6 methylene units of R² are optionally replaced with a group each independently selected from cyclopropylene, —O—, —OC(O)—, and —C(O)O—; and R²′ is optionally substituted C₁₀-C₂₄ alkenyl, wherein 1-3 methylene units of R²′ are optionally replaced with —O—.

In some embodiments, R² is selected from the group consisting of

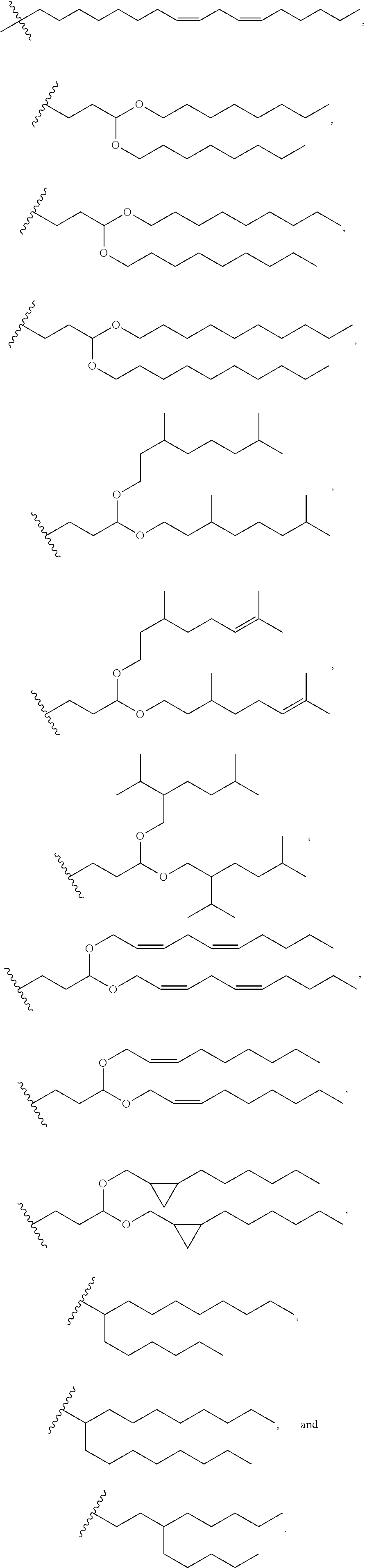

In some embodiments, R² is

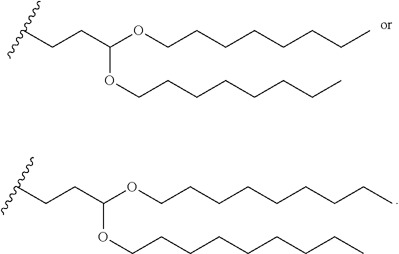 [SMILES: *CCC(OCCCCCCCC)OCCCCCCCC ; *CCC(OCCCCCCCCC)OCCCCCCCCC]

In some embodiments, R² is

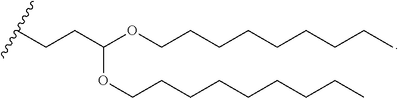 [SMILES: *CCC(OCCCCCCCCC)OCCCCCCCCC]

In some embodiments R² is

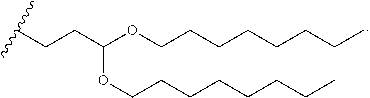 [SMILES: *CCC(OCCCCCCCC)OCCCCCCCC]

In some embodiments, R² is selected from the group consisting of

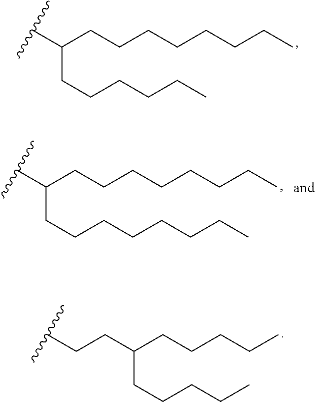 [SMILES: *C(CCCCCC)CCCCCCCC ; *C(CCCCCCCC)CCCCCCCC ; *CCC(CCCCC)CCCCC]

In some embodiments, R² is optionally substituted C₁-C₃₆ alkyl, wherein 1-6 methylene units of R²′ are optionally replaced with a group each independently selected from cyclopropylene, —O—, —OC(O)—, and —C(O)O—. In some embodiments, R²′ is optionally substituted C₁-C₃₂ alkyl, wherein 1-6 methylene units of R²′ are optionally replaced with a group each independently selected from cyclopropylene, —O—, —OC(O)—, and —C(O)O—. In some embodiments, R²′ is optionally substituted C₁-C₃₀ alkyl, wherein 1-6 methylene units of R²′ are optionally replaced with a group each independently selected from cyclopropylene, —O—, —OC(O)—, and —C(O)O—. In some embodiments, R²′ is optionally substituted C₁-C₂₄ alkyl, wherein 1-6 methylene units of R²′ are optionally replaced with a group each independently selected from cyclopropylene, —O—, —OC(O)—, and —C(O)O—. In some embodiments, R²′ is optionally substituted C₁-C₂₄ alkyl, wherein 1-6 methylene units of R²′ are replaced with a group each independently selected from —O—, —OC(O)—, and —C(O)O—. In some embodiments, R²′ is optionally substituted C₁-C₂₄ alkyl. In some embodiments, R²′ is optionally substituted C₁₀-C₂₄ alkyl, wherein 1-6 methylene units of R²′ are replaced with —O—.

In some embodiments, R²′ is selected from the group consisting of

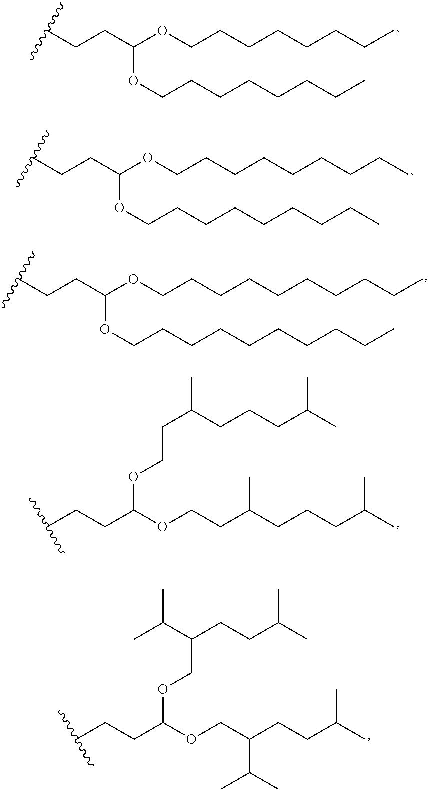

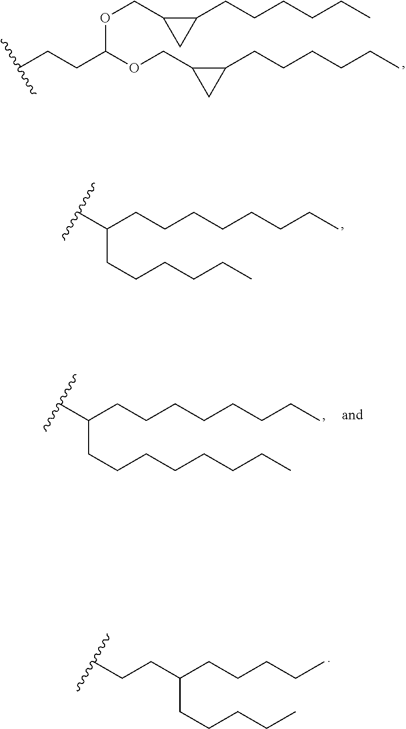 [SMILES: *CCC(OCC1CC1CCCCCC)OCC1CC1CCCCCC ; *C(CCCCCC)CCCCCCCC ; *C(CCCCCCCC)CCCCCCCC ; *CCC(CCCCC)CCCCC]

In some embodiments, R² and R²′ are each independently selected from the group consisting of

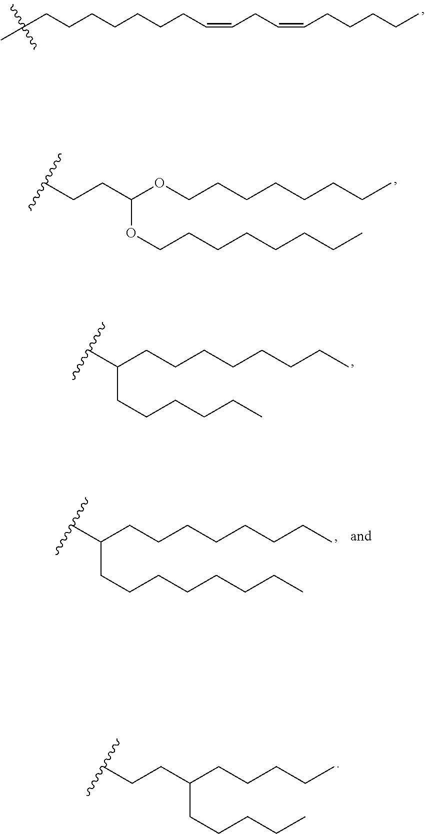 [SMILES: *CCCCCCC/C=C\C/C=C\CCCCC ; *CCC(OCCCCCCCC)OCCCCCCCC ; *C(CCCCCC)CCCCCCCC ; *C(CCCCCCCC)CCCCCCCC ; *CCC(CCCCC)CCCCC]

In some embodiments, R²′ is selected from the group consisting of

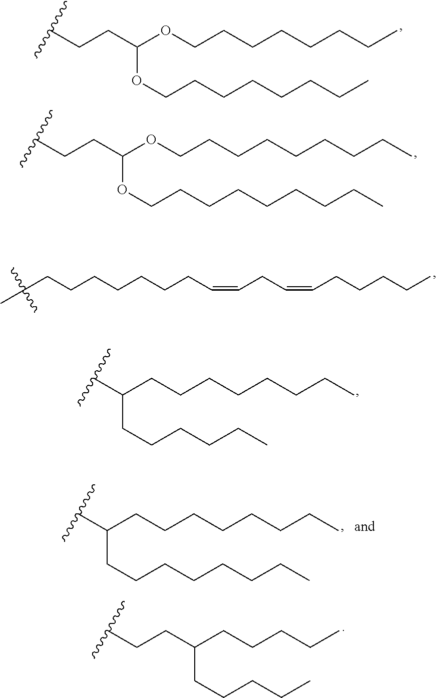

In some embodiments, R²′ is

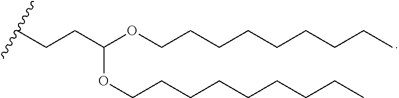 [SMILES: *CCC(OCCCCCCCCC)OCCCCCCCCC]

In some embodiments, R²′ is

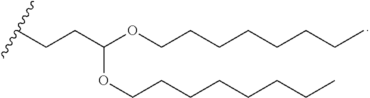 [SMILES: *CCC(OCCCCCCCC)OCCCCCCCC]

In some embodiments, R²′ is selected from the group consisting of

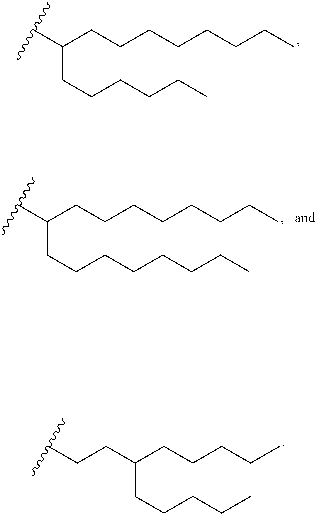 [SMILES: *C(CCCCCC)CCCCCCCC ; *C(CCCCCCCC)CCCCCCCC ; *CCC(CCCCC)CCCCC]

In some embodiments, R² is selected from the group consisting of

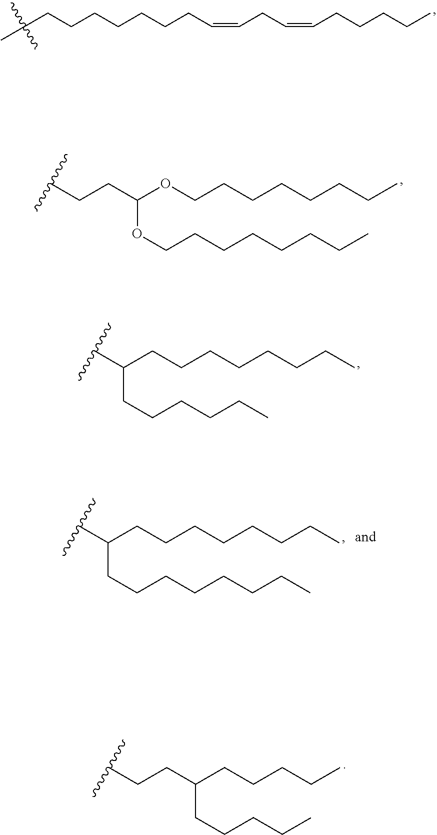 [SMILES: *CCCCCCC/C=C\C/C=C\CCCCC ; *CCC(OCCCCCCCC)OCCCCCCCC ; *C(CCCCCC)CCCCCCCC ; *C(CCCCCCCC)CCCCCCCC ; *CCC(CCCCC)CCCCC]

In some embodiments, R²′ is selected from the group consisting of

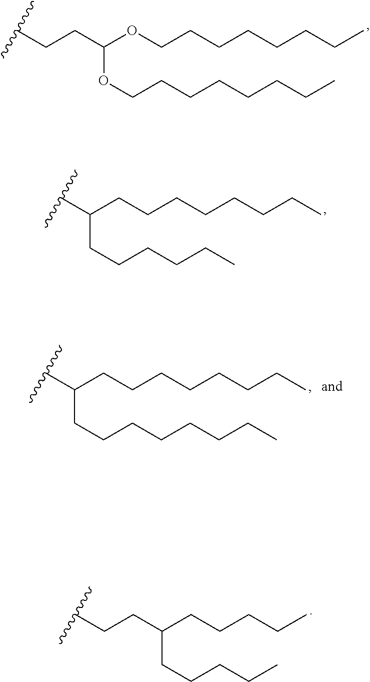 [SMILES: *CCC(OCCCCCCCC)OCCCCCCCC ; *C(CCCCCC)CCCCCCCC ; *C(CCCCCCCC)CCCCCCCC ; *CCC(CCCCC)CCCCC]

In some embodiments, the present disclosure includes a compound selected from any lipid in Table (IV) below or a pharmaceutically acceptable salt thereof:

TABLE IV
Non-Limiting Examples of Ionizable Lipids

| | Compound |
|---|---|
| Structure | No. |
| 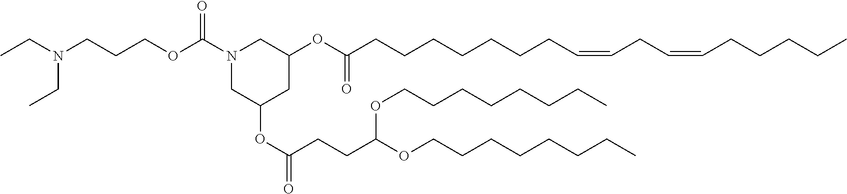 [SMILES: CCCCC/C=C\C/C=C\CCCCCCCC(=O)OC1CC(OC(=O)CCC(OCCCCCCCC)OCCCCCCCC)CN(C(=O)OCCCN(CC)CC)C1] | CX-1 |
| 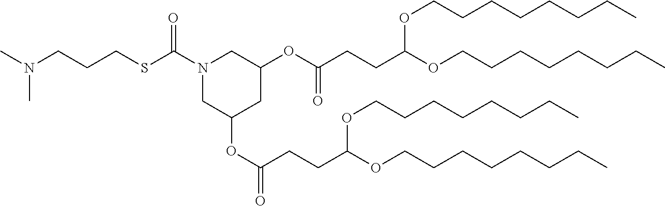 [SMILES: CCCCCCCCOC(CCC(=O)OC1CC(OC(=O)CCC(OCCCCCCCC)OCCCCCCCC)CN(C(=O)SCCCN(C)C)C1)OCCCCCCCC] | CX-2 |
| 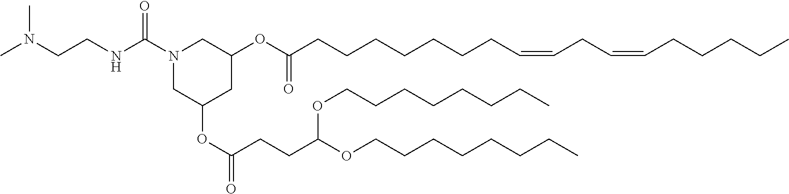 [SMILES: CCCCC/C=C\C/C=C\CCCCCCCC(=O)OC1CC(OC(=O)CCC(OCCCCCCCC)OCCCCCCCC)CN(C(=O)NCCN(C)C)C1] | CX-3 |
| 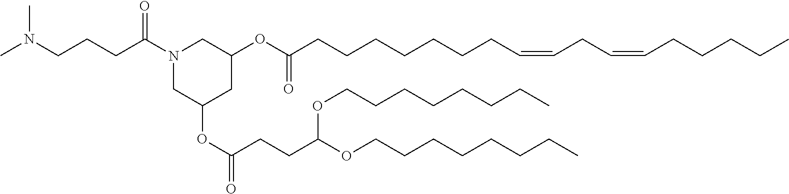 [SMILES: CCCCC/C=C\C/C=C\CCCCCCCC(=O)OC1CC(OC(=O)CCC(OCCCCCCCC)OCCCCCCCC)CN(C(=O)CCCN(C)C)C1] | CX-4 |
| 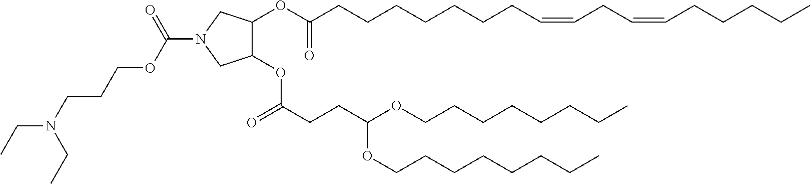 [SMILES: CCCCC/C=C\C/C=C\CCCCCCCC(=O)OC1CN(C(=O)OCCCN(CC)CC)CC1OC(=O)CCC(OCCCCCCCC)OCCCCCCCC] | CX-5 |
| 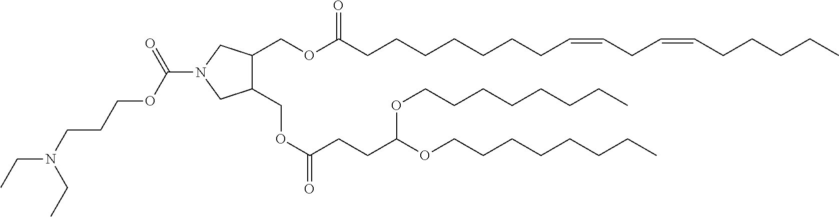 [SMILES: CCCCC/C=C\C/C=C\CCCCCCCC(=O)OCC1CN(C(=O)OCCCN(CC)CC)CC1COC(=O)CCC(OCCCCCCCC)OCCCCCCCC] | CX-6 |
| 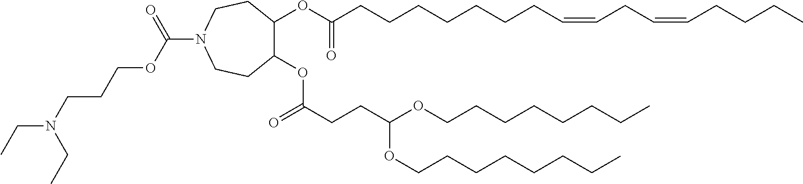 [SMILES: CCCC/C=C\C/C=C\CCCCCCCC(=O)OC1CCN(C(=O)OCCCN(CC)CC)CCC1OC(=O)CCC(OCCCCCCCC)OCCCCCCCC] | CX-7 |
| 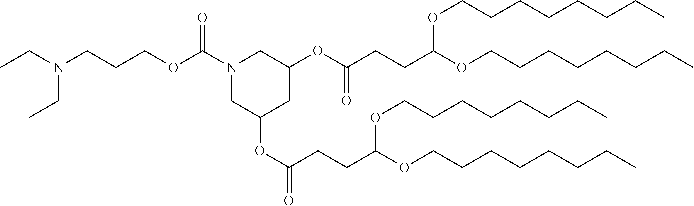 [SMILES: CCCCCCCCOC(CCC(=O)OC1CC(OC(=O)CCC(OCCCCCCCC)OCCCCCCCC)CN(C(=O)OCCCN(CC)CC)C1)OCCCCCCCC] | CX-8 |
| 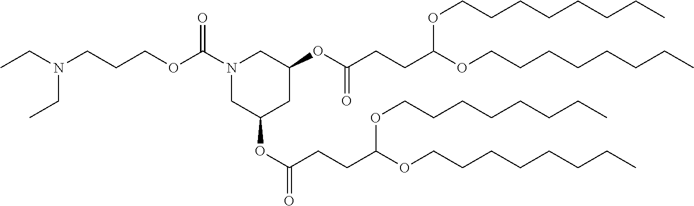 [SMILES: CCCCCCCCOC(CCC(=O)O[C@@H]1C[C@H](OC(=O)CCC(OCCCCCCCC)OCCCCCCCC)CN(C(=O)OCCCN(CC)CC)C1)OCCCCCCCC] | CX-8a |
| 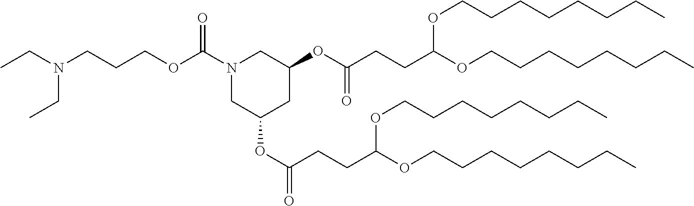 [SMILES: CCCCCCCCOC(CCC(=O)O[C@H]1C[C@H](OC(=O)CCC(OCCCCCCCC)OCCCCCCCC)CN(C(=O)OCCCN(CC)CC)C1)OCCCCCCCC] | CX-8b |
| 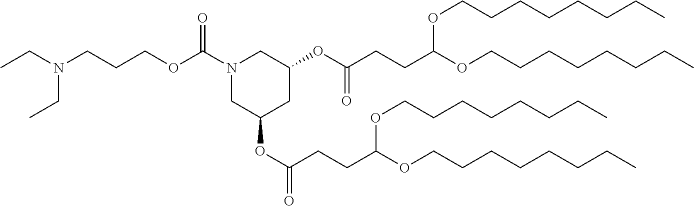 [SMILES: CCCCCCCCOC(CCC(=O)O[C@@H]1C[C@@H](OC(=O)CCC(OCCCCCCCC)OCCCCCCCC)CN(C(=O)OCCCN(CC)CC)C1)OCCCCCCCC] | CX-8c |
| 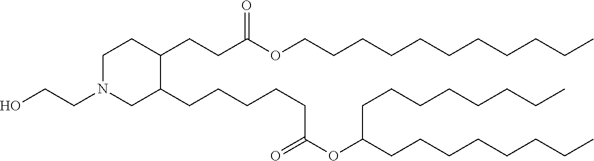 [SMILES: CCCCCCCCCCCOC(=O)CCC1CCN(CCO)CC1CCCCCC(=O)OC(CCCCCCCC)CCCCCCCC] | CX-9 |
| 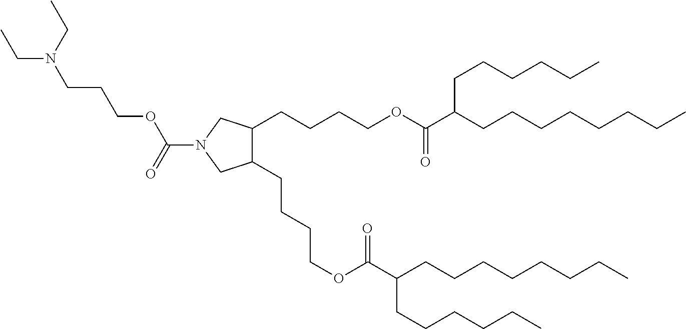 [SMILES: CCCCCCCCC(CCCCCC)C(=O)OCCCCC1CN(C(=O)OCCCN(CC)CC)CC1CCCCOC(=O)C(CCCCCC)CCCCCCCC] | CX-10 |
| 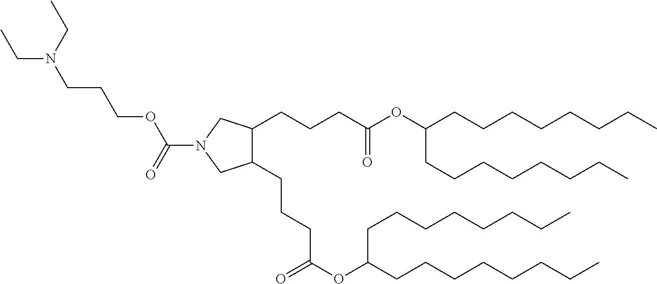 [SMILES: CCCCCCCCC(CCCCCCCC)OC(=O)CCCC1CN(C(=O)OCCCN(CC)CC)CC1CCCC(=O)OC(CCCCCCCC)CCCCCCCC] | CX-11 |
| 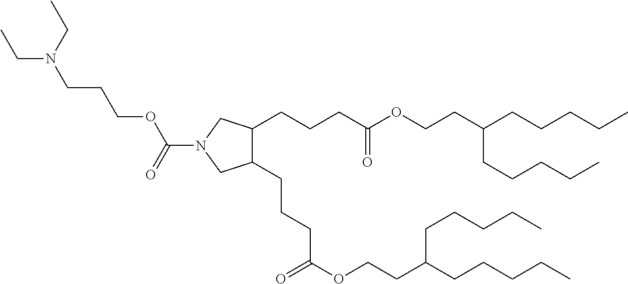 [SMILES: CCCCCC(CCCCC)CCOC(=O)CCCC1CN(C(=O)OCCCN(CC)CC)CC1CCCC(=O)OCCC(CCCCC)CCCCC] | CX-12 |
| 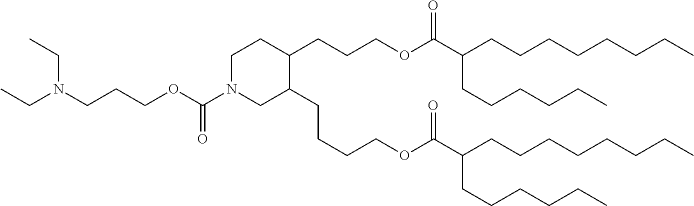 [SMILES: CCCCCCCCC(CCCCCC)C(=O)OCCCCC1CN(C(=O)OCCCN(CC)CC)CCC1CCCOC(=O)C(CCCCCC)CCCCCCCC] | CX-13 |
| 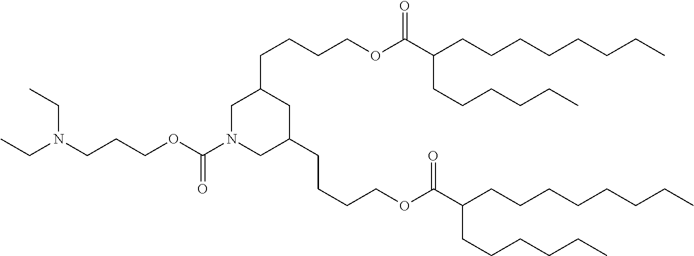 [SMILES: CCCCCCCCC(CCCCCC)C(=O)OCCCCC1CC(CCCCOC(=O)C(CCCCCC)CCCCCCCC)CN(C(=O)OCCCN(CC)CC)C1] | CX-14 |
| 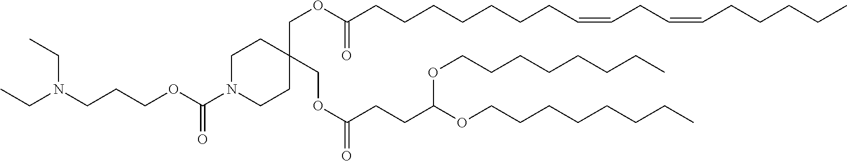 [SMILES: CCCCC/C=C\C/C=C\CCCCCCCC(=O)OCC1(COC(=O)CCC(OCCCCCCCC)OCCCCCCCC)CCN(C(=O)OCCCN(CC)CC)CC1] | CX-15 |
| 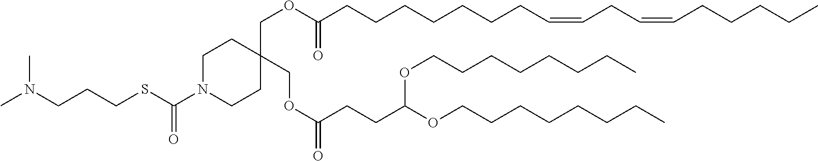 [SMILES: CCCCC/C=C\C/C=C\CCCCCCCC(=O)OCC1(COC(=O)CCC(OCCCCCCCC)OCCCCCCCC)CCN(C(=O)SCCCN(C)C)CC1] | CX-16 |
| 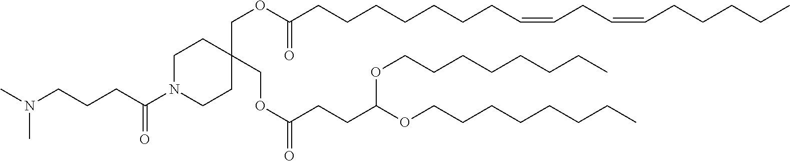 [SMILES: CCCCC/C=C\C/C=C\CCCCCCCC(=O)OCC1(COC(=O)CCC(OCCCCCCCC)OCCCCCCCC)CCN(C(=O)CCCN(C)C)CC1] | CX-17 |
| 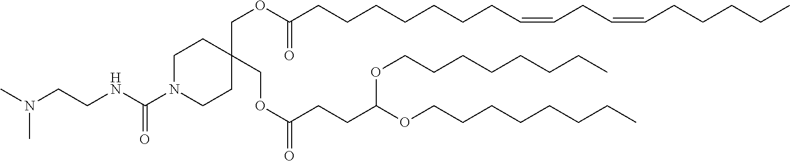 [SMILES: CCCCC/C=C\C/C=C\CCCCCCCC(=O)OCC1(COC(=O)CCC(OCCCCCCCC)OCCCCCCCC)CCN(C(=O)NCCN(C)C)CC1] | CX-18 |
| 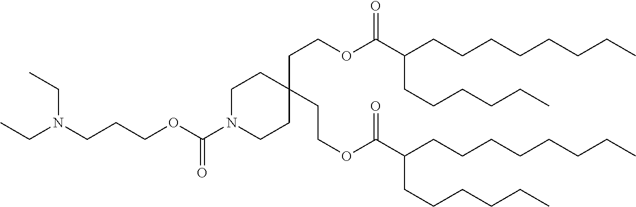 [SMILES: CCCCCCCCC(CCCCCC)C(=O)OCCC1(CCOC(=O)C(CCCCCC)CCCCCCCC)CCN(C(=O)OCCCN(CC)CC)CC1] | CX-19 |
| 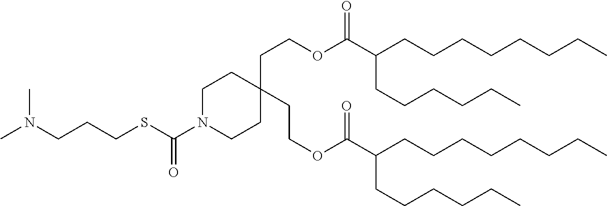 [SMILES: CCCCCCCCC(CCCCCC)C(=O)OCCC1(CCOC(=O)C(CCCCCC)CCCCCCCC)CCN(C(=O)SCCCN(C)C)CC1] | CX-20 |
| 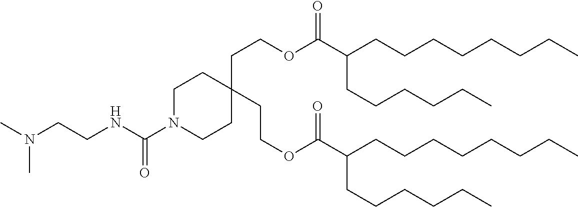 [SMILES: CCCCCCCCC(CCCCCC)C(=O)OCCC1(CCOC(=O)C(CCCCCC)CCCCCCCC)CCN(C(=O)NCCN(C)C)CC1] | CX-21 |
| 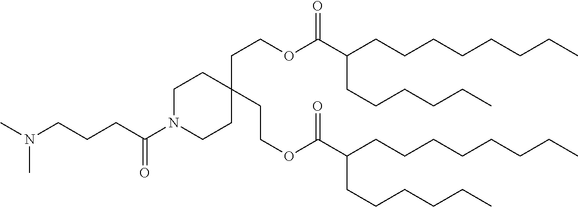 [SMILES: CCCCCCCCC(CCCCCC)C(=O)OCCC1(CCOC(=O)C(CCCCCC)CCCCCCCC)CCN(C(=O)CCCN(C)C)CC1] | CX-22 |
| 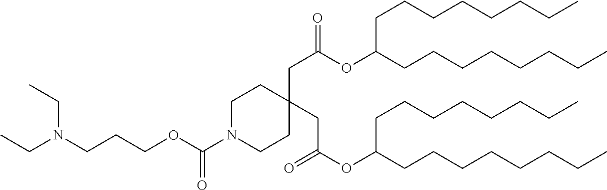 [SMILES: CCCCCCCCC(CCCCCCCC)OC(=O)CC1(CC(=O)OC(CCCCCCCC)CCCCCCCC)CCN(C(=O)OCCCN(CC)CC)CC1] | CX-23 |
| 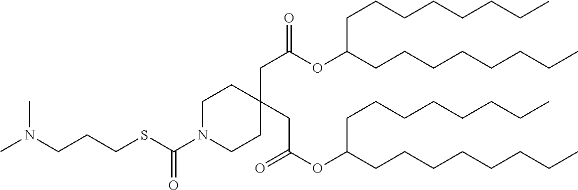 [SMILES: CCCCCCCCC(CCCCCCCC)OC(=O)CC1(CC(=O)OC(CCCCCCCC)CCCCCCCC)CCN(C(=O)SCCCN(C)C)CC1] | CX-24 |
| 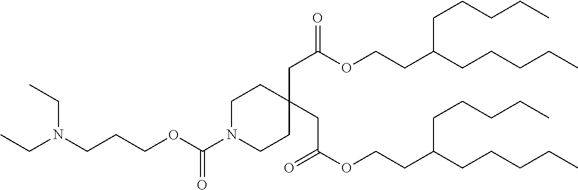 [SMILES: CCCCCC(CCCCC)CCOC(=O)CC1(CC(=O)OCCC(CCCCC)CCCCC)CCN(C(=O)OCCCN(CC)CC)CC1] | CX-25 |
| 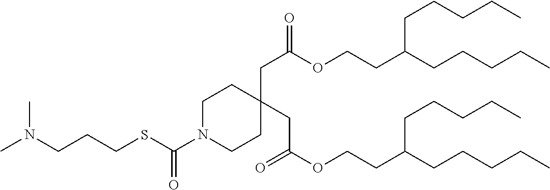 [SMILES: CCCCCC(CCCCC)CCOC(=O)CC1(CC(=O)OCCC(CCCCC)CCCCC)CCN(C(=O)SCCCN(C)C)CC1] | CX-26 |
| 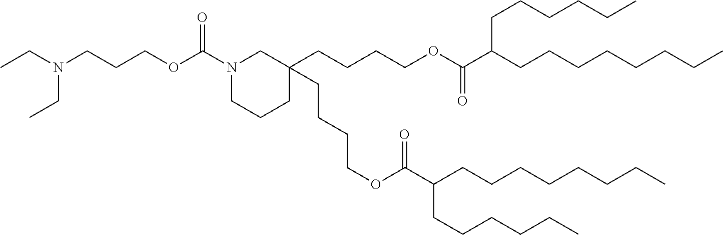 [SMILES: CCCCCCCCC(CCCCCC)C(=O)OCCCCC1(CCCCOC(=O)C(CCCCCC)CCCCCCCC)CCCN(C(=O)OCCCN(CC)CC)C1] | CX-27 |
| 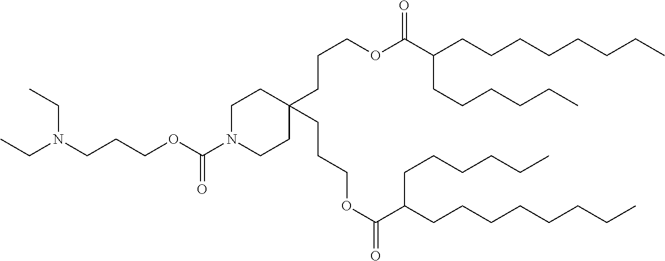 [SMILES: CCCCCCCCC(CCCCCC)C(=O)OCCCC1(CCCOC(=O)C(CCCCCC)CCCCCCCC)CCN(C(=O)OCCCN(CC)CC)CC1] | CX-28 |
| 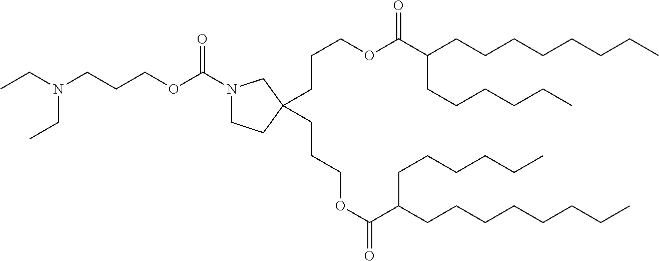 [SMILES: CCCCCCCCC(CCCCCC)C(=O)OCCCC1(CCCOC(=O)C(CCCCCC)CCCCCCCC)CCN(C(=O)OCCCN(CC)CC)C1] | CX-29 |
| 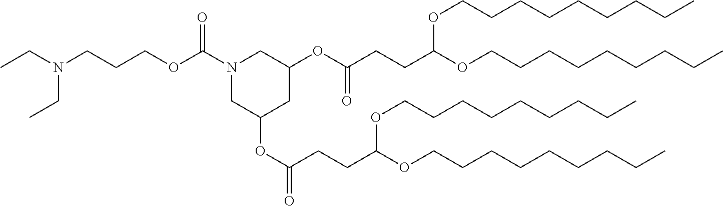 [SMILES: CCCCCCCCCOC(CCC(=O)OC1CC(OC(=O)CCC(OCCCCCCCCC)OCCCCCCCCC)CN(C(=O)OCCCN(CC)CC)C1)OCCCCCCCCC] | CX-30 |
| 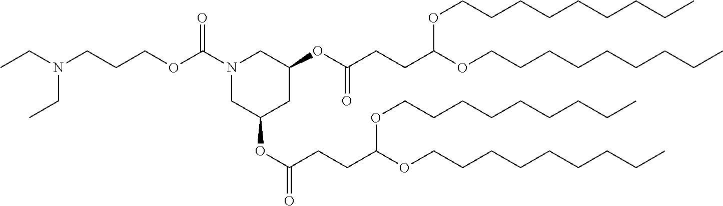 [SMILES: CCCCCCCCCOC(CCC(=O)O[C@@H]1C[C@H](OC(=O)CCC(OCCCCCCCCC)OCCCCCCCCC)CN(C(=O)OCCCN(CC)CC)C1)OCCCCCCCCC] | CX-30a |
| 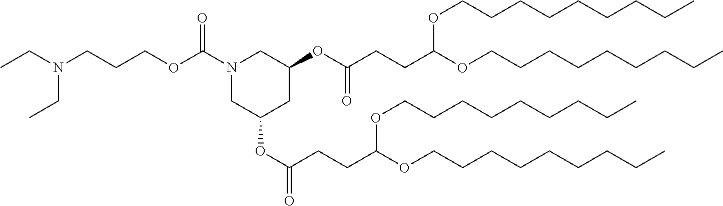 [SMILES: CCCCCCCCCOC(CCC(=O)O[C@H]1C[C@H](OC(=O)CCC(OCCCCCCCCC)OCCCCCCCCC)CN(C(=O)OCCCN(CC)CC)C1)OCCCCCCCCC] | CX-30b |
| 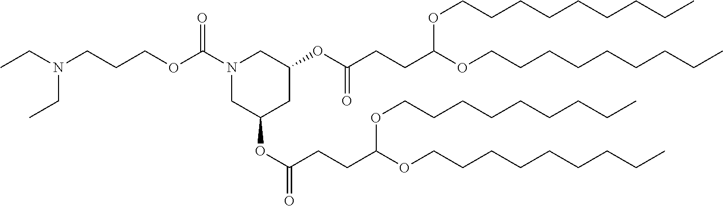 [SMILES: CCCCCCCCCOC(CCC(=O)O[C@@H]1C[C@@H](OC(=O)CCC(OCCCCCCCCC)OCCCCCCCCC)CN(C(=O)OCCCN(CC)CC)C1)OCCCCCCCCC] | CX-30c |

In some embodiments, lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

In some embodiments, a compound of the present disclosure is represented by Formula (CZ-I)

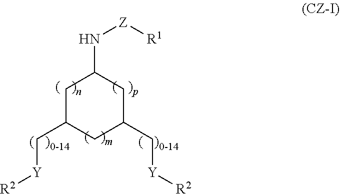

or a pharmaceutically acceptable salt thereof,
wherein
Z is selected from the group consisting of a bond,

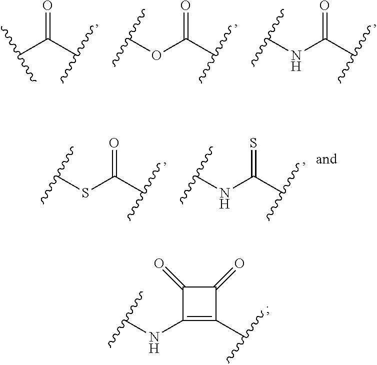

each Y is independently selected from the group consisting of,

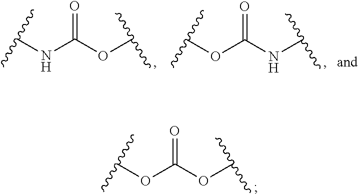 [SMILES: *NC(=O)O* ; *NC(=O)O* ; *OC(=O)O*]

R¹ is —(CH₂)₁₋₆N(R^{a})₂;
each R² is independently optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by Formula (CZ-I-a), (CZ-I-b), (CZ-I-c), or (CZ-I-d)

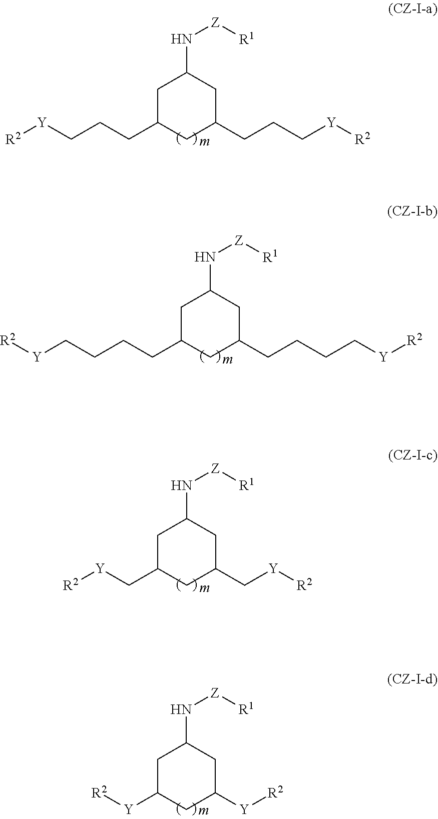

or a pharmaceutically acceptable salt thereof.

In some embodiments, a compound of the present disclosure is represented by Formula (CZ-I-e) or (CZ-I-f)

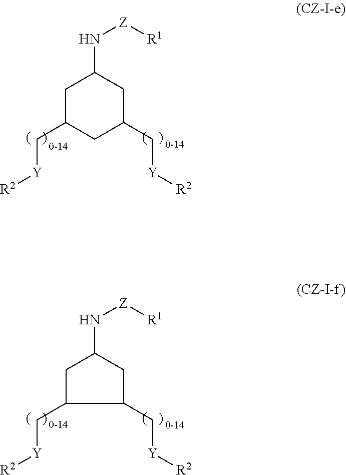

or a pharmaceutically acceptable salt thereof.

In some embodiments, a compound of the present disclosure is represented by Formula (CZ-I-g)

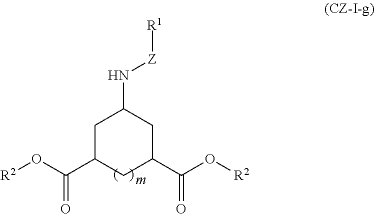

or a pharmaceutically acceptable salt thereof.

In some embodiments, a compound of the present disclosure is represented by Formula (CZ-II)

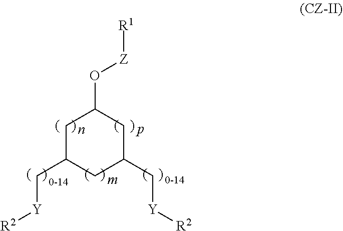

or a pharmaceutically acceptable salt thereof,

wherein
Z is selected from the group consisting of a bond,

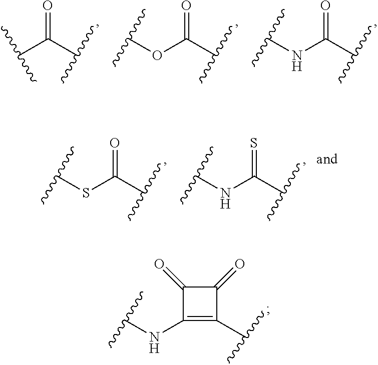

each Y is independently selected from the group consisting of,

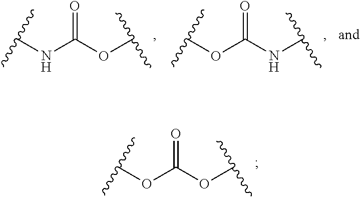 [SMILES: *NC(=O)O* ; *NC(=O)O* ; *OC(=O)O*]

R¹ is —(CH₂)₁₋₆N(R^{a})₂;
each R² is independently optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2;
n is 1 or 2; and
p is 1 or 2,
or a pharmaceutically acceptable salt thereof.

In some embodiments, a compound of the present disclosure is represented by Formula (CZ-II-a), (CZ-II-b), (CZ-II-c) or (CZ-II-d):

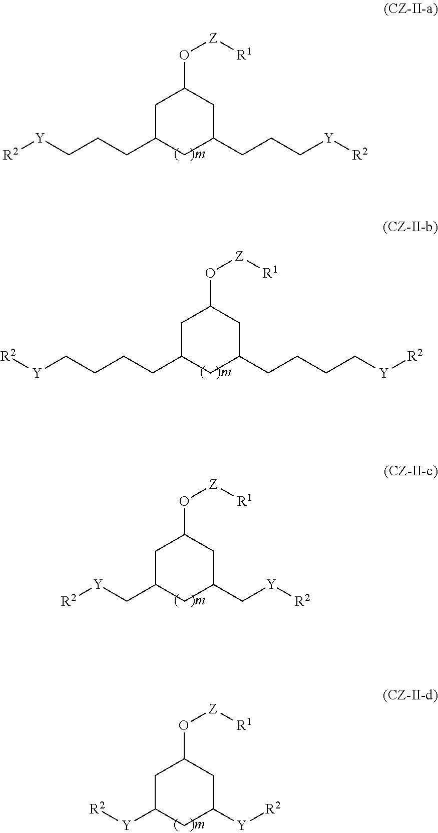

or a pharmaceutically acceptable salt thereof.

In some embodiments, a compound of the present disclosure is represented by Formula (CZ-II-e)

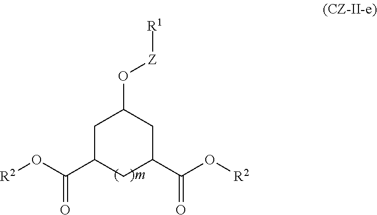

or a pharmaceutically acceptable salt thereof.

### Z

In some embodiments, Z is selected from the group consisting of a bond,

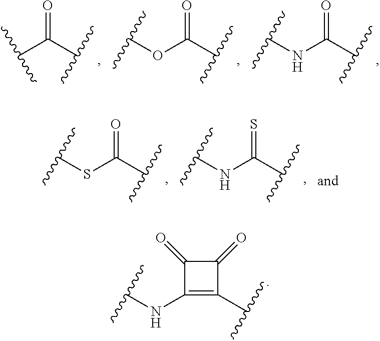

In some embodiments, Z is selected from the group consisting of

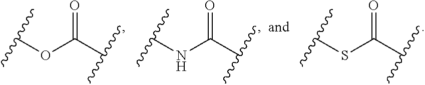 [SMILES: *OC(*)=O ; *NC(*)=O ; *SC(*)=O]

In some embodiments, Z is

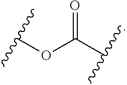 [SMILES: *OC(*)=O]

In some embodiments, Z is selected from the group consisting of a bond,

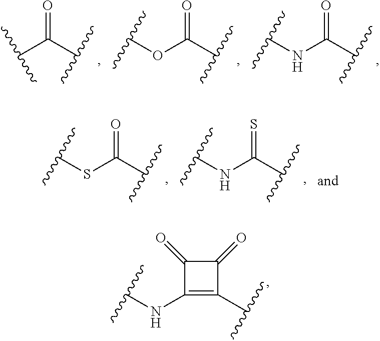

wherein R¹ is attached at the position denoted by *
In some embodiments, Z is selected from the group consisting of

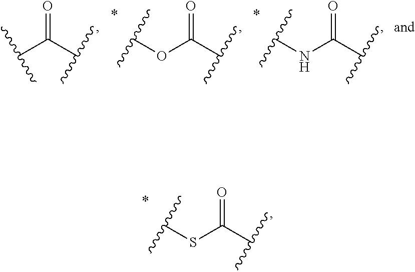

wherein R¹ is attached at the position denoted by *. In some embodiments, Z is

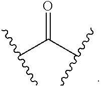 [SMILES: *C(*)=O]

In some embodiments, Z is wherein R¹ is

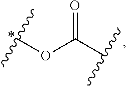

wherein R¹ is attached at the position denoted by *. In some embodiments, Z is

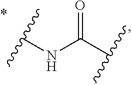

wherein R¹ is attached at the position denoted by *. In some embodiments, Z is

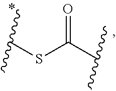

wherein R¹ is attached at the position denoted by *. In some embodiments, Z is

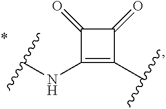

wherein R¹ is attached at the position denoted by *.

### Y

In some embodiments, Y is selected from the group consisting of,

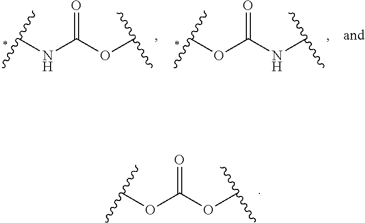 [SMILES: *NC(=O)O* ; *NC(=O)O* ; *OC(=O)O*]

In some embodiments, Y is

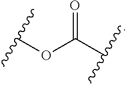 [SMILES: *OC(*)=O]

or.

In some embodiments, Y is selected from the group consisting of,

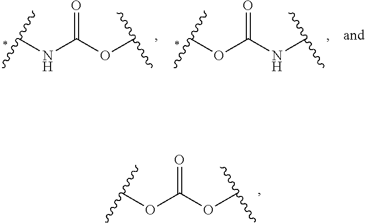

wherein R² is attached at the position denoted by * In some embodiments, Y is, wherein R² is attached at the position denoted by *. In some embodiments, Y is, wherein R² is attached at the position denoted by *. In some embodiments, Y is

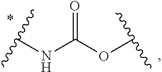

wherein R² is attached at the position denoted by *. In some embodiments, Y is

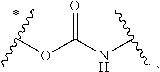

wherein R² is attached at the position denoted by * In some embodiments, Y is

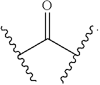 [SMILES: *C(*)=O]

In some embodiments, Y is

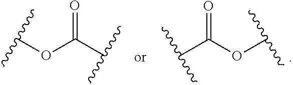 [SMILES: *OC(*)=O ; *OC(*)=O]

In some embodiments, Y is

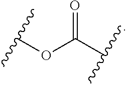 [SMILES: *OC(*)=O]

In some embodiments, Y is

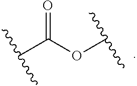 [SMILES: *OC(*)=O]

### R¹

In some embodiments, R¹ is —(CH₂)₁₋₆N(R^{a})₂. In some embodiments, R¹ is —(CH₂)₂N(R^{a})₂. In some embodiments, R¹ is —(CH₂)₃N(R^{a})₂. In some embodiments, R¹ is —(CH₂)₄N(R^{a})₂. In some embodiments, R¹ is —(CH₂)₁₋₆N(Me)₂. In some embodiments, R¹ is —(CH₂)₁₋₆N(Et)₂. In some embodiments, R¹ is —(CH₂)₁₋₆N(n-Pr)₂. In some embodiments, R¹ is (CH₂)₁₋₆N(CZ—I—Pr)₂. In some embodiments, R¹ is —(CH₂)₂N(Me)₂. In some embodiments, R¹ is —(CH₂)₃N(Me)₂. In some embodiments, R¹ is —(CH₂)₄N(Me)₂. In some embodiments, R¹ is —(CH₂)₂N(Et)₂. In some embodiments, R¹ is —(CH₂)₃N(Et)₂. In some embodiments, R¹ is —(CH₂)₄N(Et)₂.

In some embodiments, R¹ is selected from the group consisting of

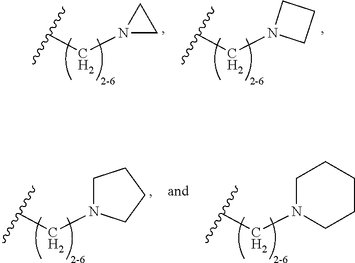

In some embodiments, R¹ is selected from the group consisting of

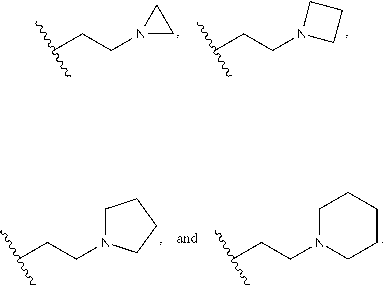

In some embodiments, R¹ is selected from the group consisting of

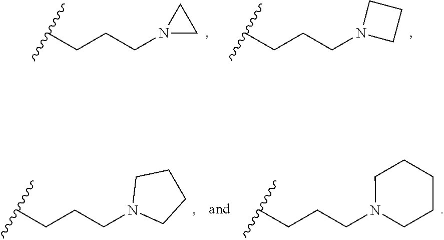 [SMILES: *CCCN1CC1 ; *CCCN1CCC1 ; *CCCN1CCCC1 ; *CCCN1CCCCC1]

### R²

In some embodiments, R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—. In some embodiments, R² is optionally substituted C₁-C₃₂ alkyl or optionally substituted C₂-C₃₂ alkenyl, wherein 1-6 methylene units of R² are optionally replaced with a group each independently selected from cyclopropylene, —O—, —OC(O)—, and —C(O)O—. In some embodiments, R² is optionally substituted C₁-C₃₀ alkyl or optionally substituted C₂-C₃₀ alkenyl, wherein 1-6 methylene units of R² are optionally replaced with a group each independently selected from cyclopropylene, —O—, —OC(O)—, and —C(O)O—. In some embodiments, R² is optionally substituted C₁-C₂₄ alkyl or optionally substituted C₂-C₂₄ alkenyl, wherein 1-6 methylene units of R² are optionally replaced with a group each independently selected from cyclopropylene, —O—, —OC(O)—, and —C(O)O—. In some embodiments, R² is optionally substituted C₁-C₂₄ alkyl or optionally substituted C₂-C₂₄ alkenyl, wherein 1-6 methylene units of R² are replaced with a group each independently selected from —O—, —OC(O)—, and —C(O)O—. In some embodiments, R² is optionally substituted C₁-C₂₄ alkyl or optionally substituted C₂-C₂₄ alkenyl. In some embodiments, R² is optionally substituted C₁₀-C₂₄ alkyl or optionally substituted C₁₀-C₂₄ alkenyl, wherein 1-6 methylene units of R² are replaced with —O—.

In some embodiments, R² is

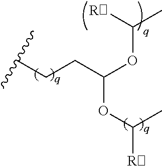

wherein each q is independently selected from 0-12 and each R° is independently selected and defined herein.

In some embodiments, R² is

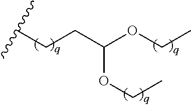

wherein each q is independently selected from 0-12.

In some embodiments, each q is independently selected from 0-6. In some embodiments, each q is independently selected from 0-8. In some embodiments, each q is independently selected from 0-10. In some embodiments, each q is independently selected from 0-12.

In some embodiments, R² is selected from the group consisting of

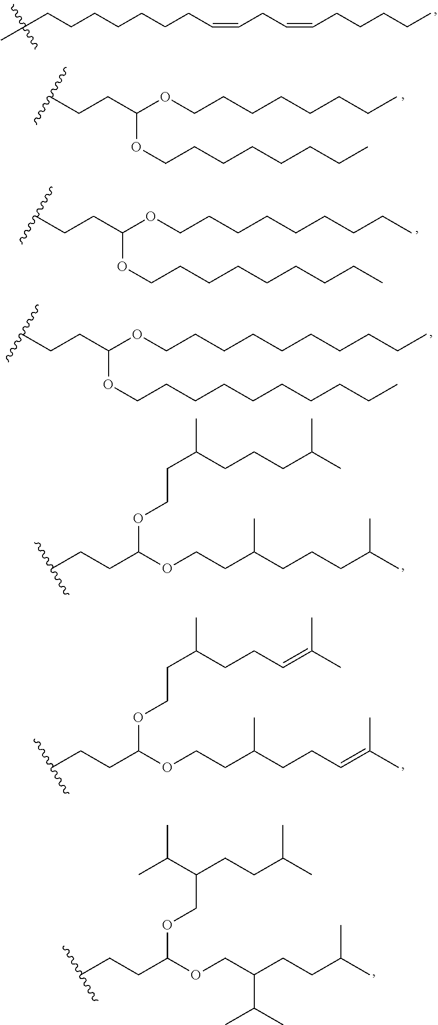

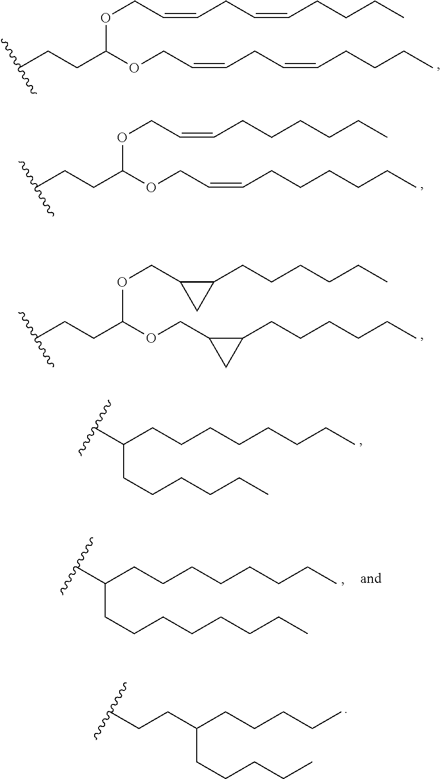 [SMILES: *CCC(OC/C=C\C/C=C\CCCC)OC/C=C\C/C=C\CCCC ; *CCC(OC/C=C\CCCCCC)OC/C=C\CCCCCC ; *CCC(OCC1CC1CCCCCC)OCC1CC1CCCCCC ; *C(CCCCCC)CCCCCCCC ; *C(CCCCCCCC)CCCCCCCC ; *CCC(CCCCC)CCCCC]

In some embodiments, R² is selected from the group consisting of

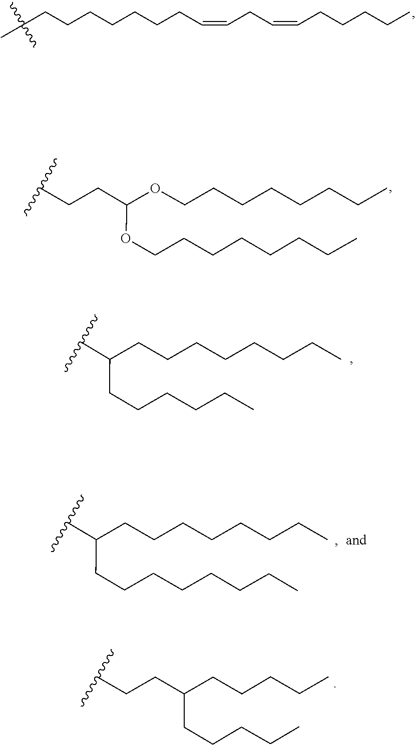 [SMILES: *CCCCCCC/C=C\C/C=C\CCCCC ; *CCC(OCCCCCCCC)OCCCCCCCC ; *C(CCCCCC)CCCCCCCC ; *C(CCCCCCCC)CCCCCCCC ; *CCC(CCCCC)CCCCC]

In some embodiments, the present disclosure includes a compound selected from any lipid in Table (V) below or a pharmaceutically acceptable salt thereof:

TABLE (V)
Non-Limiting Examples of Ionizable Lipids

| | Compound |
|---|---|
| Structure | No. |
| 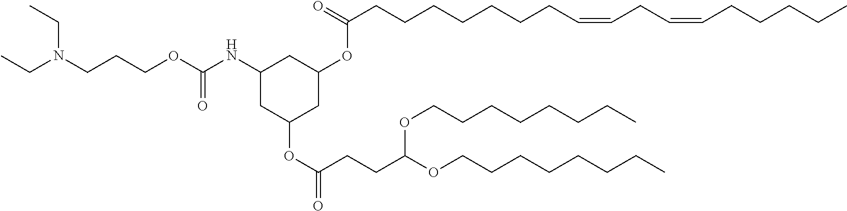 [SMILES: CCCCC/C=C\C/C=C\CCCCCCCC(=O)OC1CC(NC(=O)OCCCN(CC)CC)CC(OC(=O)CCC(OCCCCCCCC)OCCCCCCCC)C1] | CZ-1 |
| 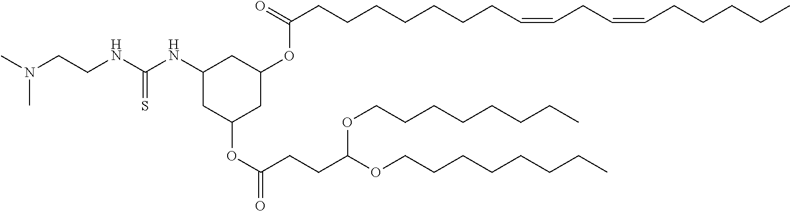 [SMILES: CCCCC/C=C\C/C=C\CCCCCCCC(=O)OC1CC(NC(=S)NCCN(C)C)CC(OC(=O)CCC(OCCCCCCCC)OCCCCCCCC)C1] | CZ-2 |
| 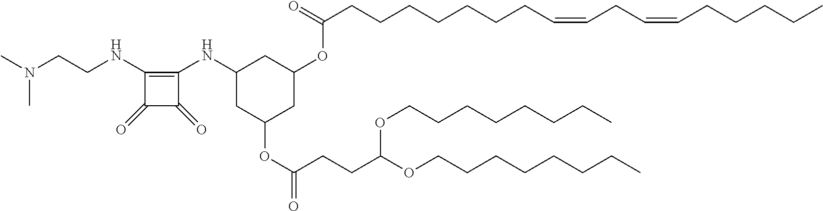 [SMILES: CCCCC/C=C\C/C=C\CCCCCCCC(=O)OC1CC(Nc2c(NCCN(C)C)c(=O)c2=O)CC(OC(=O)CCC(OCCCCCCCC)OCCCCCCCC)C1] | CZ-3 |
| 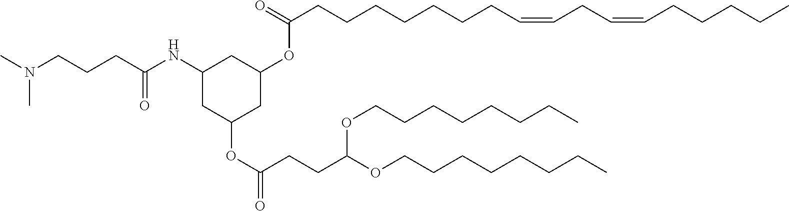 [SMILES: CCCCC/C=C\C/C=C\CCCCCCCC(=O)OC1CC(NC(=O)CCCN(C)C)CC(OC(=O)CCC(OCCCCCCCC)OCCCCCCCC)C1] | CZ-4 |
| 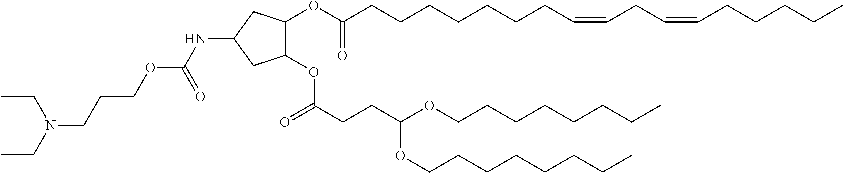 [SMILES: CCCCC/C=C\C/C=C\CCCCCCCC(=O)OC1CC(NC(=O)OCCCN(CC)CC)CC1OC(=O)CCC(OCCCCCCCC)OCCCCCCCC] | CZ-5 |
| 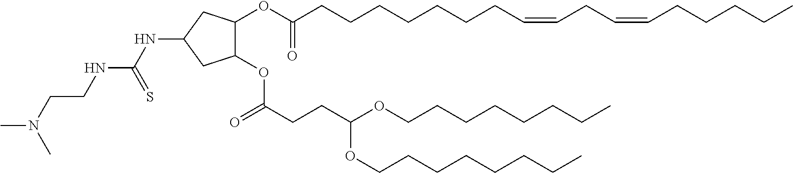 [SMILES: CCCCC/C=C\C/C=C\CCCCCCCC(=O)OC1CC(NC(=S)NCCN(C)C)CC1OC(=O)CCC(OCCCCCCCC)OCCCCCCCC] | CZ-6 |
| 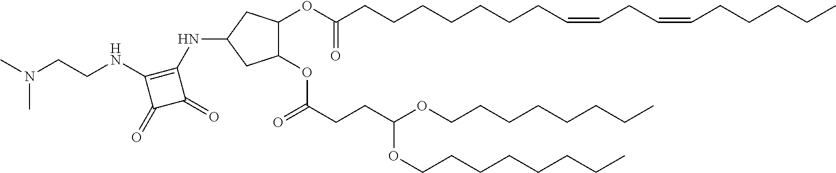 [SMILES: CCCCC/C=C\C/C=C\CCCCCCCC(=O)OC1CC(Nc2c(NCCN(C)C)c(=O)c2=O)CC1OC(=O)CCC(OCCCCCCCC)OCCCCCCCC] | CZ-7 |
| 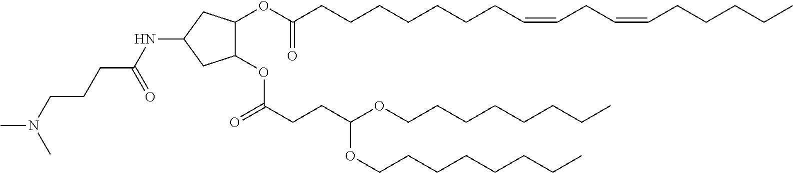 [SMILES: CCCCC/C=C\C/C=C\CCCCCCCC(=O)OC1CC(NC(=O)CCCN(C)C)CC1OC(=O)CCC(OCCCCCCCC)OCCCCCCCC] | CZ-8 |
| 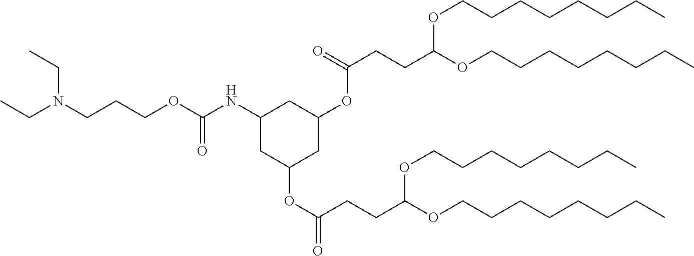 [SMILES: CCCCCCCCOC(CCC(=O)OC1CC(NC(=O)OCCCN(CC)CC)CC(OC(=O)CCC(OCCCCCCCC)OCCCCCCCC)C1)OCCCCCCCC] | CZ-9 |
| 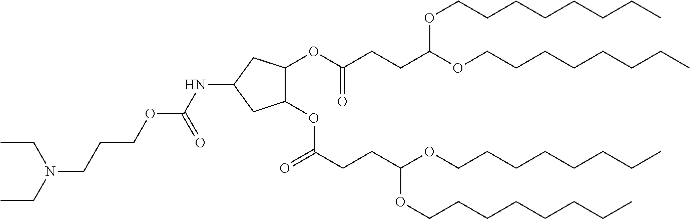 [SMILES: CCCCCCCCOC(CCC(=O)OC1CC(NC(=O)OCCCN(CC)CC)CC1OC(=O)CCC(OCCCCCCCC)OCCCCCCCC)OCCCCCCCC] | CZ-10 |
| 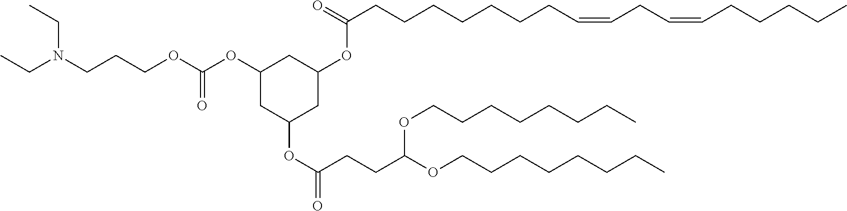 [SMILES: CCCCC/C=C\C/C=C\CCCCCCCC(=O)OC1CC(OC(=O)CCC(OCCCCCCCC)OCCCCCCCC)CC(OC(=O)OCCCN(CC)CC)C1] | CZ-11 |
| 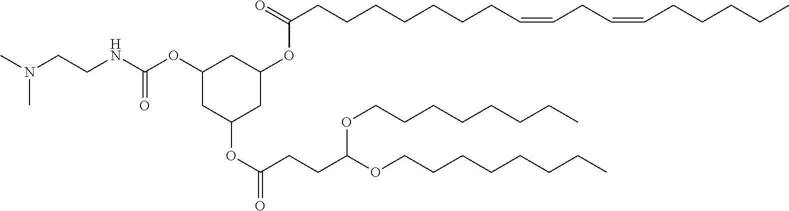 [SMILES: CCCCC/C=C\C/C=C\CCCCCCCC(=O)OC1CC(OC(=O)CCC(OCCCCCCCC)OCCCCCCCC)CC(OC(=O)NCCN(C)C)C1] | CZ-12 |
| 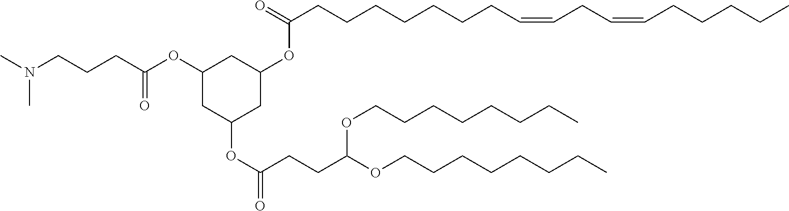 [SMILES: CCCCC/C=C\C/C=C\CCCCCCCC(=O)OC1CC(OC(=O)CCCN(C)C)CC(OC(=O)CCC(OCCCCCCCC)OCCCCCCCC)C1] | CZ-13 |
| 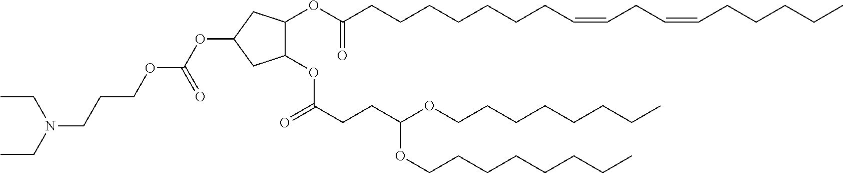 [SMILES: CCCCC/C=C\C/C=C\CCCCCCCC(=O)OC1CC(OC(=O)OCCCN(CC)CC)CC1OC(=O)CCC(OCCCCCCCC)OCCCCCCCC] | CZ-14 |
| 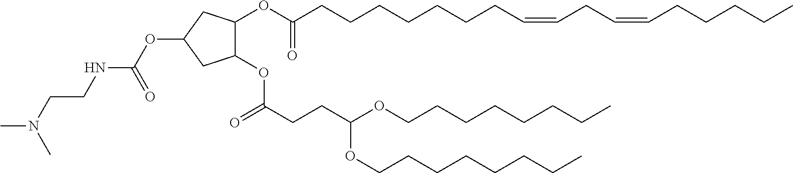 [SMILES: CCCCC/C=C\C/C=C\CCCCCCCC(=O)OC1CC(OC(=O)NCCN(C)C)CC1OC(=O)CCC(OCCCCCCCC)OCCCCCCCC] | CZ-15 |
| 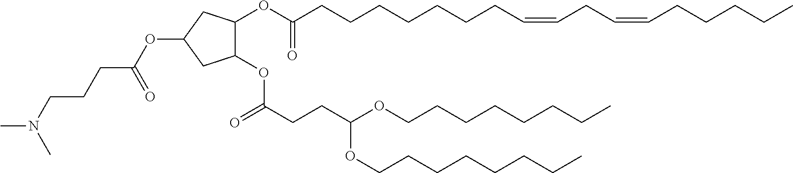 [SMILES: CCCCC/C=C\C/C=C\CCCCCCCC(=O)OC1CC(OC(=O)CCCN(C)C)CC1OC(=O)CCC(OCCCCCCCC)OCCCCCCCC] | CZ-16 |
| 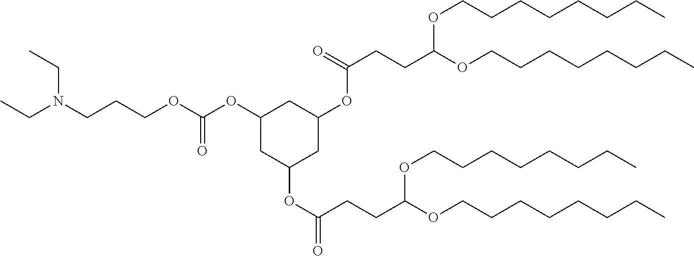 [SMILES: CCCCCCCCOC(CCC(=O)OC1CC(OC(=O)CCC(OCCCCCCCC)OCCCCCCCC)CC(OC(=O)OCCCN(CC)CC)C1)OCCCCCCCC] | CZ-17 |
| 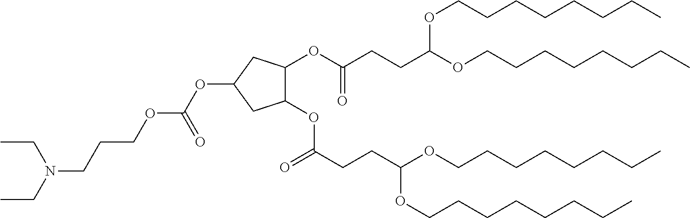 [SMILES: CCCCCCCCOC(CCC(=O)OC1CC(OC(=O)OCCCN(CC)CC)CC1OC(=O)CCC(OCCCCCCCC)OCCCCCCCC)OCCCCCCCC] | CZ-18 |

ii. Structural Lipids

In some embodiments, an LNP comprises a structural lipid. Structural lipids can be selected from the group consisting of, but are not limited to, cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate and mixtures thereof. In some embodiments, the structural lipid is cholesterol. In some embodiments, the structural lipid is a cholesterol analogue disclosed by Patel, et al., Nat Commun., 11, 983 (2020), which is incorporated herein by reference in its entirety. In some embodiments, the structural lipid includes cholesterol and a corticosteroid (such as prednisolone, dexamethasone, prednisone, and hydrocortisone), or any combinations thereof. In some embodiments, a structural lipid is described in international patent application WO2019152557A1, which is incorporated herein by reference in its entirety.

In some embodiments, a structural lipid is a cholesterol analog. Using a cholesterol analog may enhance endosomal escape as described in Patel et al., Naturally-occurring cholesterol analogues in lipid nanoparticles induce polymorphic shape and enhance intracellular delivery of mRNA, Nature Communications (2020), which is incorporated herein by reference.

In some embodiments, a structural lipid is a phytosterol. Using a phytosterol may enhance endosomal escape as described in Herrera et al., Illuminating endosomal escape of polymorphic lipid nanoparticles that boost mRNA delivery, Biomaterials Science (2020), which is incorporated herein by reference.

In some embodiments, a structural lipid contains plant sterol mimetics for enhanced endosomal release.

iii. PEGylated Lipids

A PEGylated lipid is a lipid modified with polyethylene glycol.

In some embodiments, an LNP comprises one, two or more PEGylated lipid or PEG-modified lipid. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, the PEGylated lipid is selected from (R)-2,3-bis (octadecyloxy) propyl-1-(methoxypoly(ethyleneglycol)2000) propylcarbamate, PEG-S— DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-CDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG (2k)-DMG, PEG DSPE C18, PEG DMPE C14, PEG DLPE C12, PEG Click DMG C14, PEG Click C12, PEG Click C10, N(Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, CI8PEG5000, CI8PEG3000, CI8PEG2000, CI6PEG2000, CI4PEG2000, C18-PEG5000, C18PEG, C16PEG, C16 mPEG (polyethylene glycol)2000 Ceramide, C14-PEG-DSPE200, C14-PEG2000, C14PEG2000, C14-PEG 2000, C14-PEG, C14PEG, 14:0-PEG2KPE, 1,2-distearoyl-sn-glycero-3-phosphoethanolamine-PEG2000, (R)-2,3-bis (octadecyloxy) propyl-1-(methoxypoly(ethyleneglycol)2000) propylcarbamate, (PEG)-C-DOMG, PEG-C-DMA, and DSPE-PEG-X.

In some embodiments, the LNP comprises a PEGylated lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; or WO 2019/089828A1, each of which is incorporated by reference herein in their entirety.

In some embodiments, the LNP comprises a PEGylated lipid substitute in place of the PEGylated lipid. All embodiments disclosed herein that contemplate a PEGylated lipid should be understood to also apply to PEGylated lipid substitutes. In some embodiments, the LNP comprises a polysarcosine-lipid conjugate, such as those disclosed in US 2022/0001025 A1, which is incorporated by reference herein in its entirety.

iv. Phospholipids

In some embodiments, an LNP of the present disclosure comprises a phospholipid. Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium(S)-2-ammonio-3-((((R)-2-(oleoyloxy)-3-(stearoyloxy) propoxy) oxidophosphoryl)oxy) propanoate (L-α-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine)(DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy) propyl) phosphate (18:1 DMP; LBPA), 1,2-dioleoyl-sn-glycero-3-phospho-(1'-myo-inositol)(DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin. In some embodiments, an LNP includes DSPC. In certain embodiments, an LNP includes DOPE. In some embodiments, an LNP includes both DSPC and DOPE.

In some embodiments, an LNP comprises a phospholipid selected from 1-pentadecanoyl-2-oleoyl-sn-glycero-3-phosphocholine, 1-myristoyl-2-palmitoyl-sn-glycero-3-phosphocholine, 1-myristoyl-2-stearoyl-sn-glycero-3-phosphocholine, 1-palmitoyl-2-myristoyl-sn-glycero-3-phosphocholine, 1-palmitoyl-2-stearoyl-sn-glycero-3-phosphocholine, 1-palmitoyl-2-oleoyl-glycero-3-phosphocholine, 1-palmitoyl-2-linoleoyl-sn-glycero-3-phosphocholine, 1-palmitoyl-2-arachidonoyl-sn-glycero-3-phosphocholine, 1-palmitoyl-2-docosahexaenoyl-sn-glycero-3-phosphocholine, 1-stearoyl-2-myristoyl-sn-glycero-3-phosphocholine, 1-stearoyl-2-palmitoyl-sn-glycero-3-phosphocholine, 1-stearoyl-2-oleoyl-sn-glycero-3-phosphocholine, 1-stearoyl-2-linoleoyl-sn-glycero-3-phosphocholine, 1-stearoyl-2-arachidonoyl-sn-glycero-3-phosphocholine, 1-stearoyl-2-docosahexaenoyl-sn-glycero-3-phosphocholine, 1-oleoyl-2-myristoyl-sn-glycero-3-phosphocholine, 1-oleoyl-2-palmitoyl-sn-glycero-3-phosphocholine, 1-oleoyl-2-stearoyl-sn-glycero-3-phosphocholine, 1-palmitoyl-2-acetyl-sn-glycero-3-phosphocholine, 1,2-dioleoyl-sn-glycero-3-phospho-(1′-myo-inositol-3′, 4′-bisphosphate), 1,2-dioleoyl-sn-glycero-3-phospho-(1′-myo-inositol-3′, 5′-bisphosphate), 1,2-dioleoyl-sn-glycero-3-phospho-(1′-myo-inositol-4′, 5′-bisphosphate), 1,2-dioleoyl-sn-glycero-3-phospho-(1′-myo-inositol-3′, 4′, 5′-trisphosphate), 1,2-dioleoyl-sn-glycero-3-phospho-(1′-myo-inositol-3′-phosphate), 1,2-dioleoyl-sn-glycero-3-phospho-(1′-myo-inositol-4′-phosphate), 1,2-dioleoyl-sn-glycero-3-phospho-(1′-myo-inositol-5′-phosphate), 1,2-dioleoyl-sn-glycero-3-phospho-(1′-myo-inositol), 1,2-dioleoyl-sn-glycero-3-phospho-L-serine, and 1-(8Z-octadecenoyl)-2-palmitoyl-sn-glycero-3-phosphocholine.

In some embodiments, a phospholipid tail may be modified in order to promote endosomal escape as described in U.S. Application Publication 2021/0121411, which is incorporated herein by reference.

In some embodiments, the LNP comprises a phospholipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, phospholipids disclosed in US 2020/0121809 have the following structure:

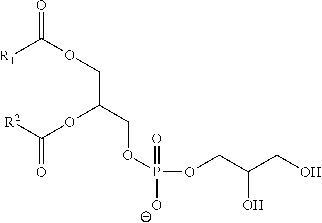

wherein R1 and R2 are each independently a branched or straight, saturated or unsaturated carbon chain (e.g., alkyl, alkenyl, alkynyl).
vi. Targeting Moieties

In some embodiments, the lipid nanoparticle further comprises a targeting moiety. The targeting moiety may be an antibody or a fragment thereof. The targeting moiety may be capable of binding to a target antigen.

In some embodiments, the pharmaceutical composition comprises a targeting moiety that is operably connected to a lipid nanoparticle. In some embodiments, the targeting moiety is capable of binding to a target antigen. In some embodiments, the target antigen is expressed in a target organ. In some embodiments, the target antigen is expressed more in the target organ than it is in the liver.

In some embodiments, the targeting moiety is an antibody as described in WO2016189532A1, which is incorporated herein by reference. For example, in some embodiments, the targeted particles are conjugated to a specific anti-CD38 monoclonal antibody (mAb), which allows specific delivery of the siRNAs encapsulated within the particles at a greater percentage to B-cell lymphocytes malignancies (such as MCL) than to other subtypes of leukocytes.

In some embodiments, the lipid nanoparticles may be targeted when conjugated/attached/associated with a targeting moiety such as an antibody. vii. Zwitterionic amino lipids

In some embodiments, an LNP comprises a zwitterionic lipid. In some embodiments, an LNP comprising a zwitterionic lipid does not comprise a phospholipid.

Zwitterionic amino lipids have been shown to be able to self-assemble into LNPs without phospholipids to load, stabilize, and release mRNAs intracellularly as described in U.S. patent application No. 20210121411, which is incorporated herein by reference in its entirety. Zwitterionic, ionizable cationic and permanently cationic helper lipids enable tissue-selective mRNA delivery and CRISPR-Cas9 gene editing in spleen, liver and lungs as described in Liu et al., Membrane-destablizing ionizable phospholipids for organ-selective mRNA delivery and CRISPR-Cas gene editing, Nat Mater. (2021), which is incorporated herein by reference in its entirety.

The zwitterionic lipids may have head groups containing a cationic amine and an anionic carboxylate as described in Walsh et al., Synthesis, Characterization and Evaluation of Ionizable Lysine-Based Lipids for siRNA Delivery, Bioconjug Chem. (2013), which is incorporated herein by reference in its entirety. Ionizable lysine-based lipids containing a lysine head group linked to a long-chain dialkylamine through an amide linkage at the lysine α-amine may reduce immunogenicity as described in Walsh et al., Synthesis, Characterization and Evaluation of Ionizable Lysine-Based Lipids for siRNA Delivery, Bioconjug Chem. (2013).

viii. Additional Lipid Components

In some embodiments, the LNP compositions of the present disclosure further comprise one or more additional lipid components capable of influencing the tropism of the LNP. In some embodiments, the LNP further comprises at least one lipid selected from DDAB, EPC, 14PA, 18BMP, DODAP, DOTAP, and C12-200 (see Cheng, et al. Nat Nanotechnol. 2020 April; 15 (4): 313-320.; Dillard, et al. PNAS 2021 Vol. 118 No. 52.).

In some embodiments, the LNP compositions of the present disclosure comprise, or further comprise one or more lipids selected from 1,2-di-O-octadecenyl-sn-glycero-3-phosphocholine (18:0 Diether PC), 1,2-dilinolenoyl-sn-glycero-3-phosphocholine (18:3 PC), Acylcarnosine (AC), 1-hexadecyl-sn-glycero-3-phosphocholine (C16 Lyso PC), N-oleoyl-sphingomyelin (SPM)(C18: 1), N-lignoceryl SPM (C24: 0), N-nervonoylshphingomyelin (C24: 1), Cardiolipin (CL), 1,2-bis (tricosa-10,12-diynoyl)-sn-glycero-3-phosphocholine (DC8-9PC), dicetyl phosphate (DCP), dihexadecyl phosphate (DCP1), 1,2-Dipalmitoylglycerol-3-hemisuccinate (DGSucc), short-chain bis -n-heptadecanoyl phosphatidylcholine (DHPC), dihexadecoyl-phosphoethanolamine (DHPE), 1,2-dilinoleoyl-sn-glycero-3-phosphocholine (DLPC), 1,2-dilauroyl-sn-glycero-3-PE (DLPE), dimyristoyl glycerol hemisuccinate (DMGS), dimyristoyl phosphatidylcholine (DMPC), dimyristoyl phosphoethanolamine (DMPE), dimyristoylphosphatidylglycerol (DMPG), dioleyloxybenzylalcohol (DOBA), 1,2-dioleoylglyceryl-3-hemisuccinate (DOGHEMS), N-[2-(2-{2-[2-(2,3-Bis -octadec-9-enyloxy-propoxy)-ethoxy]-ethoxy}-ethoxy)-ethyl]-3-(3,4,5-1rihydroxy-6-hydroxymethyl-letrahydro-pyran-2-ylsulfanyl)-propionamide (DOGP4aMan), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), histaminedistearoylglycerol (HDSG), 1,2-Dipalmitoylglycerol-hemisuccinate-Na-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-O-α-histidinyl-Na-hemisuccinate (IsohistsuccDG), mannosialized dipalmitoylphosphatidylethanolamine (ManDOG), 1,2-Dioleoyl-sn-Glycero-3-Phosphoethanolamine-N-[4-(p-maleimidomethyl) cyclohexane-carboxamide](MCC-PE), 1,2-diphytanoyl-sn-glycero-3-phosphoethanolamine (ME 16:0 PE), 1-myristoyl-2-hydroxy-sn-glycero-phosphocholine (MHPC), a thiol-reactive maleimide headgroup lipid e.g. 1,2-dioleoyl-sn-glycero-3-phosphoethanolamine-N-[4-(p-maleimidophenyl) but-yramid (MPB-PE), Nervonic Acid (NA), sodium cholate (NaChol), 1,2-dioleoyl-sn-glycero-3-[phosphoethanolamine-N-dodecanoyl (NC12-DOPE), 1-oleoyl-2-cholesteryl hemisuccinoyl-sn-glycero-3-phosphocholine (OChemsPC), phosphatidylethanolamine lipid (PE), PE lipid conjugated with polyethylene glycol (PEG)(e.g., polyethylene glycol-distearoylphosphatidylethanolamine lipid (PEG-PE)), phosphatidylglycerol (PG), partially hydrogenated soy phosphatidylchloline (PHSPC), phosphatidylinositol lipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), lissamine rhodamine B-phosphatidylethanolamine lipid (Rh-PE), purified soy-derived mixture of phospholipids (SIOO), phosphatidylcholine (SM), 18-1-trans-PE, 1-stearoyl-2-oleoyl-phosphatidyethanolamine (SOPE), soybean phosphatidylcholine (SPC), sphingomyelins (SPM), alpha, alpha-trehalose-6,6′-dibehenate (TDB), 1,2-dielaidoyl-sn-glycero-3-phophoethanolamine (transDOPE), ((23S,5R)-3-(bis (hexadecyloxy) methoxy)-5-(5-methyl-2,4-dioxo-3,4-dihydropyrimidin-1 (2H)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE, 16-O-dimethyl PE, and dioleylphosphatidylethanolamine

### 3. Payloads

### Nucleic Acid Payloads

In various embodiments, the TnpB editing compositions described herein can include a nucleic acid or polynucleotide payload, e.g., a linear or circular mRNA. For example, the TnpB gene editing systems may comprise one or more coding mRNA (circular or linear) for encoding TnpB and other accessory proteins and these RNA components may be delivered by LNPs.

In some embodiments, a LNP is capable of delivering a polynucleotide to a target cell, tissue, or organ. A polynucleotide, in its broadest sense of the term, includes any compound and/or substance that is or can be incorporated into an oligonucleotide chain. Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. RNAs useful in the compositions and methods described herein can be selected from the group consisting of but are not limited to, shortimers, antagomirs, antisense, ribozymes, short interfering RNA (siRNA), asymmetrical interfering RNA (aiRNA), microRNA (miRNA), Dicer substrate RNA (dsRNA), short hairpin RNA (shRNA), transfer RNA (tRNA), messenger RNA (mRNA), and mixtures thereof. In some embodiments, a polynucleotide is mRNA. In some embodiments, a polynucleotide is circular RNA. In some embodiments, a polynucleotide encodes a protein, e.g., a nucleobase editing enzyme. A polynucleotide may encode any polypeptide of interest, including any naturally or non-naturally occurring or otherwise modified polypeptide. A polypeptide may be of any size and may have any secondary structure or activity. In some embodiments, a polypeptide encoded by an mRNA may have a therapeutic effect when expressed in a cell.

In other embodiments, a polynucleotide is an siRNA. An siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. An siRNA may comprise a sequence that is complementary to an mRNA sequence that encodes a gene or protein of interest. In some embodiments, the siRNA may be an immunomodulatory siRNA.

In some embodiments, a polynucleotide is an shRNA or a vector or plasmid encoding the same. An shRNA may be produced inside a target cell upon delivery of an appropriate construct to the nucleus. Constructs and mechanisms relating to shRNA are well known in the relevant arts.

A polynucleotide may include a first region of linked nucleosides encoding a polypeptide of interest (e.g., a coding region), a first flanking region located at 5′-terminus of the first region (e.g., a 5′-UTR), a second flanking region located at 3′-terminus of the first region (e.g., a 3′-UTR), at least one 5′-cap region, and a 3′-stabilizing region. In some embodiments, a polynucleotide further includes a poly-A region or a Kozak sequence (e.g., in 5′-UTR). In some cases, polynucleotides may contain one or more intronic nucleotide sequences capable of being excised from the polynucleotide. In some embodiments, a polynucleotide (e.g., an mRNA) may include a 5′cap structure, a chain terminating nucleotide, a stem loop, a polyA sequence, and/or a polyadenylation signal. Any one of the regions of a nucleic acid may include one or more alternative components (e.g., an alternative nucleoside). For example, 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl pseudouridine or 1-ethyl-pseudouridine), and/or a 5-substituted cytidine (e.g., 5-methyl-cytidine). In some embodiments, a polynucleotide contains only naturally occurring nucleosides.

In some cases, a polynucleotide is greater than 30 nucleotides in length. In another embodiment, the poly nucleotide molecule is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 50 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1800 nucleotides. In another embodiment, the length is at least 2000 nucleotides. In another embodiment, the length is at least 2500 nucleotides. In another embodiment, the length is at least 3000 nucleotides. In another embodiment, the length is at least 4000 nucleotides. In another embodiment, the length is at least 5000 nucleotides, or greater than 5000 nucleotides.

In some embodiments, a polynucleotide molecule, formula, composition or method associated therewith comprises one or more polynucleotides comprising features as described in WO2002/098443, WO2003/051401, WO2008/052770, WO2009/127230, WO2006/122828, WO2008/083949, WO2010/088927, WO2010/037539, WO2004/004743, WO2005/016376, WO2006/024518, WO2007/095976, WO2008/014979, WO2008/077592, WO2009/030481, WO2009/095226, WO2011/069586, WO2011/026641, WO2011/144358, WO2012/019780, WO2012/013326, WO2012/089338, WO2012/113513, WO2012/116811, WO2012/116810, WO2013/113502, WO2013/113501, WO2013/113736, WO2013/143698, WO2013/143699, WO2013/143700, WO2013/120626, WO2013/120627, WO2013/120628, WO2013/120629, WO2013/174409, WO2014/127917, WO2015/024669, WO2015/024668, WO2015/024667, WO2015/024665, WO2015/024666, WO2015/024664, WO2015/101415, WO2015/101414, WO2015/024667, WO2015/062738, WO2015/101416, all of which are incorporated by reference herein.

In some embodiments, a polynucleotide comprises one or more microRNA binding sites. In some embodiments, a microRNA binding site is recognized by a microRNA in a non-target organ. In some embodiments, a microRNA binding site is recognized by a microRNA in the liver. In some embodiments, a microRNA binding site is recognized by a microRNA in hepatic cells.

In certain embodiments, an RNA of the present disclosure comprises one or more phosphonate modifications selected from a phosphorothioate linkage (PS), phosphorodithioate linkage (PS2), methylphosphonate linkage (MP), methoxypropylphosphonate linkage (MOP), 5′-(E)-vinylphosphonate linkage (5′-(E)-VP), 5′-Methyl Phosphonate linkage (5′-MP), (S)-5′-C-methyl with phosphate linkage, 5′-phosphorothioate linkage (5′-PS), and a peptide nucleic acid linkage (PNA). In certain embodiments, an RNA of the present disclosure comprises one or more ribose modifications selected from a 2′-O-methyl (2′-OMe), 2′-O-methoxyethyl (2′-O-MOE), 2′-deoxy-2′-fluoro (2′-F), 2′-arabino-fluoro (2′-Ara-F), 2′-O-benzyl, 2′-O-methyl-4-pyridine (2′-O-CH2Py (4)), Locked nucleic acid (LNA), (S)-cET-BNA, tricyclo-DNA (tcDNA), PMO, Unlocked Nucleic Acid (UNA) and glycol nucleic acid (GNA). In certain embodiments, the RNA comprises a Locked Nucleic Acid (LNA) comprising a methyl bridge, an ethyl bridge, a propyl bridge, a butyl bridge or an optionally substituted variant of any of the aforementioned. In certain embodiments, an RNA of the present disclosure comprises one or more modified bases selected from a pseudouridine (w), 2′thiouridine (s2U), N6′-methyladenosine (m6A), 5′methylcytidine (m⁵C), 5′fluoro2′-deoxyuridine, N-ethylpiperidine 7′-EAA triazole modified adenine, N-ethylpiperidine 6′triazole modified adenine, 6′pheynlpyrrolo-cytosine (PhpC), 2′, 4′-difluorotoluyl ribonucleoside (rF), and 5′-nitroindole.

### Single Stranded DNA Payloads

In various embodiments, the LNPs of the present disclosure may comprise a payload having at least one single stranded DNA. In certain embodiments, the single stranded DNA is a linear single stranded DNA. In certain embodiments, the single stranded DNA is a circular single stranded DNA. In certain embodiments, the payload further comprises a nucleobase editing system, such as an enzyme or polynucleotide encoding an enzyme capable of independently or co-dependently editing, modifying, or altering a target polynucleotide sequence or a target transcript comprising a nucleic acid sequence.

In certain embodiments, the circular single stranded DNA (CiSSD) payload is one described in PCT Publication WO2020142730A1, which is incorporated by reference herein in its entirety. In certain embodiments, the CiSSD is a donor template for use as part of a nucleobase editing system for targeted genome modification. In certain embodiments, the CiSSD comprises a DNA insert, a 5′ homology arm, and a 3′ homology arm. In some embodiments, the DNA insert is located between 5′ homology arm and 3′ homology arm. Homology arms as used herein refer to a series of nucleotides that are complementary to a series of nucleotides in an endogenous DNA sequence in the target region. The homology arms flanking the DNA insert allow for specific insertion of the DNA insert in the target region. A target region is a nucleic acid sequence where a desired insertion or modification occurs.

In certain embodiments, the DNA insert is at least 1 nucleotide. In certain embodiments, the DNA insert is at least about 0.5 kb, 2 kb, 2.5 kb, 5 kb, 10 kb, 20 kb, 40 kb, 80 kb, 100 kb, 150 kb, or 200 kb. In certain embodiments, the length of the DNA insert is about 0.5 kb to 5 kb, about 1 kb to 5 kb, about 1 kb to 10 kb, about 1.6 kb to 5 kb, about 1.6 kb to 10 kb, about 2 kb to 5 kb, about 2 kb to 20 kb, about 2.5 kb to 5 kb, about 2.5 kb to 10 kb, about 2.5 kb to 20 kb, and about 5 kb to 100 kb. In some embodiments, the DNA insert size may range from about 1 kb to about 3 kb, about 3 kb to about 6 kb, about 6 kb to about 9 kb, about 9 kb to about 12 kb, about 12 kb to about 15 kb, about 15 kb to about 18 kb, or about 18 kb to about 21 kb.

In some embodiments, the DNA insert may comprise a nucleotide sequence that encodes a maker or a reporter, e.g., a fluorescent marker, an antibiotic marker, or any suitable marker. A “marker” or “reporter” as used herein means a feature that allows for identification and selection of a desired cell, e.g., by fluorescence or antibiotic resistance. For example, the insert may include a nucleotide sequence encoding a reporter (e.g, GFP, RFP, or any suitable reporter) or a recombinase. For example, the reporter is an N-terminal GFP fusion reporter.

In some embodiments, the DNA insert may comprise a nucleotide sequence that encodes a transcription unit, wherein each transcription unit can produce a cellular product (e.g, protein or RNA). In some embodiments, the DNA insert may comprise a nucleotide sequence that encodes a protein, e.g, an immunomodulatory protein (e.g, a cytokine), an antibody, a chimeric antigen receptor (CAR), a growth factor, a T cell receptor, or another protein.

In certain embodiments, the CiSSD comprises a DNA insert that can be inserted at a nucleotide break in a target region of genomic DNA. In some embodiments, the break is a double stranded break (DSB). In certain embodiments, the break is a single stranded DNA break or a nick. Precision gene editing techniques, e.g, CRISPR, create a break near a desired sequence change (target sequence). CRISPR can be applied to produce deletions, disruptions, insertions, replacements, and repairs. The components of template donors for these different modifications is generally the same, consisting of three basic elements: a 5′ homology arm, a DNA insert, and a 3′ homology arm. CRISPR-based gene editing can generate gene knockouts by disrupting the gene sequence, however, efficiency for inserting exogenous DNA (knock-in) or replacement of genomic sequences is very poor using current methods. In certain embodiments, CiSSDs may be used with CRISPR by generating a knock-in modification. Double-stranded breaks can be introduced by any suitable mechanism, including, for example, by gene-editing systems using CRISPR, zinc finger nuclease, TALEN nuclease (Transcription Activator-Like Effector Nuclease), or meganuclease as described previously. Briefly, the CRISPR genome editing system generates a targeted DSB using the CRISPR programmable DNA endonuclease that can be targeted to a specific DNA sequence (target sequence) by a small “guide” RNA (crRNA). Guide RNAs for use in CRISPR-based modification (z.e., crRNAs and tracrRNAs) may be generated by any suitable method. In certain embodiments, crRNAs and tracrRNAs may be chemically synthesized. In other embodiments, a single guide RNA (sgRNA) may be constructed and synthesized by in vitro transcription.

In certain embodiments, an LNP of the present disclosure comprises a CiSSD disclosed herein and further comprises a precision gene editing system component such as a CRISPR, zinc finger nuclease, TALEN nuclease (Transcription Activator-Like Effector Nuclease), or meganuclease, or any other nucleobase editing system known in the art.

In certain embodiments, the single stranded DNA (SSD) payload is one described in PCT Publication WO2020232286A1, which is incorporated by reference herein in its entirety.

In certain embodiments, the SSD comprises an engineered initiator sequence and an engineered terminator sequence from a filamentous bacteriophage, and a DNA sequence of interest, wherein the DNA sequence of interest is located 3′ to the engineered initiator sequence and 5′ to the engineered terminator sequence. In certain embodiments, the SSD comprises a selectable marker.

In certain embodiments, the single stranded DNA (SSD) payload is made by a method described in PCT Publication WO2020232286A1. In certain embodiments, the SSD is made by a the method comprising: (a) culturing a host cell of claim 11 under conditions suitable for producing a ssDNA from the DNA sequence of interest in the engineered nucleic acid and the plurality of bacteriophage proteins from the nucleic acid helper plasmid; (b) allowing the ssDNA and the plurality of bacteriophage proteins to assemble into an engineered phage; and (c) collecting the engineered phage. In certain embodiments, the method further comprises extracting the SSD from the engineered phage.

In certain embodiments, at least 90% of the SSD is the same length as the DNA sequence of interest. In certain embodiments, at least 95% of the ssDNA is the same length as the DNA sequence of interest. In certain embodiments, the SSD is between 100 and 20,000 nucleotides in length. In certain embodiments, the ssDNA is circular.

In certain embodiments, the single stranded DNA (SSD) payload is one described in PCT Publication WO2022011082A1, which is incorporated by reference herein in its entirety. In certain embodiments, the SSD comprises a first sequence from a filamentous bacteriophage, the first sequence having both initiator and terminator functions; a second sequence that is identical to the first sequence; and a single-strand DNA sequence of interest that is located between the first sequence and the second sequence. In certain embodiments, the SSD further comprises a selectable marker. In certain embodiments, the SSD is circular. In certain embodiments, the SSD is linear.

In certain embodiments, the single stranded DNA (SSD) payload is made by a method described in PCT Publication WO2022011082A1. In certain embodiments, the method comprises culturing a host cell under conditions suitable for producing the single-stranded DNA from the single-strand DNA sequence of interest in the isolated nucleic acid and producing the bacteriophage proteins from the nucleic acid helper plasmid; allowing the single-stranded DNA and bacteriophage proteins to assemble into an engineered phage; and collecting the engineered phage. In certain embodiments, the host cell comprises an isolated nucleic acid that includes: a first sequence from a filamentous bacteriophage, the first sequence having both initiator and terminator functions; a second sequence that is identical to the first sequence; and a single-strand DNA sequence of interest that is located between the first sequence and the second sequence, and a nucleic acid helper plasmid for expressing bacteriophage proteins capable of assembling a single-strand DNA into a bacteriophage. In certain embodiments, the method further comprises extracting the SSD from the engineered phage.

In certain embodiments, at least 90% of the SSD is the same length as the DNA sequence of interest. In certain embodiments, at least 95% of the ssDNA is the same length as the DNA sequence of interest. In certain embodiments, the SSD is between 100 and 20,000 nucleotides in length. In certain embodiments, the SSD is circular.

In certain embodiments, the single stranded DNA (SSD) payload is one described in PCT Publication WO2021055616A1, which is incorporated by reference herein in its entirety.

Linear mRNA Payloads

In various embodiments, the compositions may comprise linear mRNA molecules.

Ribonucleic acid (RNA) is a molecule that is made up of nucleotides, which are ribose sugars attached to nitrogenous bases and phosphate groups. The nitrogenous bases include adenine (A), guanine (G), uracil (U), and cytosine (C). Generally, RNA mostly exists in the single-stranded form but can also exists double-stranded in certain circumstances. The length, form and structure of RNA is diverse depending on the purpose of the RNA. For example, the length of an RNA can vary from a short sequence (e.g., siRNA) to a long sequences (e.g., lncRNA), can be linear (e.g., mRNA) or circular (e.g., oRNA), and can either be a coding (e.g., mRNA) or a non-coding (e.g., lncRNA) sequence.

In various embodiments, the LNP-based nucleobase editing systems, RNA therapeutics and pharmaceutical compositions thereof described herein can be used to deliver a mRNA payload that is a linear mRNA molecule. In embodiments, the mRNA payload may comprise one or more nucleotide sequences that encode a product of interest, such as, but not limited to a component of a gene editing system (e.g. an endonuclease, a prime editor, etc.) and/or a therapeutic protein.

In some embodiments, the RNA payload may be a linear mRNA. As used herein, the term “messenger RNA” (mRNA) refers to any polynucleotide which encodes a protein of interest and which is capable of being translated to produce the encoded protein of interest in vitro, in vivo, in situ or ex vivo.

Generally, a mRNA molecule comprises at least a coding region, a 5′ untranslated region (UTR), a 3′ UTR, a 5′ cap and a poly-A tail. In some aspects, one or more structural and/or chemical modifications or alterations may be included in the RNA which can reduce the innate immune response of a cell in which the mRNA is introduced. As used herein, a “structural” feature or modification is one in which two or more linked nucleotides are inserted, deleted, duplicated, inverted or randomized in a nucleic acid without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”.

Generally, a coding region of interest in an mRNA used herein may encode a dipeptide, a tripeptide, a tetrapeptide, a pentapeptide, a hexapeptide, a heptapeptide, an octapeptide, a nonapeptide, or a decapeptide. In another embodiment, the mRNA may encode a peptide of 2-30 amino acids, e.g. 5-30, 10-30, 2-25, 5-25, 10-25, or 10-20 amino acids. The mRNA may encode a peptide of at least 10, 11, 12, 13, 14, 15, 17, 20, 25, 26, 27, 28, 29, 30, 31, 32, 33, 34, 35, 36, 37, 38, 39, or 40 amino acids, or a peptide that is no longer than 10, 11, 12, 13, 14, 15, 17, 20, 25, 26, 27, 28, 29, 30, 31, 32, 33, 34, 35, 36, 37, 38, 39, or 40 amino acids.

Generally, the length of the region of the mRNA encoding a product of interest is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the mRNA has a total length that spans from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

In some embodiments, the region or regions flanking the region encoding the product of interest may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

In some embodiments, the mRNA comprises a tailing sequence which can range from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

In some embodiments, the mRNA comprises a capping sequence which comprises a single cap or a series of nucleotides forming the cap. The capping sequence may be from 1 to 10, e.g. 2-9, 3-8, 4-7, 1-5, 5-10, or at least 2, or 10 or fewer nucleotides in length. In some embodiments, the caping sequence is absent.

In some embodiments, the mRNA comprises a region comprising a start codon. The region comprising the start codon may range from 3 to 40, e.g., 5-30, 10-20, 15, or at least 4, or 30 or fewer nucleotides in length.

In some embodiments, the mRNA comprises a region comprising a stop codon. The region comprising the stop codon may range from 3 to 40, e.g., 5-30, 10-20, 15, or at least 4, or 30 or fewer nucleotides in length.

In some embodiments, the mRNA comprises a region comprising a restriction sequence. The region comprising the restriction sequence may range from 3 to 40, e.g., 5-30, 10-20, 15, or at least 4, or 30 or fewer nucleotides in length.

### Untranslated Regions (UTRs)

In various embodiments, the mRNA payloads of the LNP-based nucleobase editing systems, RNA therapeutics and pharmaceutical compositions thereof described herein, may comprise at least one untranslated region (UTR) which flanks the region encoding the product of interest and/or is incorporated within the mRNA molecule. UTRs are transcribed by not translated. The mRNA payloads can include 5′ UTR sequences and 3′ UTR sequences, as well as internal UTRs.

The RNA payloads of the present disclosure may comprise one or more regions or parts which act or function as an untranslated region. Where nucleic acids are designed to encode at least one polypeptide of interest, the nucleic acid may comprise one or more of these untranslated regions (UTRs). Wild-type untranslated regions of a nucleic acid are transcribed but not translated. In mRNA, 5′ UTR starts at the transcription start site and continues to the start codon but does not include the start codon; whereas, 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the RNA payload molecules (e.g., linear and circular mRNA molecules) of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

In various embodiments, the mRNA payloads of the LNP-based nucleobase editing systems, RNA therapeutics and pharmaceutical compositions thereof described herein, may comprise at least one UTR that may be selected from any UTR sequence listed in Tables 19 or 20 of U.S. Pat. No. 10,709,779, which is incorporated herein by reference.

### 5′ UTR Regions

In various embodiments, the mRNA payloads of the LNP-based nucleobase editing systems, RNA therapeutics and pharmaceutical compositions thereof described herein, may comprise at least one 5′ UTR.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR (A/G)CCAUGG (SEQ ID NO: 401), where R is a purine (adenine or guanine) three bases upstream of the start codon (AUG), which is followed by another ‘G’. 5′UTR also have been known to form secondary structures which are involved in elongation factor binding. 5′ UTR sequences are also known to be important for ribosome recruitment to the mRNA and have been reported to play a role in translation (Hinnebusch A, et al., (2016) Science, 352:6292: 1413-6). In addition, 5′ UTR sequences may confer increased half-life, increased expression and/or increased activity of a polypeptide encoded by the RNA payload described herein.

In various embodiments, the RNA payload constructs contemplated herein may include 5′UTRs that are found in nature and those that are not. For example, 5′UTRs can be synthetic and/or can be altered in sequence with respect to a naturally occurring 5′UTR. Such altered 5′UTRs can include one or more modifications relative to a naturally occurring 5′UTR, such as, for example, an insertion, deletion, or an altered sequence, or the substitution of one or more nucleotide analogs in place of a naturally occurring nucleotide.

The 5′ UTR starts at the transcription start site and continues to the start codon but does not include the start codon; whereas, the 3 ‘UTR starts immediately following the stop codon and continues until the transcriptional termination signal. While not wishing to be bound by theory, the UTRs may have a regulatory role in terms of translation and stability of the nucleic acid.

Natural 5′ UTRs usually include features which have a role in translation initiation as they tend to include Kozak sequences which are commonly known to be involved in the process by which the ribosome initiates translation of many genes. Kozak sequences have the consensus CCR (A/G)CCAUGG, where R is a purine (adenine or guanine) three bases upstream of the start codon (AUG), which is followed by another ‘G’. 5′UTR also have been known to form secondary structures which are involved in elongation factor binding.

In an embodiment, 5′ UTR comprises a sequence provided in Table X or a sequence having at least 80%, 85%, 90%, 95%, 96%, 97%, 98%, 99% or 100% sequence identity to a 5′ UTR sequence provided in Table X, or a variant or a fragment thereof (e.g., a fragment that lacks the first one, two, three, four, five, or six nucleotides of 5′ UTR sequence provided in Table X). In an embodiment, 5′ UTR comprises a sequence with at least 80%, 85%, 90%, 95%, 96%, 97%, 98%, 99% or 100% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 29.

TABLE X
Exemplary nucleotide sequences
of 5′ UTRs

| 5′ UTR Nucleotide | Sequence |
|---|---|
| Sequence | Identifier |
| ggaaaucgca aaauuugcuc | SEQ ID |
| uucgcguuag auuucuuuua | NO: 402 |
| guuuucucgc aacuagcaag | |
| cuuuuuguuc ucgccgccgc c | |
| ggaaaucgca aaauuugcuc | SEQ ID |
| uucgcguuag auuucuuuua | NO: 403 |
| guuuucucgc aacuagcaag | |
| cuuuuuguuc ucgccgccgc c | |
| ggaaaucgca aaauuuucuu | SEQ ID |
| uucgcguuag auuucuuuua | NO: 404 |
| guuuucuuuc aacuagcaag | |
| cuuuuuguuc ucgccgccgc c | |
| ggaaaucgca aaauuuuugc | SEQ ID |
| ucuuuuucgc guuagauuuc | NO: 405 |
| uuuuaguuuu cuykcaacua | |
| gcaagcuuuu uguucucgcc rcc | |
| ggaaaucccc acaaccgccu | SEQ ID |
| cauauccagg cucaagaaua | NO: 406 |
| gagcucagug uuuuguuguu | |
| uaaucauucc gacguguuuu | |
| gcgauauucg cgcaaagcag | |
| ccagucgcgc gcuugcuuuu | |
| aaguagaguu guuuuuccac | |
| ccguuugcca ggcaucuuua | |
| auuuaacaua uuuuuauuuu | |
| ucaggcuaac cuacgccgcc acc | |
| ggaaauaaga gagaaaagaa | SEQ ID |
| gaguaagaag aaauauaaga | NO: 407 |
| ucucccugag cuucagggag | |
| ccccggcgcc gccacc | |
| ggaaaccccc cacccccgua | SEQ ID |
| agagagaaaa gaagaguaag | NO: 408 |
| aagaaauaua agaucucccu | |
| gagcuucagg gagccccggc | |
| gccgccacc | |
| ggagaacuuc cgcuuccguu | SEQ ID |
| ggcgcaagcg cuuucauuuu | NO: 409 |
| uucugcuacc gugacuaag | |
| ggaaauaaga gagaaaagaa | SEQ ID |
| gaguaagaag aaauauaaga | NO: 410 |
| gccacc | |
| ggaaauaaga gagaaaagaa | SEQ ID |
| gaguaagaag aaauauaaga | NO: 411 |
| ccccggcgcc gccacc | |
| ggaaacuuua uuuaguguua | SEQ ID |
| cuuuauuuuc uguuuauuug | NO: 412 |
| uguuucuuca guggguuugu | |
| ucuaauuucc uuggccgcc | |
| ggaaaaucug uauuagguug | SEQ ID |
| gcguguucuu uggucgguug | NO: 413 |
| uuaguauugu uguugauucg | |
| uuuguggucg guugccgcc | |
| ggaaaauuau uaacaucuug | SEQ ID |
| guauucucga uaaccauucg | NO: 414 |
| uuggauuuua uuguauucgu | |
| aguuuggguu ccugcgcc | |
| ggaaauuauu auuauuucua | SEQ ID |
| gcuacaauuu aucauuguau | NO: 415 |
| uauuuuagcu auucaucauu | |
| auuuacuugg ugaucaaca | |
| ggaaauaggu uguuaaccaa | SEQ ID |
| guucaagccu aauaagcuug | NO: 416 |
| gauucuggug acuugcuuca | |
| ccguuggcgg gcaccgauc | |
| ggaaaucgua gagagucgua | SEQ ID |
| cuuaguacau aucgacuauc | NO: 417 |
| gguggacacc aucaagauua | |
| uaaaccaggc caga | |
| ggaaacccgc ccaagcgacc | SEQ ID |
| ccaacauauc agcaguugcc | NO: 418 |
| caaucccaac ucccaacaca | |
| auccccaagc aacgccgcc | |
| ggaaagcgau ugaaggcguc | SEQ ID |
| uuuucaacua cucgauuaag | NO: 419 |
| guuggguauc gucgugggac | |
| uuggaaauuu guuguuucc | |
| ggaaacuaau cgaaauaaaa | SEQ ID |
| gagccccgua cucuuuuauu | NO: 420 |
| ucuauuaggu uaggagccuu | |
| agcauuugua ucuuaggua | |
| ggaaauguga uuuccagcaa | SEQ ID |
| cuucuuuuga auauauugaa | NO: 421 |
| uuccuaauuc aaagcgaaca | |
| aaucuacaag ccauauacc | |
| ggaaaucgua gagagucgua | SEQ ID |
| cuuacguggu cgccauugca | NO: 422 |
| uagcgcgcga aagcaacagg | |
| aacaagaacg cgcc | |
| ggaaaucgua gagagucgua | SEQ ID |
| cuuagaauaa acagagucgg | NO: 423 |
| gucgacuugu cucugauacu | |
| acgacgucac aauc | |
| ggaaaauuug ccuucggagu | SEQ ID |
| ugcguauccu gaacugccca | NO: 424 |
| gccuccugau auacaacugu | |
| uccgcuuauu cgggccgcc | |
| ggaaaucuga gcaggaaucc | SEQ ID |
| uuugugcauu gaagacuuua | NO: 425 |
| gauuccucuc ugcgguagac | |
| gugcacuuau aaguauuug | |
| ggaaagcgau ugaaggcguc | SEQ ID |
| uuuucaacua cucgauuaag | NO: 426 |
| guuggguauc gucgugggac | |
| uuggaaauuu guugccacc | |
| ggaaaauuuu agccuggaac | SEQ ID |
| guuagauaac uguccuguug | NO: 427 |
| ucuuuauaua cuuggucccc | |
| aaguaguuug ucuuccaaa | |
| ggaaauuuuu uuuugauauu | SEQ ID |
| auaagaguuu uuuuuugaua | NO: 428 |
| uuaagaaaau uuuuuuuuga | |
| uauuagaaga guaagaagaa | |
| auauaagacc ccggcgccgc cacc | |
| ggaaauaaga gagaaaagaa | SEQ ID |
| gaguaagaag aaauauaaga | NO: 429 |
| gccaaaaaaa aaaaacc | |
| ggaaaucucc cugagcuuca | SEQ ID |
| gggaguaaga gagaaaagaa | NO: 430 |
| gaguaagaag aaauauaaga | |
| ccccggcgcc gccacc | |

In some embodiments of the disclosure, a 5′ UTR is a heterologous UTR, i.e., is a UTR found in nature associated with a different mRNA. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived alpha-globin or beta-globin (e.g., U.S. Pat. Nos. 8,278,063 and 9,012,219), human cytochrome b-245 polypeptide, and hydroxysteroid (17b) dehydrogenase, and Tobacco etch virus. CMV immediate-early 1 (IE1) gene (see US20140206753 and WO2013/185069), the sequence GGGAUCCUACC(SEQ ID NO: 21)(WO2014144196) may also be used. In another embodiment, 5′ UTR of a TOP gene is a 5′ UTR of a TOP gene lacking 5′ TOP motif (the oligopyrimidine tract)(e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738)), 5′ UTR element derived from the 5′UTR of an hydroxysteroid (17-β) dehydrogenase 4 gene (HSD17B4) (WO2015024667), or a 5′ UTR element derived from the 5′ UTR of ATP5A1 (WO2015024667) can be used. In one embodiment, an internal ribosome entry site (IRES) is used as a substitute for a 5′ UTR.

In some embodiments, a 5′ UTR of the present disclosure comprises a sequence selected from SEQ ID NO: 431 (GGGAAAUAAG AGAGAAAAGA AGAGUAAGAA GAAAUAUAAG AGCCACC), SEQ ID NO:432 (GGGAAATAAG AGAGAAAAGA AGAGTAAGAA GAAATATAAG AGCCACC), SEQ ID NO:433 (GGGAAAUAAG AGAGAAAAGA AGAGUAAGAA GAAAUAUAAG AGCCACC) and SEQ ID NO:434 (GGGAAATAAG AGAGAAAAGA AGAGTAAGAA GAAATATAAG AGCCACC).

### 3′ UTR Regions

In various embodiments, the mRNA payloads of the LNP-based nucleobase editing systems, RNA therapeutics and pharmaceutical compositions thereof described herein, may comprise at least one 3′ UTR. 3′ UTRs may be heterologous or synthetic.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA (U/A)(U/A)(SEQ ID NO: 35) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into 3′ UTR of nucleic acid molecules will lead to HuR binding and thus, stabilization of the message in vivo.

3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al., 1995): Class I AREs contain several dispersed copies of an AUUUA motif within U-rich regions. C-Myc and MyoD contain class I AREs. Class II AREs possess two or more overlapping UUAUUUA (U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into 3′ UTR of nucleic acid molecules will lead to HuR binding and thus, stabilization of the message in vivo.

Introduction, removal or modification of 3′ UTR AU rich elements (AREs) can be used to modulate the stability of the mRNA payloads described herein. For example, one or more copies of an ARE can be introduced to make mRNA less stable and thereby curtail translation and decrease production of the resultant protein. Alternatively, AREs can be identified and removed or mutated to increase the intracellular stability and thus increase translation and production of the resultant protein.

In some embodiments, the introduction of features often expressed in genes of target organs the stability and protein production of the mRNA can be enhanced in a specific organ and/or tissue. As a non-limiting example, the feature can be a UTR. As another example, the feature can be introns or portions of introns sequences.

Those of ordinary skill in the art will understand that 5′ UTRs that are heterologous or synthetic may be used with any desired 3′ UTR sequence. For example, a heterologous 5′ UTR may be used with a synthetic 3′ UTR with a heterologous 3′ UTR.

Non-UTR sequences may also be used as regions or subregions within an RNA payload construct. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the polypeptide coding region of interest in an mRNA payload may be flanked by a 5′ UTR which may contain a strong Kozak translational initiation signal and/or a 3′ UTR which may include an oligo (dT) sequence for templated addition of a poly-A tail. 5′ UTR may comprise a first polynucleotide fragment and a second polynucleotide fragment from the same and/or different genes such as 5′ UTRs described in US Patent Application Publication No. 20100293625 and PCT/US2014/069155, herein incorporated by reference in its entirety

It should be understood that any UTR from any gene may be incorporated into the regions of an RNA payload molecule (e.g., a linear mRNA). Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in U.S. application Ser. No. 20/090,226470, herein incorporated by reference in its entirety, and those known in the art.

### 5′ Capping

In various embodiments, the mRNA payloads of the LNP-based nucleobase editing systems, RNA therapeutics and pharmaceutical compositions thereof described herein, may comprise a 5′ cap structure.

The 5′ cap structure of an mRNA is involved in nuclear export, increasing mRNA stability and binds the mRNA Cap Binding Protein (CBP), which is responsible for mRNA stability in the cell and translation competency through the association of CBP with poly (A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to mRNA may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in 5′-ppp-5′ cap.

Additional modified guanosine nucleotides may be used such as α-methylphosphonate and seleno-phosphate nucleotides.

Additional modifications include, but are not limited to, 2′-O-methylation of the ribose sugars of 5 ‘-terminal and/or 5′-anteterminal nucleotides of the mRNA (as mentioned above) on the 2′-hydroxyl group of the sugar ring. Multiple distinct 5 ‘-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5 ′-guanosine (m7G-3′mppp-G; which may equivalently be designated 3′ O-Me-m7G (5′) ppp (5′) G). The 3′-0 atom of the other, unmodified, guanine becomes linked to 5′-terminal nucleotide of the capped nucleic acid molecule (e.g. an mRNA). The N7- and 3′-O-methlyated guanine provides the terminal moiety of the capped nucleic acid molecule (e.g. mRNA).

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5 ′-cap structures of nucleic acids produced by the endogenous, cellular transcription machinery, may lead to reduced translational competency and reduced cellular stability.

mRNA may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5 ′cap structures are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5 ′cap structure). For example, recombinant Vaccinia Virus Capping Enzyme and recombinant 2′-O-methyltransferase enzyme can create a canonical 5 ′-5′-triphosphate linkage between the 5 ′-terminal nucleotide of an mRNA and a guanine cap nucleotide wherein the cap guanine contains an N7 methylation and the 5′-terminal nucleotide of the mRNA contains a 2′-O-methyl. Such a structure is termed the Capl structure. This cap results in a higher translational-competency and cellular stability and a reduced activation of cellular pro-inflammatory cytokines, as compared, e.g., to other 5 ′cap analog structures known in the art. Cap structures include, but are not limited to, 7 mG (5*) ppp (5*)N,pN2p (cap 0), 7 mG (5*) ppp (5*)NlmpNp (cap 1), and 7 mG (5*)-ppp (5′)NlmpN2mp (cap 2).

In some embodiments, 5′ terminal caps may include endogenous caps or cap analogs.

In some embodiments, a 5′ terminal cap may comprise a guanine analog. Useful guanine analogs include, but are not limited to, inosine, Nl-methylguanosine, 2′fluoro-guanosine, 7-deaza-guanosine, 8-oxo-guanosine, 2-amino-guanosine, LNA-guanosine, and 2-azido-guanosine.

### IRES Sequences

In various embodiments, the mRNA payloads of the LNP-based nucleobase editing systems, RNA therapeutics and pharmaceutical compositions thereof described herein, may comprise one or more IRES sequences.

In some embodiments, the mRNA may contain an internal ribosome entry site (IRES). First identified as a feature Picorna virus RNA, IRES plays an important role in initiating protein synthesis in absence of 5′ cap structure. An IRES may act as the sole ribosome binding site, or may serve as one of multiple ribosome binding sites of an mRNA. An mRNA that contains more than one functional ribosome binding site may encode several peptides or polypeptides that are translated independently by the ribosomes. Non-limiting examples of IRES sequences that can be used include without limitation, those from picornaviruses (e.g. FMDV), pest viruses (CFFV), polio viruses (PV), encephalomyocarditis viruses (ECMV), foot- and -mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

In some embodiments, the IRES is from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectrop is obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human ATIR, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BNS, Salivirus A BN2, Salivirus A 02394, Salivirus A GUT, Salivirus A CH, Salivirus A SZ1, Salivirus FHB, CVB3, CVB1, Echovirus 7, CVBS, EVA71, CVA3, CVA12, EV24 or an aptamer to eIF4G.

### Poly-A Tails and 3′ Stabilizing Region

In various embodiments, the mRNA payloads of the LNP-based nucleobase editing systems, RNA therapeutics and pharmaceutical compositions thereof described herein, may comprise a poly-A tail.

During RNA processing, a long chain of adenine nucleotides (poly-A tail) may be added to a polynucleotide such as an mRNA molecules in order to increase stability. Immediately after transcription, 3′ end of the transcript may be cleaved to free a 3′ hydroxyl. Then poly-A polymerase adds a chain of adenine nucleotides to the free 3′ hydroxyl end. The process, called polyadenylation, adds a poly-A tail of a certain length. In some embodiments, the length of a poly-A tail is greater than 30

nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides) and no more than about 50, 100, 200, 300, 400, 500, 600, 700, 800, 900, 1000, 2000, or 3000 nucleotides in length. In some embodiments, the mRNA includes a poly-A tail from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall mRNA. This design may be based on the length of the region coding for a target of interest, the length of a particular feature or region (such as a flanking region), or based on the length of the ultimate product expressed from the mRNA.

In this context the poly-A tail may be 10, 20, 30, 40, 50, 60, 70, 80, 90, or 100% greater in length than the mRNA or feature thereof. The poly-A tail may also be designed as a fraction of mRNA to which it belongs. In this context, the poly-A tail may be 10, 20, 30, 40, 50, 60, 70, 80, or 90% or more of the total length of the construct or the total length of the construct minus the poly-A tail. Further, engineered binding sites and conjugation of mRNA for poly-A binding protein may enhance expression.

Additionally, multiple distinct mRNA may be linked together to the PABP (Poly-A binding protein) through 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the mRNA are designed to include a polyA-G Quartet. The G-quartet is a cyclic hydrogen bonded array of four guanine nucleotides that can be formed by G-rich sequences in both DNA and RNA. In this embodiment, the G-quartet is incorporated at the end of the poly-A tail.

### Stop Codons

In various embodiments, the mRNA payloads of the LNP-based nucleobase editing systems, RNA therapeutics and pharmaceutical compositions thereof described herein, may comprise one or more translation stop codons. Translational stop codons, UAA, UAG, and UGA, are an important component of the genetic code and signal the termination of translation of an mRNA. During protein synthesis, stop codons interact with protein release factors and this interaction can modulate ribosomal activity thus having an impact translation (Tate W P, et al., (2018) Biochem Soc Trans, 46 (6): 1615-162).

A stop element as used herein, refers to a nucleic acid sequence comprising a stop codon. The stop codon can be selected from TGA, TAA and TAG in the case of DNA, or from UGA, UAA and UAG in the case of RNA. In an embodiment, a stop element comprises two consecutive stop codons. In an embodiment, a stop element comprises three consecutive stop codons. In an embodiment, a stop element comprises four consecutive stop codons. In an embodiment, a stop element comprises five consecutive stop codons.

In some embodiments, the mRNA may include one stop codon. In some embodiments, the mRNA may include two stop codons. In some embodiments, the mRNA may include three stop codons. In some embodiments, the mRNA may include at least one stop codon. In some embodiments, the mRNA may include at least two stop codons. In some embodiments, the mRNA may include at least three stop codons. As non-limiting examples, the stop codon may be selected from TGA, TAA and TAG.

In other embodiments, the stop codon may be selected from one or more of the following stop elements of Table Y:

TABLE Y
Additional stop elements

| Nucleotide | |
|---|---|
| sequence | Sequence |
| (5′ to 3′) | Identifier |
| UGAUAAUAG | SEQ ID NO: 435 |
| UAAUAGUAA | SEQ ID NO: 436 |
| UAAGUCUAA | SEQ ID NO: 437 |
| UAAAGCUAA | SEQ ID NO: 438 |
| UAAGUCUCC | SEQ ID NO: 439 |
| UAAGGCUAA | SEQ ID NO: 440 |
| UAAGCCCCUCCGGGG | SEQ ID NO: 441 |
| UAAAGCUCCCCGGGG | SEQ ID NO: 442 |
| UAAGCCCCU | SEQ ID NO: 443 |
| UAAAGCUCC | SEQ ID NO: 444 |
| UAAAGCUCC | SEQ ID NO: 445 |
| UAGGGUUAA | SEQ ID NO: 446 |
| UAAGCACCC | SEQ ID NO: 447 |
| UGAUAGUAA | SEQ ID NO: 448 |
| UAAAGCGCU | SEQ ID NO: 449 |

In some embodiments, the mRNA includes the stop codon TGA and one additional stop codon. In a further embodiment the addition stop codon may be TAA.

### MicroRNA Binding Sites and Other Regulatory Elements

In various embodiments, the mRNA payloads of the LNP-based nucleobase editing systems, RNA therapeutics and pharmaceutical compositions thereof described herein, may comprise one or more regulatory elements, including, but not limited to microRNA (miRNA) binding sites, structured mRNA sequences and/or motifs, artificial binding sites to bind to endogenous nucleic acid binding molecules, and combinations thereof.

### Chemically Unmodified Nucleotides

In some embodiments, the mRNA payloads of the LNP-based nucleobase editing systems, RNA therapeutics and pharmaceutical compositions thereof described herein are not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemically Modified Nucleotides

In some embodiments, the mRNA payloads of the LNP-based nucleobase editing systems, RNA therapeutics and pharmaceutical compositions thereof described herein comprise, in some embodiments, comprises at least one chemical modification.

The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” of they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (w), N1-methylpseudouridine (m1w), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of 1-methyl-pseudouridine (m1w), 5-methoxy-uridine (mo5U), 5-methyl-cytidine (m⁵C), pseudouridine (w), a-thio-guanosine and a-thio-adenosine. In some embodiments, polynucleotides includes a combination of at least two (e.g., 2, 3, 4 or more) of the aforementioned modified nucleobases.

In some embodiments, polynucleotides (e.g., RNA polynucleotides, such as mRNA polynucleotides) comprise pseudouridine (w) and 5-methyl-cytidine (m⁵C). In some embodiments, polynucleotides (e.g., RNA polynucleotides, such as mRNA polynucleotides) comprise 1-methyl-pseudouridine (mlw). In some embodiments, polynucleotides (e.g., RNA polynucleotides, such as mRNA polynucleotides) comprise 1-methyl-pseudouridine (mlw) and 5-methyl-cytidine (m⁵C). In some embodiments, polynucleotides (e.g., RNA polynucleotides, such as mRNA polynucleotides) comprise 2-thiouridine (s2U). In some embodiments, polynucleotides (e.g., RNA polynucleotides, such as mRNA polynucleotides) comprise 2-thiouridine and 5-methyl-cytidine (m⁵C). In some embodiments, polynucleotides (e.g., RNA polynucleotides, such as mRNA polynucleotides) comprise methoxy-uridine (mo5U). In some embodiments, polynucleotides (e.g., RNA polynucleotides, such as mRNA polynucleotides) comprise 5-methoxy-uridine (mo5U) and 5-methyl-cytidine (m⁵C). In some embodiments, polynucleotides (e.g., RNA polynucleotides, such as mRNA polynucleotides) comprise 2′-O-methyl uridine. In some embodiments polynucleotides (e.g., RNA polynucleotides, such as mRNA polynucleotides) comprise 2′-O-methyl uridine and 5-methyl-cytidine (m⁵C). In some embodiments, polynucleotides (e.g., RNA polynucleotides, such as mRNA polynucleotides) comprise N6-methyl-adenosine (m6A). In some embodiments, polynucleotides (e.g., RNA polynucleotides, such as mRNA polynucleotides) comprise N6-methyl-adenosine (m6A) and 5-methyl-cytidine (mC).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m⁵C), meaning that all cytosine residues in the mRNA sequence are replaced with 5-methyl-cytidine (m⁵C). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and In some embodiments, a modified nucleobase is a modified cytosine. nucleosides having a modified uridine include 5-cyano uridine, and 4′-thio uridine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the invention, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+CorA+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

Circular mRNA Payloads

In various embodiments, the LNP-based nucleobase editing systems, RNA therapeutics and pharmaceutical compositions thereof described herein can be used to deliver an RNA payload that is a circular mRNA molecule or “ORNA.” The circular mRNA molecule may encode a CROI, such as a nucleobase editing system, or therapeutic protein as described in this specification.

In some embodiments, the RNA payload is a circular RNA (ORNA). As used herein, the terms “ORNA” or “circular RNA” are used interchangeably and can refer to a RNA that forms a circular structure through covalent or non-covalent bonds.

Circular RNA described herein are polyribonucleotides that form a continuous structure through covalent or non-covalent bonds. Due to the circular structure, oRNAs have improved stability, increased half-life, reduced immunogenicity, and/or improved functionality (e.g., of a function described herein) compared to a corresponding linear RNA.

In some embodiments, an oRNA binds a target. In some embodiments, an oRNA binds a substrate. In some embodiments, an oRNA binds a target and binds a substrate of the target. In some embodiments, an oRNA binds a target and mediates modulation of a substrate of the target. In some embodiments, an oRNA brings together a target and its substrate to mediate modification of the substrate, e.g., post-translational modification. In some embodiments, an oRNA brings together a target and its substrate to mediate a cellular process (e.g., alters protein degradation or signal transduction) involving the substrate. In some embodiments, a target is a target protein and a substrate is a substrate protein.

In some embodiments, an oRNA comprises a conjugation moiety for binding to chemical compound. The conjugation moiety can be a modified polyribonucleotide. The chemical compound can be conjugated to the oRNA by the conjugation moiety. In some embodiments, the chemical compound binds to a target and mediates modulation of a substrate of the target. In some embodiments, an oRNA binds a substrate of a target and a chemical compound conjugated to the oRNA by the conjugation moiety binds the target to bring together the target and its substrate to mediate modification of the substrate, e.g., post-translational modification. In some embodiments, an oRNA binds a substrate of a target and a chemical compound conjugated to the oRNA by the conjugation moiety binds the target to bring together the target and its substrate to mediate modification of the substrate to mediate a cellular process (e.g., alters protein degradation or signal transduction) involving the substrate. In some embodiments, a target is a target protein and a substrate is a substrate protein.

In some embodiments, the oRNA may be non-immunogenic in a mammal (e.g., a human, non-human primate, rabbit, rat, and mouse).

In some embodiments, the oRNA may be capable of replicating or replicates in a cell from an aquaculture animal (e.g., fish, crabs, shrimp, oysters etc.), a mammalian cell, a cell from a pet or zoo animal (e.g., cats, dogs, lizards, birds, lions, tigers and bears etc.), a cell from a farm or working animal (e.g., horses, cows, pigs, chickens etc.), a human cell, cultured cells, primary cells or cell lines, stem cells, progenitor cells, differentiated cells, germ cells, cancer cells (e.g., tumorigenic, metastatic), non-tumorigenic cells (e.g., normal cells), fetal cells, embryonic cells, adult cells, mitotic cells, non-mitotic cells, or any combination thereof.

In one aspect, provided herein is a pharmaceutical composition comprising: a circular RNA comprising, in the following order, a 3′ group I intron fragment, an Internal Ribosome Entry Site (IRES), an expression sequence encoding a polypeptide (e.g., a nucleobase editing system, therapeutic protein, such as a chimeric antigen receptor (CAR) or T cell receptor (TCR) complex protein), and a 5′ group I intron fragment, and a transfer vehicle comprising at least one of (i) an ionizable lipid, (ii) a structural lipid, and (iii) a PEG-modified lipid, wherein the transfer vehicle is capable of delivering the circular RNA polynucleotide to a cell (e.g., a human cell, such as an immune cell present in a human subject), such that the polypeptide is translated in the cell.

In some embodiments, the pharmaceutical composition is formulated for intravenous administration to the human subject in need thereof. In some embodiments, the 3′ group I intron fragment and 5′ group I intron fragment are Anabaena group I intron fragments.

In certain embodiments, 3′ intron fragment and 5′ intron fragment are defined by the L⁹a-5 permutation site in the intact intron. In certain embodiments, 3′ intron fragment and 5′ intron fragment are defined by the L8-2 permutation site in the intact intron.

In some embodiments, the IRES is from Taura syndrome virus, Tiiatoma virus, Theiler's encephalomyelitis virus, Simian Virus 40, Solenopsis invicta virus 1, Rhopalosiphum padi virus, Reticuloendotheliosis virus, Human poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectrop is obliqua picoma-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human ATIR, Human BAG-1, Human BCL2, Human BiP, Human c-IAPI, Human c-myc, Human eIF4G, Mouse NDST4L, Human LEF1, Mouse HIFI alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA 16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistravirus, Hubei Picoma-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24 or an aptamer to eIF4G.

In some embodiments, the IRES comprises a CVB3 IRES or a fragment or variant thereof. In some embodiments, the pharmaceutical composition comprises a first internal spacer between 3′ group I intron fragment and the IRES, and a second internal spacer between the expression sequence and 5′ group I intron fragment. In certain embodiments, the first and second internal spacers each have a length of about 10 to about 60 nucleotides.

In some embodiments, the circular mRNA comprises a nucleotide sequence encoding a polypeptide of interest, such as a nucleobase editing system or therapeutic protein (e.g., a CAR or TCR complex protein).

In embodiments where the therapeutic protein encoded by the herein RNA payload (e.g., circular or linear mRNA) is a CAR or TCR complex protein, the CAR or TCR complex protein comprises an antigen binding domain specific for an antigen selected from the group: CD 19, CD123, CD22, CD30, CD171, CS-1, C-type lectin-like molecule-1, CD33, epidermal growth factor receptor variant III (EGFRvIII), disialoganglioside GD2, disaloganglioside GD3, TNF receptor family member, B cell maturation antigen (BCMA), Tn antigen ((Tn Ag) or (GalNAca-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD 117), Interleukin-13 receptor subunit alpha-2, mesothelin, Interleukin 11 receptor alpha (IL-1 1R^{a}), prostate stem cell antigen (PSCA), Protease Serine 21, vascular endothelial growth factor receptor 2 (VEGFR2), Lewis (Y) antigen, CD24, Platelet-derived growth factor receptor beta (PDGFR-beta), Stage-specific embryonic antigen-4 (SSEA-4), CD20, Folate receptor alpha, HER2, HER3, Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gplO0), oncogene fusion protein consisting of breakpoint cluster region (BCR) and Abelson murine leukemia viral oncogene homolog 1 (Abl)(bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (0AcGD2), Folate receptor beta, tumor endothelial marker 1 (TEM1/CD248), tumor endothelial marker 7-related (TEM7R), claudin 6 (CLDN6), claudin 18.2 (CLDN18.2), thyroid stimulating hormone receptor (TSHR), G protein-coupled receptor class C group 5, member D (GPRC5D), chromosome X open reading frame 61 (CXORF61), CD97, and CD179a.

In further embodiments where the therapeutic protein encoded by the herein RNA payload (e.g., circular or linear mRNA) is a CAR or TCR complex protein, the CAR or TCR complex protein comprises a CAR comprising an antigen binding domain specific for CD19. In some embodiments, the CAR or TCR complex protein comprises a CAR comprising a costimulatory domain selected from the group CD28, 4-1BB, 0X⁴⁰, CD27, CD30, ICOS, GITR, CD40, CD2, SLAM, and combinations thereof. In some embodiments, the CAR or TCR complex protein comprises a CAR comprising a CD3zeta signaling domain. In some embodiments, the CAR or TCR complex protein comprises a CAR comprising a CH₂CH₃, CD28, and/or CD8 spacer domain. In some embodiments, the CAR or TCR complex protein comprises a CAR comprising a CD28 or CD8 transmembrane domain.

In some embodiments, the CAR or TCR complex protein comprises a CAR comprising: an antigen binding domain, a spacer domain, a transmembrane domain, a costimulatory domain, and an intracellular T cell signaling domain.

In some embodiments, the CAR or TCR complex protein comprises a multispecific CAR comprising antigen binding domains for at least two different antigens. In some embodiments, the CAR or TCR complex protein comprises a TCR complex protein selected from the group TCRalpha, TCRbeta, TCRgamma, and TCRdelta.

In some embodiments, the LNP-based nucleobase editing systems, RNA therapeutics and pharmaceutical compositions described herein further comprise a targeting moiety. In certain embodiments, the targeting moiety mediates receptor-mediated endocytosis or direct fusion of the delivery vehicle (LNPs) into selected cells of a selected cell population or tissue in the absence of cell isolation or purification. In certain embodiments, the targeting moiety is capable of binding to a protein selected from the group CD3, CD4, CD8, CDS, CD7, PD-1, 4-1BB, CD28, Clq, and CD2. In certain embodiments, the targeting moiety comprises an antibody specific for a macrophage, dendritic cell, NK cell, NKT, or T cell antigen. In certain embodiments, the targeting moiety comprises a scFv, nanobody, peptide, minibody, polynucleotide aptamer, heavy chain variable region, light chain variable region or fragment thereof.

In some embodiments, the LNP-based nucleobase editing systems, RNA therapeutics and pharmaceutical compositions described herein are administered in an amount effective to treat a disease in the human subject (e.g., wherein the disease can be cancer, muscle disorder, or CNS disorder, etc.). In some embodiments, the LNP-based nucleobase editing systems, RNA therapeutics and pharmaceutical compositions have an enhanced safety profile when compared to a pharmaceutical composition comprising T cells or vectors comprising exogenous DNA encoding the same polypeptide, e.g., a CAR complex protein.

In some embodiments, the LNP-based nucleobase editing systems and pharmaceutical compositions thereof are administered in an amount effective to mount an immunogenic response in a human subject for the vaccination against an infectious agent and/or cancer. In some embodiments, the LNP-based nucleobase editing systems and pharmaceutical compositions have an enhanced safety profile when compared to state of the art gene editing delivery compositions.

In another aspect, the present disclosure provides a circular RNA comprising, in the following order, a 3′ group I intron fragment, an Internal Ribosome Entry Site (IRES), an expression sequence encoding a polypeptide (e.g., a nucleobase editing system, therapeutic protein, such as a chimeric antigen receptor (CAR) or T cell receptor (TCR) complex protein), and a 5′ group I intron fragment.

In some embodiments, 3′ group I intron fragment and 5′ group I intron fragment are Anabaena group I intron fragments. In certain embodiments, 3′ intron fragment and 5′ intron fragment are defined by the L⁹a-5 permutation site in the intact intron. In certain embodiments, 3′ intron fragment and 5′ intron fragment are defined by the L8-2 permutation site in the intact intron. In certain embodiments, the IRES comprises a CVB3 IRES or a fragment or variant thereof.

In some embodiments, the circular RNA comprises a first internal spacer between 3′ group I intron fragment and the IRES, and a second internal spacer between the expression sequence and 5′ group I intron fragment.

In certain embodiments, the first and second internal spacers each have a length of about 10 to about 60 nucleotides.

In some embodiments, the circular RNA payload of the LNP-based nucleobase editing systems, RNA therapeutics and pharmaceutical compositions described herein consists of natural nucleotides. In some embodiments, the circular RNA further comprises a second expression sequence encoding a therapeutic protein. In some embodiments, the therapeutic protein comprises a checkpoint inhibitor. In certain embodiments, the therapeutic protein comprises a cytokine.

In some embodiments, the circular RNA payload of the LNP-based nucleobase editing systems, RNA therapeutics and pharmaceutical compositions described herein consists of natural nucleotides.

In some embodiments, the circular RNA payload LNP-based nucleobase editing systems, RNA therapeutics and pharmaceutical compositions described herein comprises a nucleotide sequence that is codon optimized, either partially or fully. In some embodiments, the circular RNA is optimized to lack at least one microRNA binding site present in an equivalent pre-optimized polynucleotide. In some embodiments, the circular RNA is optimized to lack at least one endonuclease susceptible site present in an equivalent pre-optimized polynucleotide. In some embodiments, the circular RNA is optimized to lack at least one RNA-editing susceptible site present in an equivalent pre-optimized polynucleotide.

In some embodiments, the circular RNA payload of the LNP-based nucleobase editing systems, RNA therapeutics and pharmaceutical compositions described herein has an in vivo functional half-life in humans greater than that of an equivalent linear RNA having the same expression sequence. In some embodiments, the circular RNA has a length of about 100 nucleotides to about 10 kilobases. In some embodiments, the circular RNA has a functional half-life of at least about 20 hours. In some embodiments, the circular RNA has a duration of therapeutic effect in a human cell of at least about 20 hours. In some embodiments, the circular RNA has a duration of therapeutic effect in a human cell greater than or equal to that of an equivalent linear RNA comprising the same expression sequence. In some embodiments, the circular RNA has a functional half-life in a human cell greater than or equal to that of an equivalent linear RNA comprising the same expression sequence. In some embodiments, the circular RNA payload of the LNP-based

nucleobase editing systems, RNA therapeutics and pharmaceutical compositions described herein has a half-life of at least that of a linear counterpart. In some embodiments, the ORNA has a half-life that is increased over that of a linear counterpart. In some embodiments, the half-life is increased by about 5%, 10%, 15%, 20%, 25%, 30%, 35%, 40%, 45%, 50%, or greater. In some embodiments, the oRNA has a half-life or persistence in a cell for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In some embodiments, the oRNA has a half-life or persistence in a cell for no more than about 10 mins to about 7 days, or no more than about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 24 hours (1 day), 36 hours (1.5 days), 48 hours (2 days), 60 hours (2.5 days), 72 hours (3 days), 4 days, 5 days, 6 days, or 7 days.

In some embodiments, the circular RNA payload of the LNP-based nucleobase editing systems, RNA therapeutics and pharmaceutical compositions described herein has a half-life or persistence in a cell while the cell is dividing. In some embodiments, the oRNA has a half-life or persistence in a cell post division.

In certain embodiments, the circular RNA payload of the LNP-based nucleobase editing systems, RNA therapeutics and pharmaceutical compositions described herein has a half-life or persistence in a dividing cell for greater than about 10 minutes to about 30 days, or at least about 10 minutes, 15 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In some embodiments, the circular RNA payload of the LNP-based nucleobase editing systems, RNA therapeutics and pharmaceutical compositions described herein modulates a cellular function, e.g., transiently or long term. In certain embodiments, the cellular function is stably altered, such as a modulation that persists for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer. In certain embodiments, the cellular function is transiently altered, e.g., such as a modulation that persists for no more than about 30 mins to about 7 days, or no more than about 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours (1 day), 36 hours (1.5 days), 48 hours (2 days), 60 hours (2.5 days), 72 hours (3 days), 4 days, 5 days, 6 days, or 7 days.

In some embodiments, the circular RNA payload of the LNP-based nucleobase editing systems, RNA therapeutics and pharmaceutical compositions described herein is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the oRNA may be of a sufficient size to accommodate a binding site for a ribosome.

In some embodiments, the maximum size of the circular RNA payload of the LNP-based nucleobase editing systems, RNA therapeutics and pharmaceutical compositions described herein may be limited by the ability of packaging and delivering the RNA to a target. In some embodiments, the size of the oRNA is a length sufficient to encode polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides may be useful.

In some embodiments, the circular RNA payload of the LNP-based nucleobase editing systems, RNA therapeutics and pharmaceutical compositions described herein comprises one or more elements described elsewhere herein. In some embodiments, the elements may be separated from one another by a spacer sequence or linker. In some embodiments, the elements may be separated from one another by 1 nucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides.

In some embodiments, one or more elements are contiguous with one another, e.g., lacking a spacer element.

In some embodiments, one or more elements is conformationally flexible. In some embodiments, the conformational flexibility is due to the sequence being substantially free of a secondary structure.

In some embodiments, the circular RNA payload of the LNP-based nucleobase editing systems, RNA therapeutics and pharmaceutical compositions described herein comprises a secondary or tertiary structure that accommodates a binding site for a ribosome, translation, or rolling circle translation.

In some embodiments, the circular RNA payload of the LNP-based nucleobase editing systems, RNA therapeutics and pharmaceutical compositions described herein comprises particular sequence characteristics. For example, the oRNA may comprise a particular nucleotide composition. In some such embodiments, the oRNA may include one or more purine rich regions (adenine or guanosine). In some such embodiments, the oRNA may include one or more purine rich regions (adenine or guanosine). In some embodiments, the ORNA may include one or more AU rich regions or elements (AREs). In some embodiments, the oRNA may include one or more adenine rich regions.

In some embodiments, the circular RNA payload of the LNP-based nucleobase editing systems, RNA therapeutics and pharmaceutical compositions described herein comprises one or more modifications described elsewhere herein.

In some embodiments, the circular RNA payload of the LNP-based nucleobase editing systems, RNA therapeutics and pharmaceutical compositions described herein comprises one or more expression sequences and is configured for persistent expression in a cell of a subject in vivo. In some embodiments, the oRNA is configured such that expression of the one or more expression sequences in the cell at a later time point is equal to or higher than an earlier time point. In such embodiments, the expression of the one or more expression sequences can be either maintained at a relatively stable level or can increase over time. The expression of the expression sequences can be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

### Regulatory Elements

In some embodiments, the circular RNA payload of the LNP-based nucleobase editing systems, RNA therapeutics and pharmaceutical compositions described herein comprises one or more regulatory elements. As used herein, a “regulatory element” is a sequence that modifies expression of an expression sequence, e.g., a nucleotide sequence encoding a nucleobase editing system or a therapeutic protein, i.e., a coding region of interest (CROI). The regulatory element may include a sequence that is located adjacent to a coding region of interest encoded on the circular RNA payload. The regulatory element may be operatively linked to a nucleotide sequence of the circular RNA that encodes a coding region of interest (e.g., a nucleobase editing system or therapeutic polypeptide).

In some embodiments, a regulatory element may increase an amount of expression of a coding region of interest encoded on the circular RNA payload as compared to an amount expressed when no regulatory element exists.

In some embodiments, a regulatory element may comprise a sequence to selectively initiates or activates translation of a coding sequence of interest encoded on the circular RNA payload.

In some embodiments, a regulatory element may comprise a sequence to initiate degradation of the oRNA or the payload or cargo. Non-limiting examples of the sequence to initiate degradation includes, but is not limited to, riboswitch aptazyme and miRNA binding sites.

In some embodiments, a regulatory element can modulate translation of a coding region of interest encoded on the oRNA. The modulation can create an increase (enhancer) or decrease (suppressor) in the expression of the coding region of interest. The regulatory element may be located adjacent to the CROI (e.g., on one side or both sides of the CROI).

### Translation Initiation Sequence

In some embodiments, a translation initiation sequence functions as a regulatory element. In some embodiments, the translation initiation sequence comprises an AUG/ATG codon. In some embodiments, a translation initiation sequence comprises any eukaryotic start codon such as, but not limited to, AUG/ATG, CUG/CTG, GUG/GTG, UUG/TTG, ACG, AUC/ATC, AUU, AAG, AUA/ATA, or AGG. In some embodiments, a translation initiation sequence comprises a Kozak sequence. In some embodiments, translation begins at an alternative translation initiation sequence, e.g., translation initiation sequence other than AUG/ATG codon, under selective conditions, e.g., stress induced conditions. As a non-limiting example, the translation of the circular polyribonucleotide may begin at alternative translation initiation sequence, such as ACG. As another non-limiting example, the circular polyribonucleotide translation may begin at alternative translationinitiation sequence, CUG/CTG. As another non-limiting example, the translation may begin at alternative translation initiation sequence, GUG/GTG. As yet another non-limiting example, the translation may begin at a repeat-associated non-AUG (RAN) sequence,such as an alternative translation initiation sequence that includes short stretches of repetitive RNA e.g. CGG, GGGGCC, CAG, CTG.

In some embodiments, the oRNA encodes a polypeptide or peptide and may comprise a translation initiation sequence. The translation initiation sequence may comprise, but is not limited to a start codon, a non-coding start codon, a Kozak sequence or a Shine-Dalgarno sequence. The translation initiation sequence may be located adjacent to the payload or cargo (e.g., on one side or both sides of the coding region of interest).

In some embodiments, the translation initiation sequence provides conformational flexibility to the oRNA. In some embodiments, the translation initiation sequence is within a substantially single stranded region of the ORNA.

The oRNA may include more than 1 start codon such as, but not limited to, at least 2, at least 3, at least 4, at least 5, at least 6, at least 7, at least 8, at least 9, at least 10, at least 11, at least 12, at least 13, at least 14, at least 15 or more than 15 start codons. Translation may initiate on the first start codon or may initiate downstream of the first start codon.

In some embodiments, the oRNA may initiate at a codon which is not the first start codon, e.g., AUG. Translation of the circular polyribonucleotide may initiate at an alternative translation initiation sequence, such as, but not limited to, ACG, AGG, AAG, CUG/CTG, GUG/GTG, AUA/ATA, AUU/ATT, UUG/TTG. In some embodiments, translation begins at an alternative translation initiation sequence under selective conditions, e.g., stress induced conditions. As a non-limiting example, the translation of the oRNA may begin at alternative translation initiation sequence, such as ACG. As another non-limiting example, the oRNA translation may begin at alternative translation initiation sequence, CUG/CTG. As yet another non-limiting example, the oRNA translation may begin at alternative translation initiation sequence, GTG/GUG. As yet another non-limiting example, the oRNA may begin translation at a repeat-associated non-AUG (RAN) sequence, such as an alternative translation initiation sequence that includes short stretches of repetitive RNA e.g. CGG, GGGGCC, CAG, CTG.

### IRES Sequences

In some embodiments, the oRNA described herein comprises an internal ribosome entry site (IRES) element capable of engaging an eukaryotic ribosome. In some embodiments, the IRES element is at least about 5 nucleotides, at least about 8 nucleotides, at least about 9 nucleotides, at least about 10 nucleotides, at least about 15 nucleotides, at least about 20 nucleotides, at least about 25 nucleotides, at least about 30 nucleotides, at least about 40 nucleotides, at least about 50 nucleotides, at least about 100 nucleotides, at least about 200 nucleotides, at least about 250 nucleotides, at least about 350 nucleotides, or at least about 500 nucleotides. In one embodiment, the IRES element is derived from the DNA of an organism including, but not limited to, a virus, a mammal, and a Drosophila. Such viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, the IRES element is at least partially derived from a virus, for instance, it can be derived from a viral IRES element, such as ABPV_IGRpred, AEV, ALPV_IGRpred, BQCV_IGRpred, BVDV1_1-385, BVDV1_29-391, CrPV_5NCR, CrPV_IGR, crTMV_IREScp, crTMV_IRESmp75, crTMV_IRESmp228, crTMV_IREScp, crTMV_IREScp, CSFV, CVB3, DCV_IGR, EMCV-R, EoPV_5NTR, ERAV 245-961, ERBV 162-920, EV71_1-748, FeLV-Notch2, FMDV_type_C, GBV-A, GBV-B, GBV-C, gypsy_env, gypsyD5, gypsyD2, HAV_HM175, HCV_type_la, HiPV_IGRpred, HIV-1, HoCV1_IGRpred, HRV-2, IAPV_IGRpred, idefix, KBV_IGRpred, LINE-1_ORF1-101_to_-1, LINE-1_ORF1-302_to_-202, LINE-1_ORF2-138_to_-86, LINE-1_ORF1_-44to_-1, PSIV_IGR, PV_type1_Mahoney,PV_type3_Leon, REV-A, RhPV_5NCR, RhPV_IGR, SINV1_IGRpred, SV40_661-830, TMEV, TMV_UI_IRESmp228, TRV_5NTR, TrV_IGR, or TSV_IGR. In some embodiments, the IRES element is at least partially derived from a cellular IRES, such as AML1/RUNX1, Antp-D, Antp-DE, Antp-CDE, Apaf-1, Apaf-1, AQP4, ATIR_var1, ATIR_var2, ATIR_var3, ATIR_var4, BAG1_p36delta236 nt, BAG1_p36, BCL2, BiP_-222_-3, c-IAP1_285-1399, c-IAP1_1313-1462, c-jun, c-myc, Cat-1224, CCND1, DAPS, elF4G, eIF4GI-ext, elF4GII, elF4GII-long, ELG1, ELH, FGF1A,FMR1, Gtx-133-141, Gtx-1-166, Gtx-1-120, Gtx-1-196, hairless, HAP4, HIF1a, hSNM1, Hsp101, hsp70, hsp70, Hsp90, IGF2_leader2, Kv1.4_1.2, L-myc, LamB1_-335_-1, LEF1, MNT_75-267, MNT_36-160, MTG8a, MYB, MYT2_997-1152, n-MYC, NDST1, NDST2, NDST3, NDST4L, NDST4S, NRF_-653_-17, NtHSF1, ODC1, p27kip1, 03_128-269, PDGF2/c-sis, Pim-1, PITSLRE_p58, Rbm3, reaper, Scamper, TFIID, TIF4631, Ubx_1-966, Ubx_373-961, UNR, Ure2, UtrA, VEGF-A-133-1, XIAP_5-464, XIAP_305-466, or YAP1.

In another embodiment, the IRES is an IRES sequence from Coxsackievirus B3 (CVB3), the protein coding region encodes Guassia luciferase (Gluc) and the spacer sequences are polyA-C.

In some embodiments, the IRES, if present, is at least about 50 nucleotides in length. In one embodiment, the vector comprises an IRES that comprises a natural sequence. In one embodiment, the vector comprises an IRES that comprises a synthetic sequence.

An IRES may act as the sole ribosome binding site, or may serve as one of multiple ribosome binding sites of an mRNA. A polynucleotide containing more than one functional ribosome binding site may encode several peptides or polypeptides that are translated independently by the ribosomes (e.g., multicistronic mRNA). When polynucleotides are provided with an IRES, further optionally provided is a second translatable region. Examples of IRES sequences that can be used according to the present disclosure include without limitation, those from picornaviruses (e.g., FMDV), pest viruses (CFFV), polio viruses (PV), encephalomyocarditis viruses (ECMV), foot- and mouth disease viruses (FMDV), hepatitis C viruses (HCV), classical Swine fever viruses (CSFV), murine leukemia virus (MLV), simian immune deficiency viruses (SIV) or cricket paralysis viruses (CrPV).

### Termination Element

In some embodiments, the oRNA includes one or more coding regions of interest (i.e., also referred to as product expression sequences) which encode polypeptides of interest, including but not limited to nucleobase editing system and therapeutic proteins. In various embodiments, the product expression sequences may or may not have a termination element.

In some embodiments, the oRNA includes one or more product expression sequences that lack a termination element, such that the oRNA is continuously translated.

Exclusion of a termination element may result in rolling circle translation or continuous expression of the encoded peptides or polypeptides as the ribosome will not stall or fall-off. In such an embodiment, rolling circle translation expresses continuously through the product expression sequence.

In some embodiments, one or more product expression sequences in the ORNA comprise a termination element.

In some embodiments, not all of the product expression sequences in the ORNA comprise a termination element. In such instances, the product expression sequence may fall off the ribosome when the ribosome encounters the termination element and terminates translation.

### Rolling Circle Translation

In some embodiments, once translation of the oRNA is initiated, the ribosome bound to the oRNA does not disengage from the oRNA before finishing at least one round of translation of the oRNA. In some embodiments, the oRNA as described herein is competent for rolling circle translation. In some embodiments, during rolling circle translation, once translation of the oRNA is initiated, the ribosome bound to the oRNA does not disengage from the oRNA before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 10.sup.5 rounds, or at least 10.sup.6 rounds of translation of the ORNA.

In some embodiments, the rolling circle translation of the oRNA leads to generation of polypeptide that is translated from more than one round of translation of the ORNA. In some embodiments, the oRNA comprises a stagger element, and rolling circle translation of the oRNA leads to generation of polypeptide product that is generated from a single round of translation or less than a single round of translation of the oRNA.

### Circularization

In one embodiment, a linear RNA may be cyclized, or concatemerized. In some embodiments, the linear RNA may be cyclized in vitro prior to formulation and/or delivery. In some embodiments, the linear RNA may be cyclized within a cell.

In some embodiments, the mechanism of cyclization or concatemerization may occur through at least 3 different routes: 1) chemical, 2) enzymatic, and 3) ribozyme catalyzed. The newly formed 5′-/3′-linkage may be intramolecular or intermolecular.

In the first route, 5′-end and the 3 ′-end of the nucleic acid contain chemically reactive groups that, when close together, form a new covalent linkage between the 5′-end and the 3 ′-end of the molecule. The 5′-end may contain an NHS-ester reactive group and the 3 ′-end may contain a 3′-amino-terminated nucleotide such that in an organic solvent 3′-amino-terminated nucleotide on the 3 ′-end of a synthetic mRNA molecule will undergo a nucleophilic attack on the 5′-NHS-ester moiety forming a new 5 ′-/3′-amide bond.

In the second route, T4 RNA ligase may be used to enzymatically link a 5′-phosphorylated nucleic acid molecule to the 3′-hydroxyl group of a nucleic acid forming a new phosphorodiester linkage. In an example reaction, \hat{ }g of a nucleic acid molecule is incubated at 37° C. for 1 hour with 1-10 units of T4 RNA ligase (New England Biolabs, Ipswich, MA) according to the manufacturer's protocol. The ligation reaction may occur in the presence of a split oligonucleotide capable of base-pairing with both 5′- and 3′-region in juxtaposition to assist the enzymatic ligation reaction.

In the third route, either the 5 ′- or 3′-end of the cDNA template encodes a ligase ribozyme sequence such that during in vitro transcription, the resultant nucleic acid molecule can contain an active ribozyme sequence capable of ligating the 5 ′-end of a nucleic acid molecule to the 3′-end of a nucleic acid molecule. The ligase ribozyme may be derived from the Group I Intron, Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, the oRNA is made via circularization of a linear RNA.

In some embodiments, the following elements are operably connected to each other and, in some embodiments, arranged in the following sequence: a.) a 5′ homology arm, b.) a 3′ group I intron fragment containing a 3′ splice site dinucleotide, c.) a protein coding or noncoding region, d.) a 5′ group I intron fragment containing a 5′ splice site dinucleotide, and e.) a 3′ homology arm. In certain embodiments said vector allows production of a circular RNA that is translatable and/or biologically active inside eukaryotic cells. In some embodiments, the biologically active RNA is, for example, an miRNA sponge, or long noncoding RNA.

In some embodiments, the following elements are operably connected to each other and arranged in the following sequence: a.) a 5′ homology arm, b.) a 3′ group I intron fragment containing a 3′ splice site dinucleotide, c.) optionally, a 5′ spacer sequence, d.) optionally, an internal ribosome entry site (IRES), e.) a protein coding or noncoding region, f.) optionally, a 3′ spacer sequence, g.) a 5′ group I intron fragment containing a 5′ splice site dinucleotide, and h.) a 3′ homology arm. In certain embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) an internal ribosome entry site (IRES), e.) a protein coding or noncoding region, f.) a 5′ group I intron fragment containing a 5′ splice site dinucleotide, and g.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) a protein coding or noncoding region, e.) a 3′ spacer sequence, f.) a 5′ group I intron fragment containing a 5′ splice site dinucleotide, and g.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) an internal ribosome entry site (IRES), d.) a protein coding or noncoding region, e.) a 3′ spacer sequence, f) a 5′ group I intron fragment containing a 5′ splice site dinucleotide, and g.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a protein coding or noncoding region, d.) a 3′ spacer sequence, e.) a 5′ group I intron fragment containing a 5′ splice site dinucleotide, and f.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) a protein coding or noncoding region, e.) a 5′ group I intron fragment containing a 5′ splice site dinucleotide, and f.) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) an internal ribosome entry site (IRES), d.) a protein coding or noncoding region, e.) a 5′ group I intron fragment containing a 5′ splice site dinucleotide, and f) a 3′ homology arm. In some embodiments, said vector allows production of a circular RNA that is translatable and/or biologically active inside eukaryotic cells.

In some embodiments, the following elements are operably connected to each other and arranged in the following sequence: a.) a 5′ homology arm, b.) a 3′ group I intron fragment containing a 3′ splice site dinucleotide, c.) a 5′ spacer sequence, d.) an internal ribosome entry site (IRES), e.) a protein coding or noncoding region, f) a 3′ spacer sequence, g.) a 5′ group I intron fragment containing a 5′ splice site dinucleotide, and h.) a 3′ homology arm. In some embodiments, said vector allowing production of a circular RNA that is translatable and/or biologically active inside eukaryotic cells.

In one embodiment, 3′ group I intron fragment and/or 5′ group I intron fragment is from a Cyanobacterium Anabaena sp. pre-tRNA-Leu gene or T4 phage Td gene.

In one embodiment, 3′ group I intron fragment and/or 5′ group I intron fragment is from a Cyanobacterium Anabaena sp. pre-tRNA-Leu gene.

In one embodiment, the protein coding region encodes a protein of eukaryotic or prokaryotic origin. In another embodiment, the protein coding region encodes human protein or non-human protein. In some embodiments, the protein coding region encodes one or more antibodies. For example, in some embodiments, the protein coding region encodes human antibodies. In one embodiment, the protein coding region encodes a protein selected from hFIX, SP-B, VEGF-A, human methylmalonyl-CoA mutase (hMUT), CFTR, cancer self-antigens, and additional gene editing enzymes like Cpf1, zinc finger nucleases (ZFNs) and transcription activator-like effector nucleases (TALENs). In another embodiment, the protein coding region encodes a protein for therapeutic use. In one embodiment, the human antibody encoded by the protein coding region is an anti-HIV antibody. In one embodiment, the antibody encoded by the protein coding region is a bispecific antibody. In one embodiment, the bispecific antibody is specific for CD19 and CD22. In another embodiment, the bispecific antibody is specific for CD3 and CLDN6. In one embodiment, the protein coding region encodes a protein for diagnostic use. In one embodiment, the protein coding region encodes Gaussia luciferase (Gluc), Firefly luciferase (Fluc), enhanced green fluorescent protein (eGFP), human erythropoietin (hEPO), or Cas9 endonuclease.

In one embodiment, 5′ homology arm is about 5-50 nucleotides in length. In another embodiment, 5′ homology arm is about 9-19 nucleotides in length. In some embodiments, 5′ homology arm is at least 5, 6, 7, 8, 9, 10, 11, 12, 13, 14, 15, 16, 17, 18 or 19 nucleotides in length. In some embodiments, 5′ homology arm is no more than 50, 45, 40, 35, 30, 25 or 20 nucleotides in length. In some embodiments, 5′ homology arm is 5, 6, 7, 8, 9, 10, 11, 12, 13, 14, 15, 16, 17, 18 or 19 nucleotides in length.

In one embodiment, 3′ homology arm is about 5-50 nucleotides in length. In another embodiment, 3′ homology arm is about 9-19 nucleotides in length. In some embodiments, 3′ homology arm is at least 5, 6, 7, 8, 9, 10, 11, 12, 13, 14, 15, 16, 17, 18 or 19 nucleotides in length. In some embodiments, 3′ homology arm is no more than 50, 45, 40, 35, 30, 25 or 20 nucleotides in length. In some embodiments, 3′ homology arm is 5, 6, 7, 8, 9, 10, 11, 12, 13, 14, 15, 16, 17, 18 or 19 nucleotides in length.

In one embodiment, 5′ spacer sequence is at least 10 nucleotides in length. In another embodiment, 5′ spacer sequence is at least 15 nucleotides in length. In a further embodiment, 5′ spacer sequence is at least 30 nucleotides in length. In some embodiments, 5′ spacer sequence is at least 7, 8, 9, 10, 11, 12, 13, 14, 15, 16, 17, 18, 19, 20, 25 or 30 nucleotides in length. In some embodiments, 5′ spacer sequence is no more than 100, 90, 80, 70, 60, 50, 45, 40, 35 or 30 nucleotides in length. In some embodiments the 5′ spacer sequence is between 20 and 50 nucleotides in length. In certain embodiments, 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, 5′ spacer sequence is a polyA sequence. In another embodiment, 5′ spacer sequence is a polyA-C sequence.

In one embodiment, 3′ spacer sequence is at least 10 nucleotides in length. In another embodiment, 3′ spacer sequence is at least 15 nucleotides in length. In a further embodiment, 3′ spacer sequence is at least 30 nucleotides in length. In some embodiments, 3′ spacer sequence is at least 7, 8, 9, 10, 11, 12, 13, 14, 15, 16, 17, 18, 19, 20, 25 or 30 nucleotides in length. In some embodiments, 3′ spacer sequence is no more than 100, 90, 80, 70, 60, 50, 45, 40, 35 or 30 nucleotides in length. In some embodiments the 3′ spacer sequence is between 20 and 50 nucleotides in length. In certain embodiments, 3′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, 3′ spacer sequence is a polyA sequence. In another embodiment, 5′ spacer sequence is a polyA-C sequence.

### Extracellular Circularization

In some embodiments, the linear RNA is cyclized, or concatemerized using a chemical method to form an oRNA. In some chemical methods, 5′-end and 3′-end of the nucleic acid (e.g., a linear RNA) includes chemically reactive groups that, when close together, may form a new covalent linkage between 5′-end and 3′-end of the molecule. The 5′-end may contain an NHS-ester reactive group and 3′-end may contain a 3′-amino-terminated nucleotide such that in an organic solvent 3′-amino-terminated nucleotide on the 3′-end of a linear RNA will undergo a nucleophilic attack on the 5′-NHS-ester moiety forming a new 5′-/3′-amide bond.

In one embodiment, a DNA or RNA ligase may be used to enzymatically link a 5′-phosphorylated nucleic acid molecule (e.g., a linear RNA) to the 3′-hydroxyl group of a nucleic acid (e.g., a linear nucleic acid) forming a new phosphorodiester linkage. In an example reaction, a linear RNA is incubated at 37 C for 1 hour with 1-10 units of T4 RNA ligase according to the manufacturer's protocol. The ligation reaction may occur in the presence of a linear nucleic acid capable of base-pairing with both 5′- and 3′-region in juxtaposition to assist the enzymatic ligation reaction. In one embodiment, the ligation is splint ligation where a single stranded polynucleotide (splint), like a single stranded RNA, can be designed to hybridize with both termini of a linear RNA, so that the two termini can be juxtaposed upon hybridization with the single-stranded splint. Splint ligase can thus catalyze the ligation of the juxtaposed two termini of the linear RNA, generating an oRNA.

In one embodiment, a DNA or RNA ligase may be used in the synthesis of the ORNA. As a non-limiting example, the ligase may be a circ ligase or circular ligase.

In one embodiment, either 5′- or 3′-end of the linear RNA can encode a ligase ribozyme sequence such that during in vitro transcription, the resultant linear RNA includes an active ribozyme sequence capable of ligating 5′-end of the linear RNA to the 3′-end of the linear RNA. The ligase ribozyme may be derived from the Group I Intron, Hepatitis Delta Virus, Hairpin ribozyme or may be selected by SELEX (systematic evolution of ligands by exponential enrichment).

In one embodiment, a linear RNA may be cyclized or concatemerized by using at least one non-nucleic acid moiety. In one aspect, the at least one non-nucleic acid moiety may react with regions or features near the 5′ terminus and/or near the 3′ terminus of the linear RNA in order to cyclize or concatermerize the linear RNA. In another aspect, the at least one non-nucleic acid moiety may be located in or linked to or near the 5′ terminus and/or the 3′ terminus of the linear RNA. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In one embodiment, a linear RNA may be cyclized or concatemerized due to a non-nucleic acid moiety that causes an attraction between atoms, molecular surfaces at, near or linked to the 5′ and 3′ ends of the linear RNA. As a non-limiting example, one or more linear RNA may be cyclized or concatemerized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In one embodiment, the linear RNA may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′ terminus may associate with each other causing a linear RNA to cyclize or concatemerize. In another aspect, the peptides covalently linked to the ribozyme RNA near the 5′ terminus and the 3′ terminus may cause the linear RNA to cyclize or concatemerize after being subjected to ligated using various methods known in the art such as, but not limited to, protein ligation.

In some embodiments, the linear RNA may include a 5′ triphosphate of the nucleic acid converted into a 5′ monophosphate, e.g., by contacting 5′ triphosphate with RNA 5′ pyrophosphohydrolase (RppH) or an ATP diphosphohydrolase (apyrase). Alternately, converting 5′ triphosphate of the linear RNA into a 5′ monophosphate may occur by a two-step reaction comprising: (a) contacting 5′ nucleotide of the linear RNA with a phosphatase (e.g., Antarctic Phosphatase, Shrimp Alkaline Phosphatase, or Calf Intestinal Phosphatase) to remove all three phosphates; and (b) contacting 5′ nucleotide after step (a) witha kinase (e.g., Polynucleotide Kinase) that adds a single phosphate.

In some embodiments, RNA may be circularized using the methods described in WO2017222911 and WO2016197121, the contents of each of which are herein incorporated by reference in their entirety.

In some embodiments, RNA may be circularized, for example, by back splicing of a non-mammalian exogenous intron or splint ligation of 5′ and 3 ′ends of a linear RNA. In one embodiment, the circular RNA is produced from a recombinant nucleic acid encoding the target RNA to be made circular. As a non-limiting example, the method comprises: a) producing a recombinant nucleic acid encoding the target RNA to be made circular, wherein the recombinant nucleic acid comprises in 5′ to 3 ′order: i) a 3′ portion of an exogenous intron comprising a 3′ splice site, ii) a nucleic acid sequence encoding the target RNA, and iii) a 5 ′portion of an exogenous intron comprising a 5′ splice site; b) performing transcription, whereby RNA is produced from the recombinant nucleic acid; and c) performing splicing of the RNA, whereby the RNA circularizes to produce a oRNA.

While not wishing to be bound by theory, circular RNAs generated with exogenous introns are recognized by the immune system as “non-self” and trigger an innate immune response. On the other hand, circular RNAs generated with endogenous introns are recognized by the immune system as “self” and generally do not provoke an innate immune response, even if carrying an exon comprising foreign RNA.

Accordingly, circular RNAs can be generated with either an endogenous or exogenous intron to control immunological self/non-self discrimination as desired. Numerous intron sequences from a wide variety of organisms and viruses are known and include sequences derived from genes encoding proteins, ribosomal RNA (rRNA), or transfer RNA (tRNA).

Circular RNAs can be produced from linear RNAs in a number of ways. In some embodiments, circular RNAs are produced from a linear RNA by backsplicing of a downstream 5′ splice site (splice donor) to an upstream 3′ splice site (splice acceptor). Circular RNAs can be generated in this manner by any nonmammalian splicing method. For example, linear RNAs containing various types of introns, including self-splicing group I introns, self-splicing group II introns, spliceosomal introns, and tRNA introns can be circularized. In particular, group I and group II introns have the advantage that they can be readily used for production of circular RNAs in vitro as well as in vivo because of their ability to undergo self-splicing due to their autocatalytic ribozyme activity.

In some embodiments, circular RNAs can be produced in vitro from a linear RNA by chemical or enzymatic ligation of 5′ and 3′ ends of the RNA. Chemical ligation can be performed, for example, using cyanogen bromide (BrCN) or ethyl-3-(3′-dimethylaminopropyl) carbodiimide (EDC) for activation of a nucleotide phosphomonoester group to allow phosphodiester bond formation. See e.g., Sokolova (1988) FEBS Lett 232:153-155; Dolinnaya et al. (1991)Nucleic Acids Res., 19:3067-3072; Fedorova (1996) Nucleosides Nucleotides Nucleic Acids 15:1 137-1 147; herein incorporated by reference. Alternatively, enzymatic ligation can be used to circularize RNA. Exemplary ligases that can be used include T4 DNA ligase (T4 Dnl), T4 RNA ligase 1 (T4 Rnl 1), and T4 RNA ligase 2 (T4 Rnl 2).

In some embodiments, splint ligation using an oligonucleotide splint that hybridizes with the two ends of a linear RNA can be used to bring the ends of the linear RNA together for ligation. Hybridization of the splint, which can be either a DNA or a RNA, orientates the 5 ′-phosphate and 3′—OH of the RNA ends for ligation. Subsequent ligation can be performed using either chemical or enzymatic techniques, as described above. Enzymatic ligation can be performed, for example, with T4 DNA ligase (DNA splint required), T4 RNA ligase 1 (RNA splint required) or T4 RNA ligase 2 (DNA or RNA splint). Chemical ligation, such as with BrCN or EDC, in some cases is more efficient than enzymatic ligation if the structure of the hybridized splint-RNA complex interferes with enzymatic activity.

In some embodiments, the oRNA may further comprise an internal ribosome entry site (IRES) operably linked to an RNA sequence encoding a polypeptide. Inclusion of an IRES permits the translation of one or more open reading frames from a circular RNA. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996)229:295-298; Rees et al., BioTechniques (1996)20:102-110; Kobayashi et al., BioTechniques (1996)21:399-402; and Mosser et al., BioTechniques 1997 22 150-161).

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%.

### Splicing Element

In some embodiments, the oRNA includes at least one splicing element. The splicing element can be a complete splicing element that can mediate splicing of the oRNA or the spicing element can be a residual splicing element from a completed splicing event. For instance, in some cases, a splicing element of a linear RNA can mediate a splicing event that results in circularization of the linear RNA, thereby the resultant oRNA comprises a residual splicing element from such splicing-mediated circularization event. In some cases, the residual splicing element is not able to mediate any splicing. In other cases, the residual splicing element can still mediate splicing under certain circumstances. In some embodiments, the splicing element is adjacent to at least one expression sequence. In some embodiments, the oRNA includes a splicing element adjacent each expression sequence. In some embodiments, the splicing element is on one or both sides of each expression sequence, leading to separation of the expression products, e.g., peptide(s) and or polypeptide(s).

In some embodiments, the oRNA includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, Alu elements in flanking introns, and motifs found in (suptable4 enriched motifs) cis -sequence elements proximal to backsplice events such as sequences in the 200 bp preceding (upstream of) or following (downstream from) a backsplice site with flanking exons. In some embodiments, the oRNA includes at least one repetitive nucleotide sequence described elsewhere herein as an internal splicing element. In such embodiments, the repetitive nucleotide sequence may include repeated sequences from the Alu family of introns. See, e.g., U.S. Pat. No. 11,058,706.

In some embodiments, the oRNA may include canonical splice sites that flank head-to-tail junctions of the ORNA.

In some embodiments, the oRNA may include a bulge-helix-bulge motif, comprising a 4-base pair stem flanked by two 3-nucleotide bulges. Cleavage occurs at a site in the bulge region, generating characteristic fragments with terminal 5′-hydroxyl group and 2′, 3′-cyclic phosphate. Circularization proceeds by nucleophilic attack of 5′-OH group onto the 2′, 3′-cyclic phosphate of the same molecule forming a 3′, 5′-phosphodiester bridge.

In some embodiments, the oRNA may include a sequence that mediates self-ligation. Non-limiting examples of sequences that can mediate self-ligation include a self-circularizing intron, e.g., a 5′ and 3′ slice junction, or a self-circularizing catalytic intron such as a Group I, Group II or Group III Introns. Non-limiting examples of group I intron self-splicing sequences may includeself-splicing permuted intron-exon sequences derived from T4 bacteriophage gene td, and the intervening sequence (IVS) rRNA of Tetrahymena.

### Other Circularization Methods

In some embodiments, linear RNA may include complementary sequences, including either repetitive or nonrepetitive nucleic acid sequences within individual introns or across flanking introns. In some embodiments, the oRNA includes a repetitive nucleic acid sequence. In some embodiments, the repetitive nucleotide sequence includes poly CA or poly UG sequences. In some embodiments, the oRNA includes at least one repetitive nucleic acid sequence that hybridizes to a complementary repetitive nucleic acid sequence in another segment of the oRNA, with the hybridized segment forming an internal double strand. In some embodiments, repetitive nucleic acid sequences and complementary repetitive nucleic acid sequences from two separate oRNA that hybridize to generate a single oRNA, with the hybridized segments forming internal double strands. In some embodiments, the complementary sequences are found at 5′ and 3′ ends of the linear RNA. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the oRNA. Such methods may include, but are not limited to click chemistry (e.g., alkyne- and azide-based methods, or clickable bases), olefin metathesis, phosphoramidate ligation, hemiaminal-imine crosslinking, base modification, and any combination thereof. In some embodiments, enzymatic methods of circularization may be used to generate the oRNA. In some embodiments, a ligation enzyme, e.g., DNA or RNA ligase, may be used to generate a template of the oRNA or complement, a complementary strand of the ORNA, or the oRNA. Any of the circular polynucleotides as taught in for example U.S. Pat. No. 10,709,779, which is incorporated by reference herein in its entirety, may be used herein. In addition, any of the circular RNAs, methods for making circular RNAs, circular RNA compositions that are described in the following publications are contemplated herein and are incorporated by reference in their entireties are part of the instant specification: US-Patents U.S. Pat. Nos. 11,352,640, 11,352,641, 11,203,767, 10,683,498, 5,773,244, and 5,766,903; US Application Publications US 2022/0177540, US 2021/0371494, US 2022/0090137, US 2019/0345503, and US 2015/0299702; and PCT Application Publications WO 2021/226597, WO 2019/236673, WO 2017/222911, WO2016/187583, WO2014/082644 and WO 1997/007825.

### D. Methods of Use

The LNP-based compositions described herein may be used to deliver a TnpB nucleobase editing system to a cell or tissue of interest. In certain embodiments, the LNP-based compositions described herein are useful for executing one or more edits, modifications or alterations to one or more targeted genes of interest. In certain embodiments, the one or more edits, modifications or alterations to the one or more targeted genes of interest are capable of treating a disease or disorder in a patient in need thereof. The following is a description of various non-limiting methods of use.

### 1. Gene Editing

In some embodiments, the TnpB gene editing systems described herein (including any described or contemplated format, such as a TnpB base editor, TnpB prime editor, or TnpB retron editor) are used for genome editing at a desired site. In some embodiments, the TnpB systems include a DNA donor template comprising an edited sequence.

In some embodiments, the DNA donor template has 10-100 or more bp of homologous nucleic acid sequence to the genome on both sides of the desired edit. The desired edit (insertion, deletion, or mutation) is in between the homologous sequence.

In some embodiments, DNA donor template comprise a sequence comprising an intended genome edit flanked by a pair of homology arms responsible for targeting the donor polynucleotide to the target locus to be edited in a cell. The donor polynucleotide typically comprises a 5′ homology arm that hybridizes to a 5′ genomic target sequence and a 3′ homology arm that hybridizes to a 3′ genomic target sequence. The homology arms are referred to herein as 5′ and 3′ (i.e., upstream and downstream) homology arms, which relate to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively.

The homology arm must be sufficiently complementary for hybridization to the target sequence to mediate homologous recombination between the donor polynucleotide and genomic DNA at the target locus. For example, a homology arm may comprise a nucleotide sequence having at least about 80-100% sequence identity to the corresponding genomic target sequence, including any percent identity within this range, such as at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity thereto, wherein the nucleotide sequence comprising the intended edit can be integrated into the genomic DNA by HDR at the genomic target locus recognized (i.e., having sufficient complementary for hybridization) by 5′ and 3′ homology arms.

In some embodiments, the corresponding homologous nucleotide sequences in the genomic target sequence (i.e., the “5′ target sequence” and “3′ target sequence”) flank a specific site for cleavage and/or a specific site for introducing the intended edit. The distance between the specific cleavage site and the homologous nucleotide sequences (e.g., each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate. In some embodiments, the donor polynucleotide is substantially identical to the target genomic sequence, across its entire length except for the sequence changes to be introduced to a portion of the genome that encompasses both the specific cleavage site and the portions of the genomic target sequence to be altered.

A homology arm can be of any length, e.g. 10 nucleotides or more, 15 nucleotides or more, 20 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000 nucleotides (1 kb) or more, 5000 nucleotides (5 kb) or more, 10000 nucleotides (10 kb) or more, etc. In some instances, 5′ and 3′ homology arms are substantially equal in length to one another. However, in some instances 5′ and 3′ homology arms are not necessarily equal in length to one another. For example, one homology arm may be 30% shorter or less than the other homology arm, 20% shorter or less than the other homology arm, 10% shorter or less than the other homology arm, 5% shorter or less than the other homology arm, 2% shorter or less than the other homology arm, or only a few nucleotides less than the other homology arm. In other instances, 5′ and 3′ homology arms are substantially different in length from one another, e.g. one may be 40% shorter or more, 50% shorter or more, sometimes 60% shorter or more, 70% shorter or more, 80% shorter or more, 90% shorter or more, or 95% shorter or more than the other homology arm.

The DNA donor template may be used in combination with an RNA-guided nuclease, which is targeted to a particular genomic sequence (i.e., genomic target sequence to be modified) by a guide RNA. A target-specific guide RNA comprises a nucleotide sequence that is complementary to a genomic target sequence, and thereby mediates binding of the nuclease-gRNA complex by hybridization at the target site. For example, the gRNA can be designed with a sequence complementary to the sequence of a minor allele to target the nuclease-gRNA complex to the site of a mutation. The mutation may comprise an insertion, a deletion, or a substitution. For example, the mutation may include a single nucleotide variation, gene fusion, translocation, inversion, duplication, frameshift, missense, nonsense, or other mutation associated with a phenotype or disease of interest. The targeted minor allele may be a common genetic variant or a rare genetic variant. In some embodiments, the gRNA is designed to selectively bind to a minor allele with single base-pair discrimination, for example, to allow binding of the nuclease-gRNA complex to a single nucleotide polymorphism (SNP). In particular, the gRNA may be designed to target disease-relevant mutations of interest for the purpose of genome editing to remove the mutation from a gene. Alternatively, the gRNA can be designed with a sequence complementary to the sequence of a major or wild-type allele to target the nuclease-gRNA complex to the allele for the purpose of genome editing to introduces a mutation into a gene in the genomic DNA of the cell, such as an insertion, deletion, or substitution. Such genetically modified cells can be used, for example, to alter phenotype, confer new properties, or produce disease models for drug screening.

In some embodiments, the TnpB editing systems can comprise one or more additional RNA-guided nuclease used for genome modification is a clustered regularly interspersed short palindromic repeats (CRISPR) system Cas nuclease. Any RNA-guided Cas nuclease capable of catalyzing site-directed cleavage of DNA to allow integration of donor polynucleotides by the HDR mechanism can be used in genome editing, including CRISPR system Class 1, Type I, II, or III Cas nucleases; Class 2, Type II nuclease (such as Cas9); a Class 2, Type V nuclease (such as Cpf1), or a Class 2, Type VI nuclease (such as C2c2). Examples of Cas proteins include Cas1, CaslB, Cas2, Cas3, Cas4, Cas5, Cas5e (CasD), Cas6, Cas6e, Casof, Cas7, Cas8al, Cas8a2, Cas8b, Cas8c, Cas9 (Csn1 or Csx12), CasIO, CaslOd, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and Cul966, and homologs or modified versions thereof.

In some embodiments, a Class 1, type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP 002989955, WP_038434062, WP_011528583); Campylobacter jejuni (WP_022552435, YP 002344900), Campylobacter coli (WP 060786116); Campylobacter fetus (WP 059434633); Corynebacterium ulcerans (NC_015683, NC_017317); Corynebacterium diphtheria (NC_016782, NC_016786); Enterococcus faecalis (WP 033919308); Spiroplasma syrphidicola (NC 021284); Prevotella intermedia (NC 017861); Spiroplasma taiwanense (NC 021846); Streptococcus iniae (NC 021314); Belliella baltica (NC 018010); Psychroflexus torquisl (NC O 18721); Streptococcus thermophilus (YP 820832), Streptococcus mutans (WP 061046374, WP 024786433); Listeria innocua (NP 472073); Listeria monocytogenes (WP 061665472); Legionella pneumophila (WP 062726656); Staphylococcus aureus (WP_001573634); Francisella tularensis (WP_032729892, WP_014548420), Enterococcus faecalis (WP 033919308); Lactobacillus rhamnosus (WP 048482595, WP_032965177); and Neisseria meningitidis (WP_061704949, YP_002342100); all of which sequences (as entered by the date of filing of this application) are herein incorporated by reference in their entireties. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014)Nucleic Acids Res. 42 (4): 2577-90; Kapitonov et al. (2015) J. Bacterid. 198 (5): 797-807, Shmakov et al. (2015) Mol. Cell. 60 (3): 385-397, and Chylinski et al. (2014)Nucleic Acids Res. 42 (10): 6091-6105); for sequence comparisons and a discussion of genetic diversity and phylogenetic analysis of Cas9.

The genomic target site will typically comprise a nucleotide sequence that is complementary to the gRNA and may further comprise a protospacer adjacent motif (PAM). In some embodiments, the target site comprises 20-30 base pairs in addition to a 3 or more base pair PAM. Typically, the first nucleotide of a PAM can be any nucleotide, while the two or more other nucleotides will depend on the specific Cas9 protein that is chosen. Exemplary PAM sequences are known to those of skill in the art and include, without limitation, NNG, NGN, NAG, and NGG, wherein N represents any nucleotide. In some embodiments, the allele targeted by a gRNA comprises a mutation that creates a PAM within the allele, wherein the PAM promotes binding of the Cas9-gRNA complex to the allele.

In some embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In another embodiment, the CRISPR nuclease from Prevotella and Francisella 1 (Cpf1, or Cas12a) is used. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015)Nature. 526 (7571): 17-17, Zetsche et al. (2015)Cell. 163 (3): 759-771, Murovec et al. (2017) Plant Biotechnol. J. 15 (8): 917-926, Zhang et al. (2017) Front. Plant Sci. 8:177, Fernandes et al. (2016) Postepy Biochem. 62 (3): 315-326; herein incorporated by reference.

C2cl (Cas12b) is another class II CRISPR/Cas system RNA-guided nuclease that may be used. C2cl, similarly to Cas9, depends on both a crRNA and tracrRNA for guidance to target sites. See, e.g., Shmakov et al. (2015) Mol Cell. 60 (3): 385-397, Zhang et al. (2017) Front Plant Sci. 8:177; herein incorporated by reference.

In yet another embodiment, an engineered RNA-guided Fokl nuclease may be used. RNA-guided Fokl nucleases comprise fusions of inactive Cas9 (dCas9) and the Fokl endonuclease (FokI-dCas9), wherein the dCas9 portion confers guide RNA-dependent targeting on Fokl. For a description of engineered RNA-guided Fold nucleases, see, e.g., Havlicek et al. (2017) Mol. Ther. 25 (2): 342-355, Pan et al. (2016) Sci Rep. 6:35794, Tsai et al. (2014)Nat Biotechnol. 32 (6): 569-576; herein incorporated by reference.

In other embodiments, any other Cas enzymes and variants described in other sections of the application (all incorporated herein) can be used similarly.

In some embodiments, the RNA-guided nuclease is provided in the form of a protein, optionally where the nuclease is complexed with a gRNA to form a ribonucleoprotein (RNP) complex. In some embodiments, the RNA-guided nuclease is provided by a nucleic acid encoding the RNA-guided nuclease, such as an RNA (e.g., messenger RNA) or DNA (expression vector). In some embodiments, the RNA-guided nuclease and the gRNA are both provided by vectors, such as the vectors and the vector system described in other parts of the application (all incorporated herein by reference). Both can be expressed by a single vector or separately on different vectors. The vectors encoding the RNA-guided nuclease and gRNA may be included in the vector system comprising the TnpB editing system msr gene, msd gene and ret gene sequences. In some embodiments, the RNA-guided nuclease is fused to the RT and/or the msDNA.

The RNP complex may be administered to a subject or delivered into a cell by methods known in the art, such as those described in U.S. Pat. No. 11,390,884, which is incorporated by reference herein in its entirety. In some embodiments, the endonuclease/gRNA ribonucleoprotein (RNP) complexes are delivered to cells by electroporation. Direct delivery of the RNP complex to a subject or cell eliminates the need for expression from nucleic acids (e.g., transfection of plasmids encoding Cas9 and gRNA). It also eliminates unwanted integration of DNA segments derived from nucleic acid delivery (e.g., transfection of plasmids encoding Cas9 and gRNA). An endonuclease/gRNA ribonucleoprotein (RNP) complex usually is formed prior to administration.

Codon usage may be optimized to further improve production of an RNA-guided nuclease and/or reverse transcriptase (RT) in a particular cell or organism. For example, a nucleic acid encoding an RNA-guided nuclease or reverse transcriptase can be modified to substitute codons having a higher frequency of usage in a yeast cell, a bacterial cell, a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease or reverse transcriptase is introduced into cells, the protein can be transiently, conditionally, or constitutively expressed in the cell.

In some embodiments, the TnpB editing system used for genome editing with nuclease genome editing systems can further include accessory or enhancer proteins for recombination. Examples of recombination enhancers can include nonhomologous end joining (NHEJ) inhibitors (e.g., inhibitor of DNA ligase IV, a KU inhibitor (e.g., KU70 or KU80), a DNA-PKc inhibitor, or an artemis inhibitor) and homologous directed repair (HDR) promoters, or both, that can enhance or improve more precise genome editing and/or the efficiency of homologous recombination. In some embodiments, the recombination accessory or enhancers can comprise C-terminal binding protein interacting protein (CtIP), cyclinB2, Rad family members (e.g. Rad50, Rad51, Rad52, etc).

CtIP is a transcription factor containing C2H2 zinc fingers that are involved in early steps of homologous recombination. Mammalian CtIP and its orthologs in other eukaryotes promote the resection of DNA double-strand breaks and are essential for meiotic recombination. HDR may be enhanced by using Cas9 nuclease associated (e.g. fused) to an N-terminal domain of CtIP, an approach that forces CtIP to the cleavage site and increases transgene integration by HDR. In some embodiments, an N-terminal fragment of CtIP, called HE for HDR enhancer, may be sufficient for HDR stimulation and requires the CtIP multimerization domain and CDK phosphorylation sites to be active. HDR stimulation by the Cas9-HE fusion depends on the guide RNA used, and therefore the guide RNA will be designed accordingly.

Using the gene editing system described herein, any target gene or sequence in a host cell can be edited or modified for a desired trait, including but not limited to: Myostatin (e.g., GDF8) to increase muscle growth; Pc POLLED to induce hairlessness; KISSIR to induce bore taint; Dead end protein (dnd) to induce sterility; Nano2 and DDX to induce sterility; CD163 to induce PRRSV resistance; RELA to induce ASFV resilience; CD18 to induce Mannheimia(Pasteurella) haemolytica resilience; NRAMPI to induce tuberculosis resilience; Negative regulators of muscle mass (e.g., Myostatin) to increase muscle mass.

### 2. Epigenetic Editing

In some embodiments, the TnpB gene editing systems described herein when including an epigenetic modifier domain can be used for genome editing at a desired site. Epigenetic modifications of DNA and histones are known for their multifaceted contributions to transcriptional regulation. As these modifications are faithfully propagated throughout DNA replication, they are considered central players in cellular memory of transcriptional states. Many efforts in the last decade have generated a vast understanding of individual epigenetic modifications and their contribution to transcriptional regulation. Epigenetic editing offers powerful tools to selectively induce epigenetic changes in a genome without altering the sequence of a nucleotide sequence as a means to regulate gene activity. The foundation of epigenetic editing is formed by the ability to generate fusion proteins of epigenetic enzymes or their catalytic domains with programmable DNA-binding platforms such as the clustered regularly interspaced short palindromic repeat (e.g., CRISPR Cas9 or TnpB) to target these to an endogenous locus of choice. The enzymatic fusion protein then dictates the initial deposited modification while subsequent cross-talk within the local chromatin environment likely influences epigenetic and transcriptional output.

Accordingly, in one aspect, the disclosure provides an epigenetic gene editing system comprising one or more epigenetic enzymes or their catalytic domains combined with a TnpB programmable nuclease, and an appropriate guide RNA for guiding the TnpB to a particular target site. In some embodiments, the TnpB may be fused to the epigenetic enzyme or a catalytic domain thereof. In other embodiments, the TnpB and the epigenetic enzyme or catalytic domain thereof are not fused but may be co-delivered. In the latter embodiment, the epigenetic enzyme or catalytic domain there may include at targeting moiety to cause it to be co-localized with the TnpB at the target site defined by the guide RNA.

Epigenetic enzymes include, but are not limited to DNA methyltransferases, histone methyltransferases, and histone deacetylases. In other embodiments, the epigenetic enzyme is histone deacetylase, histone deacetylase, histone methyl transferase, histone demethylase, DNA methyl transferase, DNA demethylase, DNA ligase, other ligases, ubiquitinase, ubiquitin ligase, phosphatase, or a phosphokinase.

In some embodiments, the DNA donor template has 10-100 or more bp of homologous nucleic acid sequence to the genome on both sides of the desired edit. The desired edit (insertion, deletion, or mutation) is in between the homologous sequence.

In still other embodiments, the LNPs may be used to deliver an epigenetic editing system. Epigenetic editors are generally composed of an epigenetic enzyme or their catalytic domain fused with a user-programmable DNA-binding protein, such as TnpB. The user-programmable DNA-binding protein (plus a guide RNA in the case of a nucleic acid programmable DNA binding protein) guides the epigenetic enzyme (e.g., a DNA methyltransferase or DNMT) to a specific site (e.g., a CpG island in a promoter region of a gene) in order to induce a change in promoter activity.

Epigenetic modifications of DNA and histones are known for their multifaceted contributions to transcriptional regulation. As these modifications are faithfully propagated throughout DNA replication, they are considered central players in cellular memory of transcriptional states. Many efforts in the last decade have generated a vast understanding of individual epigenetic modifications and their contribution to transcriptional regulation. Epigenetic editing offers powerful tools to selectively induce epigenetic changes in a genome without altering the sequence of a nucleotide sequence as a means to regulate gene activity. The foundation of epigenetic editing is formed by the ability to generate fusion proteins of epigenetic enzymes or their catalytic domains with programmable DNA-binding platforms such as the clustered regularly interspaced short palindromic repeat (e.g., CRISPR Cas9 or TnpB) to target these to an endogenous locus of choice. The enzymatic fusion protein then dictates the initial deposited modification while subsequent cross-talk within the local chromatin environment likely influences epigenetic and transcriptional output.

The following published literature discussing epigenetic editing is incorporated herein by reference each in their entireties.
Gjaltema RAF, Rots M G. Advances of epigenetic editing. Curr Opin Chem Biol. 2020 August;57:75-81. doi: 10.1016/j.cbpa.2020.04.020. Epub 2020 Jun. 30. PMID: 32619853.
Kleinstiver B P, Sousa A A, Walton R T, Tak Y E, Hsu J Y, Clement K, Welch M M, Horng J E, Malagon-Lopez J, Scarfò I, Maus M V, Pinello L, Aryee M J, Joung J K. Engineered CRISPR-Cas12a variants with increased activities and improved targeting ranges for gene, epigenetic and base editing. Nat Biotechnol. 2019 March;37 (3): 276-282. doi: 10.1038/s41587-018-0011-0. Epub 2019 Feb. 11. Erratum in: Nat Biotechnol. 2020 July;38 (7): 901. PMID: 30742127; PMCID: PMC6401248.
Rots M G, Jeltsch A. Editing the Epigenome: Overview, Open Questions, and Directions of Future Development. Methods Mol Biol. 2018; 1767:3-18. doi: 10.1007/978-1-4939-7774-1_1. PMID: 29524127.
Liu X S, Jaenisch R. Editing the Epigenome to Tackle Brain Disorders. Trends Neurosci. 2019 December;42 (12): 861-870. doi: 10.1016/j.tins.2019.10.003. Epub 2019 Nov. 7. PMID: 31706628.
Waryah C B, Moses C, Arooj M, Blancafort P. Zinc Fingers, TALEs, and CRISPR Systems: A Comparison of Tools for Epigenome Editing. Methods Mol Biol. 2018; 1767:19-63. doi: 10.1007/978-1-4939-7774-1 2. PMID: 29524128.
Xu X, Hulshoff M S, Tan X, Zeisberg M, Zeisberg E M. CRISPR/Cas Derivatives as Novel Gene Modulating Tools: Possibilities and In Vivo Applications. Int J Mol Sci. 2020 Apr. 25;21 (9): 3038. doi: 10.3390/ijms21093038. PMID: 32344896; PMCID: PMC7246536.

In addition, the following published patent literature relating to epigenetic editing is incorporated herein by reference each in their entireties.

| Publication | |
|---|---|
| Number | Title |
| WO2023283359A2 | COMPOSITIONS AND |
| | METHODS FOR MOD- |
| | ULATING SECRETED |
| | FRIZZLED RECEPTOR |
| | PROTEIN 1 (SFRP1) |
| | GENE EXPRESSION |
| WO2022226139A1 | TISSUE-SPECIFIC |
| | NUCLEIC ACID |
| | DELIVERY BY MIXED |
| | CATIONIC LIPID |
| | PARTICLES |
| WO2022132926A1 | TISSUE-SPECIFIC |
| | NUCLEIC ACID |
| | DELIVERY BY 1,2- |
| | DIOLEOYL- |
| | 3- |
| | TRIMETHYLAMMONIUM- |
| | PROPANE (DOTAP) |
| | LIPID NANOPARTICLES |
| WO2021183720A1 | COMPOSITIONS AND |
| | METHODS FOR MOD- |
| | ULATING FORKHEAD |
| | BOX P3 (FOXP3) |
| | GENE EXPRESSION |
| WO2021061815A1 | COMPOSITIONS AND |
| | METHODS FOR MOD- |
| | ULATING |
| | HEPATOCYTE |
| | NUCLEAR FACTOR |
| | 4-ALPHA (HNF4α) |
| | GENE EXPRESSION |
| WO2021061707A1 | COMPOSITIONS AND |
| | METHODS FOR MOD- |
| | ULATING APOLIPO- |
| | PROTEIN B (APOB) |
| | GENE EXPRESSION |
| WO2021061698A1 | METHODS AND |
| | COMPOSITIONS |
| | FOR MODULATING |
| | FRATAXIN |
| | EXPRESSION AND |
| | TREATING |
| | FRIEDRICH'S ATAXIA |

### 3. Diseases and Disorders

Provided herein are methods of treating a disease or disorder, the methods comprising administering to a subject in need thereof a pharmaceutical composition of the present disclosure. In various embodiments of the invention, target genome or epigenetic modifications include cells with monogenic diseases or disorders. Various monogenic diseases include but are not limited to: Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylkeotnuria; Severe Combined Immunodeficiency; Sickle Cell Disease; Smith-Lemli-Opitz Syndrome; Tay-Sachs Disease; hereditary tyrosinemia I; Influenza; SARS-CoV-2; Alzheimer's disease; Parkinson's disease.

Target sequences related to certain diseases and disorders are known in some cases. Target sequences or target editing sites include disease-associated or causative mutations for one or more of 10,000 monogenic disorders. A list of target sequences can be generated based on the monogenic disorders. Common genetic disorders that may be correctable by the TnpB gene editing systems described here including but are not limited to: Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylkeotnuria; Severe Combined Immunodeficiency; Sickle Cell Disease; Smith-Lemli-Opitz Syndrome; and Tay-Sachs Disease. In other embodiments, the disease-associated gene can be associated with a polygenic disorder selected from the group consisting of: heart disease; high blood pressure; Alzheimer's disease; arthritis; diabetes; cancer; and obesity.

The TnpB gene editing systems disclosed herein may also be used to treat the following genetic disorders by editing a defect in the disease-associated gene, as follows:

| | Disease |
|---|---|
| Genetic disease | gene |
| Arenoleukodystrophy (ALD) | ABCD1 |
| Agammaglobulinemia non-Bruton type | IGHM |
| Alport syndrome | COL4A5 |
| Amyloid neuropathy - Andrade disease | TTR |
| Angioneurotic oedema | C1NH |
| Alpha1-antitrypsin deficiency | SERPINEA1 |
| Bartter syndrome type 4 | BSND |
| Blepharophimosis - ptosis - epicanthus inversus | FOXL2 |
| syndrome (BEPS) | |
| Brugada sindrome - Long QT syndrome-3 | SCN5A |
| Bruton agammaglobulinemia tyrosine kinase | BTK |
| Ceroid lipofuscinosis neuronal type 2 | CLN2 |
| Charcot Marie Tooth type 1A (CMT1A) | PMP22 |
| Charcot Marie Tooth type X (CMTX) | CMTX |
| Chronic granulomatous disease (CGD) | CYBB |
| Cystic Fibrosis (CF) | CFTR |
| Congenital adrenal hyperplasia (CAH) | CYP21A2 |
| Congenital disorder of glycosylation type Ia (CDG Ia) | PMM2 |
| Congenital fibrosis of extraocular muscles 1 (CFEOM1) | KIF21A |
| Crigler-Najjar syndrome | UGT1A1 |
| Deafness, autosomal recessive | CX26 |
| Diamond-Blackfan anemia (DBA) | RPS19 |
| Duchenne-Becker muscular dystrophy (DMD/DMB) | DMD |
| Duncan disease - X-linked lymphoproliferative | SH2D1A |
| syndrome (XLPD) | |
| Ectrodactyly ectodermal dysplasia and cleft lip/palate | p63 |
| syndrome (EEC) | |
| Epidermolysis bullosa dystrophica/pruriginosa | COL7A1 |
| Exostoses multiple type I (EXT1) | EXT1 |
| Exostoses multiple type II (EXT2) | EXT2 |
| Facioscapulohumeral muscular dystrophy | FRG1 |
| Factor VII deficiency | F7 |
| Familial Mediterranean Fever (FMF) | MEFV |
| Fanconi anemia A | FANCA |
| Fanconi anemia G | FANCG |
| Fragile-X | FRAXA |
| Gangliosidosis (GM1) | GLB1 |
| Gaucher disease (GD) | GBA |
| Glanzmann thrombasthenia | ITGA2B |
| Glucose-6-phosphate dehydrogenase deficiency | G6PD |
| Glutaric acidemia I | GCDH |
| Haemophilia A | F8 |
| Haemophilia B | F9 |
| Hand-foot-uterus syndrome | HOXD13 |
| Hemophagocytic lymphohistiocytosis familial, type 2 | PRF1 |
| (FHL2) | |
| Hypomagnesaemia primary | CLDN16 |
| HYPOPHOSPHATASIA | ALPL |
| Holt-Oram Sindrome (HOS) | TBX5 |
| Homocystinuria | MTHFR |
| Incontinentia pigmenti | NEMO |
| Lesch-Nyhan syndrome | HPRT |
| Limb-girdle muscular dystrophy type 2C (LGMD2C) | SGCG |
| Long QT syndrome-1 | KCNQ1 |
| Mannosidosis Alpha | MAN2B1 |
| Marfan syndrome | FBN1 |
| Methacrylic Aciduria, deficiency of beta- | HIBCH |
| hydroxyisobutyryl-CoA deacylase | |
| Mevalonic aciduria | MVK |
| Myotonic dystrophy (DM) | DMPK |
| Myotonic dystrophy type 2 (DM2) | ZNF9 |
| Mucopolysaccharidosis Type I - Hurler syndrome | IDUA |
| Mucopolysaccharidosis Type IIIA - Sanfilippo sindrome | SGSH |
| A (MPS3A) | |
| Mucopolysaccharidosis Type IIIB - Sanfilippo sindrome | NAGLU |
| B (MPS3B) | |
| Mucopolysaccharidosis Type VI (MPS VI) - Maroteaux- | ARSB |
| Lamy Syndrome | |
| Neuronal ceroid lipofuscinosis 1 - Batten's disease | PPT1 |
| (CLN1) | |
| Niemann-Pick disease | SMPD1 |
| Noonan sindrome | PTPN11 |
| Pancreatitis, hereditary (PCTT) | PRSS1 |
| Paramyotonia congenita (PMC) | SCN4A |
| Phenylketonuria | PAH |
| Polycystic kidney disease type 1 (PKD1) | PKD1 |
| Polycystic kidney disease type 2 (PKD2) | PKD2 |
| Polycystic kidney and hepatic disease-1 (ARPKD) | PKHD1 |
| Schwartz-Jampel/Stuve-Wiedemann syndrome | LIFR |
| Sickle cell anemia | HBB |
| Synpolydactyly (SPD1) | HOXA13 |
| Smith-Lemli-Opitz syndrome | DHCR7 |
| Spastic paraplegia type 3 | SPG3A |
| Spinal Muscular Atrophy (SMA) | SMN |
| Spinocerebellar ataxia 3 (SCA3) | ATXN3 |
| Spinocerebellar ataxia 7 (SCA7) | ATXN7 |
| Stargardt disease | ABCA4 |
| Tay Sachs (TSD) | HEXA |
| Thalassemia-α mental retardation syndrome | ATRX |
| Thalassemia-β | HBB |
| Torsion dystonia, early onset (EOTD) | DYT1 |
| Tyrosinaemia type 1 | FAH |
| Tuberosclerosis 1 | TSC1 |
| Tuberosclerosis 2 | TSC2 |
| Wiskott-Aldrich Sindrome (WAS) | WAS |

In addition, the TnpB gene editing systems disclosed herein may also be used to treat the following genetic disorders by editing a defect in the disease-associated gene, or in more than one gene associated with a particular disorders, as follows:

| | B | C | D | | F |
|---|---|---|---|---|---|
| A | Disease- | Most | Encoded | E | Type of |
| Genetic | associated | common of | product of | Accession | product of |
| disease | genes | (B) | (C) | No. of (C) | (C) |
| Adrenal | CYP21A2 | CYP21A2 | Cytochrome P450 | P08686 | Enzyme |
| hyperplasia | | (196) | family 21 subfamily A | | |
| due to 21- | | | member 2 | | |
| hydroxylase | | | | | |
| deficiency | | | | | |
| (21-OHD | | | | | |
| CAH) | | | | | |
| Aicardi- | ADAR; | RNASEH2B | Ribonuclease H2 | Q5TBB1 | Enzyme |
| Goutières | IFIH1; | (28) | subunit B | | |
| syndrome | RNASEH2A; | | | | |
| encephalopathy | RNASEH2B; | | | | |
| | RNASEH2C; | | | | |
| | SAMHD1; | | | | |
| | TREX1 | | | | |
| Alpha-1- | SERPINA1 | SERPINA1 | Serpin family A | P01009 | Enzyme |
| antitrypsin | | (83) | member 1 | | inhibitor |
| (A1AT) | | | | | |
| deficiency | | | | | |
| (AATD) | | | | | |
| Arrhythmogenic | 13 different | PKP2 (138) | Plakophilin 2 | Q99959 | Adhesion |
| right | genes | | | | protein in |
| ventricular | linked to | | | | junctions and |
| cardiomyopathy/ | this | | | | intermediate |
| dysplasia | disorder, so | | | | filaments. |
| (ARVC, | far. | | | | |
| ARVD) | | | | | |
| Autosomal | BICC1; | PKD1 (1154) | Polycystin 1 | P98161 | Subunit of ion |
| dominant | GANAB; | | | | channel |
| polycystic | PKD1; | | | | complex |
| kidney | PKD2 | | | | |
| disease | | | | | |
| (ADPKD) | | | | | |
| Brugada | 22 different | SCN5A (725) | Sodium voltage-gated | Q14524 | Ion channel |
| syndrome | genes | | channel alpha subunit | | |
| ventricular | linked to | | 5 | | |
| fibrillation | this | | | | |
| | disorder, so | | | | |
| | far | | | | |
| Catecholaminergic | CALM1; | RYR2 (288) | Ryanodine receptor 2 | Q92736 | Ion channel |
| polymorphic | CALM2; | | | | |
| ventricular | CALM3; | | | | |
| tachycardia | CASQ2; | | | | |
| (CPVT) | RYR2; | | | | |
| | TECRL; | | | | |
| | TRDN | | | | |
| Charcot- | 75 different | PMP22 (63) | Peripheral myelin | Q01453 | Ill-defined role |
| Marie- | genes | | protein 22^{d} | | in myelin and |
| Tooth^{d} disease/ | linked to | | | | Schwann cells |
| Hereditary | this | | | | |
| motor and | disorder, so | | | | |
| sensory | far. | | | | |
| neuropathy | | | | | |
| Congenital | CYP11B1; | CYP21A2 | Cytochrome P450 | P08686 | Enzyme |
| adrenal | CYP17A1; | (214) | family 21 subfamily A | | |
| hyperplasia | CYP21A2; | | member 2 | | |
| (CAH) | HSD3B2; | | | | |
| | POR; | | | | |
| | STAR | | | | |
| Congenital | SI | SI (23) | Sucrase-isomaltase | P14410 | Enzyme |
| sucrase- | | | | | |
| isomaltase | | | | | |
| deficiency | | | | | |
| (CSID) | | | | | |
| Congenital | CFTR; | CFTR (120) | Cystic fibrosis | Q20BH0 | Ion channel |
| bilateral | ADGRG2 | | transmembrane | | |
| absence of | | | conductance regulator | | |
| vas deferens | | | | | |
| Cystic | CFTR; | CFTR (1053) | Cystic fibrosis | Q20BH0 | Ion channel |
| fibrosis | CLCA4; | | transmembrane | | |
| | DCTN4; | | conductance regulator | | |
| | STX1A; | | | | |
| | TGFB1 | | | | |
| Cystinuria- | SLC3A1; | SLC7A9 (83) | Solute carrier family 7 | P82251 | Membrane |
| lysinuria | SLC7A9 | | member 9 | | transporter |
| syndrome/ | | | | | |
| Cystinuria | | | | | |
| Cytomegalic | NR0B1 | NR0B1 (112) | Nuclear receptor | P51843 | Nuclear |
| congenital | | | subfamily 0 group B | | receptor |
| adrenal | | | member 1 | | |
| hypoplasia | | | | | |
| (AHC) | | | | | |
| (subtype of | | | | | |
| congenital | | | | | |
| adrenal | | | | | |
| hypoplasia) | | | | | |
| Dentinogenesis | DSPP | DSPP (11) | Dentin sialophospho- | Q9NZW4 | Seeds |
| imperfecta | | | protein | | biomineralization, |
| (DGI) (all | | | | | Dentinogenesis |
| types) | | | | | |
| Duchenne | DMD; | DMD (830) | Dystrophin | P11532 | Structural |
| muscular | LTBP4 | | | | protein |
| dystrophy | | | | | |
| (DMD) | | | | | |
| Dysbetalipoproteinemia/ | APOE | APOE (42) | Apolipoprotein E | P02649 | Lipid carrier, |
| Hyperliproteinemia | | | | | lipoprotein |
| type 3 | | | | | |
| Ehlers- | COL1A1; | COL5A1 | Collagen type V alpha | P20908, | Structural |
| Danlos | COL5A1; | (106) | 1 chain, collagen type | P05997 | protein |
| syndrome | COL5A2 | | V alpha 2 chain | | |
| Familial | APC; | APC (539) | Adenomatous | P25054 | Tumor |
| adenomatous | MUTYH | | polyposis coli protein | | suppressor, |
| polyposis | | | | | regulatory |
| (FAP) | | | | | protein |
| Gardner | APC | APC (539) | Adenomatous | P25054 | Tumor |
| syndrome | | | polyposis coli protein | | suppressor, |
| (subtype of | | | | | associated |
| familial | | | | | with |
| adenomatous | | | | | microtublules |
| polyposis) | | | | | |
| Familial | CCM2; | KRIT1 (80) | Krev interaction | O00522 | Regulatory |
| cerebral | KRIT1; | | trapped protein 1 | | protein |
| cavernous | PDCD10 | | | | |
| malformation | | | | | |
| Familial | CASR | CASR (373) | Calcium sensing | P41180 | G protein- |
| hypocalciuric | | | receptor | | coupled |
| hypercalcemia | | | | | receptor |
| type 1 | | | | | |
| (FHH) | | | | | |
| Famililal | APOB; | LDLR (1254) | Low density | P01130 | Lipoprotein |
| hypercholesterolemia | LDLR; | | lipoprotein receptor | | receptor |
| | LDLRAP1; | | | | |
| | PCSK | | | | |
| Familial | 45 different | TTN (672) | Titin | Q8WZ42 | Muscle protein |
| isolated | genes | | | | |
| dilated | linked to | | | | |
| cardiomyopathy | this | | | | |
| | disorder, so | | | | |
| | far. | | | | |
| Familial long | 19 different | KCNQ1 (448) | Potassium voltage- | P51787 | Ion Channel |
| QT syndrome | genes | | gated channel | | |
| (LQTS), | linked to | | subfamily Q member 1 | | |
| including | this | | | | |
| Romano- | disorder, so | | | | |
| Ward | far. | | | | |
| syndrome | | | | | |
| Fragile X | FMR1 | FMR1 (7) | Fragile X mental | Q06787 | Regulator of |
| syndrome/ | | | retardation 1 | | mRNA |
| Martin-bell | | | | | biology |
| syndrome | | | | | |
| Glucose-6- | G6PD | G6PD (218) | Glucose-6-phosphate 1 | P11413 | Enzyme |
| phosphate | | | dehydrogenase | | |
| dehydrogenase | | | | | |
| deficiency | | | | | |
| Glycogen | 27 different | AGL (117) | Glycogen debranching | P35573 | Enzyme |
| storage | genes | | enzyme | | |
| disease | linked to | | | | |
| | this | | | | |
| | disorder, so | | | | |
| | far. | | | | |
| GM2 | GM2A; | HEXA (124) | Hexosaminidase | P06865 | Enzyme |
| gangliosidosis | HEXA; | | subunit alpha | | |
| | HEXB | | | | |
| Hemochromatosis | BMP6; | HFE (43) | Hereditary | Q30201 | Binds |
| | HAMP; | | hemochromatosis | | transferrin |
| | HFE; HJV; | | protein | | receptor |
| | SLC40A1; | | | | |
| | TFR2 | | | | |
| Hemolytic | PKLR | PKLR (237) | Pyruvate kinase | P30613 | Enzyme |
| anemia due to | | | | | |
| red cell | | | | | |
| pyruvate | | | | | |
| kinase | | | | | |
| deficiency | | | | | |
| Hemophilia | F8; F9 | F8 (1898) | Coagulation factor | P00451 | Cofactor for |
| A and B | | | VIII | | factor IXa |
| Hemophilia | F8 | F8 (3364) | Coagulation factor | P00451 | Cofactor for |
| A | | | VIII | | factor IXa |
| Hemorrhagic | ACVRL1; | ENG (187) | Endoglin | P17813 | Regulation of |
| telangiectasia/ | ENG; | | | | angiogenesis |
| Osler Weder | GDF2; | | | | |
| Rendu | SMAD4 | | | | |
| disease | | | | | |
| Hereditary | ANGPT1; | SERPING1 | Serpin family G | P05155 | Enzyme |
| angioedema | F12; PLG; | (252) | member 1 | | inhibitor |
| (HAE)/ | SERPING1 | | | | |
| Angioneurotic | | | | | |
| edema | | | | | |
| Hereditary | 14 different | BRCA1 | Breast cancer type 1 | P38398 | E3 ubiquitin- |
| breast and | genes | (1262) | susceptibility protein | | protein ligase |
| ovarian | linked to | | | | |
| cancer | this | | | | |
| syndrome | disorder so | | | | |
| | far. | | | | |
| Hereditary | ALDOB | ALDOB (32) | Aldolase, fructose- | P05062 | Enzyme |
| fructose | | | bisphosphate B | | |
| intolerance/ | | | | | |
| Fructosemia | | | | | |
| Hereditary | MOCOS; | MOCOS (8); | Molybdenum cofactor | Q9C5X8, | Enzymes |
| xanthinuria/ | XDH | XDH (17) | sulfurase, xanthine | P47989 | |
| Xanthine | | | dehydrogenase | | |
| stone disease | | | | | |
| Hypohidrotic | 10 different | EDA (199) | Ectodysplasin A | Q92838 | Cytokine |
| ectodermal | genes | | | | |
| dysplasia | linked to | | | | |
| (HED) | this | | | | |
| | disorder, so | | | | |
| | far. | | | | |
| Iminoglycinuria | SLC36A2; | SLC36A2 (1) | Solute carrier family | Q495M3 | Membrane |
| | SLC6A18; | | 36 member 2 | | transporter |
| | SLC6A19; | | | | |
| | SLC6A20 | | | | |
| Li-Fraumeni | CDKN2A; | TP53 (417) | Tumor protein p53 | P04637 | Tumor |
| syndrome | CHEK2; | | | | suppressor, |
| sarcoma, | MDM2; | | | | gene |
| breast, | TP53 | | | | regulation |
| leukemia, | | | | | |
| and adrenal | | | | | |
| gland | | | | | |
| (SBLA) | | | | | |
| syndrome | | | | | |
| Long chain 3- | HADHA | HADHA (35) | Hydroxyacyl-CoA | P40939 | Enzyme |
| hydroxyacyl- | | | dehydrogenase | | |
| CoA | | | trifunctional | | |
| dehydrogenase | | | multienzyme complex | | |
| deficiency | | | subunit alpha | | |
| (LCHAD) | | | | | |
| Lynch | 11 different | MSH2 (34) | DNA mismatch repair | P43246 | DNA repair, |
| syndrome | genes | | protein Msh2 | | binds DNA, |
| | linked to | | | | ATPase |
| | this | | | | |
| | disorder so | | | | |
| | far | | | | |
| Marfan | FBN1; | FBN1 (1893) | Fibrillin 1 | P35555 | Structural |
| syndrome | TGFBR2 | | | | protein, |
| | | | | | extracellular |
| | | | | | matrix |
| Maternal | PAH | PAH (690) | Phenylalanine | P00439 | Enzyme |
| phenylketonuria/ | | | hydroxylase | | |
| Phenylketonuric | | | | | |
| embryopathy | | | | | |
| Medium | ACADM | ACADM | Acyl-CoA | P11310 | Enzyme |
| chain acyl- | | (136) | dehydrogenase | | |
| CoA | | | medium chain | | |
| dehydrogenase | | | | | |
| deficiency | | | | | |
| (MCADD) | | | | | |
| Mucolipidosis | GNPTAB | GNPTAB (68) | N-acetylglucosamine 1 | Q3T906 | Enzyme |
| type III | | | phosphate transferase, | | |
| (ML3) | | | Subunits and | | |
| alpha/beta | | | beta | | |
| Mucopolysaccharidosis | GALNS | GALNS (269) | Galactosamine (N- | P34059 | Enzyme |
| type 4A | | | acetyl)-6-sulfatase | | |
| (MPS4A)/ | | | | | |
| Morquio | | | | | |
| disease | | | | | |
| type A | | | | | |
| Multiple | RET | RET (130) | Ret proto-oncogene | P07949 | Receptor |
| endocrine | | | receptor tyrosine | | tyrosine kinase |
| neoplasia | | | kinase | | |
| type 2 | | | | | |
| Multiple | COL2A1; | COMP (155) | Cartilage oligomeric | P49747 | Structural |
| epiphyseal | COL9A1; | | matrix protein | | protein |
| dysplasia | COL9A2; | | | | |
| (MED) | COL9A3/ | | | | |
| | collagen | | | | |
| | type IX | | | | |
| | alpha 3 | | | | |
| | chain; | | | | |
| | COMP; | | | | |
| | KIF7; | | | | |
| | MATN3; | | | | |
| | SLC26A2 | | | | |
| Neurofibromatosis | NF1 | NF1 (1208) | Neurofibromin 1 | P21359 | Regulator of |
| type 1 | | | | | Ras GTPase |
| (NF1)/Von | | | | | activity |
| Recklinghausen | | | | | |
| disease | | | | | |
| Oculocutaneous | LRMDA; | TYR (352) | Tyrosinase | P14679 | Enzyme |
| albinism | MC1R; | | | | |
| (OCA) | OCA2; | | | | |
| | SLC24A5; | | | | |
| | SLC45A2; | | | | |
| | TYR; | | | | |
| | TYRP1 | | | | |
| Osteogenesis | 15 different | COL1A1 | Collagen type I alpha 1 | P02452, | Structural |
| imperfecta/ | genes | (547); | chain, collagen type I | P08123 | protein |
| brittle bone | linked to | COL1A2 | alpha 2 chain | | |
| disease | this | (466) | | | |
| | disorder, so | | | | |
| | far. | | | | |
| Pendred | FOXI1; | SLC26A4 | Solute carrier family | O43511 | Membrane |
| syndrome | KCNJ10; | (404) | 26 member 4 | | transporter |
| (PDS)/ | SLC26A4 | | | | |
| Deafness with | | | | | |
| goiter | | | | | |
| Phenylketonuria | PAH | PAH (690) | Phenylalanine | P00439 | Enzyme |
| (PKU)/ | | | hydroxylase | | |
| Phenylalanine | | | | | |
| hydroxylase | | | | | |
| deficiency | | | | | |
| (PAH | | | | | |
| deficiency) | | | | | |
| Proximal | NAIP; | SMN1 (47) | Survival motor neuron | Q16637 | RNA splicing |
| spinal | SMN1; | | protein | | |
| muscular | SMN2 | | | | |
| atrophy | | | | | |
| (SMA) | | | | | |
| Retinitis | 82 different | RHO (204) | Rhodopsin | P08100 | G-protein |
| Pigmentosa | genes | | | | coupled |
| (RP) | linked to | | | | receptor |
| | this | | | | |
| | disorder, so | | | | |
| | far. | | | | |
| Recessive X- | STS | STS (28) | Steroid sulfatase | P08842 | Enzyme |
| linked | | | | | |
| ichthyosis | | | | | |
| (XLI) | | | | | |
| Retinoblastoma | NMYC; | RB1 (292) | RB transcriptional | P06400 | Tumor |
| (RB | RB1 | | corepressor 1 | | suppressor, |
| bilateral | | | | | cell cycle |
| (40% of | | | | | regulation |
| cases) and | | | | | |
| unilateral | | | | | |
| (60% of | | | | | |
| cases- | | | | | |
| denovo | | | | | |
| mutation) | | | | | |
| Rett | MECP2 | MECP2 (246) | Methyl-CpG binding | P51608 | Binds to |
| syndrome | | | protein 2 | | methylated |
| | | | | | DNA, gene |
| | | | | | regulation |
| Sickle cell | HBB | HBB (433) | Hemoglobin subunit | P68871 | Oxygen carrier |
| anemia | | | beta | | |
| Sotos | APC2; | NSD1 (228) | Nuclear receptor | Q96L73 | Enzyme |
| syndrome/ | NSD1; | | binding SET domain | | |
| cerebral | SETD2 | | protein 1 | | |
| gigantism | | | | | |
| Stargardt | ABCA4; | ABCA4 (789) | ATP binding cassette | P78363 | Membrane |
| disease/Fundus | CNGB3; | | subfamily A member 4 | | transporter |
| flavimaculatus | ELOVL4; | | | | |
| | PROM1; | | | | |
| | PRPH2 | | | | |
| Stickler | COL11A1; | COL2A1 | Collagen type II alpha | P02458 | Structural |
| syndrome/ | COL2A1; | (335) | 1 chain | | protein |
| hereditary | COL11A2; | | | | |
| progressive | COL9A1; | | | | |
| arthroophthalmopathy | COL9A2; | | | | |
| | COL9A3; | | | | |
| | LOXL3 | | | | |
| Supravalvular | ELN | ELN (25) | Elastin | P15502 | Structural |
| aortic | | | | | protien |
| stenosis | | | | | |
| (SVAS) | | | | | |
| β- | HBB | HBB (434) | Hemoglobin B chain | P68871 | Oxygen carrier |
| Thalassemia | | | | | |
| Tibial | TTN | TTN (53) | Titin | Q8WZ42 | Muscle protein |
| muscular | | | | | |
| dystrophy/ | | | | | |
| Upp myopathy | | | | | |
| Tuberous | TSC1; | TSC2 (518) | Tuberin | P49815 | Tumor |
| sclerosis | TSC2 | | | | suppressor, |
| complex/ | | | | | Regulation of |
| Bourneville | | | | | mTORC1 |
| syndrome | | | | | signaling |
| Von-Hippel | VHL | VHL (218) | Von Hippel-Lindau | P40337 | Tumor |
| Lindau | | | tumor suppressor | | suppressor, |
| disease | | | | | role in E3 |
| | | | | | ubiquitin |
| | | | | | ligase complex |
| Von | VWF | VWF (636) | Von Willebrand factor | P04275 | Collagen |
| Willebrand | | | | | binding, |
| disease | | | | | chaperone for |
| | | | | | coagulation |
| | | | | | factor VIII |
| X-linked | ABCD1 | ABCD1 (425) | ATP binding cassette | P33897 | Membrane |
| adrenoleukodystrophy | | | subfamily D member 1 | | transporter |
| (ALD) | | | | | |
| X-linked | RS1 | | | | |
| retinoschisis | | | | | |
| (XLRS) | | | | | |

Accordingly, to treat one or more such diseases or disorders, in various aspects of the invention, one or more targeted polynucleotide sequence related to certain diseases and disorders, e.g., a genetic mutation, is contacted by a TnpB gene editing system disclosed herein; and a guide RNA, wherein the guide RNA comprises a complementary sequence to that of a targeted polynucleotide sequence.

In some embodiments, the guide RNA directs the TnpB polypeptide to the target site or the targeted polynucleotide sequence; and optionally forms a ribonucleoprotein complex with the polypeptide and the guide RNA.

Additional therapeutic applications for the TnpB genome editing systems disclosed herein include base editing, prime editing, gene insertions and/or deletions.

Diagnostic applications for the TnpB genome editing system include probes, diagnostics, theranostics.

The TnpB editing system comprising the heterologous nucleic acid sequence can be used in a variety of applications, several non-limiting examples of which are described herein. In general, the TnpB editing system can be used in any suitable organism. In some embodiments, the organism is a eukaryote.

In some embodiments, the organism is an animal. In some embodiments, the animal is a fish, an amphibian, a reptile, a mammal, or a bird. In some embodiments, the animal is a farm animal or agriculture animal. Non-limiting examples of farm and agriculture animals include horses, goats, sheep, swine, cattle, llamas, alpacas, and birds, e.g., chickens, turkeys, ducks, and geese. In some embodiments, the animal is a non-human primate, e.g., baboons, capuchin monkeys, chimpanzees, lemurs, macaques, marmosets, tamarins, spider monkeys, squirrel monkeys, and vervet monkeys. In some embodiments, the animal is a pet. Non-limiting examples of pets include dogs, cats, horses, rabbits, ferrets, gerbils, hamsters, chinchillas, fancy rats, guinea pigs, canaries, parakeets, and parrots.

In some embodiments, the organism is a plant. Plants that may be transfected with an TnpB editing system include monocots and dicots. Particular examples include, but are not limited to, corn (maize), sorghum, wheat, sunflower, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers. Vegetables include, but are not limited to, crucifers, peppers, tomatoes, lettuce, green beans, lima beans, peas, and members of the genus Cucumis such as cucumber, cantaloupe, and musk melon. Ornamentals include, but are not limited to, azalea, hydrangea, hibiscus, roses, tulips, daffodils, petunias, carnation, poinsettia, and chrysanthemum.

In some embodiments, heterologous nucleic acid sequences can be added to the subject TnpB editing system to provide a cell with a heterologous nucleic acid encoding a protein or regulatory RNA of interest, a cellular barcode, a donor polynucleotide suitable for use in gene editing, e.g., by homology directed repair (HDR) or recombination-mediated genetic engineering (recombineering), or a protospacer DNA sequence for use in molecular recording, as discussed further below. In embodiments relating to TnpB retron-based gene editing systems, uch heterologous sequences may be inserted, for example, into the msr locus or the msd locus such that the heterologous sequence is transcribed by the retron reverse transcriptase as part of the msDNA product.

In some embodiments, the TnpB editing systems described herein may be used for research tools, such as kits, functional genomics assays, and generating engineered cell lines and animal models for research and drug screening. The kit may comprise one or more reagents in addition to the TnpB editing system, such as a buffer, a control reagent, a control vector, a control RNA polynucleotide, a reagent for in vitro production of the polypeptide from DNA, and adaptors for sequencing. A buffer can be, for example, a stabilization buffer, a reconstituting buffer, a diluting buffer, a wash buffer, or a buffer for introducing a polypeptide and/or polynucleotide of the kit into a cell. In some instances, a kit can comprise one or more additional reagents specific for plants. One or more additional reagents for plants can include, for example, soil, nutrients, plants, seeds, spores, Agrobacterium, a T-DNA vector, and a pBINAR vector.

### 4. Production of Protein or RNA

In some embodiments, the TnpB gene editing systems may comprise one or more additional proteins (e.g., an accessory protein, such as a recombinase) or RNA molecules (e.g., a donor template), or a nucleotide sequence encoding the one or more additional proteins or RNA molecules.

In some embodiments, TnpB gene editing systems may comprise a nucleic acid molecule encoding a polypeptide of interest. The polypeptide of interest may be any type of protein/peptide including, without limitation, an enzyme, an extracellular matrix protein, a receptor, transporter, ion channel, or other membrane protein, a hormone, a neuropeptide, an antibody, or a cytoskeletal protein, a functional fragment thereof, or a biologically active domain of interest. In some embodiments, the protein is a therapeutic protein, therapeutic antibody for use in treatment of a disease, or a template to fix a mutation or mutated exon in the genome. In other embodiments, the polypeptide of interest is a gene editing accessory protein, e.g., recombinases, invertases, nucleases, polymerases, ligases, deaminases, reverse transcriptases, or epigenetic modifying functions. The polypeptide of interest, e.g., recombinases, invertases, nucleases, polymerases, ligases, deaminases, reverse transcriptases, or epigenetic modifying functions, could be fused to the TnpB gene editing system or a component thereof (e.g., fused to the TnpB nuclease).

In other embodiments, the TnpB gene editing system could also be engineered to include a DNA template.

In still other embodiments, the TnpB gene editing system could also include a least one additional nucleic acid molecule for modulating a target in the cell, e.g., without limitation, a RNA interference (RNAi) nucleic acid or regulatory RNA such as, but not limited to, a microRNA (miRNA), a small interfering RNA (siRNA), a short hairpin RNA (shRNA), a small nuclear RNA (snRNA), a long non-coding RNA (lncRNA), an antisense nucleic acid, and the like.

### 5. Recombineering

Recombineering (recombination-mediated genetic engineering) can be used in modifying chromosomal as well as episomal replicons in cells, for example, to create gene replacements, gene knockouts, deletions, insertions, inversions, or point mutations. Recombineering can also be used to modify a plasmid or bacterial artificial chromosome (BAC), for example, to clone a gene or insert markers or tags.

The TnpB editing systems described herein can be used in recombineering applications to provide linear single-stranded or double-stranded DNA for recombination. Homologous recombination may be mediated by bacteriophage proteins such as RecE/RecT from Rac prophage or Redobd from bacteriophage lambda. The linear DNA should have sufficient homology at 5′ and 3′ ends to a target DNA molecule present in a cell (e.g., plasmid, BAC, or chromosome) to allow recombination.

The linear double-stranded or single-stranded DNA molecule used in recombineering (i.e. donor polynucleotide) comprises a sequence having the intended edit to be inserted flanked by two homology arms that target the linear DNA molecule to a target site for homologous recombination. Homology arms for recombineering typically range in length from 13-300 nucleotides, or 20 to 200 nucleotides, including any length within this range such as 13, 14, 15, 16, 17, 18, 19, 20, 22, 24, 26, 28, 30, 32, 34, 36, 38, 40, 42, 44, 46, 48, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 nucleotides in length. In some embodiments, a homology arm is at least 15, at least 20, at least 30, at least 40, or at least 50 or more nucleotides in length. Homology arms ranging from 40-50 nucleotides in length generally have sufficient targeting efficiency for recombination; however, longer homology arms ranging from 150 to 200 bases or more may further improve targeting efficiency. In some embodiments, 5′ homology arm and 3′ homology arm differ in length. For example, the linear DNA may have about 50 bases at the 5′ end and about 20 bases at the 3′ end with homology to the region to be targeted.

The bacteriophage homologous recombination proteins can be provided to a cell as proteins or by one or more vectors encoding the recombination proteins, such as the vector or vector system. In some embodiments, one or more vectors encoding the bacteriophage recombination proteins are included in the vector system comprising the TnpB editing system msr gene, msd gene, and/or ret gene sequences. Additionally, a number of bacterial strains containing prophage recombination systems are available for recombineering, including, without limitation, DY380, containing a defective 1 prophage with recombination proteins exo, bet, and gam; EL250, derived from DY380, which in addition to the recombination genes found in DY380, also contains a tightly controlled arabinose-inducible flpe gene (flpe mediates recombination between two identical frt sites); EL350, also derived from DY380, which in addition to the recombination genes found in DY380, also contains a tightly controlled arabinose-inducible ere gene (ere mediates recombination between two identical loxP sites; SW102, derived from DY380, which is designed for BAC recombineering using a galK positive/negative selection; SW105, derived from EL250, which can also be used for galK positive/negative selection, but like EL250, contain an ara-inducible Flpe gene; and SW106, derived from EL350, which can be used for galK positive/negative selection, but like EL350, contains an ara-inducible Cre gene. Recombineering can be carried out by transfecting bacterial cells of such strains with an TnpB editing system comprising a heterologous sequence encoding a linear DNA suitable for recombineering. For a discussion of recombineering systems and protocols, see, e.g., Sharan et al. (2009)Nat Protoc. 4 (2): 206-223, Zhang et al. (1998)Nature Genetics 20:123-128, Muyrers et al. (1999)Nucleic Acids Res. 27:1555-1557, Yu et al. (2000) Proc. Natl. Acad. Sci U.S.A. 97 (11): 5978-5983; herein incorporated by reference.

### 6. Molecular Recording

In some embodiments, the TnpB editing system comprises a synthetic protospacer DNA sequence to allow molecular recording. The endogenous CRISPR Cas1-Cas2 system is normally utilized by bacteria and archaea to keep track of foreign DNA sequences originating from viral infections by storing short sequences (i.e., protospacers) that confer sequence-specific resistance to invading viral nucleic acids within genome-based arrays. These arrays not only preserve the spacer sequences but also record the order in which the sequences are acquired, generating a temporal record of acquisition events.

This system can be adapted to record arbitrary DNA sequences into a genomic CRISPR array in the form of “synthetic protospacers” that are introduced into cells using TnpB editing systems. TnpB editing systems carrying the protospacer sequences can be used for integration of synthetic CRISPR protospacer sequences at a specific genomic locus by utilizing the CRISPR system Cas1-Cas2 complex. Molecular recording can be used to keep track of certain biological events by producing a stable genetic memory tracking code. See, e.g., Shipman et al. (2016) Science 353 (6298): aafl 175 and International Patent Application Publication No. WO/2018/191525; herein incorporated by reference in their entireties.

In some embodiments, the CRISPR-Cas system is harnessed to record specific and arbitrary DNA sequences into a bacterial genome. The DNA sequences can be produced by an TnpB editing system within the cell. For example, the TnpB editing system can be used to produce the protospacers within the cell, which are inserted into a CRISPR array within the cell. The cell may be modified to include one or more engineered returns (or vector systems encoding them) that can produce one or more synthetic protospacers in the cell, wherein the synthetic protospacers are added to the CRISPR array. A record of defined sequences, recorded over many days, and in multiple modalities can be generated.

In some embodiments, a single stranded DNA produced in vivo from a first TnpB editing system may be hybridized with a complementary single-stranded DNA produced in vivo from a second TnpB editing system or may form a hairpin structure and then used as a protospacer sequence to be inserted into a CRISPR array as a spacer sequence. The TnpB editing system(s) should provide sufficient levels of the protospacer sequence within a cell for incorporation into the CRISPR array. The use of protospacers generated within the cell extends the in vivo molecular recording system from only capturing information known to a user, to capturing biological or environmental information that may be previously unknown to a user. For example, an msDNA protospacer sequence in an TnpB editing system construct may be driven by a promoter that is downstream of a sensor pathway for a biological phenomenon or environmental toxin. The capture and storage of the protospacer sequence in the CRISPR array records the event. If multiple msDNA protospacers are driven by different promoters, the activity of those promoters is recorded (along with anything that may be upstream of the promoters) as well as the relative order of promoter activity (based on the relative position of spacer sequences in the CRISPR array). At any point after the recording has taken place, the CRISPR array may be sequenced to determine whether a given biological or environmental event has taken place and the order of multiple events, given by the presence and relative position of msDNA-derived spacers in the CRISPR array.

In some embodiments, the synthetic protospacer further comprises an AAG PAM sequence at its 5′ end. Protospacers including 5′ AAG PAM are acquired by the CRISPR array with greater efficiency than those that do not include a PAM sequence.

In some embodiments, Cas1 and Cas2 are provided by a vector that expresses the Cas1 and Cas2 at a level sufficient to allow the synthetic protospacer sequences produced by TnpB editing systems to be acquired by a CRISPR array in a cell. Such a vector system can be used to allow molecular recording in a cell that lacks endogenous Cas proteins.

### 7. Therapeutic Applications

Also provided herein are methods of diagnosing, prognosing, treating, and/or preventing a disease, state, or condition in or of a subject, using the TnpB editing system of the invention.

Generally, the methods of diagnosing, prognosing, treating, and/or preventing a disease, state, or condition in or of a subject can include modifying a polynucleotide in a subject or cell thereof using a composition, system, or component thereof of the TnpB editing system as described herein, and/or include detecting a diseased or healthy polynucleotide in a subject or cell thereof using a composition, system, or component thereof of the TnpB editing system as described herein.

In some embodiments, the method of treatment or prevention can include using a composition, system, or component of the TnpB editing system to modify a polynucleotide of an infectious organism (e.g. bacterial or virus) within a subject or cell thereof.

In some embodiments, the method of treatment or prevention can include using a composition, system, or component of the TnpB editing system to modify a polynucleotide of an infectious organism or symbiotic organism within a subject.

In some embodiments, the composition, system, and components of the TnpB editing system can be used to develop models of diseases, states, or conditions.

In some embodiments, the composition, system, and components of the TnpB editing system can be used to detect a disease state or correction thereof, such as by a method of treatment or prevention described herein.

In some embodiments, the composition, system, and components of the TnpB editing system can be used to screen and select cells that can be used, for example, as treatments or preventions described herein.

In some embodiments, the composition, system, and components thereof can be used to develop biologically active agents that can be used to modify one or more biologic functions or activities in a subject or a cell thereof.

In general, the method can include delivering a composition, system, and/or component of the TnpB editing system to a subject or cell thereof, or to an infectious or symbiotic organism by a suitable delivery technique and/or composition. Once administered, the components can operate as described elsewhere herein to elicit a nucleic acid modification event. In some embodiments, the nucleic acid modification event can occur at the genomic, epigenomic, and/or transcriptomic level. DNA and/or RNA cleavage, gene activation, and/or gene deactivation can occur.

The composition, system, and components of the TnpB editing system as described elsewhere herein can be used to treat and/or prevent a disease, such as a genetic and/or epigenetic disease, in a subject; to treat and/or prevent genetic infectious diseases in a subject, such as bacterial infections, viral infections, fungal infections, parasite infections, and combinations thereof; to modify the composition or profile of a microbiome in a subject, which can in turn modify the health status of the subject; to modify cells ex vivo, which can then be administered to the subject whereby the modified cells can treat or prevent a disease or symptom thereof; or to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein of the TnpB editing system, and administering them to the subject.

Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component of the TnpB editing system, and comprising multiple Cas effectors.

Also provided is a method of treating a subject, e.g., a subject in need thereof, comprising inducing gene editing by transforming the subject with the Cas effector(s), and encoding and expressing in vivo the remaining portions of the composition, system, (e.g., RNA, guides), complex or component of the TnpB editing system. A suitable repair template may also be provided by the TnpB editing system as described herein elsewhere.

Also provided is a method of treating a subject, e.g., a subject in need thereof, comprising inducing transcriptional activation or repression by transforming the subject with the systems or compositions herein.

Also provided is a method of inducing one or more polynucleotide modifications in a eukaryotic or prokaryotic cell or component thereof (e.g. a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In some embodiments, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiment or the cell of any one of the above embodiment.

Also provided herein is the use of any of the above delivery systems, e.g., LNP delivery system in ex vivo or in vivo gene or genome editing; or for use in in vitro, ex vivo or in vivo gene editing.

Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In some embodiments, target polynucleotide modification using the subject TnpB editing system and the associated compositions, vectors, systems and methods comprise addition, deletion, or substitution of 1 nucleotide to about 10,000 nucleotides at each target sequence of said polynucleotide of said cell(s). The modification can include the addition, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, 75, 100, 200, 250, 300, 500, 1000, 1500, 2000, 2500, 3000, 3500, 4000, 5000, 6000, 7000, 8000, 9000, 10,000 or more nucleotides at each target sequence.

In some embodiments, formation of system or complex results in cleavage, nicking, and/or another modification of one or both strands in or near (e.g. within 1, 2, 3, 4, 5, 6, 7, 8, 9, 10, 20, 50, or more base pairs from) the target sequence.

In some embodiments, a method of modifying a target polynucleotide in a cell to treat or prevent a disease can include allowing a composition, system, or component of the subject TnpB editing system to bind to the target polynucleotide, e.g., to effect cleavage, nicking, or other modification as the composition, system, is capable of said target polynucleotide, thereby modifying the target polynucleotide, wherein the composition, system, or component thereof, complex with a guide sequence, and hybridize said guide sequence to a target sequence within the target polynucleotide, wherein said guide sequence is optionally linked to a tracr mate sequence, which in turn can hybridize to a tracr sequence. In some embodiments, modification can include cleaving or nicking one or two strands at the location of the target sequence by one or more components of the composition, system, or component thereof.

In some embodiments, the TnpB editing system and the associated compositions, systems, vectors, uses, and methods of use, can be used to treat diseases of the circulatory system. In some embodiments, the treatment can be carried out by using an AAV or a lentiviral vector to deliver the TnpB editing system, composition, system, and/or vector described herein to modify hematopoietic stem cells (HSCs) or iPSCs in vivo or ex vivo. In some embodiments, the treatment can be carried out by correcting HSCs or iPSCs as to the disease using a composition, system, herein or a component thereof, wherein the composition, system, optionally includes a suitable HDR repair template (e.g., a template in the msDNA of the TnpB editing system).

In some embodiments, the treatment or prevention for treating a circulatory system or blood disease can include modifying a human cord blood cell. In some embodiments, the treatment or prevention for treating a circulatory system or blood disease can include modifying a granulocyte colony-stimulating factor-mobilized peripheral blood cell (mPB) with any modification described herein. In some embodiments, the human cord blood cell or mPB can be CD34+. In some embodiments, the cord blood cells or mPB cells modified are autologous. In some embodiments, the cord blood cells or mPB cells are allogenic. In addition to the modification of the disease genes, allogenic cells can be further modified using the composition, system, described herein to reduce the immunogenicity of the cells when delivered to the recipient. The modified cord blood cells or mPB cells can be optionally expanded in vitro. The modified cord blood cell(s) or mPB cells can be derived to a subject in need thereof using any suitable delivery technique.

The composition and system may be engineered to target genetic locus or loci in HSCs. In some embodiments, the components of the systems can be codon-optimized for a eukaryotic cell and especially a mammalian cell, e.g., a human cell, for instance, HSC, or iPSC and sgRNA targeting a locus or loci in HSC, such as circulatory disease, can be prepared. These may be delivered via particles, such as the lipid nanoparticle delivery system described herein. The particles may be formed by the components of the systems herein being admixed.

In some embodiments, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16;121 (20): 4082-9. doi: 10.1182/blood-2012-09-455204. Epub 2013 Mar. 21.

In some embodiments, the HSCs or iPSCs modified are autologous. In some embodiments, the HSCs or iPSCs are allogenic. In addition to the modification of the disease genes, allogenic cells can be further modified using the composition, system, described herein to reduce the immunogenicity of the cells when delivered to the recipient.

In some embodiments, the TnpB editing system and the associated compositions, systems, vectors, uses, and methods of use, can be used to treat neurological diseases. In some embodiments, the neurological diseases comprise diseases of the brain and CNS.

Delivery options for the diseases in the brain include encapsulation of the systems in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors or vector systems of the invention. In other embodiments, an artificial virus can be generated for CNS and/or brain delivery.

In some embodiments, the TnpB editing system and the associated compositions, systems, vectors, uses, and methods of use, can be used to treat hearing diseases or hearing loss in one or both ears. Deafness is often caused by lost or damaged hair cells that cannot relay signals to auditory neurons. In some embodiments, the composition, system, or modified cells can be delivered to one or both ears for treating or preventing hearing disease or loss by any suitable method or technique known in the art, such as US20120328580 (e.g., auricular administration), by intratympanic injection (e.g., into the middle ear), and/or injections into the outer, middle, and/or inner ear; administration in situ, via a catheter or pump (U.S. 2006/0030837) and Jacobsen (U.S. Pat. No. 7,206,639). Also see US20120328580. Cells resulting from such methods can then be transplanted or implanted into a patient in need of such treatment.

In some embodiments, the TnpB editing system and the associated compositions, systems, vectors, uses, and methods of use, can be used to treat diseases in non-dividing cells. Exemplary non-dividing cells include muscle cells or neurons. In such cells, homologous recombination (HR) is generally suppressed in the Gl cell-cycle phase, but can be turned back on using art-recognized methods, such as Orthwein et al. (Nature. 2015 Dec. 17; 528 (7582): 422-426).

In some embodiments, the TnpB editing system and the associated compositions, systems, vectors, uses, and methods of use, can be used to treat diseases of the eye.

In some embodiments, the TnpB editing system and the associated compositions, systems, vectors, uses, and methods of use, can be used to treat muscle diseases and cardiovascular diseases.

In some embodiments, the TnpB editing system and the associated compositions, systems, vectors, uses, and methods of use, can be used to treat diseases of the liver and kidney.

In some embodiments, the TnpB editing system and the associated compositions, systems, vectors, uses, and methods of use, can be used to treat epithelial and lung diseases.

In some embodiments, the TnpB editing system and the associated compositions, systems, vectors, uses, and methods of use, can be used to treat diseases of the skin.

In some embodiments, the TnpB editing system and the associated compositions, systems, vectors, uses, and methods of use, can be used to treat cancer.

In some embodiments, the TnpB editing system and the associated compositions, systems, vectors, uses, and methods of use, can be used in adoptive cell therapy.

In some embodiments, the TnpB editing system and the associated compositions, systems, vectors, uses, and methods of use, can be used to treat infectious diseases.

In some embodiments, the TnpB editing system and the associated compositions, systems, vectors, uses, and methods of use, can be used to treat mitochondrial diseases.

In some embodiments, the TnpB editing system and the associated compositions, systems, vectors, uses, and methods of use, can be used to treat hemoglobinopathies. The hemoglobinopathies are a group of disorders passed down through families in which there is abnormal production or structure of the hemoglobin molecule. Sickle cell disease (SCD) is one such blood disorder caused by the abnormal hemoglobin that damages and deforms red blood cells. The abnormal red cells break down, causing anemia, and obstruct blood vessels, leading to recurrent episodes of severe pain and multi-organ ischemic damage. SCD affects millions of people throughout the world and is particularly common among people whose ancestors come from sub-Saharan Africa, regions in the Western Hemisphere (South America, the Caribbean, and Central America); Saudi Arabia; India; and Mediterranean countries such as Turkey, Greece and Italy. There is no widely available cure for SCD although some children have been successfully treated with blood stem cell, or bone marrow, transplants. However, hematopoietic stem cell transplant is not widely done for SCD, because of the difficulty in finding a matched donor. Therefore, the number of people with SCD who get transplants is low. In addition, there are several complications associated with the procedure, including death in about 5 percent of people. In SCD, clinical severity varies, ranging from mild and sometimes asymptomatic states to severe symptoms requiring hospitalization. Symptomatic treatments exist, and newborn screening (NBS) for SCD can reduce the burden of the disease on affected newborns and children.

Thalassemia is another type of blood disorder that is caused by a defect in the gene that helps control the production of the globin chains that make up the hemoglobin molecule. There are two main types of thalassemia: (a) Alpha thalassemia occurs when a gene or genes related to the alpha globin protein are missing or changed (mutated). Alpha thalassemias occur most often in persons from Southeast Asia, the Middle East, China and in those of African descent. (b) Beta thalassemia occurs when a beta globin gene is changed (mutated) so as to affect production of the beta globin protein. Beta thalassemias occur most often in persons of Mediterranean origin. To a lesser extent, Chinese, other Asians and African Americans can be affected.

The TnpB editing system may be used to target a correction in the defective gene that causes the hemoglobinopathy.

All publications, published patent documents, and patent applications cited herein are hereby incorporated by reference to the same extent as though each individual publication, published patent document, or patent application was specifically and individually indicated as being incorporated by reference.

### E. Host Cells

One aspect of the disclosure provides an isolated host cell that includes one or more of the compositions described herein, including, but not limited to, a TnpB gene editing system or any component thereof. In some embodiments, the host cell is a prokaryotic cell, an archaeal cell, or a eukaryotic host cell. In some embodiments, the eukaryotic host cell is a mammalian cell, such as a human cell, a non-human cell, or a non-human mammalian cell. In some embodiments, the host cell is an artificial cell or genetically modified cell. In some embodiments, the host cell is in vitro, such as a tissue culture cell. In some embodiments, the host cell is within a living host organism.

Cells that may contain any of the compositions described herein. The methods described herein are used to deliver a TnpB gene editing system described herein into a eukaryotic cell (e.g., a mammalian cell, such as a human cell). In some embodiments, the cell is in vitro (e.g., cultured cell. In some embodiments, the cell is in vivo (e.g., in a subject such as a human subject). In some embodiments, the cell is ex vivo (e.g., isolated from a subject and may be administered back to the same or a different subject).

The present disclosure contemplates the use of any suitable host cell. For example, the cell host can be a mammalian cell. Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, the cells can be human embryonic kidney (HEK) cells (e.g., HEK 293 or HEK 293T cells). In some embodiments, the cells can be stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cells express stem cell markers and are capable of generating cells characteristic of all three germ layers (ectoderm, endoderm, mesoderm).

Some aspects of this disclosure provide cells comprising any of the compositions disclosed herein, including, but not limited to, TnpB gene editing systems and components and vector or vector systems encoding the engineered gene editing systems, and any combinations thereof. In some embodiments, a host cell is transiently or non-transiently transfected with one or more delivery systems described herein, including virus-based systems, virus-like particle systems, and non-virus-base delivery, including LNPs and liposomes. In some embodiments, a cell is transfected as it naturally occurs in a subject. In some embodiments, a cell that is transfected is taken from a subject, i.e., ex vivo transfection. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC)(Manassas, Va.)).

It is an object of the invention to deliver the herein described genome editing system into various host cells. Preferably, each of the components of the genome editing system are delivered together. In other embodiments, one or more of the components of the genome editing system are delivered separately. In some embodiments, the gene editing components are delivered as DNA molecule, RNA molecules, proteins, nucleoproteins, or combinations thereof.

Alternatively, provided also are delivery of the genome editing system using plasmids.

Suitable host cell is selected from one or more prokaryotic cells, mammalian cells, human cells or synthetic cells. Various tissue types are selected based on the delivery modality. In various embodiments, the various host cells transformed, transduced or the uptake of the genome editing system produces a site-specific modification of a targeted polynucleotide sequence of a host cell genome.

Exemplary host cells for the methods and compositions of the invention include but are not limited to prokaryotic cells, yeast or fungal cells, archaea cells, plant cells, animal cells or human cells.

In various other aspects, provided are fusion protein comprising an isolated polypeptide encoded by an isolated or recombinant nucleic acid sequence fused to a heterologous amino acid sequence. Preferably, the fusion protein comprises a nuclease-deficient polypeptide.

In preferred aspects, the TnpB gene editing systems described herein rely on the cells' DNA repair pathways. DNA double-stranded breaks (DSBs) are repaired in cells via the error-prone non-homologous end-joining (NHEJ), or the error-free homologous recombination (HR), the most common form of homology-directed repair (HDR). The DSB repair through NHEJ creates small insertions or deletions (indels), while HDR requires a repair template, which could be a sister chromatid, another homologous region, or an exogenous repair donor. Preferably, the double-stranded breaks (DSBs) created by the TnpB nuclease makes deletions or insertions at a precise loci in the host cell genome. Accordingly, in some embodiments, the method of modifying a targeted polynucleotide sequence comprises homology-directed repair (HDR). In other embodiments, use of the TnpB complex for HDR provides an efficiency of HDR of 1, 1.5, 2, 2.5, 3, 3.5, 4, 4.5, 5, 5.5, 6, 6.5, 7, 7.5, 8, 8.5, 9, 9.5, 10 or higher-fold improvement.

In some cases, the method of modifying a targeted polynucleotide sequence comprises non-homologous end joining (NHEJ). In certain cases, use of the TnpB complex for NHEJ provides an efficiency of 1, 1.5, 2, 2.5, 3, 3.5, 4, 4.5, 5, 5.5, 6, 6.5, 7, 7.5, 8, 8.5, 9, 9.5, 10 or higher-fold improvement. In another aspect of the present invention, host cells transformed with the nucleic acid molecules or vectors of the present invention, and descendants thereof, are provided. In some embodiments of the present invention, these cells carry the nucleic acid sequences of the present invention on vectors, which may but need not be freely replicating vectors. In other embodiments of the present invention, the nucleic acids have been integrated into the genome of the host cells.

In an alternative embodiment, the host cells of the present invention can be mutated by recombination with a disruption, deletion or mutation of the isolated nucleic acid of the present invention so that the activity of one or more enzyme(s) in the host cell is reduced or eliminated compared to a host cell lacking the mutation.

In various aspects of the invention are provided for a modified host cell comprising a synthetic construct comprising: an engineered TnpB protein operably fused to one or more nucleic acid encoding a) an endonuclease; b) a deaminase; c) a reverse transcriptase; d) a transcriptional modulating polypeptide; or e) any combination of a, b, c and/or d.

In more preferred embodiments, the host cell comprises at least two desired modification sequences for multiplexing. For instance, a second donor nucleic acid sequence paired with a second sgRNA, gRNA or reRNA to modify the second target region of the host cell genome. Accordingly, a plurality of donor nucleic acid sequence paired with the respective sgRNA, gRNA or reRNA is used to modify a number of target regions. A plurality of desired modification sequence is used to transform the host cell as described above.

### F. Pharmaceutical Compositions

The present disclosure relates to pharmaceutical compositions comprising TnpB editing systems. In some embodiments, the TnpB editing system comprising one or more polypeptides and cognate guide RNA are formulated as part of a lipid nanoparticle. In some embodiments, a lipid nanoparticle comprises an ionizable lipid, a structural lipid, a PEGylated lipid, and a phospholipid.

In various aspects of the invention, the TnpB genome editing system is delivered as polynucleotides. For instance, in one embodiment, the TnpB nuclease and the reRNA are delivered as polynucleotides and encoded by one or more plasmids (Lauritsen, I., Porse, A., Sommer, M. O. A. et al. A versatile one-step CRISPR-Cas9 based approach to plasmid-curing. Microb Cell Fact 16, 135 (2017); Wasels, François et al. “A two-plasmid inducible CRISPR/Cas9 genome editing tool for Clostridium acetobutylicum.” Journal of microbiological methods vol. 140 (2017): 5-11) doi: 10.1016/j.mimet.2017.06.010). In other embodiments, the TnpB nuclease is encoded in a mRNA and the gRNA is encoded as an in vitro transcribed synthetic oligonucleotide (Yang, Hui et al. “One-step generation of mice carrying reporter and conditional alleles by CRISPR/Cas-mediated genome engineering.” Cell vol. 154,6 (2013): 1370-9. doi: 10.1016/j.cell.2013.08.022). In other aspects, the TnpB nuclease protein and a synthetic gRNA oligonucleotide (Suresh, Bharathi et al. “Cell-Penetrating Peptide-Mediated Delivery of Cas9 Protein and Guide RNA for Genome Editing.” Methods in molecular biology (Clifton, N.J.) vol. 1507 (2017): 81-94. doi: 10.1007/978-1-4939-6518-2_7) or alternatively as an TnpB nuclease protein gRNA RNP complex (Gasiunas, Giedrius et al. “Cas9-crRNA ribonucleoprotein complex mediates specific DNA cleavage for adaptive immunity in bacteria.” Proceedings of the National Academy of Sciences of the United States of America vol. 109,39 (2012): E2579-86. doi: 10.1073/pnas. 1208507109).

The pharmaceutical compositions described herein (e.g., LNP compositions comprising a TnpB gene editing system or components thereof) may be delivered as described in PCT Publication WO2012135805, which is incorporated herein by reference in its entirety, or by another method known or described herein.

In various aspects, the present disclosure provides methods comprising administering a pharmaceutical composition (e.g., LNP formulation comprising a TnpB gene editing system) to a subject in need thereof. The pharmaceutical composition may be administered to a subject using any amount and any route of administration which may be effective for preventing, treating, diagnosing, or imaging a disease, disorder, and/or condition. The exact amount required will vary from subject to subject, depending on factors such as, but not limited to, the species, age, and general condition of the subject, the severity of the disease, the particular composition, its mode of administration, its mode of activity, and the like. The pharmaceutical composition may be administered to animals, such as mammals (e.g., humans, domesticated animals, cats, dogs, monkeys, mice, rats, etc.). The payload of the pharmaceutical composition is a polynucelotide.

In some embodiments, pharmaceutical, prophylactic, diagnostic, or imaging compositions thereof are administered to humans.

In some embodiments, the herein disclosed pharmaceutical compositions (e.g., LNPs comprising a TnpB gene editing system) are administered by one or more of a variety of routes, including, but not limited to, local, oral, intravenous, intramuscular, intra-arterial, intramedullary, intrathecal, subcutaneous, intraventricular, transdermal, interdermal, rectal, intravaginal, intraperitoneal, topical (e.g., by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter.

In some embodiments, the herein disclosed pharmaceutical compositions (e.g., LNPs comprising a TnpB gene editing system) are administered by systemic intravenous injection.

In some embodiments, the herein disclosed pharmaceutical compositions (e.g., LNPs comprising a TnpB gene editing system) are administered intravenously and/or orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

In specific embodiments, the herein disclosed pharmaceutical compositions (e.g., LNPs comprising a TnpB gene editing system) may be administered in a way which allows the genome editing system to cross the blood-brain barrier, vascular barrier, or other epithelial barrier.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Dosage forms for local, topical and/or transdermal administration of a pharmaceutical composition may include ointments, pastes, creams, lotions, gels, powders, solutions, sprays, inhalants and/or patches. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

In general, the most appropriate route of administration will depend upon a variety of factors including the nature of the genome editing system to be delivered (e.g., its stability in the environment of the gastrointestinal tract, bloodstream, etc), the condition of the patient (e.g., whether the patient is able to tolerate particular routes of administration), etc. The present disclosure encompasses the delivery of the genome editing system by any appropriate route taking into consideration likely advances in the sciences of drug delivery. In certain embodiments, pharmaceutical compositions in accordance with the present disclosure may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administration is employed, split dosing regimens such as those described herein may be used.

According to the present disclosure, administration of the genome editing system in split-dose regimens may produce higher levels of proteins in mammalian subjects. As used herein, a “split dose” is the division of single unit dose or total daily dose into two or more doses. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the genome editing system of the present disclosure are administered to a subject in split doses. In some embodiments, the genome editing system is formulated in buffer only or in a formulation described herein.

The herein disclosed pharmaceutical compositions (e.g., LNPs comprising a TnpB gene editing system) of the present disclosure may be used or administered in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Pharmaceutical compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single pharmaceutical composition or administered separately in different pharmaceutical compositions. In general, it is expected that agents utilized in combination with be utilized at levels that do not exceed the levels at which they are utilized individually. In some embodiments, the levels utilized in combination will be lower than those utilized individually. In one embodiment, the combinations, each or together may be administered according to the split dosing regimens described herein.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a pharmaceutical composition useful for treating cancer in accordance with the present disclosure may be administered concurrently with a chemotherapeutic agent), or they may achieve different effects (e.g., control of any adverse effects).

Pharmaceutical compositions containing LNPs disclosed herein are formulated for administration intramuscularly, transarterially, intraocularly, vaginally, rectally, intraperitoneally, intravenously, intranasally, subcutaneously, endoscopically, transdermally, intramuscularly, intraventricularly, intradermally, intrathecally, topically (e.g. by powders, ointments, creams, gels, lotions, and/or drops), mucosally, nasal, enterally, intratumorally, by intratracheal instillation, bronchial instillation, and/or inhalation; nasal spray and/or aerosol, and/or through a portal vein catheter.

The pharmaceutical compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the pharmaceutical compositions, and the like. In some embodiments, the pharmaceutical composition is formulated for extended release. In specific embodiments, the genome editing systems of the present disclosure and/or pharmaceutical, prophylactic, diagnostic, or imaging compositions thereof, may be administered in a way which allows the genome editing system to cross the blood-brain barrier, vascular barrier, or other epithelial barrier.

In some aspects of the present disclosure, the genome editing system of the present disclosure are spatially retained within or proximal to a target tissue. Provided are methods of providing a pharmaceutical composition to a target tissue of a mammalian subject by contacting the target tissue (which contains one or more target cells) with the pharmaceutical composition under conditions such that the pharmaceutical composition, in particular the genome editing system component(s) of the pharmaceutical composition, is substantially retained in the target tissue, meaning that at least 10, 20, 30, 40, 50, 60, 70, 80, 85, 90, 95, 96, 97, 98, 99, 99.9, 99.99 or greater than 99.99% of the pharmaceutical composition is retained in the target tissue. Advantageously, retention is determined by measuring the amount of a component of the genome editing system present in the pharmaceutical composition that enters one or more target cells. For example, at least 1, 5, 10, 20, 30, 40, 50, 60, 70, 80, 85, 90, 95, 96, 97, 98, 99, 99.9, 99.99 or greater than 99.99% of the genome editing system administered to the subject are present intracellularly at a period of time following administration.

Aspects of the present disclosure are directed to methods of providing a pharmaceutical composition to a target tissue or organ of a mammalian subject, by contacting the target tissue (containing one or more target cells) or organ (containing one or more target cells) with the pharmaceutical composition under conditions such that the pharmaceutical composition is substantially retained in the target tissue or organ. The pharmaceutical composition contains an effective amount of a genome editing system of the present disclosure.

Pharmaceutical compositions which may be administered intramuscularly and/or subcutaneously may include, but are not limited to, polymers, copolymers, and gels. The polymers, copolymers and/or gels may further be adjusted to modify release kinetics by adjusting factors such as, but not limited to, molecular weight, particle size, payload and/or ratio of the monomers. As a nonlimiting example, formulations administered intramuscularly and/or subcutaneously may include a copolymer such as poly (lactic-co-glycolic acid).

Localized delivery of the pharmaceutical compositions described herein may be administered by methods such as, but not limited to, topical delivery, ocular delivery, transdermal delivery, and the like. The pharmaceutical composition may also be administered locally to a part of the body not normally available for localized delivery such as, but not limited to, when a subject's body is open to the environment during treatment. The pharmaceutical composition may further be delivered by bathing, soaking and/or surrounding the body part with the pharmaceutical composition.

However, the present disclosure encompasses the delivery of a genome editing system disclosed herein, and/or pharmaceutical, prophylactic, diagnostic, or imaging compositions thereof, by any appropriate route taking into consideration likely advances in the sciences of drug delivery.

In some embodiments, an LNP composition includes an ionizable lipid, a phospholipid, a PEG lipid, and a structural lipid. In certain embodiments, the lipid component of the nanoparticle composition includes about 30 mol % to about 60 mol % ionizable lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the nanoparticle composition includes about 35 mol % to about 55 mol % ionizable lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % ionizable lipid, about 10 mol % phospholipid, about 38.5 mol % structural lipid, and about 1.5 mol % of PEG lipid. In another particular embodiment, the lipid component includes about 40 mol % ionizable lipid, about 20 mol % phospholipid, about 38.5 mol % structural lipid, and about 1.5 mol % of PEG lipid. In another particular embodiment, the lipid component includes about 48.5 mol % ionizable lipid, about 10 mol % phospholipid, about 40 mol % structural lipid, and about 1.5 mol % of PEG lipid. In another particular embodiment, the lipid component includes about 48.5 mol % ionizable lipid, about 10 mol % phospholipid, about 39 mol % structural lipid, and about 2.5 mol % of PEG lipid. In some embodiments, the phospholipid may be DOPE or DSPC. In other embodiments, the PEG lipid may be PEG-DMG and/or the structural lipid may be cholesterol. The amount of a genome editing system payload in a nanoparticle composition may depend on the size, composition, desired target and/or application, or other properties of the nanoparticle composition as well as on the properties of the genome editing system. For example, the amount of genome editing system useful in a nanoparticle composition may depend on the size, sequence, and other characteristics of the genome editing system. The relative amounts of genome editing system and other elements (e.g., lipids) in a nanoparticle composition may also vary. In some embodiments, the wt/wt ratio of the lipid component to an enzyme in a nanoparticle composition is about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. The amount of a enzyme in a nanoparticle composition may, for example, be measured using absorption spectroscopy (e.g., ultraviolet-visible spectroscopy).

In some embodiments, an LNP composition containing a genome editing system of the present disclosure, comprising a genome editing system is formulated to provide a specific E: P ratio. The E: P ratio of the composition refers to the molar ratio of nitrogen atoms in one or more lipids to the number of phosphate groups in an RNA. In general, a lower E: P ratio is preferred. The one or more enzymes, lipids, and amounts thereof may be selected to provide an E: P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the E: P ratio is about 2:1 to about 8:1. In other embodiments, the E: P ratio is from about 5:1 to about 8:1. For example, the E: P ratio may be about 5.0:1, about 5.5:1, about 5.67:1, about 6.0:1, about 6.5:1, or about 7.0:1.

The characteristics of an LNP (or “nanoparticles”) composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition. Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure Zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, Such as particle size, polydispersity index, and Zeta potential.

The mean size of an LNP composition may be between 10s of nm and 100s of nm, e.g., measured by dynamic light scattering (DLS). For example, the mean size is about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition is about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition is about 70 nm to about 100 nm. In a particular embodiment, the mean size may be about 80 nm. In other embodiments, the mean size may be about 100 nm.

A LNP composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25.

The Zeta potential of a LNP composition may be used to indicate the electrokinetic potential of the composition. For example, the Zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the Zeta potential of a nanoparticle composition is about-10 mV to about +20 mV, from about-10 mV to about +15 mV, from about-10 mV to about +10 mV, from about-10 mV to about +5 mV, from about-10 mV to about 0 mV, from about-10 mV to about-5 mV, from about-5 mV to about +20 mV, from about-5 mV to about +15 mV, from about-5 mV to about +10 mV, from about-5 mV to about +5 mV, from about-5 mV to about 0 mV, from about 0 mV, to about +20 mV, from about 0 mV to about +15 mV, from about 0 mV to about +10 mV, from about 0 mV to about +5 mV, from about +5 mV to about +20 mV, from about +5 mV, to about +15 mV, or from about +5 mV to about +10 mV.

The efficiency of encapsulation of an LNP payload describes the amount of payload that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of payload in a solution containing the nanoparticle composition before and after breaking up the nanoparticle composition with one or more organic solvents or detergents. Fluorescence may be used to measure the amount of free payload in a solution. For the nanoparticle compositions described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

Lipids and their method of preparation are disclosed in, e.g., U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/117528, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548 and Semple et. al, Nature Biotechnology, 2010, 28, 172-176, the full disclosures of which are herein incorporated by reference in their entirety for all purposes.

An LNP-based nucleobase editing systems, RNA therapeutics and pharmaceutical compositions may include any substance useful in pharmaceutical compositions. For example, the nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21 Edition, A. R. Gennaro: Lippincott, Williams & Wilkins, Baltimore, Md., 2006).

The LNP-based nucleobase editing systems, RNA therapeutics and pharmaceutical compositions thereof described herein may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

In some embodiments, compositions may be administered in a way which allows them to cross the blood-brain barrier, vascular barrier, or other epithelial barrier. The originator constructs, benchmark constructs, and targeting systems may be administered in any suitable form, either as a liquid solution or suspension, as a solid form suitable for liquid solution or suspension in a liquid solution. The originator constructs, benchmark constructs, and targeting systems may be formulated with any appropriate and pharmaceutically acceptable excipient.

In some embodiments, the originator constructs, benchmark constructs, and targeting systems may be delivered to a subject via a single route administration.

In some embodiments, the originator constructs, benchmark constructs, and targeting systems may be delivered to a subject via a multi-site route of administration. A subject may be administered at 2, 3, 4, 5, or more than 5 sites.

In some embodiments, a subject may be administered the originator constructs, benchmark constructs, and targeting systems using a bolus infusion.

In some embodiments, a subject may be administered originator constructs, benchmark constructs, and targeting systems using sustained delivery over a period of minutes, hours, or days. The infusion rate may be changed depending on the subject, distribution, formulation or another delivery parameter.

In some embodiments, the originator constructs, benchmark constructs, and targeting systems may be delivered by intramuscular delivery route. Non-limiting examples of intramuscular administration include an intravenous injection or a subcutaneous injection.

In some embodiments, the originator constructs, benchmark constructs, and targeting systems may be delivered by oral administration. Non-limiting examples of oral delivery include a digestive tract administration and a buccal administration.

In some embodiments, the originator constructs, benchmark constructs, and targeting systems may be delivered by intraocular delivery route. A non-limiting example of intraocular delivery include an intravitreal injection.

In some embodiments, the originator constructs, benchmark constructs, and targeting systems may be delivered by intranasal delivery route. Non-limiting examples of intranasal delivery include nasal drops or nasal sprays.

In some embodiments, the originator constructs, benchmark constructs, and targeting systems may be administered to a subject by peripheral injections. Non-limiting examples of peripheral injections include intraperitoneal, intramuscular, intravenous, conjunctival, or joint injection.

In some embodiments, the originator constructs, benchmark constructs, and targeting systems may be delivered by injection into the cerebrospinal fluid. Non-limiting examples of delivery to the cerebrospinal fluid include intrathecal and intracerebroventricular administration.

In some embodiments, the originator constructs, benchmark constructs, and targeting systems may be delivered by systemic delivery. As a non-limiting example, the systemic delivery may be by intravascular administration.

In some embodiments, the originator constructs, benchmark constructs, and targeting systems may be administered to a subject by intracranial delivery.

In some embodiments, the originator constructs, benchmark constructs, and targeting systems may be administered to a subject by intraparenchymal administration.

In some embodiments, the originator constructs, benchmark constructs, and targeting systems may be administered to a subject by intramuscular administration.

In some embodiments, the originator constructs, benchmark constructs, and targeting systems are administered to a subject and transduce muscle of a subject. As a non-limiting example, the originator constructs, benchmark constructs, and targeting systems are administered by intramuscular administration.

In some embodiments, the originator constructs, benchmark constructs, and targeting systems may be administered to a subject by intravenous administration.

In some embodiments, the originator constructs, benchmark constructs, and targeting systems may be administered to a subject by subcutaneous administration.

In some embodiments, the originator constructs, benchmark constructs, and targeting systems may be administered to a subject by topical administration.

In some embodiments, the originator constructs, benchmark constructs, and targeting systems may be delivered by more than one route of administration.

The originator constructs, benchmark constructs, and targeting systems described herein may be co-administered in conjunction with one or more originator constructs, benchmark constructs, targeting systems, or therapeutic agents or moieties.

In some embodiments, pharmaceutical compositions and/or formulations described herein may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly (anhydrides). Depot injectable formulations are prepared by entrapping the drug in liposomes or microemulsions which are compatible with body tissues.

In some embodiments, pharmaceutical compositions and/or formulations described herein may be formulated for administration topically. The skin may be an ideal target site for delivery as it is readily accessible. Three routes are commonly considered to deliver pharmaceutical compositions and/or formulations described herein to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions).

In some embodiments, pharmaceutical compositions and/or formulations described herein may be delivered using a variety of dressings (e.g., wound dressings) or bandages (e.g., adhesive bandages) for conveniently and/or effectively carrying out methods described herein. Typically dressing or bandages may comprise sufficient amounts of pharmaceutical compositions and/or formulations described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, contemplated herein is the use of transdermal patches, which often have the added advantage of providing controlled delivery of pharmaceutical compositions and/or formulations described herein to the body. Such dosage forms may be prepared, for example, by dissolving and/or dispensing pharmaceutical compositions and/or formulations described herein in the proper medium. Alternatively, or additionally, rates may be controlled by either providing rate controlling membranes and/or by dispersing pharmaceutical compositions and/or formulations described herein in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

In some embodiments, pharmaceutical compositions and/or formulations described herein may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations which are useful include those which comprise active ingredients in microcrystalline form and/or in liposomal preparations. Subretinal inserts may also be used as forms of administration.

In some embodiments, pharmaceutical compositions and/or formulations described herein may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

In some embodiments, pharmaceutical compositions and/or formulations described herein are formulated in depots for extended release.

In some embodiments, pharmaceutical compositions and/or formulations described herein are spatially retained within or proximal to target tissues. Provided are methods of providing pharmaceutical compositions and/or formulations described herein to target tissues of mammalian subjects by contacting target tissues (which comprise one or more target cells) with pharmaceutical compositions and/or formulations described herein under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions and/or formulations described herein that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of pharmaceutical compositions and/or formulations described herein administered to subjects are present intracellularly at a period of time following administration. For example, intramuscular injection to mammalian subjects may be performed using aqueous compositions comprising an active ingredient and one or more transfection reagents, and retention is determined by measuring the amount of active ingredient present in muscle cells.

In some embodiments, provided are methods for delivering pharmaceutical compositions and/or formulations described herein to target tissues of mammalian subjects, by contacting target tissues (comprising one or more target cells) with pharmaceutical compositions and/or formulations described herein under conditions such that they are substantially retained in such target tissues. Pharmaceutical compositions and/or formulations described herein comprise enough active ingredient such that the effect of interest is produced in at least one target cell. In some embodiments, pharmaceutical compositions and/or formulations described herein generally comprise one or more cell penetration agents, although “naked” formulations (such as without cell penetration agents or other agents) are also contemplated, with or without pharmaceutically acceptable carriers.

In some embodiments, pharmaceutical compositions and/or formulations described herein may be prepared, packaged, and/or sold in formulations suitable for pulmonary administration. In some embodiments, such administration is via the buccal cavity. In some embodiments, formulations may comprise dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self-propelling solvent/powder dispensing containers may be used. In such embodiments, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, propellants may constitute 50% to 99.9% (w/w) of the composition, and active ingredient may constitute 0.1% to 20% (w/w) of the composition. Propellants may further comprise additional ingredients such as liquid non-ionic and/or solid anionic surfactant and/or solid diluent (which may have particle sizes of the same order as particles comprising active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

In some embodiments, pharmaceutical compositions and/or formulations described herein may be administered nasally and/or intranasal. In some embodiments, formulations described herein useful for pulmonary delivery may also be useful for intranasal delivery. In some embodiments, formulations for intranasal administration comprise a coarse powder comprising the active ingredient and having an average particle from about 0.2 μm to 500 μm. Such formulations are administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

In some embodiments, pharmaceutical compositions and/or formulations described herein may be administered rectally and/or vaginally. Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

In some embodiments, a nanoparticle includes an ionizable lipid, a phospholipid, a PEG lipid, and a structural lipid. In certain embodiments, the lipid component of the nanoparticle composition includes about 30 mol % to about 60 mol % ionizable lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the nanoparticle composition includes about 35 mol % to about 55 mol % ionizable lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % ionizable lipid, about 10 mol % phospholipid, about 38.5 mol % structural lipid, and about 1.5 mol % of PEG lipid. In another particular embodiment, the lipid component includes about 40 mol % ionizable lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol. The amount of editing system components in a nanoparticle composition may depend on the size, composition, desired target and/or application, or other properties of the nanoparticle composition as well as on the properties of the editing system. For example, the amount of editing system useful in a nanoparticle composition may depend on the size, sequence, and other characteristics of the editing system. The relative amounts of editing enzymes, or polynucleotides encoding said enzymes and other elements (e.g., lipids) in a nanoparticle composition may also vary. In some embodiments, the wt/wt ratio of the lipid component to a polynucleotide in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. The amount of a polynucleotide in a nanoparticle composition may, for example, be measured using absorption spectroscopy (e.g., ultraviolet-visible spectroscopy).

In some embodiments, a nanoparticle composition containing an editing system of the present disclosure, comprising a polynucleotide may be formulated to provide a specific N: P ratio. The N: P ratio of the composition refers to the molar ratio of nitrogen atoms in one or more lipids to the number of phosphate groups in a polynucleotide. In general, a lower N: P ratio is preferred. The one or more polynucleotides, lipids, and amounts thereof may be selected to provide an N: P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N: P ratio may be from about 2:1 to about 8:1. In other embodiments, the N: P ratio is from about 5:1 to about 8:1. For example, the N: P ratio may be about 5.0:1, about 5.5:1, about 5.67:1, about 6.0:1, about 6.5:1, or about 7.0:1.

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition. Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure Zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, Such as particle size, polydispersity index, and Zeta potential.

The mean size of a nanoparticle composition may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition may be from about 70 nm to about 100 nm. In a particular embodiment, the mean size may be about 80 nm. In other embodiments, the mean size may be about 100 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25.

The Zeta potential of a nanoparticle composition may be used to indicate the electrokinetic potential of the composition. For example, the Zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the Zeta potential of a nanoparticle composition may be from about-10 mV to about +20 mV, from about-10 mV to about +15 mV, from about-10 mV to about +10 mV, from about −10 mV to about +5 mV, from about-10 mV to about 0 mV, from about-10 mV to about-5 mV, from about-5 mV to about +20 mV, from about-5 mV to about +15 mV, from about-5 m V to about +10 mV, from about-5 mV to about +5 mV, from about-5 mV to about 0 mV, from about 0 mV, to about +20 mV, from about 0 mV to about +15 mV, from about 0 mV to about +10 mV, from about 0 mV to about +5 mV, from about +5 mV to about +20 mV, from about +5 mV, to about +15 mV, or from about +5 mV to about +10 mV.

The efficiency of encapsulation of a payload describes the amount of payload that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of payload in a solution containing the nanoparticle composition before and after breaking up the nanoparticle composition with one or more organic solvents or detergents. Fluorescence may be used to measure the amount of free payload in a solution. For the nanoparticle compositions described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

Lipids and their method of preparation are disclosed in, e.g., U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/117528, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548 and Semple et. al, Nature Biotechnology, 2010, 28, 172-176, the full disclosures of which are herein incorporated by reference in their entirety for all purposes.

A nanoparticle composition may include any substance useful in pharmaceutical compositions. For example, the nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21 Edition, A. R. Gennaro: Lippincott, Williams & Wilkins, Baltimore, Md., 2006).

The pharmaceutical composition may be delivered as described in PCT Publication WO2012135805, which is incorporated herein by reference in its entirety.

The present disclosure provides methods comprising administering a pharmaceutical composition to a subject in need thereof. The pharmaceutical composition may be administered to a subject using any amount and any route of administration which may be effective for preventing, treating, diagnosing, or imaging a disease, disorder, and/or condition. The exact amount required will vary from subject to subject, depending on factors such as, but not limited to, the species, age, and general condition of the subject, the severity of the disease, the particular composition, its mode of administration, its mode of activity, and the like. The pharmaceutical composition may be administered to animals, such as mammals (e.g., humans, domesticated animals, cats, dogs, monkeys, mice, rats, etc.). The payload of the pharmaceutical composition is a polynucelotide.

In some embodiments, pharmaceutical, prophylactic, diagnostic, or imaging compositions thereof are administered to humans.

In some embodiments, the editing system is administered by one or more of a variety of routes, including, but not limited to, local, oral, intravenous, intramuscular, intra-arterial, intramedullary, intrathecal, subcutaneous, intraventricular, transdermal, interdermal, rectal, intravaginal, intraperitoneal, topical (e.g., by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter.

In some embodiments, editing systems are administered by systemic intravenous injection.

In some embodiments, editing systems are administered intravenously and/or orally.

In specific embodiments, editing systems may be administered in a way which allows the editing systems to cross the blood-brain barrier, vascular barrier, or other epithelial barrier.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Dosage forms for local, topical and/or transdermal administration of a pharmaceutical composition may include ointments, pastes, creams, lotions, gels, powders, solutions, sprays, inhalants and/or patches. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

In general, the most appropriate route of administration will depend upon a variety of factors including the nature of the editing systems to be delivered (e.g., its stability in the environment of the gastrointestinal tract, bloodstream, etc), the condition of the patient (e.g., whether the patient is able to tolerate particular routes of administration), etc. The present disclosure encompasses the delivery of the editing systems by any appropriate route taking into consideration likely advances in the sciences of drug delivery.

In certain embodiments, pharmaceutical compositions in accordance with the present disclosure may be administered at dosage levels sufficient to deliver from about 0.0001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administration is employed, split dosing regimens such as those described herein may be used.

According to the present disclosure, administration of editing systems in split-dose regimens may produce higher levels of proteins in mammalian subjects. As used herein, a “split dose” is the division of single unit dose or total daily dose into two or more doses. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the editing systems of the present disclosure are administered to a subject in split doses. The editing systems may be formulated in buffer only or in a formulation described herein.

Editing systems may be used or administered in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Pharmaceutical compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single pharmaceutical composition or administered separately in different pharmaceutical compositions. In general, it is expected that agents utilized in combination with be utilized at levels that do not exceed the levels at which they are utilized individually. In some embodiments, the levels utilized in combination will be lower than those utilized individually. In one embodiment, the combinations, each or together may be administered according to the split dosing regimens described herein.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a pharmaceutical composition useful for treating cancer in accordance with the present disclosure may be administered concurrently with a chemotherapeutic agent), or they may achieve different effects (e.g., control of any adverse effects).

Pharmaceutical compositions containing editing systems are formulated for administration intramuscularly, transarterially, intraocularly, vaginally, rectally, intraperitoneally, intravenously, intranasally, subcutaneously, endoscopically, transdermally, intramuscularly, intraventricularly, intradermally, intrathecally, topically (e.g. by powders, ointments, creams, gels, lotions, and/or drops), mucosally, nasal, enterally, intratumorally, by intratracheal instillation, bronchial instillation, and/or inhalation; nasal spray and/or aerosol, and/or through a portal vein catheter.

The pharmaceutical compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the pharmaceutical compositions, and the like. In some embodiments, the pharmaceutical composition is formulated for extended release. In specific embodiments, editing systems and/or pharmaceutical, prophylactic, diagnostic, or imaging compositions thereof, may be administered in a way which allows the editing systems to cross the blood-brain barrier, vascular barrier, or other epithelial barrier.

In some aspects of the present disclosure, the editing systems of the present disclosure are spatially retained within or proximal to a target tissue. Provided are methods of providing a pharmaceutical composition to a target tissue of a mammalian subject by contacting the target tissue (which contains one or more target cells) with the pharmaceutical composition under conditions such that the pharmaceutical composition, in particular the editing system component(s) of the pharmaceutical composition, is substantially retained in the target tissue, meaning that at least 10, 20, 30, 40, 50, 60, 70, 80, 85, 90, 95, 96, 97, 98, 99, 99.9, 99.99 or greater than 99.99% of the pharmaceutical composition is retained in the target tissue. Advantageously, retention is determined by measuring the amount of a component of the editing system present in the pharmaceutical composition that enters one or more target cells. For example, at least 1, 5, 10, 20, 30, 40, 50, 60, 70, 80, 85, 90, 95, 96, 97, 98, 99, 99.9, 99.99 or greater than 99.99% of the editing system components administered to the subject are present intracellularly at a period of time following administration.

Aspects of the present disclosure are directed to methods of providing a pharmaceutical composition to a target tissue or organ of a mammalian subject, by contacting the target tissue (containing one or more target cells) or organ (containing one or more target cells) with the pharmaceutical composition under conditions such that the pharmaceutical composition is substantially retained in the target tissue or organ. The pharmaceutical composition contains an effective amount of an editing system.

Pharmaceutical compositions which may be administered intramuscularly and/or subcutaneously may include, but are not limited to, polymers, copolymers, and gels. The polymers, copolymers and/or gels may further be adjusted to modify release kinetics by adjusting factors such as, but not limited to, molecular weight, particle size, payload and/or ratio of the monomers. As a nonlimiting example, formulations administered intramuscularly and/or subcutaneously may include a copolymer such as poly(lactic-co-glycolic acid).

Localized delivery of the pharmaceutical compositions described herein may be administered by methods such as, but not limited to, topical delivery, ocular delivery, transdermal delivery, and the like. The pharmaceutical composition may also be administered locally to a part of the body not normally available for localized delivery such as, but not limited to, when a subject's body is open to the environment during treatment. The pharmaceutical composition may further be delivered by bathing, soaking and/or surrounding the body part with the pharmaceutical composition.

However, the present disclosure encompasses the delivery of an editing system disclosed herein, and/or pharmaceutical, prophylactic, diagnostic, or imaging compositions thereof, by any appropriate route taking into consideration likely advances in the sciences of drug delivery.

Provided herein are methods of treating a disease or disorder, the methods comprising administering to a subject in need thereof a pharmaceutical composition of the present disclosure. In various embodiments of the invention, target genome modifications include cells with monogenic diseases or disorders. Various monegenic diseases include but are not limited to: Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylkeotnuria; Severe Combined Immunodeficiency; Sickle Cell Disease; Smith-Lemli-Opitz Syndrome; and Tay-Sachs Disease.

Accordingly, the invention provides production of various TnpB molecules suitable to for gene therapy to ameliorate or cure such monogenic diseases or disorders.

### G. Sequences

The following sequences are part of the specification:

| SEQ ID NOs: | Description | Location |
|---|---|---|
| 1-135 | TnpB amino acid sequences | Table A |
| 136-190 | Linker sequences | Table D |
| 200-208 | Accessory amino acid sequences | Table C |
| 209-239 | TnpB coding + reRNA − | Table B |
| | nucleotide sequence | |
| 240-270 | TnpB coding − nucleotide | Table B |
| | sequence | |
| 271-301 | reRNA − nucleotide sequence | Table B |
| 302-381; | Various | Throughout |
| 398-449 | | specification |
| 382-397 | Example 11 sequences | Example 11 |

TABLE A
TnpB protein sequences

| SEQ | | |
|---|---|---|
| ID | Description | Amino Acid Sequence |
| 1 | Deinococcus | MIRNKAFVVRLYPNAAQTELINRTLGSARFVYNHFLARRIAAYKESGKG |
| | radiodurans | LTYGQTSSELTLLKQAEETSWLSEVDKFALQNSLKNLETAYKNFFRTVK |
| | TnpB full | QSGKKVGFPRFRKKRTGESYRTQFTNNNIQIGEGRLKLPKLGWVKTKGQ |
| | sequence | QDIQGKILNVTVRRIHEGHYEASVLCEVEIPYLPAAPKFAAGVDVGIKDF |
| | (ISDra2) | AIVTDGVRFKHEQNPKYYRSTLKRLRKAQQTLSRRKKGSARYGKAKTK |
| | | LARIHKRIVNKRQDFLHKLTTSLVREYEIIGTEHLKPDNMRKNRRLALSIS |
| | | DAGWGEFIRQLEYKAAWYGRLVSKVSPYFPSSQLCHDCGFKNPEVKNL |
| | | AVRTWTCPNCGETHDRDENAALNIRREALVAAGISDTLNAHGGYVRPAS |
| | | AGNGLRSENHATLVV* |
| 2 | Staphylococcus | MNASSKRKIISQSEISKKIAVMNEEMQGFWANNSWDIRKCPHPSAIELSK |
| | aureus TnpB full | NPALRNRWVRFERVKNLWLRTELKYFYFYHLNNGIWNAKTVWIRKGTV |
| | sequence | INKMLDFLDLKYPSITSITEVPIDKAMTEYRTYLTKQGVRITTTNYKITAN |
| | | QEKTPVKANSYYVTNLKQFIEFIEDFYFDGEEWDKDVWDRRNLPLPDDK |
| | | VNPTQYEYTINFKGFRNTYFKQLVKRYCKLRLNMDSFSYVSDIAQKLKE |
| | | FFNFLDIKFKHVQRVHQLTRVEIEAYLSELNMMGIKPSTITGRISILEGLFS |
| | | TLHRLEWDDVPSKLLIYPEDYPKIPRAKPRFIDEFVLEQLNSHLDKLPEY |
| | | IATMTMIVQECGMRISELCTLKKGCLLEDKDGYYFLKYYQWKMKK |
| | | EHIVPISKEVVLLIKVREDKVSEEFPDSEYLFPRKDGSPLKQETFRGEL |
| | | NKLAYEQNIVDKLGEIYRFHAHAFRHSVGTRTINNGVPQHIVQKFLG |
| | | HESPEMTSRYAHIFDETLKNEFTKFQEKLVTNNGDVLNLDDDSEVDDV |
| | | ELQWFKKNINAQVLPNGYCRLPVIAGGCPHANACLDCTHFCTSKQFLPQ |
| | | HEEQLERTEELLTIAKDKQWQRQIETNSRVKERLEQIIGSLTG |
| 3 | Staphylococcus | TYFKQLVKRYCKLRLNMDSFSYVSDIAQKLKEFFNFLDIKFKHVQRVHQ |
| | aureus TnpB | LTRVEIEAYLSELNMMGIKPSTITGRISILEGLFSTLH |
| | Core binding | |
| | domain | |
| 4 | Staphylococcus | AKPRFIDEFVLEQLNSHLDKLPEYIATMTMIVQECGMRISELCTLKK |
| | aureus TnpB Tyr | GCLLEDKDGYYFLKYYQWKMKKEHIVPISKEVVLLIKVREDKVSEE |
| | recombinase | FPDSEYLFPRKDGSPLKQETFRGELNKLAYEQNIVDKLGEIYRFHAH |
| | domain | AFRHSVGTRTINNGVPQHIVQKFLGHESPEMTSRYAHIFDETLKNEFT |
| 5 | MDe-1 TnpB/ | MPSTSSCKEPKSTSKPSTTSNAKKQNTKTNDKPQNDKIKKARVVKDNTF |
| | Fanzor | HHYTLVRGLNNMIRPEKSEQLMFAINELVPLVSKSMVLGSWHTHVRLME |
| | | IELEANRIEQETGIRVENQLGIRAEFTPPSVINARGQVRKIGYVVEKDFNPT |
| | | KQTRGINLIHTEPINANFKRHLDVYDVDIPHLPYGANCHNYATNQYTTNF |
| | | HNNIRYRAYNRVRAFFNVHSNDRKSKRETLKYLFYVEPKKRKAIPDPLPI |
| | | PNDELLKLYRKVLRPLLPNDAPDRVPLGWLGASLRDKIDWYRHVHEFIQ |
| | | LQLWIYSDNLRRDGTDEIQLRSFTAIPQNKVKRHHITITTRILGGIVNSFEC |
| | | RAEKISSKDYESSDVAIRTRIWNKYFRLEWLNSEWRYFDLTMTTDGVSA |
| | | HFVMRNKQKQKSKREVVDVNGDVLTPEIVGFIDDRIVREISDEMRQKYN |
| | | NNEYKTIIGYDPGYKLPVAIVRKDIATGDETNFTMNWYEFRKLCGDVER |
| | | KQKRYRVAGKHEKKMRQLRENRTIYPEQPSNKSHDIWTYIDYQCRVFHR |
| | | SCAVYMRRSYARLEFDRYICTQRALHKIARIIMPRVDFKNPTRDCILAAG |
| | | DFKFSANSPIRGSVRCPMRSLFNLLAQQYCDVVEIDEHLTSKLCSICGSRM |
| | | IKKGTRKDRYFICKNKMPATSILPQAPAWTTKRHIDRIRDEFNELPLAGLF |
| | | IHQDNGGRRIKRTIDLPTERRFHSNELDDHHYQLQQHEIINGNIVAKHKTI |
| | | VWNRDINAPRNMIYKLLCMLNGVDISPVFKRRPRLAEQQQQQQQQAND |
| | | NIDDTDSGSSSSDEDMR |
| 6 | MDe-2 TnpB/ | MAQKAVVCAYKKKFRQVNEFNINPNQIIAEHRRHAEQLTRLKRKASEHI |
| | Fanzor | FFRLTSLVREGTGHQILLEFQTPNRVNFRRDINSVRITERNARSEPNAEFVE |
| | | GSRVYQIHPEGNIVAQSIEPIITEFEVNFANSFIGECYGRVKRLLKCFARIHL |
| | | ENMEDDQIKIEIRNTMRYLFNDINDITEPNRTLIRLYRQELRPICRGQVPRG |
| | | WWNNKSTWYSRVPDLINGLYYLKAYNDGQRIGRDCFKLFTVIPRFKSHF |
| | | VHKTKIDTRLLCLTMNKFLPEGQKLDSQNIDYTVWNRVFDMTPLERTHT |
| | | DRKLVFNYSITTDAVSMSATMIQGTDREIEAKRELRGRGAKLAERKRLA |
| | | DERKMEESTKATKRKAGKDADEPPAKRRNAPVPTRERDLLQKLKSGEVF |
| | | GTVIQIDPGVKNTLGCVAIDMQNGHERNLLITSKYYHHLLQTHLRKERK |
| | | RPIIAHLHAMIRGVHASYANPPTRKGGDIREYLDSVFRLFDMFTEVYQRR |
| | | VFSRMRFDAHICKQKAVDKISKQIASLNPHQESATLIMFGNSQMSRKSPIK |
| | | GYVKSPHKNYWVKQFDELERVEVYEVDEYRTTKTCSFCGQILHVTRNIG |
| | | HRYVSCKDCYPAGNASMVNEPKEVQTKSKQYWRRHHYRVNGIRTFSYS |
| | | WEERMSHRKCRTTNRDINAPRNIGLKGFATITGRENLLPVALIRANDND |
| 7 | MDe-3 TnpB/ | MLRTMYQHSLLWPLITFFLNFANWFDRTIIMIWRKNFIAKQTANMFWDY |
| | Fanzor | FYEIKFWIHDKSSNLDRDYAKMLTKSVKNKPNLKNLNQPIKFVINQFKM |
| | | NFENSTKMHAEKHVKKFIKCVNKQFFRDKNDEQRTEIIENAISFLFDKKS |
| | | DCKINKLVLREINNHVRVCLGQITRGYFATMKSSGWYKFTPENIKMQWY |
| | | INRYNLAYPDDQLPSFKAVPTHNLGLKCVRIDTVSLKDLFPIPVKDYKNP |
| | | KSELWSRYFNVSPIIGDRKNLYFDNSIITNGPYTNFQFIISTTPINTDLPKKII |
| | | GQNASCATGKDFKIATINDSIRQSVRNNEFDVITAIDIGFNNMAGTTSLFS |
| | | FDISELSLNYTSKWFRSIAGFMWHKLERERITQVFQEYEKDLRESHQYQP |
| | | SVNAVDFMLYIRHAYHRNDLARLRFSQQQMKQKAVATLGYSIIGTRPDG |
| | | TKNNALIGIGAMHFSGSSPIKGHGRVPHKALCAYLKDRSTFIRVSEFRSTQ |
| | | ACCFCGKMSVESSGKDRYVVCKDCAPNQNFLLPPAKLFTVKTHNNFRRL |
| | | YGFKLPFRSYERTNSNSMINTRNTNRDLCGSRNILQIMLYSVHKQPLPGH |
| | | LNNSNDPKKVKKVSPKIKK |
| 8 | MDe-4 TnpB/ | MHFSTPYINRFWRFKNHSAAGHHKKQKYIQVNALTIVHCIHFINEIFSKRT |
| | Fanzor | VVFSVFFKWFCCKMRQFTIKTTLRSICYDEDLIAAIDRLSRDVSIIQFLASH |
| | | HLHKRSMEIIQSENPFRIRREFDREVTDLMFKNDFNAVKANTRNPVADAQ |
| | | FLRWMANCNPPVQLPSGLHSGNVLQEAMKMYKTNYSNNIKMRAKSMF |
| | | RKYCKAVLYNTGRVWPQRVLNKLINLAVDWFFFFTNYQPRRGSPVLTIIE |
| | | EFTRNIRINNEAVDRGFLSRSLDGRDFYEFSKYFIKMQLFVYEYNDMHPD |
| | | RKVKSFSVIPLHNYHRRCVLIDTETLRKLTHMHMDKKQPNHLGNLNPKK |
| | | SDVWTHFDMSQVDYKRDNQRFDFKITSDGFQANILMMKLRNDELHVDL |
| | | DTPSKMKGQSMRPKDLPMLENFDQIPAEVRARVDEYHVKIGIDPGAKTM |
| | | ASWTKQYRSLAEDCDSIESKTFREMFGFREREYNRRKITRWFDSRERRAR |
| | | EESDIPITRKGPHYTEFVQHKLEFCNAGFAVYHDPELRKMKFDAHQLRLK |
| | | AMYAVAKRIIGYSGNKKLNEALIFFGHWRWTANSPIRGIRTPLKLLRFCL |
| | | QQIGSTVIVIHEHRTSKLCNFCGQTMIHDDRNSLCIHCEPIERTLAPATVW |
| | | QMRAHKSFLKRHPFAPRMRLYTRDPNQPMRRNNQGHNPMNWNRDFNA |
| | | SRAILSKGLDLVAGRPLPRNYLQTTQRVVQRVRFPRR |
| 9 | MDe-5 TnpB/ | MRRFTIKTTLRSICESDRLIADIKTMAEDISKIQTLASLHIHKRSMDIILSND |
| | Fanzor | PVRIRAEFDQPVEGKMFRKDFHAVKANTRNRVADAEFMEWVRDAGIQL |
| | | PRARNTDQALISSYEMYKTNYSNHIKMRAKQMFRKFCKSVLRRFYQQRE |
| | | RWYNEQLNRAVEYFFYFTNYRPTRFIQKIIQEFNQFIRIENENVGRGFMNH |
| | | SLDGDYFYEFAKYFLKMKLYIHRYNDAHPNRKVQTIKVIPIHNFHRRCITI |
| | | DTSTLRKLTHTYGNSPLAALNPRKSDVWSDFDMSSLDIKDQYFDYQITTD |
| | | GFAANICMIKSRVYVDTELPSKIKGVNMRVFEPPAQEPPFDQIPDEVIERID |
| | | QFDLQIGIDPGGKTMAAVTQKFPNDDENCFSIEAKKYREMTGFRTREYN |
| | | RRGITGYFDEWEKQIRQQSYIPITSKGRHYREFVQHKLDMCDWGWEAYH |
| | | HPKVIQMKFDAHQQKLKAMYAVAKSIIGYSAENQRWNEALIFFGNWTW |
| | | KANSPISGTRTPIRLLRFCLQQLGSTVIVIDEYRTSMLCNFCSLRMIEEGRN |
| | | ELCVDCVPIDRSLAPAAVWQIKAHKSYLKRNPGAYKIRTFTRNPNQPLRR |
| | | NHQNHSPMNWNRDYNASRAILSKGLDLVAGRQVPHNYLRTTPNVPRRQ |
| | | RFRRVR |
| 10 | MDe-6 TnpB/ | MRQFVIKRKLRSILNDADLINRIDQIAENVSNIQFLASHYLHKRSMQVILS |
| | Fanzor | EDAALIRREFDRPVRDLMFKSDFFAVKANTRNAERDADFMQWLADVNP |
| | | PILLPDALHCDNALQHAMKMYKTNYANNIKMRAKQMFRKFCKAVLYST |
| | | GRIWPRIVLNKLINRAVDWFFYFTRYEPNDRSPVRTIIDEFIWDILIDDEDV |
| | | GRGFLSGSLDARNFYQFAKYFIKMQLYVHHYNDTHPERMVKSFKVIPLH |
| | | NYHKRMVMIDTETMKKMRHMHFDKKIHNINPPKSDVWAPFRLEGLEKI |
| | | NQRFDFKIMSDGVTAAILMMKLNPHEFHIDLNLPSKINGVSTRPKDLPLK |
| | | ERFDHIPRGAIENINFDAKIGIDPGAKTMAACTKQHQNRDLTEECFSIESK |
| | | KYRELAGTRKNEYNRRSITGRFDKREREAREASEILITSNGPDYAEFVRH |
| | | KLEFFNEGRRVYHHPYVRELKFDAHQEKLAAMYAIAKEIIGRSANRWNK |
| | | ALISFGHWRWTANSPIRGIRTPLKLLRFCLQQLGSTVIVIDEHRTSKLCNF |
| | | CAQTMINEDRNSLCVDCRPIQRTLAPAAVWRIKAHKSYLKRNPGAPKIRE |
| | | YTRDPNQQLERNNQNHSPMNWNRDYNGSRAILSKGLDLIAGHPLPRNYL |
| | | RTTQRVVQRRRFARR |
| 11 | MDe-7 TnpB/ | MAVGSKENVSVHLSNDPKEEITVVKRTLNSILSDVNPNKERIQRAIDDRV |
| | Fanzor | DRVTRMSYLASTLFLYECYKAVENRNFAYFDTVDNIKACHYKVTNSTDR |
| | | KYVGRLQQCRDIWNLFQTFGIDQIDTSYFGNIMNYSYGEYTTNCQNNITL |
| | | HFKQRIKRFFKVIANTPNNNLPNFTQETIEDTVKYMNDTRATVQPNLELI |
| | | HQIPYWEELHINDDHINAHPGFFRDFINHGENWFKAIPVFIQIQWAINDYQ |
| | | ILCQTNEELPNFKNFVVIPQNTHKLRHIRIDNQAFFNILAELDIVPPSKKNK |
| | | KRKNDGSLPRMDVVEFIKRKEENWYSLLDEDKIRQSERQKGEKPEIRKPK |
| | | KGRPARRKGRGKPIVDRTKAKAFNQQIVTNGVSASVFYINNQRDRAVKP |
| | | DDYLEEAIPQKLQNDQYRRVIGVDPGYKVWTSAVIRYPHKKKEFNYEVP |
| | | SKKFHGQCGKWKREKKQKELTGEFEASRNRDALDRDLYAETPSANSRQ |
| | | HWFNYIVWRLKFYEEGMAYYATKEYARTDLDKYFQTQKTMDKVTNEL |
| | | VGNDPSLICIGSADFKPNAPIKKYIRCPGTKMLQKYLRKKREVDVLRVDE |
| | | YMTSQTCGYCHDFFDWGLHDELDKKKQRFRICQHANCQPPQEDPIGKV |
| | | DKIIAKRNRRILQKENRAYPHIYFPQEHPNQVEPDRVIANGVEFSNQRLNI |
| | | TNRMIAKTNHVNLTIQNGNQKLIWNRDISAARNILYKGLCAALKKPMAP |
| | | NLDRRNQRQHIAQKREYYAQRRRERELLE |
| 12 | MDe-8 TnpB/ | MSQKRTCPSLQQVILGTRTNILCISFSQATYRRGPHKEAILAAINKRVITM |
| | Fanzor | TRIAYHGSTVALFKCKKALEEGNFAFFNDLKNIRDCFLHVSVAKDVHGNI |
| | | EDYTRNLHDDVRNCFTEFNIEPPSTEALGNVEPYAVNQYITNFADNIRMH |
| | | FKKRLKTFFFARTRRNPLLFPDITPQVIHDTVSYMTNALARGVEPNIDLIH |
| | | EIPNWNQFNISDEYVQQNQGFFEKKLLGTGKDERWFRTVIMFLGMQMEI |
| | | DDYQEWCRAHPELELGMLKNFVVVPLHKLKRQHIRIDTEAFYWLLRELK |
| | | LIPETPGKIHKEKAKFMEDRERVKHWYNWFDKEKIRKMEKGGNFFHFQI |
| | | VTNGLGASVLFTRVNQQSSNKPDNYMESIRQKLESGHYKRVIGMDPGYK |
| | | VWISAVIRYLRTGQEFNYDVTSRQFHKFTKKKKREKKAKRLTGDFENRR |
| | | KADAENRELYPAGIPSPNRRENWFDYIRWRFKWCNEGFDLYTDIQYARL |
| | | DLDQHFRTQKVMDRMVNKLVGNDPALVCIGSTNFNPSAPIKKHIRCPGT |
| | | KMILQYLKKKTKVDILLIDEFNTSQVCGHCHSFFDREWQDRHENRGRRF |
| | | KQCTVANCQIRFPNPIGPTNVIVAKKNHRKIQQERRAFPIVAEHPEGQPEQ |
| | | DPKKSTHQFLKTKYNKIRPYPEGPKPKYVCNVQNNDWGLVTQRGNSKIT |
| | | WNRDTSAARNILYKGVCAVLKIPIHPNFVRPPRMNRALTQAEKQARKFK |
| | | RK |
| 13 | MDe-9 TnpB/ | MDEWAFWEHQDVGNGQLRLRNEALHRLLYETEFEYPKQKEDEIFTLVR |
| | Fanzor | GLAGMLQDEFFFPMMDKINDTVPLITKAIKLGSLHLHFCLFDLVKRGTIR |
| | | EIKEEFLGKFNTIKYFGPTRKTSHQCRYEMNKGFEYYMLAHDVHPPELEY |
| | | STQVYAFAVQEYERNFQTNVKTHAYNRIKRLFDYHSGVGKDKYNALKY |
| | | LFDEDSDIEPNDYVLSLLRVYDEVLQPLTQITPEEKRRRENADEPVIPRGW |
| | | LATLSDKKKWYKHVYEFINLLQWFYHHNIEEPDFQKKLFIAIPQSKIQNH |
| | | LITIDTSAIVDMTNSFLEPGKKTNKTTLEGGLDAPGFKDSFWRTYFKIDQL |
| | | NNGRQYFDFSLQTNGVAVHLKMKKRKLWKPIMKFRSDALPPTSSNIWD |
| | | THEFARMTDRMRMRWANKRYRYLIGFDPGYKLPAAIVRQHCATKNEEN |
| | | FTMHWKEFRNRCGDPRRKRKLRKLTGWYEDLARSIRENRELYPEQPSPK |
| | | SPDYDTYIDYQMRSFNIGCAVYQKESISRLRFDKYICTQRTLHRIAKEITK |
| | | NDKVPKNPEDKNCIIGIGDFSFKPNSKIRGSVRCPMRPLYEILKLYADVVE |
| | | IDEFRTSKTCSICGTQMEKTGGRKDRFFVCHNSDPVGSILPRAPEWTSKLH |
| | | HSKITQIMRRLPEHERYYVHEVTERLPEWRIANLLPDQEEFQVISEIRRQQ |
| | | NAVPKEWTHRNWDFLNWNYDLDQVQHPTIVWNRDINAPRNMIHILKSM |
| | | LNGIEKHPIFNRGDNNNPD |
| 14 | MDe-10 TnpB/ | MMVSTPNSTPNPKPKKARQMKDNTFKHYTLVRGLNKMIRPEKFEKLKG |
| | Fanzor | AIDTLVPLISKSVILGSRHAHVRLMELVQNAMHDEERTGIRAEDHAEIRA |
| | | EFTPPAVRNNKGVLQKHGYVVERDFNPTKRTSKQSLIHNDPIHPNFQLYL |
| | | DVYGVEIPYLPFGVRCHEYAMNQFCTNFHNNIRFHAYNRVRKFFNVHSN |
| | | NTELKRKTLKYLFYVKPKKKNARQVAIPVPDERLLKLYHEVLRPLLPPNI |
| | | NTVPLGWLGESLRDRYDWYRHVHEFMRLQLWIHANNLQCDEEKDQLPS |
| | | FTTIPQYNFKRKFLTLDTKALATIANSFERRAEKTPSLDYGSSDIEIRNRIW |
| | | QRYFKLELLNKEMRHFDYTMTTDGVSAHFVMRNKRKQRTKREILDVEG |
| | | NVIAPEVVGFIDDRIKREPDDEIRTKYANNEYQTIIGYDPGYKLPVAIVRM |
| | | DIDTGNETSFTMHWKEFRKLCGDPERQQRRYRIAGKHELKMRKLRENRII |
| | | YPEQPSNKSPDYWTYMDYQMRTFNQCCAVYMRRSMARMNFDKYICTQ |
| | | RALQKIAKIITRNERCILAVGDFKFSANSPIRGSVRCPMRALFNLLAQQYG |
| | | DVIEIDEYLTTQICSICGSRMQKKGGQKDRIFVCKNRKPADLILPRANTWT |
| | | AKLHKDRIRAQFRRLPEAERYNVQLDNNIIEHRERITLPTERNYQITDNPQ |
| | | RAVIEAGDGDNNVVVVAKHQSIVWNRDLNAPRNMIYKLLCMLNGNAIS |
| | | PVFNRPPPRNNQQADNEQPPLDEVHPQADNEDDEI |
| 15 | MDe-11 TnpB/ | MAKRKGKFINTSKPKRKKKVFETKETSIKVGLHKILRRTKNTKKIIKEIQE |
| | Fanzor | RSVKAAKLSVLSSLLILFEVNNAVYEQESAENIEFERTFFNQDISKYIQTRF |
| | | RSMTQKDSLENNLVYDNFSNLLYNCHIDKPETTAWANSFVYNYEQYET |
| | | NWSTHINIHAKKRFIKFFKTLDDEHTIKEMKDTATFMINSKSRVSPNENLL |
| | | NHLITVGIGPDIHIFSGFKRGFFKRRLNKNQWFQTIPAFVRMQRHIDRYN |
| | | MTKEQERMIRNFAVVPICSYKRRHLRYDGQTIYNLSRRLGILQKGRNRK |
| | | NIPVNRFTDNATRRSCWDREFNMGYLASLGDRIQRNRRKKQFQYQFTTD |
| | | RVACSPLYEKSAQVCEPDILNIKINTCIFTLLYISYENGEFKYGVKVDPGM |
| | | KTYLAAVRQDWTDDNHNFKVGSTEFHRRAKQPDRDEKAAEIVGDLEIR |
| | | RRLDRENNYPRMPSANGLEWMSYITHNLKFFNETIHTYTKPIYTHLAFDN |
| | | YSNTQATMDNIVNELTQRQPTMFFIGGAKFSPASPIGKYVRCPGVRKLTM |
| | | AVKKLPNCGMILVPERNTSQVCPLDLKFFPRQTKKDRFETCYNCQRLPTT |
| | | KPATRIITKVSKRELQRQRRNARLAEQEANLPQEMNDPQINVHQQPQFER |
| | | RERRLVSKFTRYVKINQPQPNADHSDNESNDEMDVDMEANDNPLTTVW |
| | | HRDIAASECIGYVGKC |
| 16 | MDe-12 TnpB/ | MRQFVIKKTLRSICPDEELIAKIDQLSKNVSEIQTLASHHLYKRSMEIILSE |
| | Fanzor | DPERIRREFDRPVIDLMFKKDFFAVKANTRNPEADHEFLQWMANCNPPV |
| | | RPRDALHCDNALNYAMKLYKTNYGNNIIMRAKQMFRKFCKALIYNTGR |
| | | VLPQIVLNRFINRVIEWFFYFGNYEPRGFMRSIIDEIIWQCTIDEEDIGRGFF |
| | | NVLVSERPLNKHPFFYRFAKYFIKMQLYVHHHNDTHPNRRAPSFDVIPLH |
| | | NYHRRFAMIDTETLRKMTGLHMNKKQPNDIGNFNPRKSDVWSCFYSED |
| | | VDYVKDNQRFDFKIMSDGVTANIFMMKLNPHESLNPHIDLDTPSRDEYG |
| | | QSTRVMDLPATEQFDRIPAEVIERLEEYDVKIGIDPGAKTMAAWTKQYPN |
| | | LHEECSSIESKTFREMAGFRKHGYNRRSITGRFDRREREAREASDILITSRG |
| | | PHYAQFVQHKLNFCNDGWQAYHHPYVRELKFDAHQEKLAAMYAVAK |
| | | KIIGRSANRWNEALIFFGHWTWKANSPIRGIRTPLKLLRFCLQQLGSTVIVI |
| | | DEFRTSKLCNFCTQTMVNDDYRNSFCVDCQPVERTLAPAPVWRIKAHKS |
| | | YLKRNPGACKIRTFTRDPNQPFRQNNQNHSPMSWNRDYNGSRAILAKGL |
| | | DLVAGRPLPRNYLRTTQRVVQRRRFPQQRR |
| 17 | MDe-13 TnpB/ | MXEEVPVEDKTTRGIKCSLYRIIQNRALREKIQHTAKQVSRITILAALYVH |
| | Fanzor | FRLMEITLSNNPIDLRNEFDEKECHKNRLFENDFYACYENRRNATHIDLTF |
| | | QRFMNAKQIRKITVGQGAAQCVKAAYQDFYRNFKFNITTHAYKRTKKIF |
| | | RCLLKQFPDVLPTQIQAIASDCTHYLFNADSDKVPRRFIVLFFNRFVLPSIL |
| | | NIENHQIYARTKGFMNDSQKTKTWYRFVPNFIRMNHFIIAYNRTLRRGQR |
| | | GMKSFNVIPQYSWKTKHIKIDTEAMFSFKYPLLHYGHGLDLTNKRVVWD |
| | | DIVSRLPKRTNYSFDGSITTNGVSTTVTVRHNSAAKTNKYMKKEQKEAIA |
| | | EANKVAKAAYDRRPKRPNRFFAGKTTNEVPQEIKDRFTHNYYDVLVGID |
| | | PGYRNPAAGVRLHMNQDIERNFKFSIGKYRSLQKEWERDNKRKKITKHF |
| | | DRAEKGSRKRCEEISGVQPSSKDPNYFRINHKLQFFKQVCDAYQHSKISR |
| | | LALDAYIMKWKGITHLAKTISGSHVNTLNKALIFFGDAKVFAANSPIKGH |
| | | VRLSQALLWKFLERYANVVYVNENTTKVCNKCFESFENLKRNPSSEYSF |
| | | CKDCVDRNGVLPSPSRIKTRTHRSYRVKFEYRQEYRIFENFSRAPGHSNG |
| | | KMYNRDAIAGRSMMYKGICLTADLPIEQHFGQ |
| 18 | HMa-1, partial | KMKVVCADPEMSDLLYFGSHNYKNELETFRYTQNQRRLETKNKKYNKII |
| | TnpB/Fanzor | DKVNKETFIDSKTIKELETSLSNLNSKSCNYEKFKLYCIEKNKINNQLYTH |
| | | YEKSFFRKFKLNAFTNTQKSESKMIKNFENKYGKPNKTILVVGDYDKGD |
| | | NNMKGKEPTICKKFRRIFRNAGYKTYLVNEFRTSKLCNCCNEEIEKFLEK |
| | | PSKKPKRKGKIDLCHGILRCKSVKHNCEIYHNRDXNAVQNMLKIVKSIFN |
| | | TGKRPEVFCRSS |
| 19 | SAI-1 TnpB/ | MCVDKLRVAEPLPAAKDFRGQARFTLDASGRYHICVASNFTRGAPDDV |
| | Fanzor | GTLGQSLLQVERPVLAGCDDGLQEVEEEEEARAKCEEHGGGDMAMQAL |
| | | EEKARVRERKMGEVARRLQAEAREARRRWVRLRRAKAIDKQTQEAEAE |
| | | FQAARERHAAHLDWRRRLRKRRRLLRMKERNALLRERRGGVVSIDPGV |
| | | RVPFTLWTASGTVSIAHDAIEELRMLLRNANDIRADADLLEVAVQEGEA |
| | | RGEGDVAAARRRHRQQLRVVRDRVDHLHRVVANYLVMSFRVVLLPRF |
| | | GTSRMIRRSGRPFGRDTARSMGVWAHYRFRQLLLDRAEREPGCTVVICD |
| | | EAWTSKTCGRCGRLHHSLGADDVFACPHDSCRYVAGRDANAARNILLL |
| | | WLYRNDIQLAPVPAP |
| 20 | SA1-2 TnpB/ | MLSILRQHGVGIPDPPKWMTATMAYKLLNLASKEFTPLLQRDLKNKARA |
| | Fanzor | AQRQLKKVAEHHDEGARFACEHTAELALYIGAAQHLARTLAPPDTDLTS |
| | | FKIFPTFTNKHPFIQLDAGLLRYLTCCKCGRLGHSQRQCRQPAAVRPQPR |
| | | QAPSLLDFLGADVARQFRKRRVTSIRTDGYTAYLLVEPSARRDTVCDTSP |
| | | ITPVVGVDPGTRIPLACVRIQDGKKMLVSQKRIANQLYTGRGRRHHKPTA |
| | | TVPARSGGSDEGGEQGGAMGKGETGSRRKKRERGKRTKRERSRRRKEK |
| | | ARIEKEEQLRRQQQEEQGGRDKGEGQEGRDKGEEEREQREREEQGGGA |
| | | EPGSVDDVVASARECLREGVSRRELKWRLHEKLKHCRFRSRVAGELATR |
| | | FGRDCRVFFGDCVHKGRGGGTKAPILELRRAIGCRVDTKIVEEFRTTKYS |
| | | LCCAWPNRIDEQTRIATCTHCGASADRDVAAAKTIAARYGSM |
| 21 | SPu-1 TnpB/ | MPPKKKQKLEKLDKPTCLHTCNKTSFAKAFLPNETYRQRLLDYIAIIHQL |
| | Fanzor | ADHASHALKFYILSAPTFPTVNEDTIEAILYLLNKGEAWHPRKEAKKAW |
| | | RDCLLPYVQRYCQIVGFVHPNLRGEQQSVNYLTASMMTNLKVNVQEHF |
| | | MQMLLRYINLRLDVKGQKQRLPPKSDARKAFFARLRYLKSVFLFDIVPES |
| | | LDDLTAEESELLEEMWTLNLPFPDQDQPLAYLVAIDPLAFFPAYCRLSRL |
| | | YEQHGFRRFSAIPLRRSLIQSHVRIDTVILYPHILCITRREAEAVEKVDLWL |
| | | RVCNLRTKAFRSRRGMQFEGSITTDGTSVSVYLKHPEADKYGKRGARKS |
| | | AKTLEAEVKALYVEKNLPACRAAENVVVIDPNKRDILYCQDSNGTTFRY |
| | | TANQRAMETGSRRFAKERQQMKKEAGIDLIESRIPSHKTMNLMDFTRYL |
| | | LVRRADWDRRKEFYSHPAHTRWKWHAFINRQKSESDLISNMRNKYGEN |
| | | FTIVMGDWSDAGRTARFQTSSKTKGWRTLFKRNRINCFLLDEYKTSSVC |
| | | PRCLASSDDFLEKGFKTRPHSRPWRRREGKIEKVHGLLGCTNPNCLQQA |
| | | WTMRYWNRDTLSTCNMLMIVQSMLDGHGRPEVFSRSVPAVA |
| 22 | SPu-2 TnpB/ | MLSNIYKPISSLMNNRSIQYLKDELPIFPQSSSSTMPPKKKQKVNQPAILHS |
| | Fanzor | CHKTSFEKAFAANDTYRDRVQNYIIVHELAAHATHALKFYILSSPHFLIIN |
| | | ETVVEAMLYLLNKGAAWQPGNMAKKDCRDILVPHIDRYCNVVGFTRPN |
| | | LKYDQQSINYLTTSVITNIKVNVQEHFVKMLVRFINYRLDVKNQQKRLL |
| | | GAERMSFFARLRYLKQLFLLDDIPQTDDLDLSNLESALLEDIWNILLDRVI |
| | | GKEGKLAYLVACDPLSFFPSHCALSRLFERCGLPLFNAIPLRRSFIQSHVRI |
| | | DTMILYQHVLKVSWREYDSVDKMDLWLRVCNLGSKAFTTRSGMAFDG |
| | | SITTDGTSMSVCLKHPDAAKYGRHKHRNSEQAIEADIKSQYIENNLTACR |
| | | SASNVVTIDPNKRDILYCRDLQKRRTFCYTSMQRTKEIGSRRFKKSREAM |
| | | KRENGIDVIESAAPSHKTMNLDSYIHYLSWRHDNDAPLKDFYSHPAHTR |
| | | WRWHTYMNTQRTEAKLISNMRKCFGSSFAVVMGDWSDGGHTQKYQIS |
| | | SKTTGWRNCFKCNRIDCFLLDEYRTSSICPRCLSGDHLQKGFKIRPHSRP |
| | | WQRQKGRSEKVHGLLGCTNPNCIEQGCTYRYWNRDKLSTSNMVTIMKS |
| | | YLDDGVRPAIFSRHVPLVA |
| 23 | SPu-3 TnpB/ | MVAAFSPLTHPDMGCPTCNGTDHHRASSKLCSFYKPRKKNEVRTTLKRK |
| | Fanzor | RDTDLPNDEIFILSTVVKSGQRSPLQVVIAHAARTLTCIAAEATRLFNMFV |
| | | LDLLERKQQIPECSYNKFLHQPFQIIALAKASSPQLQTIHRTLYRQTSRYD |
| | | VTYLSQLISNMAKDYETNCMNHVSTNIRKWAFKWYRMKMQKTYPDLK |
| | | RDKIRILFNKEDDLNEHSLYLKACLMTYDVDITKEVSFDHWWTYFPLMW |
| | | KFQRYFAKHGERNFSMVPITTYGIRHIRLDTNALYKLCLRVDLFTRKDVP |
| | | AAEFRTNALVYWKTLFPDIAETSNKSFAYGVSTDGVACSYYYQKRYVRR |
| | | RQSHNYYGFDDDGKYGAMNLEDGVSIVGVDPGRIDIFTAVDGNENVVR |
| | | MSRKEFYSLTGMVASQKTRNRWLKNNPAVQSIVTQCPTPRTPDIENFRQ |
| | | HCQYVQQHLLTLVDFYGARRFTRQKLHTYIGKQKVWATIVRRITKNDRR |
| | | TVVALGDAKFRHNSKGGPSTPLARMAKELKRRHRVRMIDEFRTSITCSH |
| | | CDGELPKSTRHWQVKVRWAVMNSVSQKXSXICVFCQVCNDLCLTHWN |
| | | RDVNAARNIRNIFLHMNENNGERPCCFARTVPQFNPGWNL |
| 24 | ROr-1 TnpB/ | MDRNVTDHKIDIIIDSFELYVEDNSDDDFQPVVTARKRKSRENIPQSGSQK |
| | Fanzor | RLKGKGKATDSEVTTTSSTGISTTDEPTVFNKITLEDYDLEYSSDDFDAPV |
| | | IKRPFLGESTKSASRITKSTSAAVESSTTASKTSINSPKPSKPVPTAFKSAAT |
| | | SVKSSFCPVPVDKASDIKEGPKKISKRVMTIKTTVKNIWKPAYLQPLYDL |
| | | VHTTNLLVTHTFAFTKYIYLQELAANENFALNNFVTKDFFVEVFLSLVLS |
| | | KGGNSTRLKDTTKNYRRLISKYKEAYFEDAGYTPPNLPYAQQIALYECT |
| | | KIQTAYYNNIKAHFGNRLRSLINKLFKKKEKAESLRGEMQANKSSSKAIK |
| | | EAIRKKIYRPCNQVKLAIAKKEMPEVGLLDDQSRTQLNDFLSSYPEDYTF |
| | | QENSIYYDVMASPKNHFKAFFRLAELSEAEQTKQFACFPLRSTFIPCYMT |
| | | LDSKIIHYHILKSKKNPKTGSKFETWGAVVDLNKKAFKHQGFQKSLRFQ |
| | | GTLETDGVGVSIIKQNTDTSRKSPKPNTEKKVDGNQTEHIEGLGQADLKS |
| | | TEGKCVLIDPGRRDLMYCMKETSTVEEKQTLIFTKNNRSKCSRHFRYLRK |
| | | RTQPFVVQKAEAILSRSESNSVNLKKFVQYIKTRASVKNTLYEYYGNETT |
| | | KSKETYFPESEFDFRVDQKCNLYYGNLFIARIRGFFPQPENYSTDSSVKSQ |
| | | LYATYLQIMLQQKHISERLDDSEKSKVLGLAKEMYRRPQKSNYKKTIST |
| | | ALEKLQLLPFRKLKFSSKLFFDQNDQKLVRSLRAKFGQDAVLVFGDWSA |
| | | PNVKYQEPTRSKGLIRMLKKNGFVVYLINEYKTSSHCPTCENELEKFKTV |
| | | PNPRPYQRKKKPDVLCNGLLSTRCKSLSCLKQQTTGIKRRLWNRDQAAV |
| | | LNFLKILNSLRKTGERPSLFTRKYPLD |
| 25 | ROr-2 TnpB/ | MNEDSLPSTSAGTMSTTGTLASGDRILSVKSSIDGIGWNEQYRERLEEFV |
| | Fanzor | NIIHATTTHAYSLSKFIFLCALQDDANFDIASYINKEFFSEVWLSLVRYRR |
| | | GRVGEATARRRTFIGQYIQRYLDYTTYERPELKYAQQSSSVEGLKIYTAY |
| | | TNNIGAHFGNHFRRAINTLLQIRQRKADLIRQRQEEGVDNEAISNEVYEQI |
| | | TLPARQFKEILASRIVDPSSIELPFQDPIYNQALQRLTPILTSYPVRYTFEQN |
| | | NIYYDAKANPCEHMMAFYQLARLFEALRLPIFNCFPLRRTWIPGYATIDS |
| | | KILCQNILGRRWTNGINKMNLWAEIIDMNSAALKPQELGVLRFRGTIQTD |
| | | GVGVTVLKKRQDRKHRYIGLQSTFVEPEPYITDLNAQQHGEITGRCVTID |
| | | PEDETFCTVYMKTLLLMHHAHIDTPSLVKIRWKKTKKYRRICEAVKPQE |
| | | VQNAEDTLVNSRSLDLQNFEEYLRNRALVTELLQRHYTETTTNHLTTHP |
| | | LHRKLKLSKYIRRQKASEDMVAKLKSKFGNDAVFVMGNYSAPNTRYQE |
| | | PVRGVGFRRLLKKHGFLVYLIDEFRTSQCCPSCEHRSLTTFKRIPNPRPYQ |
| | | RRNNPEVICHGLLRCTNQNCKVTVQNISGAEELRERLWNRDLAACLNMI |
| | | HIVRNLRLNGEIPERFQRAGAERRRSEENEERRVLPRTL |
| 26 | ROr-3 TnpB/ | MLSKDNDSTNDNSNKKCCQFIFFFFFMEPPLKIITVKSSIAGIGWKPQYIPL |
| | Fanzor | LKTLVANVHTLVSHTFSFLKYIFIHELENNPAFPLENFINVDFFKEVFLSLL |
| | | NNYKPDKQKASTKSRTYKGLVLNYRHQYFQCCSYEPVNMKYANQIASY |
| | | EVVKIVTAYLNGVKVQFGNKVRMFLNLLLKKNERIKALKSEMKKNGGT |
| | | EKEIAATIKTITEQISKVKLAISSRNIEDMPKEFFSSNDLDKIRNLFDSYSTD |
| | | YRFAKGSIYYDCKANPLKHIKAYYRLSSMCEALQNKSFNCFPLRKGFIPS |
| | | YMTIDTYILNTQILKNSIISHLDKEVVWGAVLDVTSKAMKPQGERKFMKF |
| | | RGTIYTDGVGVSVLKQNYDTKKKGGSSGGKPKSIEADEFQYIEKLGKED |
| | | LLAGVGKCVLIDPGRRDLLCCMHEKSTVENKMIYRYTSNQKAIETKSRK |
| | | FRKLRNNLKRDEVIAAELSLSHFKSSTVNKDKFVEYLQERAKVTPVMKA |
| | | YYLNEDRPAAEDQGAGGFLPFRKMKLSSFINQQQADKRLAKKLRERFGN |
| | | DAILILGNWSAGNVKYHEPIRGVGMRRMLAKEGFQVYLLDEFRTSSLCP |
| | | SCQNGELETFKKVQNPRPYQREKYPIADRHGLLRCKNQQCLKAVTSTIEA |
| | | TXKVPLRRLWNRDMAATLNFRHILFSLRANGERPERFCRSKKPLSTGSKR |
| | | KNMSSSSASTSRPTKRPNNPLX |
| 27 | ROr-4 TnpB/ | MNRTKAKQSSRFVGTSTLPPRRKHHSNSKRPRSPSPGEGTSDNKRKRIQF |
| | Fanzor | SDTELDAFFFLSMENKLKESIRTVVRKNNKKMNEQLAALKQKLSTAKKL |
| | | PSGIITGAAKLAESTYNEPSQADLILRQMRSFMDNRVGKKNEIEWQEFVV |
| | | KKFDTFKNEETNLAKSAQPESNDTDRDSVPDADCPAVDENSYRTCTASIN |
| | | KLIREDLDPSIKDLIMKKLQRAMIGSTDYILYFSSLIQMMMICLKTNQFFL |
| | | DGDVINTRKVNGFDMTTIVPSEFHTDSPKYTITPLAMDVSDSPAFDADFS |
| | | QLFQEQHFSLISTKYFGNRGSQDQTLNAHPIQTALFKLMDAEDIKKETFP |
| | | VENVPSSLMSLALQIYVTNFKNMWADKTMINKLLDKLLVTLLRLHLAPK |
| | | RELQRRQNILNYISRKPEEKRPSKNYGRYILRAEKKKLARYKEKKQDASS |
| | | SSREKSVESTTTTKKETTPCDDLSRGRISQLKSGAKHLIFSKDLVTEKDVN |
| | | DEFPDATRKEVSVCLLIINTLNKFVPVRSKYHILPYQLLFCIFANDVLHYA |
| | | EYSKFTRKLCPAMSSAHVNALNIDAPSLFQIMKSKTDDNQGQVEGMSTE |
| | | LTIYGYNEEPLSSVEIARRNKDAVFNSLFDMEKVRHTCESNGLKFGQRMS |
| | | VLPKLKVVRILGVKKTTVPPVKPQKMSYLKRILRDPNILREGQKPLEDLK |
| | | AETAKLEKEVKSLDQEREKALKCQKEQDSIKKIKALRKEWHNKVDKQT |
| | | LYQRIEEERHLRDKTYLKINDIEDQLMEKKQVLYYQRMAIRHNSRIHTVD |
| | | TASAPPTTKKELKKDSRGLVHSEACVLRDFEDFTFCGTDNGLVNMTAAV |
| | | PFSLKRFSFHLKLHNYYSVLDDDNKAAPVVQDIELQKYLKTPKATTISAS |
| | | EIDFGCGAFKQRVKRQTGKKLTRKGKEVQLIENSLSQKVTSTSNVSVEQF |
| | | KHNFDAHVEHRKKLRDFYNTGNRMNERRHREIQKHRFTHRICTKERKAL |
| | | SQTSGNNPSTKKKLVMFIGDRGTGVGSRIKGFKRYGGRWKEVIHSEAAN |
| | | VCVTNENLTSKTCIFCFRRLTHPRISVTKNGEVFSRKVKGSFYCTNPLCVS |
| | | VLSKCAAKSRDSLSALAIGIVGLSTILFGAPLPAFKSHKISNIEAEKYIILTS |
| | | PSRFEEETGSYAVVLE |
| 28 | AMa-1 TnpB/ | MADTTLPVDAVPSDAAPVAEPILPAPVEDVAPGLAPSVPALSAVAPSDQA |
| | Fanzor | RAPIVSVVPQTLHHALVDAPEYLAVRAVIEARSERLVNAHGFAATEATL |
| | | YLRYRARRAYEDFRDALLYAEEDYESSNDMVSLLKAVTVADQQADYLV |
| | | FGIPNCDQLDQYATEYLHHVHYPNTPFVNRSFFTRSFEYLFQEIVKNGSE |
| | | HFHRTFRYAAGRCINPFAVLVESWPPPAPPRDQRAVEPPANRRMRRDFY |
| | | AAIDKWVTTICRRGSKLQLDKFLYKCACIIVDYNLFILQNPEYARATSGL |
| | | VPDLGVLHVPVPFVPPPPAAADDEAQGQVEQQDSVHLPRFIPEGVRIAAE |
| | | TLPRNRHARRKQGKAWIAPAVADHFAVARRWFCPDLTIFPHMALKRRH |
| | | VRLDSMDITRGLLAEFPGNSPDLLHVVFGLIMKHAHLDSLVPGASDLNVP |
| | | DEQYELALEQYAWQKLRDISPQLARLRTKFCSDPSISPVLSGYWQKPRVD |
| | | GAPRVGSSFTTNGYAAHILISRVPTDDDVRRVGGEGEIKQKSTRRVVTRG |
| | | PGRAVAAKSLHSRLGTGEFAAIKSRSLIVSDYPKGLIAVDPGQSTLMTKT |
| | | TLTPFRVGAFANGWINIKWEITRRRNPGYKSWSIPGPRWSAATGQRNQTL |
| | | QILRLRQHQSDLEQIESRLPAVHDPTLAGIIVLAKAVTTNMERILGFKMDR |
| | | RYAKYDLQATIAKQRALAYLRNKFTGYGVLFGNKGAVQINRVTRMPVK |
| | | GVIRYLLVYGIDVLLVDEHRTSKFCAVCGSEFEATPAMKVNRKFAYRYL |
| | | HCPSCKYVVNRDVNASWNMFRVALRDVFDIYVADSMLKHPLALLPSRG |
| | | APRPLLPQPGDDDFDDSAHDRYSGNHGGPGG |
| 29 | AMa-2 TnpB/ | MGLVRHFTSAVTHGDVRIMFDVAYPLKDRGGDMRIIYRIVIVASTHDCQ |
| | Fanzor | VAVAGRTSCVFDLATRSQASVVLAVPSSWIVSVLVRLPGKAMAQEMPW |
| | | QALVSLDIIFGLLSEIRIAKFGHGVTSAWIKRILTVSGIAKGLIEVKSGNIED |
| | | DLLHLSDLSLPAARMFTVQAMSNDYWEIELPDLPSCHYLTISNSALHFVT |
| | | DMMLKGFLDLTSHLDSMAIEQPIALPDPVLDQVALTDFPHRFRSIKRLHIG |
| | | VTAYGWFVSLDGDHEAVHMTDLCLGNDYGHGVCCQRPGCTEWLHKCK |
| | | AAAQIQCHTHLEHNACLDPVGQTVTPNWSRVLTSSIHWPLSYLMKPGEF |
| | | ELNHCQLQAMLNNQLHTVPEVGANAAAIAASRRDAAQHNQLLETYVKK |
| | | SQHTQAPPNQTGKWRSMSKGMMQEKRGRVERSQFMDKFRSARPALRRF |
| | | NANMPMFKTLDLAALHNACQYVTAWLPVVKPMALSNSLASYEFGAAN |
| | | AERAFFDRLAAPLMLSPDPTRPALVYFGNVGHLSPHPHGVSTPVRKFLC |
| | | MLVHAARVVLLDEWKTRKTCSLCGSEMPNDACRWGAKRCRTCKRIRA |
| | | RDRNAYLNMVRVAVMDVLAVSFPQHWRDRGHPLCKRAPLVISLGGAQ |
| | | QVGAGGEGGGAA |
| 30 | AMa-3 TnpB/ | MHVCMQLMLKLMHLPPKFHNQDAKKMQDVMLVTTLELAEALATGRV |
| | Fanzor | MAEQAVVAFGHQLLATHMQTNCLTDVFLKEVLARVQHLDATYQVTGK |
| | | PVGKLHSLLISIKDMNCMAGTDMALSYSAFINQPSQSNVLMVDIVLCEGA |
| | | NIIAKTNFLQMMLLFECSNPVFGHTANPYNLNFMLGCTPDAMEPSTVHTP |
| | | DESSTVHWHVYTQWVQCPHPHVIHTACKTAATIKVTGHTLVPFTVLALL |
| | | KLMQITYNLLLQDNAHATMVLVEAAKEPIELSAIKLVQLLQLPQWVWN |
| | | VLVRVMSWLGEMCTVMFLNAMCIKLNHIVPKRLLLQYLDAAANQLLV |
| | | MWHIMPVPTPQAATMSWQWLGSTTCLRVRTCTACSHMSCTTMRLSTM |
| | | GWARSVLKSCMPWSLSCKMCSYNLLVEVVLDLNMVHWHADMIDAVL |
| | | GEAPEHVDLANLLMPKDWFDWAMAENVAQGNNYLARQLVNNARKLR |
| | | DEVRACGVAAMLKDKYQKTAPNWSEHGPIVGVESRRQGYEQRNLDPD |
| | | DFGMFRFVDPGHKSVVTVLELNHNELQAVIDNPLRAMPEDGADAEAIAA |
| | | SESDAAQHNRLLEAYCPHSQRDNSHPRYEQKFWSISGQQLREKQGKTQC |
| | | SRHQFRAANADRAFYDRLAARFMDSPDPTRPALVLMGNSGHFSLRGVS |
| | | APIRHMVRYLIHAGARVVLLDESWSSKKCSLCESDMPNDRRRWSSKRCR |
| | | TCKRIRTRDRNARVNLIRIAVRDALDVPFRQHWRDRGHPLCERRPHANG |
| | | VPIGDDGGLDQGGGGGGGGGAV |
| 31 | PB1-1 TnpB/ | MNEATPFRALTVKASIKATGWSPEFLRPLQVLVAKFHTIVTHTFALSKHIF |
| | Fanzor | LAELALDPFFDLNGQINQPFFVEVFLSLIDRNTDSSRITEITRLNCELISRHI |
| | | NTYIHNSSYTRISLLNAHQIAMYEATKIHTAYIVNIKLRFGQHLRRVINILL |
| | | DIKNRQRELTQQLRSQRATEERIKRMLTEHIFEPARMFKEGVSTRRLEDV |
| | | FIEEGYRTAFMEISPILTAYPKRYKFQKDSIYYDSKASPHNHFLAFFRMAQ |
| | | LLEHLGRRSFNCFPLRRSWSPGYMQIDTRILDTIQQLLRRTWSEGADKLE |
| | | LWGIVVDLDEKAFRDQQNGVMKFRGTIQTDGVGISIIKQNKDTTQGPNP |
| | | RRATTINPENIPYIHDLSPEQHERIRGRCVLIDPGRRDLLYCMHEESTVETP |
| | | RCYRYTSNQQNIRLKRTKFRRILQDLKAQAPNVIAAEHEISQTSGRPNNTT |
| | | DFAFHTSVKAANSSILSSFYTNTVSRTSQKPLFRKLRLSSYINQRKANERL |
| | | AKYIRAKFGQDCVLVMGNWSAPHNRYHEPIKGVGMRKLLRKHGFDVCL |
| | | IDEYATSRHCPRCYQPSLQTFKQVDNPRPYRRTQMPTVTRHGLLRCTNL |
| | | NCLENTDGSEVMHRLWNRDVAAVLNFRHILNNLRYDGTIPVRFTRVIQI |
| | | GRIRRQAEEDLQEGRRLRQRLTRIQRR |
| 32 | PB1-2 TnpB/ | MELPLKLTVVKGSITSIGXKLQYVPRHMTLVANFHPLLSHTFTFLKYIFLH |
| | Fanzor | KLEDEPNFPLENFVNEEFFKKVFLSLLKNYKPTKQKASAKSIAYKDLIFSY |
| | | RDQYFWCCSYEPIDMKYAHQIASYETAKILTAYLNGIKLQFGNKIRMFLN |
| | | LLLKKNERIKAMESGMKNNGENKEDIATAVKTIIDSINKIKQVISSRNIEHI |
| | | PGEFISSATLDMIRNLFDSYPPDYQYSKGPIYYDSKANPLKHMKAYYRLSI |
| | | MCEVLQDKTFSCFPLQRGLIPCYMTIDTRILNTLILNYSVISHLNKEVVWG |
| | | SVMDINSKAIRPQGEAKDVKFRGMIYTDGIDVSVLKQNSETKNKGVGGK |
| | | KSISLVENEFRYVEALEEEELQAGIGKCVFDDPGHRNLLYLMHKGMTSK |
| | | RKSTYRYTRNQKYVGTKAKKYRKLRNDMKLGHIIAAELFLSQHNSSTTS |
| | | KEKFINYLQEKAKATPAMKAYYANEDNPAEEDQRISGLLPFRKMKLSNFI |
| | | NQLQADKRLAKNIRKKFGNDAVLILGNWSAGQSKYHEPIRGIGMRRILA |
| | | KKDFQVYLIDEFKTSSLCPACQNGELETFKKVQNPRPFQRRKYPTVDWH |
| | | DLLRCKNQQCLKPVTESTEDNTQSSLYRLWNRDMAATLNFRHILFDLRK |
| | | NRRRPERFCRLSARSFTTLKRKRSSSQ |
| 33 | PB1-3 TnpB/ | MRRKKTPQETFYQGVNTLPPSFLQSNTKRTLSESFSEESFNKRNRVEGPV |
| | Fanzor | KDLEECFFTSMRDRVEELVKQTKTQDKTNELKNKLLNVKTLPAAIITNA |
| | | AQLAKCDRKVMRSHMEHRPGLKNVEEWKKFVSQNFDKIAEAAVGSVV |
| | | KDTAKVYSSSTPTNKQPYEEMRTCTVQINSLFRSDLPPIVKTFVCTRLQDS |
| | | MVTSTDYTLCFSALVNMMISELRTSEFFFDNNDIKIKKVPGFNLAELLPFV |
| | | TTNEPKQTIQPLDKDLIASKRFDTDFKCLFTSQHLQVVHSYFFGARGAKE |
| | | ENLNAHPVQNSLFCSFKESGLDKQSFCLEKASSSAMSMALETYLVNFEN |
| | | MWDGKKIINKLLDKVVLVLLRHHLARNRESKRISTTTTRNPPGKKDMRN |
| | | HARYVCRAEDKKLKKLVQRKEKAGGLEGEKWATKINSAKQRLANLRH |
| | | TFKKKISQMLNDRKEASVEHKLVVQDLNIAEEQQDFLEEEGTLDDDVPE |
| | | RRLNQLKSVIKHLVFSNDTPVYLEDVKHQSPEATTTEQSVCLLICNTLMK |
| | | FLPPKKQYHVIAYQMYFCIFANDVLKYARYTKFTRALCPSTSFSSSSALH |
| | | LDSVALYQLLTQNIDQEKSEEPPSHTNQQEKKGYSRMILYGYNRDELIGS |
| | | QDKARQNKDATFNAVFDMGEIQKACESYGLSFAHRMTCLPGMKTVRLL |
| | | GSKIKTHGTVKEGTKQSYEARILRNPSIMQEGRKTKDVLFSELQSLTEEV |
| | | KTLESVRKRELDLLKDSNFQRKIKECKSNWGTTDDKDQLYRTIEKYKES |
| | | RYKSYLTVNKIRNELAEKRQQLYFRQMAIRFKSKLHNVKDVPQDSRSPIE |
| | | KCGKGVTKAEDRTVVNPGDFNYAGTDNGLVNMTASIPMSLQRMKFHLK |
| | | LFNYYTALSKVSNEDSIGLNLSKEEESFLHLPSVTNTKASDVDVGCGYFR |
| | | QRKYLERRKKYTDEGKKVQLIEDSMREMESAPVTSIDMAIRNFRAKYDQ |
| | | RRSLRDFYNSPKIINQKRHVEIQQHRYRHHLCRRERLELKHSENKSSSKKP |
| | | LILFIGDRGTGAGSRIKGFRKYGGKWKQKIHGEAVNVCITNECKTSQTCIF |
| | | CFSPLTNPRIPGKKEGSYKVNKGTFLCINPRCITVKNRCASKPRDALSALA |
| | | IGLVGLSSVMFGAAPPPFYNVSQITAEHYTKITSDFCTRRDDLAATL |
| 34 | MCi-1 TnpB/ | MDDADFPGGNGDQQGAIDAMRGERITVVKGSIASINIRRPVYRDRLEHY |
| | Fanzor | VNLHHKTTMHTYRLLKYIILHRINNHHFDAMYYLNHRFIHEVYMKLITK |
| | | ARERAPRTQDTIERRAIIDQYLPAYLRLVGINDPRELPLMXNSQQSAMYE |
| | | AAKISTAYLNNVRNNFGKRLRQVINVLLNVKARQRALRQLLRGQAMDQ |
| | | RAINQAIRRQITNPARRFKIALSNRTTIEALHARFDDGPEGFYTTAIDQLAP |
| | | FLETYPNNMQFAQDNIYYDCKANPHLHFKAFFRLAELLHQRQVRSFCVF |
| | | PLRQSLIPGYVIVDTKILMTQIFQRSVRPGEPLRHRHEWGQFIDFRMPIFRA |
| | | QAGREFGNMIETDGVGVSVLKREQHDLQFQQPRQQGAPQQQEFPYITDP |
| | | EVQIPPNCVVIDPGRRDMLYCMEENSTPQAPRMFRFTKPMQDKIRKNKR |
| | | YRRILQQMKPRRIADMERELTNSNTLNLQVYQQYLQNFGRVYEALLLYY |
| | | SITRGASQTGQFPIHRKLRLSAVINKNRCDQFLIRFLNTKFPNTTTYIMGN |
| | | WSAPHTRFQEPIRGLGFRRLLQKHGKQVFLVDEFKTSKVCPQCQQPTLET |
| | | FKQGINPRPYRRATQLYTTVHGLLRCTNRNHAAHGLPQRFPFRYFNRDT |
| | | AACLNFIRCLQAVRQGLPRPLDLQRRPLQDNDDDDEEDDD |
| 35 | MCi-2 TnpB/ | MHSRAIGMVHSNPKRLSLSKKRPSTQQASFPKPVLPTSSLSAQAPSAEVIS |
| | Fanzor | TKSGGINVPSSKWSPKRVSGGEARASSESAGSSTGVTASSSRSTSKVADL |
| | | AKVSQTGGAHVSDDGSSSSRVPPDQSTVSGGGGEIASSSTSGNQESPSPD |
| | | AVALRVPFTVECDLLIDTNDLLPQAAVSGIQKAFDDAQSIGFNESINNCTK |
| | | FLASKHVVIDGGHRTLCNVLNGVSRTECKIHDNQGGSQKPKDMLLLYCL |
| | | ASHLQCAVYVFSTRHRPVLVSPSAQEQVQGYMALLVHKDSYMQKTHW |
| | | YPLRLDLKSKVGSARAKTTLETPIVATRKQSIAQASTVSSSKPIAKGYEKD |
| | | IEDILRASCFSIFRKKWDDFRSSGNTSIQDFKATLSVKSFPENVVQRCSDAI |
| | | ENKMRAAGTPLSRNPQWLSRKLHSWKTRKKNPFGVVQLVEEFIAIITKE |
| | | QNKELADQGDDDPIEVEDGTSQDQDDPIEVENDISQDLDDPIEIEDDTAQ |
| | | VQGQLVPIKEYHTISRSLKSVIKPQADYQTLLLRMTNLQNTCHHVVYGLS |
| | | QCVSTLVNMIIEGECSAGAHPGYLDFSRIDLHQRGIGAGAEQAISLVAPIK |
| | | QDVLDLFNFAGFYKIACAVVGQKNLTAHSADFPLPANDRVTFVCGPWV |
| | | QDACRRFLTNFINMWSPASCVADLRVVMEMLLRIHLCSQRFFNPKVRPK |
| | | KTKKQNKVKIKAPSKDYAMSKLFWALVHKDPKQHVVDFFTTKVFKLFP |
| | | TPKDITTAATVSASASVATAMNPYEKALQDGTDASEDEEEEDGILLAQTP |
| | | DLPAKDIRALTSVACFLCQSPEHPTVITMQHVKEKLYHDKQDCIPSSALE |
| | | TTASLVSAVRSVTPKEDQAFSLANWIPMRALRNTLVEFAGSKTKRRVSK |
| | | LCVDKKKDTNSADAVRIDHAALYHLFSNDHNLHLPNGIDKVTSVHVLKT |
| | | TEQKVDLLNNFLKINKIKGLFQERKLTFAGYFLFSNQYDLHFAGYKIVQP |
| | | VKQADIATRGRQERSGNQNHEAEKQLVKELTKEIKDMRKTYTANAKEL |
| | | RKLELARMQAGNKSRTFERRNKRRDPNLYRDLKDARDTFNKLHAKQAK |
| | | LLQEIQERQSRMYILNKKIHNQRHPPQGSHLDPKLAPIVAASSTVVQGIDP |
| | | GIVTAASGVGTYSRSLFEAVNRFQAISNEEEVPHPKDQAYPFSLTANMVN |
| | | TAVLSHQHRKQREKKDIGIKKQQRNTVIRRCRLKKYHQRKVASDNAVY |
| | | HQPKGAHIVTFTGNWSGSGAYIKGHARRSTKLYYSQLGAGTNHSVVSVD |
| | | EYKSTVTCSSCFHRTSKQVHLRDGHIKRIPGAVVCLNVKCPRRLTSGATT |
| | | VNRDLNGAKNIALIGFSSLAAQDGLALPPFRRSHNTN |
| 36 | MCi-3 TnpB/ | MSMEDTSAIFDITCASPATLQGNTDLAFYDTMIAALTNWGPLGCKESFKH |
| | Fanzor | TAKFLRSWGVILNENGSDLCSIMNGTVKHNMRIKSEQHPESVSPKGFLLL |
| | | YCLAEHFNCTVFLFSTRKKPTIIKPMQGDRATRNFAALLCHQDSFMQKTT |
| | | WFSLKRQDGFIRNSSALKAPSSLANANNDIVATKFVKDSKKARHETIYSN |
| | | EKSDQQVMKHLQEEVFSVYRKRWCEFLQKKQSSKSVDDFRKTLSTKSLP |
| | | NKVVSKVQVQMEQQLGNIQPGWVQAVIQRLRTAKTMPTAAQYVALFIK |
| | | DLKKELETAQNGEPSTSGTSASGTSASGAITAGASKAGASASSGTSASGA |
| | | STAGSSTAGSSTAGSSTAGASTAGASASSGASTGPEDGAGGAGTGGSGSD |
| | | QVIRSVSRSLKAVLHPSFDSKVFFEVIKNFQASCHLCIRGLSKAIGVLIDFV |
| | | LRGQLGGKTKATFFNVEDVDLFNCGLGKKAKPISFINDTDAGALQGLFD |
| | | YSGFWKTLSFCIGRKNEDKTKIPKCRLLVRIQEKLDRTDYVDFTTRSALQ |
| | | EACRMYYTNFSNIYSQKNCQAVLRTVLTLLLKINLCRERLFGDKKTRTY |
| | | KRKQKKGPKPQSQQATKKLAMDKLFWLLGSDCQVDQCIDKQMRIIFSV |
| | | NKKKPNSKQLAAPSTPITPSSVSSKGKSPMQATSSSCSLSTANDDPNIELV |
| | | DNDDSDLESVIDDDIQDEEEYRDLTAKEINALTSVSMMLCQSTTIQGNVS |
| | | AQYVKQQLYTDKRDSIPAIAIDHCCAIINAIKSLVPKEDYPPSYKWFGLFE |
| | | LHNTLVTFHQGITRKNPRALFPTVDNPHNSQDRMAIALDAATLYSLFCKD |
| | | YRIYQKDKSEFTSTGMIKTTQAKEDLFSNFFNMPKIHKILREQKLTFCHYI |
| | | KFKDLYNVHLVGVKTLGSSSSSGIKSTIDKPDPKPHVPKSSRSSSKADVIK |
| | | ANTLSSTLKETKKKLRPLKLQLSKLQRSRMDAGHASRSWEKTNKALDW |
| | | DLYSKLKEARKKHDTLWATVSSMKAQIKQMSSDLYILNKRIHGQQEPQA |
| | | ADAQTPQLTIDENTVIQGTDPGVVTTASSVWCSSRTLFASINRYQVLWDD |
| | | HDTKPPPADQKQIVKSELTAKKIDTAVLSPRRQKRTRSSGKTLQKARATR |
| | | RIRLKKCHQHFVAAEAKSADIFSTGQKPRRMVPFIGNWHRNATHLRGHC |
| | | TRSMKPYLSNLSTTSTVAFVDEFNSTKICWACFHETQKQLMRNQEGHMT |
| | | RNLGAVTCYNTKCPMRASKQTTSNRDAQGAINIALIGFSKLVSKDGSVLP |
| | | PFARKTNHDK |
| 37 | MCi-4 TnpB/ | MSRRSKPVQQQTTFTGVNNELPQLRSRESLKRQISDVPEGPSSKRTRVHS |
| | Fanzor | LRDYFNQSMVNHLNSMVQNAWKKSKGDKAKFTKEVNLLKAKSETPRL |
| | | PSGTLKDAANLAHCEHHEMITYMDNNGEKLNKFLWKDFFDANVDNVA |
| | | QRFQAGHAATSSSTPAGPSAAPLQPMAAGPSSAPFQPMAAGPSTPSMPFT |
| | | SPFQSTAAGPSTATASSDDDDTSSETTISEIGSVFDEDPTKDYRTCSVTMN |
| | | KILRADLPEAIKNIIFGKLNDATHRSTDYALNYYLVLHLLLLKFRDGTFAL |
| | | ENQTPVFRDAPGFAIARLLPAGYVKSEHCNVTHSALPIPTAFFASTNLAQ |
| | | DFDLLFQKSHLALMQSYFFGVRGALESSLTAHPVQKALFQSLIPCNVTPQ |
| | | KYSAHGLSSAECQNVANKIQTNIGNMWKGNKIFKHLLNKLLHALLSIHL |
| | | APDREAQRQEATTKRDKGKSKDKGKGKERATPLPPSAGGPSTTQSASIKP |
| | | FTRNAFRNIRRGETKKINSYQAKKEQDPSNPKWDSKIQHCQSRIDRCYHL |
| | | YKNSSDLSRRRLRVLTAVCRDLLLDDGIAACTEDHIKKLLEDPQQPEVDC |
| | | CLNLVTRLKPYIPRKDAQNSLARVLPFCLLANDTLGFTGYNKFQRKLLPD |
| | | PQFTHQYPLSLSAQTIYYALDRGPDRLIMYSYDGSVLISATKANQNKDAC |
| | | YNSIFDLNAVQQVCKSYGLTFAQHMTLMPGLKTVRILGCRPKKATYNED |
| | | TKGDKQQRWTKEVLQNEIVVREAQKDDDAINADINEAEERVKVLKEEL |
| | | KSALKEFDYASFGQKIKDKKKTWIPGVTNNALYGEIQQLKIDRNEAFMK |
| | | TQHIRQEMRSVRQSLFYMRTAKKNKSKIRELNAAKSTATTTAEDIPALRR |
| | | GGHGVHIGPRRLAQDIDIDNDVVFSGTDNGFVTMTETVAFDAKRFAFHL |
| | | DMFCHQGLDKEEQAALVAKHDPAFLSLPPSYKIKADDVDFRCGYRKAR |
| | | LKLERRKRENPHIRQLEQASRESSLKGADTAQSYLSRFSVQQSNSGALQA |
| | | FYDSPARRAENRHLELHNKTQRNKICASERQFVAQHKKEESSGPAGEGSS |
| | | RRKPKPKPIMMIGDSGLGVGSTIKGYRRYGGTWKQNLQGGNTHVCCTSE |
| | | YMTSQTCMYCFSKLTHPTSKQVVKGKVVTKKVPGAFVCLNPDCVLQQF |
| | | GRNVFARDKLSALAIGLSGVNQLLFQATFPQLSRKFSDTSTDFINKTASFR |
| | | SGREARASV |
| 38 | MCi-5 TnpB/ | MFSRRLIESSLMAIKRKRRYQETTFTGVVNNPPKFRPAGQRLAERAAVGR |
| | Fanzor | TKRRSGDLTEDEAWRFFDDGLKDVIRSQILGARLDERSIHDVRQKLVSST |
| | | RILRNVYSNAAKIHDLNAKAVTDKLRYKNTVKHQQEYIEDHFEEIANKP |
| | | DSFPKVRPQPKKDIPGAPAGKQQRTATAPSSDLRTVTTTFSSIIRSQGITNN |
| | | MAQHFREIFNAATEDVSNHIIDFSVQVLRYITMFKTHTFVIRNDQVQSVPT |
| | | VAPFTLAEILPETIFDEATLERPLPPPLDQGFLNNAPLLEQARNFLNDQHL |
| | | DRIFTAYFGRTPALQVLQRYPLHLAVLQTLPPPAEAPPIAFSHAKIVARNK |
| | | FKTNFENLWNLKGRFTKVFNKLITTLLRFHLAPEREAKYQEYLTSRTQDQ |
| | | GSSTPTPAQAPHGEITFAGKTTNEIRRIFHHERRNQQNYTDKAKTEGARW |
| | | VQKAQRSAHRLATFTAVRLAENGDLSARAIRVLVAAMRNVIFTLHGQA |
| | | GQVTRAQFRRIAGNTINGNTLEVCLNIVNRLMPYIPSRQNWFSKLHQIPFI |
| | | LLANEVFRATGHAGFAVKLTNLPSTSSLLALELDGPSLYNTFSVQQGDRR |
| | | VLNLFTYDGRIITEDVTARSNDDAIFHSFFDLEKINNILAPYQLQFRHQITL |
| | | LPGLKTVRLLGSKRDNATVAHAALPFVEKKATAPSKPPPAREQPTDAEK |
| | | REIERLIALRTAEVEALARRLKQTMRERAAFLTNEYPYVKKLKATFPNQH |
| | | PQQQQQAQQQQTQQQQQQAQQTQDFPLTFRATRNEMYQRIRQLKLRRD |
| | | EFNQQAAAIKHNLAIQRSTLHRLRKGLSIDPPETREAPPTPNIQITQDRYQC |
| | | YEKAENVRLHPALHNSQNMRFSGTDNGIVTMTETVRFDMPRFRFHLQLA |
| | | NTFEALKDLDGKQVIDGCNIARLTFIHAETEAATRYTEANPDLLRVPKAH |
| | | KMYATDIDFRTGAKKQRARLYREKHSPQGLPVVQAEAILSQNSLHDAQN |
| | | LDEINARYQAQLGERNTIRHFYYTPAKARQQRQYEYCNRKQIDRLCSEE |
| | | RKYVKSDDGNYNRERDRHHQTIHFIGDRGTGVGSRIKGHLRYGGHWKS |
| | | RKHSLNTSVCITNEHNSSQACAFCFDKVTHPVKMVKGELKTINGTAVCS |
| | | NPRCLLVINKRSHQPRDSQSSLLIGLAGLCTVLSARTTFPALDPKNQDISQ |
| | | SNTDFQNFAGSFL |
| 39 | MCi-6 TnpB/ | MSSLRTKVEQKTPFTGTETSRPPQRKAVGKGKKREHEAASTTDTSSSAQP |
| | Fanzor | LFKRPKSLGISSQRLMDSFFQSMRDHIIRKARPVIGSKIKIDALKTQLLAVK |
| | | RLPPGIIGDAEKICQCAKNELKNHLEYHGTMQNFQYFQKYVKEHFDAIAL |
| | | SLQPQASTSASAGESASTPSTPVMAPADSSVALLRRDLSAVDMASSSSSA |
| | | SGAPSQAALSSMSHAPSDISMVDDLPEDEDYRTCTVSFKSLLRTDVPPNL |
| | | VESFLQKLNAAMYDVSDYIMGMQLVVFTLMVSMKSLQLVSRGGDGGV |
| | | QMKKARGFQIHDILPADYEMKIDAATSAPPPLTSSALKSATFCNGIDDLF |
| | | QGKHIQYLHSSLFAERGAKTSHSTFDALSSANVTVLETISRDTPRHSSELM |
| | | KVALSLYKTNLKNMWAKNKLFNKALNKLLEILLRIHLAPKRESHYQKLK |
| | | EKRAKKQQAKNTFDAVAASTTGSDVDIDDMPIEELLPHYEVLDKEDEEG |
| | | EQGEQAEEERGKQTGSRDLPRRRILVLKAVLRKLLVKQAGKAISHAQLK |
| | | KERRDITDDEARVCLLLSEILLPYIPAKGHYSYIGFQLPFVLLANDIFRAVG |
| | | YGKFAVDLCPTSSCGSLHAMRLDAPSVYTLCTKGDDPVDVFDFEGFHIPS |
| | | VAEARAHKHAMFNTFFDIRAIFKACDTFGLTFANNLTILPGLSSVRILGTK |
| | | KNSSKPAYSMKKPLDIDWKFVNDAQSKTDEELRKDIDTLEADMITLSEEL |
| | | KSQSKDHNHEELSSKIKQLNRQRGAERTLHDRDPCSEIADLKTQRYQSFL |
| | | AVKRTKQQLSRLRSRAFLTRKAIEYRNKEIPAAAVTDPPPSDQELNAMVF |
| | | SGTDNGLTVMTETVAMDINKYKYHLSLHNRFSPLANFDINEGDGSEGAN |
| | | AATSDIDKDGDQASSGDSSGVDALRGDAVTVDTSAVPQTHDDDAASDG |
| | | ASNHSATFQPLPTSYKMRAEDVDFGSGQFTKRQIILKAKRTTEQGKQVQ |
| | | QMEEQLKTSNVFRAKHVEDVMSNLAAYRNNGPALRSFYHSKKMTILSN |
| | | KVEHTDVRFRSRFASRERSFCAGGRPQTHLTMFIGDRGLCVGSRLKGHM |
| | | KYGGKWKPRLHSSVAKVHTTNEHKTSQTCMYCFGPTSHLVQTIKAKDG |
| | | KTRTKLTNGAFLCLDPECVSVVNHRAIQGRDRTSAMAIAVSGLSSLLFNQ |
| | | PLPVFNPKPSQSNTGIISKTTSFCNRNETRDGSGAASSLP |
| 40 | MCi-7 TnpB/ | MAKKRRYQETQFTSNPNKPPEFRRVGQRSAPVTATTTPSAKDISIFQAWK |
| | Fanzor | AFEDGLREVLIQRIKGARLDKKPLNKLKKELQNETRILRYTYSNADRIHK |
| | | LKANTTRAKVQNSKPLDRQKKYIQENFDTIAQSSESLPTPHRTPGAPEGR |
| | | QQTTATTPKQEFKAVTTTFSSIIRRQDISDERWRHFQQRLSEATEDVSNHII |
| | | DFGVQVLRLITMFKTHTFQLQGDDFQFVPSQASFTMQDILPPRYHNEETP |
| | | HPPLPPPLAQQVLNNKRFQDLARGFLSEEHLSRINTTYLGIGISDNTLAKY |
| | | PLDAAITARLPRPQDVPPIRFSSARAVARNLYSTNFDNLWSLKGRFTKVF |
| | | NKLLTILVKIHLAPDREAKYQDYLKKQKQGQEKKTTSAQEANTGNFSFA |
| | | GKKRNQVRCIFGQERKNQQRYNQKAKTMGGKWIKRARRSAECLQTYAF |
| | | TRYQEVQDSQQAKSTDLTAREIRQLVATIRNVIFQHSGQAQITAKQFRDV |
| | | EASVNGHQLDVCLNIINRLLPYIPATKRTWFSKMHQIPFILLANEVFRAAG |
| | | YDRFAVKLANIPSTSTLAALHLDVTSIYSIFSVDEVKGRTLDLFSYTGEAIT |
| | | SRSTALSNADAVFHSIFDLAKINSMMDLYKLQFYHRIVLLPGFKTVRILGT |
| | | KRTAATVHSARATSIAPQDLPPKPVEKKNLTVAEKKDIENKITTQQAEVN |
| | | ALARQMKKNLQERGEFLRQYYQDIKSYKSLFPTKTNPPTSQGDTAEERIL |
| | | QGTRDEYHQYINQLKQTRDDFNRTIMTTRKELAEARYQLYLRRRTTSTT |
| | | PTSESVPSTTMIKQITSSNYECYEKAENVRLPENLAKAPNMCFAGTDNGI |
| | | VTTTETSRFNMQRFQFHLKLANKFDVLQDLEDNPELIQVPKTFKTYAKDI |
| | | DFKTGAKKQRQRLLRDKSSTKGQAVQASENICSQNSLHDAQNINDLNER |
| | | YQAQLEHREAIRQFYYTQTRAKQKRKYEYIKRKQIDLLCSQERQYVNNY |
| | | NSKSQTVLFIGNRGTGVGSRIKGHLRYGGHWKSKTHARYTSVCVTNEYH |
| | | SSQTCPYCFQKTTHPVRLVNGKLITINGTSVCCNPNCLLVRSGRSHQPRDS |
| | | LSALVIGLSGLCTVLSGTTFPALDPKRISHFNTDFQQFAGSFLTRNLPRPA |
| | | V |
| 41 | MCi-8 TnpB/ | MGALYATFKAKLKEAIRQCPDRMNYPELYITLFERTKLPRYTVPDAARR |
| | Fanzor | LDIDLRLVESFLRQQVQTTGKQTIAYWKEYVEQHFDECINEVLYVDPQET |
| | | EANSSDVDDQDDDDGYDETEEFRTCTTSFQTIIKKDLEEEVRHRIEETILN |
| | | CIRDTTNYTSDFSIQVLKMALVFNTHTFTLRNNTVILEEAQGANLGHVIPI |
| | | EYLDDAMRVPRPFDAACFRNQAFKKDFDLLFQTGHFNIINSSLYGTKGPT |
| | | DANLNKHSVHRLISDAIGLAPTEAYHHLPSHVTKMARLQYTTNVKNMW |
| | | SDKTIINKLLKRLIKCLLCIHLRPTDEKQLQERRLQPRQAAPRSSNSNQGM |
| | | SFSHKTRNGRRRVINNEKKNLERCKRLNKVDKAARCEERLTMYADVME |
| | | RERRQKALRRQASSSLEEGVSEDEEDALVADEPESEKTNDTSKRRITQML |
| | | NQIRSNLFQNTPLSRSTVGSKIENLSDSEFDAMSIIAGFIKPYIPAQVDYAK |
| | | LQYQLPFLLMANEILRYMGRSSQCLQILPLVKPTTLHTFLIDAKTLYALFC |
| | | ASTLQQPMILFNFQNEPIQEGMVDSYKDAVLSAFFDIAHLKRLCERHGNQ |
| | | FARRLHIFPALTTVRIYDTVPRLVQHQQPITATDGDPIDEDHHPDLQQQN |
| | | DAVVRFQHELKDATARLETMLLMSPSVRQLKNDFRGGQASFKTVEDAK |
| | | TTKRRLMLDVRDAKTNLQIARREVQRLKSMHKFPPTLPPNYQRYMIRQQ |
| | | PCLKKAEECFIDEDFDLQGAIFAGTDNGIVKTTETVYFDIERFLYHLRLFL |
| | | HFHPSDQNGQPVGAPAMFMDLPATLTIGSKEIKQGSGMFDYMRSMNKK |
| | | KTPGEQVLAIESTLSANSLFNCSTPNELEETAHLHQRHQQAIRSFYYSNIQ |
| | | CKERRQFEWRKERYYDRLASNERNSSGVKHPIMFIGDRGHGYGSCIKRH |
| | | LRFGGHWREERHSRYTPTIITNEYNSSQTCLYCFNKLSHPTFAHNNAIKTT |
| | | KGTFVCLNEDCPNAFVSVCRDQVSALAIGLAGLSQLLFGAAFPCFDEDTN |
| | | PMNKTRFYEMATLFLREHA |
| 42 | MCi-9 TnpB/ | MSSKRQQKRKQRESFDPNVTKINPAAAFASTKRRGSNVEITTSRKKQKIT |
| | Fanzor | AATMDNALKSQFHDYIFDQIAASTNTTDTFSSLISLKKMPRVVIQAEDSIK |
| | | ADRGTISNYLRGLPQSNISLFVNYAKKNFDDIVEEVEATPSASSAGPSTAP |
| | | PEPRPSTSTSTSTSTSTSTSTSTSKLIFTEDEDDGDLRGCTASTLSILRQDML |
| | | PEAQAHINHTLEQTLARVSDYIVDFSAQVRKLLLLFRDHTFELDQNSVTF |
| | | PLVAPLSSNALLPTDFLPNDVQLPPHWPSKLLDSKKYEHAFGQLFTLQHF |
| | | SRIHATYYGQIGTKTASPYHDAMVTALPKTDGNEAASRQLSPHAMRKAY |
| | | DKFSTNFANMWSGGNRFAGLLDRLLLHLLNAHLVHRAQRERKDKDTAQ |
| | | TPSLQQQTDTQYTFGSKSKGTVKKIIEKEEKNVQRHLNKGNHDKAEECR |
| | | RRISNYKATQIREIKEGIAGVLNITLQIYKSTSRKGKQKVPSTEIVTLDDAE |
| | | EPPADESVTKTRIQDLKTLVKKLLQDDKKVSSKSLQSVFPGIKANEMEAV |
| | | TKIVNTLRPYIPRKGKSGFIYKLPLILLANCLLEAIGKRSATVSISPVSSCVK |
| | | LQALFIDTETIFSLFVASKGATKAERVMQITTFDDQPIKSASQALDNKSAV |
| | | FSAFFDITAIHKICQSYHLSFANIIYLLPGCKTVRIMGTRSRLVHDLPPPSLP |
| | | PEASSTTAAAERRAAVLKAEKEACQRELGEQKQALADFHRQTSIRKLKN |
| | | EFNNTKEARQAIQTAKEKSHKLEFQVAESRRAIQMKKQEIQALTWVPPK |
| | | KQPTLVDTDAPCYKRAEDSLLALDDIRNATFSGTDNGLITVTETVAFSLL |
| | | DFKYHINLHNFTRKLDSAKGTLNSLQLHTAIHLLTSMAADVFKPPPPSFK |
| | | VNPRNCSHWRGLSEFQQFIKRHKSKQLQQVEKRMLEHPLNVSSPVELGD |
| | | NYTVHHSAAPFLRKFYYSTPIVKRRRRLEFRSKKFKQKLCHRERAFCNKR |
| | | RPPIMFIGDRGFGIGSTIKKHQRVGGTWKGKLHGSSTKVFVTNEFNSSQT |
| | | CLYCFHKLSHTYKKKDGKKTINRGSFICHNPECVGRFRVLPRDRVSAMAI |
| | | GLASLSQVLFGVTIPEFDPHPTATKSKEFNKKAEEFCRTFIV |
| 43 | MCi-10 TnpB/ | MRSRKKQKAPSINASPVNPLPTWVNSSKSAKRKADNISGDTAEASVNHT |
| | Fanzor | ATKIEQALETEMRSRLTKKIKACLTKGDKLADLKKECLAMARIERGTVS |
| | | NAEISAGFQLNTITNYLKNHSKANIDFWRDFVKSHFDTIAKEAQASIESSA |
| | | ERAAASSSTSAQPKPSTSASSQASASSTTSTQATISAPKAIVHQDTIRTCSA |
| | | NFNSIIRADLPLDIRDMLNATLSRTLEHASEYATSLGLQVLKLLLIFNNSTF |
| | | NIDKNDSITLVNAQGFHINDLVPEQFHVEEDKQVAPPLPKQCADSKAFSK |
| | | EAKKLLSFQHMGLIHSTYFGARGIAAQSLKANPIHNALITILPRENVQPDL |
| | | ENFVMKTVVQTYSTNFDNMWNNNKVFTKLFNKLLLVLLRHYLAPNREK |
| | | KRRKYIEEMKEKRTKSEDGVVMRLELMADESSKVEEEVLQAEDDECKP |
| | | VDDSDKSRRHINVLTSMLRNVIYQNYGNTVTEKIIKSTCKDIAQDEIDAFL |
| | | KILDRIMPYVPSKQNWFSTAHKIPFVLMANQVLSSLQYSKFAVQLCPLTP |
| | | VGDLNALKMDSRSLFAILCSTHSKNRRALEVYDFDGRPIPSGDAALEQKD |
| | | AMFGSFFDLNKVKQICQSYGLRFHQHLVFCPGLKVVRITGAMIKHPVPC |
| | | NCDGSLKKLLKADLAATDRERDQLLKDRREFRKLNQIKSYKSQWNKGD |
| | | KDGNKAGSKTSGETISNNELYWLIEKEKEKRDKINMALGNNKEKLSSLR |
| | | RELYLVGRYLREAKATKASKKPPAPPPELKTCIKKGDSCFLKAESTTLDE |
| | | SLLDQDCCYGGTDNGVVTFTETSKFDLAQFQRHLKLYNKFQCLETLGIVI |
| | | EHSCLSFNRTNMPLEGAKSKHVLLPRTFKITSKHIDRGSGQQKRRLQLEK |
| | | KKKSTDLGRQIATFEATKSQNPIFGATTPAEVDQLRSVHESHKQQHRDFY |
| | | YSNSRCRGTQRYEYQRRRFIDRLCADERRHISNNSPVLFVGDRGYGINSRI |
| | | KGHMRCGGAWKARIHGRYCPVLITNEHNTSQTCVFCFHKTSHPLKLVNK |
| | | KNKQYLRCVNGTTVCTNPQCLLRAIGETHKARDSLSALAIGLAGLSTATI |
| | | GQPIPQFDPNISASKTEAFKAAASDFLMRNSAGHAPRV |
| 44 | AGo-1 TnpB/ | MPIFAAKKRQATLKKKLVLQQGDELKKESLNIPSWAPFKLIDHGQAAND |
| | Fanzor | QVLIPQKPEVPGDALKDHWTPKTVRIYPTSKQKEILHLWLTATRHMYNL |
| | | AVAERIRLGKEAKENGTKVKMKLKHLREVMPLKKNFDYERDPKLTYLK |
| | | NVPFLIKDNGLRDFDKDYRTNLAKSKNTLRPFKIHFKKIKDKQSMTVEKK |
| | | VWGLSRGIYHDVFRKGAFRSTEPLPEQLPHDSRLIREHGKWFLVIPYETS |
| | | GLMPKKEAINAVAVDPGVRTFLTTYDSSQRVLEIGEDGYLKLQHLDRRL |
| | | RSLQSEIHKLRSKGIVLSKEDRKRKIRNRRTVISRLYQRKKNLANDVHRK |
| | | ASHLLASKYNYILIPVLNFHNFKKTNRRVKANLANMKHCLFVQRLKNKA |
| | | SHFENTTVIEVDEEFTTKTCSLCGVRSNIGASKVFKCDNCDATFDRDHNA |
| | | AKNILLKYLGEID |
| 45 | ECy-1 TnpB/ | MPIFPAKRQATLKNKLVLQQGNDLKKQLDIPSWTSFKMIDHGMDANDQ |
| | Fanzor | VLIRTQKIEDPGVSKEFWKPKTIRIYPTREQKQTLQLWLTATRHMYNLAV |
| | | AERIRMGKEAKAKGTKVKMKLKHLREVTPLKKNYNYDEDPKITYLKQV |
| | | PFLIKDNGLRDFDKDYRTNLAKSKKSLKPFKLHFKRIRDKQSMAVEKKV |
| | | WGLSRGIFYDLFNAKTLRSTEPLPEKLPHDSRLIREYRKWFLVIPFESKVQI |
| | | PKPDQSVNAVAIDPGVRTFLTTYDSSQKVLEIGEDGYLKLQHLDRRLRSL |
| | | QSDIHKLRSKCALPKQYRKLKIRNRRKVIRRLYQKKKNLANDVHRKASS |
| | | LLASKYNYILIPVLNFHNFKKANRRVKANLANMKHCLFVQRLKNKARH |
| | | YENTTVIEVDEEFTTKTCSSCGTRSNIGASKVFKCGNCESIFDRDHNAAK |
| | | NILLKYLGEIA |
| 46 | SCe-1 | MNIKTLRQMLINKDINILEPHEQWLKEYNYDLKSEALRDLVKNYSSNMA |
| | (EGA80285.1) | RYKSTGTPFKLKFKSEKAKTQSLSVLKKYWNNKKSFYSDVFSSTKMRSA |
| | TnpB/Fanzor | EKLPEQLLMDSRLQRTRHNHFYLIVPMVGEEIARDEGREKFIFIDPGVRTF |
| | | LTGYDSGQNVVEIGKNAVVRTEKLKHRRRKLQSKLAKLKTHKKRQNHR |
| | | KALHRLDEKISRILSDMHKKSALCLCKSYETVFIPKLNFHQCTKLNRKSRS |
| | | SMATMAHCSFNDRVVMKAEQLRGTSVIVVEEDYTSKTCSKCGNIKKDL |
| | | GSNKIYSCVSCFSVFDRDFNAAKNIMLKYCCEHLMTSDASSVPLPLVERV |
| | | RGSP |
| 47 | TDe-1 | MTTCTQYQAKKRKRSNSKTTKPYWNEDAAHISQQWGLNTILPTAAKNL |
| | (CCE93668.1) | SQKITSDSWFSVVKHPQVPVINDSLNLPLSSGVKEENTRARKIKVYPTNSQ |
| | TnpB/Fanzor | RETLKKWFRCQRYIYNRALKLHRDGMAMTIKGLRQTLLNRDTIILEPQE |
| | | QWLNDYSYDLKDEALCDLVKNYSYNMAKYKQTGMPLKLRFKSKKTPIQ |
| | | TLSVLEKHWNKRTKSFWSEVFSLSNFRGTEPLPEELPGDSRVQRTRNNKF |
| | | YLVVPLAGEEIARKSPAEKFIFIDPRVRTFLTGYDSKQTVVEMGKDAVVR |
| | | IEKLKRRRRQFQSKLAKLRKHKKRQNHRKALHRLDEKIQHIVRDMHKKT |
| | | ASYLCKNYENIFLPKLNYHQCTRLTRKTRSSMATIAHCCFHDRLTMKAE |
| | | QFHSTSVHEVEEDYTSKTCSSCGNLKDDLGSNKIYSCSSCSSVFDRDFNA |
| | | AKNIMLKYICEHLMASGGSSITSASSGGNGGFAMMGPGPFVR |
| 48 | DFa-1 TnpB/ | MNGPLPSATETRTTQYPLDRLMGKKWNNEQNLFVTHLGQYARYYHSVK |
| | Fanzor | RLVGQFFLIFVYDENNMFSLSRQELEKVIPKYYDMSVLSKNKTNELYEK |
| | | MENRLTLPIRWDSLIVEAIISKVMDIDTRKSSNSPRDYVDEMVAKYQILR |
| | | YALLDPDLNPIGCCHSYKTAFANSICNSYSAEVAETCKRMVESALDRQD |
| | | DRNIFHHESKLVSDLLEDILAKIDDGDIGEGYQALKLIFSSQPLKGMSLILT |
| | | PSQVFFKYLLAFNSTFIPFSIELFVSIIVNSGIDMSLGLTDKHKILRYISKILP |
| | | NLEKEIGHPLESGYPLDRFKFFDKEIEILKASHLASFPSSSDSLASTKKSFLF |
| | | RYYTIGFVGKSVLCDYLQSPYVKDNPFDFNGSTKTIHPNFNDYERDNIGY |
| | | IYNQKMSVLINQRAMMVTTNVPSHIWFQTKKEMEKNHSYSYNHPHYAC |
| | | FVAKQVGLKPNIPKVKDRDIICANCLSSHSLKDLPMVCEQCDATIPILSEH |
| | | EKRLDLCGDLMDLYSVDIQLKRNAFEKMPPFDSKDDLFPPPVPLIAEINH |
| | | RLVKFDQPESPHVEIFLHEALNTTSIALNSNRSGIAPSMRNLFNLIKTTSDP |
| | | SMQFRIRDAYPFVHDSSTGGFENEIKHEIMVKELMNHFSMTTSKLIIVCGQ |
| | | FPCKAAKEAFNANPLDYEMVDYSQESYHTILDKAKGTTEDKEVDCVYK |
| | | DEEKHIRAKRDFYHSGMCRRRRSKWKVMRENNRGNYLYIGIDPGRSKC |
| | | TIVDSMNRARVVSVASYYNLMGTDRLLARQQNYKKNPVLSKFESGLPPY |
| | | NTFDTLFNVESVLRRIEYERITHRDQCRLYSHVVTSYTFQTMDKNRNIDK |
| | | KRAQYAYAKSFVPPTTIPKNRIVIYIGGSDFFRGSRGGGRTGCTSKTLLRL |
| | | FLNMGYKARLVNEFNTSKIHNACASKIHSTNNNKFLEKAKHQKVKNVLP |
| | | ESFEKTHVVGAPISSTFSDNGVRWQGKDSKLCDGCKLTDYNCQCSKAQL |
| | | ADKENDSTLKVSQLADDMNETLKVFISLENTSPSPTMLLESLFSLSKKTV |
| | | KELIEFCNLNSIKYNQQEEPTKGKNKRGSHKDSKGGATNSKKDQLINAII |
| | | KFNFEKQKRDASKNKKKRSKSSSTTNDRCKDQDQSNSQKDGNKSTSNSV |
| | | STHDYGFGKSLCDSQIKDLWKSMNPNATEMPFKTKWQWLYCPVCDHFF |
| | | NRDTSAAKNILLLGILHQTADYRPWIYRPLPADIVHIGRVDTT |
| 49 | DFa-2 TnpB/ | MIRYFALGGDAIFSAMGKHPVPRIDTVEDLSNDSIYSLCFFMKHVAPFLE |
| | Fanzor | KEMQKRPKMLQMLQKLRQFRCNNSKNWVNVIVINIKNNIMVSTYNHLK |
| | | QLLNTYKDRKIKDLGKNQEEIKKVKKLVRDTIYNFSNTRLKRAEKDQQP |
| | | VQEEGEEDEEGEEDEEGEEEEDEEKKADRVVTYDLSDGVYKTIEILGGY |
| | | NQQVIDEYNNLSDDKKKKKEAKYDNRVSRRKNIIKKKKEDAHKRREAII |
| | | KERREASIKLQKSPNTSLTVDDLTNSPSSSPNTSSPRNPKGKFNQRMKHDS |
| | | IRIRELDELKKITIEAEKRNADLQQISDTRTPTVFIPGLINISSIIPSLGFDIQF |
| | | MFTREHAKNLLKCLGLKKANVEKDHEGNPSYAIATLFPGLYKYDPRLFT |
| | | NTKRVYLPATPPTDVEKQKDEERRKTNNDNRFASALKKYKGNEDVARV |
| | | AATLQTGEKAKNAIEQHNKLHGFKKGSHDKNRALLAKGTITFSKEEMFQ |
| | | EIVNKGVKYENTSTTTTPSTSSSSSSLPYVTVTFDKKVSVTTNGASATVTF |
| | | RTIKYKPGNKPNKLYNKQVAMEKEFAGKSLKDVIAIGIDPNTCSITVVNN |
| | | HDDKVLDMRMDTLMVKRNTKFLLEKISCHMGKAIKSFESCLPSLKFNVL |
| | | NPQTFQSRLDYYEKYCDYFDIYFEAINDPIVLSWKRNFAFKKQKLNKEFY |
| | | QQFDNYILDLFKDHFPTKYSSMNDSERNQYIQDNTIIFFGNAKFGHNTRG |
| | | KVTTFAAKTVANKLKSKQYSVVYINEYNTSRQHFDCFFHQEEENLIHHD |
| | | LNEPHEDPKDGFHLEKCHLKLGKEKDGGQPTNVQQCSPISEACQSAAKG |
| | | NSFLRCSDHRHRLALVKQDTHSKEYKESLGFLKSIDDLDKKLQSHNLLN |
| | | VAGQCRTRKVTPSDVAGSETESRQDSQKVASTQSSSSSTKKFDYRHGQN |
| | | KYGPEIRKASKSLHNKHRMAVSWKRVLCTGCGKSFNRDVNAARNMLLL |
| | | GIFKVWKNGHRPSRYGKTN |
| 50 | Sola2 DFa-2 | MSSAAQTITASPAAVATTTTITTPPTGAVFQPFIGNRVLRSFSISKHGRGIK |
| | TnpB/Fanzor | MLLDPKGEKIYNDGTVFMATRDYSCLNIIRKMHKEAKDGKKNHFTKTQI |
| | | LSSVSPFHTASQLRDFGFKFANGAFTNANKHAKQYGPGRNIRAEEYTQFC |
| | | QALGRSDINKTDANKKKIMEDIKEFYAQTTITEPSSKCSTKNQKDANNKT |
| | | VRVSTPIRKLLCPSIAQAYAYYVEFIAKKYKDCSDNDEKTPIHRSTFYKFV |
| | | PSDVKIFNNRTDTCPYCHLYDVLKAKAEKTTLTEKEDTDFKTCKIHKEFV |
| | | EKRKKDFEKKKESLTKDQVIVLMDFKENIALGMGPEELSQNFYGKPQRSI |
| | | LGMVIYHRDPATNEVVKEHFDVFSDVLNHCSSYVIHSIKLFFSTSELFKAR |
| | | HFSQIFFWSDGAKHFKSAEILSFCIDLKKSPSQSLNRVEWQFSAQYHGKS |
| | | DVDRHFADLSNITAQQSKCYDIYTGKDFIKGGDRCYSFSDYNEFVFTNKS |
| | | MYMGTHEVNYELDPIRFTYKFLSQKVKVGQEIDIKRGFKEVGVEEAQVV |
| | | KLKQRMVNATKIKLNIQSSSSSSSTSSTEQYDEKTRISELKSLNVKQLKVI |
| | | NKEWTFRSKALKGEIIDTIIKYEKEAIAEKMALARAEKINQEKRDNLIYFQ |
| | | QQHEEKKDNKRKNTEEQSTNNNIIIQEEEEEEEEERVFKKRKTHNNAIVD |
| | | DDEEEKEEEAAEANSIKQAIEEKRRKFNAMFVDQEEAIQTRHHPKKDNE |
| | | QDVIMGDIGGDDDDDDMEDIPCTEFKDVCGTTPILESIQEETEERKKLKLE |
| | | KKILSIVKTFEQQIREGKNNITIDKPASKPQEQGQWNLKIFSMDALFSIENF |
| | | KNTIDKIIQLLANYFKGYSLDQGFNISLVPIL |
| 51 | DFa-3 TnpB/ | MKDIDKCLSGPLSIGKALIDAVLCKVTQVEERSSSSLPKKYRLTENQKIRN |
| | Fanzor | ALKDSDLAPIGTCHSYLTEFSNSIETQFLAETVETFKRHLTSLKDYIHDRRI |
| | | YLLLSYEVCELLQGILDKLNGGGNDIGEAIEAIKLICDSQQLKDHNLRCIA |
| | | KGKKNLEEREKEQEREKKQARGKKSKKNYDLENEMADFSKINIDGKIITI |
| | | KELAETALRLDAGIIPTNDGLPQVNERMLSETSKRLKELIDSSVSPKSSWS |
| | | SLDKDQLNRLKLFNLENVIWPKPLPQSGLFQYYTSGHVDKSLLVDYLVSP |
| | | LPKPDPVFDKKNPPTVHINFNDYVKDKIGYIYLQNVSIRINQRVMMITKN |
| | | TPGPVSFREKVETDKAKMSPNHPHFPCYIAQQVGLKPSVTKEIRKITCAH |
| | | CLVSHTLNSECLPIQCPTPLCNIQIPILSEHEKRKETCGNFSNLYNVDPSLK |
| | | KAASEKLVHSSDSCPPVKPAISQVDRILREYISTDSPHVEIFLHEPLNTTSIA |
| | | LNSDRSGIAPTMRNLANVINATSDPSMSFRIRDAFTSVRDSSAGGFEDEPK |
| | | EKEMAEELKKQFESTTSKVIIICGHVACKAAKAAYEVNKKDYEIVDYSQ |
| | | NSYDNIYDDGVPLYENRRAKRNFYHPGMCKKRRYQWRSIRQAISSKYLF |
| | | VGIDPGRSKNTIVDSLNRARVVSVATLYNLMKTSRLCAMIEKYKMPLKS |
| | | FERGLPEYNTFETLFSLESVMKRIEYERVTCQHQCRLYVQVVTSYTFQTM |
| | | DKNKHIDNQRARYAYAKRFVPPTTIPKNKTVICIGGSDFYRSSRGGGPTG |
| | | LGPKSFYRLWKNMGYKALMVNEFNTSKIHNACAASIHLTNNEMFKDKI |
| | | KHQKIENKQLPESLEKTLVVATPPISDNGVHWECNFKKDEKCGKCACKK |
| | | YDCQCANGDLAKDINSSLETFITLEKGSPSHTLPLESLYSLNQKTKKELID |
| | | KIINLIIKFNFEKQKKTNSNNIQENIGHSQEDGNKFTNNSAPTHDYEFGKSL |
| | | CYTQIKALWKSKYPGLDMPFQTQWKWLFCPVCNRFFNRDTSAAKNILLL |
| | | GILHQTKAGYRPWIYRSLPADVLHLGR |
| 52 | DFa-4 TnpB/ | MINTINHAVVMVDQLWKSELAIHQLPTKDLDDVDEIDHQISKLTPDQRK |
| | Fanzor | AREDIMKKYGDLHIKLEAKQKVDARVANIADEVASLIKFGDSFQEFCKR |
| | | KESDRLEAERLVQEKETVDEKKKREEKEEEAAKKAKEAASRLQKKRKR |
| | | NKSKKLASEKRERIRAGIEKNINDIIDFINKKPKPSAIKRSKNQSKTKASRK |
| | | MIQAMNNSFIHQRISNLQKENNKAYKEQMREYKKAKEIERIISLHPSERT |
| | | DPEEEEKKKKIPGMSNLSPIVPIAKLDAQKPFTSNLLEALLIYYHKDLGIK |
| | | KDFNPSEALAPSLFDGMPTVTCQSVPPPSLVYSPDSMVTLDHKFASISVPT |
| | | LTPPSLPPVPSLPPEPSLPPVPSPPPVPSPPSKKKVSKPSAGMKKKGGPPPV |
| | | PSPPSTQKVSKTNKFMKFAGKKKLKVPAWSSFHMDTPSARRPHTLVSVS |
| | | LAWNSRHPNNPIDTRKNRIYIINQLNERGVVPPPQKPIIAPADRFKDIEIGKI |
| | | RVFFDKQAFFTTDGASITFTYNTIKTPNDIIDYGKKILIDEKDRVPFSPKTIY |
| | | ENDIINKMRDENMEDVMAFGVDPGRRGNYWTCNQLPYTFDQRGDVRSS |
| | | KLGSKFLNEREKALFGDKLYHFQSDLPSLKHDVFDPQTFTDRLEYYKNPL |
| | | YLEYIETKTDRLQRLNWYKNHSFRKKRLNQSYFKEAEVYMEECWKAKY |
| | | PEMIQNRSAEGKKKFIKENIFGAAKFSHNSRGSPTTFKASTVAGHYKSVD |
| | | YNIIMINEFNTSRQHFDCWARSEKNHLDNFNKHVPHVKGEPFQFKMGDL |
| | | RKRELNMDYLDQQIMCNDKDCQSAHDNGHTHCCQLKKGSLFSLDHLQS |
| | | QAGLDEFLQSNGLLDENNKGRTIDPSYRSANKKRKKKDVKNNNDTQPL |
| | | NNDGMEIHGTHSQTVQCTSTSPKSDDYLKDQVLYNRDISKATIVDMPGC |
| | | HVYVPWKTAYCTCCKRSFNRDKNASLNILLLGILLIWKDLYRPTRYKGIT |
| | | NIDEATIVICTQLSKKNKEDFIL |
| 53 | PPa-1 TnpB/ | MVNQIVRDHMKNQSNLNLLNIERISKHIMELNGFGPIVTSIKKYLLAKPEE |
| | Fanzor | TVDSALREPSILIWLRQHDATITKESFDRFKSFKNDFETYLVPVTKSSETN |
| | | CTSALEFIQSILNHPTIIFNWNQHTINQFQNIEKEFGKYHNEDNYKTESQA |
| | | WINSIKQPTSPIPLKNNNSRWIKANDNYNKLIIINEFIPKFKLAPRNFILSTK |
| | | FLQQFLQQFYNGSDFNREGSYLDHLSLLINIPEQFKQNFYTFKHPLTVCQF |
| | | NLDRVSHCGHVPPVDSVDHTNAHTICMFAASNNLTLTVPKQKKVGAML |
| | | CPEHSTFSKKCKLSKCGSRCISKFKESEWRIQSIRCGSLDSEPHIEKKNQWI |
| | | RDLSKPNSIIEVTSQKQTSINWTSTNASTASVKPNQEHFNSPFFVGCDINY |
| | | QGMESIPPNCDIGVIFSDPTSKWHVGLTADDTDFNPSFKKLGKIINTVFKD |
| | | KKVLPKLFITNSYSSTNYSPFGIPEQSLPHILAWLDQIIEGNHKVIILADWE |
| | | SQSFILHLFSGKPIQNSLYNISNQNSGNIESYILYHSGRPTTLFIFANHPTPG |
| | | VMKPELELASRLLAKYDGDSKHFILDPAKEKKLNFKTKPSDATNTVSYD |
| | | YDGPTVSLNGITFSITYKKPKTREDDESIHPSKQPYHSSLNVDLKEFQYIQ |
| | | DMVKEYKSNKYIfgIDCGRSSHVSIDEHGKTHSFSVHEHYLNLRSRFNSEA |
| | | QKKMLGPAMLKFESLIPPSKSKSLISNRISYLRLHPTTTKNYVNIRLDEYN |
| | | LIMKKNNYYNKRKSYNSMVDKLVPIKNSIVFIGSAKFSHNSRGQSSFISSA |
| | | TLYKWVKKKKHKALWINECYTSKVIAECHKQLSTLHQTVTGYIDGTNK |
| | | VNSSTNKSNERFDYMKHHSLYSNQMSGNNVNVDGLAKDHPILKVFQEK |
| | | KWKRPLCRCCGHFYHRDKNAARNILLLGLLSVRYQFRPVVFQYIKFINK |
| 54 | Sola2_PPa-1 | MYLKFNNKEKIKQSIDLIINRNIVKEVHQTNTYYHVAMLFCVLLFLKKKG |
| | TnpB/Fanzor | KFKVSDSMVVLLXQKNSERDIRRYMKRYLEQPHMLLRCHMLFHHFRVA |
| | | NIQIEMDMKKQGKCFVMLAILYIIDTNIPNLDCYQYSQSRLLSSLVPFYTS |
| | | KEIREFGITIDNNAWNRAIQHVHKYGAGRLINAEEISNWRSKFTHNGQMT |
| | | RRQRTTMLRTAVEGYFNMKDITEESSKNMRLYSKELDADGKRAFELVPI |
| | | KNLALGNINTCFTDYLKKSEVDQVKYPLMGRSAFYSFVPEYVREMKNRT |
| | | DMCPMCVQYQALESKRIKFGQLSDQDHKLFEILTIHKMFVQQRKNDYLT |
| | | CVIIILLIHLVNDLLMKFFFFFIXKNELKAGECIILCDFKENISLGKSKVQVS |
| | | HEFYQQPARTFFGMVLFYKQGDKLKIHYYDCLSQVLNHCSTFAISCLDK |
| | | LINDPLFTSLNINKITFWCDNARHFKSNEILSYMHTLSKLQVADPNTPNKT |
| | | KRKYLVELNTFAPYHGKSECDQHFSMVSRAIADKSSFKDSIYTDQDVINA |
| | | IKSTTDNNNYQRTYKLTSESEKEAVTIKVTVEIHNPKELKQKEIKIFPGFTE |
| | | YHRFQFLDNRSVSIFFYKKKMRANKHYIKFMGGNEVDKMTIYKYSAMK |
| | | SQVGKETTISRGYESTTKEEAQSWKPARDKAHTTYLNTMIEMNEIAKNV |
| | | DESNKEKAKEMSDIINTYANVTSKLAPQPTPSRTQQIVSSPQSPSQPTLSNS |
| | | QAASTSSQSSPKPATTTTPDQTLSGTNKRRRGYRGTKKTKQTNSQNNTQ |
| | | NNTQDNIQNSTQNNKQINTQNNTQNNTHNTHNNTHNNTQNTQSKPLFDI |
| | | ESIDFGDDDHDIGSSLIYSGKRKEREDDCDNNSQHKKINNQINNQINNNN |
| | | NNNNNNSTKKRIREEDNNQESMDNQKHHKSTHVNSQTPIQSPSQTPIQTP |
| | | SQTPIQTPIPTQSQTPIQTPSQSPIQXPTQTPLHTPIQHNTTVQSTEQSKPAK |
| | | EIVVIFEIDNNYNSQIQDYRQQQQINYLQQLSQTLQQQSPPQSEAAASNDV |
| | | EMTSHEDPIMEQVEETDDAMEIDRFCIPYEKIKDQLNTAEEDENMNNAG |
| | | DFLSNWPSLRELIIRIPCLTAIADYGQLFSTLLIKNPLSNLVIRPIQNMIGNV |
| | | LKSFL |
| 55 | PPa-2 TnpB/ | MVNPTDLIRKDIIMDSGRQFFTNTLNNLENIYKRFSNNSSSYLKIKDWEEE |
| | Fanzor | NNNNIDTLSKYNISISEYQQHNRIKSTIEKLKKTKSTDIRITEHLSTISDLYN |
| | | DSETIITQNSIPVHETLETTPDPHQYLWMVGLPLVIIHPLTEDRHLEDFFTL |
| | | ENYIKYLSSNVISEDYFYTIQKHVFTFHLFLKIYNILSKSKVDKAKKSLNS |
| | | DAFILSLFDISKKPLDVLQSINAPDSKFFFKPSVKFQRFPDPFVAASLQPHFI |
| | | LIDDLLSKHLNKLTGVYSTQDRYVDRLRKIFPDLDYRNLFHNHSLKPSVP |
| | | TPDISIKEAVSIETLRSIISKEELELKKPTADEINQFKKQRLCPRHLKPMEEC |
| | | SQTCKDEFQGYQWAVDLFSEGFFSKSKDLAKLWPKYRSTYIDDIKIQFKE |
| | | PQVHDIKFSTKQPTPEPTTSNLENQDIPTQFANTPAVVSCDSNLLPIKVTD |
| | | HKDIMVVMSDPISSMHLAFNREDNDLCKTFANLFKAFGIKKPREIFKLND |
| | | IKARISKRPQRKKIYSPSWLISKEGFTPVSIYEKSAPVIRTDGISLSITRGVA |
| | | RRFEPKLKEQHLPYQKAGTAKERRSIETLKMAVATDGTNPICYSIDPGRS |
| | | SHVSYSTDSKERRASVKQYYDWKSNRYSKKVKKLEEKFKLFQSKMPPH |
| | | SNAYGRADYFIKNWEVMKLYIDFRLNNIHRQRRRLEFKLNQQSKSKLVK |
| | | QLVPTTASEMKQGVIFLGASSFNHASKGHRTFISSKGLKKIFEAHGYNVV |
| | | SVNEFCTSRVHHGSYVEQEMKLLSAIKRVDQHVSGPKAQWPPESKPKVN |
| | | SSRGSVYEKVRNDYENLSKFESDLNSGGSMQEQQSTYDYYGHHSIYSTE |
| | | LSKKTTKSDQGHHSTHSTKLSRKTSKSVAKREWKKVLSKDGFLYNRDSN |
| | | ASRNILVLGLFMALHSYRPNVFKSVRKYTDSSTN |
| 56 | PPa-3 TnpB/ | MSDSDFYSETDSEYVSESDSETNSESDSETNSESNPPPPTTCGTRHHSLFV |
| | Fanzor | GQNEEPSNRPTRYLLRQRNTNQLNLFENKFSNILKNSSISHVLRDLNEQSS |
| | | IITNHQLTLFGISPHEFQNHQLIENLIKDLSTTKSAQLNTTQYLSILSDLYNS |
| | | PDIIISNNILLSYKVNKSKIKTRELNLRLMLIGLPIIRLHPLTNDKHIIDYYK |
| | | KKINKSINVELFYEIHKKVSTFYLFLIIYNILIESKIIKIKSKSNYNDFILSFFSI |
| | | DSTPLQVLESINKANNVAYFKPKRSHQEFGHPIPGSHLSSKFVLLSEEVVK |
| | | QINKHGNKYCLEDRFIDRIRKIYPGLSSNHLWNNFHHKRQDSDPNPPESS |
| | | VVTIDSLLQIANKHSISLPKPTTREITSFKANRLCPHHLSSSDKCTKDCQSV |
| | | YGDYQWNFNMFINGITDNFDEFKKVLPSYRSTFTDTLNMGTLPESHLSTK |
| | | VNFSNRSPTLPTNTFDLDKQQHVPHQLTNVPAVHSCDSNFMPINVKDHF |
| | | DILVMTSDPISQRHLAFNKTENDICQTFKSLFINFGIPMNGSPIDIDSIHARM |
| | | SLRPSVKGIYRPSWMTRKIGDTSIYLADYAGPEFRSNGDTLSITRAVARPF |
| | | NPAVSKDKLYLPYQMPGSKREKQHVEDFRSISDTDPTCYSVDPGRSSHVS |
| | | CSSHGLVRRASVNQYYSWRSNRASKKMRRLQKRFQLFERQNPSSELDYF |
| | | NLNWSIFVEYIAFKMDPRHKYLRRINYRFGQIAKSKLVKQLIPTPASHMS |
| | | GGLVFLGASEFRHNSKGHRSFISTKGLKKLLEQHQYRVVMVNEYCTSRV |
| | | YHGTYLREEKNLMVAINDQCEHESLPKVEPTKWPPDNIPPVPPTNQPSTV |
| | | FEKVRADYAELVEFGSDLNSQGSKQEKLLNYDYYSHYSIYSTELNLKKT |
| | | QVPTRDWKRVLAKDGFLFNRDVNAARNILYLGLFMSLYSFRPKVFMSV |
| | | RKYTV |
| 57 | PPa-4 TnpB/ | MSTYKTISDLKLEDFINNETFSSKSKQESQDATNLLFHLHNIIHSFYLKNIN |
| | Fanzor | NLNNKKNLNNINSKDIYNQSDSITINYQTLQYIVNRLCNKKDRDETKGIN |
| | | KILGKGEISVLEVYKKISLENTKKQLKVFIKEYRESKVFQLLPVDQNSNYD |
| | | TTQVAKQLTTNIVNHLSVQLSNHIRDIMKLYNAAIKELLPDPEIDRTETLR |
| | | FLIKYNIASILPNKEINKLLADNKLENTDPTENIIRIINNNLDYSTLNLSKLY |
| | | SFFNGYDFEKLQQMEKGVFTNSWKFFSIGLGSVFFKFQKNIEEIGHQVEF |
| | | AQLVNSLNQHIEHSNLLQVVKDGKQLLDVLGKQEEPLLVNTSAPRDIKFT |
| | | NSESHLVIWVRSIFKNISKYGDQNLDKIQKRLSFFIPAAPTSSTTTTATTPT |
| | | TTTTKVSFFNCNSKNHEALGLAKPTPVVDRYRDSNICSTCQYHTNSRRKE |
| | | NRKDILATDIIKSLGDLKQEDNSKVNQLLVIVKDIIVEVFKSEINDNYNIDT |
| | | LIEILKIEKENNQQPSLRSIFDDNDDFEFKSDDPIAATTTTDSTNDSNNNNN |
| | | NNNNLEGSDQSYHPEETTANGFFDILNYFSRMPLPSGSSKDTSASRADQL |
| | | QDLQMTEKPISPYSLSEGPPDPDVIFVSDHLDSMVHQKRRLNENTIYYDIK |
| | | SCSWLFVDPSNSKDLSDEQLLSIKVNRIKQILQLGKGTFFLCGIALWKLLR |
| | | VLITNEEVNITNRDHWYTSMKLTCSFAGRSITLFLLSDPCHSSTITKQGFQ |
| | | HYSNWKKDTAINIIDSEAKPTTTTTISKSPHINKSNRNRRLIFINNKMIHED |
| | | KETLGTDQCMEDDVSNDTSNESIISANDPPTPTDYYKFFNIRNRSMSFTLF |
| | | PSVDLFNPFSLMYSAKDIRRIIKAEDGMTDIQVIRSILKVDFLRRIFEANPID |
| | | VHIRTNGYSGGVVFKSKSKKVQKQYELIGLKDIKDKDVLTEEQFEQLGV |
| | | VVCDPAPNHNVWLNNKHPFANGDYSFRKNVIDVKSSTHHFQSESGLNK |
| | | HKKNKLLGHDLMKWQSDIPSVNGNIFFSQDYQIFTNDITDLAIYEKRLEFI |
| | | FKSVFLPPEQRSSDIKPYGILRAVGEEKKKEQRHKSVFHQYRQCHGDAW |
| | | QKVDLDRSRSKKHSESLNSKKLFNFALQSIPIKQLTHDPMQIDSTDPNDPK |
| | | QIDSTDSNTPKRVPKLVFKGNNVPIQSDRESSQEKSGANKMKLRRVGSPI |
| | | KVRKVYIHLLREHLGKLDDGSLERFMDRFKFVLDITYLQDDFTNLQKEA |
| | | LLKFLKDHKLEFLEPIDESNVRLNDIQTAIHYYGVELIQQAMDIWKFFKFT |
| | | EPLPGNQGVEKEVHFKDGINRVEQITTTFRNQAFQKFWFRRIHFERCKWL |
| | | KDHGIKIMPDSVGGHWIRPNQSKVDANEDSIYQLLPYTRDVNGTFNFMV |
| | | TDKEDWDSTPNKKAREICKVKNIPVVSNATYITECLRQHVYLPLLSYDLS |
| | | SAANTTSSAAKVSPSKKSGSKTATNTTSSAAKVTPSKKSGSKTATNTTSG |
| | | STVAPSKKSGSKTATNTTSSDAKVTPTNKRIDSPAPSIPDLQTKKKSKKTD |
| | | NTDKKSIDSDEDKDNHPKSRKKSIIGAGCGNLNHIQRMKGVKYSPATERI |
| | | TESLQRYSFSVSKTNMKISISNEFNTSQVGFNCLISIVFGPNIDKLENDFVS |
| | | AINKPVSNEWTQAKKDEELHSLYNLAKVKPPGESKSYKPKDTKRVYNKS |
| | | FSVRLYTGKDDQGQPKDIFINRDILACTNMMAIAKAYFCGKQRPQLLAPF |
| | | TSQPIINTPGKECPRQNLISAFTQ |
| 58 | PPa-5 TnpB/ | MSHISIGPFKVKDVLKDEFFTTISSNLSEDQRRSLVQINDFLHKFIINNQDN |
| | Fanzor | FTKNFPIVNNIIVKNEDVLTNLEKTTTHLTISYNIIAYIYRRIIGSEVLESNH |
| | | MGEVWKLLQFQPINIHEITNIISEEDTKYILELFLSFKDEFDIKSKYFDEVPK |
| | | QVATNLYNFLYSQLQSHLRNLTKVINTSIQKNLINLEINDKLKKLFYFTHY |
| | | DHQNIKELGSKDFNGYKYYRLGLGGLFNKIKYTLKYLNPLKAVEADLTD |
| | | ALSLHIFFIKFFNVEFLLISFFIGLDDPSDVFIRLSNWGFDFPKLHPKMNPIE |
| | | VTFNEASIVKIAQSILGETYMDYSNVKQFTDLVEFVERLKMQEETPHTVG |
| | | EYLLCKNDNHKNVGLPDNIFKDYMRKMEIIIIFFSETVERSDLNLSSNVID |
| | | LIHQLIGLISEFLHHLNSSLPRPISSPSKAINDLKSTLKSLINQYGQDQKSIIN |
| | | INNKPYKETTTSSTTETTTNSTTETTNEESTDNNQFYSLVDQFRNIQTSTAS |
| | | PISKTYTDPNYVIDTEKYPRFVGSPLNTFSMTPQVSSPTSSIITHHPLDPND |
| | | MSLNKDRKQCATANRIQTLSMFNGIRNTNWLDLHPFVANNMSEFPKEH |
| | | KLIMLDSLQSILSDGPGLSLLCGSVNLQLLHTLIKDGVVVVVSKQSFQSSF |
| | | SLKLKILDKSVTKTITVHLTRHPCFYSKEFISAFYYFANDIRLSIKENIDISFF |
| | | YHIKRSISVLLIPQVNSFNQFSIHYNKTALGKLLNNEKINVSDIVNPKFNLT |
| | | TYEMVGIRSNGDSCYIVYAKKKKKPTANKPTATSTITTTTTTTTNETTTA |
| | | TTNNTTTETNEPTTTKTKNEIVVTKEIKLYDKVKKDPQWKELSIIAVDPG |
| | | RNHNTYLFYPGGLSDDDNGLIPNTPQQLAQMEKKFSLDMISGTHYYNSQ |
| | | SGRRKSIKNKKRGKQVDEWESKIPSLYVGLPSSKTAPTSKKPSTSRAGKS |
| | | SKDTDVVMTDLPPVKNLPLKPLQYIYKPLDPKVLNLPEVVPENYTKPEVT |
| | | ITHKLRFKGSDTKRHRDLLYLTKMAIGSEITKENVESFKKRFNSLVELGM |
| | | DFDFNVVKTFSEDFNRIQLQSTEIDQLRGKLAKHNLVFKVPQGLNDPPTK |
| | | RSVISATQRYGTNLIQQALNILEYVKLSNSLLLKCKEELKVALMPSVELSG |
| | | LCSEILVILNSKDFQTNWLHRIGYIRYCAIRRFKFDERQKTLSVSTVIPDFE |
| | | FWIKNNQQSKPINLYGKGAGPNNYVQNVKGVQYSPTLLKEIDFLERRSH |
| | | SRYGARVKTYLFDEYNTSQIFAPKLKEMEKSYVKEKSDAKRKEVYKAA |
| | | KVKAPPDDPNSYVIKYYLDGETKYYINRDFNACLNMVTIAEAYLKGKAR |
| | | PTLLCPFKKDNPEDD |
| 59 | PPa-6 TnpB/ | MFNHLIGRDSIDDFKFYIDSSPKDSIYLKDNYSPKKYLILDFSTIFYCLNRF |
| | Fanzor | LRKKEKPIDKLKFTLDNSSKYEKSYYFVLQHELNRKKSHLSEIATSTMNH |
| | | FNTHIQLLLKNIMKALNIIFKQILINNNINNISNYEIFSFLIEKRLIGFFSVKQ |
| | | YRQIVELYNKHRKLSIPIQLGIDHDILKRICLDIDSELVVALNDLDWDFTSI |
| | | QYFREYDFGQLSDLKITSKHYECRIDFLYRCCLDIVKRTTNSKMNDLTEP |
| | | YFMYRFLLEIDSVINKFFFLELKNLHRLEGQIDHIGILQKLVDFNIYQTFPK |
| | | MYNNGFYGVDSFFKISQMLESNQTPLEYFLLKDPTLMNSNFLSGWAGVN |
| | | KDHSDGSVSDGTDSEMGTLPISQDTDAGGTVSVADDSVMDTLPISQDTD |
| | | TGGTELNNAIKNIIKKSSIYKDLTNDLLFLFKQLVEFKCDAEYKIQFIILAV |
| | | QELIKHLESFTTAYIPLFYDHFTNTTNLFIHQIIQLITSKFIDNHYWFIDPTN |
| | | GNPVNPTGNDVLIVTHNPLSSVDRQWKPTQESSSSTIPPKVVPPKNYSNA |
| | | ALNLSALAWRTKTRFTLVWTELCPIVLSSINCISFVLDPILENHLIWLLETG |
| | | PSKIILTGKRDQIIFGNIVKKFNIPVHLVFQTLGFFKVLIFIGGKVKKVYFIE |
| | | HPYRMSQFLVHTFVELGIQEPLEYPSPIVETTTTTTTTAEVSSASAASATTT |
| | | TTTTTTTTTTTTTTTTTTPASTNKRMRNLFLQRDKQWMEKEPPTNSEQEC |
| | | KRSYIEKPFRKKLEDQCNSYNYSLLPIFKGLEPANLLYKPDEIHQYEPDIE |
| | | KKIKKIFGKKSIYLNHYMTDSLTLYVSFKVPTESAAKGKKNKLNCDISLK |
| | | CPMGIDPSPFKNLYLNRPLDFEFNDKDENDEPVKHCKDLLDSVMVLNSG |
| | | SHKYQIRSSFYEYKRKQVLGKDFIEYETKSPSLKCKVENTSQTNHILTLAP |
| | | YDNLIAFRYTDHYETFLEYRNQRGVQELQILNLRSLQGKKRSKNWRAAK |
| | | IFTHAIREVDRRIGEETSTPNNNSSNRFIFGNGPGHQGYVSGMKGVKHAP |
| | | YVARDMQALKQYIFTVTGVMVDQFVVDEYNTSQMFSPKLIENEEIYLKN |
| | | SSHYEKSKVEEYIKDPSWDPHSRQSESKLRYTRKMYQVNDQPVYINRDV |
| | | NGANNILLAGMRQFERPSNKNGRRFTLLMKKRGESTTAEECDDSNLVAD |
| | | E |
| 60 | PPa-7 TnpB/ | MGISLQNHFNQWLKYIECQTHHVSQLKGILFDFIQCVNRFGSFLGRPKKF |
| | Fanzor | KQNNIFIRDVESIFALFLNIFLLKNHHLIGREPIDKFKSFIDGSLRDRKDIKVI |
| | | FNEYSPTKFLVLNHNIVVYCIYRLLQRKEYVTEKIIQDKLYREKNRSDYF |
| | | NIQRDFNLKKSKQLIKKDIEEFMKRNNINNELNNNNNLFKSTTDHMTSQF |
| | | TTAIMNHFNSHIMLMLNHIMKQLNIFLKEIFNIKNNNHNNSDILRFLSEYH |
| | | LIGFLSKKQYEELVKTYNRCKTDPAFQHKINQLDWTFESIQYFREYDFQQ |
| | | VSKFQTTSKHRELGLDHLFNRCLKVVMKNKDILDCDLTKSFYLYKFLLEI |
| | | DKDFKNISNDILNDCSRILERLQRFDIYKDFPTTSHGFYGSDYFTKISKFLH |
| | | EVDRPFGYFLAMDPQLEEYSYWNIRASENCNSHQPTDEIMLDAPAQSIFD |
| | | NDTPLITQLKQGFEKRHQFFKSSKAETDFSILYQQLEKLDMPENQYKTQFI |
| | | EEAIEEFIKHLDNFYDRSTTRNQYMERTELFIQQLNQLINLPVLKHLFINPI |
| | | DGQPINSNTTPDVLIVTLSPYEVDHEPQTTKPRASKGSATVFLSKEQLMK |
| | | QDFISFLKEELERGNLDIDQLLARGVLGPSIIPPIQPSEDYLCHGGEAYCVN |
| | | LLPMLSPLIEPNCPAEERLKSETALNLSALARYMNGHCSFSWTELWPMM |
| | | VKSIYDIGYVFDPLLERHLLWLLQYQSPPTIILSGKRDLIILSNLIKNHKLPV |
| | | HVVEQQLWNFKLLVSIGGKVKRIFATNHASSLSPSLTKFFHEAVDGLYPD |
| | | SMYLSDSDLEIVPYQVVISDKVSDIVAVSDQLPVSGQLIASVSDQISGSNQ |
| | | LSELSNQLSVSDSMIIPDQLSVVSDNESDDTLLSYSDLESSDDSSDSDLVP |
| | | VQYREYPVYRVRVPDQVANDQAPTVDKPETEKEFLGRKRNLFLQKEKP |
| | | WIPRKPRSPNVPKIPRHYWKSFEKKLLDYSASYHYSLLPIFKGYELKLLLY |
| | | KPKDVDQWLNLQKLNKLCGKKLSEVNHYMTDAVTFNYSFKSESTDNTK |
| | | SSSKLTVNCPVKLKCPVAVDPSPFKNLYLNKQLEYNFDGEKHCKDLKDS |
| | | VMVLKSKSHRYHTGSSFTNYKRKRNLGPKLLEFENNVPSLKSSKENDILS |
| | | IQTYEIYIDFRYSHENYSTFAEYKLVRGGPEFTEQHLRSLQRRKIAMNWR |
| | | NRRIFNHAIMAVDRKIGEEKAKKTGDINDIININNNKNNNISTSKGSSSSIG |
| | | SSSINSNNRFSILEYEDNNNNDDDDDDDDNDDKKKMKLRGKSYYKYKQ |
| | | QEINYYNFQLALYKLYKLYPNEFLPYPPPQSLPNRKRTHTNKINDQLFPIV |
| | | EYGRSSGNTDIHELLVDIEEWFRDKFQKANGWEKILGPFLTTKSSKLFSTP |
| | | STTTTSTTTTSSATTIPTTITPPTTTSSTTTPTTPTDKSKCANRRHDSVYKDQ |
| | | PLGTVPVFFKSGKVTKTCKLCLDFIDKERKEKKKKEEEEERRKIDEDEEE |
| | | EEEEEEKQKDRKKGRKWKCKNCGIYNNYYVDEAFQDRDRVWLAHCNN |
| | | CNEIKSVFYDKEVAERYALRQIFIYGNGPGHLGYVKGMKGVTSAPYISKE |
| | | TESLQKYIFSVAGVKVDLLIVDEYNTSKMFAPKLIENEQIYLMNKSHYEK |
| | | SKVQNYIKHTSWSPNSQQTESKLRYTRLMYILEDGEAVYINRDVNGAGN |
| | | ILLVGMRQIQDSNNESRFTLLMKKKSESTTGKESVDNYLFADK |
| 61 | ACa-1 TnpB/ | MRLLCCTKTNSLTLPWSCWSTCLSATTSGLWWLARAIQLWPPLSTPSIHS |
| | Fanzor | ASWPMTSLPSPPSLWRNKMASTLQRIEEHTTIEQPWRKAEEAQQASLLRK |
| | | KARNKAAARKANKMRQMAAAAAAATTVVGGEKGKGQVGGGSRGKW |
| | | KWKGPPSAEGGGDGEGGMVAFNGKMRKICVFPMAQQKTLLKCWIGTV |
| | | RWTYNQCVAVVCRWQCAPTKKALRAHFVTEFGLREVEQIKRAKSGVGS |
| | | GGNNNNVGISWVFKTPHDLCNQAVGQFVTAYKNAVQAHGWGKFDIAF |
| | | WLAKRLQQEMVVIRSRDWNRTRGEYAPIFGNTMLRSSESLPAKMDHEF |
| | | QVTRTKLGWYYISIPVPLDVKHPIQQQQPDAVGGGDNQAPAVAPHAAA |
| | | AIDPGVRTPFMVYSPDEARVYELGANDFGRIRRLCHHLDDLVSRMTDRD |
| | | VRKKRRRRMRRAAARMRRRIRDLVDDLHWRAAKWLCETFETIIYPHYE |
| | | TSNMVVGKSKRRGLHSKTVRAMLTWSHFWFKQHLLHKIREYPSGCRVV |
| | | LVDESYTSKTCGGCGRINHGLGKSKLFWCEQCGFRTDRDWNGARNIWL |
| | | KFLTEWCNGSSRGNDDDDKEQQQQQQQ |
| 62 | ACa-2 TnpB/ | MSRGNGRGGCRASPVSIDSYWARCRFLANNTPPPPQPNKGTDWRALESS |
| | Fanzor | SWSTSLQRTMSGSWWSVRALQPTPRPTPCSKLSLTRRRSSPTTSSPLPPFS |
| | | WRDITDCALRRIDERALLPEQQPWRKQQAANLKTAQKKANRKKASAAK |
| | | TKVMEASGDGDTNQPGGKRKRKGKERAPISEEGEEEEEPAPYDGKCRKI |
| | | RVFPNRWQKDLLKSWMGTVRWTFNACNAAVRAGLSGHSEAELRSRFV |
| | | DNEAFGKPSRRKKKKAKVAAPFSERGKELLAATRHLPGPSTLWVLDTPR |
| | | DIRDQAAKELSAAYKNGTKAHGKGKFEVKFKSPKKMAQQCITSNARDW |
| | | GRGRTSVFHGLFDSGRALRASEPLPREMKHEFKIVRTRLGRYYLCVPMD |
| | | LETRGESQAPSSSDDVGAECVFIDPGVRTFVTTFDLSGRIHEFGTGSIGRIE |
| | | KLCRRLDDLISRTYAKKPDDRQRFLRGKKKRWRMRRAALRMRRRIRDLI |
| | | DEAHRKIALWLCENHRVILWPLSGVSNMVVAKEDLKQRKRRIGAKSVR |
| | | AMLTWSWYRFQEWLKHKVREFPWCRLVLTSEAHTTKTCTHCGTPNHD |
| | | VGRSEVFRCADAACPNRSAHRDHHGARNNGLRFLTEWANLPTTTTTTT |
| | | MTQRLPTRVIDLTTSGLND |
| 63 | VCa-1 TnpB/ | MLDLYKQGDTFWKDLLKNHKPLLCSEDTSYVSVKLSVLSGSVRCTTTVT |
| | Fanzor | NMMPNRTKESVKRVTNYQPGARARITVTRDKTTKEEKVTLKVTGYEAK |
| | | VTEKEVSDFNQPDGLDQVIQYAGIGFKTVVLNNVWYPLFARLGRLTKAY |
| | | MHQMCKKHHHKKTPWSKAHAVVQLIRSGKPIPAWPAYVVAYIKNARA |
| | | KLLDAAGSTMYGSLNGHALQARQDELLSAHENKYLWDDHAKKKMTFQ |
| | | AAMQFNFWMQKQFESLHQKKMRLMPVFAVKRSHVRLDTKSLLHIFCSL |
| | | FPNDHNVKLMKSFWNQDAFLEKCGLPGSAHPDTYMLPPKPPDGTTDKA |
| | | LLAQYQSACSRIRNTDAYRSQQARHRALSETEDRVVKSFFRCLPVKNGY |
| | | KFDCSISTDGVAVSLQYSKPSKAVTAKRKRSAGMRSSNVATDAYDQNLD |
| | | MYDPEEGVLTLGLDPGRCSLATVSVVTQYTLDSTGKLKLGEKLNRHSWS |
| | | LLRGEYQHKSGIRTSTIKKAQRDAPLQPAFQALAVPGCALRTAQSGTVLR |
| | | YIADSQHCMNEWWERALSRQEAMASFQGYIGKRKALDSFFSRVLKEAK |
| | | KLVPKRTKIEVAYGESGITMSPAGKGEVAVPTTGTFKACQRCFGKSAVV |
| | | PTNEFRSTMMRWESGSKTHAVYKEFRAAEGQDCVKEVLHGTDQKHGSA |
| | | CQHPTQGFRSSWPGKQGGRARTSNAEGPSLELQRRSSSQQLAATLHSQW |
| | | FRVTRMYYNRDTHAASTIGRLRIMERKGLGRPAPFCREAKA |
| 64 | VCa-2, partial | mvlaepficlsafkvgritgrwaaftrtmdtdgtaacfhfrvrraavgpavaravaargvaa |
| | TnpB/Fanzor | rstqrcsehcwREHPDVEVVGVDPGCTNIIYTVKRVAALLQPPLRLQRPLRLQRHkvykltr |
| | | kqyymesgvvkantqsaawqslpghaagagdtgcggrskcvdaegfgavrrcgrgapeaavg |
| | | gQRLRLYGSKECVFARFFQRLQDGTQAGSKKKVIVAYSAVKFALVGPGELAVPTSRAS |
| | | KECCQRLVTFPVDEFRMVAVWNGDKATVLQDFWSQSLMWQKqapykdgp |
| | | smlcrisstrltNLINKEALSQLLGQVTEAAEHSLHIRLHRVVIGILFVNEVLQEH |
| | | AGDRLLVALLHAHHFWVRDGRADVLQRLQALLKARVLAHELLEHRLV |
| | | DTLVATIGQVIECHVCC |
| 65 | CRe-1 TnpB/ | MAPKRRRDEAEKAEEEKDHTTSTKCGLAGLLSEKIEADGVAVTREESLA |
| | Fanzor | AVDFLVAALTRLRFEALCLLGLVAVRMCEDARREGQGLQPHCATCRRL |
| | | RKTELVEDDMYAAICAVSVCDLTEQGRKRGRPSKRDQHPEDDLFRHVCE |
| | | EHFPRDEEAAGARVNRSGLTPFLPPLSKGVFTNVKNHYAANFAAWLARS |
| | | FRCRIDDELRELRTPATKKLDKLAWSMAHAVLYDGELEQPRWWVGWA |
| | | QGAAGAAAAAAAQGAGPAGGAAAAQAWTALVDYVNAQRASKRAAEL |
| | | LLREVKGAQATYKKASTRHMEWAAEILAGLEARRDQLGAQVQQLTQA |
| | | QPLTREDTQRLASLRRELHRARPFTLTPSPSFAPIYVPLDNTSMARLPGLL |
| | | PTLARRHGEVFAGAGAGAVAPSSFVQAAFGGGGMQSSATLNAVGWGLF |
| | | QLGGVTSRNAPFANYITTDGVACSVAREAHNKPLANLKPATAPADAEEL |
| | | CTLEEMKATQIIGVDPCGGGNWFMAARSPLYQPGPWAWEGVGPAQRYL |
| | | LELHDKQLDEELFPGQLPPEPRRRRKGVHRRKQSKHWQPRARTARRRRQ |
| | | KRGRFHMSMGHWRHMSGLERLQPNRPQLAPALQAYVGGIPTAATASAA |
| | | RFEERLRYLFASGAAGQAAGGPAEAGPRGAVHVLWHYHFSAFRRKRWA |
| | | AFIQRDRALHRVAKQLTGGRPKEEVVVGWGSWAFQGGKGGSPISVRGG |
| | | RAPTGRLIKLLRERYAKHVFIIDEYKTSKTCYNCGCQEMAIKRLGGLKEG |
| | | QRPWSVKVCNDCLTTWNRDVSAANVIRVLLLLKLMGFERPTKLQRPPW |
| | | PPAAAGPG |
| 66 | CRe-2 TnpB/ | MVQKNSTRKVSLRSVCKDEATLAAIRFKVDFVAXPLRYRLLDFLRHFVV |
| | Fanzor | CRTDPAFHCAVCDTRWREQCGLAARRQGLRERADVSPQQHVFHRRVPTI |
| | | DINLLQESAALITTSTHRRQRQSLLSHFYDAHFAPLLSEDTGGPAIDITAH |
| | | HQLVHHLLQDVLTNLEQHYRANFPKWVRKYCRAKLEVNDDEVPLGGL |
| | | AGMALGQPQAGNEGGAETDEEDEEDQVHVTAKARKALAGLMTAALM |
| | | GWRQLPLTEADLGGLAVPDALLARANATVAPLVAAWTARGIWPIQRGS |
| | | RGLLQREPLCLVSAAADLLCGLQPIRRWLATVYERLKTANPPAPPGEVRS |
| | | ANFAMKRAQTYALIIEASSMPLHIPLTNTIIQELLVNHKQLAAAEYAAAG |
| | | AELLGSNATAVWQAATAAAKHAGGWKAFANVQQAAGAPAHHDAWR |
| | | DCGFNFDWLKRLERGGGTFAYFMRTDGAAACVTLTDAAGSSSYLDGPG |
| | | RAPGNNPVPGVVAAGQALAQYTPDALKAMRVVAVDPGCVNYLVACVD |
| | | YLADFSPHSHQHPAHAVRLWAGPRDIPEAERQQLAQGPTRPLRERRRER |
| | | AKRRRRQRRLRGVGRANVWRKRQRGQSRGRGRRGCARGRWRRKPRG |
| | | WRGRRSRKQRHAATKRRREQRKCYARVSGAGYRKRTGQSERQRWQEA |
| | | RLQEQPALQRWQAGIPSARVAYAADHTRRIQYLYGGSGGIGLWQLLRH |
| | | YRQWGQRRWRLTVYIRSQKVLEQTAQQLAGGRPKEEVIVGWGNANTG |
| | | HGGCVSRSGRGPNRALLRLLVDKYAHLVVYVDEYYTSQVCAKCGRRLL |
| | | GNGQRCLEVVIPFGGSRARDVQVCQHCGTVWGRDANSATNMRHALME |
| | | MLLGKPRPAALRPAGAHVRSRGGGQGHVEEDSAAPPSKRRRRAG |
| 67 | CRe-3 TnpB/ | MTNRISTRKVSLRSVCDNDDTLAAIRFVVDHVATPLRVRLLDFLRHFLVA |
| | Fanzor | MCDPHWHCEVCRQRWRRESGLAPEGHQLRDRSGVQMLPRKQRHNRGH |
| | | PDISLDMLQEACALVSTSTRPQQRESVMAHFYAMHFAPLLVEAACGPPV |
| | | DITAHHQLAHHLLQDVLTNLEQHYCANFMRWVRKWSRAVLEVPDEQLP |
| | | PAPAADDDDEEEEDDDDEEEEADAGARVFKALASLMTAALMGWRELPL |
| | | TEGDMGGLAVPAGLLDRANVKATALVQTWKARGLHPVQAGQQRLLDR |
| | | DPLLLVSAAADLLRDILPRHRWLVSEYRRLRTAAPPPPRGELVKAKFAAR |
| | | RTKPFSLVVEASLMPLHVPITNTVLQEVLINREVLVVAWEAAAAAERDN |
| | | GGAAEVHKAVQSAVESGWRAFPNSAASFEPWLQRQGLEPGHDPWLVA |
| | | GFQTRRNAWDGWEFAGFLRTDGAAASERAGVAAAAHSSPSRGRAGSRA |
| | | RGRGGTRAHNSPNEDGDQRGPRGGPGRAPGDNPVPGVVAAGDPLAVFE |
| | | QATLSPMRIVGVDPGVGNYLVACVGSMPGFSPHSYSGRPRAAHVWRGP |
| | | DGQAPGPAQPPLQRRRRRQQRRWQRQQRRQRRQQRRRRRGQKRRWRR |
| | | KGGGRWKGKRRGWRGTRQHTHQRAATERRRQQRACFTRLSGAGYRHC |
| | | TGQTGRRLWQQARLEEQPALKRCQAGIPSARVASAAEHELRIRYLYSGG |
| | | GVDAAAAAGSAPAPVQQYGLWHLLRFYRQWGQRRWRLTVHVRTQKV |
| | | LEHTAQQLAGGRPKEEVIVGWGNANTGYGGCISRLGRGPNRALLRLLVD |
| | | KYAHLVVYVDEFYTSQVCAKCGRRLLGDGQRCLQAVVPWGGSRAHQV |
| | | QVCNHCGTVWGRDVNSATNIRHALVEMLLGHKRPASLQTGGGGGGGG |
| | | GAAAAAAAAAAAAAAAAGARARILVAEGAGKATWRRRAQRRPRSGAS |
| | | AQAEEPAGV |
| 68 | CRe-4 TnpB/ | MTSPNIHFQFLLIPSSDTHGRPAIGEKFHFEQARAAQERFDWGRGPRLPPR |
| | Fanzor | RVLEACGEAPLSRDCSQGTIKTTFKSIADPPGLEQLLHAQHPTAAAADVQ |
| | | RLASQHRDSAVLLQKALQVLVWVMTAVRQEAFDLLLLVVALCAEDTRL |
| | | QAAGQPPNPERFRGQSSIAEGFRRQRQRACGSDQPHHVINDAAQRALRG |
| | | FPDLNQRVMEDCITAVTTSEHHQEVQHSDQRAGRDPHSHNLAQYVYGH |
| | | VFQVHRSEDRVGPPRSVTGLTSVVHQLARQMETNVGVHLATNLFRWLK |
| | | TRCRVQLHRTLHPEAAAAAPDAGAGGGAGVELPRQQQTIVLALAAAIAR |
| | | DEPLCAYPDAAAALQSQPDVLQELELWVQQEQDRWRPRGLLVPGCSFK |
| | | RHSLPHGERYLAWAAEVLHEIEQHRSQLRARGVPKAKVARHFPVFGLLP |
| | | EPSYQQVHIGLGQEAVKELLVHRDHFASEHHLIPPAAVPTGKFRNIPACF |
| | | KEWVADHGLDPGDVWAAAGLRLGISQHEHRGSKFDGFVTTDAFDMCLT |
| | | MRRRYHSSWDEAKEAGKQVRVSEEELLRLSLRGVDPGCSPIYWTCAMR |
| | | WPWLPAWVVALLGVDLVLRLPEVRNGSCPVRWPDDLTKDQQAAAEAQ |
| | | LAAHLTASKAAAARDTIVTRTQAADRATAAASTAAARWAKEVQAAPQ |
| | | QGRPELVPPRPLALMVAQSAEHSKTHCSGAEHRTNCGYAQHQRWQQW |
| | | RVKADPALCAWQSSLPTARTVDVQNVLGRLDYKYGGSTGAGGQQGEA |
| | | VVRELGFVDLLARYAAPAWRQKRLQVYCQKQRVLCREAVRLMGGEPE |
| | | VTQQNGVVGWGGGSAGFHGTVSRADQPPRKGFVRLLRERFARHVIMIN |
| | | EYRTSMVCANCGRLSLRRKEPPQGGADFRVLVCNDCHTTWDRDENAAL |
| | | NMRLLLVLQLLGRDRPAVFCRPEGGVD |
| 69 | CRe-5, partial | LPVSVLPDLLAICHSFSRKSVAANPAHISGMEWRHAPILRGLRRLLDEVA |
| | TnpB/Fanzor | AQLAQNLDQHLNGNLFRWLCRRCRIKPASEDLVQLVRDERDKAWSGRG |
| | | RGRQHLERGTDFAIWAADVLADLAAALPQRQAAVEAALQQVQQLPLPQ |
| | | QQHGVAAQQLRAARERVKDAELYALSPQPSFMPVHIPLTTSILRDDILPR |
| | | AADLARLAARIPQAQLPQGAHIHPQHRWHSRASVAAGGVVGQPGHAAG |
| | | GGGGGGADPWAWLGISTGVKKLQTGSWRFANYATTDGVDFCVGMHRP |
| | | ERPSDDDVKEEEKGLRLTVQQLLELCLFGVDPGGLLQSGAITILERDMDG |
| | | DLVEFVRVEGPADRLQRQLQQYRADRKQQKQRKRRQWRQHGSRWKH |
| | | APGGRRRPNGRQQAATRARRKQRSRFYSCSGAGHRQQCGYNDRRRWQ |
| | | EWQLNANPALATWQSNIPSARVLTVQEHDLRLRWLFTGPGAAPPGPGPG |
| | | GGGGGGGGGGGGLPLPPPLPQGPRLGVVDILVHYFNPQQRKKRLEVYIR |
| | | KQRVMHREARNLTGGRPKQQVLVGWGNGRAGWNSPISRKGQPPRKEF |
| | | VRLLRDHYAAHVVIVNEFRTSRVCFECGHMALQARLKPGMRRAHGVQV |
| | | CNHCGTVWNRDVNSACNMLLLLVLQLLHGDDGRPEVFGGRPHADADA |
| | | DG |
| 70 | CVu-1, partial | AAQAQAAAETSAKLQRFTIVTGLLSVLQNRALRHPINEAVRDTTFVKYET |
| | TnpB/Fanzor | LTLLGLYVAHYCKEVRRRAAEGLEALPAMPYLGATFISHCIRLVTHSDEA |
| | | PRWNQLPPDELLLSVYHHLYLPSRSNNLRRVNRDRRSEQLHYLAIQLAV |
| | | NVQLHVRLNFQRFLRRWCSMRLYQDFGEHQPSPRGKVLDRMVKSICAE |
| | | QCLNTDLPEGLSAAQRAWVQALVTSQRAAHTTLLPADARELRRQGQKW |
| | | QQRQRQQKQKQREEREAAVKQQAEELRNNPGIPAPPPRAQELRRKRKK |
| | | KKKKRQQQARVEAAAAPTAWRQPRVAPFSLMPVAAFTPCHITLNSDGL |
| | | RVGAANALKGDTSRVLSLQAMVNTLGKKTKAAGGRALGEEHKQQMLL |
| | | RDVELWDAHFDMWPLCTVNHKFALFLTTDGTQASVHMKRPARKPPAPP |
| | | SFPMSLVGKQLLGLDPGRRHFYTAVSDSDSKEHRDVRRCSTGHYYERAG |
| | | FTARQRWMERRLAAAGLQGWQSRIPRHATADLAAQQQRVAYVTERLD |
| | | ELVMHYCSAPWRKKKWSVRVQKQRVLANEVRRLTGGRPKEQVVVAFG |
| | | DGTGGPGGCIKGSRMPPIKQFRQLLQRFATVLVIDEFLTSQRCPRCEAQY |
| | | TSRVSYAVPVCNHCGTHWDRDVAAAKNMLRIARAEAAGLPRPADLRRG |
| | | G |
| 71 | CMe-1 TnpB/ | MEKEVEDPGGPVSPAAGKKRKTVPSPEEQEKTKKQKREARRQRLQERR |
| | Fanzor | QYDEMVVKACLLGHIKDPYKEKLREAIRNRVESYSMSIVKASSGLMHLV |
| | | REMYRDVTDIETVEVPEEFFDKTFIRHLMLGTGETSRRNERVHALHENYA |
| | | EYRFNGTRYRGDSDIYAYGAMKYLTNLKNHLTMNLERFMIRAVFALYP |
| | | GLSRKGIWAIINGITNDRXHEQEIEFVDKKTSRRSTNEASVIRAAIQEHRA |
| | | VLGLANPAEKVSELKKDKERCYRLILRYFVFLDRELERKAEMELSEEKNE |
| | | ESERRKAALMGKRFNVVPLCNIKSHFVTIDSRVLHGIMKEISPEFDVSREE |
| | | FTGENRETYWKNIFDFKRLKASKQKVFTGMIETDGVAMCVHYRRLKAD |
| | | RPVPPSASPVAKHEEKKEADPATQEVQENDFVVGADPGNTNIITIAAPKR |
| | | AEDGTDGNLRQKDMRLLRFSRARYYRESGIMNARKKIETWNAGMKEHL |
| | | EAMSEVTSRGADFQAFREFMEVRVAHWEALWEEYTKPRWARLRMNLY |
| | | CGKQRAFANFFNQLSALKEDESQRLVVAYGAGRWTTQKGTTPAPTTRT |
| | | YKECARRFVTIPVDEFRTSYTHHELGCTLQRVEMEKCQRSPEDIEKYGAL |
| | | TEEQMERRAKVRGLLALVSTTSDGKKRMEFVNRDFNAAINIRRCAVLEK |
| | | RPPELTRENFVGQPLKVELYEKKLEAVVGGRSKKTGRRLHVSWRRFV |
| 72 | PUI-1 TnpB/ | MGRKRKKDDYPEEWDVVDETQDPDAIPPLFEIVAETQDSDAEVDVVTN |
| | Fanzor | ALSQLPPPSQKSLAPPTYRIKETTKEINSSVICKLTRICKFPLLVGEIKKVCV |
| | | AMKQVQLEGWHLANLHVLRCLKEGDEVPKIEQMFFYRCCTATLGNIEK |
| | | RDRGSTESKYPAFHKTCLRYWAGRERESTYEPEFIQTAGDMVNEMAKL |
| | | MEINALNMIALHFRQRLHQYIQFRYAKKGDTKLKYRETRRLVDSCYRVK |
| | | SEPEKDDEGNPTGKTAKVWTEWDDTDDPVEQELREWLGIAPWQWQIRA |
| | | NPAHFLHKLYDMLVWMEKFVDKHPKTKGARVYSLLPVATTFQAAYVK |
| | | LNASTLHGLLARLIDLPEVEDFLKKELNIVPTRKRTGSQLPFDKKTFQKNR |
| | | SEVIRKVFDVEQFETLDRKFADEIKTNGYGASITMIRPVTTVSVALVEKK |
| | | ASKKKRMKNDGTAELVSKEKTARKKKVKTPEEIAEEDTFAKELFKLGPD |
| | | YSPNVLIGIDPGMRSLATAVSVGRLQRRRRRQKSRRGLHRRHRTRRARR |
| | | KERVIEISTPKYRHLARMNDFRFYNENLKMREPWYAGVIRAMPSFKTSS |
| | | YDLYFQRLQFFWKHLRFLLVFSAEQAFLRWRFTQDRAKMKALDTLAKRI |
| | | VPKANKQVCIAYGDWSRRDGIKGHASGPVKGFVEALKRRATVIPMDEY |
| | | RTSITCSCCHQRLKQARLFTKMKRKEDEVDIRQKERPSKKEVKEIVEMAK |
| | | FKNPKLADKKVVLKCTRNVLRCTNSKCKANFWNRDVNAARNMLELLK |
| | | SGLKEKHGARRLRAFRRGQ |
| 73 | PU1-2 TnpB/ | MTGYKEGEFFGGFSSASSAPNNPLSNPPRAMKSAGKRKRRRKPEKQKRR |
| | Fanzor | RPDVIARRVANADTAVSVVKTTLNSFCKEEGKALSWESVLRDMNKAVL |
| | | EAYVLANIHVVRLRQASLPIEPLNQNFFHRCMTAVSSGIRESQDCEQLKQ |
| | | SVALYNEWRASDVSRANIEYVARGWQQNAAKMMATNATNAINLNFYR |
| | | RFHKFLKRKYGFNGRQAYATLKSILDPKYKEEDPIVIEWRARIPRKHDGK |
| | | LDCKPHVLLPLTPLTLTYMFLSDIEERNQVNKTTPDYVEIRSFSLLPYKRG |
| | | FECSHFKVCKTGLRALLKRARVDVPYEGAGWNEVADNWWRVFFHIDKF |
| | | ETANRKFDGEIVTDGKSVSIVMRKPKRESVTSELNAKDYDVVWGLDPGR |
| | | TDLFVATNQYGEKIKCSSREFYDHARYKKRNQKIKIWQENRPAVLEVIRN |
| | | MPTKKTAQLERLKEYVKFMIPRMDLLLKFAMVKPFRKLKLRSYIFAQKK |
| | | LRELCLKLTAQGGRRTIVGFGDWSNQDIGGLIKKCPAGPVKPLERMLRK |
| | | YCRVESIDEFRTSKLHNDCHRSLDHQYSKRKCRDGKERTLKVHGVLHCK |
| | | NNGCHGMTVNRDVNASKNMLLLLQLKMRGEDRPPAFCRASTQEEEQQ |
| | | VSTSAVS |
| 74 | PU1-3 TnpB/ | MSNKRKKKKEDPDNPDSWDIVPDSQELEDVYSTDFDIDDGGEESLSQDL |
| | Fanzor | FAMTVTEAPKRKKTRTKKVTAKSKKTQTRTGKPQRKKSPGTIKEKEFKK |
| | | GKAYISVVCRLETICLDSRLCDEIRRSAHAMKQIQLEAWHLVNLHTLRCL |
| | | ENGLPLPDYSNKTFFDHCCAGVSTTNKTHHIAQKNPDLWESIKIYQSQRE |
| | | RAGLSEVTHLIGYGDLKYELREQMVVNAGVMIREHFRTRLRLYVDITFG |
| | | GLGDTLTKKQKKEKADLVKAIMHACYSVDETDVVEALQMRDILTPDGV |
| | | GWSEQWIPWPDRIKENGMAFYVRLLWEFQDVVEKRMEAVPNEKGVQA |
| | | FSLFPVSTNYSRAHIKINGTTLAGFYSRIQQRVPEYRWDLPSVSLSVRSFK |
| | | ENRWTVMRRAFDIAQFETRLSECPLSKAEFAQLSVEEKYEHASHLFANQ |
| | | VTTDGYSASVLMYRPMTEEEKADKESEKKYEEKADKGPVVPPGYVPNV |
| | | MIGLDPGMRAICTAVREDFRPESQGDRPPRRRGRRQPRRSHRRRRSTRKK |
| | | KSDRPHWIKRPKAPRHRNGRDIVSVTTREYRHLAGFNRCRVWNEGQKK |
| | | RHPEYQAIITKMPSFKTASYETYLERLEYFWRHVRFLMQFCVDRPFLKW |
| | | KFFRKRMARVAVDEIARRIVPTVSRQTCVAYGNWSRHKGIKGHAPSPVK |
| | | GLKEALRKRATVVSMDEFRTSKLCSQCHQPLSSVSYSVNTMLPKRKKRK |
| | | GMVLARNRAEIEFEEKECYAVLRCDHKDCEARYWDRDVNAAINMLELL |
| | | KSEVLGLGRMEAFQRP |
| 75 | PU1-4 TnpB/ | MKSAKRKRPPEDSDSDFEEGVTPRIASLRLRVAPTDEIVQESDRGSSCDES |
| | Fanzor | APAVREVIILDSSDEDDLLEDIQTMTVRPKSVKRATTRKNASAKKPKKVS |
| | | AKTSQQQVRVGKEPAARKKSSGTVLENAFKKGKQFCSITCRLETVCPDP |
| | | DLGQQIRDCVRRMKQIQMESWHLVNLHTLRCLEEGIQLPLYDETFFDQC |
| | | CSGVSSTSRSHNTANKNPALWETIKIYRAERSRTNLKEVENQVGLPKMK |
| | | YELRQQMVANAGVMLREHFRRRLCAYVKVVYPNAKEKDVLHGCYNSE |
| | | PTKLEEVQEMRALLTPDGVSWCEEWIPWPNRITDDSKIEFYIRLLWKFQR |
| | | VIEDHIEANPGRSTKGVRAFSLFPVATSYTASHIKINATTLAGLCSRVKKK |
| | | DPETGGDCSITKWWGVDHIKIDHNAFAAEKWTVMRKAFDIDAFETLRPD |
| | | TDVSQADFRHMTVEEKYAHASHLFANQVTTDGYGASVLLFRTKNELDE |
| | | AEKQQSERLVPEGYRPEVVIGLDPGMRSVCTAVRERFVPGQIKRVRRRSV |
| | | RKKRRRKRSWIRRYDGPKRKNGRDIVEVHAKEYRHLAGFRKRRTWEEV |
| | | LKNSNPEYGTAIRGMPSFKTSRYKVYLERLHYFWKHAGFLLEICWRKPF |
| | | LKWKFFNKRSSRIAVDALAARIVPDPSVQTCIAYGNWSRRDGIKGHASSP |
| | | VKSLQQALAKRAKVVILDEFKTSKLCSTCHRELSPVSYLVNPDLDKRRK |
| | | RAGKIISRYHSEERKLVECHAVLRCDQKKCEASYWNRDVNAAINIVNLL |
| | | KYEIEHGGRMAAFARVQDRRD |
| 76 | PU1-5 TnpB/ | MKARQSNKRKRPPDEDEIVPDSQEYEESALEEVVSDLSHLSTAPVNAEIK |
| | Fanzor | PKRLRPDVTARRAMNTGTTFSVVKTTLNSFCKEKAKALPWESVIKDMNK |
| | | AVLEAYVLANIHVVRLCQANLPIEPLNNIFFYRCLSATASGVQESRDCEQ |
| | | LKQSVALYNEWRGTGVDRANKKYITDGWQQNASMQMATNTTNAVNL |
| | | NFYRRFHKHFKRKYDFDGRQAYATLKHILDLKYEGDDPIVLEWRARIPR |
| | | REVDGKLNSQSHLLLPLTYTFLSDIEERNRVNQTSPDYVEIRSFSLLPLKR |
| | | GFECSHFKMCKIGLRGLLKRAGINIPSKGPKWNAVSDEWWRELFNIDKFE |
| | | TANRKFAGEIVTDGKAVSILMRKPKQKIGTRELKEEDYDVMWGLNPGRR |
| | | DLFVATNQNGEKISCSSREFYEDARYKKRNQKIKVWQENRPDILEAARN |
| | | MPTKKTAQLERLKEYVVFMIPRMDLLLQFSIVKPFRKLKLRSFIFAQKKL |
| | | RELCLKLTAQAGRRTLVGFGDWSNQDIGGLIKKCPSGPVKPLERMLRQY |
| | | CRVESVDEFRSSKLHERCHTPLSHQYSKRMCRDGIERILKVHGVLHCKN |
| | | NGCHGMTVNRDNNASKNMLLLLQLQI |
| 77 | PU1-6 TnpB/ | MSSHAMQAQVSIALGGLIPQLAAMSRKLKRKRSDEQGETDASEEAKVA |
| | Fanzor | KSQRPDVTARHAANANTEVTVVKTSLNSFCKDKGKALPWEGVLLDLSK |
| | | AVLEAYALANIHVLRLCKAQVTLQPLDQGFFYRRTSAVAVGHSGVVRTS |
| | | RDCETFQRSVAMYNEWRAAHVERACNFYICAGWQQNASREMATNASN |
| | | AVHLNFYRRFHKFLKIRYDLDGKQAYLMLNDIVESEYEGEDPRVLEWRR |
| | | HIPRKHNSKVDNQPHLLLPLMYTFLAYIEEHNRLNQRTPGFVEVRTFSLL |
| | | PFKKGFVGSHFKMCKTGLRALLQRAGQKVPGAGSEWNAVANATWREL |
| | | FNIEKFETASRKFAGEILTDGKSVSVVLRKPKRKSTPCNINPADYDVVWG |
| | | LDPGRRDLFVATNQLGDKVSCSRREYYEYAHINESNQTIRHWQHSRKDV |
| | | LEAIRNMPTKKTASLERLKEYVDFMIPRLGLLLTFSVVKPFRKLKLRRFIS |
| | | MQKKLRKMCLQLTEGAGRRTIVGFGDWSNRDVAGLIKKCPSGPVKRFE |
| | | RKLREFCTVVPIDEYRTSKVHADCHTPLVYQYCQRLCRDGVERRLKTYS |
| | | VLHCPHNGCFGMTVNRDANASRNILQLLQHQMQGTPRPQVLCRGGTTK |
| | | STSVHGCPV |
| 78 | NOc-1, partial | MLHWYLSRPVEEQVALPKLDQSFVTACFTPGLEVRPGRAWNEGYRRVIE |
| | TnpB/Fanzor | EVFQDKFTNYPFIHRSPGDYQSITVVAQLYITNITASTWTPFVGRQLDYVR |
| | | FWLREHGLESPDVSAKRVQCLINGWITPRTDNQAGAGGGGTGGGGGGG |
| | | ALKVTATARAGASATAASTAATAAAARAAARVAAARAREEARARASA |
| | | GGRCGKGGGGGGRPVGQIPDEVQDFINEERRLLRLEGSTRLDVNEKTPV |
| | | EGLLLPYLYHILYQYHQMHPAVLAVQEGMGDKPEMQAEHGGNPGYRA |
| | | FTIAPICSIKRHFMTLDTRALGDLLKNVMRRRPTAMTKEAWSEACPDWL |
| | | RRLGGMGPKRFCRDAVSVKEMWDETFQVGNLRNQRVFHNRVSTDGVS |
| | | VSVFFHVAKERLKREKAERVRVEKEDEEKEMLERQQGQENLQPVRERRI |
| | | VAIDPGRVNLVTALDSQTGKIKTLTRRAYYEKGRINECKRKFSSWEDSLD |
| | | DINTAFSRTTMKSCCPDLRSAHRELIVRHYHRLWDARCEKKRARAHMA |
| | | VYAGKQKVLDGFFAGLTGPGQAKPIIAYGAGIFSHTGRGEVSVPVKRVL |
| | | RVCEKHYRTVYVPEHLTTKVHLDYGQRLNPIARRA |
| 79 | PSo-1 TnpB/ | MIVPATVLAAAVGKRKRGTGSSRNQQRQAKRVARRERNQAAAKKRKTS |
| | Fanzor | SGAVTTKRTPKTTPRKRKTPVKPKPTWTFTTVKMKLRTFCKDEGVALA |
| | | WDSVLRDMNKMVAEAYVLAAVHVVRCCEQGFDIPILARSFYQQCLSAV |
| | | SGGNLHDRRTTMTEEFRDSVAIYSAWQGESLIPNRDAINRGWAQVAAER |
| | | MAHETQNHLQLNFYRRFKQYITLRYGLAGRDRYLVLKGILDPEYDGDNE |
| | | IVLEYRELIPRNDEGASXERASELLPLVHMFLTFVKDHNREHEGEPGFEM |
| | | ARTYSLLPTKRGFVCSHFTMSSRGLRAMLVRSGYDVPEEGSDWNAVAD |
| | | SMWRRLFNTNLVETSKPDGSPSRFFAGEITTDGKSVSITVKKPVRGRQEL |
| | | QMSPETAEFFSTRWGLDPGRRDXLFVATNNHGETIRCSSKEFFHAATYTQ |
| | | SNKTIRHWYDYADEEVSEANRLVPTKKTCSLERLEAYVRYVVPRFDMLL |
| | | AFTMRKKFRDLRFRRYCQSRKKLHELCTRFVAGRDPARTIVGFGDWSCS |
| | | DFGGVIKKSPGGPTKRFMRELSXYCRVELESEYRTSKVHHGCRQAHPGD |
| | | LVTQYQRQECRDGVVRNRKIHSVLHCLRNNGGCGITVNRDVNASRNILR |
| | | AFELRLSGQPRPPELSRSSQQRST |
| 80 | PSo-2 TnpB/ | MERRSCYSRSSLTTSKSRRMPSKKKRARPPGSEVPNRSKARRKASKKRAP |
| | Fanzor | PPDAHDTGGPGPPKRRRPDVAKRKADAKTVDIVARKVTLKSFCTPEARA |
| | | LHWNDVIMTMNHAAAEGWLLANFVVMHTIENPTGGGVLPPMDDKFFR |
| | | RCMATVSKKSRPKKTFKAVFQKSKERKVQPDLALEEWKWVDFERAADE |
| | | YLKHRGGARPLDCKYICLGWFEQAATQMAKNTETSTWKNLARRLRQY |
| | | VRVKYPEHRADAFLITSWVLDEACNRRHCKSCRTKKRQKKPVPAECQSC |
| | | DAVNGIVGALREDIPRNQNQKVDWKSTDVLRLFHKYLKAVEEFNETNK |
| | | DNPKIRQERVYSIIPTKQGFRSSYFSMTSTGLWDLLRRSRDRLDLDTIVYT |
| | | EGGGSISLGDSIKKENWSGVADIWWRKLFRVADLEKGTRLVFHGQICTD |
| | | GIGASVLLHKPKREPKAEPPLRKIENYDEIKGADPGFTDMFVACTYNVRS |
| | | QTFSDDFLSYPAKKFYSDSGYSSSNRKIRRKLGHNAEIKKVFKDMPTKKT |
| | | ASLTKLMEYLDYTVPKMERLFSFFMTKTFRDMRFRRQMLAEKNLRAICR |
| | | ELAGEPGTRTLIAFGNWELNNSSGIIKKKTPGPVMKLRRMLTDYRGVDV |
| | | MIVDEYLTSKMHHGCYSLAPMENQQVKHRCRDKVVRTVGVHKVYHCL |
| | | KRNGGCGATVERDVNASKNMVEVALTQMREGEGVRPERLQRPS |
| 81 | PSo-3 TnpB/ | MGDTAFGGVWYRSRLAQVGLIPQFARYRVSARSPPPPADSTPVYTYQSV |
| | Fanzor | KMKFNQLCDLDFLTAEIRTMASELKLVGLLGWKIAGKHVLRCLQEGASL |
| | | PALDSNFFYQDETEKRTRNSPEYPDLHPTFVRYWELCERLPEYLPPSSVNS |
| | | GPMRRAMADTMETNTINMLGLHFRRRLHQYIRFRYATTGKLQLGYKAT |
| | | RKLVAGXYRVKEVPHLDVDGVDTGNVVYVWDEPRSTAERELHDWLGF |
| | | VPWEWRIRQNPTHFFVKLYDMLQWMEAFVEKHPKTKGARLYSLPPYSS |
| | | SFIPAHVTLNITSLHQVFCRLFTDPERQAEVKTYVKDELGVECGSTEQVR |
| | | RQFSENKDAVFRKMFRLNGLETSTREFAYQITTDGYAASVLLRRQTLCRE |
| | | DDDNADQPRKRQKNNKGEVVKAPEAVEEDALEAAMKKLPPGYAPDVM |
| | | IGIDPGVRTVCTAACEGSLPTRQSRRRRNRQGKRKHKIRRAKHKRKNRH |
| | | RQRTVAINTREYYHMSKAGKHRFWNNGLREKEASYAKILDGMPTFKTA |
| | | RYQVYLDELRYFVKHAKYLVLFSARHFFRKWRFLLYCMKMKAIDTLAK |
| | | RLVPTPSPQVCVAFGGWSCQDGLRGNPRDPVKAFRKALERRATVLPMD |
| | | EFRNSKLCSCCHQSLRRARLLTKVKDKDTKEDKIVLKGNRNVLHCKNSR |
| | | CRAHFWNRDVNAAINILELLKCRLLDLARLAAFQR |
| 82 | PSo-4 TnpB/ | MEAGWHFQISLQSSPPRTSELAPSAMERAACRPPGRLRDWKRRYACSTV |
| | Fanzor | RCKLGRVCPHPELINEIQICARLILKSTTEAWALANFHVLRQCEERGDIPD |
| | | LTSSTFSPGAVESSAGAFAEDDVRLGDLYDSAVAFLGARPSTYTYEPDDV |
| | | TKIDVLHGEAGGLMSVNATNMVVTQFRKHFTLFVRLWYGISAKETRKV |
| | | VRACYSSDASERREVLEMKAWLGSAAWGEDGELEQVFPRKTVIKKHVG |
| | | YFVRKLYEIMVFVEQNQIEQRRQRAQSMFSLLPLSQSFALYNIKIDPSTLA |
| | | HLCSRIYKRTGENVLGVELTPNNRIPTRPYFTQRGSELMRRAFNITQFETR |
| | | RTKIGKARFNGLSVDEKARHAARFFGDSIRSDGYAASVLMVQLTRLARS |
| | | VGRREENEEDEELGDADEFDDDPDSESLVTETNGEEITEDEPELEPEEDTT |
| | | HEQDPSIASEVSDSAPYPAPEPALRGIRARGFVGVDPGKKAPCTAAIIGKL |
| | | CSKKKKPPPPPPPSPKPPRWHRNKRCPVEDRVSSTGPPRTPVRRRREPKLP |
| | | KVQHEQRILQIKAGEYQHMSGSKKFNKWHEKLRLRFPEYQIAISSLPCLK |
| | | TADARVFLVRMEQVWLSLDYFLDFGIAHNFRKWRFFSHVKHRIAVDDV |
| | | AKWLIPEAGAHVCVGLGNWSQSDNIKGGPKPPLRPLEKAMRKRATVVK |
| | | VHEFRTSKLCSACYQPMKMAVFLDPDDPGPDELDADERPLYYDRSVLRC |
| | | ANKNCKKNFLNRDVNAAVNMQTLLMARLQGLPRPAEFVRQ |
| 83 | PSo-5, | MTAVLASRTQFFRMKNEGKKTTFPVLGFRSERARSNSIGIRTKTEGFELV |
| | EGZ05744.1 | DDGGKHYVQFHSKFFGASLGKGIRLSHAVPTPEHMARLIRVRGGKYYFA |
| | TnpB/Fanzor | FSTHKEFEQSAGTRVGGMDPGVRKYLKRRYEVIDARKSRLAKLDNAAK |
| | | AAAKDRESTKLHTDQKVANAKTVKPPKSREHRRRARLRREIQQTNTKIT |
| | | NAVIDFHHKFASWIAATFKTFLLPSFQTSLMVRKYEVEMAADGTPEKRRI |
| | | CSPTARRMLGLRHYSFSQILKHKMERAGGRLISCDCGKIKENLGGKEVY |
| | | KCDFCGAVMDLDLNGAKNIIAHEPASYAE |
| 84 | PCa-1 TnpB/ | MKRKRREDNDCPDGSDEYYPSLEFVAETPDLDYEEEKSEDVDVEAVTEA |
| | Fanzor | PAQMFPCSQRPMISSTYHLKEITKNIHSSVVCKLNRVCRHPWLVEEIKKV |
| | | CVDMKQVQLEGWHLANLHVLRCLKEGDDVPELEQMFFYRCCAATLSD |
| | | VEKRDRPKKKTGYETFHKTCQLYWSGREREPSYEPEFINYASTMINEMA |
| | | KLMQINALNMVALHFRRRLHQYIRFKYAGEGKLQLKYNETKRLVDSCF |
| | | RVKSVPEKDDSGNLTGNTIKDWTSWDDTKNPIEKELRPWLEVVPWQWQ |
| | | IRANSAHFIRKLSDMLAYMEKFVNKHPDTKGARVYSLLPVSTSYQAAYV |
| | | KLNASTLYGILSRRILDPKVKGFLDTELNHIPFDRNTFQKNRNEVLRLIFD |
| | | VEQFETRNRTFAFEVNTNGYGASVTMIRPVTTSSVVVVEKQVATKKRKK |
| | | NDETADTKKKPTKKKLKAPKELAEADTFAKELFKLGPEYSPKVLIGIDPG |
| | | MRSLVTAVSTGHLPRRRRQKSRRGKHRHHQNRRGKRKERVVQISTREY |
| | | RHLARFNDFAFYNENLKRREPWYASVIRAMPSFKTASYDVYFGHLQFLW |
| | | KHLRFLLAFSAEQSFLRWRFTRDRAKMKAMDTLAKRIVPKADKHVCIAY |
| | | GDWSRRDGLKGHATGPVKGFVKALKRRATVIPMDEYRTSITCSCCHKRL |
| | | RQSRLFNNLNRKKDEADVRLKEHPSLKEAKEIAEMAKFQNPKLANKKIV |
| | | LKCTRNVLRCTNSRCKANFWNRDVNAARNMLELLRSGLKGKHGARRL |
| | | RAFRRGL |
| 85 | PCa-2 TnpB/ | MDEKPRVKRPDRAQRIAENKTTDYVSVKTTLNSFCKRGGRFLPGRALPW |
| | Fanzor | EEVLADMNKGVLEAYLLANVHVLRLLKEGKEIPVLDHNFFRSCLSAVM |
| | | KLIVNHKIKGELGESLKVYNASRCVRSLQANGGYINQGWCHNAADQMA |
| | | TMTKNALSMNFYRRFHKFLKRTYMIDGRKAYTLLQGILSHEDYVPQGTR |
| | | FDSIVQEWREKIPRKGNGRLANDAHRLIPLTYTFLKDIEERNLYGDQVLD |
| | | EGEQYHELRVFSILPTKRGFECSHMKMCQLGLWGLLKRAGYTIPNRGSG |
| | | WEEVRDQYWRMLFNIKKFETVNRKFAGEIVTDGKAVSIIMRKPKREAEP |
| | | TKKYSEDDFDVFWGLDPGRRDMFVATNQWDETVSCSSKEFYEEARYTK |
| | | AKQKISGWRDRNPQVLEAIRNMPTKKTASLEKLKVYIAFMTQRMDFLLG |
| | | FSQLKPFRRLRLRSFIFMKKKMRKLCEQLVPRGNRVVVGFGDWSNQDV |
| | | GGIIKKCPAGPVKVFERELARHCTVIPIAEYRTSKVHFDCQHELKNQYSQ |
| | | RLCRDGEIRTRKVYSVLHCLNNGCRGMTVNRDVNASRNMLRLLQCKLR |
| | | DEDRPVAYTRQWTKKNSAIECLRVAQ |
| 86 | PCa-3 TnpB/ | MRADSSTEETEATKATRKKTQETSGGFRSRSRSRVIETVEPLLTSWFEIDN |
| | Fanzor | LAQATPTSSWRTGSRSVAHRYHEIDEVLQVQGIEDEESVNSAIKIQLHSTK |
| | | EQKKKLDQMFAAQRAIYNKMVARSLPRFMKATKSSIAGFFAALKRDEK |
| | | TSFPVLKFKSKFASTNSIKIPSRAVKVVEQDSHTAFPFHTRFFGFKEADGIA |
| | | VFEGIPTLTTSVRLHRLREGKYYLCVPRLRSFQKTESVRVCAIDPGVVNF |
| | | ATVYDPDGRTFSVTDAHKVLKKKFEAVDAMKSILALKDNACKDRHKLK |
| | | VRSKKKTGRKSPKSKEHRLRYRLRRRMRFTYRKATRCVNDLHQKLSCW |
| | | LSENYNIVLLPSFQTQEMVMRHLREVASNATPETASTEDNAAINTRKMRS |
| | | PTARALMAQAHFKFKQLLKYKMKRTGGRVVDCEEEYTSKTCSSCGVIK |
| | | TNLGGDRVFQCANCHAVFDRDVSAAKNILHKNFSLLT |
| 87 | PRa-1 TnpB/ | QRLGYPLQCLRNPKACRDLVQMILRCAKNHNSTPTLVLLCYVVHLRSVV |
| | Fanzor | AIAGTNNVFSSGNVFPTFNSPATQPTPQAQQQGQHRRHWKRKRKRENRH |
| | | GQRIVEIEYYHSAKLDKQRFWCRNLRKRETRYRELLAAIPSFKTADYDV |
| | | YVDRLQCFLQSSGFLLSFTAQHSFRKWRFLTYGMKMKALRVLAKRLVPL |
| | | RSAQVCVAFGDWSCQDGLRGNLHGPVKAFRKALERRATAVVMDEFRTS |
| | | KPCSGCHERLHQARLLTKVKDEDTKEVKFVLKGNRNVLHCKNSHCKAH |
| | | FWNRDVNAARNILELLKRKLLGLGRLAAFKRWRLGRKHDWDGD |
| 88 | PRa-2 TnpB/ | MQAVKVSRDQFFKLIKQKKRTTFPVLKFRSVNGSPNSIYLHTHNTYLKLV |
| | Fanzor | AEGEDHYVVFHPRFFRAALGKGIRISHAVQSPGHMAVSPGSAETGTTSHS |
| | | PRTWSSRRAAPRREFGTVISTDGTTFSATDQGCSKRRFDAIDALKSRLSKL |
| | | DNEAKAEVRKRHPAKIHTAQNLATNKVTMEASIKIAKSVKQRERARLRR |
| | | EIHAINTKITNATTDFHQKFTSVLAVKFKTLLLPSFQTSEMIRRYEEEMEA |
| | | DGTTLAPAQHYIDRRGRKRRIRNSTARAALSQRYFSFRMLLECKMRRAG |
| | | GRLITCEEEYISKTCSSCGKIKANLGGSEVYKCGGHGPPSLSRTSRCSFDE |
| | | VEPQRCCLHRGYLPLWTL |
| 89 | ALa-1 TnpB/ | VSGKYKGLASVYFVAAKKIRPINVSINNTVLNNIHKGIGFVEIVDEDLWQ |
| | Fanzor | YYFNTKLVTRGQSHSVEQFMVIDGMSASLIVSRPDKATVEAIKYKERDIIL |
| | | QMHETAQRIVAIDPRRSPVFTAVVKLQMNHSRNTILTISSMMLYNGLVGS |
| | | SIMNAATHTGTNMIRKVWCNSSAVITSFNAAKPSNTTGSLKLYKVYARYI |
| | | LHDLDDGLKFYSATSFKRLRWKTYIQLYMEMQTSVRQEEAIGNEPVPTR |
| | | KMRQKIGSRAKTIDFDEYGTSISSCCCHTQMEGGSHKIKQMVSDFMVCG |
| | | VVLYTSCSRKVWDRNTSAAINVLKIFLRK |
| 90 | ALa-2 TnpB/ | HGLIARIRRTRSTKREREATIECSQATQGHQDGFAECGWRGIQFSLCFPQL |
| | Fanzor | DSISERSCYHRKPDTKKDFLYYDLQCQTNDNYPAILRMTEFIEQAGSRPR |
| | | RFSPFRTSVISKHICIDTTIVVHLLYDKELRMGKILEVLTKGGIKRNESLTE |
| | | SSSDGQDFRGPDKGWNQTQRVTDRIFVGIDPNMSALLFCTSEDGRNISET |
| | | LKISDGRRYIARVRKDHTEGKKTDSNRADGLTIEQWETSLCGHNHQTVK |
| | | CDEFKAYIQAELLFNSQIETFYLSQVDLSRFWNTRKSEQWMLQRFKEML |
| | | GEPNEVDIRIGDWKQKKHRKFRACQSKGC |
| 91 | ESvi-1A, = | MESEENDSSKKPISGIKRKRAKSGSSDKAPGDDDENSHIHAKKELATWA |
| | 1ESvi-B TnpB/ | VARGIKNPQTRVCSVTSSMDACVREDVPGKDVLLRQIDKDAKSLGILRH |
| | Fanzor | LVSVCLNVLAEKDPSNTLIVNRTFIQQLFTRISGNTLHKSTRPLHHPDLQS |
| | | YLDESKLDDETLRQVEKFPLRCRDALCGEMMTSIKTHISGNFESRLVDHL |
| | | SCRLERRLWECRDDEHFWASIPRAAKNIYEAAGQKTPEQALSIIRKFVDQ |
| | | KRGSEKELLRLEWGEAMEGLLSEFRGLFEPLRVKRPPPKDPPDHPKTEEE |
| | | LTGREDFLYNVKARLNDVLQIEAGCRKVDFHLRKRRGEIWSEVNERQQS |
| | | IKSSKTKEGFLKQRHLLQEIPVPPWFDEGIEMSKKDASNLVRDVRNTRRD |
| | | MWKSMKEPGTEHPPKAFSLMPHFSLTRAFVRYDSQSIGTLAKSLDFPGVS |
| | | ENQVTLKTTGVKCAGRAERLWWTGIFDFYSERIVNRRPSTEKNQTQRTR |
| | | KVLRLNNRYRKGIGVCNKDPWFTDEVLYSAKRDDPSCETPWIVNSISTD |
| | | GLQVKVCLATLAKTNPLPKGVKELVKKGYASIQDKLRVSKSSKGVFKEA |
| | | FPLYDTEKSRLSSPDNHIEVVGLDPGQVSVYATVRANITSPETFKASDFKG |
| | | SQSSISSREYKHQSLTRFSARAEVGRRLDNVEYGQAVQAFDSVSLKQPDS |
| | | SVAYSSVIYCTLRVRVVELLSEERRLQKFARLRARQRTVDGMARDIAFG |
| | | NTYRQEVRKEQRSSAEPLSPERRKELLKKIRDKRRVVFFGNGQFGHGAR |
| | | GACPRKALIRALGVLCPVVLVDEFRTSKCCCSCGVPLKQVDGSRVFRCSS |
| | | QMDEGRSCPVRYIDRDANGSANIGVCGVFQLLGFKRPSFLCRKSSE |
| 92 | ESv-1 TnpB/ | MEVDTENPDPRAKKRCTRKSGVEQSLHAEGDEESSSTRQSCICLDLERTL |
| | Fanzor | VKSVMPKYIRDRLERMSRYTGSFRYIVGLFSNYVVIRRLEQGLQPPEVKK |
| | | VFYDRCWAAINTRISGKNTRNDFSDLLDEFIDQSGFDTSLFPPRVPCRLQE |
| | | TVTREMEVSAKNSIVVHLEAKIKGFLRFRLMNDSQLAFQHLPAKDRSRII |
| | | VSLSNDCLEREMRGDLDPGYVPHVLQVRDLLAQVYSTEPDVSVLKVLK |
| | | KKPHLFLELISMISHVAEEASDNNRDLREETKTRSDEEKVRKAFYMSERI |
| | | KRGLVDRPKMCTVLPVWKLAPCFPYYSSSVVGSIFHKEGHDFSSVSRFIS |
| | | EHFDLGRVNRKGYKPSGFRSDGYQVQVTFMALVSKKPHVPGTKDLAKS |
| | | GYQIPKKVVSLETQERGLFVLSQARKDSRKIVADRLHAYNLTVVDPGCA |
| | | SVVSVRSCPLEFCRCVGSVRERSTAWEMKGTEYSAKCGRTMLEGREKK |
| | | RRSNDEYGRCFPRFSAVKKKTARKSSFLAYCRVAAETFKVMFAEKMKR |
| | | ARKRSRFHSSRLVQKTVDKLASNIAACPSDRKSIVLFGNGSFRAKKGHAS |
| | | APRKKLVRAICSRVNVGMLDEFRTSKMCPGGCGGEMTDVQGGQRVRQC |
| | | TTVSVGVENPCPLFENGVAFRCDRDASATLNFCLAGYCGLVRKSWPTHL |
| | | LRRQ |
| 93 | ESv-2 TnpB/ | MVRPSSDGNSHKKKKHKKIPPRAPPKNIPGFLQTVASLYPNQYLTASGNL |
| | Fanzor | PKSCKGTYCCQLFSRVRFGKEVSQQLRTRTVSVGFRAATTDEKRDTFQS |
| | | KVNDAVDHLSRERVLASLFANFVFMERLQNGSALPEPDRAFFKSCLSSCI |
| | | LSRGGGSLNADFDRFSEITGLTRLVPPKSMNLDQQREHEAQRMATATST |
| | | RATHHAEKRRISITRWFLRGRISRGDLDCKKYATKLYKLAEKIITNFDGTP |
| | | EDTQKLRIMSRAIDPGTDTNIIIDFAWQEKEFSDRDRGDGKSAETIKHLN |
| | | WMFNTYVAESRRMYEEAVSRSHLFAPGKDKACRKKRDEFVKHELGEEF |
| | | TYAPPKDTAVLPICSTGAVFIYIDMKTLGSWGLLHSNDAWWYKDVLRPF |
| | | SKEANVKCLVNKENARYASSEHGYLSSLLESGPDKCPWMIGESFLTDGV |
| | | QIKLLLKTLEHGRKAFSGSSELNEEGYNKLPRADVQLESLLGEGRGVYNI |
| | | SSLLKSTSIREDVVVMSADPGQAKVINVSSATARTWAKQDPGVILKTSTC |
| | | VFGDDYRRDTLASRSDEYETFRRKGQPYGVAIDRLGNEKKRTSCLATFIQ |
| | | YCRRWCVNGPVLFRETLQTGRKFMRFDRFRATQKTLALIADKYMGHVE |
| | | DTCRVMLFGKASFRAQKGRASAPRKALIREMASRGVVLMVNEYNTSKM |
| | | CPGCGEDMVEDKERRIRSCKNFKIGSPEESCRLHRLTPEFEMDRDDVGST |
| | | NIGMRGFGKLLGQVWF |
| 94 | SWv-1 TnpB/ | MSASLLTETSLEEEQRREVNNNTSKENDTMEKKTETAATTEKDPEPSVSK |
| | Fanzor | RSRNKEPKTTSTVYTSVKCYLSSIIKSESSRSNVTSTKERFEERCKSVSKM |
| | | MVKGSLFLRLVVDECLRRYNHLEDEIDKWPDMTKDNFYVQLLRKGLDK |
| | | KKLKEGSTHPVVEDVWNSPIVQETFLSQQGEGNNPIKRHLMDENTITYAA |
| | | KQLKTCFETNLRTHFRTRQQRAISGWLAENGEDKKYTKLVQHWIIGCTY |
| | | KSDWVDSGDLERVKEGTKNFVTLHRKHLCVISDKKNGTISYSPEEKYPIP |
| | | SILNYYKFLQTEYPQNKKIQKMIVVPKHKLKIHYCTFDQTTIQGICKDLGV |
| | | WKDMEERHKQSEDILYKQGWYLLFDVKKIKKLRPNWNFHSIQTDGEGV |
| | | SVLFSREVEEVETVSKKSKKNKKPRGDEDRRNYPPTNAKYVVGVDPGRT |
| | | NVVSCSVFDTRQKRVVRKHRMTAKQYYQESWMTDRRKANETYKKNN |
| | | KEYKEALEEITRYDNGEEIINDGNGDTSTPTKKFEAYLKVVNEHYRLLW |
| | | NEKGKKKYRKNAMKVYSRKQKCISNFIDELIPKRDKIEDYHIAFGDAKFA |
| | | CTGRGEQYASPARIFAKKIKERVGGDKRFTFVDEKYTSKVCHRCNQPLN |
| | | MLEKDCFSPNKKRKPPTIVTTTTTTTTEEDEENGKWKKATPLRENRDTRR |
| | | CSSEKTQFGYSSNRKVSTGDISMETPVPSSTSSSFCTPTSITCVLGGKFVDR |
| | | DFNASTNIVHKFLGFWDKKLMEKKDKMPLKYHFIRVA |
| 95 | HAgv-1 TnpB/ | MPVCKSCGGLDHQRRSSTKCANFLPQVRKRKLEPQQVNLDLQVYVVKR |
| | Fanzor | GLQSALCPLLSDDDRNRLLYEIRRDVRDLSRVYIELGILFNYYMKRCDTL |
| | | PDKPDVLNFFYAMKGKGPYAACYKHLAGDQVYDGKLRSFVVQELAKQ |
| | | YETLIHTNIETHAYSRLARYFGVKKNDHGLFLAYYKKDFSQATEDMIRV |
| | | CQIINVDSCKKNWWSTVPLWLKIQQDLFDRDNVESFRLFPQPSYGLKHLT |
| | | YTSRGWHELLRRVKPDSVPSHWPNIQDHIKELWAPWLKSTKNFGCCLQT |
| | | DGVSVSLSMNRWIKKVTKNGITKEVVIKKWKPSYDKLIAVDPGSRVPVA |
| | | AFNSHGFYKRVTSKFVKSHSLDWKRERVRTKLTKSSETAEAKDRERVQT |
| | | TCNIVLSSKNTSHVVEYTVFRLKWFDVRQKPYETHSKLTRLMFDKYINT |
| | | AKLQEKIVKEVFDGGGVEKTLVLYGAGSKFMNAAQFSGRKFKHDALLK |
| | | KLRQKRNIHVEIVDESYTSQACSTCNQQTGEFSKIVMDHKKRVGVCPNC |
| | | ASRFDRDQNAAKNILINFMRSTVCRPRCSYTKRVS |
| 96 | HAmv-1 TnpB/ | MPQSCKSCGGEGHSRRTSKSCANYLPPIRQRINWDGISTEEFDVGVVKRG |
| | Fanzor | LNGVLCPLLDEEVRRRLTAEIRSDVIELSRLYVLLGVFVNHCWNAHPADE |
| | (ACH88588.1) | SNSVRTFQDMMNYVYALKGKGPHAQKFDEMVRYHNGITYYNGRLRNY |
| | | AVQEMAKTYWTVLKTNITTHAYSRLARYFRVKRNDPTLFAAYYHKEFG |
| | | DDDISIVCRFINVSSDWHSTIPMWIEIQREMYARGEQSFVIFPQPSHGLKH |
| | | VTYTSKGWHELLRRVTPLTITSNWPSITDHKKELWAPYLDTNVVDMKKF |
| | | GCCIQTDGVAVSLSMNRPKRKPTSTLPKSYVTNTFNEDRIVAVDPGSRVP |
| | | VAACDSRTGFKRITKRWVRSHTLEWKRERYRSQKLRHVELNEVADRHR |
| | | VELDRGVQITCRNGHQVKLYTDFRLKWFDARQRPFERTRKLTRLSFDKY |
| | | MMTARTNERIVRETFLNDTPSDKVLVLYGAGVNFVNVACYSGRKFKHT |
| | | DLLRRLRSRRNIRVRLVDESYTSKACSDCNELRNGTYTRLRMNHRLRRG |
| | | VCPDCNADVERDYNAAKNILVNFQRSAVCRPSPSCDSGVTTDKDSHSIRP |
| | | IIQ |
| 97 | PUgv-1 TnpB/ | MPLCKSCGGLDHQRRTSTKCANYLPQVRKRKLEPHQENLELQIYVVKRG |
| | Fanzor | LQSALCPLLSDEDRNKLLYEIRRDVRDLSRVYIELGIVMNYYMKRCDVLP |
| | | DKPDVLNFFYAMKGKGPYAACYKHLAGDRVYDGKLRSFVVQELAKQY |
| | | ETLIHTNIETHAYSRLAKYFGVKKNNDGLFQAYYKKDFSHATEEMKQIC |
| | | QVINAESCKKDWWNTIPLWLKIQQELFDRDKVESFRLFPQPSYGLKHLTY |
| | | TSRGWHELLRRVKPDTTPSSWMSIDDHIKELWEPWLKSTKNFGCCLQTD |
| | | GISVSLSMNRWVKKVTKNGETKEVVIKKWKPSYDKLIAVDPGSRIPVAA |
| | | YNSNGVYQRITSKYVKSHSLEWKRDRVRNKLTKSSEMAEAKDRERVQA |
| | | ACSIVLSSKNTSHVVEYTVFRLKWFNVRQQPYEIHSKLTRLKFDKYINTG |
| | | KVQEKIVKQVFDGGGVEKTLVLYGAGSNFMNTAQFSGRKFKHNALLKK |
| | | LRQKRNIHVEIVDESYTSQACSACNHKSGVFSKISMDHKRRRGVCPNCSS |
| | | SFDRDQNAAKNILINYLRSAVCRPRCSIRRVS |
| 98 | SFav-1 TnpB/ | MPLSCKSCGGEGHTRRSSKSCANYLPPIRQRRNSGDVDHYTQDFDVGVV |
| | Fanzor | KQGLDGTICPLLREDVRQRLKVEIRSDVVELSKLYILLGVFVNHCWNAYP |
| | | DDESNSVRTFQDMMNYVYAIRGKGPHAKKFEDMVRYHGNIRRYDGRLR |
| | | SFIVQEVAKTYWTVLKTNITTHAYSRLARYLGVKRNDPTLFAAYYHKEF |
| | | GDDDSISRVCRFINVSSDWHSTIPMWIEIQRDMYARGEQSFVIFPQPSHGL |
| | | KHVTYTSKGWHELLRRVDPSNVKSNWPSITDHKKELWAPYLDTCVVDM |
| | | KKFGCCIQTDGVAVSISMNRPKRTSVRRKAALPKSYTIDAFFEDRIVAVD |
| | | PGSRVPVAACDSRTGFKRITKRWVRSHTLEWKRDRYRSRKLRRVELNEA |
| | | ADRQRVELNRGVQITCRNGEQVKLYTDFRLKWFDARQLPFERTRKLTRL |
| | | SFNKYMMTARMNERIVRETFLSDTPSDRVLVLYGVGVNFVNVACYSGR |
| | | KFKHTDLLRRLRAKRNIRVQLVDESYTSKACSECNELRNGTYTRLRMNH |
| | | RLRRGVCPDCNADVERDYNAAKNILVNYQRSAVCRPSPSCDSGVATDTD |
| | | PHSIRSIFQCAVSEPGS |
| 99 | HVav-1 TnpB/ | MPPSCKSCGGEGHSRRSSKSCLNYLPSIRQRSNCDDVSTEDFDVGVVKRG |
| | Fanzor | LNGVLCPLLDEDVRRRLTAEIRSDVVELSRLYVLLGVFVNHCWNAYPNN |
| | | ESNGVRTFQDMMNYVYALKGKGPHAHKFDDMVRYHDGITYYNGRLRN |
| | | YSVQEMAKTYWTVLKTNITTHAYSRLARYFGVKRNDPTLFAAYYHKEF |
| | | GDDDISRVCRFINVSSDWHSTIPMWIEIQREMYARGEQSFVIFPQPSHGLK |
| | | HVTYTSRGWHELLRRVTPSNITSNWPSITDHKKELWAPYLDTSVVDMKK |
| | | FGCCIQTDGVAVSLSMNRPKRKPALRKKPISTLPKSYVTDTFNEDRIVAV |
| | | DPGSRVPVAACDSHTGFKRITKRWVRSHTLEWKRERYRSRKLRRVELDE |
| | | AEDRHRVELDMGVQITCRNGHRVKLYTDFRLKWFDARQRPFERTRKLT |
| | | RLSFDKYVMTARTNERIVRETFLGDTPSDRVLVLYGAGVNFVNVACYSG |
| | | RKFKHTDLLRRLRSRRNIRVRLVDESYTSKACSDCNELRNGTYTRLRMN |
| | | HRLRRGVCPDCNADVERDYNAAKNILVNFQRSAVCRPSPSCDSGVTTER |
| | | DSHSMTIRIIFQCAVSELGS |
| 100 | MCnv-1 TnpB/ | VIFPQPSHGLKHVTYTSKGWHELLRRVTPLTITSSWPSITDHKKELWAPY |
| | Fanzor | LDTSVVDMKKFGCCIQTDGVAVSLSMNRPKRKQTSTLPKSYVTNTFNED |
| | | RIVAVDPRSRVPVAACDSRTGFKRITKRWVRSHTLEWKRERYRSQKLRH |
| | | VELNEVADRHRVELDRGVQITCRNGHQVKLYTDFRLKWFDARQRPFER |
| | | TRKLTRLSFNKYMMTARTNERIVRETFLNDTPSDKVLVLYGAGVNFVNV |
| | | ACYSGRKFKHTDLLRRLRSRRNIRVRLVDESYTSKACSDCNELRNGTYT |
| | | RLRMNHRLRRGVCPDCNADVERDYNAAKNILVNFQRSAVCRPSPSCDSG |
| | | VTTDKDSHSIRPIIQ |
| 101 | PGv-1 TnpB/ | MKKKKKGEFQEFRNNEKSAYKTFKIPIKSILYNYDTTQPVINNLVFEINDL |
| | Fanzor | MIHAYQFIRLYVLSCYTNKQTLPIIDDIFILYCIKTLGTRDNRGKKCKDTA |
| | | LLDTLEKFYLEEYQPLLNHEKTNLKNTTFLLPYLATQIHTSLSNNAQEHFI |
| | | QHFLRFINTTTTAITEDKSILFKLKHQLMSLDNETNEMFNEWKITHLHNIF |
| | | PQNIKKSIHYDVKVRPFEYLKGMLYMNEVLEKEEHKLFQPLPLRTNIIPK |
| | | HIILDTASLVSLFCPETDKDGNKNKKGNLLSNIKDNQYDIWNSFLNLNHK |
| | | IFRNQHYQFHHQIQTDGISCCLLFIRKDLKDKKWGSRVPSIPEQDFYNIED |
| | | LSKEQLDTLKDRNIVGCDPGKRSLVYMMDKNGNKLQYTAPQRKRESKA |
| | | KTNQRILLEEKKRHKIIEKETELSLQNSKSVDYIKFKTYLVEKHKLNKETT |
| | | EFYKKETWRKMNFRQYSYGKKSIDTFLNKIQETFGSNILIGYGNWSRSTQ |
| | | MKHFMPTMNKGLRKQIHKKYDTITINECNTSKKCCDCNKDLEYYKDKE |
| | | NKKVFRLLVCSNCVSCENKKIVFRTRDANSSINIMKLTSCWIDSQTRPTAF |
| | | HSHISSFTCSTIKEEQEKVRPS |
| 102 | EHv88-1 TnpB/ | MPRNKKDDARVIKCPLDKILKPEIVSIIRDIVPVYNRIAISATLFLNLRIRQL |
| | Fanzor | VESRASSTEFAQFFQANKLVKYFMAVTSSTLGAGDLHAAFGTNFPSVIRE |
| | (AEP15317.1) | DRPKAFSQVCMYICRNLEATATTNVWYHMKKRVCTVVKKTFGIPRDEW |
| | | LHMSNEEKKNHKIHLKQVNVDVLSLEGDAFVSPEQYHDWISNLRSEMK |
| | | VTHVTRQLLADTRGVDVDDPNVPGMVYYMKVKPYEFIYAIATLSSHLNS |
| | | ITGGGYALYPIRSSMVPKYLHLDELILADISPGISKGATNTAAGKKRTADG |
| | | SELSNKDKLVARRMIVFKHVMTPKAFNKWENHLGWSVDTDGYGCSLH |
| | | VKKKVNELPPSTHPSIHNGLIKIDAFKDKFSERYSINDLSIIGVDPGKREIVS |
| | | AVDSMDYTSKYTFRKTHQDHLTGTVRHRKAMECTKPQSVFETEQDLAT |
| | | TSKKVVPVERFMDYATKLATSISSSSGFYEQGVYRKRKWKRHLRLQKAY |
| | | SGIVNNLKMVSCHPNKPIVLAYGSWGMTTSGLPFKGLPPTIGIGMVRYLA |
| | | RFFPVVITPEYYTSTICPCCDSNVTRCTHVEQQRASPFFENGRLKEIRGLK |
| | | HCPGCNTYFNRDNMGAWNIARNFTNMYQGMAPIRVRNAAQAAEQELT |
| | | NRTEVD |
| 103 | EHv99B1-1 | MDDSHPCLIDMLNQQQSNNIMESTAEPLINGQEMEQLNTCDQHQTDNGY |
| | TnpB/Fanzor | MGSSMETQVMDMFPSSMDESLPESNSITSDDSSMSSNRSTQDALPSPIVLP |
| | (UKZ11142.1) | DLTTTAPVLNRFYDSRLKDVYNMFIYPTETDSVGSATISSSTYSTTMEQQ |
| | | LKLTNKMNSLPQTNRPSLQTTSFLSSQFSVLGCTEREVEAAVDAQVGEKR |
| | | KKIPFQRQTELQGNLRTYKIKMFPTPEQKDELKRTFGMARYAYNWAIAQ |
| | | MKEGKKPNNIQLRNEFRKLPLPQWASEKKKQVATRITAGAIKQAVDAVL |
| | | TNLKKKKKDPSHHFEMTFRSKFANSEVVNIEKAANGGSLAKMQAYTSE |
| | | HLPYRAGRKNALLYFGNNLKQAGPILIEDNPNVIDMFVAEGKPKEDGKIL |
| | | YDRRRNTYYYIYLYEQPALRDPDPGFSDKDIVCTDPGEVVFQTFYSPSGS |
| | | HGELLLDFRQVLLERCKKLDILHSRVTKKEKMRRQGLLQGRKNYLIIRRL |
| | | KQAWARERARLSAFVERAHYFAANYILKNYDIIIQPQMAVSKMVSDLNS |
| | | KVARSMLTMSHYMYNRRLEWASTRYAGRYIVHANEPGTSKTCTNCGY |
| | | WHADLGTSRTFKCPSCGIAVSRDLAGARNNCLHAVGTVIRNRQLSEKR |
| 104 | Acanthamoeba | MKEAVKNVKPKVPAKKRIITGSKTKKKVFVKKKPPDKKPLKKPVKKTV |
| | polyphaga | KTYKLKSIYVSNKDLKMSKWIPTPKKEFTEIETNSWYEHRKFENPNGSPI |
| | mimivirus | QSYNKIVPVVPPESIKQQNLANKRKKTNRPIVFISSEKIRIYPTKEQQKILQ |
| | ISvMimi_1 | TWFRLFACMYNSSIDYINSKKVVLESGRINVAATRKVCNKISVRKALKTI |
| | (APmv-2) TnpB/ | RDNLIKSTNPSIMTHIMDEAIGLACSNYKTCLTNYIEGQIKKFDIKPWSISK |
| | Fanzor | RRKIIVIEPGYFKGNSFCPTVFPKMKSSKPLIMIDKTVTLQYDSDTRKYILF |
| | | VPRVTPKYSVNKEKNSCGIDPGLRDFLTVYSENETQSICPIEIVVNTTKNE |
| | | YKKIDKINEIIKTKPNLNSKRKKKLNRGLRKYHRRVTNKMKDMHYKVSH |
| | | ELVNTFDKICIGKLNVKSILSKANTVLKSALKRKLATLSFYRFTQRLTHM |
| | | GYKYGTEVVNVNEYLTTKTCSNCGKIKDLGASKIYECESCGMYADRDE |
| | | NAAKNILKVGLKPWYKQK |
| 105 | ISvMimi_2 | MAKTTNGKFWNDECEKVSDLLWKPNLIVNHKPQERINFKCDNKYITFNY |
| | (APmv-1) TnpB/ | YSNNVPINDQLKFKDIPQSNNTDKKNKLMDKLLKIEDSRYKKDIESNKTR |
| | Fanzor | KIKLTKEQLTAKHNTQLLKIQNKISQLDGFIRSRRIQLLPNDFQKNIFNIWF |
| | (YP_003986594.1) | NETTVIYNRLLTEFTQYYIKFKEIASDNKELVKLLRTNEEFPLSLQKLRNL |
| | | KIHDIINIYLEDQLIHKLETPFCVIANTIKEFVANVKGNLTKLAENKIDKFT |
| | | FKHKNFNGKIRTISIDSKYASENSFYPDKLGKLLQTYEDLKGFSWKNVKH |
| | | DFKIVYDKYNNKYYAHIPKYVYRNLNILNKKPITVMDPGERTFQTLYGL |
| | | DHVISIGDNLRETISKRLLRIDNLKSKLDTPGSKKHNLKLNRKTRVRKCR |
| | | YKKAIDRHHKKIEHLQRELHYKTAIYLCENYQRIMVTDFSSKKVSSKDG |
| | | DLDAMSKRILGKISHYRFRQRLQQKCEEFNCQYLEVNEAWTSKTCCNCG |
| | | YIHAKLGTSKIYDCPKCKQKRDRDVNGAINILLKNRKLVLMEDQ |
| 106 | APmv-3 TnpB/ | MVYNQDLSISNKTCGISDWLPPVRNKKISDIKSNSWFDIRRCLNPNIKTKF |
| | Fanzor | VPVNNKQIVLKEPNCIYLKYDLITLNPSSDHIRILREWFSLYAKIYNYAINL |
| | | IRKMFYVNNKLIKSRLQCEIYDEIIIRKLSNDKTKLIESLKLSDRPPEFLLDY |
| | | AISHACTYCNSQVKKYLKDSKNPYNKKTLRFRLWSLHKSRRVMHILPPK |
| | | LCDKNVCNGLFLKLNPSKNIHDIYMFCVMSWDRLTGKFFVYKPVLKRLI |
| | | NIPKKQFTRCGIDPGVRTFLTLYSSERTYDVCDSTFYNNKIKRMWRKIDRI |
| | | NFLLHNRKKRKKRKNDRRLRKSLAKYYRKIRQRIKDLHTKTAKYLVSRY |
| | | KEIYIGIYDTSKNLSDPRISFYEKRRIATLNHELFLTKLQQVADRFGSSVFA |
| | | INEHMTTKTCSKCGRINEIGSSKVHKCACGMYAGRDENAAKNILKKGIK |
| | | EHYLSAQSDKNVTEYNNKKSNNTNKET |
| 107 | MGvc-1 TnpB/ | MFWNNQCAKLSKSLWIPSNIINLKSNKKKYFKCDNPNISFNYFTDGIKINK |
| | Fanzor | KIKFNDIKVTDNHDKKEKLRNKLIKTENTRYIKEQNNQKCKLNKQELND |
| | | KHKKFLDKIEIKVNQLDGFIRSRRIQILPNKKQQNIIQKWIYDTTSIYNKLV |
| | | SSFNQIYDKCQKIVDNMNVENKFYHLGKIIKQNTEFPINFHKLRALKCSDF |
| | | IQDYDRIPYCVIADIIKEFVSNIKSCITKISKGLITEFKFKPRKYNRIFSYIPLE |
| | | SHYTTVRGFYPSIFGKIKTNEKIFSWLDIKHDYKLIYDKYSNKYYIHVPKY |
| | | VYRNDLTIKKPIAIMDPGARTFQTIYGLDHVVTIGDDLGSTFKKRLLKIDN |
| | | LKSKLSKPGKYKHNKKLDRKTKVKKWKYKRAIDRHHKKMDHIQQELH |
| | | YKTANYLCSHYDRVVVTNFSSKKVGSKKNDLDAITKRILGKLSHYRFRQ |
| | | RLLTKCEEYGSQYYEVDESFTSKTCCKCGNIHKTLGSSKIYDCIKCKNKID |
| | | RDVNASICILIKNKDILLK |
| 108 | MGvc-2 TnpB/ | MVYIKNRIPIHYPNKNCGLPNWVPSINNYYDCLNTNSWFDMNICKNPNK |
| | Fanzor | SSVKRHLIDAPIVPNESYKQQMKSIKRCKSNKEPVFIYTRKIRIYPTDDQK |
| | | CILEEWFWAATRMYNITVKYIRDFLFKDHKLTCPFASKDILINFPFRTVLK |
| | | EDRDKLMKSMKNPIVGHLLDEIIAQVVSNYKAAISNILVGNIKKFKISTW |
| | | KYIREKYILKIEKNLFSNGTFCSRTFKELKSSEPLINIQHTCTLQYNRDIGK |
| | | YILLVPTHKETKSYVINDLDCGIDLGVRTFATVYSRNNVVTIGNKFKKID |
| | | SCHKKIDKINELLDSDNNKCKLRKAKRKYYRRIKDIITDMHYKSAYYLVT |
| | | NYDNIHIGKLSTKGILSKNNKKISRHTKRMVGVLSPYLFRQRLNHMAHK |
| | | YGATVFEVNEYLTTKTCSNCGKINELEAKKVHKCKCGMKTDRDVNAAK |
| | | NILKVGYQYNSTLHQVNDIDIIETDNHIVTENKVIKEYEVFEV |
| 109 | ISvAR158_1 | MVLKRWCGLYRRMYNLAIEKMLDTGRWHWKGHKVLTTYDNIPKNEK |
| | TnpB/Fanzor | VRLLPTNIRKEAYREASTAMAKAPHKNTVKFKTLKRNGRSLRVFGAVSI |
| | | VDTPDGKRIKLTDSQKHKQLSKELIGFDQKNLADIRIGSSSQIVYDGKWW |
| | | LLSPFEVERSNVPKKHAVALDPGVRTFQTTYSPDGTSKKLGDQASCRIFR |
| | | LMKYIDKLAVFKDKRTRGKRLRLERRVGNLVDEMHWKIARELATDYDT |
| | | IIIPPFETQKMAKKWKNGRYRKINKTAVRMMLRLAHYRFRERLIHVANK |
| | | LGTRVVICTEEYTSKTCGNCGVVNADLGSAKKFKCPRCHIHVDRDGNGA |
| | | RNILIKSVCC |
| 110 | ISvNY2A_1 | MRCHEISWPSGKLDQHLRNKFSIGKHNDIEQYISIHGNNIEPEQIIETTKMN |
| | TnpB/Fanzor | TETRKKEPSRRYINPFFLGKLWLSECPKDFRARATFKAADAYKTSLTLWR |
| | | KGLIRSFEMKFSTKRRELDRGYCFGIEKKVKFEDTGLKKRDGKLIITGIDG |
| | | DIRFFEKPPINKEPVAGCELSKDSCGDYYLHVPIFRKKKERKGDNFVAIDP |
| | | GGVVPWAFYASKGESGCLGTEMNERMSNVSKKISTIDKKLSGQPKGDK |
| | | NSNDVIERRKLFRKKKRIRDHEHYRVINFLTDRYDGIILPKLKTKNISKAL |
| | | KSKANHDMFDISHYTFLRRVGERCIESDTILEHPGEQWTSKTCGRCGKSN |
| | | LPGRERRGTFREYKCLYCGLHAHRDVHAARNIFMKWYIELKK |
| 111 | ISvNY2A_2 | MTLKPWWSNKSQQLTKIISWNPTNTNIFQNDTTIKKVLRTRKVKLNPTK |
| | TnpB/Fanzor | QQKMMLMKFADAARYSYNIAIAAVNDKTHKANKIELQNAFSSLKRRDD |
| | | DSYNQFFQKRRWLLATPQPIRKQAIFEAAKNFKAAFTKLKNKNIDHFKM |
| | | KFKTKKHQRQHGFSLGIGSHLKHDNGVLTILPRSLGQMRYFGFLPFEGKP |
| | | DAECRIQRDPYGDHWLLVPVYKTTKTQISKPIVAIDPGVRTPFACFRSDG |
| | | DSKCIGEDMNAKLIDIKTRVTIVDRRISKCQDGSLKRKLREHRRVLFRKH |
| | | QRVRDAFHWKIINDITDECSGVLLPPFETQRVSRMLKAKTNCSMLGISHY |
| | | TFRMRMKGKCEEKGLLYDEPTEEYTSKTCGVCGLINYLLGSKKTFECFC |
| | | GNVCDRDVHAARNILLKWLSSEAGARVAETFLASRSS |
| 112 | CRv-1 TnpB/ | MNIKNYHWFKNKKRKNINTDGWFNLKSINNKGFNDYDNIEKPINKFLCC |
| | Fanzor | KTIEIYPNLEQKNIILKWMDLFINMYNHTNYFINNNIYDFTNRKIKSNVKEI |
| | | LNFRKLRDNYIKNKKNETCKDEINKHMLDEAINHCVSKHKSCITNYFSKN |
| | | IKFFRIRNMKKNRRKKILIIEKGLFSKKKNGFCLTKLQEIKSSESIGVNKTSI |
| | | LQYDKHLNKFTLFIPIEKNTTNIENRTKCGIDPGIRTFLTCYSKKEILEIGEN |
| | | CYEKYKNNYKKIDKTRELFDKKEINKKQFTKSINHNFDKIRNLTKDLHFK |
| | | VSSYLCKKFNIITIGKISTSSIVLNTNNLNKISKRCMLSLSHFKFREILEYQC |
| | | NKYNTQLNIVDESYTTKTCSNCKELNNIGSSKKYQCKSCNFKCDRDINAS |
| | | INIYLK |
| 113 | FEsv-1 | MKDKGVVLRCRKVRILPSEEQREILKSCFGIHRHIYNECVKAEKDGDIDG |
| | (YP_002154624.1) | AGVRECYKWRKAFTTREDYYGQGKNWKDDCPSHTKQQAVEEFFKNKK |
| | TnpB/Fanzor | TGLKLVSEGKIRRFDIGFKSRFKCRQETIPFEKFLIKKKDSTNPLSKSLVST |
| | | TYKGKSMDLRIMGKVPKVFKDRDDQGFTRTEMKITRTKTGKYYAIVSFE |
| | | VPQTSRFPEIHGDMISFDPGCKTFQTYHSPDGTWGEIGTFEKQELLLRKAD |
| | | RLKSRLELEGKDKDSRWRRRTRRRMLNLFEKVRNRTADLHNKVCSWVV |
| | | NTYRLVLVPEFKTSQMVSSKKLYSKTCRKMMTWSHYRFRSKLIAMAQK |
| | | FTDVKVRLCNEAYTTKQCGSCGVINRSMTLNDRSFNCVSCGLSSSRDGH |
| | | AARNVGLRSLRFLV |
| 114 | PGv-1-2p TnpB/ | MRWIYILKCEDNYFYVGETARLYRRFWEHTAGIGGLNTQIFKPEEIVAIY |
| | Fanzor | KVDTIGKFLEYNNNIIDKQANNLIDYNRWLITHFNDCAKYKDYFDKLEA |
| | | ENNIAECMMIHNKDNWEKIRGGKYTKFNCDYKFPINNNIKELPMCKCGV |
| | | PCDIKKNDDKNYLFFRCAKKNMWENFKDDFDIEDEPCNFFMEYITDIEFR |
| | | IDEKKRVANYKQNFVDLIKKSFWLNNVECEIDDECPECVSCNKYVWCDK |
| | | NGEFKNNGIEYGNERRLLCRDCFVNKNEELSQKYSLTGKCLIKL |
| 115 | Mariner-1_ | MTTEIKFNNQYVLSRMEENKLIKIKLIIQELKQIAPLLSDVELLQLYNKSISI |
| | OLpv-2p | HQGKIQGNGNFLENDILVGMLELNNIPYKKQVTINKSGIIVGFNEKKGKC |
| | TnpB/Fanzor | YHIIDFVIGENIEVGKSITEYKVISCKTTCRERWTQDDWSYTFIPKLYILLTI |
| | (ADX06147.1) | SDDYPPSTRFREDIQRKIITCIPKRKDDRIYKLKFENLIEQL |
| 116 | Mariner-1_ | MVKYSCEKCGKEFTQKGHYTKHTTKKIPCVFESKIEEMIEKVLAKKINEI |
| | OLpv-3p | KTTEVILNEEIPENIIENIKFIDLFCGIGSFHYSFKKLGWDCVMSCDIDKAV |
| | TnpB/Fanzor | KETYKSNYGILPLGDITEIEPKNITNYDILCAGFPCQPFSQCGQHKGFDDK |
| | (ADX06148.1) | RGTLFFNIMKFVDYHKPKVIILENVMGLLNHDGGKTFEKIKCDIETSNYSI |
| | | TYKVIKCSDYGLPQMRKRLIIVGVRNDTALINHIDKLLDLDEYKKETTLT |
| | | ELLGKNFEKKIAYTIRCGGKNSPIDDKHNWDGYMVDGKEYRLTKEDCL |
| | | KIQGFSSDFKLCGNNKDQWKQLGNTIPTIFTEIIGLNLKKYL |
| 117 | Mariner-2 PGV- | MNNSKMDLQIEAITTLLSSLAINETKIIKIQKWFRGCILRLKQLPLIMYKIK |
| | 2p TnpB/Fanzor | KYLKLQAFQFSTQNEDGRINSCIDEDEVIKLLIEKFGGKIKKPKIRMWYDI |
| | | LAFDYMYGWVPINIKTTTTITSDNTGNLAMCVYAYTNEILDINRDKSYEN |
| | | GKMSDILFNKLKNKKYNTNNKKDYYFIVLNKTDASDIIVNSVKGLTILTP |
| | | NINNLPFQVCWDKNRKFKYENINKKIKLFIDCLQKPKPSWKETFMSNIRT |
| | | LDL |
| 118 | Mariner-2 PGV-3p | MVFYSCEKCGKQFSQKGHYNKHINKKLPCVNETKLKEIIGVVVNEKLNEI |
| | TnpB/Fanzor | NNNKEKIDLFISKDLEETINTEIPTMTTKHNLGQYFTTHNELKEKVFEFILN |
| | | TPSNILEPSMGQGDLITFITDKIPSITFDMYEIDKKIKLLDKIQKDKINYGDF |
| | | MTQTITKTYKTIVGNPPYVRTKKGNLYIDFTEKCYNLLEDNGELIFIVPSD |
| | | FLKLTSASKLLNVMMTNGTFTHIFHNHNEKMFDNASIDVIVFRYCKNSLI |
| | | DKKVLYNDKLLYITNSNGLITFGEEENNNSVLFQEYFDIYVGLVSGKEEV |
| | | YKNEELGNIEVLNGEDKIDKYIYIENYPCDNEKINKHLSHHKKELIERGIR |
| | | KFNENNWFEWGAPRNITTINTNLGKDCIYIYNLTRKPNVSFLGKVNYFGG |
| | | GLIMLKPKKKCNLNNIVSYINSNTFKDNFMFSGRFKIGHRQICNSYIPSEY |
| | | L |
| 119 | Deinococcus | MKRLQAFKFQLRPGGQQECEMRRFAGACRFVFNRALARQNENHEAGNK |
| | radiodurans tnpB | YIPYGKMASWLVEWKNATETQWLKDSPSQPLQQSLKDLERAYKNFFRK |
| | | RAAFPRFKKRGQNDAFRYPQGVKLDQENSRIFLPKLGWMRYRNSRQVT |
| | | GVVKNVTVSQSCGKWYISIQTESEVSTPVHPSASMVGLDAGVAKLATLS |
| | | DGTVFEPVNSFQKNQKKLARLQRQLSRKVKFSNNWQKQKRKIQRLHSCI |
| | | ANIRRDYLHKVTTAVSKNHAMIVIEDLKVSNMSKSAAGTVSQPGRNVRA |
| | | KSGLNRSILDQGWYEMRRQLAYKQLWRGGQVLAVPPAYTSQRCAYCG |
| | | HTAKENRLSQSKFRCQVCGYTANADVNGARNILAAGHAVLACGEMVQS |
| | | GRPLKQEPTEMIQATA |
| 120 | Staphylococcus | MNASSKRKIISQSEISKKIAVMNEEMQGFWANNSWDIRKCPHPSAIELSK |
| | aureus tnpB1 | NPALRNRWVRFERVKNLWLRTELKYFYFYHLNNGIWNAKTVWIRKGTV |
| | | INKMLDFLDLKYPSITSITEVPIEKAMTEYRTYLTKRGVRITTTNYKITAN |
| | | QEKTPVKANSYYVTNLKQFMEFYENFYFDGEEWDKDVWDRRNLPLPDD |
| | | KVNPTQYEYTINFKGFRNTYFKQLVKRYCKLRLNVDSFSYVSDIAQRLKE |
| | | FFNFLDMKFKQVQRVHQLTRVEIEAYLSELNMMGIKPSTITGRISILEGLF |
| | | STLLRLEWDDVPSKILIYSEDYPKIPRAKPRFIDEFVLEQLNSHLDKLPEYI |
| | | ATMTMIVQECGMRISELCTLKKGCLLEDKDGDFFLKYYQWKMKKEHIV |
| | | PISKEVALLIKVREDKVSEEFPDSEYLFPRKDGSPLKQETFRGELNKLAYE |
| | | QNIVDKSGEIYRFHAHAFRHTVGTRMINNGMPQHIVQKFLGHESPEMTSR |
| | | YAHIFDETLKNEFTKFQEKLVTNNGDVLDLDEDNEVDDVELQWFKKNIN |
| | | AQVLPNGYCRLPVVAGGCPHANACLDCTHFCTSKQFLPQHEEQLERTEE |
| | | LLAIAKDKQWQRQVETNSRVKERLEQIIGSLTG |
| 121 | Escherichia | MKRLQAFKFQLRPGGQQECEMRRFAGACRFVFNRALARQNENHEAGNK |
| | coli insQ | YIPYGKMASWLVEWKNATETQWLKDSPSQPLQQSLKDLERAYKNFFRK |
| | | RAAFPRFKKRGQNDAFRYPQGVKLDQENSRIFLPKLGWMRYRNSRQVT |
| | | GVVKNVTVSQSCGKWYISIQTESEVSTPVHPSASMVGLDAGVAKLATLS |
| | | DGTVFEPVNSFQKNQKKLARLQRQLSRKVKFSNNWQKQKRKIQRLHSCI |
| | | ANIRRDYLHKVTTAVSKNHAMIVIEDLKVSNMSKSAAGTVSQPGRNVRA |
| | | KSGLNRSILDQGWYEMRRQLAYKQLWRGGQVLAVPPAYTSQRCAYCG |
| | | HTAKENRLSQSKFRCQVCGYTANADVNGARNILAAGHAVLACGEMVQS |
| | | GRPLKQEPTEMIQATA |
| 122 | Acanthamoeba | MKEAVKNVKPKVPAKKRIITGSKTKKKVFVKKKPPDKKPLKKPVKKTV |
| | polyphaga | KTDKPKSIYVPNKDLKISKWIPTPKKEFTEIETNSWYEHRKFENPNKSPVQ |
| | mimivirus | TYNKIVPVVPPESIKQQNLANKRKKTNRPIVFISSEKIRIYPTKDQQKILQT |
| | MIMI L79 | WFRLFAYMYNCTIDYINSKKVVLESGRINVAATRKVCNKISVRKAQKTIR |
| | | DNLIQSTNPSIMTHIIDEAIGLACSNYKTCLTNYIERHIKKFDIKPWNMSKR |
| | | KKIIIIEANFFKKGTFCPTVFPKMESSKPLTMIDKTVTLQYDSDTRKYILFV |
| | | PRVTPKYSVNKEKNSCGIDPGLRDFLTVYSENETQSICPIEIVVNTTKNEY |
| | | KKIDKINEIIKTKPNLNSKRKKKLNRGLRKYHRRVTNKMKDMHYKVSHE |
| | | LVNTFDKICIGKLNVKSILSKANTVLKSALKRKLATLSFYRFTQRLTHMG |
| | | YKYGTEVVNVNEYLTTKTCSNCGKIKDLGASKIYECESCGMYADRDEN |
| | | AAKNILKVGLKPWYKQK |
| 123 | Methanocaldococcus | MLFKFEGDKIKIITAPRKFITINLVVSDYQKKFIEEWKNGNFKIGEVIIKKD |
| | jannaschii | SIIIPFKKVVNPKNFEHIMTIDINEKNITYSIFDKDGNVIKTTRLDVYKLKRI |
| | MJ0751 | HENFSKKREKIQKKLSNKPMKLKTLMEKYSGREKRKVEDYLHKISKFLIS |
| | (Q58161.1) | EALKYNVKILMEDLTNIREAVNKKSKNFRRRLNRWNFSKLQFFIEYKAK |
| | | WDGLDVEYVNPSRTSKLCPICGCKLDPNGQRLLKCNNCNLVFDRDVVA |
| | | TFNLFKKSQDVGSFRSPERSLMKSSY |
| 124 | Aquifex aeolicus | MKVYKRLYELLGRGYKPLKSARLIKRGKDYYIGITLQKAVKEKKIKKPR |
| | aq_aa05 | YVINVDLNVQRNLACIGIFEVDWEKRESKLYGIKFVNGKLLRLVYKRDY |
| | | LFEEIRKKQRQTGRSPQVGDNSRLWKKVNNLNRDIALKVAKEISDIAREF |
| | | SEKGEVIVVFEKLKGLRGRKGRSKKLNRKINFWMRRKIQERVKELGLEE |
| | | GFGLDFVYPHYTSKKCSKCGYEGERFSPSGSKALFLCKKCGYVVNADVN |
| | | AVFNQHFLYLSHLLNGGGKARPVVRVGTSLKSPSREGHNLSGVPKATTT |
| | | FFYISSLLSTF |
| 125 | Acanthamoeba | MKEAVKTVKPKVPAKKRIVTGSKTKKKVFVKKKPPAKKPPDKKPLKKT |
| | polyphaga | TKKTVKKVINRPKSIHIPNKDLKIFKWIPTPKKEFTEIETNSWYEHRKFENP |
| | mimivirus | NKSPIQTYNKIVPVVPPESIKQQNLANKRKKTNRPIVFISSEKIRIYPTKEQ |
| | MIMI R854 | QKILQTWFRLFACMYNSSIDYINSKKVVLESGRINVAATRKVCNKISVRK |
| | | ALKTIRDNLIKSTNPSIMTHIMDEAIGLACSNYKTCLTNYIEGQIKKFDIKP |
| | | WSISKRRKIIVIEPGYFKGNSFCPTVFPKMKSSKPLIMIDKTVTLQYDSDTR |
| | | KYILFVPRVTPKYSVNKEKNSCGIDPGLRDFLTVYSENETQSICPIEIVVNT |
| | | TKNEYKKIDKINEIIKTKPNLNSKRKKKLNRGLRKYHRRVTNKMKDMHY |
| | | KVSHELVNTFDKICIGKLNVKSILSKANTVLKSALKRKLATLSFYRFTQRL |
| | | THMGYKYGTEVVNVNEYLTTKTCSNCGKIKDLGASKIYECESCGMYAD |
| | | RDENAAKNILKVGLKPWYKQK |
| 126 | Lactobacillus | MKRMSSLAYHFGLKVRFYPSSKQKKIIKLNYDAQRFVYNSYVGRNRNN |
| | crispatus tnpB | YRARGYLAVRQYQVMPFAFSVLNNYETKLAEEVVANNELLAKPKNIRD |
| | | AYSFLRVKEIDSLAIANAIQNYQKAWNNYRKLGHGIPTFHKKRSDWSYQ |
| | | TNCQYPKQTEAFLDNGTARFIDAKHVKLPKLGVVRIASFRKLIKERFLNHI |
| | | PTRIGTVTIKKTADDQFYLSMQLGSDIAFVKELPKTQSQIGIDLNLDNFLT |
| | | ASNGAMVANPRFYRKTKKKLAHAQRVLSRRQRRAKKEGRNLRLAKNY |
| | | QKQRLAVAKLHDKIRRQRNDFLQVLSTALIKNHDLVVAEELRSKNLLKN |
| | | HALSQAISDVGWRSFLNMLAYKADLYGKEFKTIDPKYTTQRCHQCGSIM |
| | | GQNGYKKLTLKDREWTCPICQTHHIRDWNAAVNILEKGLGNWQNSKIK |
| | | KAA |
| 127 | Yersinia ruckeri | MSTFYAVRRLCLVLGLHYSGYYQWLKRPQSLRARQDERQTGLIKQLWL |
| | tnpB_2 | ESGTVYGYRKIWLDMKDLGERAGRNRIARLMRLAELASQTGYRKRRYY |
| | | GGGKTSVANPNYLERQFIVPTPNTHWVSDMTYIRTHEGWLYLAVVLDLF |
| | | SRRVIGWSMGGRMTAELVMDALLMAVWRRKPTAKVLIHSDQGSQYTSE |
| | | RCQHFMTAHNLKSSMSRRGNCHDNAVAESFFQLLKRERIKKRIYKNREE |
| | | AKADIFDYVEMFYNAKRRHSACEDQPPAVYESAWFMRHATV |
| 128 | Bacteroides | MKSSMAMIKHKKELVEYARHLKLPNLAEHVGVILHEAQEKQLTYSEFLA |
| | fragilis tnpB | NCLAREIQGRERKSYLTRLKLSGLPAKYDLDLYDYDRAEGMDNRRLREL |
| | | RELVWVNQAYNLLLVGPSGTGKTFIAAGLVYEAVKAGYEAYLMTLEEL |
| | | LTCLKTKEVSAHAMKTYKRIMKARLLAIDDATLFPLKREEAVLLFKLVN |
| | | DFQERTSLIITANKALTRWLETLEDEAVTAALLDRLLYCCEIIRLGGTSYR |
| | | MQNRKTIFSNQNTDIGT |
| 129 | Cohnella zeiphila | MQITFQFRLYPTPEQEQQMNRTLDLCRKLYNRAKEQRDTANNQEGKTV |
| | tnpB | TNTMQQNELPAFKKEFPAYKEVHSQVLQDCLRRLDHAYQRFFRGEAGFP |
| | (MBB6733828.1) | HYKSADRYLSFTYPQPGAVTKTFAKGGYVYLPKIGLVRMRAHRPFDRAN |
| | | VTQINVKRYADGWMAGIGVKVETAEASQTMETAVGIDVGLRTFPALSD |
| | | ETKVNSPRHLRKAEKRLRRAQRRLSRKQRGSRNRQKAKRKVARIHQKV |
| | | ARQRKDFLHKQSHRIVRSHDLIAVEQLKVKNMIRNRRLSKSISDAGWRK |
| | | FITYLSYKAERQGKRFVQVPAHGTSQTCVCGANVPKTLATRLHECKRCG |
| | | LVQDRDIVSAKVILQRALEIAA |
| 130 | Moorena sp. | MKARYKYRFYPTDQQKQDLARLFGCVRVVWNDALAICKKYKKVLKSG |
| | SIO4A3 tnpB | DLQKLVITQAKKTEERKWLSEVSVVPLQQSVADLGVAFKNFFESHNGKR |
| | (NEO44226.1) | KGRKVRSPRFKKRSNNQSARLTRRGFSIQGDGVYLAKIGVVKPIWSRELP |
| | | SDPSSVTIILDCANNYFLSFVVDIKPVQVDAKNQSIGIDLGIKTFAVMSDG |
| | | GHVKSPDYSKLDRKLRKQTRKFARQAKGSNRREQTRIKIARLNKKIANK |
| | | RKDFLHKFSTRIVSENQVIVLEDLNVSGMVKNRKLARAISQQGWSEFRSL |
| | | AQAKSDKYGRDFRVISRWEPTSQICSDCGFKWGKLDLSVRSILCVNCGTE |
| | | QDRDINAARNIEKVGMGHRHDSKWARRGDKTVETVGHATRTAAHPDD |
| | | LSRIAVLLGR |
| 131 | Streptomyces | MQLRYNYRAYPVASQRRALARAFGCARVVWNDCLRDRKEAHAAGLPY |
| | boncukensis tnpB | MKSAELSRLRITQAKHTEERAWLADVSAVVLQQSLRDLDTAYKNFFDSV |
| | (NGO73097.1) | KGKRQGRKTGPPRYKSKKDTRQSIRLNTNAFSLQGDGTVYVAKVGNLN |
| | | VTWSRRLPAAPTSLTVTKDSSGRYVLSFVVDTEPDILPEVEAESGIDLGLS |
| | | AFAVLSDGRKIDSPRFLRRAEKKLERLQREHSRKAKGSKNRAKARIKVA |
| | | RQHAKVADRRRDWHHKASTQIIRDNQAVYVEDLAVSGLGRTRLAKSVH |
| | | DAGWSAFTAMLEYKAVKHGRTFAKVDRTFPSSQVCSVCGFRDGPKPLH |
| | | VRAWTCGECATVHDRDHNAARNVLFEGRRIVATGRVETLNACGVPVRR |
| | | AHVPAQRGETGSPRKGQTTQAGITGLQAREHVKVAV |
| 132 | Symploca sp. | MKARYRYRIYPTVGQQMALARLFGCVRVVWNDALAFCIAQYQGGKKK |
| | SIO2C1 tnpB | PKNAELQKQFITQAKRIIERTFLSQVSCVPLQQSLNDLEQAYQNFFKSCKG |
| | (NEP14615.1) | QRKGKPVKPPRFKKRHAKQSARFTQRGFSLKGDCLKLAKIGKLKIAWSR |
| | | PLPAEPTSVTIIKDAAGRYFVSFVVEIVSEVLPNTEKSVGIDLGISTFATLD |
| | | NGQKIDAPKPLKKRINRLRRLSKNLSRKTRGSKRYERARKRKAKLQAKL |
| | | KDTRTDFLHKLSTQIIRENQTIVLEDLNVAGMVKNRKLSKAISDLGWRQF |
| | | RTYLEGKVQKYGREFRVISRWEPTSQTCSECGFRGGKLDLSIREWTCLNC |
| | | GITHDRDVNAAKNIKLAGGHSESLNGRGGRRKTSAKGAAAREASTHREY |
| | | VQLSLFG |
| 133 | Staphylococcus | MNASSKRKIISQSEISKKIAVMNEEMQGFWANNSWDIRKCPHPSAIELSK |
| | aureus (strain | NPALRNRWVRFERVKNLWLRTELKYFYFYHLNNGIWNAKTVWIRKGTV |
| | Mu50/ATCC | INKMLDFLDLKYPSITSITEVPIEKAMTEYRTYLTKRGVRITTTNYKITAN |
| | 700699) tnpB1 | QEKTPVKANSYYVTNLKQFMEFYENFYFDGEEWDKDVWDRRNLPLPDD |
| | | KVNPTQYEYTINFKGFRNTYFKQLVKRYCKLRLNVDSFSYVSDIAQRLKE |
| | | FFNFLDMKFKQVQRVHQLTRVEIEAYLSELNMMGIKPSTITGRISILEGLF |
| | | STLLRLEWDDVPSKILIYSEDYPKIPRAKPRFIDEFVLEQLNSHLDKLPEYI |
| | | ATMTMIVQECGMRISELCTLKKGCLLEDKDGDFFLKYYQWKMKKEHIV |
| | | PISKEVALLIKVREDKVSEEFPDSEYLFPRKDGSPLKQETFRGELNKLAYE |
| | | QNIVDKSGEIYRFHAHAFRHTVGTRMINNGMPQHIVQKFLGHESPEMTSR |
| | | YAHIFDETLKNEFTKFQEKLVTNNGDVLDLDEDNEVDDVELQWFKKNIN |
| | | AQVLPNGYCRLPVVAGGCPHANACLDCTHFCTSKQFLPQHEEQLERTEE |
| | | LLAIAKDKQWQRQVETNSRVKERLEQIIGSLTG |
| 134 | Lactobacillus | MKRMSSLAYHFGIKLRFYPSSKQKKLIKLNYDAQRFVYNSYVGRNRSSY |
| | kefiranofaciens | HAKRYFLVKQSKTMPFAFSILNRYETKLAETVVANNELLAKPKNIRDAY |
| | tnpB | SFLRIKEIDSLALANAIQNYHKAWNNYRKIGHGIPTFHKKRSDWTYQTNC |
| | | QYIKQEEAYLDNGTAKFIDAKHVKLPKLGVVRIAGLRKLIKERLFKHIPT |
| | | RIGTVTIKKTAEDQFYLSMQSGSDLAFVKELPKTQNQIGIDLNLDNFLTNS |
| | | NGAMVANPHFYRQAEKKLAHAQRVLSRRQRRAKKARRNLRTAKNYQK |
| | | QRLIVAKLHDRIRRQREDFLQVLSTALIKNHDLVVAEELRSKNLLKNHAL |
| | | SQAISDVGWRKFLNMLAYKGRLYGKEFITIDPKYTTQRCHACGTIMGHN |
| | | GYKKLKLKDREWTCPICHTYHIRDWNAAVNILQKGLGSWQNPKKQAT |
| 135 | Helicobacter suis | MLKAIKFRIYPTIEQKTLIHKHFGCARVVYNYFLAYRQKQYAQGIRENYF |
| | tnpB | SMQKALTTLKKQEAYAYLSECNSQSLQMALRQLTTAFDRFFSKLADYPR |
| | | FKSKKHSKQSFCVPQHLEMDLGNNQVKLPKFKEAIKAKFHRHLPTNSIV |
| | | KQGFISCVADKYYLSISYEDNEPEPKPTTIRKAVGLDMGLESLVIASNGVF |
| | | YPYKKFFQNLQTKLTKAQRRLSKKLKGSNNRKKQAKKVAQIHASIRNSR |
| | | EDYLHKISNEITNQYDLIAVETLKVRNLVKNHKLAKSIANASWSRLISLLE |
| | | YKAGWKGKTLIKIDQYFPSSQICSTCGSNTGKKPLPIRNFIYPCCQTYHHR |
| | | DLNASINIRNYALGMLDERHAIKVDKTRVGITRSYACGDSANGAVTKYG |
| | | YILDTASYGSLKQEAHPSLAGG |

TABLE B
TnpB ncRNA sequences

| TnpB | Accession | | | | | | |
|---|---|---|---|---|---|---|---|
| SEQ ID | No. | Protein coding + reRNA | SEQ ID | Protein coding | SEQ ID | reRNA containing region | SEQ ID |
| 1 | WP_ | ATGATAAGGAATAAGGCTTTCGTGGT | 209 | ATGATAAGGAATAAGGCTTTC | 240 | ATCTTGCCGTCCGTACATGGA | 271 |
| | 010887311.1 | CAGGCTGTACCCAAATGCGGCTCAG | | GTGGTCAGGCTGTACCCAAAT | | CTTGCCCGAACTGTGGGGAAA | |
| | | ACTGAACTGATTAACCGCACGCTGG | | GCGGCTCAGACTGAACTGATT | | CCCATGACCGAGACGAGAAC | |
| | | GTAGCGCAAGGTTCGTCTACAACCAC | | AACCGCACGCTGGGTAGCGCA | | GCTGCGCTGAACATTCGGCGT | |
| | | TTCCTTGCCCGTCGCATTGCGGCCTA | | AGGTTCGTCTACAACCACTTCC | | GAAGCGTTGGTGGCTGCGGG | |
| | | CAAGGAAAGCGGGAAGGGACTGACC | | TTGCCCGTCGCATTGCGGCCTA | | AATCTCAGACACCTTAAACGC | |
| | | TACGGGCAAACGAGTAGCGAACTGA | | CAAGGAAAGCGGGAAGGGAC | | TCATGGAGGCTATGTCAGACC | |
| | | CCCTTCTGAAGCAGGCTGAAGAAAC | | TGACCTACGGGCAAACGAGTA | | TGCTTCGGCGGGCAATGGTCT | |
| | | CTCCTGGCTCTCGGAAGTAGATAAGT | | GCGAACTGACCCTTCTGAAGC | | GCGAAGTGAGAATCACGCGA | |
| | | TTGCTTTGCAGAACTCGCTGAAAAAC | | AGGCTGAAGAAACCTCCTGGC | | CTTTAGTCGTGTGAGGTTCAA | |
| | | CTTGAGACCGCGTACAAGAACTTCTT | | TCTCGGAAGTAGATAAGTTTG | | GCCGGCAATCACGCCCGAGTT | |
| | | TCGGACTGTGAAGCAGTCCGGTAAA | | CTTTGCAGAACTCGCTGAAAA | | GCTGCCCGAGCCGTCGCACGC | |
| | | AAGGTAGGATTCCCACGTTTCAGAA | | ACCTTGAGACCGCGTACAAGA | | CAGCACCTTGACGGCGATCAG | |
| | | AGAAGCGCACGGGAGAGTCCTACCG | | ACTTCTTTCGGACTGTGAAGCA | | CGACACGCCCTTGGCGACGCC | |
| | | GACTCAATTCACCAACAACAACATCC | | GTCCGGTAAAAAGGTAGGATT | | GTAGGTGCTGCTGCCCACCGT | |
| | | AAATTGGGGAAGGTAGGCTCAAACT | | CCCACGTTTCAGAAAGAAGCG | | GCCGGCGACGTGGGTGCCGT | |
| | | TCCTAAGCTGGGATGGGTGAAAACC | | CACGGGAGAGTCCTACCGGAC | | GGCCGTTGCAGTCGCTATCGT | |
| | | AAGGGCCAGCAGGATATTCAAGGGA | | TCAATTCACCAACAACAACAT | | TGCCGTCGCCCGACTGGTTGG | |
| | | AGATTCTGAATGTCACTGTGCGCCGT | | CCAAATTGGGGAAGGTAGGCT | | TGCCCCACACGGCGCGGCCAC | |
| | | ATTCACGAAGGCCATTACGAAGCGT | | CAAACTTCCTAAGCTGGGATG | | CGAACGCCGTATGACTAGTGT | |
| | | CCGTTCTCTGTGAAGTCGAGATTCCC | | GGTGAAAACCAAGGGCCAGCA | | TGATGCCGGTGTCGATGATGT | |
| | | TACCTGCCTGCGGCTCCCAAGTTTGC | | GGATATTCAAGGGAAGATTCT | | ACGCCTTCACGCCGCTGCCGG | |
| | | AGCGGGTGTGGATGTCGGCATCAAG | | GAATGTCACTGTGCGCCGTATT | | TGGTGCCATAGGTGTAGCTGC | |
| | | GATTTTGCCATCGTGACCGATGGCGT | | CACGAAGGCCATTACGAAGCG | | TGTCGAGCGGCAGGCTGCGCT | |
| | | GAGGTTTAAGCATGAACAGAATCCG | | TCCGTTCTCTGTGAAGTCGAGA | | | |
| | | AAATATTACCGCTCCACCCTGAAAAG | | TTCCCTACCTGCCTGCGGCTCC | | | |
| | | ACTTCGTAAAGCTCAGCAAACCCTGT | | CAAGTTTGCAGCGGGTGTGGA | | | |
| | | CCAGACGGAAGAAGGGCAGCGCACG | | TGTCGGCATCAAGGATTTTGCC | | | |
| | | TTACGGGAAAGCGAAAACCAAGCTG | | ATCGTGACCGATGGCGTGAGG | | | |
| | | GCTCGGATTCACAAGCGCATTGTCAA | | TTTAAGCATGAACAGAATCCG | | | |
| | | TAAGCGTCAGGATTTCCTTCACAAGC | | AAATATTACCGCTCCACCCTG | | | |
| | | TCACCACCTCCCTGGTGCGTGAGTAC | | AAAAGACTTCGTAAAGCTCAG | | | |
| | | GAAATCATCGGAACCGAACACCTTA | | CAAACCCTGTCCAGACGGAAG | | | |
| | | AACCCGACAACATGCGGAAAAATCG | | AAGGGCAGCGCACGTTACGGG | | | |
| | | CCGCCTTGCACTGAGCATCAGTGATG | | AAAGCGAAAACCAAGCTGGCT | | | |
| | | CGGGCTGGGGTGAGTTCATCCGGCA | | CGGATTCACAAGCGCATTGTC | | | |
| | | GTTGGAATACAAGGCAGCGTGGTAC | | AATAAGCGTCAGGATTTCCTTC | | | |
| | | GGGCGACTGGTATCTAAAGTCAGCC | | ACAAGCTCACCACCTCCCTGG | | | |
| | | CCTACTTTCCATCTAGCCAGTTGTGT | | TGCGTGAGTACGAAATCATCG | | | |
| | | CATGACTGCGGATTCAAGAATCCCG | | GAACCGAACACCTTAAACCCG | | | |
| | | AAGTGAAGAATCTTGCCGTCCGTACA | | ACAACATGCGGAAAAATCGCC | | | |
| | | TGGACTTGCCCGAACTGTGGGGAAA | | GCCTTGCACTGAGCATCAGTG | | | |
| | | CCCATGACCGAGACGAGAACGCTGC | | ATGCGGGCTGGGGTGAGTTCA | | | |
| | | GCTGAACATTCGGCGTGAAGCGTTG | | TCCGGCAGTTGGAATACAAGG | | | |
| | | GTGGCTGCGGGAATCTCAGACACCTT | | CAGCGTGGTACGGGCGACTGG | | | |
| | | AAACGCTCATGGAGGCTATGTCAGA | | TATCTAAAGTCAGCCCCTACTT | | | |
| | | CCTGCTTCGGCGGGCAATGGTCTGCG | | TCCATCTAGCCAGTTGTGTCAT | | | |
| | | AAGTGAGAATCACGCGACTTTAGTC | | GACTGCGGATTCAAGAATCCC | | | |
| | | GTGTGAGGTTCAAGCCGGCAATCAC | | GAAGTGAAGAATCTTGCCGTC | | | |
| | | GCCCGAGTTGCTGCCCGAGCCGTCGC | | CGTACATGGACTTGCCCGAAC | | | |
| | | ACGCCAGCACCTTGACGGCGATCAG | | TGTGGGGAAACCCATGACCGA | | | |
| | | CGACACGCCCTTGGCGACGCCGTAG | | GACGAGAACGCTGCGCTGAAC | | | |
| | | GTGCTGCTGCCCACCGTGCCGGCGAC | | ATTCGGCGTGAAGCGTTGGTG | | | |
| | | GTGGGTGCCGTGGCCGTTGCAGTCGC | | GCTGCGGGAATCTCAGACACC | | | |
| | | TATCGTTGCCGTCGCCCGACTGGTTG | | TTAAACGCTCATGGAGGCTAT | | | |
| | | GTGCCCCACACGGCGCGGCCACCGA | | GTCAGACCTGCTTCGGCGGGC | | | |
| | | ACGCCGTATGACTAGTGTTGATGCCG | | AATGGTCTGCGAAGTGAGAAT | | | |
| | | GTGTCGATGATGTACGCCTTCACGCC | | CACGCGACTTTAGTCGTGTGA | | | |
| | | GCTGCCGGTGGTGCCATAGGTGTAGC | | | | | |
| | | TGCTGTCGAGCGGCAGGCTGCGCT | | | | | |
| 44 | NP_ | ATGCCAATTTTTGCCGCCAAGAAAAG | 210 | ATGCCAATTTTTGCCGCCAAG | 241 | ACAAAGCTTCTCACTTTGAAA | 272 |
| | 986403.1 | ACAAGCTACACTGAAGAAGAAGCTT | | AAAAGACAAGCTACACTGAAG | | ATACCACAGTAATCGAAGTG | |
| | | GTTCTCCAGCAGGGTGATGAATTGAA | | AAGAAGCTTGTTCTCCAGCAG | | GATGAGGAATTTACGACAAA | |
| | | GAAGGAGAGTCTCAACATCCCGAGC | | GGTGATGAATTGAAGAAGGAG | | GACGTGCTCCTTGTGTGGTGT | |
| | | TGGGCGCCATTTAAGCTGATTGATCA | | AGTCTCAACATCCCGAGCTGG | | CCGCAGTAATATCGGTGCGTC | |
| | | CGGACAGGCCGCGAATGACCAGGTG | | GCGCCATTTAAGCTGATTGATC | | CAAAGTGTTTAAGTGCGATAA | |
| | | CTTATTCCTCAGAAGCCGGAGGTTCC | | ACGGACAGGCCGCGAATGACC | | TTGTGACGCGACTTTCGATAG | |
| | | GGGTGACGCGCTTAAGGATCATTGG | | AGGTGCTTATTCCTCAGAAGC | | AGACCACAATGCGGCCAAAA | |
| | | ACTCCAAAGACCGTAAGGATATATC | | CGGAGGTTCCGGGTGACGCGC | | ATATCCTTTTGAAGTACCTTG | |
| | | CCACTAGCAAACAGAAAGAGATACT | | TTAAGGATCATTGGACTCCAA | | GCGAGATAGATTAGTATGCTA | |
| | | ACACTTATGGCTTACGGCTACAAGAC | | AGACCGTAAGGATATATCCCA | | TATGTAGTGCTTCCTGTGTAT | |
| | | ATATGTACAACCTTGCTGTGGCCGAG | | CTAGCAAACAGAAAGAGATAC | | GCTGCAATGAATCGATGTTTC | |
| | | AGAATTCGTTTGGGAAAGGAGGCCA | | TACACTTATGGCTTACGGCTAC | | ATGAGTTAATTTTGTTTTTACC | |
| | | AGGAAAACGGGACAAAAGTCAAGAT | | AAGACATATGTACAACCTTGC | | TTTTCGGGAAATGCTGGCAAT | |
| | | GAAGCTCAAGCACCTACGCGAAGTT | | TGTGGCCGAGAGAATTCGTTT | | CTAATTAATCACTGATCTGGC | |
| | | ATGCCCTTAAAGAAGAACTTTGATTA | | GGGAAAGGAGGCCAAGGAAA | | ATACTAAGTTATGCAGCCATC | |
| | | CGAGCGCGACCCGAAGCTCACCTATT | | ACGGGACAAAAGTCAAGATGA | | TCTTCTGAGAACGGTTCGTCC | |
| | | TGAAGAACGTACCGTTTCTAATCAAG | | AGCTCAAGCACCTACGCGAAG | | GTCAAATGTATAAATACAGAC | |
| | | GACAATGGCCTACGTGATTTCGACAA | | TTATGCCCTTAAAGAAGAACT | | GCCATCGCTCTGTTGTTTGCA | |
| | | GGACTATCGGACCAACCTGGCCAAA | | TTGATTACGAGCGCGACCCGA | | CTTTCATATCTTCCTTTAGACC | |
| | | TCTAAAAACACGCTGAGACCCTTCAA | | AGCTCACCTATTTGAAGAACG | | TCGAGCCTGGCTTCTTTTCGA | |
| | | GATCCACTTTAAGAAAATAAAGGAT | | TACCGTTTCTAATCAAGGACA | | AGTTGGTCCTCGGGTCCCCGC | |
| | | AAGCAGAGCATGACAGTGGAGAAAA | | ATGGCCTACGTGATTTCGACA | | CAGCTGCTTAGCAGAGGTCGT | |
| | | AAGTATGGGGCCTCAGCCGTGGCAT | | AGGACTATCGGACCAACCTGG | | GAACATCATGAGGCGATG | |
| | | ATACCACGACGTATTCCGGAAAGGT | | CCAAATCTAAAAACACGCTGA | | | |
| | | GCATTTAGGAGTACCGAGCCACTGCC | | GACCCTTCAAGATCCACTTTAA | | | |
| | | AGAACAACTCCCACACGATTCAAGA | | GAAAATAAAGGATAAGCAGA | | | |
| | | CTAATACGCGAGCATGGAAAATGGT | | GCATGACAGTGGAGAAAAAAG | | | |
| | | TTTTAGTCATCCCATATGAGACAAGC | | TATGGGGCCTCAGCCGTGGCA | | | |
| | | GGCCTCATGCCGAAGAAAGAGGCTA | | TATACCACGACGTATTCCGGA | | | |
| | | TCAATGCAGTTGCCGTGGATCCTGGC | | AAGGTGCATTTAGGAGTACCG | | | |
| | | GTCAGAACATTCCTAACTACCTACGA | | AGCCACTGCCAGAACAACTCC | | | |
| | | CAGCAGCCAAAGGGTGCTAGAGATC | | CACACGATTCAAGACTAATAC | | | |
| | | GGTGAGGATGGGTATCTGAAGCTTC | | GCGAGCATGGAAAATGGTTTT | | | |
| | | AGCACCTGGACCGCAGATTGCGATC | | TAGTCATCCCATATGAGACAA | | | |
| | | CCTTCAGTCAGAGATCCACAAGCTTC | | GCGGCCTCATGCCGAAGAAAG | | | |
| | | GTAGCAAGGGAATTGTGCTATCCAA | | AGGCTATCAATGCAGTTGCCG | | | |
| | | GGAAGACCGAAAAAGGAAAATACGT | | TGGATCCTGGCGTCAGAACAT | | | |
| | | AACCGCAGGACTGTAATCAGCAGGC | | TCCTAACTACCTACGACAGCA | | | |
| | | TTTATCAGCGGAAGAAAAACCTGGC | | GCCAAAGGGTGCTAGAGATCG | | | |
| | | TAACGATGTTCACAGAAAGGCATCC | | GTGAGGATGGGTATCTGAAGC | | | |
| | | CATTTATTAGCGAGCAAATACAATTA | | TTCAGCACCTGGACCGCAGAT | | | |
| | | CATTCTGATTCCGGTACTCAATTTCC | | TGCGATCCCTTCAGTCAGAGA | | | |
| | | ATAACTTCAAAAAAACAAACCGCAG | | TCCACAAGCTTCGTAGCAAGG | | | |
| | | AGTTAAGGCGAATCTCGCAAATATG | | GAATTGTGCTATCCAAGGAAG | | | |
| | | AAGCACTGTCTCTTCGTGCAGCGCCT | | ACCGAAAAAGGAAAATACGTA | | | |
| | | GAAGAACAAAGCTTCTCACTTTGAA | | ACCGCAGGACTGTAATCAGCA | | | |
| | | AATACCACAGTAATCGAAGTGGATG | | GGCTTTATCAGCGGAAGAAAA | | | |
| | | AGGAATTTACGACAAAGACGTGCTC | | ACCTGGCTAACGATGTTCACA | | | |
| | | CTTGTGTGGTGTCCGCAGTAATATCG | | GAAAGGCATCCCATTTATTAG | | | |
| | | GTGCGTCCAAAGTGTTTAAGTGCGAT | | CGAGCAAATACAATTACATTC | | | |
| | | AATTGTGACGCGACTTTCGATAGAGA | | TGATTCCGGTACTCAATTTCCA | | | |
| | | CCACAATGCGGCCAAAAATATCCTTT | | TAACTTCAAAAAAACAAACCG | | | |
| | | TGAAGTACCTTGGCGAGATAGATTA | | CAGAGTTAAGGCGAATCTCGC | | | |
| | | GTATGCTATATGTAGTGCTTCCTGTG | | AAATATGAAGCACTGTCTCTTC | | | |
| | | TATGCTGCAATGAATCGATGTTTCAT | | GTGCAGCGCCTGAAGAACAAA | | | |
| | | GAGTTAATTTTGTTTTTACCTTTTCGG | | GCTTCTCACTTTGAAAATACCA | | | |
| | | GAAATGCTGGCAATCTAATTAATCAC | | CAGTAATCGAAGTGGATGAGG | | | |
| | | TGATCTGGCATACTAAGTTATGCAGC | | AATTTACGACAAAGACGTGCT | | | |
| | | CATCTCTTCTGAGAACGGTTCGTCCG | | CCTTGTGTGGTGTCCGCAGTAA | | | |
| | | TCAAATGTATAAATACAGACGCCATC | | TATCGGTGCGTCCAAAGTGTTT | | | |
| | | GCTCTGTTGTTTGCACTTTCATATCTT | | AAGTGCGATAATTGTGACGCG | | | |
| | | CCTTTAGACCTCGAGCCTGGCTTCTT | | ACTTTCGATAGAGACCACAAT | | | |
| | | TTCGAAGTTGGTCCTCGGGTCCCCGC | | GCGGCCAAAAATATCCTTTTG | | | |
| | | CAGCTGCTTAGCAGAGGTCGTGAAC | | AAGTACCTTGGCGAGATAGAT | | | |
| | | ATCATGAGGCGATG | | TAG | | | |
| 47 | XP_ | ATGACCACCTGTACTCAATATCAGGC | 211 | ATGACCACCTGTACTCAATATC | 242 | ATGATCTAGGATCTAATAAAA | 273 |
| | 003682879.1 | TAAGAAGAGAAAAAGATCAAATAGT | | AGGCTAAGAAGAGAAAAAGA | | TCTACAGCTGTTCAAGTTGCT | |
| | | AAGACAACCAAACCATATTGGAATG | | TCAAATAGTAAGACAACCAAA | | CCTCGGTTTTCGATAGAGACT | |
| | | AGGATGCGGCTCATATTTCCCAGCAA | | CCATATTGGAATGAGGATGCG | | TCAACGCAGCAAAGAACATC | |
| | | TGGGGCTTGAATACGATTCTACCAAC | | GCTCATATTTCCCAGCAATGG | | ATGCTCAAGTATATCTGCGAG | |
| | | GGCGGCTAAAAATCTTAGTCAGAAA | | GGCTTGAATACGATTCTACCA | | CATTTGATGGCAAGTGGTGGA | |
| | | ATCACATCTGACTCGTGGTTTAGCGT | | ACGGCGGCTAAAAATCTTAGT | | TCTTCCATTACCTCTGCTTCAA | |
| | | GGTTAAACATCCACAGGTGCCGGTTA | | CAGAAAATCACATCTGACTCG | | GTGGAGGAAATGGTGGATTC | |
| | | TCAATGACTCATTGAACTTGCCGCTT | | TGGTTTAGCGTGGTTAAACATC | | GCCATGATGGGTCCTGGCCCC | |
| | | AGCAGTGGTGTCAAAGAAGAGAATA | | CACAGGTGCCGGTTATCAATG | | TTCGTACGTTAGTACGTTGTA | |
| | | CCAGGGCTCGCAAAATTAAGGTCTAT | | ACTCATTGAACTTGCCGCTTAG | | AGTCCCTTGTGGATTCTCCCA | |
| | | CCGACCAATTCCCAGAGAGAAACGC | | CAGTGGTGTCAAAGAAGAGAA | | GGCGCTACCCGCGGAACGTAT | |
| | | TCAAAAAGTGGTTTAGGTGCCAGCGT | | TACCAGGGCTCGCAAAATTAA | | TACTAAGGGTATATATATAAT | |
| | | TACATATACAATAGAGCGCTGAAGC | | GGTCTATCCGACCAATTCCCA | | AAGGATAATATATAAAGTGCT | |
| | | TGCACCGCGATGGCATGGCAATGAC | | GAGAGAAACGCTCAAAAAGTG | | GCAAAAGATGAGGCTCGAAT | |
| | | CATAAAAGGTCTACGCCAGACTTTGC | | GTTTAGGTGCCAGCGTTACAT | | TTTTCACAAGTTATCGGTCCC | |
| | | TTAACAGAGATACTATTATTCTAGAA | | ATACAATAGAGCGCTGAAGCT | | AACGACCTACCAGTTAAGAA | |
| | | CCTCAGGAGCAGTGGCTTAACGACT | | GCACCGCGATGGCATGGCAAT | | AGACCAATTAAGGAATATGG | |
| | | ACAGTTACGATTTGAAAGACGAAGC | | GACCATAAAAGGTCTACGCCA | | TTACTTCCAGAGCTGTCACTT | |
| | | CCTTTGTGATTTGGTCAAGAACTACA | | GACTTTGCTTAACAGAGATAC | | TTATCAATAATTCTACACCTG | |
| | | GTTATAATATGGCAAAGTATAAGCA | | TATTATTCTAGAACCTCAGGA | | CTACTTTGGTGAAAAAAGAG | |
| | | GACTGGGATGCCTTTGAAGTTGAGGT | | GCAGTGGCTTAACGACTACAG | | ATTGACCTTGATAAATGTCAT | |
| | | TCAAGTCAAAGAAGACACCGATTCA | | TTACGATTTGAAAGACGAAGC | | GATGAAAATCAAATTGTTATA | |
| | | AACTCTTTCAGTGTTGGAAAAGCACT | | CCTTTGTGATTTGGTCAAGAAC | | CAGGTTTATGCTGCCAGTTTG | |
| | | GGAACAAGAGAACGAAGTCGTTTTG | | TACAGTTATAATATGGCAAAG | | | |
| | | GAGCGAAGTATTCTCGCTCTCGAATT | | TATAAGCAGACTGGGATGCCT | | | |
| | | TTCGTGGTACAGAGCCGCTCCCAGAG | | TTGAAGTTGAGGTTCAAGTCA | | | |
| | | GAATTGCCAGGAGATTCGAGGGTTC | | AAGAAGACACCGATTCAAACT | | | |
| | | AAAGGACAAGGAACAACAAATTCTA | | CTTTCAGTGTTGGAAAAGCAC | | | |
| | | CTTGGTAGTTCCACTTGCTGGTGAGG | | TGGAACAAGAGAACGAAGTCG | | | |
| | | AAATCGCTAGAAAAAGTCCCGCAGA | | TTTTGGAGCGAAGTATTCTCGC | | | |
| | | AAAGTTTATATTTATTGATCCCAGGG | | TCTCGAATTTTCGTGGTACAGA | | | |
| | | TGAGAACTTTCCTAACAGGATATGAC | | GCCGCTCCCAGAGGAATTGCC | | | |
| | | TCTAAGCAGACTGTGGTCGAGATGG | | AGGAGATTCGAGGGTTCAAAG | | | |
| | | GGAAGGACGCGGTTGTACGCATCGA | | GACAAGGAACAACAAATTCTA | | | |
| | | GAAGCTTAAGCGCCGACGACGGCAA | | CTTGGTAGTTCCACTTGCTGGT | | | |
| | | TTCCAGTCAAAACTTGCCAAGCTCAG | | GAGGAAATCGCTAGAAAAAGT | | | |
| | | GAAGCACAAGAAGAGGCAGAACCAT | | CCCGCAGAAAAGTTTATATTT | | | |
| | | CGGAAAGCCTTACATAGGCTCGACG | | ATTGATCCCAGGGTGAGAACT | | | |
| | | AAAAGATTCAGCATATTGTTCGTGAT | | TTCCTAACAGGATATGACTCTA | | | |
| | | ATGCATAAGAAAACAGCCTCTTATTT | | AGCAGACTGTGGTCGAGATGG | | | |
| | | GTGTAAAAACTACGAAAACATCTTTC | | GGAAGGACGCGGTTGTACGCA | | | |
| | | TCCCCAAATTGAATTACCATCAATGC | | TCGAGAAGCTTAAGCGCCGAC | | | |
| | | ACTAGACTGACCCGAAAGACAAGGT | | GACGGCAATTCCAGTCAAAAC | | | |
| | | CCAGCATGGCAACCATTGCTCATTGT | | TTGCCAAGCTCAGGAAGCACA | | | |
| | | TGTTTTCACGATCGCTTGACGATGAA | | AGAAGAGGCAGAACCATCGGA | | | |
| | | GGCAGAGCAGTTCCATAGTACTAGT | | AAGCCTTACATAGGCTCGACG | | | |
| | | GTTCATGAAGTTGAGGAGGATTACA | | AAAAGATTCAGCATATTGTTC | | | |
| | | CGTCGAAGACTTGTTCTAGTTGTGGC | | GTGATATGCATAAGAAAACAG | | | |
| | | AATTTAAAAGATGATCTAGGATCTAA | | CCTCTTATTTGTGTAAAAACTA | | | |
| | | TAAAATCTACAGCTGTTCAAGTTGCT | | CGAAAACATCTTTCTCCCCAA | | | |
| | | CCTCGGTTTTCGATAGAGACTTCAAC | | ATTGAATTACCATCAATGCACT | | | |
| | | GCAGCAAAGAACATCATGCTCAAGT | | AGACTGACCCGAAAGACAAGG | | | |
| | | ATATCTGCGAGCATTTGATGGCAAGT | | TCCAGCATGGCAACCATTGCT | | | |
| | | GGTGGATCTTCCATTACCTCTGCTTC | | CATTGTTGTTTTCACGATCGCT | | | |
| | | AAGTGGAGGAAATGGTGGATTCGCC | | TGACGATGAAGGCAGAGCAGT | | | |
| | | ATGATGGGTCCTGGCCCCTTCGTACG | | TCCATAGTACTAGTGTTCATGA | | | |
| | | TTAGTACGTTGTAAGTCCCTTGTGGA | | AGTTGAGGAGGATTACACGTC | | | |
| | | TTCTCCCAGGCGCTACCCGCGGAACG | | GAAGACTTGTTCTAGTTGTGGC | | | |
| | | TATTACTAAGGGTATATATATAATAA | | AATTTAAAAGATGATCTAGGA | | | |
| | | GGATAATATATAAAGTGCTGCAAAA | | TCTAATAAAATCTACAGCTGTT | | | |
| | | GATGAGGCTCGAATTTTTCACAAGTT | | CAAGTTGCTCCTCGGTTTTCGA | | | |
| | | ATCGGTCCCAACGACCTACCAGTTAA | | TAGAGACTTCAACGCAGCAAA | | | |
| | | GAAAGACCAATTAAGGAATATGGTT | | GAACATCATGCTCAAGTATAT | | | |
| | | ACTTCCAGAGCTGTCACTTTTATCAA | | CTGCGAGCATTTGATGGCAAG | | | |
| | | TAATTCTACACCTGCTACTTTGGTGA | | TGGTGGATCTTCCATTACCTCT | | | |
| | | AAAAAGAGATTGACCTTGATAAATG | | GCTTCAAGTGGAGGAAATGGT | | | |
| | | TCATGATGAAAATCAAATTGTTATAC | | GGATTCGCCATGATGGGTCCT | | | |
| | | AGGTTTATGCTGCCAGTTTG | | GGCCCCTTCGTACGTTAG | | | |
| 79 | XP_ | ATGATCGTCCCGGCAACGGTGCTCGC | 212 | ATGATCGTCCCGGCAACGGTG | 243 | TGGCCCATGAAACCCAGAAC | 274 |
| | 009520458.1 | CGCCGCAGTTGGGAAGAGGAAACGA | | CTCGCCGCCGCAGTTGGGAAG | | CATCTCCAGCTCAACTTTTAC | |
| | | GGCACCGGGTCGTCTCGTAACCAGC | | AGGAAACGAGGCACCGGGTCG | | CGGCGCTTCAAACAATACATT | |
| | | AGCGGCAGGCGAAGCGTGTGGCCCG | | TCTCGTAACCAGCAGCGGCAG | | ACGCTCCGCTACGGGCTGGCT | |
| | | TCGAGAACGGAACCAGGCTGCCGCG | | GCGAAGCGTGTGGCCCGTCGA | | GGCCGCGATCGCTACCTTGTG | |
| | | AAGAAGCGCAAGACGTCGTCGGGGG | | GAACGGAACCAGGCTGCCGCG | | CTCAAGGGTATCCTCGACCCC | |
| | | CTGTCACCACGAAGCGCACGCCGAA | | AAGAAGCGCAAGACGTCGTCG | | GAGTACGACGGAGACAACGA | |
| | | GACAACACCGCGCAAGCGCAAGACC | | GGGGCTGTCACCACGAAGCGC | | GATCGTGCTGGAGTACAGAG | |
| | | CCGGTGAAGCCCAAGCCGACGTGGA | | ACGCCGAAGACAACACCGCGC | | AATTGATCCCCCGCAATGATG | |
| | | CGTTCACGACTGTCAAGATGAAGCTG | | AAGCGCAAGACCCCGGTGAAG | | AAGGAGCGAGCTAGCGAGCT | |
| | | CGGACGTTCTGCAAGGACGAGGGTG | | CCCAAGCCGACGTGGACGTTC | | ACTCCCGCTTGTGCACATGTT | |
| | | TAGCGCTCGCTTGGGACTCGGTGCTG | | ACGACTGTCAAGATGAAGCTG | | CCTCACTTTCGTGAAGGACCA | |
| | | AGGGACATGAACAAGATGGTGGCGG | | CGGACGTTCTGCAAGGACGAG | | CAACCGCGAGCACGAAGGAG | |
| | | AGGCTTACGTGCTGGCCGCCGTCCAC | | GGTGTAGCGCTCGCTTGGGAC | | AACCGGGCTTCGAGATGGCA | |
| | | GTGGTTCGCTGCTGTGAGCAAGGTTT | | TCGGTGCTGAGGGACATGAAC | | AGGACGTACTCGCTACTACCA | |
| | | CGACATCCCCATCTTGGCGCGTTCCT | | AAGATGGTGGCGGAGGCTTAC | | ACGAAACGGGGCTTCGTCTGC | |
| | | TCTACCAGCAGTGCTTGTCGGCCGTG | | GTGCTGGCCGCCGTCCACGTG | | TCTCACTTCACCATGTCCTCTC | |
| | | AGTGGGGGGAACCTGCACGATCGAC | | GTTCGCTGCTGTGAGCAAGGT | | GCGGCTTGCGGGCGATGCTGG | |
| | | GGACGACCATGACTGAAGAGTTCCG | | TTCGACATCCCCATCTTGGCGC | | TTCGAAGTGGATATGATGTTC | |
| | | CGACTCGGTGGCGATCTACAGCGCGT | | GTTCCTTCTACCAGCAGTGCTT | | CGGAAGAGGGTAGCGACTGG | |
| | | GGCAAGGCGAGAGCCTGATCCCCAA | | GTCGGCCGTGAGTGGGGGGAA | | AACGCTGTCGCGGACAGCAT | |
| | | CAGGGACGCCATCAATCGTGGTTGG | | CCTGCACGATCGACGGACGAC | | GTGGCGGCGGCTCTTCAACAC | |
| | | GCACAGGTGGCAGCCGAGCGCATGG | | CATGACTGAAGAGTTCCGCGA | | GAACCTTGTCGAGACGTCCAA | |
| | | CCCATGAAACCCAGAACCATCTCCA | | CTCGGTGGCGATCTACAGCGC | | GCCGGACGGGTCTCCAAGTCG | |
| | | GCTCAACTTTTACCGGCGCTTCAAAC | | GTGGCAAGGCGAGAGCCTGAT | | CTT | |
| | | AATACATTACGCTCCGCTACGGGCTG | | CCCCAACAGGGACGCCATCAA | | | |
| | | GCTGGCCGCGATCGCTACCTTGTGCT | | TCGTGGTTGGGCACAGGTGGC | | | |
| | | CAAGGGTATCCTCGACCCCGAGTAC | | AGCCGAGCGCATGGCCCATGA | | | |
| | | GACGGAGACAACGAGATCGTGCTGG | | AACCCAGAACCATCTCCAGCT | | | |
| | | AGTACAGAGAATTGATCCCCCGCAA | | CAACTTTTACCGGCGCTTCAAA | | | |
| | | TGATGAAGGAGCGAGCTAGCGAGCT | | CAATACATTACGCTCCGCTAC | | | |
| | | ACTCCCGCTTGTGCACATGTTCCTCA | | GGGCTGGCTGGCCGCGATCGC | | | |
| | | CTTTCGTGAAGGACCACAACCGCGA | | TACCTTGTGCTCAAGGGTATCC | | | |
| | | GCACGAAGGAGAACCGGGCTTCGAG | | TCGACCCCGAGTACGACGGAG | | | |
| | | ATGGCAAGGACGTACTCGCTACTACC | | ACAACGAGATCGTGCTGGAGT | | | |
| | | AACGAAACGGGGCTTCGTCTGCTCTC | | ACAGAGAATTGATCCCCCGCA | | | |
| | | ACTTCACCATGTCCTCTCGCGGCTTG | | ATGATGAAGGAGCGAGCTAG | | | |
| | | CGGGCGATGCTGGTTCGAAGTGGAT | | | | | |
| | | ATGATGTTCCGGAAGAGGGTAGCGA | | | | | |
| | | CTGGAACGCTGTCGCGGACAGCATG | | | | | |
| | | TGGCGGCGGCTCTTCAACACGAACCT | | | | | |
| | | TGTCGAGACGTCCAAGCCGGACGGG | | | | | |
| | | TCTCCAAGTCGCTT | | | | | |
| 92 | NP_ | ATGGAAGTCGACACCGAGAATCCAG | 213 | ATGGAAGTCGACACCGAGAAT | 244 | TGACTGATGTTCAGGGTGGTC | 275 |
| | 077707.1 | ACCCACGCGCGAAGAAAAGATGCAC | | CCAGACCCACGCGCGAAGAAA | | AGCGTGTGCGACAGTGTACA | |
| | | TCGGAAGAGCGGTGTGGAACAATCG | | AGATGCACTCGGAAGAGCGGT | | ACTGTTTCCGTGGGCGTTGAA | |
| | | CTACACGCAGAAGGCGATGAAGAGT | | GTGGAACAATCGCTACACGCA | | AATCCCTGTCCTCTTTTCGAA | |
| | | CAAGCTCAACAAGACAGTCGTGTAT | | GAAGGCGATGAAGAGTCAAGC | | AACGGGGTGGCATTTAGGTGT | |
| | | ATGTCTGGATCTCGAGAGGACTCTGG | | TCAACAAGACAGTCGTGTATA | | GATAGGGATGCGAGTGCCAC | |
| | | TGAAATCTGTGATGCCAAAGTACATT | | TGTCTGGATCTCGAGAGGACT | | CCTCAACTTTTGTCTCGCGGG | |
| | | CGAGACCGCCTCGAGAGAATGTCCA | | CTGGTGAAATCTGTGATGCCA | | ATACTGTGGGTTGGTTCGCAA | |
| | | GGTACACCGGGTCCTTCAGGTACATC | | AAGTACATTCGAGACCGCCTC | | GTCGTGGCCAACTCATCTCCT | |
| | | GTTGGTCTATTCTCAAACTACGTCGT | | GAGAGAATGTCCAGGTACACC | | GAGACGTCAGTAAGAGGACA | |
| | | CATCAGGAGACTCGAGCAGGGCCTG | | GGGTCCTTCAGGTACATCGTTG | | GAAAGTAGTCCCTCTGGGACA | |
| | | CAACCTCCTGAGGTCAAGAAAGTCTT | | GTCTATTCTCAAACTACGTCGT | | TGGCCGGTAGATTGACGCAG | |
| | | CTACGATAGGTGTTGGGCCGCCATCA | | CATCAGGAGACTCGAGCAGGG | | ATGTAAGGCCAGGCCTCTCAT | |
| | | ACACCCGCATATCCGGAAAGAACAC | | CCTGCAACCTCCTGAGGTCAA | | TCACACGAGAGGTCTATTGTT | |
| | | GAGAAACGACTTCTCCGACCTACTCG | | GAAAGTCTTCTACGATAGGTG | | GGACGAGTACGTTGGTTGGAT | |
| | | ACGAGTTCATCGACCAGTCCGGTTTC | | TTGGGCCGCCATCAACACCCG | | CATTTTGGTCTTGTTAACTGT | |
| | | GACACATCTCTCTTCCCTCCCAGGGT | | CATATCCGGAAAGAACACGAG | | ATTCCTCCGTAATAGGGCGGA | |
| | | CCCCTGTCGACTCCAAGAAACCGTCA | | AAACGACTTCTCCGACCTACTC | | TGTAGCCACCTGTACATATTT | |
| | | CGAGAGAAATGGAGGTATCTGCCAA | | GACGAGTTCATCGACCAGTCC | | TCTTGTGCGCTCCTCTACAGG | |
| | | GAACTCTATTGTTGTGCACCTGGAGG | | GGTTTCGACACATCTCTCTTCC | | AGACACGTGCTTGTCTTGATT | |
| | | CCAAGATCAAGGGGTTCCTGAGGTTT | | CTCCCAGGGTCCCCTGTCGACT | | GAAGTCTGTTACAAGGTATTC | |
| | | AGGCTCATGAACGATTCCCAGTTGGC | | CCAAGAAACCGTCACGAGAGA | | TGCGCCGAGGTAATAGGTTAT | |
| | | ATTCCAACACCTCCCCGCGAAGGATC | | AATGGAGGTATCTGCCAAGAA | | TAGACCAGAGATGACAAAGG | |
| | | GATCGAGAATTATCGTCTCTCTGTCA | | CTCTATTGTTGTGCACCTGGAG | | CGGTGGCGGCGTAAGCGTAG | |
| | | AACGACTGTCTGGAACGAGAGATGA | | GCCAAGATCAAGGGGTTCCTG | | C | |
| | | GAGGAGACCTCGATCCCGGGTACGT | | AGGTTTAGGCTCATGAACGAT | | | |
| | | ACCCCACGTGCTACAAGTGAGGGAC | | TCCCAGTTGGCATTCCAACACC | | | |
| | | CTTCTCGCACAAGTTTATTCGACCGA | | TCCCCGCGAAGGATCGATCGA | | | |
| | | ACCGGACGTATCAGTGCTCAAGGTCT | | GAATTATCGTCTCTCTGTCAAA | | | |
| | | TGAAGAAGAAGCCCCATCTGTTCCTC | | CGACTGTCTGGAACGAGAGAT | | | |
| | | GAACTGATCAGCATGATCTCACACGT | | GAGAGGAGACCTCGATCCCGG | | | |
| | | TGCCGAGGAGGCGTCCGACAACAAC | | GTACGTACCCCACGTGCTACA | | | |
| | | AGGGACCTCCGAGAGGAAACCAAGA | | AGTGAGGGACCTTCTCGCACA | | | |
| | | CGAGATCCGACGAGGAAAAAGTGAG | | AGTTTATTCGACCGAACCGGA | | | |
| | | GAAGGCCTTCTATATGTCAGAGAGG | | CGTATCAGTGCTCAAGGTCTTG | | | |
| | | ATCAAGCGAGGACTGGTGGATCGAC | | AAGAAGAAGCCCCATCTGTTC | | | |
| | | CCAAGATGTGCACGGTCCTCCCTGTC | | CTCGAACTGATCAGCATGATC | | | |
| | | TGGAAGCTAGCCCCGTGCTTTCCATA | | TCACACGTTGCCGAGGAGGCG | | | |
| | | CTACTCTTCTTCCGTGGTCGGGAGCA | | TCCGACAACAACAGGGACCTC | | | |
| | | TTTTTCACAAGGAGGGTCACGACTTC | | CGAGAGGAAACCAAGACGAG | | | |
| | | TCGTCCGTCTCAAGGTTTATTTCCGA | | ATCCGACGAGGAAAAAGTGAG | | | |
| | | GCACTTCGACCTTGGGAGGGTTAATA | | GAAGGCCTTCTATATGTCAGA | | | |
| | | GGAAAGGGTACAAGCCGTCGGGATT | | GAGGATCAAGCGAGGACTGGT | | | |
| | | TAGGTCAGACGGCTACCAGGTTCAG | | GGATCGACCCAAGATGTGCAC | | | |
| | | GTGACCTTTATGGCCCTGGTATCCAA | | GGTCCTCCCTGTCTGGAAGCTA | | | |
| | | AAAGCCCCACGTTCCTGGTACGAAA | | GCCCCGTGCTTTCCATACTACT | | | |
| | | GATCTGGCCAAGTCCGGGTATCAGAT | | CTTCTTCCGTGGTCGGGAGCAT | | | |
| | | TCCCAAGAAGGTGGTCTCCCTGGAA | | TTTTCACAAGGAGGGTCACGA | | | |
| | | ACGCAAGAAAGGGGCTTGTTCGTGC | | CTTCTCGTCCGTCTCAAGGTTT | | | |
| | | TCTCCCAGGCGAGAAAGGACAGTCG | | ATTTCCGAGCACTTCGACCTTG | | | |
| | | GAAGATTGTCGCTGACCGACTTCATG | | GGAGGGTTAATAGGAAAGGGT | | | |
| | | CATACAACCTGACCGTCGTCGATCCA | | ACAAGCCGTCGGGATTTAGGT | | | |
| | | GGGTGCGCTTCAGTGGTTTCTGTGAG | | CAGACGGCTACCAGGTTCAGG | | | |
| | | GTCGTGTCCCCTGGAATTCTGCAGGT | | TGACCTTTATGGCCCTGGTATC | | | |
| | | GTGTCGGGAGCGTTCGCGAGAGAAG | | CAAAAAGCCCCACGTTCCTGG | | | |
| | | CACGGCCTGGGAGATGAAGGGAACC | | TACGAAAGATCTGGCCAAGTC | | | |
| | | GAGTACTCCGCGAAGTGTGGGAGGA | | CGGGTATCAGATTCCCAAGAA | | | |
| | | CCATGTTGGAAGGAAGAGAGAAGAA | | GGTGGTCTCCCTGGAAACGCA | | | |
| | | GCGGAGATCGAACGACGAGTACGGG | | AGAAAGGGGCTTGTTCGTGCT | | | |
| | | CGGTGTTTCCCCAGATTTTCCGCGGT | | CTCCCAGGCGAGAAAGGACAG | | | |
| | | CAAGAAGAAGACAGCCAGAAAGTCG | | TCGGAAGATTGTCGCTGACCG | | | |
| | | TCTTTTCTCGCGTACTGTCGTGTGGC | | ACTTCATGCATACAACCTGAC | | | |
| | | GGCCGAAACGTTCAAGGTTATGTTCG | | CGTCGTCGATCCAGGGTGCGC | | | |
| | | CGGAAAAGATGAAGAGGGCGAGGA | | TTCAGTGGTTTCTGTGAGGTCG | | | |
| | | AGAGGTCGAGGTTCCACTCATCGAG | | TGTCCCCTGGAATTCTGCAGGT | | | |
| | | GCTTGTACAGAAGACTGTTGACAAG | | GTGTCGGGAGCGTTCGCGAGA | | | |
| | | CTGGCTTCGAACATAGCTGCGTGCCC | | GAAGCACGGCCTGGGAGATGA | | | |
| | | TTCCGACCGCAAGAGCATTGTTTTGT | | AGGGAACCGAGTACTCCGCGA | | | |
| | | TCGGGAACGGCAGCTTCAGGGCAAA | | AGTGTGGGAGGACCATGTTGG | | | |
| | | GAAAGGGCACGCCTCCGCGCCTCGA | | AAGGAAGAGAGAAGAAGCGG | | | |
| | | AAGAAGCTCGTGAGGGCTATCTGTTC | | AGATCGAACGACGAGTACGGG | | | |
| | | GAGGGTGAACGTGGGGATGTTGGAC | | CGGTGTTTCCCCAGATTTTCCG | | | |
| | | GAGTTCAGGACTTCCAAGATGTGCCC | | CGGTCAAGAAGAAGACAGCCA | | | |
| | | CGGGGGGTGTGGCGGGGAGATGACT | | GAAAGTCGTCTTTTCTCGCGTA | | | |
| | | GATGTTCAGGGTGGTCAGCGTGTGCG | | CTGTCGTGTGGCGGCCGAAAC | | | |
| | | ACAGTGTACAACTGTTTCCGTGGGCG | | GTTCAAGGTTATGTTCGCGGA | | | |
| | | TTGAAAATCCCTGTCCTCTTTTCGAA | | AAAGATGAAGAGGGCGAGGA | | | |
| | | AACGGGGTGGCATTTAGGTGTGATA | | AGAGGTCGAGGTTCCACTCAT | | | |
| | | GGGATGCGAGTGCCACCCTCAACTTT | | CGAGGCTTGTACAGAAGACTG | | | |
| | | TGTCTCGCGGGATACTGTGGGTTGGT | | TTGACAAGCTGGCTTCGAACA | | | |
| | | TCGCAAGTCGTGGCCAACTCATCTCC | | TAGCTGCGTGCCCTTCCGACCG | | | |
| | | TGAGACGTCAGTAAGAGGACAGAAA | | CAAGAGCATTGTTTTGTTCGGG | | | |
| | | GTAGTCCCTCTGGGACATGGCCGGTA | | AACGGCAGCTTCAGGGCAAAG | | | |
| | | GATTGACGCAGATGTAAGGCCAGGC | | AAAGGGCACGCCTCCGCGCCT | | | |
| | | CTCTCATTCACACGAGAGGTCTATTG | | CGAAAGAAGCTCGTGAGGGCT | | | |
| | | TTGGACGAGTACGTTGGTTGGATCAT | | ATCTGTTCGAGGGTGAACGTG | | | |
| | | TTTGGTCTTGTTAACTGTATTCCTCCG | | GGGATGTTGGACGAGTTCAGG | | | |
| | | TAATAGGGCGGATGTAGCCACCTGT | | ACTTCCAAGATGTGCCCCGGG | | | |
| | | ACATATTTTCTTGTGCGCTCCTCTAC | | GGGTGTGGCGGGGAGATGACT | | | |
| | | AGGAGACACGTGCTTGTCTTGATTGA | | GATGTTCAGGGTGGTCAGCGT | | | |
| | | AGTCTGTTACAAGGTATTCTGCGCCG | | GTGCGACAGTGTACAACTGTT | | | |
| | | AGGTAATAGGTTATTAGACCAGAGA | | TCCGTGGGCGTTGAAAATCCC | | | |
| | | TGACAAAGGCGGTGGCGGCGTAAGC | | TGTCCTCTTTTCGAAAACGGGG | | | |
| | | GTAGC | | TGGCATTTAGGTGTGATAGGG | | | |
| | | | | ATGCGAGTGCCACCCTCAACT | | | |
| | | | | TTTGTCTCGCGGGATACTGTGG | | | |
| | | | | GTTGGTTCGCAAGTCGTGGCC | | | |
| | | | | AACTCATCTCCTGAGACGTCA | | | |
| | | | | GTAA | | | |
| 93 | NP_ | ATGGTGAGACCATCTTCGGATGGAA | 214 | ATGGTGAGACCATCTTCGGAT | 245 | GCCCGGGGTGTGGAGAGGAC | 276 |
| | 597947.1 | ACTCGCACAAGAAGAAGAAACACAA | | GGAAACTCGCACAAGAAGAAG | | ATGGTCGAGGACAAAGAACG | |
| | | GAAGATACCTCCGAGGGCACCTCCA | | AAACACAAGAAGATACCTCCG | | GAGAATCCGGAGTTGCAAAA | |
| | | AAGAACATCCCGGGCTTTCTGCAGAC | | AGGGCACCTCCAAAGAACATC | | ACTTTAAGATCGGATCACCGG | |
| | | GGTGGCGAGCTTGTACCCGAATCAAT | | CCGGGCTTTCTGCAGACGGTG | | AAGAGTCCTGTAGACTTCACC | |
| | | ACCTCACGGCCTCAGGAAATCTACCC | | GCGAGCTTGTACCCGAATCAA | | GCCTGACACCAGAGTTTGAAA | |
| | | AAAAGTTGCAAGGGTACTTACTGTTG | | TACCTCACGGCCTCAGGAAAT | | TGGATCGCGACGACGTAGGG | |
| | | CCAGCTTTTCTCGAGAGTCAGGTTCG | | CTACCCAAAAGTTGCAAGGGT | | TCGACTAATATCGGTATGCGT | |
| | | GAAAGGAAGTGTCTCAGCAACTTCG | | ACTTACTGTTGCCAGCTTTTCT | | GGTTTCGGCAAATTGCTCGGT | |
| | | GACGAGGACCGTGAGCGTCGGATTT | | CGAGAGTCAGGTTCGGAAAGG | | CAGGTGTGGTTTTAGGGATTT | |
| | | CGGGCGGCAACGACCGATGAGAAAC | | AAGTGTCTCAGCAACTTCGGA | | TTTCCGGATTTTCACTTGCGG | |
| | | GCGACACATTTCAGTCCAAGGTCAAT | | CGAGGACCGTGAGCGTCGGAT | | ATGATACTTTATAGTTGGTTT | |
| | | GATGCGGTCGATCATCTCTCCCGTGA | | TTCGGGCGGCAACGACCGATG | | TCCTAAAGTTGACAATGGTGA | |
| | | ACGAGTTCTGGCGTCTCTCTTCGCAA | | AGAAACGCGACACATTTCAGT | | GCGCCTCCACGACATCCTTTT | |
| | | ACTTTGTGTTCATGGAACGTCTTCAA | | CCAAGGTCAATGATGCGGTCG | | GCTATGCGCCTTCCGTAACGC | |
| | | AACGGGTCAGCCCTTCCGGAGCCCG | | ATCATCTCTCCCGTGAACGAGT | | GCTGACCTCCGTGGCCAAGAT | |
| | | ACAGGGCTTTTTTCAAGTCGTGCTTG | | TCTGGCGTCTCTCTTCGCAAAC | | GCTTGCTGTGGAAGCATCTTC | |
| | | TCATCATGCATCTTAAGCAGAGGAG | | TTTGTGTTCATGGAACGTCTTC | | TGTGGCAGGACCGTTGTACGT | |
| | | GGGGAAGTCTCAACGCCGACTTCGA | | AAAACGGGTCAGCCCTTCCGG | | GACTCGCTCTTTGGACTTGAC | |
| | | CCGTTTTTCCGAGATAACAGGTTTGA | | AGCCCGACAGGGCTTTTTTCA | | CTCGAGAACTGAAAGCAGTG | |
| | | CAAGACTCGTGCCTCCCAAAAGCAT | | AGTCGTGCTTGTCATCATGCAT | | CCTGCCAGAAGCAAGGGTCA | |
| | | GAACCTTGACCAACAACGAGAGCAC | | CTTAAGCAGAGGAGGGGGAAG | | AACTGCTCGATTGCTGGGAGA | |
| | | GAGGCACAGCGTATGGCGACGGCAA | | TCTCAACGCCGACTTCGACCGT | | CCTCCCACATCTCCGTCCACA | |
| | | CGTCGACGCGTGCAACACACCACGC | | TTTTCCGAGATAACAGGTTTGA | | ACAACTCGGTCGAACAGCTTT | |
| | | GGAAAAGAGGAGGATTTCGATCACG | | CAAGACTCGTGCCTCCCAAAA | | T | |
| | | AGGTGGTTTCTGCGAGGGCGGATATC | | GCATGAACCTTGACCAACAAC | | | |
| | | TCGAGGCGACCTTGATTGCAAGAAG | | GAGAGCACGAGGCACAGCGTA | | | |
| | | TACGCGACGAAGCTCTACAAATTGG | | TGGCGACGGCAACGTCGACGC | | | |
| | | CGGAGAAGATCATCACAAACTTCGA | | GTGCAACACACCACGCGGAAA | | | |
| | | CGGCACACCCGAAGACACACAAAAG | | AGAGGAGGATTTCGATCACGA | | | |
| | | CTTCGAATAATGTCACGAGCGATAG | | GGTGGTTTCTGCGAGGGCGGA | | | |
| | | ACCCTGGTACAGACACCAACATCATC | | TATCTCGAGGCGACCTTGATTG | | | |
| | | ATCGACTTCGCATGGCAGGAGAAGG | | CAAGAAGTACGCGACGAAGCT | | | |
| | | AGTTCTCTGATCGAGACCGTGGAGAC | | CTACAAATTGGCGGAGAAGAT | | | |
| | | GGCAAGAGCGCAGAGACGATCAAAC | | CATCACAAACTTCGACGGCAC | | | |
| | | ACCTAAACTGGATGTTCAACACATAT | | ACCCGAAGACACACAAAAGCT | | | |
| | | GTCGCCGAGTCCAGAAGAATGTACG | | TCGAATAATGTCACGAGCGAT | | | |
| | | AGGAGGCCGTGTCCCGATCCCACTTG | | AGACCCTGGTACAGACACCAA | | | |
| | | TTCGCTCCCGGTAAAGACAAGGCGT | | CATCATCATCGACTTCGCATGG | | | |
| | | GCAGAAAGAAAAGGGACGAGTTCGT | | CAGGAGAAGGAGTTCTCTGAT | | | |
| | | GAAGCACGAGCTGGGCGAGGAGTTC | | CGAGACCGTGGAGACGGCAAG | | | |
| | | ACCTACGCTCCCCCCAAAGACACTGC | | AGCGCAGAGACGATCAAACAC | | | |
| | | GGTGCTACCCATTTGCTCAACGGGGG | | CTAAACTGGATGTTCAACACA | | | |
| | | CGGTGTTCATCTACATCGACATGAAG | | TATGTCGCCGAGTCCAGAAGA | | | |
| | | ACGCTCGGGTCATGGGGGCTCCTGCA | | ATGTACGAGGAGGCCGTGTCC | | | |
| | | CTCCAACGATGCTTGGTGGTACAAGG | | CGATCCCACTTGTTCGCTCCCG | | | |
| | | ACGTCCTCCGACCTTTTTCCAAAGAG | | GTAAAGACAAGGCGTGCAGAA | | | |
| | | GCAAATGTCAAGTGTCTCGTGAACA | | AGAAAAGGGACGAGTTCGTGA | | | |
| | | AGGAGAACGCCAGGTACGCGTCAAG | | AGCACGAGCTGGGCGAGGAGT | | | |
| | | CGAGCATGGCTACCTCTCGAGTCTGC | | TCACCTACGCTCCCCCCAAAG | | | |
| | | TAGAAAGTGGTCCGGACAAGTGCCC | | ACACTGCGGTGCTACCCATTTG | | | |
| | | CTGGATGATTGGCGAGAGTTTCTTGA | | CTCAACGGGGGCGGTGTTCAT | | | |
| | | CGGACGGTGTACAGATCAAGCTTTTG | | CTACATCGACATGAAGACGCT | | | |
| | | CTTAAGACACTAGAACACGGAAGGA | | CGGGTCATGGGGGCTCCTGCA | | | |
| | | AAGCTTTTTCCGGGTCCAGCGAGCTG | | CTCCAACGATGCTTGGTGGTA | | | |
| | | AACGAGGAGGGGTACAACAAGCTTC | | CAAGGACGTCCTCCGACCTTTT | | | |
| | | CCCGGGCTGACGTTCAGCTAGAAAG | | TCCAAAGAGGCAAATGTCAAG | | | |
| | | CCTCTTGGGAGAGGGAAGAGGGGTG | | TGTCTCGTGAACAAGGAGAAC | | | |
| | | TACAACATATCCAGCCTTTTGAAGTC | | GCCAGGTACGCGTCAAGCGAG | | | |
| | | GACAAGCATCAGGGAAGACGTCGTC | | CATGGCTACCTCTCGAGTCTGC | | | |
| | | GTGATGTCCGCTGACCCGGGCCAGG | | TAGAAAGTGGTCCGGACAAGT | | | |
| | | CCAAGGTTATCAACGTTTCTTCGGCT | | GCCCCTGGATGATTGGCGAGA | | | |
| | | ACGGCTAGAACTTGGGCGAAGCAAG | | GTTTCTTGACGGACGGTGTAC | | | |
| | | ACCCTGGCGTCATCCTCAAAACTTCG | | AGATCAAGCTTTTGCTTAAGA | | | |
| | | ACGTGTGTGTTCGGGGACGATTATCG | | CACTAGAACACGGAAGGAAAG | | | |
| | | TCGTGATACTCTTGCATCACGTTCAG | | CTTTTTCCGGGTCCAGCGAGCT | | | |
| | | ACGAGTACGAGACTTTCAGGCGCAA | | GAACGAGGAGGGGTACAACA | | | |
| | | GGGCCAGCCGTATGGAGTAGCCATC | | AGCTTCCCCGGGCTGACGTTC | | | |
| | | GATCGTCTTGGCAACGAGAAGAAAA | | AGCTAGAAAGCCTCTTGGGAG | | | |
| | | GAACTTCCTGTCTCGCAACGTTCATC | | AGGGAAGAGGGGTGTACAACA | | | |
| | | CAGTACTGCAGACGGTGGTGTGTAA | | TATCCAGCCTTTTGAAGTCGAC | | | |
| | | ATGGTCCGGTGTTATTCCGAGAGACT | | AAGCATCAGGGAAGACGTCGT | | | |
| | | TTGCAGACAGGAAGGAAGTTCATGA | | CGTGATGTCCGCTGACCCGGG | | | |
| | | GGTTTGACCGTTTCAGGGCTACGCAG | | CCAGGCCAAGGTTATCAACGT | | | |
| | | AAGACACTCGCCTTGATAGCAGACA | | TTCTTCGGCTACGGCTAGAACT | | | |
| | | AGTACATGGGACACGTAGAAGACAC | | TGGGCGAAGCAAGACCCTGGC | | | |
| | | TTGCAGGGTCATGTTGTTCGGCAAAG | | GTCATCCTCAAAACTTCGACGT | | | |
| | | CGTCTTTTCGTGCGCAGAAAGGGAG | | GTGTGTTCGGGGACGATTATC | | | |
| | | GGCATCTGCACCAAGGAAAGCTTTG | | GTCGTGATACTCTTGCATCACG | | | |
| | | ATCAGAGAGATGGCAAGTAGAGGCG | | TTCAGACGAGTACGAGACTTT | | | |
| | | TGGTGTTGATGGTGAACGAGTACAA | | CAGGCGCAAGGGCCAGCCGTA | | | |
| | | CACTTCCAAGATGTGCCCGGGGTGTG | | TGGAGTAGCCATCGATCGTCTT | | | |
| | | GAGAGGACATGGTCGAGGACAAAGA | | GGCAACGAGAAGAAAAGAAC | | | |
| | | ACGGAGAATCCGGAGTTGCAAAAAC | | TTCCTGTCTCGCAACGTTCATC | | | |
| | | TTTAAGATCGGATCACCGGAAGAGT | | CAGTACTGCAGACGGTGGTGT | | | |
| | | CCTGTAGACTTCACCGCCTGACACCA | | GTAAATGGTCCGGTGTTATTCC | | | |
| | | GAGTTTGAAATGGATCGCGACGACG | | GAGAGACTTTGCAGACAGGAA | | | |
| | | TAGGGTCGACTAATATCGGTATGCGT | | GGAAGTTCATGAGGTTTGACC | | | |
| | | GGTTTCGGCAAATTGCTCGGTCAGGT | | GTTTCAGGGCTACGCAGAAGA | | | |
| | | GTGGTTTTAGGGATTTTTTCCGGATT | | CACTCGCCTTGATAGCAGACA | | | |
| | | TTCACTTGCGGATGATACTTTATAGT | | AGTACATGGGACACGTAGAAG | | | |
| | | TGGTTTTCCTAAAGTTGACAATGGTG | | ACACTTGCAGGGTCATGTTGTT | | | |
| | | AGCGCCTCCACGACATCCTTTTGCTA | | CGGCAAAGCGTCTTTTCGTGC | | | |
| | | TGCGCCTTCCGTAACGCGCTGACCTC | | GCAGAAAGGGAGGGCATCTGC | | | |
| | | CGTGGCCAAGATGCTTGCTGTGGAA | | ACCAAGGAAAGCTTTGATCAG | | | |
| | | GCATCTTCTGTGGCAGGACCGTTGTA | | AGAGATGGCAAGTAGAGGCGT | | | |
| | | CGTGACTCGCTCTTTGGACTTGACCT | | GGTGTTGATGGTGAACGAGTA | | | |
| | | CGAGAACTGAAAGCAGTGCCTGCCA | | CAACACTTCCAAGATGTGCCC | | | |
| | | GAAGCAAGGGTCAAACTGCTCGATT | | GGGGTGTGGAGAGGACATGGT | | | |
| | | GCTGGGAGACCTCCCACATCTCCGTC | | CGAGGACAAAGAACGGAGAA | | | |
| | | CACAACAACTCGGTCGAACAGCTTTT | | TCCGGAGTTGCAAAAACTTTA | | | |
| | | | | AGATCGGATCACCGGAAGAGT | | | |
| | | | | CCTGTAGACTTCACCGCCTGAC | | | |
| | | | | ACCAGAGTTTGAAATGGATCG | | | |
| | | | | CGACGACGTAGGGTCGACTAA | | | |
| | | | | TATCGGTATGCGTGGTTTCGGC | | | |
| | | | | AAATTGCTCGGTCAGGTGTGG | | | |
| | | | | TTTTAG | | | |
| 94 | NP_ | ATGTCCGCATCACTTCTAACTGAGAC | 215 | ATGTCCGCATCACTTCTAACTG | 246 | AAACGCCAGTACCTTCTTCCA | 277 |
| | 478008.1 | ATCGTTGGAAGAGGAACAGAGAAGA | | AGACATCGTTGGAAGAGGAAC | | CTTCCTCTTCCTTTTGTACTCC | |
| | | GAGGTTAACAACAACACCAGTAAGG | | AGAGAAGAGAGGTTAACAACA | | TACTTCCATTACATGTGTCTT | |
| | | AAAATGATACAATGGAAAAAAAGAC | | ACACCAGTAAGGAAAATGATA | | GGGAGGAAAATTCGTCGACA | |
| | | TGAGACGGCTGCAACAACAGAAAAA | | CAATGGAAAAAAAGACTGAGA | | GGGACTTCAATGCAAGCACC | |
| | | GACCCAGAACCGTCTGTCAGTAAAA | | CGGCTGCAACAACAGAAAAAG | | AATATTGTTCATAAATTTCTA | |
| | | GGTCCAGAAATAAAGAACCCAAAAC | | ACCCAGAACCGTCTGTCAGTA | | GGGTTTTGGGATAAAAAGTTA | |
| | | AACTTCTACTGTTTACACTTCTGTAA | | AAAGGTCCAGAAATAAAGAAC | | ATGGAAAAGAAAGACAAGAT | |
| | | AGTGTTACCTTTCTTCCATAATCAAG | | CCAAAACAACTTCTACTGTTTA | | GCCGTTGAAGTATCACTTTAT | |
| | | AGTGAAAGTAGTAGAAGTAATGTCA | | CACTTCTGTAAAGTGTTACCTT | | TCGAGTTGCCTGAGAATAGGA | |
| | | CCTCAACCAAAGAAAGGTTTGAGGA | | TCTTCCATAATCAAGAGTGAA | | TGTAAAAAACTCGTGTCACTA | |
| | | GAGGTGTAAATCCGTAAGCAAGATG | | AGTAGTAGAAGTAATGTCACC | | GTGAAATAGTGGCGTAGCCCT | |
| | | ATGGTCAAAGGTTCACTGTTTTTGAG | | TCAACCAAAGAAAGGTTTGAG | | GAAGGATGAGAAGTATGCTA | |
| | | GTTAGTAGTGGACGAGTGTCTGAGA | | GAGAGGTGTAAATCCGTAAGC | | TTTTCGACCTTACGGTCATTG | |
| | | CGTTACAACCATCTAGAAGACGAAA | | AAGATGATGGTCAAAGGTTCA | | TGGTATATCAACTCTTGCCCG | |
| | | TCGATAAATGGCCAGATATGACGAA | | CTGTTTTTGAGGTTAGTAGTGG | | ATAAAGGGTTATTCAACAACA | |
| | | GGATAACTTTTACGTCCAATTGTTGA | | ACGAGTGTCTGAGACGTTACA | | TAAAAATGGTACAATATTCTA | |
| | | GGAAGGGTTTAGACAAGAAGAAATT | | ACCATCTAGAAGACGAAATCG | | GGTGTGTTTTACTATTCTTTGC | |
| | | GAAAGAAGGATCTACACATCCTGTT | | ATAAATGGCCAGATATGACGA | | TTGAAATCCCAAAAAGGTGA | |
| | | GTAGAAGATGTTTGGAATTCCCCCAT | | AGGATAACTTTTACGTCCAATT | | CCCTTCAAGTTCACTTGATAC | |
| | | CGTCCAAGAAACATTCCTATCCCAGC | | GTTGAGGAAGGGTTTAGACAA | | ACTAACTTCAACAAATGGGG | |
| | | AAGGAGAAGGAAATAATCCCATAAA | | GAAGAAATTGAAAGAAGGATC | | ATATTTCTGCCTTCACCTTTTG | |
| | | GAGACATCTCATGGATTTCAATACCA | | TACACATCCTGTTGTAGAAGA | | CACAACAGACTCTATGAATTT | |
| | | TCACCTACGCCGCCAAACAACTAAA | | TGTTTGGAATTCCCCCATCGTC | | ATTACACATCAAATTCAATT | |
| | | AACTTGCTTCGAAACAAACCTACGCA | | CAAGAAACATTCCTATCCCAG | | | |
| | | CCCATTTCCGGACACGACAACAGAG | | CAAGGAGAAGGAAATAATCCC | | | |
| | | GGCCATATCTGGATGGTTAGCTGAAA | | ATAAAGAGACATCTCATGGAT | | | |
| | | ACGGGTTCGATAAAAAGTATACGAA | | TTCAATACCATCACCTACGCCG | | | |
| | | ACTCGTACAACACTGGATAATTGGAT | | CCAAACAACTAAAAACTTGCT | | | |
| | | GTACCTACAAGAGTGATTGGGTGGA | | TCGAAACAAACCTACGCACCC | | | |
| | | CAGTGGTGATTTGGAAAGGGTAAAA | | ATTTCCGGACACGACAACAGA | | | |
| | | GAAGGAACGAAAAATTTCGTGACTC | | GGGCCATATCTGGATGGTTAG | | | |
| | | TTCATAGGAAACATTTATGTGTTATT | | CTGAAAACGGGTTCGATAAAA | | | |
| | | AGTGATAAGAAGAATGGTACAATTT | | AGTATACGAAACTCGTACAAC | | | |
| | | CCTATTCACCTGAAGAGAAATATCCG | | ACTGGATAATTGGATGTACCT | | | |
| | | ATACCCTCAATACTAAATTATTACAA | | ACAAGAGTGATTGGGTGGACA | | | |
| | | GTTTCTACAAACAGAGTATCCACAAA | | GTGGTGATTTGGAAAGGGTAA | | | |
| | | ACAAGAAAATACAGAAAATGATAGT | | AAGAAGGAACGAAAAATTTCG | | | |
| | | TGTCCCAAAACACAAACTAAAGATA | | TGACTCTTCATAGGAAACATTT | | | |
| | | CACTATTGTACGTTTGACCAAACGAC | | ATGTGTTATTAGTGATAAGAA | | | |
| | | CATTCAAGGAATTTGTAAAGATTTGG | | GAATGGTACAATTTCCTATTCA | | | |
| | | GAGTGTGGAAGGATATGGAAGAACG | | CCTGAAGAGAAATATCCGATA | | | |
| | | ACACAAACAATCAGAAGATATACTT | | CCCTCAATACTAAATTATTACA | | | |
| | | TACAAGCAAGGATGGTACCTATTATT | | AGTTTCTACAAACAGAGTATC | | | |
| | | CGACGTTAAAAAGATTAAGAAATTG | | CACAAAACAAGAAAATACAGA | | | |
| | | CGTCCAAACTGGAACTTTCACTCTAT | | AAATGATAGTTGTCCCAAAAC | | | |
| | | CCAGACGGACGGCGAAGGCGTCTCT | | ACAAACTAAAGATACACTATT | | | |
| | | GTACTATTTTCCAGAGAAGTGGAAG | | GTACGTTTGACCAAACGACCA | | | |
| | | AAGTAGAGACTGTTTCCAAGAAAAG | | TTCAAGGAATTTGTAAAGATTT | | | |
| | | TAAGAAAAATAAAAAACCTAGAGGA | | GGGAGTGTGGAAGGATATGGA | | | |
| | | GATGAGGATAGGAGAAATTACCCGC | | AGAACGACACAAACAATCAGA | | | |
| | | CCACTAATGCCAAGTACGTAGTGGGT | | AGATATACTTTACAAGCAAGG | | | |
| | | GTAGATCCGGGAAGAACTAATGTCG | | ATGGTACCTATTATTCGACGTT | | | |
| | | TTTCCTGTTCGGTATTTGATACCCGTC | | AAAAAGATTAAGAAATTGCGT | | | |
| | | AAAAAAGGGTAGTGAGAAAACACAG | | CCAAACTGGAACTTTCACTCTA | | | |
| | | AATGACTGCGAAACAATACTATCAA | | TCCAGACGGACGGCGAAGGCG | | | |
| | | GAATCTTGGATGACAGATAGAAGAA | | TCTCTGTACTATTTTCCAGAGA | | | |
| | | AGGCAAACGAAACGTACAAGAAGAA | | AGTGGAAGAAGTAGAGACTGT | | | |
| | | CAATAAAGAGTACAAAGAGGCGTTA | | TTCCAAGAAAAGTAAGAAAAA | | | |
| | | GAGGAAATAACTAGGTACGATAATG | | TAAAAAACCTAGAGGAGATGA | | | |
| | | GCGAAGAAATTATAAATGATGGTAA | | GGATAGGAGAAATTACCCGCC | | | |
| | | CGGTGATACTTCTACACCCACTAAAA | | CACTAATGCCAAGTACGTAGT | | | |
| | | AATTCGAAGCTTACTTGAAGGTAGTG | | GGGTGTAGATCCGGGAAGAAC | | | |
| | | AACGAGCACTACAGGTTACTGTGGA | | TAATGTCGTTTCCTGTTCGGTA | | | |
| | | ACGAAAAGGGAAAGAAAAAGTACA | | TTTGATACCCGTCAAAAAAGG | | | |
| | | GGAAAAATGCCATGAAAGTATACTC | | GTAGTGAGAAAACACAGAATG | | | |
| | | TAGAAAACAAAAGTGCATATCTAAC | | ACTGCGAAACAATACTATCAA | | | |
| | | TTTATAGATGAATTAATCCCTAAAAG | | GAATCTTGGATGACAGATAGA | | | |
| | | GGATAAAATTGAAGATTACCACATT | | AGAAAGGCAAACGAAACGTAC | | | |
| | | GCTTTTGGGGATGCGAAATTTGCCTG | | AAGAAGAACAATAAAGAGTAC | | | |
| | | CACGGGAAGAGGTGAGCAATACGCA | | AAAGAGGCGTTAGAGGAAATA | | | |
| | | TCACCTGCCAGGATTTTCGCCAAGAA | | ACTAGGTACGATAATGGCGAA | | | |
| | | GATAAAGGAAAGAGTCGGAGGTGAT | | GAAATTATAAATGATGGTAAC | | | |
| | | AAGAGGTTTACTTTCGTGGACGAGA | | GGTGATACTTCTACACCCACTA | | | |
| | | AATATACGTCAAAAGTATGCCATCGC | | AAAAATTCGAAGCTTACTTGA | | | |
| | | TGCAATCAACCTTTAAATATGCTGGA | | AGGTAGTGAACGAGCACTACA | | | |
| | | GAAGGATTGTTTTTCACCGAATAAGA | | GGTTACTGTGGAACGAAAAGG | | | |
| | | AAAGAAAACCGCCGACAATAGTAAC | | GAAAGAAAAAGTACAGGAAA | | | |
| | | AACCACAACAACAACAACAACAGAA | | AATGCCATGAAAGTATACTCT | | | |
| | | GAAGACGAAGAAAATGGAAAATGGA | | AGAAAACAAAAGTGCATATCT | | | |
| | | AGAAGGCTACACCTCTCAGAGAAAA | | AACTTTATAGATGAATTAATCC | | | |
| | | TAGAGATACCAGAAGATGCTCGTCC | | CTAAAAGGGATAAAATTGAAG | | | |
| | | GAAAAGACGCAATTCGGTTACAGTT | | ATTACCACATTGCTTTTGGGGA | | | |
| | | CAAACCGAAAAGTATCGACAGGAGA | | TGCGAAATTTGCCTGCACGGG | | | |
| | | TATCTCTATGGAAACGCCAGTACCTT | | AAGAGGTGAGCAATACGCATC | | | |
| | | CTTCCACTTCCTCTTCCTTTTGTACTC | | ACCTGCCAGGATTTTCGCCAA | | | |
| | | CTACTTCCATTACATGTGTCTTGGGA | | GAAGATAAAGGAAAGAGTCG | | | |
| | | GGAAAATTCGTCGACAGGGACTTCA | | GAGGTGATAAGAGGTTTACTT | | | |
| | | ATGCAAGCACCAATATTGTTCATAAA | | TCGTGGACGAGAAATATACGT | | | |
| | | TTTCTAGGGTTTTGGGATAAAAAGTT | | CAAAAGTATGCCATCGCTGCA | | | |
| | | AATGGAAAAGAAAGACAAGATGCCG | | ATCAACCTTTAAATATGCTGG | | | |
| | | TTGAAGTATCACTTTATTCGAGTTGC | | AGAAGGATTGTTTTTCACCGA | | | |
| | | CTGAGAATAGGATGTAAAAAACTCG | | ATAAGAAAAGAAAACCGCCGA | | | |
| | | TGTCACTAGTGAAATAGTGGCGTAGC | | CAATAGTAACAACCACAACAA | | | |
| | | CCTGAAGGATGAGAAGTATGCTATTT | | CAACAACAACAGAAGAAGAC | | | |
| | | TCGACCTTACGGTCATTGTGGTATAT | | GAAGAAAATGGAAAATGGAA | | | |
| | | CAACTCTTGCCCGATAAAGGGTTATT | | GAAGGCTACACCTCTCAGAGA | | | |
| | | CAACAACATAAAAATGGTACAATAT | | AAATAGAGATACCAGAAGATG | | | |
| | | TCTAGGTGTGTTTTACTATTCTTTGCT | | CTCGTCCGAAAAGACGCAATT | | | |
| | | TGAAATCCCAAAAAGGTGACCCTTC | | CGGTTACAGTTCAAACCGAAA | | | |
| | | AAGTTCACTTGATACACTAACTTCAA | | AGTATCGACAGGAGATATCTC | | | |
| | | CAAATGGGGATATTTCTGCCTTCACC | | TATGGAAACGCCAGTACCTTC | | | |
| | | TTTTGCACAACAGACTCTATGAATTT | | TTCCACTTCCTCTTCCTTTTGTA | | | |
| | | ATTACACATCAAATTCAATT | | CTCCTACTTCCATTACATGTGT | | | |
| | | | | CTTGGGAGGAAAATTCGTCGA | | | |
| | | | | CAGGGACTTCAATGCAAGCAC | | | |
| | | | | CAATATTGTTCATAAATTTCTA | | | |
| | | | | GGGTTTTGGGATAAAAAGTTA | | | |
| | | | | ATGGAAAAGAAAGACAAGAT | | | |
| | | | | GCCGTTGAAGTATCACTTTATT | | | |
| | | | | CGAGTTGCCTGA | | | |
| 95 | YP_ | ATGCCTGTGTGCAAATCTTGTGGTGG | 216 | ATGCCTGTGTGCAAATCTTGTG | 247 | CTCAAGCCTGTTCCACGTGTA | 278 |
| | 001649035.1 | ACTAGACCATCAGCGTAGATCGTCCA | | GTGGACTAGACCATCAGCGTA | | ACCAGCAGACTGGAGAGTTT | |
| | | CGAAATGCGCCAACTTTTTGCCTCAA | | GATCGTCCACGAAATGCGCCA | | AGTAAAATTGTAATGGATCAC | |
| | | GTGAGAAAACGAAAACTGGAACCGC | | ACTTTTTGCCTCAAGTGAGAA | | AAGAAGCGAGTGGGAGTTTG | |
| | | AACAAGTGAACTTGGATTTGCAAGT | | AACGAAAACTGGAACCGCAAC | | CCCAAACTGCGCCAGCCGCTT | |
| | | GTACGTCGTGAAACGAGGTTTACAG | | AAGTGAACTTGGATTTGCAAG | | CGATCGTGACCAGAACGCTGC | |
| | | AGCGCTTTGTGTCCGTTGTTGAGCGA | | TGTACGTCGTGAAACGAGGTT | | CAAAAACATTTTAATTAACTT | |
| | | CGACGACCGCAATAGACTACTTTACG | | TACAGAGCGCTTTGTGTCCGTT | | TATGAGGTCTACCGTCTGTAG | |
| | | AGATCAGGCGAGACGTACGAGATTT | | GTTGAGCGACGACGACCGCAA | | GCCAAGATGCTCTTACACTAA | |
| | | GTCTAGGGTCTACATCGAATTGGGCA | | TAGACTACTTTACGAGATCAG | | AAGGGTATCGTAGCTTCCTGA | |
| | | TTCTGTTCAACTATTACATGAAACGG | | GCGAGACGTACGAGATTTGTC | | AACGTAAATGTTGTAAAATCA | |
| | | TGCGACACGTTGCCCGACAAACCGG | | TAGGGTCTACATCGAATTGGG | | AACGCAATAAGGTTCAATTTC | |
| | | ACGTTTTAAATTTTTTCTACGCCATG | | CATTCTGTTCAACTATTACATG | | AAATTGGCATTTATTTGCGTT | |
| | | AAAGGCAAAGGACCGTACGCTGCGT | | AAACGGTGCGACACGTTGCCC | | TGGTTCTCAGGAGGTTATCTA | |
| | | GCTACAAACATTTGGCGGGAGACCA | | GACAAACCGGACGTTTTAAAT | | CACGTCGACAATTTTGCCATG | |
| | | AGTGTACGACGGTAAATTGAGAAGT | | TTTTTCTACGCCATGAAAGGCA | | ACATACTTGGTTTCATGTCGG | |
| | | TTTGTGGTGCAAGAACTGGCCAAAC | | AAGGACCGTACGCTGCGTGCT | | AACTCTCGCTCACACCCAAAC | |
| | | AGTACGAGACACTAATCCATACCAA | | ACAAACATTTGGCGGGAGACC | | AATGCACAGCACAACAGATT | |
| | | TATCGAGACTCATGCTTATTCGAGAC | | AAGTGTACGACGGTAAATTGA | | GTTACGCTGCTGGTGTGTACG | |
| | | TAGCTCGATATTTTGGAGTGAAAAAG | | GAAGTTTTGTGGTGCAAGAAC | | AGCGCCCTCAAAATCATAATC | |
| | | AACGACCATGGCTTATTCTTAGCCTA | | TGGCCAAACAGTACGAGACAC | | TTTGTCGTCAATCAATTTACA | |
| | | CTACAAGAAAGATTTTAGTCAAGCC | | TAATCCATACCAATATCGAGA | | CACAAAGCGCAGATACTGCG | |
| | | ACCGAGGATATGATTAGAGTGTGCC | | CTCATGCTTATTCGAGACTAGC | | ACTCGTAAGGCTCGGGTAGAT | |
| | | AAATTATTAACGTCGACAGCTGCAA | | TCGATATTTTGGAGTGAAAAA | | TGCGAGATTTCATAAAGTCC | |
| | | GAAAAATTGGTGGTCTACTGTCCCAC | | GAACGACCATGGCTTATTCTTA | | | |
| | | TATGGCTGAAGATCCAACAGGATTTG | | GCCTACTACAAGAAAGATTTT | | | |
| | | TTCGACAGAGATAATGTAGAATCTTT | | AGTCAAGCCACCGAGGATATG | | | |
| | | CAGGCTATTTCCTCAACCTTCGTATG | | ATTAGAGTGTGCCAAATTATT | | | |
| | | GATTAAAACACTTGACATACACATCT | | AACGTCGACAGCTGCAAGAAA | | | |
| | | CGGGGATGGCACGAATTATTGAGAA | | AATTGGTGGTCTACTGTCCCAC | | | |
| | | GAGTCAAACCCGACTCTGTTCCTAGT | | TATGGCTGAAGATCCAACAGG | | | |
| | | CACTGGCCTAATATTCAAGATCACAT | | ATTTGTTCGACAGAGATAATG | | | |
| | | CAAAGAACTGTGGGCTCCTTGGCTCA | | TAGAATCTTTCAGGCTATTTCC | | | |
| | | AATCTACAAAAAACTTTGGCTGTTGT | | TCAACCTTCGTATGGATTAAA | | | |
| | | CTACAGACAGACGGTGTGAGTGTCA | | ACACTTGACATACACATCTCG | | | |
| | | GTCTTTCTATGAATCGTTGGATTAAG | | GGGATGGCACGAATTATTGAG | | | |
| | | AAGGTGACAAAGAATGGTATTACTA | | AAGAGTCAAACCCGACTCTGT | | | |
| | | AAGAGGTGGTAATTAAGAAATGGAA | | TCCTAGTCACTGGCCTAATATT | | | |
| | | GCCTAGCTACGACAAGCTAATAGCG | | CAAGATCACATCAAAGAACTG | | | |
| | | GTGGATCCTGGATCAAGAGTTCCCGT | | TGGGCTCCTTGGCTCAAATCTA | | | |
| | | TGCAGCTTTTAACTCTCATGGATTTT | | CAAAAAACTTTGGCTGTTGTCT | | | |
| | | ACAAACGTGTCACCAGTAAATTTGTT | | ACAGACAGACGGTGTGAGTGT | | | |
| | | AAATCACACAGTCTAGATTGGAAAA | | CAGTCTTTCTATGAATCGTTGG | | | |
| | | GGGAGAGGGTTAGAACTAAATTGAC | | ATTAAGAAGGTGACAAAGAAT | | | |
| | | TAAATCTAGTGAAACGGCTGAGGCT | | GGTATTACTAAAGAGGTGGTA | | | |
| | | AAAGATAGAGAACGAGTACAAACGA | | ATTAAGAAATGGAAGCCTAGC | | | |
| | | CTTGCAATATTGTGTTGTCTTCCAAG | | TACGACAAGCTAATAGCGGTG | | | |
| | | AATACAAGTCACGTGGTAGAGTATA | | GATCCTGGATCAAGAGTTCCC | | | |
| | | CAGTGTTTCGACTTAAATGGTTCGAC | | GTTGCAGCTTTTAACTCTCATG | | | |
| | | GTCAGACAGAAACCCTACGAAACAC | | GATTTTACAAACGTGTCACCA | | | |
| | | ACTCTAAACTCACCAGACTCATGTTT | | GTAAATTTGTTAAATCACACA | | | |
| | | GATAAATACATAAACACTGCCAAGT | | GTCTAGATTGGAAAAGGGAGA | | | |
| | | TGCAAGAAAAGATTGTAAAAGAAGT | | GGGTTAGAACTAAATTGACTA | | | |
| | | GTTTGACGGAGGAGGTGTGGAAAAA | | AATCTAGTGAAACGGCTGAGG | | | |
| | | ACTTTAGTGTTGTATGGTGCCGGATC | | CTAAAGATAGAGAACGAGTAC | | | |
| | | AAAGTTTATGAACGCGGCTCAATTCT | | AAACGACTTGCAATATTGTGTT | | | |
| | | CTGGTCGAAAATTCAAGCACGATGCT | | GTCTTCCAAGAATACAAGTCA | | | |
| | | CTATTGAAAAAGCTTAGGCAGAAAA | | CGTGGTAGAGTATACAGTGTT | | | |
| | | GAAATATTCATGTAGAAATTGTAGAC | | TCGACTTAAATGGTTCGACGTC | | | |
| | | GAATCGTACACTTCTCAAGCCTGTTC | | AGACAGAAACCCTACGAAACA | | | |
| | | CACGTGTAACCAGCAGACTGGAGAG | | CACTCTAAACTCACCAGACTC | | | |
| | | TTTAGTAAAATTGTAATGGATCACAA | | ATGTTTGATAAATACATAAAC | | | |
| | | GAAGCGAGTGGGAGTTTGCCCAAAC | | ACTGCCAAGTTGCAAGAAAAG | | | |
| | | TGCGCCAGCCGCTTCGATCGTGACCA | | ATTGTAAAAGAAGTGTTTGAC | | | |
| | | GAACGCTGCCAAAAACATTTTAATTA | | GGAGGAGGTGTGGAAAAAACT | | | |
| | | ACTTTATGAGGTCTACCGTCTGTAGG | | TTAGTGTTGTATGGTGCCGGAT | | | |
| | | CCAAGATGCTCTTACACTAAAAGGGT | | CAAAGTTTATGAACGCGGCTC | | | |
| | | ATCGTAGCTTCCTGAAACGTAAATGT | | AATTCTCTGGTCGAAAATTCA | | | |
| | | TGTAAAATCAAACGCAATAAGGTTC | | AGCACGATGCTCTATTGAAAA | | | |
| | | AATTTCAAATTGGCATTTATTTGCGT | | AGCTTAGGCAGAAAAGAAATA | | | |
| | | TTGGTTCTCAGGAGGTTATCTACACG | | TTCATGTAGAAATTGTAGACG | | | |
| | | TCGACAATTTTGCCATGACATACTTG | | AATCGTACACTTCTCAAGCCTG | | | |
| | | GTTTCATGTCGGAACTCTCGCTCACA | | TTCCACGTGTAACCAGCAGAC | | | |
| | | CCCAAACAATGCACAGCACAACAGA | | TGGAGAGTTTAGTAAAATTGT | | | |
| | | TTGTTACGCTGCTGGTGTGTACGAGC | | AATGGATCACAAGAAGCGAGT | | | |
| | | GCCCTCAAAATCATAATCTTTGTCGT | | GGGAGTTTGCCCAAACTGCGC | | | |
| | | CAATCAATTTACACACAAAGCGCAG | | CAGCCGCTTCGATCGTGACCA | | | |
| | | ATACTGCGACTCGTAAGGCTCGGGTA | | GAACGCTGCCAAAAACATTTT | | | |
| | | GATTGCGAGATTTCATAAAGTCC | | AATTAACTTTATGAGGTCTACC | | | |
| | | | | GTCTGTAGGCCAAGATGCTCTT | | | |
| | | | | ACACTAAAAGGGTATCGTAG | | | |
| 97 | YP_ | ATGCCTCTGTGTAAATCTTGCGGTGG | 217 | ATGCCTCTGTGTAAATCTTGCG | 248 | CTTCACAAGCCTGCTCAGCGT | 279 |
| | 003422378.1 | ACTAGACCATCAGCGTAGAACTTCCA | | GTGGACTAGACCATCAGCGTA | | GCAACCATAAAAGTGGAGTG | |
| | | CAAAGTGCGCCAATTATTTGCCTCAA | | GAACTTCCACAAAGTGCGCCA | | TTTAGCAAAATAAGCATGGAT | |
| | | GTGAGAAAACGAAAACTGGAACCGC | | ATTATTTGCCTCAAGTGAGAA | | CACAAGAGGAGAAGGGGAGT | |
| | | ATCAAGAGAATCTAGAGTTACAAAT | | AACGAAAACTGGAACCGCATC | | TTGCCCAAACTGCTCCAGCTC | |
| | | CTATGTTGTAAAGAGAGGACTACAA | | AAGAGAATCTAGAGTTACAAA | | GTTCGATCGTGACCAGAATGC | |
| | | AGTGCTCTATGTCCTCTGTTAAGTGA | | TCTATGTTGTAAAGAGAGGAC | | TGCCAAGAATATTTTAATCAA | |
| | | CGAGGATAGGAACAAGTTGTTGTAC | | TACAAAGTGCTCTATGTCCTCT | | CTATTTAAGGTCTGCCGTCTG | |
| | | GAGATCAGACGGGATGTCAGGGATT | | GTTAAGTGACGAGGATAGGAA | | CAGGCCAAGATGCTCTATAAG | |
| | | TGTCTAGAGTCTACATTGAATTGGGT | | CAAGTTGTTGTACGAGATCAG | | AAGGGTATCGTAGCTTCCTAA | |
| | | ATTGTGATGAATTACTACATGAAGCG | | ACGGGATGTCAGGGATTTGTC | | AACAAAATTGTTGTGAAATCA | |
| | | GTGTGATGTGTTACCAGACAAACCA | | TAGAGTCTACATTGAATTGGG | | AACGCAATAAGGTTCAAATTC | |
| | | GACGTACTCAACTTTTTTTACGCCAT | | TATTGTGATGAATTACTACATG | | AATTTGGCATTATTTGTGTTT | |
| | | GAAAGGCAAGGGACCTTACGCTGCG | | AAGCGGTGTGATGTGTTACCA | | GGTTCTTAGGAGGTTATATTT | |
| | | TGCTACAAACACTTGGCGGGAGACA | | GACAAACCAGACGTACTCAAC | | ACCACATAAAATATACAACTT | |
| | | GAGTGTACGATGGTAAACTCAGATC | | TTTTTTTACGCCATGAAAGGCA | | AACCAATTGGTTTTGATTAAA | |
| | | GTTTGTAGTGCAAGAACTAGCCAAA | | AGGGACCTTACGCTGCGTGCT | | ATCTGCACTCGACTCGCTCAT | |
| | | CAGTATGAAACGTTGATTCACACTAA | | ACAAACACTTGGCGGGAGACA | | TTTTGCACCATAACGGTACAA | |
| | | TATAGAGACACATGCTTATTCAAGAC | | GAGTGTACGATGGTAAACTCA | | TAGAAATAAAATTTTATTGAC | |
| | | TGGCAAAATATTTTGGAGTAAAAAA | | GATCGTTTGTAGTGCAAGAAC | | AAATAAGTTTGCTGTAAATTT | |
| | | GAACAACGATGGCTTGTTCCAAGCAT | | TAGCCAAACAGTATGAAACGT | | GTTCATGTACTTAAAGGTGGT | |
| | | ATTACAAGAAAGATTTTAGCCATGCC | | TGATTCACACTAATATAGAGA | | CTCTATATAACATGGTAAATA | |
| | | ACGGAAGAAATGAAACAAATTTGTC | | CACATGCTTATTCAAGACTGG | | AGCTGAACTGTATTTTTTTAA | |
| | | AAGTTATTAACGCAGAGAGCTGCAA | | CAAAATATTTTGGAGTAAAAA | | GAGATATCTTTCAATTGAA | |
| | | GAAGGATTGGTGGAACACCATACCT | | AGAACAACGATGGCTTGTTCC | | | |
| | | CTATGGCTAAAAATCCAACAGGAAT | | AAGCATATTACAAGAAAGATT | | | |
| | | TGTTTGACAGAGACAAGGTAGAGTC | | TTAGCCATGCCACGGAAGAAA | | | |
| | | TTTTAGGCTATTCCCTCAACCTAGCT | | TGAAACAAATTTGTCAAGTTA | | | |
| | | ATGGCTTGAAACATTTGACCTATACT | | TTAACGCAGAGAGCTGCAAGA | | | |
| | | TCTAGAGGATGGCACGAGCTACTCA | | AGGATTGGTGGAACACCATAC | | | |
| | | GAAGGGTTAAACCTGACACAACACC | | CTCTATGGCTAAAAATCCAAC | | | |
| | | CAGTAGTTGGATGTCTATTGATGATC | | AGGAATTGTTTGACAGAGACA | | | |
| | | ATATAAAGGAATTATGGGAACCTTG | | AGGTAGAGTCTTTTAGGCTATT | | | |
| | | GCTAAAGTCTACCAAAAACTTTGGAT | | CCCTCAACCTAGCTATGGCTTG | | | |
| | | GCTGTCTTCAAACGGATGGGATTAGT | | AAACATTTGACCTATACTTCTA | | | |
| | | GTGAGTCTATCCATGAATCGTTGGGT | | GAGGATGGCACGAGCTACTCA | | | |
| | | CAAGAAAGTAACAAAGAACGGTGAA | | GAAGGGTTAAACCTGACACAA | | | |
| | | ACAAAAGAAGTAGTGATTAAAAAGT | | CACCCAGTAGTTGGATGTCTAT | | | |
| | | GGAAGCCGAGCTACGACAAACTGAT | | TGATGATCATATAAAGGAATT | | | |
| | | AGCGGTGGATCCTGGATCAAGAATA | | ATGGGAACCTTGGCTAAAGTC | | | |
| | | CCTGTAGCTGCCTACAATTCTAACGG | | TACCAAAAACTTTGGATGCTG | | | |
| | | TGTATATCAAAGAATTACTAGTAAAT | | TCTTCAAACGGATGGGATTAG | | | |
| | | ACGTCAAGTCACACAGTCTAGAATG | | TGTGAGTCTATCCATGAATCGT | | | |
| | | GAAAAGAGATAGAGTGAGAAATAAA | | TGGGTCAAGAAAGTAACAAAG | | | |
| | | CTGACTAAATCTAGCGAAATGGCTG | | AACGGTGAAACAAAAGAAGTA | | | |
| | | AAGCTAAAGATAGAGAGCGAGTACA | | GTGATTAAAAAGTGGAAGCCG | | | |
| | | AGCGGCTTGCAGTATTGTATTGTCTT | | AGCTACGACAAACTGATAGCG | | | |
| | | CGAAAAATACAAGCCACGTGGTAGA | | GTGGATCCTGGATCAAGAATA | | | |
| | | GTATACAGTGTTTCGTCTAAAATGGT | | CCTGTAGCTGCCTACAATTCTA | | | |
| | | TCAACGTCAGACAACAACCCTATGA | | ACGGTGTATATCAAAGAATTA | | | |
| | | AATACACTCTAAGCTCACCAGATTGA | | CTAGTAAATACGTCAAGTCAC | | | |
| | | AATTCGACAAGTATATAAACACTGG | | ACAGTCTAGAATGGAAAAGAG | | | |
| | | AAAAGTACAAGAAAAGATTGTTAAA | | ATAGAGTGAGAAATAAACTGA | | | |
| | | CAAGTGTTTGACGGAGGAGGTGTGG | | CTAAATCTAGCGAAATGGCTG | | | |
| | | AGAAAACTCTGGTGTTATATGGTGCA | | AAGCTAAAGATAGAGAGCGAG | | | |
| | | GGATCGAACTTTATGAACACTGCTCA | | TACAAGCGGCTTGCAGTATTG | | | |
| | | GTTTAGCGGTCGTAAATTTAAACACA | | TATTGTCTTCGAAAAATACAA | | | |
| | | ACGCTCTGTTAAAGAAACTAAGGCA | | GCCACGTGGTAGAGTATACAG | | | |
| | | GAAGAGAAACATACATGTTGAAATT | | TGTTTCGTCTAAAATGGTTCAA | | | |
| | | GTAGACGAATCGTATACTTCACAAGC | | CGTCAGACAACAACCCTATGA | | | |
| | | CTGCTCAGCGTGCAACCATAAAAGT | | AATACACTCTAAGCTCACCAG | | | |
| | | GGAGTGTTTAGCAAAATAAGCATGG | | ATTGAAATTCGACAAGTATAT | | | |
| | | ATCACAAGAGGAGAAGGGGAGTTTG | | AAACACTGGAAAAGTACAAGA | | | |
| | | CCCAAACTGCTCCAGCTCGTTCGATC | | AAAGATTGTTAAACAAGTGTT | | | |
| | | GTGACCAGAATGCTGCCAAGAATAT | | TGACGGAGGAGGTGTGGAGAA | | | |
| | | TTTAATCAACTATTTAAGGTCTGCCG | | AACTCTGGTGTTATATGGTGCA | | | |
| | | TCTGCAGGCCAAGATGCTCTATAAGA | | GGATCGAACTTTATGAACACT | | | |
| | | AGGGTATCGTAGCTTCCTAAAACAA | | GCTCAGTTTAGCGGTCGTAAA | | | |
| | | AATTGTTGTGAAATCAAACGCAATA | | TTTAAACACAACGCTCTGTTAA | | | |
| | | AGGTTCAAATTCAATTTGGCATTATT | | AGAAACTAAGGCAGAAGAGA | | | |
| | | TGTGTTTGGTTCTTAGGAGGTTATAT | | AACATACATGTTGAAATTGTA | | | |
| | | TTACCACATAAAATATACAACTTAAC | | GACGAATCGTATACTTCACAA | | | |
| | | CAATTGGTTTTGATTAAAATCTGCAC | | GCCTGCTCAGCGTGCAACCAT | | | |
| | | TCGACTCGCTCATTTTTGCACCATAA | | AAAAGTGGAGTGTTTAGCAAA | | | |
| | | CGGTACAATAGAAATAAAATTTTATT | | ATAAGCATGGATCACAAGAGG | | | |
| | | GACAAATAAGTTTGCTGTAAATTTGT | | AGAAGGGGAGTTTGCCCAAAC | | | |
| | | TCATGTACTTAAAGGTGGTCTCTATA | | TGCTCCAGCTCGTTCGATCGTG | | | |
| | | TAACATGGTAAATAAGCTGAACTGT | | ACCAGAATGCTGCCAAGAATA | | | |
| | | ATTTTTTTAAGAGATATCTTTCAATT | | TTTTAATCAACTATTTAAGGTC | | | |
| | | GAA | | TGCCGTCTGCAGGCCAAGATG | | | |
| | | | | CTCTATAAGAAGGGTATCGTA | | | |
| | | | | G | | | |
| 98 | YP_762432.1 | ATGCCGCTGTCGTGCAAGTCGTGCGG | 218 | ATGCCGCTGTCGTGCAAGTCG | 249 | GACTTCGTCGCGGCGTCTGTC | 280 |
| | | CGGCGAAGGTCACACTCGCCGCAGC | | TGCGGCGGCGAAGGTCACACT | | CAGACTGCAACGCCGACGTC | |
| | | TCGAAATCGTGTGCTAACTATCTGCC | | CGCCGCAGCTCGAAATCGTGT | | GAGAGGGACTACAACGCCGC | |
| | | ACCGATTCGGCAACGAAGAAACAGC | | GCTAACTATCTGCCACCGATTC | | CAAAAATATACTAGTTAATTA | |
| | | GGCGACGTAGACCACTACACACAGG | | GGCAACGAAGAAACAGCGGC | | TCAAAGGTCCGCCGTCTGCAG | |
| | | ACTTTGACGTGGGTGTTGTGAAGCAG | | GACGTAGACCACTACACACAG | | GCCGAGCCCGAGCTGTGACTC | |
| | | GGTCTCGATGGTACAATCTGTCCACT | | GACTTTGACGTGGGTGTTGTG | | GGGCGTAGCCACCGACACGG | |
| | | CCTACGAGAAGACGTGCGCCAACGT | | AAGCAGGGTCTCGATGGTACA | | ACCCGCACTCCATACGGTCCA | |
| | | CTGAAGGTGGAGATCAGATCCGATG | | ATCTGTCCACTCCTACGAGAA | | TTTTCCAATGCGCCGTATCGG | |
| | | TTGTGGAGCTGTCTAAACTTTACATA | | GACGTGCGCCAACGTCTGAAG | | AGCCCGGGTCTTAGGAGGTTA | |
| | | TTGCTCGGTGTGTTCGTAAATCACTG | | GTGGAGATCAGATCCGATGTT | | CCGTGCACCCTTCTCACTCGG | |
| | | TTGGAACGCATACCCCGACGACGAA | | GTGGAGCTGTCTAAACTTTAC | | ACGTGGGGTGATTGCGAGAC | |
| | | TCGAACAGCGTTCGCACGTTTCAGGA | | ATATTGCTCGGTGTGTTCGTAA | | GTGCGGCCAACGCACGAATC | |
| | | CATGATGAACTACGTGTATGCGATCA | | ATCACTGTTGGAACGCATACC | | GTGCTACGACGTGTCGACGAC | |
| | | GAGGCAAGGGACCACACGCGAAGAA | | CCGACGACGAATCGAACAGCG | | AAGTGTGCCACCGTTGCCATC | |
| | | ATTCGAAGATATGGTGCGCTATCACG | | TTCGCACGTTTCAGGACATGAT | | GTTATCACGAACATTGTCAGT | |
| | | GCAATATACGTCGCTACGACGGACG | | GAACTACGTGTATGCGATCAG | | ACTGTGAACGCAGTAGTGAG | |
| | | ACTTCGTTCATTCATCGTTCAAGAAG | | AGGCAAGGGACCACACGCGAA | | ACGTCAGTCGACTCGTAAACA | |
| | | TGGCTAAAACCTATTGGACTGTGCTG | | GAAATTCGAAGATATGGTGCG | | TGATACTGTTCGTTACGTGTT | |
| | | AAGACGAACATCACGACGCACGCCT | | CTATCACGGCAATATACGTCG | | TGTTCGTCACCACCGTTTACG | |
| | | ACTCGCGCCTCGCCCGTTACCTCGGT | | CTACGACGGACGACTTCGTTC | | GCGATTGCTCGACTACGACGC | |
| | | GTGAAGAGAAACGATCCGACCCTTTT | | ATTCATCGTTCAAGAAGTGGC | | TGTCGTTTGTCCGAAAAACCG | |
| | | CGCAGCCTACTATCACAAGGAGTTCG | | TAAAACCTATTGGACTGTGCT | | GAAACTGCTTCGATTACGAAC | |
| | | GTGACGACGATTCGATATCGAGGGT | | GAAGACGAACATCACGACGCA | | AGGGATCGGTGAGCCATCCC | |
| | | GTGTAGATTCATCAACGTATCGAGCG | | CGCCTACTCGCGCCTCGCCCGT | | ACG | |
| | | ACTGGCATTCGACCATACCGATGTGG | | TACCTCGGTGTGAAGAGAAAC | | | |
| | | ATCGAAATACAGCGAGACATGTATG | | GATCCGACCCTTTTCGCAGCCT | | | |
| | | CGCGCGGCGAACAATCATTCGTCATC | | ACTATCACAAGGAGTTCGGTG | | | |
| | | TTCCCGCAGCCGAGCCATGGTTTGAA | | ACGACGATTCGATATCGAGGG | | | |
| | | ACACGTCACGTACACCAGCAAAGGA | | TGTGTAGATTCATCAACGTATC | | | |
| | | TGGCACGAACTGTTGAGACGCGTCG | | GAGCGACTGGCATTCGACCAT | | | |
| | | ATCCATCGAACGTCAAATCGAATTGG | | ACCGATGTGGATCGAAATACA | | | |
| | | CCTTCCATAACGGATCACAAGAAAG | | GCGAGACATGTATGCGCGCGG | | | |
| | | AGTTGTGGGCGCCATACCTAGACAC | | CGAACAATCATTCGTCATCTTC | | | |
| | | GTGTGTAGTTGACATGAAGAAGTTTG | | CCGCAGCCGAGCCATGGTTTG | | | |
| | | GATGTTGCATTCAAACGGACGGTGTA | | AAACACGTCACGTACACCAGC | | | |
| | | GCCGTGTCGATATCGATGAATAGACC | | AAAGGATGGCACGAACTGTTG | | | |
| | | GAAGAGGACGTCCGTCCGTCGAAAG | | AGACGCGTCGATCCATCGAAC | | | |
| | | GCAGCTCTACCCAAATCGTATACAAT | | GTCAAATCGAATTGGCCTTCC | | | |
| | | CGACGCTTTCTTCGAAGACCGGATCG | | ATAACGGATCACAAGAAAGAG | | | |
| | | TAGCGGTGGATCCCGGCAGTCGAGT | | TTGTGGGCGCCATACCTAGAC | | | |
| | | ACCGGTGGCGGCGTGCGACTCTCGC | | ACGTGTGTAGTTGACATGAAG | | | |
| | | ACCGGATTCAAACGCATCACCAAAC | | AAGTTTGGATGTTGCATTCAA | | | |
| | | GATGGGTGCGTTCGCACACTTTGGAG | | ACGGACGGTGTAGCCGTGTCG | | | |
| | | TGGAAGCGCGATCGATATAGATCGC | | ATATCGATGAATAGACCGAAG | | | |
| | | GCAAATTGAGACGCGTCGAACTGAA | | AGGACGTCCGTCCGTCGAAAG | | | |
| | | CGAAGCCGCTGATAGACAACGCGTC | | GCAGCTCTACCCAAATCGTAT | | | |
| | | GAGTTAAATAGAGGAGTGCAAATAA | | ACAATCGACGCTTTCTTCGAA | | | |
| | | CGTGTCGCAACGGCGAGCAAGTTAA | | GACCGGATCGTAGCGGTGGAT | | | |
| | | GCTGTACACCGATTTCCGACTAAAGT | | CCCGGCAGTCGAGTACCGGTG | | | |
| | | GGTTCGATGCGCGCCAACTTCCGTTC | | GCGGCGTGCGACTCTCGCACC | | | |
| | | GAACGAACGCGCAAACTGACTAGAT | | GGATTCAAACGCATCACCAAA | | | |
| | | TGTCGTTCAACAAGTACATGATGACG | | CGATGGGTGCGTTCGCACACT | | | |
| | | GCTCGCATGAACGAGCGTATCGTTAG | | TTGGAGTGGAAGCGCGATCGA | | | |
| | | AGAAACGTTTCTCAGTGATACACCGT | | TATAGATCGCGCAAATTGAGA | | | |
| | | CGGATCGAGTGTTGGTGCTGTACGGA | | CGCGTCGAACTGAACGAAGCC | | | |
| | | GTGGGCGTCAACTTTGTCAACGTGGC | | GCTGATAGACAACGCGTCGAG | | | |
| | | CTGCTACAGCGGTCGCAAGTTCAAAC | | TTAAATAGAGGAGTGCAAATA | | | |
| | | ATACAGATTTGCTGCGTCGATTGAGA | | ACGTGTCGCAACGGCGAGCAA | | | |
| | | GCAAAACGCAACATCAGAGTGCAAC | | GTTAAGCTGTACACCGATTTCC | | | |
| | | TAGTAGACGAATCGTACACCAGCAA | | GACTAAAGTGGTTCGATGCGC | | | |
| | | AGCGTGCTCCGAGTGTAACGAGCTG | | GCCAACTTCCGTTCGAACGAA | | | |
| | | AGAAACGGAACGTACACTCGACTGC | | CGCGCAAACTGACTAGATTGT | | | |
| | | GGATGAACCATCGACTTCGTCGCGGC | | CGTTCAACAAGTACATGATGA | | | |
| | | GTCTGTCCAGACTGCAACGCCGACGT | | CGGCTCGCATGAACGAGCGTA | | | |
| | | CGAGAGGGACTACAACGCCGCCAAA | | TCGTTAGAGAAACGTTTCTCA | | | |
| | | AATATACTAGTTAATTATCAAAGGTC | | GTGATACACCGTCGGATCGAG | | | |
| | | CGCCGTCTGCAGGCCGAGCCCGAGC | | TGTTGGTGCTGTACGGAGTGG | | | |
| | | TGTGACTCGGGCGTAGCCACCGACA | | GCGTCAACTTTGTCAACGTGG | | | |
| | | CGGACCCGCACTCCATACGGTCCATT | | CCTGCTACAGCGGTCGCAAGT | | | |
| | | TTCCAATGCGCCGTATCGGAGCCCGG | | TCAAACATACAGATTTGCTGC | | | |
| | | GTCTTAGGAGGTTACCGTGCACCCTT | | GTCGATTGAGAGCAAAACGCA | | | |
| | | CTCACTCGGACGTGGGGTGATTGCGA | | ACATCAGAGTGCAACTAGTAG | | | |
| | | GACGTGCGGCCAACGCACGAATCGT | | ACGAATCGTACACCAGCAAAG | | | |
| | | GCTACGACGTGTCGACGACAAGTGT | | CGTGCTCCGAGTGTAACGAGC | | | |
| | | GCCACCGTTGCCATCGTTATCACGAA | | TGAGAAACGGAACGTACACTC | | | |
| | | CATTGTCAGTACTGTGAACGCAGTAG | | GACTGCGGATGAACCATCGAC | | | |
| | | TGAGACGTCAGTCGACTCGTAAACAT | | TTCGTCGCGGCGTCTGTCCAGA | | | |
| | | GATACTGTTCGTTACGTGTTTGTTCG | | CTGCAACGCCGACGTCGAGAG | | | |
| | | TCACCACCGTTTACGGCGATTGCTCG | | GGACTACAACGCCGCCAAAAA | | | |
| | | ACTACGACGCTGTCGTTTGTCCGAAA | | TATACTAGTTAATTATCAAAG | | | |
| | | AACCGGAAACTGCTTCGATTACGAA | | GTCCGCCGTCTGCAGGCCGAG | | | |
| | | CAGGGATCGGTGAGCCATCCCACG | | CCCGAGCTGTGACTCGGGCGT | | | |
| | | | | AGCCACCGACACGGACCCGCA | | | |
| | | | | CTCCATACGGTCCATTTTCCAA | | | |
| | | | | TGCGCCGTATCGGAGCCCGGG | | | |
| | | | | TCTTAG | | | |
| 99 | YP_ | ATGCCACCGTCGTGTAAATCGTGCGG | 219 | ATGCCACCGTCGTGTAAATCG | 250 | GTCGCGGCGTCTGTCCAGACT | 281 |
| | 001111029.1 | TGGTGAGGGTCACTCACGTCGCAGCT | | TGCGGTGGTGAGGGTCACTCA | | GCAACGCCGACGTCGAGCGG | |
| | | CAAAATCGTGTTTAAACTATCTTCCA | | CGTCGCAGCTCAAAATCGTGT | | GACTACAACGCCGCGAAGAA | |
| | | TCGATTCGACAAAGGAGCAACTGCG | | TTAAACTATCTTCCATCGATTC | | TATATTAGTTAATTTTCAAAG | |
| | | ATGATGTATCTACGGAAGATTTCGAT | | GACAAAGGAGCAACTGCGATG | | GTCCGCCGTCTGCAGGCCGAG | |
| | | GTTGGCGTCGTCAAGCGGGGTCTCAA | | ATGTATCTACGGAAGATTTCG | | CCCGAGCTGCGACTCGGGCGT | |
| | | TGGAGTGCTATGTCCACTGCTGGACG | | ATGTTGGCGTCGTCAAGCGGG | | AACCACCGAACGTGACTCGC | |
| | | AAGATGTGCGCCGACGTCTGACCGCT | | GTCTCAATGGAGTGCTATGTCC | | ACTCGATGACGATACGGATCA | |
| | | GAGATCAGATCGGATGTGGTCGAGC | | ACTGCTGGACGAAGATGTGCG | | TTTTTCAATGCGCCGTATCCG | |
| | | TCTCTAGACTTTACGTTCTACTCGGC | | CCGACGTCTGACCGCTGAGAT | | AGCTCGGGTCTTAGGAGGTTA | |
| | | GTGTTCGTCAACCACTGTTGGAACGC | | CAGATCGGATGTGGTCGAGCT | | TGTATGTGTCATTAGGTTACA | |
| | | ATACCCTAACAACGAATCGAACGGC | | CTCTAGACTTTACGTTCTACTC | | AGTCATTAAGACAAAATGAG | |
| | | GTTCGTACGTTTCAAGACATGATGAA | | GGCGTGTTCGTCAACCACTGTT | | TAACGGGGAAAACAAAATCG | |
| | | CTACGTTTACGCGCTCAAAGGCAAG | | GGAACGCATACCCTAACAACG | | AATGGGATGTAAGGCGTGGC | |
| | | GGACCGCACGCGCATAAATTCGACG | | AATCGAACGGCGTTCGTACGT | | GTGGCCCTCAGTCACCTTCGA | |
| | | ATATGGTGAGATATCACGACGGGAT | | TTCAAGACATGATGAACTACG | | AAGCGTAATGAAAATTCGAT | |
| | | TACGTACTACAACGGACGCCTGCGC | | TTTACGCGCTCAAAGGCAAGG | | ACGTCAACCAGCGTTACAAA | |
| | | AATTATTCGGTTCAAGAAATGGCTAA | | GACCGCACGCGCATAAATTCG | | ACCCTAAAACTCTTGATATGA | |
| | | AACGTATTGGACCGTGTTGAAGACG | | ACGATATGGTGAGATATCACG | | TTAGTACGCTTGCGTTGAATA | |
| | | AACATCACGACGCACGCCTATTCGCG | | ACGGGATTACGTACTACAACG | | ACGCAAGCGTACTACATGGTG | |
| | | CCTCGCCCGTTACTTCGGAGTCAAAC | | GACGCCTGCGCAATTATTCGG | | AAATGCTTCGAACACTTCGAC | |
| | | GAAACGATCCGACCCTCTTCGCAGCC | | TTCAAGAAATGGCTAAAACGT | | GTTCACCACCATTGTTGCGAG | |
| | | TATTATCACAAGGAGTTTGGTGACGA | | ATTGGACCGTGTTGAAGACGA | | AAAAACTTTTGTCTTCTATAT | |
| | | CGATATATCGAGAGTTTGTCGATTCA | | ACATCACGACGCACGCCTATT | | CGACAGTGCCGTTACGAGTAA | |
| | | TAAACGTATCGAGCGACTGGCATTCG | | CGCGCCTCGCCCGTTACTTCGG | | CGC | |
| | | ACAATACCGATGTGGATCGAAATAC | | AGTCAAACGAAACGATCCGAC | | | |
| | | AACGAGAGATGTATGCGCGCGGCGA | | CCTCTTCGCAGCCTATTATCAC | | | |
| | | ACAGTCCTTCGTCATCTTCCCGCAGC | | AAGGAGTTTGGTGACGACGAT | | | |
| | | CGAGTCACGGTTTGAAGCACGTCAC | | ATATCGAGAGTTTGTCGATTCA | | | |
| | | ATACACCAGCAGAGGTTGGCACGAG | | TAAACGTATCGAGCGACTGGC | | | |
| | | TTATTGAGACGCGTCACACCCTCGAA | | ATTCGACAATACCGATGTGGA | | | |
| | | CATCACATCTAACTGGCCCTCCATAA | | TCGAAATACAACGAGAGATGT | | | |
| | | CGGATCACAAGAAAGAGTTGTGGGC | | ATGCGCGCGGCGAACAGTCCT | | | |
| | | GCCGTACCTCGACACCAGCGTAGTG | | TCGTCATCTTCCCGCAGCCGAG | | | |
| | | GACATGAAAAAGTTTGGCTGCTGCAT | | TCACGGTTTGAAGCACGTCAC | | | |
| | | TCAGACGGACGGAGTCGCCGTTTCGT | | ATACACCAGCAGAGGTTGGCA | | | |
| | | TGTCGATGAACCGACCGAAGAGAAA | | CGAGTTATTGAGACGCGTCAC | | | |
| | | GCCTGCATTACGAAAGAAGCCTATAT | | ACCCTCGAACATCACATCTAA | | | |
| | | CGACATTACCTAAATCGTATGTAACG | | CTGGCCCTCCATAACGGATCA | | | |
| | | GATACATTCAACGAAGACCGCATCG | | CAAGAAAGAGTTGTGGGCGCC | | | |
| | | TGGCGGTGGATCCCGGTAGTCGTGTA | | GTACCTCGACACCAGCGTAGT | | | |
| | | CCGGTGGCGGCGTGCGACTCTCACAC | | GGACATGAAAAAGTTTGGCTG | | | |
| | | CGGATTCAAACGCATCACCAAACGA | | CTGCATTCAGACGGACGGAGT | | | |
| | | TGGGTGCGCTCACACACGTTGGAGTG | | CGCCGTTTCGTTGTCGATGAAC | | | |
| | | GAAACGGGAGCGATACAGATCGCGC | | CGACCGAAGAGAAAGCCTGCA | | | |
| | | AAATTGAGACGTGTCGAACTGGACG | | TTACGAAAGAAGCCTATATCG | | | |
| | | AAGCCGAAGATAGACATCGCGTCGA | | ACATTACCTAAATCGTATGTA | | | |
| | | GTTAGATATGGGAGTGCAGATCACG | | ACGGATACATTCAACGAAGAC | | | |
| | | TGTCGTAACGGCCATCGAGTCAAGCT | | CGCATCGTGGCGGTGGATCCC | | | |
| | | GTACACCGATTTCCGGCTAAAGTGGT | | GGTAGTCGTGTACCGGTGGCG | | | |
| | | TCGATGCGCGCCAACGTCCGTTCGAA | | GCGTGCGACTCTCACACCGGA | | | |
| | | CGAACGCGCAAACTAACTCGATTGTC | | TTCAAACGCATCACCAAACGA | | | |
| | | GTTCGACAAGTACGTTATGACGGCTC | | TGGGTGCGCTCACACACGTTG | | | |
| | | GCACCAACGAACGCATCGTGAGAGA | | GAGTGGAAACGGGAGCGATAC | | | |
| | | GACGTTTCTCGGCGATACACCATCGG | | AGATCGCGCAAATTGAGACGT | | | |
| | | ATCGGGTGTTGGTGCTGTATGGAGCG | | GTCGAACTGGACGAAGCCGAA | | | |
| | | GGAGTCAACTTTGTCAACGTGGCCTG | | GATAGACATCGCGTCGAGTTA | | | |
| | | CTACAGCGGTCGCAAGTTCAAACAC | | GATATGGGAGTGCAGATCACG | | | |
| | | ACAGATTTGTTACGTCGATTGAGATC | | TGTCGTAACGGCCATCGAGTC | | | |
| | | GAGACGCAACATCAGAGTTCGACTC | | AAGCTGTACACCGATTTCCGG | | | |
| | | GTCGACGAATCGTACACCAGCAAAG | | CTAAAGTGGTTCGATGCGCGC | | | |
| | | CGTGCTCCGACTGCAACGAGCTTCGA | | CAACGTCCGTTCGAACGAACG | | | |
| | | AACGGAACGTACACCCGACTGCGGA | | CGCAAACTAACTCGATTGTCG | | | |
| | | TGAACCATCGACTTCGTCGCGGCGTC | | TTCGACAAGTACGTTATGACG | | | |
| | | TGTCCAGACTGCAACGCCGACGTCG | | GCTCGCACCAACGAACGCATC | | | |
| | | AGCGGGACTACAACGCCGCGAAGAA | | GTGAGAGAGACGTTTCTCGGC | | | |
| | | TATATTAGTTAATTTTCAAAGGTCCG | | GATACACCATCGGATCGGGTG | | | |
| | | CCGTCTGCAGGCCGAGCCCGAGCTG | | TTGGTGCTGTATGGAGCGGGA | | | |
| | | CGACTCGGGCGTAACCACCGAACGT | | GTCAACTTTGTCAACGTGGCCT | | | |
| | | GACTCGCACTCGATGACGATACGGA | | GCTACAGCGGTCGCAAGTTCA | | | |
| | | TCATTTTTCAATGCGCCGTATCCGAG | | AACACACAGATTTGTTACGTC | | | |
| | | CTCGGGTCTTAGGAGGTTATGTATGT | | GATTGAGATCGAGACGCAACA | | | |
| | | GTCATTAGGTTACAAGTCATTAAGAC | | TCAGAGTTCGACTCGTCGACG | | | |
| | | AAAATGAGTAACGGGGAAAACAAAA | | AATCGTACACCAGCAAAGCGT | | | |
| | | TCGAATGGGATGTAAGGCGTGGCGT | | GCTCCGACTGCAACGAGCTTC | | | |
| | | GGCCCTCAGTCACCTTCGAAAGCGTA | | GAAACGGAACGTACACCCGAC | | | |
| | | ATGAAAATTCGATACGTCAACCAGC | | TGCGGATGAACCATCGACTTC | | | |
| | | GTTACAAAACCCTAAAACTCTTGATA | | GTCGCGGCGTCTGTCCAGACT | | | |
| | | TGATTAGTACGCTTGCGTTGAATAAC | | GCAACGCCGACGTCGAGCGGG | | | |
| | | GCAAGCGTACTACATGGTGAAATGC | | ACTACAACGCCGCGAAGAATA | | | |
| | | TTCGAACACTTCGACGTTCACCACCA | | TATTAGTTAATTTTCAAAGGTC | | | |
| | | TTGTTGCGAGAAAAACTTTTGTCTTC | | CGCCGTCTGCAGGCCGAGCCC | | | |
| | | TATATCGACAGTGCCGTTACGAGTAA | | GAGCTGCGACTCGGGCGTAAC | | | |
| | | CGC | | CACCGAACGTGACTCGCACTC | | | |
| | | | | GATGACGATACGGATCATTTTT | | | |
| | | | | CAATGCGCCGTATCCGAGCTC | | | |
| | | | | GGGTCTTAG | | | |
| 101 | YP_ | ATGAAAAAGAAGAAAAAAGGTGAGT | 220 | ATGAAAAAGAAGAAAAAAGG | 251 | TAGTGTGTTCTAACTGCGTGA | 282 |
| | 008052532.1 | TCCAAGAATTTAGGAATAATGAAAA | | TGAGTTCCAAGAATTTAGGAA | | GTTGCGAAAACAAAAAAATC | |
| | | GTCGGCATACAAAACTTTCAAAATAC | | TAATGAAAAGTCGGCATACAA | | GTATTTAGAACCAGAGATGCT | |
| | | CAATAAAATCTATTTTATATAATTAC | | AACTTTCAAAATACCAATAAA | | AATTCCTCAATAAATATAATG | |
| | | GACACTACACAACCAGTTATCAATA | | ATCTATTTTATATAATTACGAC | | AAATTAACAAGTTGTTGGATA | |
| | | ATTTAGTTTTTGAAATTAATGATTTA | | ACTACACAACCAGTTATCAAT | | GATAGCCAAACTCGTCCAACT | |
| | | ATGATACACGCTTACCAATTTATTCG | | AATTTAGTTTTTGAAATTAATG | | GCATTTCATTCTCATATTTCGT | |
| | | GTTATATGTGTTGAGTTGCTATACTA | | ATTTAATGATACACGCTTACCA | | CTTTCACCTGTTCCACTATCA | |
| | | ATAAACAGACATTACCTATAATAGAT | | ATTTATTCGGTTATATGTGTTG | | AAGAAGAACAGGAAAAAGTA | |
| | | GATATATTTATTTTATACTGCATAAA | | AGTTGCTATACTAATAAACAG | | AGACCATCGTAGGTGAAATTC | |
| | | AACATTAGGCACTCGTGATAATAGA | | ACATTACCTATAATAGATGAT | | CTACTATTGATTTTACATTTTT | |
| | | GGAAAGAAATGTAAAGATACAGCAC | | ATATTTATTTTATACTGCATAA | | TCTTATTTTTTTGCCCTGTAAA | |
| | | TTTTAGACACATTAGAAAAGTTTTAT | | AAACATTAGGCACTCGTGATA | | ATGGGCGTTTGAAATGAGAA | |
| | | TTGGAAGAATATCAACCTTTACTTAA | | ATAGAGGAAAGAAATGTAAAG | | AAGGTGTAAAGCCTCTAATTC | |
| | | CCACGAAAAAACTAATTTGAAAAAC | | ATACAGCACTTTTAGACACATT | | AAACTGTGGTTGAGTCTGGAA | |
| | | ACTACTTTTTTATTACCATATTTAGCA | | AGAAAAGTTTTATTTGGAAGA | | CCGACTTTTTAGACAAAAACG | |
| | | ACGCAAATTCATACTTCCTTATCCAA | | ATATCAACCTTTACTTAACCAC | | TTCATTATTTTGTATATAACA | |
| | | CAATGCACAAGAGCATTTTATTCAAC | | GAAAAAACTAATTTGAAAAAC | | AGCAGCGTGATACACAGAAA | |
| | | ATTTTTTGAGATTTATTAACACAACA | | ACTACTTTTTTATTACCATATT | | ATTTTCAATATGCTGTCTGCG | |
| | | ACAACTGCTATAACTGAAGATAAAT | | TAGCAACGCAAATTCATACTT | | TGCCAGTGTTGCGAGCGTCAT | |
| | | CCATCTTATTCAAATTGAAACACCAA | | CCTTATCCAACAATGCACAAG | | CAAGTGGATAAACCAAGAGC | |
| | | CTAATGAGTTTAGATAATGAAACAA | | AGCATTTTATTCAACATTTTTT | | AATGGAGCCGTGGGTTGAAA | |
| | | ATGAAATGTTTAATGAATGGAAAAT | | GAGATTTATTAACACAACAAC | | CCAAGTTTAATATGACGCAAA | |
| | | AACTCATTTACATAATATTTTTCCAC | | AACTGCTATAACTGAAGATAA | | CAACTCCTTGTGAATGTAT | |
| | | AAAACATTAAAAAATCAATACACTA | | ATCCATCTTATTCAAATTGAAA | | | |
| | | TGATGTGAAAGTTAGACCATTTGAAT | | CACCAACTAATGAGTTTAGAT | | | |
| | | ATTTGAAAGGAATGTTGTATATGAAT | | AATGAAACAAATGAAATGTTT | | | |
| | | GAAGTATTAGAAAAGGAAGAACATA | | AATGAATGGAAAATAACTCAT | | | |
| | | AATTATTTCAACCCTTACCGCTACGC | | TTACATAATATTTTTCCACAAA | | | |
| | | ACAAATATTATTCCAAAACATATTAT | | ACATTAAAAAATCAATACACT | | | |
| | | ATTGGATACTGCAAGTTTGGTTAGTT | | ATGATGTGAAAGTTAGACCAT | | | |
| | | TGTTTTGTCCTGAAACAGATAAGGAT | | TTGAATATTTGAAAGGAATGT | | | |
| | | GGAAACAAAAATAAAAAGGGGAATT | | TGTATATGAATGAAGTATTAG | | | |
| | | TATTAAGTAATATAAAAGATAATCA | | AAAAGGAAGAACATAAATTAT | | | |
| | | ATATGATATTTGGAATAGTTTTTTGA | | TTCAACCCTTACCGCTACGCAC | | | |
| | | ACTTGAACCATAAAATATTTAGAAAC | | AAATATTATTCCAAAACATATT | | | |
| | | CAACATTATCAATTCCATCATCAAAT | | ATATTGGATACTGCAAGTTTG | | | |
| | | ACAAACGGATGGTATTTCGTGTTGCT | | GTTAGTTTGTTTTGTCCTGAAA | | | |
| | | TGTTGTTTATTAGAAAAGATTTGAAA | | CAGATAAGGATGGAAACAAAA | | | |
| | | GATAAGAAATGGGGTTCAAGAGTTC | | ATAAAAAGGGGAATTTATTAA | | | |
| | | CAAGCATTCCAGAACAAGATTTTTAC | | GTAATATAAAAGATAATCAAT | | | |
| | | AACATTGAAGATTTATCAAAAGAAC | | ATGATATTTGGAATAGTTTTTT | | | |
| | | AATTGGATACACTGAAAGATAGAAA | | GAACTTGAACCATAAAATATT | | | |
| | | TATAGTTGGTTGCGACCCAGGAAAA | | TAGAAACCAACATTATCAATT | | | |
| | | CGCAGTCTTGTTTATATGATGGATAA | | CCATCATCAAATACAAACGGA | | | |
| | | AAATGGTAATAAACTACAATATACT | | TGGTATTTCGTGTTGCTTGTTG | | | |
| | | GCACCACAAAGAAAACGAGAAAGCA | | TTTATTAGAAAAGATTTGAAA | | | |
| | | AAGCAAAAACAAACCAACGAATATT | | GATAAGAAATGGGGTTCAAGA | | | |
| | | ATTAGAAGAAAAGAAACGACATAAA | | GTTCCAAGCATTCCAGAACAA | | | |
| | | ATTATTGAAAAAGAAACCGAGTTAT | | GATTTTTACAACATTGAAGATT | | | |
| | | CATTACAAAATAGCAAATCAGTTGAT | | TATCAAAAGAACAATTGGATA | | | |
| | | TATATCAAGTTCAAAACATATTTAGT | | CACTGAAAGATAGAAATATAG | | | |
| | | TGAAAAGCATAAACTTAACAAGGAA | | TTGGTTGCGACCCAGGAAAAC | | | |
| | | ACAACCGAATTTTACAAGAAGGAAA | | GCAGTCTTGTTTATATGATGGA | | | |
| | | CTTGGAGAAAAATGAATTTTAGGCA | | TAAAAATGGTAATAAACTACA | | | |
| | | ATATAGTTATGGTAAGAAATCAATA | | ATATACTGCACCACAAAGAAA | | | |
| | | GATACATTTCTTAATAAAATCCAAGA | | ACGAGAAAGCAAAGCAAAAA | | | |
| | | AACCTTTGGATCCAATATATTAATTG | | CAAACCAACGAATATTATTAG | | | |
| | | GTTATGGAAATTGGAGTAGAAGCAC | | AAGAAAAGAAACGACATAAA | | | |
| | | ACAAATGAAACATTTTATGCCTACTA | | ATTATTGAAAAAGAAACCGAG | | | |
| | | TGAATAAAGGATTAAGAAAACAAAT | | TTATCATTACAAAATAGCAAA | | | |
| | | CCATAAAAAGTATGATACAATAACA | | TCAGTTGATTATATCAAGTTCA | | | |
| | | ATCAATGAATGTAATACAAGCAAAA | | AAACATATTTAGTTGAAAAGC | | | |
| | | AGTGTTGCGATTGTAATAAAGACCTT | | ATAAACTTAACAAGGAAACAA | | | |
| | | GAATATTACAAGGATAAAGAAAACA | | CCGAATTTTACAAGAAGGAAA | | | |
| | | AGAAGGTATTTCGTCTTTTAGTGTGT | | CTTGGAGAAAAATGAATTTTA | | | |
| | | TCTAACTGCGTGAGTTGCGAAAACA | | GGCAATATAGTTATGGTAAGA | | | |
| | | AAAAAATCGTATTTAGAACCAGAGA | | AATCAATAGATACATTTCTTAA | | | |
| | | TGCTAATTCCTCAATAAATATAATGA | | TAAAATCCAAGAAACCTTTGG | | | |
| | | AATTAACAAGTTGTTGGATAGATAGC | | ATCCAATATATTAATTGGTTAT | | | |
| | | CAAACTCGTCCAACTGCATTTCATTC | | GGAAATTGGAGTAGAAGCACA | | | |
| | | TCATATTTCGTCTTTCACCTGTTCCAC | | CAAATGAAACATTTTATGCCT | | | |
| | | TATCAAAGAAGAACAGGAAAAAGTA | | ACTATGAATAAAGGATTAAGA | | | |
| | | AGACCATCGTAGGTGAAATTCCTACT | | AAACAAATCCATAAAAAGTAT | | | |
| | | ATTGATTTTACATTTTTTCTTATTTTT | | GATACAATAACAATCAATGAA | | | |
| | | TTGCCCTGTAAAATGGGCGTTTGAAA | | TGTAATACAAGCAAAAAGTGT | | | |
| | | TGAGAAAAGGTGTAAAGCCTCTAAT | | TGCGATTGTAATAAAGACCTT | | | |
| | | TCAAACTGTGGTTGAGTCTGGAACCG | | GAATATTACAAGGATAAAGAA | | | |
| | | ACTTTTTAGACAAAAACGTTCATTAT | | AACAAGAAGGTATTTCGTCTTT | | | |
| | | TTTGTATATAACAAGCAGCGTGATAC | | TAGTGTGTTCTAACTGCGTGAG | | | |
| | | ACAGAAAATTTTCAATATGCTGTCTG | | TTGCGAAAACAAAAAAATCGT | | | |
| | | CGTGCCAGTGTTGCGAGCGTCATCAA | | ATTTAGAACCAGAGATGCTAA | | | |
| | | GTGGATAAACCAAGAGCAATGGAGC | | TTCCTCAATAAATATAATGAA | | | |
| | | CGTGGGTTGAAACCAAGTTTAATATG | | ATTAACAAGTTGTTGGATAGA | | | |
| | | ACGCAAACAACTCCTTGTGAATGTAT | | TAGCCAAACTCGTCCAACTGC | | | |
| | | | | ATTTCATTCTCATATTTCGTCT | | | |
| | | | | TTCACCTGTTCCACTATCAAAG | | | |
| | | | | AAGAACAGGAAAAAGTAAGA | | | |
| | | | | CCATCGTAG | | | |
| 104.1 | YP_ | ATGAAAGAGGCAGTTAAAAATGTCA | 221 | ATGAAAGAGGCAGTTAAAAAT | 252 | GTTATAAGTATGGCACTGAAG | 283 |
| | 003987301.1 | AACCAAAAGTTCCCGCTAAAAAGAG | | GTCAAACCAAAAGTTCCCGCT | | TGGTTAATGTCAATGAATATT | |
| | | GATTATTACTGGAAGTAAAACTAAG | | AAAAAGAGGATTATTACTGGA | | TGACCACAAAAACCTGCTCAA | |
| | | AAAAAGGTTTTTGTCAAGAAGAAAC | | AGTAAAACTAAGAAAAAGGTT | | ACTGCGGCAAGATTAAAGAT | |
| | | CACCTGATAAGAAACCTCTTAAGAA | | TTTGTCAAGAAGAAACCACCT | | CTTGGAGCTAGCAAGATTTAC | |
| | | ACCCGTCAAAAAAACTGTTAAAACA | | GATAAGAAACCTCTTAAGAAA | | GAATGTGAGTCTTGTGGAATG | |
| | | TATAAACTAAAATCTATTTATGTTTC | | CCCGTCAAAAAAACTGTTAAA | | TACGCAGATCGTGATGAGAAT | |
| | | TAATAAAGATCTTAAAATGTCCAAAT | | ACATATAAACTAAAATCTATTT | | GCTGCCAAGAATATCCTAAAA | |
| | | GGATTCCCACTCCTAAAAAAGAATTT | | ATGTTTCTAATAAAGATCTTAA | | GTAGGATTAAAGCCTTGGTAC | |
| | | ACTGAAATTGAAACTAACAGTTGGT | | AATGTCCAAATGGATTCCCAC | | AAACAAAAATAGTCTAATAA | |
| | | ATGAACACAGGAAATTTGAGAATCC | | TCCTAAAAAAGAATTTACTGA | | AATCAGGGGTACATTCCGCTA | |
| | | CAATGGATCTCCTATACAATCCTATA | | AATTGAAACTAACAGTTGGTA | | GTACTCCACCCTACGGGTTAA | |
| | | ACAAGATTGTGCCTGTAGTTCCTCCG | | TGAACACAGGAAATTTGAGAA | | GCAAATGAGAATATCGAAAC | |
| | | GAATCTATTAAACAACAGAATTTAGC | | TCCCAATGGATCTCCTATACAA | | GGTATGCACAGGATTCTTCGA | |
| | | AAACAAACGTAAGAAAACAAATAGA | | TCCTATAACAAGATTGTGCCTG | | GTGATAATCTTAGGATGACTC | |
| | | CCTATTGTTTTTATTTCTTCTGAGAAA | | TAGTTCCTCCGGAATCTATTAA | | ACTAAGGAGATGACTAAAGT | |
| | | ATCAGAATTTATCCCACCAAAGAGC | | ACAACAGAATTTAGCAAACAA | | GTATCATTCAATATTGTATTG | |
| | | AACAAAAAATTTTACAAACTTGGTTT | | ACGTAAGAAAACAAATAGACC | | AACGGTATTCTTCCATAGAGA | |
| | | CGTTTATTTGCATGTATGTATAATAG | | TATTGTTTTTATTTCTTCTGAG | | GTTGATTTTTGGAGTATCCAG | |
| | | TTCTATTGATTACATTAACAGTAAGA | | AAAATCAGAATTTATCCCACC | | AAATATCAACTTTTTATGAGC | |
| | | AAGTTGTTCTTGAATCTGGTAGAATT | | AAAGAGCAACAAAAAATTTTA | | GGCGTTTGTGTTATCGGTAGT | |
| | | AATGTCGCTGCAACCAGGAAAGTTT | | CAAACTTGGTTTCGTTTATTTG | | AAATATACCTATGAATAAATA | |
| | | GTAATAAAATTAGCGTTAGAAAAGC | | CATGTATGTATAATAGTTCTAT | | TTTGGTTGAACATCTTTCCATT | |
| | | TCTAAAAACTATTAGAGACAATTTAA | | TGATTACATTAACAGTAAGAA | | AACTATAAACATAAAAGAG | |
| | | TCAAATCCACTAATCCATCAATAATG | | AGTTGTTCTTGAATCTGGTAGA | | | |
| | | ACCCATATAATGGACGAAGCGATTG | | ATTAATGTCGCTGCAACCAGG | | | |
| | | GTTTAGCTTGTTCAAATTATAAGACA | | AAAGTTTGTAATAAAATTAGC | | | |
| | | TGTTTAACCAATTATATTGAAGGTCA | | GTTAGAAAAGCTCTAAAAACT | | | |
| | | AATTAAAAAATTTGATATTAAACCTT | | ATTAGAGACAATTTAATCAAA | | | |
| | | GGTCTATCTCTAAAAGACGAAAGATT | | TCCACTAATCCATCAATAATG | | | |
| | | ATTGTCATTGAACCTGGATACTTTAA | | ACCCATATAATGGACGAAGCG | | | |
| | | AGGAAATTCTTTTTGTCCCACAGTTT | | ATTGGTTTAGCTTGTTCAAATT | | | |
| | | TTCCCAAAATGAAATCTTCTAAACCA | | ATAAGACATGTTTAACCAATT | | | |
| | | CTTATTATGATTGATAAAACTGTGAC | | ATATTGAAGGTCAAATTAAAA | | | |
| | | TTTGCAATACGATTCTGATACTAGAA | | AATTTGATATTAAACCTTGGTC | | | |
| | | AATATATCTTATTTGTACCTAGAGTC | | TATCTCTAAAAGACGAAAGAT | | | |
| | | ACTCCTAAGTATTCTGTCAATAAGGA | | TATTGTCATTGAACCTGGATAC | | | |
| | | AAAAAATAGTTGTGGTATTGATCCTG | | TTTAAAGGAAATTCTTTTTGTC | | | |
| | | GTCTTAGAGATTTCCTTACGGTTTAT | | CCACAGTTTTTCCCAAAATGA | | | |
| | | TCTGAAAATGAAACTCAATCAATTTG | | AATCTTCTAAACCACTTATTAT | | | |
| | | TCCTATTGAAATCGTAGTTAACACCA | | GATTGATAAAACTGTGACTTT | | | |
| | | CTAAAAATGAATACAAGAAAATTGA | | GCAATACGATTCTGATACTAG | | | |
| | | TAAGATCAACGAAATCATCAAAACT | | AAAATATATCTTATTTGTACCT | | | |
| | | AAACCAAATCTTAATAGTAAACGTA | | AGAGTCACTCCTAAGTATTCTG | | | |
| | | AAAAGAAACTTAATAGAGGCTTACG | | TCAATAAGGAAAAAAATAGTT | | | |
| | | TAAATATCATAGAAGAGTTACTAATA | | GTGGTATTGATCCTGGTCTTAG | | | |
| | | AGATGAAAGACATGCACTATAAAGT | | AGATTTCCTTACGGTTTATTCT | | | |
| | | TTCTCATGAATTAGTAAATACTTTTG | | GAAAATGAAACTCAATCAATT | | | |
| | | ATAAAATTTGTATAGGTAAACTAAAT | | TGTCCTATTGAAATCGTAGTTA | | | |
| | | GTAAAGTCTATCTTATCGAAAGCCAA | | ACACCACTAAAAATGAATACA | | | |
| | | TACTGTTCTAAAAAGTGCCTTGAAAA | | AGAAAATTGATAAGATCAACG | | | |
| | | GAAAACTTGCAACTCTGAGTTTTTAT | | AAATCATCAAAACTAAACCAA | | | |
| | | AGGTTTACTCAGAGATTAACTCATAT | | ATCTTAATAGTAAACGTAAAA | | | |
| | | GGGTTATAAGTATGGCACTGAAGTG | | AGAAACTTAATAGAGGCTTAC | | | |
| | | GTTAATGTCAATGAATATTTGACCAC | | GTAAATATCATAGAAGAGTTA | | | |
| | | AAAAACCTGCTCAAACTGCGGCAAG | | CTAATAAGATGAAAGACATGC | | | |
| | | ATTAAAGATCTTGGAGCTAGCAAGA | | ACTATAAAGTTTCTCATGAATT | | | |
| | | TTTACGAATGTGAGTCTTGTGGAATG | | AGTAAATACTTTTGATAAAATT | | | |
| | | TACGCAGATCGTGATGAGAATGCTG | | TGTATAGGTAAACTAAATGTA | | | |
| | | CCAAGAATATCCTAAAAGTAGGATT | | AAGTCTATCTTATCGAAAGCC | | | |
| | | AAAGCCTTGGTACAAACAAAAATAG | | AATACTGTTCTAAAAAGTGCC | | | |
| | | TCTAATAAAATCAGGGGTACATTCCG | | TTGAAAAGAAAACTTGCAACT | | | |
| | | CTAGTACTCCACCCTACGGGTTAAGC | | CTGAGTTTTTATAGGTTTACTC | | | |
| | | AAATGAGAATATCGAAACGGTATGC | | AGAGATTAACTCATATGGGTT | | | |
| | | ACAGGATTCTTCGAGTGATAATCTTA | | ATAAGTATGGCACTGAAGTGG | | | |
| | | GGATGACTCACTAAGGAGATGACTA | | TTAATGTCAATGAATATTTGAC | | | |
| | | AAGTGTATCATTCAATATTGTATTGA | | CACAAAAACCTGCTCAAACTG | | | |
| | | ACGGTATTCTTCCATAGAGAGTTGAT | | CGGCAAGATTAAAGATCTTGG | | | |
| | | TTTTGGAGTATCCAGAAATATCAACT | | AGCTAGCAAGATTTACGAATG | | | |
| | | TTTTATGAGCGGCGTTTGTGTTATCG | | TGAGTCTTGTGGAATGTACGC | | | |
| | | GTAGTAAATATACCTATGAATAAATA | | AGATCGTGATGAGAATGCTGC | | | |
| | | TTTGGTTGAACATCTTTCCATTAACT | | CAAGAATATCCTAAAAGTAGG | | | |
| | | ATAAACATAAAAGAG | | ATTAAAGCCTTGGTACAAACA | | | |
| | | | | AAAATAG | | | |
| 106 | YP_ | ATGGTTTATAATCAGGATCTCTCTAT | 222 | ATGGTTTATAATCAGGATCTCT | 253 | CAAAATGTGGTCGTATTAACG | 284 |
| | 003986680.1 | TTCTAATAAGACTTGTGGTATTTCCG | | CTATTTCTAATAAGACTTGTGG | | AAATAGGTTCAAGTAAAGTCC | |
| | | ATTGGTTGCCTCCTGTTAGAAACAAA | | TATTTCCGATTGGTTGCCTCCT | | ATAAATGTGCGTGTGGAATGT | |
| | | AAGATTTCAGATATCAAGTCCAATAG | | GTTAGAAACAAAAAGATTTCA | | ATGCAGGTCGAGATGAAAAT | |
| | | TTGGTTTGACATTCGTCGATGTCTCA | | GATATCAAGTCCAATAGTTGG | | GCAGCTAAGAATATTCTGAAA | |
| | | ATCCAAATATCAAGACAAAATTTGTT | | TTTGACATTCGTCGATGTCTCA | | AAAGGAATTAAAGAACATTA | |
| | | CCTGTTAACAATAAACAGATTGTTTT | | ATCCAAATATCAAGACAAAAT | | TTTATCAGCTCAAAGTGATAA | |
| | | AAAAGAACCTAATTGTATATATCTAA | | TTGTTCCTGTTAACAATAAACA | | AAATGTTACAGAATATAACA | |
| | | AATATGATCTCATTACTCTGAATCCT | | GATTGTTTTAAAAGAACCTAA | | ACAAAAAGTCTAACAATACA | |
| | | AGTTCTGATCATATTCGAATTTTACG | | TTGTATATATCTAAAATATGAT | | AACAAAGAAACTTAGTAGCC | |
| | | TGAATGGTTTAGTCTGTATGCTAAAA | | CTCATTACTCTGAATCCTAGTT | | TTCATCTCATCGTATTAGGAT | |
| | | TATACAATTATGCCATAAATTTAATT | | CTGATCATATTCGAATTTTACG | | TACGCTCTCATCCTAACAGTA | |
| | | AGGAAAATGTTTTATGTCAACAACA | | TGAATGGTTTAGTCTGTATGCT | | TTTCGGATTAACCCCCCGATT | |
| | | AGTTAATTAAATCCAGACTTCAATGT | | AAAATATACAATTATGCCATA | | TGATAGAGCGAACACATAATT | |
| | | GAAATTTATGATGAAATTATCATTCG | | AATTTAATTAGGAAAATGTTTT | | AATAAACTTATTGGTTTATCA | |
| | | GAAATTATCAAATGATAAAACCAAA | | ATGTCAACAACAAGTTAATTA | | AAGTCAACTTTAATAAACCAA | |
| | | CTTATTGAATCATTAAAATTATCTGA | | AATCCAGACTTCAATGTGAAA | | TAGCAATTGATTTGATTGACT | |
| | | TAGACCTCCAGAATTTTTGTTGGACT | | TTTATGATGAAATTATCATTCG | | ATTACGATACAAAAAAAATT | |
| | | ATGCGATTTCACATGCTTGTACATAC | | GAAATTATCAAATGATAAAAC | | GAAAAAAAAACATTCTGATA | |
| | | TGTAATTCGCAAGTCAAAAAATATTT | | CAAACTTATTGAATCATTAAA | | TAAAAAATAAATAGTAGTAA | |
| | | AAAAGACTCTAAAAATCCATACAAC | | ATTATCTGATAGACCTCCAGA | | TAGATTACGGGGGGTTAATCC | |
| | | AAGAAAACTCTTAGATTTAGATTATG | | ATTTTTGTTGGACTATGCGATT | | GAAATACTGTACAGATAAGA | |
| | | GTCTTTACATAAGAGTCGTAGAGTCA | | TCACATGCTTGTACATACTGTA | | CTAATTACAAGAGAGATTAAT | |
| | | TGCATATTTTACCTCCGAAATTATGT | | ATTCGCAAGTCAAAAAATATT | | GAGACTTTTTTAAACAACAAT | |
| | | GATAAAAATGTTTGTAATGGGTTATT | | TAAAAGACTCTAAAAATCCAT | | CACAA | |
| | | TTTGAAGCTCAATCCCAGCAAAAAC | | ACAACAAGAAAACTCTTAGAT | | | |
| | | ATACATGATATTTATATGTTTTGCGT | | TTAGATTATGGTCTTTACATAA | | | |
| | | AATGAGTTGGGATAGATTAACTGGT | | GAGTCGTAGAGTCATGCATAT | | | |
| | | AAATTTTTCGTCTATAAACCTGTGTT | | TTTACCTCCGAAATTATGTGAT | | | |
| | | AAAACGACTAATAAATATTCCTAAA | | AAAAATGTTTGTAATGGGTTA | | | |
| | | AAACAGTTTACAAGATGTGGTATTGA | | TTTTTGAAGCTCAATCCCAGCA | | | |
| | | TCCAGGTGTCAGAACTTTTTTGACAC | | AAAACATACATGATATTTATA | | | |
| | | TTTATTCATCAGAAAGAACTTATGAT | | TGTTTTGCGTAATGAGTTGGGA | | | |
| | | GTATGCGATTCTACATTTTACAATAA | | TAGATTAACTGGTAAATTTTTC | | | |
| | | CAAGATTAAAAGAATGTGGAGAAAG | | GTCTATAAACCTGTGTTAAAA | | | |
| | | ATTGATCGTATTAATTTTCTTTTACAC | | CGACTAATAAATATTCCTAAA | | | |
| | | AATCGTAAAAAACGTAAGAAACGTA | | AAACAGTTTACAAGATGTGGT | | | |
| | | AAAATGATAGACGTTTAAGAAAATC | | ATTGATCCAGGTGTCAGAACT | | | |
| | | ATTGGCAAAATATTATCGTAAAATCA | | TTTTTGACACTTTATTCATCAG | | | |
| | | GACAAAGAATCAAAGATCTTCACAC | | AAAGAACTTATGATGTATGCG | | | |
| | | AAAAACTGCGAAATATTTGGTTTCTA | | ATTCTACATTTTACAATAACAA | | | |
| | | GGTACAAAGAGATTTATATTGGGATT | | GATTAAAAGAATGTGGAGAAA | | | |
| | | TATGATACCAGCAAGAATCTCTCAGA | | GATTGATCGTATTAATTTTCTT | | | |
| | | TCCTAGAATTTCATTTTATGAAAAAC | | TTACACAATCGTAAAAAACGT | | | |
| | | GCAGAATAGCCACACTCAATCATGA | | AAGAAACGTAAAAATGATAGA | | | |
| | | ACTTTTCTTGACAAAACTTCAACAAG | | CGTTTAAGAAAATCATTGGCA | | | |
| | | TAGCTGATAGATTTGGGTCTTCAGTA | | AAATATTATCGTAAAATCAGA | | | |
| | | TTTGCTATTAACGAACATATGACCAC | | CAAAGAATCAAAGATCTTCAC | | | |
| | | AAAAACATGTTCAAAATGTGGTCGT | | ACAAAAACTGCGAAATATTTG | | | |
| | | ATTAACGAAATAGGTTCAAGTAAAG | | GTTTCTAGGTACAAAGAGATT | | | |
| | | TCCATAAATGTGCGTGTGGAATGTAT | | TATATTGGGATTTATGATACCA | | | |
| | | GCAGGTCGAGATGAAAATGCAGCTA | | GCAAGAATCTCTCAGATCCTA | | | |
| | | AGAATATTCTGAAAAAAGGAATTAA | | GAATTTCATTTTATGAAAAAC | | | |
| | | AGAACATTATTTATCAGCTCAAAGTG | | GCAGAATAGCCACACTCAATC | | | |
| | | ATAAAAATGTTACAGAATATAACAA | | ATGAACTTTTCTTGACAAAACT | | | |
| | | CAAAAAGTCTAACAATACAAACAAA | | TCAACAAGTAGCTGATAGATT | | | |
| | | GAAACTTAGTAGCCTTCATCTCATCG | | TGGGTCTTCAGTATTTGCTATT | | | |
| | | TATTAGGATTACGCTCTCATCCTAAC | | AACGAACATATGACCACAAAA | | | |
| | | AGTATTTCGGATTAACCCCCCGATTT | | ACATGTTCAAAATGTGGTCGT | | | |
| | | GATAGAGCGAACACATAATTAATAA | | ATTAACGAAATAGGTTCAAGT | | | |
| | | ACTTATTGGTTTATCAAAGTCAACTT | | AAAGTCCATAAATGTGCGTGT | | | |
| | | TAATAAACCAATAGCAATTGATTTGA | | GGAATGTATGCAGGTCGAGAT | | | |
| | | TTGACTATTACGATACAAAAAAAATT | | GAAAATGCAGCTAAGAATATT | | | |
| | | GAAAAAAAAACATTCTGATATAAAA | | CTGAAAAAAGGAATTAAAGAA | | | |
| | | AATAAATAGTAGTAATAGATTACGG | | CATTATTTATCAGCTCAAAGTG | | | |
| | | GGGGTTAATCCGAAATACTGTACAG | | ATAAAAATGTTACAGAATATA | | | |
| | | ATAAGACTAATTACAAGAGAGATTA | | ACAACAAAAAGTCTAACAATA | | | |
| | | ATGAGACTTTTTTAAACAACAATCAC | | CAAACAAAGAAACTTAG | | | |
| | | AA | | | | | |
| 107 | YP_ | ATGTTTTGGAACAACCAATGTGCCAA | 223 | ATGTTTTGGAACAACCAATGT | 254 | AATGTGAGGAGTATGGTAGTC | 285 |
| | 004894452.1 | ATTATCCAAATCTTTATGGATTCCTT | | GCCAAATTATCCAAATCTTTAT | | AATACTATGAGGTAGATGAAT | |
| | | CAAACATCATCAATTTAAAATCAAAC | | GGATTCCTTCAAACATCATCA | | CGTTTACGAGTAAAACATGCT | |
| | | AAAAAAAAATATTTTAAATGTGATA | | ATTTAAAATCAAACAAAAAAA | | GTAAATGTGGAAATATACAC | |
| | | ATCCAAATATTTCTTTTAATTATTTTA | | AATATTTTAAATGTGATAATCC | | AAAACATTAGGATCATCAAA | |
| | | CTGATGGTATTAAAATCAATAAAAA | | AAATATTTCTTTTAATTATTTT | | AATATATGATTGCATAAAATG | |
| | | AATTAAATTTAACGATATTAAAGTAA | | ACTGATGGTATTAAAATCAAT | | TAAAAACAAAATAGACAGAG | |
| | | CTGATAATCATGACAAAAAAGAGAA | | AAAAAAATTAAATTTAACGAT | | ATGTGAATGCATCAATATGTA | |
| | | ACTCAGAAACAAACTTATTAAAACA | | ATTAAAGTAACTGATAATCAT | | TTCTGATAAAAAATAAAGATA | |
| | | GAAAATACTAGATATATCAAAGAAC | | GACAAAAAAGAGAAACTCAG | | TACTATTAAAATAAAAAAAA | |
| | | AAAATAATCAAAAATGCAAATTAAA | | AAACAAACTTATTAAAACAGA | | CAAGGAGAGCGGTCTGCTCCT | |
| | | TAAACAAGAGCTAAATGACAAACAT | | AAATACTAGATATATCAAAGA | | TAATGGGAAATGTGACAAAT | |
| | | AAAAAGTTTCTTGATAAAATTGAGAT | | ACAAAATAATCAAAAATGCAA | | GTATAACAAATGTCTATAATT | |
| | | TAAAGTTAATCAATTGGACGGATTTA | | ATTAAATAAACAAGAGCTAAA | | AACATAGATTCATACATTTAC | |
| | | TTAGATCGCGAAGAATACAAATACT | | TGACAAACATAAAAAGTTTCT | | ATATTTTTTAAGTGATCGGCA | |
| | | CCCTAATAAAAAACAACAAAATATT | | TGATAAAATTGAGATTAAAGT | | ATATCTAATGTATCAGTTAAT | |
| | | ATTCAGAAATGGATTTATGATACAAC | | TAATCAATTGGACGGATTTATT | | AATATATCCGTTGATGTATTT | |
| | | ATCTATTTATAATAAATTAGTTAGTA | | AGATCGCGAAGAATACAAATA | | AAAATAATATTTTGCTGATCA | |
| | | GTTTTAATCAAATATACGACAAGTGT | | CTCCCTAATAAAAAACAACAA | | CAATAATTTTGTATCTTTTTAT | |
| | | CAAAAAATTGTAGACAATATGAATG | | AATATTATTCAGAAATGGATTT | | CTCTTTTTATTGAATAAGGTG | |
| | | TTGAAAATAAATTTTACCATTTGGGA | | ATGATACAACATCTATTTATAA | | TATAATCATCATTAATGTATA | |
| | | AAAATTATCAAACAAAATACAGAAT | | TAAATTAGTTAGTAGTTTTAAT | | TTGCAGATATATTAATGTTTT | |
| | | TTCCCATTAATTTTCACAAATTGAGA | | CAAATATACGACAAGTGTCAA | | TATGTATTTCATCGATTACAT | |
| | | GCATTAAAATGTAGTGATTTTATTCA | | AAAATTGTAGACAATATGAAT | | CTATTTCATCACCATATGTTG | |
| | | GGATTATGACAGGATTCCCTATTGTG | | GTTGAAAATAAATTTTACCATT | | | |
| | | TTATTGCTGATATTATAAAAGAATTT | | TGGGAAAAATTATCAAACAAA | | | |
| | | GTATCAAACATTAAATCCTGTATTAC | | ATACAGAATTTCCCATTAATTT | | | |
| | | AAAAATTTCTAAAGGACTAATAACA | | TCACAAATTGAGAGCATTAAA | | | |
| | | GAATTTAAATTTAAACCAAGAAAAT | | ATGTAGTGATTTTATTCAGGAT | | | |
| | | ATAATAGAATTTTTTCATATATTCCTT | | TATGACAGGATTCCCTATTGTG | | | |
| | | TAGAATCACATTATACAACTGTCAGA | | TTATTGCTGATATTATAAAAGA | | | |
| | | GGATTTTATCCATCTATATTTGGGAA | | ATTTGTATCAAACATTAAATCC | | | |
| | | AATTAAAACCAATGAAAAAATATTTT | | TGTATTACAAAAATTTCTAAA | | | |
| | | CATGGTTAGATATTAAACATGATTAT | | GGACTAATAACAGAATTTAAA | | | |
| | | AAGCTAATTTATGATAAGTATTCAAA | | TTTAAACCAAGAAAATATAAT | | | |
| | | CAAGTATTATATTCATGTTCCTAAAT | | AGAATTTTTTCATATATTCCTT | | | |
| | | ATGTTTATCGAAATGATTTGACAATA | | TAGAATCACATTATACAACTG | | | |
| | | AAAAAGCCTATAGCAATAATGGATC | | TCAGAGGATTTTATCCATCTAT | | | |
| | | CAGGAGCACGAACTTTTCAAACTATT | | ATTTGGGAAAATTAAAACCAA | | | |
| | | TATGGTTTAGATCATGTTGTCACCAT | | TGAAAAAATATTTTCATGGTTA | | | |
| | | TGGAGATGATTTAGGATCCACTTTCA | | GATATTAAACATGATTATAAG | | | |
| | | AAAAAAGATTATTAAAAATAGATAA | | CTAATTTATGATAAGTATTCAA | | | |
| | | TTTAAAATCCAAATTATCCAAACCAG | | ACAAGTATTATATTCATGTTCC | | | |
| | | GAAAATATAAGCATAATAAAAAACT | | TAAATATGTTTATCGAAATGAT | | | |
| | | AGATAGAAAAACCAAAGTGAAAAAA | | TTGACAATAAAAAAGCCTATA | | | |
| | | TGGAAATATAAGAGAGCCATTGATA | | GCAATAATGGATCCAGGAGCA | | | |
| | | GACATCATAAAAAGATGGATCATAT | | CGAACTTTTCAAACTATTTATG | | | |
| | | ACAACAAGAATTACATTATAAAACA | | GTTTAGATCATGTTGTCACCAT | | | |
| | | GCTAATTACTTATGTAGCCATTATGA | | TGGAGATGATTTAGGATCCAC | | | |
| | | TCGCGTGGTAGTTACTAATTTTAGTA | | TTTCAAAAAAAGATTATTAAA | | | |
| | | GTAAAAAAGTCGGATCAAAAAAGAA | | AATAGATAATTTAAAATCCAA | | | |
| | | TGATTTAGATGCAATAACAAAAAGA | | ATTATCCAAACCAGGAAAATA | | | |
| | | ATCCTTGGAAAATTATCTCATTATAG | | TAAGCATAATAAAAAACTAGA | | | |
| | | ATTTAGACAACGCCTTCTAACGAAAT | | TAGAAAAACCAAAGTGAAAAA | | | |
| | | GTGAGGAGTATGGTAGTCAATACTAT | | ATGGAAATATAAGAGAGCCAT | | | |
| | | GAGGTAGATGAATCGTTTACGAGTA | | TGATAGACATCATAAAAAGAT | | | |
| | | AAACATGCTGTAAATGTGGAAATAT | | GGATCATATACAACAAGAATT | | | |
| | | ACACAAAACATTAGGATCATCAAAA | | ACATTATAAAACAGCTAATTA | | | |
| | | ATATATGATTGCATAAAATGTAAAA | | CTTATGTAGCCATTATGATCGC | | | |
| | | ACAAAATAGACAGAGATGTGAATGC | | GTGGTAGTTACTAATTTTAGTA | | | |
| | | ATCAATATGTATTCTGATAAAAAATA | | GTAAAAAAGTCGGATCAAAAA | | | |
| | | AAGATATACTATTAAAATAAAAAAA | | AGAATGATTTAGATGCAATAA | | | |
| | | ACAAGGAGAGCGGTCTGCTCCTTAAT | | CAAAAAGAATCCTTGGAAAAT | | | |
| | | GGGAAATGTGACAAATGTATAACAA | | TATCTCATTATAGATTTAGACA | | | |
| | | ATGTCTATAATTAACATAGATTCATA | | ACGCCTTCTAACGAAATGTGA | | | |
| | | CATTTACATATTTTTTAAGTGATCGG | | GGAGTATGGTAGTCAATACTA | | | |
| | | CAATATCTAATGTATCAGTTAATAAT | | TGAGGTAGATGAATCGTTTAC | | | |
| | | ATATCCGTTGATGTATTTAAAATAAT | | GAGTAAAACATGCTGTAAATG | | | |
| | | ATTTTGCTGATCACAATAATTTTGTA | | TGGAAATATACACAAAACATT | | | |
| | | TCTTTTTATCTCTTTTTATTGAATAAG | | AGGATCATCAAAAATATATGA | | | |
| | | GTGTATAATCATCATTAATGTATATT | | TTGCATAAAATGTAAAAACAA | | | |
| | | GCAGATATATTAATGTTTTTATGTAT | | AATAGACAGAGATGTGAATGC | | | |
| | | TTCATCGATTACATCTATTTCATCAC | | ATCAATATGTATTCTGATAAA | | | |
| | | CATATGTTG | | AAATAAAGATATACTATTAAA | | | |
| | | | | ATAA | | | |
| 108 | YP_ | ATGGTATACATTAAAAACCGTATACC | 224 | ATGGTATACATTAAAAACCGT | 255 | TGGAAGCCAAAAAAGTTCAT | 286 |
| | 004895071.1 | AATCCATTATCCAAACAAAAATTGTG | | ATACCAATCCATTATCCAAAC | | AAATGCAAATGTGGAATGAA | |
| | | GACTACCCAATTGGGTTCCTTCTATT | | AAAAATTGTGGACTACCCAAT | | AACAGATCGAGATGTTAATGC | |
| | | AACAATTATTATGATTGTCTTAATAC | | TGGGTTCCTTCTATTAACAATT | | AGCAAAAAATATTCTCAAAGT | |
| | | AAATAGTTGGTTTGATATGAATATTT | | ATTATGATTGTCTTAATACAAA | | AGGATATCAGTATAACAGTAC | |
| | | GTAAAAATCCCAATAAATCTTCTGTT | | TAGTTGGTTTGATATGAATATT | | TCTGCACCAAGTTAACGATAT | |
| | | AAACGTCATCTTATAGATGCTCCCAT | | TGTAAAAATCCCAATAAATCT | | AGATATTATTGAAACAGACA | |
| | | TGTTCCTAATGAATCTTATAAACAAC | | TCTGTTAAACGTCATCTTATAG | | ATCATATAGTTACTTTTAATA | |
| | | AAATGAAATCCATTAAACGTTGCAA | | ATGCTCCCATTGTTCCTAATGA | | AAGTGATTAAAGAATATGAA | |
| | | ATCTAACAAAGAACCTGTTTTCATTT | | ATCTTATAAACAACAAATGAA | | GTTTTCGAAGTATAAAAAATA | |
| | | ATACCAGAAAGATTAGAATTTATCCT | | ATCCATTAAACGTTGCAAATCT | | TCATAGGCAAATTCCGCTAGA | |
| | | ACGGATGATCAAAAATGCATTTTAG | | AACAAAGAACCTGTTTTCATTT | | GCCTTAAATGAAATTATTAGG | |
| | | AAGAATGGTTTTGGGCTGCTACTAGA | | ATACCAGAAAGATTAGAATTT | | ATATTGAATTAAATTCTATGG | |
| | | ATGTATAATATTACAGTTAAATATAT | | ATCCTACGGATGATCAAAAAT | | GATTCAATTTAAATATTCTTG | |
| | | TCGAGATTTTTTGTTTAAAGACCATA | | GCATTTTAGAAGAATGGTTTTG | | ACCAATAATTCTATTGGAATA | |
| | | AACTCACTTGTCCTTTTGCATCTAAA | | GGCTGCTACTAGAATGTATAA | | ATTTTAATGGATCACTTTAAA | |
| | | GATATATTAATTAATTTTCCTTTTAG | | TATTACAGTTAAATATATTCGA | | AAAAATTGATATTTTCTTTTA | |
| | | AACTGTTTTAAAGGAAGACAGGGAC | | GATTTTTTGTTTAAAGACCATA | | AATATCAATAAATATAAAGTT | |
| | | AAACTTATGAAGTCAATGAAAAATC | | AACTCACTTGTCCTTTTGCATC | | TTCTAAAGCGGATCATGTGAA | |
| | | CCATTGTGGGTCATCTTTTGGATGAA | | TAAAGATATATTAATTAATTTT | | TTTATACTCATTATAAAATAA | |
| | | ATCATAGCACAAGTTGTTTCTAACTA | | CCTTTTAGAACTGTTTTAAAGG | | AATGATAATATATTAGATTTT | |
| | | TAAAGCAGCCATTTCCAATATACTTG | | AAGACAGGGACAAACTTATGA | | TATCAATCTAATATATCATAC | |
| | | TTGGTAATATTAAAAAATTTAAAATT | | AGTCAATGAAAAATCCCATTG | | AAAAATTATGGATTGATTCGG | |
| | | TCGACTTGGAAATATATCAGAGAAA | | TGGGTCATCTTTTGGATGAAAT | | GATTTAATATATCAATTTTTT | |
| | | AATATATTCTTAAAATAGAAAAAAA | | CATAGCACAAGTTGTTTCTAAC | | | |
| | | TTTGTTTTCGAATGGAACATTTTGTTC | | TATAAAGCAGCCATTTCCAAT | | | |
| | | AAGAACGTTCAAAGAATTAAAATCA | | ATACTTGTTGGTAATATTAAAA | | | |
| | | TCAGAACCCTTAATCAATATCCAACA | | AATTTAAAATTTCGACTTGGA | | | |
| | | CACTTGTACTTTACAATATAACAGAG | | AATATATCAGAGAAAAATATA | | | |
| | | ATATTGGAAAATATATCTTACTAGTT | | TTCTTAAAATAGAAAAAAATT | | | |
| | | CCTACCCACAAAGAAACCAAATCAT | | TGTTTTCGAATGGAACATTTTG | | | |
| | | ACGTCACCAATGATTTAGATTGCGGA | | TTCAAGAACGTTCAAAGAATT | | | |
| | | ATTGACCTAGGAGTGAGGACATTTGC | | AAAATCATCAGAACCCTTAAT | | | |
| | | CACTGTTTATTCTAGAAACAATGTCG | | CAATATCCAACACACTTGTACT | | | |
| | | TTACTATTGGAAACAAATTCAAGAA | | TTACAATATAACAGAGATATT | | | |
| | | AATTGATTCATGTCATAAGAAAATAG | | GGAAAATATATCTTACTAGTTC | | | |
| | | ATAAAATAAATGAGTTACTTGATTCA | | CTACCCACAAAGAAACCAAAT | | | |
| | | GACAATAATAAGTGTAAATTAAGAA | | CATACGTCACCAATGATTTAG | | | |
| | | AAGCCAAAAGAAAATATTATCGTCG | | ATTGCGGAATTGACCTAGGAG | | | |
| | | AATAAAAGATATTATTACCGATATGC | | TGAGGACATTTGCCACTGTTTA | | | |
| | | ACTATAAATCGGCTTACTATTTGGTT | | TTCTAGAAACAATGTCGTTACT | | | |
| | | ACTAATTACGATAATATACACATTGG | | ATTGGAAACAAATTCAAGAAA | | | |
| | | TAAATTAAGTACCAAAGGAATTCTTT | | ATTGATTCATGTCATAAGAAA | | | |
| | | CTAAGAATAACAAGAAAATATCCCG | | ATAGATAAAATAAATGAGTTA | | | |
| | | ACATACAAAACGTATGGTTGGTGTAT | | CTTGATTCAGACAATAATAAG | | | |
| | | TAAGTCCATATTTATTTAGGCAAAGA | | TGTAAATTAAGAAAAGCCAAA | | | |
| | | TTAAACCATATGGCACATAAATATGG | | AGAAAATATTATCGTCGAATA | | | |
| | | AGCTACCGTTTTTGAAGTGAATGAAT | | AAAGATATTATTACCGATATG | | | |
| | | ATCTAACCACAAAAACATGTTCTAAT | | CACTATAAATCGGCTTACTATT | | | |
| | | TGTGGAAAAATAAATGAACTGGAAG | | TGGTTACTAATTACGATAATAT | | | |
| | | CCAAAAAAGTTCATAAATGCAAATG | | ACACATTGGTAAATTAAGTAC | | | |
| | | TGGAATGAAAACAGATCGAGATGTT | | CAAAGGAATTCTTTCTAAGAA | | | |
| | | AATGCAGCAAAAAATATTCTCAAAG | | TAACAAGAAAATATCCCGACA | | | |
| | | TAGGATATCAGTATAACAGTACTCTG | | TACAAAACGTATGGTTGGTGT | | | |
| | | CACCAAGTTAACGATATAGATATTAT | | ATTAAGTCCATATTTATTTAGG | | | |
| | | TGAAACAGACAATCATATAGTTACTT | | CAAAGATTAAACCATATGGCA | | | |
| | | TTAATAAAGTGATTAAAGAATATGA | | CATAAATATGGAGCTACCGTT | | | |
| | | AGTTTTCGAAGTATAAAAAATATCAT | | TTTGAAGTGAATGAATATCTA | | | |
| | | AGGCAAATTCCGCTAGAGCCTTAAAT | | ACCACAAAAACATGTTCTAAT | | | |
| | | GAAATTATTAGGATATTGAATTAAAT | | TGTGGAAAAATAAATGAACTG | | | |
| | | TCTATGGGATTCAATTTAAATATTCT | | GAAGCCAAAAAAGTTCATAAA | | | |
| | | TGACCAATAATTCTATTGGAATAATT | | TGCAAATGTGGAATGAAAACA | | | |
| | | TTAATGGATCACTTTAAAAAAAATTG | | GATCGAGATGTTAATGCAGCA | | | |
| | | ATATTTTCTTTTAAATATCAATAAAT | | AAAAATATTCTCAAAGTAGGA | | | |
| | | ATAAAGTTTTCTAAAGCGGATCATGT | | TATCAGTATAACAGTACTCTGC | | | |
| | | GAATTTATACTCATTATAAAATAAAA | | ACCAAGTTAACGATATAGATA | | | |
| | | TGATAATATATTAGATTTTTATCAAT | | TTATTGAAACAGACAATCATA | | | |
| | | CTAATATATCATACAAAAATTATGGA | | TAGTTACTTTTAATAAAGTGAT | | | |
| | | TTGATTCGGGATTTAATATATCAATT | | TAAAGAATATGAAGTTTTCGA | | | |
| | | TTTT | | AGTATAA | | | |
| 110 | YP_ | ATGCGTTGTCATGAAATTTCATGGCC | 225 | ATGCGTTGTCATGAAATTTCAT | 256 | GTGATACGATTCTCGAACATC | 287 |
| | 001497907.1 | ATCCGGGAAACTCGATCAACATCTG | | GGCCATCCGGGAAACTCGATC | | CCGGAGAGCAATGGACTTCG | |
| | | AGAAACAAATTTTCGATCGGGAAAC | | AACATCTGAGAAACAAATTTT | | AAAACATGTGGAAGATGTGG | |
| | | ACAATGATATCGAACAATACATTTCG | | CGATCGGGAAACACAATGATA | | AAAGTCGAATCTTCCAGGGA | |
| | | ATTCATGGAAATAACATCGAACCCG | | TCGAACAATACATTTCGATTCA | | GAGAACGTCGAGGAACTTTC | |
| | | AACAGATCATCGAGACAACGAAGAT | | TGGAAATAACATCGAACCCGA | | AGAGAGTATAAATGTCTTTAT | |
| | | GAACACGGAAACAAGGAAAAAGGA | | ACAGATCATCGAGACAACGAA | | TGCGGATTGCATGCGCATCGA | |
| | | ACCTTCGAGACGTTATATCAATCCGT | | GATGAACACGGAAACAAGGA | | GATGTCCATGCGGCTCGCAAT | |
| | | TTTTCCTCGGAAAACTCTGGTTGTCT | | AAAAGGAACCTTCGAGACGTT | | ATTTTCATGAAATGGTATATC | |
| | | GAGTGTCCCAAGGATTTTAGAGCGA | | ATATCAATCCGTTTTTCCTCGG | | GAACTGAAAAAATAATATAA | |
| | | GAGCAACCTTCAAAGCTGCAGATGC | | AAAACTCTGGTTGTCTGAGTGT | | ATCATCGGGTTAGTCAAGTGC | |
| | | CTATAAAACTTCTCTGACGTTGTGGA | | CCCAAGGATTTTAGAGCGAGA | | TCCGAGAGGAGACAATCCCTT | |
| | | GAAAAGGACTCATTCGATCATTCGA | | GCAACCTTCAAAGCTGCAGAT | | ACATTGTGGAAAATGAAAGT | |
| | | GATGAAGTTCTCTACGAAGAGACGA | | GCCTATAAAACTTCTCTGACGT | | ATTTCGATACTTTTAATTACC | |
| | | GAACTAGACCGTGGTTATTGTTTCGG | | TGTGGAGAAAAGGACTCATTC | | ATTTGATGTATAAAATGAACG | |
| | | AATCGAGAAGAAAGTGAAGTTCGAA | | GATCATTCGAGATGAAGTTCT | | GCTGCAGCACGATTTTTGAAA | |
| | | GATACAGGTCTCAAAAAACGTGATG | | CTACGAAGAGACGAGAACTAG | | GTAAAAAATGGTTCACATATT | |
| | | GTAAGTTAATAATAACCGGAATAGA | | ACCGTGGTTATTGTTTCGGAAT | | TCAAAATGTATAAATAATCCC | |
| | | TGGAGACATCAGATTTTTCGAAAAAC | | CGAGAAGAAAGTGAAGTTCGA | | ACAAAATATAAATCGGCATAT | |
| | | CTCCGATCAATAAAGAACCCGTAGC | | AGATACAGGTCTCAAAAAACG | | AAATACTTCTGGCGCCGGGGA | |
| | | AGGATGCGAACTATCGAAAGATAGT | | TGATGGTAAGTTAATAATAAC | | TCCAGCTGATATTCTGACAGC | |
| | | TGTGGAGATTATTATCTTCATGTTCC | | CGGAATAGATGGAGACATCAG | | TGTGTTACCGAGTAAAACAGG | |
| | | TATCTTTCGAAAGAAGAAAGAACGG | | ATTTTTCGAAAAACCTCCGATC | | GCCAAAGCTCGTTGTTGAAAA | |
| | | AAAGGAGACAACTTCGTAGCTATTG | | AATAAAGAACCCGTAGCAGGA | | AGGTGATTATCGCAGGTTCAA | |
| | | ACCCCGGAGGCGTTGTTCCGTGGGCC | | TGCGAACTATCGAAAGATAGT | | C | |
| | | TTCTATGCTTCGAAGGGAGAATCGGG | | TGTGGAGATTATTATCTTCATG | | | |
| | | ATGTTTGGGAACTGAGATGAATGAG | | TTCCTATCTTTCGAAAGAAGA | | | |
| | | AGAATGTCGAATGTGTCGAAGAAGA | | AAGAACGGAAAGGAGACAAC | | | |
| | | TATCAACGATTGATAAGAAACTTTCT | | TTCGTAGCTATTGACCCCGGA | | | |
| | | GGACAACCTAAAGGCGATAAAAATT | | GGCGTTGTTCCGTGGGCCTTCT | | | |
| | | CGAACGATGTTATCGAGCGCCGAAA | | ATGCTTCGAAGGGAGAATCGG | | | |
| | | ACTTTTTAGGAAGAAGAAACGCATTC | | GATGTTTGGGAACTGAGATGA | | | |
| | | GAGATCATGAACATTACAGAGTCAT | | ATGAGAGAATGTCGAATGTGT | | | |
| | | CAACTTTCTTACGGATCGTTATGACG | | CGAAGAAGATATCAACGATTG | | | |
| | | GAATCATTTTACCAAAACTAAAAAC | | ATAAGAAACTTTCTGGACAAC | | | |
| | | GAAGAACATTTCGAAGGCGCTGAAA | | CTAAAGGCGATAAAAATTCGA | | | |
| | | TCCAAGGCGAACCACGACATGTTCG | | ACGATGTTATCGAGCGCCGAA | | | |
| | | ATATCAGTCATTATACTTTTCTGAGA | | AACTTTTTAGGAAGAAGAAAC | | | |
| | | CGTGTCGGAGAGCGTTGTATCGAAA | | GCATTCGAGATCATGAACATT | | | |
| | | GTGATACGATTCTCGAACATCCCGGA | | ACAGAGTCATCAACTTTCTTAC | | | |
| | | GAGCAATGGACTTCGAAAACATGTG | | GGATCGTTATGACGGAATCAT | | | |
| | | GAAGATGTGGAAAGTCGAATCTTCC | | TTTACCAAAACTAAAAACGAA | | | |
| | | AGGGAGAGAACGTCGAGGAACTTTC | | GAACATTTCGAAGGCGCTGAA | | | |
| | | AGAGAGTATAAATGTCTTTATTGCGG | | ATCCAAGGCGAACCACGACAT | | | |
| | | ATTGCATGCGCATCGAGATGTCCATG | | GTTCGATATCAGTCATTATACT | | | |
| | | CGGCTCGCAATATTTTCATGAAATGG | | TTTCTGAGACGTGTCGGAGAG | | | |
| | | TATATCGAACTGAAAAAATAATATA | | CGTTGTATCGAAAGTGATACG | | | |
| | | AATCATCGGGTTAGTCAAGTGCTCCG | | ATTCTCGAACATCCCGGAGAG | | | |
| | | AGAGGAGACAATCCCTTACATTGTG | | CAATGGACTTCGAAAACATGT | | | |
| | | GAAAATGAAAGTATTTCGATACTTTT | | GGAAGATGTGGAAAGTCGAAT | | | |
| | | AATTACCATTTGATGTATAAAATGAA | | CTTCCAGGGAGAGAACGTCGA | | | |
| | | CGGCTGCAGCACGATTTTTGAAAGTA | | GGAACTTTCAGAGAGTATAAA | | | |
| | | AAAAATGGTTCACATATTTCAAAATG | | TGTCTTTATTGCGGATTGCATG | | | |
| | | TATAAATAATCCCACAAAATATAAAT | | CGCATCGAGATGTCCATGCGG | | | |
| | | CGGCATATAAATACTTCTGGCGCCGG | | CTCGCAATATTTTCATGAAATG | | | |
| | | GGATCCAGCTGATATTCTGACAGCTG | | GTATATCGAACTGAAAAAATA | | | |
| | | TGTTACCGAGTAAAACAGGGCCAAA | | A | | | |
| | | GCTCGTTGTTGAAAAAGGTGATTATC | | | | | |
| | | GCAGGTTCAAC | | | | | |
| 111 | YP_ | ATGACATTGAAACCATGGTGGTCGA | 226 | ATGACATTGAAACCATGGTGG | 257 | CAGAGGAATACACTTCGAAG | 288 |
| | 001497530.1 | ACAAATCACAACAACTTACGAAAAT | | TCGAACAAATCACAACAACTT | | ACATGTGGTGTTTGCGGCCTG | |
| | | TATATCTTGGAACCCGACGAACACA | | ACGAAAATTATATCTTGGAAC | | ATTAATTATCTGTTGGGCAGC | |
| | | AATATATTTCAAAATGATACTACCAT | | CCGACGAACACAAATATATTT | | AAGAAGACATTCGAGTGTTTC | |
| | | CAAAAAAGTATTACGAACCCGAAAA | | CAAAATGATACTACCATCAAA | | TGCGGCAATGTATGCGATCGC | |
| | | GTAAAACTGAACCCTACGAAACAAC | | AAAGTATTACGAACCCGAAAA | | GATGTTCACGCAGCCCGCAAC | |
| | | AGAAAATGATGCTGATGAAATTTGC | | GTAAAACTGAACCCTACGAAA | | ATCCTCCTGAAATGGCTGAGC | |
| | | GGACGCTGCCAGATACTCTTATAACA | | CAACAGAAAATGATGCTGATG | | TCCGAGGCAGGTGCCCGGGTC | |
| | | TCGCGATAGCTGCGGTCAATGATAA | | AAATTTGCGGACGCTGCCAGA | | GCAGAGACCTTCCTTGCCTCT | |
| | | AACACACAAAGCGAATAAAATAGAA | | TACTCTTATAACATCGCGATAG | | CGGTCGAGTTAGACCTCTTGG | |
| | | CTTCAAAATGCGTTTTCTTCTCTCAA | | CTGCGGTCAATGATAAAACAC | | TGACGATACTTATATCGACAC | |
| | | AAGAAGAGATGATGATTCATATAAT | | ACAAAGCGAATAAAATAGAAC | | TGAGTGCTTATATTATTCGGC | |
| | | CAATTCTTTCAAAAACGCAGATGGTT | | TTCAAAATGCGTTTTCTTCTCT | | GTATACGGATGATATCCATAG | |
| | | GTTGGCAACACCTCAACCAATAAGA | | CAAAAGAAGAGATGATGATTC | | ATATTATCTGCTAGAATAATA | |
| | | AAACAAGCAATCTTTGAAGCAGCAA | | ATATAATCAATTCTTTCAAAAA | | TTTGATTAACTTATATGTTAG | |
| | | AGAATTTCAAGGCAGCATTTACGAA | | CGCAGATGGTTGTTGGCAACA | | TCTTTAGTAAATTTCTTATGA | |
| | | ATTGAAAAACAAAAACATCGATCAC | | CCTCAACCAATAAGAAAACAA | | AAACAGAACCATTCATGATTC | |
| | | TTCAAAATGAAGTTCAAGACGAAGA | | GCAATCTTTGAAGCAGCAAAG | | ATCTCGTGGTTCTCGCGATGT | |
| | | AACATCAACGACAACATGGGTTCTCT | | AATTTCAAGGCAGCATTTACG | | TCGTCACGATCTATCGTTTCTT | |
| | | CTCGGTATTGGTTCACATCTAAAACA | | AAATTGAAAAACAAAAACATC | | CCCTGGCGGATTCGAAAATAA | |
| | | TGACAACGGTGTTCTCACAATTTTAC | | GATCACTTCAAAATGAAGTTC | | CTTTTCTGTTGCAAATCCGGA | |
| | | CAAGAAGTCTGGGACAAATGCGATA | | AAGACGAAGAAACATCAACGA | | CAAAAAGGCATCATGGATAG | |
| | | TTTCGGTTTCCTTCCGTTCGAAGGCA | | CAACATGGGTTCTCTCTCGGTA | | ATTGTTTATATTTCGGAGTAA | |
| | | AACCCGACGCTGAATGTAGAATTCA | | TTGGTTCACATCTAAAACATG | | CGACACACTCGACCGTCG | |
| | | GAGAGATCCATATGGTGATCATTGGT | | ACAACGGTGTTCTCACAATTTT | | | |
| | | TGTTGGTGCCGGTGTATAAGACCACG | | ACCAAGAAGTCTGGGACAAAT | | | |
| | | AAGACGCAAATATCAAAACCTATCG | | GCGATATTTCGGTTTCCTTCCG | | | |
| | | TCGCCATCGATCCAGGAGTTCGCACT | | TTCGAAGGCAAACCCGACGCT | | | |
| | | CCTTTTGCGTGTTTTCGATCCGACGG | | GAATGTAGAATTCAGAGAGAT | | | |
| | | CGATTCGAAGTGCATCGGTGAAGAT | | CCATATGGTGATCATTGGTTGT | | | |
| | | ATGAACGCCAAACTCATAGACATAA | | TGGTGCCGGTGTATAAGACCA | | | |
| | | AGACTCGAGTGACCATAGTCGATCG | | CGAAGACGCAAATATCAAAAC | | | |
| | | ACGGATTTCGAAGTGCCAAGATGGTT | | CTATCGTCGCCATCGATCCAG | | | |
| | | CCCTAAAACGCAAATTACGGGAGCA | | GAGTTCGCACTCCTTTTGCGTG | | | |
| | | TCGACGAGTATTATTTCGAAAGCATC | | TTTTCGATCCGACGGCGATTCG | | | |
| | | AACGCGTGAGAGATGCGTTCCATTG | | AAGTGCATCGGTGAAGATATG | | | |
| | | GAAAATCATCAACGATATCACCGAC | | AACGCCAAACTCATAGACATA | | | |
| | | GAATGCTCTGGCGTGCTGTTGCCACC | | AAGACTCGAGTGACCATAGTC | | | |
| | | ATTCGAAACTCAACGCGTCAGCAGG | | GATCGACGGATTTCGAAGTGC | | | |
| | | ATGTTGAAAGCCAAAACAAACTGCT | | CAAGATGGTTCCCTAAAACGC | | | |
| | | CGATGCTTGGTATCAGCCACTACACT | | AAATTACGGGAGCATCGACGA | | | |
| | | TTCAGGATGCGAATGAAGGGAAAAT | | GTATTATTTCGAAAGCATCAA | | | |
| | | GTGAGGAAAAGGGATTGCTATACGA | | CGCGTGAGAGATGCGTTCCAT | | | |
| | | TGAACCGACAGAGGAATACACTTCG | | TGGAAAATCATCAACGATATC | | | |
| | | AAGACATGTGGTGTTTGCGGCCTGAT | | ACCGACGAATGCTCTGGCGTG | | | |
| | | TAATTATCTGTTGGGCAGCAAGAAG | | CTGTTGCCACCATTCGAAACTC | | | |
| | | ACATTCGAGTGTTTCTGCGGCAATGT | | AACGCGTCAGCAGGATGTTGA | | | |
| | | ATGCGATCGCGATGTTCACGCAGCCC | | AAGCCAAAACAAACTGCTCGA | | | |
| | | GCAACATCCTCCTGAAATGGCTGAGC | | TGCTTGGTATCAGCCACTACAC | | | |
| | | TCCGAGGCAGGTGCCCGGGTCGCAG | | TTTCAGGATGCGAATGAAGGG | | | |
| | | AGACCTTCCTTGCCTCTCGGTCGAGT | | AAAATGTGAGGAAAAGGGATT | | | |
| | | TAGACCTCTTGGTGACGATACTTATA | | GCTATACGATGAACCGACAGA | | | |
| | | TCGACACTGAGTGCTTATATTATTCG | | GGAATACACTTCGAAGACATG | | | |
| | | GCGTATACGGATGATATCCATAGATA | | TGGTGTTTGCGGCCTGATTAAT | | | |
| | | TTATCTGCTAGAATAATATTTGATTA | | TATCTGTTGGGCAGCAAGAAG | | | |
| | | ACTTATATGTTAGTCTTTAGTAAATT | | ACATTCGAGTGTTTCTGCGGCA | | | |
| | | TCTTATGAAAACAGAACCATTCATGA | | ATGTATGCGATCGCGATGTTC | | | |
| | | TTCATCTCGTGGTTCTCGCGATGTTC | | ACGCAGCCCGCAACATCCTCC | | | |
| | | GTCACGATCTATCGTTTCTTCCCTGG | | TGAAATGGCTGAGCTCCGAGG | | | |
| | | CGGATTCGAAAATAACTTTTCTGTTG | | CAGGTGCCCGGGTCGCAGAGA | | | |
| | | CAAATCCGGACAAAAAGGCATCATG | | CCTTCCTTGCCTCTCGGTCGAG | | | |
| | | GATAGATTGTTTATATTTCGGAGTAA | | TTAG | | | |
| | | CGACACACTCGACCGTCG | | | | | |
| 112 | YP_ | ATGAATATAAAAAATTATCATTGGTT | 227 | ATGAATATAAAAAATTATCAT | 258 | AATTTAGAGAAATATTAGAAT | 289 |
| | 003969989.1 | TAAAAATAAAAAACGAAAAAATATT | | TGGTTTAAAAATAAAAAACGA | | ATCAATGTAATAAATATAATA | |
| | | AATACTGATGGTTGGTTTAATTTAAA | | AAAAATATTAATACTGATGGT | | CTCAATTAAATATAGTAGATG | |
| | | ATCTATTAATAATAAAGGATTTAATG | | TGGTTTAATTTAAAATCTATTA | | AATCATATACAACAAAAACTT | |
| | | ATTATGACAATATAGAAAAACCAAT | | ATAATAAAGGATTTAATGATT | | GTAGCAATTGTAAGGAGTTAA | |
| | | AAATAAATTTTTATGTTGTAAAACAA | | ATGACAATATAGAAAAACCAA | | ATAATATAGGTTCATCAAAAA | |
| | | TTGAAATTTATCCTAATTTAGAACAA | | TAAATAAATTTTTATGTTGTAA | | AATATCAATGTAAAAGTTGTA | |
| | | AAAAATATTATTTTAAAGTGGATGGA | | AACAATTGAAATTTATCCTAAT | | ATTTTAAATGTGATAGAGATA | |
| | | TTTATTTATTAATATGTATAATCACA | | TTAGAACAAAAAAATATTATT | | TAAATGCATCAATAAATATTT | |
| | | CTAATTACTTTATAAATAATAATATT | | TTAAAGTGGATGGATTTATTTA | | ATTTAAAATAACTAATAATTA | |
| | | TATGATTTTACAAATAGAAAAATTAA | | TTAATATGTATAATCACACTAA | | TAGGCATGCTACTTGTCTTGA | |
| | | AAGTAATGTTAAAGAAATTTTAAATT | | TTACTTTATAAATAATAATATT | | ATAAAAGTTAGGTAATTTTCT | |
| | | TTAGAAAACTTAGAGATAATTACATT | | TATGATTTTACAAATAGAAAA | | ACGAATTATTACTGTTTTTAT | |
| | | AAAAATAAAAAAAATGAAACATGTA | | ATTAAAAGTAATGTTAAAGAA | | AAATAAATATAAACTTTTAAG | |
| | | AAGATGAAATTAATAAACATATGTT | | ATTTTAAATTTTAGAAAACTTA | | TAGCGGGAATTTGATAGAAA | |
| | | AGATGAAGCGATAAATCATTGTGTTT | | GAGATAATTACATTAAAAATA | | CATGTATTAATACAAATAATA | |
| | | CAAAACATAAAAGCTGTATAACAAA | | AAAAAAATGAAACATGTAAAG | | GGGACCTAGAAAATAATATT | |
| | | TTATTTTAGTAAAAATATTAAATTTT | | ATGAAATTAATAAACATATGT | | GATATTATAGATTATAATAAA | |
| | | TTAGAATTAGAAATATGAAAAAAAA | | TAGATGAAGCGATAAATCATT | | GATTTTATTGTGTCTAATTAT | |
| | | TCGTAGAAAAAAAATATTAATTATTG | | GTGTTTCAAAACATAAAAGCT | | GCAACAGAATTATTTTTAGAA | |
| | | AAAAAGGTTTATTTTCAAAAAAGAA | | GTATAACAAATTATTTTAGTAA | | AAACTTGATAATTTAAAAGAA | |
| | | AAATGGGTTTTGTTTAACAAAATTAC | | AAATATTAAATTTTTTAGAATT | | TTTAGTTTAACACTAGATTCT | |
| | | AAGAAATTAAAAGTTCTGAATCTATT | | AGAAATATGAAAAAAAATCGT | | TTTAAGTTATTGGAAAGTGAT | |
| | | GGTGTAAATAAAACAAGTATTTTACA | | AGAAAAAAAATATTAATTATT | | CTTATTCATATTAAAAAT | |
| | | ATATGATAAACATTTAAACAAATTTA | | GAAAAAGGTTTATTTTCAAAA | | | |
| | | CATTATTTATTCCAATTGAGAAAAAT | | AAGAAAAATGGGTTTTGTTTA | | | |
| | | ACTACAAATATAGAAAATAGAACAA | | ACAAAATTACAAGAAATTAAA | | | |
| | | AATGTGGAATAGATCCAGGCATTAG | | AGTTCTGAATCTATTGGTGTAA | | | |
| | | AACATTTTTAACTTGTTATTCAAAAA | | ATAAAACAAGTATTTTACAAT | | | |
| | | AGGAAATATTAGAAATAGGTGAAAA | | ATGATAAACATTTAAACAAAT | | | |
| | | TTGTTATGAAAAATATAAAAATAATT | | TTACATTATTTATTCCAATTGA | | | |
| | | ATAAAAAAATAGATAAAACGAGAGA | | GAAAAATACTACAAATATAGA | | | |
| | | ACTTTTTGATAAAAAAGAAATTAATA | | AAATAGAACAAAATGTGGAAT | | | |
| | | AAAAACAATTTACAAAATCAATAAA | | AGATCCAGGCATTAGAACATT | | | |
| | | TCATAATTTTGATAAAATTAGAAATT | | TTTAACTTGTTATTCAAAAAAG | | | |
| | | TAACAAAAGATTTACATTTTAAAGTT | | GAAATATTAGAAATAGGTGAA | | | |
| | | TCTAGTTATCTATGTAAAAAATTTAA | | AATTGTTATGAAAAATATAAA | | | |
| | | TATAATTACAATAGGAAAAATCAGT | | AATAATTATAAAAAAATAGAT | | | |
| | | ACCAGTAGTATTGTATTAAATACAAA | | AAAACGAGAGAACTTTTTGAT | | | |
| | | TAATTTAAATAAAATATCAAAAAGA | | AAAAAAGAAATTAATAAAAAA | | | |
| | | TGTATGTTATCATTATCACATTTTAA | | CAATTTACAAAATCAATAAAT | | | |
| | | ATTTAGAGAAATATTAGAATATCAAT | | CATAATTTTGATAAAATTAGA | | | |
| | | GTAATAAATATAATACTCAATTAAAT | | AATTTAACAAAAGATTTACAT | | | |
| | | ATAGTAGATGAATCATATACAACAA | | TTTAAAGTTTCTAGTTATCTAT | | | |
| | | AAACTTGTAGCAATTGTAAGGAGTTA | | GTAAAAAATTTAATATAATTA | | | |
| | | AATAATATAGGTTCATCAAAAAAAT | | CAATAGGAAAAATCAGTACCA | | | |
| | | ATCAATGTAAAAGTTGTAATTTTAAA | | GTAGTATTGTATTAAATACAA | | | |
| | | TGTGATAGAGATATAAATGCATCAAT | | ATAATTTAAATAAAATATCAA | | | |
| | | AAATATTTATTTAAAATAACTAATAA | | AAAGATGTATGTTATCATTATC | | | |
| | | TTATAGGCATGCTACTTGTCTTGAAT | | ACATTTTAAATTTAGAGAAAT | | | |
| | | AAAAGTTAGGTAATTTTCTACGAATT | | ATTAGAATATCAATGTAATAA | | | |
| | | ATTACTGTTTTTATAAATAAATATAA | | ATATAATACTCAATTAAATAT | | | |
| | | ACTTTTAAGTAGCGGGAATTTGATAG | | AGTAGATGAATCATATACAAC | | | |
| | | AAACATGTATTAATACAAATAATAG | | AAAAACTTGTAGCAATTGTAA | | | |
| | | GGACCTAGAAAATAATATTGATATTA | | GGAGTTAAATAATATAGGTTC | | | |
| | | TAGATTATAATAAAGATTTTATTGTG | | ATCAAAAAAATATCAATGTAA | | | |
| | | TCTAATTATGCAACAGAATTATTTTT | | AAGTTGTAATTTTAAATGTGAT | | | |
| | | AGAAAAACTTGATAATTTAAAAGAA | | AGAGATATAAATGCATCAATA | | | |
| | | TTTAGTTTAACACTAGATTCTTTTAA | | AATATTTATTTAAAATAA | | | |
| | | GTTATTGGAAAGTGATCTTATTCATA | | TTAAAAAT | | | |
| 113 | YP_ | ATGAAGGACAAGGGGGTGGTTCTGA | 228 | ATGAAGGACAAGGGGGTGGTT | 259 | CCCAGAAATTCACGGATGTCA | 290 |
| | 002154624.1 | GATGCAGGAAGGTAAGGATACTTCC | | CTGAGATGCAGGAAGGTAAGG | | AGGTTCGCCTATGTAACGAGG | |
| | | GAGTGAGGAACAGCGAGAAATACTC | | ATACTTCCGAGTGAGGAACAG | | CATATACGACGAAGCAATGT | |
| | | AAGTCTTGTTTCGGTATCCATCGACA | | CGAGAAATACTCAAGTCTTGT | | GGGTCATGCGGGGTTATCAAT | |
| | | CATCTATAACGAGTGTGTCAAGGCCG | | TTCGGTATCCATCGACACATCT | | CGCTCCATGACCTTGAACGAC | |
| | | AGAAGGACGGGGACATCGATGGAGC | | ATAACGAGTGTGTCAAGGCCG | | CGTTCATTCAACTGTGTGTCG | |
| | | TGGGGTTAGGGAGTGCTACAAGTGG | | AGAAGGACGGGGACATCGATG | | TGTGGTCTCTCGTCTTCAAGG | |
| | | CGAAAGGCTTTCACTACAAGAGAGG | | GAGCTGGGGTTAGGGAGTGCT | | GATGGTCATGCCGCGAGGAA | |
| | | ACTACTATGGACAGGGGAAGAATTG | | ACAAGTGGCGAAAGGCTTTCA | | CGTCGGTCTTCGCTCGTTACG | |
| | | GAAAGACGATTGCCCCAGCCATACC | | CTACAAGAGAGGACTACTATG | | ATTCCTTGTCTAAATGATCTA | |
| | | AAACAACAAGCCGTCGAGGAATTTT | | GACAGGGGAAGAATTGGAAA | | GGCAAATGTAGATACGGGGG | |
| | | TCAAAAATAAAAAGACGGGACTTAA | | GACGATTGCCCCAGCCATACC | | CTCCTATTGCTCCCTTGACGG | |
| | | ACTAGTCTCGGAGGGAAAGATAAGG | | AAACAACAAGCCGTCGAGGAA | | ATACCTGGAAAGTCATAAAG | |
| | | AGGTTTGACATCGGGTTCAAGAGCC | | TTTTTCAAAAATAAAAAGACG | | AAATACATTGGTTAGTCTACC | |
| | | GGTTCAAGTGTCGCCAAGAAACCATT | | GGACTTAAACTAGTCTCGGAG | | CGGAGTGAATGTAGAGGGTTT | |
| | | CCGTTTGAAAAATTCTTGATCAAGAA | | GGAAAGATAAGGAGGTTTGAC | | TCTATGGGTTTCCACCCTTTA | |
| | | GAAAGATTCGACGAATCCACTGAGC | | ATCGGGTTCAAGAGCCGGTTC | | CCGTATTGACCATAGGAGCTC | |
| | | AAGTCCCTGGTTTCCACTACGTACAA | | AAGTGTCGCCAAGAAACCATT | | GCTAGCTTCGTCCAGGACGAA | |
| | | GGGGAAATCGATGGATCTAAGGATT | | CCGTTTGAAAAATTCTTGATCA | | TTGAGTCGTTCCCGGGTTAAA | |
| | | ATGGGTAAGGTGCCAAAGGTGTTCA | | AGAAGAAAGATTCGACGAATC | | CTCCGGAAAAGAGAGAGAGA | |
| | | AGGATCGAGACGACCAAGGGTTCAC | | CACTGAGCAAGTCCCTGGTTTC | | GAGAGAGAGAAATGGAAGGG | |
| | | AAGGACCGAGATGAAGATTACCAGA | | CACTACGTACAAGGGGAAATC | | GGGAGGACGAGAAAGAAGGA | |
| | | ACCAAGACGGGGAAATACTACGCGA | | GATGGATCTAAGGATTATGGG | | ATAATAATAAACATACGTGG | |
| | | TTGTTTCGTTCGAGGTTCCCCAGACT | | TAAGGTGCCAAAGGTGTTCAA | | AGCTTACGGTGTGATAAAACA | |
| | | TCTCGTTTCCCCGAGATCCACGGTGA | | GGATCGAGACGACCAAGGGTT | | TAC | |
| | | CATGATCTCGTTTGATCCGGGATGCA | | CACAAGGACCGAGATGAAGAT | | | |
| | | AGACCTTTCAAACGTACCATTCTCCG | | TACCAGAACCAAGACGGGGAA | | | |
| | | GACGGAACATGGGGAGAGATAGGAA | | ATACTACGCGATTGTTTCGTTC | | | |
| | | CCTTCGAAAAACAAGAATTGTTGCTG | | GAGGTTCCCCAGACTTCTCGTT | | | |
| | | AGGAAGGCGGATAGGTTGAAGTCTA | | TCCCCGAGATCCACGGTGACA | | | |
| | | GGCTGGAGCTCGAGGGAAAAGATAA | | TGATCTCGTTTGATCCGGGATG | | | |
| | | GGACTCGAGGTGGAGAAGACGTACG | | CAAGACCTTTCAAACGTACCA | | | |
| | | CGAAGGAGGATGCTAAATTTGTTCG | | TTCTCCGGACGGAACATGGGG | | | |
| | | AGAAAGTGAGAAACCGAACGGCAGA | | AGAGATAGGAACCTTCGAAAA | | | |
| | | TCTGCACAATAAGGTCTGTTCGTGGG | | ACAAGAATTGTTGCTGAGGAA | | | |
| | | TGGTCAACACCTACCGATTGGTCCTC | | GGCGGATAGGTTGAAGTCTAG | | | |
| | | GTCCCGGAGTTCAAGACGAGTCAGA | | GCTGGAGCTCGAGGGAAAAGA | | | |
| | | TGGTCTCCTCTAAGAAACTCTACTCT | | TAAGGACTCGAGGTGGAGAAG | | | |
| | | AAGACGTGTAGAAAGATGATGACGT | | ACGTACGCGAAGGAGGATGCT | | | |
| | | GGTCTCACTACCGATTCAGGAGCAA | | AAATTTGTTCGAGAAAGTGAG | | | |
| | | GTTGATCGCGATGGCCCAGAAATTCA | | AAACCGAACGGCAGATCTGCA | | | |
| | | CGGATGTCAAGGTTCGCCTATGTAAC | | CAATAAGGTCTGTTCGTGGGT | | | |
| | | GAGGCATATACGACGAAGCAATGTG | | GGTCAACACCTACCGATTGGT | | | |
| | | GGTCATGCGGGGTTATCAATCGCTCC | | CCTCGTCCCGGAGTTCAAGAC | | | |
| | | ATGACCTTGAACGACCGTTCATTCAA | | GAGTCAGATGGTCTCCTCTAA | | | |
| | | CTGTGTGTCGTGTGGTCTCTCGTCTTC | | GAAACTCTACTCTAAGACGTG | | | |
| | | AAGGGATGGTCATGCCGCGAGGAAC | | TAGAAAGATGATGACGTGGTC | | | |
| | | GTCGGTCTTCGCTCGTTACGATTCCT | | TCACTACCGATTCAGGAGCAA | | | |
| | | TGTCTAAATGATCTAGGCAAATGTAG | | GTTGATCGCGATGGCCCAGAA | | | |
| | | ATACGGGGGCTCCTATTGCTCCCTTG | | ATTCACGGATGTCAAGGTTCG | | | |
| | | ACGGATACCTGGAAAGTCATAAAGA | | CCTATGTAACGAGGCATATAC | | | |
| | | AATACATTGGTTAGTCTACCCGGAGT | | GACGAAGCAATGTGGGTCATG | | | |
| | | GAATGTAGAGGGTTTTCTATGGGTTT | | CGGGGTTATCAATCGCTCCAT | | | |
| | | CCACCCTTTACCGTATTGACCATAGG | | GACCTTGAACGACCGTTCATTC | | | |
| | | AGCTCGCTAGCTTCGTCCAGGACGAA | | AACTGTGTGTCGTGTGGTCTCT | | | |
| | | TTGAGTCGTTCCCGGGTTAAACTCCG | | CGTCTTCAAGGGATGGTCATG | | | |
| | | GAAAAGAGAGAGAGAGAGAGAGAG | | CCGCGAGGAACGTCGGTCTTC | | | |
| | | AAATGGAAGGGGGGAGGACGAGAA | | GCTCGTTACGATTCCTTGTCTA | | | |
| | | AGAAGGAATAATAATAAACATACGT | | A | | | |
| | | GGAGCTTACGGTGTGATAAAACATA | | | | | |
| | | C | | | | | |
| 117 | YP_ | ATGAATAATTCAAAAATGGATTTACA | 229 | ATGAATAATTCAAAAATGGAT | 260 | TTGTTAATAGTGTAAAAGGAT | 291 |
| | 008052455.1 | AATTGAAGCAATAACTACATTACTTT | | TTACAAATTGAAGCAATAACT | | TGACAATATTAACACCAAATA | |
| | | CATCATTAGCAATTAATGAAACTAAA | | ACATTACTTTCATCATTAGCAA | | TAAATAATTTACCATTTCAAG | |
| | | ATAATAAAAATACAAAAATGGTTTA | | TTAATGAAACTAAAATAATAA | | TTTGCTGGGATAAAAATAGAA | |
| | | GGGGGTGCATTTTGAGATTAAAACA | | AAATACAAAAATGGTTTAGGG | | AATTTAAATATGAAAACATAA | |
| | | ATTACCTTTAATTATGTATAAAATTA | | GGTGCATTTTGAGATTAAAAC | | ACAAAAAGATAAAACTATTT | |
| | | AAAAATATTTGAAATTACAAGCGTTT | | AATTACCTTTAATTATGTATAA | | ATTGATTGCTTACAAAAACCA | |
| | | CAGTTTTCAACCCAAAATGAAGATG | | AATTAAAAAATATTTGAAATT | | AAACCAAGTTGGAAAGAAAC | |
| | | GTAGAATAAATAGTTGTATTGATGAA | | ACAAGCGTTTCAGTTTTCAACC | | ATTTATGTCAAATATACGAAC | |
| | | GATGAAGTAATTAAATTACTTATTGA | | CAAAATGAAGATGGTAGAATA | | ATTAGATTTATAGATATTCAC | |
| | | AAAGTTTGGTGGAAAAATAAAAAAA | | AATAGTTGTATTGATGAAGAT | | TTGGAATATAAGAATTACATA | |
| | | CCTAAAATTAGAATGTGGTATGATAT | | GAAGTAATTAAATTACTTATTG | | TTTGTCTATGTCCTATTTTAAA | |
| | | TTTAGCATTTGATTATATGTATGGGT | | AAAAGTTTGGTGGAAAAATAA | | CCTTCCAGAAAACATAAAATT | |
| | | GGGTTCCAATCAATATAAAAACAAC | | AAAAACCTAAAATTAGAATGT | | ATCTTTGAATGTATTGCTATTT | |
| | | AACTACAATAACAAGCGATAATACT | | GGTATGATATTTTAGCATTTGA | | ATGTATGATACTATATTATTT | |
| | | GGTAATTTGGCAATGTGTGTATATGC | | TTATATGTATGGGTGGGTTCCA | | AAATTGCACTTTTTTTTTGGTT | |
| | | TTACACAAATGAAATATTAGATATTA | | ATCAATATAAAAACAACAACT | | TAAGCATTATTAATCCACCAC | |
| | | ATAGGGATAAATCTTATGAAAATGG | | ACAATAACAAGCGATAATACT | | CAAAATAATTAACTTTACCTA | |
| | | TAAGATGAGTGATATACTTTTCAATA | | GGTAATTTGGCAATGTGTGTAT | | AAAACGATACATTTGGTTTCC | |
| | | AATTAAAAAATAAAAAATATAACAC | | ATGCTTACACAAATGAAATAT | | GTGTTAAATTATAAATGTAAA | |
| | | TAATAACAAAAAAGATTACTATTTCA | | TAGATATTAATAGGGATAAAT | | TACAATCTTTACCGAGATTAG | |
| | | TAGTGTTAAATAAAACCGATGCGAG | | CTTATGAAAATGGTAAGATGA | | TGTTTATGGTAGTAATATTTC | |
| | | TGATATAATTGTTAATAGTGTAAAAG | | GTGATATACTTTTCAATAAATT | | TTGGTGCTCCCCATTCAAACC | |
| | | GATTGACAATATTAACACCAAATATA | | AAAAAATAAAAAATATAACAC | | AATTATTTTCATTAA | |
| | | AATAATTTACCATTTCAAGTTTGCTG | | TAATAACAAAAAAGATTACTA | | | |
| | | GGATAAAAATAGAAAATTTAAATAT | | TTTCATAGTGTTAAATAAAACC | | | |
| | | GAAAACATAAACAAAAAGATAAAAC | | GATGCGAGTGATATAATTGTT | | | |
| | | TATTTATTGATTGCTTACAAAAACCA | | AATAGTGTAAAAGGATTGACA | | | |
| | | AAACCAAGTTGGAAAGAAACATTTA | | ATATTAACACCAAATATAAAT | | | |
| | | TGTCAAATATACGAACATTAGATTTA | | AATTTACCATTTCAAGTTTGCT | | | |
| | | TAGATATTCACTTGGAATATAAGAAT | | GGGATAAAAATAGAAAATTTA | | | |
| | | TACATATTTGTCTATGTCCTATTTTAA | | AATATGAAAACATAAACAAAA | | | |
| | | ACCTTCCAGAAAACATAAAATTATCT | | AGATAAAACTATTTATTGATTG | | | |
| | | TTGAATGTATTGCTATTTATGTATGA | | CTTACAAAAACCAAAACCAAG | | | |
| | | TACTATATTATTTAAATTGCACTTTTT | | TTGGAAAGAAACATTTATGTC | | | |
| | | TTTTGGTTTAAGCATTATTAATCCAC | | AAATATACGAACATTAGATTT | | | |
| | | CACCAAAATAATTAACTTTACCTAAA | | ATAG | | | |
| | | AACGATACATTTGGTTTCCGTGTTAA | | | | | |
| | | ATTATAAATGTAAATACAATCTTTAC | | | | | |
| | | CGAGATTAGTGTTTATGGTAGTAATA | | | | | |
| | | TTTCTTGGTGCTCCCCATTCAAACCA | | | | | |
| | | ATTATTTTCATTAA | | | | | |
| 118 | YP_ | ATGGTTTTTTATAGTTGCGAAAAATG | 230 | ATGGTTTTTTATAGTTGCGAAA | 261 | CAAATGTATCGTTTTTAGGTA | 292 |
| | 008052454.1 | TGGTAAACAATTTAGTCAAAAGGGA | | AATGTGGTAAACAATTTAGTC | | AAGTTAATTATTTTGGTGGTG | |
| | | CATTACAATAAACATATCAATAAAA | | AAAAGGGACATTACAATAAAC | | GATTAATAATGCTTAAACCAA | |
| | | AACTTCCTTGTGTTAATGAAACAAAA | | ATATCAATAAAAAACTTCCTT | | AAAAAAAGTGCAATTTAAAT | |
| | | CTTAAAGAAATTATAGGTGTAGTTGT | | GTGTTAATGAAACAAAACTTA | | AATATAGTATCATACATAAAT | |
| | | TAATGAAAAATTGAATGAAATTAAT | | AAGAAATTATAGGTGTAGTTG | | AGCAATACATTCAAAGATAAT | |
| | | AATAATAAAGAAAAAATTGATTTATT | | TTAATGAAAAATTGAATGAAA | | TTTATGTTTTCTGGAAGGTTT | |
| | | TATTTCAAAAGATTTAGAAGAAACTA | | TTAATAATAATAAAGAAAAAA | | AAAATAGGACATAGACAAAT | |
| | | TAAACACAGAAATACCAACTATGAC | | TTGATTTATTTATTTCAAAAGA | | ATGTAATTCTTATATTCCAAG | |
| | | AACAAAACATAATTTAGGGCAATAT | | TTTAGAAGAAACTATAAACAC | | TGAATATCTATAAATCTAATG | |
| | | TTTACAACCCATAATGAACTCAAAGA | | AGAAATACCAACTATGACAAC | | TTCGTATATTTGACATAAATG | |
| | | AAAGGTATTTGAGTTTATCTTAAATA | | AAAACATAATTTAGGGCAATA | | TTTCTTTCCAACTTGGTTTTGG | |
| | | CTCCATCTAATATTTTAGAACCATCT | | TTTTACAACCCATAATGAACTC | | TTTTTGTAAGCAATCAATAAA | |
| | | ATGGGACAAGGTGATTTAATTACATT | | AAAGAAAAGGTATTTGAGTTT | | TAGTTTTATCTTTTTGTTTATG | |
| | | TATTACAGATAAAATACCAAGTATA | | ATCTTAAATACTCCATCTAATA | | TTTTCATATTTAAATTTTCTAT | |
| | | ACATTTGATATGTATGAAATTGATAA | | TTTTAGAACCATCTATGGGAC | | TTTTATCCCAGCAAACTTGAA | |
| | | AAAAATTAAATTATTGGATAAAATA | | AAGGTGATTTAATTACATTTAT | | ATGGTAAATTATTTATATTTG | |
| | | CAAAAAGATAAGATTAATTATGGAG | | TACAGATAAAATACCAAGTAT | | GTGTTAATATTGTCAATCCTT | |
| | | ATTTTATGACACAAACAATTACAAAA | | AACATTTGATATGTATGAAATT | | TTACACTATTAACAATTATAT | |
| | | ACATACAAAACAATAGTAGGAAATC | | GATAAAAAAATTAAATTATTG | | CACTCGCATCGGTTTTATTTA | |
| | | CACCTTATGTTAGAACTAAAAAAGG | | GATAAAATACAAAAAGATAAG | | ACACTATGAAATAGTAATCTT | |
| | | GAACTTATATATTGATTTTACCGAAA | | ATTAATTATGGAGATTTTATGA | | TTTTGTTATTAGTGTTATATTT | |
| | | AATGTTATAATTTACTTGAAGATAAT | | CACAAACAATTACAAAAACAT | | TTTATTTTTTAATTTATTGAAA | |
| | | GGCGAATTGATATTTATTGTGCCGTC | | ACAAAACAATAGTAGGAAATC | | AGTATATCACTCAT | |
| | | TGATTTTCTTAAATTAACAAGTGCAT | | CACCTTATGTTAGAACTAAAA | | | |
| | | CAAAATTATTAAATGTTATGATGACA | | AAGGGAACTTATATATTGATTT | | | |
| | | AATGGAACATTTACTCATATATTCCA | | TACCGAAAAATGTTATAATTT | | | |
| | | TAATCATAACGAAAAAATGTTTGATA | | ACTTGAAGATAATGGCGAATT | | | |
| | | ATGCATCTATTGATGTTATTGTTTTTA | | GATATTTATTGTGCCGTCTGAT | | | |
| | | GATATTGTAAAAATAGTTTAATTGAT | | TTTCTTAAATTAACAAGTGCAT | | | |
| | | AAAAAAGTATTATATAACGACAAAT | | CAAAATTATTAAATGTTATGAT | | | |
| | | TACTCTATATTACAAACAGCAACGGA | | GACAAATGGAACATTTACTCA | | | |
| | | TTAATTACTTTTGGAGAAGAAGAAA | | TATATTCCATAATCATAACGA | | | |
| | | ATAATAATAGTGTCTTGTTTCAAGAA | | AAAAATGTTTGATAATGCATC | | | |
| | | TATTTTGACATTTATGTTGGTCTTGTT | | TATTGATGTTATTGTTTTTAGA | | | |
| | | AGTGGAAAAGAAGAAGTTTATAAAA | | TATTGTAAAAATAGTTTAATTG | | | |
| | | ATGAGGAGCTTGGTAATATTGAAGTT | | ATAAAAAAGTATTATATAACG | | | |
| | | TTAAATGGTGAAGATAAAATTGATA | | ACAAATTACTCTATATTACAA | | | |
| | | AATATATTTATATTGAAAACTACCCT | | ACAGCAACGGATTAATTACTT | | | |
| | | TGTGATAATGAGAAAATTAATAAAC | | TTGGAGAAGAAGAAAATAATA | | | |
| | | ATTTATCACATCACAAAAAAGAACTT | | ATAGTGTCTTGTTTCAAGAATA | | | |
| | | ATTGAAAGAGGAATACGAAAGTTTA | | TTTTGACATTTATGTTGGTCTT | | | |
| | | ATGAAAATAATTGGTTTGAATGGGG | | GTTAGTGGAAAAGAAGAAGTT | | | |
| | | AGCACCAAGAAATATTACTACCATA | | TATAAAAATGAGGAGCTTGGT | | | |
| | | AACACTAATCTCGGTAAAGATTGTAT | | AATATTGAAGTTTTAAATGGT | | | |
| | | TTACATTTATAATTTAACACGGAAAC | | GAAGATAAAATTGATAAATAT | | | |
| | | CAAATGTATCGTTTTTAGGTAAAGTT | | ATTTATATTGAAAACTACCCTT | | | |
| | | AATTATTTTGGTGGTGGATTAATAAT | | GTGATAATGAGAAAATTAATA | | | |
| | | GCTTAAACCAAAAAAAAAGTGCAAT | | AACATTTATCACATCACAAAA | | | |
| | | TTAAATAATATAGTATCATACATAAA | | AAGAACTTATTGAAAGAGGAA | | | |
| | | TAGCAATACATTCAAAGATAATTTTA | | TACGAAAGTTTAATGAAAATA | | | |
| | | TGTTTTCTGGAAGGTTTAAAATAGGA | | ATTGGTTTGAATGGGGAGCAC | | | |
| | | CATAGACAAATATGTAATTCTTATAT | | CAAGAAATATTACTACCATAA | | | |
| | | TCCAAGTGAATATCTATAAATCTAAT | | ACACTAATCTCGGTAAAGATT | | | |
| | | GTTCGTATATTTGACATAAATGTTTC | | GTATTTACATTTATAATTTAAC | | | |
| | | TTTCCAACTTGGTTTTGGTTTTTGTAA | | ACGGAAACCAAATGTATCGTT | | | |
| | | GCAATCAATAAATAGTTTTATCTTTT | | TTTAGGTAAAGTTAATTATTTT | | | |
| | | TGTTTATGTTTTCATATTTAAATTTTC | | GGTGGTGGATTAATAATGCTT | | | |
| | | TATTTTTATCCCAGCAAACTTGAAAT | | AAACCAAAAAAAAAGTGCAAT | | | |
| | | GGTAAATTATTTATATTTGGTGTTAA | | TTAAATAATATAGTATCATAC | | | |
| | | TATTGTCAATCCTTTTACACTATTAA | | ATAAATAGCAATACATTCAAA | | | |
| | | CAATTATATCACTCGCATCGGTTTTA | | GATAATTTTATGTTTTCTGGAA | | | |
| | | TTTAACACTATGAAATAGTAATCTTT | | GGTTTAAAATAGGACATAGAC | | | |
| | | TTTGTTATTAGTGTTATATTTTTTATT | | AAATATGTAATTCTTATATTCC | | | |
| | | TTTTAATTTATTGAAAAGTATATCAC | | AAGTGAATATCTATAA | | | |
| | | TCAT | | | | | |
| 119 | WP_ | GTGAAACGATTACAGGCATTTAAATT | 231 | GTGAAACGATTACAGGCATTT | 262 | ATACAGCGAAAGAGAACCGC | 293 |
| | 105908056.1 | CCAGTTAAGACCAGGTGGTCAACAG | | AAATTCCAGTTAAGACCAGGT | | CTGTCACAAAGTAAATTCAGA | |
| | | GAGTGTGAAATGAGGCGCTTCGCAG | | GGTCAACAGGAGTGTGAAATG | | TGCCAGGTATGTGGATATACA | |
| | | GCGCATGTCGTTTCGTTTTCAATCGT | | AGGCGCTTCGCAGGCGCATGT | | GCGAACGCCGATGTAAATGG | |
| | | GCTCTGGCACGTCAGAATGAGAATC | | CGTTTCGTTTTCAATCGTGCTC | | CGCTCGCAACATTTTAGCGGC | |
| | | ATGAGGCCGGTAATAAATACATCCCT | | TGGCACGTCAGAATGAGAATC | | GGGGCACGCCGTTCTTGCCTG | |
| | | TACGGGAAAATGGCTTCCTGGCTGGT | | ATGAGGCCGGTAATAAATACA | | TGGAGAGATGGTGCAGTCAG | |
| | | TGAGTGGAAAAATGCCACTGAAACG | | TCCCTTACGGGAAAATGGCTT | | GCCGCCCGTTGAAGCAGGAA | |
| | | CAATGGCTTAAAGATTCTCCCTCACA | | CCTGGCTGGTTGAGTGGAAAA | | CCCACCGAAATGATTCAGGCG | |
| | | GCCATTGCAACAGTCACTGAAAGAC | | ATGCCACTGAAACGCAATGGC | | ACAGCCAGAACGTAGCAGGG | |
| | | CTTGAGCGGGCTTACAAAAACTTCTT | | TTAAAGATTCTCCCTCACAGCC | | ATCCACGTCCTTCAGGGCGTG | |
| | | CCGGAAGCGGGCTGCTTTTCCCCGAT | | ATTGCAACAGTCACTGAAAGA | | GAGGATGTCAATATCGGTTTC | |
| | | TCAAAAAGCGGGGACAGAATGATGC | | CCTTGAGCGGGCTTACAAAAA | | TGTCAGCGATTAAATTCAACA | |
| | | ATTCCGCTACCCGCAGGGCGTTAAGC | | CTTCTTCCGGAAGCGGGCTGCT | | CCACGTAACAAACGCCTCCGG | |
| | | TCGATCAGGAAAACAGCCGTATTTTT | | TTTCCCCGATTCAAAAAGCGG | | CAATTAATCCCCAAAACGCAG | |
| | | CTGCCGAAACTGGGCTGGATGCGCT | | GGACAGAATGATGCATTCCGC | | AACCAATCCCGACCAGCGTCA | |
| | | ACCGGAACAGCCGTCAGGTCACGGG | | TACCCGCAGGGCGTTAAGCTC | | ATCCACTTGCCGTTACCAGAA | |
| | | TGTTGTGAAAAATGTCACTGTCAGCC | | GATCAGGAAAACAGCCGTATT | | ATGCCACCACCGCCGCGTCTC | |
| | | AGTCCTGCGGTAAGTGGTACATCAGT | | TTTCTGCCGAAACTGGGCTGG | | GCTCACGCTCATTATGCAGCG | |
| | | ATTCAGACAGAAAGTGAAGTATCAA | | ATGCGCTACCGGAACAGCCGT | | CCTGATACAAACTGCCGCCGA | |
| | | CTCCGGTTCACCCTTCAGCATCAATG | | CAGGTCACGGGTGTTGTGAAA | | TGGTACTTAACAGCGCCAGAC | |
| | | GTCGGGCTGGATGCTGGCGTGGCTA | | AATGTCACTGTCAGCCAGTCCT | | CTGCCAGCATCTGGATCCAAC | |
| | | AACTCGCCACGCTGTCAGATGGCAC | | GCGGTAAGTGGTACATCAGTA | | TTACGGGCAGGGCAGCCATC | |
| | | AGTCTTTGAGCCTGTAAACAGTTTTC | | TTCAGACAGAAAGTGAAGTAT | | ATCCCGGTAATGGCACTACCA | |
| | | AGAAAAACCAGAAGAAGCTGGCGAG | | CAACTCCGGTTCACCCTTCAGC | | AA | |
| | | ACTTCAGCGACAGTTAAGCCGCAAG | | ATCAATGGTCGGGCTGGATGC | | | |
| | | GTCAAATTCAGCAACAACTGGCAAA | | TGGCGTGGCTAAACTCGCCAC | | | |
| | | AGCAGAAACGCAAAATACAGCGACT | | GCTGTCAGATGGCACAGTCTTT | | | |
| | | GCATTCCTGTATCGCAAATATCCGCA | | GAGCCTGTAAACAGTTTTCAG | | | |
| | | GGGACTACCTTCACAAAGTCACAAC | | AAAAACCAGAAGAAGCTGGCG | | | |
| | | GGCCGTCAGCAAAAACCACGCAATG | | AGACTTCAGCGACAGTTAAGC | | | |
| | | ATAGTCATTGAGGATTTGAAGGTCAG | | CGCAAGGTCAAATTCAGCAAC | | | |
| | | CAACATGTCAAAGTCAGCAGCGGGT | | AACTGGCAAAAGCAGAAACGC | | | |
| | | ACGGTCAGCCAGCCGGGGCGCAATG | | AAAATACAGCGACTGCATTCC | | | |
| | | TCCGGGCAAAATCAGGTTTAAACCGT | | TGTATCGCAAATATCCGCAGG | | | |
| | | TCGATACTGGATCAGGGCTGGTATGA | | GACTACCTTCACAAAGTCACA | | | |
| | | AATGCGCCGCCAGCTTGCGTACAAG | | ACGGCCGTCAGCAAAAACCAC | | | |
| | | CAGCTCTGGCGTGGCGGTCAGGTGCT | | GCAATGATAGTCATTGAGGAT | | | |
| | | TGCTGTTCCGCCAGCGTATACAAGCC | | TTGAAGGTCAGCAACATGTCA | | | |
| | | AGCGTTGCGCGTACTGTGGTCATACA | | AAGTCAGCAGCGGGTACGGTC | | | |
| | | GCGAAAGAGAACCGCCTGTCACAAA | | AGCCAGCCGGGGCGCAATGTC | | | |
| | | GTAAATTCAGATGCCAGGTATGTGG | | CGGGCAAAATCAGGTTTAAAC | | | |
| | | ATATACAGCGAACGCCGATGTAAAT | | CGTTCGATACTGGATCAGGGC | | | |
| | | GGCGCTCGCAACATTTTAGCGGCGG | | TGGTATGAAATGCGCCGCCAG | | | |
| | | GGCACGCCGTTCTTGCCTGTGGAGAG | | CTTGCGTACAAGCAGCTCTGG | | | |
| | | ATGGTGCAGTCAGGCCGCCCGTTGA | | CGTGGCGGTCAGGTGCTTGCT | | | |
| | | AGCAGGAACCCACCGAAATGATTCA | | GTTCCGCCAGCGTATACAAGC | | | |
| | | GGCGACAGCCAGAACGTAGCAGGGA | | CAGCGTTGCGCGTACTGTGGT | | | |
| | | TCCACGTCCTTCAGGGCGTGGAGGAT | | CATACAGCGAAAGAGAACCGC | | | |
| | | GTCAATATCGGTTTCTGTCAGCGATT | | CTGTCACAAAGTAAATTCAGA | | | |
| | | AAATTCAACACCACGTAACAAACGC | | TGCCAGGTATGTGGATATACA | | | |
| | | CTCCGGCAATTAATCCCCAAAACGCA | | GCGAACGCCGATGTAAATGGC | | | |
| | | GAACCAATCCCGACCAGCGTCAATC | | GCTCGCAACATTTTAGCGGCG | | | |
| | | CACTTGCCGTTACCAGAAATGCCACC | | GGGCACGCCGTTCTTGCCTGTG | | | |
| | | ACCGCCGCGTCTCGCTCACGCTCATT | | GAGAGATGGTGCAGTCAGGCC | | | |
| | | ATGCAGCGCCTGATACAAACTGCCG | | GCCCGTTGAAGCAGGAACCCA | | | |
| | | CCGATGGTACTTAACAGCGCCAGAC | | CCGAAATGATTCAGGCGACAG | | | |
| | | CTGCCAGCATCTGGATCCAACTTACG | | CCAGAACGTAG | | | |
| | | GGCAGGGCAGCCATCATCCCGGTAA | | | | | |
| | | TGGCACTACCAAA | | | | | |
| 122 | YP_ | ATGAAAGAGGCAGTTAAAAATGTCA | 232 | ATGAAAGAGGCAGTTAAAAAT | 263 | GTTATAAGTATGGCACTGAAG | 294 |
| | 003986571.1 | AACCAAAAGTTCCCGCTAAAAAGAG | | GTCAAACCAAAAGTTCCCGCT | | TGGTTAATGTCAATGAATATT | |
| | | GATTATTACTGGAAGTAAAACTAAG | | AAAAAGAGGATTATTACTGGA | | TGACCACAAAAACCTGCTCAA | |
| | | AAAAAGGTTTTTGTCAAGAAGAAAC | | AGTAAAACTAAGAAAAAGGTT | | ACTGCGGCAAGATTAAAGAT | |
| | | CACCTGATAAGAAACCTCTTAAGAA | | TTTGTCAAGAAGAAACCACCT | | CTTGGAGCTAGCAAGATTTAC | |
| | | ACCCGTCAAAAAAACTGTTAAAACA | | GATAAGAAACCTCTTAAGAAA | | GAATGTGAGTCTTGTGGAATG | |
| | | GATAAACCAAAATCTATTTATGTTCC | | CCCGTCAAAAAAACTGTTAAA | | TACGCAGATCGTGATGAGAAT | |
| | | TAATAAAGATCTCAAAATATCCAAAT | | ACAGATAAACCAAAATCTATT | | GCTGCCAAGAATATCCTAAAA | |
| | | GGATTCCCACTCCCAAGAAAGAGTTT | | TATGTTCCTAATAAAGATCTCA | | GTAGGATTAAAGCCTTGGTAC | |
| | | ACTGAGATTGAAACTAATAGTTGGTA | | AAATATCCAAATGGATTCCCA | | AAACAAAAATAGTCTAATAA | |
| | | TGAACACAGGAAGTTTGAGAATCCT | | CTCCCAAGAAAGAGTTTACTG | | AATCAGGGGTACATTCCGCTA | |
| | | AATAAATCTCCTGTACAAACATACAA | | AGATTGAAACTAATAGTTGGT | | GTACTCCACCCTACGGGTTAA | |
| | | TAAGATTGTACCTGTAGTACCTCCTG | | ATGAACACAGGAAGTTTGAGA | | GCAAATGAGAATATCGAAAC | |
| | | AATCTATTAAACAACAGAATTTAGCA | | ATCCTAATAAATCTCCTGTACA | | GGTATGCACAGGATTCTTCGA | |
| | | AACAAACGTAAGAAAACAAATAGAC | | AACATACAATAAGATTGTACC | | GTGATAATCTTAGGATGACTC | |
| | | CTATTGTTTTTATTTCTTCTGAGAAAA | | TGTAGTACCTCCTGAATCTATT | | ACTAAGGAGATGACTAAAGT | |
| | | TCAGAATTTATCCCACCAAAGACCAG | | AAACAACAGAATTTAGCAAAC | | GTATCATTCAATATTGTATTG | |
| | | CAAAAAATTTTGCAAACTTGGTTTCG | | AAACGTAAGAAAACAAATAGA | | AACGGTATTCTTCCATAGAGA | |
| | | TTTATTTGCATATATGTATAACTGTA | | CCTATTGTTTTTATTTCTTCTGA | | GTTGATTTTTGGAGTATCCAA | |
| | | CCATTGATTACATTAATAGTAAGAAA | | GAAAATCAGAATTTATCCCAC | | AAATATCAACTTTTTATGAGC | |
| | | GTTGTTCTTGAATCTGGTAGAATTAA | | CAAAGACCAGCAAAAAATTTT | | GGATACATTAGAAACAGAAG | |
| | | TGTCGCTGCAACCAGGAAAGTTTGTA | | GCAAACTTGGTTTCGTTTATTT | | AGAAATACTGAAATATTTGCG | |
| | | ATAAAATTAGCGTTAGAAAAGCTCA | | GCATATATGTATAACTGTACC | | CCAGAAAGGATACGTTATCTA | |
| | | AAAAACTATTAGAGACAACTTAATC | | ATTGATTACATTAATAGTAAG | | GATTTTCAACTAAAAGGATTT | |
| | | CAATCCACTAATCCATCAATAATGAC | | AAAGTTGTTCTTGAATCTGGTA | | | |
| | | CCATATAATAGATGAAGCGATTGGTT | | GAATTAATGTCGCTGCAACCA | | | |
| | | TAGCTTGTTCAAATTATAAAACATGC | | GGAAAGTTTGTAATAAAATTA | | | |
| | | TTAACCAATTACATTGAAAGACATAT | | GCGTTAGAAAAGCTCAAAAAA | | | |
| | | TAAGAAATTTGATATTAAACCTTGGA | | CTATTAGAGACAACTTAATCC | | | |
| | | ATATGTCTAAAAGGAAGAAAATTAT | | AATCCACTAATCCATCAATAA | | | |
| | | CATTATAGAGGCTAATTTCTTTAAAA | | TGACCCATATAATAGATGAAG | | | |
| | | AAGGAACGTTTTGTCCCACAGTTTTC | | CGATTGGTTTAGCTTGTTCAAA | | | |
| | | CCTAAAATGGAATCTTCAAAACCACT | | TTATAAAACATGCTTAACCAA | | | |
| | | CACTATGATTGACAAGACTGTCACTT | | TTACATTGAAAGACATATTAA | | | |
| | | TGCAATATGATTCTGATACTAGAAAA | | GAAATTTGATATTAAACCTTG | | | |
| | | TATATCTTATTTGTACCTAGAGTCAC | | GAATATGTCTAAAAGGAAGAA | | | |
| | | TCCTAAGTATTCTGTCAATAAGGAAA | | AATTATCATTATAGAGGCTAA | | | |
| | | AAAATAGTTGTGGTATTGATCCTGGT | | TTTCTTTAAAAAAGGAACGTTT | | | |
| | | CTTAGAGATTTCCTTACGGTTTATTCT | | TGTCCCACAGTTTTCCCTAAAA | | | |
| | | GAAAATGAAACTCAATCAATTTGTCC | | TGGAATCTTCAAAACCACTCA | | | |
| | | TATTGAAATCGTAGTTAACACCACTA | | CTATGATTGACAAGACTGTCA | | | |
| | | AAAATGAATACAAGAAAATTGATAA | | CTTTGCAATATGATTCTGATAC | | | |
| | | GATCAACGAAATCATCAAAACTAAA | | TAGAAAATATATCTTATTTGTA | | | |
| | | CCAAATCTTAATAGTAAACGTAAAA | | CCTAGAGTCACTCCTAAGTATT | | | |
| | | AGAAACTTAATAGAGGCTTACGTAA | | CTGTCAATAAGGAAAAAAATA | | | |
| | | ATATCATAGAAGAGTTACTAATAAG | | GTTGTGGTATTGATCCTGGTCT | | | |
| | | ATGAAAGACATGCACTATAAAGTTTC | | TAGAGATTTCCTTACGGTTTAT | | | |
| | | TCATGAATTAGTAAATACTTTTGATA | | TCTGAAAATGAAACTCAATCA | | | |
| | | AAATTTGTATAGGTAAACTAAATGTA | | ATTTGTCCTATTGAAATCGTAG | | | |
| | | AAGTCTATCTTATCGAAAGCCAATAC | | TTAACACCACTAAAAATGAAT | | | |
| | | TGTTCTAAAAAGTGCCTTGAAAAGA | | ACAAGAAAATTGATAAGATCA | | | |
| | | AAACTTGCAACTCTGAGTTTTTATAG | | ACGAAATCATCAAAACTAAAC | | | |
| | | GTTTACTCAGAGATTAACTCATATGG | | CAAATCTTAATAGTAAACGTA | | | |
| | | GTTATAAGTATGGCACTGAAGTGGTT | | AAAAGAAACTTAATAGAGGCT | | | |
| | | AATGTCAATGAATATTTGACCACAAA | | TACGTAAATATCATAGAAGAG | | | |
| | | AACCTGCTCAAACTGCGGCAAGATT | | TTACTAATAAGATGAAAGACA | | | |
| | | AAAGATCTTGGAGCTAGCAAGATTT | | TGCACTATAAAGTTTCTCATGA | | | |
| | | ACGAATGTGAGTCTTGTGGAATGTAC | | ATTAGTAAATACTTTTGATAAA | | | |
| | | GCAGATCGTGATGAGAATGCTGCCA | | ATTTGTATAGGTAAACTAAAT | | | |
| | | AGAATATCCTAAAAGTAGGATTAAA | | GTAAAGTCTATCTTATCGAAA | | | |
| | | GCCTTGGTACAAACAAAAATAGTCT | | GCCAATACTGTTCTAAAAAGT | | | |
| | | AATAAAATCAGGGGTACATTCCGCT | | GCCTTGAAAAGAAAACTTGCA | | | |
| | | AGTACTCCACCCTACGGGTTAAGCAA | | ACTCTGAGTTTTTATAGGTTTA | | | |
| | | ATGAGAATATCGAAACGGTATGCAC | | CTCAGAGATTAACTCATATGG | | | |
| | | AGGATTCTTCGAGTGATAATCTTAGG | | GTTATAAGTATGGCACTGAAG | | | |
| | | ATGACTCACTAAGGAGATGACTAAA | | TGGTTAATGTCAATGAATATTT | | | |
| | | GTGTATCATTCAATATTGTATTGAAC | | GACCACAAAAACCTGCTCAAA | | | |
| | | GGTATTCTTCCATAGAGAGTTGATTT | | CTGCGGCAAGATTAAAGATCT | | | |
| | | TTGGAGTATCCAAAAATATCAACTTT | | TGGAGCTAGCAAGATTTACGA | | | |
| | | TTATGAGCGGATACATTAGAAACAG | | ATGTGAGTCTTGTGGAATGTA | | | |
| | | AAGAGAAATACTGAAATATTTGCGC | | CGCAGATCGTGATGAGAATGC | | | |
| | | CAGAAAGGATACGTTATCTAGATTTT | | TGCCAAGAATATCCTAAAAGT | | | |
| | | CAACTAAAAGGATTT | | AGGATTAAAGCCTTGGTACAA | | | |
| | | | | ACAAAAATAG | | | |
| 124 | WP_ | GTGAAGGTATACAAAAGGTTGTATG | 233 | GTGAAGGTATACAAAAGGTTG | 264 | TAAATGCGGACGTGAATGCG | 295 |
| | 010890547.1 | AACTCCTGGGCAGGGGATACAAACC | | TATGAACTCCTGGGCAGGGGA | | GTATTCAATCAACACTTTCTT | |
| | | GTTAAAGAGTGCAAGGTTAATAAAA | | TACAAACCGTTAAAGAGTGCA | | TACCTGTCCCATCTCCTAAAT | |
| | | CGTGGAAAAGATTACTACATAGGGA | | AGGTTAATAAAACGTGGAAAA | | GGAGGTGGGAAAGCCCGCCC | |
| | | TAACCTTGCAAAAAGCTGTCAAGGA | | GATTACTACATAGGGATAACC | | TGTGGTGCGGGTTGGGACTTC | |
| | | AAAGAAGATAAAAAAACCGAGGTAT | | TTGCAAAAAGCTGTCAAGGAA | | GCTGAAAAGTCCCTCACGTGA | |
| | | GTAATAAACGTGGATTTAAACGTTCA | | AAGAAGATAAAAAAACCGAG | | GGGACACAATCTAAGCGGAG | |
| | | GAGGAATTTAGCTTGCATAGGGATAT | | GTATGTAATAAACGTGGATTT | | TCCCTAAAGCAACAACTACCT | |
| | | TTGAAGTAGATTGGGAAAAAAGAGA | | AAACGTTCAGAGGAATTTAGC | | TTTTCTATATTTCTTCACTTTT | |
| | | AAGCAAGCTATACGGGATAAAGTTT | | TTGCATAGGGATATTTGAAGT | | GTCTACATTTTAAGGTAGCTG | |
| | | GTAAACGGAAAGCTTTTGAGACTTGT | | AGATTGGGAAAAAAGAGAAA | | TTGCAACCACTCATCAATAAC | |
| | | ATACAAAAGGGATTACTTGTTTGAGG | | GCAAGCTATACGGGATAAAGT | | TCTTCTAGTTTTCTAAGCATTC | |
| | | AGATAAGGAAGAAACAGAGGCAGAC | | TTGTAAACGGAAAGCTTTTGA | | CTTTACAACCGAGCGGTTTAT | |
| | | AGGAAGGAGTCCACAGGTAGGAGAT | | GACTTGTATACAAAAGGGATT | | CCAAAATAAGGCAAACACCC | |
| | | AATTCAAGACTGTGGAAAAAAGTAA | | ACTTGTTTGAGGAGATAAGGA | | TTCTATCAAGTTTCCCTTTATA | |
| | | ACAATTTAAACAGAGACATAGCTTTA | | AGAAACAGAGGCAGACAGGA | | GGTTATTCCCTTTTCAGAGAA | |
| | | AAAGTAGCGAAAGAAATAAGCGATA | | AGGAGTCCACAGGTAGGAGAT | | ATAGCTTTAGAGGGAAGTTTT | |
| | | TAGCAAGGGAGTTTTCAGAGAAAGG | | AATTCAAGACTGTGGAAAAAA | | CTTCCCTAAACATACGCTTCC | |
| | | AGAAGTTATAGTAGTATTTGAAAAA | | GTAAACAATTTAAACAGAGAC | | ATCTTCAGTTGTCCATTATCA | |
| | | CTAAAAGGACTCAGAGGTAGGAAAG | | ATAGCTTTAAAAGTAGCGAAA | | TGGACAACGAAATAGATAAA | |
| | | GAAGAAGCAAGAAGCTAAACAGAAA | | GAAATAAGCGATATAGCAAGG | | TAGCTTAGAAGATAAATATTT | |
| | | GATAAACTTCTGGATGAGAAGGAAA | | GAGTTTTCAGAGAAAGGAGAA | | GACAAAGTAAGCAGAAAAGT | |
| | | ATACAGGAGAGAGTAAAGGAACTCG | | GTTATAGTAGTATTTGAAAAA | | AGACATTTAGAATTATGACGG | |
| | | GATTAGAAGAAGGATTTGGATTAGA | | CTAAAAGGACTCAGAGGTAGG | | TTAAACAAGAAGACAAGCTT | |
| | | TTTTGTGTATCCGCATTATACGTCAA | | AAAGGAAGAAGCAAGAAGCT | | | |
| | | AGAAGTGTAGCAAATGCGGTTATGA | | AAACAGAAAGATAAACTTCTG | | | |
| | | GGGAGAGAGGTTCTCTCCCTCGGGTT | | GATGAGAAGGAAAATACAGG | | | |
| | | CAAAAGCGTTGTTTTTGTGTAAGAAG | | AGAGAGTAAAGGAACTCGGAT | | | |
| | | TGTGGATATGTGGTAAATGCGGACGT | | TAGAAGAAGGATTTGGATTAG | | | |
| | | GAATGCGGTATTCAATCAACACTTTC | | ATTTTGTGTATCCGCATTATAC | | | |
| | | TTTACCTGTCCCATCTCCTAAATGGA | | GTCAAAGAAGTGTAGCAAATG | | | |
| | | GGTGGGAAAGCCCGCCCTGTGGTGC | | CGGTTATGAGGGAGAGAGGTT | | | |
| | | GGGTTGGGACTTCGCTGAAAAGTCCC | | CTCTCCCTCGGGTTCAAAAGC | | | |
| | | TCACGTGAGGGACACAATCTAAGCG | | GTTGTTTTTGTGTAAGAAGTGT | | | |
| | | GAGTCCCTAAAGCAACAACTACCTTT | | GGATATGTGGTAAATGCGGAC | | | |
| | | TTCTATATTTCTTCACTTTTGTCTACA | | GTGAATGCGGTATTCAATCAA | | | |
| | | TTTTAAGGTAGCTGTTGCAACCACTC | | CACTTTCTTTACCTGTCCCATC | | | |
| | | ATCAATAACTCTTCTAGTTTTCTAAG | | TCCTAAATGGAGGTGGGAAAG | | | |
| | | CATTCCTTTACAACCGAGCGGTTTAT | | CCCGCCCTGTGGTGCGGGTTG | | | |
| | | CCAAAATAAGGCAAACACCCTTCTAT | | GGACTTCGCTGAAAAGTCCCT | | | |
| | | CAAGTTTCCCTTTATAGGTTATTCCCT | | CACGTGAGGGACACAATCTAA | | | |
| | | TTTCAGAGAAATAGCTTTAGAGGGA | | GCGGAGTCCCTAAAGCAACAA | | | |
| | | AGTTTTCTTCCCTAAACATACGCTTC | | CTACCTTTTTCTATATTTCTTCA | | | |
| | | CATCTTCAGTTGTCCATTATCATGGA | | CTTTTGTCTACATTTTAA | | | |
| | | CAACGAAATAGATAAATAGCTTAGA | | | | | |
| | | AGATAAATATTTGACAAAGTAAGCA | | | | | |
| | | GAAAAGTAGACATTTAGAATTATGA | | | | | |
| | | CGGTTAAACAAGAAGACAAGCTT | | | | | |
| 125 | YP_ | ATGAAAGAGGCAGTTAAAACTGTCA | 234 | ATGAAAGAGGCAGTTAAAACT | 265 | GTTATAAGTATGGCACTGAAG | 296 |
| | 003987385.1 | AACCAAAAGTTCCCGCTAAAAAGCG | | GTCAAACCAAAAGTTCCCGCT | | TGGTTAATGTCAATGAATATT | |
| | | GATTGTTACTGGTAGTAAAACTAAGA | | AAAAAGCGGATTGTTACTGGT | | TGACCACAAAAACCTGCTCAA | |
| | | AAAAGGTTTTTGTCAAGAAGAAACC | | AGTAAAACTAAGAAAAAGGTT | | ACTGCGGCAAGATTAAAGAT | |
| | | ACCGGCAAAAAAGCCACCTGATAAG | | TTTGTCAAGAAGAAACCACCG | | CTTGGAGCTAGCAAGATTTAC | |
| | | AAACCTCTTAAGAAAACTACCAAAA | | GCAAAAAAGCCACCTGATAAG | | GAATGTGAGTCTTGTGGAATG | |
| | | AAACAGTTAAAAAAGTGATAAATAG | | AAACCTCTTAAGAAAACTACC | | TACGCAGATCGTGATGAGAAT | |
| | | ACCCAAATCTATACATATTCCTAACA | | AAAAAAACAGTTAAAAAAGTG | | GCTGCCAAGAATATCCTAAAA | |
| | | AAGATCTCAAAATATTCAAATGGATT | | ATAAATAGACCCAAATCTATA | | GTAGGATTAAAGCCTTGGTAC | |
| | | CCCACTCCCAAGAAAGAGTTTACCG | | CATATTCCTAACAAAGATCTC | | AAACAAAAATAGTCTAATAA | |
| | | AGATTGAAACTAACAGTTGGTATGA | | AAAATATTCAAATGGATTCCC | | AATCAGGGGTACATTCCGCTA | |
| | | ACACAGGAAATTTGAGAATCCTAAT | | ACTCCCAAGAAAGAGTTTACC | | GTACTCCACCCTACGGGTTAA | |
| | | AAATCTCCTATACAAACATACAATAA | | GAGATTGAAACTAACAGTTGG | | GCAAATGAGAATATCGAAAC | |
| | | GATTGTACCTGTAGTTCCTCCGGAAT | | TATGAACACAGGAAATTTGAG | | GGTATGTATAGGATTCTTCAA | |
| | | CCATTAAACAACAGAATTTAGCAAA | | AATCCTAATAAATCTCCTATAC | | GTAGTAATCCCAGGATGACTC | |
| | | CAAACGTAAGAAAACAAATAGACCT | | AAACATACAATAAGATTGTAC | | ACTAAGGAGATGACTAAAGT | |
| | | ATTGTTTTTATTTCTTCTGAGAAAATC | | CTGTAGTTCCTCCGGAATCCAT | | GTATCATTCAATACTTCATTG | |
| | | AGAATTTATCCCACCAAAGAGCAAC | | TAAACAACAGAATTTAGCAAA | | AACGGTATTCTTTCATAGAGA | |
| | | AAAAAATTTTACAAACTTGGTTTCGT | | CAAACGTAAGAAAACAAATAG | | ATTGATTTCTGGAGTATCCAG | |
| | | TTATTTGCATGTATGTATAATAGTTC | | ACCTATTGTTTTTATTTCTTCTG | | AAATATCAACTTTTTATGAGC | |
| | | TATTGATTACATTAACAGTAAGAAAG | | AGAAAATCAGAATTTATCCCA | | GGTATATACATCACATTATTA | |
| | | TTGTTCTTGAATCTGGTAGAATTAAT | | CCAAAGAGCAACAAAAAATTT | | GACTAATGATTATTTGTAATT | |
| | | GTCGCTGCAACCAGGAAAGTTTGTA | | TACAAACTTGGTTTCGTTTATT | | TCATGAATATTTGTATGATAG | |
| | | ATAAAATTAGCGTTAGAAAAGCTCT | | TGCATGTATGTATAATAGTTCT | | ATCCGAATGATGTTTATAAAT | |
| | | AAAAACTATTAGAGACAATTTAATC | | ATTGATTACATTAACAGTAAG | | | |
| | | AAATCCACTAATCCATCAATAATGAC | | AAAGTTGTTCTTGAATCTGGTA | | | |
| | | CCATATAATGGACGAAGCGATTGGTT | | GAATTAATGTCGCTGCAACCA | | | |
| | | TAGCTTGTTCAAATTATAAGACATGT | | GGAAAGTTTGTAATAAAATTA | | | |
| | | TTAACCAATTATATTGAAGGTCAAAT | | GCGTTAGAAAAGCTCTAAAAA | | | |
| | | TAAAAAATTTGATATTAAACCTTGGT | | CTATTAGAGACAATTTAATCA | | | |
| | | CTATCTCTAAAAGACGAAAGATTATT | | AATCCACTAATCCATCAATAA | | | |
| | | GTCATTGAACCTGGATACTTTAAAGG | | TGACCCATATAATGGACGAAG | | | |
| | | AAATTCTTTTTGTCCCACAGTTTTTCC | | CGATTGGTTTAGCTTGTTCAAA | | | |
| | | CAAAATGAAATCTTCTAAACCACTTA | | TTATAAGACATGTTTAACCAAT | | | |
| | | TTATGATTGATAAAACTGTGACTTTG | | TATATTGAAGGTCAAATTAAA | | | |
| | | CAATACGATTCTGATACTAGAAAGTA | | AAATTTGATATTAAACCTTGGT | | | |
| | | CATCTTATTTGTACCTAGAGTCACTC | | CTATCTCTAAAAGACGAAAGA | | | |
| | | CTAAGTATTCTGTCAATAAGGAAAA | | TTATTGTCATTGAACCTGGATA | | | |
| | | AAATAGTTGTGGTATTGATCCTGGTC | | CTTTAAAGGAAATTCTTTTTGT | | | |
| | | TTAGAGATTTCCTTACGGTTTATTCT | | CCCACAGTTTTTCCCAAAATGA | | | |
| | | GAAAATGAAACTCAATCAATTTGTCC | | AATCTTCTAAACCACTTATTAT | | | |
| | | TATTGAAATCGTAGTTAACACCACTA | | GATTGATAAAACTGTGACTTT | | | |
| | | AAAATGAATACAAGAAAATTGATAA | | GCAATACGATTCTGATACTAG | | | |
| | | GATCAACGAAATCATCAAAACTAAA | | AAAGTACATCTTATTTGTACCT | | | |
| | | CCAAATCTTAATAGTAAACGTAAAA | | AGAGTCACTCCTAAGTATTCTG | | | |
| | | AGAAACTTAATAGAGGCTTACGTAA | | TCAATAAGGAAAAAAATAGTT | | | |
| | | ATATCATAGAAGAGTTACTAATAAG | | GTGGTATTGATCCTGGTCTTAG | | | |
| | | ATGAAAGACATGCACTATAAAGTTTC | | AGATTTCCTTACGGTTTATTCT | | | |
| | | TCATGAATTAGTAAATACTTTTGATA | | GAAAATGAAACTCAATCAATT | | | |
| | | AAATTTGTATAGGTAAACTAAATGTA | | TGTCCTATTGAAATCGTAGTTA | | | |
| | | AAGTCTATCTTATCGAAAGCCAATAC | | ACACCACTAAAAATGAATACA | | | |
| | | TGTTCTAAAAAGTGCCTTGAAAAGA | | AGAAAATTGATAAGATCAACG | | | |
| | | AAACTTGCAACTCTGAGTTTTTATAG | | AAATCATCAAAACTAAACCAA | | | |
| | | GTTTACTCAGAGATTAACTCATATGG | | ATCTTAATAGTAAACGTAAAA | | | |
| | | GTTATAAGTATGGCACTGAAGTGGTT | | AGAAACTTAATAGAGGCTTAC | | | |
| | | AATGTCAATGAATATTTGACCACAAA | | GTAAATATCATAGAAGAGTTA | | | |
| | | AACCTGCTCAAACTGCGGCAAGATT | | CTAATAAGATGAAAGACATGC | | | |
| | | AAAGATCTTGGAGCTAGCAAGATTT | | ACTATAAAGTTTCTCATGAATT | | | |
| | | ACGAATGTGAGTCTTGTGGAATGTAC | | AGTAAATACTTTTGATAAAATT | | | |
| | | GCAGATCGTGATGAGAATGCTGCCA | | TGTATAGGTAAACTAAATGTA | | | |
| | | AGAATATCCTAAAAGTAGGATTAAA | | AAGTCTATCTTATCGAAAGCC | | | |
| | | GCCTTGGTACAAACAAAAATAGTCT | | AATACTGTTCTAAAAAGTGCC | | | |
| | | AATAAAATCAGGGGTACATTCCGCT | | TTGAAAAGAAAACTTGCAACT | | | |
| | | AGTACTCCACCCTACGGGTTAAGCAA | | CTGAGTTTTTATAGGTTTACTC | | | |
| | | ATGAGAATATCGAAACGGTATGTAT | | AGAGATTAACTCATATGGGTT | | | |
| | | AGGATTCTTCAAGTAGTAATCCCAGG | | ATAAGTATGGCACTGAAGTGG | | | |
| | | ATGACTCACTAAGGAGATGACTAAA | | TTAATGTCAATGAATATTTGAC | | | |
| | | GTGTATCATTCAATACTTCATTGAAC | | CACAAAAACCTGCTCAAACTG | | | |
| | | GGTATTCTTTCATAGAGAATTGATTT | | CGGCAAGATTAAAGATCTTGG | | | |
| | | CTGGAGTATCCAGAAATATCAACTTT | | AGCTAGCAAGATTTACGAATG | | | |
| | | TTATGAGCGGTATATACATCACATTA | | TGAGTCTTGTGGAATGTACGC | | | |
| | | TTAGACTAATGATTATTTGTAATTTC | | AGATCGTGATGAGAATGCTGC | | | |
| | | ATGAATATTTGTATGATAGATCCGAA | | CAAGAATATCCTAAAAGTAGG | | | |
| | | TGATGTTTATAAAT | | ATTAAAGCCTTGGTACAAACA | | | |
| | | | | AAAATAG | | | |
| 126 | WP_ | ATGAAGAGAATGAGCAGTTTAGCCT | 235 | ATGAAGAGAATGAGCAGTTTA | 266 | CTAAGTACACCACCCAAAGAT | 297 |
| | 163596789.1 | ACCATTTTGGCTTGAAAGTACGCTTT | | GCCTACCATTTTGGCTTGAAAG | | GCCATCAATGTGGCAGTATTA | |
| | | TATCCTAGTTCTAAACAAAAGAAAAT | | TACGCTTTTATCCTAGTTCTAA | | TGGGACAAAACGGTTATAAG | |
| | | CATCAAGCTAAATTATGATGCTCAGC | | ACAAAAGAAAATCATCAAGCT | | AAATTAACGCTTAAGGATCGA | |
| | | GTTTTGTCTACAACTCTTATGTAGGA | | AAATTATGATGCTCAGCGTTTT | | GAATGGACTTGTCCAATCTGT | |
| | | CGTAATCGGAACAATTATCGGGCTA | | GTCTACAACTCTTATGTAGGAC | | CAAACGCATCATATCCGTGAT | |
| | | GGGGCTACTTAGCTGTTAGACAATAT | | GTAATCGGAACAATTATCGGG | | TGGAATGCGGCAGTAAACAT | |
| | | CAAGTAATGCCTTTTGCCTTTTCTGTT | | CTAGGGGCTACTTAGCTGTTA | | CTTAGAAAAAGGCTTAGGCA | |
| | | CTTAATAATTATGAGACTAAGCTAGC | | GACAATATCAAGTAATGCCTT | | ACTGGCAGAATTCTAAAATAA | |
| | | AGAAGAAGTAGTCGCCAATAATGAG | | TTGCCTTTTCTGTTCTTAATAA | | AAAAAGCAGCCTAAGCTAGG | |
| | | TTATTGGCTAAGCCAAAGAACATTCG | | TTATGAGACTAAGCTAGCAGA | | CTGCAGTCCGCTAGCAACTCG | |
| | | CGATGCTTATAGCTTTTTACGCGTTA | | AGAAGTAGTCGCCAATAATGA | | CGGACTCAAAAGGCTTTGGTA | |
| | | AAGAAATTGACAGTCTCGCTATTGCC | | GTTATTGGCTAAGCCAAAGAA | | ATTAGACGACCTCTATCGGCA | |
| | | AATGCGATTCAAAACTATCAGAAAG | | CATTCGCGATGCTTATAGCTTT | | ATAGTCAAAAGATTATTGTTG | |
| | | CTTGGAACAACTATCGCAAGCTTGGT | | TTACGCGTTAAAGAAATTGAC | | GTAAGTCAAGTCCGTATCTAA | |
| | | CATGGAATTCCGACTTTTCACAAAAA | | AGTCTCGCTATTGCCAATGCG | | GCAAGTTGGTGTTCTGGTTAG | |
| | | ACGTAGTGATTGGTCATATCAGACCA | | ATTCAAAACTATCAGAAAGCT | | CGCAAGCTAACGAGCCAGCC | |
| | | ACTGTCAGTATCCTAAACAGACAGA | | TGGAACAACTATCGCAAGCTT | | ACCAAGCCCCAGAATTTATTC | |
| | | AGCCTTTCTTGATAACGGGACAGCTA | | GGTCATGGAATTCCGACTTTTC | | TGGGGTAGTTGACTTATCTTG | |
| | | GATTTATTGATGCTAAGCACGTTAAA | | ACAAAAAACGTAGTGATTGGT | | AGTCTATTTACGGGAAGCTAT | |
| | | TTGCCTAAGCTAGGCGTTGTCCGCAT | | CATATCAGACCAACTGTCAGT | | TCAATTCGGCAGATTAATCCG | |
| | | TGCCAGCTTTAGAAAATTGATTAAAG | | ATCCTAAACAGACAGAAGCCT | | CTTGAAAAAGTGAAGTTCAA | |
| | | AGCGTTTTCTTAATCATATTCCAACT | | TTCTTGATAACGGGACAGCTA | | GTATGTGCAATGGCTATCTTT | |
| | | AGAATTGGCACAGTCACGATTAAAA | | GATTTATTGATGCTAAGCACGT | | GGCTATTGGGCTGGTCTGTTT | |
| | | AGACCGCTGACGACCAGTTTTACTTA | | TAAATTGCCTAAGCTAGGCGT | | AA | |
| | | TCAATGCAACTAGGCAGCGATATTGC | | TGTCCGCATTGCCAGCTTTAGA | | | |
| | | CTTTGTTAAGGAATTGCCTAAGACGC | | AAATTGATTAAAGAGCGTTTT | | | |
| | | AGAGTCAAATTGGCATCGACCTCAAT | | CTTAATCATATTCCAACTAGAA | | | |
| | | CTTGATAATTTCTTAACAGCATCTAA | | TTGGCACAGTCACGATTAAAA | | | |
| | | CGGAGCAATGGTGGCTAATCCACGA | | AGACCGCTGACGACCAGTTTT | | | |
| | | TTTTATCGCAAAACCAAAAAGAAGC | | ACTTATCAATGCAACTAGGCA | | | |
| | | TTGCCCATGCCCAACGTGTCTTGTCT | | GCGATATTGCCTTTGTTAAGGA | | | |
| | | CGCAGGCAGCGCAGAGCTAAGAAAG | | ATTGCCTAAGACGCAGAGTCA | | | |
| | | AGGGACGCAATTTGCGCTTAGCCAA | | AATTGGCATCGACCTCAATCTT | | | |
| | | GAATTATCAAAAACAGCGTCTAGCA | | GATAATTTCTTAACAGCATCTA | | | |
| | | GTCGCTAAACTGCACGATAAGATTCG | | ACGGAGCAATGGTGGCTAATC | | | |
| | | CAGACAGCGAAATGACTTCTTGCAA | | CACGATTTTATCGCAAAACCA | | | |
| | | GTACTCTCAACTGCACTAATCAAAAA | | AAAAGAAGCTTGCCCATGCCC | | | |
| | | CCACGATTTAGTAGTTGCCGAGGAAT | | AACGTGTCTTGTCTCGCAGGC | | | |
| | | TAAGAAGCAAGAACCTGTTAAAGAA | | AGCGCAGAGCTAAGAAAGAG | | | |
| | | TCATGCCTTGTCTCAAGCTATTTCTG | | GGACGCAATTTGCGCTTAGCC | | | |
| | | ATGTTGGCTGGCGCAGTTTTCTGAAC | | AAGAATTATCAAAAACAGCGT | | | |
| | | ATGCTGGCATATAAGGCAGACCTAT | | CTAGCAGTCGCTAAACTGCAC | | | |
| | | ATGGCAAAGAGTTCAAAACAATTGA | | GATAAGATTCGCAGACAGCGA | | | |
| | | TCCTAAGTACACCACCCAAAGATGCC | | AATGACTTCTTGCAAGTACTCT | | | |
| | | ATCAATGTGGCAGTATTATGGGACA | | CAACTGCACTAATCAAAAACC | | | |
| | | AAACGGTTATAAGAAATTAACGCTT | | ACGATTTAGTAGTTGCCGAGG | | | |
| | | AAGGATCGAGAATGGACTTGTCCAA | | AATTAAGAAGCAAGAACCTGT | | | |
| | | TCTGTCAAACGCATCATATCCGTGAT | | TAAAGAATCATGCCTTGTCTCA | | | |
| | | TGGAATGCGGCAGTAAACATCTTAG | | AGCTATTTCTGATGTTGGCTGG | | | |
| | | AAAAAGGCTTAGGCAACTGGCAGAA | | CGCAGTTTTCTGAACATGCTGG | | | |
| | | TTCTAAAATAAAAAAAGCAGCCTAA | | CATATAAGGCAGACCTATATG | | | |
| | | GCTAGGCTGCAGTCCGCTAGCAACTC | | GCAAAGAGTTCAAAACAATTG | | | |
| | | GCGGACTCAAAAGGCTTTGGTAATTA | | ATCCTAAGTACACCACCCAAA | | | |
| | | GACGACCTCTATCGGCAATAGTCAA | | GATGCCATCAATGTGGCAGTA | | | |
| | | AAGATTATTGTTGGTAAGTCAAGTCC | | TTATGGGACAAAACGGTTATA | | | |
| | | GTATCTAAGCAAGTTGGTGTTCTGGT | | AGAAATTAACGCTTAAGGATC | | | |
| | | TAGCGCAAGCTAACGAGCCAGCCAC | | GAGAATGGACTTGTCCAATCT | | | |
| | | CAAGCCCCAGAATTTATTCTGGGGTA | | GTCAAACGCATCATATCCGTG | | | |
| | | GTTGACTTATCTTGAGTCTATTTACG | | ATTGGAATGCGGCAGTAAACA | | | |
| | | GGAAGCTATTCAATTCGGCAGATTAA | | TCTTAGAAAAAGGCTTAGGCA | | | |
| | | TCCGCTTGAAAAAGTGAAGTTCAAGT | | ACTGGCAGAATTCTAAAATAA | | | |
| | | ATGTGCAATGGCTATCTTTGGCTATT | | AAAAAGCAGCCTAA | | | |
| | | GGGCTGGTCTGTTTAA | | | | | |
| 128 | WP_ | ATGAAATCTTCCATGGCTATGATAAA | 236 | ATGAAATCTTCCATGGCTATG | 267 | AGGAAAGGACATCACTCATC | 298 |
| | 010992545.1 | ACATAAAAAGGAGCTGGTTGAATAT | | ATAAAACATAAAAAGGAGCTG | | ATCACGGCAAACAAGGCACT | |
| | | GCACGGCACCTGAAACTACCGAACT | | GTTGAATATGCACGGCACCTG | | CACCCGTTGGCTGGAAACATT | |
| | | TGGCAGAACATGTGGGTGTGATACTT | | AAACTACCGAACTTGGCAGAA | | GGAGGATGAAGCGGTCACAG | |
| | | CATGAAGCACAGGAGAAGCAACTGA | | CATGTGGGTGTGATACTTCATG | | CCGCCTTGCTTGACAGGCTGC | |
| | | CCTATTCGGAGTTCCTGGCGAATTGT | | AAGCACAGGAGAAGCAACTGA | | TCTACTGCTGCGAGATTATCA | |
| | | CTTGCCAGGGAAATCCAGGGCAGAG | | CCTATTCGGAGTTCCTGGCGA | | GGCTCGGAGGAACAAGCTAT | |
| | | AAAGGAAAAGCTACCTGACACGGTT | | ATTGTCTTGCCAGGGAAATCC | | CGCATGCAAAACAGGAAAAC | |
| | | GAAACTCTCCGGACTCCCCGCCAAAT | | AGGGCAGAGAAAGGAAAAGC | | AATTTTTAGCAACCAAAACAC | |
| | | ACGATCTGGACCTGTACGACTATGAC | | TACCTGACACGGTTGAAACTC | | GGATATAGGCACGTAAAAAG | |
| | | AGGGCGGAAGGAATGGATAACAGGA | | TCCGGACTCCCCGCCAAATAC | | AGTTAAGGAAAGTGAAGCAT | |
| | | GGCTGCGCGAACTGCGTGAGCTGGT | | GATCTGGACCTGTACGACTAT | | CTTCGATGCTGGAGTGGGATA | |
| | | ATGGGTGAACCAGGCATACAATCTTC | | GACAGGGCGGAAGGAATGGAT | | TACTAAATTACCTAAAAAAGT | |
| | | TCCTGGTAGGCCCTTCCGGAACCGGC | | AACAGGAGGCTGCGCGAACTG | | GGCGCACAAATTTGCGCGCCA | |
| | | AAGACCTTTATAGCCGCCGGACTCGT | | CGTGAGCTGGTATGGGTGAAC | | CAATAATGGCAATTTATTAAT | |
| | | GTATGAAGCCGTAAAAGCGGGATAC | | CAGGCATACAATCTTCTCCTGG | | CAATAGATGGACTGAATCACC | |
| | | GAAGCATATCTGATGACACTGGAGG | | TAGGCCCTTCCGGAACCGGCA | | TATGACAGTATCTCATTTTAA | |
| | | AGCTGCTCACATGCCTGAAAACGAA | | AGACCTTTATAGCCGCCGGAC | | TGATCTACTAATAACTTGTAA | |
| | | AGAGGTCTCCGCACATGCGATGAAA | | TCGTGTATGAAGCCGTAAAAG | | AGGTACTATTGGTGAAATGGC | |
| | | ACATACAAGCGGATCATGAAGGCCC | | CGGGATACGAAGCATATCTGA | | TTTTAATAGAATCGGTAGTAT | |
| | | GGCTGCTGGCCATAGACGATGCCAC | | TGACACTGGAGGAGCTGCTCA | | TCACATAGCTCGCCAAATAAT | |
| | | ACTGTTCCCTTTGAAAAGAGAAGAA | | CATGCCTGAAAACGAAAGAGG | | GGCATTAACTGGCATTGTTGG | |
| | | GCCGTGTTGCTGTTCAAACTGGTCAA | | TCTCCGCACATGCGATGAAAA | | TGACTTGGATTATATTAGATG | |
| | | TGACTTCCAGGAAAGGACATCACTC | | CATACAAGCGGATCATGAAGG | | GTTCCTTTATGCCAATGTATC | |
| | | ATCATCACGGCAAACAAGGCACTCA | | CCCGGCTGCTGGCCATAGACG | | ATC | |
| | | CCCGTTGGCTGGAAACATTGGAGGA | | ATGCCACACTGTTCCCTTTGAA | | | |
| | | TGAAGCGGTCACAGCCGCCTTGCTTG | | AAGAGAAGAAGCCGTGTTGCT | | | |
| | | ACAGGCTGCTCTACTGCTGCGAGATT | | GTTCAAACTGGTCAATGACTTC | | | |
| | | ATCAGGCTCGGAGGAACAAGCTATC | | CAGGAAAGGACATCACTCATC | | | |
| | | GCATGCAAAACAGGAAAACAATTTT | | ATCACGGCAAACAAGGCACTC | | | |
| | | TAGCAACCAAAACACGGATATAGGC | | ACCCGTTGGCTGGAAACATTG | | | |
| | | ACGTAAAAAGAGTTAAGGAAAGTGA | | GAGGATGAAGCGGTCACAGCC | | | |
| | | AGCATCTTCGATGCTGGAGTGGGATA | | GCCTTGCTTGACAGGCTGCTCT | | | |
| | | TACTAAATTACCTAAAAAAGTGGCG | | ACTGCTGCGAGATTATCAGGC | | | |
| | | CACAAATTTGCGCGCCACAATAATG | | TCGGAGGAACAAGCTATCGCA | | | |
| | | GCAATTTATTAATCAATAGATGGACT | | TGCAAAACAGGAAAACAATTT | | | |
| | | GAATCACCTATGACAGTATCTCATTT | | TTAGCAACCAAAACACGGATA | | | |
| | | TAATGATCTACTAATAACTTGTAAAG | | TAGGCACGTAA | | | |
| | | GTACTATTGGTGAAATGGCTTTTAAT | | | | | |
| | | AGAATCGGTAGTATTCACATAGCTCG | | | | | |
| | | CCAAATAATGGCATTAACTGGCATTG | | | | | |
| | | TTGGTGACTTGGATTATATTAGATGG | | | | | |
| | | TTCCTTTATGCCAATGTATCATC | | | | | |
| 133 | WP_ | ATGAATGCTTCTAGTAAAAGGAAAA | 237 | ATGAATGCTTCTAGTAAAAGG | 268 | GTTGTCCACATGCGAATGCAT | 299 |
| | 001557544.1 | TTATTAGTCAGAGTGAGATTAGCAAA | | AAAATTATTAGTCAGAGTGAG | | GCTTAGATTGCACTCACTTCT | |
| | | AAAATAGCTGTAATGAATGAAGAAA | | ATTAGCAAAAAAATAGCTGTA | | GTACCAGTAAGCAATTCTTAC | |
| | | TGCAGGGGTTTTGGGCTAATAATAGT | | ATGAATGAAGAAATGCAGGGG | | CACAGCACGAAGAACAGTTA | |
| | | TGGGATATAAGAAAATGTCCACATC | | TTTTGGGCTAATAATAGTTGGG | | GAGCGTACAGAAGAGTTATT | |
| | | CTTCTGCCATAGAATTAAGTAAGAAT | | ATATAAGAAAATGTCCACATC | | AGCCATAGCTAAGGATAAAC | |
| | | CCTGCTTTAAGGAATCGTTGGGTTCG | | CTTCTGCCATAGAATTAAGTA | | AATGGCAAAGACAAGTAGAG | |
| | | TTTTGAACGTGTTAAAAATCTGTGGT | | AGAATCCTGCTTTAAGGAATC | | ACTAATAGCCGTGTTAAAGAG | |
| | | TAAGAACAGAATTGAAATATTTTTAT | | GTTGGGTTCGTTTTGAACGTGT | | CGTTTAGAACAAATCATTGGA | |
| | | TTTTACCATTTAAACAATGGAATATG | | TAAAAATCTGTGGTTAAGAAC | | AGTTTGACGGGGTAATTATCA | |
| | | GAATGCAAAAACTGTCTGGATTAGA | | AGAATTGAAATATTTTTATTTT | | ATGGATAAACAAGTTAGAAA | |
| | | AAAGGAACAGTAATTAATAAAATGT | | TACCATTTAAACAATGGAATA | | TACAACAGAAATTGTACGTTT | |
| | | TAGATTTCTTAGATTTAAAGTATCCT | | TGGAATGCAAAAACTGTCTGG | | GGCGAAGCAGAAATCAAAAA | |
| | | AGCATTACTTCAATTACTGAAGTTCC | | ATTAGAAAAGGAACAGTAATT | | AGACAAGGGAAAAAGTAGAC | |
| | | TATTGAAAAAGCAATGACGGAGTAT | | AATAAAATGTTAGATTTCTTAG | | AAAGCGATTTCTAAATTTTCG | |
| | | AGAACTTATTTGACAAAACGGGGTG | | ATTTAAAGTATCCTAGCATTAC | | ATTGAAGGTAAAGTTATTAAT | |
| | | TTAGAATTACCACTACTAATTATAAG | | TTCAATTACTGAAGTTCCTATT | | TTTAATTCAATAGCAAAGGAA | |
| | | ATTACTGCTAATCAAGAAAAAACAC | | GAAAAAGCAATGACGGAGTAT | | GCTAATGTTTCTAAATCATGG | |
| | | CTGTAAAAGCTAATTCCTACTATGTT | | AGAACTTATTTGACAAAACGG | | CTTTATAAGGAACACGATATT | |
| | | ACTAATCTAAAACAATTTATGGAGTT | | GGTGTTAGAATTACCACTACT | | AGGCAAAGAATCGAATCCCTT | |
| | | TTATGAGAACTTTTATTTTGATGGAG | | AATTATAAGATTACTGCTAATC | | CGTGAGCGTCAAATAACAGC | |
| | | AGGAGTGGGATAAAGACGTTTGGGA | | AAGAAAAAACACCTGTAAAAG | | AAATGTAGTCTCAAAACCCAA | |
| | | TAGACGTAACTTACCTTTGCCAGATG | | CTAATTCCTACTATGTTACTAA | | GAAAAGTTCTCGTTCGGAGGA | |
| | | ATAAGGTTAACCCAACACAATATGA | | TCTAAAACAATTTATGGAGTTT | | AATCCTTATTAAAACCTTAAA | |
| | | ATATACAATTAACTTTAAAGGGTTTC | | TATGAGAACTTTTATTTTGATG | | AAG | |
| | | GGAATACATATTTTAAACAACTTGTA | | GAGAGGAGTGGGATAAAGAC | | | |
| | | AAAAGATATTGTAAGTTGAGATTGA | | GTTTGGGATAGACGTAACTTA | | | |
| | | ACGTGGATAGCTTTTCCTATGTAAGT | | CCTTTGCCAGATGATAAGGTT | | | |
| | | GATATTGCCCAAAGACTTAAAGAGTT | | AACCCAACACAATATGAATAT | | | |
| | | CTTTAATTTTCTGGACATGAAATTTA | | ACAATTAACTTTAAAGGGTTTC | | | |
| | | AACAAGTTCAGAGAGTACACCAATT | | GGAATACATATTTTAAACAAC | | | |
| | | AACGAGAGTGGAAATTGAAGCATAT | | TTGTAAAAAGATATTGTAAGT | | | |
| | | TTAAGTGAACTAAACATGATGGGAA | | TGAGATTGAACGTGGATAGCT | | | |
| | | TAAAACCTAGTACAATAACTGGGAG | | TTTCCTATGTAAGTGATATTGC | | | |
| | | GATCTCTATATTGGAAGGACTATTTA | | CCAAAGACTTAAAGAGTTCTT | | | |
| | | GTACCCTTCTTAGGCTAGAATGGGAT | | TAATTTTCTGGACATGAAATTT | | | |
| | | GATGTTCCTTCCAAAATATTAATTTA | | AAACAAGTTCAGAGAGTACAC | | | |
| | | TTCTGAGGACTATCCGAAAATACCAA | | CAATTAACGAGAGTGGAAATT | | | |
| | | GAGCAAAACCACGCTTTATAGATGA | | GAAGCATATTTAAGTGAACTA | | | |
| | | ATTCGTCCTAGAGCAATTGAACAGTC | | AACATGATGGGAATAAAACCT | | | |
| | | ATCTTGATAAATTACCCGAATATATA | | AGTACAATAACTGGGAGGATC | | | |
| | | GCTACGATGACTATGATTGTTCAAGA | | TCTATATTGGAAGGACTATTTA | | | |
| | | ATGTGGAATGAGGATAAGTGAATTG | | GTACCCTTCTTAGGCTAGAATG | | | |
| | | TGCACCTTGAAAAAAGGCTGTCTATT | | GGATGATGTTCCTTCCAAAAT | | | |
| | | AGAGGACAAAGATGGAGATTTCTTTT | | ATTAATTTATTCTGAGGACTAT | | | |
| | | TAAAGTATTATCAATGGAAAATGAA | | CCGAAAATACCAAGAGCAAAA | | | |
| | | AAAGGAGCATATAGTTCCAATATCTA | | CCACGCTTTATAGATGAATTCG | | | |
| | | AAGAGGTAGCTTTACTTATTAAAGTT | | TCCTAGAGCAATTGAACAGTC | | | |
| | | CGGGAAGATAAAGTTTCAGAGGAAT | | ATCTTGATAAATTACCCGAAT | | | |
| | | TTCCAGATAGTGAATACCTCTTTCCA | | ATATAGCTACGATGACTATGA | | | |
| | | AGAAAAGATGGATCGCCATTAAAAC | | TTGTTCAAGAATGTGGAATGA | | | |
| | | AAGAAACATTTAGAGGTGAGTTAAA | | GGATAAGTGAATTGTGCACCT | | | |
| | | TAAATTAGCTTATGAGCAAAATATAG | | TGAAAAAAGGCTGTCTATTAG | | | |
| | | TGGATAAATCAGGTGAGATTTATAG | | AGGACAAAGATGGAGATTTCT | | | |
| | | ATTCCATGCCCATGCCTTTCGCCATA | | TTTTAAAGTATTATCAATGGAA | | | |
| | | CAGTAGGAACAAGAATGATTAACAA | | AATGAAAAAGGAGCATATAGT | | | |
| | | CGGGATGCCCCAGCATATTGTGCAG | | TCCAATATCTAAAGAGGTAGC | | | |
| | | AAATTTTTGGGGCATGAAAGCCCAG | | TTTACTTATTAAAGTTCGGGAA | | | |
| | | AAATGACAAGCAGATACGCTCATAT | | GATAAAGTTTCAGAGGAATTT | | | |
| | | CTTTGATGAAACTCTAAAAAATGAAT | | CCAGATAGTGAATACCTCTTTC | | | |
| | | TTACTAAATTTCAGGAAAAACTGGTT | | CAAGAAAAGATGGATCGCCAT | | | |
| | | ACCAATAATGGAGATGTGCTTGATCT | | TAAAACAAGAAACATTTAGAG | | | |
| | | AGATGAAGATAATGAAGTCGATGAT | | GTGAGTTAAATAAATTAGCTT | | | |
| | | GTAGAGCTTCAATGGTTCAAGAAAA | | ATGAGCAAAATATAGTGGATA | | | |
| | | ATATAAATGCACAAGTGCTTCCAAAT | | AATCAGGTGAGATTTATAGAT | | | |
| | | GGTTATTGTAGATTGCCAGTAGTAGC | | TCCATGCCCATGCCTTTCGCCA | | | |
| | | AGGTGGTTGTCCACATGCGAATGCAT | | TACAGTAGGAACAAGAATGAT | | | |
| | | GCTTAGATTGCACTCACTTCTGTACC | | TAACAACGGGATGCCCCAGCA | | | |
| | | AGTAAGCAATTCTTACCACAGCACG | | TATTGTGCAGAAATTTTTGGGG | | | |
| | | AAGAACAGTTAGAGCGTACAGAAGA | | CATGAAAGCCCAGAAATGACA | | | |
| | | GTTATTAGCCATAGCTAAGGATAAAC | | AGCAGATACGCTCATATCTTTG | | | |
| | | AATGGCAAAGACAAGTAGAGACTAA | | ATGAAACTCTAAAAAATGAAT | | | |
| | | TAGCCGTGTTAAAGAGCGTTTAGAAC | | TTACTAAATTTCAGGAAAAAC | | | |
| | | AAATCATTGGAAGTTTGACGGGGTA | | TGGTTACCAATAATGGAGATG | | | |
| | | ATTATCAATGGATAAACAAGTTAGA | | TGCTTGATCTAGATGAAGATA | | | |
| | | AATACAACAGAAATTGTACGTTTGGC | | ATGAAGTCGATGATGTAGAGC | | | |
| | | GAAGCAGAAATCAAAAAAGACAAGG | | TTCAATGGTTCAAGAAAAATA | | | |
| | | GAAAAAGTAGACAAAGCGATTTCTA | | TAAATGCACAAGTGCTTCCAA | | | |
| | | AATTTTCGATTGAAGGTAAAGTTATT | | ATGGTTATTGTAGATTGCCAGT | | | |
| | | AATTTTAATTCAATAGCAAAGGAAG | | AGTAGCAGGTGGTTGTCCACA | | | |
| | | CTAATGTTTCTAAATCATGGCTTTAT | | TGCGAATGCATGCTTAGATTG | | | |
| | | AAGGAACACGATATTAGGCAAAGAA | | CACTCACTTCTGTACCAGTAAG | | | |
| | | TCGAATCCCTTCGTGAGCGTCAAATA | | CAATTCTTACCACAGCACGAA | | | |
| | | ACAGCAAATGTAGTCTCAAAACCCA | | GAACAGTTAGAGCGTACAGAA | | | |
| | | AGAAAAGTTCTCGTTCGGAGGAAAT | | GAGTTATTAGCCATAGCTAAG | | | |
| | | CCTTATTAAAACCTTAAAAAG | | GATAAACAATGGCAAAGACAA | | | |
| | | | | GTAGAGACTAATAGCCGTGTT | | | |
| | | | | AAAGAGCGTTTAGAACAAATC | | | |
| | | | | ATTGGAAGTTTGACGGGGTAA | | | |
| 134 | WP_ | ATGAAAAGGATGAGTAGTTTAGCCT | 238 | ATGAAAAGGATGAGTAGTTTA | 269 | ACCCTAAGTACACGACTCAAC | 300 |
| | 056940799.1 | ACCACTTTGGCATTAAGCTGCGCTTT | | GCCTACCACTTTGGCATTAAGC | | GCTGTCATGCTTGTGGCACAA | |
| | | TATCCTAGCTCTAAGCAAAAGAAGCT | | TGCGCTTTTATCCTAGCTCTAA | | TCATGGGACATAATGGCTATA | |
| | | AATCAAGCTGAACTACGATGCTCAG | | GCAAAAGAAGCTAATCAAGCT | | AGAAATTAAAACTAAAAGAC | |
| | | CGTTTTGTTTATAATTCTTATGTAGG | | GAACTACGATGCTCAGCGTTTT | | CGTGAATGGACATGTCCAATT | |
| | | ACGCAATCGGAGCAGCTATCATGCC | | GTTTATAATTCTTATGTAGGAC | | TGTCATACTTACCATATTCGT | |
| | | AAACGCTATTTTCTCGTAAAACAAAG | | GCAATCGGAGCAGCTATCATG | | GATTGGAATGCGGCGGTAAA | |
| | | CAAGACAATGCCGTTTGCTTTTTCTA | | CCAAACGCTATTTTCTCGTAAA | | CATCCTTCAAAAAGGATTAGG | |
| | | TTCTCAATCGCTATGAAACAAAGCTG | | ACAAAGCAAGACAATGCCGTT | | CAGTTGGCAGAATCCTAAAA | |
| | | GCAGAAACAGTAGTCGCTAATAATG | | TGCTTTTTCTATTCTCAATCGC | | AACAAGCCACTTAAAAAGGT | |
| | | AGCTATTGGCTAAGCCTAAGAATATT | | TATGAAACAAAGCTGGCAGAA | | TGGAAGTCCGTTAGCAACTCG | |
| | | CGAGATGCTTATAGTTTCTTACGCAT | | ACAGTAGTCGCTAATAATGAG | | TGGACTAAAAAGGCTTTGGTA | |
| | | CAAAGAAATTGACAGTCTGGCGCTG | | CTATTGGCTAAGCCTAAGAAT | | ATTAGGCGACCTCTATCACTA | |
| | | GCCAATGCCATTCAGAACTATCATAA | | ATTCGAGATGCTTATAGTTTCT | | ATAGTCATAAGAATATTAGTG | |
| | | GGCTTGGAATAATTATCGCAAAATCG | | TACGCATCAAAGAAATTGACA | | ATAAGCCCAGCCCGTATCTAA | |
| | | GGCATGGAATCCCGACTTTCCACAAG | | GTCTGGCGCTGGCCAATGCCA | | GCAAGTTGGTGTTCTGGTTAG | |
| | | AAACGCAGCGATTGGACTTATCAAA | | TTCAGAACTATCATAAGGCTT | | CGCAAGCTAACGAGCCACCA | |
| | | CCAACTGCCAATACATTAAGCAGGA | | GGAATAATTATCGCAAAATCG | | AGCCCCAGAATTTATTCTGGG | |
| | | AGAAGCTTATTTGGACAACGGGACA | | GGCATGGAATCCCGACTTTCC | | GTAGTTGACTTGAACAATTTG | |
| | | GCTAAATTTATTGACGCTAAGCACGT | | ACAAGAAACGCAGCGATTGGA | | GTACAGGAATTCAAAGAATTC | |
| | | TAAATTGCCTAAGCTGGGTGTTGTCC | | CTTATCAAACCAACTGCCAAT | | GTGCAGCGTATGCTGAAAGTA | |
| | | GCATTGCTGGCTTGAGAAAGCTAATT | | ACATTAAGCAGGAAGAAGCTT | | AAATTCAGCCCCAGTTTTTAA | |
| | | AAAGAGCGTTTGTTTAAGCACATTCC | | ATTTGGACAACGGGACAGCTA | | TTTTTGAAAATTCAATCAAGA | |
| | | AACTAGAATCGGAACAGTGACGATT | | AATTTATTGACGCTAAGCACG | | TTGTTTTACCGGTAATTAAAA | |
| | | AAAAAGACCGCTGAAGATCAATTCT | | TTAAATTGCCTAAGCTGGGTGT | | A | |
| | | ATTTGTCAATGCAGTCAGGCAGCGAT | | TGTCCGCATTGCTGGCTTGAGA | | | |
| | | TTAGCTTTTGTTAAAGAGCTGCCCAA | | AAGCTAATTAAAGAGCGTTTG | | | |
| | | AACACAGAATCAAATCGGCATTGAT | | TTTAAGCACATTCCAACTAGA | | | |
| | | CTTAATTTAGACAACTTCTTGACCAA | | ATCGGAACAGTGACGATTAAA | | | |
| | | TTCTAATGGTGCCATGGTCGCTAATC | | AAGACCGCTGAAGATCAATTC | | | |
| | | CGCACTTCTACCGCCAAGCTGAAAA | | TATTTGTCAATGCAGTCAGGC | | | |
| | | GAAGCTCGCCCACGCCCAACGAGTA | | AGCGATTTAGCTTTTGTTAAAG | | | |
| | | CTCTCTCGCAGACAAAGACGTGCTAA | | AGCTGCCCAAAACACAGAATC | | | |
| | | GAAAGCAAGGCGCAATTTGCGGACG | | AAATCGGCATTGATCTTAATTT | | | |
| | | GCAAAGAACTATCAAAAGCAGCGCC | | AGACAACTTCTTGACCAATTCT | | | |
| | | TAATAGTCGCTAAACTGCACGACAG | | AATGGTGCCATGGTCGCTAAT | | | |
| | | GATTCGCAGACAGCGTGAAGACTTCT | | CCGCACTTCTACCGCCAAGCT | | | |
| | | TGCAGGTCCTCTCAACTGCATTAATC | | GAAAAGAAGCTCGCCCACGCC | | | |
| | | AAAAACCACGATTTAGTAGTTGCCG | | CAACGAGTACTCTCTCGCAGA | | | |
| | | AGGAGCTAAGAAGCAAGAACCTGTT | | CAAAGACGTGCTAAGAAAGCA | | | |
| | | AAAGAATCATGCCTTGTCCCAAGCG | | AGGCGCAATTTGCGGACGGCA | | | |
| | | ATCTCTGATGTTGGCTGGCGCAAGTT | | AAGAACTATCAAAAGCAGCGC | | | |
| | | TCTTAACATGCTTGCCTATAAAGGCA | | CTAATAGTCGCTAAACTGCAC | | | |
| | | GGCTATACGGTAAAGAGTTCATTACT | | GACAGGATTCGCAGACAGCGT | | | |
| | | ATTGACCCTAAGTACACGACTCAACG | | GAAGACTTCTTGCAGGTCCTCT | | | |
| | | CTGTCATGCTTGTGGCACAATCATGG | | CAACTGCATTAATCAAAAACC | | | |
| | | GACATAATGGCTATAAGAAATTAAA | | ACGATTTAGTAGTTGCCGAGG | | | |
| | | ACTAAAAGACCGTGAATGGACATGT | | AGCTAAGAAGCAAGAACCTGT | | | |
| | | CCAATTTGTCATACTTACCATATTCG | | TAAAGAATCATGCCTTGTCCC | | | |
| | | TGATTGGAATGCGGCGGTAAACATC | | AAGCGATCTCTGATGTTGGCT | | | |
| | | CTTCAAAAAGGATTAGGCAGTTGGC | | GGCGCAAGTTTCTTAACATGCT | | | |
| | | AGAATCCTAAAAAACAAGCCACTTA | | TGCCTATAAAGGCAGGCTATA | | | |
| | | AAAAGGTTGGAAGTCCGTTAGCAAC | | CGGTAAAGAGTTCATTACTATT | | | |
| | | TCGTGGACTAAAAAGGCTTTGGTAAT | | GACCCTAAGTACACGACTCAA | | | |
| | | TAGGCGACCTCTATCACTAATAGTCA | | CGCTGTCATGCTTGTGGCACA | | | |
| | | TAAGAATATTAGTGATAAGCCCAGC | | ATCATGGGACATAATGGCTAT | | | |
| | | CCGTATCTAAGCAAGTTGGTGTTCTG | | AAGAAATTAAAACTAAAAGAC | | | |
| | | GTTAGCGCAAGCTAACGAGCCACCA | | CGTGAATGGACATGTCCAATT | | | |
| | | AGCCCCAGAATTTATTCTGGGGTAGT | | TGTCATACTTACCATATTCGTG | | | |
| | | TGACTTGAACAATTTGGTACAGGAAT | | ATTGGAATGCGGCGGTAAACA | | | |
| | | TCAAAGAATTCGTGCAGCGTATGCTG | | TCCTTCAAAAAGGATTAGGCA | | | |
| | | AAAGTAAAATTCAGCCCCAGTTTTTA | | GTTGGCAGAATCCTAAAAAAC | | | |
| | | ATTTTTGAAAATTCAATCAAGATTGT | | AAGCCACTTAA | | | |
| | | TTTACCGGTAATTAAAAA | | | | | |
| 135 | WP_ | TTGCTTAAAGCAATAAAGTTTAGAAT | 239 | TTGCTTAAAGCAATAAAGTTT | 270 | TCAATATCAGAAACTATGCTT | 301 |
| | 073117560.1 | TTATCCCACCATAGAGCAAAAAACCT | | AGAATTTATCCCACCATAGAG | | TGGGAATGCTAGATGAGCGA | |
| | | TGATACACAAGCACTTTGGCTGTGCT | | CAAAAAACCTTGATACACAAG | | CACGCTATCAAGGTAGATAA | |
| | | AGGGTGGTCTATAACTACTTTTTAGC | | CACTTTGGCTGTGCTAGGGTG | | AACTAGGGTAGGGATTACCC | |
| | | ATACCGCCAAAAGCAATACGCACAA | | GTCTATAACTACTTTTTAGCAT | | GAAGTTACGCTTGTGGAGATT | |
| | | GGCATTAGAGAAAATTACTTTAGCAT | | ACCGCCAAAAGCAATACGCAC | | CCGCTAACGGGGCTGTAACCA | |
| | | GCAAAAGGCGCTCACTACTCTCAAA | | AAGGCATTAGAGAAAATTACT | | AGTATGGCTACATACTGGATA | |
| | | AAACAAGAGGCTTACGCTTACCTAA | | TTAGCATGCAAAAGGCGCTCA | | CTGCTAGTTATGGATCGTTGA | |
| | | GTGAATGCAACTCTCAAAGCTTGCAA | | CTACTCTCAAAAAACAAGAGG | | AGCAAGAAGCCCACCCGTCTT | |
| | | ATGGCACTAAGACAGCTGACAACAG | | CTTACGCTTACCTAAGTGAATG | | TAGCGGGTGGGTGATTCACTA | |
| | | CCTTTGATAGGTTTTTCTCTAAGCTG | | CAACTCTCAAAGCTTGCAAAT | | CCCTAGTTAATGATGTAAGAA | |
| | | GCAGATTATCCTAGATTCAAATCTAA | | GGCACTAAGACAGCTGACAAC | | ATATTTGGCAAGATTATGCCA | |
| | | AAAGCATTCTAAGCAGTCTTTCTGTG | | AGCCTTTGATAGGTTTTTCTCT | | ATAGCATTGGCAAAGATATA | |
| | | TCCCGCAACACTTAGAGATGGATTTA | | AAGCTGGCAGATTATCCTAGA | | AATACCGCCATTCAAGAGGCT | |
| | | GGCAACAACCAAGTCAAGCTACCCA | | TTCAAATCTAAAAAGCATTCT | | TTTACAAATCTAAGCAAAAAG | |
| | | AATTCAAAGAAGCTATTAAAGCCAA | | AAGCAGTCTTTCTGTGTCCCGC | | AAACAAGACTTCACTACATGG | |
| | | GTTTCATAGGCATTTGCCTACAAACT | | AACACTTAGAGATGGATTTAG | | CTTTTTGACTTCAAGGGAGAG | |
| | | CTATCGTCAAACAGGGGTTTATCTCT | | GCAACAACCAAGTCAAGCTAC | | AGTGATAAGAAGTTTCAATAC | |
| | | TGTGTAGCAGATAAATACTATCTCTC | | CCAAATTCAAAGAAGCTATTA | | ATTATGGATTTGGCTAACCGC | |
| | | TATAAGCTATGAGGATAATGAGCCT | | AAGCCAAGTTTCATAGGCATT | | GAGAAAGAAAGAATCTTAAG | |
| | | GAACCTAAACCCACAACTATCAGAA | | TGCCTACAAACTCTATCGTCAA | | AAATCTCAATTATGGGGAGG | |
| | | AAGCTGTGGGTTTAGATATGGGTTTA | | ACAGGGGTTTATCTCTTGTGTA | | AGGTGAACATGGATAATTACT | |
| | | GAGTCTTTAGTCATCGCCAGCAATGG | | GCAGATAAATACTATCTCTCTA | | CTGTGGTGTTTTCTTTGGTGTG | |
| | | CGTGTTCTACCCATATAAAAAGTTTT | | TAAGCTATGAGGATAATGAGC | | ATCTTTGTCTTACTCCTCTTAA | |
| | | TCCAGAATCTACAAACCAAACTCACT | | CTGAACCTAAACCCACAACTA | | | |
| | | AAAGCGCAAAGGAGATTGTCTAAGA | | TCAGAAAAGCTGTGGGTTTAG | | | |
| | | AACTAAAAGGTTCTAATAATAGAAA | | ATATGGGTTTAGAGTCTTTAGT | | | |
| | | AAAACAAGCTAAGAAAGTGGCACAA | | CATCGCCAGCAATGGCGTGTT | | | |
| | | ATCCACGCTTCTATCAGGAATAGCAG | | CTACCCATATAAAAAGTTTTTC | | | |
| | | AGAGGACTACCTACATAAAATCAGT | | CAGAATCTACAAACCAAACTC | | | |
| | | AATGAGATAACCAATCAATACGATTT | | ACTAAAGCGCAAAGGAGATTG | | | |
| | | GATAGCAGTAGAAACCTTGAAAGTT | | TCTAAGAAACTAAAAGGTTCT | | | |
| | | AGGAATTTGGTCAAAAACCACAAAC | | AATAATAGAAAAAAACAAGCT | | | |
| | | TCGCTAAGAGCATTGCCAATGCCAGC | | AAGAAAGTGGCACAAATCCAC | | | |
| | | TGGTCTAGGCTCATTAGTCTATTAGA | | GCTTCTATCAGGAATAGCAGA | | | |
| | | ATATAAGGCTGGTTGGAAGGGTAAA | | GAGGACTACCTACATAAAATC | | | |
| | | ACCCTTATCAAAATTGACCAATACTT | | AGTAATGAGATAACCAATCAA | | | |
| | | CCCTAGCTCTCAAATCTGCTCGACCT | | TACGATTTGATAGCAGTAGAA | | | |
| | | GTGGGAGCAACACAGGTAAAAAGCC | | ACCTTGAAAGTTAGGAATTTG | | | |
| | | ACTGCCTATTAGAAATTTTATCTACC | | GTCAAAAACCACAAACTCGCT | | | |
| | | CTTGTTGTCAAACATACCACCACAGA | | AAGAGCATTGCCAATGCCAGC | | | |
| | | GACCTAAACGCTAGCATCAATATCA | | TGGTCTAGGCTCATTAGTCTAT | | | |
| | | GAAACTATGCTTTGGGAATGCTAGAT | | TAGAATATAAGGCTGGTTGGA | | | |
| | | GAGCGACACGCTATCAAGGTAGATA | | AGGGTAAAACCCTTATCAAAA | | | |
| | | AAACTAGGGTAGGGATTACCCGAAG | | TTGACCAATACTTCCCTAGCTC | | | |
| | | TTACGCTTGTGGAGATTCCGCTAACG | | TCAAATCTGCTCGACCTGTGG | | | |
| | | GGGCTGTAACCAAGTATGGCTACAT | | GAGCAACACAGGTAAAAAGCC | | | |
| | | ACTGGATACTGCTAGTTATGGATCGT | | ACTGCCTATTAGAAATTTTATC | | | |
| | | TGAAGCAAGAAGCCCACCCGTCTTTA | | TACCCTTGTTGTCAAACATACC | | | |
| | | GCGGGTGGGTGATTCACTACCCTAGT | | ACCACAGAGACCTAAACGCTA | | | |
| | | TAATGATGTAAGAAATATTTGGCAA | | GCATCAATATCAGAAACTATG | | | |
| | | GATTATGCCAATAGCATTGGCAAAG | | CTTTGGGAATGCTAGATGAGC | | | |
| | | ATATAAATACCGCCATTCAAGAGGCT | | GACACGCTATCAAGGTAGATA | | | |
| | | TTTACAAATCTAAGCAAAAAGAAAC | | AAACTAGGGTAGGGATTACCC | | | |
| | | AAGACTTCACTACATGGCTTTTTGAC | | GAAGTTACGCTTGTGGAGATT | | | |
| | | TTCAAGGGAGAGAGTGATAAGAAGT | | CCGCTAACGGGGCTGTAACCA | | | |
| | | TTCAATACATTATGGATTTGGCTAAC | | AGTATGGCTACATACTGGATA | | | |
| | | CGCGAGAAAGAAAGAATCTTAAGAA | | CTGCTAGTTATGGATCGTTGAA | | | |
| | | ATCTCAATTATGGGGAGGAGGTGAA | | GCAAGAAGCCCACCCGTCTTT | | | |
| | | CATGGATAATTACTCTGTGGTGTTTT | | AGCGGGTGGGTGA | | | |
| | | CTTTGGTGTGATCTTTGTCTTACTCCT | | | | | |
| | | CTTAA | | | | | |

TABLE C
TnpB editor accessory proteins

| SEQ ID | Description | Sequence |
|---|---|---|
| 200 | Human adenosine | MAQTPAFDKPKVELHVHLDGSIKPETILYYGRRRGIALPANTAEG |
| | deaminase | LLNVIGMDKPLTLPDFLAKFDYYMPAIAGCREAIKRIAYEFVEMK |
| | | AKEGVVYVEVRYSPHLLANSKVEPIPWNQAEGDLTPDEVVALV |
| | | GQGLQEGERDFGVKARSILCCMRHQPNWSPKVVELCKKYQQQT |
| | | VVAIDLAGDETIPGSSLLPGHVQAYQEAVKSGIHRTVHAGEVGS |
| | | AEVVKEAVDILKTERLGHGYHTLEDQALYNRLRQENMHFEICP |
| | | WSSYLTGAWKPDTEHAVIRLKNDQANYSLNTDDPLIFKSTLDTD |
| | | YQMTKRDMGFTEEEFKRLNINAAKSSFLPEDEKRELLDLLYKAY |
| | | GMPPSASAGQNL |
| 201 | Human cytosine | MSNNALQTIINARLPGEEGLWQIHLQDGKISAIDAQSGVMPITEN |
| | deaminase | SLDAEQGLVIPPFVEPHIHLDTTQTAGQPNWNQSGTLFEGIERWA |
| | | ERKALLTHDDVKQRAWQTLKWQIANGIQHVRTHVDVSDATLTA |
| | | LKAMLEVKQEVAPWIDLQIVAFPQEGILSYPNGEALLEEALRLGA |
| | | DVVGAIPHFEFTREYGVESLHKTFALAQKYDRLIDVHCDEIDDEQ |
| | | SRFVETVAALAHHEGMGARVTASHTTAMHSYNGAYTSRLFRLL |
| | | KMSGINFVANPLVNIHLQGRFDTYPKRRGITRVKEMLESGINVCF |
| | | GHDDVFDPWYPLGTANMLQVLHMGLHVCQLMGYGQINDGLNL |
| | | ITHHSARTLNLQDYGIAAGNSANLIILPAENGFDALRRQVPVRYS |
| | | VRGGKVIASTQPAQTTVYLEQPEAIDYKR |
| 202 | Bacilus subtilis | MTQDELYMKEAIKEAKKAEEKGEVPIGAVLVINGEIIARAHNLRE |
| | tadA | TEQRSIAHAEMLVIDEACKALGTWRLEGATLYVTLEPCPMCAGA |
| | | VVLSRVEKVVFGAFDPKGGCSGTLMNLLQEERFNHQAEVVSGV |
| | | LEEECGGMLSAFFRELRKKKKAARKNLSE |
| 203 | E Coli tadA | MRRAFITGVFFLSEVEFSHEYWMRHALTLAKRAWDEREVPVGA |
| | | VLVHNNRVIGEGWNRPIGRHDPTAHAEIMALRQGGLVMQNYRL |
| | | IDATLYVTLEPCVMCAGAMIHSRIGRVVFGARDAKTGAAGSLM |
| | | DVLHHPGMNHRVEITEGILADECAALLSDFFRMRRQEIKAQKKA |
| | | QSSTD |
| 204 | S cerevisiae tadA | MQHIKHMRTAVRLARYALDHDETPVACIFVHTPTGQVMAYGM |
| | | NDTNKSLTGVAHAEFMGIDQIKAMLGSRGVVDVFKDITLYVTVE |
| | | PCIMCASALKQLDIGKVVFGCGNERFGGNGTVLSVNHDTCTLVP |
| | | KNNSAAGYESIPGILRKEAIMLLRYFYVRQNERAPKPRSKSDRVL |
| | | DKNTFPP |
| 205 | Staphylococcus | MKKNHDYMRLAINEAHKAKALGEVPIGAVIVKEGQVIARAHNL |
| | epidermidis tadA | RETLQQPTAHAEHIAIERASEVVGSWRLEECTLYVTLEPCVMCA |
| | | GTIIMSRIPKVVYGATDPKGGCSGSLMNLLEQPQFNHRTTVEKGI |
| | | LEEECAELLRSFFKEIREKKKAEKQGKIQKDINLLK |
| 206 | M-MLV GalPol | MGQTVTTPLSLTLQHWGDVQRIASNQSVDVKKRRWVTFCSAEW |
| | | PTFNVGWPQDGTFNLGVISQVKSRVFCPGPHGHPDQVPYIVTWE |
| | | ALAYDPPPWVKPFVSPKPPPLPTAPVLPPGPSAQPPSRSALYPALT |
| | | LSIKSKPPKPQVLPDSGGPLIDLLTEDPPPYGVQPSSSARENNEEE |
| | | AATTSEVSPPSPMVSRLRGRRDPPAADSTTSQAFPLRMGGDGQL |
| | | QYWPFSSSDLYNWKNNNPSFSEDPGKLTALIESVLITHQPTWDD |
| | | CQQLLGTLLTGEEKQRVLLEAGKAVRGNDGRPTQLPNEVNAAF |
| | | PLERPDWDYTTTEGRNHLVLYRQLLLAGLQNAGRSPTNLAKVK |
| | | GITQGPNESPSAFLERLKEAYRRYTPYDPEDPGQETNVSMSFIWQ |
| | | SAPDIGRKLERLEDLKSKTLGDLVREAEKIFNKRETPEEREERIRR |
| | | EIEEKEERRRAEDEQRERERDRRRHREMSKLLATVVIGQRQDRQ |
| | | GGERRRPQLDKDQCAYCKEKGHWAKDCPKKPRGPRGPRPQTSL |
| | | LTLGDXGGQGQEPPPEPRITLKVGGQPVTFLVDTGAQHSVLTQN |
| | | PGPLSDKSAWVQGATGGKRYRWTTDRKVHLATGKVTHSFLHVP |
| | | DCPYPLLGRDLLTKLKAQIHFEGSGAQVVGPMGQPLQVLTLNIE |
| | | NKYRLHETSKEPDVPLGSTWLSDFPQAWAETGGMGLAVRQAPL |
| | | IIPLKATSTPVSIKQYPMSQEARLGIKPHIQRLLDQGILVPCQSPWN |
| | | TPLLPVKKPGTNDYRPVQDLREVNKRVEDIHPTVPNPYNLLSGLP |
| | | PSHQWYTVLDLKDAFFCLRLHPTSQPLFAFEWRDPEMGISGQLT |
| | | WTRLPQGFKNSPTLFDEALHRDLADFRIQHPDLILLQYVDDLLLA |
| | | ATSEQDCQRGTRALLQTLGNLGYRASAKKAQICQKQVKYLGYL |
| | | LKEGQRWLTEARKETVMGQPTPKTPRQLREFLGTAGFCRLWIPG |
| | | FAEMAAPLYPLTKTGTLFNWGPDQQKAYQEIKQALLTAPALGLP |
| | | DLTKPFELFVDEKQGYAKGVLTQKLGPWRRPVAYLSKKLDPVA |
| | | AGWPPCLRMVAAIAVLTKDAGKLTMGQPLVILAPHAVEALVKQ |
| | | PPDRWLSNARMTHYQAMLLDTDRVQFGPVVALNPATLLPLPEK |
| | | EAPHDCLEILAETHGTRPDLTDQPIPDADYTWYTDGSSFLQEGQR |
| | | RAGAAVTTETEVIWARALPAGTSAQRAELIALTQALKMAEGKK |
| | | LNVYTDSRYAFATAHVHGEIYRRRGLLTSEGREIKNKNEILALLK |
| | | ALFLPKRLSIIHCPGHQKGNSAEARGNRMADQAAREAAMKAVL |
| | | ETSTLLIEDSTPYTPPHFHYTETDLKRLRELGATYNQTKGYWVLQ |
| | | GKPVMPDQSVFELLDSLHRLTHPSPQKMKALLDREESPYYMLNR |
| | | DRTIQYVTETCTACAQVNASKAKIGAGVRVRGHRPGTHWEVDF |
| | | TEVKPGLYGYKYLLVFVDTFSGWVEAFPTKRETAKVVTKKLLE |
| | | DIFPRFGMPQVLGSDNGPAFASQVSQSVADLLGIDWKLHCAYRP |
| | | QSSGQVERMNRTIKETLTKLTLASGTRDWVLLLPLALYRARNTP |
| | | GPHGLTPYEILYGAPPPLVNFHDPEMSKLTNSPSLQAHLQALQAV |
| | | QQEVWKPLAAAYQDQLDQPVIPHPFRVGDAVWVRRHQTKNLEP |
| | | RWKGPYTVLLTTPTALKVDGISAWIHAAHVKAATTPPAGTAWK |
| | | VQRSQNPLKIRLTRGAP |
| 207 | M-MLV reverse | QAWAETGGMGLAVRQAPLIIPLKATSTPVSIKQYPMSQEARLGIK |
| | transcriptase | PHIQRLLDQGILVPCQSPWNTPLLPVKKPGTNDYRPVQDLREVN |
| | | KRVEDIHPTVPNPYNLLSGLPPSHQWYTVLDLKDAFFCLRLHPTS |
| | | QPLFAFEWRDPEMGISGQLTWTRLPQGFKNSPTLFDEALHRDLA |
| | | DFRIQHPDLILLQYVDDLLLAATSEQDCQRGTRALLQTLGNLGY |
| | | RASAKKAQICQKQVKYLGYLLKEGQRWLTEARKETVMGQPTPK |
| | | TPRQLREFLGTAGFCRLWIPGFAEMAAPLYPLTKTGTLFNWGPD |
| | | QQKAYQEIKQALLTAPALGLPDLTKPFELFVDEKQGYAKGVLTQ |
| | | KLGPWRRPVAYLSKKLDPVAAGWPPCLRMVAAIAVLTKDAGKL |
| | | TMGQPLVILAPHAVEALVKQPPDRWLSNARMTHYQAMLLDTDR |
| | | VQFGPVVALNPATLLPLPEKEAPHDCLEILAETHGTRPDLTDQPIP |
| | | DADYTWYTDGSSFLQEGQRRAGAAVTTETEVIWARALPAGTSA |
| | | QRAELIALTQALKMAEGKKLNVYTDSRYAFATAHVHGEIYRRR |
| | | GLLTSEGREIKNKNEILALLKALFLPKRLSIIHCPGHQKGNSAEAR |
| | | GNRMADQAAREAAMKAVLETSTLL |
| 208 | Murine reverse | HLATGKVTHSFLHVPDCPYPLLGRDLLTKLKAQIHFEGSGAQVV |
| | transcriptase | GPKGQPLQVLTLNLEDEYRLHETSAEPEASPGSTWLSDFPQAWA |
| | | ETGGMGLAVRQAPLIIPLKATSTPVSIKQYPMSQEARLGIKPHIQR |
| | | LLDQGILV |

TABLE D
Linker sequences

| SEQ | | |
|---|---|---|
| ID | Description | Sequence |
| 136 | GlySer linker | GGS |
| | based on GGS | |
| | repeating unit | |
| 137 | GlySer linker | GGS GGS |
| | based on GGS | |
| | repeating unit | |
| 138 | GlySer linker | GGS GGS GGS |
| | based on GGS | |
| | repeating unit | |
| 139 | GlySer linker | GGS GGS GGS GGS |
| | based on GGS | |
| | repeating unit | |
| 140 | GlySer linker | GGS GGS GGS GGS GGS |
| | based on GGS | |
| | repeating unit | |
| 141 | GlySer linker | GGS GGS GGS GGS GGS GGS |
| | based on GGS | |
| | repeating unit | |
| 142 | GlySer linker | GGS GGS GGS GGS GGS GGS GGS |
| | based on GGS | |
| | repeating unit | |
| 143 | GlySer linker | GGS GGS GGS GGS GGS GGS GGS GGS |
| | based on GGS | |
| | repeating unit | |
| 144 | GlySer linker | GGS GGS GGS GGS GGS GGS GGS GGS GGS |
| | based on GGS | |
| | repeating unit | |
| 145 | GlySer linker | GGS GGS GGS GGS GGS GGS GGS GGS GGS GGS |
| | based on GGS | |
| | repeating unit | |
| 146 | GlySer linker | GGS GGS GGS GGS GGS GGS GGS GGS GGS GGS GGS |
| | based on GGS | |
| | repeating unit | |
| 147 | GlySer linker | GGS GGS GGS GGS GGS GGS GGS GGS GGS GGS GGS GGS |
| | based on GGS | |
| | repeating unit | |
| 148 | GlySer linker | GGS GGS GGS GGS GGS GGS GGS GGS GGS GGS GGS GGS GGS |
| | based on GGS | |
| | repeating unit | |
| 149 | GlySer linker | GGS GGS GGS GGS GGS GGS GGS GGS GGS GGS GGS GGS GGS GGS |
| | based on GGS | |
| | repeating unit | |
| 150 | GlySer linker | GGS GGS GGS GGS GGS GGS GGS GGS GGS GGS GGS GGS GGS GGS |
| | based on GGS | GGS |
| | repeating unit | |
| 151 | GlySer linker | GSG |
| | based on GSG | |
| | repeating unit | |
| 152 | GlySer linker | GSG GSG |
| | based on GSG | |
| | repeating unit | |
| 153 | GlySer linker | GSG GSG GSG |
| | based on GSG | |
| | repeating unit | |
| 154 | GlySer linker | GSG GSG GSG GSG |
| | based on GSG | |
| | repeating unit | |
| 155 | GlySer linker | GSG GSG GSG GSG GSG |
| | based on GSG | |
| | repeating unit | |
| 156 | GlySer linker | GSG GSG GSG GSG GSG GSG |
| | based on GSG | |
| | repeating unit | |
| 157 | GlySer linker | GSG GSG GSG GSG GSG GSG GSG GSG |
| | based on GSG | |
| | repeating unit | |
| 158 | GlySer linker | GSG GSG GSG GSG GSG GSG GSG GSG GSG |
| | based on GSG | |
| | repeating unit | |
| 159 | GlySer linker | GSG GSG GSG GSG GSG GSG GSG GSG GSG GSG |
| | based on GSG | |
| | repeating unit | |
| 160 | GlySer linker | GSG GSG GSG GSG GSG GSG GSG GSG GSG GSG GSG |
| | based on GSG | |
| | repeating unit | |
| 161 | GlySer linker | GSG GSG GSG GSG GSG GSG GSG GSG GSG GSG GSG GSG |
| | based on GSG | |
| | repeating unit | |
| 162 | GlySer linker | GSG GSG GSG GSG GSG GSG GSG GSG GSG GSG GSG GSG GSG |
| | based on GSG | |
| | repeating unit | |
| 163 | GlySer linker | GSG GSG GSG GSG GSG GSG GSG GSG GSG GSG GSG GSG GSG GSG |
| | based on GSG | |
| | repeating unit | |
| 164 | GlySer linker | GSG GSG GSG GSG GSG GSG GSG GSG GSG GSG GSG GSG GSG GSG |
| | based on GSG | GSG |
| | repeating unit | |
| 165 | GlySer linker | GGGS |
| | based on GGGS | |
| | repeating unit | |
| 166 | GlySer linker | GGGS GGGS |
| | based on GGGS | |
| | repeating unit | |
| 167 | GlySer linker | GGGS GGGS GGGS |
| | based on GGGS | |
| | repeating unit | |
| 168 | GlySer linker | GGGS GGGS GGGS GGGS |
| | based on GGGS | |
| | repeating unit | |
| 169 | GlySer linker | GGGS GGGS GGGS GGGS GGGS |
| | based on GGGS | |
| | repeating unit | |
| 170 | GlySer linker | GGGS GGGS GGGS GGGS GGGS GGGS |
| | based on GGGS | |
| | repeating unit | |
| 171 | GlySer linker | GGGS GGGS GGGS GGGS GGGS GGGS GGGS |
| | based on GGGS | |
| | repeating unit | |
| 172 | GlySer linker | GGGS GGGS GGGS GGGS GGGS GGGS GGGS GGGS |
| | based on GGGS | |
| | repeating unit | |
| 173 | GlySer linker | GGGS GGGS GGGS GGGS GGGS GGGS GGGS GGGS GGGS |
| | based on GGGS | |
| | repeating unit | |
| 174 | GlySer linker | GGGS GGGS GGGS GGGS GGGS GGGS GGGS GGGS GGGS GGGS |
| | based on GGGS | |
| | repeating unit | |
| 175 | GlySer linker | GGGS GGGS GGGS GGGS GGGS GGGS GGGS GGGS GGGS GGGS |
| | based on GGGS | GGGS |
| | repeating unit | |
| 176 | GlySer linker | GGGS GGGS GGGS GGGS GGGS GGGS GGGS GGGS GGGS GGGS |
| | based on GGGS | GGGS GGGS |
| | repeating unit | |
| 177 | GlySer linker | GGGS GGGS GGGS GGGS GGGS GGGS GGGS GGGS GGGS GGGS |
| | based on GGGS | GGGS GGGS GGGS |
| | repeating unit | |
| 178 | GlySer linker | GGGS GGGS GGGS GGGS GGGS GGGS GGGS GGGS GGGS GGGS |
| | based on GGGS | GGGS GGGS GGGS GGGS |
| | repeating unit | |
| 179 | GlySer linker | GGGS GGGS GGGS GGGS GGGS GGGS GGGS GGGS GGGS GGGS |
| | based on GGGS | GGGS GGGS GGGS GGGS GGGS |
| | repeating unit | |
| 180 | GlySer linker | GGGGS |
| | based on | |
| | GGGGS | |
| | repeating unit | |
| 181 | GlySer linker | GGGGS GGGGS |
| | based on | |
| | GGGGS | |
| | repeating unit | |
| 182 | GlySer linker | GGGGS GGGGS GGGGS |
| | based on | |
| | GGGGS | |
| | repeating unit | |
| 183 | GlySer linker | GGGGS GGGGS GGGGS GGGGS |
| | based on | |
| | GGGGS | |
| | repeating unit | |
| 184 | GlySer linker | GGGGS GGGGS GGGGS GGGGS GGGGS |
| | based on | |
| | GGGGS | |
| | repeating unit | |
| 185 | GlySer linker | GGGGS GGGGS GGGGS GGGGS GGGGS GGGGS |
| | based on | |
| | GGGGS | |
| | repeating unit | |
| 186 | GlySer linker | GGGGS GGGGS GGGGS GGGGS GGGGS GGGGS GGGGS |
| | based on | |
| | GGGGS | |
| | repeating unit | |
| 187 | GlySer linker | GGGGS GGGGS GGGGS GGGGS GGGGS GGGGS GGGGS GGGGS |
| | based on | |
| | GGGGS | |
| | repeating unit | |
| 188 | GlySer linker | GGGGS GGGGS GGGGS GGGGS GGGGS GGGGS GGGGS GGGGS |
| | based on | GGGGS |
| | GGGGS | |
| | repeating unit | |
| 189 | GlySer linker | GGGGS GGGGS GGGGS GGGGS GGGGS GGGGS GGGGS GGGGS |
| | based on | GGGGS GGGGS |
| | GGGGS | |
| | repeating unit | |
| 190 | GlySer linker | GGGGS GGGGS GGGGS GGGGS GGGGS GGGGS GGGGS GGGGS |
| | based on | GGGGS GGGGS GGGGS |
| | GGGGS | |
| | repeating unit | |
| 191 | GlySer linker | GGGGS GGGGS GGGGS GGGGS GGGGS GGGGS GGGGS GGGGS |
| | based on | GGGGS GGGGS GGGGS GGGGS |
| | GGGGS | |
| | repeating unit | |
| 192 | GlySer linker | GGGGS GGGGS GGGGS GGGGS GGGGS GGGGS GGGGS GGGGS |
| | based on | GGGGS GGGGS GGGGS GGGGS GGGGS |
| | GGGGS | |
| | repeating unit | |
| 193 | GlySer linker | GGGGS GGGGS GGGGS GGGGS GGGGS GGGGS GGGGS GGGGS |
| | based on | GGGGS GGGGS GGGGS GGGGS GGGGS GGGGS |
| | GGGGS | |
| | repeating unit | |
| 194 | GlySer linker | GGGGS GGGGS GGGGS GGGGS GGGGS GGGGS GGGGS GGGGS |
| | based on | GGGGS GGGGS GGGGS GGGGS GGGGS GGGGS GGGGS |
| | GGGGS | |
| | repeating unit | |
| 195 | linker | LEPGEKPYKCPECGKSFSQSGALTRHQRTHTR |
| 196 | XTEN linker | TCGGGATCTGAGACGCCTGGGACCTCGGAATCGGCTACGCCCGAAA |
| | | GT |
| 197 | linker | LEPGEKPYKCPECGKSFSQSGALTRHQRTHTRPECGKSFSQSGALTRHQ |
| | | RTHTR |
| 198 | linker | LEPGEKPYKCPECGKSFSQSGALTRHQRTHTRLEPGEKPYKCPECGKSF |
| | | SQSGALTRHQRTHTRPECGKSFSQSGALTRHQRTHTR |
| 199 | linker | PKKKRKVEASSPKKRKVEAS |

### H. Incorporation by Reference

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control.

### I. EXAMPLES

The following are examples of methods and compositions of the present disclosure. It is understood that various other embodiments may be practiced, given the general description provided herein.

### 1. Example 1: Production of Nanoparticle Compositions

A nanoparticle composition may be produced as described in US patent application US20170210697A1, which is incorporated herein by reference in its entirety.

In order to investigate safe and efficacious nanoparticle compositions for use in the delivery of TnpB editing systems, a range of formulations are prepared and tested. Specifically, the particular elements and ratios thereof in the lipid component of nanoparticle compositions are optimized.

Nanoparticles can be made with mixing processes such as microfluidics and T-junction mixing of two fluid streams, one of which contains the editing system components and the other has the lipid components.

Lipid compositions are prepared by combining an ionizable lipid, a phospholipid (such as DOPE or DSPC, obtainable from Avanti Polar Lipids, Alabaster, Ala.), a PEG lipid (such as PEG-DMG, obtainable from Avanti Polar Lipids, Alabaster, Ala.), and a structural lipid (such as cholesterol, obtainable from Sigma-Aldrich, Taufkirchen, Germany, or a cholesterol analog) in ethanol. Lipids are combined to yield desired molar ratios and diluted with water and ethanol.

Nanoparticle compositions may be prepared by combining a lipid solution with a solution including the editing system components. The lipid solution is rapidly injected using, for example, a NanoAssemblr® microfluidic based system, into the editing system components solution.

Solutions of the editing system components in deionized water may be diluted in citrate buffer to form a stock solution.

Nanoparticle compositions can be processed by dialysis to remove ethanol and achieve buffer exchange. Formulations are dialyzed against a buffer such as phosphate buffered saline (PBS), Tris-HCl, or sodium citrate, using, for example, Slide-A-Lyzer cassettes (Thermo Fisher Scientific Inc., Rockford, Ill.). The resulting nanoparticle suspension is filtered through sterile filters (Sarstedt, Nümbrecht, Germany) into glass vials and sealed with crimp closures. Alternatively, a Tangential Flow Filtration (TFF) system, such as a Spectrum KrosFlo system, may be used.

The method described above induces nano-precipitation and particle formation. Alternative processes including, but not limited to, T-junction and direct injection, may be used to achieve the same nano-precipitation.

### 2. Example 1a: Exemplary Nanoparticle Formulation Procedure

Ionizable lipids, phospholipids, structural lipids (eg. Cholesterol or other sterols), PEGlipids and megalin ligand modified PEG lipids are dissolved in ethanol. The ionizable lipids mol % can be from 30-70%, phospholipids mol % can be 5-20%, sterols mol % can be 20-60%, PEG lipid mol % can be 0.1-10%, and megalin ligand modified PEG lipid mol % can be 0.01-10%. The lipid solution is mixed with an acidic buffer containing mRNA on a mixing device, such as a NanoAssemblr® microfluidic systems, to form LNPs. To adjust LNP particle size, the volume ratio of lipid solution to mRNA solution can be varied from 1:1 to 20:1, mRNA concentration in aqueous buffer can be 0.01 mg/mL to 10 mg/mL, N/P ratio can be 1 to 50 and different identities of PEG lipids or other polymers can be used. After the LNP is formed from the mixing device, aqueous buffer is added to reduce the ethanol concentration. The volume of aqueous buffer can be 0.1 to 100 volume of LNP volume coming out of the mixing device. The LNPs are further dialyzed against aqueous and concentrated to a desired concentration. The particle size of LNPs is measured by dynamic light scattering (DLS), for example, by using a Zetasizer Ultra (Malvern Panalytical). RNA encapsulation efficiency is determined, for example, by Quant-it™ RiboGreen assay.

### 3. Example 2: Characterization of Nanoparticle Compositions

A nanoparticle composition may be characterized as described in US patent application US20170210697A1, which is incorporated herein by reference in its entirety.

Particle size, polydispersity index (PDI), and the zeta potential of a nanoparticle composition can be determined using, for example, a Zetasizer Nano ZS (Malvern Instruments Ltd, Malvern, Worcestershire, UK), or a Wyatt DynaPro plate reader.

Ultraviolet-visible spectroscopy can be used to determine the concentration of editing system components in nanoparticle compositions. The formulation may be diluted in PBS then added to a mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, Calif.). The concentration of editing system components in the nanoparticle composition can be calculated based on the extinction coefficient of the editing system components used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, Calif.) can be used to evaluate the encapsulation of an RNA by the nanoparticle composition. The samples are diluted in a TE buffer solution. Portions of the diluted samples are transferred to a polystyrene 96 well plate and either TE buffer or a 2% Triton X-100 solution is added to the wells. The plate is incubated at, for example, a temperature of 37° C. for 15 minutes. The RIBOGREEN® reagent is diluted in TE buffer, and this solution is added to each well. The fluorescence intensity can be measured using a fluorescence plate reader (Wallac Victor 1420 Multilablel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### 4. Example 3: In Vivo Studies Including Protein Expression by Organ

Delivery to a target organ may be assessed as described in US patent application US20170210697A1, which is incorporated herein by reference in its entirety.

In order to monitor how effectively various nanoparticle compositions deliver polynucleotides to targeted cells, different nanoparticle compositions including a particular polynucleotide are prepared and administered to rodent populations. Mice are intravenously, intramuscularly, intraarterially, or intratumorally administered a single dose of a nanoparticle composition. In some instances, mice may be made to inhale doses. Dose sizes may range from 0.001 mg/kg to 10 mg/kg, where 10 mg/kg describes a dose including 10 mg of polynucleotide in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. Time courses of protein expression can also be evaluated. Samples collected from the rodents for evaluation may include blood, sera, and tissue (for example, muscle tissue from the site of an intramuscular injection and internal tissue); sample collection may involve sacrifice of the animals.

For example, LNP formulations including RNA encoding a detectable protein such as luciferase may be administered intravenously to mice at a dosage of, for example, 0.5 mg/kg. A standard MC3 formulation and a PBS control may also be tested. Bioluminescence in various organs, such as the liver, lung, spleen, and femur, may be measured after 6 hours.

Nanoparticle compositions including protein coding RNA are useful in the evaluation of the efficacy and usefulness of various formulations for the delivery of polynucleotides. Higher levels of protein expression induced by administration of a composition including protein coding RNA will be indicative of higher RNA translation and/or nanoparticle composition RNA delivery efficiencies. As the non-RNA components are not thought to affect translational machineries themselves, a higher level of protein expression is likely indicative of a higher efficiency of delivery of the RNA by a given nanoparticle composition relative to other nanoparticle compositions or the absence thereof.

### 5. Example 4: Toxicity, Cytokine Induction, and Complement Activation

Toxicity of the LNP compositions of the disclosure may be analyzed as described by international patent application WO2016118724 and/or US20170210697A1, which are incorporated herein by reference in its entirety.

### 6. Example 4a: Liver Toxicity

RNA encoding a detectable protein is generated and loaded into lipid nanoparticles. The nanoparticles are administered to mice, and expression of the detectable protein as well as levels of certain liver enzymes are measured. Additional mice may be dosed with a reference LNP formulation, such as one containing MC3, as a comparison. To assess dose response, mice may be given varying levels of the LNP formulations. Liver enzymes, such as alanine transaminase (ALT) and aspartate transaminase (AST), may be measured to assess liver toxicity. In some embodiments, creatine phosphokinase (CPK) may also be measured to assess cardiac or muscular toxicity. In some embodiments, a pharmaceutical composition described herein provides a safer toxicity profile than a reference pharmaceutical composition, such as one containing MC3.

### 7. Example 4b: Cytokine Induction

The introduction of foreign material into a mammalian body induces an innate immune response that promotes cytokine production. Such immune responses to, for example, nanoparticle compositions including editing systems of the disclosure, are undesirable. The induction of certain cytokines is thus measured to evaluate the efficacy of nanoparticle compositions and the inflammatory response. The concentrations of various cytokines in mice upon intravenous administration of nanoparticle compositions at a dosage of 0.5 mg/kg are measured at 6 hours. The standard MC3 formulation and a PBS control may also be tested. Cytokines including TNF-α, IFN-γ, IP-10, MCP-1, IFN-a, IL-6, and IL-5 may be measured. In some embodiments, IP-10 and IL-6 are measured. In some embodiments, histamine levels may also be measured. In some embodiments, a pharmaceutical composition described herein provides an improved inflammatory profile than a reference pharmaceutical composition, such as one containing MC3.

### 8. Example 4c: Complement Activation

Complement activation assists in the clearance of pathogens from an organism. As it is undesirable that a subject's body recognize a nanoparticle composition as a foreign invader, low complement system activation upon administration of such a composition is preferred. The complex sC5b-9 is a marker for the activation of the complement system. Thus, human cells are contacted in vitro with nanoparticle compositions and are evaluated for sC5b-9 levels.

### 9. Example 5: LNP Optimization

LNP compositions may be optimized as described by US patent application US20170210697A1, which is incorporated herein by reference in its entirety.

### 10. Example 5a: Optimization of Ionizable Lipid

As smaller particles with higher encapsulation efficiencies are generally desirable, the relative amounts of various elements in lipid components of nanoparticle compositions are optimized according to these parameters.

An ionizable lipid is selected for optimization. The relative amount of the ionizable lipid is varied between 30 mol % and 60 mol % in compositions that can include DOPE or DSPC as phospholipids to determine the optimal amount of the ionizable lipid in the formulations. Formulations are prepared using a standardized process with a water to ethanol ratio in the lipid-mRNA solution of, for example, 3:1 and a rate of injection of the lipid solution into the mRNA solution of, for example, 12 mL/min on a NanoAssemblr® microfluidic based system. These parameters may be altered depending on, for example, the lipids used and the target particle size. This method induces nano-precipitation and particle formation. Alternative processes including, but not limited to, T-junction or direct injection, may also be used to achieve the same nano-precipitation.

Formulations producing the smallest particles with the highest encapsulation efficiencies are generally preferred. Compositions are also evaluated for their detectable protein expression levels and cytokine profiles.

### 11. Example 5b: Optimization of Phospholipid

The relative amount of phospholipid in a lipid component of a nanoparticle composition is varied to further optimize the formulation. An ionizable lipid is selected for use in the nanoparticle composition and a phospholipid such as DOPE and DSPC are selected. Additional phospholipids can also be evaluated. Nanoparticle compositions are prepared with the relative phospholipid content varying between 0 mol % and 30 mol %. Compositions are evaluated for their size, encapsulation efficiency, detectable protein expression levels, and cytokine profiles.

### 12. Example 5c: Optimization of Structural Lipid

The relative amount of structural lipid in a lipid component of a nanoparticle composition is varied to further optimize the formulation. An ionizable lipid is selected for use in the nanoparticle composition and cholesterol or a cholesterol analog is selected as a structural lipid. Additional structural lipids can also be evaluated. Nanoparticle compositions are prepared with the relative structural lipid content varying between 18.5 mol % and 48.5 mol %. Compositions are evaluated for their size, encapsulation efficiency, detectable protein expression levels, and cytokine profiles.

### 13. Example 5d: Optimization of PEG Lipid

The relative amount of PEG lipid in a lipid component of a nanoparticle composition is varied to further optimize the formulation. An ionizable lipid is selected for use in the nanoparticle composition and a PEG lipid such as PEG-DMG or PEG-DSPE is selected. Additional PEG lipids can also be evaluated. Nanoparticle compositions are prepared with the relative PEG lipid content varying between 0 mol % and 10 mol %. Compositions are evaluated for their size, encapsulation efficiency, detectable protein expression levels, and cytokine profiles. For formulations wherein the PEG lipid is conjugated to one or more targeting moieties, the ratio of conjugated and non-conjugated PEG lipid can also be optimized in order to increase or decrease the relative amount of targeting moiety present on the outer surface of the nanoparticles.

### 14. Example 5e: Optimization of Particle Sizes

The fenestration sizes for different bodily organs often vary; for example, the kidney is known to have a smaller fenestration size than the liver. Thus, targeting delivery of an editing system (e.g., specifically delivering) to a particular organ or group of organs may require the administration of nanoparticle compositions with different particle sizes. In order to investigate this effect, nanoparticle compositions are prepared with a variety of particle sizes using a Nanoassemblr® instrument. Nanoparticle compositions include an RNA encoding Luc. Each differently sized nanoparticle composition is subsequently administered to mice to evaluate the effect of particle size on delivery selectivity. Luc expression in two or more organs or groups of organs can be measured using bioluminescence to evaluate the relative expression in each organ.

A number of parameters can be adjusted in order to optimize the particle size of the nanoparticles. Exemplary parameters include, but are not limited to, the identity of the PEG lipid, mol % of the PEG lipid in the LNP formulation, the identity of the structural lipid, mol % of the structural lipid in the LNP formulation, the identity of the phospholipid, mol % of the phospholipid in the LNP formulation, the identity of the ionizable lipid, mol % of the ionizable lipid in the LNP formulation, identity of lipid components covalently bound to one or more targeting moieties, mol % of said targeting moiety bound lipids in the LNP formulation, flow rate of the Nanoassemblr® instrument in the preparation of the formulation, concentration of the mixing solutions used in the formulation, buffers used in the preparation of the formulation, and duration of formulation mixing.

### 15. Example 6: Construction and Testing a TnpB-TnpB Fusion

To generate a TnpB-TnpB fusion construct, a human codon optimized TnpB ORF is amplified from plasmids with primer overhangs containing the desired linker sequence. In addition, the desired nuclear localization signal is encoded on the amplification primers. Two amplified fragments containing the TnpB ORFs are stitched together using Gibson Assembly Master Mix (NEB), into the pCDNA3.1 vector (Thermo Scientific). The cognate reRNA for the indicated TnpB ortholog is cloned under the control of the U6 promoter using Gibson Assembly Master Mix (NEB), into the pZ147-BvCas12b-sgRNA-scaffold vector (Addgene). The reRNA may be designed such that the guide sequence is replaced to match the desired target sequence with a cognate TAM for the indicated TnpB ortholog, particularly the human genome in the case that the construct is to be used in a human genome editing experiment. In experiments where one of the TnpB monomers is mutated, the desired mutation is created by amplifying the entire plasmid in a single amplicon where the primer encodes the mutation and is cloned using the KLD enzyme mix (NEB). To evaluate the efficacy of the system, 100 ng of each of the TnpB-TnpB plasmid and the reRNA plasmid are transfected using Lipo 3000 (Thermo Scientific) into HEK293FT cells into a single well of a 96-well plate. The cells are left to incubate for 72 hours before they are harvested for sequencing. Quick Extract DNA solution is to extract gDNA from cells for subsequent NGS analysis of the targeted loci.

### 16. Example 7: Constructing and Testing a TnpB-Deaminase Fusion

To generate a TnpB deaminase fusion construct, both a human codon optimized TnpB ORF and a desired human codon optimized deaminase are amplified from plasmids with primer overhangs containing the desired linker sequence. In addition, the desired nuclear localization signal is encoded on the amplification primers. Two amplified fragments containing the TnpB ORF and deaminase are stitched together using Gibson Assembly Master Mix (NEB), into the pCDNA3.1 vector (Thermo Scientific). The cognate reRNA for the indicated TnpB ortholog is cloned under the control of the U6 promoter using Gibson Assembly Master Mix (NEB), into the pZ147-BvCas12b-sgRNA-scaffold vector (Addgene). The reRNA may be designed such that the guide sequence is replaced to match the desired target sequence with a cognate TAM for the indicated TnpB ortholog, particularly the human genome in the case that the construct is to be used in a human genome editing experiment. In experiments where the TnpB is mutated, the desired mutation is created by amplifying the entire plasmid in a single amplicon where the primer encodes the mutation and is cloned using the KLD enzyme mix (NEB). In experiments where a second TnpB ORF is included in the system, it is either cloned into the same construct, as described in Example 1, or expressed from a separate pCDNA3.1 vector. To evaluate the efficacy of the system, 100 ng of each of the TnpB-deaminase plasmid and the reRNA plasmid are transfected using Lipo 3000 (Thermo Scientific) into HEK293FT cells into a single well of a 96-well plate. The cells are left to incubate for 72 hours before they are harvested for sequencing. Quick Extract DNA solution is used to extract gDNA from cells for subsequent NGS analysis of the targeted loci.

### 17. Example 8: Constructing and Testing a TnpB-RT Fusion

To generate a TnpB RT fusion construct, both a human codon optimized TnpB ORF and a desired human codon optimized RT are amplified from plasmids with primer overhangs containing the desired linker sequence. In addition, the desired nuclear localization signal is encoded on the amplification primers. Two amplified fragments containing the TnpB ORF and RT are stitched together using Gibson Assembly Master Mix (NEB), into the pCDNA3.1 vector (Thermo Scientific). The cognate extended and engineered reRNA for the indicated TnpB ortholog is cloned under the control of the U6 promoter using Gibson Assembly Master Mix (NEB), into the pZ147-BvCas12b-sgRNA-scaffold vector (Addgene). The extended reRNA is designed such that the guide sequence is replaced to match the desired target sequence with a cognate TAM for the indicated TnpB ortholog, particularly the human genome in the case that the construct is to be used in a human genome editing experiment. In addition, an reRNA extension that contains a template for the desired edit, along with a homologous sequence designed to bind to the TnpB non-target strand are included in the engineered reRNA. In experiments where the TnpB is mutated, the desired mutation is created by amplifying the entire plasmid in a single amplicon where the primer encodes the mutation and is cloned using the KLD enzyme mix (NEB). In experiments where a second TnpB ORF is included in the system, it is either cloned into the same construct, as described in Example 1, or expressed from a separate pCDNA3.1 vector. To evaluate the efficacy of the system, 100 ng of each of the TnpB-RT plasmid and the reRNA plasmid are transfected using Lipo 3000 (Thermo Scientific) into HEK293FT cells into a single well of a 96-well plate. The cells are left to incubate for 72 hours before they are harvested for sequencing. Quick Extract DNA solution is used to extract gDNA from cells for subsequent NGS analysis of the targeted loci.

### 18. Example 9: Computational Protocol to Determine 5′ End of an reRNA

The following protocol can be carried out to experimentally determine 5′ end of an reRNA, as depicted in FIG. 1.

Step 1. For each TnpB in Table A, synthesize the genomic locus (500 bp upstream of the first ORF in the TnpB operon all the way to 500 bp downstream of the predicted 3′ end of the reRNA) through a commercially available DNA synthesis vendor (i.e., Twist, Genewiz, etc)

Step 2. Clone the genomic locus into a commonly used bacterial cloning vector, like pUC19

Step 3. Transform lab E. coli with the cloned plasmid from step #2, and grow in liquid culture

Step 4. While in Log phase growth, isolate total RNA from the E. coli using a commercially available kit, e.g., ThermoFisher Product No. 16096020.

Step 5. Design an RT primer that binds (i.e., reverse complement) somewhere maximum 50 bp upstream (5′) of the predicted 3′end of the reRNA

Step 6. Use #4 and #5 as inputs into a standard 5′ RACE protocol, e.g., S′ RACE Protocol using the Template Switching RT Enzyme Mix (NEB #M0466). Rapid amplification of cDNA ends (RACE) is a widely used technique to identify 5′ (5′ RACE) or 3′ (3′ RACE) end of an RNA transcript when its sequence is only partially known. This 5′ RACE protocol contains two steps. In the first step, template switching reverse transcription reaction generates cDNAs with a universal sequence of choice, introduced by a template switching oligo (TSO), attached to the 3′ end of the cDNA (S′ end of the transcript). In the second step, 5′ end of the transcript can be identified via PCR amplification with primers that are specific to the gene of interest and the TSO handle.

Step 7. Sequence products from step #6 using next-generation sequencing (Illumina) technology

Step 8. Map sequencing products onto synthesized genomic locus from #1, with the most abundant 5′ ends representing a putative 5′ bound for the reRNA.

Step 9. Experimentally test cutting activity by combining the reRNA with putative 5′ bounds (including up to 3′ bound and an extension encoding the 16-24nt DNA-targeting spacer), cognate TnpB protein, and DNA substrate.

As an alternative to Steps 1-7, the method of Karvelis et al. (Nature, 2021) shown in FIG. 1(c) and FIG. 1(d) may be used to experimentally determine 5′ end of an reRNA. In that approach, the authors co-transform the genomic locus comprising the IS (which includes the TnpB gene) with a His -tagged TnpB, and subsequently pull down the His -tagged TnpB protein and sequence all small RNAs that are associated with the protein. The below approach and the method of Karvelis et al. demonstrate there are a variety of methods one might take to identify 5′ end of any given reRNA.

### 19. Example 10. Demonstration of Precise Editing of TnpB Delivered In Vitro by Plasmid

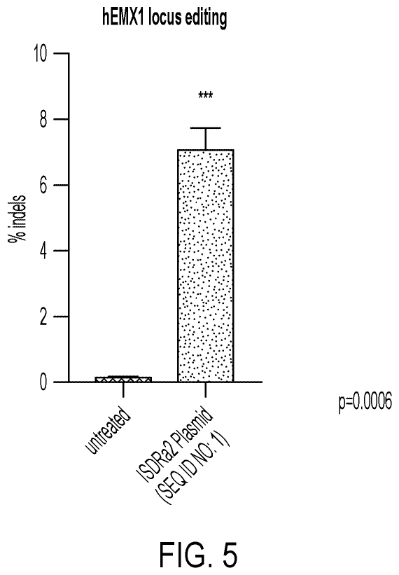

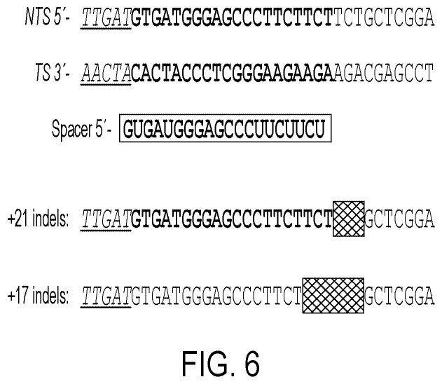

FIG. 5 shows the results testing ISDra2 TnpB endonuclease activity in an all-in-one plasmid based system delivered to HEK293T cells by Lipofectamine. Here, the nuclease is driven by a CMV promoter (followed by a T2A GFP) and the guide RNA (TnpB reRNA scaffold and spacer targeting EMX1) is driven by a U6 promoter. After transfection, cells were harvested 72 hours later in lysis buffer (as detailed in methods). Cells were analyzed by next-generation sequencing (NGS) and determined to have significantly more indels when treated with the plasmid encoding TnpB and the guide RNA using an unpaired t test (p=0.0006). FIG. 6 shows the most common indels detected by NGS.

### 20. Example 11. Demonstration of Precise Editing of TnpB Gene Editing System Delivered In Vivo in Mouse Model by LNP Composition

This example demonstrates precise editing with a TnpB gene editing system in vivo with LNP delivery using Table (III)Compound C59.

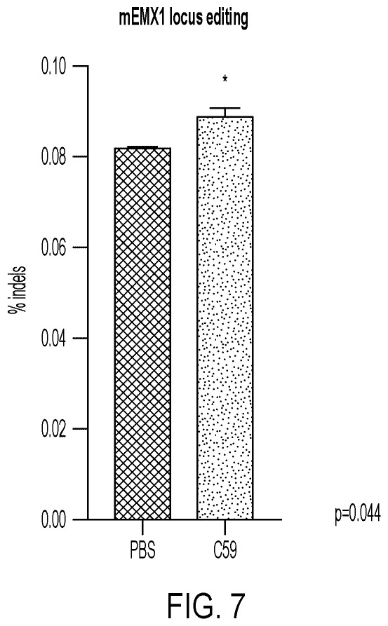

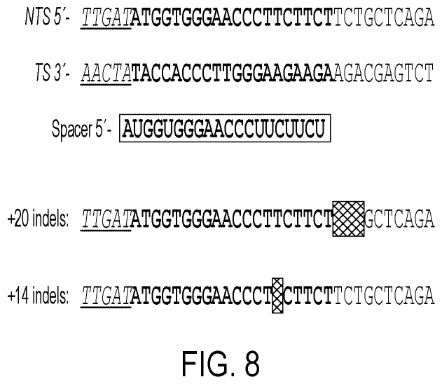

FIG. 7 shows the results testing ISDra2 TnpB endonuclease activity in an RNA LNP system in vivo using C57BL/6 mice. Here, the RNA encoding the nuclease was human codon optimized. The TnpB ncRNA (an engineered TnpB reRNA comprising an EMX1-targeting guide RNA) was produced via in vitro transcription (IVT). The two components ((1) mRNA encoding the TnpB and (2) ncRNA) were then assembled in LNP C59 at a ratio of 1:1 and subsequently quality controlled for encapsulation efficiency, particle size, polydispersity, and endotoxins. Formulated RNA LNPs were then injected into male 8 week old C57BL/6 mice via IV single dose injection (n=4). Untreated animals were injected with PBS (n=2). After 7 days, animals were sacrificed and livers were flash frozen. After genomic DNA (gDNA) extraction, DNA was analyzed by next-generation sequencing (NGS) and determined to have significantly more indels (i.e., evidence of precision editing) when treated with the LNP C59 encoding TnpB and the TnpB ncRNA compared to PBS using an unpaired t test (p=0.0444). FIG. 8 shows the two most common indels created at the mouse EMX1 locus using NGS.

### Example 11 Methods and Materials

### Nanoparticle Formulation Procedure

Ionizable lipids, DSPC, cholesterol, and PEG2k-DMG were dissolved in pure ethanol at a 48.5:10:39:2.5 mol % ratio with a total lipid concentration of 10.8 mM. A 0.40 mg/mL payload solution was prepared using acidic buffer (pH 4.0-5.0) containing the TnpB (SEQ ID NO: 1) and RNA (SEQ ID NO: 384) in a 1:1 ratio by mass. The payload and lipid solutions were mixed at a 3:1 volume ratio using the NanoAssemblr microfluidic system at a 12 mL/min total flow rate resulting in rapid mixing and self-assembly of LNPs. Formulations were further dialyzed against PBS (pH 7.4) overnight at 4° C., concentrated using centrifugal filtration and filtered (0.2 μm pore size). The particle size and polydispersity index (PDI) of formulations was measured by dynamic light scattering (DLS) using a Zetasizer Ultra (Malvern Panalytical). RNA encapsulation efficiency (EE %) was determined by Ribogreen assay.

TABLE 9-1
Lipid nanoparticle formulations

| | | Particle | PDI (scalar | |
|---|---|---|---|---|
| Form. | IL | Size (nm) | units) | EE (%) |
| F-1 | C59 | 91.7 | 0.004 | 98.6 |
| F-2 | CY63 | 64.0 | 0.107 | 98.4 |
| F-3 | 77 | 67.8 | 0.005 | 98.0 |

### Mammalian Cell Culture

HEK293T (ATCC CRF-3216) cells were cultured in Dulbecco's modified Eagle's medium (DMEM) plus GlutaMAX (Thermo Fisher Scientific), supplemented with 10% (v/v) fetal bovine serum (Gibco). Cells were maintained at 37° C. with 5% CO2.

### Genomic DNA Extraction

After incubation, the media was removed from the cells and genomic DNA was extracted by the addition of prepGEM reagent (Thomas Scientific: PUN0050) directly into each well of the tissue culture plate. Lysed cells were transferred to a 96 well PCR plate and incubated at 75° C. for 10 minutes, followed by 95° C. enzyme inactivation step for 5 minutes.

### High-Throughput DNA Sequencing of Genomic DNA Samples

Human EMX1 or mouse EMX1 gene was amplified from genomic DNA samples and sequenced on an Illumina NextSeq. Briefly, amplification primers containing Illumina forward and reverse adapters were used for a first round of PCR (PCR1) amplifying EMX1 targeting site. 25 μl PCR1 reactions were performed with 0.3 μM of each forward and reverse primer, 2 μl of genomic DNA extract and 12.5 μl of KAPA HIFI HOTSTART PCR master mix. PCR reactions were carried out as follows: 95° C. for 3 minutes, then 25 cycles of [98° C. for 20 seconds, 67° C. (human) or 62° C. (mouse) for 15 seconds, and 72° C. for 15 seconds], followed by a final 72° C. extension for 2 minutes. PCR reactions were purified using Ampure XP beads (Beckman Coulter) and eluted in 20 μl H2O. Unique barcoding primer pairs were added to each sample in a secondary PCR reaction (PCR2). Specifically, 25 μl PCR2 reactions were performed with 10 μl of IDT for Illumina UDI primers (Illumina), 2 μl of purified PCR1 reaction, and 12.5 μl of KAPA HIFI HOTSTART PCR master mix. PCR reactions were carried out as follows: 95° C. for 3 minutes, then 10 cycles of [98° C. for 20 seconds, 55° C. for 15 seconds, and 72° C. for 15 seconds], followed by a final 72° C. extension for 2 minutes. PCR2 reactions were purified by SequalPrep Normalization plate kit (Thermo Fisher Scientific) and pooled. Size and purity were evaluated by TapeStation High Sensitivity D1000 assay (Agilent). DNA concentration was measured by fluorometric quantification (Qubit, Thermo Fisher Scientific) and libraries were sequenced with 30% PhiX sequencing control on an Illumina NextSeq 2000 instrument using P1 or P2 600 cycle kit. Sequencing reads were demultiplexed and alignment of amplicon sequences to a reference sequence was performed using CRISPResso2. Editing outcome (InDels %) was calculated as the number of modified reads divided by total aligned reads.

### Plasmid Based Gene Editing

Plasmid encoding ISDRa2 and guide RNA was synthesized by Twist Bioscience. For transfections, 1.5e5 293T cells (ATCC) were transfected using Lipofectamine LTX (Thermofisher: 15338030) according to the manufacturer's protocol. Transfected cells were incubated at 37° C. for 72 hrs before lysis in prepGEM reagent (Thomas Scientific: PUN0050) according to the manufacturer's protocol. gDNA was used for NGS analysis.

### In Vitro Transcription of Guide RNA

Geneblocks encoding the guide RNA were synthesized by Integrated DNA Technologies. T7 promoter was placed upstream of ncRNA sequence which included TnpB Trim 2 reRNA and 20nt spacer. HDV ribozyme sequence was placed downstream of the guide RNA. Geneblock was amplified using Q5® High-Fidelity 2X Master Mix (New England Biolabs). The forward primer used was M13F and the reverse primer used was specific to the HDV ribozyme resulting in 3′ ribozyme protection for the final RNA. 50 pl reactions were performed with 0.3 μM of each forward and reverse primer, 1 μl of geneblock template and 12.5 μl of Q5 High-Fidelity 2×master mix. PCR reactions were carried out as follows: 98° C. for 30 seconds, then 35 cycles of [98° C. for 10 seconds, 63° C. for 30 seconds, and 72° C. for 15 seconds], followed by a final 72° C. extension for 2 minutes. IVT template was purified using GeneJET PCR Purification Kit (Thermo Fisher Scientific). 40 μl IVT reactions were prepared using HiScribe T7 High Yield RNA Synthesis Kit (New England Biolabs). Specifically, 4 μL T7 RNA Polymerase Mix and 2 μL 10×reaction buffer were combined with 5 mM each ATG, GTP, CTP. N1-Methyl-Pseudouridine-5′-Triphosphate (Trilink) was also added at 5 mM. 4 mM TriLink CleanCap® Reagent AG (3′OMe) was added (Trilink). Finally, lug linear Template DNA was added and the synthesis reaction was carried out at 37° C. for 2 hours. The RNA was immediately purified using Monarch RNA Cleanup Kit (New England Biolabs). RNA concentration was measured by Nanodrop (Thermo Fisher) and purity and size were evaluated by RNA ScreenTape analysis on Tape station (Agilent).

### Primers Used for NGS

Mouse fwd:
(SEQ ID NO: 382)
AGGTGAAGGTGTGGTTCCAG
Mouse rev:
(SEQ ID NO: 383)
TAGTCATTGGAGGTGACATCA
Human fwd:
(SEQ ID NO: 384)
AGGTGAAGGTGTGGTTCCAG
Human rev:
(SEQ ID NO: 385)
TAGTCATTGGAGGTGACATCG
TnpB spacer/guide sequence targeting mouse EMX1:
(SEQ ID NO: 386)
ATGGTGGGAACCCTTCTTCT
TnpB spacer/guide sequence targeting human EMX1:
(SEQ ID NO: 387)
GTGATGGGAGCCCTTCTTCT
Full DNA sequence for IVT reaction (to create
the TnpB ncRNA targeting mouse EMX1):
(SEQ ID NO: 388)
GTAAAACGACGGCCAGTTAATACGACTCACTATAAGGATAATGGTGGCT
GCGGGAATCTCAGACACCTTAAACGCTCATGGAGGCTATGAAAATGGTC
TGCGAAGTGAGAATCACGCGACTTTAGTCGTGTGAGGTTCAAATGGTGG
GAACCCTTCTTCTGGCCGGCATGGTCCCAGCCTCCTCGCTGGCGCCGGC
TGGGCAACATGCTTCGGCATGGCGAATGGGACTGAAGAGCGGTCATAGC
TGTTTCCTG

### Mouse Study

C57BL/6 8 week old male mice were purchased from JAX. Animals were dosed via IV injection at 2.0 mg/kg. n=4 for LNP injected animals and n=2 for PBS treated animals. After 7 days animals were sacrificed and the livers were snap frozen for further analysis.

ISDRa2 protein sequence
(SEQ ID NO: 1)
MIRNKAFVVRLYPNAAQTELINRTLGSARFVYNHFLARRIAAYKESGKGLTYGQTSS
ELTLLKQAEETSWLSEVDKFALQNSLKNLETAYKNFFRTVKQSGKKVGFPRFRKKR
TGESYRTQFTNNNIQIGEGRLKLPKLGWVKTKGQQDIQGKILNVTVRRIHEGHYEAS
VLCEVEIPYLPAAPKFAAGVDVGIKDFAIVTDGVRFKHEQNPKYYRSTLKRLRKAQQ
TLSRRKKGSARYGKAKTKLARIHKRIVNKRQDFLHKLTTSLVREYEIIGTEHLKPDN
MRKNRRLALSISDAGWGEFIRQLEYKAAWYGRLVSKVSPYFPSSQLCHDCGFKNPE
VKNLAVRTWTCPNCGETHDRDENAALNIRREALVAAGISDTLNAHGGYVRPASAG
NGLRSENHATLVVSRADPKKKRKVEFGSG*
ISDRa2 codon optimized nucleotide sequence (RNA produced by Vernal)
(SEQ ID NO: 383)
GGGAAATAAGAGAGAAAAGAAGAGTAAGAAGAAATATAAGACCCCGGCGCCGC
CACCATGATCCGGAACAAGGCTTTTGTTGTCCGGCTGTACCCCAACGCCGCTCAG
ACCGAGCTGATCAACAGAACACTTGGGTCTGCCAGGTTCGTGTACAACCACTTCC
TGGCCCGAAGAATCGCCGCATATAAGGAAAGCGGCAAAGGCCTGACCTACGGCC
AGACAAGCAGCGAGCTGACCCTGCTGAAACAGGCCGAGGAAACCAGCTGGCTG
AGCGAGGTGGATAAGTTCGCCCTGCAGAACAGCCTGAAGAACCTGGAAACCGCC
TACAAGAATTTCTTCAGAACAGTGAAGCAGAGCGGCAAGAAAGTGGGATTTCCC
AGATTTAGAAAGAAGCGGACAGGCGAGAGCTACAGGACACAGTTCACCAACAA
CAACATCCAAATCGGCGAGGGCAGACTCAAACTGCCTAAGCTGGGCTGGGTCAA
GACAAAGGGACAGCAAGACATCCAGGGCAAGATCCTGAACGTGACCGTGCGGA
GAATCCATGAAGGCCACTACGAGGCCAGCGTGCTGTGCGAGGTGGAAATCCCCT
ACCTGCCTGCCGCCCCTAAGTTCGCTGCCGGCGTGGACGTGGGCATCAAGGACTT
CGCCATCGTGACCGACGGCGTGAGATTCAAGCACGAGCAGAATCCTAAATACTA
CAGAAGCACCCTGAAGAGACTGAGAAAAGCCCAGCAGACCCTGTCTAGACGGA
AAAAGGGCAGTGCTAGATACGGCAAGGCCAAGACCAAGCTGGCCAGAATCCAC
AAGAGAATTGTGAACAAGCGCCAAGATTTCCTGCACAAGCTGACAACAAGCCTG
GTGCGGGAATACGAGATCATCGGCACCGAGCACCTGAAGCCTGACAACATGAGA
AAAAATCGGAGACTCGCCCTGAGCATCAGCGACGCCGGCTGGGGAGAATTCATC
AGACAGCTGGAGTACAAGGCCGCTTGGTATGGCAGACTGGTCAGCAAGGTGTCC
CCTTACTTCCCATCCAGCCAGCTGTGTCACGATTGCGGCTTTAAGAACCCAGAAG
TGAAGAACCTGGCCGTGCGGACTTGGACCTGTCCTAACTGCGGCGAGACCCACG
ACAGAGATGAGAACGCCGCTCTGAACATCAGACGGGAAGCCCTCGTGGCCGCTG
GAATTTCTGACACCCTGAATGCCCACGGCGGCTACGTGCGCCCCGCCTCTGCCGG
AAATGGACTGCGGAGCGAAAACCACGCCACCCTGGTGGTGTCCAGAGCCGACCC
TAAGAAAAAACGGAAGGTTGAGTTCGGCTCCGGCTAACTAGTGACTGACTAGGA
TCTGGTTACCACTAAACCAGCCTCAAGAACACCCGAATGGAGTCTCTAAGCTACA
TAATACCAACTTACACTTACAAAATGTTGTCCCCCAAAATGTAGCCATTCGTATC
TGCTCCTAATAAAAAGAAAGTTTCTTCACAT
Full TnbB ncRNA sequence used for human EMX1 editing studies
(SEQ ID NO: 384)
GATTCAAGAATCCCGAAGTGAAGAATCTTGCCGTCCGTACATGGACTTGCCCGA
ACTGTGGGGAAACCCATGACCGAGACGAGAACGCTGCGCTGAACATTCGGCGTG
AAGCGTTGGTGGCTGCGGGAATCTCAGACACCTTAAACGCTCATGGAGGCTATG
TCAGACCTGCTTCGGCGGGCAATGGTCTGCGAAGTGAGAATCACGCGACTTTAGT
CGTGTGAGGTTCAAGTGATGGGAGCCCTTCTTCT
ISDRa2 TnpB_T2A_GFP DNA sequence used for plasmid studies
(SEQ ID NO: 385)
ATGATAAGGAATAAGGCTTTCGTGGTCAGGCTGTACCCAAATGCGGCTCAGACT
GAACTGATTAACCGCACGCTGGGTAGCGCAAGGTTCGTCTACAACCACTTCCTTG
CCCGTCGCATTGCGGCCTACAAGGAAAGCGGGAAGGGACTGACCTACGGGCAAA
CGAGTAGCGAACTGACCCTTCTGAAGCAGGCTGAAGAAACCTCCTGGCTCTCGG
AAGTAGATAAGTTTGCTTTGCAGAACTCGCTGAAAAACCTTGAGACCGCGTACA
AGAACTTCTTTCGGACTGTGAAGCAGTCCGGTAAAAAGGTAGGATTCCCACGTTT
CAGAAAGAAGCGCACGGGAGAGTCCTACCGGACTCAATTCACCAACAACAACAT
CCAAATTGGGGAAGGTAGGCTCAAACTTCCTAAGCTGGGATGGGTGAAAACCAA
GGGCCAGCAGGATATTCAAGGGAAGATTCTGAATGTCACTGTGCGCCGTATTCA
CGAAGGCCATTACGAAGCGTCCGTTCTCTGTGAAGTCGAGATTCCCTACCTGCCT
GCGGCTCCCAAGTTTGCAGCGGGTGTGGATGTCGGCATCAAGGATTTTGCCATCG
TGACCGATGGCGTGAGGTTTAAGCATGAACAGAATCCGAAATATTACCGCTCCA
CCCTGAAAAGACTTCGTAAAGCTCAGCAAACCCTGTCCAGACGGAAGAAGGGCA
GCGCACGTTACGGGAAAGCGAAAACCAAGCTGGCTCGGATTCACAAGCGCATTG
TCAATAAGCGTCAGGATTTCCTTCACAAGCTCACCACCTCCCTGGTGCGTGAGTA
CGAAATCATCGGAACCGAACACCTTAAACCCGACAACATGCGGAAAAATCGCCG
CCTTGCACTGAGCATCAGTGATGCGGGCTGGGGTGAGTTCATCCGGCAGTTGGA
ATACAAGGCAGCGTGGTACGGGCGACTGGTATCTAAAGTCAGCCCCTACTTTCCA
TCTAGCCAGTTGTGTCATGACTGCGGATTCAAGAATCCCGAAGTGAAGAATCTTG
CCGTCCGTACATGGACTTGCCCGAACTGTGGGGAAACCCATGACCGAGACGAGA
ACGCTGCGCTGAACATTCGGCGTGAAGCGTTGGTGGCTGCGGGAATCTCAGACA
CCTTAAACGCTCATGGAGGCTATGTCAGACCTGCTTCGGCGGGCAATGGTCTGCG
AAGTGAGAATCACGCGACTTTAGTCGTGAGCAGGGCCGACCCAAAGAAGAAGCG
GAAGGTCGAATTCGGCAGTGGAGAGGGCAGAGGAAGTCTGCTAACATGCGGTGA
CGTCGAGGAGAATCCTGGCCCAGTGAGCAAGGGCGAGGAGCTGTTCACCGGGGT
GGTGCCCATCCTGGTCGAGCTGGACGGCGACGTAAACGGCCACAAGTTCAGCGT
GTCCGGCGAGGGCGAGGGCGATGCCACCTACGGCAAGCTGACCCTGAAGTTCAT
CTGCACCACCGGCAAGCTGCCCGTGCCCTGGCCCACCCTCGTGACCACCCTGACC
TACGGCGTGCAGTGCTTCAGCCGCTACCCCGACCACATGAAGCAGCACGACTTCT
TCAAGTCCGCCATGCCCGAAGGCTACGTCCAGGAGCGCACCATCTTCTTCAAGGA
CGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGG
GCACAAGCTGGAGTACAACTACAACAGCCACAACGTCTATATCATGGCCGACAA
GCAGAAGAACGGCATCAAGGTGAACTTCAAGATCCGCCACAACATCGAGGACGG
CAGCGTGCAGCTCGCCGACCACTACCAGCAGAACACCCCCATCGGCGACGGCCC
CGTGCTGCTGCCCGACAACCACTACCTGAGCACCCAGTCCGCCCTGAGCAAAGA
CCCCAACGAGAAGCGCGATCACATGGTCCTGCTGGAGTTCGTGACCGCCGCCGG
GATCACTCTCGGCATGGACGAGCTGTACAAGTAA

### Additional TnpB Proteins that May be Formulated for Editing Pursuant to Example 11:

TABLE A
any protein
Geobacillus stearothermophilus (GST) TnpB
Protein
MYFCIKQQLNGLTKEEYLTLRELCHIAKNMYNVGLYNVRQYYFEHKEFLNYEKNY
HLAKTNENYKLLNSNMAQQILKKVNEAFKSFFGLISLAKQGKYDHKAISIPKYLKKD
GFHSLIIGQIRIDGNKFTIPYSRLFKKTHKPITITIPPVLLDKKIKQIEIIPKHHARFFEIQY
KYEMPEDQRELNDQKALAIDLGLNNLATCVTSDGRSFIIDGRRLKSINQWFNKENAR
LQSIKDKQKIKGTTRKQALLAMNRNNKVNDYINKTCRYIINYCIENQIGKLVIGYAET
WQRNMNLGKKTNQNFVNIPLGNIKEKLEYLCEFYGIEFLKQEESYTSQASFFDGDEIP
EYNADNPKEYKFSGKRIKRGLYRTKSGKLINADVNGALNILKKSKAVDLSVLCSSGE
VDTPQRIRIASRADPKKKRKV* (SEQ ID NO: 386)
Codon optimized nucleotide sequence
ATGTACTTCTGCATCAAGCAGCAGCTGAACGGCCTGACCAAGGAGGAGTATCTG
ACCCTGAGGGAGCTGTGTCACATCGCCAAGAACATGTACAACGTGGGCCTGTAC
AATGTGAGGCAGTACTACTTTGAGCACAAGGAGTTCCTGAACTACGAGAAGAAC
TACCACCTCGCCAAGACCAACGAGAACTACAAGCTGCTGAACAGCAACATGGCC
CAGCAGATCCTGAAGAAGGTGAACGAGGCCTTCAAGTCCTTCTTCGGCCTGATCT
CCCTGGCCAAGCAGGGCAAGTACGATCACAAGGCCATCAGCATCCCCAAGTACC
TGAAGAAGGACGGCTTCCACAGCCTGATCATCGGCCAGATCAGGATCGACGGCA
ACAAGTTCACCATCCCCTACAGCCGCCTGTTCAAGAAGACCCACAAGCCCATCAC
CATCACCATCCCCCCCGTGCTGCTTGATAAGAAGATCAAGCAGATCGAGATCATC
CCCAAGCACCACGCCAGATTCTTCGAGATCCAGTACAAGTACGAGATGCCGGAG
GATCAGCGGGAGCTGAATGATCAGAAGGCCCTGGCCATCGACCTGGGCCTGAAC
AATCTGGCCACATGCGTGACCAGCGATGGCAGAAGCTTCATCATCGACGGCAGG
CGGCTGAAGTCCATCAATCAGTGGTTCAACAAGGAGAACGCCCGGCTGCAGTCC
ATCAAGGACAAGCAGAAGATCAAGGGCACCACCCGGAAGCAGGCCCTGCTGGC
CATGAACAGGAACAACAAGGTGAACGACTACATCAACAAGACCTGCAGGTACAT
CATCAACTACTGCATCGAGAACCAGATCGGCAAGCTGGTGATCGGCTATGCCGA
GACATGGCAGAGAAACATGAATCTCGGCAAGAAGACCAACCAGAACTTCGTGAA
CATCCCCCTGGGGAACATCAAGGAGAAGCTGGAGTACCTGTGCGAGTTCTACGG
CATCGAGTTCCTGAAGCAGGAGGAGAGCTACACAAGCCAGGCCTCCTTCTTCGA
CGGGGATGAGATCCCCGAGTACAACGCCGACAACCCCAAGGAGTACAAGTTCAG
CGGCAAGCGCATTAAGCGCGGGCTGTACCGGACCAAGTCCGGCAAGCTGATCAA
CGCCGACGTGAACGGCGCCCTGAACATCCTGAAGAAGTCCAAGGCCGTGGACCT
GAGCGTGCTGTGTAGCAGCGGCGAGGTGGATACCCCTCAGAGGATCAGAATCGC
CTCCAGAGCCGACCCCAAGAAGAAGCGGAAGGTCTGA (SEQ ID NO: 387)
TnpB ncRNA
TTAAGCGCGGCTTGTATCGAACAAAGTCTGGCAAACTAATTAATGCTGATGTCAA
TGGCGCATTAAACATCTTAAAGAAAAGTAAAGCTGTAGACCTGAGTGTCTTATGC
TCTAGCGGCGAAGTGGACACGCCTCAAAGAATAAGGATTGCTTGAAGCAGTCAA
ACTTCTTTGGAAGCCCCCACTTCAAATTTTCGCTAGAAAATTAAGTGGGGGTAGT
TCACGGACAAGTGGTTCACCATGCGTTGCTTTATGGTATGATAG (SEQ ID NO: 388)
Helicobacter pylori (Hpy)TnpB
Protein
MLITYKQKLYKNDKNRRIDTLLRRYGVFYNHCIALHKRYYRLFKKYLKLNDLQKHT
TKLKKTHRYAFLKTLGSQTLQDLTERIDKAFKKFFKKQAKLPRFKKVANYKSFTFKS
QADKKTGLNKGVGFEIKDNVVSFNGYSYKFIKTYALIGKVKTLTIKRDNMGDYFLCL
VCELENNPNKQTACDKSVGFDFGLRTFLTGSDNTKIESPLFFSQYLPLIKRLSKNLSK
KVKGSNNFKKAKKKLAQLHQKIKHLRTDFFYKLALKLSKKYQSIFIEDLNMKAMQK
LWGRKVSDIAFSEFVKILENKAHVVKIDRFYPSSKTCSNCLSVDENFNKDIKKLGKTD
KEREYHCKYCGLELDRDLNASINIHRVGASILGVEFVRPTSRADPKKKRKV* (SEQ ID
NO: 389)
Codon optimized nucleotide sequence
ATGCTGATCACCTACAAGCAGAAGCTGTACAAGAACGACAAGAACAGGCGGATC
GACACCCTGCTGCGGCGGTACGGCGTGTTCTACAACCACTGTATCGCCCTGCACA
AGCGGTACTACCGGCTGTTCAAGAAGTACTTGAAGCTGAACGATCTGCAGAAGC
ACACCACCAAGCTGAAGAAGACCCACAGGTACGCCTTCCTGAAGACCCTGGGCA
GCCAGACCCTGCAGGATCTGACAGAGCGGATCGACAAGGCCTTCAAGAAGTTCT
TCAAGAAGCAGGCCAAGCTGCCCAGGTTCAAGAAGGTGGCCAACTACAAGAGCT
TCACCTTCAAGTCTCAGGCCGACAAGAAGACCGGCCTGAACAAGGGCGTGGGCT
TCGAGATCAAGGACAATGTGGTGAGCTTCAACGGCTACAGCTACAAGTTCATCA
AGACCTACGCCCTGATCGGCAAGGTGAAGACCTTGACCATCAAGCGGGACAACA
TGGGAGACTACTTCCTGTGTCTGGTGTGCGAGCTGGAGAACAATCCAAACAAGC
AGACCGCCTGCGATAAGAGCGTGGGCTTCGACTTCGGCCTGAGAACCTTCCTGAC
CGGCTCCGACAACACCAAGATCGAGAGCCCCCTGTTCTTCAGCCAGTATCTGCCT
CTGATCAAGAGGCTGAGCAAGAACCTGAGCAAGAAGGTGAAGGGCAGCAACAA
CTTCAAGAAGGCCAAGAAGAAGCTGGCCCAGCTGCACCAGAAGATCAAGCACCT
GAGGACCGACTTCTTCTACAAGCTGGCCCTGAAGCTGAGCAAGAAGTACCAGAG
CATCTTCATCGAGGACCTGAACATGAAGGCCATGCAGAAGCTGTGGGGCAGGAA
GGTGAGCGACATCGCCTTCTCCGAGTTCGTGAAGATCCTGGAGAACAAGGCCCA
CGTGGTGAAGATCGACCGGTTCTACCCCAGCAGCAAGACCTGCAGCAACTGCCT
GTCCGTGGACGAGAACTTCAACAAGGACATCAAGAAGCTGGGGAAGACCGACA
AGGAGCGGGAGTACCACTGCAAGTACTGCGGCCTGGAGCTGGACAGAGACCTGA
ACGCCAGCATCAACATCCACCGCGTGGGCGCCTCTATCCTGGGCGTGGAGTTCGT
GAGGCCCACCTCCAGAGCCGACCCCAAGAAGAAGAGGAAGGTGTGA (SEQ ID
NO: 390)
TnpB ncRNA for Helicobacter pylori (Hpy)TnpB
ATTCATAGGGTTGGGGCATCAATCCTTGGGGTAGAATTTGTAAGACCTACCTAGT
AGGCTGAGTTTGCTTGATCCCAATTTTTCTCATGCTTTAGCTAGAATCCCCTAGCT
TTAGCTATGGGGAGTATGTCAA (SEQ ID NO: 391)
Deinococcus murrayi (Dmu) TnpB
Protein
MESTTAPKRNKAFVIRLYPNKAQAERINRTLGCARFVYNHFLARRIETYRQDGKGM
TYAATDRALTLLKREEGTAWLAEVDKFALQQSLRDLERAYQNFFRTVKKSGKKVG
FPKFKKKRTGEAYRTQFTNNNIEIGKGCIKLPKLGWVKTRGQRDMQGKILNVTVRR
VHEGHYEASVLCEVEIPYLPAAPRFAAGVDVGIKDFAIVTDGEGEFTHHANPKYYRN
GLKKLRKAQKTLSRRKKGSARYGKAKTKLARIHKRVANKRQDFIHKLTTALVREYE
ITCTEHLKPDNMRKNRRLALSISDAGWGEFIRQLEYKATWYGRLVSKISPHFPSSQM
CHDCGSLNPAVKNLAVREWTCPNCGETHDRDENAALNIRREGLVAAGISDTQNARR
ECVSPAPAGNVRSRADPKKKRKV* (SEQ ID NO: 392)
Codon optimized nucleotide sequence
ATGGAGAGCACCACCGCCCCCAAGAGGAACAAGGCCTTCGTGATCCGCCTGTAC
CCCAACAAGGCCCAGGCCGAGAGAATCAATCGGACCCTGGGCTGCGCCAGGTTC
GTGTACAACCACTTCCTGGCCAGGCGGATCGAGACCTACAGGCAGGACGGCAAA
GGGATGACCTATGCCGCCACCGATAGGGCCCTGACCCTGCTGAAGAGGGAAGAG
GGCACAGCTTGGCTGGCCGAAGTGGACAAGTTCGCCCTGCAGCAGTCCCTGAGG
GACCTGGAGCGGGCCTACCAGAACTTCTTCCGGACTGTGAAGAAGTCCGGCAAG
AAGGTGGGCTTCCCCAAGTTCAAGAAGAAGCGGACTGGCGAGGCCTACAGGACC
CAGTTCACCAACAACAACATCGAGATCGGCAAGGGCTGCATCAAGCTGCCCAAG
CTGGGCTGGGTGAAGACCAGGGGCCAGAGGGACATGCAGGGCAAGATCCTGAA
CGTGACCGTGCGCAGAGTGCACGAGGGCCACTATGAGGCCAGCGTGCTGTGTGA
GGTGGAGATCCCTTATCTGCCTGCCGCCCCTAGATTCGCCGCCGGCGTGGATGTG
GGAATCAAGGACTTCGCCATCGTGACCGATGGCGAGGGCGAGTTCACCCACCAC
GCCAATCCTAAGTACTACAGGAACGGCCTGAAGAAGCTGAGGAAGGCTCAGAAG
ACCCTGAGCCGGCGGAAGAAGGGCTCCGCCAGATACGGCAAGGCCAAGACCAA
GCTGGCCCGCATTCACAAGCGGGTGGCCAACAAGCGGCAGGACTTCATCCACAA
GCTGACCACAGCCCTGGTGAGGGAGTACGAGATCACCTGCACCGAGCACCTGAA
GCCCGACAACATGCGGAAGAACAGGAGACTGGCCCTGAGCATCAGCGATGCCGG
CTGGGGCGAGTTTATCAGGCAGCTGGAGTACAAGGCTACCTGGTACGGCAGGCT
GGTGAGCAAGATCAGCCCCCACTTCCCCAGCAGCCAGATGTGTCACGACTGCGG
CAGCCTGAACCCCGCCGTGAAGAATCTGGCCGTGAGGGAGTGGACCTGCCCTAA
TTGTGGGGAGACCCACGACAGGGACGAGAACGCCGCCCTGAACATCAGAAGAG
AGGGCCTGGTGGCCGCCGGCATCTCTGACACCCAGAATGCCAGAAGAGAGTGTG
TGAGCCCTGCCCCTGCCGGCAATGTGAGAAGCAGAGCCGATCCTAAGAAGAAGC
GGAAGGTGTGA (SEQ ID NO: 393)
reRNA for Deinococcus murrayi (Dmu) TnpB
GTGAAGAATCTTGCCGTCCGCGAATGGACTTGCCCGAACTGCGGGGAAACCCAT
GACCGAGACGAGAACGCCGCGCTGAACATCCGGCGTGAAGGACTGGTGGCCGCA
GGGATTTCGGACACCCAAAACGCCCGTCGAGAATGTGTAAGCCCAGCTCCGGCT
GGCAACGTTCGCTGAAGCGGGAATCCCGCGACTTCAGTCGTGGGAGGTTCAA
(SEQ ID NO: 394)
Alicyclobacillus macrosporangiidus (Ama) TnpB
Protein
MGRKRALIVLNDLCYNNGMNLTLMVKLLPTTEQHQALLETMERFNAACNAIAEVA
FEHRTANKIRLQKLVYDSIRKEFGLSAQMTVRAIAKACEAYKRDKSIKPTFKPHGAIV
YDQRLLSWKGLDRVSILTLGGRILVPILFGEYQAARLQRIRGQADLIYRDGTFYLAVV
VDVPDPPQGAPNGFLGVDLGIKNIATDSDGEVFSGGHVNGLRHRHARLRQRLQSKG
TKSAKRLLKKRRRKEARFATNVNHRIAKVLVAKAKDTGRGIALEDLKGIRDRITVRK
AQRRTQHSWAFHQLRFFIEYKARLAGVPVVFVDPRNTSRTCPSCGHADKRNRPTRD
NFECVECGFAGPADTIAAVNIRRRAEVMQPYAVSRADPKKKRKV* (SEQ ID NO:
395)
Codon optimized nucleotide sequence
ATGGGCCGGAAGAGGGCCCTGATTGTGCTGAACGATCTGTGCTACAACAACGGC
ATGAACCTGACCCTGATGGTCAAGCTGCTGCCCACCACAGAGCAGCATCAGGCC
CTGCTGGAGACCATGGAGCGGTTCAATGCCGCTTGCAATGCCATTGCCGAGGTG
GCCTTCGAGCACAGGACCGCCAACAAGATCCGGCTGCAGAAGCTGGTGTACGAC
AGCATCCGCAAGGAGTTCGGCCTGTCCGCCCAGATGACTGTGAGAGCCATCGCC
AAGGCCTGTGAGGCCTACAAGAGGGACAAGAGCATCAAGCCTACCTTCAAGCCT
CACGGCGCCATCGTGTACGACCAGAGACTGCTGTCCTGGAAGGGCCTGGACAGG
GTGAGCATCCTGACCCTGGGCGGCAGAATCCTGGTGCCTATCCTGTTTGGCGAGT
ACCAGGCCGCCAGACTGCAGAGGATCAGGGGCCAGGCCGATCTGATCTACCGGG
ATGGCACCTTCTACCTGGCCGTGGTGGTGGATGTGCCAGATCCACCTCAGGGCGC
CCCTAATGGCTTCCTGGGCGTGGATCTGGGCATCAAGAACATCGCCACCGACAG
CGACGGCGAGGTGTTCTCTGGCGGCCACGTGAATGGCCTGAGGCACAGACATGC
CAGGCTGAGACAGAGACTGCAGTCTAAGGGCACCAAGAGCGCCAAGAGACTGCT
GAAGAAGAGAAGAAGGAAGGAGGCCAGGTTCGCCACCAACGTGAACCACCGGA
TCGCCAAGGTCCTGGTGGCCAAAGCCAAGGATACCGGCAGAGGCATTGCCCTGG
AGGACCTGAAGGGCATCAGGGACAGGATCACCGTGAGAAAGGCCCAGCGCAGA
ACCCAGCACAGCTGGGCCTTTCACCAGCTGCGGTTCTTCATCGAGTACAAGGCCA
GACTGGCCGGCGTGCCTGTGGTGTTCGTGGACCCCAGGAACACCAGCAGAACCT
GCCCTTCTTGCGGCCACGCCGACAAGAGGAACAGACCCACCAGGGACAACTTCG
AGTGCGTGGAGTGCGGCTTCGCCGGCCCTGCCGATACAATCGCCGCCGTGAATAT
CAGGAGAAGGGCCGAGGTGATGCAGCCCTACGCCGTGAGCAGGGCCGACCCTAA
GAAGAAGCGCAAGGTGTGA (SEQ ID NO: 396)
reRNA for Alicyclobacillus macrosporangiidus (Ama) TnpB
TTGAGTGTGTCGAATGTGGGTTCGCTGGCCCAGCCGACACCATCGCTGCGGTGAA
CATTCGCCGTAGGGCTGAAGTCATGCAGCCGTACGCGGTATAGCGCAGGCTACA
ACTGCAAGCCCCGCCCTTTAGGGCTGGGGTTCATGAC (SEQ ID NO: 397)

1. A pharmaceutical composition comprising:
a) at least one lipid nanoparticle (LNP) comprising at least one ionizable lipid selected from those listed in Tables (I), (II), (III), (IV) or (V); and
b) at least one TnpB gene editing system.

2.-7. (canceled)

8. The pharmaceutical composition of claim 1, wherein the at least one TnpB gene editing system comprises:
a) a nucleic acid sequence encoding a TnpB protein or functional variant thereof, and
b) a TnpB ncRNA or a nucleic acid sequence encoding same, wherein the ncRNA comprises an engineered guide.

9. The pharmaceutical composition of claim 8, wherein the TnpB protein comprises a TnpB protein of Table A or functional fragment thereof, or an amino acid sequence having at least 85%, 90%, 95%, 99%, or up to 100% sequence identity with any of the TnpB protein of Table A, and/or wherein the TnpB ncRNA comprises a nucleic acid sequence from Table B or functional fragment thereof, or a nucleic acid sequence having at least 85%, 90%, 95%, 99%, or up to 100% sequence identity with any nucleic acid sequence from Table B, and/or wherein component a) is a coding RNA and b) is a TnpB ncRNA, and/or wherein the coding RNA is a linear mRNA or a circular mRNA, and/or wherein the TnpB ncRNA comprises one or more chemical modifications selected from 2′-O-Me, 2′-F, and 2′F-ANA at 2′OH; 2′F-4′-Cα—OMe and 2′, 4′-di-Cα—OMe at 2′ and 4′ carbons; phosphodiester modifications comprising sulfide-based Phosphorothioate (PS) or acetate-based phosphonoacetate alterations;
combinations of the ribose and phosphodiester modifications; locked nucleic acid (LNA), bridged nucleic acids (BNA), S-constrained ethyl (cEt), and unlocked nucleic acid (UNA); modifications to produce a phosphodiester bond between the 2′ and 5′ carbons (2′, 5′-RNA) of adjacent RNAs; and a butane 4-carbon chain link between adjacent RNAs.

10.-12. (canceled)

13. The pharmaceutical composition of claim 8, wherein the TnpB gene editing system further comprises a donor DNA template capable of modifying a target sequence.

14. The pharmaceutical composition of claim 13, wherein the donor DNA template is comprises double-stranded DNA, or wherein the donor DNA template comprises single-stranded DNA, or wherein the donor DNA template comprises circular single-stranded DNA, or wherein the donor DNA template comprises an edit flanked by regions of homology to the regions upstream and downstream of a TnpB cut site.

15.-17. (canceled)

18. The pharmaceutical composition of claim 1, wherein the TnpB editing system is capable of editing, modifying or altering a polynucleotide sequence, or is capable of installing an edit at a target site.

19. The pharmaceutical composition of claim 18, wherein the edit comprises a double-strand cut, or comprises an insertion of 1 or more nucleobases, a deletion of 1 or more nucleobases, or a combination thereof, or comprises a transversion edit, or comprises a transition edit, or converts a T←→Cor A←→G, or converts a T→A or G, C→G or A, A→Tor C, or G→C or T.

20.-24. (canceled)

25. The pharmaceutical composition of claim 19, wherein the insertion or deletion is of comprises a whole exon or intron of a gene, or wherein the insertion or deletion comprises a whole or partial gene.

26. (canceled)

27. The pharmaceutical composition of claim 1, wherein the TnpB gene editing system further comprises an accessory protein or a nucleotide sequence encoding the accessory protein.

28. The pharmaceutical composition of claim 27, wherein the accessory protein comprises a nuclease, a deaminase, a recombinase, a reverse transcriptase, or an integrase, and/or wherein the accessory protein is fused to a TnpB protein to form a fusion protein, and/or wherein the TnpB protein comprises a fusion protein which comprises a TnpB protein and a deaminase, or comprises a TnpB protein and a reverse transcriptase, or comprises a TnpB protein and a recombinase, or comprises a TnpB protein and a nuclease, or comprises a TnpB protein and an integrase.

29.-34. (canceled)

35. The pharmaceutical composition of claim 1 for ex vivo delivery, or for in vivo delivery, or wherein the TnpB gene editing system recognizes a transposon-associated motif (TAM), or wherein the TnpB gene editing system treats one or more monogenic disorders or diseases.

36.-39. (canceled)

40. (canceled)

41. A method for editing a target sequence in the DNA of a host cell comprising delivering an effective amount of a pharmaceutical composition comprising at least one lipid nanoparticle (LNP) comprising at least one ionizable lipid selected from those listed in Tables (I), (II), (III), (IV) or (V); and at least one TnpB gene editing system, wherein the TnpB gene editing system comprises a nucleic acid sequence encoding a TnpB protein or functional variant thereof; and a TnpB ncRNA or a nucleic acid sequence encoding same, thereby installing an edit to the target sequence.

42.-47. (canceled)

48. The method for editing of claim 41, wherein the TnpB protein comprises a TnpB protein of Table A or functional fragment thereof, or an amino acid sequence having at least 85%, 90%, 95%, 99%, or up to 100% sequence identity with the TnpB protein of Table A or functional fragment thereof, or wherein the nucleic acid sequence encoding a TnpB protein or functional fragment thereof comprises nucleic acid sequence from Table B, or a nucleic acid sequence having at least 85%, 90%, 95%, 99%, or up to 100% sequence identity with a TnpB protein of Table B, or wherein the nucleic acid sequence encoding the TnpB protein comprises a linear or circular mRNA.

49.-50. (canceled)

51. The method for editing of claim 41, wherein the TnpB gene editing system further comprises a donor DNA template.

52. The method for editing of claim 51, wherein the donor DNA template is comprises single-stranded or double-stranded DNA, or wherein the donor DNA template comprises circular single-stranded DNA, or wherein the donor DNA template comprises an edit flanked by regions of homology to the regions upstream and downstream of a TnpB cut site.

53.-54. (canceled)

55. The method for editing of claim 41, wherein the edit comprises a double-strand cut, or comprises an insertion of 1 or more nucleobases, a deletion of 1 or more nucleobases, or a combination thereof, or comprises a transversion edit, or comprises a transition edit, or converts a T←→Cor A←→G, or converts a T→A or G, C→G or A, A→T or C, or G→C or T.

56.-60. (canceled)

61. The method for editing of claim 55, wherein the insertion or deletion comprises a whole exon or intron of a gene, or wherein the insertion or deletion comprises a whole or partial gene.

62. (canceled)

63. The method for editing of claim 41, wherein the TnpB gene editing system further comprises an accessory protein or a nucleotide sequence encoding the accessory protein.

64. The method for editing of claim 63, wherein the accessory protein comprises a nuclease, a deaminase, a recombinase, a reverse transcriptase, and an integrase, and/or wherein the accessory protein is fused to a TnpB protein to form a fusion protein, and/or wherein the TnpB protein comprises a fusion protein which comprises a TnpB protein and a deaminase, or comprises a TnpB protein and a reverse transcriptase, or comprises a TnpB protein and a recombinase, or comprises a TnpB protein and a nuclease, or comprises a TnpB protein and an integrase.

65.-70. (canceled)

71. The method for editing of claim 41 for ex vivo or in vivo delivery, or wherein the TnpB gene editing system recognizes a transposon-associated motif (TAM), or wherein the TnpB gene editing system treats one or more monogenic disorders or diseases.

72.-73. (canceled)
